(19) United States
(12) Patent Application Publication
(10) Pub. No.: US 2009/0271163 A1
(43) Pub. Date: Oct. 29, 2009

(54) Crystal structure of human factor VIII and uses thereof

(75) Inventors: Jacky Ngo, Brookline, MA (US); Mingdong Huang, Newton, MA (US); David A. Roth, Wellesley, MA (US); Barbara C. Furie, Wellesley, MA (US); Bruce Furie, Wellesley, MA (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 12/315,847

(22) Filed: Dec. 5, 2008

Related U.S. Application Data
(60) Provisional application No. 61/005,887, filed on Dec. 6, 2007.
(60) Provisional application No. 61/009,061, filed on Dec. 20, 2007.
(60) Provisional application No. 61/072,777, filed on Mar. 31, 2008.

(51) Int. Cl. C07K 14/755; G06G 7/58

(52) U.S. Cl. 703/11; 530/383

(57) ABSTRACT

The present invention provides crystals of human Factor VIII, in particular, a B-domain deleted human Factor VIII, and its three-dimensional structure. The present invention also provides the structural information of Factor VIII, and methods for identifying compounds that modulate Factor VIII activity, for determining structures of Factor VIII homologs or analogs, and for designing drug candidates for the treatment of hemophilia based on the structural information.

### RELATED APPLICATIONS

This application claims priority to U.S. Provisional Patent Application Ser. No. 61/005,887, filed on Dec. 6, 2007; U.S. Provisional Patent Application Ser. No. 61/009,061, filed on Dec. 20, 2007; and U.S. Provisional Patent Application Ser. No. 61/072,777, filed on Mar. 31, 2008, the entirety of each of which is hereby incorporated herein by reference.

### FIELD OF THE INVENTION

The present invention relates to human Factor VIII, in particular, B-domain deleted human Factor VIII, methods for its crystallization, crystals, 3-dimensional structures, and uses thereof.

### BACKGROUND OF THE INVENTION

Factor VIII is a protein cofactor that, when activated, forms a complex with Factor IXa on membrane surfaces to activate factor X during blood coagulation. This glycoprotein is encoded by a gene of 186 kb that is divided into 26 exons. Hemophilia A is caused by defects in the Factor VIII gene that leads to diminished or absent Factor VIII activity in blood, for example, missense mutations, nonsense mutations, gene deletions of varying size, inversions and splice junction mutations. The major treatment of the bleeding disorder associated with hemophilia involves the infusion of Factor VIII into the circulation of patients with hemophilia and the correction of hemostasis.

The three dimensional structure of Factor VIII is unknown.

### SUMMARY OF THE INVENTION

The present invention provides crystals of human Factor VIII, in particular, a B-domain deleted human Factor VIII, and its three-dimensional structure. The analysis of the three dimensional structure provides previously unknown structural information about the human Factor VIII protein which can be used for the design and development of novel, potent and specific therapeutics for the treatment of hemophilia and other thromboembolic disorders.

In one aspect, the present invention provides a composition comprising a human Factor VIII, in a crystalline form. In particular, the human Factor VIII lacks at least a portion of B-domain. In one embodiment, the crystalline human Factor VIII has a space group P4₁2₁2. In another embodiment, the crystalline human Factor VIII has unit cell dimensions a=b=134.11 Å, c=349.76 Å. In yet another embodiment, the crystalline human Factor VIII diffracts X-rays for a determination of structural coordinates to a resolution of about 4.0 Angstroms or below (e.g., about 3.8 Å or below, about 3.6 Å or below, about 3.4 Å or below, about 3.2 Å or below, about 3.0 Å or below, about 2.8 Å or below, about 2.5 Å or below, about 2.4 Å or below, about 2.3 Å or below, about 2.2 Å or below, about 2.1 Å or below, about 2.0 Å or below, about 1.9 Å or below, about 1.8 Å or below, about 1.7 Å or below, about 1.6 Å or below, about 1.5 Å or below, about 1.4 Å or below). In one particular example, the crystalline human Factor VIII diffracts X-rays for a determination of structural coordinates to a resolution at about 3.98 Angstroms.

In one embodiment, the human Factor VIII suitable for the present invention includes an amino acid sequence at least 70%, 75%, 80%, 85%, 90%, 95%, 98%, or 99% identical to SEQ ID NO:1. In a preferred embodiment, the human Factor VIII suitable for the present invention includes the amino acid sequence of SEQ ID NO:1. In another embodiment, the human Factor VIII suitable for the present invention includes an amino acid sequence at least 70%, 75%, 80%, 85%, 90%, 95%, 98%, or 99% identical to SEQ ID NO:2. In a preferred embodiment, the human Factor VIII suitable for the present invention includes the amino acid sequence of SEQ ID NO:2. More preferably, the human Factor VIII suitable for the present invention includes amino acid sequences of SEQ ID NO:1 and SEQ ID NO:2.

In another aspect, the present invention provides a computer-readable medium containing computer-readable data defined by structural coordinates of atoms of human Factor VIII according to Table 2, +/−a root mean square deviation for alpha carbon atoms of less than 2 Angstroms, or selected coordinates thereof. As used herein, a root mean square deviation for alpha carbon atoms of less than 2 Angstroms includes a root mean square deviation for alpha carbon atoms at about 2 Å or less, about 1.8 Å or less, about 1.5 Å or less, about 1.2 Å or less, about 1.0 Å or less, or about 0.5 Å or less.

In some embodiments, the computer-readable data is defined by structural coordinates of atoms of human Factor VIII according to Table 2, +/−a root mean square deviation for alpha carbon atoms of less than 2 Angstroms, or at least 20%, 25%, 30%, 35%, 40%, 45%, 50%, 55%, 60%, 65%, 70%, 75%, 80%, 85%, 90%, 95% of the coordinates thereof.

In some embodiments, the computer-readable data is defined by structural coordinates of one or more atoms selected from the group consisting of atoms of Lys 107, Glu 110, Asp 116, Glu 122, Asp 126, Asp 125, His 267, Cys 310, His 315, His 1954, Cys 2000, and His 2005. In other embodiments, the computer-readable data is defined by structural coordinates of one or more atoms selected from the group consisting of atoms of residues 558-565, 707-712 and 1811-1819 according to Table 2. In further embodiments, the computer-readable data are defined by three-dimensional structural coordinates of one or more Factor VIII domains selected from the group consisting of domain A1, A2, A3, C1 and C2.

In yet another aspect, the present invention provides a method for constructing a three-dimensional structural representation of a Factor VIIIa-Factor IXa complex, including a complex containing at least a region of Factor VIIIa and at least a region of Factor IXa. The method includes the steps of: (a) providing a three-dimensional structural representation of human Factor VIII, or a region thereof; (b) providing a three-dimensional structural representation of Factor IXa, or a region thereof, and (c) fitting the three-dimensional structural representation from step (a) to the three-dimensional structural representation from step (b).

In some embodiments, the three-dimensional structural representation of step (a) is defined by structural coordinates of atoms of human Factor VIII according to Table 2, +/−a root mean square deviation for alpha carbon atoms of less than 2 Angstroms, or selected coordinates thereof.

In some embodiments, the three-dimensional structural representation of step (a) is defined at least by structural coordinates of one or more atoms selected from the group consisting of atoms of residues 558-565, 707-712 and 1811-1819 according to Table 2.

In other embodiments, step (c) of the method of this aspect of the invention is based on one or more constraints selected from the group consisting of: (1) residues 558-565 of Factor VIIIa interact with residues 330-339 of Factor IXa; (2) residues 707-712 of Factor VIIIa interact with residues 301-303 of Factor IXa; (3) residues 1811-1819 of Factor VIIIa interact with the light chain of Factor IXa; (4) Phe 25 in the Gla domain of Factor IXa is juxtaposed with the light chain of Factor VIIIa; and (5) the Gla domain of Factor IXa is situated within a phospholipid membrane.

The present invention also provides computer-readable media containing computer-readable data defined by a three-dimensional structural representation of a complex comprising at least a region of Factor VIIIa and a region of Factor IXa constructed according to the methods as described in various embodiments above.

In still another aspect, the present invention provides a method for modifying human Factor VIIIa to alter its interaction with Factor IXa. The method includes the steps of: (a) providing a structural representation of human Factor VIIIa, or a region thereof; (b) fitting the structural representation of step (a) to a three-dimensional structural representation of Factor IXa, or a region thereof; and (d) computationally modifying the structural representation of step (a) to increase or decrease its interaction with the three-dimensional structural representation of Factor IXa, or a region thereof.

In some embodiments, the structural representation of step (a) is defined by structural coordinates of atoms of human Factor VIII according to Table 2, +/−a root mean square deviation for alpha carbon atoms of less than 2 Angstroms, or selected coordinates thereof.

In other embodiments, the structural representation of step (a) is defined at least by structural coordinates of one or more atoms selected from the group consisting of atoms of residues 558-565, 707-712 and 1811-1819 according to Table 2.

In some embodiments, the three-dimensional structural representation of Factor IXa, or a region thereof, is defined at least by the Gla domain of Factor IXa. In other embodiments, the three-dimensional structural representation of Factor IXa, or a region thereof, is defined at least by the light chain of Factor IXa.

In some embodiments, the structural representation of step (a) is modified to increase its interaction with the three-dimensional structural representation of Factor IXa, or a region thereof.

The present invention also provides modified human Factor VIII with increased interaction with Factor IXa according to the methods described in various embodiments above.

In one aspect, the present invention provides a method for evaluating the activity of a modified human Factor VIII. The method includes the steps of: (a) providing a structural representation of human Factor VIII, or a region thereof; (b) computationally modifying the structural representation of step (a) to introduce one or more amino acid modifications; and (c) evaluating the activity of the modified human Factor VIII based on the modified structural representation from step (b).

In some embodiments, the structural representation of step (a) is defined by coordinates of atoms of human Factor VIII according to Table 2, +/−a root mean square deviation for alpha carbon atoms of less than 2 Angstroms, or selected coordinates thereof.

In one embodiment, the evaluation of the activity of the modified human Factor VIII is based on an evaluation of its interaction with Factor IXa. In another embodiment, the evaluation of the activity of the modified human Factor VIII is based on an evaluation of its interaction with a phospholipid membrane. In yet another embodiment, the evaluation of the activity of the modified human Factor VIII is based on an evaluation of its interaction with von Willebrand Factor (vWF).

In some embodiments, the method of this aspect of the invention further includes a step of identifying a modified Factor VIII with an improved property. In one particular embodiment, the modified Factor VIII has increased plasma half-life.

In another aspect, the present invention provides a method of predicting a three dimensional structure of a human Factor VIII homologue or analogue of unknown structure. The method includes the steps of: (a) aligning an amino acid sequence of a target human Factor VIII homolog or analog of unknown structure with the amino acid sequence of human Factor VIII defined by coordinates according to Table 2, +/−a root mean square deviation for alpha carbon atoms of less than 2 Angstroms, or selected coordinates thereof, to match one or more homologous regions; (b) modeling the structure of the matched one or more homologous regions of the target human Factor VIII homolog or analog of unknown structure on the corresponding regions of the human Factor VIII as defined by coordinates according to Table 2, +/−a root mean square deviation for alpha carbon atoms of less than 2 Angstroms, or selected coordinates thereof; and (c) determining a structural conformation for said target human Factor VIII homolog or analog of unknown structure which substantially preserves the structure of said matched one or more homologous regions. In one embodiment, the method of this aspect of the invention further includes a step of evaluating the activity of the target human Factor VIII homolog or analog of unknown structure based on the structural conformation determined at step (c).

In still another aspect, the present invention provides a method for designing a mimetic compound of human Factor VIII. The method includes the steps of: (a) providing a selected human Factor VIII structure that is associated with a biological activity of human Factor VIII; (b) superimposing a three-dimensional structure of a compound on the selected human Factor VIII structure; and (c) modifying the three-dimensional structure of the compound such that the modified three-dimensional structure comprises a structural confirmation substantially mimicking the selected human Factor VIII structure. In one embodiment, the mimetic compound includes an antibody structure. In another embodiment, the method further includes the steps of: (d) synthesizing the modified compound from step (c); and (e) evaluating the activity of the modified compound.

The present invention also provides mimetic antibodies of human Factor VIII designed by the methods of this aspect of the invention.

In yet another aspect, the present invention provides a method for rational drug design. The method includes the steps of: (a) providing selected coordinates of a human Factor VIII structure; (b) providing a plurality of moieties; (c) fitting the structure of each of the plurality of moieties to the selected coordinates; (d) selecting one or more moieties that fit into the selected coordinates; and (e) assembling the one or more moieties selected from step (d) into a single molecule to form a candidate modulator molecule. In some embodiments, the selected coordinates of a human Factor VIII structure comprises one or more coordinates as defined in Table 2, +/−a root mean square deviation for alpha carbon atoms of less than 2 Angstroms.

In some embodiments, wherein the moieties suitable for the method of this aspect of the invention are selected from the group consisting of molecular fragments, small molecules, ligands designed de novo, and compounds known to bind Factor VIII or modified compounds thereof.

In other embodiments, the method of this aspect of the invention further includes the steps of: (f) obtaining or synthesizing the candidate modulator molecule; and (g) contacting the candidate modulator molecule with human Factor VIII to determine the ability of the candidate modulator molecule to interact with human Factor VIII.

In a further aspect, the present invention provides a method for producing a computer readable database including a structural representation of at least one compound capable of binding human Factor VIII. The method includes the steps of: (a) introducing into a computer program selected coordinates of a human Factor VIII structure; (b) fitting a three-dimensional model of at least one binding test compound on the selected coordinates; (d) assessing whether said test compound model fits spatially into the selected coordinates; and (e) storing a structural representation of a compound that fits into the selected coordinates.

The present invention also provides a computer readable database produced by the method of this aspect of the invention.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only, not for limitation.

FIG. 1A depicts a diagram of the domain organization of human Factor VIII and the B-domain deleted Factor VIII.

FIG. 1B depicts overall three dimensional structure of B-domain deleted Factor VIII.

FIG. 1C depicts overlaid structures of the C2 domain of B-domain deleted Factor VIII and the 1.5 Å resolution structure of the isolated C2 domain (dark grey) (Pratt, K. P., Shen, B. W., Takeshima, K., Davie, E. W., Fujikawa, K. & Stoddard, B. L. (1999) Nature 402, 439-442).

FIG. 1D depicts 2 Cu²⁺ and 1 Ca²⁺ binding sites identified in the B-domain deleted Factor VIII.

FIG. 2A-2D depict structural features of the A domains and C domains. FIG. 2A depicts the A1, A2 and A3 domains form a triangular heterotrimer around a pseudo-three-fold symmetry. FIG. 2B illustrates that both the C1 and C2 domains contain numerous basic or hydrophobic residues positioned in the hairpin loops at the base of the domains. FIG. 2C depicts the basic and hydrophobic nature of the putative lipid binding surface of the C domains. The solvent-accessible surface at the bottom of both C1 and C2 domains is shaded by electrostatic potential (8 kT/e) computed by APBS (Baker, N. A., Sept, D., Joseph, S., Holst, M. J. & McCammon, J. A. (2001) Proc Natl Acad Sci USA 98, 10037-41). FIG. 2D depicts the interaction between the C2 domain and the A1 (left inset) and the C1 (right inset) domains. The key residues involved in direct contact are indicated. The loop that connects the C1 and C2 domains is highlighted with dashed line in the right inset.

FIG. 3 depicts the comparison of Factor VIII and Factor Vai, and putative B-domain binding site. FIG. 3A depicts X-ray crystallographic structure of human B-domain deleted Factor VIII compared to Factor Vai (the activated protein C-inhibited Factor Va). FIG. 3B illustrates that the model of the heavy chain (dark grey) ends at residue Lys 713 and the light chain (light grey) starts at Phe 1691. Both termini are circled and the putative location of B-domain is outlined with an oval. This region covers the interaction sites between Factor VIIIa and Factor IXa (dash/dot line, diamond outline, rectangle outline).

FIGS. 4A and 4B depict the binding interface between the complex of Factor VIIIa and Factor IXa. FIG. 4A depicts the three different interaction sites with Factor IXa. FIG. 4B shows that the positions of residues contributed from different loops of C2 domain resemble that of the 330-339 α-helix of Factor IXa.

FIG. 5A depicts exemplary data reflecting the intermolecular energy, Eᵢₙₜₑᵣ (the sum of intermolecular van der Waals, electrostatic, and AIR energy terms) for 60 complex structures, after water refinement, as a function of their backbone rmsd from the lowest energy structure. FIGS. 5B and 5C depict a model of the Factor IXa-Factor VIIIa complex. FIG. 5B depicts front and side views of the complex of Factor VIIIa (dark and light grey) and Factor IXa (outlined in dashed line). Four putative membrane binding sites, including the bases of C1 and C2 domains, the A3 domain loop of Factor VIII and the Gla domain of Factor IXa, lie on the same plane and are darkly shaded (lower region of structures). The active site of Factor IXa is indicated (outlined in solid line). FIG. 5C depicts a diagram of the possible interaction of the Factor IXa-Factor VIIIa complex with phospholipid membrane surfaces.

FIG. 6 depicts an exemplary overlaid model of our B domain-deleted Factor VIII crystal structure (PDB ID: 3CDZ) and a crystal structure of a recombinant form of Factor VIII which consists of a heterodimer of peptides, respectively containing the A1-A2 and A3-C1-C2 domains, disclosed on Apr. 15, 2008 (PDB ID: 2R7E).

FIG. 7 illustrates exemplary electrostatic surface potential of Factor VIII. (A) Front; dark shaded region is positively charged. (B) Back; dark shaded region in the center is negatively charged.

### DETAILED DESCRIPTION OF THE INVENTION

The present invention provides crystals of human Factor VIII, in particular, a B-domain deleted human Factor VIII, and its three-dimensional structure. The present invention also provides the structural information of Factor VIII, and methods for identifying compounds that modulate Factor VIII activity, for determining structures of Factor VIII homologs or analogs, and for designing drug candidates for the treatment of hemophilia and other thromboembolic disorders based on the structural information.

Various aspects of the invention are described in detail in the following sections. The use of sections is not meant to limit the invention. Each section can apply to any aspect of the invention. In this application, the use of “or” means “and/or” unless stated otherwise.

### Factor VIII and Bleeding Disorder

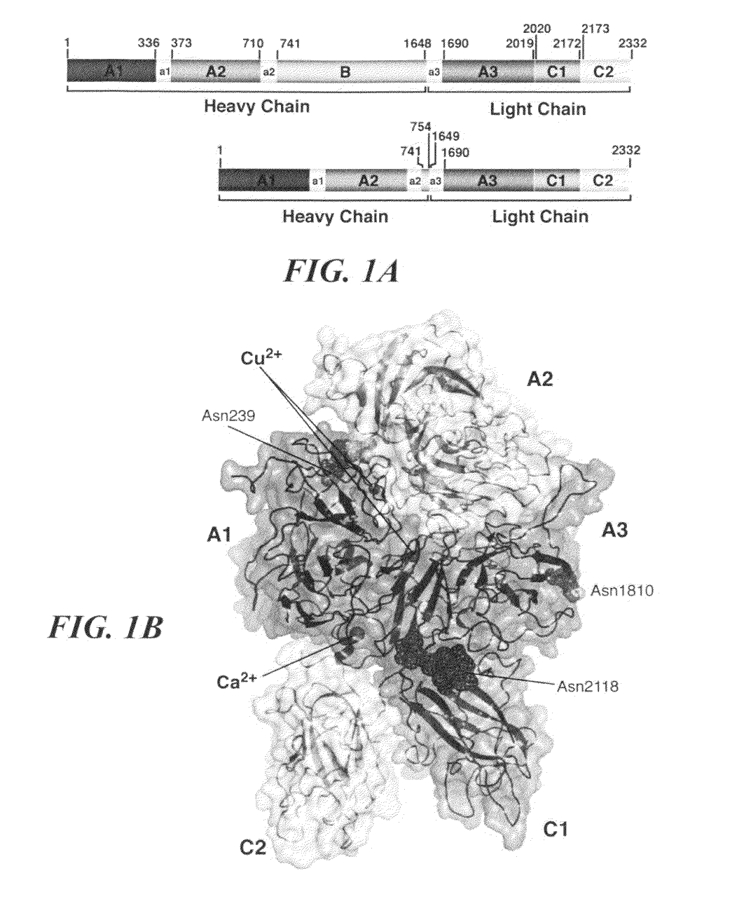

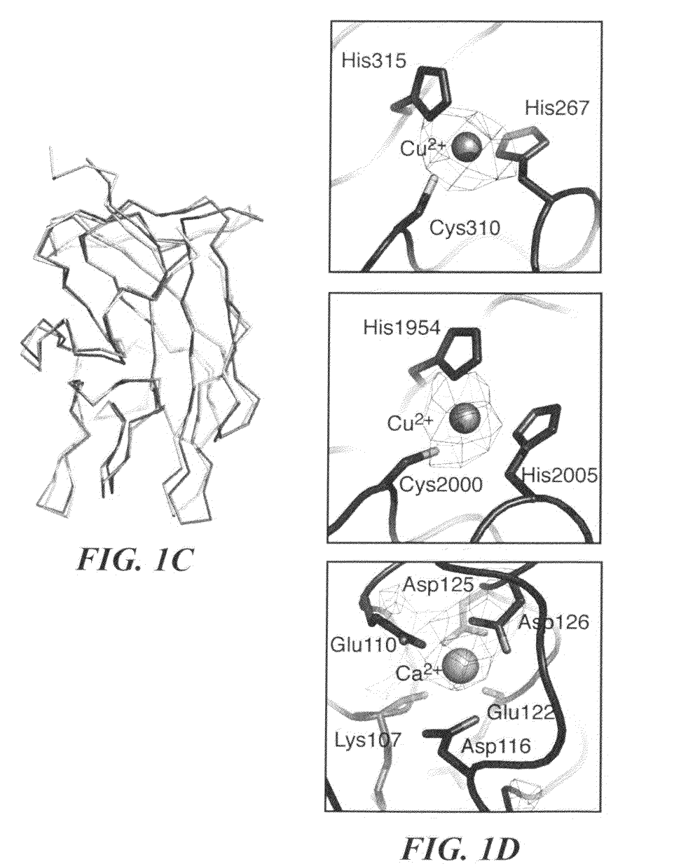

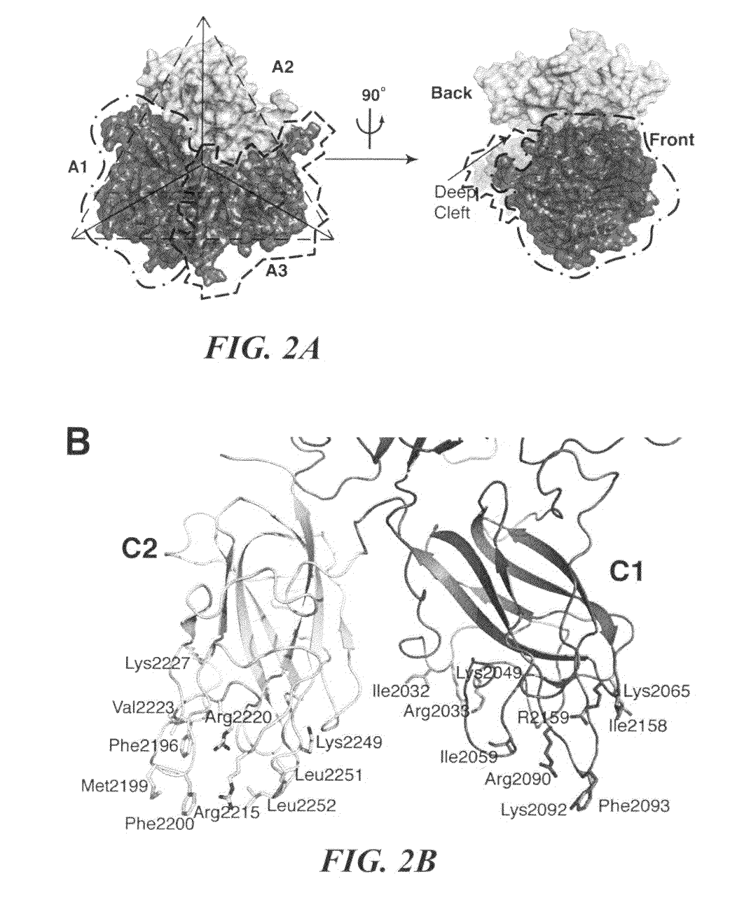

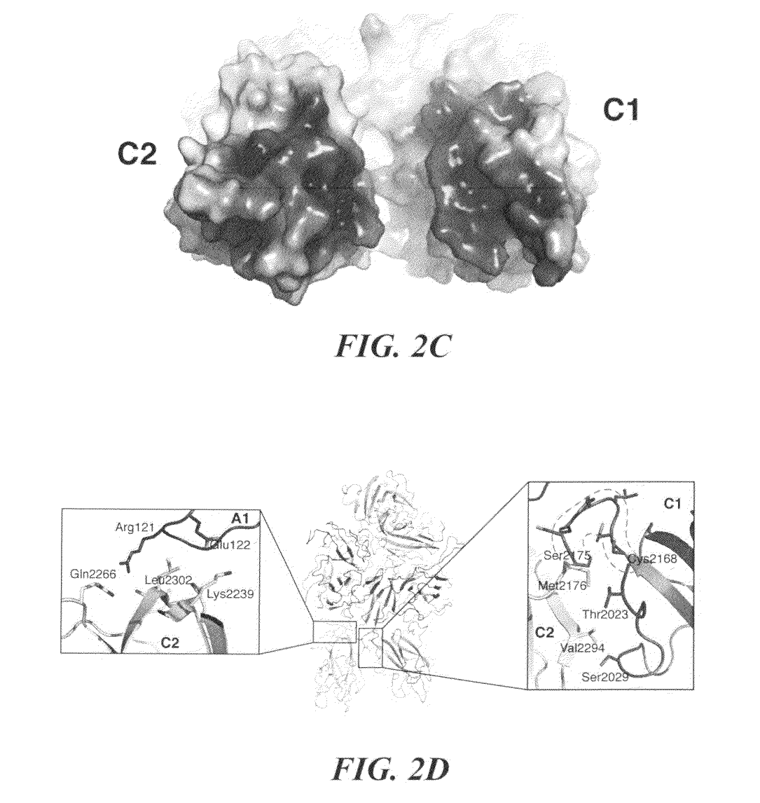

Factor VIII is a protein cofactor that, when activated, forms a complex with Factor IXa on membrane surfaces to activate factor X during blood coagulation (Furie, B. & Furie, B. C. (1988) Cell 53, 505-518). This glycoprotein is encoded by a gene of 186 kb that is divided into 26 exons (Gitschier, J., Wood, W. I., Goralka, T. M., Wion, K. L., Chen, E. Y., Eaton, D. H., Vehar, G. A., Capon, D. J. & Lawn, R. M. (1984) Nature 312, 326-30; Toole, J. J., Knopf, J. L., Wozney, J. M., Sultzman, L. A., Buecker, J. L., Pittman, D. D., Kaufman, R. J., Brown, E., Shoemaker, C., Orr, E. C. & et al. (1984) Nature 312, 342-7). Factor VIII is synthesized as a single polypeptide chain, including a 19-residue signal peptide. The mature Factor VIII contains 2,332 amino acid residues arranged within five domains organized as A1-A2-B-A3-C1-C2 (Gitschier, J., Wood, W. I., Goralka, T. M., Wion, K. L., Chen, E. Y., Eaton, D. H., Vehar, G. A., Capon, D. J. & Lawn, R. M. (1984) Nature 312, 326-30; Toole, J. J., Knopf, J. L., Wozney, J. M., Sultzman, L. A., Buecker, J. L., Pittman, D. D., Kaufman, R. J., Brown, E., Shoemaker, C., Orr, E. C. & et al. (1984) Nature 312, 342-7) (FIG. 1A). A diagram of the domain organization of human Factor VIII and the B-domain deleted Factor VIII is shown in FIG. 1A. According to Lenting et al. (1998) Blood 92, 3983-3996, domain A1 corresponds to amino acids 1-336. Domain A2 corresponds to amino acids 373-710. Domain B corresponds to amino acids 741-1648. Domain A3 corresponds to amino acids 1690-2019. Domain C1 corresponds to amino acids 2020-2172. Domain C2 corresponds to amino acids 2173-2332. In addition, the A domains are bordered by acidic regions a1 corresponding to amino acids 337-372, a2 corresponds to amino acids 711-740, or a3 corresponds to amino acids 1649-1689.

Factor VIII circulates in the blood as a heterodimer composed of two polypeptide chains, a light chain with a molecular weight of about 80,000 and a heterogeneous heavy chain with a molecular weight varying between about 90,000 and 200,000, both derived from the single peptide chain. A region of the C2 domain defines the membrane-binding properties of Factor VIII and the site of interaction with von Willebrand factor (Pratt, K. P., Shen, B. W., Takeshima, K., Davie, E. W., Fujikawa, K. & Stoddard, B. L. (1999) Nature 402, 439-442). Factor VIII is inactive or minimally active as a cofactor in blood coagulation, but is converted into its active cofactor form by proteolytic cleavage. Although active Factor VIII can be formed from cleavage at Arg 372 and Arg 1689, it is generally appreciated that activated Factor VIII is generated from 3 cleavage events (Arg 372, Arg 1689, and Arg 740).

Activated Factor VIII (Factor VIIIa) acts as a cofactor for activated Factor IX (Factor IXa) to accelerate the conversion of factor X to activated factor X (factor Xa). Factor Xa converts prothrombin into thrombin. Thrombin then converts fibrinogen into fibrin and a clot is formed.

Classic hemophilia (also known as hemophilia A) is caused by a defect in the Factor VIII gene that leads to diminished or absent Factor VIII activity in blood. A heterogeneous genetic disease, hemophilia A has been associated with missense mutations, nonsense mutations, gene deletions of varying size, inversions and splice junction mutations (Furie, B. & Furie, B. C. (1990) Semin Hematol 27, 270-85; Graw, J., Brackmann, H. H., Oldenburg, J., Schneppenheim, R., Spannagl, M. & Schwaab, R. (2005) Nat Rev Genet. 6, 488-501). The major treatment of the bleeding disorder associated with hemophilia involves the infusion of Factor VIII into the circulation of patients with hemophilia and the correction of hemostasis (Mannucci, P. M. & Tuddenham, E. G. (2001) N Engl J Med 344, 1773-9; Key, N. S. & Negrier, C. (2007) Lancet 370, 439-48).

### B-Domain Deleted Human Factor VIII

Based upon the observation that the B-domain of porcine Factor VIII shows no sequence homology to the B-domain of human Factor VIII yet porcine and human Factor VIII have similar specific coagulant activities when evaluated in human plasma, engineered B-domain deleted human Factor VIII was shown to have full biological activity and could be expressed in heterologous cells with improved expression efficiency relative to that of the full-length molecule (Toole, J. J., Pittman, D. D., Orr, E. C., Murtha, P., Wasley, L. C. & Kaufman, R. J. (1986) Proc Natl Acad Sci USA 83, 5939-42). The structural heterogeneity of B-domain deleted Factor VIII is significantly less than that for full length Factor VIII, with a heavy chain of 90,000 and a light chain of 80,000 molecular weight (FIG. 1A).

As used herein, a “B-domain deleted human Factor VIII” includes a Factor VIII, or a structural or functional variant, that lacks at least a portion or the entirety of the B-domain. As used herein, the B-domain of Factor VIII corresponds to amino residues 741 thru and including 1648 of human Factor VIII.

Thus, a B-domain deleted human Factor VIII suitable for the invention includes a heavy chain and a light chain. The heavy chain of human Factor VIII includes amino acid residues 1-740 (A1-a1-A2-a2) and the light chain of human Factor VIII includes amino acid residues 1649-2332 (a3-A3-C1-C2). This sequence of the heavy chain of human Factor VIII is shown below as SEQ ID NO:1 and the sequence of the light chain of human Factor VIII is shown below as SEQ ID NO:2.

The heavy chain of human Factor VIII (including residues 1-740) (SEQ ID NO:1)

ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETKTREAIQHESGILGP
LLYGEVGDTLLIIFKNQASRPYNIYPHGITDVRPLYSRRLPKGVKHLKDF
PILPGEIFKYKWTVTVEDGPTKSDPRCLTRYYSSFVNMERDLASGLIGPL
LICYKESVDQRGNQIMSDKRNVILFSVFDENRSWYLTENIQRFLPNPAGV
QLEDPEFQASNIMHSINGYVFDSLQLSVCLHEVAYWYILSIGAQTDFLSV
FFSGYTFKHKMVYEDTLTLFPFSGETVFMSMENPGLWILGCHNSDFRNRG
MTALLKVSSCDKNTGDYYEDSYEDISAYLLSKNNAIEPR

The light chain of human Factor VIII (including residues 1649-2332) (SEQ ID NO:2):

EITRTTLQSDQEEIDYDDTISVEMKKEDFDIYDEDENQSPRSFQKKTRHY
FIAAVERLWDYGMSSSPHVLRNRAQSGSVPQFKKVVFQEFTDGSFTQPLY
RGELNEHLGLLGPYIRAEVEDNIMVTFRNQASRPYSFYSSLISYEEDQRQ
GAEPRKNFVKPNETKTYFWKVQHHMAPTKDEFDCKAWAYFSDVDLEKDVH
SGLIGPLLVCHTNTLNPAHGRQVTVQEFALFLTIFDETKSWYFTENMERN
CRAPCNIQMEDPTFKENYRFHAINGYIMDTLPGLVMAQDQRIRWYLLSMG
SNENIHSIHFSGHVFTVRKKEEYKMALYNLYPGVFETVEMLPSKAGIWRV
ECLIGEHLHAGMSTLFLVYSNKCQTPLGMASGHIRDFQITASGQYGQWAP
KLARLHYSGSINAWSTKEPFSWIKVDLLAPMIIHGIKTQGARQKFSSLYI
SQFIIMYSLDGKKWQTYRGNSTGTLMVFFGNVDSSGIKHNIFNPPIIARY
IRLHPTFHYSIRSTLRMELMGCDLNSCSMPLGMESKAISDAQITASSYFT
NMFATWSPSKARLHLQGRSNAWRPQVNNPKEWLQVDFQKTMKVTGVTTQG
VKSLLTSMYVKEFLISSSQDGHQWTLFFQNGKVKVFQGNQDSFTPVVNSL
DPPLLTRYLRIHPQSWVHQIALRMEVLGCEAQDLY

A B-domain deleted Factor VIII suitable for the invention may also contain modified or mutated heavy and/or light chains. Modified or mutant heavy or light chains may be generated by replacing at least one amino acid residue in a native polypeptide with a different amino acid residue, or by adding or deleting amino acid residues within the native polypeptide or at the N- or C-terminus of the native polypeptide. Preferably, the B-domain deleted Factor VIII including modified heavy and/or light chains has substantially the same three-dimensional structure. By having substantially the same three-dimensional structure is meant having a set of atomic structure coordinates that have a root-mean-square deviation of less than or equal to about 2 Å when superimposed with the atomic structure coordinates of the native protein, or a region thereof, from which the mutant is derived when at least about 50% to 100% of the alpha carbon atoms of the corresponding native protein, or a region thereof, are included in the superposition.

Amino acid substitutions, deletions and additions which do not significantly interfere with the three-dimensional structure of Factor VIII will depend, in part, on the region of Factor VIII where the substitution, addition or deletion occurs. In highly variable regions of the molecule, non-conservative substitutions as well as conservative substitutions may be tolerated without significantly disrupting the three-dimensional, structure of the molecule. In highly conserved regions, or regions containing significant secondary structure, conservative amino acid substitutions are preferred.

Conservative amino acid substitutions are well known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

For proteins obtained in whole or in part by chemical synthesis, the selection of amino acids available for substitution or addition is not limited to the genetically encoded amino acids. Indeed, the mutants described herein may contain non-genetically encoded amino acids. Conservative amino acid substitutions for many of the commonly known non-genetically encoded amino acids are well known in the art. Conservative substitutions for other amino acids can be determined based on their physical properties as compared to the properties of the genetically encoded amino acids.

In some instances, it may be particularly advantageous or convenient to substitute, delete and/or add amino acid residues to a native protein in order to provide convenient cloning sites in cDNA encoding the polypeptide, to aid in purification of the polypeptide, and for crystallization of the polypeptide. Such substitutions, deletions and/or additions which do not substantially alter the three dimensional structure of Factor VIII will be apparent to those of ordinary skill in the art.

Thus, a human Factor VIII suitable for the present invention includes an amino acid sequence at least 70%, 75%, 80%, 85%, 90%, 95%, 98%, or 99% identical to SEQ ID NO:1. In one particular embodiment, the human Factor VIII suitable for the present invention includes the amino acid sequence of SEQ ID NO:1. In another embodiment, the human Factor VIII suitable for the present invention includes an amino acid sequence at least 70%, 75%, 80%, 85%, 90%, 95%, 98%, or 99% identical to SEQ ID NO:2. In one particular embodiment, the human Factor VIII suitable for the present invention includes the amino acid sequence of SEQ ID NO:2. More particularly, the human Factor VIII suitable for the present invention includes amino acid sequences of SEQ ID NO:1 and SEQ ID NO:2.

In addition, the human Factor VIII suitable for the invention may also contain a linker, for example, at the C-terminus of the heavy chain, that connects the heavy chain and the light chain. A suitable linker may be derived from the amino acid sequence of the B-domain. For example, a linker may contain amino acids derived from the N-terminal region and/or the C-terminal region of the B-domain. One such exemplary linker sequence is as follows: SFSQNPPVLKRHQR (SEQ ID NO:3). Alternatively, a linker may incorporate artificial amino acid sequences other than any naturally-occurring sequences and is generally designed to be flexible or to interpose a structure, such as an alpha-helix, between the two protein moieties.

### Three-Dimensional Structure Determination Using X-Ray Crystallography

X-ray crystallography is a method of solving the three dimensional structures of molecules. The structure of a molecule is calculated from X-ray diffraction patterns using a crystal as a diffraction grating. Three dimensional structures of protein molecules arise from crystals grown from a concentrated aqueous solution of that protein. The process of X-ray crystallography can include the following steps: (a) synthesizing and isolating (or otherwise obtaining) a protein; (b) growing a crystal from an aqueous solution comprising the protein with or without a modulator; and (c) collecting X-ray diffraction patterns from the crystals, determining unit cell dimensions and symmetry, determining electron density, fitting the amino acid sequence of the protein to the electron density, and refining the structure.

Preparation of B-Domain Deleted Human Factor VIII

A B-domain deleted human Factor VIII described herein may be chemically synthesized in whole or part using techniques that are well-known in the art (see, e.g., Creighton (1983) Biopolymers 22(1):49-58). Alternatively, methods which are well known to those skilled in the art can be used to construct expression vectors containing the Factor VIII coding sequence and appropriate transcriptional/translational control signals. These methods include in vitro recombinant DNA techniques, synthetic techniques and in vivo recombination/genetic recombination. See, for example, the techniques described in Maniatis, T (1989). Molecular cloning: A laboratory Manual. Cold Spring Harbor Laboratory, New York. Cold Spring Harbor Laboratory Press; and Ausubel, F. M. et al. (1994) Current Protocols in Molecular Biology. John Wiley & Sons, Secaucus, N.J.

B-domain deleted human Factor VIII can be expressed in a variety of mammalian cell lines including, but not limited to, human embryonic kidney (HEK) 293, Chinese hamster ovary (CHO), monkey kidney (COS), HTH080, C10, HeLa, baby hamster kidney (BHK), 3T3, C127, CV-1, HaK, NS/O, and L-929 cells. B-domain deleted human Factor VIII can also be expressed in a variety of non-mammalian host cells such as, for example, insect (e.g., Sf-9, Sf-21, Hi5), plant (e.g., Leguminosa, cereal, or tobacco), yeast (e.g., S. cerivisae, P. pastoris), prokaryote (e.g., E. Coli, B. subtilis and other Bacillus spp., Pseudomonas spp., Streptomyces spp), or fungus. Detailed preparation of B-domain deleted human Factor VIII was described in Sandberg et al. (2001) Seminars in Hematology, Vol. 38, No. 2, Suppl. 4: pp 4-12; and Eriksson et al. (2001) Semin. Hematol., 38:24-31, the teachings of both of which are hereby incorporated by reference.

Crystal Growth

Typically, crystals are grown from an aqueous solution containing the purified and concentrated protein by a variety of techniques. Suitable techniques include batch, liquid, bridge, dialysis, vapor diffusion, and hanging drop methods. McPherson (1982) John Wiley, New York; McPherson (1990) Eur. J. Biochem. 189:1-23; Webber (1991) Adv. Protein Chem. 41:1-36, incorporated by reference herein in their entireties, including all figures, tables, and drawings.

The crystals of the invention are, in general, grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases. For crystals of the invention, exemplary crystallization conditions are described in the Examples. Those of ordinary skill in the art will recognize that the exemplary crystallization conditions can be varied. Such variations may be used alone or in combination. In addition, other crystallizations may be found, e.g., by using crystallization screening plates to identify such other conditions.

Derivative crystals of the invention can be obtained by soaking original crystals in mother liquor containing salts of heavy metal atoms. Heavy metal atoms useful for providing derivative crystals include, by way of example and not limitation, gold, mercury, selenium, etc. It has been found that soaking an original crystal in a solution containing about 0.1 mM to about 5 mM thimerosal, 4-chloromeruribenzoic acid or KAu(CN)₂ for about 2 hr to about 72 hr provides derivative crystals suitable for use as isomorphous replacements in determining the X-ray crystal structure of Factor VIII.

Co-crystals of the invention can be obtained by soaking a crystal of Factor VIII in mother liquor containing compound that binds Factor VIII, or can be obtained by co-crystallizing the Factor VIII protein in the presence of a binding compounds. Thus, the co-crystals generally comprise a crystalline Factor VIII, or a region thereof, in association with one or more compounds. The association may be covalent or non-covalent. Such compounds include, but are not limited to, cofactors, substrates, substrate analogues, inhibitors, allosteric effectors, etc.

Generally, co-crystallization of Factor VIII and binding compound can be accomplished using conditions identified for crystallizing the corresponding Factor VIII without binding compound. It is advantageous if a plurality of different crystallization conditions have been identified for Factor VIII, and these can be tested to determine which condition gives the best co-crystals. It may also be beneficial to optimize the conditions for co-crystallization.

Determining Unit Cell Dimensions and the Three Dimensional Structure

Once the crystal is grown, it can be placed in a glass capillary tube or other mounting device and mounted onto a holding device connected to an X-ray generator and an X-ray detection device. Collection of X-ray diffraction patterns are well documented by those in the art. See, e.g., Ducruix and Geige, (1992), IRL Press, Oxford, England, and references cited therein. A beam of X-rays enters the crystal and then diffracts from the crystal. An X-ray detection device can be utilized to record the diffraction patterns emanating from the crystal. Although the X-ray detection device on older models of these instruments is a piece of film, modern instruments digitally record X-ray diffraction scattering. X-ray sources can be of various types, but advantageously, a high intensity source is used, e.g., a synchrotron beam source.

Methods for obtaining the three dimensional structure of the crystalline form of a peptide molecule or molecule complex are well known in the art. See, e.g., Ducruix and Geige, (1992), IRL Press, Oxford, England, and references cited therein. The following are steps in the process of determining the three dimensional structure of a molecule or complex from X-ray diffraction data.

After the X-ray diffraction patterns are collected from the crystal, the unit cell dimensions and orientation in the crystal can be determined. They can be determined from the spacing between the diffraction emissions as well as the patterns made from these emissions. The unit cell dimensions are characterized in three dimensions in units of Angstroms (one Å=10⁻¹⁰ meters) and by angles at each vertices. The symmetry of the unit cell in the crystals is also characterized at this stage. The symmetry of the unit cell in the crystal simplifies the complexity of the collected data by identifying repeating patterns. Application of the symmetry and dimensions of the unit cell is described below.

Each diffraction pattern emission is characterized as a vector and the data collected at this stage of the method determine the amplitude of each vector. The phases of the vectors can be determined using multiple techniques. In one method, heavy atoms can be soaked into a crystal, a method called isomorphous replacement, and the phases of the vectors can be determined by using these heavy atoms as reference points in the X-ray analysis. (Otwinowski, (1991), Daresbury, United Kingdom, 80-86). The isomorphous replacement method usually utilizes more than one heavy atom derivative. In another method, the amplitudes and phases of vectors from a crystalline polypeptide with an already determined structure can be applied to the amplitudes of the vectors from a crystalline polypeptide of unknown structure and consequently determine the phases of these vectors. This second method is known as molecular replacement and the protein structure which is used as a reference must have a closely related structure to the protein of interest. (Naraza (1994) Proteins 11:281-296). For example, the structural information from any isolated Factor VIII domain, or related proteins such as, Factor Va or ceroluplasmin, can be used as references for the molecular replacement analysis.

Once the phases of the vectors describing the unit cell of a crystal are determined, the vector amplitudes and phases, unit cell dimensions, and unit cell symmetry can be used as terms in a Fourier transform function. The Fourier transform function calculates the electron density in the unit cell from these measurements. The electron density that describes one of the molecules or one of the molecule complexes in the unit cell can be referred to as an electron density map. The amino acid structures of the sequence or the molecular structures of compounds complexed with the crystalline polypeptide may then be fitted to the electron density using a variety of computer programs. This step of the process is sometimes referred to as model building and can be accomplished by using computer programs such as Turbo/FRODO or “O” (Jones (1985) Methods in Enzymology 115:157-171), or AMoRe (Navaza, (1994) Acta Cryst. A50:157-163)

A theoretical electron density map can then be calculated from the amino acid structures fit to the experimentally determined electron density. The theoretical and experimental electron density maps can be compared to one another and the agreement between these two maps can be described by a parameter called an R-factor. A low value for an R-factor describes a high degree of overlapping electron density between a theoretical and experimental electron density map.

The R-factor is then minimized by using computer programs that refine the theoretical electron density map. A computer program such as X-PLOR can be used for model refinement by those skilled in the art. Briinger (1992) Nature 355:472-475. Other suitable computer programs such as or REFMAC are well known in the art. Refinement may be achieved in an iterative process. A first step can entail altering the conformation of atoms defined in an electron density map. The conformations of the atoms can be altered by simulating a rise in temperature, which will increase the vibrational frequency of the bonds and modify positions of atoms in the structure. At a particular point in the atomic perturbation process, a force field, which typically defines interactions between atoms in terms of allowed bond angles and bond lengths, Van der Waals interactions, hydrogen bonds, ionic interactions, and hydrophobic interactions, can be applied to the system of atoms. Favorable interactions may be described in terms of free energy and the atoms can be moved over many iterations until a free energy minimum is achieved. The refinement process can be iterated until the R-factor reaches a minimum value.

The three dimensional structure of the molecule or molecule complex is described by atoms that fit the theoretical electron density characterized by a minimum R-value. A file can then be created for the three dimensional structure that defines each atom by coordinates in three dimensions. An example of such a structural coordinate file is shown in Table 2.

### Structures of B-Domain Deleted Human Factor VIII

The present invention provides three-dimensional structures and atomic structure coordinates of B-domain deleted human Factor VIII as determined by X-ray crystallography. The specific methods used to obtain the structure coordinates are provided in the examples. Exemplary atomic structure coordinates of B-domain deleted human Factor VIII are listed in Table 2.

Those having skill in the art will recognize that atomic structure coordinates as determined by X-ray crystallography are not without error. Thus, it is to be understood that any set of structure coordinates obtained for crystals of B-domain deleted human Factor VIII, or a region thereof, that have a root mean square deviation (“rmsd”) of less than or equal to about 2 Å when superimposed, using backbone atoms (e.g. N, Ca, C or O), on the corresponding structure coordinates listed in Table 2 are considered to be identical with the structure coordinates listed in the Table 2 when at least about 50% to 100% of the corresponding backbone atoms are included in the superposition. As used herein, a root mean square deviation for alpha carbon atoms of less than 2 Angstroms includes a root mean square deviation for alpha carbon atoms at about 2 Å or less, at about 1.8 Å or less, at about 1.5 Å or less, at about 1.2 Å or less, at about 1.0 Å or less, or at about 0.5 Å or less.

The overall three dimensional structure of B-domain deleted Factor VIII is illustrated in FIG. 1B. The structure includes five globular domains with overall dimensions of about 120 Å by 75 Å. The A1 domain (residues 1-336) and the a1 acidic region (residues 337-372) are depicted. The A2 domain (residues 373-710) and the a2 acidic region (residues 711-740) are depicted. These regions (A1, a1, A2, and a2) are part of the heavy chain. The A3 domain (residues 1690-2019), and the C1 domain (residues 2020-2172) and the C2 domain (residues 2173-2332) are part of the light chain are depicted here. The a3 acidic region is disordered and not included in this structure. The structure contains 2 Cu²⁺ ions, 1 Ca²⁺ ion and three carbohydrate moieties.

FIG. 1C depicts overlaid structures of the C2 domain of B-domain deleted Factor VIII provided by the present invention and the 1.5 Å resolution structure of the isolated C2 domain (grey) (Pratt, K. P., Shen, B. W., Takeshima, K., Davie, E. W., Fujikawa, K. & Stoddard, B. L. (1999) Nature 402, 439-442).

FIG. 1D depicts 2 Cu²⁺ and 1 Ca²⁺ binding sites identified in the B-domain deleted Factor VIII. Anomalous electron density map of the metal ions are contoured at 46. The upper panel in FIG. 1D illustrates the Cu²⁺ binding site in A1 domain. The middle panel shows the Cu²⁺ binding site in A3 domain. Each Cu²⁺ is liganded by two histidine and one cysteine residue with a trigonal planar coordination geometry. The lower panel in FIG. 1D illustrates that Ca²⁺ ion is liganded by carboxyl groups of aspartate and glutamate residues as well as two backbone carbonyl oxygens.

The A1, A2 and A3 domains each consists of two β-barrel structures that resemble the fold of a typical cupredoxin-type domain. All three A domains share high structural homology with each other and the A domains of ceruloplasmin. The A domains form a triangular heterotrimer where A1 and A3 domains serve as the base and interact with the C2 and C1 domains respectively. The C1 and C2 domains are defined by a distorted β-barrel and are structurally homologous with each other. At the base of the Factor VIII structure, both C domains reveal membrane binding features.

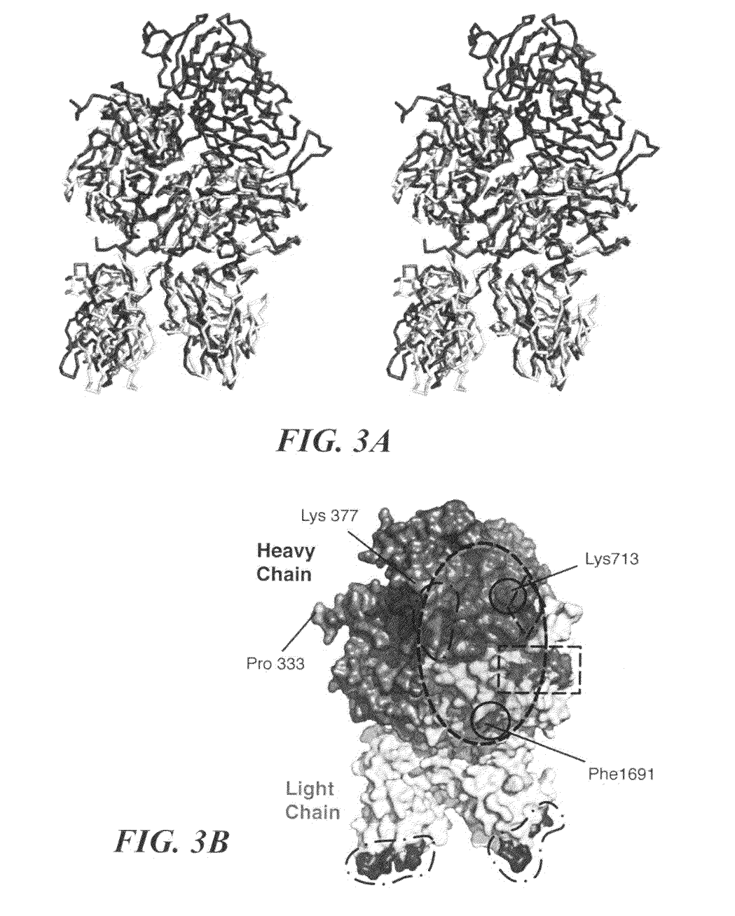

FIGS. 2A-2D depict structural features of the A domains and C domains. As illustrated in FIG. 2A, the A1, A2 and A3 domains form a triangular heterotrimer around a pseudo-three-fold symmetry axis. Side view of the A domain heterotrimer shows that the front surface is relatively flat when compared to the back face, which contains a deep cleft formed by three large loops. As shown in FIG. 2B, both of the C1 and C2 domains contain numerous basic or hydrophobic residues positioned in the hairpin loops at the base of the domains. As an indication of the basic and hydrophobic nature of the putative lipid binding surface of the C domains, the solvent-accessible surface at the bottom of both C1 and C2 domains is shaded by electrostatic potential 8 kT/e) computed by APBS (Baker, N. A., Sept, D., Joseph, S., Holst, M. J. & McCammon, J. A. (2001) Proc Natl Acad Sci USA 98, 10037-41) (FIG. 2C). FIG. 2D shows that the C2 domain has few interactions with the A1 (left inset) and the C1 (right inset) domains. The key residues involved in direct contact are indicated. The loop that connects the C1 and C2 domains is highlighted with dashed line in the right inset.

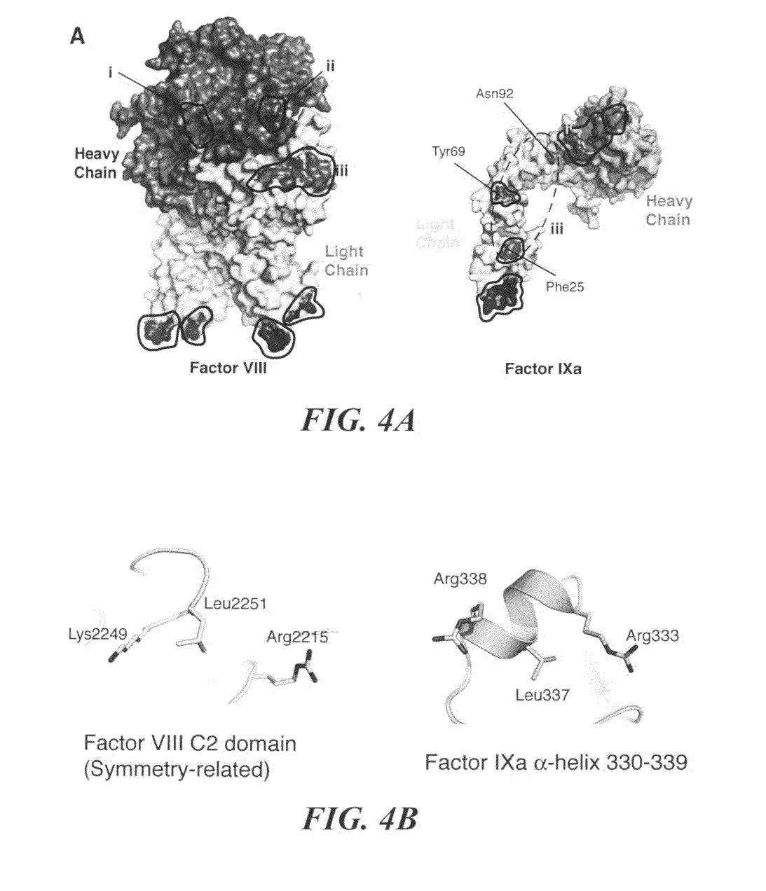

The domain organization in Factor VIII is homologous to the activated protein C-inactivated Factor Va (Factor Vai) structure (Adams et al., Proc. Natl. Acad. Sci. USA, 101:8918, 2004) except that it also includes the A2 domain. FIGS. 3A and 3B depict the comparison of Factor VIII and Factor Vai and putative B-domain binding site. FIG. 3A depicts X-ray crystallographic structure of human B-domain deleted Factor VIII compared to Factor Vai. FIG. 3A shows the superimposition of the carbon backbones of B-domain deleted Factor VIII (dark grey shaded) and Factor Vai (lightly shaded). Factor Vai shares the same domain organization as B-domain deleted Factor VIII but does not include the A2 domain. As illustrated in FIG. 1B, the model of the heavy chain (dark grey) ends at residue Lys 713 and the light chain (light grey) starts at Phe 1691. Both termini are circled and the putative location of B-domain is outlined in an oval. This region covers the interaction sites between Factor VIIIa and Factor IXa (dash/dot line, diamond outline, rectangle outline).

### Structural Model of the Complex of Factor VIIIa and Factor IXa

The absence of B-domain and the disorder of the loops containing the cleavage sites necessary for Factor VIII activation suggest that the our current structural model resembles the covalent structure of Factor VIIIa. Thus, the structural model of the present invention can be used to construct a three-dimensional structural model of a Factor VIIIa-Factor IXa complex, including a complex containing at least a region of Factor VIIIa and at least a region of Factor IXa. In general, the method includes the steps of: (a) providing a three-dimensional structural representation of human Factor VIII, or a region thereof; (b) providing a three-dimensional structural representation of Factor IXa, or a region thereof; and (c) fitting the three-dimensional structural representation from step (a) to the three-dimensional structural representation from step (b).

The assembly of the Factor IXa-Factor VIIIa complex normally involves the binding of Factor VIIIa and Factor IXa on phospholipid membrane surfaces in the presence of calcium ions. Based upon homology modeling (Autin, L., Miteva, M. A., Lee, W. H., Mertens, K., Radtke, K. P. & Villoutreix, B. O. (2005) J Thromb Haemost 3, 2044-56), the analysis of naturally occurring hemophilia A and B mutations or mutations introduced by site-specific mutagenesis (Mannucci, P. M. & Tuddenham, E. G. (2001) N Engl J Med 344, 1773-9; Jenkins, P. V., Dill, J. L., Zhou, Q. & Fay, P. J. (2004) Biochemistry 43, 5094-101; Nishimura, H., Takeya, H., Miyata, T., Suchiro, K., Okamura, T., Niho, Y. & Iwanaga, S. (1993) J Biol Chem 268, 24041-6; Hughes, P. E., Morgan, G., Rooney, E. K., Brownlee, G. G. & Handford, P. (1993) J Biol Chem 268, 17727-33), cross-linking studies (Blostein, M. D., Furie, B. C., Rajotte, I. & Furie, B. (2003) J Biol Chem 278, 31297-302) and inhibition with synthetic peptides (Lenting, P. J., van de Loo, J. W., Donath, M. J., van Mourik, J. A. & Mertens, K. (1996) J Biol Chem 271, 1935-40), the binding surface on Factor VIIIa is thought to involve the A2 and A3 domains interacting with multiple domains on Factor IXa. Thus, a model of the Factor IXa-Factor VIIIa complex using our Factor VIII structure and the x-ray crystal structure of porcine Factor IXa backbone may be constructed using one or more of the following constraints: (1) residues 558-565 of Factor VIII interact with the 330-339 helix of Factor IXa; (2) 707-712 of Factor VIIIa binds to Factor IXa residues 301-303; (3) residues 1811-1819 of Factor VIII interact with the light chain of Factor IXa; (4) Phe 25 in the Gla domain of Factor IX is juxtaposed with the light chain of Factor VIII; and (5) the Gla domain of Factor IXa is situated within the phospholipid membrane, forming non-covalent interactions between the phosphoserine head group and fatty acid chains of the phospholipid bilayer and the hydrophobic patch and the Gla residues within the Gla domain of Factor IXa.

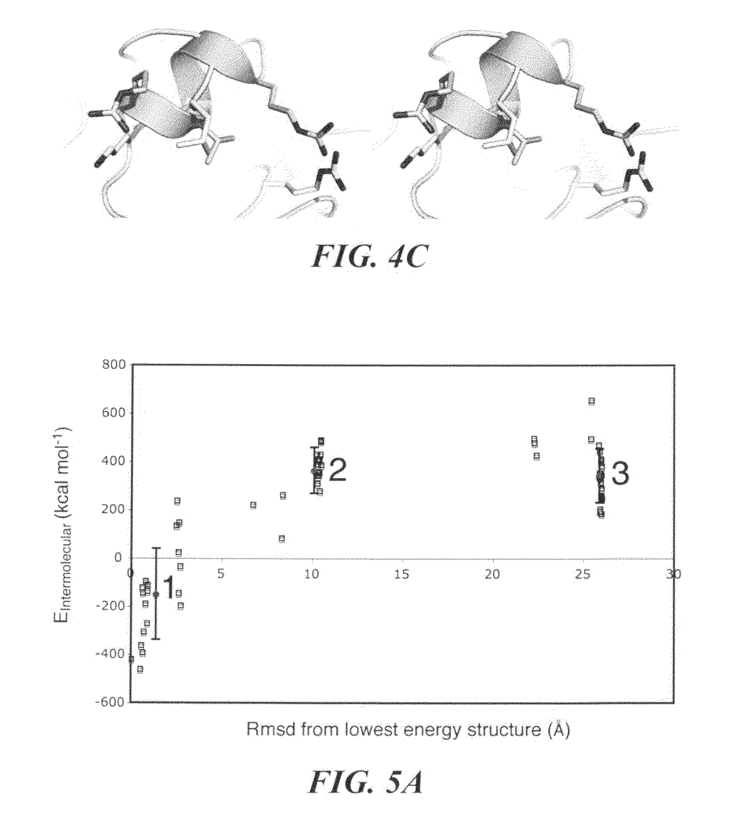

FIGS. 4A and 4B illustrate the binding interface between Factor VIII and Factor IXa. In FIG. 4A, three different interaction sites with Factor IXa were identified on Factor VIIIa in previous studies and are indicated by i, ii, and iii respectively. The complementary binding sites on Factor IXa are labeled accordingly. Region i includes residues 558-565 on Factor VIII and the 330-339 helix on Factor IXa. Region ii includes residue around 712 on Factor VIII and 301-303 on Factor IXa. Region iii on Factor VIII represents the binding site (1811-1818) that is suggested to be responsible for the high affinity interaction between Factor VIIIa and Factor IXa. Residues on the light chain of Factor IXa that have been shown to be important for binding include Phe 25 within the Gla domain, which is known to be juxtaposed to the Factor VIIIa light chain, and Tyr 69 (Nishimura, H., Takeya, H., Miyata, T., Suchiro, K., Okamura, T., Niho, Y. & Iwanaga, S. (1993) J Biol Chem 268, 24041-6) and Asn 92 (Hughes, P. E., Morgan, G., Rooney, E. K., Brownlee, G. G. & Handford, P. (1993) J Biol Chem 268, 17727-33). The putative binding region with 1811-1818 of Factor VIII is highlighted with dashed line. The putative phospholipids binding sites in the C1 and C2 domains of Factor VIIIa and the Gla domain of Factor IXa that are responsible for membrane binding are indicated (dark grey). As illustrated in FIG. 4B, positions of residues contributed from different loops of C2 domain resemble that of the 330-339 α-helix of Factor IXa.

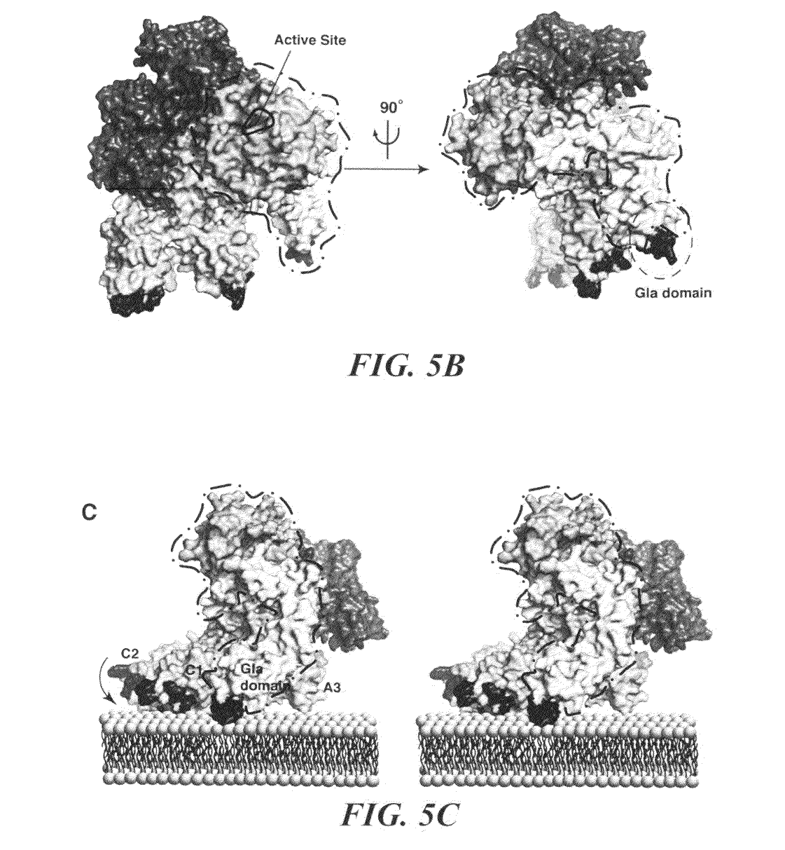

FIG. 5A shows a plot of the intermolecular energy, Eᵢₙₜₑᵣ (the sum of intermolecular van der Waals, electrostatic, and AIR energy terms) for 60 complex structures, after water refinement, as a function of their backbone rmsd from the lowest energy structure. Three main clusters, labeled “1,” “2,” and “3,” were obtained after analysis, using a 5 Å rmsd cut-off to distinguish the clusters. FIGS. 5B and 5C depict a model of the Factor IXa-Factor VIIIa complex including front and side views of the complex of Factor VIIIa (dark and light grey) and Factor IXa (outlined in solid line). Four putative membrane binding sites, including the bases of C1 and C2 domains, the A3 domain loop of Factor VIII and the Gla domain of Factor IXa, lie on the same plane and are darkly shaded (lower region of structures). The active site of Factor IXa is indicated (outlined in dashed line). Based on the model of the Factor IXa-Factor VIIIa complex and the x-ray crystal structure of Factor IXa, the A3 domain of Factor VIII is predicted to play a role in the interaction between the Factor IXa-Factor VIIIa complex and the phospholipid membrane.

### Representations of Structures

Structural information of B-domain deleted Factor VIII, or regions thereof (e.g., domain A1, A2, A3, C1, C2), and structural models of the present invention (including various structural models designed by computer-based methods described below) can be represented in many different ways. Particularly useful are electronic representations, as such representations allow rapid and convenient data manipulations and structural modifications. Electronic representations can be embedded in many different storage or memory media, frequently computer readable media. Examples include without limitations, computer random access memory (RAM), floppy disk, magnetic hard drive, magnetic tape (analog or digital), compact disk (CD), optical disk, CD-ROM, memory card, digital video disk (DVD), and others. The storage medium can be separate or part of a computer system. Such a computer system may be a dedicated, special purpose, or embedded system, such as a computer system that forms part of an X-ray crystallography system, or may be a general purpose computer (which may have data connection with other equipment such as a sensor device in an X-ray crystallographic system. In many cases, the information provided by such electronic representations can also be represented physically or visually in two or three dimensions, e.g., on paper, as a visual display (e.g., on a computer monitor as a two dimensional or pseudo-three dimensional image) or as a three dimensional physical model. Such physical representations can also be used, alone or in connection with electronic representations. Exemplary useful representations include, but are not limited to, the following:

Atomic Coordinate Representation

One type of representation is a list or table of atomic coordinates representing positions of particular atoms in a molecular structure, portions of a structure, or complex (e.g., a co-crystal). Such a representation may also include additional information, for example, information about occupancy of particular coordinates.

Energy Surface or Surface of Interaction Representation

Another representation is an energy surface representation, e.g., of an active site or other binding site, representing an energy surface for electronic and steric interactions. Such a representation may also include other features. An example is the inclusion of representation of a particular amino acid residue(s) or group(s) on a particular amino acid residue(s), e.g., a residue or group that can participate in H-bonding or ionic interaction.

Structural Representation

Still another representation is a structural representation, i.e., a physical representation or an electronic representation of such a physical representation. Such a structural representation includes representations of relative positions of particular features of a molecule or complex, often with linkage between structural features. For example, a structure can be represented in which all atoms are linked; atoms other than hydrogen are linked; backbone atoms, with or without representation of sidechain atoms that could participate in significant electronic interaction, are linked; among others. However, not all features need to be linked. For example, for structural representations of portions of a molecule or complex, structural features significant for that feature may be represented (e.g., atoms of amino acid residues that can have significant binding interation with a ligand at a binding site. Those amino acid residues may not be linked with each other.

A structural representation can also be a schematic representation. For example, a schematic representation can represent secondary and/or tertiary structure in a schematic manner. Within such a schematic representation of a polypeptide, a particular amino acid residue(s) or group(s) on a residue(s) can be included, e.g., conserved residues in a binding site, and/or residue(s) or group(s) that may interact with binding compounds.

### Uses of the Crystals and Atomic Structure Coordinates

The x-ray crystallographic structure of a biologically active human Factor VIII provides important structural information of Factor VIII, such as, for example, the domain organization, the metal binding sites and the surface features in this protein. In particular, the invention provides structural coordinates of atoms corresponding to various binding regions of Factor VIII, such as, for example, the low-density lipoprotein receptor-related protein (LRP) binding site (e.g., amino acids 484-509), heparin sulfate proteoglycans (HSPGs) binding site (e.g., amino acids 558-565), Factor IXa binding regions (e.g., amino acids 558-565, 707-712 or 1811-1819), and LRP/vWF/phospholipids (PL) binding region (e.g., amino acids 2303-2332).

Given the critical importance of Factor VIII to normal hemostasis in the blood coagulation cascade, the crystals of the invention, and particularly the atomic structure coordinates obtained therefrom, have a wide variety of uses including the design of improved therapies for hemophilia A. For example, the crystals described herein provides a useful tool for exploring the rich database of missense mutations that characterize many forms of hemophilia A and link them to functional abnormalities in vitro and in vivo. For example, the present invention allows detailed analysis of structure-function role of specific amino acids in Factor VIIIa in binding to the enzyme Factor IXa, activation of Factor VIII to Factor VIIIa by thrombin or factor Xa, binding of the substrate, factor X, and interaction of the complex with membrane surfaces. Understanding the structure-function relationship of the Factor IXa-Factor VIIIa complex and its interaction with membrane surfaces is critically important to detailed understanding of normal hemostasis within the context of the blood coagulation cascade.

The structures described herein can be used as a starting point in methods for modifying Factor VIII to improve its interaction with, for example, Factor IXa, vWF, or phospholipids membranes, resulting in modified Factor VIII with improved plasma half-life, improved functional activity (e.g., increased activation, or resistance to inactivation), reduced antigenicity or immunogenicity.

The structure coordinates described herein can also be used as phasing models for determining the crystal structures of Factor VIII homologs or analogs of unknown structure, as well as the structures of co-crystals of Factor VIII with ligands such as inhibitors, agonists, antagonists, and other molecules.

The structure described in herein can be used to design compounds that mimic Factor VIII cofactor activity. For example, mimetic antibodies can be designed based on the structure of Factor VIII described herein to substitute for Factor VIII cofactor activity. Such antibodies can be used as long-acting therapeutics for hemophilia.

In addition, the crystals and structure coordinates provided by the present invention are particularly useful for identifying compounds or molecules that modulate Factor VIII activity as an approach towards developing new therapeutic agents. In particular, the crystals and structural information are particularly useful in methods based on rational drug design.

A human Factor VIII structure suitable for various methods of these aspects of the invention includes structures defined by structural coordinates of atoms of human Factor VIII according to Table 2, +/−a root mean square deviation for alpha carbon atoms of less than 2 Angstroms, or selected coordinates thereof, as well as the models of Factor VIII homologs or analogs obtained by the methods of the invention.

In preferred embodiments, the methods of these aspects of the invention described above are computer-based methods. Various exemplary computational techniques suitable for the methods of these aspects of the invention are described below.

### Computational Techniques for In Silico Analysis and Design

Homology Modeling

Homology modeling is a method of applying structural coordinates of a polypeptide of known structure to the amino acid sequence of a polypeptide of unknown structure. This method is accomplished using a computer representation of the three dimensional structure of a polypeptide or polypeptide complex, the computer representation of amino acid sequences of the polypeptides with known and unknown structures, and standard computer representations of the structures of amino acids. Homology modeling generally involves (a) aligning the amino acid sequences of the polypeptides with and without known structure to match one or more homologous regions or amino acids; (b) modeling the structure of the matched one or more homologous regions or amino acids of the polypeptide of unknown structure on the corresponding regions of the known structure; and (d) determining a structural confirmation for the polypeptide of unknown structure which substantially preserves the structure of the matched homologous regions. Methods for matching homologous regions or amino acids are well known in the art. For example, the programs BLAST, gapped BLAST, BLASTN, PSI-BLAST and BLAST2 (provided by the National Center for Biotechnology Information) are widely used in the art for this purpose, and can align homologous regions of two amino acid sequences. These may be used with default parameters.

The above method is well known to those skilled in the art. (Greer (1985) Science 228:1055; Blundell et al. A(1988) Eur. J. Biochem., 172:513. An exemplary computer program that can be utilized for homology modeling by those skilled in the art is the Homology module in the Insight II modeling package distributed by Accelerys Inc.

Alignment of the amino acid sequence may be accomplished by first placing the computer representation of the amino acid sequence of a polypeptide with known structure above the amino acid sequence of the polypeptide of unknown structure. Amino acids in the sequences are then compared and groups of amino acids that are homologous (e.g., amino acid side chains that are similar in chemical nature—aliphatic, aromatic, polar, or charged) are grouped together. This method will detect conserved regions of the polypeptides and account for amino acid insertions or deletions.

Once the amino acid sequences of the polypeptides with known and unknown structures are aligned, the structures of the conserved amino acids in the computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions are to be assigned manually by either using standard peptide geometries or molecular simulation techniques, such as molecular dynamics. The final step in the process is accomplished by refining the entire structure using molecular dynamics and/or energy minimization. The homology modeling method is well known to those skilled in the art and has been practiced using different protein molecules. For example, the three dimensional structure of the polypeptide corresponding to the catalytic domain of a serine/threonine protein kinase, myosin light chain protein kinase, was homology modeled from the cAMP-dependent protein kinase catalytic subunit. (Knighton et al. (1992) Science 258:130-135.)

In addition, specific computer software is available in the art to evaluate compound deformation energy and electrostatic interactions. Examples of programs designed for such uses include: Gaussian 94, revision C (M. J. Frisch, Gaussian, Inc., Pittsburgh, Pa. C) 1995); AMBER, version 4.1 (P. A. Kollman, University of California at San Francisco, C) 1995) QUANTA/CHARMM (Molecular Simulations, Inc., San Diego, Calif. © 1995); Insight II/Discover (Molecular Simulations, Inc., San Diego, Calif. © 1995); DelPhi (Molecular Simulations, Inc., San Diego, Calif. © 1995); and AMSOL (Qunatum Chemistry Program Exchange, Indiana University). These programs may be implemented, for instance, using a Silicon Graphics workstation such as an Indigo² with “IMPACT” graphics. Other modem hardware systems and software packages will be known to those skilled in the art.

Molecular Replacement

Molecular replacement is a method of applying the X-ray diffraction data of a polypeptide of known structure to the X-ray diffraction data of a polypeptide of unknown sequence. This method can be utilized to define the phases describing the X-ray diffraction data of a polypeptide of unknown structure when only the amplitudes are known. X-PLOR is a commonly utilized computer software package used for molecular replacement. Brunger (1992) Nature 355:472-475. AMORE is another program used for molecular replacement. Navaza (1994) Acta Crystallogr. A50:157-163. Preferably, the resulting structure does not exhibit a root-mean-square deviation of more than 3 Å. Specific steps of molecular replacement are described below.

A goal of molecular replacement is to position the atomic coordinates of a structure model into the unit cell of Factor VIII crystal. A program such as X-PLOR can involve four steps. A first step can be to determine the number of molecules in the unit cell. A second step can involve rotating the structure model to define the orientation of the molecules in the unit cell. A third step can be to translate the structure model in three dimensions to correctly position the molecules in the unit cell. Once the amplitudes and phases of the X-ray diffraction data is determined, an R-factor can be calculated by comparing X-ray diffraction data calculated experimentally from the reference data set and calculated from the new data set. An R-factor between 30-50% indicates that the orientations of the atoms in the unit cell are reasonably determined by this method. A fourth step in the process can be to decrease the R-factor to roughly 25% or lower by refining the positioned structure model using iterative refinement techniques described herein and known to those or ordinary skill in the art.

Designing Mimetic Compounds of Factor VIII

In general, a mimetic compound of human Factor VIII may be designed by: (a) providing a selected human Factor VIII structure that is associated with a biological activity of human Factor VIII; (b) superimposing a three-dimensional structure of a compound on the selected human Factor VIII structure; and (c) modifying the three-dimensional structure of the compound such that the modified three-dimensional structure comprises a structural confirmation substantially mimicking the selected human Factor VIII structure.

A less biased approach involves computer algorithms for searching databases of three dimensional structures suitable compounds. By this method, one can generate compounds for which the bioactive conformation is heavily populated, i.e., compounds which are based on particularly biologically relevant conformations of the target protein. Algorithms for this purpose are implemented in programs such as Cast-3D (Chemical Abstracts Service), 3DB Unity (Tripos, Inc.), Quest-3D (Cambridge Crystallographic Data Center), and MACCS/ISIS-3D (Molecular Design Limited). These geometric searches can be augmented by steric searching, in which the size and shape requirements of the binding site are used to weed out hits that have prohibitive dimensions. Programs that may be used to synchronize the geometric and steric requirements in a search applied to the FRB of FRAP include CAVEAT (P. Bartlett, University of California, Berkeley), HOOK (MSI), ALADDIN (Daylight Software) and DOCK (I. D. Kuntz, University of California, San Francisco; see e.g. http://www.cmpharm.ucsf.edu/kuntz-/kuntz.html and references cited therein). All of these searching protocols may be used in conjunction with existing corporate databases, the Cambridge Structural Database, or available chemical databases from chemical suppliers.

In addition, mimetic compounds of Factor VIII may be developed from the bound conformation of Factor VIII by design, by searching databases for replacements of one or more structural segments of Factor VIII, or by enhancement of existing ligand-protein interactions (i.e., by replacing a component moiety of a ligand with a substitute moiety capable of greater interaction with the target protein, whether through accessible protein contact points or by extrusion of otherwise sequestered waters). Knowledge of the bound conformation of a ligand can suggest avenues for conformational restriction and replacement of atoms and/or bonds of Factor VIII.

Computer programs such as those described in the homology modeling section above can be used to superimpose a three-dimensional structure of a compound on the selected human Factor VIII structure; and to modify the three-dimensional structure of the compound such that the modified three-dimensional structure includes a structural confirmation substantially mimicking the selected human Factor VIII structure.

Modified compounds may be synthesized and the Factor VIII mimetic activity is tested by in vitro or in vivo methods known in the art.

In particular, the present invention contemplates mimetic antibodies of human Factor VIII designed by the methods described herein. For example, mimetic antibodies can be designed to mimic a binding activity of Factor VIII to Factor IXa and/or Factor X. Such mimetic antibodies may substitute for Factor VIII cofactor activity and could potentially be used as therapeutic agents for hemophilia.

Structure-Based Rational Drug Design

A particular aspect of the invention relates to computer-based rational drug design methods to identify candidate modulators of Factor VIII function that interact with human Factor VIII structures of the present invention.

Determination of the three-dimensional structure of B-domain deleted human Factor VIII provides important information about the binding sites of Factor VIII, particularly when comparisons are made with similar proteins, such as Factor Va, ceroluplasmin. This information may then be used for rational design, e.g., by computational techniques which identify possible binding ligands for the binding sites, by enabling linked-fragment approaches to drug design, and by enabling the identification and location of bound ligands using X-ray crystallographic analysis. The suitable techniques are discussed in detail, for example, by Walters et al. Drug Discovery Today, Vol. 3, No. 4, (1998), 160-178; and Abagyan, R.; Totrov, M. Curr. Opin. Chem. Biol. 2001, 5, 375-382. In particular, automated ligand-receptor docking programs is discussed, for example, by Jones et al. in Current Opinion in Biotechnology, Vol. 6, (1995), 652-656 and Halperin et al. Proteins 2002, 47, 409-443).

The aspects of the invention described herein which utilize the human Factor VIII structure in silico may be equally applied to both the human Factor VIII structure defined by coordinates of Table 2, or selected coordinates thereof, and the models of Factor VIII homologs or analogs obtained by other aspects of the invention. For rational drug design, the coordinates of atoms corresponding to one or more binding regions of Factor VIII are particularly useful. Such binding regions of Factor VIII include, but are not limited to, the LRP and Factor IXa binding region (e.g., amino acids 484-509), the HSPGs and Factor IXa binding region (e.g., amino acids 558-565), the Factor IXa binding regions (e.g., amino acids 707-712, and amino acids 1811-1819), and the LRP, vWF, PL binding region (e.g., amino acids 2303-2332).

Accordingly, the invention provides a computer-based method for the analysis of the interaction of a molecular structure with a human Factor VIII structure of the invention, which generally includes the steps of: providing selected coordinates of a human Factor VIII structure; providing a plurality of moieties to be fitted to said human Factor VIII structure; fitting the structure of each of the plurality of moieties to the human Factor VIII structure; selecting one or more moieties that fit into the selected structure; and, optionally, assembling the fitted one or more moieties into a single molecule to form a candidate modulator molecule.

The moieties suitable for the method of this aspect of the invention can be selected from the group consisting of molecular fragments, small molecules, ligands designed de novo, and compounds known to bind Factor VIII or modified compounds thereof.

In some embodiments, such moieties may be selected from publicly available databases include, for example: a) ACD from Molecular Designs Limited; b) NCI from National Cancer Institute; c) CCDC from Cambridge Crystallographic Data Center; d) CAST from Chemical Abstract Service; e) Derwent from Derwent Information Limited; f) Maybridge from Maybridge Chemical Company LTD; g) Aldrich from Aldrich Chemical Company; h) Directory of Natural Products from Chapman & Hall.

The availability of the structure of B-domain deleted human Factor VIII will allow the generation of highly predictive pharmacophore models for virtual library screening or compound design. The modeling software can be used to determine Factor VIII binding surfaces and to reveal features such as van der Waals contacts, electrostatic interactions, and/or hydrogen bonding opportunities. These binding surfaces can be used to model docking of ligands with Factor VIII, to arrive at pharmacophore hypotheses, and to design possible therapeutic compounds de novo. The term “pharmacophore” refers to a collection of chemical features and three-dimensional constraints that represent specific characteristics responsible for a ligand's activity. The pharmacophore includes surface-accessible features, hydrogen bond donors and acceptors, charged/ionizable groups, and/or hydrophobic patches, among other features.

A pharmacophore can be defined for the Factor VIII ternary complex that includes surface-accessible features, hydrogen bond donors and acceptors, charged/ionizable groups, and/or hydrophobic patches, among other features. These features can be weighted depending on their relative importance in conferring activity (see, e.g., Computer-Assisted Lead Finding and Optimization, Testra & Folkers, 1997).

Pharmacophores can be determined using software such as CATALYST (including HypoGen or HipHop, available from Molecular Stimulations Inc.), CERIUS2, or constructed by hand from a known conformation of a lead compound. The pharmacophore can be used to screen structural libraries, using a program such as CATALYST. The CLIX program (Davic & Lawrence, Proteins 12:31-41, 1992) can also be used, which searches for orientations of candidate molecules in structural databases that yield maximum spatial coincidence with chemical groups which interact with the receptor. The DISCO program (available from Tripos) uses a method of clique detection to identify common pharmacophoric features in each structure, produce optimally aligned structures, and extract the key features of the pharmacophore. The GASP program (available from Tripos) uses a genetic algorithm to automatically find pharmacophores with conformational flexibility.

The binding surface or pharmacophore of the Factor VIII ternary complex can be used to map favorable interaction positions for functional groups (e.g., protons, hydroxyl groups, amine groups, acidic groups, hydrophobic groups and/or divalent cations) or small molecule fragments. Compounds can then be designed de novo in which the relevant functional groups are located in the correct spatial relationship to interact with Factor VIII.

There are many de novo ligand design methods including:

1. LUDI (H.-J. Bohm, “The Computer Program LUDI: A New Method for the De Novo Design of Enzyme Inhibitors,” J. Comp. Aid. Molec. Design, 6, pp. 61-78 (1992)). LUDI is available from Molecular Simulations Incorporated, San Diego, Calif.
2. LEGEND (Y. Nishibata et al., Tetrahedron, 47, p. 8985 (1991)). LEGEND is available from Molecular Simulations Incorporated, San Diego, Calif.
3. LeapFrog (available from Tripos Associates, St. Louis, Mo.).
4. SPROUT (V. Gillet et al., “SPROUT: A Program for Structure Generation,” J. Comput. Aided Mol. Design, 7, pp. 127-153 (1993)). SPROUT is available from the University of Leeds, UK.

In order to provide a three-dimensional structure of moieties to be fitted to a human Factor VIII structure of the invention, the moiety structure may be modeled in three dimensions using commercially available software for this purpose or, if its crystal structure is available, the coordinates of the structure may be used to provide a representation of the compound for fitting to a human Factor VIII structure of the invention.

By “fitting”, it is meant determining by automatic, or semi-automatic means, interactions between at least one atom of a molecular structure and at least one atom of a human Factor VIII structure of the invention, and calculating the extent to which such an interaction is stable. Interactions include attraction and repulsion, brought about by charge, steric considerations and the like. Various computer-based methods for fitting are available in the art, for example, docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al., J. Mol. Biol. 1982, 161, 269-288, Makino et al., J. Comput. Chem. 1997, 18, 1812-1825), AUTODOCK (Goodsell et al., Proteins 1990, 8, 195-202, Morris et al., J. Comput. Chem. 1998, 19, 1639-1662.), FlexX, (Rarey et al., J. Mol. Biol. 1996, 261, 470-489) or ICM (Abagyan et al., J. Comput. Chem. 1994, 15, 488-506). This procedure can include computer fitting of a moiety to a human Factor VIII Structure to ascertain how well the shape and the chemical structure of the moiety will bind to human Factor VIII.

Also computer-assisted, manual examination of the structure of human Factor VIIIa may be performed. The use of programs such as GRID (Goodford, J. Med. Chem., 28, (1985), 849-857)—a program that determines probable interaction sites between molecules with various functional groups and an enzyme surface—may also be used to analyse the active site to predict, for example, the types of modifications which will alter the rate of catabolism of a substrate.

Computer programs can be employed to estimate the attraction, repulsion, and steric hindrance of the two binding partners.

Following the fitting of the molecular structures, a person of skill in the art may seek to use molecular modeling to determine to what extent the structures interact with each other (e.g., by hydrogen bonding, other non-covalent interactions, or by reaction to provide a covalent bond between parts of the structures) or the interaction of one structure with human Factor VIII is altered by the presence of another structure.

Once suitable moieties (such as, for example, chemical entities or fragments) have been selected, they can be designed or assembled into a single compound or complex. Assembly may be preceded by visual inspection of the relationship of the fragments to each other on the three-dimensional image displayed on a computer screen in relation to the structure coordinates of human serum albumin. This would be followed by manual model building using software such as Quanta or Sybyl (Tripos Associates, St. Louis, Mo.).

If more than one human Factor VIII region is characterized and a plurality of respective smaller moieties are designed or selected, a candidate modulator may be formed by linking the respective small moieties into a larger molecule, which maintains the relative positions and orientations of the respective smaller moieties at the respective binding regions. The candidate modulator may be formed as a real molecule or by computer modeling.

Useful programs to aid one of skill in the art in connecting the individual chemical entities or fragments include:

1. CAVEAT (P. A. Bartlett et al., “CAVEAT: A Program to Facilitate the Structure-Derived Design of Biologically Active Molecules,” in Molecular Recognition in Chemical and Biological Problems, Special Pub., Royal Chem. Soc., 78, pp. 182-196 (1989); G. Lauri and P. A. Bartlett, “CAVEAT: a Program to Facilitate the Design of Organic Molecules,” J. Comput. Aided Mol. Des., 8, pp. 51-66 (1994)). CAVEAT is available from the University of California, Berkeley, Calif.
2. 3D Database systems such as ISIS (MDL Information Systems, San Leandro, Calif.). This area is reviewed in Y. C. Martin. “3D Database Searching in Drug Design,” J. Med. Chem., 35, pp. 2145-2154 (1992).
3. HOOK (M. B. Eisen et al., “HOOK: A Program for Finding Novel Molecular Architectures that Satisfy the Chemical and Steric Requirements of a Macromolecule Binding Site,” Proteins: Struct., Funct. Genet., 19, pp. 199-221 (1994). HOOK is available from Molecular Simulations, San Diego, Calif.

Detailed structural information can then be obtained about the binding of the candidate modulator to human Factor VIII, and in the light of this information adjustments can be made to the structure or functionality of the candidate modulator, e.g., to alter its interaction with human Factor VIII. The above steps may be repeated and re-repeated as necessary.

A newly designed candidate modulator molecule may be obtained or synthesized and its interaction with human Factor VIII may be determined by binding assays. Various binding assays are well known in the art.

Such modulators may affect the interactions between Factor VIII and its binding partners, such as, LRP, Factor IXa, HSPGs, vWF, and PL, resulting in changed pharmacokinetics and functional activity for Factor VIII. Modulators of human Factor VIII identified by rational drug design may be developed as potential therapeutic agents for hemophilia.

Various molecular analysis and rational drug design techniques are further disclosed in, for example, U.S. Pat. Nos. 5,834,228, 5,939,528 and 5,856,116, as well as in PCT Application No. PCT/US98/16879, published as WO 99/09148, the teachings of all of which are hereby incorporated by reference.

The invention is illustrated by the following non-limiting examples.

### EXAMPLES

### Example 1

### Expression and Purification of B-Domain Deleted Factor VIII

The mature B-domain deleted human Factor VIII used for crystallization contains residues 1-740 (SEQ ID NO:1) that comprise the heavy chain (A1 and A2 domains), a short peptide linker (residues 741-754) (SEQ ID NO:3) and residues 1649-2332 (SEQ ID NO:2) that comprise the light chain (A3, C1 and C2 domains) (FIG. 1A). B-domain deleted recombinant Factor VIII (Wyeth) was prepared as previously described (Sandberg, H., Almstedt, A., Brandt, J., Castro, V. M., Gray, E., Holmquist, L., Lewin, M., Oswaldsson, U., Mikaelsson, M., Jankowski, M. A., Bond, M. & Scoble, H. A. (2001) Semin Hematol 38, 4-12; Eriksson, R. K., Fenge, C., Lindner-Olsson, E., Ljungqvist, C., Rosenquist, J., Smeds, A. L., ostlin, A., Charlebois, T., Leonard, M., Kelley, B. D. & Ljungqvist, A. (2001) Semin Hematol 38, 24-31) with the following modifications. Chinese hamster ovary cells were cultured in medium free of human serum albumin and purified by monoclonal antibody immunoaffinity chromatography was replaced with a peptide ligand affinity chromatography using TN8.2 Sepharose (Kelley, B. D., Tannatt, M., Magnusson, R., Hagelberg, S. & Booth, J. (2004) Biotechnol Bioeng 87, 400-12). The purified Factor VIII used for crystallization was obtained detergent-free following the anion exchange step used in the manufacturing process. Purified Factor VIII at a concentration of approximately 2.5 mg/ml in 50 mM histidine pH 6.3, 4 mM CaCl₂, 400 mM NaCl was stored at −80° C.

### Example 2

### Formation of B-Domain Deleted Factor VIII Crystals and Data Collection

Crystals were obtained by hanging drop vapor diffusion at 25° C. using the Hampton Screen (Hampton Research). The drop contained 1 μl of Factor VIII at 10 mg/ml mixed with 1 μl of reservoir solution. The optimal condition for crystallization was found to be 100 mM Tris-HCl (pH 8.5), 10% ethanol, and 7% PEG 3350 in the reservoir. All crystals were cryo-protected by sequential addition of 10%, 15% and finally 20% ethylene glycol (v/v) in the presence of the reservoir solution and flash frozen in liquid nitrogen prior to data collection. Factor VIII crystallized in a P4₁2₁2 space group (a=b=134.11 Å, c=349.760 Å and α=β=γ=90°) with one molecule per asymmetric unit (Table 1). The crystal contains an unusually high solvent content of 75%.

X-ray diffraction data of Factor VIII crystals were collected at the NE-CAT synchrotron beamline ID-24 of the Advanced Photon Source (APS) at Argonne National Laboratory. All xray data were processed using program HKL2000 (Otwinowski, Z. & Minor, W. (1997) in Methods Enzymol (Academic Press, New York), Vol. 276, pp. 307-326) (Table 1).

TABLE I
Statistics on diffraction data and structure refinement of B-domain deleted
human Factor VIII.

| Crystal | |
|---|---|
| Data Collection | |
| Data Collection Source | APS ID-24 |
| Wavelength (Å) | 0.97918 |
| Resolution (Å) | ∞-3.98 |
| Space group | P4₁2₁2 |
| Unit cell | a = b = 134.11 Å, c = 349.76 Å, |
| | α = β = γ = 90° |
| Redundancy (outer shell) | 13.2 (8.0) |
| No. of unique reflections (outer shell) | 27631 (2505) |
| Completeness (outer shell) (%) | 99.0 (92.3) |
| I/σ (overall/outer shell) | 27.2 (1.7) |
| R_{sym}^{a} (overall/outer shell) (%) | 13.0 (82.7) |
| Refinement | |
| Resolution (Å) | 50-3.98 |
| R_{crys}^{b} (%) | 25.56 |
| R_{free}^{c} (%) | 32.69 |
| R.m.s. deviations | |
| Bond lengths (Å) | 0.017 |
| Bond angles | 1.741 |

^{a}R_{sym} = Σ|I − <I>|/ΣI
^{b}R_{crys} = Σ∥F_{obs}| − |F_{calc}∥/Σ|F_{obs}|, where F_{obs} and F_{calc} are the observe and calculated structure factors, respectively.
^{c}R_{free} was calculated with 5% of the data excluded from the refinement calculation.

### Example 3

### Structure Determination and Refinement of Factor VIII

For structural determination using the molecular replacement method, a homology structure model of Factor VIII was constructed from the known primary sequence of Factor VIII (McCoy, A. J. (2007) Acta Crystallogr D Biol Crystallogr 63, 32-41). Using the template structures of Factor Vai (PDBID: 15DD) (Sandberg, H., Almstedt, A., Brandt, J., Castro, V. M., Gray, E., Holmquist, L., Lewin, M., Oswaldsson, U., Mikaelsson, M., Jankowski, M. A., Bond, M. & Scoble, H. A. (2001) Semin Hematol 38, 4-12) and ceruloplasmin (PDBID: 1KCW) (Adams, T. E., Hockin, M. F., Mann, K. G. & Everse, S. J. (2004) Proc Natl Acad Sci U SA 101, 8918-23), the structure of the A1 and A2 domains of Factor VIII were initially determined with AMoRe (Murshudov, G. N., Vagin, A. A. & Dodson, E. J. (1997) Acta Crystallogr D Biol Crystallogr 53, 240-55) using the structure of the A1 and A2 domains of the homology model. This yielded clear rotation function and translation function solutions. The A3 domain was then solved by AMoRe after fixing the solution of the A1 and A2 domains and using the A3 domain of ceroluplasmin as a search model (PDBID: 1KCW) (Adams, T. E., Hockin, M. F., Mann, K. G. & Everse, S. J. (2004) Proc Natl Acad Sci USA 101, 8918-23). After fixing all three A domains together, the positions of the C1 and C2 domains were determined with the program PHASER using a polyalanine model built from the high resolution structure of the C2 domain of Factor VIII (PDBID: ID7P) (Pratt, K. P., Shen, B. W., Takeshima, K., Davie, E. W., Fujikawa, K. & Stoddard, B. L. (1999) Nature 402, 439-442; Potterton, E., Briggs, P., Turkenburg, M. & Dodson, E. (2003) Acta Crystallogr D Biol Crystallogr 59, 1131-7). The structure was refined with several cycles of manual refitting and refinements using REFMAC of the CCP41 suite (Bihoreau, N., Pin, S., de Kersabiec, A. M., Vidot, F. & Fontaine-Aupart, M. P. (1994) Eur J Biochem 222, 41-8; Messerschmidt, A. & Huber, R. (1990) Eur J Biochem 187, 341-52). The R_{cryst} and R_{free} for the Factor VIII model were 25.70% and 33.06% respectively for data from 50-3.98 Å (Table 1).

### Example 4

### Structural Analysis of Factor VIII

Structural analysis was conducted by methods described above and those known in the art. Our model indicates that Factor VIII is a heterodimer consisting of the heavy chain (A1-A2 domains) and light chain (A3-C1-C2 domains). Several regions within the structure are poorly ordered and were not modeled, including residues 17-43, 334-376, and 714-754 within the heavy chain, and residues 1649-1690 and 1714-1724 of the light chain.

The overall structure of Factor VIII can be described as a triangular heterotrimer of the A domains stacked on two smaller globular C domains (FIG. 1B). This structure closely resembles that of inactivated bovine Factor Va, Factor Vai (Adams, T. E., Hockin, M. F., Mann, K. G. & Everse, S. J. (2004) Proc Natl Acad Sci USA 101, 8918-23), except that B-domain deleted Factor VIII also contains the A2 domain. The A1, A2 and A3 domains each consist of two connected β-barrels that resemble the fold of a typical cupredoxin-type domain (Zaitseva, I., Zaitsev, V., Card, G., Moshkov, K., Bax, B., Ralph, A. & Lindley, P. (1996) J. Biol. Inorg. Chem. 1, 15). All three A domains share high structural homology with each other (average rmsd 1.40 Å). The C1 and C2 domains are defined by a distorted β-barrel and share structural homology (rmsd 1.09 Å). The Factor VIII C1 domain is homologous to the C1 domain of Factor Va (rmsd 1.04 Å) whereas the Factor VIII C2 domain is homologous to the C2 domain of Factor Va (rmsd 0.93 Å) and is nearly identical with the Factor VIII C2 domain determined at 1.5 Å resolution (rmsd 0.73 Å) (Pratt, K. P., Shen, B. W., Takeshima, K., Davie, E. W., Fujikawa, K. & Stoddard, B. L. (1999) Nature 402, 439-442) (FIG. 1C). The final model includes 630 residues of the heavy chain, 631 residues of light chain, two Cu²⁺ ions, one Ca²⁺, and three carbohydrate moieties. The coordinates of the Factor VIII structural model is shown in Table 2.

Factor VIII is a copper binding protein (Bihoreau, N., Pin, S., de Kersabiec, A. M., Vidot, F. & Fontaine-Aupart, M. P. (1994) Eur J Biochem 222, 41-8), and we identified two copper ions and their binding sites internally within the A1 domain and the A3 domain. These are prototypic copper binding sites, with nitrogen and sulhydryl ligands (Messerschmidt, A. & Huber, R. (1990) Eur J Biochem 187, 341-52). In the A3 domain, the copper ion is liganded by His 1954, Cys 2000, His 2005 whereas the copper binding site in the A1 domain is defined by His 267, His 315 and Cys 310 (FIG. 1D). These copper ions are not located at the domain interface, thus indicating that their role in enhancing light chain-heavy chain interaction is indirect (Wakabayashi, H., Koszelak, M. E., Mastri, M. & Fay, P. J. (2001) Biochemistry 40, 10293-300).

A single calcium binding site was located in the A1 domain. This site is defined by carboxyl groups of Glu 110, Asp 116, Asp 126, Asp 125 and the carbonyl 0 of Lys 107 and Glu 122 (FIG. 1D).)

Oligosaccharides was observed linked to Asn 239 in the A1 domain, Asn 1810 in the A3 domain and Asn 2118 in the C1 domain.

The three A domains form a triangular heterotrimer around a pseudo-three-fold symmetry where A1 and A3 domains serve as the base and interact with the C2 and C1 domains respectively (FIG. 2A). The front surface of the triangular heterotrimer of the A domains is planar whereas the back surface is dominated by three protrusions, formed by large loops from each domain, that create a deep groove at the center of the three domains (FIG. 2A). The C1 and C2 domains are adjacent at the base of the triangular heterotrimer. Each C domain projects three β-hairpin loops containing hydrophobic and basic residues toward the same plane. These loops likely contribute to the interaction of Factor VIII with the phospholipid bilayer (FIGS. 2B and 2C).

Although the C2 domain is connected to the C1 domain and located adjacent to the A1 domain, there are few direct contacts of the C2 with either domain (FIG. 2D). The interface between the C2 and A1 domains only buries 371 Å² of solvent accessible surface area. Only Arg 121 from the A1 domain is in sufficient proximity to form hydrogen bonds with the backbone carbonyl of Leu 2302 and the side chain of Gln 2266, and only Glu 122 is capable of an electrostatic interaction with Lys 2239 (FIG. 2D, left inset). The only close interactions between the C1 and C2 domains are observed within the loop that connects the two domains (residues 2168 to 2175), and between Met 2176 and Thr 2023, Val 2294 and Ser 2029 (FIG. 2D, right inset). These contacts cover a limited accessible surface area (371 Å²). Together, these observations strongly suggest the potential flexibility of the C2 domain and are in agreement the concept that the C2 domain undergoes conformational changes upon proteolysis within the light chain, resulting in enhanced affinity of Factor VIIIa for anionic phospholipids surfaces (Saenko, E. L., Scandella, D., Yakhyaev, A. V. & Greco, N. J. (1998) J Biol Chem 273, 27918-26).

Factor VIII and Factor V are procofactors that show approximately 40% sequence similarity and a parallel domain arrangement in their primary structure (Kane, W. H. & Davie, E. W. (1986) Proc Natl Acad Sci USA 83, 6800-4). Both cofactors are activated to their active cofactor forms, Factor VIIIa and Factor Va, by thrombin-mediated limited proteolysis (Pittman, D. D. & Kaufman, R. J. (1988) Proc Natl Acad Sci USA 85, 2429-33; Nesheim, M. E. & Mann, K. G. (1979) J Biol Chem 254, 1326-34). Both cofactors are inactivated by activated protein C-mediated limited proteolysis to yield Factor VIIIai and Factor Vai. The three dimensional structure of Factor V has not been solved, thus precluding direct comparison with our current structure of human B-domain deleted Factor VIII. However, a high resolution x-ray crystal structure of activated protein C-inactivated bovine Factor Va, Factor Vai, allows partial comparison since activation and subsequent inactivation of Factor V is associated with the removal of the B-domain and the A2 domain (Adams, T. E., Hockin, M. F., Mann, K. G. & Everse, S. J. (2004) Proc Natl Acad Sci USA 101, 8918-23). The spatial arrangement of the A1 and A3 domains in B-domain deleted Factor VIII are nearly identical to that of Factor Vai, while the C1 and C2 domains show slightly different conformations when comparing our Factor VIII structure and that of Factor Vai. Despite the differences in the C1 and C2 domains, structures of Factor VIII and Factor Vai can be superimposed with a rmsd of 1.54 Å for the carbons of the 524 residues that span the A1, A2, C1 and C2 domains (FIG. 3A).

### Example 5

### Implications for the Activation of Factor VIII to Factor VIIIa

The activation of Factor VIII by thrombin requires cleavage of a peptide bond in the A2 domain, after Arg 372, and the removal of the B-domain linked to the 41 residue N-terminal region of the A3 domain, typically referred to as the a3 acidic region comprising residues 1649-1689, by thrombin cleavage after Arg 1689. This exposes the Factor VIII surfaces that are important for Factor IXa binding. Activation is also associated with but does not require cleavage after arginine 740 (Pittman, D. D. & Kaufman, R. J. (1988) Proc Natl Acad Sci USA 85, 2429-33). In B-domain deleted Factor VIII, conversion of Factor VIII to Factor VIIIa requires cleavage at arginine 1689 to remove the acidic a3 region adjacent to the A3 domain as well as cleavage after Arg 372. These regions are located on the front surface of Factor VIII (FIG. 1A and FIG. 3B). Thus, it appears that the B-domain and the a3 acidic region are positioned on one face of the triangular heterotrimeric A domains of Factor VIII and obstruct a functionally significant surface on the A2 and A3 domains (FIG. 3B). This putative B-domain interaction surface on Factor VIII includes all of the suggested Factor IXa binding regions previously described, including residues 558-565, 707-712 and 1811-1819 (Fay, P. J., Beattie, T., Huggins, C. F. & Regan, L. M. (1994) J Biol Chem 269, 20522-7; Jenkins, P. V., Dill, J. L., Zhou, Q. & Fay, P. J. (2004) Biochemistry 43, 5094-101; Lenting, P. J., van de Loo, J. W., Donath, M. J., van Mourik, J. A. & Mertens, K. (1996) J Biol Chem 271, 1935-40).

### Example 6

### Structural Model of the Complex of Factor VIII and Factor IXa

The assembly of the Factor IXa-Factor VIIIa complex involves the binding of Factor VIIIa and Factor IXa on phospholipid membrane surfaces in the presence of calcium ions. Based upon homology modeling (Autin, L., Miteva, M. A., Lee, W. H., Mertens, K., Radtke, K. P. & Villoutreix, B. O. (2005) J Thromb Haemost 3, 2044-56), the analysis of naturally occurring hemophilia A and B mutations or mutations introduced by site-specific mutagenesis (Mannucci, P. M. & Tuddenham, E. G. (2001) N Engl J Med 344, 1773-9; Jenkins, P. V., Dill, J. L., Zhou, Q. & Fay, P. J. (2004) Biochemistry 43, 5094-101; Nishimura, H., Takeya, H., Miyata, T., Suchiro, K., Okamura, T., Niho, Y. & Iwanaga, S. (1993) J Biol Chem 268, 24041-6; Hughes, P. E., Morgan, G., Rooney, E. K., Brownlee, G. G. & Handford, P. (1993) J Biol Chem 268, 17727-33), cross-linking studies (Blostein, M. D., Furie, B. C., Rajotte, I. & Furie, B. (2003) J Biol Chem 278, 31297-302) and inhibition with synthetic peptides (Lenting, P. J., van de Loo, J. W., Donath, M. J., van Mourik, J. A. & Mertens, K. (1996) J Biol Chem 271, 1935-40), the Factor VIIIa binding surface for Factor IXa is thought to involve the A2 and A3 domains that interact with multiple domains on Factor IXa. The A3 domain of the light chain contains a high affinity binding site (Kd˜2-15 nM) for Factor IXa (Lenting, P. J., Donath, M. J., van Mourik, J. A. & Mertens, K. (1994) J Biol Chem 269, 7150-5). Inhibition studies with synthetic peptides directed against the A3 domain have located this site to residues 1811-1818 (Lenting, P. J., van de Loo, J. W., Donath, M. J., van Mourik, J. A. & Mertens, K. (1996) J Biol Chem 271, 1935-40). Potential Factor IXa binding sites on the A2 domain include residues 558-565 (Fay, P. J., Beattie, T., Huggins, C. F. & Regan, L. M. (1994) J Biol Chem 269, 20522-7) and the region around Asp 712 (Jenkins, P. V., Dill, J. L., Zhou, Q. & Fay, P. J. (2004) Biochemistry 43, 5094-101). We have mapped these putative binding regions onto our structure of Factor VIII (FIG. 4A). All three binding regions are solvent-exposed on the front surface of the molecule and surround a portion of the interface between the A2 and A3 domains. One of these binding regions, residues 558-565, bound to the C2 domain of a symmetry-related molecule in the crystal. The interaction involves mainly hydrophobic and basic residues (e.g., Arg 2215, Lys 2249, Leu 2251) from the β-hairpin loops located at the bottom of the C2 domain. Despite the fact that these interacting residues are located on different loops of the C2 domain, their orientations closely resemble those in the α-helix region 330-339 in Factor fxa (FIG. 4B) (Bajaj, S. P., Schmidt, A. E., Mathur, A., Padmanabhan, K., Zhong, D., Mastri, M. & Fay, P. J. (2001) J Biol Chem 276, 16302-9). This crystal contact may be mimicking the interaction between Factor VIIIa and the 330-339 helix of Factor IXa.

The absence of B-domain and the disorder of the loops containing the cleavage sites necessary for Factor VIII activation suggest that the our current structural model resembles the covalent structure of Factor VIIIa. Human Factor IXa was constructed by homology modeling using the x-ray structure of porcine Factor IXa (PDB ID: 1PFX.pdb) and the program SWISS-MODEL (McCoy, A. J. (2007) Acta Crystallogr D Biol Crystallogr 63, 32-41; Wakabayashi, H., Koszelak, M. E., Mastri, M. & Fay, P. J. (2001) Biochemistry 40, 10293-300). The structure of Factor VIIIa was docked with Factor IXa using the program HADDOCK (Saenko, E. L., Scandella, D., Yakhyaev, A. V. & Greco, N. J. (1998) J Biol Chem 273, 27918-26). In this approach, residues previously reported to be important for Factor VIIIa-Factor IXa interactions were defined as the ambiguous interaction constraints. Initially, 600 structures for the complex were generated by docking Factor VIII and Factor IXa as rigid bodies during iterations of energy minimization and 150 of those structures were refined and analyzed by using the default settings.

In particular, we have constructed a model of the Factor IXa-Factor VIIIa complex using our Factor VIII structure and the x-ray crystal structure of porcine Factor IXa backbone using as constraints: (1) residues 558-565 of Factor VIII interact with the 330-339 helix of Factor IXa; (2) 707-712 of Factor VIIIa binds to Factor IXa residues 301-303; (3) residues 1811-1819 of Factor VIII interact with the light chain of Factor IXa (Lenting, P. J., van de Loo, J. W., Donath, M. J., van Mourik, J. A. & Mertens, K. (1996) J Biol Chem 271, 1935-40); (4) Phe 25 in the Gla domain of Factor IX is juxtaposed with the light chain of Factor VIII (Blostein, M. D., Furie, B. C., Rajotte, I. & Furie, B. (2003) J Biol Chem 278, 31297-302); and (5) the Gla domain of Factor IXa is situated within the phospholipid membrane, forming non-covalent interactions between the phosphoserine head group and fatty acid chains of the phospholipid bilayer and the hydrophobic patch and the Gla residues within the Gla domain of Factor LXa (Freedman, S. J., Blostein, M. D., Baleja, J. D., Jacobs, M., Furie, B. C. & Furie, B. (1996) Journal of Biological Chemistry 271, 16227-16236; Huang, M., Rigby, A. C., Morelli, X., Grant, M. A., Huang, G., Furie, B., Seaton, B. & Furie, B. C. (2003) Nature Structural Biology 10, 751-756) (FIG. 4A).

The model of the Factor IXa-Factor VIIIa complex illustrates that the light chain of Factor IXa, which includes the phospholipid-binding Gla domain, is wrapped across the side of the A3 domain and oriented almost perpendicularly to the Factor VIII molecule, and is distally located from the membrane-binding interface of the C2 domain, which is important for Factor VIIIa interaction with the membrane (FIG. 5B). This orientation of the Factor VIII light chain and Factor IX Gla domain suggest the C2 domain of Factor VIII has to undergo a significant conformational change in order to orient its membrane binding surfaces in the same direction as the Gla domain of Factor IXa. In fact, in addition to the observation that C2 domain must undergo a conformational change upon activation, the 15 Å resolution cryo-electron microscopy structure of Factor VIII bound to phospholipids shows that the A domains are inclined at an angle of 60-65° to the membrane surface, with the A3 domain positioned close to the membrane surface. Furthermore, Factor IXa has been shown to orient with the long size of the molecule perpendicularly to the membrane, the Gla domain proximal to the membrane with the active site positioned more than 70 Å above the surface (Mutucumarana, V. P., Duffy, E. J., Lollar, P. & Johnson, A. E. (1992) J Biol Chem 267, 17012-21). We suggest that upon binding to Factor IXa, the C2 domain of the phospholipid-bound Factor VIIIa undergoes a significant conformational change that alters the orientation of the Factor VIIIa from an upright position to a bent position, in order for the Gla domain of the bound Factor IXa to interact with the phospholipids membrane simultaneously. The active site of Factor IXa in this Factor IXa-Factor VIIIa complex model is positioned on the top of the complex facing into the solution, with an approximate distance of 75-80 Å above the membrane surface.

We have identified the four putative phospholipid binding sites in the Factor IXa-Factor VIIIa complex. Within the context of the domain organization of C2 in the Factor IXa-Factor VIII complex, Arg 2215 and Lys 2249 play a special role in phospholipids binding. The hairpin loops in the Factor VIII C1 domain, including Lys 2092, Arg 2090, Arg 2169, likely play a similar role. In addition, a well-defined loop extends downward from the backside of the Factor VIII A3 domain. This loop, which includes a β-turn, is held together by Cys 1899 and Cys 1903, and thrusts Arg 1900 into a favorable position for electrostatical interaction with acidic phospholipid headgroups. The polypeptide backbone of this loop is structurally identical to and co-planar with the omega loop of the Gla domain of Factor IXa. Using the prothrombin Gla domain as a prototype, we have previously established that the phosphoserine head group in lysophosphatidylserine interacts with conserved residues within the Gla domain of vitamin K-dependent proteins, including Factor IX (Huang, M., Rigby, A. C., Morelli, X., Grant, M. A., Huang, G., Furie, B., Seaton, B. & Furie, B. C. (2003) Nature Structural Biology 10, 751-756). Trp 4 within the omega loop of the Gla domain of prothrombin is located 5 to 7 Å below the membrane surface in the interfacial membrane region (Falls, L. A., Furie, B. C., Jacobs, M., Furie, B. & Rigby, A. C. (2001) J Biol Chem 276, 23895-902). Based on this observation, the Factor IXa in this model was similarly positioned.

### Example 7

### Comparison with a Structure of a Heterodimer Form of Factor VIII

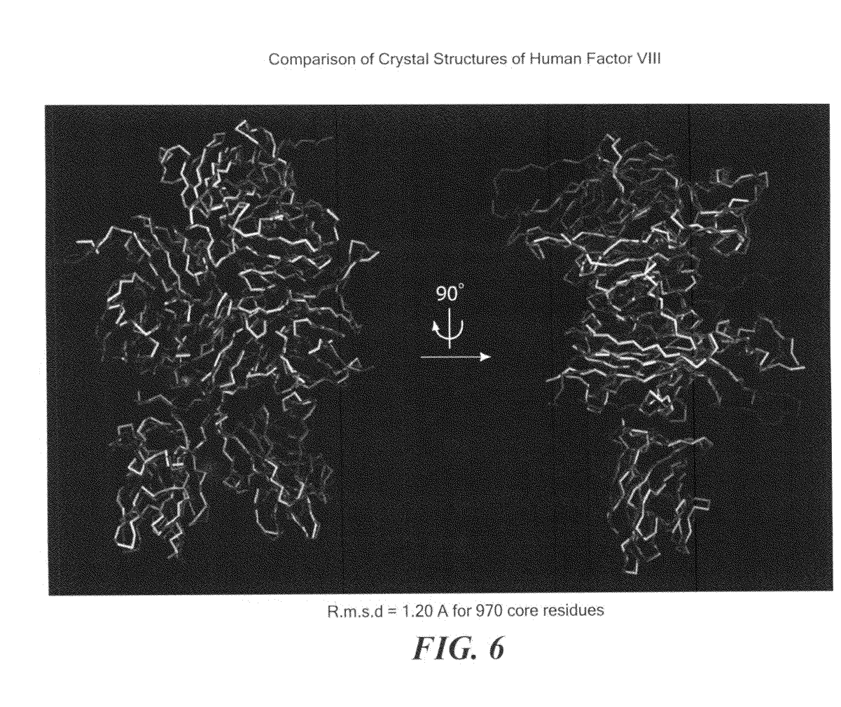

After the priority date of the present application, a crystal structure of a recombinant form of Factor VIII which consists of a heterodimer of peptides, respectively containing the A1-A2 and A3-C1-C2 domains, was disclosed on Apr. 15, 2008 (PDB ID: 2R7E). See Shen B. W. et al. “The tertiary structure and domain organization of coagulation Factor VIII,” Blood, 2008 Feb. 1; 111(3):1240-7, Epub 2007 Oct. 26. Overlaid of our B domain-deleted FVIII crystal structure (PDB ID: 3CDZ) and 2R7E shows that both structures are maintained in similar conformations with a few exceptions (FIG. 6).

Firstly, the loop formed by residues 558 to 565 in 3CDZ is solvent-exposed and appears to be accessible for the interaction with FIXa. In contrast, the model of 2R7E contains extra residues between amino acids 360-376 and 715-725, which form two large extensions and bury the 558-565 loop. Importantly, the extension formed by residues 360 to 376 contains the activating cleavage site (Arg 372). Cleavage at this position during FVIII activation may help to expose the 558-565 loop for FIXa binding. The difference between the two structures also suggests that this loop is flexible and may adopt different conformations upon binding with FIXa. Furthermore, the two extensions in 2R7E appear to be more ordered than those in 3CDZ because of the presence of B-domain regions in 2R7E. Therefore, the burial of the 558-568 loop may be a feature of the full length FVIII before activation and removal of the B-domain.

Secondly, residues 1712 to 1725 are disordered in 3CDZ but they form an extended loop in 2R7E. This loop is comprised mostly of basic and hydrophobic residues and is oriented in a similar manner as the other A3 domain loop (amino acids 1895-1907). Based on the proposed orientation of our FVIIIa:FIXa complex model, this region of the A3 domain of FVIII is also likely to interact with the acidic phospholipids membrane.

### Example 8

### Electrostatic Surface Potential of Factor VIII

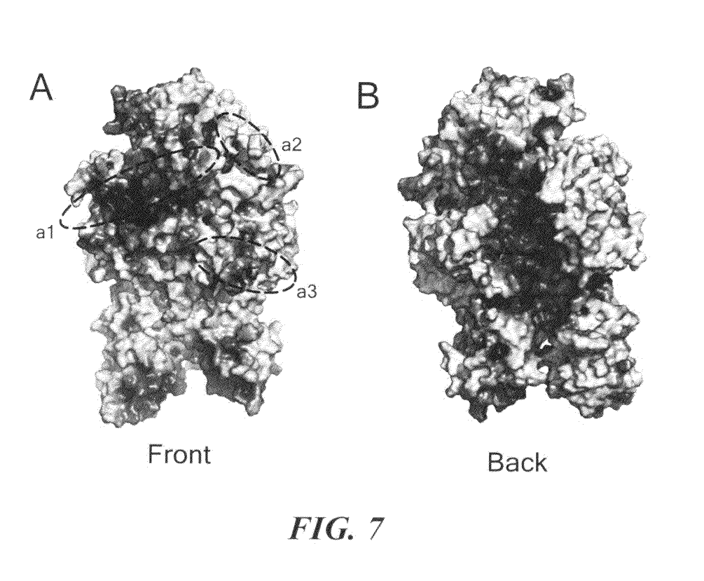

Exemplary electrostatic surface potential of Factor VIII is illustrated in FIG. 7. As shown, FVIII is highly charged on the surface. In addition to the positively charged bottoms of the C1 and C2 domains, the solvent-accessible surface near the interface between A1 and A2 domain is also highly positively charged. The acidic a1 peptide (337-372) is disordered and not modeled in our structure, suggesting that it may not interact strongly with this basic region in FVIII. However, based on the connecting regions of the peptide, it is expected to position near this region and may have a neutralizing effect. Without wishing to be bound by any particular theories, it is contemplated that the proteolytic cleavage at Arg 372 during activation of FVIII may reorient the acidic peptide to this region, resulting in both retention and reorientation of the A2 domain.

As also shown in FIG. 7, a deep groove formed by the large protrusions of A1, A2 and A3 domains is extensively negatively charged. Without wishing to be bound by any particular theories, it is contemplated that such striking property may cause strong repulsion between the domains and is responsible for the spontaneous dissociation of the A2 domain after FVIII activation. Interestingly, the binding interfaces between the A2 domain and the light chain and A1 domain contain large number of histidine residues. Previous studies have shown that the association between the A2 domain and A1/A3-C1-C2 dimer in FVIIIa is more stable at pH 6.0 (Lollar et al. (1990) “pH-dependent denaturation of thrombin-activated porcine Factor VIII,” J. Biol. Chem. 265, 1688-1692; Lamphear, B. J. et al. (1992) “Factor IXa enhances reconstitution of Factor VIIIa from isolated A2 subunit and A1/A3-C1-C2 dimer,” J. Biol. Chem. 267, 3725-3730). Without wishing to be bound by any theories, it is contemplated that the neutral property of histidine residues at pH 6.0 may play a role in the retention of A2 domain and that electrostatic potential plays an important role in the dissociation of A2 domain.

TABLE 2
Exemplary structural coordinates of B-domain-deleted human Factor VIII

| REMARK | 1 | | | |
|---|---|---|---|---|
| REMARK | 2 | | | |
| REMARK | 2 | RESOLUTION. 3.98 ANGSTROMS. | | |
| REMARK | 3 | | | |
| REMARK | 3 | REFINEMENT. | | |
| REMARK | 3 | PROGRAM : REFMAC 5.3.0037 | | |
| REMARK | 3 | AUTHORS : MURSHUDOV, VAGIN, DODSON | | |
| REMARK | 3 | | | |
| REMARK | 3 | REFINEMENT TARGET : MAXIMUM LIKELIHOOD | | |
| REMARK | 3 | | | |
| REMARK | 3 | DATA USED IN REFINEMENT. | | |
| REMARK | 3 | RESOLUTION RANGE HIGH | (ANGSTROMS) : | 3.98 |
| REMARK | 3 | RESOLUTION RANGE LOW | (ANGSTROMS) : | 50.00 |
| REMARK | 3 | DATA CUTOFF | (SIGMA(F)) : | 0.000 |
| REMARK | 3 | COMPLETENESS FOR RANGE | (%) : | 98.5 |
| REMARK | 3 | NUMBER OF REFLECTIONS | : | 26444 |
| REMARK | 3 | | | |

| REMARK | 3 | FIT TO DATA USED IN REFINEMENT. | | |
|---|---|---|---|---|
| REMARK | 3 | CROSS-VALIDATION METHOD | : | THROUGHOUT |
| REMARK | 3 | FREE R VALUE TEST SET SELECTION | : | RANDOM |

| | | | | |
|---|---|---|---|---|
| REMARK | 3 | R VALUE | (WORKING + TEST SET) : | 0.259 |
| REMARK | 3 | R VALUE | (WORKING SET) : | 0.256 |

| | | | | |
|---|---|---|---|---|
| REMARK | 3 | FREE R VALUE | : | 0.327 |

| | | | | |
|---|---|---|---|---|
| REMARK | 3 | FREE R VALUE TEST SET SIZE | (%) : | 5.000 |
| REMARK | 3 | FREE R VALUE TEST SET COUNT | : | 1399 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | : | 20 |
| REMARK | 3 | BIN RESOLUTION RANGE HIGH | : | 3.98 |
| REMARK | 3 | BIN RESOLUTION RANGE LOW | : | 4.08 |

| | | | | |
|---|---|---|---|---|
| REMARK | 3 | REFLECTION IN BIN | (WORKING SET) : | 1648 |
| REMARK | 3 | BIN COMPLETENESS | (WORKING + TEST) (%) : | 85.98 |
| REMARK | 3 | BIN R VALUE | (WORKING SET) : | 0.4140 |

| | | | | |
|---|---|---|---|---|
| REMARK | 3 | BIN FREE R VALUE SET COUNT | : | 87 |
| REMARK | 3 | BIN FREE R VALUE | : | 0.4550 |
| REMARK | 3 | | | |

| REMARK | 3 | NUMBER OF NON-HYDROGEN ATOMS USED IN REFINEMENT. | | |
|---|---|---|---|---|
| REMARK | 3 | ALL ATOMS : 10317 | | |
| REMARK | 3 | | | |
| REMARK | 3 | B VALUES. | | |
| REMARK | 3 | FROM WILSON PLOT | (A**2) : | NULL |
| REMARK | 3 | MEAN B VALUE | (OVERALL, A**2) : | 199.23 |

| REMARK | 3 | OVERALL ANISOTROPIC B VALUE. | | |
|---|---|---|---|---|
| REMARK | 3 | B11 (A**2) : 4.46000 | | |
| REMARK | 3 | B22 (A**2) : 4.46000 | | |
| REMARK | 3 | B33 (A**2) : −8.92000 | | |
| REMARK | 3 | B12 (A**2) : 0.00000 | | |
| REMARK | 3 | B13 (A**2) : 0.00000 | | |
| REMARK | 3 | B23 (A**2) : 0.00000 | | |
| REMARK | 3 | | | |
| REMARK | 3 | ESTIMATED OVERALL COORDINATE ERROR. | | |
| REMARK | 3 | ESU BASED ON R VALUE | (A): | NULL |
| REMARK | 3 | ESU BASED ON FREE R VALUE | (A): | 0.831 |
| REMARK | 3 | ESU BASED ON MAXIMUM LIKELIHOOD | (A): | 0.728 |
| REMARK | 3 | ESU FOR B VALUES BASED ON MAXIMUM LIKELIHOOD | (A**2): | 123.639 |
| REMARK | 3 | | | |

| | | | |
|---|---|---|---|
| REMARK | 3 | CORRELATION COEFFICIENTS. | |
| REMARK | 3 | CORRELATION COEFFICIENT FO-FC | : 0.925 |
| REMARK | 3 | CORRELATION COEFFICIENT FO-FC FREE | : 0.889 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 10617 ; | 0.014 ; | 0.022 |
| REMARK | 3 | BOND LENGTHS OTHERS | (A): | NULL ; | NULL ; | NULL |
| REMARK | 3 | BOND ANGLES REFINED ATOMS | (DEGREES): | 14405 ; | 1.735 ; | 1.951 |
| REMARK | 3 | BOND ANGLES OTHERS | (DEGREES): | NULL ; | NULL ; | NULL |
| REMARK | 3 | TORSION ANGLES, PERIOD 1 | (DEGREES): | 1255 ; | 9.793 ; | 5.000 |
| REMARK | 3 | TORSION ANGLES, PERIOD 2 | (DEGREES): | 495 ; | 37.797 ; | 23.515 |
| REMARK | 3 | TORSION ANGLES, PERIOD 3 | (DEGREES): | 1767 ; | 24.514 ; | 15.000 |
| REMARK | 3 | TORSION ANGLES, PERIOD 4 | (DEGREES): | 58 ; | 18.451 ; | 15.000 |
| REMARK | 3 | CHIRAL-CENTER RESTRAINTS | (A**3): | 1551 ; | 0.123 ; | 0.200 |
| REMARK | 3 | GENERAL PLANES REFINED ATOMS | (A): | 8038 ; | 0.004 ; | 0.020 |
| REMARK | 3 | GENERAL PLANES OTHERS | (A): | NULL ; | NULL ; | NULL |
| REMARK | 3 | NON-BONDED CONTACTS REFINED ATOMS | (A): | 5632 ; | 0.268 ; | 0.200 |
| REMARK | 3 | NON-BONDED CONTACTS OTHERS | (A): | NULL ; | NULL ; | NULL |
| REMARK | 3 | NON-BONDED TORSION REFINED ATOMS | (A): | 6731 ; | 0.322 ; | 0.200 |
| REMARK | 3 | NON-BONDED TORSION OTHERS | (A): | NULL ; | NULL ; | NULL |
| REMARK | 3 | H-BOND (X...Y) REFINED ATOMS | (A): | 336 ; | 0.215 ; | 0.200 |
| REMARK | 3 | H-BOND (X...Y) OTHERS | (A): | NULL ; | NULL ; | NULL |
| REMARK | 3 | POTENTIAL METAL-ION REFINED ATOMS | (A): | 1 ; | 0.021 ; | 0.200 |
| REMARK | 3 | POTENTIAL METAL-ION OTHERS | (A): | NULL ; | NULL ; | NULL |
| REMARK | 3 | SYMMETRY VDW REFINED ATOMS | (A): | 37 ; | 0.201 ; | 0.200 |
| REMARK | 3 | SYMMETRY VDW OTHERS | (A): | NULL ; | NULL ; | NULL |
| REMARK | 3 | SYMMETRY H-BOND REFINED ATOMS | (A): | 2 ; | 0.121 ; | 0.200 |
| REMARK | 3 | SYMMETRY H-BOND OTHERS | (A): | NULL ; | NULL ; | NULL |
| REMARK | 3 | SYMMETRY METAL-ION REFINED ATOMS | (A): | NULL ; | NULL ; | NULL |
| REMARK | 3 | SYMMETRY METAL-ION OTHERS | (A): | NULL ; | NULL ; | NULL |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2): | 6451 ; | 0.415 ; | 1.500 |
| REMARK | 3 | MAIN-CHAIN BOND OTHER ATOMS | (A**2): | NULL ; | NULL ; | NULL |
| REMARK | 3 | MAIN-CHAIN ANGLE REFINED ATOMS | (A**2): | 10173 ; | 0.751 ; | 2.000 |
| REMARK | 3 | SIDE-CHAIN BOND REFINED ATOMS | (A**2): | 4826 ; | 0.712 ; | 3.000 |
| REMARK | 3 | SIDE-CHAIN ANGLE REFINED ATOMS | (A**2): | 4232 ; | 1.208 ; | 4.500 |
| REMARK | 3 | | | | | |
| REMARK | 3 | ANISOTROPIC THERMAL FACTOR RESTRAINTS. | | COUNT | RMS | WEIGHT |
| REMARK | 3 | RIGID-BOND RESTRAINTS | (A**2): | NULL ; | NULL ; | NULL |
| REMARK | 3 | SPHERICITY; FREE ATOMS | (A**2): | NULL ; | NULL ; | NULL |
| REMARK | 3 | SPHERICITY; BONDED ATOMS | (A**2): | NULL ; | NULL ; | NULL |
| REMARK | 3 | | | | | |

| REMARK | 3 | NCS RESTRAINTS STATISTICS | | |
|---|---|---|---|---|
| REMARK | 3 | NUMBER OF DIFFERENT NCS GROUPS : 0 | | |
| REMARK | 3 | | | |
| REMARK | 3 | TLS DETAILS | | |
| REMARK | 3 | NUMBER OF TLS GROUPS : 5 | | |
| REMARK | 3 | | | |
| REMARK | 3 | TLS GROUP : 1 | | |
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | A 1 | A 333 |
| REMARK | 3 | ORIGIN FOR THE GROUP (A): −54.3940 −41.4280 76.3710 | | |
| REMARK | 3 | T TENSOR | | |
| REMARK | 3 | T11: 0.0488 T22: −0.4021 | | |
| REMARK | 3 | T33: −0.3291 T12: 0.2088 | | |
| REMARK | 3 | T13: 0.4594 T23: 0.1594 | | |
| REMARK | 3 | L TENSOR | | |
| REMARK | 3 | L11: 3.2010 L22: 4.9182 | | |
| REMARK | 3 | L33: 2.6407 L12: 0.0206 | | |
| REMARK | 3 | L13: −1.1756 L23: −1.5965 | | |
| REMARK | 3 | S TENSOR | | |
| REMARK | 3 | S11: 0.1180 S12: −0.1105 S13: 0.3216 | | |
| REMARK | 3 | S21: 0.4136 S22: 0.2256 S23: 0.6029 | | |
| REMARK | 3 | S31: −0.4494 S32: −0.5301 S33: −0.3437 | | |
| REMARK | 3 | | | |
| REMARK | 3 | TLS GROUP : 2 | | |
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | A 377 | A 713 |
| REMARK | 3 | ORIGIN FOR THE GROUP (A): −32.7180 −65.7300 95.6120 | | |
| REMARK | 3 | T TENSOR | | |
| REMARK | 3 | T11: −0.2332 T22: −0.5344 | | |
| REMARK | 3 | T33: −0.4849 T12: 0.1358 | | |
| REMARK | 3 | T13: 0.0423 T23: 0.0906 | | |
| REMARK | 3 | L TENSOR | | |
| REMARK | 3 | L11: 5.2257 L22: 4.3633 | | |
| REMARK | 3 | L33: 3.6873 L12: 1.8858 | | |
| REMARK | 3 | L13: −0.9924 L23: −1.8479 | | |
| REMARK | 3 | S TENSOR | | |
| REMARK | 3 | S11: 0.0737 S12: −0.3120 S13: −0.1871 | | |
| REMARK | 3 | S21: 0.4170 S22: −0.1297 S23: −0.5661 | | |
| REMARK | 3 | S31: −0.3564 S32: 0.4305 S33: 0.0559 | | |
| REMARK | 3 | | | |
| REMARK | 3 | TLS GROUP : 3 | | |
| REMARK | 3 | NUMBER OF COMPONENT GROUP : 1 | | |
| REMARK | 3 | COMPONENTS | C SSSEQI TO | C SSSEQI |
| REMARK | 3 | RESIDUE RANGE : | B 1691 | B 2017 |
| REMARK | 3 | ORIGIN FOR THE GROUP (A): −20.3410 −40.3280 73.1920 | | |
| REMARK | 3 | T TENSOR | | |
| REMARK | 3 | T11: 0.0369 T22: −0.4212 | | |
| REMARK | 3 | T33: −0.3314 T12: −0.1045 | | |
| REMARK | 3 | T13: 0.1959 T23: 0.0659 | | |
| REMARK | 3 | L TENSOR | | |
| REMARK | 3 | L11: 4.9932 L22: 2.3390 | | |
| REMARK | 3 | L33: 3.6103 L12: −0.5468 | | |
| REMARK | 3 | L13: 0.2394 L23: −0.1885 | | |
| REMARK | 3 | S TENSOR | | |
| REMARK | 3 | S11: −0.0733 S12: −0.3626 S13: 0.1996 | | |
| REMARK | 3 | S21: 0.6314 S22: 0.0715 S23: −0.6262 | | |
| REMARK | 3 | S31: −0.5013 S32: 0.6131 S33: 0.0017 | | |
| REMARK | 3 | | | |
| REMARK | 3 | TLS GROUP : 4 | | |
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | B 2018 | B 2172 |
| REMARK | 3 | ORIGIN FOR THE GROUP (A): −15.6000 −17.4400 45.2720 | | |
| REMARK | 3 | T TENSOR | | |
| REMARK | 3 | T11: −0.2260 T22: −0.4864 | | |
| REMARK | 3 | T33: −0.6626 T12: −0.1539 | | |
| REMARK | 3 | T13: 0.2109 T23: −0.0963 | | |
| REMARK | 3 | L TENSOR | | |
| REMARK | 3 | L11: 9.1841 L22: 6.5501 | | |
| REMARK | 3 | L33: 8.5469 L12: 3.1423 | | |
| REMARK | 3 | L13: −4.7271 L23: −2.7307 | | |
| REMARK | 3 | S TENSOR | | |
| REMARK | 3 | S11: 0.2568 S12: −0.1740 S13: 0.6834 | | |
| REMARK | 3 | S21: 0.4176 S22: 0.0791 S23: 0.0414 | | |
| REMARK | 3 | S31: −0.8275 S32: 0.9388 S33: −0.3360 | | |
| REMARK | 3 | | | |
| REMARK | 3 | TLS GROUP : 5 | | |
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | B 2173 | B 2332 |

| REMARK | 3 | ORIGIN FOR THE GROUP (A): −47.5410 −12.1740 37.1510 | | |
|---|---|---|---|---|
| REMARK | 3 | T TENSOR | | |
| REMARK | 3 | T11: −0.2882 T22: −0.6015 | | |
| REMARK | 3 | T33: −0.2609 T12: −0.0841 | | |
| REMARK | 3 | T13: 0.2885 T23: 0.0570 | | |
| REMARK | 3 | L TENSOR | | |
| REMARK | 3 | L11: 6.9284 L22: 7.8931 | | |
| REMARK | 3 | L33: 8.5099 L12: −0.1438 | | |
| REMARK | 3 | L13: −1.5144 L23: −4.0847 | | |
| REMARK | 3 | S TENSOR | | |
| REMARK | 3 | S11: 0.0258 S12: −0.1522 S13: −0.2533 | | |
| REMARK | 3 | S21: 0.2658 S22: 0.2510 S23: 0.8421 | | |
| REMARK | 3 | S31: 0.2954 S32: −0.6847 S33: −0.2767 | | |
| REMARK | 3 | | | |
| REMARK | 3 | BULK SOLVENT MODELLING. | | |
| REMARK | 3 | METHOD USED : MASK | | |
| REMARK | 3 | PARAMETERS FOR MASK CALCULATION | | |
| REMARK | 3 | VDW PROBE RADIUS : 1.20 | | |
| REMARK | 3 | ION PROBE RADIUS : 0.80 | | |
| REMARK | 3 | SHRINKAGE RADIUS : 0.80 | | |
| REMARK | 3 | | | |
| REMARK | 3 | OTHER REFINEMENT REMARKS: HYDROGENS HAVE BEEN ADDED IN THE | | |
| REMARK | 3 | RIDING POSITIONS | | |
| REMARK | 4 | | | |
| REMARK | 4 | 3CDZ COMPLIES WITH FORMAT V. 3.1, 01-AUG-2007 | | |
| REMARK | 100 | | | |
| REMARK | 100 | THIS ENTRY HAS BEEN PROCESSED BY RCSB. | | |
| REMARK | 100 | THE RCSB ID CODE IS RCSB046659. | | |
| REMARK | 200 | | | |
| REMARK | 200 | EXPERIMENTAL DETAILS | | |
| REMARK | 200 | EXPERIMENT TYPE | : | X-RAY DIFFRACTION |
| REMARK | 200 | DATE OF DATA COLLECTION | : | NULL |
| REMARK | 200 | TEMPERATURE | (KELVIN) : | NULL |
| REMARK | 200 | PH | : | 8.50 |
| REMARK | 200 | NUMBER OF CRYSTALS USED | : | 1 |
| REMARK | 200 | | | |
| REMARK | 200 | SYNCHROTRON | (Y/N) : | Y |
| REMARK | 200 | RADIATION SOURCE | : | APS |
| REMARK | 200 | BEAMLINE | : | 24-ID-C |
| REMARK | 200 | X-RAY GENERATOR MODEL | : | NULL |
| REMARK | 200 | MONOCHROMATIC OR LAUE | (M/L) : | M |
| REMARK | 200 | WAVELENGTH OR RANGE | (A) : | 0.97918 |
| REMARK | 200 | MONOCHROMATOR | : | NULL |
| REMARK | 200 | OPTICS | : | NULL |
| REMARK | 200 | | | |
| REMARK | 200 | DETECTOR TYPE | : | CCD |
| REMARK | 200 | DETECTOR MANUFACTURER | : | ADSC QUANTUM 315 |
| REMARK | 200 | INTENSITY-INTEGRATION SOFTWARE | : | HKL-2000 |
| REMARK | 200 | DATA SCALING SOFTWARE | : | HKL-2000 |
| REMARK | 200 | | | |
| REMARK | 200 | NUMBER OF UNIQUE REFLECTIONS | : | 26444 |
| REMARK | 200 | RESOLUTION RANGE HIGH | (A) : | 3.980 |
| REMARK | 200 | RESOLUTION RANGE LOW | (A) : | 50.000 |
| REMARK | 200 | REJECTION CRITERIA | (SIGMA(I)) : | 1.000 |
| REMARK | 200 | | | |
| REMARK | 200 | OVERALL. | | |
| REMARK | 200 | COMPLETENESS FOR RANGE | (%) : | 99.0 |
| REMARK | 200 | DATA REDUNDANCY | : | 13.200 |
| REMARK | 200 | R MERGE | (I) : | 0.13000 |
| REMARK | 200 | R SYM | (I) : | NULL |
| REMARK | 200 | <I/SIGMA(I)> FOR THE DATA SET | : | 27.2000 |
| REMARK | 200 | | | |

| REMARK | 200 | IN THE HIGHEST RESOLUTION SHELL. | | |
|---|---|---|---|---|
| REMARK | 200 | HIGHEST RESOLUTION SHELL, RANGE HIGH | (A) : | NULL |
| REMARK | 200 | HIGHEST RESOLUTION SHELL, RANGE LOW | (A) : | NULL |

| | | | | |
|---|---|---|---|---|
| REMARK | 200 | COMPLETENESS FOR SHELL | (%) : | 92.3 |
| REMARK | 200 | DATA REDUNDANCY IN SHELL | : | 8.90 |
| REMARK | 200 | R MERGE FOR SHELL | (I) : | 0.82700 |
| REMARK | 200 | R SYM FOR SHELL | (I) : | NULL |
| REMARK | 200 | <I/SIGMA(I)> FOR SHELL | : | 1.700 |
| REMARK | 200 | | | |

| REMARK | 200 | DIFFRACTION PROTOCOL: SINGLE WAVELENGTH | |
|---|---|---|---|
| REMARK | 200 | METHOD USED TO DETERMINE THE STRUCTURE: MOLECULAR REPLACEMENT | |
| REMARK | 200 | SOFTWARE USED: AMORE | |
| REMARK | 200 | STARTING MODEL: HOMOLOGY MODELS OF INDIVIDUAL DOMAIN BUILT BY | |
| REMARK | 200 | SWISS-MODEL USING PDB ENTRIES 1SDD, 1KCW AND 1D7P. | |
| REMARK | 200 | | |
| REMARK | 200 | REMARK: NULL | |
| REMARK | 280 | | |
| REMARK | 280 | CRYSTAL | |
| REMARK | 280 | SOLVENT CONTENT, VS (%): 74.11 | |
| REMARK | 280 | MATTHEWS COEFFICIENT, VM (ANGSTROMS**3/DA): 4.75 | |
| REMARK | 280 | | |
| REMARK | 280 | CRYSTALLIZATION CONDITIONS: 100 MM TRIS-HCL, 10% ETOH, 7% PEG | |
| REMARK | 280 | 3350, PH 8.5, VAPOR DIFFUSION, HANGING DROP, TEMPERATURE 298 K | |
| REMARK | 290 | | |
| REMARK | 290 | CRYSTALLOGRAPHIC SYMMETRY | |
| REMARK | 290 | SYMMETRY OPERATORS FOR SPACE GROUP: P 41 21 2 | |
| REMARK | 290 | | |
| REMARK | 290 | SYMOP | SYMMETRY |
| REMARK | 290 | NNNMMM | OPERATOR |
| REMARK | 290 | 1555 | X, Y, Z |
| REMARK | 290 | 2555 | −X, −Y, 1/2 + Z |
| REMARK | 290 | 3555 | 1/2 − Y, 1/2 + X, 1/4 + Z |
| REMARK | 290 | 4555 | 1/2 + Y, 1/2 − X, 3/4 + Z |
| REMARK | 290 | 5555 | 1/2 − X, 1/2 + Y, 1/4 − Z |
| REMARK | 290 | 6555 | 1/2 + X, 1/2 − Y, 3/4 − Z |
| REMARK | 290 | 7555 | Y, X, −Z |
| REMARK | 290 | 8555 | −Y, −X, 1/2 − Z |
| REMARK | 290 | | |

| REMARK | 290 | WHERE | NNN -> OPERATOR NUMBER | | | | | |
|---|---|---|---|---|---|---|---|---|
| REMARK | 290 | | MMM -> TRANSLATION VECTOR | | | | | |
| REMARK | 290 | | | | | | | |
| REMARK | 290 | CRYSTALLOGRAPHIC SYMMETRY TRANSFORMATIONS | | | | | | |
| REMARK | 290 | THE FOLLOWING TRANSFORMATIONS OPERATE ON THE ATOM/HETATM | | | | | | |
| REMARK | 290 | RECORDS IN THIS ENTRY TO PRODUCE CRYSTALLOGRAPHICALLY | | | | | | |
| REMARK | 290 | RELATED MOLECULES. | | | | | | |
| REMARK | 290 | SMTRY1 | 1 | 1.000000 | 0.000000 | 0.000000 | 0.00000 | |
| REMARK | 290 | SMTRY2 | 1 | 0.000000 | 1.000000 | 0.000000 | 0.00000 | |
| REMARK | 290 | SMTRY3 | 1 | 0.000000 | 0.000000 | 1.000000 | 0.00000 | |
| REMARK | 290 | SMTRY1 | 2 | −1.000000 | 0.000000 | 0.000000 | 0.00000 | |
| REMARK | 290 | SMTRY2 | 2 | 0.000000 | −1.000000 | 0.000000 | 0.00000 | |
| REMARK | 290 | SMTRY3 | 2 | 0.000000 | 0.000000 | 1.000000 | 174.88000 | |
| REMARK | 290 | SMTRY1 | 3 | 0.000000 | −1.000000 | 0.000000 | 67.05650 | |
| REMARK | 290 | SMTRY2 | 3 | 1.000000 | 0.000000 | 0.000000 | 67.05650 | |
| REMARK | 290 | SMTRY3 | 3 | 0.000000 | 0.000000 | 1.000000 | 87.44000 | |
| REMARK | 290 | SMTRY1 | 4 | 0.000000 | 1.000000 | 0.000000 | 67.05650 | |
| REMARK | 290 | SMTRY2 | 4 | −1.000000 | 0.000000 | 0.000000 | 67.05650 | |
| REMARK | 290 | SMTRY3 | 4 | 0.000000 | 0.000000 | 1.000000 | 262.32000 | |
| REMARK | 290 | SMTRY1 | 5 | −1.000000 | 0.000000 | 0.000000 | 67.05650 | |
| REMARK | 290 | SMTRY2 | 5 | 0.000000 | 1.000000 | 0.000000 | 67.05650 | |
| REMARK | 290 | SMTRY3 | 5 | 0.000000 | 0.000000 | −1.000000 | 87.44000 | |
| REMARK | 290 | SMTRY1 | 6 | 1.000000 | 0.000000 | 0.000000 | 67.05650 | |
| REMARK | 290 | SMTRY2 | 6 | 0.000000 | −1.000000 | 0.000000 | 67.05650 | |
| REMARK | 290 | SMTRY3 | 6 | 0.000000 | 0.000000 | −1.000000 | 262.32000 | |
| REMARK | 290 | SMTRY1 | 7 | 0.000000 | 1.000000 | 0.000000 | 0.00000 | |
| REMARK | 290 | SMTRY2 | 7 | 1.000000 | 0.000000 | 0.000000 | 0.00000 | |
| REMARK | 290 | SMTRY3 | 7 | 0.000000 | 0.000000 | −1.000000 | 0.00000 | |
| REMARK | 290 | SMTRY1 | 8 | 0.000000 | −1.000000 | 0.000000 | 0.00000 | |
| REMARK | 290 | SMTRY2 | 8 | −1.000000 | 0.000000 | 0.000000 | 0.00000 | |
| REMARK | 290 | SMTRY3 | 8 | 0.000000 | 0.000000 | −1.000000 | 174.88000 | |
| REMARK | 290 | | | | | | | |
| REMARK | 290 | REMARK: NULL | | | | | | |
| REMARK | 300 | | | | | | | |
| REMARK | 300 | BIOMOLECULE: 1 | | | | | | |
| REMARK | 300 | SEE REMARK 350 FOR THE AUTHOR PROVIDED AND/OR PROGRAM | | | | | | |
| REMARK | 300 | GENERATED ASSEMBLY INFORMATION FOR THE STRUCTURE IN | | | | | | |
| REMARK | 300 | THIS ENTRY. THE REMARK MAY ALSO PROVIDE INFORMATION ON | | | | | | |
| REMARK | 300 | BURIED SURFACE AREA. | | | | | | |
| REMARK | 350 | | | | | | | |
| REMARK | 350 | COORDINATES FOR A COMPLETE MULTIMER REPRESENTING THE KNOWN | | | | | | |
| REMARK | 350 | BIOLOGICALLY SIGNIFICANT OLIGOMERIZATION STATE OF THE | | | | | | |
| REMARK | 350 | MOLECULE CAN BE GENERATED BY APPLYING BIOMT TRANSFORMATIONS | | | | | | |
| REMARK | 350 | GIVEN BELOW. BOTH NON-CRYSTALLOGRAPHIC AND | | | | | | |
| REMARK | 350 | CRYSTALLOGRAPHIC OPERATIONS ARE GIVEN. | | | | | | |
| REMARK | 350 | | | | | | | |
| REMARK | 350 | BIOMOLECULE: 1 | | | | | | |
| REMARK | 350 | AUTHOR DETERMINED BIOLOGICAL UNIT: DIMERIC | | | | | | |
| REMARK | 350 | SOFTWARE DETERMINED QUATERNARY STRUCTURE: DIMERIC | | | | | | |
| REMARK | 350 | SOFTWARE USED: PISA | | | | | | |
| REMARK | 350 | TOTAL BURIED SURFACE AREA: 9180 ANGSTROM**2 | | | | | | |
| REMARK | 350 | TOTAL SURFACE AREA FOR THE COMPLEX: 56090 ANGSTROM**2 | | | | | | |
| REMARK | 350 | GAIN IN SOLVENT FREE ENERGY: −23 KCAL/MOL | | | | | | |
| REMARK | 350 | APPLY THE FOLLOWING TO CHAINS: A, B | | | | | | |
| REMARK | 350 | BIOMT1 | 1 | 1.000000 | 0.000000 | 0.000000 | | 0.00000 |
| REMARK | 350 | BIOMT2 | 1 | 0.000000 | 1.000000 | 0.000000 | | 0.00000 |
| REMARK | 350 | BIOMT3 | 1 | 0.000000 | 0.000000 | 1.000000 | | 0.00000 |
| REMARK | 465 | | | | | | | |

| REMARK | 465 | MISSING RESIDUES | | | |
|---|---|---|---|---|---|
| REMARK | 465 | THE FOLLOWING RESIDUES WERE NOT LOCATED IN THE | | | |
| REMARK | 465 | EXPERIMENT. (M = MODEL NUMBER; RES = RESIDUE NAME; C = CHAIN | | | |
| REMARK | 465 | IDENTIFIER; SSEQ = SEQUENCE NUMBER; I = INSERTION CODE.) | | | |
| REMARK | 465 | | | | |
| REMARK | 465 | M | RES | C | SSEQI |
| REMARK | 465 | | MET | A | 17 |
| REMARK | 465 | | GLN | A | 18 |
| REMARK | 465 | | SER | A | 19 |
| REMARK | 465 | | ASP | A | 20 |
| REMARK | 465 | | LEU | A | 21 |
| REMARK | 465 | | GLY | A | 22 |
| REMARK | 465 | | GLU | A | 23 |
| REMARK | 465 | | LEU | A | 24 |
| REMARK | 465 | | PRO | A | 25 |
| REMARK | 465 | | VAL | A | 26 |
| REMARK | 465 | | ASP | A | 27 |
| REMARK | 465 | | ALA | A | 28 |
| REMARK | 465 | | ARG | A | 29 |
| REMARK | 465 | | PHE | A | 30 |
| REMARK | 465 | | PRO | A | 31 |
| REMARK | 465 | | PRO | A | 32 |
| REMARK | 465 | | ARG | A | 33 |
| REMARK | 465 | | VAL | A | 34 |
| REMARK | 465 | | PRO | A | 35 |
| REMARK | 465 | | LYS | A | 36 |
| REMARK | 465 | | SER | A | 37 |
| REMARK | 465 | | PHE | A | 38 |
| REMARK | 465 | | PRO | A | 39 |
| REMARK | 465 | | PHE | A | 40 |
| REMARK | 465 | | ASN | A | 41 |
| REMARK | 465 | | THR | A | 42 |
| REMARK | 465 | | SER | A | 43 |
| REMARK | 465 | | GLU | A | 211 |
| REMARK | 465 | | THR | A | 212 |
| REMARK | 465 | | LYS | A | 213 |
| REMARK | 465 | | ASN | A | 214 |
| REMARK | 465 | | SER | A | 215 |
| REMARK | 465 | | LEU | A | 216 |
| REMARK | 465 | | MET | A | 217 |
| REMARK | 465 | | GLN | A | 218 |
| REMARK | 465 | | ASP | A | 219 |
| REMARK | 465 | | ARG | A | 220 |
| REMARK | 465 | | ASP | A | 221 |
| REMARK | 465 | | ALA | A | 222 |
| REMARK | 465 | | ALA | A | 223 |
| REMARK | 465 | | GLN | A | 334 |
| REMARK | 465 | | LEU | A | 335 |
| REMARK | 465 | | ARG | A | 336 |
| REMARK | 465 | | MET | A | 337 |
| REMARK | 465 | | LYS | A | 338 |
| REMARK | 465 | | ASN | A | 339 |
| REMARK | 465 | | ASN | A | 340 |
| REMARK | 465 | | GLU | A | 341 |
| REMARK | 465 | | GLU | A | 342 |
| REMARK | 465 | | ALA | A | 343 |
| REMARK | 465 | | GLU | A | 344 |
| REMARK | 465 | | ASP | A | 345 |
| REMARK | 465 | | TYR | A | 346 |
| REMARK | 465 | | ASP | A | 347 |
| REMARK | 465 | | ASP | A | 348 |
| REMARK | 465 | | ASP | A | 349 |
| REMARK | 465 | | LEU | A | 350 |
| REMARK | 465 | | THR | A | 351 |
| REMARK | 465 | | ASP | A | 352 |
| REMARK | 465 | | SER | A | 353 |
| REMARK | 465 | | GLU | A | 354 |
| REMARK | 465 | | MET | A | 355 |
| REMARK | 465 | | ASP | A | 356 |
| REMARK | 465 | | VAL | A | 357 |
| REMARK | 465 | | VAL | A | 358 |
| REMARK | 465 | | ARG | A | 359 |
| REMARK | 465 | | PHE | A | 360 |
| REMARK | 465 | | ASP | A | 361 |
| REMARK | 465 | | ASP | A | 362 |
| REMARK | 465 | | ASP | A | 363 |
| REMARK | 465 | | ASN | A | 364 |
| REMARK | 465 | | SER | A | 365 |
| REMARK | 465 | | PRO | A | 366 |
| REMARK | 465 | | SER | A | 367 |
| REMARK | 465 | | PHE | A | 368 |
| REMARK | 465 | | ILE | A | 369 |
| REMARK | 465 | | GLN | A | 370 |
| REMARK | 465 | | ILE | A | 371 |
| REMARK | 465 | | ARG | A | 372 |
| REMARK | 465 | | SER | A | 373 |
| REMARK | 465 | | VAL | A | 374 |
| REMARK | 465 | | ALA | A | 375 |
| REMARK | 465 | | LYS | A | 376 |
| REMARK | 465 | | ASN | A | 714 |
| REMARK | 465 | | THR | A | 715 |
| REMARK | 465 | | GLY | A | 716 |
| REMARK | 465 | | ASP | A | 717 |
| REMARK | 465 | | TYR | A | 718 |
| REMARK | 465 | | TYR | A | 719 |
| REMARK | 465 | | GLU | A | 720 |
| REMARK | 465 | | ASP | A | 721 |
| REMARK | 465 | | SER | A | 722 |
| REMARK | 465 | | TYR | A | 723 |
| REMARK | 465 | | GLU | A | 724 |
| REMARK | 465 | | ASP | A | 725 |
| REMARK | 465 | | ILE | A | 726 |
| REMARK | 465 | | SER | A | 727 |
| REMARK | 465 | | ALA | A | 728 |
| REMARK | 465 | | TYR | A | 729 |
| REMARK | 465 | | LEU | A | 730 |
| REMARK | 465 | | LEU | A | 731 |
| REMARK | 465 | | SER | A | 732 |
| REMARK | 465 | | LYS | A | 733 |
| REMARK | 465 | | ASN | A | 734 |
| REMARK | 465 | | ASN | A | 735 |
| REMARK | 465 | | ALA | A | 736 |
| REMARK | 465 | | ILE | A | 737 |
| REMARK | 465 | | GLU | A | 738 |
| REMARK | 465 | | PRO | A | 739 |
| REMARK | 465 | | ARG | A | 740 |
| REMARK | 465 | | SER | A | 741 |
| REMARK | 465 | | PHE | A | 742 |
| REMARK | 465 | | SER | A | 743 |
| REMARK | 465 | | GLN | A | 744 |
| REMARK | 465 | | ASN | A | 745 |
| REMARK | 465 | | PRO | A | 746 |
| REMARK | 465 | | PRO | A | 747 |
| REMARK | 465 | | VAL | A | 748 |
| REMARK | 465 | | LEU | A | 749 |
| REMARK | 465 | | LYS | A | 750 |
| REMARK | 465 | | ARG | A | 751 |
| REMARK | 465 | | HIS | A | 752 |
| REMARK | 465 | | GLN | A | 753 |
| REMARK | 465 | | ARG | A | 754 |
| REMARK | 465 | | GLU | B | 1649 |
| REMARK | 465 | | ILE | B | 1650 |
| REMARK | 465 | | THR | B | 1651 |
| REMARK | 465 | | ARG | B | 1652 |
| REMARK | 465 | | THR | B | 1653 |
| REMARK | 465 | | THR | B | 1654 |
| REMARK | 465 | | LEU | B | 1655 |
| REMARK | 465 | | GLN | B | 1656 |
| REMARK | 465 | | SER | B | 1657 |
| REMARK | 465 | | ASP | B | 1658 |
| REMARK | 465 | | GLN | B | 1659 |
| REMARK | 465 | | GLU | B | 1660 |
| REMARK | 465 | | GLU | B | 1661 |
| REMARK | 465 | | ILE | B | 1662 |
| REMARK | 465 | | ASP | B | 1663 |
| REMARK | 465 | | TYR | B | 1664 |
| REMARK | 465 | | ASP | B | 1665 |
| REMARK | 465 | | ASP | B | 1666 |
| REMARK | 465 | | THR | B | 1667 |
| REMARK | 465 | | ILE | B | 1668 |
| REMARK | 465 | | SER | B | 1669 |
| REMARK | 465 | | VAL | B | 1670 |
| REMARK | 465 | | GLU | B | 1671 |
| REMARK | 465 | | MET | B | 1672 |
| REMARK | 465 | | LYS | B | 1673 |
| REMARK | 465 | | LYS | B | 1674 |
| REMARK | 465 | | GLU | B | 1675 |
| REMARK | 465 | | ASP | B | 1676 |
| REMARK | 465 | | PHE | B | 1677 |
| REMARK | 465 | | ASP | B | 1678 |
| REMARK | 465 | | ILE | B | 1679 |
| REMARK | 465 | | TYR | B | 1680 |
| REMARK | 465 | | ASP | B | 1681 |
| REMARK | 465 | | GLU | B | 1682 |
| REMARK | 465 | | ASP | B | 1683 |
| REMARK | 465 | | GLU | B | 1684 |
| REMARK | 465 | | ASN | B | 1685 |
| REMARK | 465 | | GLN | B | 1686 |
| REMARK | 465 | | SER | B | 1687 |
| REMARK | 465 | | PRO | B | 1688 |
| REMARK | 465 | | ARG | B | 1689 |
| REMARK | 465 | | SER | B | 1690 |
| REMARK | 465 | | SER | B | 1714 |
| REMARK | 465 | | PRO | B | 1715 |
| REMARK | 465 | | HIS | B | 1716 |
| REMARK | 465 | | VAL | B | 1717 |
| REMARK | 465 | | LEU | B | 1718 |
| REMARK | 465 | | ARG | B | 1719 |
| REMARK | 465 | | ASN | B | 1720 |
| REMARK | 465 | | ARG | B | 1721 |
| REMARK | 465 | | ALA | B | 1722 |
| REMARK | 465 | | GLN | B | 1723 |
| REMARK | 465 | | SER | B | 1724 |
| REMARK | 500 | | | | |

| REMARK | 500 | GEOMETRY AND STEREOCHEMISTRY | | | | | | | | |
|---|---|---|---|---|---|---|---|---|---|---|
| REMARK | 500 | SUBTOPIC: CLOSE CONTACTS IN SAME ASYMMETRIC UNIT | | | | | | | | |
| REMARK | 500 | | | | | | | | | |
| REMARK | 500 | THE FOLLOWING ATOMS ARE IN CLOSE CONTACT. | | | | | | | | |
| REMARK | 500 | | | | | | | | | |
| REMARK | 500 | ATM1 | RES | C | SSEQI | ATM2 | RES | C | SSEQI | |
| REMARK | 500 | CG | ASN | B | 2118 | C1 | NAG | B | 2334 | 2.06 |
| REMARK | 500 | OH | TYR | A | 114 | NH1 | ARG | B | 1997 | 2.08 |
| REMARK | 500 | O | GLY | A | 643 | NE2 | GLN | A | 645 | 2.09 |
| REMARK | 500 | O | PRO | A | 492 | N | GLY | A | 494 | 2.18 |
| REMARK | 500 | | | | | | | | | |

| REMARK | 500 | REMARK: NULL | | | | | | | |
|---|---|---|---|---|---|---|---|---|---|
| REMARK | 500 | | | | | | | | |
| REMARK | 500 | GEOMETRY AND STEREOCHEMISTRY | | | | | | | |
| REMARK | 500 | SUBTOPIC: COVALENT BOND LENGTHS | | | | | | | |
| REMARK | 500 | | | | | | | | |
| REMARK | 500 | THE STEREOCHEMICAL PARAMETERS OF THE FOLLOWING RESIDUES | | | | | | | |
| REMARK | 500 | HAVE VALUES WHICH DEVIATE FROM EXPECTED VALUES BY MORE | | | | | | | |
| REMARK | 500 | THAN 6*RMSD (M = MODEL NUMBER; RES = RESIDUE NAME; C = CHAIN | | | | | | | |
| REMARK | 500 | IDENTIFIER; SSEQ = SEQUENCE NUMBER; I = INSERTION CODE). | | | | | | | |
| REMARK | 500 | | | | | | | | |
| REMARK | 500 | STANDARD TABLE: | | | | | | | |
| REMARK | 500 | FORMAT: (10X, I3, 1X, 2(A3, 1X, A1, I4, A1, 1X, A4, 3X), F6.3) | | | | | | | |
| REMARK | 500 | | | | | | | | |
| REMARK | 500 | EXPECTED VALUES PROTEIN: ENGH AND HUBER, 1999 | | | | | | | |
| REMARK | 500 | EXPECTED VALUES NUCELIC ACID: CLOWNEY ET AL 1996 | | | | | | | |
| REMARK | 500 | | | | | | | | |
| REMARK | 500 | M | RES | CSSEQI | ATM1 | RES | CSSEQI | ATM2 | DEVIATION |
| REMARK | 500 | | LYS | A 107 | CG | LYS | A 107 | CD | 0.222 |
| REMARK | 500 | | ARG | A 121 | NE | ARG | A 121 | CZ | 0.113 |
| REMARK | 500 | | ARG | A 121 | CZ | ARG | A 121 | NH1 | 0.171 |
| REMARK | 500 | | ARG | A 121 | CZ | ARG | A 121 | NH2 | 0.084 |
| REMARK | 500 | | GLN | A 602 | CD | GLN | A 602 | NE2 | 0.159 |
| REMARK | 500 | | ASN | B1904 | CG | ASN | B1904 | OD1 | 0.214 |
| REMARK | 500 | | ASN | B1904 | CG | ASN | B1904 | ND2 | 0.208 |
| REMARK | 500 | | | | | | | | |

| REMARK | 500 | REMARK: NULL | | | | | | | | |
|---|---|---|---|---|---|---|---|---|---|---|
| REMARK | 500 | | | | | | | | | |
| REMARK | 500 | GEOMETRY AND STEREOCHEMISTRY | | | | | | | | |
| REMARK | 500 | SUBTOPIC: COVALENT BOND ANGLES | | | | | | | | |
| REMARK | 500 | | | | | | | | | |
| REMARK | 500 | THE STEREOCHEMICAL PARAMETERS OF THE FOLLOWING RESIDUES | | | | | | | | |
| REMARK | 500 | HAVE VALUES WHICH DEVIATE FROM EXPECTED VALUES BY MORE | | | | | | | | |
| REMARK | 500 | THAN 6*RMSD (M = MODEL NUMBER; RES = RESIDUE NAME; C = CHAIN | | | | | | | | |
| REMARK | 500 | IDENTIFIER; SSEQ = SEQUENCE NUMBER; I = INSERTION CODE). | | | | | | | | |
| REMARK | 500 | | | | | | | | | |
| REMARK | 500 | STANDARD TABLE: | | | | | | | | |
| REMARK | 500 | FORMAT: (10X, I3, 1X, A3, 1X, A1, I4, A1, 3(1X, A4, 2X), 12X, F5.1) | | | | | | | | |
| REMARK | 500 | | | | | | | | | |
| REMARK | 500 | EXPECTED VALUES PROTEIN: ENGH AND HUBER, 1999 | | | | | | | | |
| REMARK | 500 | EXPECTED VALUES NUCELIC ACID: CLOWNEY ET AL 1996 | | | | | | | | |
| REMARK | 500 | | | | | | | | | |
| REMARK | 500 | M | RES | CSSEQI | ATM1 | | ATM2 | | ATM3 | |
| REMARK | 500 | | ARG | A 121 | CD | − | NE | − | CZ | ANGL. DEV. = −9.3 DEGREES |
| REMARK | 500 | | ARG | A 121 | NE | − | CZ | − | NH1 | ANGL. DEV. = 11.8 DEGREES |
| REMARK | 500 | | ARG | A 121 | NE | − | CZ | − | NH2 | ANGL. DEV. = −12.0 DEGREES |
| REMARK | 500 | | LEU | A 184 | CA | − | CB | − | CG | ANGL. DEV. = 13.8 DEGREES |
| REMARK | 500 | | LEU | A 277 | CA | − | CB | − | CG | ANGL. DEV. = 17.2 DEGREES |
| REMARK | 500 | | LEU | A 398 | CA | − | CB | − | CG | ANGL. DEV. = 14.1 DEGREES |
| REMARK | 500 | | LEU | B1945 | CA | − | CB | − | CG | ANGL. DEV. = 17.9 DEGREES |
| REMARK | 500 | | LEU | B2050 | CA | − | CB | − | CG | ANGL. DEV. = 15.7 DEGREES |
| REMARK | 500 | | | | | | | | | |

| REMARK | 500 | REMARK: NULL | | | | | |
|---|---|---|---|---|---|---|---|
| REMARK | 500 | | | | | | |
| REMARK | 500 | GEOMETRY AND STEREOCHEMISTRY | | | | | |
| REMARK | 500 | SUBTOPIC: TORSION ANGLES | | | | | |
| REMARK | 500 | | | | | | |
| REMARK | 500 | TORSION ANGLES OUTSIDE THE EXPECTED RAMACHANDRAN REGIONS: | | | | | |
| REMARK | 500 | (M = MODEL NUMBER; RES = RESIDUE NAME; C = CHAIN IDENTIFIER; | | | | | |
| REMARK | 500 | SSEQ = SEQUENCE NUMBER; I = INSERTION CODE). | | | | | |
| REMARK | 500 | | | | | | |
| REMARK | 500 | STANDARD TABLE: | | | | | |
| REMARK | 500 | FORMAT: (10X, I3, 1X, A3, 1X, A1, I4, A1, 4X, F7.2, 3X, F7.2) | | | | | |
| REMARK | 500 | | | | | | |
| REMARK | 500 | EXPECTED VALUES: GJ KLEYWEGT AND TA JONES (1996). PHI/PSI- | | | | | |
| REMARK | 500 | CHOLOGY: RAMACHANDRAN REVISITED. STRUCTURE 4, 1395-1400 | | | | | |
| REMARK | 500 | | | | | | |
| REMARK | 500 | M | RES | CSSEQI | | PSI | PHI |
| REMARK | 500 | | TYR | A | 6 | 44.41 | −74.35 |
| REMARK | 500 | | ASP | A | 15 | −113.61 | −149.85 |
| REMARK | 500 | | TYR | A | 46 | 122.46 | 100.47 |
| REMARK | 500 | | LYS | A | 48 | −158.77 | −115.70 |
| REMARK | 500 | | LEU | A | 50 | −160.23 | −109.23 |
| REMARK | 500 | | VAL | A | 52 | −174.19 | −69.82 |
| REMARK | 500 | | THR | A | 55 | 26.93 | −74.03 |
| REMARK | 500 | | LEU | A | 58 | −110.87 | −116.89 |
| REMARK | 500 | | ILE | A | 61 | 154.26 | −39.80 |
| REMARK | 500 | | ALA | A | 62 | 105.27 | 177.95 |
| REMARK | 500 | | PRO | A | 67 | −123.04 | −91.31 |
| REMARK | 500 | | TRP | A | 68 | −30.57 | −35.94 |
| REMARK | 500 | | THR | A | 75 | 109.01 | −52.87 |
| REMARK | 500 | | VAL | A | 80 | −45.27 | −25.58 |
| REMARK | 500 | | SER | A | 109 | −117.42 | −138.76 |
| REMARK | 500 | | GLU | A | 110 | −57.28 | 71.09 |
| REMARK | 500 | | TYR | A | 114 | 161.93 | 157.56 |
| REMARK | 500 | | ASP | A | 116 | 39.23 | −79.65 |
| REMARK | 500 | | GLN | A | 117 | −57.00 | 65.42 |
| REMARK | 500 | | THR | A | 118 | 114.06 | 29.12 |
| REMARK | 500 | | SER | A | 119 | −174.93 | −62.65 |
| REMARK | 500 | | SER | A | 133 | 125.26 | 142.53 |
| REMARK | 500 | | ALA | A | 148 | −65.44 | −20.06 |
| REMARK | 500 | | ASP | A | 150 | −178.88 | −173.93 |
| REMARK | 500 | | PRO | A | 151 | 116.80 | −21.18 |
| REMARK | 500 | | LEU | A | 168 | −74.65 | −89.16 |
| REMARK | 500 | | GLU | A | 181 | 81.23 | −55.50 |
| REMARK | 500 | | LEU | A | 184 | −20.11 | −38.85 |
| REMARK | 500 | | GLU | A | 204 | −0.62 | −55.50 |
| REMARK | 500 | | SER | A | 207 | −165.64 | −79.46 |
| REMARK | 500 | | ALA | A | 225 | 110.89 | −19.63 |
| REMARK | 500 | | ALA | A | 227 | 72.20 | 168.00 |
| REMARK | 500 | | MET | A | 231 | −174.28 | −173.16 |
| REMARK | 500 | | HIS | A | 232 | 27.41 | −178.60 |
| REMARK | 500 | | VAL | A | 234 | 132.51 | −36.48 |
| REMARK | 500 | | ASN | A | 235 | −7.08 | 54.07 |
| REMARK | 500 | | ASN | A | 239 | −71.58 | 54.54 |
| REMARK | 500 | | ARG | A | 240 | −3.57 | −150.38 |
| REMARK | 500 | | SER | A | 241 | 154.02 | −43.05 |
| REMARK | 500 | | PRO | A | 243 | −92.44 | −85.59 |
| REMARK | 500 | | LYS | A | 251 | −153.68 | −143.13 |
| REMARK | 500 | | PRO | A | 264 | 75.62 | −54.43 |
| REMARK | 500 | | HIS | A | 267 | −169.86 | −121.80 |
| REMARK | 500 | | SER | A | 268 | 104.25 | −169.23 |
| REMARK | 500 | | THR | A | 275 | 44.92 | 80.92 |
| REMARK | 500 | | VAL | A | 278 | −68.36 | −133.32 |
| REMARK | 500 | | ARG | A | 279 | −151.51 | −96.73 |
| REMARK | 500 | | ASN | A | 280 | 34.82 | −78.04 |
| REMARK | 500 | | SER | A | 285 | 117.67 | −175.20 |
| REMARK | 500 | | PRO | A | 290 | −87.65 | −13.95 |
| REMARK | 500 | | LEU | A | 303 | −34.17 | −30.58 |
| REMARK | 500 | | HIS | A | 311 | 56.52 | 37.15 |
| REMARK | 500 | | GLN | A | 316 | −89.55 | −43.40 |
| REMARK | 500 | | HIS | A | 317 | −58.12 | −25.18 |
| REMARK | 500 | | ASP | A | 318 | 45.76 | −95.83 |
| REMARK | 500 | | VAL | A | 324 | 89.22 | −164.74 |
| REMARK | 500 | | VAL | A | 326 | 72.48 | 10.78 |
| REMARK | 500 | | GLU | A | 331 | −1.15 | −57.93 |
| REMARK | 500 | | HIS | A | 378 | −151.12 | −80.64 |
| REMARK | 500 | | PRO | A | 397 | −70.67 | −77.13 |
| REMARK | 500 | | ALA | A | 401 | 44.64 | −159.78 |
| REMARK | 500 | | ASP | A | 403 | −108.85 | −159.34 |
| REMARK | 500 | | LEU | A | 412 | 125.46 | 72.83 |
| REMARK | 500 | | ASN | A | 413 | 77.43 | 38.86 |
| REMARK | 500 | | ARG | A | 418 | 9.57 | −162.28 |
| REMARK | 500 | | ILE | A | 419 | −144.83 | −71.22 |
| REMARK | 500 | | THR | A | 435 | 89.80 | −61.71 |
| REMARK | 500 | | GLN | A | 443 | 74.03 | −113.26 |
| REMARK | 500 | | HIS | A | 444 | 5.59 | −47.66 |
| REMARK | 500 | | SER | A | 470 | −70.38 | −50.72 |
| REMARK | 500 | | ASN | A | 474 | −151.73 | −144.01 |
| REMARK | 500 | | LEU | A | 486 | −70.10 | −36.11 |
| REMARK | 500 | | PRO | A | 492 | 85.30 | −34.99 |
| REMARK | 500 | | LYS | A | 493 | −27.32 | 44.09 |
| REMARK | 500 | | HIS | A | 497 | 140.73 | −178.23 |
| REMARK | 500 | | LEU | A | 498 | −35.39 | −31.85 |
| REMARK | 500 | | LYS | A | 499 | 26.34 | −72.47 |
| REMARK | 500 | | ASP | A | 500 | −74.44 | −94.49 |
| REMARK | 500 | | ILE | A | 503 | 101.86 | 65.59 |
| REMARK | 500 | | PRO | A | 521 | −158.41 | −66.14 |
| REMARK | 500 | | ASP | A | 525 | −157.70 | −54.82 |
| REMARK | 500 | | PRO | A | 526 | −161.88 | −75.88 |
| REMARK | 500 | | ARG | A | 541 | −4.53 | −55.55 |
| REMARK | 500 | | ASP | A | 542 | −81.07 | −107.69 |
| REMARK | 500 | | GLU | A | 557 | 135.16 | 177.67 |
| REMARK | 500 | | SER | A | 558 | 107.44 | −167.89 |
| REMARK | 500 | | VAL | A | 559 | 51.76 | 39.80 |
| REMARK | 500 | | ASP | A | 560 | 166.94 | 170.79 |
| REMARK | 500 | | GLN | A | 561 | −90.26 | 91.84 |
| REMARK | 500 | | ASN | A | 564 | 55.64 | −146.47 |
| REMARK | 500 | | MET | A | 567 | 145.82 | −178.45 |
| REMARK | 500 | | LYS | A | 570 | 170.90 | −45.11 |
| REMARK | 500 | | ASP | A | 580 | 76.81 | −100.51 |
| REMARK | 500 | | ASN | A | 582 | −8.85 | −54.98 |
| REMARK | 500 | | TRP | A | 585 | 15.71 | −63.80 |
| REMARK | 500 | | LEU | A | 587 | −83.70 | −49.54 |
| REMARK | 500 | | ARG | A | 593 | −104.23 | −71.88 |
| REMARK | 500 | | PRO | A | 596 | −84.20 | −57.45 |
| REMARK | 500 | | VAL | A | 601 | 25.98 | −68.03 |
| REMARK | 500 | | LEU | A | 603 | −155.02 | −136.04 |
| REMARK | 500 | | GLU | A | 604 | 97.65 | 19.04 |
| REMARK | 500 | | ILE | A | 613 | 97.72 | −66.95 |
| REMARK | 500 | | LEU | A | 625 | 113.60 | −5.64 |
| REMARK | 500 | | GLN | A | 626 | −123.80 | −74.99 |
| REMARK | 500 | | HIS | A | 632 | −14.71 | 72.17 |
| REMARK | 500 | | GLN | A | 645 | −155.32 | −116.57 |
| REMARK | 500 | | THR | A | 646 | −22.80 | 67.22 |
| REMARK | 500 | | PHE | A | 652 | −111.64 | −87.87 |
| REMARK | 500 | | PHE | A | 653 | 103.49 | 85.80 |
| REMARK | 500 | | LYS | A | 659 | 79.95 | −110.55 |
| REMARK | 500 | | MET | A | 662 | 41.37 | 39.28 |
| REMARK | 500 | | PHE | A | 671 | −138.38 | −66.82 |
| REMARK | 500 | | SER | A | 674 | 98.58 | −57.80 |
| REMARK | 500 | | GLU | A | 683 | 34.47 | −149.63 |
| REMARK | 500 | | SER | A | 695 | 56.61 | −99.85 |
| REMARK | 500 | | ARG | A | 698 | 7.77 | −61.86 |
| REMARK | 500 | | ARG | A | 700 | 23.30 | −78.23 |
| REMARK | 500 | | SER | A | 710 | 154.51 | −49.31 |
| REMARK | 500 | | CYS | A | 711 | −130.63 | −113.76 |
| REMARK | 500 | | ASP | A | 712 | −65.06 | −162.02 |
| REMARK | 500 | | ARG | B | 1705 | −81.97 | −169.78 |
| REMARK | 500 | | LEU | B | 1706 | −103.29 | 132.24 |
| REMARK | 500 | | TYR | B | 1709 | −61.63 | −120.30 |
| REMARK | 500 | | MET | B | 1711 | −71.15 | −131.96 |
| REMARK | 500 | | SER | B | 1712 | −36.49 | −161.98 |
| REMARK | 500 | | VAL | B | 1727 | 136.95 | −35.49 |
| REMARK | 500 | | ASP | B | 1740 | −163.14 | −161.97 |
| REMARK | 500 | | PHE | B | 1743 | 65.50 | 14.84 |
| REMARK | 500 | | TYR | B | 1748 | 123.16 | −18.77 |
| REMARK | 500 | | LEU | B | 1759 | −169.63 | −127.47 |
| REMARK | 500 | | VAL | B | 1767 | −78.94 | −63.79 |
| REMARK | 500 | | ALA | B | 1779 | −173.60 | −176.57 |
| REMARK | 500 | | SER | B | 1780 | −95.79 | −79.89 |
| REMARK | 500 | | TYR | B | 1783 | −167.91 | −121.72 |
| REMARK | 500 | | SER | B | 1788 | −78.85 | −72.07 |
| REMARK | 500 | | LEU | B | 1789 | 91.39 | −62.99 |
| REMARK | 500 | | SER | B | 1791 | 85.18 | −43.17 |
| REMARK | 500 | | TYR | B | 1792 | −179.52 | −68.62 |
| REMARK | 500 | | GLU | B | 1794 | −129.54 | 62.03 |
| REMARK | 500 | | ARG | B | 1797 | 92.48 | −64.41 |
| REMARK | 500 | | GLU | B | 1801 | 66.27 | 67.24 |
| REMARK | 500 | | PRO | B | 1802 | 90.52 | −55.10 |
| REMARK | 500 | | ASN | B | 1810 | −14.60 | 62.51 |
| REMARK | 500 | | GLN | B | 1820 | 170.81 | −59.11 |
| REMARK | 500 | | GLU | B | 1829 | −148.78 | −83.51 |
| REMARK | 500 | | LEU | B | 1843 | −30.59 | −33.76 |
| REMARK | 500 | | ASP | B | 1846 | 18.57 | −67.76 |
| REMARK | 500 | | HIS | B | 1867 | 134.00 | 63.99 |
| REMARK | 500 | | GLN | B | 1870 | −0.76 | −174.96 |
| REMARK | 500 | | VAL | B | 1871 | 84.47 | −54.87 |
| REMARK | 500 | | VAL | B | 1873 | −99.43 | −104.77 |
| REMARK | 500 | | GLN | B | 1874 | 96.10 | 70.43 |
| REMARK | 500 | | GLU | B | 1885 | 66.80 | −111.61 |
| REMARK | 500 | | SER | B | 1888 | 109.57 | 53.35 |
| REMARK | 500 | | TRP | B | 1889 | 14.15 | 25.61 |
| REMARK | 500 | | THR | B | 1892 | 39.45 | −70.67 |
| REMARK | 500 | | GLU | B | 1893 | −42.69 | −147.32 |
| REMARK | 500 | | ASN | B | 1898 | −5.26 | 80.28 |
| REMARK | 500 | | ARG | B | 1900 | −111.10 | −143.70 |
| REMARK | 500 | | CYS | B | 1903 | −150.31 | −130.06 |
| REMARK | 500 | | GLN | B | 1906 | 100.02 | 64.17 |
| REMARK | 500 | | PRO | B | 1910 | 35.89 | −6.17 |
| REMARK | 500 | | PHE | B | 1912 | 82.87 | 13.25 |
| REMARK | 500 | | ARG | B | 1917 | −89.36 | −149.42 |
| REMARK | 500 | | PHE | B | 1918 | 104.45 | −48.82 |
| REMARK | 500 | | ASN | B | 1922 | −49.20 | 143.82 |
| REMARK | 500 | | ILE | B | 1925 | 68.94 | −106.69 |
| REMARK | 500 | | MET | B | 1926 | −120.12 | −70.61 |
| REMARK | 500 | | THR | B | 1928 | 110.07 | 176.29 |
| REMARK | 500 | | PRO | B | 1930 | −87.76 | −73.27 |
| REMARK | 500 | | LEU | B | 1932 | 62.36 | −108.91 |
| REMARK | 500 | | GLN | B | 1936 | 6.63 | −55.62 |
| REMARK | 500 | | ASP | B | 1937 | −73.60 | −124.72 |
| REMARK | 500 | | GLN | B | 1938 | 93.12 | −24.45 |
| REMARK | 500 | | MET | B | 1947 | −135.96 | −133.69 |
| REMARK | 500 | | SER | B | 1949 | −148.34 | −110.62 |
| REMARK | 500 | | ASN | B | 1952 | 34.58 | −75.97 |
| REMARK | 500 | | LYS | B | 1967 | −113.45 | −176.38 |
| REMARK | 500 | | GLU | B | 1969 | −147.79 | −115.45 |
| REMARK | 500 | | ASN | B | 1977 | 87.90 | −58.87 |
| REMARK | 500 | | PRO | B | 1990 | 49.37 | −68.70 |
| REMARK | 500 | | SER | B | 1991 | 18.50 | −61.00 |
| REMARK | 500 | | TRP | B | 1996 | −152.07 | −119.99 |
| REMARK | 500 | | ALA | B | 2008 | 55.46 | −148.56 |
| REMARK | 500 | | SER | B | 2011 | 70.39 | −65.00 |
| REMARK | 500 | | GLN | B | 2022 | 94.78 | −169.34 |
| REMARK | 500 | | SER | B | 2029 | 56.84 | −92.77 |
| REMARK | 500 | | HIS | B | 2031 | 100.67 | −170.03 |
| REMARK | 500 | | ASP | B | 2034 | −91.03 | −65.47 |
| REMARK | 500 | | GLN | B | 2036 | −83.12 | −141.99 |
| REMARK | 500 | | ILE | B | 2037 | −142.41 | −154.47 |
| REMARK | 500 | | THR | B | 2038 | −2.45 | −162.10 |
| REMARK | 500 | | ALA | B | 2039 | −38.05 | −175.16 |
| REMARK | 500 | | GLN | B | 2042 | 135.35 | −172.94 |
| REMARK | 500 | | TYR | B | 2043 | 72.05 | 179.85 |
| REMARK | 500 | | GLN | B | 2045 | 92.25 | −62.49 |
| REMARK | 500 | | TRP | B | 2046 | −166.59 | −75.32 |
| REMARK | 500 | | ALA | B | 2047 | −161.14 | −57.86 |
| REMARK | 500 | | PRO | B | 2048 | 165.48 | −48.98 |
| REMARK | 500 | | LYS | B | 2049 | −36.27 | 122.76 |
| REMARK | 500 | | LEU | B | 2050 | 32.85 | −165.58 |
| REMARK | 500 | | HIS | B | 2054 | −3.13 | 70.71 |
| REMARK | 500 | | TYR | B | 2055 | 106.48 | −56.93 |
| REMARK | 500 | | ILE | B | 2059 | 49.85 | −83.46 |
| REMARK | 500 | | ALA | B | 2061 | −171.50 | 114.12 |
| REMARK | 500 | | SER | B | 2063 | 95.64 | −162.87 |
| REMARK | 500 | | TRP | B | 2070 | 149.72 | 172.81 |
| REMARK | 500 | | ALA | B | 2077 | 143.57 | 174.55 |
| REMARK | 500 | | GLN | B | 2091 | −145.61 | −99.36 |
| REMARK | 500 | | PHE | B | 2093 | 21.88 | −143.25 |
| REMARK | 500 | | ASP | B | 2108 | −34.28 | −145.07 |
| REMARK | 500 | | LYS | B | 2110 | −101.78 | −66.64 |
| REMARK | 500 | | THR | B | 2114 | −168.11 | −64.90 |
| REMARK | 500 | | ASN | B | 2118 | 129.36 | −32.97 |
| REMARK | 500 | | SER | B | 2119 | 31.21 | 33.91 |
| REMARK | 500 | | THR | B | 2120 | −86.07 | −62.99 |
| REMARK | 500 | | ASP | B | 2131 | −155.68 | −155.98 |
| REMARK | 500 | | SER | B | 2133 | 22.39 | −155.85 |
| REMARK | 500 | | ILE | B | 2135 | 81.47 | −65.13 |
| REMARK | 500 | | HIS | B | 2137 | 94.92 | −45.67 |
| REMARK | 500 | | HIS | B | 2155 | 148.73 | −170.67 |
| REMARK | 500 | | SER | B | 2157 | −85.31 | −96.81 |
| REMARK | 500 | | SER | B | 2160 | 101.93 | −53.65 |
| REMARK | 500 | | CYS | B | 2169 | 177.24 | 176.26 |
| REMARK | 500 | | ASN | B | 2172 | 71.94 | −171.15 |
| REMARK | 500 | | CYS | B | 2174 | 62.39 | 30.79 |
| REMARK | 500 | | SER | B | 2194 | 156.43 | 179.38 |
| REMARK | 500 | | ASN | B | 2198 | −92.16 | −143.48 |
| REMARK | 500 | | MET | B | 2199 | −49.20 | −146.75 |
| REMARK | 500 | | SER | B | 2206 | 10.95 | −65.36 |
| REMARK | 500 | | ALA | B | 2208 | 79.32 | −59.17 |
| REMARK | 500 | | LEU | B | 2210 | −36.93 | −28.47 |
| REMARK | 500 | | LYS | B | 2227 | 20.74 | −71.48 |
| REMARK | 500 | | LEU | B | 2251 | −28.19 | 53.18 |
| REMARK | 500 | | LEU | B | 2252 | −89.67 | −62.46 |
| REMARK | 500 | | GLN | B | 2276 | −128.76 | −81.06 |
| REMARK | 500 | | LYS | B | 2279 | −169.68 | −73.30 |
| REMARK | 500 | | ASN | B | 2286 | −178.94 | −64.15 |
| REMARK | 500 | | GLN | B | 2287 | 8.35 | −154.07 |
| REMARK | 500 | | PHE | B | 2290 | −148.25 | −108.08 |
| REMARK | 500 | | THR | B | 2291 | 119.88 | 52.21 |
| REMARK | 500 | | VAL | B | 2294 | 72.55 | −55.56 |
| REMARK | 500 | | ARG | B | 2304 | −18.15 | −143.77 |
| REMARK | 500 | | GLN | B | 2311 | −64.99 | −108.99 |
| REMARK | 500 | | VAL | B | 2314 | −74.17 | −100.00 |
| REMARK | 500 | | CYS | B | 2326 | −135.22 | −157.98 |
| REMARK | 500 | | ALA | B | 2328 | −153.15 | −85.41 |
| REMARK | 500 | | GLN | B | 2329 | 163.70 | −46.57 |
| REMARK | 500 | | LEU | B | 2331 | −46.01 | 73.94 |
| REMARK | 500 | | | | | | |

| REMARK | 500 | REMARK: NULL | | |
|---|---|---|---|---|
| REMARK | 500 | | | |
| REMARK | 500 | GEOMETRY AND STEREOCHEMISTRY | | |
| REMARK | 500 | SUBTOPIC: NON-CIS, NON-TRANS | | |
| REMARK | 500 | | | |
| REMARK | 500 | THE FOLLOWING PEPTIDE BONDS DEVIATE SIGNIFICANTLY FROM BOTH | | |
| REMARK | 500 | CIS AND TRANS CONFORMATION. CIS BONDS, IF ANY, ARE LISTED | | |
| REMARK | 500 | ON CISPEP RECORDS. TRANS IS DEFINED AS 180 +/− 30 AND | | |
| REMARK | 500 | CIS IS DEFINED AS 0 +/− 30 DEGREES. | | |
| REMARK | 500 | | MODEL | OMEGA |

| | | | | | | | | |
|---|---|---|---|---|---|---|---|---|
| REMARK | 500 | ASP | A | 56 | HIS | A | 57 | 138.50 |
| REMARK | 500 | TYR | A | 237 | VAL | A | 238 | −145.68 |
| REMARK | 500 | VAL | A | 278 | ARG | A | 279 | 143.20 |
| REMARK | 500 | HIS | A | 281 | ARG | A | 282 | −147.05 |
| REMARK | 500 | ARG | A | 282 | GLN | A | 283 | −149.49 |
| REMARK | 500 | PHE | A | 293 | LEU | A | 294 | 149.87 |
| REMARK | 500 | PHE | A | 673 | SER | A | 674 | −135.90 |
| REMARK | 500 | ARG | B | 1705 | LEU | B | 1706 | 142.74 |
| REMARK | 500 | LEU | B | 1878 | PHE | B | 1879 | −148.77 |
| REMARK | 500 | GLU | B | 1885 | THR | B | 1886 | 149.38 |
| REMARK | 500 | PRO | B | 2048 | LYS | B | 2049 | −149.46 |
| REMARK | 500 | ASN | B | 2141 | PRO | B | 2142 | 148.96 |
| REMARK | 500 | LEU | B | 2171 | ASN | B | 2172 | 138.42 |
| REMARK | 500 | | | | | | | |

| REMARK | 500 | REMARK: NULL | | | |
|---|---|---|---|---|---|
| REMARK | 620 | | | | |
| REMARK | 620 | METAL COORDINATION | | | |
| REMARK | 620 | (M = MODEL NUMBER; RES = RESIDUE NAME; C = CHAIN IDENTIFIER; | | | |
| REMARK | 620 | SSEQ = SEQUENCE NUMBER; I = INSERTION CODE): | | | |
| REMARK | 620 | | | | |
| REMARK | 620 | COORDINATION ANGLES FOR: M | RES | CSSEQI | METAL |
| REMARK | 620 | | CA | A 758 | CA |

| REMARK | 620 | N | RES | CSSEQI | | ATOM | | | | |
|---|---|---|---|---|---|---|---|---|---|---|
| REMARK | 620 | 1 | LYS | A | 107 | O | | | | |
| REMARK | 620 | 2 | GLU | A | 122 | O | 65.7 | | | |
| REMARK | 620 | 3 | ASP | A | 125 | OD1 | 95.1 | 75.1 | | |
| REMARK | 620 | 4 | ASP | A | 126 | OD1 | 156.8 | 91.1 | 77.3 | |
| REMARK | 620 | 5 | ASP | A | 126 | OD2 | 144.8 | 117.4 | 56.2 | 45.3 |
| REMARK | 620 | N | | | | | 1 | 2 | 3 | 4 |
| REMARK | 620 | | | | | | | | | |

| | | | | | |
|---|---|---|---|---|---|
| REMARK | 620 | COORDINATION ANGLES FOR: M | RES | CSSEQI | METAL |
| REMARK | 620 | | CU | B 1 | CU |

| | | | | | | |
|---|---|---|---|---|---|---|
| REMARK | 620 | N | RES | CSSEQI | ATOM | |
| REMARK | 620 | 1 | HIS | B 1954 | ND1 | |
| REMARK | 620 | 2 | HIS | B 2005 | ND1 | 117.5 |
| REMARK | 620 | N | | | | 1 |
| REMARK | 800 | | | | | |

| REMARK | 800 | SITE | | | | | | | |
|---|---|---|---|---|---|---|---|---|---|
| REMARK | 800 | SITE_IDENTIFIER: AC1 | | | | | | | |
| REMARK | 800 | SITE_DESCRIPTION: NAG BINDING SITE FOR RESIDUE A 755 | | | | | | | |
| REMARK | 800 | SITE_IDENTIFIER: AC2 | | | | | | | |
| REMARK | 800 | SITE_DESCRIPTION: NAG BINDING SITE FOR RESIDUE A 756 | | | | | | | |
| REMARK | 800 | SITE_IDENTIFIER: AC3 | | | | | | | |
| REMARK | 800 | SITE_DESCRIPTION: NAG BINDING SITE FOR RESIDUE B 2333 | | | | | | | |
| REMARK | 800 | SITE_IDENTIFIER: AC4 | | | | | | | |
| REMARK | 800 | SITE_DESCRIPTION: NAG BINDING SITE FOR RESIDUE B 2334 | | | | | | | |
| REMARK | 800 | SITE_IDENTIFIER: AC5 | | | | | | | |
| REMARK | 800 | SITE_DESCRIPTION: NAG BINDING SITE FOR RESIDUE B 2335 | | | | | | | |
| REMARK | 800 | SITE_IDENTIFIER: AC6 | | | | | | | |
| REMARK | 800 | SITE_DESCRIPTION: BMA BINDING SITE FOR RESIDUE B 2336 | | | | | | | |
| REMARK | 800 | SITE_IDENTIFIER: AC7 | | | | | | | |
| REMARK | 800 | SITE_DESCRIPTION: MAN BINDING SITE FOR RESIDUE B 2338 | | | | | | | |
| REMARK | 800 | SITE_IDENTIFIER: AC8 | | | | | | | |
| REMARK | 800 | SITE_DESCRIPTION: CU BINDING SITE FOR RESIDUE B 1 | | | | | | | |
| REMARK | 800 | SITE_IDENTIFIER: AC9 | | | | | | | |
| REMARK | 800 | SITE_DESCRIPTION: CU BINDING SITE FOR RESIDUE A 757 | | | | | | | |
| REMARK | 800 | SITE_IDENTIFIER: BC1 | | | | | | | |
| REMARK | 800 | SITE_DESCRIPTION: CA BINDING SITE FOR RESIDUE A 758 | | | | | | | |
| REMARK | 999 | | | | | | | | |
| REMARK | 999 | SEQUENCE | | | | | | | |
| REMARK | 999 | RESIDUES 741 TO 754 (SFSQNPPVLKRHQR) ARE ENGINEERED LINKER IN | | | | | | | |
| REMARK | 999 | THE CONSTRUCT TO CONNECT THE HEAVY AND LIGHT CHAINS TOGETHER. | | | | | | | |
| REMARK | 999 | INTRACELLULAR CLEAVAGE HAPPEN WITHIN THE LINKER WHEN EXPRESSED, | | | | | | | |
| REMARK | 999 | RESULTING IN TWO SEPARATED HEAVY AND LIGHT CHAINS THAT ASSOCIATE | | | | | | | |
| REMARK | 999 | WITH EACH OTHER. | | | | | | | |
| DBREF | 3CDZ | A | 1 | 740 | UNP | P00451 | FA8_HUMAN | 20 | 759 |
| DBREF | 3CDZ | B | 1649 | 2332 | UNP | P00451 | FA8_HUMAN | 1668 | 2351 |

| SEQADV | 3CDZ | SER | A | 741 | UNP | P00451 | | | SEE REMARK 999 |
|---|---|---|---|---|---|---|---|---|---|
| SEQADV | 3CDZ | PHE | A | 742 | UNP | P00451 | | | SEE REMARK 999 |
| SEQADV | 3CDZ | SER | A | 743 | UNP | P00451 | | | SEE REMARK 999 |
| SEQADV | 3CDZ | GLN | A | 744 | UNP | P00451 | | | SEE REMARK 999 |
| SEQADV | 3CDZ | ASN | A | 745 | UNP | P00451 | | | SEE REMARK 999 |
| SEQADV | 3CDZ | PRO | A | 746 | UNP | P00451 | | | SEE REMARK 999 |
| SEQADV | 3CDZ | PRO | A | 747 | UNP | P00451 | | | SEE REMARK 999 |
| SEQADV | 3CDZ | VAL | A | 748 | UNP | P00451 | | | SEE REMARK 999 |
| SEQADV | 3CDZ | LEU | A | 749 | UNP | P00451 | | | SEE REMARK 999 |
| SEQADV | 3CDZ | LYS | A | 750 | UNP | P00451 | | | SEE REMARK 999 |
| SEQADV | 3CDZ | ARG | A | 751 | UNP | P00451 | | | SEE REMARK 999 |
| SEQADV | 3CDZ | HIS | A | 752 | UNP | P00451 | | | SEE REMARK 999 |
| SEQADV | 3CDZ | GLN | A | 753 | UNP | P00451 | | | SEE REMARK 999 |
| SEQADV | 3CDZ | ARG | A | 754 | UNP | P00451 | | | SEE REMARK 999 |
| SEQADV | 3CDZ | LEU | B | 1880 | UNP | P00451 | PHE | 1899 | VARIANT |

| | | | | | | | | | | | | | | | | |
|---|---|---|---|---|---|---|---|---|---|---|---|---|---|---|---|---|
| SEQRES | 1 | A | 754 | ALA | THR | ARG | ARG | TYR | TYR | LEU | GLY | ALA | VAL | GLU | LEU | SER |
| SEQRES | 2 | A | 754 | TRP | ASP | TYR | MET | GLN | SER | ASP | LEU | GLY | GLU | LEU | PRO | VAL |
| SEQRES | 3 | A | 754 | ASP | ALA | ARG | PHE | PRO | PRO | ARG | VAL | PRO | LYS | SER | PHE | PRO |
| SEQRES | 4 | A | 754 | PHE | ASN | THR | SER | VAL | VAL | TYR | LYS | LYS | THR | LEU | PHE | VAL |
| SEQRES | 5 | A | 754 | GLU | PHE | THR | ASP | HIS | LEU | PHE | ASN | ILE | ALA | LYS | PRO | ARG |
| SEQRES | 6 | A | 754 | PRO | PRO | TRP | MET | GLY | LEU | LEU | GLY | PRO | THR | ILE | GLN | ALA |
| SEQRES | 7 | A | 754 | GLU | VAL | TYR | ASP | THR | VAL | VAL | ILE | THR | LEU | LYS | ASN | MET |
| SEQRES | 8 | A | 754 | ALA | SER | HIS | PRO | VAL | SER | LEU | HIS | ALA | VAL | GLY | VAL | SER |
| SEQRES | 9 | A | 754 | TYR | TRP | LYS | ALA | SER | GLU | GLY | ALA | GLU | TYR | ASP | ASP | GLN |
| SEQRES | 10 | A | 754 | THR | SER | GLN | ARG | GLU | LYS | GLU | ASP | ASP | LYS | VAL | PHE | PRO |
| SEQRES | 11 | A | 754 | GLY | GLY | SER | HIS | THR | TYR | VAL | TRP | GLN | VAL | LEU | LYS | GLU |
| SEQRES | 12 | A | 754 | ASN | GLY | PRO | MET | ALA | SER | ASP | PRO | LEU | CYS | LEU | THR | TYR |
| SEQRES | 13 | A | 754 | SER | TYR | LEU | SER | HIS | VAL | ASP | LEU | VAL | LYS | ASP | LEU | ASN |
| SEQRES | 14 | A | 754 | SER | GLY | LEU | ILE | GLY | ALA | LEU | LEU | VAL | CYS | ARG | GLU | GLY |
| SEQRES | 15 | A | 754 | SER | LEU | ALA | LYS | GLU | LYS | THR | GLN | THR | LEU | HIS | LYS | PHE |
| SEQRES | 16 | A | 754 | ILE | LEU | LEU | PHE | ALA | VAL | PHE | ASP | GLU | GLY | LYS | SER | TRP |
| SEQRES | 17 | A | 754 | HIS | SER | GLU | THR | LYS | ASN | SER | LEU | MET | GLN | ASP | ARG | ASP |
| SEQRES | 18 | A | 754 | ALA | ALA | SER | ALA | ARG | ALA | TRP | PRO | LYS | MET | HIS | THR | VAL |
| SEQRES | 19 | A | 754 | ASN | GLY | TYR | VAL | ASN | ARG | SER | LEU | PRO | GLY | LEU | ILE | GLY |
| SEQRES | 20 | A | 754 | CYS | HIS | ARG | LYS | SER | VAL | TYR | TRP | HIS | VAL | ILE | GLY | MET |
| SEQRES | 21 | A | 754 | GLY | THR | THR | PRO | GLU | VAL | HIS | SER | ILE | PHE | LEU | GLU | GLY |
| SEQRES | 22 | A | 754 | HIS | THR | PHE | LEU | VAL | ARG | ASN | HIS | ARG | GLN | ALA | SER | LEU |
| SEQRES | 23 | A | 754 | GLU | ILE | SER | PRO | ILE | THR | PHE | LEU | THR | ALA | GLN | THR | LEU |
| SEQRES | 24 | A | 754 | LEU | MET | ASP | LEU | GLY | GLN | PHE | LEU | LEU | PHE | CYS | HIS | ILE |
| SEQRES | 25 | A | 754 | SER | SER | HIS | GLN | HIS | ASP | GLY | MET | GLU | ALA | TYR | VAL | LYS |
| SEQRES | 26 | A | 754 | VAL | ASP | SER | CYS | PRO | GLU | GLU | PRO | GLN | LEU | ARG | MET | LYS |
| SEQRES | 27 | A | 754 | ASN | ASN | GLU | GLU | ALA | GLU | ASP | TYR | ASP | ASP | ASP | LEU | THR |
| SEQRES | 28 | A | 754 | ASP | SER | GLU | MET | ASP | VAL | VAL | ARG | PHE | ASP | ASP | ASP | ASN |
| SEQRES | 29 | A | 754 | SER | PRO | SER | PHE | ILE | GLN | ILE | ARG | SER | VAL | ALA | LYS | LYS |
| SEQRES | 30 | A | 754 | HIS | PRO | LYS | THR | TRP | VAL | HIS | TYR | ILE | ALA | ALA | GLU | GLU |
| SEQRES | 31 | A | 754 | GLU | ASP | TRP | ASP | TYR | ALA | PRO | LEU | VAL | LEU | ALA | PRO | ASP |
| SEQRES | 32 | A | 754 | ASP | ARG | SER | TYR | LYS | SER | GLN | TYR | LEU | ASN | ASN | GLY | PRO |
| SEQRES | 33 | A | 754 | GLN | ARG | ILE | GLY | ARG | LYS | TYR | LYS | LYS | VAL | ARG | PHE | MET |
| SEQRES | 34 | A | 754 | ALA | TYR | THR | ASP | GLU | THR | PHE | LYS | THR | ARG | GLU | ALA | ILE |
| SEQRES | 35 | A | 754 | GLN | HIS | GLU | SER | GLY | ILE | LEU | GLY | PRO | LEU | LEU | TYR | GLY |
| SEQRES | 36 | A | 754 | GLU | VAL | GLY | ASP | THR | LEU | LEU | ILE | ILE | PHE | LYS | ASN | GLN |
| SEQRES | 37 | A | 754 | ALA | SER | ARG | PRO | TYR | ASN | ILE | TYR | PRO | HIS | GLY | ILE | THR |
| SEQRES | 38 | A | 754 | ASP | VAL | ARG | PRO | LEU | TYR | SER | ARG | ARG | LEU | PRO | LYS | GLY |
| SEQRES | 39 | A | 754 | VAL | LYS | HIS | LEU | LYS | ASP | PHE | PRO | ILE | LEU | PRO | GLY | GLU |
| SEQRES | 40 | A | 754 | ILE | PHE | LYS | TYR | LYS | TRP | THR | VAL | THR | VAL | GLU | ASP | GLY |
| SEQRES | 41 | A | 754 | PRO | THR | LYS | SER | ASP | PRO | ARG | CYS | LEU | THR | ARG | TYR | TYR |
| SEQRES | 42 | A | 754 | SER | SER | PHE | VAL | ASN | MET | GLU | ARG | ASP | LEU | ALA | SER | GLY |
| SEQRES | 43 | A | 754 | LEU | ILE | GLY | PRO | LEU | LEU | ILE | CYS | TYR | LYS | GLU | SER | VAL |
| SEQRES | 44 | A | 754 | ASP | GLN | ARG | GLY | ASN | GLN | ILE | MET | SER | ASP | LYS | ARG | ASN |
| SEQRES | 45 | A | 754 | VAL | ILE | LEU | PHE | SER | VAL | PHE | ASP | GLU | ASN | ARG | SER | TRP |
| SEQRES | 46 | A | 754 | TYR | LEU | THR | GLU | ASN | ILE | GLN | ARG | PHE | LEU | PRO | ASN | PRO |
| SEQRES | 47 | A | 754 | ALA | GLY | VAL | GLN | LEU | GLU | ASP | PRO | GLU | PHE | GLN | ALA | SER |
| SEQRES | 48 | A | 754 | ASN | ILE | MET | HIS | SER | ILE | ASN | GLY | TYR | VAL | PHE | ASP | SER |
| SEQRES | 49 | A | 754 | LEU | GLN | LEU | SER | VAL | CYS | LEU | HIS | GLU | VAL | ALA | TYR | TRP |
| SEQRES | 50 | A | 754 | TYR | ILE | LEU | SER | ILE | GLY | ALA | GLN | THR | ASP | PHE | LEU | SER |
| SEQRES | 51 | A | 754 | VAL | PHE | PHE | SER | GLY | TYR | THR | PHE | LYS | HIS | LYS | MET | VAL |
| SEQRES | 52 | A | 754 | TYR | GLU | ASP | THR | LEU | THR | LEU | PHE | PRO | PHE | SER | GLY | GLU |
| SEQRES | 53 | A | 754 | THR | VAL | PHE | MET | SER | MET | GLU | ASN | PRO | GLY | LEU | TRP | ILE |
| SEQRES | 54 | A | 754 | LEU | GLY | CYS | HIS | ASN | SER | ASP | PHE | ARG | ASN | ARG | GLY | MET |
| SEQRES | 55 | A | 754 | THR | ALA | LEU | LEU | LYS | VAL | SER | SER | CYS | ASP | LYS | ASN | THR |
| SEQRES | 56 | A | 754 | GLY | ASP | TYR | TYR | GLU | ASP | SER | TYR | GLU | ASP | ILE | SER | ALA |
| SEQRES | 57 | A | 754 | TYR | LEU | LEU | SER | LYS | ASN | ASN | ALA | ILE | GLU | PRO | ARG | SER |
| SEQRES | 58 | A | 754 | PHE | SER | GLN | ASN | PRO | PRO | VAL | LEU | LYS | ARG | HIS | GLN | ARG |
| SEQRES | 1 | B | 684 | GLU | ILE | THR | ARG | THR | THR | LEU | GLN | SER | ASP | GLN | GLU | GLU |
| SEQRES | 2 | B | 684 | ILE | ASP | TYR | ASP | ASP | THR | ILE | SER | VAL | GLU | MET | LYS | LYS |
| SEQRES | 3 | B | 684 | GLU | ASP | PHE | ASP | ILE | TYR | ASP | GLU | ASP | GLU | ASN | GLN | SER |
| SEQRES | 4 | B | 684 | PRO | ARG | SER | PHE | GLN | LYS | LYS | THR | ARG | HIS | TYR | PHE | ILE |
| SEQRES | 5 | B | 684 | ALA | ALA | VAL | GLU | ARG | LEU | TRP | ASP | TYR | GLY | MET | SER | SER |
| SEQRES | 6 | B | 684 | SER | PRO | HIS | VAL | LEU | ARG | ASN | ARG | ALA | GLN | SER | GLY | SER |
| SEQRES | 7 | B | 684 | VAL | PRO | GLN | PHE | LYS | LYS | VAL | VAL | PHE | GLN | GLU | PHE | THR |
| SEQRES | 8 | B | 684 | ASP | GLY | SER | PHE | THR | GLN | PRO | LEU | TYR | ARG | GLY | GLU | LEU |
| SEQRES | 9 | B | 684 | ASN | GLU | HIS | LEU | GLY | LEU | LEU | GLY | PRO | TYR | ILE | ARG | ALA |
| SEQRES | 10 | B | 684 | GLU | VAL | GLU | ASP | ASN | ILE | MET | VAL | THR | PHE | ARG | ASN | GLN |
| SEQRES | 11 | B | 684 | ALA | SER | ARG | PRO | TYR | SER | PHE | TYR | SER | SER | LEU | ILE | SER |
| SEQRES | 12 | B | 684 | TYR | GLU | GLU | ASP | GLN | ARG | GLN | GLY | ALA | GLU | PRO | ARG | LYS |
| SEQRES | 13 | B | 684 | ASN | PHE | VAL | LYS | PRO | ASN | GLU | THR | LYS | THR | TYR | PHE | TRP |
| SEQRES | 14 | B | 684 | LYS | VAL | GLN | HIS | HIS | MET | ALA | PRO | THR | LYS | ASP | GLU | PHE |
| SEQRES | 15 | B | 684 | ASP | CYS | LYS | ALA | TRP | ALA | TYR | PHE | SER | ASP | VAL | ASP | LEU |
| SEQRES | 16 | B | 684 | GLU | LYS | ASP | VAL | HIS | SER | GLY | LEU | ILE | GLY | PRO | LEU | LEU |
| SEQRES | 17 | B | 684 | VAL | CYS | HIS | THR | ASN | THR | LEU | ASN | PRO | ALA | HIS | GLY | ARG |
| SEQRES | 18 | B | 684 | GLN | VAL | THR | VAL | GLN | GLU | PHE | ALA | LEU | PHE | LEU | THR | ILE |
| SEQRES | 19 | B | 684 | PHE | ASP | GLU | THR | LYS | SER | TRP | TYR | PHE | THR | GLU | ASN | MET |
| SEQRES | 20 | B | 684 | GLU | ARG | ASN | CYS | ARG | ALA | PRO | CYS | ASN | ILE | GLN | MET | GLU |
| SEQRES | 21 | B | 684 | ASP | PRO | THR | PHE | LYS | GLU | ASN | TYR | ARG | PHE | HIS | ALA | ILE |
| SEQRES | 22 | B | 684 | ASN | GLY | TYR | ILE | MET | ASP | THR | LEU | PRO | GLY | LEU | VAL | MET |
| SEQRES | 23 | B | 684 | ALA | GLN | ASP | GLN | ARG | ILE | ARG | TRP | TYR | LEU | LEU | SER | MET |
| SEQRES | 24 | B | 684 | GLY | SER | ASN | GLU | ASN | ILE | HIS | SER | ILE | HIS | PHE | SER | GLY |
| SEQRES | 25 | B | 684 | HIS | VAL | PHE | THR | VAL | ARG | LYS | LYS | GLU | GLU | TYR | LYS | MET |
| SEQRES | 26 | B | 684 | ALA | LEU | TYR | ASN | LEU | TYR | PRO | GLY | VAL | PHE | GLU | THR | VAL |
| SEQRES | 27 | B | 684 | GLU | MET | LEU | PRO | SER | LYS | ALA | GLY | ILE | TRP | ARG | VAL | GLU |
| SEQRES | 28 | B | 684 | CYS | LEU | ILE | GLY | GLU | HIS | LEU | HIS | ALA | GLY | MET | SER | THR |
| SEQRES | 29 | B | 684 | LEU | PHE | LEU | VAL | TYR | SER | ASN | LYS | CYS | GLN | THR | PRO | LEU |
| SEQRES | 30 | B | 684 | GLY | MET | ALA | SER | GLY | HIS | ILE | ARG | ASP | PHE | GLN | ILE | THR |
| SEQRES | 31 | B | 684 | ALA | SER | GLY | GLN | TYR | GLY | GLN | TRP | ALA | PRO | LYS | LEU | ALA |
| SEQRES | 32 | B | 684 | ARG | LEU | HIS | TYR | SER | GLY | SER | ILE | ASN | ALA | TRP | SER | THR |
| SEQRES | 33 | B | 684 | LYS | GLU | PRO | PHE | SER | TRP | ILE | LYS | VAL | ASP | LEU | LEU | ALA |
| SEQRES | 34 | B | 684 | PRO | MET | ILE | ILE | HIS | GLY | ILE | LYS | THR | GLN | GLY | ALA | ARG |
| SEQRES | 35 | B | 684 | GLN | LYS | PHE | SER | SER | LEU | TYR | ILE | SER | GLN | PHE | ILE | ILE |
| SEQRES | 36 | B | 684 | MET | TYR | SER | LEU | ASP | GLY | LYS | LYS | TRP | GLN | THR | TYR | ARG |
| SEQRES | 37 | B | 684 | GLY | ASN | SER | THR | GLY | THR | LEU | MET | VAL | PHE | PHE | GLY | ASN |
| SEQRES | 38 | B | 684 | VAL | ASP | SER | SER | GLY | ILE | LYS | HIS | ASN | ILE | PHE | ASN | PRO |
| SEQRES | 39 | B | 684 | PRO | ILE | ILE | ALA | ARG | TYR | ILE | ARG | LEU | HIS | PRO | THR | HIS |
| SEQRES | 40 | B | 684 | TYR | SER | ILE | ARG | SER | THR | LEU | ARG | MET | GLU | LEU | MET | GLY |
| SEQRES | 41 | B | 684 | CYS | ASP | LEU | ASN | SER | CYS | SER | MET | PRO | LEU | GLY | MET | GLU |
| SEQRES | 42 | B | 684 | SER | LYS | ALA | ILE | SER | ASP | ALA | GLN | ILE | THR | ALA | SER | SER |
| SEQRES | 43 | B | 684 | TYR | PHE | THR | ASN | MET | PHE | ALA | THR | TRP | SER | PRO | SER | LYS |
| SEQRES | 44 | B | 684 | ALA | ARG | LEU | HIS | LEU | GLN | GLY | ARG | SER | ASN | ALA | TRP | ARG |
| SEQRES | 45 | B | 684 | PRO | GLN | VAL | ASN | ASN | PRO | LYS | GLU | TRP | LEU | GLN | VAL | ASP |
| SEQRES | 46 | B | 684 | PHE | GLN | LYS | THR | MET | LYS | VAL | THR | GLY | VAL | THR | THR | GLN |
| SEQRES | 47 | B | 684 | GLY | VAL | LYS | SER | LEU | LEU | THR | SER | MET | TYR | VAL | LYS | GLU |
| SEQRES | 48 | B | 684 | PHE | LEU | ILE | SER | SER | SER | GLN | ASP | GLY | HIS | GLN | TRP | THR |
| SEQRES | 49 | B | 684 | LEU | PHE | PHE | GLN | ASN | GLY | LYS | VAL | LYS | VAL | PHE | GLN | GLY |
| SEQRES | 50 | B | 684 | ASN | GLN | ASP | SER | PHE | THR | PRO | VAL | VAL | ASN | SER | LEU | ASP |
| SEQRES | 51 | B | 684 | PRO | PRO | LEU | LEU | THR | ARG | TYR | LEU | ARG | ILE | HIS | PRO | GLN |
| SEQRES | 52 | B | 684 | SER | TRP | VAL | HIS | GLN | ILE | ALA | LEU | ARG | MET | GLU | VAL | LEU |
| SEQRES | 53 | B | 684 | GLY | CYS | GLU | ALA | GLN | ASP | LEU | TYR | | | | | |

| | | | | | | |
|---|---|---|---|---|---|---|
| MODRES | 3CDZ | ASN | A | 239 | ASN | GLYCOSYLATION SITE |
| MODRES | 3CDZ | ASN | B | 1810 | ASN | GLYCOSYLATION SITE |
| MODRES | 3CDZ | ASN | B | 2118 | ASN | GLYCOSYLATION SITE |

| | | | | |
|---|---|---|---|---|
| HET | NAG | A | 755 | 14 |
| HET | NAG | A | 756 | 14 |
| HET | NAG | B | 2333 | 14 |
| HET | NAG | B | 2334 | 14 |
| HET | NAG | B | 2335 | 14 |
| HET | MAN | B | 2336 | 11 |
| HET | MAN | B | 2337 | 11 |
| HET | MAN | B | 2338 | 11 |
| HET | CU | A | 757 | 1 |
| HET | CA | A | 758 | 1 |
| HET | CU | B | 1 | 1 |

| | | |
|---|---|---|
| HETNAM | NAG | N-ACETYL-D-GLUCOSAMINE |
| HETNAM | MAN | ALPHA-D-MANNOSE |
| HETNAM | CU | COPPER (II) ION |
| HETNAM | CA | CALCIUM ION |
| HETSYN | NAG | NAG |

| FORMUL | 3 | NAG | 5(C8 H15 N O6) | | | | | | | |
|---|---|---|---|---|---|---|---|---|---|---|
| FORMUL | 6 | MAN | 3(C6 H12 O6) | | | | | | | |
| FORMUL | 7 | CU | 2(CU 2+) | | | | | | | |
| FORMUL | 8 | CA | CA 2+ | | | | | | | |
| HELIX | 1 | 1 | SER | A | 119 | GLU | A | 124 | 1 | 6 |
| HELIX | 2 | 2 | LEU | A | 141 | GLY | A | 145 | 5 | 5 |
| HELIX | 3 | 3 | ASP | A | 163 | LEU | A | 172 | 1 | 10 |
| HELIX | 4 | 4 | THR | A | 233 | TYR | A | 237 | 5 | 5 |
| HELIX | 5 | 5 | ILE | A | 312 | HIS | A | 317 | 1 | 6 |
| HELIX | 6 | 6 | THR | A | 516 | GLY | A | 520 | 5 | 5 |
| HELIX | 7 | 7 | MET | A | 539 | ALA | A | 544 | 1 | 6 |
| HELIX | 8 | 8 | ASN | A | 582 | SER | A | 584 | 5 | 3 |
| HELIX | 9 | 9 | THR | A | 588 | PHE | A | 594 | 1 | 7 |
| HELIX | 10 | 10 | ASP | A | 605 | SER | A | 611 | 1 | 7 |
| HELIX | 11 | 11 | GLU | B | 1844 | SER | B | 1849 | 1 | 6 |
| HELIX | 12 | 12 | TYR | B | 1890 | MET | B | 1895 | 1 | 6 |
| HELIX | 13 | 13 | ILE | B | 2002 | HIS | B | 2007 | 1 | 6 |
| HELIX | 14 | 14 | SER | B | 2186 | ALA | B | 2188 | 5 | 3 |
| HELIX | 15 | 15 | SER | B | 2204 | ALA | B | 2208 | 5 | 5 |

| | | | | | | | | | | | | | | | | | | |
|---|---|---|---|---|---|---|---|---|---|---|---|---|---|---|---|---|---|---|
| SHEET | 1 | A | 3 | ARG | A | 4 | TYR | A | 5 | 0 | | | | | | | | |
| SHEET | 2 | A | 3 | THR | A | 83 | LEU | A | 88 | 1 | O | VAL | A | 85 | N | TYR | A | 5 |
| SHEET | 3 | A | 3 | HIS | A | 134 | THR | A | 135 | −1 | O | HIS | A | 134 | N | LEU | A | 88 |
| SHEET | 1 | B | 3 | ARG | A | 4 | TYR | A | 5 | 0 | | | | | | | | |
| SHEET | 2 | B | 3 | THR | A | 83 | LEU | A | 88 | 1 | O | VAL | A | 85 | N | TYR | A | 5 |
| SHEET | 3 | B | 3 | TRP | A | 138 | GLN | A | 139 | −1 | O | TRP | A | 138 | N | VAL | A | 84 |
| SHEET | 1 | C | 3 | GLY | A | 73 | GLU | A | 79 | 0 | | | | | | | | |
| SHEET | 2 | C | 3 | GLY | A | 174 | CYS | A | 179 | 1 | O | LEU | A | 177 | N | ILE | A | 76 |
| SHEET | 3 | C | 3 | CYS | A | 153 | LEU | A | 154 | −1 | N | LEU | A | 154 | O | VAL | A | 178 |
| SHEET | 1 | D | 3 | GLY | A | 73 | GLU | A | 79 | 0 | | | | | | | | |
| SHEET | 2 | D | 3 | GLY | A | 174 | CYS | A | 179 | 1 | O | LEU | A | 177 | N | ILE | A | 76 |
| SHEET | 3 | D | 3 | SER | A | 157 | TYR | A | 158 | −1 | N | TYR | A | 158 | O | GLY | A | 174 |
| SHEET | 1 | E | 3 | LYS | A | 194 | LEU | A | 197 | 0 | | | | | | | | |
| SHEET | 2 | E | 3 | VAL | A | 253 | VAL | A | 257 | 1 | O | HIS | A | 256 | N | PHE | A | 195 |
| SHEET | 3 | E | 3 | THR | A | 295 | THR | A | 298 | −1 | O | ALA | A | 296 | N | TRP | A | 255 |
| SHEET | 1 | F | 5 | VAL | A | 426 | TYR | A | 431 | 0 | | | | | | | | |
| SHEET | 2 | F | 5 | THR | A | 381 | GLU | A | 390 | −1 | N | ALA | A | 387 | O | MET | A | 429 |
| SHEET | 3 | F | 5 | THR | A | 460 | GLN | A | 468 | 1 | O | LEU | A | 462 | N | TRP | A | 382 |
| SHEET | 4 | F | 5 | ILE | A | 508 | THR | A | 514 | −1 | O | TRP | A | 513 | N | LEU | A | 461 |
| SHEET | 5 | F | 5 | ASP | A | 482 | PRO | A | 485 | −1 | N | ARG | A | 484 | O | LYS | A | 512 |
| SHEET | 1 | G | 2 | TRP | A | 393 | ASP | A | 394 | 0 | | | | | | | | |
| SHEET | 2 | G | 2 | LYS | A | 422 | TYR | A | 423 | −1 | O | TYR | A | 423 | N | TRP | A | 393 |
| SHEET | 1 | H | 4 | LEU | A | 453 | GLU | A | 456 | 0 | | | | | | | | |
| SHEET | 2 | H | 4 | ILE | A | 548 | CYS | A | 554 | 1 | O | PRO | A | 550 | N | LEU | A | 453 |
| SHEET | 3 | H | 4 | CYS | A | 528 | SER | A | 534 | −1 | N | TYR | A | 533 | O | GLY | A | 549 |
| SHEET | 4 | H | 4 | TYR | A | 476 | HIS | A | 478 | −1 | N | HIS | A | 478 | O | TYR | A | 532 |
| SHEET | 1 | I | 4 | ILE | A | 613 | ILE | A | 617 | 0 | | | | | | | | |
| SHEET | 2 | I | 4 | ASN | A | 572 | ASP | A | 580 | −1 | N | PHE | A | 579 | O | MET | A | 614 |
| SHEET | 3 | I | 4 | VAL | A | 634 | ILE | A | 642 | 1 | O | LEU | A | 640 | N | PHE | A | 576 |
| SHEET | 4 | I | 4 | GLY | A | 675 | SER | A | 681 | −1 | O | GLU | A | 676 | N | ILE | A | 639 |
| SHEET | 1 | J | 2 | LEU | A | 649 | VAL | A | 651 | 0 | | | | | | | | |
| SHEET | 2 | J | 2 | LEU | A | 668 | LEU | A | 670 | −1 | O | LEU | A | 670 | N | LEU | A | 649 |
| SHEET | 1 | K | 4 | VAL | B | 1733 | GLU | B | 1737 | 0 | | | | | | | | |
| SHEET | 2 | K | 4 | THR | B | 1695 | GLU | B | 1704 | −1 | N | VAL | B | 1703 | O | VAL | B | 1734 |
| SHEET | 3 | K | 4 | ASP | B | 1769 | ASN | B | 1777 | 1 | O | ASN | B | 1770 | N | ARG | B | 1696 |
| SHEET | 4 | K | 4 | THR | B | 1812 | VAL | B | 1819 | −1 | O | TYR | B | 1815 | N | VAL | B | 1773 |
| SHEET | 1 | L | 3 | ILE | B | 1763 | ALA | B | 1765 | 0 | | | | | | | | |
| SHEET | 2 | L | 3 | ILE | B | 1852 | CYS | B | 1858 | 1 | O | CYS | B | 1858 | N | ALA | B | 1765 |
| SHEET | 3 | L | 3 | CYS | B | 1832 | PHE | B | 1838 | −1 | N | TRP | B | 1835 | O | LEU | B | 1855 |
| SHEET | 1 | M | 5 | GLU | B | 1875 | LEU | B | 1878 | 0 | | | | | | | | |
| SHEET | 2 | M | 5 | ILE | B | 1940 | LEU | B | 1945 | 1 | O | TYR | B | 1943 | N | PHE | B | 1876 |
| SHEET | 3 | M | 5 | PHE | B | 1983 | MET | B | 1988 | −1 | O | VAL | B | 1986 | N | TRP | B | 1942 |
| SHEET | 4 | M | 5 | PHE | B | 1963 | ARG | B | 1966 | −1 | N | THR | B | 1964 | O | GLU | B | 1987 |
| SHEET | 5 | M | 5 | GLU | B | 1970 | MET | B | 1973 | −1 | O | MET | B | 1973 | N | PHE | B | 1963 |
| SHEET | 1 | N | 5 | VAL | B | 1933 | ALA | B | 1935 | 0 | | | | | | | | |
| SHEET | 2 | N | 5 | THR | B | 2012 | TYR | B | 2017 | 1 | O | TYR | B | 2017 | N | MET | B | 1934 |
| SHEET | 3 | N | 5 | GLY | B | 1994 | CYS | B | 2000 | −1 | N | GLY | B | 1994 | O | VAL | B | 2016 |
| SHEET | 4 | N | 5 | HIS | B | 1954 | PHE | B | 1958 | −1 | N | HIS | B | 1957 | O | GLU | B | 1999 |
| SHEET | 5 | N | 5 | LEU | B | 1975 | LEU | B | 1978 | −1 | O | LEU | B | 1978 | N | HIS | B | 1954 |
| SHEET | 1 | O | 3 | THR | B | 2023 | PRO | B | 2024 | 0 | | | | | | | | |
| SHEET | 2 | O | 3 | SER | B | 2160 | CYS | B | 2169 | −1 | O | GLY | B | 2168 | N | THR | B | 2023 |
| SHEET | 3 | O | 3 | TRP | B | 2062 | THR | B | 2064 | −1 | N | TRP | B | 2062 | O | LEU | B | 2162 |
| SHEET | 1 | P | 6 | THR | B | 2023 | PRO | B | 2024 | 0 | | | | | | | | |
| SHEET | 2 | P | 6 | SER | B | 2160 | CYS | B | 2169 | −1 | O | GLY | B | 2168 | N | THR | B | 2023 |
| SHEET | 3 | P | 6 | ILE | B | 2071 | GLN | B | 2087 | −1 | N | GLY | B | 2083 | O | MET | B | 2167 |
| SHEET | 4 | P | 6 | ASN | B | 2138 | TYR | B | 2156 | −1 | O | ILE | B | 2144 | N | ILE | B | 2081 |
| SHEET | 5 | P | 6 | ILE | B | 2098 | SER | B | 2106 | −1 | N | SER | B | 2099 | O | HIS | B | 2155 |
| SHEET | 6 | P | 6 | VAL | B | 2125 | PHE | B | 2127 | −1 | O | PHE | B | 2126 | N | PHE | B | 2101 |
| SHEET | 1 | Q | 2 | ALA | B | 2089 | ARG | B | 2090 | 0 | | | | | | | | |
| SHEET | 2 | Q | 2 | SER | B | 2095 | LEU | B | 2096 | −1 | O | LEU | B | 2096 | N | ALA | B | 2089 |
| SHEET | 1 | R | 5 | ILE | B | 2190 | ALA | B | 2192 | 0 | | | | | | | | |
| SHEET | 2 | R | 5 | LEU | B | 2230 | GLN | B | 2246 | −1 | O | GLN | B | 2231 | N | THR | B | 2191 |
| SHEET | 3 | R | 5 | SER | B | 2296 | TRP | B | 2313 | −1 | O | LEU | B | 2297 | N | THR | B | 2241 |
| SHEET | 4 | R | 5 | VAL | B | 2257 | SER | B | 2265 | −1 | N | LEU | B | 2261 | O | HIS | B | 2309 |
| SHEET | 5 | R | 5 | PHE | B | 2283 | GLN | B | 2284 | −1 | O | PHE | B | 2283 | N | PHE | B | 2260 |
| SHEET | 1 | S | 5 | THR | B | 2272 | LEU | B | 2273 | 0 | | | | | | | | |
| SHEET | 2 | S | 5 | VAL | B | 2257 | SER | B | 2265 | −1 | N | SER | B | 2264 | O | THR | B | 2272 |
| SHEET | 3 | S | 5 | SER | B | 2296 | TRP | B | 2313 | −1 | O | HIS | B | 2309 | N | LEU | B | 2261 |
| SHEET | 4 | S | 5 | LEU | B | 2230 | GLN | B | 2246 | −1 | N | THR | B | 2241 | O | LEU | B | 2297 |
| SHEET | 5 | S | 5 | ARG | B | 2320 | CYS | B | 2326 | −1 | O | GLU | B | 2322 | N | THR | B | 2244 |
| SHEET | 1 | T | 2 | VAL | B | 2248 | LYS | B | 2249 | 0 | | | | | | | | |
| SHEET | 2 | T | 2 | SER | B | 2254 | MET | B | 2255 | −1 | O | MET | B | 2255 | N | VAL | B | 2248 |

| | | | | | | | | | | |
|---|---|---|---|---|---|---|---|---|---|---|
| SSBOND | 1 | CYS | A | 153 | CYS | A | 179 | 1555 | 1555 | 2.06 |
| SSBOND | 2 | CYS | A | 248 | CYS | A | 329 | 1555 | 1555 | 2.06 |
| SSBOND | 3 | CYS | A | 528 | CYS | A | 554 | 1555 | 1555 | 2.04 |
| SSBOND | 4 | CYS | A | 630 | CYS | A | 711 | 1555 | 1555 | 2.05 |
| SSBOND | 5 | CYS | B | 1832 | CYS | B | 1858 | 1555 | 1555 | 2.05 |
| SSBOND | 6 | CYS | B | 1899 | CYS | B | 1903 | 1555 | 1555 | 2.05 |
| SSBOND | 7 | CYS | B | 2021 | CYS | B | 2169 | 1555 | 1555 | 2.07 |
| SSBOND | 8 | CYS | B | 2174 | CYS | B | 2326 | 1555 | 1555 | 2.04 |

| | | | | | | | | | | | |
|---|---|---|---|---|---|---|---|---|---|---|---|
| LINK | O | LYS | A | 107 | CA | CA | A | 758 | 1555 | 1555 | 2.67 |
| LINK | O | GLU | A | 122 | CA | CA | A | 758 | 1555 | 1555 | 2.64 |
| LINK | OD1 | ASP | A | 125 | CA | CA | A | 758 | 1555 | 1555 | 2.77 |
| LINK | OD1 | ASP | A | 126 | CA | CA | A | 758 | 1555 | 1555 | 2.96 |
| LINK | OD2 | ASP | A | 126 | CA | CA | A | 758 | 1555 | 1555 | 2.73 |
| LINK | ND2 | ASN | A | 239 | C1 | NAG | A | 755 | 1555 | 1555 | 1.44 |
| LINK | ND1 | HIS | A | 267 | CU | CU | A | 757 | 1555 | 1555 | 2.05 |
| LINK | ND2 | ASN | B | 1810 | C1 | NAG | B | 2333 | 1555 | 1555 | 1.54 |
| LINK | ND1 | HIS | B | 1954 | CU | CU | B | 1 | 1555 | 1555 | 2.01 |
| LINK | ND1 | HIS | B | 2005 | CU | CU | B | 1 | 1555 | 1555 | 2.22 |
| LINK | ND2 | ASN | B | 2118 | C1 | NAG | B | 2334 | 1555 | 1555 | 1.25 |
| LINK | O4 | NAG | A | 755 | C1 | NAG | A | 756 | 1555 | 1555 | 1.47 |
| LINK | O4 | NAG | B | 2335 | C1 | MAN | B | 2336 | 1555 | 1555 | 1.46 |
| LINK | O3 | MAN | B | 2336 | C1 | MAN | B | 2337 | 1555 | 1555 | 1.47 |
| LINK | O6 | MAN | B | 2336 | C1 | MAN | B | 2338 | 1555 | 1555 | 1.45 |

| | | | | | | | | | | | | | | | |
|---|---|---|---|---|---|---|---|---|---|---|---|---|---|---|---|
| SITE | 1 | AC1 | 2 | ASN | A | 239 | HIS | A | 317 | | | | | | |
| SITE | 1 | AC2 | 1 | HIS | A | 317 | | | | | | | | | |
| SITE | 1 | AC3 | 2 | ALA | B | 1779 | ASN | B | 1810 | | | | | | |
| SITE | 1 | AC4 | 4 | GLN | B | 1870 | GLN | B | 1938 | ASN | B | 2118 | ASN | B | 2141 |
| SITE | 1 | AC5 | 3 | GLN | B | 1870 | THR | B | 1872 | GLN | B | 1938 | | | |
| SITE | 1 | AC7 | 2 | THR | B | 1872 | ARG | B | 1939 | | | | | | |
| SITE | 1 | AC8 | 5 | ILE | B | 1953 | HIS | B | 1954 | CYS | B | 2000 | ILE | B | 2002 |
| SITE | 2 | AC8 | 5 | HIS | B | 2005 | | | | | | | | | |
| SITE | 1 | AC9 | 3 | HIS | A | 267 | CYS | A | 310 | HIS | A | 315 | | | |
| SITE | 1 | BC1 | 8 | LYS | A | 107 | SER | A | 109 | GLU | A | 110 | ASP | A | 116 |
| SITE | 2 | BC1 | 8 | THR | A | 118 | GLU | A | 122 | ASP | A | 125 | ASP | A | 126 |

| | | | | | | | | |
|---|---|---|---|---|---|---|---|---|
| CRYST1 | 134.113 | 134.113 | 349.760 | 90.00 | 90.00 | 90.00 | P 41 21 2 | 8 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.007456 | 0.000000 | 0.000000 | 0.00000 |
| SCALE2 | 0.000000 | 0.007456 | 0.000000 | 0.00000 |
| SCALE3 | 0.000000 | 0.000000 | 0.002859 | 0.00000 |

| | | | | | | | | | | | |
|---|---|---|---|---|---|---|---|---|---|---|---|
| ATOM | 1 | N | ALA | A | 1 | −60.690 | −42.803 | 52.435 | 1.00 | 204.30 | N |
| ATOM | 2 | CA | ALA | A | 1 | −61.414 | −42.106 | 53.543 | 1.00 | 204.42 | C |
| ATOM | 3 | C | ALA | A | 1 | −60.692 | −40.816 | 53.993 | 1.00 | 204.42 | C |
| ATOM | 4 | O | ALA | A | 1 | −59.523 | −40.603 | 53.655 | 1.00 | 204.67 | O |
| ATOM | 5 | CB | ALA | A | 1 | −61.636 | −43.068 | 54.721 | 1.00 | 204.35 | C |
| ATOM | 6 | N | THR | A | 2 | −61.393 | −39.962 | 54.742 | 1.00 | 204.23 | N |
| ATOM | 7 | CA | THR | A | 2 | −60.868 | −38.659 | 55.184 | 1.00 | 203.98 | C |
| ATOM | 8 | C | THR | A | 2 | −61.548 | −38.223 | 56.491 | 1.00 | 204.17 | C |
| ATOM | 9 | O | THR | A | 2 | −62.645 | −37.667 | 56.451 | 1.00 | 204.36 | O |
| ATOM | 10 | CB | THR | A | 2 | −61.028 | −37.559 | 54.071 | 1.00 | 203.87 | C |
| ATOM | 11 | OG1 | THR | A | 2 | −61.136 | −36.266 | 54.674 | 1.00 | 203.37 | O |
| ATOM | 12 | CG2 | THR | A | 2 | −62.268 | −37.796 | 53.195 | 1.00 | 203.40 | C |
| ATOM | 13 | N | ARG | A | 3 | −60.899 | −38.461 | 57.638 | 1.00 | 204.25 | N |
| ATOM | 14 | CA | ARG | A | 3 | −61.542 | −38.318 | 58.982 | 1.00 | 204.23 | C |
| ATOM | 15 | C | ARG | A | 3 | −61.439 | −36.966 | 59.719 | 1.00 | 204.54 | C |
| ATOM | 16 | O | ARG | A | 3 | −60.380 | −36.335 | 59.729 | 1.00 | 204.66 | O |
| ATOM | 17 | CB | ARG | A | 3 | −61.091 | −39.446 | 59.929 | 1.00 | 204.18 | C |
| ATOM | 18 | CG | ARG | A | 3 | −62.238 | −40.326 | 60.423 | 1.00 | 203.86 | C |
| ATOM | 19 | CD | ARG | A | 3 | −61.761 | −41.580 | 61.142 | 1.00 | 203.49 | C |
| ATOM | 20 | NE | ARG | A | 3 | −61.639 | −42.715 | 60.230 | 1.00 | 201.88 | N |
| ATOM | 21 | CZ | ARG | A | 3 | −60.490 | −43.194 | 59.758 | 1.00 | 201.37 | C |
| ATOM | 22 | NH1 | ARG | A | 3 | −59.334 | −42.648 | 60.109 | 1.00 | 200.89 | N |
| ATOM | 23 | NH2 | ARG | A | 3 | −60.497 | −44.227 | 58.928 | 1.00 | 201.11 | N |
| ATOM | 24 | N | ARG | A | 4 | −62.545 | −36.551 | 60.350 | 1.00 | 204.69 | N |
| ATOM | 25 | CA | ARG | A | 4 | −62.625 | −35.282 | 61.094 | 1.00 | 204.93 | C |
| ATOM | 26 | C | ARG | A | 4 | −62.973 | −35.478 | 62.554 | 1.00 | 205.04 | C |
| ATOM | 27 | O | ARG | A | 4 | −63.835 | −36.286 | 62.876 | 1.00 | 205.05 | O |
| ATOM | 28 | CB | ARG | A | 4 | −63.671 | −34.357 | 60.480 | 1.00 | 204.91 | C |
| ATOM | 29 | CG | ARG | A | 4 | −63.122 | −33.420 | 59.432 | 1.00 | 205.50 | C |
| ATOM | 30 | CD | ARG | A | 4 | −64.234 | −32.631 | 58.772 | 1.00 | 206.19 | C |
| ATOM | 31 | NE | ARG | A | 4 | −63.852 | −32.171 | 57.438 | 1.00 | 206.84 | N |
| ATOM | 32 | CZ | ARG | A | 4 | −64.003 | −32.882 | 56.319 | 1.00 | 207.47 | C |
| ATOM | 33 | NH1 | ARG | A | 4 | −64.534 | −34.104 | 56.354 | 1.00 | 207.36 | N |
| ATOM | 34 | NH2 | ARG | A | 4 | −63.621 | −32.367 | 55.154 | 1.00 | 208.05 | N |
| ATOM | 35 | N | TYR | A | 5 | −62.318 | −34.714 | 63.427 | 1.00 | 205.38 | N |
| ATOM | 36 | CA | TYR | A | 5 | −62.560 | −34.775 | 64.875 | 1.00 | 205.77 | C |
| ATOM | 37 | C | TYR | A | 5 | −62.661 | −33.381 | 65.506 | 1.00 | 205.95 | C |
| ATOM | 38 | O | TYR | A | 5 | −61.690 | −32.880 | 66.080 | 1.00 | 206.09 | O |
| ATOM | 39 | CB | TYR | A | 5 | −61.481 | −35.616 | 65.573 | 1.00 | 205.84 | C |
| ATOM | 40 | CG | TYR | A | 5 | −61.590 | −37.100 | 65.299 | 1.00 | 206.26 | C |
| ATOM | 41 | CD1 | TYR | A | 5 | −60.469 | −37.855 | 64.953 | 1.00 | 206.47 | C |
| ATOM | 42 | CD2 | TYR | A | 5 | −62.826 | −37.753 | 65.377 | 1.00 | 206.99 | C |
| ATOM | 43 | CE1 | TYR | A | 5 | −60.579 | −39.234 | 64.701 | 1.00 | 206.84 | C |
| ATOM | 44 | CE2 | TYR | A | 5 | −62.951 | −39.123 | 65.121 | 1.00 | 207.07 | C |
| ATOM | 45 | CZ | TYR | A | 5 | −61.827 | −39.859 | 64.788 | 1.00 | 206.86 | C |
| ATOM | 46 | OH | TYR | A | 5 | −61.966 | −41.211 | 64.542 | 1.00 | 206.70 | O |
| ATOM | 47 | N | TYR | A | 6 | −63.856 | −32.786 | 65.424 | 1.00 | 206.08 | N |
| ATOM | 48 | CA | TYR | A | 6 | −64.098 | −31.350 | 65.716 | 1.00 | 206.04 | C |
| ATOM | 49 | C | TYR | A | 6 | −64.087 | −30.926 | 67.204 | 1.00 | 205.52 | C |
| ATOM | 50 | O | TYR | A | 6 | −64.929 | −30.127 | 67.634 | 1.00 | 205.23 | O |
| ATOM | 51 | CB | TYR | A | 6 | −65.415 | −30.879 | 65.038 | 1.00 | 206.58 | C |
| ATOM | 52 | CG | TYR | A | 6 | −65.313 | −30.496 | 63.553 | 1.00 | 207.40 | C |
| ATOM | 53 | CD1 | TYR | A | 6 | −64.059 | −30.373 | 62.914 | 1.00 | 208.36 | C |
| ATOM | 54 | CD2 | TYR | A | 6 | −66.467 | −30.217 | 62.800 | 1.00 | 207.45 | C |
| ATOM | 55 | CE1 | TYR | A | 6 | −63.957 | −30.007 | 61.562 | 1.00 | 208.40 | C |
| ATOM | 56 | CE2 | TYR | A | 6 | −66.374 | −29.849 | 61.444 | 1.00 | 207.94 | C |
| ATOM | 57 | CZ | TYR | A | 6 | −65.113 | −29.745 | 60.834 | 1.00 | 208.02 | C |
| ATOM | 58 | OH | TYR | A | 6 | −64.991 | −29.383 | 59.506 | 1.00 | 207.59 | O |
| ATOM | 59 | N | LEU | A | 7 | −63.106 | −31.416 | 67.966 | 1.00 | 205.09 | N |
| ATOM | 60 | CA | LEU | A | 7 | −63.156 | −31.336 | 69.436 | 1.00 | 204.73 | C |
| ATOM | 61 | C | LEU | A | 7 | −62.057 | −30.457 | 70.080 | 1.00 | 204.00 | C |
| ATOM | 62 | O | LEU | A | 7 | −60.859 | −30.734 | 69.951 | 1.00 | 203.63 | O |
| ATOM | 63 | CB | LEU | A | 7 | −63.222 | −32.765 | 70.064 | 1.00 | 205.11 | C |
| ATOM | 64 | CG | LEU | A | 7 | −63.949 | −33.971 | 69.376 | 1.00 | 205.77 | C |
| ATOM | 65 | CD1 | LEU | A | 7 | −63.797 | −35.313 | 70.103 | 1.00 | 205.38 | C |
| ATOM | 66 | CD2 | LEU | A | 7 | −65.436 | −33.746 | 69.077 | 1.00 | 206.70 | C |
| ATOM | 67 | N | GLY | A | 8 | −62.496 | −29.394 | 70.761 | 1.00 | 203.34 | N |
| ATOM | 68 | CA | GLY | A | 8 | −61.612 | −28.465 | 71.466 | 1.00 | 202.62 | C |
| ATOM | 69 | C | GLY | A | 8 | −61.194 | −28.930 | 72.849 | 1.00 | 202.23 | C |
| ATOM | 70 | O | GLY | A | 8 | −61.440 | −30.076 | 73.218 | 1.00 | 202.04 | O |
| ATOM | 71 | N | ALA | A | 9 | −60.563 | −28.035 | 73.612 | 1.00 | 201.98 | N |
| ATOM | 72 | CA | ALA | A | 9 | −60.038 | −28.342 | 74.959 | 1.00 | 201.79 | C |
| ATOM | 73 | C | ALA | A | 9 | −60.560 | −27.384 | 76.043 | 1.00 | 201.71 | C |
| ATOM | 74 | O | ALA | A | 9 | −60.650 | −26.174 | 75.810 | 1.00 | 201.79 | O |
| ATOM | 75 | CB | ALA | A | 9 | −58.520 | −28.348 | 74.953 | 1.00 | 201.78 | C |
| ATOM | 76 | N | VAL | A | 10 | −60.853 | −27.931 | 77.233 | 1.00 | 201.46 | N |
| ATOM | 77 | CA | VAL | A | 10 | −61.637 | −27.237 | 78.284 | 1.00 | 200.92 | C |
| ATOM | 78 | C | VAL | A | 10 | −61.035 | −27.245 | 79.706 | 1.00 | 200.61 | C |
| ATOM | 79 | O | VAL | A | 10 | −60.021 | −27.896 | 79.975 | 1.00 | 200.50 | O |
| ATOM | 80 | CB | VAL | A | 10 | −63.081 | −27.828 | 78.365 | 1.00 | 200.89 | C |
| ATOM | 81 | CG1 | VAL | A | 10 | −63.136 | −29.007 | 79.339 | 1.00 | 200.59 | C |
| ATOM | 82 | CG2 | VAL | A | 10 | −64.108 | −26.762 | 78.741 | 1.00 | 200.90 | C |
| ATOM | 83 | N | GLU | A | 11 | −61.695 | −26.502 | 80.597 | 1.00 | 200.33 | N |
| ATOM | 84 | CA | GLU | A | 11 | −61.451 | −26.515 | 82.040 | 1.00 | 200.13 | C |
| ATOM | 85 | C | GLU | A | 11 | −62.554 | −27.276 | 82.797 | 1.00 | 199.76 | C |
| ATOM | 86 | O | GLU | A | 11 | −63.739 | −26.906 | 82.728 | 1.00 | 199.92 | O |
| ATOM | 87 | CB | GLU | A | 11 | −61.400 | −25.085 | 82.576 | 1.00 | 200.27 | C |
| ATOM | 88 | CG | GLU | A | 11 | −60.023 | −24.473 | 82.656 | 1.00 | 200.60 | C |
| ATOM | 89 | CD | GLU | A | 11 | −60.011 | −23.235 | 83.526 | 1.00 | 201.04 | C |
| ATOM | 90 | OE1 | GLU | A | 11 | −60.833 | −22.321 | 83.284 | 1.00 | 201.38 | O |
| ATOM | 91 | OE2 | GLU | A | 11 | −59.183 | −23.182 | 84.458 | 1.00 | 201.26 | O |
| ATOM | 92 | N | LEU | A | 12 | −62.161 | −28.314 | 83.538 | 1.00 | 198.99 | N |
| ATOM | 93 | CA | LEU | A | 12 | −63.118 | −29.131 | 84.279 | 1.00 | 198.09 | C |
| ATOM | 94 | C | LEU | A | 12 | −62.673 | −29.376 | 85.719 | 1.00 | 197.56 | C |
| ATOM | 95 | O | LEU | A | 12 | −61.483 | −29.300 | 86.034 | 1.00 | 197.48 | O |
| ATOM | 96 | CB | LEU | A | 12 | −63.356 | −30.465 | 83.559 | 1.00 | 198.09 | C |
| ATOM | 97 | CG | LEU | A | 12 | −64.770 | −31.059 | 83.616 | 1.00 | 198.06 | C |
| ATOM | 98 | CD1 | LEU | A | 12 | −65.713 | −30.364 | 82.613 | 1.00 | 197.78 | C |
| ATOM | 99 | CD2 | LEU | A | 12 | −64.740 | −32.579 | 83.394 | 1.00 | 197.98 | C |
| ATOM | 100 | N | SER | A | 13 | −63.653 | −29.675 | 86.572 | 1.00 | 196.83 | N |
| ATOM | 101 | CA | SER | A | 13 | −63.454 | −29.974 | 87.994 | 1.00 | 195.94 | C |
| ATOM | 102 | C | SER | A | 13 | −63.478 | −31.498 | 88.285 | 1.00 | 195.46 | C |
| ATOM | 103 | O | SER | A | 13 | −64.406 | −32.198 | 87.868 | 1.00 | 195.36 | O |
| ATOM | 104 | CB | SER | A | 13 | −64.506 | −29.213 | 88.806 | 1.00 | 195.92 | C |
| ATOM | 105 | OG | SER | A | 13 | −65.557 | −28.741 | 87.968 | 1.00 | 195.33 | O |
| ATOM | 106 | N | TRP | A | 14 | −62.460 | −31.994 | 88.997 | 1.00 | 194.79 | N |
| ATOM | 107 | CA | TRP | A | 14 | −62.177 | −33.442 | 89.106 | 1.00 | 194.30 | C |
| ATOM | 108 | C | TRP | A | 14 | −62.241 | −33.989 | 90.542 | 1.00 | 194.28 | C |
| ATOM | 109 | O | TRP | A | 14 | −62.374 | −33.208 | 91.491 | 1.00 | 194.38 | O |
| ATOM | 110 | CB | TRP | A | 14 | −60.803 | −33.738 | 88.490 | 1.00 | 194.03 | C |
| ATOM | 111 | CG | TRP | A | 14 | −60.499 | −35.194 | 88.290 | 1.00 | 193.67 | C |
| ATOM | 112 | CD1 | TRP | A | 14 | −59.459 | −35.908 | 88.835 | 1.00 | 193.44 | C |
| ATOM | 113 | CD2 | TRP | A | 14 | −61.245 | −36.115 | 87.496 | 1.00 | 193.22 | C |
| ATOM | 114 | NE1 | TRP | A | 14 | −59.516 | −37.217 | 88.422 | 1.00 | 192.98 | N |
| ATOM | 115 | CE2 | TRP | A | 14 | −60.602 | −37.372 | 87.599 | 1.00 | 193.15 | C |
| ATOM | 116 | CE3 | TRP | A | 14 | −62.399 | −36.003 | 86.707 | 1.00 | 192.98 | C |
| ATOM | 117 | CZ2 | TRP | A | 14 | −61.076 | −38.508 | 86.940 | 1.00 | 193.36 | C |
| ATOM | 118 | CZ3 | TRP | A | 14 | −62.868 | −37.129 | 86.055 | 1.00 | 193.37 | C |
| ATOM | 119 | CH2 | TRP | A | 14 | −62.206 | −38.368 | 86.173 | 1.00 | 193.53 | C |
| ATOM | 120 | N | ASP | A | 15 | −62.136 | −35.320 | 90.696 | 1.00 | 194.11 | N |
| ATOM | 121 | CA | ASP | A | 15 | −62.258 | −35.980 | 92.011 | 1.00 | 193.87 | C |
| ATOM | 122 | C | ASP | A | 15 | −61.472 | −37.295 | 92.253 | 1.00 | 193.42 | C |
| ATOM | 123 | O | ASP | A | 15 | −60.231 | −37.308 | 92.298 | 1.00 | 193.24 | O |
| ATOM | 124 | CB | ASP | A | 15 | −63.743 | −36.191 | 92.344 | 1.00 | 194.09 | C |
| ATOM | 125 | CG | ASP | A | 15 | −64.002 | −36.293 | 93.847 | 1.00 | 195.40 | C |
| ATOM | 126 | OD1 | ASP | A | 15 | −63.400 | −35.507 | 94.615 | 1.00 | 196.58 | O |
| ATOM | 127 | OD2 | ASP | A | 15 | −64.815 | −37.153 | 94.267 | 1.00 | 197.17 | O |
| ATOM | 128 | N | TYR | A | 16 | −62.240 | −38.378 | 92.416 | 1.00 | 193.00 | N |
| ATOM | 129 | CA | TYR | A | 16 | −61.826 | −39.703 | 92.924 | 1.00 | 192.63 | C |
| ATOM | 130 | C | TYR | A | 16 | −60.897 | −40.462 | 91.985 | 1.00 | 192.47 | C |
| ATOM | 131 | O | TYR | A | 16 | −60.176 | −39.865 | 91.190 | 1.00 | 192.34 | O |
| ATOM | 132 | CB | TYR | A | 16 | −63.085 | −40.550 | 93.193 | 1.00 | 192.38 | C |
| ATOM | 133 | CG | TYR | A | 16 | −64.079 | −40.511 | 92.038 | 1.00 | 192.21 | C |
| ATOM | 134 | CD1 | TYR | A | 16 | −63.995 | −39.507 | 91.060 | 1.00 | 191.96 | C |
| ATOM | 135 | CD2 | TYR | A | 16 | −65.110 | −41.448 | 91.926 | 1.00 | 191.92 | C |
| ATOM | 136 | CE1 | TYR | A | 16 | −64.876 | −39.436 | 89.997 | 1.00 | 191.87 | C |
| ATOM | 137 | CE2 | TYR | A | 16 | −66.020 | −41.385 | 90.850 | 1.00 | 192.01 | C |
| ATOM | 138 | CZ | TYR | A | 16 | −65.884 | −40.368 | 89.887 | 1.00 | 192.21 | C |
| ATOM | 139 | OH | TYR | A | 16 | −66.735 | −40.250 | 88.807 | 1.00 | 192.15 | O |
| ATOM | 140 | N | VAL | A | 44 | −62.524 | −36.401 | 98.284 | 1.00 | 197.30 | N |
| ATOM | 141 | CA | VAL | A | 44 | −62.947 | −35.025 | 98.034 | 1.00 | 197.37 | C |
| ATOM | 142 | C | VAL | A | 44 | −61.748 | −34.037 | 97.897 | 1.00 | 197.56 | C |
| ATOM | 143 | O | VAL | A | 44 | −61.635 | −33.083 | 98.676 | 1.00 | 197.71 | O |
| ATOM | 144 | CB | VAL | A | 44 | −64.049 | −34.541 | 99.072 | 1.00 | 197.30 | C |
| ATOM | 145 | CG1 | VAL | A | 44 | −65.391 | −35.193 | 98.773 | 1.00 | 197.07 | C |
| ATOM | 146 | CG2 | VAL | A | 44 | −63.644 | −34.798 | 100.538 | 1.00 | 196.98 | C |
| ATOM | 147 | N | VAL | A | 45 | −60.874 | −34.270 | 96.899 | 1.00 | 197.58 | N |
| ATOM | 148 | CA | VAL | A | 45 | −59.650 | −33.439 | 96.659 | 1.00 | 197.43 | C |
| ATOM | 149 | C | VAL | A | 45 | −59.106 | −33.294 | 95.193 | 1.00 | 197.60 | C |
| ATOM | 150 | O | VAL | A | 45 | −58.700 | −34.293 | 94.570 | 1.00 | 197.84 | O |
| ATOM | 151 | CB | VAL | A | 45 | −58.450 | −33.866 | 97.591 | 1.00 | 197.43 | C |
| ATOM | 152 | CG1 | VAL | A | 45 | −58.375 | −32.988 | 98.837 | 1.00 | 197.37 | C |
| ATOM | 153 | CG2 | VAL | A | 45 | −58.500 | −35.359 | 97.948 | 1.00 | 196.96 | C |
| ATOM | 154 | N | TYR | A | 46 | −59.117 | −32.049 | 94.676 | 1.00 | 197.36 | N |
| ATOM | 155 | CA | TYR | A | 46 | −58.346 | −31.538 | 93.469 | 1.00 | 197.01 | C |
| ATOM | 156 | C | TYR | A | 46 | −58.992 | −31.364 | 92.063 | 1.00 | 196.07 | C |
| ATOM | 157 | O | TYR | A | 46 | −59.500 | −32.328 | 91.494 | 1.00 | 195.90 | O |
| ATOM | 158 | CB | TYR | A | 46 | −56.865 | −31.998 | 93.424 | 1.00 | 197.52 | C |
| ATOM | 159 | CG | TYR | A | 46 | −56.063 | −31.212 | 94.444 | 1.00 | 198.57 | C |
| ATOM | 160 | CD1 | TYR | A | 46 | −55.686 | −31.786 | 95.664 | 1.00 | 199.54 | C |
| ATOM | 161 | CD2 | TYR | A | 46 | −55.763 | −29.856 | 94.230 | 1.00 | 199.36 | C |
| ATOM | 162 | CE1 | TYR | A | 46 | −54.991 | −31.040 | 96.636 | 1.00 | 200.15 | C |
| ATOM | 163 | CE2 | TYR | A | 46 | −55.067 | −29.100 | 95.188 | 1.00 | 199.87 | C |
| ATOM | 164 | CZ | TYR | A | 46 | −54.685 | −29.697 | 96.391 | 1.00 | 199.79 | C |
| ATOM | 165 | OH | TYR | A | 46 | −54.002 | −28.957 | 97.340 | 1.00 | 199.26 | O |
| ATOM | 166 | N | LYS | A | 47 | −58.943 | −30.140 | 91.517 | 1.00 | 195.10 | N |
| ATOM | 167 | CA | LYS | A | 47 | −59.688 | −29.775 | 90.282 | 1.00 | 194.21 | C |
| ATOM | 168 | C | LYS | A | 47 | −58.866 | −29.055 | 89.169 | 1.00 | 194.07 | C |
| ATOM | 169 | O | LYS | A | 47 | −58.282 | −27.993 | 89.413 | 1.00 | 194.00 | O |
| ATOM | 170 | CB | LYS | A | 47 | −60.961 | −29.000 | 90.648 | 1.00 | 193.89 | C |
| ATOM | 171 | CG | LYS | A | 47 | −61.934 | −29.785 | 91.562 | 1.00 | 192.65 | C |
| ATOM | 172 | CD | LYS | A | 47 | −61.620 | −29.632 | 93.067 | 1.00 | 189.74 | C |
| ATOM | 173 | CE | LYS | A | 47 | −62.157 | −30.792 | 93.894 | 1.00 | 186.99 | C |
| ATOM | 174 | NZ | LYS | A | 47 | −61.567 | −30.791 | 95.253 | 1.00 | 184.74 | N |
| ATOM | 175 | N | LYS | A | 48 | −58.892 | −29.620 | 87.947 | 1.00 | 193.75 | N |
| ATOM | 176 | CA | LYS | A | 48 | −57.866 | −29.436 | 86.886 | 1.00 | 193.34 | C |
| ATOM | 177 | C | LYS | A | 48 | −58.322 | −28.734 | 85.588 | 1.00 | 193.67 | C |
| ATOM | 178 | O | LYS | A | 48 | −59.294 | −27.987 | 85.589 | 1.00 | 193.53 | O |
| ATOM | 179 | CB | LYS | A | 48 | −57.303 | −30.816 | 86.507 | 1.00 | 192.89 | C |
| ATOM | 180 | CG | LYS | A | 48 | −56.393 | −31.452 | 87.542 | 1.00 | 191.73 | C |
| ATOM | 181 | CD | LYS | A | 48 | −56.444 | −32.984 | 87.481 | 1.00 | 190.05 | C |
| ATOM | 182 | CE | LYS | A | 48 | −55.741 | −33.617 | 88.698 | 1.00 | 189.11 | C |
| ATOM | 183 | NZ | LYS | A | 48 | −56.054 | −35.055 | 88.950 | 1.00 | 187.75 | N |
| ATOM | 184 | N | THR | A | 49 | −57.565 | −28.957 | 84.501 | 1.00 | 194.25 | N |
| ATOM | 185 | CA | THR | A | 49 | −57.962 | −28.657 | 83.088 | 1.00 | 194.55 | C |
| ATOM | 186 | C | THR | A | 49 | −57.804 | −29.895 | 82.175 | 1.00 | 194.74 | C |
| ATOM | 187 | O | THR | A | 49 | −56.726 | −30.483 | 82.085 | 1.00 | 194.67 | O |
| ATOM | 188 | CB | THR | A | 49 | −57.235 | −27.408 | 82.443 | 1.00 | 194.53 | C |
| ATOM | 189 | OG1 | THR | A | 49 | −57.577 | −27.311 | 81.052 | 1.00 | 194.31 | O |
| ATOM | 190 | CG2 | THR | A | 49 | −55.724 | −27.497 | 82.546 | 1.00 | 194.35 | C |
| ATOM | 191 | N | LEU | A | 50 | −58.886 | −30.265 | 81.495 | 1.00 | 195.01 | N |
| ATOM | 192 | CA | LEU | A | 50 | −58.979 | −31.552 | 80.805 | 1.00 | 195.23 | C |
| ATOM | 193 | C | LEU | A | 50 | −58.939 | −31.361 | 79.286 | 1.00 | 195.39 | C |
| ATOM | 194 | O | LEU | A | 50 | −58.482 | −30.326 | 78.798 | 1.00 | 195.35 | O |
| ATOM | 195 | CB | LEU | A | 50 | −60.264 | −32.271 | 81.254 | 1.00 | 195.28 | C |
| ATOM | 196 | CG | LEU | A | 50 | −60.258 | −33.752 | 81.663 | 1.00 | 195.25 | C |
| ATOM | 197 | CD1 | LEU | A | 50 | −60.978 | −33.948 | 82.988 | 1.00 | 194.91 | C |
| ATOM | 198 | CD2 | LEU | A | 50 | −60.874 | −34.641 | 80.594 | 1.00 | 195.45 | C |
| ATOM | 199 | N | PHE | A | 51 | −59.411 | −32.360 | 78.549 | 1.00 | 195.61 | N |
| ATOM | 200 | CA | PHE | A | 51 | −59.375 | −32.340 | 77.101 | 1.00 | 195.99 | C |
| ATOM | 201 | C | PHE | A | 51 | −60.727 | −32.807 | 76.592 | 1.00 | 196.13 | C |
| ATOM | 202 | O | PHE | A | 51 | −60.989 | −34.003 | 76.555 | 1.00 | 196.01 | O |
| ATOM | 203 | CB | PHE | A | 51 | −58.291 | −33.303 | 76.614 | 1.00 | 196.20 | C |
| ATOM | 204 | CG | PHE | A | 51 | −57.347 | −32.710 | 75.596 | 1.00 | 196.80 | C |
| ATOM | 205 | CD1 | PHE | A | 51 | −55.967 | −32.816 | 75.771 | 1.00 | 196.93 | C |
| ATOM | 206 | CD2 | PHE | A | 51 | −57.828 | −32.050 | 74.463 | 1.00 | 197.01 | C |
| ATOM | 207 | CE1 | PHE | A | 51 | −55.085 | −32.274 | 74.842 | 1.00 | 196.72 | C |
| ATOM | 208 | CE2 | PHE | A | 51 | −56.951 | −31.504 | 73.526 | 1.00 | 196.57 | C |
| ATOM | 209 | CZ | PHE | A | 51 | −55.579 | −31.615 | 73.718 | 1.00 | 196.52 | C |
| ATOM | 210 | N | VAL | A | 52 | −61.582 | −31.869 | 76.194 | 1.00 | 196.56 | N |
| ATOM | 211 | CA | VAL | A | 52 | −62.976 | −32.203 | 75.864 | 1.00 | 197.10 | C |
| ATOM | 212 | C | VAL | A | 52 | −63.207 | −33.014 | 74.602 | 1.00 | 197.42 | C |
| ATOM | 213 | O | VAL | A | 52 | −62.271 | −33.432 | 73.935 | 1.00 | 197.55 | O |
| ATOM | 214 | CB | VAL | A | 52 | −63.927 | −30.993 | 75.839 | 1.00 | 197.18 | C |
| ATOM | 215 | CG1 | VAL | A | 52 | −64.629 | −30.857 | 77.172 | 1.00 | 197.21 | C |
| ATOM | 216 | CG2 | VAL | A | 52 | −63.211 | −29.728 | 75.432 | 1.00 | 197.43 | C |
| ATOM | 217 | N | GLU | A | 53 | −64.484 | −33.189 | 74.278 | 1.00 | 197.89 | N |
| ATOM | 218 | CA | GLU | A | 53 | −64.949 | −34.332 | 73.518 | 1.00 | 198.63 | C |
| ATOM | 219 | C | GLU | A | 53 | −66.030 | −33.957 | 72.475 | 1.00 | 198.77 | C |
| ATOM | 220 | O | GLU | A | 53 | −66.498 | −34.822 | 71.734 | 1.00 | 198.75 | O |
| ATOM | 221 | CB | GLU | A | 53 | −65.462 | −35.422 | 74.527 | 1.00 | 198.72 | C |
| ATOM | 222 | CG | GLU | A | 53 | −64.340 | −36.097 | 75.472 | 1.00 | 199.52 | C |
| ATOM | 223 | CD | GLU | A | 53 | −64.837 | −36.899 | 76.736 | 1.00 | 199.15 | C |
| ATOM | 224 | OE1 | GLU | A | 53 | −64.148 | −37.868 | 77.174 | 1.00 | 198.57 | O |
| ATOM | 225 | OE2 | GLU | A | 53 | −65.892 | −36.547 | 77.309 | 1.00 | 199.71 | O |
| ATOM | 226 | N | PHE | A | 54 | −66.377 | −32.669 | 72.380 | 1.00 | 199.27 | N |
| ATOM | 227 | CA | PHE | A | 54 | −67.732 | −32.256 | 71.916 | 1.00 | 199.86 | C |
| ATOM | 228 | C | PHE | A | 54 | −67.974 | −31.808 | 70.479 | 1.00 | 200.13 | C |
| ATOM | 229 | O | PHE | A | 54 | −67.100 | −31.259 | 69.836 | 1.00 | 200.00 | O |
| ATOM | 230 | CB | PHE | A | 54 | −68.403 | −31.279 | 72.909 | 1.00 | 199.97 | C |
| ATOM | 231 | CG | PHE | A | 54 | −67.644 | −29.998 | 73.152 | 1.00 | 200.18 | C |
| ATOM | 232 | CD1 | PHE | A | 54 | −66.270 | −29.907 | 72.940 | 1.00 | 200.60 | C |
| ATOM | 233 | CD2 | PHE | A | 54 | −68.312 | −28.884 | 73.652 | 1.00 | 200.54 | C |
| ATOM | 234 | CE1 | PHE | A | 54 | −65.584 | −28.714 | 73.190 | 1.00 | 201.06 | C |
| ATOM | 235 | CE2 | PHE | A | 54 | −67.637 | −27.685 | 73.908 | 1.00 | 200.79 | C |
| ATOM | 236 | CZ | PHE | A | 54 | −66.273 | −27.600 | 73.680 | 1.00 | 200.81 | C |
| ATOM | 237 | N | THR | A | 55 | −69.205 | −32.019 | 70.019 | 1.00 | 200.89 | N |
| ATOM | 238 | CA | THR | A | 55 | −69.608 | −31.834 | 68.620 | 1.00 | 201.87 | C |
| ATOM | 239 | C | THR | A | 55 | −69.748 | −30.376 | 68.160 | 1.00 | 202.86 | C |
| ATOM | 240 | O | THR | A | 55 | −70.491 | −30.084 | 67.212 | 1.00 | 202.87 | O |
| ATOM | 241 | CB | THR | A | 55 | −70.945 | −32.564 | 68.347 | 1.00 | 201.75 | C |
| ATOM | 242 | OG1 | THR | A | 55 | −71.351 | −32.330 | 66.995 | 1.00 | 201.67 | O |
| ATOM | 243 | CG2 | THR | A | 55 | −72.050 | −32.076 | 69.288 | 1.00 | 201.47 | C |
| ATOM | 244 | N | ASP | A | 56 | −69.005 | −29.477 | 68.805 | 1.00 | 204.16 | N |
| ATOM | 245 | CA | ASP | A | 56 | −69.223 | −28.020 | 68.684 | 1.00 | 205.24 | C |
| ATOM | 246 | C | ASP | A | 56 | −69.194 | −27.402 | 67.286 | 1.00 | 205.93 | C |
| ATOM | 247 | O | ASP | A | 56 | −68.605 | −27.935 | 66.330 | 1.00 | 205.95 | O |
| ATOM | 248 | CB | ASP | A | 56 | −68.320 | −27.208 | 69.676 | 1.00 | 205.23 | C |
| ATOM | 249 | CG | ASP | A | 56 | −67.003 | −26.619 | 69.036 | 1.00 | 205.31 | C |
| ATOM | 250 | OD1 | ASP | A | 56 | −66.244 | −25.943 | 69.774 | 1.00 | 204.74 | O |
| ATOM | 251 | OD2 | ASP | A | 56 | −66.703 | −26.811 | 67.834 | 1.00 | 205.26 | O |
| ATOM | 252 | N | HIS | A | 57 | −69.916 | −26.295 | 67.196 | 1.00 | 206.80 | N |
| ATOM | 253 | CA | HIS | A | 57 | −69.446 | −25.140 | 66.465 | 1.00 | 207.70 | C |
| ATOM | 254 | C | HIS | A | 57 | −69.302 | −24.033 | 67.506 | 1.00 | 208.25 | C |
| ATOM | 255 | O | HIS | A | 57 | −68.527 | −23.089 | 67.326 | 1.00 | 208.41 | O |
| ATOM | 256 | CB | HIS | A | 57 | −70.361 | −24.769 | 65.296 | 1.00 | 207.67 | C |
| ATOM | 257 | CG | HIS | A | 57 | −69.702 | −24.912 | 63.954 | 1.00 | 207.82 | C |
| ATOM | 258 | ND1 | HIS | A | 57 | −69.010 | −26.045 | 63.579 | 1.00 | 207.86 | N |
| ATOM | 259 | CD2 | HIS | A | 57 | −69.619 | −24.058 | 62.906 | 1.00 | 207.63 | C |
| ATOM | 260 | CE1 | HIS | A | 57 | −68.535 | −25.885 | 62.356 | 1.00 | 207.63 | C |
| ATOM | 261 | NE2 | HIS | A | 57 | −68.891 | −24.688 | 61.926 | 1.00 | 207.60 | N |
| ATOM | 262 | N | LEU | A | 58 | −70.027 | −24.191 | 68.615 | 1.00 | 208.93 | N |
| ATOM | 263 | CA | LEU | A | 58 | −69.854 | −23.341 | 69.795 | 1.00 | 209.60 | C |
| ATOM | 264 | C | LEU | A | 58 | −69.373 | −24.148 | 71.036 | 1.00 | 210.05 | C |
| ATOM | 265 | O | LEU | A | 58 | −68.232 | −24.627 | 71.077 | 1.00 | 209.89 | O |
| ATOM | 266 | CB | LEU | A | 58 | −71.133 | −22.510 | 70.094 | 1.00 | 209.59 | C |
| ATOM | 267 | CG | LEU | A | 58 | −71.581 | −21.269 | 69.286 | 1.00 | 209.35 | C |
| ATOM | 268 | CD1 | LEU | A | 58 | −72.976 | −20.800 | 69.720 | 1.00 | 208.78 | C |
| ATOM | 269 | CD2 | LEU | A | 58 | −70.593 | −20.105 | 69.375 | 1.00 | 209.26 | C |
| ATOM | 270 | N | PHE | A | 59 | −70.248 | −24.289 | 72.036 | 1.00 | 210.69 | N |
| ATOM | 271 | CA | PHE | A | 59 | −69.891 | −24.867 | 73.339 | 1.00 | 211.13 | C |
| ATOM | 272 | C | PHE | A | 59 | −71.025 | −25.779 | 73.891 | 1.00 | 211.30 | C |
| ATOM | 273 | O | PHE | A | 59 | −71.597 | −25.501 | 74.954 | 1.00 | 211.33 | O |
| ATOM | 274 | CB | PHE | A | 59 | −69.514 | −23.767 | 74.383 | 1.00 | 211.24 | C |
| ATOM | 275 | CG | PHE | A | 59 | −69.352 | −22.331 | 73.816 | 1.00 | 211.63 | C |
| ATOM | 276 | CD1 | PHE | A | 59 | −70.447 | −21.446 | 73.763 | 1.00 | 211.37 | C |
| ATOM | 277 | CD2 | PHE | A | 59 | −68.096 | −21.850 | 73.400 | 1.00 | 211.62 | C |
| ATOM | 278 | CE1 | PHE | A | 59 | −70.303 | −20.128 | 73.272 | 1.00 | 210.73 | C |
| ATOM | 279 | CE2 | PHE | A | 59 | −67.946 | −20.529 | 72.907 | 1.00 | 211.07 | C |
| ATOM | 280 | CZ | PHE | A | 59 | −69.052 | −19.674 | 72.847 | 1.00 | 210.85 | C |
| ATOM | 281 | N | ASN | A | 60 | −71.339 | −26.861 | 73.172 | 1.00 | 211.49 | N |
| ATOM | 282 | CA | ASN | A | 60 | −72.465 | −27.751 | 73.525 | 1.00 | 211.61 | C |
| ATOM | 283 | C | ASN | A | 60 | −72.066 | −29.187 | 73.902 | 1.00 | 211.88 | C |
| ATOM | 284 | O | ASN | A | 60 | −71.252 | −29.806 | 73.206 | 1.00 | 211.83 | O |
| ATOM | 285 | CB | ASN | A | 60 | −73.494 | −27.773 | 72.388 | 1.00 | 211.49 | C |
| ATOM | 286 | CG | ASN | A | 60 | −72.924 | −28.320 | 71.088 | 1.00 | 211.21 | C |
| ATOM | 287 | OD1 | ASN | A | 60 | −72.536 | −27.560 | 70.199 | 1.00 | 210.69 | O |
| ATOM | 288 | ND2 | ASN | A | 60 | −72.866 | −29.645 | 70.977 | 1.00 | 210.64 | N |
| ATOM | 289 | N | ILE | A | 61 | −72.665 | −29.712 | 74.980 | 1.00 | 212.29 | N |
| ATOM | 290 | CA | ILE | A | 61 | −72.336 | −31.053 | 75.553 | 1.00 | 212.61 | C |
| ATOM | 291 | C | ILE | A | 61 | −72.070 | −32.160 | 74.471 | 1.00 | 212.93 | C |
| ATOM | 292 | O | ILE | A | 61 | −72.537 | −32.017 | 73.331 | 1.00 | 213.00 | O |
| ATOM | 293 | CB | ILE | A | 61 | −73.363 | −31.500 | 76.728 | 1.00 | 212.48 | C |
| ATOM | 294 | CG1 | ILE | A | 61 | −74.842 | −31.430 | 76.301 | 1.00 | 212.07 | C |
| ATOM | 295 | CG2 | ILE | A | 61 | −73.141 | −30.680 | 78.016 | 1.00 | 211.87 | C |
| ATOM | 296 | CD1 | ILE | A | 61 | −75.831 | −32.081 | 77.277 | 1.00 | 211.96 | C |
| ATOM | 297 | N | ALA | A | 62 | −71.284 | −33.203 | 74.804 | 1.00 | 213.14 | N |
| ATOM | 298 | CA | ALA | A | 62 | −71.091 | −34.408 | 73.934 | 1.00 | 213.33 | C |
| ATOM | 299 | C | ALA | A | 62 | −70.118 | −35.446 | 74.488 | 1.00 | 213.78 | C |
| ATOM | 300 | O | ALA | A | 62 | −68.908 | −35.267 | 74.415 | 1.00 | 213.51 | O |
| ATOM | 301 | CB | ALA | A | 62 | −70.674 | −34.022 | 72.521 | 1.00 | 213.46 | C |
| ATOM | 302 | N | LYS | A | 63 | −70.653 | −36.556 | 74.988 | 1.00 | 214.60 | N |
| ATOM | 303 | CA | LYS | A | 63 | −69.885 | −37.442 | 75.875 | 1.00 | 215.58 | C |
| ATOM | 304 | C | LYS | A | 63 | −69.860 | −38.920 | 75.459 | 1.00 | 216.19 | C |
| ATOM | 305 | O | LYS | A | 63 | −70.919 | −39.519 | 75.258 | 1.00 | 215.92 | O |
| ATOM | 306 | CB | LYS | A | 63 | −70.414 | −37.323 | 77.315 | 1.00 | 215.67 | C |
| ATOM | 307 | CG | LYS | A | 63 | −70.265 | −35.931 | 77.963 | 1.00 | 216.05 | C |
| ATOM | 308 | CD | LYS | A | 63 | −71.475 | −35.525 | 78.838 | 1.00 | 216.31 | C |
| ATOM | 309 | CE | LYS | A | 63 | −72.526 | −34.723 | 78.045 | 1.00 | 216.52 | C |
| ATOM | 310 | NZ | LYS | A | 63 | −73.461 | −33.908 | 78.896 | 1.00 | 216.74 | N |
| ATOM | 311 | N | PRO | A | 64 | −68.640 | −39.495 | 75.290 | 1.00 | 217.05 | N |
| ATOM | 312 | CA | PRO | A | 64 | −68.376 | −40.943 | 75.143 | 1.00 | 217.49 | C |
| ATOM | 313 | C | PRO | A | 64 | −67.854 | −41.595 | 76.432 | 1.00 | 217.62 | C |
| ATOM | 314 | O | PRO | A | 64 | −67.745 | −42.829 | 76.538 | 1.00 | 217.40 | O |
| ATOM | 315 | CB | PRO | A | 64 | −67.256 | −40.975 | 74.073 | 1.00 | 217.61 | C |
| ATOM | 316 | CG | PRO | A | 64 | −66.477 | −39.657 | 74.270 | 1.00 | 217.40 | C |
| ATOM | 317 | CD | PRO | A | 64 | −67.387 | −38.722 | 75.122 | 1.00 | 217.36 | C |
| ATOM | 318 | N | ARG | A | 65 | −67.571 | −40.742 | 77.404 | 1.00 | 217.89 | N |
| ATOM | 319 | CA | ARG | A | 65 | −66.628 | −41.036 | 78.463 | 1.00 | 218.40 | C |
| ATOM | 320 | C | ARG | A | 65 | −66.985 | −42.129 | 79.493 | 1.00 | 218.80 | C |
| ATOM | 321 | O | ARG | A | 65 | −66.063 | −42.667 | 80.120 | 1.00 | 218.81 | O |
| ATOM | 322 | CB | ARG | A | 65 | −66.243 | −39.721 | 79.163 | 1.00 | 218.36 | C |
| ATOM | 323 | CG | ARG | A | 65 | −67.245 | −38.593 | 78.919 | 1.00 | 218.55 | C |
| ATOM | 324 | CD | ARG | A | 65 | −67.759 | −37.964 | 80.210 | 1.00 | 219.87 | C |
| ATOM | 325 | NE | ARG | A | 65 | −68.350 | −38.963 | 81.110 | 1.00 | 220.86 | N |
| ATOM | 326 | CZ | ARG | A | 65 | −69.391 | −38.757 | 81.919 | 1.00 | 220.96 | C |
| ATOM | 327 | NH1 | ARG | A | 65 | −70.001 | −37.575 | 81.958 | 1.00 | 221.33 | N |
| ATOM | 328 | NH2 | ARG | A | 65 | −69.833 | −39.749 | 82.686 | 1.00 | 220.42 | N |
| ATOM | 329 | N | PRO | A | 66 | −68.294 | −42.491 | 79.654 | 1.00 | 219.16 | N |
| ATOM | 330 | CA | PRO | A | 66 | −68.683 | −43.195 | 80.888 | 1.00 | 219.19 | C |
| ATOM | 331 | C | PRO | A | 66 | −67.620 | −44.084 | 81.547 | 1.00 | 219.22 | C |
| ATOM | 332 | O | PRO | A | 66 | −67.267 | −43.826 | 82.697 | 1.00 | 219.17 | O |
| ATOM | 333 | CB | PRO | A | 66 | −69.927 | −43.987 | 80.465 | 1.00 | 219.15 | C |
| ATOM | 334 | CG | PRO | A | 66 | −70.597 | −43.081 | 79.505 | 1.00 | 219.16 | C |
| ATOM | 335 | CD | PRO | A | 66 | −69.475 | −42.326 | 78.775 | 1.00 | 219.27 | C |
| ATOM | 336 | N | PRO | A | 67 | −67.074 | −45.086 | 80.827 | 1.00 | 219.32 | N |
| ATOM | 337 | CA | PRO | A | 67 | −66.296 | −46.010 | 81.633 | 1.00 | 219.28 | C |
| ATOM | 338 | C | PRO | A | 67 | −64.827 | −45.624 | 81.720 | 1.00 | 219.07 | C |
| ATOM | 339 | O | PRO | A | 67 | −64.477 | −44.520 | 82.152 | 1.00 | 218.73 | O |
| ATOM | 340 | CB | PRO | A | 67 | −66.464 | −47.342 | 80.883 | 1.00 | 219.33 | C |
| ATOM | 341 | CG | PRO | A | 67 | −66.767 | −46.950 | 79.436 | 1.00 | 219.49 | C |
| ATOM | 342 | CD | PRO | A | 67 | −67.048 | −45.463 | 79.398 | 1.00 | 219.37 | C |
| ATOM | 343 | N | TRP | A | 68 | −63.996 | −46.568 | 81.307 | 1.00 | 219.01 | N |
| ATOM | 344 | CA | TRP | A | 68 | −62.560 | −46.430 | 81.242 | 1.00 | 219.11 | C |
| ATOM | 345 | C | TRP | A | 68 | −62.040 | −45.048 | 80.844 | 1.00 | 219.00 | C |
| ATOM | 346 | O | TRP | A | 68 | −60.950 | −44.665 | 81.270 | 1.00 | 219.18 | O |
| ATOM | 347 | CB | TRP | A | 68 | −61.994 | −47.495 | 80.299 | 1.00 | 219.47 | C |
| ATOM | 348 | CG | TRP | A | 68 | −62.807 | −47.721 | 79.065 | 1.00 | 219.50 | C |
| ATOM | 349 | CD1 | TRP | A | 68 | −63.890 | −48.544 | 78.929 | 1.00 | 219.72 | C |
| ATOM | 350 | CD2 | TRP | A | 68 | −62.590 | −47.128 | 77.792 | 1.00 | 219.76 | C |
| ATOM | 351 | NE1 | TRP | A | 68 | −64.368 | −48.490 | 77.647 | 1.00 | 219.67 | N |
| ATOM | 352 | CE2 | TRP | A | 68 | −63.586 | −47.627 | 76.925 | 1.00 | 219.95 | C |
| ATOM | 353 | CE3 | TRP | A | 68 | −61.651 | −46.211 | 77.295 | 1.00 | 220.24 | C |
| ATOM | 354 | CZ2 | TRP | A | 68 | −63.671 | −47.241 | 75.582 | 1.00 | 220.41 | C |
| ATOM | 355 | CZ3 | TRP | A | 68 | −61.735 | −45.823 | 75.955 | 1.00 | 220.23 | C |
| ATOM | 356 | CH2 | TRP | A | 68 | −62.738 | −46.340 | 75.115 | 1.00 | 220.32 | C |
| ATOM | 357 | N | MET | A | 69 | −62.791 | −44.307 | 80.027 | 1.00 | 218.67 | N |
| ATOM | 358 | CA | MET | A | 69 | −62.392 | −42.934 | 79.704 | 1.00 | 218.16 | C |
| ATOM | 359 | C | MET | A | 69 | −62.372 | −42.136 | 81.012 | 1.00 | 217.46 | C |
| ATOM | 360 | O | MET | A | 69 | −63.405 | −41.975 | 81.678 | 1.00 | 217.42 | O |
| ATOM | 361 | CB | MET | A | 69 | −63.319 | −42.280 | 78.662 | 1.00 | 218.44 | C |
| ATOM | 362 | CG | MET | A | 69 | −63.249 | −42.840 | 77.226 | 1.00 | 218.89 | C |
| ATOM | 363 | SD | MET | A | 69 | −62.480 | −41.760 | 75.984 | 1.00 | 219.92 | S |
| ATOM | 364 | CE | MET | A | 69 | −63.374 | −42.198 | 74.481 | 1.00 | 218.86 | C |
| ATOM | 365 | N | GLY | A | 70 | −61.178 | −41.699 | 81.401 | 1.00 | 216.49 | N |
| ATOM | 366 | CA | GLY | A | 70 | −61.019 | −40.861 | 82.574 | 1.00 | 215.30 | C |
| ATOM | 367 | C | GLY | A | 70 | −60.907 | −39.447 | 82.076 | 1.00 | 214.50 | C |
| ATOM | 368 | O | GLY | A | 70 | −61.757 | −38.970 | 81.317 | 1.00 | 214.48 | O |
| ATOM | 369 | N | LEU | A | 71 | −59.852 | −38.772 | 82.497 | 1.00 | 213.69 | N |
| ATOM | 370 | CA | LEU | A | 71 | −59.497 | −37.523 | 81.873 | 1.00 | 213.01 | C |
| ATOM | 371 | C | LEU | A | 71 | −58.852 | −37.908 | 80.545 | 1.00 | 212.76 | C |
| ATOM | 372 | O | LEU | A | 71 | −57.677 | −38.287 | 80.495 | 1.00 | 212.93 | O |
| ATOM | 373 | CB | LEU | A | 71 | −58.519 | −36.735 | 82.734 | 1.00 | 212.85 | C |
| ATOM | 374 | CG | LEU | A | 71 | −58.291 | −37.189 | 84.167 | 1.00 | 212.35 | C |
| ATOM | 375 | CD1 | LEU | A | 71 | −57.152 | −38.174 | 84.186 | 1.00 | 211.59 | C |
| ATOM | 376 | CD2 | LEU | A | 71 | −57.963 | −35.987 | 85.021 | 1.00 | 212.53 | C |
| ATOM | 377 | N | LEU | A | 72 | −59.633 | −37.844 | 79.471 | 1.00 | 212.15 | N |
| ATOM | 378 | CA | LEU | A | 72 | −59.179 | −38.319 | 78.169 | 1.00 | 211.29 | C |
| ATOM | 379 | C | LEU | A | 72 | −59.874 | −37.627 | 77.018 | 1.00 | 210.75 | C |
| ATOM | 380 | O | LEU | A | 72 | −61.096 | −37.745 | 76.864 | 1.00 | 210.79 | O |
| ATOM | 381 | CB | LEU | A | 72 | −59.433 | −39.818 | 78.051 | 1.00 | 211.30 | C |
| ATOM | 382 | CG | LEU | A | 72 | −58.287 | −40.724 | 78.455 | 1.00 | 211.08 | C |
| ATOM | 383 | CD1 | LEU | A | 72 | −58.835 | −42.075 | 78.855 | 1.00 | 211.02 | C |
| ATOM | 384 | CD2 | LEU | A | 72 | −57.324 | −40.819 | 77.294 | 1.00 | 211.11 | C |
| ATOM | 385 | N | GLY | A | 73 | −59.098 | −36.909 | 76.211 | 1.00 | 209.97 | N |
| ATOM | 386 | CA | GLY | A | 73 | −59.580 | −36.476 | 74.909 | 1.00 | 209.12 | C |
| ATOM | 387 | C | GLY | A | 73 | −59.882 | −37.767 | 74.174 | 1.00 | 208.58 | C |
| ATOM | 388 | O | GLY | A | 73 | −59.135 | −38.745 | 74.336 | 1.00 | 208.57 | O |
| ATOM | 389 | N | PRO | A | 74 | −60.964 | −37.790 | 73.363 | 1.00 | 208.09 | N |
| ATOM | 390 | CA | PRO | A | 74 | −61.453 | −39.064 | 72.835 | 1.00 | 207.75 | C |
| ATOM | 391 | C | PRO | A | 74 | −60.366 | −39.854 | 72.118 | 1.00 | 207.32 | C |
| ATOM | 392 | O | PRO | A | 74 | −59.555 | −39.283 | 71.385 | 1.00 | 207.20 | O |
| ATOM | 393 | CB | PRO | A | 74 | −62.554 | −38.647 | 71.851 | 1.00 | 207.70 | C |
| ATOM | 394 | CG | PRO | A | 74 | −63.005 | −37.365 | 72.334 | 1.00 | 207.73 | C |
| ATOM | 395 | CD | PRO | A | 74 | −61.775 | −36.670 | 72.864 | 1.00 | 207.95 | C |
| ATOM | 396 | N | THR | A | 75 | −60.339 | −41.155 | 72.374 | 1.00 | 206.84 | N |
| ATOM | 397 | CA | THR | A | 75 | −59.483 | −42.061 | 71.643 | 1.00 | 206.42 | C |
| ATOM | 398 | C | THR | A | 75 | −59.780 | −41.818 | 70.162 | 1.00 | 205.95 | C |
| ATOM | 399 | O | THR | A | 75 | −60.878 | −42.126 | 69.686 | 1.00 | 205.93 | O |
| ATOM | 400 | CB | THR | A | 75 | −59.770 | −43.547 | 72.027 | 1.00 | 206.59 | C |
| ATOM | 401 | OG1 | THR | A | 75 | −60.044 | −43.659 | 73.433 | 1.00 | 206.74 | O |
| ATOM | 402 | CG2 | THR | A | 75 | −58.592 | −44.443 | 71.667 | 1.00 | 206.56 | C |
| ATOM | 403 | N | ILE | A | 76 | −58.828 | −41.206 | 69.455 | 1.00 | 205.35 | N |
| ATOM | 404 | CA | ILE | A | 76 | −58.976 | −40.974 | 68.012 | 1.00 | 204.58 | C |
| ATOM | 405 | C | ILE | A | 76 | −58.211 | −42.022 | 67.208 | 1.00 | 204.09 | C |
| ATOM | 406 | O | ILE | A | 76 | −57.035 | −42.298 | 67.475 | 1.00 | 203.93 | O |
| ATOM | 407 | CB | ILE | A | 76 | −58.592 | −39.534 | 67.568 | 1.00 | 204.54 | C |
| ATOM | 408 | CG1 | ILE | A | 76 | −57.090 | −39.285 | 67.677 | 1.00 | 204.55 | C |
| ATOM | 409 | CG2 | ILE | A | 76 | −59.369 | −38.494 | 68.361 | 1.00 | 204.50 | C |
| ATOM | 410 | CD1 | ILE | A | 76 | −56.619 | −38.153 | 66.782 | 1.00 | 205.11 | C |
| ATOM | 411 | N | GLN | A | 77 | −58.901 | −42.601 | 66.232 | 1.00 | 203.48 | N |
| ATOM | 412 | CA | GLN | A | 77 | −58.365 | −43.704 | 65.448 | 1.00 | 203.09 | C |
| ATOM | 413 | C | GLN | A | 77 | −58.323 | −43.387 | 63.959 | 1.00 | 202.67 | C |
| ATOM | 414 | O | GLN | A | 77 | −59.359 | −43.087 | 63.362 | 1.00 | 202.87 | O |
| ATOM | 415 | CB | GLN | A | 77 | −59.214 | −44.954 | 65.660 | 1.00 | 203.12 | C |
| ATOM | 416 | CG | GLN | A | 77 | −58.951 | −45.688 | 66.959 | 1.00 | 203.78 | C |
| ATOM | 417 | CD | GLN | A | 77 | −59.374 | −47.147 | 66.893 | 1.00 | 204.60 | C |
| ATOM | 418 | OE1 | GLN | A | 77 | −59.469 | −47.730 | 65.809 | 1.00 | 204.99 | O |
| ATOM | 419 | NE2 | GLN | A | 77 | −59.626 | −47.747 | 68.052 | 1.00 | 204.88 | N |
| ATOM | 420 | N | ALA | A | 78 | −57.133 | −43.467 | 63.361 | 1.00 | 201.96 | N |
| ATOM | 421 | CA | ALA | A | 78 | −56.980 | −43.328 | 61.910 | 1.00 | 201.33 | C |
| ATOM | 422 | C | ALA | A | 78 | −56.191 | −44.496 | 61.310 | 1.00 | 201.08 | C |
| ATOM | 423 | O | ALA | A | 78 | −55.274 | −45.031 | 61.948 | 1.00 | 201.07 | O |
| ATOM | 424 | CB | ALA | A | 78 | −56.332 | −42.007 | 61.567 | 1.00 | 201.21 | C |
| ATOM | 425 | N | GLU | A | 79 | −56.557 | −44.901 | 60.094 | 1.00 | 200.59 | N |
| ATOM | 426 | CA | GLU | A | 79 | −55.867 | −46.009 | 59.427 | 1.00 | 200.28 | C |
| ATOM | 427 | C | GLU | A | 79 | −54.764 | −45.538 | 58.459 | 1.00 | 199.88 | C |
| ATOM | 428 | O | GLU | A | 79 | −54.787 | −44.400 | 57.989 | 1.00 | 199.94 | O |
| ATOM | 429 | CB | GLU | A | 79 | −56.856 | −46.986 | 58.768 | 1.00 | 200.10 | C |
| ATOM | 430 | CG | GLU | A | 79 | −58.040 | −46.341 | 58.057 | 1.00 | 200.55 | C |
| ATOM | 431 | CD | GLU | A | 79 | −58.935 | −47.347 | 57.323 | 1.00 | 200.84 | C |
| ATOM | 432 | OE1 | GLU | A | 79 | −58.665 | −48.571 | 57.394 | 1.00 | 201.24 | O |
| ATOM | 433 | OE2 | GLU | A | 79 | −59.916 | −46.908 | 56.670 | 1.00 | 201.70 | O |
| ATOM | 434 | N | VAL | A | 80 | −53.808 | −46.429 | 58.183 | 1.00 | 199.39 | N |
| ATOM | 435 | CA | VAL | A | 80 | −52.572 | −46.144 | 57.429 | 1.00 | 198.73 | C |
| ATOM | 436 | C | VAL | A | 80 | −52.612 | −44.986 | 56.446 | 1.00 | 198.43 | C |
| ATOM | 437 | O | VAL | A | 80 | −51.681 | −44.191 | 56.421 | 1.00 | 198.46 | O |
| ATOM | 438 | CB | VAL | A | 80 | −52.047 | −47.384 | 56.647 | 1.00 | 198.74 | C |
| ATOM | 439 | CG1 | VAL | A | 80 | −51.193 | −48.281 | 57.524 | 1.00 | 198.73 | C |
| ATOM | 440 | CG2 | VAL | A | 80 | −53.185 | −48.155 | 56.035 | 1.00 | 198.71 | C |
| ATOM | 441 | N | TYR | A | 81 | −53.677 | −44.905 | 55.646 | 1.00 | 198.08 | N |
| ATOM | 442 | CA | TYR | A | 81 | −53.743 | −43.992 | 54.493 | 1.00 | 197.77 | C |
| ATOM | 443 | C | TYR | A | 81 | −54.557 | −42.703 | 54.670 | 1.00 | 197.40 | C |
| ATOM | 444 | O | TYR | A | 81 | −54.735 | −41.930 | 53.720 | 1.00 | 197.12 | O |
| ATOM | 445 | CB | TYR | A | 81 | −54.275 | −44.752 | 53.291 | 1.00 | 197.95 | C |
| ATOM | 446 | CG | TYR | A | 81 | −53.197 | −45.282 | 52.389 | 1.00 | 198.20 | C |
| ATOM | 447 | CD1 | TYR | A | 81 | −52.637 | −46.541 | 52.602 | 1.00 | 198.20 | C |
| ATOM | 448 | CD2 | TYR | A | 81 | −52.742 | −44.529 | 51.311 | 1.00 | 198.69 | C |
| ATOM | 449 | CE1 | TYR | A | 81 | −51.645 | −47.037 | 51.762 | 1.00 | 198.31 | C |
| ATOM | 450 | CE2 | TYR | A | 81 | −51.753 | −45.010 | 50.465 | 1.00 | 198.97 | C |
| ATOM | 451 | CZ | TYR | A | 81 | −51.208 | −46.263 | 50.697 | 1.00 | 198.69 | C |
| ATOM | 452 | OH | TYR | A | 81 | −50.228 | −46.735 | 49.853 | 1.00 | 198.90 | O |
| ATOM | 453 | N | ASP | A | 82 | −55.014 | −42.467 | 55.894 | 1.00 | 197.07 | N |
| ATOM | 454 | CA | ASP | A | 82 | −55.986 | −41.420 | 56.176 | 1.00 | 196.72 | C |
| ATOM | 455 | C | ASP | A | 82 | −55.428 | −40.061 | 56.588 | 1.00 | 196.22 | C |
| ATOM | 456 | O | ASP | A | 82 | −54.216 | −39.864 | 56.676 | 1.00 | 196.14 | O |
| ATOM | 457 | CB | ASP | A | 82 | −56.987 | −41.923 | 57.212 | 1.00 | 196.96 | C |
| ATOM | 458 | CG | ASP | A | 82 | −58.288 | −42.357 | 56.585 | 1.00 | 197.70 | C |
| ATOM | 459 | OD1 | ASP | A | 82 | −58.432 | −43.561 | 56.264 | 1.00 | 198.14 | O |
| ATOM | 460 | OD2 | ASP | A | 82 | −59.155 | −41.474 | 56.393 | 1.00 | 198.59 | O |
| ATOM | 461 | N | THR | A | 83 | −56.346 | −39.136 | 56.852 | 1.00 | 195.66 | N |
| ATOM | 462 | CA | THR | A | 83 | −56.032 | −37.729 | 57.042 | 1.00 | 195.15 | C |
| ATOM | 463 | C | THR | A | 83 | −56.998 | −37.114 | 58.055 | 1.00 | 194.96 | C |
| ATOM | 464 | O | THR | A | 83 | −58.164 | −36.872 | 57.730 | 1.00 | 194.97 | O |
| ATOM | 465 | CB | THR | A | 83 | −56.167 | −36.998 | 55.707 | 1.00 | 195.10 | C |
| ATOM | 466 | OG1 | THR | A | 83 | −57.190 | −37.642 | 54.933 | 1.00 | 194.78 | O |
| ATOM | 467 | CG2 | THR | A | 83 | −54.851 | −37.038 | 54.931 | 1.00 | 194.92 | C |
| ATOM | 468 | N | VAL | A | 84 | −56.508 | −36.864 | 59.273 | 1.00 | 194.63 | N |
| ATOM | 469 | CA | VAL | A | 84 | −57.350 | −36.439 | 60.411 | 1.00 | 194.25 | C |
| ATOM | 470 | C | VAL | A | 84 | −57.333 | −34.933 | 60.655 | 1.00 | 194.23 | C |
| ATOM | 471 | O | VAL | A | 84 | −56.279 | −34.304 | 60.610 | 1.00 | 194.25 | O |
| ATOM | 472 | CB | VAL | A | 84 | −56.987 | −37.220 | 61.709 | 1.00 | 194.09 | C |
| ATOM | 473 | CG1 | VAL | A | 84 | −57.255 | −36.399 | 62.968 | 1.00 | 193.70 | C |
| ATOM | 474 | CG2 | VAL | A | 84 | −57.748 | −38.526 | 61.753 | 1.00 | 193.73 | C |
| ATOM | 475 | N | VAL | A | 85 | −58.510 | −34.364 | 60.912 | 1.00 | 194.27 | N |
| ATOM | 476 | CA | VAL | A | 85 | −58.642 | −32.918 | 61.147 | 1.00 | 194.43 | C |
| ATOM | 477 | C | VAL | A | 85 | −59.309 | −32.525 | 62.479 | 1.00 | 194.58 | C |
| ATOM | 478 | O | VAL | A | 85 | −60.534 | −32.312 | 62.583 | 1.00 | 194.43 | O |
| ATOM | 479 | CB | VAL | A | 85 | −59.250 | −32.178 | 59.922 | 1.00 | 194.37 | C |
| ATOM | 480 | CG1 | VAL | A | 85 | −60.048 | −30.942 | 60.339 | 1.00 | 194.37 | C |
| ATOM | 481 | CG2 | VAL | A | 85 | −58.143 | −31.786 | 58.972 | 1.00 | 194.36 | C |
| ATOM | 482 | N | ILE | A | 86 | −58.457 | −32.444 | 63.496 | 1.00 | 194.76 | N |
| ATOM | 483 | CA | ILE | A | 86 | −58.813 | −31.888 | 64.789 | 1.00 | 194.90 | C |
| ATOM | 484 | C | ILE | A | 86 | −58.887 | −30.376 | 64.623 | 1.00 | 194.93 | C |
| ATOM | 485 | O | ILE | A | 86 | −58.006 | −29.784 | 64.003 | 1.00 | 195.24 | O |
| ATOM | 486 | CB | ILE | A | 86 | −57.730 | −32.206 | 65.856 | 1.00 | 194.89 | C |
| ATOM | 487 | CG1 | ILE | A | 86 | −57.240 | −33.658 | 65.732 | 1.00 | 195.05 | C |
| ATOM | 488 | CG2 | ILE | A | 86 | −58.251 | −31.892 | 67.264 | 1.00 | 195.07 | C |
| ATOM | 489 | CD1 | ILE | A | 86 | −55.961 | −33.957 | 66.492 | 1.00 | 194.90 | C |
| ATOM | 490 | N | THR | A | 87 | −59.940 | −29.755 | 65.147 | 1.00 | 194.78 | N |
| ATOM | 491 | CA | THR | A | 87 | −59.988 | −28.295 | 65.242 | 1.00 | 194.64 | C |
| ATOM | 492 | C | THR | A | 87 | −60.282 | −27.924 | 66.694 | 1.00 | 194.64 | C |
| ATOM | 493 | O | THR | A | 87 | −61.403 | −28.091 | 67.167 | 1.00 | 194.77 | O |
| ATOM | 494 | CB | THR | A | 87 | −61.034 | −27.675 | 64.289 | 1.00 | 194.52 | C |
| ATOM | 495 | OG1 | THR | A | 87 | −62.331 | −28.130 | 64.659 | 1.00 | 194.64 | O |
| ATOM | 496 | CG2 | THR | A | 87 | −60.790 | −28.082 | 62.846 | 1.00 | 194.45 | C |
| ATOM | 497 | N | LEU | A | 88 | −59.266 | −27.450 | 67.407 | 1.00 | 194.73 | N |
| ATOM | 498 | CA | LEU | A | 88 | −59.376 | −27.216 | 68.851 | 1.00 | 194.93 | C |
| ATOM | 499 | C | LEU | A | 88 | −59.940 | −25.827 | 69.172 | 1.00 | 195.13 | C |
| ATOM | 500 | O | LEU | A | 88 | −59.370 | −24.815 | 68.728 | 1.00 | 195.33 | O |
| ATOM | 501 | CB | LEU | A | 88 | −58.005 | −27.427 | 69.524 | 1.00 | 194.92 | C |
| ATOM | 502 | CG | LEU | A | 88 | −57.669 | −26.954 | 70.949 | 1.00 | 194.82 | C |
| ATOM | 503 | CD1 | LEU | A | 88 | −56.755 | −27.952 | 71.628 | 1.00 | 194.95 | C |
| ATOM | 504 | CD2 | LEU | A | 88 | −57.027 | −25.566 | 70.970 | 1.00 | 194.88 | C |
| ATOM | 505 | N | LYS | A | 89 | −61.057 | −25.781 | 69.919 | 1.00 | 195.00 | N |
| ATOM | 506 | CA | LYS | A | 89 | −61.559 | −24.517 | 70.495 | 1.00 | 194.65 | C |
| ATOM | 507 | C | LYS | A | 89 | −61.093 | −24.431 | 71.945 | 1.00 | 194.49 | C |
| ATOM | 508 | O | LYS | A | 89 | −61.497 | −25.243 | 72.778 | 1.00 | 194.60 | O |
| ATOM | 509 | CB | LYS | A | 89 | −63.090 | −24.351 | 70.359 | 1.00 | 194.38 | C |
| ATOM | 510 | CG | LYS | A | 89 | −63.536 | −22.863 | 70.355 | 1.00 | 194.37 | C |
| ATOM | 511 | CD | LYS | A | 89 | −64.749 | −22.573 | 69.441 | 1.00 | 194.01 | C |
| ATOM | 512 | CE | LYS | A | 89 | −64.794 | −21.100 | 68.943 | 1.00 | 193.18 | C |
| ATOM | 513 | NZ | LYS | A | 89 | −65.385 | −20.104 | 69.904 | 1.00 | 191.60 | N |
| ATOM | 514 | N | ASN | A | 90 | −60.208 | −23.472 | 72.222 | 1.00 | 194.28 | N |
| ATOM | 515 | CA | ASN | A | 90 | −59.578 | −23.332 | 73.536 | 1.00 | 194.09 | C |
| ATOM | 516 | C | ASN | A | 90 | −60.426 | −22.509 | 74.488 | 1.00 | 194.00 | C |
| ATOM | 517 | O | ASN | A | 90 | −60.933 | −21.446 | 74.138 | 1.00 | 193.98 | O |
| ATOM | 518 | CB | ASN | A | 90 | −58.166 | −22.728 | 73.423 | 1.00 | 194.15 | C |
| ATOM | 519 | CG | ASN | A | 90 | −57.378 | −22.778 | 74.743 | 1.00 | 194.01 | C |
| ATOM | 520 | OD1 | ASN | A | 90 | −56.400 | −22.042 | 74.925 | 1.00 | 193.58 | O |
| ATOM | 521 | ND2 | ASN | A | 90 | −57.791 | −23.654 | 75.656 | 1.00 | 194.01 | N |
| ATOM | 522 | N | MET | A | 91 | −60.579 | −23.033 | 75.696 | 1.00 | 193.88 | N |
| ATOM | 523 | CA | MET | A | 91 | −61.293 | −22.355 | 76.762 | 1.00 | 193.81 | C |
| ATOM | 524 | C | MET | A | 91 | −60.585 | −22.659 | 78.097 | 1.00 | 193.39 | C |
| ATOM | 525 | O | MET | A | 91 | −60.737 | −23.743 | 78.661 | 1.00 | 193.41 | O |
| ATOM | 526 | CB | MET | A | 91 | −62.806 | −22.706 | 76.737 | 1.00 | 193.89 | C |
| ATOM | 527 | CG | MET | A | 91 | −63.203 | −24.031 | 76.002 | 1.00 | 194.13 | C |
| ATOM | 528 | SD | MET | A | 91 | −64.789 | −24.071 | 75.073 | 1.00 | 194.52 | S |
| ATOM | 529 | CE | MET | A | 91 | −66.033 | −24.281 | 76.365 | 1.00 | 194.85 | C |
| ATOM | 530 | N | ALA | A | 92 | −59.774 | −21.701 | 78.555 | 1.00 | 192.90 | N |
| ATOM | 531 | CA | ALA | A | 92 | −58.919 | −21.837 | 79.746 | 1.00 | 192.40 | C |
| ATOM | 532 | C | ALA | A | 92 | −58.166 | −20.523 | 80.010 | 1.00 | 192.10 | C |
| ATOM | 533 | O | ALA | A | 92 | −57.985 | −19.726 | 79.094 | 1.00 | 192.22 | O |
| ATOM | 534 | CB | ALA | A | 92 | −57.919 | −22.994 | 79.566 | 1.00 | 192.27 | C |
| ATOM | 535 | N | SER | A | 93 | −57.720 | −20.305 | 81.250 | 1.00 | 191.67 | N |
| ATOM | 536 | CA | SER | A | 93 | −56.811 | −19.187 | 81.576 | 1.00 | 191.19 | C |
| ATOM | 537 | C | SER | A | 93 | −55.403 | −19.497 | 81.059 | 1.00 | 190.90 | C |
| ATOM | 538 | O | SER | A | 93 | −54.451 | −18.729 | 81.245 | 1.00 | 190.79 | O |
| ATOM | 539 | CB | SER | A | 93 | −56.783 | −18.920 | 83.090 | 1.00 | 191.23 | C |
| ATOM | 540 | OG | SER | A | 93 | −55.999 | −19.876 | 83.792 | 1.00 | 190.89 | O |
| ATOM | 541 | N | HIS | A | 94 | −55.313 | −20.636 | 80.384 | 1.00 | 190.52 | N |
| ATOM | 542 | CA | HIS | A | 94 | −54.075 | −21.240 | 79.961 | 1.00 | 190.16 | C |
| ATOM | 543 | C | HIS | A | 94 | −53.830 | −20.956 | 78.468 | 1.00 | 190.01 | C |
| ATOM | 544 | O | HIS | A | 94 | −54.773 | −20.946 | 77.666 | 1.00 | 189.98 | O |
| ATOM | 545 | CB | HIS | A | 94 | −54.199 | −22.751 | 80.191 | 1.00 | 190.06 | C |
| ATOM | 546 | CG | HIS | A | 94 | −53.003 | −23.378 | 80.833 | 1.00 | 189.71 | C |
| ATOM | 547 | ND1 | HIS | A | 94 | −53.111 | −24.321 | 81.829 | 1.00 | 189.27 | N |
| ATOM | 548 | CD2 | HIS | A | 94 | −51.677 | −23.208 | 80.617 | 1.00 | 189.73 | C |
| ATOM | 549 | CE1 | HIS | A | 94 | −51.903 | −24.703 | 82.202 | 1.00 | 189.55 | C |
| ATOM | 550 | NE2 | HIS | A | 94 | −51.015 | −24.043 | 81.481 | 1.00 | 189.51 | N |
| ATOM | 551 | N | PRO | A | 95 | −52.568 | −20.692 | 78.088 | 1.00 | 189.81 | N |
| ATOM | 552 | CA | PRO | A | 95 | −52.215 | −20.774 | 76.676 | 1.00 | 189.59 | C |
| ATOM | 553 | C | PRO | A | 95 | −51.887 | −22.224 | 76.346 | 1.00 | 189.41 | C |
| ATOM | 554 | O | PRO | A | 95 | −50.834 | −22.722 | 76.764 | 1.00 | 189.33 | O |
| ATOM | 555 | CB | PRO | A | 95 | −50.951 | −19.919 | 76.592 | 1.00 | 189.65 | C |
| ATOM | 556 | CG | PRO | A | 95 | −50.297 | −20.095 | 77.927 | 1.00 | 189.79 | C |
| ATOM | 557 | CD | PRO | A | 95 | −51.422 | −20.279 | 78.925 | 1.00 | 189.85 | C |
| ATOM | 558 | N | VAL | A | 96 | −52.770 | −22.909 | 75.626 | 1.00 | 189.24 | N |
| ATOM | 559 | CA | VAL | A | 96 | −52.564 | −24.350 | 75.424 | 1.00 | 189.25 | C |
| ATOM | 560 | C | VAL | A | 96 | −52.581 | −24.871 | 73.979 | 1.00 | 189.29 | C |
| ATOM | 561 | O | VAL | A | 96 | −53.446 | −24.513 | 73.177 | 1.00 | 189.17 | O |
| ATOM | 562 | CB | VAL | A | 96 | −53.481 | −25.206 | 76.332 | 1.00 | 189.24 | C |
| ATOM | 563 | CG1 | VAL | A | 96 | −52.942 | −25.237 | 77.762 | 1.00 | 188.99 | C |
| ATOM | 564 | CG2 | VAL | A | 96 | −54.904 | −24.696 | 76.298 | 1.00 | 189.43 | C |
| ATOM | 565 | N | SER | A | 97 | −51.614 | −25.744 | 73.693 | 1.00 | 189.43 | N |
| ATOM | 566 | CA | SER | A | 97 | −51.342 | −26.267 | 72.355 | 1.00 | 189.65 | C |
| ATOM | 567 | C | SER | A | 97 | −51.998 | −27.619 | 72.091 | 1.00 | 189.74 | C |
| ATOM | 568 | O | SER | A | 97 | −52.812 | −28.085 | 72.877 | 1.00 | 189.90 | O |
| ATOM | 569 | CB | SER | A | 97 | −49.835 | −26.442 | 72.179 | 1.00 | 189.76 | C |
| ATOM | 570 | OG | SER | A | 97 | −49.419 | −27.681 | 72.728 | 1.00 | 189.73 | O |
| ATOM | 571 | N | LEU | A | 98 | −51.618 | −28.234 | 70.972 | 1.00 | 189.77 | N |
| ATOM | 572 | CA | LEU | A | 98 | −52.000 | −29.599 | 70.619 | 1.00 | 190.00 | C |
| ATOM | 573 | C | LEU | A | 98 | −50.897 | −30.119 | 69.720 | 1.00 | 190.39 | C |
| ATOM | 574 | O | LEU | A | 98 | −50.558 | −29.484 | 68.719 | 1.00 | 190.65 | O |
| ATOM | 575 | CB | LEU | A | 98 | −53.356 | −29.635 | 69.897 | 1.00 | 189.83 | C |
| ATOM | 576 | CG | LEU | A | 98 | −54.007 | −30.925 | 69.362 | 1.00 | 189.39 | C |
| ATOM | 577 | CD1 | LEU | A | 98 | −53.636 | −31.207 | 67.917 | 1.00 | 188.78 | C |
| ATOM | 578 | CD2 | LEU | A | 98 | −53.721 | −32.131 | 70.230 | 1.00 | 188.69 | C |
| ATOM | 579 | N | HIS | A | 99 | −50.343 | −31.272 | 70.084 | 1.00 | 190.75 | N |
| ATOM | 580 | CA | HIS | A | 99 | −49.134 | −31.809 | 69.453 | 1.00 | 191.00 | C |
| ATOM | 581 | C | HIS | A | 99 | −49.187 | −33.330 | 69.466 | 1.00 | 191.25 | C |
| ATOM | 582 | O | HIS | A | 99 | −49.559 | −33.945 | 70.473 | 1.00 | 191.38 | O |
| ATOM | 583 | CB | HIS | A | 99 | −47.891 | −31.267 | 70.186 | 1.00 | 190.97 | C |
| ATOM | 584 | CG | HIS | A | 99 | −46.600 | −31.941 | 69.825 | 1.00 | 190.77 | C |
| ATOM | 585 | ND1 | HIS | A | 99 | −45.535 | −32.007 | 70.699 | 1.00 | 190.69 | N |
| ATOM | 586 | CD2 | HIS | A | 99 | −46.194 | −32.565 | 68.693 | 1.00 | 190.63 | C |
| ATOM | 587 | CE1 | HIS | A | 99 | −44.531 | −32.647 | 70.126 | 1.00 | 190.47 | C |
| ATOM | 588 | NE2 | HIS | A | 99 | −44.907 | −33.001 | 68.910 | 1.00 | 190.91 | N |
| ATOM | 589 | N | ALA | A | 100 | −48.829 | −33.936 | 68.340 | 1.00 | 191.55 | N |
| ATOM | 590 | CA | ALA | A | 100 | −48.905 | −35.390 | 68.219 | 1.00 | 191.89 | C |
| ATOM | 591 | C | ALA | A | 100 | −47.577 | −36.092 | 68.445 | 1.00 | 191.98 | C |
| ATOM | 592 | O | ALA | A | 100 | −46.548 | −35.475 | 68.721 | 1.00 | 192.10 | O |
| ATOM | 593 | CB | ALA | A | 100 | −49.462 | −35.785 | 66.859 | 1.00 | 192.00 | C |
| ATOM | 594 | N | VAL | A | 101 | −47.627 | −37.407 | 68.319 | 1.00 | 191.94 | N |
| ATOM | 595 | CA | VAL | A | 101 | −46.440 | −38.213 | 68.231 | 1.00 | 191.97 | C |
| ATOM | 596 | C | VAL | A | 101 | −46.801 | −39.337 | 67.250 | 1.00 | 192.25 | C |
| ATOM | 597 | O | VAL | A | 101 | −47.978 | −39.478 | 66.894 | 1.00 | 192.37 | O |
| ATOM | 598 | CB | VAL | A | 101 | −46.034 | −38.710 | 69.627 | 1.00 | 191.69 | C |
| ATOM | 599 | CG1 | VAL | A | 101 | −46.709 | −40.031 | 69.955 | 1.00 | 191.48 | C |
| ATOM | 600 | CG2 | VAL | A | 101 | −44.533 | −38.814 | 69.731 | 1.00 | 191.63 | C |
| ATOM | 601 | N | GLY | A | 102 | −45.803 | −40.088 | 66.773 | 1.00 | 192.40 | N |
| ATOM | 602 | CA | GLY | A | 102 | −46.030 | −41.273 | 65.922 | 1.00 | 192.29 | C |
| ATOM | 603 | C | GLY | A | 102 | −46.665 | −41.018 | 64.566 | 1.00 | 192.30 | C |
| ATOM | 604 | O | GLY | A | 102 | −46.938 | −41.959 | 63.809 | 1.00 | 192.10 | O |
| ATOM | 605 | N | VAL | A | 103 | −46.876 | −39.738 | 64.267 | 1.00 | 192.40 | N |
| ATOM | 606 | CA | VAL | A | 103 | −47.589 | −39.289 | 63.084 | 1.00 | 192.47 | C |
| ATOM | 607 | C | VAL | A | 103 | −46.757 | −38.220 | 62.407 | 1.00 | 192.68 | C |
| ATOM | 608 | O | VAL | A | 103 | −45.908 | −37.599 | 63.054 | 1.00 | 192.68 | O |
| ATOM | 609 | CB | VAL | A | 103 | −48.966 | −38.717 | 63.490 | 1.00 | 192.37 | C |
| ATOM | 610 | CG1 | VAL | A | 103 | −49.406 | −37.582 | 62.576 | 1.00 | 192.20 | C |
| ATOM | 611 | CG2 | VAL | A | 103 | −50.010 | −39.825 | 63.529 | 1.00 | 192.19 | C |
| ATOM | 612 | N | SER | A | 104 | −46.992 | −38.022 | 61.110 | 1.00 | 192.94 | N |
| ATOM | 613 | CA | SER | A | 104 | −46.374 | −36.913 | 60.382 | 1.00 | 193.37 | C |
| ATOM | 614 | C | SER | A | 104 | −47.349 | −35.757 | 60.108 | 1.00 | 193.39 | C |
| ATOM | 615 | O | SER | A | 104 | −48.572 | −35.938 | 60.108 | 1.00 | 193.34 | O |
| ATOM | 616 | CB | SER | A | 104 | −45.717 | −37.394 | 59.080 | 1.00 | 193.50 | C |
| ATOM | 617 | OG | SER | A | 104 | −46.640 | −37.415 | 58.004 | 1.00 | 194.00 | O |
| ATOM | 618 | N | TYR | A | 105 | −46.781 | −34.572 | 59.883 | 1.00 | 193.44 | N |
| ATOM | 619 | CA | TYR | A | 105 | −47.540 | −33.373 | 59.550 | 1.00 | 193.52 | C |
| ATOM | 620 | C | TYR | A | 105 | −46.616 | −32.322 | 58.993 | 1.00 | 193.55 | C |
| ATOM | 621 | O | TYR | A | 105 | −45.400 | −32.520 | 58.915 | 1.00 | 193.41 | O |
| ATOM | 622 | CB | TYR | A | 105 | −48.233 | −32.797 | 60.782 | 1.00 | 193.69 | C |
| ATOM | 623 | CG | TYR | A | 105 | −47.451 | −32.975 | 62.059 | 1.00 | 193.92 | C |
| ATOM | 624 | CD1 | TYR | A | 105 | −46.175 | −32.431 | 62.210 | 1.00 | 194.13 | C |
| ATOM | 625 | CD2 | TYR | A | 105 | −47.988 | −33.693 | 63.121 | 1.00 | 194.38 | C |
| ATOM | 626 | CE1 | TYR | A | 105 | −45.456 | −32.602 | 63.387 | 1.00 | 194.37 | C |
| ATOM | 627 | CE2 | TYR | A | 105 | −47.280 | −33.870 | 64.304 | 1.00 | 194.63 | C |
| ATOM | 628 | CZ | TYR | A | 105 | −46.017 | −33.324 | 64.431 | 1.00 | 194.43 | C |
| ATOM | 629 | OH | TYR | A | 105 | −45.325 | −33.510 | 65.603 | 1.00 | 194.42 | O |
| ATOM | 630 | N | TRP | A | 106 | −47.219 | −31.194 | 58.637 | 1.00 | 193.73 | N |
| ATOM | 631 | CA | TRP | A | 106 | −46.509 | −30.035 | 58.122 | 1.00 | 194.08 | C |
| ATOM | 632 | C | TRP | A | 106 | −46.126 | −29.122 | 59.253 | 1.00 | 193.76 | C |
| ATOM | 633 | O | TRP | A | 106 | −46.772 | −29.138 | 60.303 | 1.00 | 193.89 | O |
| ATOM | 634 | CB | TRP | A | 106 | −47.417 | −29.259 | 57.185 | 1.00 | 194.73 | C |
| ATOM | 635 | CG | TRP | A | 106 | −47.858 | −30.086 | 56.054 | 1.00 | 195.80 | C |
| ATOM | 636 | CD1 | TRP | A | 106 | −48.941 | −30.919 | 56.016 | 1.00 | 196.71 | C |
| ATOM | 637 | CD2 | TRP | A | 106 | −47.216 | −30.200 | 54.784 | 1.00 | 196.22 | C |
| ATOM | 638 | NE1 | TRP | A | 106 | −49.020 | −31.537 | 54.787 | 1.00 | 196.95 | N |
| ATOM | 639 | CE2 | TRP | A | 106 | −47.972 | −31.113 | 54.014 | 1.00 | 196.41 | C |
| ATOM | 640 | CE3 | TRP | A | 106 | −46.083 | −29.611 | 54.218 | 1.00 | 195.99 | C |
| ATOM | 641 | CZ2 | TRP | A | 106 | −47.630 | −31.449 | 52.711 | 1.00 | 195.95 | C |
| ATOM | 642 | CZ3 | TRP | A | 106 | −45.747 | −29.943 | 52.928 | 1.00 | 196.10 | C |
| ATOM | 643 | CH2 | TRP | A | 106 | −46.519 | −30.853 | 52.184 | 1.00 | 196.04 | C |
| ATOM | 644 | N | LYS | A | 107 | −45.095 | −28.308 | 59.028 | 1.00 | 193.26 | N |
| ATOM | 645 | CA | LYS | A | 107 | −44.673 | −27.309 | 60.006 | 1.00 | 192.85 | C |
| ATOM | 646 | C | LYS | A | 107 | −45.818 | −26.304 | 60.300 | 1.00 | 192.64 | C |
| ATOM | 647 | O | LYS | A | 107 | −45.717 | −25.478 | 61.216 | 1.00 | 192.67 | O |
| ATOM | 648 | CB | LYS | A | 107 | −43.346 | −26.645 | 59.582 | 1.00 | 192.67 | C |
| ATOM | 649 | CG | LYS | A | 107 | −43.295 | −26.267 | 57.992 | 1.00 | 193.04 | C |
| ATOM | 650 | CD | LYS | A | 107 | −41.590 | −26.137 | 57.659 | 1.00 | 192.92 | C |
| ATOM | 651 | CE | LYS | A | 107 | −41.425 | −25.416 | 56.277 | 1.00 | 192.02 | C |
| ATOM | 652 | NZ | LYS | A | 107 | −40.741 | −26.270 | 55.225 | 1.00 | 190.89 | N |
| ATOM | 653 | N | ALA | A | 108 | −46.918 | −26.428 | 59.544 | 1.00 | 192.34 | N |
| ATOM | 654 | CA | ALA | A | 108 | −48.195 | −25.713 | 59.800 | 1.00 | 191.91 | C |
| ATOM | 655 | C | ALA | A | 108 | −49.169 | −26.478 | 60.738 | 1.00 | 191.45 | C |
| ATOM | 656 | O | ALA | A | 108 | −50.381 | −26.209 | 60.799 | 1.00 | 191.16 | O |
| ATOM | 657 | CB | ALA | A | 108 | −48.875 | −25.355 | 58.485 | 1.00 | 192.15 | C |
| ATOM | 658 | N | SER | A | 109 | −48.606 | −27.455 | 61.436 | 1.00 | 190.85 | N |
| ATOM | 659 | CA | SER | A | 109 | −49.184 | −28.028 | 62.627 | 1.00 | 190.20 | C |
| ATOM | 660 | C | SER | A | 109 | −48.007 | −28.206 | 63.561 | 1.00 | 190.04 | C |
| ATOM | 661 | O | SER | A | 109 | −47.344 | −27.229 | 63.911 | 1.00 | 189.87 | O |
| ATOM | 662 | CB | SER | A | 109 | −49.880 | −29.349 | 62.337 | 1.00 | 190.06 | C |
| ATOM | 663 | OG | SER | A | 109 | −51.235 | −29.113 | 62.035 | 1.00 | 189.52 | O |
| ATOM | 664 | N | GLU | A | 110 | −47.712 | −29.450 | 63.925 | 1.00 | 189.86 | N |
| ATOM | 665 | CA | GLU | A | 110 | −46.697 | −29.746 | 64.936 | 1.00 | 189.67 | C |
| ATOM | 666 | C | GLU | A | 110 | −47.219 | −29.332 | 66.308 | 1.00 | 189.87 | C |
| ATOM | 667 | O | GLU | A | 110 | −47.307 | −30.168 | 67.205 | 1.00 | 190.07 | O |
| ATOM | 668 | CB | GLU | A | 110 | −45.363 | −29.063 | 64.619 | 1.00 | 189.39 | C |
| ATOM | 669 | CG | GLU | A | 110 | −44.195 | −29.531 | 65.452 | 1.00 | 188.30 | C |
| ATOM | 670 | CD | GLU | A | 110 | −42.939 | −28.743 | 65.165 | 1.00 | 187.60 | C |
| ATOM | 671 | OE1 | GLU | A | 110 | −43.044 | −27.579 | 64.736 | 1.00 | 188.15 | O |
| ATOM | 672 | OE2 | GLU | A | 110 | −41.838 | −29.281 | 65.365 | 1.00 | 186.83 | O |
| ATOM | 673 | N | GLY | A | 111 | −47.583 | −28.055 | 66.454 | 1.00 | 189.87 | N |
| ATOM | 674 | CA | GLY | A | 111 | −48.190 | −27.532 | 67.684 | 1.00 | 189.73 | C |
| ATOM | 675 | C | GLY | A | 111 | −47.163 | −27.351 | 68.780 | 1.00 | 189.67 | C |
| ATOM | 676 | O | GLY | A | 111 | −47.465 | −27.480 | 69.970 | 1.00 | 189.53 | O |
| ATOM | 677 | N | ALA | A | 112 | −45.942 | −27.040 | 68.361 | 1.00 | 189.69 | N |
| ATOM | 678 | CA | ALA | A | 112 | −44.803 | −27.000 | 69.256 | 1.00 | 189.81 | C |
| ATOM | 679 | C | ALA | A | 112 | −43.979 | −25.738 | 69.038 | 1.00 | 189.85 | C |
| ATOM | 680 | O | ALA | A | 112 | −43.345 | −25.566 | 67.993 | 1.00 | 189.82 | O |
| ATOM | 681 | CB | ALA | A | 112 | −43.956 | −28.224 | 69.039 | 1.00 | 189.94 | C |
| ATOM | 682 | N | GLU | A | 113 | −43.982 | −24.863 | 70.037 | 1.00 | 189.89 | N |
| ATOM | 683 | CA | GLU | A | 113 | −43.351 | −23.557 | 69.904 | 1.00 | 189.89 | C |
| ATOM | 684 | C | GLU | A | 113 | −41.853 | −23.640 | 70.158 | 1.00 | 189.61 | C |
| ATOM | 685 | O | GLU | A | 113 | −41.411 | −24.303 | 71.094 | 1.00 | 189.83 | O |
| ATOM | 686 | CB | GLU | A | 113 | −44.015 | −22.556 | 70.853 | 1.00 | 190.15 | C |
| ATOM | 687 | CG | GLU | A | 113 | −44.409 | −21.245 | 70.195 | 1.00 | 190.84 | C |
| ATOM | 688 | CD | GLU | A | 113 | −43.293 | −20.232 | 70.174 | 1.00 | 192.04 | C |
| ATOM | 689 | OE1 | GLU | A | 113 | −43.502 | −19.121 | 70.716 | 1.00 | 192.62 | O |
| ATOM | 690 | OE2 | GLU | A | 113 | −42.213 | −20.545 | 69.620 | 1.00 | 192.42 | O |
| ATOM | 691 | N | TYR | A | 114 | −41.097 | −22.962 | 69.302 | 1.00 | 189.17 | N |
| ATOM | 692 | CA | TYR | A | 114 | −39.633 | −22.892 | 69.307 | 1.00 | 189.00 | C |
| ATOM | 693 | C | TYR | A | 114 | −39.375 | −22.513 | 67.872 | 1.00 | 189.42 | C |
| ATOM | 694 | O | TYR | A | 114 | −40.266 | −22.679 | 67.035 | 1.00 | 189.46 | O |
| ATOM | 695 | CB | TYR | A | 114 | −38.956 | −24.229 | 69.658 | 1.00 | 188.40 | C |
| ATOM | 696 | CG | TYR | A | 114 | −38.970 | −25.252 | 68.548 | 1.00 | 187.67 | C |
| ATOM | 697 | CD1 | TYR | A | 114 | −37.791 | −25.679 | 67.956 | 1.00 | 187.49 | C |
| ATOM | 698 | CD2 | TYR | A | 114 | −40.170 | −25.791 | 68.083 | 1.00 | 187.40 | C |
| ATOM | 699 | CE1 | TYR | A | 114 | −37.809 | −26.622 | 66.914 | 1.00 | 187.33 | C |
| ATOM | 700 | CE2 | TYR | A | 114 | −40.198 | −26.729 | 67.048 | 1.00 | 186.88 | C |
| ATOM | 701 | CZ | TYR | A | 114 | −39.020 | −27.135 | 66.467 | 1.00 | 186.82 | C |
| ATOM | 702 | OH | TYR | A | 114 | −39.064 | −28.053 | 65.444 | 1.00 | 185.99 | O |
| ATOM | 703 | N | ASP | A | 115 | −38.191 | −22.002 | 67.564 | 1.00 | 190.04 | N |
| ATOM | 704 | CA | ASP | A | 115 | −37.987 | −21.417 | 66.237 | 1.00 | 190.72 | C |
| ATOM | 705 | C | ASP | A | 115 | −37.672 | −22.444 | 65.150 | 1.00 | 190.90 | C |
| ATOM | 706 | O | ASP | A | 115 | −36.522 | −22.598 | 64.728 | 1.00 | 190.80 | O |
| ATOM | 707 | CB | ASP | A | 115 | −36.955 | −20.276 | 66.264 | 1.00 | 190.97 | C |
| ATOM | 708 | CG | ASP | A | 115 | −35.533 | −20.774 | 66.451 | 1.00 | 191.59 | C |
| ATOM | 709 | OD1 | ASP | A | 115 | −35.275 | −21.498 | 67.439 | 1.00 | 192.19 | O |
| ATOM | 710 | OD2 | ASP | A | 115 | −34.676 | −20.443 | 65.603 | 1.00 | 192.14 | O |
| ATOM | 711 | N | ASP | A | 116 | −38.704 | −23.152 | 64.694 | 1.00 | 191.36 | N |
| ATOM | 712 | CA | ASP | A | 116 | −38.549 | −23.966 | 63.483 | 1.00 | 191.79 | C |
| ATOM | 713 | C | ASP | A | 116 | −38.657 | −23.053 | 62.247 | 1.00 | 192.15 | C |
| ATOM | 714 | O | ASP | A | 116 | −39.242 | −23.421 | 61.224 | 1.00 | 192.72 | O |
| ATOM | 715 | CB | ASP | A | 116 | −39.442 | −25.256 | 63.456 | 1.00 | 191.68 | C |
| ATOM | 716 | CG | ASP | A | 116 | −40.942 | −24.998 | 63.157 | 1.00 | 191.15 | C |
| ATOM | 717 | OD1 | ASP | A | 116 | −41.486 | −23.856 | 63.590 | 1.00 | 190.31 | O |
| ATOM | 718 | OD2 | ASP | A | 116 | −41.587 | −26.002 | 62.532 | 1.00 | 189.44 | O |
| ATOM | 719 | N | GLN | A | 117 | −38.065 | −21.860 | 62.377 | 1.00 | 192.07 | N |
| ATOM | 720 | CA | GLN | A | 117 | −37.977 | −20.840 | 61.327 | 1.00 | 191.91 | C |
| ATOM | 721 | C | GLN | A | 117 | −39.307 | −20.262 | 60.919 | 1.00 | 191.71 | C |
| ATOM | 722 | O | GLN | A | 117 | −39.487 | −19.052 | 60.936 | 1.00 | 191.69 | O |
| ATOM | 723 | CB | GLN | A | 117 | −37.230 | −21.344 | 60.098 | 1.00 | 192.01 | C |
| ATOM | 724 | CG | GLN | A | 117 | −36.487 | −20.229 | 59.420 | 1.00 | 192.85 | C |
| ATOM | 725 | CD | GLN | A | 117 | −35.716 | −19.378 | 60.417 | 1.00 | 193.67 | C |
| ATOM | 726 | OE1 | GLN | A | 117 | −35.262 | −19.875 | 61.454 | 1.00 | 194.05 | O |
| ATOM | 727 | NE2 | GLN | A | 117 | −35.570 | −18.090 | 60.112 | 1.00 | 194.18 | N |
| ATOM | 728 | N | THR | A | 118 | −40.198 | −21.151 | 60.498 | 1.00 | 191.67 | N |
| ATOM | 729 | CA | THR | A | 118 | −41.631 | −20.906 | 60.415 | 1.00 | 191.84 | C |
| ATOM | 730 | C | THR | A | 118 | −42.049 | −19.466 | 60.167 | 1.00 | 191.99 | C |
| ATOM | 731 | O | THR | A | 118 | −41.860 | −18.580 | 61.012 | 1.00 | 191.94 | O |
| ATOM | 732 | CB | THR | A | 118 | −42.387 | −21.460 | 61.665 | 1.00 | 191.93 | C |
| ATOM | 733 | OG1 | THR | A | 118 | −41.522 | −21.460 | 62.824 | 1.00 | 191.83 | O |
| ATOM | 734 | CG2 | THR | A | 118 | −42.859 | −22.874 | 61.395 | 1.00 | 192.11 | C |
| ATOM | 735 | N | SER | A | 119 | −42.642 | −19.252 | 58.998 | 1.00 | 192.24 | N |
| ATOM | 736 | CA | SER | A | 119 | −43.215 | −17.961 | 58.649 | 1.00 | 192.46 | C |
| ATOM | 737 | C | SER | A | 119 | −44.354 | −17.607 | 59.604 | 1.00 | 192.47 | C |
| ATOM | 738 | O | SER | A | 119 | −44.616 | −18.318 | 60.581 | 1.00 | 192.28 | O |
| ATOM | 739 | CB | SER | A | 119 | −43.689 | −17.948 | 57.189 | 1.00 | 192.49 | C |
| ATOM | 740 | OG | SER | A | 119 | −44.643 | −18.968 | 56.938 | 1.00 | 192.82 | O |
| ATOM | 741 | N | GLN | A | 120 | −45.022 | −16.500 | 59.321 | 1.00 | 192.61 | N |
| ATOM | 742 | CA | GLN | A | 120 | −46.008 | −15.983 | 60.234 | 1.00 | 192.80 | C |
| ATOM | 743 | C | GLN | A | 120 | −47.023 | −17.077 | 60.591 | 1.00 | 192.67 | C |
| ATOM | 744 | O | GLN | A | 120 | −47.082 | −17.537 | 61.736 | 1.00 | 192.43 | O |
| ATOM | 745 | CB | GLN | A | 120 | −46.670 | −14.733 | 59.641 | 1.00 | 192.95 | C |
| ATOM | 746 | CG | GLN | A | 120 | −46.968 | −13.648 | 60.673 | 1.00 | 193.95 | C |
| ATOM | 747 | CD | GLN | A | 120 | −47.927 | −14.116 | 61.778 | 1.00 | 195.45 | C |
| ATOM | 748 | OE1 | GLN | A | 120 | −47.497 | −14.543 | 62.857 | 1.00 | 196.26 | O |
| ATOM | 749 | NE2 | GLN | A | 120 | −49.228 | −14.052 | 61.502 | 1.00 | 195.68 | N |
| ATOM | 750 | N | ARG | A | 121 | −47.775 | −17.522 | 59.589 | 1.00 | 192.76 | N |
| ATOM | 751 | CA | ARG | A | 121 | −48.871 | −18.465 | 59.793 | 1.00 | 192.93 | C |
| ATOM | 752 | C | ARG | A | 121 | −48.414 | −19.865 | 60.191 | 1.00 | 193.01 | C |
| ATOM | 753 | O | ARG | A | 121 | −49.218 | −20.669 | 60.644 | 1.00 | 193.08 | O |
| ATOM | 754 | CB | ARG | A | 121 | −49.780 | −18.517 | 58.552 | 1.00 | 192.95 | C |
| ATOM | 755 | CG | ARG | A | 121 | −51.020 | −17.630 | 58.658 | 1.00 | 193.16 | C |
| ATOM | 756 | CD | ARG | A | 121 | −51.374 | −16.961 | 57.330 | 1.00 | 194.28 | C |
| ATOM | 757 | NE | ARG | A | 121 | −52.613 | −17.553 | 56.661 | 1.00 | 195.33 | N |
| ATOM | 758 | CZ | ARG | A | 121 | −52.793 | −17.153 | 55.291 | 1.00 | 196.40 | C |
| ATOM | 759 | NH1 | ARG | A | 121 | −52.078 | −16.154 | 54.436 | 1.00 | 196.49 | N |
| ATOM | 760 | NH2 | ARG | A | 121 | −53.966 | −17.764 | 54.802 | 1.00 | 196.78 | N |
| ATOM | 761 | N | GLU | A | 122 | −47.127 | −20.154 | 60.035 | 1.00 | 193.13 | N |
| ATOM | 762 | CA | GLU | A | 122 | −46.603 | −21.488 | 60.337 | 1.00 | 193.30 | C |
| ATOM | 763 | C | GLU | A | 122 | −46.290 | −21.693 | 61.828 | 1.00 | 193.14 | C |
| ATOM | 764 | O | GLU | A | 122 | −46.057 | −22.825 | 62.276 | 1.00 | 193.02 | O |
| ATOM | 765 | CB | GLU | A | 122 | −45.372 | −21.785 | 59.474 | 1.00 | 193.34 | C |
| ATOM | 766 | CG | GLU | A | 122 | −45.674 | −22.279 | 58.051 | 1.00 | 193.79 | C |
| ATOM | 767 | CD | GLU | A | 122 | −44.415 | −22.709 | 57.284 | 1.00 | 193.81 | C |
| ATOM | 768 | OE1 | GLU | A | 122 | −43.314 | −22.117 | 57.591 | 1.00 | 194.18 | O |
| ATOM | 769 | OE2 | GLU | A | 122 | −44.528 | −23.637 | 56.364 | 1.00 | 194.61 | O |
| ATOM | 770 | N | LYS | A | 123 | −46.289 | −20.595 | 62.586 | 1.00 | 193.09 | N |
| ATOM | 771 | CA | LYS | A | 123 | −46.055 | −20.637 | 64.033 | 1.00 | 192.99 | C |
| ATOM | 772 | C | LYS | A | 123 | −47.342 | −20.630 | 64.875 | 1.00 | 192.95 | C |
| ATOM | 773 | O | LYS | A | 123 | −47.330 | −21.071 | 66.027 | 1.00 | 192.94 | O |
| ATOM | 774 | CB | LYS | A | 123 | −45.142 | −19.487 | 64.472 | 1.00 | 193.01 | C |
| ATOM | 775 | CG | LYS | A | 123 | −43.689 | −19.878 | 64.668 | 1.00 | 192.89 | C |
| ATOM | 776 | CD | LYS | A | 123 | −42.907 | −18.790 | 65.411 | 1.00 | 192.77 | C |
| ATOM | 777 | CE | LYS | A | 123 | −41.406 | −19.033 | 65.350 | 1.00 | 192.90 | C |
| ATOM | 778 | NZ | LYS | A | 123 | −41.066 | −20.480 | 65.534 | 1.00 | 193.54 | N |
| ATOM | 779 | N | GLU | A | 124 | −48.442 | −20.138 | 64.302 | 1.00 | 192.75 | N |
| ATOM | 780 | CA | GLU | A | 124 | −49.723 | −20.028 | 65.018 | 1.00 | 192.48 | C |
| ATOM | 781 | C | GLU | A | 124 | −50.317 | −21.359 | 65.498 | 1.00 | 192.29 | C |
| ATOM | 782 | O | GLU | A | 124 | −51.225 | −21.370 | 66.325 | 1.00 | 192.43 | O |
| ATOM | 783 | CB | GLU | A | 124 | −50.740 | −19.268 | 64.174 | 1.00 | 192.40 | C |
| ATOM | 784 | CG | GLU | A | 124 | −50.442 | −17.788 | 64.065 | 1.00 | 192.64 | C |
| ATOM | 785 | CD | GLU | A | 124 | −51.327 | −17.095 | 63.052 | 1.00 | 192.93 | C |
| ATOM | 786 | OE1 | GLU | A | 124 | −51.631 | −17.712 | 62.008 | 1.00 | 192.99 | O |
| ATOM | 787 | OE2 | GLU | A | 124 | −51.718 | −15.934 | 63.297 | 1.00 | 192.93 | O |
| ATOM | 788 | N | ASP | A | 125 | −49.803 | −22.470 | 64.980 | 1.00 | 192.03 | N |
| ATOM | 789 | CA | ASP | A | 125 | −50.188 | −23.797 | 65.447 | 1.00 | 191.85 | C |
| ATOM | 790 | C | ASP | A | 125 | −49.508 | −24.101 | 66.767 | 1.00 | 191.97 | C |
| ATOM | 791 | O | ASP | A | 125 | −50.023 | −24.869 | 67.576 | 1.00 | 191.89 | O |
| ATOM | 792 | CB | ASP | A | 125 | −49.825 | −24.870 | 64.406 | 1.00 | 191.78 | C |
| ATOM | 793 | CG | ASP | A | 125 | −48.381 | −24.762 | 63.904 | 1.00 | 191.47 | C |
| ATOM | 794 | OD1 | ASP | A | 125 | −47.461 | −24.552 | 64.709 | 1.00 | 191.33 | O |
| ATOM | 795 | OD2 | ASP | A | 125 | −48.145 | −24.914 | 62.693 | 1.00 | 190.71 | O |
| ATOM | 796 | N | ASP | A | 126 | −48.354 | −23.467 | 66.969 | 1.00 | 192.21 | N |
| ATOM | 797 | CA | ASP | A | 126 | −47.427 | −23.765 | 68.068 | 1.00 | 192.61 | C |
| ATOM | 798 | C | ASP | A | 126 | −47.944 | −23.402 | 69.476 | 1.00 | 192.51 | C |
| ATOM | 799 | O | ASP | A | 126 | −47.542 | −24.019 | 70.474 | 1.00 | 192.47 | O |
| ATOM | 800 | CB | ASP | A | 126 | −46.069 | −23.084 | 67.804 | 1.00 | 192.86 | C |
| ATOM | 801 | CG | ASP | A | 126 | −45.298 | −23.691 | 66.611 | 1.00 | 193.84 | C |
| ATOM | 802 | OD1 | ASP | A | 126 | −44.508 | −22.953 | 65.963 | 1.00 | 194.58 | O |
| ATOM | 803 | OD2 | ASP | A | 126 | −45.462 | −24.903 | 66.317 | 1.00 | 194.65 | O |
| ATOM | 804 | N | LYS | A | 127 | −48.819 | −22.394 | 69.540 | 1.00 | 192.43 | N |
| ATOM | 805 | CA | LYS | A | 127 | −49.446 | −21.921 | 70.781 | 1.00 | 192.13 | C |
| ATOM | 806 | C | LYS | A | 127 | −50.819 | −21.332 | 70.472 | 1.00 | 192.17 | C |
| ATOM | 807 | O | LYS | A | 127 | −50.908 | −20.311 | 69.797 | 1.00 | 192.01 | O |
| ATOM | 808 | CB | LYS | A | 127 | −48.584 | −20.844 | 71.447 | 1.00 | 191.99 | C |
| ATOM | 809 | CG | LYS | A | 127 | −47.395 | −21.362 | 72.238 | 1.00 | 191.79 | C |
| ATOM | 810 | CD | LYS | A | 127 | −46.495 | −20.232 | 72.720 | 1.00 | 191.99 | C |
| ATOM | 811 | CE | LYS | A | 127 | −47.044 | −19.534 | 73.959 | 1.00 | 192.34 | C |
| ATOM | 812 | NZ | LYS | A | 127 | −48.298 | −18.762 | 73.709 | 1.00 | 192.51 | N |
| ATOM | 813 | N | VAL | A | 128 | −51.882 | −21.979 | 70.948 | 1.00 | 192.39 | N |
| ATOM | 814 | CA | VAL | A | 128 | −53.242 | −21.425 | 70.834 | 1.00 | 192.70 | C |
| ATOM | 815 | C | VAL | A | 128 | −53.510 | −20.451 | 71.985 | 1.00 | 192.91 | C |
| ATOM | 816 | O | VAL | A | 128 | −53.851 | −20.857 | 73.107 | 1.00 | 192.82 | O |
| ATOM | 817 | CB | VAL | A | 128 | −54.347 | −22.524 | 70.768 | 1.00 | 192.66 | C |
| ATOM | 818 | CG1 | VAL | A | 128 | −55.733 | −21.900 | 70.611 | 1.00 | 192.40 | C |
| ATOM | 819 | CG2 | VAL | A | 128 | −54.083 | −23.479 | 69.624 | 1.00 | 192.73 | C |
| ATOM | 820 | N | PHE | A | 129 | −53.342 | −19.165 | 71.684 | 1.00 | 193.21 | N |
| ATOM | 821 | CA | PHE | A | 129 | −53.482 | −18.093 | 72.655 | 1.00 | 193.69 | C |
| ATOM | 822 | C | PHE | A | 129 | −54.785 | −18.337 | 73.419 | 1.00 | 193.83 | C |
| ATOM | 823 | O | PHE | A | 129 | −55.742 | −18.833 | 72.818 | 1.00 | 193.83 | O |
| ATOM | 824 | CB | PHE | A | 129 | −53.496 | −16.713 | 71.969 | 1.00 | 194.01 | C |
| ATOM | 825 | CG | PHE | A | 129 | −52.789 | −16.655 | 70.611 | 1.00 | 195.17 | C |
| ATOM | 826 | CD1 | PHE | A | 129 | −53.173 | −15.696 | 69.660 | 1.00 | 196.03 | C |
| ATOM | 827 | CD2 | PHE | A | 129 | −51.744 | −17.533 | 70.276 | 1.00 | 196.06 | C |
| ATOM | 828 | CE1 | PHE | A | 129 | −52.534 | −15.614 | 68.397 | 1.00 | 195.96 | C |
| ATOM | 829 | CE2 | PHE | A | 129 | −51.105 | −17.465 | 69.008 | 1.00 | 196.01 | C |
| ATOM | 830 | CZ | PHE | A | 129 | −51.502 | −16.503 | 68.074 | 1.00 | 195.51 | C |
| ATOM | 831 | N | PRO | A | 130 | −54.831 | −17.988 | 74.735 | 1.00 | 194.00 | N |
| ATOM | 832 | CA | PRO | A | 130 | −55.907 | −18.383 | 75.680 | 1.00 | 193.82 | C |
| ATOM | 833 | C | PRO | A | 130 | −57.316 | −17.981 | 75.245 | 1.00 | 193.57 | C |
| ATOM | 834 | O | PRO | A | 130 | −57.604 | −16.793 | 75.092 | 1.00 | 193.41 | O |
| ATOM | 835 | CB | PRO | A | 130 | −55.527 | −17.650 | 76.980 | 1.00 | 193.79 | C |
| ATOM | 836 | CG | PRO | A | 130 | −54.627 | −16.533 | 76.549 | 1.00 | 193.93 | C |
| ATOM | 837 | CD | PRO | A | 130 | −53.838 | −17.127 | 75.408 | 1.00 | 194.16 | C |
| ATOM | 838 | N | GLY | A | 131 | −58.180 | −18.977 | 75.065 | 1.00 | 193.43 | N |
| ATOM | 839 | CA | GLY | A | 131 | −59.510 | −18.755 | 74.508 | 1.00 | 193.44 | C |
| ATOM | 840 | C | GLY | A | 131 | −59.453 | −18.486 | 73.013 | 1.00 | 193.49 | C |
| ATOM | 841 | O | GLY | A | 131 | −60.184 | −17.638 | 72.496 | 1.00 | 193.52 | O |
| ATOM | 842 | N | GLY | A | 132 | −58.585 | −19.218 | 72.316 | 1.00 | 193.53 | N |
| ATOM | 843 | CA | GLY | A | 132 | −58.349 | −19.006 | 70.888 | 1.00 | 193.52 | C |
| ATOM | 844 | C | GLY | A | 132 | −59.250 | −19.801 | 69.961 | 1.00 | 193.50 | C |
| ATOM | 845 | O | GLY | A | 132 | −60.462 | −19.556 | 69.902 | 1.00 | 193.56 | O |
| ATOM | 846 | N | SER | A | 133 | −58.641 | −20.750 | 69.242 | 1.00 | 193.40 | N |
| ATOM | 847 | CA | SER | A | 133 | −59.274 | −21.529 | 68.168 | 1.00 | 193.19 | C |
| ATOM | 848 | C | SER | A | 133 | −58.250 | −21.757 | 67.045 | 1.00 | 193.11 | C |
| ATOM | 849 | O | SER | A | 133 | −57.655 | −20.799 | 66.527 | 1.00 | 193.18 | O |
| ATOM | 850 | CB | SER | A | 133 | −60.515 | −20.810 | 67.607 | 1.00 | 193.20 | C |
| ATOM | 851 | OG | SER | A | 133 | −61.514 | −21.722 | 67.188 | 1.00 | 192.93 | O |
| ATOM | 852 | N | HIS | A | 134 | −58.039 | −23.023 | 66.682 | 1.00 | 192.83 | N |
| ATOM | 853 | CA | HIS | A | 134 | −57.167 | −23.383 | 65.545 | 1.00 | 192.56 | C |
| ATOM | 854 | C | HIS | A | 134 | −57.476 | −24.781 | 64.955 | 1.00 | 192.14 | C |
| ATOM | 855 | O | HIS | A | 134 | −57.864 | −25.707 | 65.686 | 1.00 | 192.31 | O |
| ATOM | 856 | CB | HIS | A | 134 | −55.677 | −23.249 | 65.928 | 1.00 | 192.67 | C |
| ATOM | 857 | CG | HIS | A | 134 | −54.729 | −23.475 | 64.787 | 1.00 | 192.90 | C |
| ATOM | 858 | ND1 | HIS | A | 134 | −53.709 | −24.402 | 64.839 | 1.00 | 192.91 | N |
| ATOM | 859 | CD2 | HIS | A | 134 | −54.654 | −22.901 | 63.562 | 1.00 | 192.99 | C |
| ATOM | 860 | CE1 | HIS | A | 134 | −53.045 | −24.386 | 63.696 | 1.00 | 192.94 | C |
| ATOM | 861 | NE2 | HIS | A | 134 | −53.599 | −23.484 | 62.905 | 1.00 | 192.98 | N |
| ATOM | 862 | N | THR | A | 135 | −57.314 | −24.905 | 63.632 | 1.00 | 191.25 | N |
| ATOM | 863 | CA | THR | A | 135 | −57.494 | −26.162 | 62.892 | 1.00 | 190.26 | C |
| ATOM | 864 | C | THR | A | 135 | −56.205 | −26.980 | 62.987 | 1.00 | 189.63 | C |
| ATOM | 865 | O | THR | A | 135 | −55.156 | −26.421 | 63.293 | 1.00 | 189.57 | O |
| ATOM | 866 | CB | THR | A | 135 | −57.817 | −25.865 | 61.411 | 1.00 | 190.26 | C |
| ATOM | 867 | OG1 | THR | A | 135 | −58.768 | −24.799 | 61.332 | 1.00 | 190.36 | O |
| ATOM | 868 | CG2 | THR | A | 135 | −58.394 | −27.072 | 60.721 | 1.00 | 190.30 | C |
| ATOM | 869 | N | TYR | A | 136 | −56.273 | −28.291 | 62.741 | 1.00 | 188.89 | N |
| ATOM | 870 | CA | TYR | A | 136 | −55.068 | −29.140 | 62.793 | 1.00 | 188.33 | C |
| ATOM | 871 | C | TYR | A | 136 | −54.935 | −30.163 | 61.655 | 1.00 | 187.81 | C |
| ATOM | 872 | O | TYR | A | 136 | −55.834 | −30.966 | 61.406 | 1.00 | 187.52 | O |
| ATOM | 873 | CB | TYR | A | 136 | −54.874 | −29.769 | 64.187 | 1.00 | 188.42 | C |
| ATOM | 874 | CG | TYR | A | 136 | −54.060 | −28.887 | 65.130 | 1.00 | 188.69 | C |
| ATOM | 875 | CD1 | TYR | A | 136 | −54.680 | −27.940 | 65.964 | 1.00 | 187.99 | C |
| ATOM | 876 | CD2 | TYR | A | 136 | −52.660 | −28.987 | 65.171 | 1.00 | 189.36 | C |
| ATOM | 877 | CE1 | TYR | A | 136 | −53.923 | −27.120 | 66.814 | 1.00 | 187.83 | C |
| ATOM | 878 | CE2 | TYR | A | 136 | −51.897 | −28.173 | 66.020 | 1.00 | 189.02 | C |
| ATOM | 879 | CZ | TYR | A | 136 | −52.533 | −27.247 | 66.835 | 1.00 | 188.39 | C |
| ATOM | 880 | OH | TYR | A | 136 | −51.763 | −26.462 | 67.661 | 1.00 | 188.13 | O |
| ATOM | 881 | N | VAL | A | 137 | −53.789 | −30.096 | 60.975 | 1.00 | 187.45 | N |
| ATOM | 882 | CA | VAL | A | 137 | −53.519 | −30.842 | 59.742 | 1.00 | 187.07 | C |
| ATOM | 883 | C | VAL | A | 137 | −52.690 | −32.088 | 60.058 | 1.00 | 186.77 | C |
| ATOM | 884 | O | VAL | A | 137 | −51.480 | −31.989 | 60.273 | 1.00 | 186.98 | O |
| ATOM | 885 | CB | VAL | A | 137 | −52.822 | −29.937 | 58.621 | 1.00 | 187.18 | C |
| ATOM | 886 | CG1 | VAL | A | 137 | −53.671 | −28.721 | 58.269 | 1.00 | 187.11 | C |
| ATOM | 887 | CG2 | VAL | A | 137 | −51.410 | −29.465 | 59.006 | 1.00 | 187.10 | C |
| ATOM | 888 | N | TRP | A | 138 | −53.338 | −33.253 | 60.098 | 1.00 | 186.28 | N |
| ATOM | 889 | CA | TRP | A | 138 | −52.665 | −34.504 | 60.506 | 1.00 | 185.84 | C |
| ATOM | 890 | C | TRP | A | 138 | −52.686 | −35.640 | 59.474 | 1.00 | 185.75 | C |
| ATOM | 891 | O | TRP | A | 138 | −53.748 | −36.016 | 58.961 | 1.00 | 185.68 | O |
| ATOM | 892 | CB | TRP | A | 138 | −53.256 | −35.012 | 61.814 | 1.00 | 185.56 | C |
| ATOM | 893 | CG | TRP | A | 138 | −52.703 | −34.389 | 63.057 | 1.00 | 185.26 | C |
| ATOM | 894 | CD1 | TRP | A | 138 | −52.040 | −33.194 | 63.178 | 1.00 | 185.04 | C |
| ATOM | 895 | CD2 | TRP | A | 138 | −52.813 | −34.920 | 64.375 | 1.00 | 184.94 | C |
| ATOM | 896 | NE1 | TRP | A | 138 | −51.713 | −32.963 | 64.493 | 1.00 | 184.86 | N |
| ATOM | 897 | CE2 | TRP | A | 138 | −52.179 | −34.009 | 65.249 | 1.00 | 185.03 | C |
| ATOM | 898 | CE3 | TRP | A | 138 | −53.386 | −36.083 | 64.905 | 1.00 | 184.38 | C |
| ATOM | 899 | CZ2 | TRP | A | 138 | −52.103 | −34.229 | 66.621 | 1.00 | 185.26 | C |
| ATOM | 900 | CZ3 | TRP | A | 138 | −53.301 | −36.304 | 66.261 | 1.00 | 184.67 | C |
| ATOM | 901 | CH2 | TRP | A | 138 | −52.667 | −35.383 | 67.107 | 1.00 | 184.98 | C |
| ATOM | 902 | N | GLN | A | 139 | −51.508 | −36.202 | 59.206 | 1.00 | 185.59 | N |
| ATOM | 903 | CA | GLN | A | 139 | −51.360 | −37.225 | 58.184 | 1.00 | 185.61 | C |
| ATOM | 904 | C | GLN | A | 139 | −50.688 | −38.468 | 58.696 | 1.00 | 185.51 | C |
| ATOM | 905 | O | GLN | A | 139 | −49.707 | −38.401 | 59.437 | 1.00 | 185.45 | O |
| ATOM | 906 | CB | GLN | A | 139 | −50.495 | −36.706 | 57.052 | 1.00 | 185.76 | C |
| ATOM | 907 | CG | GLN | A | 139 | −51.179 | −35.797 | 56.077 | 1.00 | 186.88 | C |
| ATOM | 908 | CD | GLN | A | 139 | −50.197 | −34.826 | 55.460 | 1.00 | 188.62 | C |
| ATOM | 909 | OE1 | GLN | A | 139 | −49.451 | −34.146 | 56.177 | 1.00 | 188.99 | O |
| ATOM | 910 | NE2 | GLN | A | 139 | −50.181 | −34.756 | 54.126 | 1.00 | 189.64 | N |
| ATOM | 911 | N | VAL | A | 140 | −51.212 | −39.609 | 58.277 | 1.00 | 185.55 | N |
| ATOM | 912 | CA | VAL | A | 140 | −50.463 | −40.844 | 58.355 | 1.00 | 185.78 | C |
| ATOM | 913 | C | VAL | A | 140 | −50.014 | −41.195 | 56.942 | 1.00 | 186.41 | C |
| ATOM | 914 | O | VAL | A | 140 | −50.840 | −41.335 | 56.047 | 1.00 | 186.58 | O |
| ATOM | 915 | CB | VAL | A | 140 | −51.271 | −42.000 | 59.001 | 1.00 | 185.52 | C |
| ATOM | 916 | CG1 | VAL | A | 140 | −51.163 | −41.935 | 60.500 | 1.00 | 185.06 | C |
| ATOM | 917 | CG2 | VAL | A | 140 | −52.723 | −41.962 | 58.584 | 1.00 | 184.99 | C |
| ATOM | 918 | N | LEU | A | 141 | −48.706 | −41.278 | 56.723 | 1.00 | 187.09 | N |
| ATOM | 919 | CA | LEU | A | 141 | −48.184 | −41.795 | 55.460 | 1.00 | 187.88 | C |
| ATOM | 920 | C | LEU | A | 141 | −47.930 | −43.290 | 55.634 | 1.00 | 188.52 | C |
| ATOM | 921 | O | LEU | A | 141 | −48.196 | −43.846 | 56.705 | 1.00 | 188.60 | O |
| ATOM | 922 | CB | LEU | A | 141 | −46.888 | −41.092 | 55.056 | 1.00 | 187.77 | C |
| ATOM | 923 | CG | LEU | A | 141 | −46.601 | −39.696 | 55.599 | 1.00 | 187.86 | C |
| ATOM | 924 | CD1 | LEU | A | 141 | −45.100 | −39.407 | 55.492 | 1.00 | 188.25 | C |
| ATOM | 925 | CD2 | LEU | A | 141 | −47.449 | −38.621 | 54.909 | 1.00 | 187.30 | C |
| ATOM | 926 | N | LYS | A | 142 | −47.420 | −43.937 | 54.584 | 1.00 | 189.26 | N |
| ATOM | 927 | CA | LYS | A | 142 | −47.018 | −45.342 | 54.658 | 1.00 | 189.92 | C |
| ATOM | 928 | C | LYS | A | 142 | −45.884 | −45.503 | 55.677 | 1.00 | 190.61 | C |
| ATOM | 929 | O | LYS | A | 142 | −45.889 | −46.430 | 56.499 | 1.00 | 190.52 | O |
| ATOM | 930 | CB | LYS | A | 142 | −46.577 | −45.835 | 53.278 | 1.00 | 189.71 | C |
| ATOM | 931 | CG | LYS | A | 142 | −46.784 | −47.327 | 53.047 | 1.00 | 189.85 | C |
| ATOM | 932 | CD | LYS | A | 142 | −48.273 | −47.711 | 53.036 | 1.00 | 189.89 | C |
| ATOM | 933 | CE | LYS | A | 142 | −48.502 | −49.222 | 52.900 | 1.00 | 189.76 | C |
| ATOM | 934 | NZ | LYS | A | 142 | −48.291 | −49.731 | 51.512 | 1.00 | 189.12 | N |
| ATOM | 935 | N | GLU | A | 143 | −44.948 | −44.549 | 55.620 | 1.00 | 191.56 | N |
| ATOM | 936 | CA | GLU | A | 143 | −43.726 | −44.484 | 56.444 | 1.00 | 192.27 | C |
| ATOM | 937 | C | GLU | A | 143 | −43.968 | −44.353 | 57.957 | 1.00 | 192.08 | C |
| ATOM | 938 | O | GLU | A | 143 | −43.023 | −44.498 | 58.732 | 1.00 | 192.34 | O |
| ATOM | 939 | CB | GLU | A | 143 | −42.805 | −43.333 | 55.962 | 1.00 | 192.48 | C |
| ATOM | 940 | CG | GLU | A | 143 | −42.263 | −43.457 | 54.496 | 1.00 | 193.14 | C |
| ATOM | 941 | CD | GLU | A | 143 | −41.947 | −42.095 | 53.810 | 1.00 | 193.05 | C |
| ATOM | 942 | OE1 | GLU | A | 143 | −42.548 | −41.045 | 54.171 | 1.00 | 194.08 | O |
| ATOM | 943 | OE2 | GLU | A | 143 | −41.097 | −42.087 | 52.886 | 1.00 | 192.94 | O |
| ATOM | 944 | N | ASN | A | 144 | −45.204 | −44.068 | 58.377 | 1.00 | 191.88 | N |
| ATOM | 945 | CA | ASN | A | 144 | −45.544 | −44.051 | 59.816 | 1.00 | 191.57 | C |
| ATOM | 946 | C | ASN | A | 144 | −46.716 | −44.939 | 60.223 | 1.00 | 191.34 | C |
| ATOM | 947 | O | ASN | A | 144 | −47.231 | −44.843 | 61.336 | 1.00 | 191.03 | O |
| ATOM | 948 | CB | ASN | A | 144 | −45.689 | −42.616 | 60.384 | 1.00 | 191.64 | C |
| ATOM | 949 | CG | ASN | A | 144 | −46.654 | −41.737 | 59.595 | 1.00 | 191.16 | C |
| ATOM | 950 | OD1 | ASN | A | 144 | −46.499 | −40.516 | 59.564 | 1.00 | 190.32 | O |
| ATOM | 951 | ND2 | ASN | A | 144 | −47.658 | −42.344 | 58.982 | 1.00 | 191.03 | N |
| ATOM | 952 | N | GLY | A | 145 | −47.124 | −45.806 | 59.305 | 1.00 | 191.27 | N |
| ATOM | 953 | CA | GLY | A | 145 | −48.020 | −46.901 | 59.631 | 1.00 | 191.36 | C |
| ATOM | 954 | C | GLY | A | 145 | −47.180 | −47.979 | 60.286 | 1.00 | 191.45 | C |
| ATOM | 955 | O | GLY | A | 145 | −45.957 | −47.867 | 60.301 | 1.00 | 191.62 | O |
| ATOM | 956 | N | PRO | A | 146 | −47.818 | −49.043 | 60.802 | 1.00 | 191.46 | N |
| ATOM | 957 | CA | PRO | A | 146 | −47.149 | −49.999 | 61.662 | 1.00 | 191.70 | C |
| ATOM | 958 | C | PRO | A | 146 | −46.494 | −51.097 | 60.860 | 1.00 | 192.30 | C |
| ATOM | 959 | O | PRO | A | 146 | −47.038 | −51.538 | 59.842 | 1.00 | 192.20 | O |
| ATOM | 960 | CB | PRO | A | 146 | −48.294 | −50.581 | 62.458 | 1.00 | 191.54 | C |
| ATOM | 961 | CG | PRO | A | 146 | −49.404 | −50.616 | 61.475 | 1.00 | 191.46 | C |
| ATOM | 962 | CD | PRO | A | 146 | −49.219 | −49.427 | 60.570 | 1.00 | 191.43 | C |
| ATOM | 963 | N | MET | A | 147 | −45.345 | −51.551 | 61.351 | 1.00 | 193.26 | N |
| ATOM | 964 | CA | MET | A | 147 | −44.470 | −52.457 | 60.599 | 1.00 | 194.13 | C |
| ATOM | 965 | C | MET | A | 147 | −45.037 | −53.835 | 60.323 | 1.00 | 194.28 | C |
| ATOM | 966 | O | MET | A | 147 | −45.782 | −54.397 | 61.128 | 1.00 | 194.15 | O |
| ATOM | 967 | CB | MET | A | 147 | −43.085 | −52.576 | 61.248 | 1.00 | 194.33 | C |
| ATOM | 968 | CG | MET | A | 147 | −42.106 | −51.476 | 60.820 | 1.00 | 195.53 | C |
| ATOM | 969 | SD | MET | A | 147 | −41.887 | −51.236 | 59.029 | 1.00 | 197.22 | S |
| ATOM | 970 | CE | MET | A | 147 | −40.727 | −52.557 | 58.606 | 1.00 | 197.42 | C |
| ATOM | 971 | N | ALA | A | 148 | −44.648 | −54.364 | 59.167 | 1.00 | 194.68 | N |
| ATOM | 972 | CA | ALA | A | 148 | −45.079 | −55.667 | 58.710 | 1.00 | 195.06 | C |
| ATOM | 973 | C | ALA | A | 148 | −45.593 | −56.534 | 59.873 | 1.00 | 195.20 | C |
| ATOM | 974 | O | ALA | A | 148 | −46.786 | −56.818 | 59.934 | 1.00 | 195.49 | O |
| ATOM | 975 | CB | ALA | A | 148 | −43.955 | −56.364 | 57.902 | 1.00 | 195.05 | C |
| ATOM | 976 | N | SER | A | 149 | −44.732 | −56.908 | 60.816 | 1.00 | 195.15 | N |
| ATOM | 977 | CA | SER | A | 149 | −45.173 | −57.798 | 61.878 | 1.00 | 195.21 | C |
| ATOM | 978 | C | SER | A | 149 | −44.924 | −57.249 | 63.270 | 1.00 | 195.61 | C |
| ATOM | 979 | O | SER | A | 149 | −43.808 | −57.275 | 63.777 | 1.00 | 195.71 | O |
| ATOM | 980 | CB | SER | A | 149 | −44.551 | −59.177 | 61.716 | 1.00 | 194.97 | C |
| ATOM | 981 | OG | SER | A | 149 | −43.147 | −59.078 | 61.723 | 1.00 | 194.79 | O |
| ATOM | 982 | N | ASP | A | 150 | −45.996 | −56.748 | 63.868 | 1.00 | 196.13 | N |
| ATOM | 983 | CA | ASP | A | 150 | −46.004 | −56.206 | 65.216 | 1.00 | 196.70 | C |
| ATOM | 984 | C | ASP | A | 150 | −47.476 | −55.894 | 65.530 | 1.00 | 197.30 | C |
| ATOM | 985 | O | ASP | A | 150 | −48.332 | −56.169 | 64.677 | 1.00 | 197.26 | O |
| ATOM | 986 | CB | ASP | A | 150 | −45.118 | −54.947 | 65.297 | 1.00 | 196.69 | C |
| ATOM | 987 | CG | ASP | A | 150 | −45.774 | −53.698 | 64.691 | 1.00 | 196.44 | C |
| ATOM | 988 | OD1 | ASP | A | 150 | −46.642 | −53.849 | 63.809 | 1.00 | 196.71 | O |
| ATOM | 989 | OD2 | ASP | A | 150 | −45.411 | −52.563 | 65.095 | 1.00 | 195.40 | O |
| ATOM | 990 | N | PRO | A | 151 | −47.782 | −55.343 | 66.743 | 1.00 | 197.85 | N |
| ATOM | 991 | CA | PRO | A | 151 | −49.097 | −54.782 | 67.079 | 1.00 | 197.79 | C |
| ATOM | 992 | C | PRO | A | 151 | −49.969 | −54.377 | 65.888 | 1.00 | 197.59 | C |
| ATOM | 993 | O | PRO | A | 151 | −49.604 | −53.485 | 65.116 | 1.00 | 197.32 | O |
| ATOM | 994 | CB | PRO | A | 151 | −48.723 | −53.556 | 67.929 | 1.00 | 197.87 | C |
| ATOM | 995 | CG | PRO | A | 151 | −47.444 | −53.991 | 68.676 | 1.00 | 198.13 | C |
| ATOM | 996 | CD | PRO | A | 151 | −46.900 | −55.242 | 67.929 | 1.00 | 198.22 | C |
| ATOM | 997 | N | LEU | A | 152 | −51.114 | −55.053 | 65.762 | 1.00 | 197.54 | N |
| ATOM | 998 | CA | LEU | A | 152 | −52.093 | −54.792 | 64.708 | 1.00 | 197.47 | C |
| ATOM | 999 | C | LEU | A | 152 | −52.307 | −53.312 | 64.629 | 1.00 | 197.38 | C |
| ATOM | 1000 | O | LEU | A | 152 | −52.569 | −52.759 | 63.567 | 1.00 | 197.07 | O |
| ATOM | 1001 | CB | LEU | A | 152 | −53.432 | −55.465 | 65.048 | 1.00 | 197.50 | C |
| ATOM | 1002 | CG | LEU | A | 152 | −54.662 | −55.338 | 64.124 | 1.00 | 197.32 | C |
| ATOM | 1003 | CD1 | LEU | A | 152 | −55.519 | −54.095 | 64.398 | 1.00 | 196.80 | C |
| ATOM | 1004 | CD2 | LEU | A | 152 | −54.290 | −55.453 | 62.638 | 1.00 | 196.95 | C |
| ATOM | 1005 | N | CYS | A | 153 | −52.182 | −52.688 | 65.789 | 1.00 | 197.64 | N |
| ATOM | 1006 | CA | CYS | A | 153 | −52.544 | −51.319 | 65.944 | 1.00 | 198.13 | C |
| ATOM | 1007 | C | CYS | A | 153 | −51.739 | −50.573 | 66.992 | 1.00 | 198.34 | C |
| ATOM | 1008 | O | CYS | A | 153 | −51.766 | −50.900 | 68.180 | 1.00 | 198.34 | O |
| ATOM | 1009 | CB | CYS | A | 153 | −54.008 | −51.229 | 66.272 | 1.00 | 198.10 | C |
| ATOM | 1010 | SG | CYS | A | 153 | −54.467 | −49.578 | 66.027 | 1.00 | 199.51 | S |
| ATOM | 1011 | N | LEU | A | 154 | −51.052 | −49.535 | 66.544 | 1.00 | 198.75 | N |
| ATOM | 1012 | CA | LEU | A | 154 | −50.054 | −48.911 | 67.376 | 1.00 | 199.33 | C |
| ATOM | 1013 | C | LEU | A | 154 | −50.558 | −47.971 | 68.443 | 1.00 | 199.57 | C |
| ATOM | 1014 | O | LEU | A | 154 | −51.473 | −47.169 | 68.234 | 1.00 | 199.73 | O |
| ATOM | 1015 | CB | LEU | A | 154 | −48.983 | −48.232 | 66.539 | 1.00 | 199.47 | C |
| ATOM | 1016 | CG | LEU | A | 154 | −47.640 | −48.931 | 66.744 | 1.00 | 200.42 | C |
| ATOM | 1017 | CD1 | LEU | A | 154 | −46.673 | −48.594 | 65.589 | 1.00 | 200.85 | C |
| ATOM | 1018 | CD2 | LEU | A | 154 | −47.035 | −48.638 | 68.164 | 1.00 | 200.76 | C |
| ATOM | 1019 | N | THR | A | 155 | −49.910 | −48.099 | 69.592 | 1.00 | 199.80 | N |
| ATOM | 1020 | CA | THR | A | 155 | −50.087 | −47.238 | 70.746 | 1.00 | 199.95 | C |
| ATOM | 1021 | C | THR | A | 155 | −49.458 | −45.878 | 70.454 | 1.00 | 199.31 | C |
| ATOM | 1022 | O | THR | A | 155 | −48.238 | −45.805 | 70.272 | 1.00 | 199.34 | O |
| ATOM | 1023 | CB | THR | A | 155 | −49.286 | −47.822 | 71.933 | 1.00 | 200.35 | C |
| ATOM | 1024 | OG1 | THR | A | 155 | −48.562 | −48.988 | 71.496 | 1.00 | 201.51 | O |
| ATOM | 1025 | CG2 | THR | A | 155 | −50.185 | −48.134 | 73.136 | 1.00 | 200.31 | C |
| ATOM | 1026 | N | TYR | A | 156 | −50.271 | −44.823 | 70.388 | 1.00 | 198.46 | N |
| ATOM | 1027 | CA | TYR | A | 156 | −49.754 | −43.453 | 70.378 | 1.00 | 197.73 | C |
| ATOM | 1028 | C | TYR | A | 156 | −50.708 | −42.522 | 71.090 | 1.00 | 197.07 | C |
| ATOM | 1029 | O | TYR | A | 156 | −51.901 | −42.753 | 71.105 | 1.00 | 196.98 | O |
| ATOM | 1030 | CB | TYR | A | 156 | −49.470 | −42.937 | 68.963 | 1.00 | 197.99 | C |
| ATOM | 1031 | CG | TYR | A | 156 | −48.333 | −43.637 | 68.220 | 1.00 | 198.45 | C |
| ATOM | 1032 | CD1 | TYR | A | 156 | −48.474 | −43.994 | 66.877 | 1.00 | 198.72 | C |
| ATOM | 1033 | CD2 | TYR | A | 156 | −47.117 | −43.932 | 68.849 | 1.00 | 198.81 | C |
| ATOM | 1034 | CE1 | TYR | A | 156 | −47.448 | −44.631 | 66.180 | 1.00 | 198.38 | C |
| ATOM | 1035 | CE2 | TYR | A | 156 | −46.089 | −44.583 | 68.164 | 1.00 | 198.72 | C |
| ATOM | 1036 | CZ | TYR | A | 156 | −46.264 | −44.921 | 66.828 | 1.00 | 198.47 | C |
| ATOM | 1037 | OH | TYR | A | 156 | −45.261 | −45.557 | 66.137 | 1.00 | 198.43 | O |
| ATOM | 1038 | N | SER | A | 157 | −50.175 | −41.473 | 71.694 | 1.00 | 196.47 | N |
| ATOM | 1039 | CA | SER | A | 157 | −51.000 | −40.522 | 72.431 | 1.00 | 196.12 | C |
| ATOM | 1040 | C | SER | A | 157 | −50.944 | −39.158 | 71.760 | 1.00 | 195.97 | C |
| ATOM | 1041 | O | SER | A | 157 | −50.099 | −38.952 | 70.884 | 1.00 | 196.33 | O |
| ATOM | 1042 | CB | SER | A | 157 | −50.522 | −40.418 | 73.883 | 1.00 | 196.13 | C |
| ATOM | 1043 | OG | SER | A | 157 | −49.192 | −39.939 | 73.967 | 1.00 | 195.30 | O |
| ATOM | 1044 | N | TYR | A | 158 | −51.836 | −38.234 | 72.143 | 1.00 | 195.38 | N |
| ATOM | 1045 | CA | TYR | A | 158 | −51.720 | −36.840 | 71.679 | 1.00 | 194.59 | C |
| ATOM | 1046 | C | TYR | A | 158 | −51.604 | −35.832 | 72.793 | 1.00 | 193.93 | C |
| ATOM | 1047 | O | TYR | A | 158 | −52.509 | −35.650 | 73.596 | 1.00 | 193.77 | O |
| ATOM | 1048 | CB | TYR | A | 158 | −52.793 | −36.440 | 70.665 | 1.00 | 194.84 | C |
| ATOM | 1049 | CG | TYR | A | 158 | −54.239 | −36.531 | 71.096 | 1.00 | 195.03 | C |
| ATOM | 1050 | CD1 | TYR | A | 158 | −54.799 | −35.589 | 71.956 | 1.00 | 195.41 | C |
| ATOM | 1051 | CD2 | TYR | A | 158 | −55.072 | −37.521 | 70.576 | 1.00 | 195.45 | C |
| ATOM | 1052 | CE1 | TYR | A | 158 | −56.142 | −35.657 | 72.324 | 1.00 | 195.90 | C |
| ATOM | 1053 | CE2 | TYR | A | 158 | −56.416 | −37.597 | 70.927 | 1.00 | 195.79 | C |
| ATOM | 1054 | CZ | TYR | A | 158 | −56.946 | −36.664 | 71.802 | 1.00 | 195.94 | C |
| ATOM | 1055 | OH | TYR | A | 158 | −58.277 | −36.740 | 72.148 | 1.00 | 195.89 | O |
| ATOM | 1056 | N | LEU | A | 159 | −50.467 | −35.166 | 72.809 | 1.00 | 193.25 | N |
| ATOM | 1057 | CA | LEU | A | 159 | −50.077 | −34.358 | 73.925 | 1.00 | 192.98 | C |
| ATOM | 1058 | C | LEU | A | 159 | −50.577 | −32.940 | 73.766 | 1.00 | 193.13 | C |
| ATOM | 1059 | O | LEU | A | 159 | −51.369 | −32.656 | 72.877 | 1.00 | 193.16 | O |
| ATOM | 1060 | CB | LEU | A | 159 | −48.562 | −34.382 | 74.007 | 1.00 | 192.71 | C |
| ATOM | 1061 | CG | LEU | A | 159 | −47.817 | −33.731 | 75.164 | 1.00 | 192.76 | C |
| ATOM | 1062 | CD1 | LEU | A | 159 | −48.537 | −33.891 | 76.501 | 1.00 | 193.50 | C |
| ATOM | 1063 | CD2 | LEU | A | 159 | −46.440 | −34.341 | 75.223 | 1.00 | 192.78 | C |
| ATOM | 1064 | N | SER | A | 160 | −50.131 | −32.067 | 74.663 | 1.00 | 193.47 | N |
| ATOM | 1065 | CA | SER | A | 160 | −50.232 | −30.626 | 74.503 | 1.00 | 193.85 | C |
| ATOM | 1066 | C | SER | A | 160 | −48.996 | −29.975 | 75.108 | 1.00 | 194.24 | C |
| ATOM | 1067 | O | SER | A | 160 | −48.738 | −30.145 | 76.302 | 1.00 | 194.08 | O |
| ATOM | 1068 | CB | SER | A | 160 | −51.466 | −30.092 | 75.204 | 1.00 | 193.71 | C |
| ATOM | 1069 | OG | SER | A | 160 | −51.421 | −28.681 | 75.201 | 1.00 | 193.74 | O |
| ATOM | 1070 | N | HIS | A | 161 | −48.230 | −29.244 | 74.291 | 1.00 | 194.88 | N |
| ATOM | 1071 | CA | HIS | A | 161 | −47.019 | −28.565 | 74.800 | 1.00 | 195.43 | C |
| ATOM | 1072 | C | HIS | A | 161 | −46.749 | −27.106 | 74.354 | 1.00 | 195.76 | C |
| ATOM | 1073 | O | HIS | A | 161 | −46.411 | −26.795 | 73.203 | 1.00 | 195.83 | O |
| ATOM | 1074 | CB | HIS | A | 161 | −45.780 | −29.523 | 74.852 | 1.00 | 195.41 | C |
| ATOM | 1075 | CG | HIS | A | 161 | −44.698 | −29.267 | 73.836 | 1.00 | 195.17 | C |
| ATOM | 1076 | ND1 | HIS | A | 161 | −44.483 | −30.097 | 72.757 | 1.00 | 194.24 | N |
| ATOM | 1077 | CD2 | HIS | A | 161 | −43.709 | −28.339 | 73.797 | 1.00 | 195.07 | C |
| ATOM | 1078 | CE1 | HIS | A | 161 | −43.438 | −29.666 | 72.073 | 1.00 | 194.14 | C |
| ATOM | 1079 | NE2 | HIS | A | 161 | −42.953 | −28.596 | 72.680 | 1.00 | 194.40 | N |
| ATOM | 1080 | N | VAL | A | 162 | −46.976 | −26.226 | 75.321 | 1.00 | 196.05 | N |
| ATOM | 1081 | CA | VAL | A | 162 | −46.566 | −24.841 | 75.297 | 1.00 | 196.63 | C |
| ATOM | 1082 | C | VAL | A | 162 | −45.523 | −24.735 | 76.412 | 1.00 | 197.02 | C |
| ATOM | 1083 | O | VAL | A | 162 | −44.356 | −24.414 | 76.150 | 1.00 | 197.23 | O |
| ATOM | 1084 | CB | VAL | A | 162 | −47.775 | −23.929 | 75.589 | 1.00 | 196.65 | C |
| ATOM | 1085 | CG1 | VAL | A | 162 | −47.342 | −22.494 | 75.967 | 1.00 | 196.90 | C |
| ATOM | 1086 | CG2 | VAL | A | 162 | −48.729 | −23.945 | 74.412 | 1.00 | 196.66 | C |
| ATOM | 1087 | N | ASP | A | 163 | −45.979 | −24.995 | 77.649 | 1.00 | 197.42 | N |
| ATOM | 1088 | CA | ASP | A | 163 | −45.146 | −25.280 | 78.842 | 1.00 | 197.41 | C |
| ATOM | 1089 | C | ASP | A | 163 | −45.382 | −26.760 | 79.141 | 1.00 | 197.12 | C |
| ATOM | 1090 | O | ASP | A | 163 | −46.357 | −27.127 | 79.805 | 1.00 | 196.84 | O |
| ATOM | 1091 | CB | ASP | A | 163 | −45.538 | −24.382 | 80.054 | 1.00 | 197.63 | C |
| ATOM | 1092 | CG | ASP | A | 163 | −44.735 | −24.689 | 81.365 | 1.00 | 197.62 | C |
| ATOM | 1093 | OD1 | ASP | A | 163 | −45.145 | −24.172 | 82.431 | 1.00 | 198.16 | O |
| ATOM | 1094 | OD2 | ASP | A | 163 | −43.712 | −25.416 | 81.359 | 1.00 | 197.41 | O |
| ATOM | 1095 | N | LEU | A | 164 | −44.494 | −27.600 | 78.619 | 1.00 | 196.96 | N |
| ATOM | 1096 | CA | LEU | A | 164 | −44.665 | −29.038 | 78.695 | 1.00 | 196.96 | C |
| ATOM | 1097 | C | LEU | A | 164 | −45.081 | −29.503 | 80.082 | 1.00 | 197.11 | C |
| ATOM | 1098 | O | LEU | A | 164 | −46.061 | −30.236 | 80.206 | 1.00 | 197.59 | O |
| ATOM | 1099 | CB | LEU | A | 164 | −43.402 | −29.770 | 78.263 | 1.00 | 196.83 | C |
| ATOM | 1100 | CG | LEU | A | 164 | −43.688 | −31.162 | 77.702 | 1.00 | 196.49 | C |
| ATOM | 1101 | CD1 | LEU | A | 164 | −42.582 | −31.511 | 76.725 | 1.00 | 197.13 | C |
| ATOM | 1102 | CD2 | LEU | A | 164 | −43.865 | −32.252 | 78.780 | 1.00 | 195.22 | C |
| ATOM | 1103 | N | VAL | A | 165 | −44.345 | −29.076 | 81.113 | 1.00 | 196.92 | N |
| ATOM | 1104 | CA | VAL | A | 165 | −44.621 | −29.475 | 82.506 | 1.00 | 196.55 | C |
| ATOM | 1105 | C | VAL | A | 165 | −46.054 | −29.114 | 82.937 | 1.00 | 196.34 | C |
| ATOM | 1106 | O | VAL | A | 165 | −46.778 | −29.958 | 83.488 | 1.00 | 196.27 | O |
| ATOM | 1107 | CB | VAL | A | 165 | −43.595 | −28.862 | 83.504 | 1.00 | 196.55 | C |
| ATOM | 1108 | CG1 | VAL | A | 165 | −43.762 | −29.466 | 84.892 | 1.00 | 196.37 | C |
| ATOM | 1109 | CG2 | VAL | A | 165 | −42.171 | −29.067 | 83.013 | 1.00 | 196.53 | C |
| ATOM | 1110 | N | LYS | A | 166 | −46.446 | −27.867 | 82.656 | 1.00 | 195.99 | N |
| ATOM | 1111 | CA | LYS | A | 166 | −47.741 | −27.302 | 83.060 | 1.00 | 195.49 | C |
| ATOM | 1112 | C | LYS | A | 166 | −48.888 | −27.963 | 82.292 | 1.00 | 195.17 | C |
| ATOM | 1113 | O | LYS | A | 166 | −49.786 | −28.553 | 82.893 | 1.00 | 195.17 | O |
| ATOM | 1114 | CB | LYS | A | 166 | −47.741 | −25.781 | 82.841 | 1.00 | 195.45 | C |
| ATOM | 1115 | CG | LYS | A | 166 | −48.568 | −24.958 | 83.809 | 1.00 | 194.72 | C |
| ATOM | 1116 | CD | LYS | A | 166 | −48.317 | −23.498 | 83.537 | 1.00 | 193.71 | C |
| ATOM | 1117 | CE | LYS | A | 166 | −49.529 | −22.675 | 83.854 | 1.00 | 193.61 | C |
| ATOM | 1118 | NZ | LYS | A | 166 | −49.718 | −21.679 | 82.771 | 1.00 | 193.60 | N |
| ATOM | 1119 | N | ASP | A | 167 | −48.827 | −27.881 | 80.964 | 1.00 | 194.68 | N |
| ATOM | 1120 | CA | ASP | A | 167 | −49.856 | −28.430 | 80.080 | 1.00 | 194.30 | C |
| ATOM | 1121 | C | ASP | A | 167 | −50.233 | −29.863 | 80.422 | 1.00 | 193.64 | C |
| ATOM | 1122 | O | ASP | A | 167 | −51.355 | −30.302 | 80.167 | 1.00 | 193.39 | O |
| ATOM | 1123 | CB | ASP | A | 167 | −49.373 | −28.386 | 78.629 | 1.00 | 194.65 | C |
| ATOM | 1124 | CG | ASP | A | 167 | −49.055 | −26.977 | 78.151 | 1.00 | 195.49 | C |
| ATOM | 1125 | OD1 | ASP | A | 167 | −49.238 | −26.008 | 78.932 | 1.00 | 196.43 | O |
| ATOM | 1126 | OD2 | ASP | A | 167 | −48.623 | −26.847 | 76.981 | 1.00 | 196.05 | O |
| ATOM | 1127 | N | LEU | A | 168 | −49.281 | −30.588 | 80.993 | 1.00 | 193.00 | N |
| ATOM | 1128 | CA | LEU | A | 168 | −49.479 | −31.985 | 81.298 | 1.00 | 192.48 | C |
| ATOM | 1129 | C | LEU | A | 168 | −50.074 | −32.141 | 82.688 | 1.00 | 192.28 | C |
| ATOM | 1130 | O | LEU | A | 168 | −51.266 | −32.420 | 82.817 | 1.00 | 192.33 | O |
| ATOM | 1131 | CB | LEU | A | 168 | −48.171 | −32.760 | 81.163 | 1.00 | 192.33 | C |
| ATOM | 1132 | CG | LEU | A | 168 | −48.309 | −34.195 | 80.670 | 1.00 | 191.81 | C |
| ATOM | 1133 | CD1 | LEU | A | 168 | −47.062 | −34.556 | 79.895 | 1.00 | 191.86 | C |
| ATOM | 1134 | CD2 | LEU | A | 168 | −48.534 | −35.151 | 81.832 | 1.00 | 191.44 | C |
| ATOM | 1135 | N | ASN | A | 169 | −49.261 | −31.944 | 83.724 | 1.00 | 191.86 | N |
| ATOM | 1136 | CA | ASN | A | 169 | −49.725 | −32.157 | 85.097 | 1.00 | 191.42 | C |
| ATOM | 1137 | C | ASN | A | 169 | −51.119 | −31.608 | 85.315 | 1.00 | 191.04 | C |
| ATOM | 1138 | O | ASN | A | 169 | −51.940 | −32.238 | 85.969 | 1.00 | 191.00 | O |
| ATOM | 1139 | CB | ASN | A | 169 | −48.742 | −31.588 | 86.116 | 1.00 | 191.42 | C |
| ATOM | 1140 | CG | ASN | A | 169 | −47.546 | −32.492 | 86.329 | 1.00 | 191.73 | C |
| ATOM | 1141 | OD1 | ASN | A | 169 | −47.129 | −32.738 | 87.464 | 1.00 | 191.74 | O |
| ATOM | 1142 | ND2 | ASN | A | 169 | −46.996 | −33.010 | 85.231 | 1.00 | 192.14 | N |
| ATOM | 1143 | N | SER | A | 170 | −51.380 | −30.445 | 84.729 | 1.00 | 190.71 | N |
| ATOM | 1144 | CA | SER | A | 170 | −52.711 | −29.864 | 84.686 | 1.00 | 190.41 | C |
| ATOM | 1145 | C | SER | A | 170 | −53.764 | −30.924 | 84.304 | 1.00 | 190.20 | C |
| ATOM | 1146 | O | SER | A | 170 | −54.677 | −31.184 | 85.086 | 1.00 | 190.28 | O |
| ATOM | 1147 | CB | SER | A | 170 | −52.728 | −28.684 | 83.712 | 1.00 | 190.42 | C |
| ATOM | 1148 | OG | SER | A | 170 | −53.147 | −27.492 | 84.350 | 1.00 | 190.16 | O |
| ATOM | 1149 | N | GLY | A | 171 | −53.627 | −31.540 | 83.125 | 1.00 | 189.84 | N |
| ATOM | 1150 | CA | GLY | A | 171 | −54.510 | −32.645 | 82.713 | 1.00 | 189.33 | C |
| ATOM | 1151 | C | GLY | A | 171 | −54.862 | −32.771 | 81.234 | 1.00 | 189.07 | C |
| ATOM | 1152 | O | GLY | A | 171 | −55.942 | −33.271 | 80.896 | 1.00 | 188.88 | O |
| ATOM | 1153 | N | LEU | A | 172 | −53.958 | −32.324 | 80.358 | 1.00 | 188.89 | N |
| ATOM | 1154 | CA | LEU | A | 172 | −54.131 | −32.444 | 78.902 | 1.00 | 188.72 | C |
| ATOM | 1155 | C | LEU | A | 172 | −53.401 | −33.658 | 78.329 | 1.00 | 188.74 | C |
| ATOM | 1156 | O | LEU | A | 172 | −52.176 | −33.643 | 78.170 | 1.00 | 188.94 | O |
| ATOM | 1157 | CB | LEU | A | 172 | −53.656 | −31.179 | 78.183 | 1.00 | 188.51 | C |
| ATOM | 1158 | CG | LEU | A | 172 | −54.499 | −29.922 | 78.371 | 1.00 | 188.53 | C |
| ATOM | 1159 | CD1 | LEU | A | 172 | −53.592 | −28.728 | 78.495 | 1.00 | 188.96 | C |
| ATOM | 1160 | CD2 | LEU | A | 172 | −55.504 | −29.721 | 77.244 | 1.00 | 188.20 | C |
| ATOM | 1161 | N | ILE | A | 173 | −54.165 | −34.709 | 78.033 | 1.00 | 188.64 | N |
| ATOM | 1162 | CA | ILE | A | 173 | −53.650 | −35.912 | 77.365 | 1.00 | 188.39 | C |
| ATOM | 1163 | C | ILE | A | 173 | −54.797 | −36.780 | 76.815 | 1.00 | 188.70 | C |
| ATOM | 1164 | O | ILE | A | 173 | −55.830 | −36.978 | 77.478 | 1.00 | 188.83 | O |
| ATOM | 1165 | CB | ILE | A | 173 | −52.653 | −36.732 | 78.259 | 1.00 | 188.00 | C |
| ATOM | 1166 | CG1 | ILE | A | 173 | −52.190 | −38.003 | 77.555 | 1.00 | 187.47 | C |
| ATOM | 1167 | CG2 | ILE | A | 173 | −53.277 | −37.115 | 79.575 | 1.00 | 187.78 | C |
| ATOM | 1168 | CD1 | ILE | A | 173 | −51.354 | −37.775 | 76.336 | 1.00 | 187.25 | C |
| ATOM | 1169 | N | GLY | A | 174 | −54.608 | −37.266 | 75.586 | 1.00 | 188.83 | N |
| ATOM | 1170 | CA | GLY | A | 174 | −55.546 | −38.186 | 74.940 | 1.00 | 188.82 | C |
| ATOM | 1171 | C | GLY | A | 174 | −54.844 | −39.327 | 74.223 | 1.00 | 188.71 | C |
| ATOM | 1172 | O | GLY | A | 174 | −53.632 | −39.284 | 74.007 | 1.00 | 188.44 | O |
| ATOM | 1173 | N | ALA | A | 175 | −55.612 | −40.352 | 73.865 | 1.00 | 188.76 | N |
| ATOM | 1174 | CA | ALA | A | 175 | −55.085 | −41.472 | 73.101 | 1.00 | 189.01 | C |
| ATOM | 1175 | C | ALA | A | 175 | −55.278 | −41.238 | 71.602 | 1.00 | 189.30 | C |
| ATOM | 1176 | O | ALA | A | 175 | −56.287 | −40.667 | 71.179 | 1.00 | 189.54 | O |
| ATOM | 1177 | CB | ALA | A | 175 | −55.750 | −42.751 | 73.528 | 1.00 | 188.91 | C |
| ATOM | 1178 | N | LEU | A | 176 | −54.308 | −41.690 | 70.812 | 1.00 | 189.42 | N |
| ATOM | 1179 | CA | LEU | A | 176 | −54.304 | −41.512 | 69.370 | 1.00 | 189.44 | C |
| ATOM | 1180 | C | LEU | A | 176 | −53.805 | −42.792 | 68.727 | 1.00 | 189.85 | C |
| ATOM | 1181 | O | LEU | A | 176 | −52.636 | −43.141 | 68.862 | 1.00 | 189.96 | O |
| ATOM | 1182 | CB | LEU | A | 176 | −53.365 | −40.370 | 69.023 | 1.00 | 189.22 | C |
| ATOM | 1183 | CG | LEU | A | 176 | −52.493 | −40.528 | 67.784 | 1.00 | 189.17 | C |
| ATOM | 1184 | CD1 | LEU | A | 176 | −53.303 | −40.254 | 66.540 | 1.00 | 189.44 | C |
| ATOM | 1185 | CD2 | LEU | A | 176 | −51.275 | −39.623 | 67.847 | 1.00 | 189.15 | C |
| ATOM | 1186 | N | LEU | A | 177 | −54.676 | −43.499 | 68.022 | 1.00 | 190.34 | N |
| ATOM | 1187 | CA | LEU | A | 177 | −54.282 | −44.796 | 67.468 | 1.00 | 190.90 | C |
| ATOM | 1188 | C | LEU | A | 177 | −54.140 | −44.794 | 65.946 | 1.00 | 191.59 | C |
| ATOM | 1189 | O | LEU | A | 177 | −54.848 | −44.068 | 65.253 | 1.00 | 191.80 | O |
| ATOM | 1190 | CB | LEU | A | 177 | −55.264 | −45.882 | 67.901 | 1.00 | 190.61 | C |
| ATOM | 1191 | CG | LEU | A | 177 | −55.431 | −46.207 | 69.382 | 1.00 | 190.01 | C |
| ATOM | 1192 | CD1 | LEU | A | 177 | −56.446 | −47.314 | 69.488 | 1.00 | 189.35 | C |
| ATOM | 1193 | CD2 | LEU | A | 177 | −54.117 | −46.611 | 70.058 | 1.00 | 189.62 | C |
| ATOM | 1194 | N | VAL | A | 178 | −53.242 | −45.631 | 65.434 | 1.00 | 192.29 | N |
| ATOM | 1195 | CA | VAL | A | 178 | −52.959 | −45.685 | 64.008 | 1.00 | 193.12 | C |
| ATOM | 1196 | C | VAL | A | 178 | −52.639 | −47.102 | 63.605 | 1.00 | 194.04 | C |
| ATOM | 1197 | O | VAL | A | 178 | −51.817 | −47.732 | 64.268 | 1.00 | 193.96 | O |
| ATOM | 1198 | CB | VAL | A | 178 | −51.742 | −44.831 | 63.701 | 1.00 | 193.03 | C |
| ATOM | 1199 | CG1 | VAL | A | 178 | −50.698 | −45.018 | 64.782 | 1.00 | 192.65 | C |
| ATOM | 1200 | CG2 | VAL | A | 178 | −51.174 | −45.173 | 62.331 | 1.00 | 193.41 | C |
| ATOM | 1201 | N | CYS | A | 179 | −53.266 | −47.603 | 62.531 | 1.00 | 195.52 | N |
| ATOM | 1202 | CA | CYS | A | 179 | −53.026 | −49.000 | 62.067 | 1.00 | 197.43 | C |
| ATOM | 1203 | C | CYS | A | 179 | −53.491 | −49.376 | 60.650 | 1.00 | 197.98 | C |
| ATOM | 1204 | O | CYS | A | 179 | −54.189 | −48.603 | 60.008 | 1.00 | 198.17 | O |
| ATOM | 1205 | CB | CYS | A | 179 | −53.560 | −50.020 | 63.083 | 1.00 | 197.77 | C |
| ATOM | 1206 | SG | CYS | A | 179 | −55.008 | −49.476 | 64.047 | 1.00 | 200.46 | S |
| ATOM | 1207 | N | ARG | A | 180 | −53.120 | −50.580 | 60.200 | 1.00 | 198.93 | N |
| ATOM | 1208 | CA | ARG | A | 180 | −53.222 | −50.995 | 58.785 | 1.00 | 200.32 | C |
| ATOM | 1209 | C | ARG | A | 180 | −54.590 | −50.838 | 58.111 | 1.00 | 200.90 | C |
| ATOM | 1210 | O | ARG | A | 180 | −55.602 | −50.668 | 58.785 | 1.00 | 201.03 | O |
| ATOM | 1211 | CB | ARG | A | 180 | −52.686 | −52.419 | 58.591 | 1.00 | 200.37 | C |
| ATOM | 1212 | CG | ARG | A | 180 | −51.265 | −52.485 | 57.986 | 1.00 | 201.29 | C |
| ATOM | 1213 | CD | ARG | A | 180 | −50.706 | −53.926 | 57.892 | 1.00 | 201.01 | C |
| ATOM | 1214 | NE | ARG | A | 180 | −50.161 | −54.423 | 59.164 | 1.00 | 202.06 | N |
| ATOM | 1215 | CZ | ARG | A | 180 | −50.842 | −55.149 | 60.057 | 1.00 | 202.77 | C |
| ATOM | 1216 | NH1 | ARG | A | 180 | −50.258 | −55.551 | 61.183 | 1.00 | 202.94 | N |
| ATOM | 1217 | NH2 | ARG | A | 180 | −52.110 | −55.481 | 59.837 | 1.00 | 203.23 | N |
| ATOM | 1218 | N | GLU | A | 181 | −54.594 | −50.928 | 56.778 | 1.00 | 201.77 | N |
| ATOM | 1219 | CA | GLU | A | 181 | −55.780 | −50.704 | 55.936 | 1.00 | 202.90 | C |
| ATOM | 1220 | C | GLU | A | 181 | −57.002 | −51.575 | 56.285 | 1.00 | 203.29 | C |
| ATOM | 1221 | O | GLU | A | 181 | −57.250 | −52.590 | 55.633 | 1.00 | 203.33 | O |
| ATOM | 1222 | CB | GLU | A | 181 | −55.405 | −50.864 | 54.445 | 1.00 | 202.86 | C |
| ATOM | 1223 | CG | GLU | A | 181 | −55.108 | −49.536 | 53.685 | 1.00 | 203.71 | C |
| ATOM | 1224 | CD | GLU | A | 181 | −54.230 | −49.695 | 52.408 | 1.00 | 203.77 | C |
| ATOM | 1225 | OE1 | GLU | A | 181 | −54.635 | −49.220 | 51.319 | 1.00 | 204.37 | O |
| ATOM | 1226 | OE2 | GLU | A | 181 | −53.121 | −50.274 | 52.487 | 1.00 | 205.20 | O |
| ATOM | 1227 | N | GLY | A | 182 | −57.762 | −51.166 | 57.306 | 1.00 | 203.97 | N |
| ATOM | 1228 | CA | GLY | A | 182 | −58.969 | −51.894 | 57.729 | 1.00 | 204.71 | C |
| ATOM | 1229 | C | GLY | A | 182 | −59.389 | −51.786 | 59.194 | 1.00 | 205.29 | C |
| ATOM | 1230 | O | GLY | A | 182 | −59.213 | −52.722 | 59.970 | 1.00 | 205.04 | O |
| ATOM | 1231 | N | SER | A | 183 | −59.942 | −50.638 | 59.569 | 1.00 | 206.10 | N |
| ATOM | 1232 | CA | SER | A | 183 | −60.639 | −50.477 | 60.844 | 1.00 | 207.01 | C |
| ATOM | 1233 | C | SER | A | 183 | −62.107 | −50.275 | 60.525 | 1.00 | 207.93 | C |
| ATOM | 1234 | O | SER | A | 183 | −62.923 | −51.150 | 60.787 | 1.00 | 207.93 | O |
| ATOM | 1235 | CB | SER | A | 183 | −60.114 | −49.271 | 61.618 | 1.00 | 206.78 | C |
| ATOM | 1236 | OG | SER | A | 183 | −58.742 | −49.420 | 61.899 | 1.00 | 206.57 | O |
| ATOM | 1237 | N | LEU | A | 184 | −62.419 | −49.110 | 59.948 | 1.00 | 209.32 | N |
| ATOM | 1238 | CA | LEU | A | 184 | −63.728 | −48.779 | 59.338 | 1.00 | 210.38 | C |
| ATOM | 1239 | C | LEU | A | 184 | −64.402 | −49.936 | 58.549 | 1.00 | 211.35 | C |
| ATOM | 1240 | O | LEU | A | 184 | −65.627 | −49.913 | 58.339 | 1.00 | 211.53 | O |
| ATOM | 1241 | CB | LEU | A | 184 | −63.583 | −47.490 | 58.471 | 1.00 | 210.39 | C |
| ATOM | 1242 | CG | LEU | A | 184 | −64.282 | −47.125 | 57.135 | 1.00 | 209.98 | C |
| ATOM | 1243 | CD1 | LEU | A | 184 | −65.745 | −46.684 | 57.309 | 1.00 | 210.18 | C |
| ATOM | 1244 | CD2 | LEU | A | 184 | −63.491 | −46.050 | 56.359 | 1.00 | 210.16 | C |
| ATOM | 1245 | N | ALA | A | 185 | −63.607 | −50.937 | 58.140 | 1.00 | 212.32 | N |
| ATOM | 1246 | CA | ALA | A | 185 | −64.071 | −52.066 | 57.300 | 1.00 | 213.06 | C |
| ATOM | 1247 | C | ALA | A | 185 | −63.888 | −53.461 | 57.926 | 1.00 | 213.51 | C |
| ATOM | 1248 | O | ALA | A | 185 | −64.715 | −54.359 | 57.702 | 1.00 | 213.48 | O |
| ATOM | 1249 | CB | ALA | A | 185 | −63.407 | −52.012 | 55.905 | 1.00 | 213.03 | C |
| ATOM | 1250 | N | LYS | A | 186 | −62.803 | −53.634 | 58.688 | 1.00 | 214.11 | N |
| ATOM | 1251 | CA | LYS | A | 186 | −62.486 | −54.911 | 59.339 | 1.00 | 214.65 | C |
| ATOM | 1252 | C | LYS | A | 186 | −62.588 | −54.815 | 60.864 | 1.00 | 215.01 | C |
| ATOM | 1253 | O | LYS | A | 186 | −62.071 | −55.670 | 61.584 | 1.00 | 215.04 | O |
| ATOM | 1254 | CB | LYS | A | 186 | −61.100 | −55.427 | 58.915 | 1.00 | 214.74 | C |
| ATOM | 1255 | CG | LYS | A | 186 | −60.923 | −56.958 | 58.992 | 1.00 | 214.69 | C |
| ATOM | 1256 | CD | LYS | A | 186 | −60.788 | −57.609 | 57.618 | 1.00 | 214.92 | C |
| ATOM | 1257 | CE | LYS | A | 186 | −62.111 | −57.668 | 56.874 | 1.00 | 215.11 | C |
| ATOM | 1258 | NZ | LYS | A | 186 | −61.894 | −57.578 | 55.407 | 1.00 | 215.32 | N |
| ATOM | 1259 | N | GLU | A | 187 | −63.224 | −53.746 | 61.343 | 1.00 | 215.49 | N |
| ATOM | 1260 | CA | GLU | A | 187 | −63.785 | −53.685 | 62.707 | 1.00 | 215.94 | C |
| ATOM | 1261 | C | GLU | A | 187 | −65.281 | −53.300 | 62.658 | 1.00 | 215.88 | C |
| ATOM | 1262 | O | GLU | A | 187 | −65.950 | −53.193 | 63.696 | 1.00 | 215.92 | O |
| ATOM | 1263 | CB | GLU | A | 187 | −62.976 | −52.765 | 63.646 | 1.00 | 216.09 | C |
| ATOM | 1264 | CG | GLU | A | 187 | −62.188 | −53.498 | 64.754 | 1.00 | 216.81 | C |
| ATOM | 1265 | CD | GLU | A | 187 | −63.085 | −54.115 | 65.843 | 1.00 | 218.09 | C |
| ATOM | 1266 | OE1 | GLU | A | 187 | −63.005 | −55.346 | 66.050 | 1.00 | 218.45 | O |
| ATOM | 1267 | OE2 | GLU | A | 187 | −63.867 | −53.379 | 66.494 | 1.00 | 218.51 | O |
| ATOM | 1268 | N | LYS | A | 188 | −65.787 | −53.093 | 61.439 | 1.00 | 215.79 | N |
| ATOM | 1269 | CA | LYS | A | 188 | −67.228 | −53.056 | 61.176 | 1.00 | 215.55 | C |
| ATOM | 1270 | C | LYS | A | 188 | −67.718 | −54.506 | 61.098 | 1.00 | 215.40 | C |
| ATOM | 1271 | O | LYS | A | 188 | −68.903 | −54.794 | 61.300 | 1.00 | 215.49 | O |
| ATOM | 1272 | CB | LYS | A | 188 | −67.547 | −52.267 | 59.882 | 1.00 | 215.63 | C |
| ATOM | 1273 | CG | LYS | A | 188 | −67.746 | −53.089 | 58.582 | 1.00 | 215.44 | C |
| ATOM | 1274 | CD | LYS | A | 188 | −67.776 | −52.212 | 57.323 | 1.00 | 215.36 | C |
| ATOM | 1275 | CE | LYS | A | 188 | −68.813 | −51.090 | 57.394 | 1.00 | 215.08 | C |
| ATOM | 1276 | NZ | LYS | A | 188 | −68.553 | −50.033 | 56.374 | 1.00 | 215.00 | N |
| ATOM | 1277 | N | THR | A | 189 | −66.774 | −55.408 | 60.827 | 1.00 | 215.02 | N |
| ATOM | 1278 | CA | THR | A | 189 | −67.046 | −56.829 | 60.657 | 1.00 | 214.66 | C |
| ATOM | 1279 | C | THR | A | 189 | −66.302 | −57.694 | 61.695 | 1.00 | 214.21 | C |
| ATOM | 1280 | O | THR | A | 189 | −66.678 | −58.851 | 61.934 | 1.00 | 214.21 | O |
| ATOM | 1281 | CB | THR | A | 189 | −66.745 | −57.281 | 59.193 | 1.00 | 214.77 | C |
| ATOM | 1282 | OG1 | THR | A | 189 | −67.157 | −58.640 | 59.006 | 1.00 | 215.22 | O |
| ATOM | 1283 | CG2 | THR | A | 189 | −65.263 | −57.145 | 58.848 | 1.00 | 214.79 | C |
| ATOM | 1284 | N | GLN | A | 190 | −65.266 | −57.118 | 62.315 | 1.00 | 213.59 | N |
| ATOM | 1285 | CA | GLN | A | 190 | −64.457 | −57.815 | 63.329 | 1.00 | 212.97 | C |
| ATOM | 1286 | C | GLN | A | 190 | −64.883 | −57.488 | 64.763 | 1.00 | 212.31 | C |
| ATOM | 1287 | O | GLN | A | 190 | −65.461 | −56.427 | 65.037 | 1.00 | 212.30 | O |
| ATOM | 1288 | CB | GLN | A | 190 | −62.968 | −57.487 | 63.165 | 1.00 | 213.06 | C |
| ATOM | 1289 | CG | GLN | A | 190 | −62.021 | −58.641 | 63.452 | 1.00 | 213.56 | C |
| ATOM | 1290 | CD | GLN | A | 190 | −61.734 | −59.473 | 62.212 | 1.00 | 214.59 | C |
| ATOM | 1291 | OE1 | GLN | A | 190 | −61.083 | −59.006 | 61.273 | 1.00 | 215.00 | O |
| ATOM | 1292 | NE2 | GLN | A | 190 | −62.216 | −60.714 | 62.203 | 1.00 | 214.81 | N |
| ATOM | 1293 | N | THR | A | 191 | −64.585 | −58.414 | 65.671 | 1.00 | 211.33 | N |
| ATOM | 1294 | CA | THR | A | 191 | −64.808 | −58.223 | 67.101 | 1.00 | 210.24 | C |
| ATOM | 1295 | C | THR | A | 191 | −63.455 | −58.013 | 67.781 | 1.00 | 209.30 | C |
| ATOM | 1296 | O | THR | A | 191 | −62.526 | −58.796 | 67.565 | 1.00 | 209.33 | O |
| ATOM | 1297 | CB | THR | A | 191 | −65.600 | −59.425 | 67.730 | 1.00 | 210.37 | C |
| ATOM | 1298 | OG1 | THR | A | 191 | −65.354 | −59.499 | 69.142 | 1.00 | 210.46 | O |
| ATOM | 1299 | CG2 | THR | A | 191 | −65.217 | −60.763 | 67.080 | 1.00 | 210.31 | C |
| ATOM | 1300 | N | LEU | A | 192 | −63.334 | −56.940 | 68.564 | 1.00 | 207.94 | N |
| ATOM | 1301 | CA | LEU | A | 192 | −62.120 | −56.691 | 69.354 | 1.00 | 206.68 | C |
| ATOM | 1302 | C | LEU | A | 192 | −62.425 | −56.065 | 70.709 | 1.00 | 205.70 | C |
| ATOM | 1303 | O | LEU | A | 192 | −63.189 | −55.104 | 70.790 | 1.00 | 205.83 | O |
| ATOM | 1304 | CB | LEU | A | 192 | −61.123 | −55.814 | 68.588 | 1.00 | 206.75 | C |
| ATOM | 1305 | CG | LEU | A | 192 | −60.008 | −56.502 | 67.785 | 1.00 | 206.91 | C |
| ATOM | 1306 | CD1 | LEU | A | 192 | −59.366 | −55.552 | 66.757 | 1.00 | 207.22 | C |
| ATOM | 1307 | CD2 | LEU | A | 192 | −58.950 | −57.086 | 68.718 | 1.00 | 206.74 | C |
| ATOM | 1308 | N | HIS | A | 193 | −61.827 | −56.617 | 71.765 | 1.00 | 204.26 | N |
| ATOM | 1309 | CA | HIS | A | 193 | −61.988 | −56.082 | 73.116 | 1.00 | 202.85 | C |
| ATOM | 1310 | C | HIS | A | 193 | −60.783 | −55.262 | 73.513 | 1.00 | 201.68 | C |
| ATOM | 1311 | O | HIS | A | 193 | −59.770 | −55.797 | 73.974 | 1.00 | 201.49 | O |
| ATOM | 1312 | CB | HIS | A | 193 | −62.201 | −57.201 | 74.122 | 1.00 | 203.05 | C |
| ATOM | 1313 | CG | HIS | A | 193 | −63.585 | −57.756 | 74.110 | 1.00 | 203.48 | C |
| ATOM | 1314 | ND1 | HIS | A | 193 | −64.042 | −58.589 | 73.111 | 1.00 | 204.12 | N |
| ATOM | 1315 | CD2 | HIS | A | 193 | −64.615 | −57.597 | 74.971 | 1.00 | 203.92 | C |
| ATOM | 1316 | CE1 | HIS | A | 193 | −65.295 | −58.924 | 73.359 | 1.00 | 204.31 | C |
| ATOM | 1317 | NE2 | HIS | A | 193 | −65.667 | −58.336 | 74.482 | 1.00 | 204.61 | N |
| ATOM | 1318 | N | LYS | A | 194 | −60.901 | −53.955 | 73.330 | 1.00 | 200.25 | N |
| ATOM | 1319 | CA | LYS | A | 194 | −59.780 | −53.068 | 73.571 | 1.00 | 198.91 | C |
| ATOM | 1320 | C | LYS | A | 194 | −60.117 | −51.842 | 74.406 | 1.00 | 197.81 | C |
| ATOM | 1321 | O | LYS | A | 194 | −61.153 | −51.202 | 74.215 | 1.00 | 197.67 | O |
| ATOM | 1322 | CB | LYS | A | 194 | −59.032 | −52.697 | 72.259 | 1.00 | 199.23 | C |
| ATOM | 1323 | CG | LYS | A | 194 | −59.847 | −52.204 | 71.017 | 1.00 | 199.04 | C |
| ATOM | 1324 | CD | LYS | A | 194 | −58.910 | −52.066 | 69.769 | 1.00 | 198.99 | C |
| ATOM | 1325 | CE | LYS | A | 194 | −59.626 | −51.579 | 68.497 | 1.00 | 198.69 | C |
| ATOM | 1326 | NZ | LYS | A | 194 | −58.794 | −51.745 | 67.258 | 1.00 | 197.91 | N |
| ATOM | 1327 | N | PHE | A | 195 | −59.226 | −51.539 | 75.345 | 1.00 | 196.43 | N |
| ATOM | 1328 | CA | PHE | A | 195 | −59.324 | −50.327 | 76.141 | 1.00 | 195.22 | C |
| ATOM | 1329 | C | PHE | A | 195 | −58.027 | −49.556 | 76.119 | 1.00 | 193.91 | C |
| ATOM | 1330 | O | PHE | A | 195 | −56.974 | −50.068 | 75.749 | 1.00 | 193.48 | O |
| ATOM | 1331 | CB | PHE | A | 195 | −59.652 | −50.642 | 77.598 | 1.00 | 195.71 | C |
| ATOM | 1332 | CG | PHE | A | 195 | −60.709 | −51.675 | 77.769 | 1.00 | 196.63 | C |
| ATOM | 1333 | CD1 | PHE | A | 195 | −62.058 | −51.323 | 77.711 | 1.00 | 197.72 | C |
| ATOM | 1334 | CD2 | PHE | A | 195 | −60.364 | −53.005 | 77.992 | 1.00 | 197.37 | C |
| ATOM | 1335 | CE1 | PHE | A | 195 | −63.060 | −52.289 | 77.872 | 1.00 | 198.12 | C |
| ATOM | 1336 | CE2 | PHE | A | 195 | −61.350 | −53.979 | 78.153 | 1.00 | 198.10 | C |
| ATOM | 1337 | CZ | PHE | A | 195 | −62.705 | −53.621 | 78.094 | 1.00 | 197.78 | C |
| ATOM | 1338 | N | ILE | A | 196 | −58.120 | −48.311 | 76.544 | 1.00 | 192.50 | N |
| ATOM | 1339 | CA | ILE | A | 196 | −56.946 | −47.512 | 76.752 | 1.00 | 191.13 | C |
| ATOM | 1340 | C | ILE | A | 196 | −56.802 | −47.180 | 78.247 | 1.00 | 190.24 | C |
| ATOM | 1341 | O | ILE | A | 196 | −57.635 | −46.501 | 78.860 | 1.00 | 189.99 | O |
| ATOM | 1342 | CB | ILE | A | 196 | −56.869 | −46.306 | 75.739 | 1.00 | 191.15 | C |
| ATOM | 1343 | CG1 | ILE | A | 196 | −55.966 | −45.170 | 76.253 | 1.00 | 191.45 | C |
| ATOM | 1344 | CG2 | ILE | A | 196 | −58.267 | −45.849 | 75.268 | 1.00 | 190.71 | C |
| ATOM | 1345 | CD1 | ILE | A | 196 | −56.651 | −44.089 | 77.073 | 1.00 | 191.64 | C |
| ATOM | 1346 | N | LEU | A | 197 | −55.748 | −47.738 | 78.825 | 1.00 | 189.12 | N |
| ATOM | 1347 | CA | LEU | A | 197 | −55.392 | −47.528 | 80.210 | 1.00 | 188.04 | C |
| ATOM | 1348 | C | LEU | A | 197 | −54.284 | −46.486 | 80.237 | 1.00 | 188.07 | C |
| ATOM | 1349 | O | LEU | A | 197 | −53.244 | −46.680 | 79.604 | 1.00 | 188.19 | O |
| ATOM | 1350 | CB | LEU | A | 197 | −54.870 | −48.833 | 80.796 | 1.00 | 187.76 | C |
| ATOM | 1351 | CG | LEU | A | 197 | −55.797 | −50.027 | 81.031 | 1.00 | 186.95 | C |
| ATOM | 1352 | CD1 | LEU | A | 197 | −56.766 | −50.300 | 79.888 | 1.00 | 186.14 | C |
| ATOM | 1353 | CD2 | LEU | A | 197 | −54.962 | −51.255 | 81.329 | 1.00 | 187.38 | C |
| ATOM | 1354 | N | LEU | A | 198 | −54.496 | −45.392 | 80.962 | 1.00 | 187.81 | N |
| ATOM | 1355 | CA | LEU | A | 198 | −53.553 | −44.276 | 80.957 | 1.00 | 187.71 | C |
| ATOM | 1356 | C | LEU | A | 198 | −52.740 | −44.223 | 82.249 | 1.00 | 187.92 | C |
| ATOM | 1357 | O | LEU | A | 198 | −53.089 | −43.483 | 83.155 | 1.00 | 188.01 | O |
| ATOM | 1358 | CB | LEU | A | 198 | −54.328 | −42.966 | 80.756 | 1.00 | 187.56 | C |
| ATOM | 1359 | CG | LEU | A | 198 | −53.702 | −41.714 | 80.122 | 1.00 | 187.06 | C |
| ATOM | 1360 | CD1 | LEU | A | 198 | −54.762 | −40.638 | 79.870 | 1.00 | 186.05 | C |
| ATOM | 1361 | CD2 | LEU | A | 198 | −52.551 | −41.142 | 80.942 | 1.00 | 186.26 | C |
| ATOM | 1362 | N | PHE | A | 199 | −51.658 | −44.993 | 82.336 | 1.00 | 188.34 | N |
| ATOM | 1363 | CA | PHE | A | 199 | −50.891 | −45.106 | 83.588 | 1.00 | 189.15 | C |
| ATOM | 1364 | C | PHE | A | 199 | −50.124 | −43.832 | 83.924 | 1.00 | 190.22 | C |
| ATOM | 1365 | O | PHE | A | 199 | −48.965 | −43.679 | 83.531 | 1.00 | 190.28 | O |
| ATOM | 1366 | CB | PHE | A | 199 | −49.940 | −46.299 | 83.534 | 1.00 | 188.60 | C |
| ATOM | 1367 | CG | PHE | A | 199 | −50.611 | −47.622 | 83.766 | 1.00 | 188.03 | C |
| ATOM | 1368 | CD1 | PHE | A | 199 | −51.390 | −48.216 | 82.777 | 1.00 | 187.61 | C |
| ATOM | 1369 | CD2 | PHE | A | 199 | −50.458 | −48.286 | 84.967 | 1.00 | 187.13 | C |
| ATOM | 1370 | CE1 | PHE | A | 199 | −52.015 | −49.453 | 82.993 | 1.00 | 187.04 | C |
| ATOM | 1371 | CE2 | PHE | A | 199 | −51.079 | −49.514 | 85.182 | 1.00 | 186.59 | C |
| ATOM | 1372 | CZ | PHE | A | 199 | −51.855 | −50.099 | 84.195 | 1.00 | 186.73 | C |
| ATOM | 1373 | N | ALA | A | 200 | −50.772 | −42.944 | 84.688 | 1.00 | 191.63 | N |
| ATOM | 1374 | CA | ALA | A | 200 | −50.368 | −41.517 | 84.790 | 1.00 | 192.78 | C |
| ATOM | 1375 | C | ALA | A | 200 | −50.242 | −40.913 | 86.204 | 1.00 | 193.40 | C |
| ATOM | 1376 | O | ALA | A | 200 | −51.027 | −41.242 | 87.103 | 1.00 | 193.69 | O |
| ATOM | 1377 | CB | ALA | A | 200 | −51.304 | −40.648 | 83.942 | 1.00 | 192.79 | C |
| ATOM | 1378 | N | VAL | A | 201 | −49.287 | −39.982 | 86.353 | 1.00 | 193.95 | N |
| ATOM | 1379 | CA | VAL | A | 201 | −48.829 | −39.464 | 87.658 | 1.00 | 194.31 | C |
| ATOM | 1380 | C | VAL | A | 201 | −48.883 | −37.922 | 87.786 | 1.00 | 194.61 | C |
| ATOM | 1381 | O | VAL | A | 201 | −47.850 | −37.287 | 88.019 | 1.00 | 194.63 | O |
| ATOM | 1382 | CB | VAL | A | 201 | −47.347 | −39.902 | 87.946 | 1.00 | 194.31 | C |
| ATOM | 1383 | CG1 | VAL | A | 201 | −47.096 | −40.011 | 89.440 | 1.00 | 194.19 | C |
| ATOM | 1384 | CG2 | VAL | A | 201 | −46.971 | −41.216 | 87.222 | 1.00 | 194.15 | C |
| ATOM | 1385 | N | PHE | A | 202 | −50.063 | −37.321 | 87.643 | 1.00 | 195.03 | N |
| ATOM | 1386 | CA | PHE | A | 202 | −50.186 | −35.857 | 87.741 | 1.00 | 195.62 | C |
| ATOM | 1387 | C | PHE | A | 202 | −49.821 | −35.386 | 89.145 | 1.00 | 196.07 | C |
| ATOM | 1388 | O | PHE | A | 202 | −50.267 | −35.982 | 90.132 | 1.00 | 196.31 | O |
| ATOM | 1389 | CB | PHE | A | 202 | −51.611 | −35.369 | 87.468 | 1.00 | 195.67 | C |
| ATOM | 1390 | CG | PHE | A | 202 | −52.365 | −36.151 | 86.433 | 1.00 | 195.87 | C |
| ATOM | 1391 | CD1 | PHE | A | 202 | −52.656 | −37.505 | 86.621 | 1.00 | 196.21 | C |
| ATOM | 1392 | CD2 | PHE | A | 202 | −52.855 | −35.510 | 85.298 | 1.00 | 195.84 | C |
| ATOM | 1393 | CE1 | PHE | A | 202 | −53.383 | −38.221 | 85.675 | 1.00 | 196.14 | C |
| ATOM | 1394 | CE2 | PHE | A | 202 | −53.588 | −36.211 | 84.349 | 1.00 | 196.00 | C |
| ATOM | 1395 | CZ | PHE | A | 202 | −53.851 | −37.573 | 84.537 | 1.00 | 196.10 | C |
| ATOM | 1396 | N | ASP | A | 203 | −49.043 | −34.304 | 89.235 | 1.00 | 196.48 | N |
| ATOM | 1397 | CA | ASP | A | 203 | −48.616 | −33.748 | 90.531 | 1.00 | 196.77 | C |
| ATOM | 1398 | C | ASP | A | 203 | −49.139 | −32.327 | 90.771 | 1.00 | 196.76 | C |
| ATOM | 1399 | O | ASP | A | 203 | −48.524 | −31.367 | 90.320 | 1.00 | 196.80 | O |
| ATOM | 1400 | CB | ASP | A | 203 | −47.082 | −33.776 | 90.641 | 1.00 | 196.86 | C |
| ATOM | 1401 | CG | ASP | A | 203 | −46.589 | −33.413 | 92.032 | 1.00 | 197.33 | C |
| ATOM | 1402 | OD1 | ASP | A | 203 | −46.187 | −34.332 | 92.786 | 1.00 | 197.59 | O |
| ATOM | 1403 | OD2 | ASP | A | 203 | −46.616 | −32.209 | 92.375 | 1.00 | 197.75 | O |
| ATOM | 1404 | N | GLU | A | 204 | −50.243 | −32.193 | 91.506 | 1.00 | 196.82 | N |
| ATOM | 1405 | CA | GLU | A | 204 | −50.934 | −30.897 | 91.657 | 1.00 | 197.03 | C |
| ATOM | 1406 | C | GLU | A | 204 | −50.122 | −29.699 | 92.178 | 1.00 | 197.41 | C |
| ATOM | 1407 | O | GLU | A | 204 | −50.657 | −28.593 | 92.304 | 1.00 | 197.34 | O |
| ATOM | 1408 | CB | GLU | A | 204 | −52.235 | −31.063 | 92.448 | 1.00 | 196.95 | C |
| ATOM | 1409 | CG | GLU | A | 204 | −53.495 | −30.857 | 91.609 | 1.00 | 196.32 | C |
| ATOM | 1410 | CD | GLU | A | 204 | −53.442 | −31.536 | 90.245 | 1.00 | 195.14 | C |
| ATOM | 1411 | OE1 | GLU | A | 204 | −53.687 | −30.836 | 89.234 | 1.00 | 194.19 | O |
| ATOM | 1412 | OE2 | GLU | A | 204 | −53.163 | −32.757 | 90.187 | 1.00 | 194.28 | O |
| ATOM | 1413 | N | GLY | A | 205 | −48.842 | −29.922 | 92.471 | 1.00 | 197.90 | N |
| ATOM | 1414 | CA | GLY | A | 205 | −47.909 | −28.842 | 92.797 | 1.00 | 198.66 | C |
| ATOM | 1415 | C | GLY | A | 205 | −47.252 | −28.288 | 91.545 | 1.00 | 199.21 | C |
| ATOM | 1416 | O | GLY | A | 205 | −46.743 | −27.164 | 91.541 | 1.00 | 199.20 | O |
| ATOM | 1417 | N | LYS | A | 206 | −47.262 | −29.104 | 90.488 | 1.00 | 199.86 | N |
| ATOM | 1418 | CA | LYS | A | 206 | −46.789 | −28.737 | 89.144 | 1.00 | 200.44 | C |
| ATOM | 1419 | C | LYS | A | 206 | −47.992 | −28.566 | 88.199 | 1.00 | 200.78 | C |
| ATOM | 1420 | O | LYS | A | 206 | −47.908 | −28.851 | 87.001 | 1.00 | 200.69 | O |
| ATOM | 1421 | CB | LYS | A | 206 | −45.845 | −29.822 | 88.580 | 1.00 | 200.48 | C |
| ATOM | 1422 | CG | LYS | A | 206 | −44.781 | −30.382 | 89.546 | 1.00 | 200.57 | C |
| ATOM | 1423 | CD | LYS | A | 206 | −43.377 | −29.813 | 89.305 | 1.00 | 200.39 | C |
| ATOM | 1424 | CE | LYS | A | 206 | −43.232 | −28.354 | 89.738 | 1.00 | 200.51 | C |
| ATOM | 1425 | NZ | LYS | A | 206 | −43.635 | −28.123 | 91.156 | 1.00 | 200.70 | N |
| ATOM | 1426 | N | SER | A | 207 | −49.106 | −28.090 | 88.754 | 1.00 | 201.31 | N |
| ATOM | 1427 | CA | SER | A | 207 | −50.359 | −27.927 | 88.019 | 1.00 | 201.80 | C |
| ATOM | 1428 | C | SER | A | 207 | −50.320 | −26.656 | 87.179 | 1.00 | 202.04 | C |
| ATOM | 1429 | O | SER | A | 207 | −49.260 | −26.073 | 86.952 | 1.00 | 202.10 | O |
| ATOM | 1430 | CB | SER | A | 207 | −51.534 | −27.877 | 89.015 | 1.00 | 201.83 | C |
| ATOM | 1431 | OG | SER | A | 207 | −52.793 | −27.805 | 88.363 | 1.00 | 202.09 | O |
| ATOM | 1432 | N | TRP | A | 208 | −51.481 | −26.236 | 86.701 | 1.00 | 202.41 | N |
| ATOM | 1433 | CA | TRP | A | 208 | −51.628 | −24.884 | 86.227 | 1.00 | 202.76 | C |
| ATOM | 1434 | C | TRP | A | 208 | −51.649 | −23.975 | 87.457 | 1.00 | 203.16 | C |
| ATOM | 1435 | O | TRP | A | 208 | −51.381 | −22.775 | 87.341 | 1.00 | 203.29 | O |
| ATOM | 1436 | CB | TRP | A | 208 | −52.926 | −24.740 | 85.438 | 1.00 | 202.73 | C |
| ATOM | 1437 | CG | TRP | A | 208 | −54.154 | −24.834 | 86.285 | 1.00 | 202.35 | C |
| ATOM | 1438 | CD1 | TRP | A | 208 | −54.890 | −25.951 | 86.547 | 1.00 | 201.92 | C |
| ATOM | 1439 | CD2 | TRP | A | 208 | −54.783 | −23.760 | 86.988 | 1.00 | 202.48 | C |
| ATOM | 1440 | NE1 | TRP | A | 208 | −55.941 | −25.642 | 87.368 | 1.00 | 201.63 | N |
| ATOM | 1441 | CE2 | TRP | A | 208 | −55.899 | −24.302 | 87.659 | 1.00 | 202.46 | C |
| ATOM | 1442 | CE3 | TRP | A | 208 | −54.512 | −22.381 | 87.116 | 1.00 | 202.79 | C |
| ATOM | 1443 | CZ2 | TRP | A | 208 | −56.755 | −23.513 | 88.455 | 1.00 | 203.16 | C |
| ATOM | 1444 | CZ3 | TRP | A | 208 | −55.359 | −21.593 | 87.909 | 1.00 | 202.51 | C |
| ATOM | 1445 | CH2 | TRP | A | 208 | −56.469 | −22.164 | 88.567 | 1.00 | 202.80 | C |
| ATOM | 1446 | N | HIS | A | 209 | −51.940 | −24.572 | 88.625 | 1.00 | 203.54 | N |
| ATOM | 1447 | CA | HIS | A | 209 | −52.324 | −23.850 | 89.861 | 1.00 | 204.02 | C |
| ATOM | 1448 | C | HIS | A | 209 | −51.242 | −23.701 | 90.948 | 1.00 | 203.80 | C |
| ATOM | 1449 | O | HIS | A | 209 | −50.335 | −24.534 | 91.053 | 1.00 | 203.86 | O |
| ATOM | 1450 | CB | HIS | A | 209 | −53.591 | −24.488 | 90.473 | 1.00 | 204.34 | C |
| ATOM | 1451 | CG | HIS | A | 209 | −53.399 | −25.063 | 91.851 | 1.00 | 205.91 | C |
| ATOM | 1452 | ND1 | HIS | A | 209 | −52.842 | −26.307 | 92.076 | 1.00 | 207.18 | N |
| ATOM | 1453 | CD2 | HIS | A | 209 | −53.717 | −24.569 | 93.075 | 1.00 | 207.00 | C |
| ATOM | 1454 | CE1 | HIS | A | 209 | −52.816 | −26.548 | 93.377 | 1.00 | 207.58 | C |
| ATOM | 1455 | NE2 | HIS | A | 209 | −53.341 | −25.509 | 94.005 | 1.00 | 207.56 | N |
| ATOM | 1456 | N | SER | A | 210 | −51.377 | −22.643 | 91.757 | 1.00 | 203.50 | N |
| ATOM | 1457 | CA | SER | A | 210 | −50.536 | −22.409 | 92.934 | 1.00 | 203.18 | C |
| ATOM | 1458 | C | SER | A | 210 | −51.397 | −22.358 | 94.193 | 1.00 | 203.10 | C |
| ATOM | 1459 | O | SER | A | 210 | −50.884 | −22.273 | 95.307 | 1.00 | 203.00 | O |
| ATOM | 1460 | CB | SER | A | 210 | −49.749 | −21.106 | 92.786 | 1.00 | 203.10 | C |
| ATOM | 1461 | OG | SER | A | 210 | −50.618 | −20.008 | 92.579 | 1.00 | 202.81 | O |
| ATOM | 1462 | N | SER | A | 224 | −50.093 | −22.329 | 111.386 | 1.00 | 212.81 | N |
| ATOM | 1463 | CA | SER | A | 224 | −50.460 | −21.763 | 110.085 | 1.00 | 212.76 | C |
| ATOM | 1464 | C | SER | A | 224 | −50.376 | −22.770 | 108.914 | 1.00 | 212.74 | C |
| ATOM | 1465 | O | SER | A | 224 | −50.027 | −23.943 | 109.113 | 1.00 | 212.69 | O |
| ATOM | 1466 | CB | SER | A | 224 | −49.668 | −20.471 | 109.802 | 1.00 | 212.75 | C |
| ATOM | 1467 | OG | SER | A | 224 | −48.486 | −20.383 | 110.585 | 1.00 | 212.55 | O |
| ATOM | 1468 | N | ALA | A | 225 | −50.719 | −22.292 | 107.712 | 1.00 | 212.66 | N |
| ATOM | 1469 | CA | ALA | A | 225 | −50.812 | −23.091 | 106.483 | 1.00 | 212.64 | C |
| ATOM | 1470 | C | ALA | A | 225 | −50.049 | −24.427 | 106.520 | 1.00 | 212.74 | C |
| ATOM | 1471 | O | ALA | A | 225 | −48.815 | −24.441 | 106.537 | 1.00 | 212.77 | O |
| ATOM | 1472 | CB | ALA | A | 225 | −50.384 | −22.246 | 105.278 | 1.00 | 212.53 | C |
| ATOM | 1473 | N | ARG | A | 226 | −50.800 | −25.537 | 106.536 | 1.00 | 212.84 | N |
| ATOM | 1474 | CA | ARG | A | 226 | −50.246 | −26.907 | 106.649 | 1.00 | 212.86 | C |
| ATOM | 1475 | C | ARG | A | 226 | −51.027 | −27.975 | 105.823 | 1.00 | 212.72 | C |
| ATOM | 1476 | O | ARG | A | 226 | −51.888 | −28.674 | 106.376 | 1.00 | 212.87 | O |
| ATOM | 1477 | CB | ARG | A | 226 | −50.179 | −27.330 | 108.141 | 1.00 | 212.89 | C |
| ATOM | 1478 | CG | ARG | A | 226 | −49.016 | −28.274 | 108.558 | 1.00 | 213.08 | C |
| ATOM | 1479 | CD | ARG | A | 226 | −49.078 | −29.682 | 107.925 | 1.00 | 213.60 | C |
| ATOM | 1480 | NE | ARG | A | 226 | −48.367 | −29.757 | 106.638 | 1.00 | 213.95 | N |
| ATOM | 1481 | CZ | ARG | A | 226 | −48.646 | −30.615 | 105.651 | 1.00 | 214.08 | C |
| ATOM | 1482 | NH1 | ARG | A | 226 | −49.639 | −31.488 | 105.768 | 1.00 | 214.33 | N |
| ATOM | 1483 | NH2 | ARG | A | 226 | −47.937 | −30.592 | 104.527 | 1.00 | 213.96 | N |
| ATOM | 1484 | N | ALA | A | 227 | −50.727 | −28.097 | 104.521 | 1.00 | 212.37 | N |
| ATOM | 1485 | CA | ALA | A | 227 | −51.252 | −29.186 | 103.654 | 1.00 | 212.05 | C |
| ATOM | 1486 | C | ALA | A | 227 | −51.008 | −28.952 | 102.151 | 1.00 | 211.86 | C |
| ATOM | 1487 | O | ALA | A | 227 | −51.956 | −28.678 | 101.407 | 1.00 | 211.89 | O |
| ATOM | 1488 | CB | ALA | A | 227 | −52.754 | −29.450 | 103.922 | 1.00 | 211.97 | C |
| ATOM | 1489 | N | TRP | A | 228 | −49.751 | −29.085 | 101.710 | 1.00 | 211.62 | N |
| ATOM | 1490 | CA | TRP | A | 228 | −49.336 | −28.724 | 100.325 | 1.00 | 211.22 | C |
| ATOM | 1491 | C | TRP | A | 228 | −49.700 | −29.824 | 99.273 | 1.00 | 211.02 | C |
| ATOM | 1492 | O | TRP | A | 228 | −49.883 | −30.988 | 99.657 | 1.00 | 211.24 | O |
| ATOM | 1493 | CB | TRP | A | 228 | −47.839 | −28.311 | 100.268 | 1.00 | 211.51 | C |
| ATOM | 1494 | CG | TRP | A | 228 | −47.262 | −27.593 | 101.524 | 1.00 | 211.92 | C |
| ATOM | 1495 | CD1 | TRP | A | 228 | −46.306 | −28.089 | 102.375 | 1.00 | 212.07 | C |
| ATOM | 1496 | CD2 | TRP | A | 228 | −47.597 | −26.275 | 102.036 | 1.00 | 212.26 | C |
| ATOM | 1497 | NE1 | TRP | A | 228 | −46.033 | −27.179 | 103.376 | 1.00 | 212.17 | N |
| ATOM | 1498 | CE2 | TRP | A | 228 | −46.807 | −26.062 | 103.195 | 1.00 | 212.13 | C |
| ATOM | 1499 | CE3 | TRP | A | 228 | −48.487 | −25.265 | 101.633 | 1.00 | 212.16 | C |
| ATOM | 1500 | CZ2 | TRP | A | 228 | −46.879 | −24.878 | 103.952 | 1.00 | 211.76 | C |
| ATOM | 1501 | CZ3 | TRP | A | 228 | −48.555 | −24.083 | 102.393 | 1.00 | 211.89 | C |
| ATOM | 1502 | CH2 | TRP | A | 228 | −47.753 | −23.906 | 103.535 | 1.00 | 211.78 | C |
| ATOM | 1503 | N | PRO | A | 229 | −49.809 | −29.461 | 97.958 | 1.00 | 210.43 | N |
| ATOM | 1504 | CA | PRO | A | 229 | −50.500 | −30.263 | 96.893 | 1.00 | 210.03 | C |
| ATOM | 1505 | C | PRO | A | 229 | −50.000 | −31.699 | 96.562 | 1.00 | 209.21 | C |
| ATOM | 1506 | O | PRO | A | 229 | −48.810 | −31.987 | 96.729 | 1.00 | 208.97 | O |
| ATOM | 1507 | CB | PRO | A | 229 | −50.395 | −29.357 | 95.656 | 1.00 | 209.91 | C |
| ATOM | 1508 | CG | PRO | A | 229 | −50.130 | −27.978 | 96.203 | 1.00 | 210.02 | C |
| ATOM | 1509 | CD | PRO | A | 229 | −49.266 | −28.206 | 97.397 | 1.00 | 210.28 | C |
| ATOM | 1510 | N | LYS | A | 230 | −50.909 | −32.568 | 96.078 | 1.00 | 208.56 | N |
| ATOM | 1511 | CA | LYS | A | 230 | −50.596 | −34.002 | 95.831 | 1.00 | 207.97 | C |
| ATOM | 1512 | C | LYS | A | 230 | −51.624 | −34.876 | 95.026 | 1.00 | 207.87 | C |
| ATOM | 1513 | O | LYS | A | 230 | −52.831 | −34.613 | 95.070 | 1.00 | 207.83 | O |
| ATOM | 1514 | CB | LYS | A | 230 | −50.278 | −34.681 | 97.176 | 1.00 | 208.06 | C |
| ATOM | 1515 | CG | LYS | A | 230 | −49.178 | −35.749 | 97.152 | 1.00 | 207.84 | C |
| ATOM | 1516 | CD | LYS | A | 230 | −47.847 | −35.212 | 96.618 | 1.00 | 207.04 | C |
| ATOM | 1517 | CE | LYS | A | 230 | −47.455 | −35.864 | 95.286 | 1.00 | 206.14 | C |
| ATOM | 1518 | NZ | LYS | A | 230 | −48.250 | −35.428 | 94.107 | 1.00 | 204.46 | N |
| ATOM | 1519 | N | MET | A | 231 | −51.097 | −35.885 | 94.296 | 1.00 | 207.48 | N |
| ATOM | 1520 | CA | MET | A | 231 | −51.792 | −37.056 | 93.643 | 1.00 | 207.13 | C |
| ATOM | 1521 | C | MET | A | 231 | −50.674 | −37.990 | 93.087 | 1.00 | 206.74 | C |
| ATOM | 1522 | O | MET | A | 231 | −49.499 | −37.708 | 93.342 | 1.00 | 206.60 | O |
| ATOM | 1523 | CB | MET | A | 231 | −52.776 | −36.645 | 92.521 | 1.00 | 207.26 | C |
| ATOM | 1524 | CG | MET | A | 231 | −54.313 | −36.737 | 92.848 | 1.00 | 207.51 | C |
| ATOM | 1525 | SD | MET | A | 231 | −55.434 | −36.570 | 91.381 | 1.00 | 207.73 | S |
| ATOM | 1526 | CE | MET | A | 231 | −57.021 | −36.195 | 92.144 | 1.00 | 207.35 | C |
| ATOM | 1527 | N | HIS | A | 232 | −51.012 | −39.069 | 92.344 | 1.00 | 206.31 | N |
| ATOM | 1528 | CA | HIS | A | 232 | −50.001 | −40.055 | 91.791 | 1.00 | 205.95 | C |
| ATOM | 1529 | C | HIS | A | 232 | −50.533 | −41.233 | 90.924 | 1.00 | 205.03 | C |
| ATOM | 1530 | O | HIS | A | 232 | −49.898 | −42.290 | 90.869 | 1.00 | 204.77 | O |
| ATOM | 1531 | CB | HIS | A | 232 | −49.142 | −40.653 | 92.951 | 1.00 | 206.11 | C |
| ATOM | 1532 | CG | HIS | A | 232 | −48.027 | −41.578 | 92.516 | 1.00 | 206.29 | C |
| ATOM | 1533 | ND1 | HIS | A | 232 | −48.251 | −42.851 | 92.025 | 1.00 | 205.99 | N |
| ATOM | 1534 | CD2 | HIS | A | 232 | −46.679 | −41.428 | 92.555 | 1.00 | 205.99 | C |
| ATOM | 1535 | CE1 | HIS | A | 232 | −47.094 | −43.427 | 91.750 | 1.00 | 205.65 | C |
| ATOM | 1536 | NE2 | HIS | A | 232 | −46.124 | −42.585 | 92.061 | 1.00 | 205.52 | N |
| ATOM | 1537 | N | THR | A | 233 | −51.662 | −41.105 | 90.236 | 1.00 | 204.21 | N |
| ATOM | 1538 | CA | THR | A | 233 | −52.367 | −42.362 | 89.931 | 1.00 | 203.58 | C |
| ATOM | 1539 | C | THR | A | 233 | −53.154 | −42.632 | 88.623 | 1.00 | 203.15 | C |
| ATOM | 1540 | O | THR | A | 233 | −53.659 | −41.720 | 87.963 | 1.00 | 202.88 | O |
| ATOM | 1541 | CB | THR | A | 233 | −53.227 | −42.725 | 91.137 | 1.00 | 203.65 | C |
| ATOM | 1542 | OG1 | THR | A | 233 | −53.606 | −41.513 | 91.794 | 1.00 | 203.80 | O |
| ATOM | 1543 | CG2 | THR | A | 233 | −52.419 | −43.518 | 92.112 | 1.00 | 203.49 | C |
| ATOM | 1544 | N | VAL | A | 234 | −53.247 | −43.930 | 88.307 | 1.00 | 202.78 | N |
| ATOM | 1545 | CA | VAL | A | 234 | −53.905 | −44.493 | 87.114 | 1.00 | 202.35 | C |
| ATOM | 1546 | C | VAL | A | 234 | −55.168 | −43.805 | 86.631 | 1.00 | 202.51 | C |
| ATOM | 1547 | O | VAL | A | 234 | −56.086 | −43.515 | 87.402 | 1.00 | 202.22 | O |
| ATOM | 1548 | CB | VAL | A | 234 | −54.188 | −46.023 | 87.261 | 1.00 | 202.16 | C |
| ATOM | 1549 | CG1 | VAL | A | 234 | −55.564 | −46.425 | 86.704 | 1.00 | 201.42 | C |
| ATOM | 1550 | CG2 | VAL | A | 234 | −53.117 | −46.802 | 86.575 | 1.00 | 201.67 | C |
| ATOM | 1551 | N | ASN | A | 235 | −55.179 | −43.572 | 85.322 | 1.00 | 202.73 | N |
| ATOM | 1552 | CA | ASN | A | 235 | −56.331 | −43.087 | 84.580 | 1.00 | 202.81 | C |
| ATOM | 1553 | C | ASN | A | 235 | −56.895 | −41.815 | 85.168 | 1.00 | 202.90 | C |
| ATOM | 1554 | O | ASN | A | 235 | −57.769 | −41.180 | 84.576 | 1.00 | 202.82 | O |
| ATOM | 1555 | CB | ASN | A | 235 | −57.403 | −44.168 | 84.512 | 1.00 | 202.79 | C |
| ATOM | 1556 | CG | ASN | A | 235 | −58.218 | −44.089 | 83.251 | 1.00 | 202.90 | C |
| ATOM | 1557 | OD1 | ASN | A | 235 | −57.693 | −43.836 | 82.163 | 1.00 | 202.47 | O |
| ATOM | 1558 | ND2 | ASN | A | 235 | −59.515 | −44.315 | 83.385 | 1.00 | 203.78 | N |
| ATOM | 1559 | N | GLY | A | 236 | −56.350 | −41.446 | 86.323 | 1.00 | 203.07 | N |
| ATOM | 1560 | CA | GLY | A | 236 | −56.849 | −40.352 | 87.125 | 1.00 | 203.48 | C |
| ATOM | 1561 | C | GLY | A | 236 | −57.751 | −40.889 | 88.209 | 1.00 | 203.71 | C |
| ATOM | 1562 | O | GLY | A | 236 | −58.665 | −40.195 | 88.668 | 1.00 | 203.76 | O |
| ATOM | 1563 | N | TYR | A | 237 | −57.501 | −42.126 | 88.625 | 1.00 | 203.95 | N |
| ATOM | 1564 | CA | TYR | A | 237 | −58.375 | −42.807 | 89.572 | 1.00 | 204.42 | C |
| ATOM | 1565 | C | TYR | A | 237 | −57.618 | −43.205 | 90.834 | 1.00 | 204.76 | C |
| ATOM | 1566 | O | TYR | A | 237 | −56.913 | −44.214 | 90.855 | 1.00 | 204.95 | O |
| ATOM | 1567 | CB | TYR | A | 237 | −59.007 | −44.042 | 88.926 | 1.00 | 204.18 | C |
| ATOM | 1568 | CG | TYR | A | 237 | −60.257 | −43.745 | 88.130 | 1.00 | 203.80 | C |
| ATOM | 1569 | CD1 | TYR | A | 237 | −61.517 | −43.937 | 88.681 | 1.00 | 203.24 | C |
| ATOM | 1570 | CD2 | TYR | A | 237 | −60.177 | −43.271 | 86.827 | 1.00 | 203.32 | C |
| ATOM | 1571 | CE1 | TYR | A | 237 | −62.663 | −43.666 | 87.956 | 1.00 | 202.72 | C |
| ATOM | 1572 | CE2 | TYR | A | 237 | −61.317 | −42.998 | 86.095 | 1.00 | 202.96 | C |
| ATOM | 1573 | CZ | TYR | A | 237 | −62.557 | −43.197 | 86.664 | 1.00 | 202.86 | C |
| ATOM | 1574 | OH | TYR | A | 237 | −63.695 | −42.926 | 85.939 | 1.00 | 203.14 | O |
| ATOM | 1575 | N | VAL | A | 238 | −57.768 | −42.405 | 91.885 | 1.00 | 205.06 | N |
| ATOM | 1576 | CA | VAL | A | 238 | −56.681 | −42.167 | 92.827 | 1.00 | 205.40 | C |
| ATOM | 1577 | C | VAL | A | 238 | −56.530 | −43.331 | 93.801 | 1.00 | 205.57 | C |
| ATOM | 1578 | O | VAL | A | 238 | −57.325 | −44.271 | 93.788 | 1.00 | 205.42 | O |
| ATOM | 1579 | CB | VAL | A | 238 | −56.901 | −40.868 | 93.626 | 1.00 | 205.54 | C |
| ATOM | 1580 | CG1 | VAL | A | 238 | −55.575 | −40.334 | 94.146 | 1.00 | 205.62 | C |
| ATOM | 1581 | CG2 | VAL | A | 238 | −57.602 | −39.829 | 92.764 | 1.00 | 205.51 | C |
| ATOM | 1582 | N | ASN | A | 239 | −55.504 | −43.262 | 94.643 | 1.00 | 189.08 | N |
| ATOM | 1583 | CA | ASN | A | 239 | −55.549 | −43.903 | 95.980 | 1.00 | 189.98 | C |
| ATOM | 1584 | C | ASN | A | 239 | −55.888 | −45.401 | 95.988 | 1.00 | 189.24 | C |
| ATOM | 1585 | O | ASN | A | 239 | −55.053 | −46.243 | 96.347 | 1.00 | 189.29 | O |
| ATOM | 1586 | CB | ASN | A | 239 | −56.529 | −43.104 | 96.880 | 1.00 | 190.92 | C |
| ATOM | 1587 | CG | ASN | A | 239 | −56.353 | −43.376 | 98.368 | 1.00 | 194.67 | C |
| ATOM | 1588 | OD1 | ASN | A | 239 | −57.217 | −43.993 | 98.997 | 1.00 | 195.15 | O |
| ATOM | 1589 | ND2 | ASN | A | 239 | −55.249 | −42.893 | 98.943 | 1.00 | 201.47 | N |
| ATOM | 1590 | N | ARG | A | 240 | −57.171 | −45.663 | 95.688 | 1.00 | 204.76 | N |
| ATOM | 1591 | CA | ARG | A | 240 | −57.719 | −47.006 | 95.521 | 1.00 | 204.03 | C |
| ATOM | 1592 | C | ARG | A | 240 | −58.881 | −46.986 | 94.532 | 1.00 | 202.99 | C |
| ATOM | 1593 | O | ARG | A | 240 | −59.433 | −48.033 | 94.185 | 1.00 | 202.54 | O |
| ATOM | 1594 | CB | ARG | A | 240 | −58.155 | −47.584 | 96.884 | 1.00 | 204.26 | C |
| ATOM | 1595 | CG | ARG | A | 240 | −58.728 | −46.558 | 97.883 | 1.00 | 204.78 | C |
| ATOM | 1596 | CD | ARG | A | 240 | −58.661 | −47.043 | 99.340 | 1.00 | 204.84 | C |
| ATOM | 1597 | NE | ARG | A | 240 | −58.592 | −45.912 | 100.275 | 1.00 | 206.70 | N |
| ATOM | 1598 | CZ | ARG | A | 240 | −58.733 | −45.991 | 101.600 | 1.00 | 207.34 | C |
| ATOM | 1599 | NH1 | ARG | A | 240 | −58.966 | −47.157 | 102.199 | 1.00 | 207.67 | N |
| ATOM | 1600 | NH2 | ARG | A | 240 | −58.644 | −44.888 | 102.336 | 1.00 | 207.50 | N |
| ATOM | 1601 | N | SER | A | 241 | −59.223 | −45.781 | 94.075 | 1.00 | 202.12 | N |
| ATOM | 1602 | CA | SER | A | 241 | −60.369 | −45.550 | 93.201 | 1.00 | 201.47 | C |
| ATOM | 1603 | C | SER | A | 241 | −60.462 | −46.597 | 92.118 | 1.00 | 200.98 | C |
| ATOM | 1604 | O | SER | A | 241 | −59.467 | −47.206 | 91.736 | 1.00 | 200.98 | O |
| ATOM | 1605 | CB | SER | A | 241 | −60.338 | −44.146 | 92.577 | 1.00 | 201.44 | C |
| ATOM | 1606 | OG | SER | A | 241 | −61.322 | −43.289 | 93.143 | 1.00 | 201.49 | O |
| ATOM | 1607 | N | LEU | A | 242 | −61.678 | −46.805 | 91.640 | 1.00 | 200.47 | N |
| ATOM | 1608 | CA | LEU | A | 242 | −61.941 | −47.778 | 90.601 | 1.00 | 200.00 | C |
| ATOM | 1609 | C | LEU | A | 242 | −62.684 | −47.069 | 89.463 | 1.00 | 199.64 | C |
| ATOM | 1610 | O | LEU | A | 242 | −63.489 | −46.182 | 89.724 | 1.00 | 199.70 | O |
| ATOM | 1611 | CB | LEU | A | 242 | −62.763 | −48.945 | 91.167 | 1.00 | 199.99 | C |
| ATOM | 1612 | CG | LEU | A | 242 | −62.405 | −49.688 | 92.474 | 1.00 | 199.59 | C |
| ATOM | 1613 | CD1 | LEU | A | 242 | −60.897 | −49.894 | 92.695 | 1.00 | 198.51 | C |
| ATOM | 1614 | CD2 | LEU | A | 242 | −63.056 | −49.059 | 93.704 | 1.00 | 199.37 | C |
| ATOM | 1615 | N | PRO | A | 243 | −62.377 | −47.411 | 88.199 | 1.00 | 199.23 | N |
| ATOM | 1616 | CA | PRO | A | 243 | −63.041 | −46.819 | 87.043 | 1.00 | 198.92 | C |
| ATOM | 1617 | C | PRO | A | 243 | −64.361 | −47.474 | 86.604 | 1.00 | 198.56 | C |
| ATOM | 1618 | O | PRO | A | 243 | −65.416 | −47.098 | 87.088 | 1.00 | 198.20 | O |
| ATOM | 1619 | CB | PRO | A | 243 | −61.972 | −46.918 | 85.930 | 1.00 | 199.05 | C |
| ATOM | 1620 | CG | PRO | A | 243 | −60.761 | −47.577 | 86.567 | 1.00 | 199.18 | C |
| ATOM | 1621 | CD | PRO | A | 243 | −61.300 | −48.307 | 87.768 | 1.00 | 199.34 | C |
| ATOM | 1622 | N | GLY | A | 244 | −64.309 | −48.425 | 85.681 | 1.00 | 198.50 | N |
| ATOM | 1623 | CA | GLY | A | 244 | −65.530 | −48.872 | 85.026 | 1.00 | 198.60 | C |
| ATOM | 1624 | C | GLY | A | 244 | −65.639 | −50.333 | 84.630 | 1.00 | 198.74 | C |
| ATOM | 1625 | O | GLY | A | 244 | −66.042 | −51.174 | 85.446 | 1.00 | 199.00 | O |
| ATOM | 1626 | N | LEU | A | 245 | −65.272 | −50.637 | 83.380 | 1.00 | 198.60 | N |
| ATOM | 1627 | CA | LEU | A | 245 | −65.731 | −51.870 | 82.700 | 1.00 | 198.22 | C |
| ATOM | 1628 | C | LEU | A | 245 | −64.730 | −53.037 | 82.545 | 1.00 | 197.59 | C |
| ATOM | 1629 | O | LEU | A | 245 | −63.537 | −52.946 | 82.871 | 1.00 | 196.84 | O |
| ATOM | 1630 | CB | LEU | A | 245 | −66.407 | −51.540 | 81.326 | 1.00 | 198.59 | C |
| ATOM | 1631 | CG | LEU | A | 245 | −67.833 | −50.955 | 81.109 | 1.00 | 198.67 | C |
| ATOM | 1632 | CD1 | LEU | A | 245 | −68.036 | −50.419 | 79.670 | 1.00 | 198.05 | C |
| ATOM | 1633 | CD2 | LEU | A | 245 | −68.960 | −51.950 | 81.467 | 1.00 | 198.60 | C |
| ATOM | 1634 | N | ILE | A | 246 | −65.283 | −54.118 | 82.006 | 1.00 | 197.33 | N |
| ATOM | 1635 | CA | ILE | A | 246 | −64.650 | −55.412 | 81.875 | 1.00 | 197.40 | C |
| ATOM | 1636 | C | ILE | A | 246 | −64.766 | −55.948 | 80.435 | 1.00 | 197.58 | C |
| ATOM | 1637 | O | ILE | A | 246 | −65.507 | −55.381 | 79.631 | 1.00 | 198.01 | O |
| ATOM | 1638 | CB | ILE | A | 246 | −65.345 | −56.410 | 82.815 | 1.00 | 197.31 | C |
| ATOM | 1639 | CG1 | ILE | A | 246 | −64.857 | −57.833 | 82.546 | 1.00 | 197.27 | C |
| ATOM | 1640 | CG2 | ILE | A | 246 | −66.865 | −56.333 | 82.652 | 1.00 | 196.97 | C |
| ATOM | 1641 | CD1 | ILE | A | 246 | −63.333 | −58.020 | 82.691 | 1.00 | 197.53 | C |
| ATOM | 1642 | N | GLY | A | 247 | −64.038 | −57.034 | 80.128 | 1.00 | 197.43 | N |
| ATOM | 1643 | CA | GLY | A | 247 | −64.151 | −57.788 | 78.865 | 1.00 | 197.12 | C |
| ATOM | 1644 | C | GLY | A | 247 | −64.522 | −59.256 | 79.067 | 1.00 | 197.02 | C |
| ATOM | 1645 | O | GLY | A | 247 | −64.247 | −59.831 | 80.120 | 1.00 | 196.96 | O |
| ATOM | 1646 | N | CYS | A | 248 | −65.142 | −59.856 | 78.048 | 1.00 | 196.96 | N |
| ATOM | 1647 | CA | CYS | A | 248 | −65.626 | −61.256 | 78.070 | 1.00 | 197.01 | C |
| ATOM | 1648 | C | CYS | A | 248 | −64.548 | −62.278 | 78.448 | 1.00 | 196.11 | C |
| ATOM | 1649 | O | CYS | A | 248 | −63.368 | −62.017 | 78.274 | 1.00 | 196.02 | O |
| ATOM | 1650 | CB | CYS | A | 248 | −66.269 | −61.622 | 76.721 | 1.00 | 197.58 | C |
| ATOM | 1651 | SG | CYS | A | 248 | −68.091 | −61.966 | 76.717 | 1.00 | 200.71 | S |
| ATOM | 1652 | N | HIS | A | 249 | −64.957 | −63.445 | 78.942 | 1.00 | 195.35 | N |
| ATOM | 1653 | CA | HIS | A | 249 | −64.028 | −64.328 | 79.657 | 1.00 | 194.80 | C |
| ATOM | 1654 | C | HIS | A | 249 | −63.208 | −65.316 | 78.833 | 1.00 | 194.36 | C |
| ATOM | 1655 | O | HIS | A | 249 | −61.997 | −65.430 | 79.034 | 1.00 | 194.28 | O |
| ATOM | 1656 | CB | HIS | A | 249 | −64.700 | −65.008 | 80.872 | 1.00 | 194.91 | C |
| ATOM | 1657 | CG | HIS | A | 249 | −65.368 | −66.318 | 80.577 | 1.00 | 194.89 | C |
| ATOM | 1658 | ND1 | HIS | A | 249 | −66.713 | −66.421 | 80.284 | 1.00 | 195.18 | N |
| ATOM | 1659 | CD2 | HIS | A | 249 | −64.888 | −67.584 | 80.589 | 1.00 | 194.59 | C |
| ATOM | 1660 | CE1 | HIS | A | 249 | −67.026 | −67.690 | 80.098 | 1.00 | 195.11 | C |
| ATOM | 1661 | NE2 | HIS | A | 249 | −65.937 | −68.417 | 80.280 | 1.00 | 195.01 | N |
| ATOM | 1662 | N | ARG | A | 250 | −63.857 | −66.017 | 77.910 | 1.00 | 193.82 | N |
| ATOM | 1663 | CA | ARG | A | 250 | −63.209 | −67.121 | 77.194 | 1.00 | 193.32 | C |
| ATOM | 1664 | C | ARG | A | 250 | −62.211 | −66.711 | 76.103 | 1.00 | 192.75 | C |
| ATOM | 1665 | O | ARG | A | 250 | −61.712 | −67.567 | 75.371 | 1.00 | 192.73 | O |
| ATOM | 1666 | CB | ARG | A | 250 | −64.255 | −68.071 | 76.602 | 1.00 | 193.45 | C |
| ATOM | 1667 | CG | ARG | A | 250 | −64.760 | −69.128 | 77.542 | 1.00 | 193.58 | C |
| ATOM | 1668 | CD | ARG | A | 250 | −63.808 | −70.290 | 77.686 | 1.00 | 194.06 | C |
| ATOM | 1669 | NE | ARG | A | 250 | −64.574 | −71.513 | 77.915 | 1.00 | 195.83 | N |
| ATOM | 1670 | CZ | ARG | A | 250 | −64.999 | −71.941 | 79.106 | 1.00 | 196.72 | C |
| ATOM | 1671 | NH1 | ARG | A | 250 | −64.726 | −71.256 | 80.213 | 1.00 | 197.39 | N |
| ATOM | 1672 | NH2 | ARG | A | 250 | −65.698 | −73.068 | 79.195 | 1.00 | 196.47 | N |
| ATOM | 1673 | N | LYS | A | 251 | −61.914 | −65.422 | 75.983 | 1.00 | 191.96 | N |
| ATOM | 1674 | CA | LYS | A | 251 | −60.928 | −64.993 | 74.995 | 1.00 | 191.32 | C |
| ATOM | 1675 | C | LYS | A | 251 | −60.044 | −63.835 | 75.462 | 1.00 | 190.61 | C |
| ATOM | 1676 | O | LYS | A | 251 | −59.841 | −63.654 | 76.663 | 1.00 | 190.64 | O |
| ATOM | 1677 | CB | LYS | A | 251 | −61.570 | −64.744 | 73.612 | 1.00 | 191.63 | C |
| ATOM | 1678 | CG | LYS | A | 251 | −62.994 | −64.174 | 73.605 | 1.00 | 192.25 | C |
| ATOM | 1679 | CD | LYS | A | 251 | −62.985 | −62.664 | 73.748 | 1.00 | 193.41 | C |
| ATOM | 1680 | CE | LYS | A | 251 | −63.019 | −62.260 | 75.210 | 1.00 | 193.71 | C |
| ATOM | 1681 | NZ | LYS | A | 251 | −61.946 | −61.285 | 75.538 | 1.00 | 194.05 | N |
| ATOM | 1682 | N | SER | A | 252 | −59.533 | −63.055 | 74.511 | 1.00 | 189.68 | N |
| ATOM | 1683 | CA | SER | A | 252 | −58.449 | −62.114 | 74.771 | 1.00 | 188.78 | C |
| ATOM | 1684 | C | SER | A | 252 | −58.889 | −60.657 | 74.834 | 1.00 | 188.37 | C |
| ATOM | 1685 | O | SER | A | 252 | −59.880 | −60.270 | 74.230 | 1.00 | 188.19 | O |
| ATOM | 1686 | CB | SER | A | 252 | −57.335 | −62.306 | 73.736 | 1.00 | 188.72 | C |
| ATOM | 1687 | OG | SER | A | 252 | −57.841 | −62.843 | 72.523 | 1.00 | 188.17 | O |
| ATOM | 1688 | N | VAL | A | 253 | −58.139 | −59.856 | 75.580 | 1.00 | 188.01 | N |
| ATOM | 1689 | CA | VAL | A | 253 | −58.448 | −58.448 | 75.752 | 1.00 | 187.98 | C |
| ATOM | 1690 | C | VAL | A | 253 | −57.189 | −57.608 | 75.601 | 1.00 | 188.17 | C |
| ATOM | 1691 | O | VAL | A | 253 | −56.211 | −57.817 | 76.329 | 1.00 | 188.26 | O |
| ATOM | 1692 | CB | VAL | A | 253 | −59.050 | −58.190 | 77.132 | 1.00 | 187.95 | C |
| ATOM | 1693 | CG1 | VAL | A | 253 | −59.015 | −56.702 | 77.475 | 1.00 | 187.84 | C |
| ATOM | 1694 | CG2 | VAL | A | 253 | −60.467 | −58.721 | 77.194 | 1.00 | 188.25 | C |
| ATOM | 1695 | N | TYR | A | 254 | −57.237 | −56.647 | 74.675 | 1.00 | 188.28 | N |
| ATOM | 1696 | CA | TYR | A | 254 | −56.085 | −55.808 | 74.306 | 1.00 | 188.34 | C |
| ATOM | 1697 | C | TYR | A | 254 | −56.147 | −54.439 | 74.939 | 1.00 | 188.63 | C |
| ATOM | 1698 | O | TYR | A | 254 | −57.202 | −53.801 | 74.911 | 1.00 | 188.61 | O |
| ATOM | 1699 | CB | TYR | A | 254 | −56.056 | −55.603 | 72.796 | 1.00 | 188.20 | C |
| ATOM | 1700 | CG | TYR | A | 254 | −55.869 | −56.871 | 72.027 | 1.00 | 188.01 | C |
| ATOM | 1701 | CD1 | TYR | A | 254 | −54.621 | −57.212 | 71.531 | 1.00 | 187.87 | C |
| ATOM | 1702 | CD2 | TYR | A | 254 | −56.934 | −57.739 | 71.801 | 1.00 | 187.63 | C |
| ATOM | 1703 | CE1 | TYR | A | 254 | −54.430 | −58.374 | 70.832 | 1.00 | 187.77 | C |
| ATOM | 1704 | CE2 | TYR | A | 254 | −56.756 | −58.908 | 71.103 | 1.00 | 187.55 | C |
| ATOM | 1705 | CZ | TYR | A | 254 | −55.497 | −59.219 | 70.622 | 1.00 | 187.88 | C |
| ATOM | 1706 | OH | TYR | A | 254 | −55.291 | −60.379 | 69.918 | 1.00 | 188.62 | O |
| ATOM | 1707 | N | TRP | A | 255 | −55.027 | −53.973 | 75.496 | 1.00 | 189.05 | N |
| ATOM | 1708 | CA | TRP | A | 255 | −54.982 | −52.598 | 76.015 | 1.00 | 189.54 | C |
| ATOM | 1709 | C | TRP | A | 255 | −53.805 | −51.780 | 75.507 | 1.00 | 189.75 | C |
| ATOM | 1710 | O | TRP | A | 255 | −52.644 | −52.174 | 75.668 | 1.00 | 189.64 | O |
| ATOM | 1711 | CB | TRP | A | 255 | −54.987 | −52.497 | 77.558 | 1.00 | 189.77 | C |
| ATOM | 1712 | CG | TRP | A | 255 | −55.547 | −53.645 | 78.412 | 1.00 | 189.70 | C |
| ATOM | 1713 | CD1 | TRP | A | 255 | −56.848 | −53.834 | 78.840 | 1.00 | 189.16 | C |
| ATOM | 1714 | CD2 | TRP | A | 255 | −54.785 | −54.686 | 78.996 | 1.00 | 188.95 | C |
| ATOM | 1715 | NE1 | TRP | A | 255 | −56.927 | −54.956 | 79.618 | 1.00 | 187.80 | N |
| ATOM | 1716 | CE2 | TRP | A | 255 | −55.674 | −55.496 | 79.729 | 1.00 | 188.41 | C |
| ATOM | 1717 | CE3 | TRP | A | 255 | −53.428 | −55.029 | 78.951 | 1.00 | 189.34 | C |
| ATOM | 1718 | CZ2 | TRP | A | 255 | −55.248 | −56.622 | 80.410 | 1.00 | 189.51 | C |
| ATOM | 1719 | CZ3 | TRP | A | 255 | −53.003 | −56.146 | 79.636 | 1.00 | 189.50 | C |
| ATOM | 1720 | CH2 | TRP | A | 255 | −53.907 | −56.930 | 80.358 | 1.00 | 189.80 | C |
| ATOM | 1721 | N | HIS | A | 256 | −54.127 | −50.627 | 74.920 | 1.00 | 190.24 | N |
| ATOM | 1722 | CA | HIS | A | 256 | −53.127 | −49.619 | 74.552 | 1.00 | 190.66 | C |
| ATOM | 1723 | C | HIS | A | 256 | −52.797 | −48.838 | 75.821 | 1.00 | 190.81 | C |
| ATOM | 1724 | O | HIS | A | 256 | −53.637 | −48.144 | 76.405 | 1.00 | 190.73 | O |
| ATOM | 1725 | CB | HIS | A | 256 | −53.609 | −48.693 | 73.410 | 1.00 | 190.66 | C |
| ATOM | 1726 | CG | HIS | A | 256 | −54.344 | −49.408 | 72.318 | 1.00 | 190.45 | C |
| ATOM | 1727 | ND1 | HIS | A | 256 | −53.700 | −50.051 | 71.284 | 1.00 | 190.67 | N |
| ATOM | 1728 | CD2 | HIS | A | 256 | −55.670 | −49.601 | 72.113 | 1.00 | 190.02 | C |
| ATOM | 1729 | CE1 | HIS | A | 256 | −54.598 | −50.610 | 70.490 | 1.00 | 190.87 | C |
| ATOM | 1730 | NE2 | HIS | A | 256 | −55.801 | −50.349 | 70.969 | 1.00 | 190.03 | N |
| ATOM | 1731 | N | VAL | A | 257 | −51.565 | −48.986 | 76.260 | 1.00 | 190.98 | N |
| ATOM | 1732 | CA | VAL | A | 257 | −51.200 | −48.524 | 77.564 | 1.00 | 191.30 | C |
| ATOM | 1733 | C | VAL | A | 257 | −50.232 | −47.383 | 77.365 | 1.00 | 191.50 | C |
| ATOM | 1734 | O | VAL | A | 257 | −49.188 | −47.548 | 76.739 | 1.00 | 191.55 | O |
| ATOM | 1735 | CB | VAL | A | 257 | −50.666 | −49.720 | 78.423 | 1.00 | 191.43 | C |
| ATOM | 1736 | CG1 | VAL | A | 257 | −49.906 | −50.745 | 77.567 | 1.00 | 191.85 | C |
| ATOM | 1737 | CG2 | VAL | A | 257 | −49.840 | −49.267 | 79.609 | 1.00 | 191.63 | C |
| ATOM | 1738 | N | ILE | A | 258 | −50.621 | −46.211 | 77.854 | 1.00 | 192.05 | N |
| ATOM | 1739 | CA | ILE | A | 258 | −49.834 | −44.980 | 77.699 | 1.00 | 192.92 | C |
| ATOM | 1740 | C | ILE | A | 258 | −49.149 | −44.551 | 79.010 | 1.00 | 193.85 | C |
| ATOM | 1741 | O | ILE | A | 258 | −49.831 | −44.158 | 79.974 | 1.00 | 194.30 | O |
| ATOM | 1742 | CB | ILE | A | 258 | −50.716 | −43.802 | 77.171 | 1.00 | 192.68 | C |
| ATOM | 1743 | CG1 | ILE | A | 258 | −51.419 | −44.200 | 75.869 | 1.00 | 192.79 | C |
| ATOM | 1744 | CG2 | ILE | A | 258 | −49.888 | −42.510 | 77.005 | 1.00 | 191.94 | C |
| ATOM | 1745 | CD1 | ILE | A | 258 | −52.623 | −43.347 | 75.506 | 1.00 | 192.94 | C |
| ATOM | 1746 | N | GLY | A | 259 | −47.813 | −44.620 | 79.042 | 1.00 | 194.53 | N |
| ATOM | 1747 | CA | GLY | A | 259 | −47.017 | −44.148 | 80.197 | 1.00 | 195.25 | C |
| ATOM | 1748 | C | GLY | A | 259 | −46.955 | −42.628 | 80.320 | 1.00 | 195.76 | C |
| ATOM | 1749 | O | GLY | A | 259 | −46.876 | −41.921 | 79.301 | 1.00 | 195.90 | O |
| ATOM | 1750 | N | MET | A | 260 | −46.978 | −42.120 | 81.562 | 1.00 | 196.03 | N |
| ATOM | 1751 | CA | MET | A | 260 | −47.095 | −40.665 | 81.808 | 1.00 | 196.23 | C |
| ATOM | 1752 | C | MET | A | 260 | −46.229 | −40.080 | 82.941 | 1.00 | 195.44 | C |
| ATOM | 1753 | O | MET | A | 260 | −45.615 | −40.811 | 83.725 | 1.00 | 195.44 | O |
| ATOM | 1754 | CB | MET | A | 260 | −48.570 | −40.274 | 82.013 | 1.00 | 196.72 | C |
| ATOM | 1755 | CG | MET | A | 260 | −48.929 | −38.828 | 81.634 | 1.00 | 199.11 | C |
| ATOM | 1756 | SD | MET | A | 260 | −49.113 | −38.512 | 79.842 | 1.00 | 204.91 | S |
| ATOM | 1757 | CE | MET | A | 260 | −47.409 | −38.439 | 79.237 | 1.00 | 203.43 | C |
| ATOM | 1758 | N | GLY | A | 261 | −46.183 | −38.751 | 82.993 | 1.00 | 194.61 | N |
| ATOM | 1759 | CA | GLY | A | 261 | −45.514 | −38.046 | 84.060 | 1.00 | 193.75 | C |
| ATOM | 1760 | C | GLY | A | 261 | −44.801 | −36.785 | 83.640 | 1.00 | 193.18 | C |
| ATOM | 1761 | O | GLY | A | 261 | −44.836 | −36.379 | 82.486 | 1.00 | 193.16 | O |
| ATOM | 1762 | N | THR | A | 262 | −44.148 | −36.173 | 84.613 | 1.00 | 192.73 | N |
| ATOM | 1763 | CA | THR | A | 262 | −43.374 | −34.977 | 84.411 | 1.00 | 192.41 | C |
| ATOM | 1764 | C | THR | A | 262 | −42.019 | −35.180 | 85.078 | 1.00 | 192.28 | C |
| ATOM | 1765 | O | THR | A | 262 | −41.285 | −34.233 | 85.323 | 1.00 | 192.10 | O |
| ATOM | 1766 | CB | THR | A | 262 | −44.112 | −33.764 | 84.998 | 1.00 | 192.39 | C |
| ATOM | 1767 | OG1 | THR | A | 262 | −43.643 | −32.562 | 84.385 | 1.00 | 192.50 | O |
| ATOM | 1768 | CG2 | THR | A | 262 | −43.940 | −33.676 | 86.520 | 1.00 | 192.68 | C |
| ATOM | 1769 | N | THR | A | 263 | −41.700 | −36.437 | 85.368 | 1.00 | 192.46 | N |
| ATOM | 1770 | CA | THR | A | 263 | −40.441 | −36.816 | 86.009 | 1.00 | 192.78 | C |
| ATOM | 1771 | C | THR | A | 263 | −40.088 | −38.285 | 85.721 | 1.00 | 193.14 | C |
| ATOM | 1772 | O | THR | A | 263 | −40.973 | −39.095 | 85.429 | 1.00 | 193.25 | O |
| ATOM | 1773 | CB | THR | A | 263 | −40.465 | −36.547 | 87.548 | 1.00 | 192.74 | C |
| ATOM | 1774 | OG1 | THR | A | 263 | −39.196 | −36.872 | 88.126 | 1.00 | 192.65 | O |
| ATOM | 1775 | CG2 | THR | A | 263 | −41.545 | −37.361 | 88.243 | 1.00 | 192.82 | C |
| ATOM | 1776 | N | PRO | A | 264 | −38.785 | −38.620 | 85.743 | 1.00 | 193.52 | N |
| ATOM | 1777 | CA | PRO | A | 264 | −38.308 | −40.015 | 85.764 | 1.00 | 193.92 | C |
| ATOM | 1778 | C | PRO | A | 264 | −38.875 | −40.910 | 86.904 | 1.00 | 194.13 | C |
| ATOM | 1779 | O | PRO | A | 264 | −38.159 | −41.228 | 87.867 | 1.00 | 194.27 | O |
| ATOM | 1780 | CB | PRO | A | 264 | −36.779 | −39.852 | 85.905 | 1.00 | 194.10 | C |
| ATOM | 1781 | CG | PRO | A | 264 | −36.544 | −38.398 | 86.275 | 1.00 | 193.67 | C |
| ATOM | 1782 | CD | PRO | A | 264 | −37.666 | −37.663 | 85.650 | 1.00 | 193.45 | C |
| ATOM | 1783 | N | GLU | A | 265 | −40.130 | −41.343 | 86.774 | 1.00 | 194.08 | N |
| ATOM | 1784 | CA | GLU | A | 265 | −40.814 | −42.022 | 87.876 | 1.00 | 194.35 | C |
| ATOM | 1785 | C | GLU | A | 265 | −41.389 | −43.380 | 87.480 | 1.00 | 193.74 | C |
| ATOM | 1786 | O | GLU | A | 265 | −42.573 | −43.483 | 87.142 | 1.00 | 193.96 | O |
| ATOM | 1787 | CB | GLU | A | 265 | −41.912 | −41.118 | 88.439 | 1.00 | 194.30 | C |
| ATOM | 1788 | CG | GLU | A | 265 | −42.306 | −41.421 | 89.882 | 1.00 | 195.50 | C |
| ATOM | 1789 | CD | GLU | A | 265 | −42.968 | −40.226 | 90.594 | 1.00 | 195.85 | C |
| ATOM | 1790 | OE1 | GLU | A | 265 | −43.412 | −39.267 | 89.904 | 1.00 | 197.29 | O |
| ATOM | 1791 | OE2 | GLU | A | 265 | −43.046 | −40.254 | 91.852 | 1.00 | 197.60 | O |
| ATOM | 1792 | N | VAL | A | 266 | −40.547 | −44.412 | 87.562 | 1.00 | 193.00 | N |
| ATOM | 1793 | CA | VAL | A | 266 | −40.850 | −45.769 | 87.076 | 1.00 | 191.97 | C |
| ATOM | 1794 | C | VAL | A | 266 | −41.887 | −46.525 | 87.888 | 1.00 | 191.40 | C |
| ATOM | 1795 | O | VAL | A | 266 | −41.949 | −46.378 | 89.104 | 1.00 | 191.31 | O |
| ATOM | 1796 | CB | VAL | A | 266 | −39.582 | −46.624 | 87.027 | 1.00 | 191.93 | C |
| ATOM | 1797 | CG1 | VAL | A | 266 | −38.759 | −46.248 | 85.814 | 1.00 | 192.14 | C |
| ATOM | 1798 | CG2 | VAL | A | 266 | −38.767 | −46.458 | 88.315 | 1.00 | 191.73 | C |
| ATOM | 1799 | N | HIS | A | 267 | −42.690 | −47.333 | 87.195 | 1.00 | 190.86 | N |
| ATOM | 1800 | CA | HIS | A | 267 | −43.689 | −48.229 | 87.816 | 1.00 | 190.49 | C |
| ATOM | 1801 | C | HIS | A | 267 | −43.441 | −49.703 | 87.433 | 1.00 | 189.94 | C |
| ATOM | 1802 | O | HIS | A | 267 | −42.401 | −50.020 | 86.858 | 1.00 | 190.36 | O |
| ATOM | 1803 | CB | HIS | A | 267 | −45.116 | −47.816 | 87.432 | 1.00 | 190.53 | C |
| ATOM | 1804 | CG | HIS | A | 267 | −45.472 | −46.423 | 87.837 | 1.00 | 191.04 | C |
| ATOM | 1805 | ND1 | HIS | A | 267 | −46.212 | −46.140 | 88.965 | 1.00 | 191.44 | N |
| ATOM | 1806 | CD2 | HIS | A | 267 | −45.184 | −45.231 | 87.265 | 1.00 | 191.70 | C |
| ATOM | 1807 | CE1 | HIS | A | 267 | −46.362 | −44.831 | 89.070 | 1.00 | 192.13 | C |
| ATOM | 1808 | NE2 | HIS | A | 267 | −45.747 | −44.257 | 88.052 | 1.00 | 192.19 | N |
| ATOM | 1809 | N | SER | A | 268 | −44.383 | −50.592 | 87.752 | 1.00 | 188.87 | N |
| ATOM | 1810 | CA | SER | A | 268 | −44.286 | −52.013 | 87.415 | 1.00 | 187.81 | C |
| ATOM | 1811 | C | SER | A | 268 | −45.643 | −52.633 | 87.696 | 1.00 | 187.54 | C |
| ATOM | 1812 | O | SER | A | 268 | −46.002 | −52.862 | 88.854 | 1.00 | 187.70 | O |
| ATOM | 1813 | CB | SER | A | 268 | −43.200 | −52.702 | 88.246 | 1.00 | 187.61 | C |
| ATOM | 1814 | OG | SER | A | 268 | −42.979 | −54.022 | 87.797 | 1.00 | 186.77 | O |
| ATOM | 1815 | N | ILE | A | 269 | −46.398 | −52.900 | 86.634 | 1.00 | 186.94 | N |
| ATOM | 1816 | CA | ILE | A | 269 | −47.841 | −53.170 | 86.738 | 1.00 | 186.30 | C |
| ATOM | 1817 | C | ILE | A | 269 | −48.226 | −54.648 | 86.571 | 1.00 | 186.26 | C |
| ATOM | 1818 | O | ILE | A | 269 | −47.618 | −55.363 | 85.782 | 1.00 | 186.45 | O |
| ATOM | 1819 | CB | ILE | A | 269 | −48.611 | −52.258 | 85.753 | 1.00 | 185.99 | C |
| ATOM | 1820 | CG1 | ILE | A | 269 | −48.727 | −50.852 | 86.339 | 1.00 | 185.73 | C |
| ATOM | 1821 | CG2 | ILE | A | 269 | −49.990 | −52.796 | 85.443 | 1.00 | 185.38 | C |
| ATOM | 1822 | CD1 | ILE | A | 269 | −47.466 | −50.018 | 86.293 | 1.00 | 185.46 | C |
| ATOM | 1823 | N | PHE | A | 270 | −49.223 | −55.110 | 87.325 | 1.00 | 186.09 | N |
| ATOM | 1824 | CA | PHE | A | 270 | −49.615 | −56.517 | 87.265 | 1.00 | 186.06 | C |
| ATOM | 1825 | C | PHE | A | 270 | −51.127 | −56.736 | 87.300 | 1.00 | 186.24 | C |
| ATOM | 1826 | O | PHE | A | 270 | −51.898 | −55.849 | 87.671 | 1.00 | 185.97 | O |
| ATOM | 1827 | CB | PHE | A | 270 | −49.040 | −57.305 | 88.452 | 1.00 | 186.09 | C |
| ATOM | 1828 | CG | PHE | A | 270 | −47.548 | −57.180 | 88.658 | 1.00 | 185.85 | C |
| ATOM | 1829 | CD1 | PHE | A | 270 | −46.763 | −58.324 | 88.732 | 1.00 | 185.88 | C |
| ATOM | 1830 | CD2 | PHE | A | 270 | −46.940 | −55.942 | 88.862 | 1.00 | 185.31 | C |
| ATOM | 1831 | CE1 | PHE | A | 270 | −45.391 | −58.237 | 88.955 | 1.00 | 185.71 | C |
| ATOM | 1832 | CE2 | PHE | A | 270 | −45.571 | −55.843 | 89.074 | 1.00 | 185.19 | C |
| ATOM | 1833 | CZ | PHE | A | 270 | −44.796 | −56.990 | 89.125 | 1.00 | 185.58 | C |
| ATOM | 1834 | N | LEU | A | 271 | −51.514 | −57.940 | 86.885 | 1.00 | 186.64 | N |
| ATOM | 1835 | CA | LEU | A | 271 | −52.728 | −58.645 | 87.332 | 1.00 | 187.27 | C |
| ATOM | 1836 | C | LEU | A | 271 | −52.672 | −60.105 | 86.824 | 1.00 | 187.96 | C |
| ATOM | 1837 | O | LEU | A | 271 | −51.710 | −60.479 | 86.132 | 1.00 | 188.39 | O |
| ATOM | 1838 | CB | LEU | A | 271 | −54.024 | −57.933 | 86.944 | 1.00 | 186.94 | C |
| ATOM | 1839 | CG | LEU | A | 271 | −54.462 | −57.853 | 85.489 | 1.00 | 186.80 | C |
| ATOM | 1840 | CD1 | LEU | A | 271 | −55.196 | −59.112 | 85.013 | 1.00 | 186.15 | C |
| ATOM | 1841 | CD2 | LEU | A | 271 | −55.359 | −56.659 | 85.360 | 1.00 | 186.95 | C |
| ATOM | 1842 | N | GLU | A | 272 | −53.698 | −60.908 | 87.144 | 1.00 | 188.36 | N |
| ATOM | 1843 | CA | GLU | A | 272 | −53.626 | −62.391 | 87.072 | 1.00 | 188.44 | C |
| ATOM | 1844 | C | GLU | A | 272 | −54.000 | −63.052 | 85.736 | 1.00 | 188.53 | C |
| ATOM | 1845 | O | GLU | A | 272 | −55.117 | −62.919 | 85.212 | 1.00 | 188.34 | O |
| ATOM | 1846 | CB | GLU | A | 272 | −54.379 | −63.001 | 88.248 | 1.00 | 188.38 | C |
| ATOM | 1847 | CG | GLU | A | 272 | −53.750 | −62.657 | 89.609 | 1.00 | 188.85 | C |
| ATOM | 1848 | CD | GLU | A | 272 | −53.440 | −61.166 | 89.797 | 1.00 | 189.02 | C |
| ATOM | 1849 | OE1 | GLU | A | 272 | −52.254 | −60.838 | 90.045 | 1.00 | 188.91 | O |
| ATOM | 1850 | OE2 | GLU | A | 272 | −54.370 | −60.328 | 89.673 | 1.00 | 188.59 | O |
| ATOM | 1851 | N | GLY | A | 273 | −53.039 | −63.809 | 85.228 | 1.00 | 188.75 | N |
| ATOM | 1852 | CA | GLY | A | 273 | −52.891 | −63.989 | 83.799 | 1.00 | 189.24 | C |
| ATOM | 1853 | C | GLY | A | 273 | −51.642 | −63.203 | 83.421 | 1.00 | 189.59 | C |
| ATOM | 1854 | O | GLY | A | 273 | −51.275 | −62.230 | 84.084 | 1.00 | 189.17 | O |
| ATOM | 1855 | N | HIS | A | 274 | −50.985 | −63.605 | 82.346 | 1.00 | 190.18 | N |
| ATOM | 1856 | CA | HIS | A | 274 | −49.600 | −63.229 | 82.199 | 1.00 | 190.98 | C |
| ATOM | 1857 | C | HIS | A | 274 | −49.270 | −61.877 | 81.599 | 1.00 | 191.09 | C |
| ATOM | 1858 | O | HIS | A | 274 | −48.274 | −61.304 | 82.004 | 1.00 | 191.23 | O |
| ATOM | 1859 | CB | HIS | A | 274 | −48.785 | −64.371 | 81.596 | 1.00 | 191.35 | C |
| ATOM | 1860 | CG | HIS | A | 274 | −48.250 | −65.324 | 82.631 | 1.00 | 193.95 | C |
| ATOM | 1861 | ND1 | HIS | A | 274 | −49.066 | −65.983 | 83.533 | 1.00 | 196.26 | N |
| ATOM | 1862 | CD2 | HIS | A | 274 | −46.982 | −65.713 | 82.924 | 1.00 | 195.66 | C |
| ATOM | 1863 | CE1 | HIS | A | 274 | −48.327 | −66.738 | 84.329 | 1.00 | 196.27 | C |
| ATOM | 1864 | NE2 | HIS | A | 274 | −47.058 | −66.595 | 83.979 | 1.00 | 196.19 | N |
| ATOM | 1865 | N | THR | A | 275 | −50.100 | −61.367 | 80.680 | 1.00 | 191.54 | N |
| ATOM | 1866 | CA | THR | A | 275 | −49.899 | −60.056 | 79.965 | 1.00 | 191.93 | C |
| ATOM | 1867 | C | THR | A | 275 | −48.900 | −60.193 | 78.775 | 1.00 | 192.63 | C |
| ATOM | 1868 | O | THR | A | 275 | −48.026 | −59.353 | 78.537 | 1.00 | 192.52 | O |
| ATOM | 1869 | CB | THR | A | 275 | −49.629 | −58.832 | 80.962 | 1.00 | 191.84 | C |
| ATOM | 1870 | OG1 | THR | A | 275 | −50.425 | −57.683 | 80.625 | 1.00 | 189.99 | O |
| ATOM | 1871 | CG2 | THR | A | 275 | −48.146 | −58.468 | 81.052 | 1.00 | 191.98 | C |
| ATOM | 1872 | N | PHE | A | 276 | −49.100 | −61.262 | 78.006 | 1.00 | 193.59 | N |
| ATOM | 1873 | CA | PHE | A | 276 | −48.083 | −61.811 | 77.098 | 1.00 | 194.33 | C |
| ATOM | 1874 | C | PHE | A | 276 | −47.720 | −60.956 | 75.890 | 1.00 | 194.76 | C |
| ATOM | 1875 | O | PHE | A | 276 | −46.648 | −61.157 | 75.345 | 1.00 | 194.72 | O |
| ATOM | 1876 | CB | PHE | A | 276 | −48.408 | −63.277 | 76.669 | 1.00 | 194.30 | C |
| ATOM | 1877 | CG | PHE | A | 276 | −47.625 | −64.389 | 77.446 | 1.00 | 194.85 | C |
| ATOM | 1878 | CD1 | PHE | A | 276 | −48.062 | −65.727 | 77.399 | 1.00 | 194.09 | C |
| ATOM | 1879 | CD2 | PHE | A | 276 | −46.466 | −64.103 | 78.203 | 1.00 | 194.64 | C |
| ATOM | 1880 | CE1 | PHE | A | 276 | −47.361 | −66.734 | 78.058 | 1.00 | 192.41 | C |
| ATOM | 1881 | CE2 | PHE | A | 276 | −45.766 | −65.120 | 78.877 | 1.00 | 193.27 | C |
| ATOM | 1882 | CZ | PHE | A | 276 | −46.219 | −66.428 | 78.802 | 1.00 | 192.82 | C |
| ATOM | 1883 | N | LEU | A | 277 | −48.563 | −60.019 | 75.453 | 1.00 | 195.69 | N |
| ATOM | 1884 | CA | LEU | A | 277 | −48.086 | −59.139 | 74.363 | 1.00 | 196.79 | C |
| ATOM | 1885 | C | LEU | A | 277 | −47.125 | −58.074 | 74.935 | 1.00 | 197.48 | C |
| ATOM | 1886 | O | LEU | A | 277 | −47.393 | −57.450 | 75.964 | 1.00 | 197.41 | O |
| ATOM | 1887 | CB | LEU | A | 277 | −49.219 | −58.551 | 73.439 | 1.00 | 196.85 | C |
| ATOM | 1888 | CG | LEU | A | 277 | −49.255 | −58.168 | 71.894 | 1.00 | 195.94 | C |
| ATOM | 1889 | CD1 | LEU | A | 277 | −48.132 | −57.253 | 71.365 | 1.00 | 195.01 | C |
| ATOM | 1890 | CD2 | LEU | A | 277 | −49.427 | −59.336 | 70.898 | 1.00 | 194.92 | C |
| ATOM | 1891 | N | VAL | A | 278 | −45.961 | −57.972 | 74.298 | 1.00 | 198.37 | N |
| ATOM | 1892 | CA | VAL | A | 278 | −45.114 | −56.781 | 74.314 | 1.00 | 199.42 | C |
| ATOM | 1893 | C | VAL | A | 278 | −44.734 | −56.581 | 72.819 | 1.00 | 200.00 | C |
| ATOM | 1894 | O | VAL | A | 278 | −45.201 | −55.626 | 72.160 | 1.00 | 200.50 | O |
| ATOM | 1895 | CB | VAL | A | 278 | −43.948 | −56.897 | 75.358 | 1.00 | 199.47 | C |
| ATOM | 1896 | CG1 | VAL | A | 278 | −42.624 | −56.270 | 74.865 | 1.00 | 199.91 | C |
| ATOM | 1897 | CG2 | VAL | A | 278 | −44.388 | −56.287 | 76.694 | 1.00 | 199.21 | C |
| ATOM | 1898 | N | ARG | A | 279 | −43.931 | −57.494 | 72.277 | 1.00 | 200.03 | N |
| ATOM | 1899 | CA | ARG | A | 279 | −44.138 | −57.888 | 70.907 | 1.00 | 200.11 | C |
| ATOM | 1900 | C | ARG | A | 279 | −44.947 | −59.118 | 71.242 | 1.00 | 200.03 | C |
| ATOM | 1901 | O | ARG | A | 279 | −45.573 | −59.147 | 72.284 | 1.00 | 199.70 | O |
| ATOM | 1902 | CB | ARG | A | 279 | −42.827 | −58.253 | 70.230 | 1.00 | 200.32 | C |
| ATOM | 1903 | CG | ARG | A | 279 | −42.969 | −58.461 | 68.714 | 1.00 | 201.17 | C |
| ATOM | 1904 | CD | ARG | A | 279 | −41.860 | −59.359 | 68.114 | 1.00 | 201.58 | C |
| ATOM | 1905 | NE | ARG | A | 279 | −40.557 | −58.690 | 68.053 | 1.00 | 201.55 | N |
| ATOM | 1906 | CZ | ARG | A | 279 | −40.296 | −57.588 | 67.348 | 1.00 | 200.88 | C |
| ATOM | 1907 | NH1 | ARG | A | 279 | −41.248 | −56.991 | 66.637 | 1.00 | 200.56 | N |
| ATOM | 1908 | NH2 | ARG | A | 279 | −39.077 | −57.070 | 67.367 | 1.00 | 200.13 | N |
| ATOM | 1909 | N | ASN | A | 280 | −44.936 | −60.152 | 70.421 | 1.00 | 200.41 | N |
| ATOM | 1910 | CA | ASN | A | 280 | −45.248 | −61.445 | 70.992 | 1.00 | 200.81 | C |
| ATOM | 1911 | C | ASN | A | 280 | −43.973 | −61.899 | 71.717 | 1.00 | 200.96 | C |
| ATOM | 1912 | O | ASN | A | 280 | −43.614 | −63.076 | 71.719 | 1.00 | 200.70 | O |
| ATOM | 1913 | CB | ASN | A | 280 | −45.753 | −62.439 | 69.951 | 1.00 | 200.83 | C |
| ATOM | 1914 | CG | ASN | A | 280 | −44.639 | −63.037 | 69.132 | 1.00 | 201.99 | C |
| ATOM | 1915 | OD1 | ASN | A | 280 | −43.623 | −62.386 | 68.853 | 1.00 | 203.36 | O |
| ATOM | 1916 | ND2 | ASN | A | 280 | −44.819 | −64.293 | 68.737 | 1.00 | 203.39 | N |
| ATOM | 1917 | N | HIS | A | 281 | −43.271 | −60.913 | 72.286 | 1.00 | 201.36 | N |
| ATOM | 1918 | CA | HIS | A | 281 | −42.239 | −61.138 | 73.292 | 1.00 | 201.63 | C |
| ATOM | 1919 | C | HIS | A | 281 | −42.974 | −61.064 | 74.613 | 1.00 | 201.39 | C |
| ATOM | 1920 | O | HIS | A | 281 | −43.705 | −60.096 | 74.851 | 1.00 | 201.47 | O |
| ATOM | 1921 | CB | HIS | A | 281 | −41.143 | −60.080 | 73.229 | 1.00 | 201.91 | C |
| ATOM | 1922 | CG | HIS | A | 281 | −39.783 | −60.636 | 73.497 | 1.00 | 202.85 | C |
| ATOM | 1923 | ND1 | HIS | A | 281 | −39.437 | −61.194 | 74.709 | 1.00 | 203.51 | N |
| ATOM | 1924 | CD2 | HIS | A | 281 | −38.700 | −60.769 | 72.696 | 1.00 | 203.71 | C |
| ATOM | 1925 | CE1 | HIS | A | 281 | −38.190 | −61.624 | 74.652 | 1.00 | 204.01 | C |
| ATOM | 1926 | NE2 | HIS | A | 281 | −37.720 | −61.378 | 73.442 | 1.00 | 204.37 | N |
| ATOM | 1927 | N | ARG | A | 282 | −42.779 | −62.071 | 75.466 | 1.00 | 201.00 | N |
| ATOM | 1928 | CA | ARG | A | 282 | −43.866 | −62.505 | 76.361 | 1.00 | 200.74 | C |
| ATOM | 1929 | C | ARG | A | 282 | −43.659 | −62.390 | 77.892 | 1.00 | 199.76 | C |
| ATOM | 1930 | O | ARG | A | 282 | −43.028 | −63.231 | 78.539 | 1.00 | 199.28 | O |
| ATOM | 1931 | CB | ARG | A | 282 | −44.354 | −63.884 | 75.900 | 1.00 | 201.31 | C |
| ATOM | 1932 | CG | ARG | A | 282 | −44.098 | −64.131 | 74.389 | 1.00 | 203.64 | C |
| ATOM | 1933 | CD | ARG | A | 282 | −45.299 | −64.675 | 73.582 | 1.00 | 208.47 | C |
| ATOM | 1934 | NE | ARG | A | 282 | −44.864 | −65.262 | 72.299 | 1.00 | 212.09 | N |
| ATOM | 1935 | CZ | ARG | A | 282 | −45.475 | −66.266 | 71.652 | 1.00 | 214.10 | C |
| ATOM | 1936 | NH1 | ARG | A | 282 | −46.574 | −66.843 | 72.149 | 1.00 | 214.76 | N |
| ATOM | 1937 | NH2 | ARG | A | 282 | −44.968 | −66.722 | 70.507 | 1.00 | 214.62 | N |
| ATOM | 1938 | N | GLN | A | 283 | −44.287 | −61.351 | 78.443 | 1.00 | 198.94 | N |
| ATOM | 1939 | CA | GLN | A | 283 | −43.788 | −60.653 | 79.624 | 1.00 | 198.31 | C |
| ATOM | 1940 | C | GLN | A | 283 | −44.508 | −60.875 | 80.910 | 1.00 | 197.75 | C |
| ATOM | 1941 | O | GLN | A | 283 | −45.725 | −60.980 | 80.932 | 1.00 | 197.61 | O |
| ATOM | 1942 | CB | GLN | A | 283 | −43.779 | −59.146 | 79.388 | 1.00 | 198.32 | C |
| ATOM | 1943 | CG | GLN | A | 283 | −42.414 | −58.613 | 79.033 | 1.00 | 198.90 | C |
| ATOM | 1944 | CD | GLN | A | 283 | −42.081 | −57.333 | 79.755 | 1.00 | 199.31 | C |
| ATOM | 1945 | OE1 | GLN | A | 283 | −42.718 | −56.294 | 79.535 | 1.00 | 199.43 | O |
| ATOM | 1946 | NE2 | GLN | A | 283 | −41.066 | −57.392 | 80.622 | 1.00 | 199.57 | N |
| ATOM | 1947 | N | ALA | A | 284 | −43.717 | −60.862 | 81.982 | 1.00 | 197.24 | N |
| ATOM | 1948 | CA | ALA | A | 284 | −44.161 | −61.121 | 83.352 | 1.00 | 196.62 | C |
| ATOM | 1949 | C | ALA | A | 284 | −45.095 | −60.057 | 83.917 | 1.00 | 196.07 | C |
| ATOM | 1950 | O | ALA | A | 284 | −46.021 | −60.380 | 84.675 | 1.00 | 196.03 | O |
| ATOM | 1951 | CB | ALA | A | 284 | −42.948 | −61.277 | 84.266 | 1.00 | 196.68 | C |
| ATOM | 1952 | N | SER | A | 285 | −44.839 | −58.799 | 83.554 | 1.00 | 195.21 | N |
| ATOM | 1953 | CA | SER | A | 285 | −45.518 | −57.666 | 84.156 | 1.00 | 194.55 | C |
| ATOM | 1954 | C | SER | A | 285 | −45.124 | −56.377 | 83.481 | 1.00 | 194.51 | C |
| ATOM | 1955 | O | SER | A | 285 | −43.957 | −55.977 | 83.555 | 1.00 | 194.56 | O |
| ATOM | 1956 | CB | SER | A | 285 | −45.093 | −57.534 | 85.603 | 1.00 | 194.51 | C |
| ATOM | 1957 | OG | SER | A | 285 | −43.908 | −56.765 | 85.681 | 1.00 | 193.65 | O |
| ATOM | 1958 | N | LEU | A | 286 | −46.104 | −55.706 | 82.874 | 1.00 | 194.36 | N |
| ATOM | 1959 | CA | LEU | A | 286 | −45.901 | −54.433 | 82.156 | 1.00 | 194.07 | C |
| ATOM | 1960 | C | LEU | A | 286 | −45.106 | −53.431 | 82.982 | 1.00 | 193.92 | C |
| ATOM | 1961 | O | LEU | A | 286 | −45.426 | −53.147 | 84.134 | 1.00 | 193.89 | O |
| ATOM | 1962 | CB | LEU | A | 286 | −47.242 | −53.814 | 81.738 | 1.00 | 194.00 | C |
| ATOM | 1963 | CG | LEU | A | 286 | −48.400 | −54.624 | 81.113 | 1.00 | 194.06 | C |
| ATOM | 1964 | CD1 | LEU | A | 286 | −47.966 | −55.352 | 79.846 | 1.00 | 194.43 | C |
| ATOM | 1965 | CD2 | LEU | A | 286 | −49.135 | −55.570 | 82.100 | 1.00 | 192.66 | C |
| ATOM | 1966 | N | GLU | A | 287 | −44.062 | −52.896 | 82.386 | 1.00 | 193.80 | N |
| ATOM | 1967 | CA | GLU | A | 287 | −43.104 | −52.148 | 83.153 | 1.00 | 194.10 | C |
| ATOM | 1968 | C | GLU | A | 287 | −42.978 | −50.750 | 82.583 | 1.00 | 193.98 | C |
| ATOM | 1969 | O | GLU | A | 287 | −42.417 | −50.566 | 81.513 | 1.00 | 194.25 | O |
| ATOM | 1970 | CB | GLU | A | 287 | −41.780 | −52.890 | 83.109 | 1.00 | 194.25 | C |
| ATOM | 1971 | CG | GLU | A | 287 | −41.364 | −53.350 | 81.713 | 1.00 | 195.91 | C |
| ATOM | 1972 | CD | GLU | A | 287 | −39.866 | −53.628 | 81.603 | 1.00 | 198.47 | C |
| ATOM | 1973 | OE1 | GLU | A | 287 | −39.345 | −54.392 | 82.458 | 1.00 | 199.40 | O |
| ATOM | 1974 | OE2 | GLU | A | 287 | −39.217 | −53.090 | 80.660 | 1.00 | 198.92 | O |
| ATOM | 1975 | N | ILE | A | 288 | −43.515 | −49.756 | 83.279 | 1.00 | 193.92 | N |
| ATOM | 1976 | CA | ILE | A | 288 | −43.646 | −48.430 | 82.673 | 1.00 | 193.91 | C |
| ATOM | 1977 | C | ILE | A | 288 | −42.749 | −47.352 | 83.272 | 1.00 | 194.19 | C |
| ATOM | 1978 | O | ILE | A | 288 | −42.579 | −47.250 | 84.489 | 1.00 | 194.08 | O |
| ATOM | 1979 | CB | ILE | A | 288 | −45.102 | −47.944 | 82.647 | 1.00 | 193.74 | C |
| ATOM | 1980 | CG1 | ILE | A | 288 | −46.046 | −49.113 | 82.399 | 1.00 | 193.52 | C |
| ATOM | 1981 | CG2 | ILE | A | 288 | −45.287 | −46.921 | 81.550 | 1.00 | 193.74 | C |
| ATOM | 1982 | CD1 | ILE | A | 288 | −47.490 | −48.761 | 82.561 | 1.00 | 193.47 | C |
| ATOM | 1983 | N | SER | A | 289 | −42.200 | −46.536 | 82.376 | 1.00 | 194.62 | N |
| ATOM | 1984 | CA | SER | A | 289 | −41.236 | −45.485 | 82.708 | 1.00 | 195.00 | C |
| ATOM | 1985 | C | SER | A | 289 | −41.705 | −44.088 | 82.224 | 1.00 | 195.10 | C |
| ATOM | 1986 | O | SER | A | 289 | −42.782 | −43.987 | 81.630 | 1.00 | 194.96 | O |
| ATOM | 1987 | CB | SER | A | 289 | −39.850 | −45.857 | 82.141 | 1.00 | 195.14 | C |
| ATOM | 1988 | OG | SER | A | 289 | −39.890 | −46.219 | 80.766 | 1.00 | 195.19 | O |
| ATOM | 1989 | N | PRO | A | 290 | −40.902 | −43.014 | 82.476 | 1.00 | 195.25 | N |
| ATOM | 1990 | CA | PRO | A | 290 | −41.219 | −41.641 | 82.111 | 1.00 | 195.09 | C |
| ATOM | 1991 | C | PRO | A | 290 | −42.365 | −41.501 | 81.127 | 1.00 | 194.80 | C |
| ATOM | 1992 | O | PRO | A | 290 | −43.510 | −41.340 | 81.563 | 1.00 | 194.90 | O |
| ATOM | 1993 | CB | PRO | A | 290 | −39.900 | −41.164 | 81.500 | 1.00 | 195.17 | C |
| ATOM | 1994 | CG | PRO | A | 290 | −38.833 | −41.912 | 82.329 | 1.00 | 195.35 | C |
| ATOM | 1995 | CD | PRO | A | 290 | −39.565 | −43.026 | 83.100 | 1.00 | 195.44 | C |
| ATOM | 1996 | N | ILE | A | 291 | −42.056 | −41.557 | 79.828 | 1.00 | 194.26 | N |
| ATOM | 1997 | CA | ILE | A | 291 | −43.081 | −41.540 | 78.780 | 1.00 | 193.60 | C |
| ATOM | 1998 | C | ILE | A | 291 | −42.944 | −42.770 | 77.893 | 1.00 | 193.39 | C |
| ATOM | 1999 | O | ILE | A | 291 | −41.967 | −42.908 | 77.162 | 1.00 | 193.51 | O |
| ATOM | 2000 | CB | ILE | A | 291 | −43.078 | −40.228 | 77.944 | 1.00 | 193.41 | C |
| ATOM | 2001 | CG1 | ILE | A | 291 | −43.957 | −40.386 | 76.703 | 1.00 | 193.08 | C |
| ATOM | 2002 | CG2 | ILE | A | 291 | −41.665 | −39.790 | 77.578 | 1.00 | 192.89 | C |
| ATOM | 2003 | CD1 | ILE | A | 291 | −45.435 | −40.382 | 77.007 | 1.00 | 192.79 | C |
| ATOM | 2004 | N | THR | A | 292 | −43.928 | −43.661 | 77.975 | 1.00 | 192.95 | N |
| ATOM | 2005 | CA | THR | A | 292 | −43.813 | −44.982 | 77.370 | 1.00 | 192.66 | C |
| ATOM | 2006 | C | THR | A | 292 | −45.134 | −45.432 | 76.799 | 1.00 | 192.07 | C |
| ATOM | 2007 | O | THR | A | 292 | −46.168 | −45.330 | 77.456 | 1.00 | 191.71 | O |
| ATOM | 2008 | CB | THR | A | 292 | −43.378 | −46.070 | 78.408 | 1.00 | 192.94 | C |
| ATOM | 2009 | OG1 | THR | A | 292 | −42.537 | −45.491 | 79.424 | 1.00 | 193.32 | O |
| ATOM | 2010 | CG2 | THR | A | 292 | −42.653 | −47.260 | 77.717 | 1.00 | 193.27 | C |
| ATOM | 2011 | N | PHE | A | 293 | −45.093 | −45.931 | 75.569 | 1.00 | 191.64 | N |
| ATOM | 2012 | CA | PHE | A | 293 | −46.222 | −46.673 | 75.021 | 1.00 | 191.21 | C |
| ATOM | 2013 | C | PHE | A | 293 | −45.860 | −47.713 | 73.977 | 1.00 | 190.60 | C |
| ATOM | 2014 | O | PHE | A | 293 | −45.450 | −47.396 | 72.850 | 1.00 | 190.61 | O |
| ATOM | 2015 | CB | PHE | A | 293 | −47.391 | −45.782 | 74.555 | 1.00 | 191.46 | C |
| ATOM | 2016 | CG | PHE | A | 293 | −46.984 | −44.445 | 73.988 | 1.00 | 192.35 | C |
| ATOM | 2017 | CD1 | PHE | A | 293 | −46.066 | −43.617 | 74.649 | 1.00 | 193.40 | C |
| ATOM | 2018 | CD2 | PHE | A | 293 | −47.573 | −43.978 | 72.818 | 1.00 | 193.09 | C |
| ATOM | 2019 | CE1 | PHE | A | 293 | −45.706 | −42.365 | 74.128 | 1.00 | 193.57 | C |
| ATOM | 2020 | CE2 | PHE | A | 293 | −47.223 | −42.726 | 72.288 | 1.00 | 193.66 | C |
| ATOM | 2021 | CZ | PHE | A | 293 | −46.287 | −41.919 | 72.946 | 1.00 | 193.19 | C |
| ATOM | 2022 | N | LEU | A | 294 | −45.970 | −48.963 | 74.404 | 1.00 | 189.74 | N |
| ATOM | 2023 | CA | LEU | A | 294 | −46.305 | −50.029 | 73.485 | 1.00 | 189.04 | C |
| ATOM | 2024 | C | LEU | A | 294 | −47.501 | −50.755 | 74.102 | 1.00 | 188.25 | C |
| ATOM | 2025 | O | LEU | A | 294 | −47.893 | −50.484 | 75.242 | 1.00 | 187.39 | O |
| ATOM | 2026 | CB | LEU | A | 294 | −45.092 | −50.930 | 73.092 | 1.00 | 189.55 | C |
| ATOM | 2027 | CG | LEU | A | 294 | −44.992 | −51.828 | 71.814 | 1.00 | 189.39 | C |
| ATOM | 2028 | CD1 | LEU | A | 294 | −45.234 | −51.115 | 70.466 | 1.00 | 188.40 | C |
| ATOM | 2029 | CD2 | LEU | A | 294 | −43.645 | −52.554 | 71.802 | 1.00 | 188.48 | C |
| ATOM | 2030 | N | THR | A | 295 | −48.058 | −51.666 | 73.311 | 1.00 | 187.72 | N |
| ATOM | 2031 | CA | THR | A | 295 | −49.418 | −52.154 | 73.450 | 1.00 | 187.10 | C |
| ATOM | 2032 | C | THR | A | 295 | −49.460 | −53.678 | 73.603 | 1.00 | 186.56 | C |
| ATOM | 2033 | O | THR | A | 295 | −48.759 | −54.405 | 72.890 | 1.00 | 186.29 | O |
| ATOM | 2034 | CB | THR | A | 295 | −50.271 | −51.685 | 72.240 | 1.00 | 187.18 | C |
| ATOM | 2035 | OG1 | THR | A | 295 | −51.482 | −52.441 | 72.177 | 1.00 | 187.27 | O |
| ATOM | 2036 | CG2 | THR | A | 295 | −49.493 | −51.820 | 70.908 | 1.00 | 186.93 | C |
| ATOM | 2037 | N | ALA | A | 296 | −50.307 | −54.139 | 74.524 | 1.00 | 186.01 | N |
| ATOM | 2038 | CA | ALA | A | 296 | −50.226 | −55.497 | 75.070 | 1.00 | 185.48 | C |
| ATOM | 2039 | C | ALA | A | 296 | −51.575 | −56.157 | 75.340 | 1.00 | 185.05 | C |
| ATOM | 2040 | O | ALA | A | 296 | −52.596 | −55.477 | 75.495 | 1.00 | 184.89 | O |
| ATOM | 2041 | CB | ALA | A | 296 | −49.397 | −55.487 | 76.343 | 1.00 | 185.60 | C |
| ATOM | 2042 | N | GLN | A | 297 | −51.555 | −57.487 | 75.433 | 1.00 | 184.48 | N |
| ATOM | 2043 | CA | GLN | A | 297 | −52.777 | −58.270 | 75.525 | 1.00 | 184.20 | C |
| ATOM | 2044 | C | GLN | A | 297 | −52.668 | −59.404 | 76.517 | 1.00 | 183.87 | C |
| ATOM | 2045 | O | GLN | A | 297 | −51.553 | −59.829 | 76.826 | 1.00 | 183.99 | O |
| ATOM | 2046 | CB | GLN | A | 297 | −53.111 | −58.861 | 74.170 | 1.00 | 184.28 | C |
| ATOM | 2047 | CG | GLN | A | 297 | −52.210 | −59.985 | 73.754 | 1.00 | 184.98 | C |
| ATOM | 2048 | CD | GLN | A | 297 | −52.860 | −60.874 | 72.724 | 1.00 | 187.01 | C |
| ATOM | 2049 | OE1 | GLN | A | 297 | −53.805 | −61.615 | 73.031 | 1.00 | 187.37 | O |
| ATOM | 2050 | NE2 | GLN | A | 297 | −52.355 | −60.817 | 71.485 | 1.00 | 188.03 | N |
| ATOM | 2051 | N | THR | A | 298 | −53.834 | −59.889 | 76.977 | 1.00 | 183.47 | N |
| ATOM | 2052 | CA | THR | A | 298 | −53.977 | −61.006 | 77.939 | 1.00 | 182.95 | C |
| ATOM | 2053 | C | THR | A | 298 | −55.212 | −61.856 | 77.713 | 1.00 | 183.13 | C |
| ATOM | 2054 | O | THR | A | 298 | −56.173 | −61.425 | 77.073 | 1.00 | 183.19 | O |
| ATOM | 2055 | CB | THR | A | 298 | −54.235 | −60.507 | 79.328 | 1.00 | 182.63 | C |
| ATOM | 2056 | OG1 | THR | A | 298 | −55.174 | −59.434 | 79.249 | 1.00 | 182.08 | O |
| ATOM | 2057 | CG2 | THR | A | 298 | −52.972 | −60.072 | 79.981 | 1.00 | 182.19 | C |
| ATOM | 2058 | N | LEU | A | 299 | −55.198 | −63.042 | 78.311 | 1.00 | 183.18 | N |
| ATOM | 2059 | CA | LEU | A | 299 | −56.347 | −63.926 | 78.302 | 1.00 | 183.53 | C |
| ATOM | 2060 | C | LEU | A | 299 | −57.116 | −63.777 | 79.607 | 1.00 | 183.91 | C |
| ATOM | 2061 | O | LEU | A | 299 | −56.937 | −62.778 | 80.302 | 1.00 | 184.04 | O |
| ATOM | 2062 | CB | LEU | A | 299 | −55.884 | −65.362 | 78.115 | 1.00 | 183.46 | C |
| ATOM | 2063 | CG | LEU | A | 299 | −55.394 | −65.825 | 76.738 | 1.00 | 183.71 | C |
| ATOM | 2064 | CD1 | LEU | A | 299 | −56.544 | −65.884 | 75.722 | 1.00 | 183.84 | C |
| ATOM | 2065 | CD2 | LEU | A | 299 | −54.178 | −65.025 | 76.209 | 1.00 | 183.86 | C |
| ATOM | 2066 | N | LEU | A | 300 | −57.960 | −64.760 | 79.943 | 1.00 | 184.39 | N |
| ATOM | 2067 | CA | LEU | A | 300 | −58.804 | −64.701 | 81.146 | 1.00 | 184.72 | C |
| ATOM | 2068 | C | LEU | A | 300 | −59.210 | −66.072 | 81.725 | 1.00 | 185.41 | C |
| ATOM | 2069 | O | LEU | A | 300 | −59.093 | −67.105 | 81.058 | 1.00 | 185.20 | O |
| ATOM | 2070 | CB | LEU | A | 300 | −60.029 | −63.832 | 80.850 | 1.00 | 184.38 | C |
| ATOM | 2071 | CG | LEU | A | 300 | −59.680 | −62.414 | 80.366 | 1.00 | 183.57 | C |
| ATOM | 2072 | CD1 | LEU | A | 300 | −60.754 | −61.779 | 79.530 | 1.00 | 182.80 | C |
| ATOM | 2073 | CD2 | LEU | A | 300 | −59.308 | −61.517 | 81.524 | 1.00 | 183.16 | C |
| ATOM | 2074 | N | MET | A | 301 | −59.658 | −66.074 | 82.981 | 1.00 | 186.51 | N |
| ATOM | 2075 | CA | MET | A | 301 | −60.143 | −67.297 | 83.644 | 1.00 | 187.69 | C |
| ATOM | 2076 | C | MET | A | 301 | −61.643 | −67.236 | 84.024 | 1.00 | 188.10 | C |
| ATOM | 2077 | O | MET | A | 301 | −62.442 | −67.974 | 83.440 | 1.00 | 188.03 | O |
| ATOM | 2078 | CB | MET | A | 301 | −59.261 | −67.658 | 84.848 | 1.00 | 188.09 | C |
| ATOM | 2079 | CG | MET | A | 301 | −59.605 | −68.995 | 85.533 | 1.00 | 189.41 | C |
| ATOM | 2080 | SD | MET | A | 301 | −58.797 | −70.463 | 84.811 | 1.00 | 192.97 | S |
| ATOM | 2081 | CE | MET | A | 301 | −59.150 | −71.764 | 86.019 | 1.00 | 190.60 | C |
| ATOM | 2082 | N | ASP | A | 302 | −62.005 | −66.385 | 85.002 | 1.00 | 188.70 | N |
| ATOM | 2083 | CA | ASP | A | 302 | −63.423 | −66.027 | 85.322 | 1.00 | 189.36 | C |
| ATOM | 2084 | C | ASP | A | 302 | −63.691 | −64.815 | 86.256 | 1.00 | 189.56 | C |
| ATOM | 2085 | O | ASP | A | 302 | −62.739 | −64.126 | 86.632 | 1.00 | 189.65 | O |
| ATOM | 2086 | CB | ASP | A | 302 | −64.248 | −67.235 | 85.753 | 1.00 | 189.21 | C |
| ATOM | 2087 | CG | ASP | A | 302 | −65.412 | −67.470 | 84.826 | 1.00 | 189.48 | C |
| ATOM | 2088 | OD1 | ASP | A | 302 | −65.178 | −67.676 | 83.615 | 1.00 | 189.79 | O |
| ATOM | 2089 | OD2 | ASP | A | 302 | −66.566 | −67.406 | 85.294 | 1.00 | 189.56 | O |
| ATOM | 2090 | N | LEU | A | 303 | −64.980 | −64.586 | 86.612 | 1.00 | 189.97 | N |
| ATOM | 2091 | CA | LEU | A | 303 | −65.533 | −63.321 | 87.268 | 1.00 | 190.28 | C |
| ATOM | 2092 | C | LEU | A | 303 | −64.585 | −62.538 | 88.186 | 1.00 | 190.83 | C |
| ATOM | 2093 | O | LEU | A | 303 | −64.671 | −61.299 | 88.276 | 1.00 | 190.78 | O |
| ATOM | 2094 | CB | LEU | A | 303 | −66.900 | −63.543 | 88.009 | 1.00 | 190.24 | C |
| ATOM | 2095 | CG | LEU | A | 303 | −67.795 | −62.385 | 88.571 | 1.00 | 189.67 | C |
| ATOM | 2096 | CD1 | LEU | A | 303 | −69.229 | −62.844 | 88.819 | 1.00 | 188.43 | C |
| ATOM | 2097 | CD2 | LEU | A | 303 | −67.278 | −61.646 | 89.831 | 1.00 | 188.19 | C |
| ATOM | 2098 | N | GLY | A | 304 | −63.720 | −63.278 | 88.883 | 1.00 | 191.25 | N |
| ATOM | 2099 | CA | GLY | A | 304 | −62.716 | −62.706 | 89.763 | 1.00 | 191.49 | C |
| ATOM | 2100 | C | GLY | A | 304 | −62.254 | −61.396 | 89.183 | 1.00 | 191.52 | C |
| ATOM | 2101 | O | GLY | A | 304 | −61.472 | −61.359 | 88.227 | 1.00 | 191.15 | O |
| ATOM | 2102 | N | GLN | A | 305 | −62.794 | −60.312 | 89.720 | 1.00 | 191.82 | N |
| ATOM | 2103 | CA | GLN | A | 305 | −62.247 | −59.037 | 89.360 | 1.00 | 192.11 | C |
| ATOM | 2104 | C | GLN | A | 305 | −60.817 | −59.065 | 89.856 | 1.00 | 192.23 | C |
| ATOM | 2105 | O | GLN | A | 305 | −60.555 | −59.170 | 91.063 | 1.00 | 192.39 | O |
| ATOM | 2106 | CB | GLN | A | 305 | −63.070 | −57.841 | 89.868 | 1.00 | 192.29 | C |
| ATOM | 2107 | CG | GLN | A | 305 | −63.615 | −57.863 | 91.285 | 1.00 | 192.07 | C |
| ATOM | 2108 | CD | GLN | A | 305 | −64.418 | −56.602 | 91.556 | 1.00 | 191.95 | C |
| ATOM | 2109 | OE1 | GLN | A | 305 | −65.430 | −56.344 | 90.900 | 1.00 | 191.57 | O |
| ATOM | 2110 | NE2 | GLN | A | 305 | −63.957 | −55.798 | 92.502 | 1.00 | 191.85 | N |
| ATOM | 2111 | N | PHE | A | 306 | −59.896 | −59.056 | 88.901 | 1.00 | 192.28 | N |
| ATOM | 2112 | CA | PHE | A | 306 | −58.503 | −59.202 | 89.249 | 1.00 | 192.48 | C |
| ATOM | 2113 | C | PHE | A | 306 | −57.821 | −57.887 | 89.654 | 1.00 | 192.65 | C |
| ATOM | 2114 | O | PHE | A | 306 | −58.115 | −56.775 | 89.167 | 1.00 | 192.34 | O |
| ATOM | 2115 | CB | PHE | A | 306 | −57.713 | −60.029 | 88.215 | 1.00 | 192.52 | C |
| ATOM | 2116 | CG | PHE | A | 306 | −58.333 | −61.385 | 87.900 | 1.00 | 192.49 | C |
| ATOM | 2117 | CD1 | PHE | A | 306 | −58.464 | −61.816 | 86.576 | 1.00 | 192.76 | C |
| ATOM | 2118 | CD2 | PHE | A | 306 | −58.778 | −62.226 | 88.913 | 1.00 | 192.62 | C |
| ATOM | 2119 | CE1 | PHE | A | 306 | −59.031 | −63.051 | 86.263 | 1.00 | 192.39 | C |
| ATOM | 2120 | CE2 | PHE | A | 306 | −59.347 | −63.467 | 88.612 | 1.00 | 192.76 | C |
| ATOM | 2121 | CZ | PHE | A | 306 | −59.476 | −63.873 | 87.282 | 1.00 | 192.66 | C |
| ATOM | 2122 | N | LEU | A | 307 | −56.912 | −58.086 | 90.594 | 1.00 | 192.99 | N |
| ATOM | 2123 | CA | LEU | A | 307 | −56.305 | −57.080 | 91.438 | 1.00 | 193.10 | C |
| ATOM | 2124 | C | LEU | A | 307 | −55.271 | −56.331 | 90.570 | 1.00 | 193.22 | C |
| ATOM | 2125 | O | LEU | A | 307 | −54.567 | −56.988 | 89.793 | 1.00 | 193.37 | O |
| ATOM | 2126 | CB | LEU | A | 307 | −55.677 | −57.848 | 92.646 | 1.00 | 193.11 | C |
| ATOM | 2127 | CG | LEU | A | 307 | −56.130 | −59.314 | 93.017 | 1.00 | 191.94 | C |
| ATOM | 2128 | CD1 | LEU | A | 307 | −55.023 | −60.183 | 93.678 | 1.00 | 190.15 | C |
| ATOM | 2129 | CD2 | LEU | A | 307 | −57.447 | −59.416 | 93.829 | 1.00 | 190.43 | C |
| ATOM | 2130 | N | LEU | A | 308 | −55.184 | −54.993 | 90.650 | 1.00 | 193.34 | N |
| ATOM | 2131 | CA | LEU | A | 308 | −54.300 | −54.221 | 89.698 | 1.00 | 193.43 | C |
| ATOM | 2132 | C | LEU | A | 308 | −53.196 | −53.306 | 90.276 | 1.00 | 193.90 | C |
| ATOM | 2133 | O | LEU | A | 308 | −53.411 | −52.101 | 90.476 | 1.00 | 193.87 | O |
| ATOM | 2134 | CB | LEU | A | 308 | −55.128 | −53.409 | 88.691 | 1.00 | 193.04 | C |
| ATOM | 2135 | CG | LEU | A | 308 | −54.593 | −53.185 | 87.275 | 1.00 | 191.29 | C |
| ATOM | 2136 | CD1 | LEU | A | 308 | −53.224 | −53.771 | 87.054 | 1.00 | 189.49 | C |
| ATOM | 2137 | CD2 | LEU | A | 308 | −55.547 | −53.790 | 86.308 | 1.00 | 189.92 | C |
| ATOM | 2138 | N | PHE | A | 309 | −52.001 | −53.867 | 90.447 | 1.00 | 194.38 | N |
| ATOM | 2139 | CA | PHE | A | 309 | −50.942 | −53.232 | 91.229 | 1.00 | 195.02 | C |
| ATOM | 2140 | C | PHE | A | 309 | −50.157 | −52.151 | 90.513 | 1.00 | 194.97 | C |
| ATOM | 2141 | O | PHE | A | 309 | −50.170 | −52.067 | 89.295 | 1.00 | 194.96 | O |
| ATOM | 2142 | CB | PHE | A | 309 | −49.914 | −54.269 | 91.670 | 1.00 | 195.56 | C |
| ATOM | 2143 | CG | PHE | A | 309 | −50.483 | −55.437 | 92.404 | 1.00 | 196.50 | C |
| ATOM | 2144 | CD1 | PHE | A | 309 | −50.671 | −56.656 | 91.747 | 1.00 | 197.38 | C |
| ATOM | 2145 | CD2 | PHE | A | 309 | −50.785 | −55.343 | 93.764 | 1.00 | 198.21 | C |
| ATOM | 2146 | CE1 | PHE | A | 309 | −51.180 | −57.776 | 92.423 | 1.00 | 198.69 | C |
| ATOM | 2147 | CE2 | PHE | A | 309 | −51.300 | −56.458 | 94.470 | 1.00 | 199.67 | C |
| ATOM | 2148 | CZ | PHE | A | 309 | −51.499 | −57.683 | 93.796 | 1.00 | 198.92 | C |
| ATOM | 2149 | N | CYS | A | 310 | −49.472 | −51.337 | 91.312 | 1.00 | 195.09 | N |
| ATOM | 2150 | CA | CYS | A | 310 | −48.287 | −50.614 | 90.892 | 1.00 | 195.05 | C |
| ATOM | 2151 | C | CYS | A | 310 | −47.108 | −51.334 | 91.540 | 1.00 | 195.00 | C |
| ATOM | 2152 | O | CYS | A | 310 | −45.962 | −51.175 | 91.138 | 1.00 | 194.79 | O |
| ATOM | 2153 | CB | CYS | A | 310 | −48.346 | −49.165 | 91.361 | 1.00 | 195.16 | C |
| ATOM | 2154 | SG | CYS | A | 310 | −47.034 | −48.115 | 90.677 | 1.00 | 195.54 | S |
| ATOM | 2155 | N | HIS | A | 311 | −47.424 | −52.118 | 92.564 | 1.00 | 195.20 | N |
| ATOM | 2156 | CA | HIS | A | 311 | −46.495 | −53.023 | 93.238 | 1.00 | 195.59 | C |
| ATOM | 2157 | C | HIS | A | 311 | −45.058 | −52.519 | 93.400 | 1.00 | 195.54 | C |
| ATOM | 2158 | O | HIS | A | 311 | −44.112 | −53.156 | 92.951 | 1.00 | 195.64 | O |
| ATOM | 2159 | CB | HIS | A | 311 | −46.502 | −54.388 | 92.549 | 1.00 | 195.80 | C |
| ATOM | 2160 | CG | HIS | A | 311 | −45.972 | −55.504 | 93.398 | 1.00 | 197.14 | C |
| ATOM | 2161 | ND1 | HIS | A | 311 | −44.623 | −55.709 | 93.607 | 1.00 | 198.44 | N |
| ATOM | 2162 | CD2 | HIS | A | 311 | −46.609 | −56.490 | 94.075 | 1.00 | 198.41 | C |
| ATOM | 2163 | CE1 | HIS | A | 311 | −44.453 | −56.768 | 94.380 | 1.00 | 199.05 | C |
| ATOM | 2164 | NE2 | HIS | A | 311 | −45.642 | −57.261 | 94.679 | 1.00 | 199.18 | N |
| ATOM | 2165 | N | ILE | A | 312 | −44.903 | −51.363 | 94.032 | 1.00 | 195.60 | N |
| ATOM | 2166 | CA | ILE | A | 312 | −43.599 | −50.924 | 94.540 | 1.00 | 195.44 | C |
| ATOM | 2167 | C | ILE | A | 312 | −43.824 | −50.285 | 95.918 | 1.00 | 195.69 | C |
| ATOM | 2168 | O | ILE | A | 312 | −44.887 | −49.714 | 96.174 | 1.00 | 195.75 | O |
| ATOM | 2169 | CB | ILE | A | 312 | −42.864 | −49.943 | 93.577 | 1.00 | 195.24 | C |
| ATOM | 2170 | CG1 | ILE | A | 312 | −43.395 | −50.065 | 92.144 | 1.00 | 194.23 | C |
| ATOM | 2171 | CG2 | ILE | A | 312 | −41.348 | −50.147 | 93.666 | 1.00 | 195.03 | C |
| ATOM | 2172 | CD1 | ILE | A | 312 | −42.432 | −49.673 | 91.080 | 1.00 | 193.39 | C |
| ATOM | 2173 | N | SER | A | 313 | −42.845 | −50.391 | 96.811 | 1.00 | 195.77 | N |
| ATOM | 2174 | CA | SER | A | 313 | −43.023 | −49.877 | 98.167 | 1.00 | 195.98 | C |
| ATOM | 2175 | C | SER | A | 313 | −43.455 | −48.414 | 98.209 | 1.00 | 196.13 | C |
| ATOM | 2176 | O | SER | A | 313 | −44.453 | −48.082 | 98.840 | 1.00 | 196.16 | O |
| ATOM | 2177 | CB | SER | A | 313 | −41.767 | −50.099 | 99.010 | 1.00 | 195.95 | C |
| ATOM | 2178 | OG | SER | A | 313 | −41.738 | −51.423 | 99.519 | 1.00 | 196.00 | O |
| ATOM | 2179 | N | SER | A | 314 | −42.717 | −47.556 | 97.511 | 1.00 | 196.46 | N |
| ATOM | 2180 | CA | SER | A | 314 | −42.922 | −46.102 | 97.556 | 1.00 | 196.85 | C |
| ATOM | 2181 | C | SER | A | 314 | −44.234 | −45.621 | 96.924 | 1.00 | 197.01 | C |
| ATOM | 2182 | O | SER | A | 314 | −44.953 | −44.792 | 97.499 | 1.00 | 197.03 | O |
| ATOM | 2183 | CB | SER | A | 314 | −41.740 | −45.389 | 96.885 | 1.00 | 196.94 | C |
| ATOM | 2184 | OG | SER | A | 314 | −41.647 | −45.725 | 95.506 | 1.00 | 197.02 | O |
| ATOM | 2185 | N | HIS | A | 315 | −44.525 | −46.135 | 95.732 | 1.00 | 197.21 | N |
| ATOM | 2186 | CA | HIS | A | 315 | −45.694 | −45.723 | 94.959 | 1.00 | 197.39 | C |
| ATOM | 2187 | C | HIS | A | 315 | −46.972 | −46.280 | 95.578 | 1.00 | 197.60 | C |
| ATOM | 2188 | O | HIS | A | 315 | −48.047 | −45.702 | 95.429 | 1.00 | 197.42 | O |
| ATOM | 2189 | CB | HIS | A | 315 | −45.557 | −46.198 | 93.508 | 1.00 | 197.31 | C |
| ATOM | 2190 | CG | HIS | A | 315 | −44.247 | −45.841 | 92.871 | 1.00 | 196.79 | C |
| ATOM | 2191 | ND1 | HIS | A | 315 | −43.123 | −46.630 | 92.989 | 1.00 | 196.23 | N |
| ATOM | 2192 | CD2 | HIS | A | 315 | −43.886 | −44.785 | 92.103 | 1.00 | 196.44 | C |
| ATOM | 2193 | CE1 | HIS | A | 315 | −42.123 | −46.070 | 92.331 | 1.00 | 196.29 | C |
| ATOM | 2194 | NE2 | HIS | A | 315 | −42.560 | −44.951 | 91.781 | 1.00 | 196.35 | N |
| ATOM | 2195 | N | GLN | A | 316 | −46.813 | −47.400 | 96.283 | 1.00 | 198.01 | N |
| ATOM | 2196 | CA | GLN | A | 316 | −47.896 | −48.145 | 96.924 | 1.00 | 198.41 | C |
| ATOM | 2197 | C | GLN | A | 316 | −48.914 | −47.257 | 97.653 | 1.00 | 198.71 | C |
| ATOM | 2198 | O | GLN | A | 316 | −49.894 | −46.845 | 97.043 | 1.00 | 198.81 | O |
| ATOM | 2199 | CB | GLN | A | 316 | −47.320 | −49.203 | 97.875 | 1.00 | 198.32 | C |
| ATOM | 2200 | CG | GLN | A | 316 | −48.193 | −50.423 | 98.055 | 1.00 | 198.15 | C |
| ATOM | 2201 | CD | GLN | A | 316 | −48.139 | −50.960 | 99.461 | 1.00 | 198.00 | C |
| ATOM | 2202 | OE1 | GLN | A | 316 | −47.591 | −52.031 | 99.700 | 1.00 | 198.28 | O |
| ATOM | 2203 | NE2 | GLN | A | 316 | −48.700 | −50.211 | 100.407 | 1.00 | 197.95 | N |
| ATOM | 2204 | N | HIS | A | 317 | −48.685 | −46.966 | 98.937 | 1.00 | 198.99 | N |
| ATOM | 2205 | CA | HIS | A | 317 | −49.615 | −46.162 | 99.749 | 1.00 | 199.28 | C |
| ATOM | 2206 | C | HIS | A | 317 | −50.478 | −45.219 | 98.882 | 1.00 | 199.07 | C |
| ATOM | 2207 | O | HIS | A | 317 | −51.708 | −45.329 | 98.895 | 1.00 | 199.27 | O |
| ATOM | 2208 | CB | HIS | A | 317 | −48.853 | −45.402 | 100.854 | 1.00 | 199.60 | C |
| ATOM | 2209 | CG | HIS | A | 317 | −49.732 | −44.766 | 101.900 | 1.00 | 200.91 | C |
| ATOM | 2210 | ND1 | HIS | A | 317 | −50.186 | −45.447 | 103.012 | 1.00 | 201.66 | N |
| ATOM | 2211 | CD2 | HIS | A | 317 | −50.202 | −43.497 | 102.025 | 1.00 | 201.93 | C |
| ATOM | 2212 | CE1 | HIS | A | 317 | −50.910 | −44.634 | 103.765 | 1.00 | 201.75 | C |
| ATOM | 2213 | NE2 | HIS | A | 317 | −50.936 | −43.444 | 103.189 | 1.00 | 201.92 | N |
| ATOM | 2214 | N | ASP | A | 318 | −49.839 | −44.334 | 98.106 | 1.00 | 198.62 | N |
| ATOM | 2215 | CA | ASP | A | 318 | −50.563 | −43.395 | 97.228 | 1.00 | 198.03 | C |
| ATOM | 2216 | C | ASP | A | 318 | −50.680 | −43.921 | 95.791 | 1.00 | 197.55 | C |
| ATOM | 2217 | O | ASP | A | 318 | −50.476 | −43.184 | 94.827 | 1.00 | 197.49 | O |
| ATOM | 2218 | CB | ASP | A | 318 | −49.911 | −41.996 | 97.265 | 1.00 | 198.09 | C |
| ATOM | 2219 | CG | ASP | A | 318 | −48.493 | −41.974 | 96.680 | 1.00 | 197.77 | C |
| ATOM | 2220 | OD1 | ASP | A | 318 | −48.086 | −40.893 | 96.199 | 1.00 | 197.13 | O |
| ATOM | 2221 | OD2 | ASP | A | 318 | −47.792 | −43.017 | 96.697 | 1.00 | 196.94 | O |
| ATOM | 2222 | N | GLY | A | 319 | −51.048 | −45.193 | 95.663 | 1.00 | 197.05 | N |
| ATOM | 2223 | CA | GLY | A | 319 | −50.882 | −45.939 | 94.409 | 1.00 | 196.41 | C |
| ATOM | 2224 | C | GLY | A | 319 | −52.088 | −46.212 | 93.526 | 1.00 | 195.80 | C |
| ATOM | 2225 | O | GLY | A | 319 | −53.189 | −45.695 | 93.749 | 1.00 | 195.92 | O |
| ATOM | 2226 | N | MET | A | 320 | −51.857 | −47.051 | 92.522 | 1.00 | 194.91 | N |
| ATOM | 2227 | CA | MET | A | 320 | −52.761 | −47.220 | 91.401 | 1.00 | 193.89 | C |
| ATOM | 2228 | C | MET | A | 320 | −53.477 | −48.560 | 91.509 | 1.00 | 193.24 | C |
| ATOM | 2229 | O | MET | A | 320 | −52.895 | −49.546 | 91.949 | 1.00 | 193.11 | O |
| ATOM | 2230 | CB | MET | A | 320 | −51.949 | −47.116 | 90.106 | 1.00 | 194.01 | C |
| ATOM | 2231 | CG | MET | A | 320 | −50.981 | −45.909 | 90.080 | 1.00 | 194.05 | C |
| ATOM | 2232 | SD | MET | A | 320 | −49.452 | −46.102 | 89.131 | 1.00 | 193.92 | S |
| ATOM | 2233 | CE | MET | A | 320 | −49.986 | −45.625 | 87.486 | 1.00 | 193.43 | C |
| ATOM | 2234 | N | GLU | A | 321 | −54.744 | −48.584 | 91.114 | 1.00 | 192.47 | N |
| ATOM | 2235 | CA | GLU | A | 321 | −55.592 | −49.756 | 91.286 | 1.00 | 191.97 | C |
| ATOM | 2236 | C | GLU | A | 321 | −56.734 | −49.752 | 90.277 | 1.00 | 191.27 | C |
| ATOM | 2237 | O | GLU | A | 321 | −57.157 | −48.690 | 89.841 | 1.00 | 191.06 | O |
| ATOM | 2238 | CB | GLU | A | 321 | −56.213 | −49.735 | 92.687 | 1.00 | 192.08 | C |
| ATOM | 2239 | CG | GLU | A | 321 | −55.360 | −50.274 | 93.859 | 1.00 | 192.51 | C |
| ATOM | 2240 | CD | GLU | A | 321 | −56.232 | −50.581 | 95.116 | 1.00 | 192.89 | C |
| ATOM | 2241 | OE1 | GLU | A | 321 | −57.477 | −50.383 | 95.068 | 1.00 | 193.68 | O |
| ATOM | 2242 | OE2 | GLU | A | 321 | −55.674 | −51.033 | 96.153 | 1.00 | 194.14 | O |
| ATOM | 2243 | N | ALA | A | 322 | −57.244 | −50.933 | 89.932 | 1.00 | 190.79 | N |
| ATOM | 2244 | CA | ALA | A | 322 | −58.519 | −51.066 | 89.209 | 1.00 | 190.57 | C |
| ATOM | 2245 | C | ALA | A | 322 | −58.944 | −52.516 | 89.007 | 1.00 | 190.62 | C |
| ATOM | 2246 | O | ALA | A | 322 | −58.122 | −53.432 | 89.032 | 1.00 | 190.58 | O |
| ATOM | 2247 | CB | ALA | A | 322 | −58.449 | −50.378 | 87.894 | 1.00 | 190.67 | C |
| ATOM | 2248 | N | TYR | A | 323 | −60.231 | −52.733 | 88.786 | 1.00 | 190.74 | N |
| ATOM | 2249 | CA | TYR | A | 323 | −60.716 | −54.107 | 88.727 | 1.00 | 191.22 | C |
| ATOM | 2250 | C | TYR | A | 323 | −60.921 | −54.650 | 87.310 | 1.00 | 191.37 | C |
| ATOM | 2251 | O | TYR | A | 323 | −60.533 | −54.001 | 86.349 | 1.00 | 191.42 | O |
| ATOM | 2252 | CB | TYR | A | 323 | −61.939 | −54.286 | 89.643 | 1.00 | 191.44 | C |
| ATOM | 2253 | CG | TYR | A | 323 | −61.543 | −54.364 | 91.112 | 1.00 | 191.55 | C |
| ATOM | 2254 | CD1 | TYR | A | 323 | −61.861 | −53.341 | 92.006 | 1.00 | 191.43 | C |
| ATOM | 2255 | CD2 | TYR | A | 323 | −60.812 | −55.450 | 91.594 | 1.00 | 191.63 | C |
| ATOM | 2256 | CE1 | TYR | A | 323 | −61.475 | −53.411 | 93.342 | 1.00 | 191.17 | C |
| ATOM | 2257 | CE2 | TYR | A | 323 | −60.427 | −55.528 | 92.920 | 1.00 | 191.56 | C |
| ATOM | 2258 | CZ | TYR | A | 323 | −60.756 | −54.509 | 93.785 | 1.00 | 191.40 | C |
| ATOM | 2259 | OH | TYR | A | 323 | −60.354 | −54.607 | 95.094 | 1.00 | 191.75 | O |
| ATOM | 2260 | N | VAL | A | 324 | −61.488 | −55.853 | 87.194 | 1.00 | 191.66 | N |
| ATOM | 2261 | CA | VAL | A | 324 | −61.688 | −56.541 | 85.907 | 1.00 | 191.96 | C |
| ATOM | 2262 | C | VAL | A | 324 | −62.677 | −57.718 | 85.989 | 1.00 | 192.77 | C |
| ATOM | 2263 | O | VAL | A | 324 | −62.268 | −58.858 | 86.193 | 1.00 | 192.72 | O |
| ATOM | 2264 | CB | VAL | A | 324 | −60.352 | −57.048 | 85.326 | 1.00 | 191.62 | C |
| ATOM | 2265 | CG1 | VAL | A | 324 | −60.061 | −56.381 | 84.022 | 1.00 | 191.12 | C |
| ATOM | 2266 | CG2 | VAL | A | 324 | −59.211 | −56.830 | 86.305 | 1.00 | 191.17 | C |
| ATOM | 2267 | N | LYS | A | 325 | −63.968 | −57.444 | 85.783 | 1.00 | 193.85 | N |
| ATOM | 2268 | CA | LYS | A | 325 | −65.068 | −58.402 | 86.095 | 1.00 | 195.17 | C |
| ATOM | 2269 | C | LYS | A | 325 | −65.246 | −59.677 | 85.207 | 1.00 | 195.44 | C |
| ATOM | 2270 | O | LYS | A | 325 | −66.273 | −60.359 | 85.313 | 1.00 | 195.65 | O |
| ATOM | 2271 | CB | LYS | A | 325 | −66.433 | −57.668 | 86.212 | 1.00 | 195.16 | C |
| ATOM | 2272 | CG | LYS | A | 325 | −66.453 | −56.327 | 86.970 | 1.00 | 195.40 | C |
| ATOM | 2273 | CD | LYS | A | 325 | −67.896 | −55.817 | 87.148 | 1.00 | 195.29 | C |
| ATOM | 2274 | CE | LYS | A | 325 | −68.041 | −54.322 | 86.790 | 1.00 | 195.32 | C |
| ATOM | 2275 | NZ | LYS | A | 325 | −67.357 | −53.364 | 87.718 | 1.00 | 194.77 | N |
| ATOM | 2276 | N | VAL | A | 326 | −64.270 | −59.985 | 84.350 | 1.00 | 195.84 | N |
| ATOM | 2277 | CA | VAL | A | 326 | −64.247 | −61.199 | 83.501 | 1.00 | 196.24 | C |
| ATOM | 2278 | C | VAL | A | 326 | −65.551 | −62.028 | 83.437 | 1.00 | 197.19 | C |
| ATOM | 2279 | O | VAL | A | 326 | −65.604 | −63.121 | 83.993 | 1.00 | 197.26 | O |
| ATOM | 2280 | CB | VAL | A | 326 | −63.108 | −62.142 | 83.971 | 1.00 | 196.17 | C |
| ATOM | 2281 | CG1 | VAL | A | 326 | −62.628 | −63.008 | 82.849 | 1.00 | 196.24 | C |
| ATOM | 2282 | CG2 | VAL | A | 326 | −61.948 | −61.360 | 84.519 | 1.00 | 195.94 | C |
| ATOM | 2283 | N | ASP | A | 327 | −66.591 | −61.547 | 82.759 | 1.00 | 198.32 | N |
| ATOM | 2284 | CA | ASP | A | 327 | −67.893 | −62.244 | 82.834 | 1.00 | 199.70 | C |
| ATOM | 2285 | C | ASP | A | 327 | −68.253 | −63.194 | 81.675 | 1.00 | 200.28 | C |
| ATOM | 2286 | O | ASP | A | 327 | −67.532 | −63.272 | 80.691 | 1.00 | 200.39 | O |
| ATOM | 2287 | CB | ASP | A | 327 | −69.038 | −61.258 | 83.093 | 1.00 | 199.93 | C |
| ATOM | 2288 | CG | ASP | A | 327 | −70.184 | −61.897 | 83.885 | 1.00 | 201.58 | C |
| ATOM | 2289 | OD1 | ASP | A | 327 | −70.070 | −62.021 | 85.129 | 1.00 | 203.41 | O |
| ATOM | 2290 | OD2 | ASP | A | 327 | −71.198 | −62.294 | 83.267 | 1.00 | 203.28 | O |
| ATOM | 2291 | N | SER | A | 328 | −69.366 | −63.924 | 81.830 | 1.00 | 201.29 | N |
| ATOM | 2292 | CA | SER | A | 328 | −69.950 | −64.806 | 80.802 | 1.00 | 202.24 | C |
| ATOM | 2293 | C | SER | A | 328 | −70.329 | −63.967 | 79.585 | 1.00 | 202.90 | C |
| ATOM | 2294 | O | SER | A | 328 | −70.229 | −62.739 | 79.634 | 1.00 | 202.85 | O |
| ATOM | 2295 | CB | SER | A | 328 | −71.206 | −65.521 | 81.337 | 1.00 | 202.40 | C |
| ATOM | 2296 | OG | SER | A | 328 | −71.189 | −65.720 | 82.747 | 1.00 | 202.57 | O |
| ATOM | 2297 | N | CYS | A | 329 | −70.801 | −64.600 | 78.509 | 1.00 | 203.91 | N |
| ATOM | 2298 | CA | CYS | A | 329 | −70.847 | −63.880 | 77.226 | 1.00 | 205.02 | C |
| ATOM | 2299 | C | CYS | A | 329 | −72.154 | −63.498 | 76.509 | 1.00 | 205.76 | C |
| ATOM | 2300 | O | CYS | A | 329 | −73.132 | −64.256 | 76.531 | 1.00 | 205.72 | O |
| ATOM | 2301 | CB | CYS | A | 329 | −69.873 | −64.497 | 76.233 | 1.00 | 204.85 | C |
| ATOM | 2302 | SG | CYS | A | 329 | −68.166 | −64.026 | 76.613 | 1.00 | 205.82 | S |
| ATOM | 2303 | N | PRO | A | 330 | −72.148 | −62.303 | 75.872 | 1.00 | 206.63 | N |
| ATOM | 2304 | CA | PRO | A | 330 | −73.126 | −61.848 | 74.879 | 1.00 | 207.35 | C |
| ATOM | 2305 | C | PRO | A | 330 | −73.191 | −62.689 | 73.585 | 1.00 | 208.07 | C |
| ATOM | 2306 | O | PRO | A | 330 | −74.264 | −63.204 | 73.265 | 1.00 | 208.17 | O |
| ATOM | 2307 | CB | PRO | A | 330 | −72.667 | −60.413 | 74.563 | 1.00 | 207.32 | C |
| ATOM | 2308 | CG | PRO | A | 330 | −71.866 | −59.997 | 75.751 | 1.00 | 206.97 | C |
| ATOM | 2309 | CD | PRO | A | 330 | −71.163 | −61.242 | 76.167 | 1.00 | 206.61 | C |
| ATOM | 2310 | N | GLU | A | 331 | −72.064 | −62.835 | 72.871 | 1.00 | 208.85 | N |
| ATOM | 2311 | CA | GLU | A | 331 | −72.027 | −63.396 | 71.489 | 1.00 | 209.54 | C |
| ATOM | 2312 | C | GLU | A | 331 | −72.593 | −64.826 | 71.265 | 1.00 | 209.98 | C |
| ATOM | 2313 | O | GLU | A | 331 | −72.608 | −65.334 | 70.132 | 1.00 | 210.02 | O |
| ATOM | 2314 | CB | GLU | A | 331 | −70.624 | −63.218 | 70.864 | 1.00 | 209.51 | C |
| ATOM | 2315 | CG | GLU | A | 331 | −70.549 | −63.317 | 69.317 | 1.00 | 209.70 | C |
| ATOM | 2316 | CD | GLU | A | 331 | −71.527 | −62.398 | 68.565 | 1.00 | 209.70 | C |
| ATOM | 2317 | OE1 | GLU | A | 331 | −71.750 | −62.631 | 67.354 | 1.00 | 209.63 | O |
| ATOM | 2318 | OE2 | GLU | A | 331 | −72.071 | −61.447 | 69.170 | 1.00 | 209.46 | O |
| ATOM | 2319 | N | GLU | A | 332 | −73.055 | −65.466 | 72.339 | 1.00 | 210.51 | N |
| ATOM | 2320 | CA | GLU | A | 332 | −73.971 | −66.607 | 72.225 | 1.00 | 210.96 | C |
| ATOM | 2321 | C | GLU | A | 332 | −75.374 | −66.208 | 72.746 | 1.00 | 211.32 | C |
| ATOM | 2322 | O | GLU | A | 332 | −75.550 | −66.029 | 73.964 | 1.00 | 211.46 | O |
| ATOM | 2323 | CB | GLU | A | 332 | −73.438 | −67.843 | 72.967 | 1.00 | 210.91 | C |
| ATOM | 2324 | CG | GLU | A | 332 | −72.418 | −68.671 | 72.192 | 1.00 | 210.70 | C |
| ATOM | 2325 | CD | GLU | A | 332 | −70.983 | −68.338 | 72.556 | 1.00 | 210.59 | C |
| ATOM | 2326 | OE1 | GLU | A | 332 | −70.265 | −69.259 | 73.005 | 1.00 | 210.54 | O |
| ATOM | 2327 | OE2 | GLU | A | 332 | −70.574 | −67.165 | 72.402 | 1.00 | 210.38 | O |
| ATOM | 2328 | N | PRO | A | 333 | −76.366 | −66.039 | 71.825 | 1.00 | 211.50 | N |
| ATOM | 2329 | CA | PRO | A | 333 | −77.746 | −65.692 | 72.213 | 1.00 | 211.46 | C |
| ATOM | 2330 | C | PRO | A | 333 | −78.610 | −66.922 | 72.509 | 1.00 | 211.43 | C |
| ATOM | 2331 | O | PRO | A | 333 | −78.661 | −67.387 | 73.650 | 1.00 | 211.39 | O |
| ATOM | 2332 | CB | PRO | A | 333 | −78.280 | −64.960 | 70.977 | 1.00 | 211.39 | C |
| ATOM | 2333 | CG | PRO | A | 333 | −77.540 | −65.580 | 69.819 | 1.00 | 211.46 | C |
| ATOM | 2334 | CD | PRO | A | 333 | −76.233 | −66.155 | 70.354 | 1.00 | 211.53 | C |
| ATOM | 2335 | N | LYS | A | 377 | −40.696 | −90.255 | 76.524 | 1.00 | 194.03 | N |
| ATOM | 2336 | CA | LYS | A | 377 | −40.070 | −88.929 | 76.581 | 1.00 | 194.11 | C |
| ATOM | 2337 | C | LYS | A | 377 | −41.125 | −87.852 | 76.918 | 1.00 | 193.94 | C |
| ATOM | 2338 | O | LYS | A | 377 | −41.138 | −86.749 | 76.350 | 1.00 | 193.72 | O |
| ATOM | 2339 | CB | LYS | A | 377 | −39.313 | −88.626 | 75.267 | 1.00 | 194.26 | C |
| ATOM | 2340 | CG | LYS | A | 377 | −38.637 | −89.851 | 74.566 | 1.00 | 194.32 | C |
| ATOM | 2341 | CD | LYS | A | 377 | −37.566 | −90.563 | 75.417 | 1.00 | 194.19 | C |
| ATOM | 2342 | CE | LYS | A | 377 | −36.230 | −89.823 | 75.398 | 1.00 | 194.12 | C |
| ATOM | 2343 | NZ | LYS | A | 377 | −35.330 | −90.220 | 76.523 | 1.00 | 193.98 | N |
| ATOM | 2344 | N | HIS | A | 378 | −41.978 | −88.199 | 77.886 | 1.00 | 193.88 | N |
| ATOM | 2345 | CA | HIS | A | 378 | −43.228 | −87.491 | 78.200 | 1.00 | 193.78 | C |
| ATOM | 2346 | C | HIS | A | 378 | −43.074 | −86.222 | 79.059 | 1.00 | 193.89 | C |
| ATOM | 2347 | O | HIS | A | 378 | −42.047 | −85.545 | 78.958 | 1.00 | 194.00 | O |
| ATOM | 2348 | CB | HIS | A | 378 | −44.238 | −88.472 | 78.815 | 1.00 | 193.55 | C |
| ATOM | 2349 | CG | HIS | A | 378 | −45.107 | −89.145 | 77.802 | 1.00 | 193.15 | C |
| ATOM | 2350 | ND1 | HIS | A | 378 | −45.576 | −88.495 | 76.679 | 1.00 | 192.87 | N |
| ATOM | 2351 | CD2 | HIS | A | 378 | −45.601 | −90.402 | 77.743 | 1.00 | 192.88 | C |
| ATOM | 2352 | CE1 | HIS | A | 378 | −46.310 | −89.327 | 75.965 | 1.00 | 192.77 | C |
| ATOM | 2353 | NE2 | HIS | A | 378 | −46.344 | −90.490 | 76.590 | 1.00 | 193.05 | N |
| ATOM | 2354 | N | PRO | A | 379 | −44.110 | −85.868 | 79.864 | 1.00 | 193.95 | N |
| ATOM | 2355 | CA | PRO | A | 379 | −44.017 | −84.727 | 80.781 | 1.00 | 194.05 | C |
| ATOM | 2356 | C | PRO | A | 379 | −43.601 | −85.137 | 82.192 | 1.00 | 194.20 | C |
| ATOM | 2357 | O | PRO | A | 379 | −44.093 | −86.136 | 82.719 | 1.00 | 194.25 | O |
| ATOM | 2358 | CB | PRO | A | 379 | −45.445 | −84.180 | 80.798 | 1.00 | 194.04 | C |
| ATOM | 2359 | CG | PRO | A | 379 | −46.328 | −85.348 | 80.373 | 1.00 | 193.93 | C |
| ATOM | 2360 | CD | PRO | A | 379 | −45.447 | −86.486 | 79.938 | 1.00 | 193.83 | C |
| ATOM | 2361 | N | LYS | A | 380 | −42.710 | −84.361 | 82.799 | 1.00 | 194.40 | N |
| ATOM | 2362 | CA | LYS | A | 380 | −42.096 | −84.751 | 84.068 | 1.00 | 194.74 | C |
| ATOM | 2363 | C | LYS | A | 380 | −43.049 | −84.555 | 85.246 | 1.00 | 194.94 | C |
| ATOM | 2364 | O | LYS | A | 380 | −44.150 | −84.006 | 85.093 | 1.00 | 194.89 | O |
| ATOM | 2365 | CB | LYS | A | 380 | −40.773 | −83.996 | 84.318 | 1.00 | 194.76 | C |
| ATOM | 2366 | CG | LYS | A | 380 | −40.081 | −83.423 | 83.071 | 1.00 | 194.96 | C |
| ATOM | 2367 | CD | LYS | A | 380 | −39.003 | −84.323 | 82.478 | 1.00 | 194.87 | C |
| ATOM | 2368 | CE | LYS | A | 380 | −38.641 | −83.867 | 81.061 | 1.00 | 194.38 | C |
| ATOM | 2369 | NZ | LYS | A | 380 | −37.184 | −83.976 | 80.781 | 1.00 | 193.98 | N |
| ATOM | 2370 | N | THR | A | 381 | −42.604 | −85.020 | 86.413 | 1.00 | 195.27 | N |
| ATOM | 2371 | CA | THR | A | 381 | −43.335 | −84.870 | 87.669 | 1.00 | 195.50 | C |
| ATOM | 2372 | C | THR | A | 381 | −42.330 | −84.664 | 88.842 | 1.00 | 195.70 | C |
| ATOM | 2373 | O | THR | A | 381 | −41.840 | −85.625 | 89.444 | 1.00 | 195.62 | O |
| ATOM | 2374 | CB | THR | A | 381 | −44.394 | −86.027 | 87.845 | 1.00 | 195.50 | C |
| ATOM | 2375 | OG1 | THR | A | 381 | −45.206 | −85.796 | 89.000 | 1.00 | 195.52 | O |
| ATOM | 2376 | CG2 | THR | A | 381 | −43.748 | −87.428 | 87.894 | 1.00 | 195.44 | C |
| ATOM | 2377 | N | TRP | A | 382 | −42.025 | −83.387 | 89.123 | 1.00 | 196.07 | N |
| ATOM | 2378 | CA | TRP | A | 382 | −40.924 | −82.953 | 90.025 | 1.00 | 196.40 | C |
| ATOM | 2379 | C | TRP | A | 382 | −41.264 | −83.016 | 91.511 | 1.00 | 196.38 | C |
| ATOM | 2380 | O | TRP | A | 382 | −42.428 | −82.954 | 91.890 | 1.00 | 196.55 | O |
| ATOM | 2381 | CB | TRP | A | 382 | −40.451 | −81.525 | 89.675 | 1.00 | 196.70 | C |
| ATOM | 2382 | CG | TRP | A | 382 | −39.824 | −81.410 | 88.303 | 1.00 | 197.29 | C |
| ATOM | 2383 | CD1 | TRP | A | 382 | −40.478 | −81.375 | 87.102 | 1.00 | 197.92 | C |
| ATOM | 2384 | CD2 | TRP | A | 382 | −38.427 | −81.324 | 87.993 | 1.00 | 197.75 | C |
| ATOM | 2385 | NE1 | TRP | A | 382 | −39.578 | −81.277 | 86.067 | 1.00 | 197.91 | N |
| ATOM | 2386 | CE2 | TRP | A | 382 | −38.312 | −81.248 | 86.586 | 1.00 | 197.86 | C |
| ATOM | 2387 | CE3 | TRP | A | 382 | −37.261 | −81.306 | 88.766 | 1.00 | 198.15 | C |
| ATOM | 2388 | CZ2 | TRP | A | 382 | −37.078 | −81.155 | 85.936 | 1.00 | 197.80 | C |
| ATOM | 2389 | CZ3 | TRP | A | 382 | −36.026 | −81.215 | 88.114 | 1.00 | 197.97 | C |
| ATOM | 2390 | CH2 | TRP | A | 382 | −35.949 | −81.141 | 86.715 | 1.00 | 197.67 | C |
| ATOM | 2391 | N | VAL | A | 383 | −40.244 | −83.132 | 92.356 | 1.00 | 196.32 | N |
| ATOM | 2392 | CA | VAL | A | 383 | −40.471 | −83.228 | 93.798 | 1.00 | 196.17 | C |
| ATOM | 2393 | C | VAL | A | 383 | −39.494 | −82.470 | 94.673 | 1.00 | 196.39 | C |
| ATOM | 2394 | O | VAL | A | 383 | −38.271 | −82.571 | 94.533 | 1.00 | 196.32 | O |
| ATOM | 2395 | CB | VAL | A | 383 | −40.552 | −84.675 | 94.295 | 1.00 | 196.01 | C |
| ATOM | 2396 | CG1 | VAL | A | 383 | −41.982 | −85.011 | 94.614 | 1.00 | 195.59 | C |
| ATOM | 2397 | CG2 | VAL | A | 383 | −39.915 | −85.659 | 93.285 | 1.00 | 195.97 | C |
| ATOM | 2398 | N | HIS | A | 384 | −40.071 | −81.708 | 95.592 | 1.00 | 196.73 | N |
| ATOM | 2399 | CA | HIS | A | 384 | −39.314 | −80.856 | 96.490 | 1.00 | 197.10 | C |
| ATOM | 2400 | C | HIS | A | 384 | −39.817 | −80.894 | 97.927 | 1.00 | 196.77 | C |
| ATOM | 2401 | O | HIS | A | 384 | −40.946 | −80.479 | 98.230 | 1.00 | 196.91 | O |
| ATOM | 2402 | CB | HIS | A | 384 | −39.337 | −79.424 | 95.988 | 1.00 | 197.35 | C |
| ATOM | 2403 | CG | HIS | A | 384 | −38.615 | −79.239 | 94.699 | 1.00 | 199.41 | C |
| ATOM | 2404 | ND1 | HIS | A | 384 | −37.346 | −78.708 | 94.629 | 1.00 | 201.50 | N |
| ATOM | 2405 | CD2 | HIS | A | 384 | −38.973 | −79.536 | 93.427 | 1.00 | 201.38 | C |
| ATOM | 2406 | CE1 | HIS | A | 384 | −36.959 | −78.666 | 93.365 | 1.00 | 202.72 | C |
| ATOM | 2407 | NE2 | HIS | A | 384 | −37.927 | −79.166 | 92.615 | 1.00 | 202.72 | N |
| ATOM | 2408 | N | TYR | A | 385 | −38.964 | −81.409 | 98.805 | 1.00 | 196.11 | N |
| ATOM | 2409 | CA | TYR | A | 385 | −39.164 | −81.265 | 100.223 | 1.00 | 195.11 | C |
| ATOM | 2410 | C | TYR | A | 385 | −38.325 | −80.064 | 100.669 | 1.00 | 194.39 | C |
| ATOM | 2411 | O | TYR | A | 385 | −37.095 | −80.147 | 100.739 | 1.00 | 194.04 | O |
| ATOM | 2412 | CB | TYR | A | 385 | −38.721 | −82.526 | 100.935 | 1.00 | 195.28 | C |
| ATOM | 2413 | CG | TYR | A | 385 | −39.390 | −83.811 | 100.487 | 1.00 | 195.50 | C |
| ATOM | 2414 | CD1 | TYR | A | 385 | −38.733 | −84.699 | 99.636 | 1.00 | 195.63 | C |
| ATOM | 2415 | CD2 | TYR | A | 385 | −40.657 | −84.168 | 100.956 | 1.00 | 195.83 | C |
| ATOM | 2416 | CE1 | TYR | A | 385 | −39.327 | −85.906 | 99.251 | 1.00 | 195.70 | C |
| ATOM | 2417 | CE2 | TYR | A | 385 | −41.257 | −85.376 | 100.579 | 1.00 | 195.80 | C |
| ATOM | 2418 | CZ | TYR | A | 385 | −40.586 | −86.234 | 99.726 | 1.00 | 195.61 | C |
| ATOM | 2419 | OH | TYR | A | 385 | −41.171 | −87.417 | 99.346 | 1.00 | 195.54 | O |
| ATOM | 2420 | N | ILE | A | 386 | −39.003 | −78.944 | 100.923 | 1.00 | 193.52 | N |
| ATOM | 2421 | CA | ILE | A | 386 | −38.372 | −77.708 | 101.386 | 1.00 | 192.60 | C |
| ATOM | 2422 | C | ILE | A | 386 | −38.918 | −77.324 | 102.748 | 1.00 | 192.29 | C |
| ATOM | 2423 | O | ILE | A | 386 | −40.114 | −77.465 | 103.012 | 1.00 | 192.21 | O |
| ATOM | 2424 | CB | ILE | A | 386 | −38.644 | −76.534 | 100.445 | 1.00 | 192.38 | C |
| ATOM | 2425 | CG1 | ILE | A | 386 | −38.274 | −76.901 | 99.024 | 1.00 | 192.11 | C |
| ATOM | 2426 | CG2 | ILE | A | 386 | −37.863 | −75.301 | 100.876 | 1.00 | 192.12 | C |
| ATOM | 2427 | CD1 | ILE | A | 386 | −38.755 | −75.898 | 98.043 | 1.00 | 192.83 | C |
| ATOM | 2428 | N | ALA | A | 387 | −38.027 | −76.836 | 103.602 | 1.00 | 191.75 | N |
| ATOM | 2429 | CA | ALA | A | 387 | −38.394 | −76.313 | 104.896 | 1.00 | 191.24 | C |
| ATOM | 2430 | C | ALA | A | 387 | −38.030 | −74.851 | 104.901 | 1.00 | 191.01 | C |
| ATOM | 2431 | O | ALA | A | 387 | −37.080 | −74.455 | 104.237 | 1.00 | 190.94 | O |
| ATOM | 2432 | CB | ALA | A | 387 | −37.633 | −77.037 | 105.977 | 1.00 | 191.29 | C |
| ATOM | 2433 | N | ALA | A | 388 | −38.797 | −74.042 | 105.619 | 1.00 | 190.93 | N |
| ATOM | 2434 | CA | ALA | A | 388 | −38.349 | −72.695 | 105.949 | 1.00 | 191.23 | C |
| ATOM | 2435 | C | ALA | A | 388 | −37.667 | −72.814 | 107.295 | 1.00 | 191.45 | C |
| ATOM | 2436 | O | ALA | A | 388 | −38.192 | −73.470 | 108.197 | 1.00 | 191.71 | O |
| ATOM | 2437 | CB | ALA | A | 388 | −39.506 | −71.730 | 106.015 | 1.00 | 191.20 | C |
| ATOM | 2438 | N | GLU | A | 389 | −36.498 | −72.201 | 107.439 | 1.00 | 191.58 | N |
| ATOM | 2439 | CA | GLU | A | 389 | −35.643 | −72.541 | 108.565 | 1.00 | 191.88 | C |
| ATOM | 2440 | C | GLU | A | 389 | −34.699 | −71.412 | 108.956 | 1.00 | 191.80 | C |
| ATOM | 2441 | O | GLU | A | 389 | −34.112 | −70.760 | 108.096 | 1.00 | 191.78 | O |
| ATOM | 2442 | CB | GLU | A | 389 | −34.905 | −73.842 | 108.235 | 1.00 | 191.93 | C |
| ATOM | 2443 | CG | GLU | A | 389 | −33.646 | −74.115 | 108.997 | 1.00 | 193.45 | C |
| ATOM | 2444 | CD | GLU | A | 389 | −32.665 | −74.930 | 108.176 | 1.00 | 195.72 | C |
| ATOM | 2445 | OE1 | GLU | A | 389 | −32.186 | −75.961 | 108.703 | 1.00 | 197.25 | O |
| ATOM | 2446 | OE2 | GLU | A | 389 | −32.379 | −74.546 | 107.008 | 1.00 | 195.75 | O |
| ATOM | 2447 | N | GLU | A | 390 | −34.580 | −71.189 | 110.263 | 1.00 | 191.87 | N |
| ATOM | 2448 | CA | GLU | A | 390 | −33.781 | −70.107 | 110.819 | 1.00 | 191.88 | C |
| ATOM | 2449 | C | GLU | A | 390 | −32.323 | −70.538 | 110.917 | 1.00 | 192.36 | C |
| ATOM | 2450 | O | GLU | A | 390 | −32.025 | −71.694 | 111.238 | 1.00 | 192.44 | O |
| ATOM | 2451 | CB | GLU | A | 390 | −34.315 | −69.709 | 112.198 | 1.00 | 191.87 | C |
| ATOM | 2452 | CG | GLU | A | 390 | −35.825 | −69.942 | 112.386 | 1.00 | 191.50 | C |
| ATOM | 2453 | CD | GLU | A | 390 | −36.419 | −69.342 | 113.671 | 1.00 | 191.47 | C |
| ATOM | 2454 | OE1 | GLU | A | 390 | −35.724 | −69.278 | 114.708 | 1.00 | 191.59 | O |
| ATOM | 2455 | OE2 | GLU | A | 390 | −37.603 | −68.944 | 113.652 | 1.00 | 190.07 | O |
| ATOM | 2456 | N | GLU | A | 391 | −31.426 | −69.600 | 110.635 | 1.00 | 192.87 | N |
| ATOM | 2457 | CA | GLU | A | 391 | −29.983 | −69.842 | 110.602 | 1.00 | 193.77 | C |
| ATOM | 2458 | C | GLU | A | 391 | −29.302 | −68.532 | 111.024 | 1.00 | 193.99 | C |
| ATOM | 2459 | O | GLU | A | 391 | −29.950 | −67.680 | 111.623 | 1.00 | 194.19 | O |
| ATOM | 2460 | CB | GLU | A | 391 | −29.567 | −70.253 | 109.181 | 1.00 | 193.69 | C |
| ATOM | 2461 | CG | GLU | A | 391 | −28.400 | −71.265 | 109.074 | 1.00 | 194.58 | C |
| ATOM | 2462 | CD | GLU | A | 391 | −27.924 | −71.537 | 107.616 | 1.00 | 194.66 | C |
| ATOM | 2463 | OE1 | GLU | A | 391 | −28.515 | −70.992 | 106.650 | 1.00 | 195.23 | O |
| ATOM | 2464 | OE2 | GLU | A | 391 | −26.941 | −72.304 | 107.442 | 1.00 | 195.83 | O |
| ATOM | 2465 | N | ASP | A | 392 | −28.010 | −68.372 | 110.734 | 1.00 | 194.42 | N |
| ATOM | 2466 | CA | ASP | A | 392 | −27.319 | −67.082 | 110.890 | 1.00 | 194.82 | C |
| ATOM | 2467 | C | ASP | A | 392 | −26.716 | −66.665 | 109.551 | 1.00 | 195.15 | C |
| ATOM | 2468 | O | ASP | A | 392 | −26.310 | −67.520 | 108.765 | 1.00 | 195.14 | O |
| ATOM | 2469 | CB | ASP | A | 392 | −26.200 | −67.170 | 111.940 | 1.00 | 194.83 | C |
| ATOM | 2470 | CG | ASP | A | 392 | −26.719 | −67.165 | 113.371 | 1.00 | 194.96 | C |
| ATOM | 2471 | OD1 | ASP | A | 392 | −27.483 | −68.083 | 113.727 | 1.00 | 195.88 | O |
| ATOM | 2472 | OD2 | ASP | A | 392 | −26.340 | −66.260 | 114.152 | 1.00 | 194.34 | O |
| ATOM | 2473 | N | TRP | A | 393 | −26.634 | −65.364 | 109.286 | 1.00 | 195.67 | N |
| ATOM | 2474 | CA | TRP | A | 393 | −26.022 | −64.917 | 108.036 | 1.00 | 196.22 | C |
| ATOM | 2475 | C | TRP | A | 393 | −24.877 | −63.923 | 108.170 | 1.00 | 196.34 | C |
| ATOM | 2476 | O | TRP | A | 393 | −25.080 | −62.802 | 108.627 | 1.00 | 196.21 | O |
| ATOM | 2477 | CB | TRP | A | 393 | −27.068 | −64.373 | 107.063 | 1.00 | 196.66 | C |
| ATOM | 2478 | CG | TRP | A | 393 | −26.841 | −64.831 | 105.647 | 1.00 | 197.26 | C |
| ATOM | 2479 | CD1 | TRP | A | 393 | −27.671 | −64.635 | 104.581 | 1.00 | 197.45 | C |
| ATOM | 2480 | CD2 | TRP | A | 393 | −25.729 | −65.602 | 105.151 | 1.00 | 198.40 | C |
| ATOM | 2481 | NE1 | TRP | A | 393 | −27.142 | −65.213 | 103.450 | 1.00 | 197.41 | N |
| ATOM | 2482 | CE2 | TRP | A | 393 | −25.954 | −65.815 | 103.769 | 1.00 | 198.20 | C |
| ATOM | 2483 | CE3 | TRP | A | 393 | −24.559 | −66.137 | 105.744 | 1.00 | 198.84 | C |
| ATOM | 2484 | CZ2 | TRP | A | 393 | −25.052 | −66.530 | 102.962 | 1.00 | 198.21 | C |
| ATOM | 2485 | CZ3 | TRP | A | 393 | −23.656 | −66.848 | 104.945 | 1.00 | 197.95 | C |
| ATOM | 2486 | CH2 | TRP | A | 393 | −23.912 | −67.038 | 103.567 | 1.00 | 198.13 | C |
| ATOM | 2487 | N | ASP | A | 394 | −23.681 | −64.346 | 107.759 | 1.00 | 196.75 | N |
| ATOM | 2488 | CA | ASP | A | 394 | −22.525 | −63.448 | 107.609 | 1.00 | 197.30 | C |
| ATOM | 2489 | C | ASP | A | 394 | −22.453 | −62.985 | 106.161 | 1.00 | 197.53 | C |
| ATOM | 2490 | O | ASP | A | 394 | −21.992 | −63.727 | 105.289 | 1.00 | 197.58 | O |
| ATOM | 2491 | CB | ASP | A | 394 | −21.203 | −64.142 | 108.028 | 1.00 | 197.42 | C |
| ATOM | 2492 | CG | ASP | A | 394 | −19.999 | −63.160 | 108.162 | 1.00 | 197.37 | C |
| ATOM | 2493 | OD1 | ASP | A | 394 | −19.965 | −62.121 | 107.459 | 1.00 | 196.96 | O |
| ATOM | 2494 | OD2 | ASP | A | 394 | −19.073 | −63.447 | 108.970 | 1.00 | 196.85 | O |
| ATOM | 2495 | N | TYR | A | 395 | −22.910 | −61.760 | 105.913 | 1.00 | 197.87 | N |
| ATOM | 2496 | CA | TYR | A | 395 | −22.931 | −61.200 | 104.559 | 1.00 | 198.34 | C |
| ATOM | 2497 | C | TYR | A | 395 | −21.532 | −61.079 | 103.929 | 1.00 | 199.05 | C |
| ATOM | 2498 | O | TYR | A | 395 | −21.397 | −61.174 | 102.707 | 1.00 | 199.12 | O |
| ATOM | 2499 | CB | TYR | A | 395 | −23.646 | −59.830 | 104.510 | 1.00 | 197.83 | C |
| ATOM | 2500 | CG | TYR | A | 395 | −25.150 | −59.820 | 104.782 | 1.00 | 197.13 | C |
| ATOM | 2501 | CD1 | TYR | A | 395 | −25.999 | −60.800 | 104.258 | 1.00 | 196.72 | C |
| ATOM | 2502 | CD2 | TYR | A | 395 | −25.725 | −58.799 | 105.535 | 1.00 | 196.25 | C |
| ATOM | 2503 | CE1 | TYR | A | 395 | −27.374 | −60.771 | 104.502 | 1.00 | 196.14 | C |
| ATOM | 2504 | CE2 | TYR | A | 395 | −27.093 | −58.771 | 105.787 | 1.00 | 195.86 | C |
| ATOM | 2505 | CZ | TYR | A | 395 | −27.909 | −59.753 | 105.267 | 1.00 | 196.03 | C |
| ATOM | 2506 | OH | TYR | A | 395 | −29.256 | −59.717 | 105.524 | 1.00 | 195.92 | O |
| ATOM | 2507 | N | ALA | A | 396 | −20.504 | −60.867 | 104.755 | 1.00 | 199.99 | N |
| ATOM | 2508 | CA | ALA | A | 396 | −19.140 | −60.605 | 104.256 | 1.00 | 200.91 | C |
| ATOM | 2509 | C | ALA | A | 396 | −18.051 | −61.396 | 104.997 | 1.00 | 201.51 | C |
| ATOM | 2510 | O | ALA | A | 396 | −17.217 | −60.815 | 105.707 | 1.00 | 201.61 | O |
| ATOM | 2511 | CB | ALA | A | 396 | −18.835 | −59.095 | 104.273 | 1.00 | 200.88 | C |
| ATOM | 2512 | N | PRO | A | 397 | −18.043 | −62.726 | 104.816 | 1.00 | 202.09 | N |
| ATOM | 2513 | CA | PRO | A | 397 | −17.157 | −63.576 | 105.594 | 1.00 | 202.72 | C |
| ATOM | 2514 | C | PRO | A | 397 | −15.721 | −63.518 | 105.085 | 1.00 | 203.34 | C |
| ATOM | 2515 | O | PRO | A | 397 | −14.855 | −62.947 | 105.756 | 1.00 | 203.27 | O |
| ATOM | 2516 | CB | PRO | A | 397 | −17.749 | −64.980 | 105.393 | 1.00 | 202.81 | C |
| ATOM | 2517 | CG | PRO | A | 397 | −19.010 | −64.794 | 104.568 | 1.00 | 202.38 | C |
| ATOM | 2518 | CD | PRO | A | 397 | −18.849 | −63.509 | 103.867 | 1.00 | 201.99 | C |
| ATOM | 2519 | N | LEU | A | 398 | −15.497 | −64.104 | 103.906 | 1.00 | 204.27 | N |
| ATOM | 2520 | CA | LEU | A | 398 | −14.188 | −64.150 | 103.236 | 1.00 | 204.81 | C |
| ATOM | 2521 | C | LEU | A | 398 | −13.389 | −62.866 | 103.523 | 1.00 | 205.04 | C |
| ATOM | 2522 | O | LEU | A | 398 | −12.340 | −62.916 | 104.184 | 1.00 | 204.99 | O |
| ATOM | 2523 | CB | LEU | A | 398 | −14.380 | −64.403 | 101.711 | 1.00 | 205.11 | C |
| ATOM | 2524 | CG | LEU | A | 398 | −13.337 | −64.853 | 100.643 | 1.00 | 205.05 | C |
| ATOM | 2525 | CD1 | LEU | A | 398 | −12.637 | −63.679 | 99.904 | 1.00 | 205.38 | C |
| ATOM | 2526 | CD2 | LEU | A | 398 | −12.306 | −65.898 | 101.143 | 1.00 | 205.52 | C |
| ATOM | 2527 | N | VAL | A | 399 | −13.922 | −61.726 | 103.065 | 1.00 | 205.16 | N |
| ATOM | 2528 | CA | VAL | A | 399 | −13.262 | −60.421 | 103.202 | 1.00 | 205.03 | C |
| ATOM | 2529 | C | VAL | A | 399 | −13.887 | −59.567 | 104.320 | 1.00 | 204.93 | C |
| ATOM | 2530 | O | VAL | A | 399 | −15.113 | −59.505 | 104.467 | 1.00 | 204.71 | O |
| ATOM | 2531 | CB | VAL | A | 399 | −13.161 | −59.658 | 101.819 | 1.00 | 205.01 | C |
| ATOM | 2532 | CG1 | VAL | A | 399 | −14.535 | −59.303 | 101.262 | 1.00 | 204.88 | C |
| ATOM | 2533 | CG2 | VAL | A | 399 | −12.253 | −58.423 | 101.915 | 1.00 | 204.93 | C |
| ATOM | 2534 | N | LEU | A | 400 | −13.012 | −58.952 | 105.118 | 1.00 | 204.93 | N |
| ATOM | 2535 | CA | LEU | A | 400 | −13.384 | −58.004 | 106.173 | 1.00 | 204.90 | C |
| ATOM | 2536 | C | LEU | A | 400 | −12.197 | −57.079 | 106.493 | 1.00 | 204.85 | C |
| ATOM | 2537 | O | LEU | A | 400 | −11.064 | −57.546 | 106.606 | 1.00 | 204.80 | O |
| ATOM | 2538 | CB | LEU | A | 400 | −13.897 | −58.735 | 107.435 | 1.00 | 204.88 | C |
| ATOM | 2539 | CG | LEU | A | 400 | −13.041 | −59.707 | 108.267 | 1.00 | 204.70 | C |
| ATOM | 2540 | CD1 | LEU | A | 400 | −13.669 | −59.931 | 109.643 | 1.00 | 204.49 | C |
| ATOM | 2541 | CD2 | LEU | A | 400 | −12.815 | −61.040 | 107.560 | 1.00 | 204.25 | C |
| ATOM | 2542 | N | ALA | A | 401 | −12.451 | −55.773 | 106.604 | 1.00 | 204.83 | N |
| ATOM | 2543 | CA | ALA | A | 401 | −11.406 | −54.798 | 106.972 | 1.00 | 204.88 | C |
| ATOM | 2544 | C | ALA | A | 401 | −11.935 | −53.452 | 107.527 | 1.00 | 204.95 | C |
| ATOM | 2545 | O | ALA | A | 401 | −11.421 | −52.383 | 107.157 | 1.00 | 204.85 | O |
| ATOM | 2546 | CB | ALA | A | 401 | −10.453 | −54.561 | 105.793 | 1.00 | 204.85 | C |
| ATOM | 2547 | N | PRO | A | 402 | −12.935 | −53.499 | 108.443 | 1.00 | 205.03 | N |
| ATOM | 2548 | CA | PRO | A | 402 | −13.609 | −52.277 | 108.911 | 1.00 | 205.00 | C |
| ATOM | 2549 | C | PRO | A | 402 | −13.004 | −51.564 | 110.142 | 1.00 | 204.92 | C |
| ATOM | 2550 | O | PRO | A | 402 | −12.139 | −52.116 | 110.837 | 1.00 | 204.96 | O |
| ATOM | 2551 | CB | PRO | A | 402 | −15.013 | −52.788 | 109.262 | 1.00 | 205.02 | C |
| ATOM | 2552 | CG | PRO | A | 402 | −14.791 | −54.205 | 109.718 | 1.00 | 204.95 | C |
| ATOM | 2553 | CD | PRO | A | 402 | −13.502 | −54.701 | 109.094 | 1.00 | 205.04 | C |
| ATOM | 2554 | N | ASP | A | 403 | −13.466 | −50.329 | 110.365 | 1.00 | 204.69 | N |
| ATOM | 2555 | CA | ASP | A | 403 | −13.345 | −49.604 | 111.644 | 1.00 | 204.31 | C |
| ATOM | 2556 | C | ASP | A | 403 | −14.412 | −48.487 | 111.676 | 1.00 | 204.01 | C |
| ATOM | 2557 | O | ASP | A | 403 | −15.605 | −48.781 | 111.818 | 1.00 | 204.10 | O |
| ATOM | 2558 | CB | ASP | A | 403 | −11.927 | −49.064 | 111.869 | 1.00 | 204.29 | C |
| ATOM | 2559 | CG | ASP | A | 403 | −11.475 | −49.205 | 113.316 | 1.00 | 204.27 | C |
| ATOM | 2560 | OD1 | ASP | A | 403 | −11.915 | −50.164 | 113.992 | 1.00 | 204.06 | O |
| ATOM | 2561 | OD2 | ASP | A | 403 | −10.675 | −48.364 | 113.780 | 1.00 | 204.37 | O |
| ATOM | 2562 | N | ASP | A | 404 | −13.991 | −47.225 | 111.565 | 1.00 | 203.44 | N |
| ATOM | 2563 | CA | ASP | A | 404 | −14.872 | −46.148 | 111.094 | 1.00 | 202.88 | C |
| ATOM | 2564 | C | ASP | A | 404 | −14.277 | −45.715 | 109.765 | 1.00 | 202.43 | C |
| ATOM | 2565 | O | ASP | A | 404 | −14.599 | −44.660 | 109.216 | 1.00 | 202.21 | O |
| ATOM | 2566 | CB | ASP | A | 404 | −14.977 | −44.987 | 112.100 | 1.00 | 202.94 | C |
| ATOM | 2567 | CG | ASP | A | 404 | −13.640 | −44.305 | 112.379 | 1.00 | 203.17 | C |
| ATOM | 2568 | OD1 | ASP | A | 404 | −13.651 | −43.200 | 112.971 | 1.00 | 203.21 | O |
| ATOM | 2569 | OD2 | ASP | A | 404 | −12.581 | −44.862 | 112.020 | 1.00 | 203.60 | O |
| ATOM | 2570 | N | ARG | A | 405 | −13.426 | −46.609 | 109.263 | 1.00 | 202.06 | N |
| ATOM | 2571 | CA | ARG | A | 405 | −12.523 | −46.400 | 108.142 | 1.00 | 201.82 | C |
| ATOM | 2572 | C | ARG | A | 405 | −13.224 | −46.158 | 106.805 | 1.00 | 201.82 | C |
| ATOM | 2573 | O | ARG | A | 405 | −12.583 | −45.730 | 105.842 | 1.00 | 201.77 | O |
| ATOM | 2574 | CB | ARG | A | 405 | −11.592 | −47.619 | 108.025 | 1.00 | 201.69 | C |
| ATOM | 2575 | CG | ARG | A | 405 | −10.108 | −47.289 | 107.939 | 1.00 | 201.15 | C |
| ATOM | 2576 | CD | ARG | A | 405 | −9.648 | −46.997 | 106.517 | 1.00 | 200.18 | C |
| ATOM | 2577 | NE | ARG | A | 405 | −8.517 | −46.069 | 106.504 | 1.00 | 199.39 | N |
| ATOM | 2578 | CZ | ARG | A | 405 | −8.561 | −44.821 | 106.043 | 1.00 | 198.68 | C |
| ATOM | 2579 | NH1 | ARG | A | 405 | −9.679 | −44.333 | 105.525 | 1.00 | 198.33 | N |
| ATOM | 2580 | NH2 | ARG | A | 405 | −7.476 | −44.060 | 106.087 | 1.00 | 198.35 | N |
| ATOM | 2581 | N | SER | A | 406 | −14.529 | −46.430 | 106.743 | 1.00 | 201.83 | N |
| ATOM | 2582 | CA | SER | A | 406 | −15.283 | −46.357 | 105.479 | 1.00 | 201.92 | C |
| ATOM | 2583 | C | SER | A | 406 | −16.806 | −46.225 | 105.644 | 1.00 | 201.83 | C |
| ATOM | 2584 | O | SER | A | 406 | −17.322 | −46.160 | 106.766 | 1.00 | 201.85 | O |
| ATOM | 2585 | CB | SER | A | 406 | −14.978 | −47.585 | 104.601 | 1.00 | 201.99 | C |
| ATOM | 2586 | OG | SER | A | 406 | −13.614 | −47.635 | 104.211 | 1.00 | 202.19 | O |
| ATOM | 2587 | N | TYR | A | 407 | −17.506 | −46.171 | 104.506 | 1.00 | 201.67 | N |
| ATOM | 2588 | CA | TYR | A | 407 | −18.957 | −46.329 | 104.462 | 1.00 | 201.51 | C |
| ATOM | 2589 | C | TYR | A | 407 | −19.291 | −47.784 | 104.688 | 1.00 | 201.36 | C |
| ATOM | 2590 | O | TYR | A | 407 | −20.339 | −48.093 | 105.243 | 1.00 | 201.34 | O |
| ATOM | 2591 | CB | TYR | A | 407 | −19.554 | −45.871 | 103.125 | 1.00 | 201.57 | C |
| ATOM | 2592 | CG | TYR | A | 407 | −20.043 | −44.431 | 103.099 | 1.00 | 201.65 | C |
| ATOM | 2593 | CD1 | TYR | A | 407 | −20.204 | −43.703 | 104.285 | 1.00 | 201.80 | C |
| ATOM | 2594 | CD2 | TYR | A | 407 | −20.373 | −43.806 | 101.888 | 1.00 | 201.38 | C |
| ATOM | 2595 | CE1 | TYR | A | 407 | −20.652 | −42.388 | 104.266 | 1.00 | 201.74 | C |
| ATOM | 2596 | CE2 | TYR | A | 407 | −20.830 | −42.490 | 101.858 | 1.00 | 201.31 | C |
| ATOM | 2597 | CZ | TYR | A | 407 | −20.965 | −41.789 | 103.053 | 1.00 | 201.62 | C |
| ATOM | 2598 | OH | TYR | A | 407 | −21.410 | −40.489 | 103.053 | 1.00 | 201.53 | O |
| ATOM | 2599 | N | LYS | A | 408 | −18.395 | −48.670 | 104.248 | 1.00 | 201.21 | N |
| ATOM | 2600 | CA | LYS | A | 408 | −18.503 | −50.098 | 104.552 | 1.00 | 201.19 | C |
| ATOM | 2601 | C | LYS | A | 408 | −18.211 | −50.380 | 106.033 | 1.00 | 201.19 | C |
| ATOM | 2602 | O | LYS | A | 408 | −18.798 | −51.288 | 106.629 | 1.00 | 201.12 | O |
| ATOM | 2603 | CB | LYS | A | 408 | −17.596 | −50.947 | 103.642 | 1.00 | 201.13 | C |
| ATOM | 2604 | CG | LYS | A | 408 | −16.132 | −51.049 | 104.062 | 1.00 | 200.90 | C |
| ATOM | 2605 | CD | LYS | A | 408 | −15.558 | −52.449 | 103.840 | 1.00 | 200.27 | C |
| ATOM | 2606 | CE | LYS | A | 408 | −15.017 | −52.630 | 102.430 | 1.00 | 200.16 | C |
| ATOM | 2607 | NZ | LYS | A | 408 | −14.100 | −53.799 | 102.335 | 1.00 | 199.95 | N |
| ATOM | 2608 | N | SER | A | 409 | −17.309 | −49.588 | 106.616 | 1.00 | 201.18 | N |
| ATOM | 2609 | CA | SER | A | 409 | −16.938 | −49.723 | 108.022 | 1.00 | 201.13 | C |
| ATOM | 2610 | C | SER | A | 409 | −18.142 | −49.510 | 108.917 | 1.00 | 201.15 | C |
| ATOM | 2611 | O | SER | A | 409 | −18.223 | −50.098 | 109.990 | 1.00 | 201.17 | O |
| ATOM | 2612 | CB | SER | A | 409 | −15.846 | −48.723 | 108.401 | 1.00 | 201.12 | C |
| ATOM | 2613 | OG | SER | A | 409 | −14.583 | −49.099 | 107.882 | 1.00 | 201.04 | O |
| ATOM | 2614 | N | GLN | A | 410 | −19.068 | −48.671 | 108.458 | 1.00 | 201.13 | N |
| ATOM | 2615 | CA | GLN | A | 410 | −20.279 | −48.337 | 109.203 | 1.00 | 201.21 | C |
| ATOM | 2616 | C | GLN | A | 410 | −21.335 | −49.449 | 109.254 | 1.00 | 201.03 | C |
| ATOM | 2617 | O | GLN | A | 410 | −22.159 | −49.464 | 110.175 | 1.00 | 201.15 | O |
| ATOM | 2618 | CB | GLN | A | 410 | −20.911 | −47.051 | 108.654 | 1.00 | 201.21 | C |
| ATOM | 2619 | CG | GLN | A | 410 | −20.446 | −45.774 | 109.342 | 1.00 | 201.47 | C |
| ATOM | 2620 | CD | GLN | A | 410 | −21.369 | −44.596 | 109.067 | 1.00 | 201.57 | C |
| ATOM | 2621 | OE1 | GLN | A | 410 | −21.432 | −44.081 | 107.946 | 1.00 | 202.07 | O |
| ATOM | 2622 | NE2 | GLN | A | 410 | −22.090 | −44.161 | 110.097 | 1.00 | 201.89 | N |
| ATOM | 2623 | N | TYR | A | 411 | −21.309 | −50.372 | 108.289 | 1.00 | 200.76 | N |
| ATOM | 2624 | CA | TYR | A | 411 | −22.386 | −51.377 | 108.137 | 1.00 | 200.50 | C |
| ATOM | 2625 | C | TYR | A | 411 | −22.444 | −52.446 | 109.256 | 1.00 | 200.42 | C |
| ATOM | 2626 | O | TYR | A | 411 | −21.409 | −52.811 | 109.827 | 1.00 | 200.49 | O |
| ATOM | 2627 | CB | TYR | A | 411 | −22.402 | −51.968 | 106.708 | 1.00 | 200.30 | C |
| ATOM | 2628 | CG | TYR | A | 411 | −23.097 | −51.052 | 105.707 | 1.00 | 200.04 | C |
| ATOM | 2629 | CD1 | TYR | A | 411 | −22.443 | −49.942 | 105.173 | 1.00 | 199.73 | C |
| ATOM | 2630 | CD2 | TYR | A | 411 | −24.417 | −51.276 | 105.326 | 1.00 | 199.73 | C |
| ATOM | 2631 | CE1 | TYR | A | 411 | −23.078 | −49.088 | 104.284 | 1.00 | 199.36 | C |
| ATOM | 2632 | CE2 | TYR | A | 411 | −25.059 | −50.427 | 104.434 | 1.00 | 199.43 | C |
| ATOM | 2633 | CZ | TYR | A | 411 | −24.381 | −49.339 | 103.920 | 1.00 | 199.57 | C |
| ATOM | 2634 | OH | TYR | A | 411 | −25.011 | −48.498 | 103.040 | 1.00 | 199.99 | O |
| ATOM | 2635 | N | LEU | A | 412 | −23.664 | −52.918 | 109.555 | 1.00 | 200.14 | N |
| ATOM | 2636 | CA | LEU | A | 412 | −24.000 | −53.745 | 110.751 | 1.00 | 199.93 | C |
| ATOM | 2637 | C | LEU | A | 412 | −24.022 | −53.011 | 112.114 | 1.00 | 199.66 | C |
| ATOM | 2638 | O | LEU | A | 412 | −23.048 | −52.346 | 112.506 | 1.00 | 199.67 | O |
| ATOM | 2639 | CB | LEU | A | 412 | −23.142 | −55.023 | 110.856 | 1.00 | 199.95 | C |
| ATOM | 2640 | CG | LEU | A | 412 | −23.194 | −55.913 | 112.109 | 1.00 | 199.93 | C |
| ATOM | 2641 | CD1 | LEU | A | 412 | −24.032 | −57.139 | 111.875 | 1.00 | 199.53 | C |
| ATOM | 2642 | CD2 | LEU | A | 412 | −21.806 | −56.321 | 112.559 | 1.00 | 199.72 | C |
| ATOM | 2643 | N | ASN | A | 413 | −25.160 | −53.140 | 112.804 | 1.00 | 199.10 | N |
| ATOM | 2644 | CA | ASN | A | 413 | −25.339 | −52.775 | 114.223 | 1.00 | 198.33 | C |
| ATOM | 2645 | C | ASN | A | 413 | −24.637 | −51.496 | 114.714 | 1.00 | 198.13 | C |
| ATOM | 2646 | O | ASN | A | 413 | −23.612 | −51.560 | 115.399 | 1.00 | 198.06 | O |
| ATOM | 2647 | CB | ASN | A | 413 | −24.973 | −53.980 | 115.100 | 1.00 | 198.09 | C |
| ATOM | 2648 | CG | ASN | A | 413 | −25.394 | −55.303 | 114.480 | 1.00 | 196.90 | C |
| ATOM | 2649 | OD1 | ASN | A | 413 | −26.395 | −55.388 | 113.770 | 1.00 | 195.05 | O |
| ATOM | 2650 | ND2 | ASN | A | 413 | −24.617 | −56.340 | 114.740 | 1.00 | 195.86 | N |
| ATOM | 2651 | N | ASN | A | 414 | −25.223 | −50.343 | 114.379 | 1.00 | 197.83 | N |
| ATOM | 2652 | CA | ASN | A | 414 | −24.624 | −49.027 | 114.642 | 1.00 | 197.64 | C |
| ATOM | 2653 | C | ASN | A | 414 | −24.673 | −48.587 | 116.129 | 1.00 | 197.69 | C |
| ATOM | 2654 | O | ASN | A | 414 | −25.255 | −47.551 | 116.482 | 1.00 | 197.70 | O |
| ATOM | 2655 | CB | ASN | A | 414 | −25.237 | −47.963 | 113.705 | 1.00 | 197.47 | C |
| ATOM | 2656 | CG | ASN | A | 414 | −24.184 | −47.074 | 113.030 | 1.00 | 196.85 | C |
| ATOM | 2657 | OD1 | ASN | A | 414 | −23.087 | −47.523 | 112.697 | 1.00 | 196.10 | O |
| ATOM | 2658 | ND2 | ASN | A | 414 | −24.533 | −45.814 | 112.807 | 1.00 | 195.96 | N |
| ATOM | 2659 | N | GLY | A | 415 | −24.064 | −49.405 | 116.988 | 1.00 | 197.65 | N |
| ATOM | 2660 | CA | GLY | A | 415 | −23.748 | −49.036 | 118.365 | 1.00 | 197.55 | C |
| ATOM | 2661 | C | GLY | A | 415 | −22.266 | −49.316 | 118.523 | 1.00 | 197.58 | C |
| ATOM | 2662 | O | GLY | A | 415 | −21.444 | −48.618 | 117.927 | 1.00 | 197.45 | O |
| ATOM | 2663 | N | PRO | A | 416 | −21.907 | −50.318 | 119.347 | 1.00 | 197.68 | N |
| ATOM | 2664 | CA | PRO | A | 416 | −20.559 | −50.897 | 119.264 | 1.00 | 197.77 | C |
| ATOM | 2665 | C | PRO | A | 416 | −20.266 | −51.804 | 118.028 | 1.00 | 197.86 | C |
| ATOM | 2666 | O | PRO | A | 416 | −19.465 | −51.410 | 117.177 | 1.00 | 197.70 | O |
| ATOM | 2667 | CB | PRO | A | 416 | −20.417 | −51.662 | 120.597 | 1.00 | 197.74 | C |
| ATOM | 2668 | CG | PRO | A | 416 | −21.587 | −51.205 | 121.458 | 1.00 | 197.57 | C |
| ATOM | 2669 | CD | PRO | A | 416 | −22.667 | −50.883 | 120.478 | 1.00 | 197.67 | C |
| ATOM | 2670 | N | GLN | A | 417 | −20.906 | −52.977 | 117.928 | 1.00 | 198.06 | N |
| ATOM | 2671 | CA | GLN | A | 417 | −20.485 | −54.052 | 116.989 | 1.00 | 198.28 | C |
| ATOM | 2672 | C | GLN | A | 417 | −20.668 | −53.733 | 115.508 | 1.00 | 198.18 | C |
| ATOM | 2673 | O | GLN | A | 417 | −21.762 | −53.357 | 115.096 | 1.00 | 198.17 | O |
| ATOM | 2674 | CB | GLN | A | 417 | −21.185 | −55.378 | 117.319 | 1.00 | 198.40 | C |
| ATOM | 2675 | CG | GLN | A | 417 | −20.495 | −56.608 | 116.724 | 1.00 | 199.32 | C |
| ATOM | 2676 | CD | GLN | A | 417 | −19.035 | −56.739 | 117.158 | 1.00 | 201.03 | C |
| ATOM | 2677 | OE1 | GLN | A | 417 | −18.735 | −56.959 | 118.339 | 1.00 | 201.60 | O |
| ATOM | 2678 | NE2 | GLN | A | 417 | −18.118 | −56.601 | 116.198 | 1.00 | 201.63 | N |
| ATOM | 2679 | N | ARG | A | 418 | −19.611 | −53.950 | 114.710 | 1.00 | 198.17 | N |
| ATOM | 2680 | CA | ARG | A | 418 | −19.492 | −53.333 | 113.366 | 1.00 | 198.05 | C |
| ATOM | 2681 | C | ARG | A | 418 | −18.471 | −53.958 | 112.365 | 1.00 | 197.76 | C |
| ATOM | 2682 | O | ARG | A | 418 | −18.208 | −53.382 | 111.300 | 1.00 | 197.52 | O |
| ATOM | 2683 | CB | ARG | A | 418 | −19.170 | −51.848 | 113.574 | 1.00 | 198.21 | C |
| ATOM | 2684 | CG | ARG | A | 418 | −19.599 | −50.866 | 112.492 | 1.00 | 198.23 | C |
| ATOM | 2685 | CD | ARG | A | 418 | −19.172 | −49.444 | 112.898 | 1.00 | 198.16 | C |
| ATOM | 2686 | NE | ARG | A | 418 | −18.131 | −49.475 | 113.931 | 1.00 | 198.24 | N |
| ATOM | 2687 | CZ | ARG | A | 418 | −17.513 | −48.411 | 114.436 | 1.00 | 198.10 | C |
| ATOM | 2688 | NH1 | ARG | A | 418 | −17.803 | −47.190 | 114.004 | 1.00 | 198.36 | N |
| ATOM | 2689 | NH2 | ARG | A | 418 | −16.592 | −48.577 | 115.377 | 1.00 | 197.85 | N |
| ATOM | 2690 | N | ILE | A | 419 | −17.909 | −55.121 | 112.700 | 1.00 | 197.53 | N |
| ATOM | 2691 | CA | ILE | A | 419 | −16.927 | −55.814 | 111.835 | 1.00 | 197.17 | C |
| ATOM | 2692 | C | ILE | A | 419 | −17.579 | −56.419 | 110.561 | 1.00 | 196.94 | C |
| ATOM | 2693 | O | ILE | A | 419 | −18.499 | −55.818 | 109.994 | 1.00 | 196.81 | O |
| ATOM | 2694 | CB | ILE | A | 419 | −16.082 | −56.878 | 112.633 | 1.00 | 197.22 | C |
| ATOM | 2695 | CG1 | ILE | A | 419 | −16.975 | −57.986 | 113.219 | 1.00 | 196.85 | C |
| ATOM | 2696 | CG2 | ILE | A | 419 | −15.240 | −56.197 | 113.734 | 1.00 | 197.10 | C |
| ATOM | 2697 | CD1 | ILE | A | 419 | −16.369 | −59.373 | 113.145 | 1.00 | 196.00 | C |
| ATOM | 2698 | N | GLY | A | 420 | −17.090 | −57.579 | 110.105 | 1.00 | 196.62 | N |
| ATOM | 2699 | CA | GLY | A | 420 | −17.791 | −58.382 | 109.091 | 1.00 | 196.19 | C |
| ATOM | 2700 | C | GLY | A | 420 | −19.146 | −58.773 | 109.661 | 1.00 | 195.95 | C |
| ATOM | 2701 | O | GLY | A | 420 | −19.213 | −59.301 | 110.772 | 1.00 | 196.18 | O |
| ATOM | 2702 | N | ARG | A | 421 | −20.214 | −58.534 | 108.897 | 1.00 | 195.50 | N |
| ATOM | 2703 | CA | ARG | A | 421 | −21.585 | −58.384 | 109.442 | 1.00 | 195.06 | C |
| ATOM | 2704 | C | ARG | A | 421 | −22.477 | −59.624 | 109.555 | 1.00 | 194.69 | C |
| ATOM | 2705 | O | ARG | A | 421 | −22.819 | −60.240 | 108.542 | 1.00 | 194.60 | O |
| ATOM | 2706 | CB | ARG | A | 421 | −22.321 | −57.316 | 108.643 | 1.00 | 195.11 | C |
| ATOM | 2707 | CG | ARG | A | 421 | −21.685 | −55.962 | 108.775 | 1.00 | 195.40 | C |
| ATOM | 2708 | CD | ARG | A | 421 | −21.831 | −55.131 | 107.525 | 1.00 | 196.08 | C |
| ATOM | 2709 | NE | ARG | A | 421 | −20.562 | −54.978 | 106.816 | 1.00 | 196.43 | N |
| ATOM | 2710 | CZ | ARG | A | 421 | −19.530 | −54.256 | 107.248 | 1.00 | 196.74 | C |
| ATOM | 2711 | NH1 | ARG | A | 421 | −19.577 | −53.613 | 108.409 | 1.00 | 196.74 | N |
| ATOM | 2712 | NH2 | ARG | A | 421 | −18.431 | −54.185 | 106.514 | 1.00 | 197.20 | N |
| ATOM | 2713 | N | LYS | A | 422 | −22.888 | −59.939 | 110.789 | 1.00 | 194.23 | N |
| ATOM | 2714 | CA | LYS | A | 422 | −23.628 | −61.173 | 111.111 | 1.00 | 193.73 | C |
| ATOM | 2715 | C | LYS | A | 422 | −25.034 | −60.901 | 111.649 | 1.00 | 193.37 | C |
| ATOM | 2716 | O | LYS | A | 422 | −25.191 | −60.224 | 112.663 | 1.00 | 193.32 | O |
| ATOM | 2717 | CB | LYS | A | 422 | −22.830 | −62.026 | 112.119 | 1.00 | 193.70 | C |
| ATOM | 2718 | CG | LYS | A | 422 | −23.304 | −63.479 | 112.263 | 1.00 | 193.76 | C |
| ATOM | 2719 | CD | LYS | A | 422 | −22.514 | −64.274 | 113.315 | 1.00 | 193.70 | C |
| ATOM | 2720 | CE | LYS | A | 422 | −23.072 | −65.703 | 113.463 | 1.00 | 193.56 | C |
| ATOM | 2721 | NZ | LYS | A | 422 | −22.578 | −66.463 | 114.657 | 1.00 | 193.14 | N |
| ATOM | 2722 | N | TYR | A | 423 | −26.045 | −61.439 | 110.968 | 1.00 | 193.06 | N |
| ATOM | 2723 | CA | TYR | A | 423 | −27.444 | −61.323 | 111.398 | 1.00 | 193.06 | C |
| ATOM | 2724 | C | TYR | A | 423 | −28.137 | −62.698 | 111.429 | 1.00 | 193.25 | C |
| ATOM | 2725 | O | TYR | A | 423 | −27.677 | −63.642 | 110.780 | 1.00 | 193.29 | O |
| ATOM | 2726 | CB | TYR | A | 423 | −28.235 | −60.418 | 110.451 | 1.00 | 192.99 | C |
| ATOM | 2727 | CG | TYR | A | 423 | −27.794 | −58.974 | 110.328 | 1.00 | 192.84 | C |
| ATOM | 2728 | CD1 | TYR | A | 423 | −26.617 | −58.631 | 109.652 | 1.00 | 193.09 | C |
| ATOM | 2729 | CD2 | TYR | A | 423 | −28.588 | −57.943 | 110.828 | 1.00 | 192.52 | C |
| ATOM | 2730 | CE1 | TYR | A | 423 | −26.222 | −57.297 | 109.514 | 1.00 | 193.14 | C |
| ATOM | 2731 | CE2 | TYR | A | 423 | −28.203 | −56.606 | 110.696 | 1.00 | 192.75 | C |
| ATOM | 2732 | CZ | TYR | A | 423 | −27.019 | −56.291 | 110.041 | 1.00 | 193.02 | C |
| ATOM | 2733 | OH | TYR | A | 423 | −26.634 | −54.975 | 109.909 | 1.00 | 192.83 | O |
| ATOM | 2734 | N | LYS | A | 424 | −29.250 | −62.802 | 112.161 | 1.00 | 193.39 | N |
| ATOM | 2735 | CA | LYS | A | 424 | −30.039 | −64.037 | 112.188 | 1.00 | 193.54 | C |
| ATOM | 2736 | C | LYS | A | 424 | −31.186 | −63.973 | 111.193 | 1.00 | 193.67 | C |
| ATOM | 2737 | O | LYS | A | 424 | −32.121 | −63.180 | 111.348 | 1.00 | 193.38 | O |
| ATOM | 2738 | CB | LYS | A | 424 | −30.568 | −64.355 | 113.591 | 1.00 | 193.53 | C |
| ATOM | 2739 | CG | LYS | A | 424 | −30.929 | −65.839 | 113.778 | 1.00 | 193.67 | C |
| ATOM | 2740 | CD | LYS | A | 424 | −31.640 | −66.168 | 115.097 | 1.00 | 193.74 | C |
| ATOM | 2741 | CE | LYS | A | 424 | −30.727 | −66.043 | 116.327 | 1.00 | 194.36 | C |
| ATOM | 2742 | NZ | LYS | A | 424 | −29.579 | −66.997 | 116.368 | 1.00 | 194.53 | N |
| ATOM | 2743 | N | LYS | A | 425 | −31.097 | −64.823 | 110.173 | 1.00 | 194.20 | N |
| ATOM | 2744 | CA | LYS | A | 425 | −32.088 | −64.866 | 109.092 | 1.00 | 194.92 | C |
| ATOM | 2745 | C | LYS | A | 425 | −32.901 | −66.166 | 109.043 | 1.00 | 195.27 | C |
| ATOM | 2746 | O | LYS | A | 425 | −32.624 | −67.129 | 109.772 | 1.00 | 195.36 | O |
| ATOM | 2747 | CB | LYS | A | 425 | −31.430 | −64.619 | 107.720 | 1.00 | 194.96 | C |
| ATOM | 2748 | CG | LYS | A | 425 | −30.663 | −63.301 | 107.606 | 1.00 | 195.28 | C |
| ATOM | 2749 | CD | LYS | A | 425 | −30.970 | −62.519 | 106.321 | 1.00 | 194.71 | C |
| ATOM | 2750 | CE | LYS | A | 425 | −32.339 | −61.832 | 106.352 | 1.00 | 194.47 | C |
| ATOM | 2751 | NZ | LYS | A | 425 | −32.778 | −61.257 | 107.678 | 1.00 | 194.18 | N |
| ATOM | 2752 | N | VAL | A | 426 | −33.912 | −66.171 | 108.175 | 1.00 | 195.72 | N |
| ATOM | 2753 | CA | VAL | A | 426 | −34.733 | −67.354 | 107.918 | 1.00 | 196.15 | C |
| ATOM | 2754 | C | VAL | A | 426 | −34.844 | −67.619 | 106.408 | 1.00 | 196.45 | C |
| ATOM | 2755 | O | VAL | A | 426 | −35.724 | −67.077 | 105.726 | 1.00 | 196.74 | O |
| ATOM | 2756 | CB | VAL | A | 426 | −36.145 | −67.276 | 108.608 | 1.00 | 196.12 | C |
| ATOM | 2757 | CG1 | VAL | A | 426 | −36.935 | −66.039 | 108.164 | 1.00 | 195.86 | C |
| ATOM | 2758 | CG2 | VAL | A | 426 | −36.946 | −68.555 | 108.364 | 1.00 | 196.15 | C |
| ATOM | 2759 | N | ARG | A | 427 | −33.941 | −68.447 | 105.889 | 1.00 | 196.50 | N |
| ATOM | 2760 | CA | ARG | A | 427 | −33.982 | −68.801 | 104.475 | 1.00 | 196.47 | C |
| ATOM | 2761 | C | ARG | A | 427 | −34.549 | −70.192 | 104.220 | 1.00 | 196.15 | C |
| ATOM | 2762 | O | ARG | A | 427 | −34.309 | −71.131 | 104.987 | 1.00 | 196.09 | O |
| ATOM | 2763 | CB | ARG | A | 427 | −32.614 | −68.601 | 103.793 | 1.00 | 196.69 | C |
| ATOM | 2764 | CG | ARG | A | 427 | −31.383 | −68.968 | 104.619 | 1.00 | 197.55 | C |
| ATOM | 2765 | CD | ARG | A | 427 | −30.944 | −70.411 | 104.404 | 1.00 | 199.66 | C |
| ATOM | 2766 | NE | ARG | A | 427 | −30.290 | −70.620 | 103.110 | 1.00 | 201.25 | N |
| ATOM | 2767 | CZ | ARG | A | 427 | −29.574 | −71.700 | 102.797 | 1.00 | 202.56 | C |
| ATOM | 2768 | NH1 | ARG | A | 427 | −29.402 | −72.678 | 103.689 | 1.00 | 203.27 | N |
| ATOM | 2769 | NH2 | ARG | A | 427 | −29.020 | −71.805 | 101.592 | 1.00 | 202.78 | N |
| ATOM | 2770 | N | PHE | A | 428 | −35.326 | −70.295 | 103.146 | 1.00 | 195.78 | N |
| ATOM | 2771 | CA | PHE | A | 428 | −35.771 | −71.570 | 102.616 | 1.00 | 195.53 | C |
| ATOM | 2772 | C | PHE | A | 428 | −34.605 | −72.539 | 102.524 | 1.00 | 195.97 | C |
| ATOM | 2773 | O | PHE | A | 428 | −33.499 | −72.166 | 102.141 | 1.00 | 195.80 | O |
| ATOM | 2774 | CB | PHE | A | 428 | −36.342 | −71.372 | 101.218 | 1.00 | 195.04 | C |
| ATOM | 2775 | CG | PHE | A | 428 | −37.724 | −70.811 | 101.194 | 1.00 | 193.94 | C |
| ATOM | 2776 | CD1 | PHE | A | 428 | −38.021 | −69.608 | 101.809 | 1.00 | 193.15 | C |
| ATOM | 2777 | CD2 | PHE | A | 428 | −38.731 | −71.478 | 100.525 | 1.00 | 193.19 | C |
| ATOM | 2778 | CE1 | PHE | A | 428 | −39.310 | −69.097 | 101.770 | 1.00 | 193.00 | C |
| ATOM | 2779 | CE2 | PHE | A | 428 | −40.016 | −70.964 | 100.481 | 1.00 | 192.99 | C |
| ATOM | 2780 | CZ | PHE | A | 428 | −40.306 | −69.772 | 101.105 | 1.00 | 193.22 | C |
| ATOM | 2781 | N | MET | A | 429 | −34.854 | −73.786 | 102.885 | 1.00 | 196.72 | N |
| ATOM | 2782 | CA | MET | A | 429 | −33.866 | −74.828 | 102.702 | 1.00 | 197.64 | C |
| ATOM | 2783 | C | MET | A | 429 | −34.558 | −76.180 | 102.558 | 1.00 | 197.86 | C |
| ATOM | 2784 | O | MET | A | 429 | −35.448 | −76.512 | 103.338 | 1.00 | 197.80 | O |
| ATOM | 2785 | CB | MET | A | 429 | −32.839 | −74.809 | 103.840 | 1.00 | 198.05 | C |
| ATOM | 2786 | CG | MET | A | 429 | −31.497 | −75.457 | 103.487 | 1.00 | 199.59 | C |
| ATOM | 2787 | SD | MET | A | 429 | −31.178 | −75.679 | 101.701 | 1.00 | 203.00 | S |
| ATOM | 2788 | CE | MET | A | 429 | −29.511 | −76.346 | 101.703 | 1.00 | 200.80 | C |
| ATOM | 2789 | N | ALA | A | 430 | −34.149 | −76.941 | 101.542 | 1.00 | 198.31 | N |
| ATOM | 2790 | CA | ALA | A | 430 | −34.791 | −78.207 | 101.190 | 1.00 | 198.82 | C |
| ATOM | 2791 | C | ALA | A | 430 | −34.140 | −79.401 | 101.862 | 1.00 | 199.33 | C |
| ATOM | 2792 | O | ALA | A | 430 | −32.932 | −79.401 | 102.100 | 1.00 | 199.31 | O |
| ATOM | 2793 | CB | ALA | A | 430 | −34.795 | −78.397 | 99.696 | 1.00 | 198.83 | C |
| ATOM | 2794 | N | TYR | A | 431 | −34.959 | −80.410 | 102.166 | 1.00 | 200.09 | N |
| ATOM | 2795 | CA | TYR | A | 431 | −34.510 | −81.660 | 102.802 | 1.00 | 200.83 | C |
| ATOM | 2796 | C | TYR | A | 431 | −34.685 | −82.859 | 101.862 | 1.00 | 201.06 | C |
| ATOM | 2797 | O | TYR | A | 431 | −35.238 | −82.728 | 100.757 | 1.00 | 201.16 | O |
| ATOM | 2798 | CB | TYR | A | 431 | −35.211 | −81.890 | 104.157 | 1.00 | 201.01 | C |
| ATOM | 2799 | CG | TYR | A | 431 | −34.601 | −81.085 | 105.284 | 1.00 | 201.41 | C |
| ATOM | 2800 | CD1 | TYR | A | 431 | −33.622 | −81.638 | 106.103 | 1.00 | 202.25 | C |
| ATOM | 2801 | CD2 | TYR | A | 431 | −34.983 | −79.756 | 105.515 | 1.00 | 201.62 | C |
| ATOM | 2802 | CE1 | TYR | A | 431 | −33.036 | −80.891 | 107.142 | 1.00 | 202.88 | C |
| ATOM | 2803 | CE2 | TYR | A | 431 | −34.405 | −78.995 | 106.548 | 1.00 | 201.88 | C |
| ATOM | 2804 | CZ | TYR | A | 431 | −33.431 | −79.570 | 107.360 | 1.00 | 202.27 | C |
| ATOM | 2805 | OH | TYR | A | 431 | −32.849 | −78.846 | 108.388 | 1.00 | 201.87 | O |
| ATOM | 2806 | N | THR | A | 432 | −34.208 | −84.020 | 102.302 | 1.00 | 201.14 | N |
| ATOM | 2807 | CA | THR | A | 432 | −34.150 | −85.184 | 101.442 | 1.00 | 201.26 | C |
| ATOM | 2808 | C | THR | A | 432 | −35.502 | −85.823 | 101.272 | 1.00 | 201.57 | C |
| ATOM | 2809 | O | THR | A | 432 | −35.881 | −86.157 | 100.159 | 1.00 | 201.71 | O |
| ATOM | 2810 | CB | THR | A | 432 | −33.211 | −86.221 | 101.999 | 1.00 | 201.16 | C |
| ATOM | 2811 | OG1 | THR | A | 432 | −32.166 | −85.557 | 102.710 | 1.00 | 201.25 | O |
| ATOM | 2812 | CG2 | THR | A | 432 | −32.627 | −87.051 | 100.873 | 1.00 | 201.15 | C |
| ATOM | 2813 | N | ASP | A | 433 | −36.224 | −85.996 | 102.374 | 1.00 | 201.97 | N |
| ATOM | 2814 | CA | ASP | A | 433 | −37.490 | −86.720 | 102.360 | 1.00 | 202.68 | C |
| ATOM | 2815 | C | ASP | A | 433 | −38.356 | −86.326 | 103.536 | 1.00 | 203.14 | C |
| ATOM | 2816 | O | ASP | A | 433 | −37.918 | −85.570 | 104.403 | 1.00 | 203.11 | O |
| ATOM | 2817 | CB | ASP | A | 433 | −37.241 | −88.225 | 102.386 | 1.00 | 202.81 | C |
| ATOM | 2818 | CG | ASP | A | 433 | −36.132 | −88.617 | 103.349 | 1.00 | 203.46 | C |
| ATOM | 2819 | OD1 | ASP | A | 433 | −35.964 | −89.831 | 103.617 | 1.00 | 203.91 | O |
| ATOM | 2820 | OD2 | ASP | A | 433 | −35.423 | −87.708 | 103.837 | 1.00 | 204.12 | O |
| ATOM | 2821 | N | GLU | A | 434 | −39.581 | −86.855 | 103.558 | 1.00 | 203.91 | N |
| ATOM | 2822 | CA | GLU | A | 434 | −40.597 | −86.526 | 104.570 | 1.00 | 204.76 | C |
| ATOM | 2823 | C | GLU | A | 434 | −40.059 | −86.469 | 106.003 | 1.00 | 204.89 | C |
| ATOM | 2824 | O | GLU | A | 434 | −40.398 | −85.566 | 106.773 | 1.00 | 204.91 | O |
| ATOM | 2825 | CB | GLU | A | 434 | −41.775 | −87.502 | 104.470 | 1.00 | 205.09 | C |
| ATOM | 2826 | CG | GLU | A | 434 | −42.914 | −87.023 | 103.550 | 1.00 | 206.41 | C |
| ATOM | 2827 | CD | GLU | A | 434 | −44.088 | −86.365 | 104.303 | 1.00 | 208.04 | C |
| ATOM | 2828 | OE1 | GLU | A | 434 | −45.147 | −86.151 | 103.668 | 1.00 | 208.33 | O |
| ATOM | 2829 | OE2 | GLU | A | 434 | −43.967 | −86.071 | 105.520 | 1.00 | 208.79 | O |
| ATOM | 2830 | N | THR | A | 435 | −39.233 | −87.447 | 106.354 | 1.00 | 205.16 | N |
| ATOM | 2831 | CA | THR | A | 435 | −38.397 | −87.356 | 107.542 | 1.00 | 205.42 | C |
| ATOM | 2832 | C | THR | A | 435 | −37.460 | −86.165 | 107.355 | 1.00 | 205.55 | C |
| ATOM | 2833 | O | THR | A | 435 | −36.369 | −86.305 | 106.805 | 1.00 | 205.66 | O |
| ATOM | 2834 | CB | THR | A | 435 | −37.603 | −88.682 | 107.804 | 1.00 | 205.49 | C |
| ATOM | 2835 | OG1 | THR | A | 435 | −36.249 | −88.384 | 108.172 | 1.00 | 205.55 | O |
| ATOM | 2836 | CG2 | THR | A | 435 | −37.590 | −89.588 | 106.569 | 1.00 | 205.49 | C |
| ATOM | 2837 | N | PHE | A | 436 | −37.895 | −84.988 | 107.787 | 1.00 | 205.72 | N |
| ATOM | 2838 | CA | PHE | A | 436 | −37.147 | −83.774 | 107.500 | 1.00 | 206.15 | C |
| ATOM | 2839 | C | PHE | A | 436 | −35.840 | −83.691 | 108.273 | 1.00 | 206.58 | C |
| ATOM | 2840 | O | PHE | A | 436 | −35.594 | −82.730 | 108.995 | 1.00 | 206.56 | O |
| ATOM | 2841 | CB | PHE | A | 436 | −38.010 | −82.553 | 107.769 | 1.00 | 206.06 | C |
| ATOM | 2842 | CG | PHE | A | 436 | −38.781 | −82.083 | 106.570 | 1.00 | 206.08 | C |
| ATOM | 2843 | CD1 | PHE | A | 436 | −39.981 | −82.694 | 106.210 | 1.00 | 206.07 | C |
| ATOM | 2844 | CD2 | PHE | A | 436 | −38.318 | −81.018 | 105.806 | 1.00 | 205.63 | C |
| ATOM | 2845 | CE1 | PHE | A | 436 | −40.705 | −82.255 | 105.101 | 1.00 | 205.59 | C |
| ATOM | 2846 | CE2 | PHE | A | 436 | −39.034 | −80.569 | 104.697 | 1.00 | 205.53 | C |
| ATOM | 2847 | CZ | PHE | A | 436 | −40.230 | −81.189 | 104.343 | 1.00 | 205.52 | C |
| ATOM | 2848 | N | LYS | A | 437 | −34.993 | −84.700 | 108.101 | 1.00 | 207.26 | N |
| ATOM | 2849 | CA | LYS | A | 437 | −33.767 | −84.797 | 108.874 | 1.00 | 208.06 | C |
| ATOM | 2850 | C | LYS | A | 437 | −32.505 | −84.529 | 108.070 | 1.00 | 208.51 | C |
| ATOM | 2851 | O | LYS | A | 437 | −31.723 | −83.650 | 108.443 | 1.00 | 208.55 | O |
| ATOM | 2852 | CB | LYS | A | 437 | −33.668 | −86.143 | 109.604 | 1.00 | 208.09 | C |
| ATOM | 2853 | CG | LYS | A | 437 | −33.897 | −86.046 | 111.125 | 1.00 | 208.96 | C |
| ATOM | 2854 | CD | LYS | A | 437 | −35.121 | −86.829 | 111.648 | 1.00 | 210.38 | C |
| ATOM | 2855 | CE | LYS | A | 437 | −36.449 | −86.383 | 111.029 | 1.00 | 211.87 | C |
| ATOM | 2856 | NZ | LYS | A | 437 | −36.767 | −84.926 | 111.187 | 1.00 | 212.96 | N |
| ATOM | 2857 | N | THR | A | 438 | −32.306 | −85.270 | 106.977 | 1.00 | 209.24 | N |
| ATOM | 2858 | CA | THR | A | 438 | −31.038 | −85.189 | 106.228 | 1.00 | 209.92 | C |
| ATOM | 2859 | C | THR | A | 438 | −30.837 | −83.831 | 105.519 | 1.00 | 210.44 | C |
| ATOM | 2860 | O | THR | A | 438 | −31.498 | −83.501 | 104.520 | 1.00 | 210.25 | O |
| ATOM | 2861 | CB | THR | A | 438 | −30.770 | −86.406 | 105.264 | 1.00 | 209.90 | C |
| ATOM | 2862 | OG1 | THR | A | 438 | −31.574 | −87.534 | 105.629 | 1.00 | 209.91 | O |
| ATOM | 2863 | CG2 | THR | A | 438 | −29.293 | −86.812 | 105.299 | 1.00 | 209.52 | C |
| ATOM | 2864 | N | ARG | A | 439 | −29.921 | −83.061 | 106.107 | 1.00 | 211.20 | N |
| ATOM | 2865 | CA | ARG | A | 439 | −29.423 | −81.784 | 105.603 | 1.00 | 211.87 | C |
| ATOM | 2866 | C | ARG | A | 439 | −29.113 | −81.884 | 104.104 | 1.00 | 212.41 | C |
| ATOM | 2867 | O | ARG | A | 439 | −28.328 | −82.738 | 103.688 | 1.00 | 212.39 | O |
| ATOM | 2868 | CB | ARG | A | 439 | −28.156 | −81.421 | 106.416 | 1.00 | 211.78 | C |
| ATOM | 2869 | CG | ARG | A | 439 | −27.541 | −80.032 | 106.191 | 1.00 | 211.29 | C |
| ATOM | 2870 | CD | ARG | A | 439 | −28.078 | −78.965 | 107.157 | 1.00 | 209.41 | C |
| ATOM | 2871 | NE | ARG | A | 439 | −29.480 | −78.642 | 106.901 | 1.00 | 207.33 | N |
| ATOM | 2872 | CZ | ARG | A | 439 | −29.918 | −77.905 | 105.885 | 1.00 | 205.87 | C |
| ATOM | 2873 | NH1 | ARG | A | 439 | −29.075 | −77.387 | 105.001 | 1.00 | 205.00 | N |
| ATOM | 2874 | NH2 | ARG | A | 439 | −31.213 | −77.687 | 105.756 | 1.00 | 205.38 | N |
| ATOM | 2875 | N | GLU | A | 440 | −29.738 | −81.037 | 103.291 | 1.00 | 213.15 | N |
| ATOM | 2876 | CA | GLU | A | 440 | −29.417 | −81.028 | 101.863 | 1.00 | 214.09 | C |
| ATOM | 2877 | C | GLU | A | 440 | −28.146 | −80.250 | 101.576 | 1.00 | 214.49 | C |
| ATOM | 2878 | O | GLU | A | 440 | −28.098 | −79.045 | 101.826 | 1.00 | 214.49 | O |
| ATOM | 2879 | CB | GLU | A | 440 | −30.567 | −80.474 | 101.028 | 1.00 | 214.20 | C |
| ATOM | 2880 | CG | GLU | A | 440 | −31.588 | −81.531 | 100.605 | 1.00 | 215.04 | C |
| ATOM | 2881 | CD | GLU | A | 440 | −31.118 | −82.441 | 99.467 | 1.00 | 215.75 | C |
| ATOM | 2882 | OE1 | GLU | A | 440 | −31.848 | −83.420 | 99.169 | 1.00 | 215.63 | O |
| ATOM | 2883 | OE2 | GLU | A | 440 | −30.039 | −82.182 | 98.875 | 1.00 | 216.01 | O |
| ATOM | 2884 | N | ALA | A | 441 | −27.128 | −80.940 | 101.050 | 1.00 | 215.05 | N |
| ATOM | 2885 | CA | ALA | A | 441 | −25.830 | −80.323 | 100.753 | 1.00 | 215.61 | C |
| ATOM | 2886 | C | ALA | A | 441 | −26.040 | −79.002 | 100.022 | 1.00 | 216.07 | C |
| ATOM | 2887 | O | ALA | A | 441 | −26.755 | −78.938 | 99.018 | 1.00 | 216.11 | O |
| ATOM | 2888 | CB | ALA | A | 441 | −24.946 | −81.263 | 99.950 | 1.00 | 215.51 | C |
| ATOM | 2889 | N | ILE | A | 442 | −25.431 | −77.948 | 100.554 | 1.00 | 216.75 | N |
| ATOM | 2890 | CA | ILE | A | 442 | −25.762 | −76.573 | 100.156 | 1.00 | 217.41 | C |
| ATOM | 2891 | C | ILE | A | 442 | −24.989 | −76.121 | 98.900 | 1.00 | 217.62 | C |
| ATOM | 2892 | O | ILE | A | 442 | −24.048 | −76.785 | 98.453 | 1.00 | 217.57 | O |
| ATOM | 2893 | CB | ILE | A | 442 | −25.597 | −75.534 | 101.354 | 1.00 | 217.57 | C |
| ATOM | 2894 | CG1 | ILE | A | 442 | −25.864 | −76.174 | 102.730 | 1.00 | 217.95 | C |
| ATOM | 2895 | CG2 | ILE | A | 442 | −26.515 | −74.310 | 101.174 | 1.00 | 217.43 | C |
| ATOM | 2896 | CD1 | ILE | A | 442 | −24.621 | −76.737 | 103.447 | 1.00 | 218.18 | C |
| ATOM | 2897 | N | GLN | A | 443 | −25.429 | −74.996 | 98.333 | 1.00 | 217.97 | N |
| ATOM | 2898 | CA | GLN | A | 443 | −24.770 | −74.330 | 97.203 | 1.00 | 218.13 | C |
| ATOM | 2899 | C | GLN | A | 443 | −24.187 | −72.962 | 97.612 | 1.00 | 217.76 | C |
| ATOM | 2900 | O | GLN | A | 443 | −24.725 | −71.903 | 97.267 | 1.00 | 217.73 | O |
| ATOM | 2901 | CB | GLN | A | 443 | −25.750 | −74.185 | 96.027 | 1.00 | 218.42 | C |
| ATOM | 2902 | CG | GLN | A | 443 | −25.557 | −75.201 | 94.905 | 1.00 | 219.37 | C |
| ATOM | 2903 | CD | GLN | A | 443 | −24.438 | −74.801 | 93.956 | 1.00 | 220.66 | C |
| ATOM | 2904 | OE1 | GLN | A | 443 | −23.252 | −74.902 | 94.293 | 1.00 | 221.38 | O |
| ATOM | 2905 | NE2 | GLN | A | 443 | −24.812 | −74.334 | 92.763 | 1.00 | 220.86 | N |
| ATOM | 2906 | N | HIS | A | 444 | −23.084 | −73.018 | 98.353 | 1.00 | 217.26 | N |
| ATOM | 2907 | CA | HIS | A | 444 | −22.355 | −71.859 | 98.891 | 1.00 | 216.79 | C |
| ATOM | 2908 | C | HIS | A | 444 | −22.065 | −70.715 | 97.902 | 1.00 | 216.14 | C |
| ATOM | 2909 | O | HIS | A | 444 | −21.353 | −69.766 | 98.253 | 1.00 | 216.18 | O |
| ATOM | 2910 | CB | HIS | A | 444 | −21.051 | −72.376 | 99.489 | 1.00 | 216.99 | C |
| ATOM | 2911 | CG | HIS | A | 444 | −20.679 | −73.730 | 98.973 | 1.00 | 217.80 | C |
| ATOM | 2912 | ND1 | HIS | A | 444 | −20.400 | −73.966 | 97.642 | 1.00 | 218.37 | N |
| ATOM | 2913 | CD2 | HIS | A | 444 | −20.601 | −74.931 | 99.593 | 1.00 | 218.52 | C |
| ATOM | 2914 | CE1 | HIS | A | 444 | −20.140 | −75.249 | 97.470 | 1.00 | 218.80 | C |
| ATOM | 2915 | NE2 | HIS | A | 444 | −20.251 | −75.857 | 98.639 | 1.00 | 219.07 | N |
| ATOM | 2916 | N | GLU | A | 445 | −22.594 | −70.815 | 96.678 | 1.00 | 215.13 | N |
| ATOM | 2917 | CA | GLU | A | 445 | −22.617 | −69.687 | 95.743 | 1.00 | 214.25 | C |
| ATOM | 2918 | C | GLU | A | 445 | −23.982 | −68.987 | 95.750 | 1.00 | 213.25 | C |
| ATOM | 2919 | O | GLU | A | 445 | −24.064 | −67.788 | 95.497 | 1.00 | 213.19 | O |
| ATOM | 2920 | CB | GLU | A | 445 | −22.204 | −70.097 | 94.311 | 1.00 | 214.49 | C |
| ATOM | 2921 | CG | GLU | A | 445 | −23.320 | −70.700 | 93.400 | 1.00 | 215.92 | C |
| ATOM | 2922 | CD | GLU | A | 445 | −24.406 | −69.689 | 92.927 | 1.00 | 217.29 | C |
| ATOM | 2923 | OE1 | GLU | A | 445 | −25.594 | −70.085 | 92.799 | 1.00 | 217.64 | O |
| ATOM | 2924 | OE2 | GLU | A | 445 | −24.082 | −68.503 | 92.688 | 1.00 | 217.51 | O |
| ATOM | 2925 | N | SER | A | 446 | −25.051 | −69.734 | 96.020 | 1.00 | 212.07 | N |
| ATOM | 2926 | CA | SER | A | 446 | −26.398 | −69.157 | 96.029 | 1.00 | 210.95 | C |
| ATOM | 2927 | C | SER | A | 446 | −26.710 | −68.511 | 97.381 | 1.00 | 210.04 | C |
| ATOM | 2928 | O | SER | A | 446 | −27.778 | −67.934 | 97.577 | 1.00 | 209.81 | O |
| ATOM | 2929 | CB | SER | A | 446 | −27.465 | −70.190 | 95.616 | 1.00 | 211.06 | C |
| ATOM | 2930 | OG | SER | A | 446 | −27.454 | −71.341 | 96.441 | 1.00 | 210.87 | O |
| ATOM | 2931 | N | GLY | A | 447 | −25.747 | −68.602 | 98.296 | 1.00 | 209.02 | N |
| ATOM | 2932 | CA | GLY | A | 447 | −25.829 | −67.972 | 99.608 | 1.00 | 207.74 | C |
| ATOM | 2933 | C | GLY | A | 447 | −27.185 | −68.142 | 100.254 | 1.00 | 206.81 | C |
| ATOM | 2934 | O | GLY | A | 447 | −27.590 | −69.253 | 100.600 | 1.00 | 206.85 | O |
| ATOM | 2935 | N | ILE | A | 448 | −27.894 | −67.030 | 100.388 | 1.00 | 205.75 | N |
| ATOM | 2936 | CA | ILE | A | 448 | −29.201 | −67.015 | 101.022 | 1.00 | 204.65 | C |
| ATOM | 2937 | C | ILE | A | 448 | −30.273 | −67.753 | 100.212 | 1.00 | 203.99 | C |
| ATOM | 2938 | O | ILE | A | 448 | −31.239 | −68.253 | 100.787 | 1.00 | 203.86 | O |
| ATOM | 2939 | CB | ILE | A | 448 | −29.648 | −65.564 | 101.323 | 1.00 | 204.63 | C |
| ATOM | 2940 | CG1 | ILE | A | 448 | −30.973 | −65.552 | 102.085 | 1.00 | 204.88 | C |
| ATOM | 2941 | CG2 | ILE | A | 448 | −29.729 | −64.734 | 100.044 | 1.00 | 204.13 | C |
| ATOM | 2942 | CD1 | ILE | A | 448 | −31.295 | −64.228 | 102.748 | 1.00 | 205.76 | C |
| ATOM | 2943 | N | LEU | A | 449 | −30.079 | −67.843 | 98.893 | 1.00 | 203.13 | N |
| ATOM | 2944 | CA | LEU | A | 449 | −31.123 | −68.304 | 97.952 | 1.00 | 202.31 | C |
| ATOM | 2945 | C | LEU | A | 449 | −31.687 | −69.684 | 98.212 | 1.00 | 201.65 | C |
| ATOM | 2946 | O | LEU | A | 449 | −30.956 | −70.579 | 98.634 | 1.00 | 201.95 | O |
| ATOM | 2947 | CB | LEU | A | 449 | −30.629 | −68.238 | 96.508 | 1.00 | 202.22 | C |
| ATOM | 2948 | CG | LEU | A | 449 | −31.027 | −66.943 | 95.808 | 1.00 | 202.30 | C |
| ATOM | 2949 | CD1 | LEU | A | 449 | −30.063 | −66.624 | 94.698 | 1.00 | 202.79 | C |
| ATOM | 2950 | CD2 | LEU | A | 449 | −32.453 | −67.025 | 95.283 | 1.00 | 202.34 | C |
| ATOM | 2951 | N | GLY | A | 450 | −32.985 | −69.849 | 97.943 | 1.00 | 200.63 | N |
| ATOM | 2952 | CA | GLY | A | 450 | −33.670 | −71.135 | 98.116 | 1.00 | 199.38 | C |
| ATOM | 2953 | C | GLY | A | 450 | −33.181 | −72.144 | 97.097 | 1.00 | 198.39 | C |
| ATOM | 2954 | O | GLY | A | 450 | −32.446 | −71.774 | 96.182 | 1.00 | 198.58 | O |
| ATOM | 2955 | N | PRO | A | 451 | −33.567 | −73.423 | 97.249 | 1.00 | 197.47 | N |
| ATOM | 2956 | CA | PRO | A | 451 | −33.191 | −74.467 | 96.288 | 1.00 | 197.00 | C |
| ATOM | 2957 | C | PRO | A | 451 | −33.694 | −74.134 | 94.875 | 1.00 | 196.71 | C |
| ATOM | 2958 | O | PRO | A | 451 | −34.782 | −73.554 | 94.737 | 1.00 | 196.99 | O |
| ATOM | 2959 | CB | PRO | A | 451 | −33.903 | −75.706 | 96.827 | 1.00 | 196.91 | C |
| ATOM | 2960 | CG | PRO | A | 451 | −34.966 | −75.171 | 97.728 | 1.00 | 196.92 | C |
| ATOM | 2961 | CD | PRO | A | 451 | −34.389 | −73.961 | 98.340 | 1.00 | 197.17 | C |
| ATOM | 2962 | N | LEU | A | 452 | −32.917 | −74.482 | 93.843 | 1.00 | 195.86 | N |
| ATOM | 2963 | CA | LEU | A | 452 | −33.246 | −74.081 | 92.460 | 1.00 | 195.01 | C |
| ATOM | 2964 | C | LEU | A | 452 | −34.441 | −74.836 | 91.884 | 1.00 | 194.21 | C |
| ATOM | 2965 | O | LEU | A | 452 | −34.326 | −76.000 | 91.493 | 1.00 | 194.15 | O |
| ATOM | 2966 | CB | LEU | A | 452 | −32.023 | −74.227 | 91.546 | 1.00 | 195.22 | C |
| ATOM | 2967 | CG | LEU | A | 452 | −32.037 | −73.820 | 90.055 | 1.00 | 195.66 | C |
| ATOM | 2968 | CD1 | LEU | A | 452 | −32.471 | −74.972 | 89.114 | 1.00 | 195.52 | C |
| ATOM | 2969 | CD2 | LEU | A | 452 | −32.827 | −72.522 | 89.787 | 1.00 | 195.45 | C |
| ATOM | 2970 | N | LEU | A | 453 | −35.584 | −74.168 | 91.821 | 1.00 | 193.16 | N |
| ATOM | 2971 | CA | LEU | A | 453 | −36.786 | −74.836 | 91.363 | 1.00 | 192.42 | C |
| ATOM | 2972 | C | LEU | A | 453 | −36.961 | −74.757 | 89.864 | 1.00 | 192.35 | C |
| ATOM | 2973 | O | LEU | A | 453 | −36.734 | −73.707 | 89.262 | 1.00 | 192.44 | O |
| ATOM | 2974 | CB | LEU | A | 453 | −38.009 | −74.289 | 92.068 | 1.00 | 192.18 | C |
| ATOM | 2975 | CG | LEU | A | 453 | −38.511 | −75.201 | 93.172 | 1.00 | 191.21 | C |
| ATOM | 2976 | CD1 | LEU | A | 453 | −37.555 | −75.190 | 94.332 | 1.00 | 190.72 | C |
| ATOM | 2977 | CD2 | LEU | A | 453 | −39.875 | −74.753 | 93.610 | 1.00 | 190.38 | C |
| ATOM | 2978 | N | TYR | A | 454 | −37.392 | −75.874 | 89.278 | 1.00 | 192.03 | N |
| ATOM | 2979 | CA | TYR | A | 454 | −37.432 | −76.045 | 87.826 | 1.00 | 191.57 | C |
| ATOM | 2980 | C | TYR | A | 454 | −38.604 | −76.933 | 87.422 | 1.00 | 190.81 | C |
| ATOM | 2981 | O | TYR | A | 454 | −38.932 | −77.906 | 88.101 | 1.00 | 190.56 | O |
| ATOM | 2982 | CB | TYR | A | 454 | −36.077 | −76.615 | 87.353 | 1.00 | 192.12 | C |
| ATOM | 2983 | CG | TYR | A | 454 | −35.903 | −76.976 | 85.873 | 1.00 | 192.92 | C |
| ATOM | 2984 | CD1 | TYR | A | 454 | −36.682 | −76.383 | 84.869 | 1.00 | 193.13 | C |
| ATOM | 2985 | CD2 | TYR | A | 454 | −34.900 | −77.887 | 85.477 | 1.00 | 193.54 | C |
| ATOM | 2986 | CE1 | TYR | A | 454 | −36.506 | −76.722 | 83.515 | 1.00 | 193.31 | C |
| ATOM | 2987 | CE2 | TYR | A | 454 | −34.711 | −78.226 | 84.122 | 1.00 | 193.50 | C |
| ATOM | 2988 | CZ | TYR | A | 454 | −35.521 | −77.638 | 83.148 | 1.00 | 193.22 | C |
| ATOM | 2989 | OH | TYR | A | 454 | −35.353 | −77.957 | 81.813 | 1.00 | 192.97 | O |
| ATOM | 2990 | N | GLY | A | 455 | −39.243 | −76.556 | 86.321 | 1.00 | 190.13 | N |
| ATOM | 2991 | CA | GLY | A | 455 | −40.319 | −77.332 | 85.720 | 1.00 | 189.43 | C |
| ATOM | 2992 | C | GLY | A | 455 | −40.705 | −76.816 | 84.339 | 1.00 | 188.92 | C |
| ATOM | 2993 | O | GLY | A | 455 | −41.112 | −75.652 | 84.192 | 1.00 | 188.78 | O |
| ATOM | 2994 | N | GLU | A | 456 | −40.579 | −77.688 | 83.332 | 1.00 | 188.29 | N |
| ATOM | 2995 | CA | GLU | A | 456 | −40.920 | −77.360 | 81.940 | 1.00 | 187.63 | C |
| ATOM | 2996 | C | GLU | A | 456 | −42.413 | −77.254 | 81.762 | 1.00 | 186.62 | C |
| ATOM | 2997 | O | GLU | A | 456 | −43.173 | −77.685 | 82.613 | 1.00 | 186.64 | O |
| ATOM | 2998 | CB | GLU | A | 456 | −40.431 | −78.448 | 80.992 | 1.00 | 187.96 | C |
| ATOM | 2999 | CG | GLU | A | 456 | −38.956 | −78.765 | 81.084 | 1.00 | 189.65 | C |
| ATOM | 3000 | CD | GLU | A | 456 | −38.667 | −80.227 | 80.782 | 1.00 | 191.80 | C |
| ATOM | 3001 | OE1 | GLU | A | 456 | −39.159 | −80.748 | 79.744 | 1.00 | 192.48 | O |
| ATOM | 3002 | OE2 | GLU | A | 456 | −37.945 | −80.851 | 81.594 | 1.00 | 192.29 | O |
| ATOM | 3003 | N | VAL | A | 457 | −42.844 | −76.696 | 80.646 | 1.00 | 185.52 | N |
| ATOM | 3004 | CA | VAL | A | 457 | −44.251 | −76.682 | 80.354 | 1.00 | 184.71 | C |
| ATOM | 3005 | C | VAL | A | 457 | −44.725 | −78.122 | 80.338 | 1.00 | 184.65 | C |
| ATOM | 3006 | O | VAL | A | 457 | −44.052 | −78.991 | 79.782 | 1.00 | 184.46 | O |
| ATOM | 3007 | CB | VAL | A | 457 | −44.517 | −76.071 | 79.007 | 1.00 | 184.54 | C |
| ATOM | 3008 | CG1 | VAL | A | 457 | −45.984 | −76.005 | 78.770 | 1.00 | 184.47 | C |
| ATOM | 3009 | CG2 | VAL | A | 457 | −43.930 | −74.702 | 78.942 | 1.00 | 184.25 | C |
| ATOM | 3010 | N | GLY | A | 458 | −45.867 | −78.367 | 80.981 | 1.00 | 184.76 | N |
| ATOM | 3011 | CA | GLY | A | 458 | −46.509 | −79.694 | 81.010 | 1.00 | 184.93 | C |
| ATOM | 3012 | C | GLY | A | 458 | −46.314 | −80.507 | 82.285 | 1.00 | 184.96 | C |
| ATOM | 3013 | O | GLY | A | 458 | −47.008 | −81.511 | 82.525 | 1.00 | 184.84 | O |
| ATOM | 3014 | N | ASP | A | 459 | −45.360 | −80.062 | 83.099 | 1.00 | 185.05 | N |
| ATOM | 3015 | CA | ASP | A | 459 | −44.960 | −80.770 | 84.307 | 1.00 | 184.91 | C |
| ATOM | 3016 | C | ASP | A | 459 | −45.741 | −80.293 | 85.512 | 1.00 | 184.63 | C |
| ATOM | 3017 | O | ASP | A | 459 | −46.479 | −79.305 | 85.468 | 1.00 | 184.45 | O |
| ATOM | 3018 | CB | ASP | A | 459 | −43.456 | −80.601 | 84.586 | 1.00 | 185.07 | C |
| ATOM | 3019 | CG | ASP | A | 459 | −42.575 | −81.094 | 83.443 | 1.00 | 185.35 | C |
| ATOM | 3020 | OD1 | ASP | A | 459 | −41.427 | −80.591 | 83.330 | 1.00 | 184.94 | O |
| ATOM | 3021 | OD2 | ASP | A | 459 | −43.026 | −81.975 | 82.667 | 1.00 | 185.51 | O |
| ATOM | 3022 | N | THR | A | 460 | −45.530 | −81.015 | 86.597 | 1.00 | 184.39 | N |
| ATOM | 3023 | CA | THR | A | 460 | −46.264 | −80.834 | 87.818 | 1.00 | 184.18 | C |
| ATOM | 3024 | C | THR | A | 460 | −45.233 | −80.856 | 88.954 | 1.00 | 183.78 | C |
| ATOM | 3025 | O | THR | A | 460 | −44.441 | −81.797 | 89.051 | 1.00 | 183.80 | O |
| ATOM | 3026 | CB | THR | A | 460 | −47.341 | −81.955 | 87.952 | 1.00 | 184.38 | C |
| ATOM | 3027 | OG1 | THR | A | 460 | −46.975 | −83.086 | 87.140 | 1.00 | 184.81 | O |
| ATOM | 3028 | CG2 | THR | A | 460 | −48.685 | −81.467 | 87.461 | 1.00 | 184.22 | C |
| ATOM | 3029 | N | LEU | A | 461 | −45.208 | −79.801 | 89.771 | 1.00 | 183.16 | N |
| ATOM | 3030 | CA | LEU | A | 461 | −44.312 | −79.721 | 90.924 | 1.00 | 182.65 | C |
| ATOM | 3031 | C | LEU | A | 461 | −45.049 | −80.089 | 92.200 | 1.00 | 182.75 | C |
| ATOM | 3032 | O | LEU | A | 461 | −45.928 | −79.346 | 92.628 | 1.00 | 182.96 | O |
| ATOM | 3033 | CB | LEU | A | 461 | −43.738 | −78.317 | 91.058 | 1.00 | 182.20 | C |
| ATOM | 3034 | CG | LEU | A | 461 | −42.369 | −78.099 | 90.434 | 1.00 | 182.01 | C |
| ATOM | 3035 | CD1 | LEU | A | 461 | −42.309 | −78.604 | 89.019 | 1.00 | 182.07 | C |
| ATOM | 3036 | CD2 | LEU | A | 461 | −42.031 | −76.636 | 90.457 | 1.00 | 182.20 | C |
| ATOM | 3037 | N | LEU | A | 462 | −44.714 | −81.246 | 92.786 | 1.00 | 182.73 | N |
| ATOM | 3038 | CA | LEU | A | 462 | −45.225 | −81.653 | 94.108 | 1.00 | 182.41 | C |
| ATOM | 3039 | C | LEU | A | 462 | −44.256 | −81.115 | 95.134 | 1.00 | 182.34 | C |
| ATOM | 3040 | O | LEU | A | 462 | −43.216 | −81.734 | 95.388 | 1.00 | 182.50 | O |
| ATOM | 3041 | CB | LEU | A | 462 | −45.299 | −83.172 | 94.277 | 1.00 | 182.21 | C |
| ATOM | 3042 | CG | LEU | A | 462 | −45.759 | −84.143 | 93.191 | 1.00 | 182.47 | C |
| ATOM | 3043 | CD1 | LEU | A | 462 | −45.836 | −85.512 | 93.804 | 1.00 | 182.99 | C |
| ATOM | 3044 | CD2 | LEU | A | 462 | −47.104 | −83.791 | 92.579 | 1.00 | 183.42 | C |
| ATOM | 3045 | N | ILE | A | 463 | −44.600 | −79.967 | 95.715 | 1.00 | 182.13 | N |
| ATOM | 3046 | CA | ILE | A | 463 | −43.713 | −79.253 | 96.627 | 1.00 | 181.74 | C |
| ATOM | 3047 | C | ILE | A | 463 | −44.202 | −79.362 | 98.084 | 1.00 | 181.56 | C |
| ATOM | 3048 | O | ILE | A | 463 | −45.165 | −78.706 | 98.504 | 1.00 | 181.22 | O |
| ATOM | 3049 | CB | ILE | A | 463 | −43.435 | −77.801 | 96.115 | 1.00 | 181.75 | C |
| ATOM | 3050 | CG1 | ILE | A | 463 | −42.134 | −77.245 | 96.701 | 1.00 | 182.03 | C |
| ATOM | 3051 | CG2 | ILE | A | 463 | −44.647 | −76.882 | 96.293 | 1.00 | 181.60 | C |
| ATOM | 3052 | CD1 | ILE | A | 463 | −42.304 | −76.301 | 97.877 | 1.00 | 182.81 | C |
| ATOM | 3053 | N | ILE | A | 464 | −43.527 | −80.246 | 98.820 | 1.00 | 181.50 | N |
| ATOM | 3054 | CA | ILE | A | 464 | −43.904 | −80.620 | 100.172 | 1.00 | 181.55 | C |
| ATOM | 3055 | C | ILE | A | 464 | −43.080 | −79.851 | 101.190 | 1.00 | 181.92 | C |
| ATOM | 3056 | O | ILE | A | 464 | −41.871 | −80.071 | 101.340 | 1.00 | 181.81 | O |
| ATOM | 3057 | CB | ILE | A | 464 | −43.736 | −82.113 | 100.418 | 1.00 | 181.32 | C |
| ATOM | 3058 | CG1 | ILE | A | 464 | −44.182 | −82.908 | 99.201 | 1.00 | 181.18 | C |
| ATOM | 3059 | CG2 | ILE | A | 464 | −44.567 | −82.516 | 101.604 | 1.00 | 181.52 | C |
| ATOM | 3060 | CD1 | ILE | A | 464 | −44.293 | −84.391 | 99.451 | 1.00 | 181.67 | C |
| ATOM | 3061 | N | PHE | A | 465 | −43.784 | −78.992 | 101.923 | 1.00 | 182.40 | N |
| ATOM | 3062 | CA | PHE | A | 465 | −43.199 | −77.866 | 102.639 | 1.00 | 182.70 | C |
| ATOM | 3063 | C | PHE | A | 465 | −43.413 | −77.827 | 104.164 | 1.00 | 183.32 | C |
| ATOM | 3064 | O | PHE | A | 465 | −44.548 | −77.697 | 104.645 | 1.00 | 183.32 | O |
| ATOM | 3065 | CB | PHE | A | 465 | −43.749 | −76.597 | 102.017 | 1.00 | 182.29 | C |
| ATOM | 3066 | CG | PHE | A | 465 | −43.504 | −75.389 | 102.819 | 1.00 | 181.75 | C |
| ATOM | 3067 | CD1 | PHE | A | 465 | −42.217 | −74.925 | 103.018 | 1.00 | 181.74 | C |
| ATOM | 3068 | CD2 | PHE | A | 465 | −44.556 | −74.701 | 103.375 | 1.00 | 181.85 | C |
| ATOM | 3069 | CE1 | PHE | A | 465 | −41.982 | −73.785 | 103.765 | 1.00 | 181.98 | C |
| ATOM | 3070 | CE2 | PHE | A | 465 | −44.332 | −73.559 | 104.124 | 1.00 | 182.33 | C |
| ATOM | 3071 | CZ | PHE | A | 465 | −43.043 | −73.096 | 104.314 | 1.00 | 182.03 | C |
| ATOM | 3072 | N | LYS | A | 466 | −42.302 | −77.911 | 104.905 | 1.00 | 184.08 | N |
| ATOM | 3073 | CA | LYS | A | 466 | −42.291 | −77.833 | 106.381 | 1.00 | 184.44 | C |
| ATOM | 3074 | C | LYS | A | 466 | −41.833 | −76.466 | 106.873 | 1.00 | 184.52 | C |
| ATOM | 3075 | O | LYS | A | 466 | −40.885 | −75.877 | 106.344 | 1.00 | 184.51 | O |
| ATOM | 3076 | CB | LYS | A | 466 | −41.373 | −78.911 | 106.998 | 1.00 | 184.45 | C |
| ATOM | 3077 | CG | LYS | A | 466 | −41.469 | −79.074 | 108.542 | 1.00 | 184.35 | C |
| ATOM | 3078 | CD | LYS | A | 466 | −40.785 | −80.371 | 109.019 | 1.00 | 184.77 | C |
| ATOM | 3079 | CE | LYS | A | 466 | −41.497 | −81.044 | 110.207 | 1.00 | 185.18 | C |
| ATOM | 3080 | NZ | LYS | A | 466 | −40.869 | −80.743 | 111.525 | 1.00 | 185.47 | N |
| ATOM | 3081 | N | ASN | A | 467 | −42.515 | −75.970 | 107.893 | 1.00 | 184.65 | N |
| ATOM | 3082 | CA | ASN | A | 467 | −42.000 | −74.852 | 108.644 | 1.00 | 184.85 | C |
| ATOM | 3083 | C | ASN | A | 467 | −41.197 | −75.408 | 109.805 | 1.00 | 185.09 | C |
| ATOM | 3084 | O | ASN | A | 467 | −41.634 | −76.321 | 110.506 | 1.00 | 185.03 | O |
| ATOM | 3085 | CB | ASN | A | 467 | −43.132 | −73.943 | 109.130 | 1.00 | 184.82 | C |
| ATOM | 3086 | CG | ASN | A | 467 | −42.637 | −72.581 | 109.602 | 1.00 | 184.58 | C |
| ATOM | 3087 | OD1 | ASN | A | 467 | −41.439 | −72.377 | 109.816 | 1.00 | 184.02 | O |
| ATOM | 3088 | ND2 | ASN | A | 467 | −43.566 | −71.642 | 109.768 | 1.00 | 184.19 | N |
| ATOM | 3089 | N | GLN | A | 468 | −39.999 | −74.878 | 109.975 | 1.00 | 185.55 | N |
| ATOM | 3090 | CA | GLN | A | 468 | −39.171 | −75.236 | 111.102 | 1.00 | 186.25 | C |
| ATOM | 3091 | C | GLN | A | 468 | −38.598 | −73.955 | 111.672 | 1.00 | 186.84 | C |
| ATOM | 3092 | O | GLN | A | 468 | −37.511 | −73.922 | 112.250 | 1.00 | 187.04 | O |
| ATOM | 3093 | CB | GLN | A | 468 | −38.102 | −76.246 | 110.690 | 1.00 | 186.20 | C |
| ATOM | 3094 | CG | GLN | A | 468 | −38.642 | −77.675 | 110.614 | 1.00 | 186.22 | C |
| ATOM | 3095 | CD | GLN | A | 468 | −37.578 | −78.706 | 110.296 | 1.00 | 186.27 | C |
| ATOM | 3096 | OE1 | GLN | A | 468 | −36.570 | −78.401 | 109.646 | 1.00 | 186.53 | O |
| ATOM | 3097 | NE2 | GLN | A | 468 | −37.798 | −79.941 | 110.748 | 1.00 | 185.91 | N |
| ATOM | 3098 | N | ALA | A | 469 | −39.368 | −72.892 | 111.487 | 1.00 | 187.57 | N |
| ATOM | 3099 | CA | ALA | A | 469 | −39.114 | −71.611 | 112.104 | 1.00 | 188.48 | C |
| ATOM | 3100 | C | ALA | A | 469 | −40.361 | −71.229 | 112.889 | 1.00 | 189.23 | C |
| ATOM | 3101 | O | ALA | A | 469 | −41.480 | −71.421 | 112.410 | 1.00 | 189.40 | O |
| ATOM | 3102 | CB | ALA | A | 469 | −38.827 | −70.590 | 111.048 | 1.00 | 188.43 | C |
| ATOM | 3103 | N | SER | A | 470 | −40.166 | −70.691 | 114.091 | 1.00 | 190.15 | N |
| ATOM | 3104 | CA | SER | A | 470 | −41.270 | −70.329 | 114.990 | 1.00 | 191.05 | C |
| ATOM | 3105 | C | SER | A | 470 | −42.360 | −69.465 | 114.325 | 1.00 | 191.57 | C |
| ATOM | 3106 | O | SER | A | 470 | −43.480 | −69.932 | 114.082 | 1.00 | 191.75 | O |
| ATOM | 3107 | CB | SER | A | 470 | −40.718 | −69.612 | 116.226 | 1.00 | 191.09 | C |
| ATOM | 3108 | OG | SER | A | 470 | −39.972 | −68.462 | 115.847 | 1.00 | 191.36 | O |
| ATOM | 3109 | N | ARG | A | 471 | −42.009 | −68.214 | 114.034 | 1.00 | 192.11 | N |
| ATOM | 3110 | CA | ARG | A | 471 | −42.909 | −67.221 | 113.445 | 1.00 | 192.53 | C |
| ATOM | 3111 | C | ARG | A | 471 | −43.368 | −67.641 | 112.019 | 1.00 | 192.72 | C |
| ATOM | 3112 | O | ARG | A | 471 | −42.604 | −67.505 | 111.064 | 1.00 | 192.91 | O |
| ATOM | 3113 | CB | ARG | A | 471 | −42.196 | −65.855 | 113.489 | 1.00 | 192.49 | C |
| ATOM | 3114 | CG | ARG | A | 471 | −42.884 | −64.695 | 112.824 | 1.00 | 192.79 | C |
| ATOM | 3115 | CD | ARG | A | 471 | −44.321 | −64.493 | 113.265 | 1.00 | 193.90 | C |
| ATOM | 3116 | NE | ARG | A | 471 | −44.443 | −63.634 | 114.435 | 1.00 | 194.83 | N |
| ATOM | 3117 | CZ | ARG | A | 471 | −45.352 | −62.671 | 114.564 | 1.00 | 195.05 | C |
| ATOM | 3118 | NH1 | ARG | A | 471 | −46.213 | −62.415 | 113.582 | 1.00 | 194.79 | N |
| ATOM | 3119 | NH2 | ARG | A | 471 | −45.391 | −61.948 | 115.676 | 1.00 | 195.25 | N |
| ATOM | 3120 | N | PRO | A | 472 | −44.625 | −68.139 | 111.881 | 1.00 | 192.78 | N |
| ATOM | 3121 | CA | PRO | A | 472 | −45.109 | −68.935 | 110.734 | 1.00 | 192.78 | C |
| ATOM | 3122 | C | PRO | A | 472 | −45.094 | −68.261 | 109.347 | 1.00 | 192.82 | C |
| ATOM | 3123 | O | PRO | A | 472 | −45.398 | −67.066 | 109.246 | 1.00 | 192.94 | O |
| ATOM | 3124 | CB | PRO | A | 472 | −46.553 | −69.251 | 111.134 | 1.00 | 192.72 | C |
| ATOM | 3125 | CG | PRO | A | 472 | −46.935 | −68.146 | 112.032 | 1.00 | 192.68 | C |
| ATOM | 3126 | CD | PRO | A | 472 | −45.711 | −67.922 | 112.854 | 1.00 | 192.80 | C |
| ATOM | 3127 | N | TYR | A | 473 | −44.761 | −69.044 | 108.307 | 1.00 | 192.64 | N |
| ATOM | 3128 | CA | TYR | A | 473 | −44.769 | −68.603 | 106.892 | 1.00 | 192.50 | C |
| ATOM | 3129 | C | TYR | A | 473 | −45.427 | −69.620 | 105.910 | 1.00 | 192.69 | C |
| ATOM | 3130 | O | TYR | A | 473 | −46.036 | −70.599 | 106.364 | 1.00 | 192.76 | O |
| ATOM | 3131 | CB | TYR | A | 473 | −43.348 | −68.322 | 106.398 | 1.00 | 192.24 | C |
| ATOM | 3132 | CG | TYR | A | 473 | −42.369 | −67.698 | 107.366 | 1.00 | 191.72 | C |
| ATOM | 3133 | CD1 | TYR | A | 473 | −41.331 | −68.454 | 107.899 | 1.00 | 191.61 | C |
| ATOM | 3134 | CD2 | TYR | A | 473 | −42.434 | −66.345 | 107.694 | 1.00 | 191.38 | C |
| ATOM | 3135 | CE1 | TYR | A | 473 | −40.396 | −67.892 | 108.763 | 1.00 | 191.69 | C |
| ATOM | 3136 | CE2 | TYR | A | 473 | −41.500 | −65.771 | 108.563 | 1.00 | 191.57 | C |
| ATOM | 3137 | CZ | TYR | A | 473 | −40.482 | −66.555 | 109.095 | 1.00 | 191.60 | C |
| ATOM | 3138 | OH | TYR | A | 473 | −39.552 | −66.019 | 109.961 | 1.00 | 191.26 | O |
| ATOM | 3139 | N | ASN | A | 474 | −45.303 | −69.375 | 104.584 | 1.00 | 192.70 | N |
| ATOM | 3140 | CA | ASN | A | 474 | −45.697 | −70.336 | 103.496 | 1.00 | 192.53 | C |
| ATOM | 3141 | C | ASN | A | 474 | −44.806 | −70.366 | 102.221 | 1.00 | 192.69 | C |
| ATOM | 3142 | O | ASN | A | 474 | −43.604 | −70.098 | 102.305 | 1.00 | 192.62 | O |
| ATOM | 3143 | CB | ASN | A | 474 | −47.194 | −70.233 | 103.129 | 1.00 | 192.09 | C |
| ATOM | 3144 | CG | ASN | A | 474 | −47.595 | −68.857 | 102.664 | 1.00 | 190.89 | C |
| ATOM | 3145 | OD1 | ASN | A | 474 | −46.750 | −68.018 | 102.344 | 1.00 | 188.88 | O |
| ATOM | 3146 | ND2 | ASN | A | 474 | −48.902 | −68.614 | 102.622 | 1.00 | 189.71 | N |
| ATOM | 3147 | N | ILE | A | 475 | −45.396 | −70.723 | 101.067 | 1.00 | 193.02 | N |
| ATOM | 3148 | CA | ILE | A | 475 | −44.699 | −70.778 | 99.745 | 1.00 | 193.10 | C |
| ATOM | 3149 | C | ILE | A | 475 | −45.588 | −70.541 | 98.531 | 1.00 | 193.40 | C |
| ATOM | 3150 | O | ILE | A | 475 | −46.533 | −71.289 | 98.266 | 1.00 | 193.16 | O |
| ATOM | 3151 | CB | ILE | A | 475 | −44.054 | −72.142 | 99.434 | 1.00 | 193.05 | C |
| ATOM | 3152 | CG1 | ILE | A | 475 | −44.768 | −73.257 | 100.197 | 1.00 | 193.28 | C |
| ATOM | 3153 | CG2 | ILE | A | 475 | −42.565 | −72.108 | 99.664 | 1.00 | 192.46 | C |
| ATOM | 3154 | CD1 | ILE | A | 475 | −45.013 | −74.460 | 99.370 | 1.00 | 193.99 | C |
| ATOM | 3155 | N | TYR | A | 476 | −45.246 | −69.531 | 97.753 | 1.00 | 193.87 | N |
| ATOM | 3156 | CA | TYR | A | 476 | −45.965 | −69.310 | 96.529 | 1.00 | 194.56 | C |
| ATOM | 3157 | C | TYR | A | 476 | −45.035 | −68.903 | 95.419 | 1.00 | 194.91 | C |
| ATOM | 3158 | O | TYR | A | 476 | −44.169 | −68.046 | 95.632 | 1.00 | 194.98 | O |
| ATOM | 3159 | CB | TYR | A | 476 | −47.013 | −68.239 | 96.720 | 1.00 | 194.82 | C |
| ATOM | 3160 | CG | TYR | A | 476 | −48.259 | −68.493 | 95.918 | 1.00 | 195.52 | C |
| ATOM | 3161 | CD1 | TYR | A | 476 | −49.049 | −69.609 | 96.171 | 1.00 | 196.14 | C |
| ATOM | 3162 | CD2 | TYR | A | 476 | −48.663 | −67.614 | 94.913 | 1.00 | 196.28 | C |
| ATOM | 3163 | CE1 | TYR | A | 476 | −50.211 | −69.843 | 95.451 | 1.00 | 196.18 | C |
| ATOM | 3164 | CE2 | TYR | A | 476 | −49.831 | −67.844 | 94.180 | 1.00 | 196.35 | C |
| ATOM | 3165 | CZ | TYR | A | 476 | −50.596 | −68.963 | 94.461 | 1.00 | 195.80 | C |
| ATOM | 3166 | OH | TYR | A | 476 | −51.748 | −69.216 | 93.758 | 1.00 | 195.60 | O |
| ATOM | 3167 | N | PRO | A | 477 | −45.218 | −69.510 | 94.227 | 1.00 | 195.14 | N |
| ATOM | 3168 | CA | PRO | A | 477 | −44.427 | −69.185 | 93.058 | 1.00 | 195.30 | C |
| ATOM | 3169 | C | PRO | A | 477 | −45.065 | −68.069 | 92.221 | 1.00 | 195.62 | C |
| ATOM | 3170 | O | PRO | A | 477 | −46.195 | −68.206 | 91.745 | 1.00 | 195.58 | O |
| ATOM | 3171 | CB | PRO | A | 477 | −44.404 | −70.506 | 92.292 | 1.00 | 195.09 | C |
| ATOM | 3172 | CG | PRO | A | 477 | −45.701 | −71.147 | 92.620 | 1.00 | 194.86 | C |
| ATOM | 3173 | CD | PRO | A | 477 | −46.201 | −70.567 | 93.923 | 1.00 | 195.07 | C |
| ATOM | 3174 | N | HIS | A | 478 | −44.344 | −66.959 | 92.089 | 1.00 | 196.11 | N |
| ATOM | 3175 | CA | HIS | A | 478 | −44.658 | −65.931 | 91.108 | 1.00 | 196.70 | C |
| ATOM | 3176 | C | HIS | A | 478 | −44.211 | −66.533 | 89.779 | 1.00 | 196.77 | C |
| ATOM | 3177 | O | HIS | A | 478 | −43.013 | −66.690 | 89.538 | 1.00 | 196.82 | O |
| ATOM | 3178 | CB | HIS | A | 478 | −43.896 | −64.643 | 91.449 | 1.00 | 196.93 | C |
| ATOM | 3179 | CG | HIS | A | 478 | −43.953 | −63.575 | 90.393 | 1.00 | 197.85 | C |
| ATOM | 3180 | ND1 | HIS | A | 478 | −43.185 | −63.613 | 89.245 | 1.00 | 198.43 | N |
| ATOM | 3181 | CD2 | HIS | A | 478 | −44.636 | −62.405 | 90.343 | 1.00 | 198.62 | C |
| ATOM | 3182 | CE1 | HIS | A | 478 | −43.413 | −62.530 | 88.522 | 1.00 | 198.39 | C |
| ATOM | 3183 | NE2 | HIS | A | 478 | −44.289 | −61.781 | 89.165 | 1.00 | 198.95 | N |
| ATOM | 3184 | N | GLY | A | 479 | −45.184 | −66.912 | 88.948 | 1.00 | 196.87 | N |
| ATOM | 3185 | CA | GLY | A | 479 | −44.930 | −67.646 | 87.697 | 1.00 | 196.68 | C |
| ATOM | 3186 | C | GLY | A | 479 | −46.117 | −68.489 | 87.244 | 1.00 | 196.46 | C |
| ATOM | 3187 | O | GLY | A | 479 | −46.833 | −68.117 | 86.312 | 1.00 | 196.46 | O |
| ATOM | 3188 | N | ILE | A | 480 | −46.332 | −69.623 | 87.907 | 1.00 | 196.15 | N |
| ATOM | 3189 | CA | ILE | A | 480 | −47.450 | −70.503 | 87.570 | 1.00 | 195.91 | C |
| ATOM | 3190 | C | ILE | A | 480 | −48.767 | −69.983 | 88.137 | 1.00 | 195.72 | C |
| ATOM | 3191 | O | ILE | A | 480 | −48.786 | −69.235 | 89.110 | 1.00 | 195.53 | O |
| ATOM | 3192 | CB | ILE | A | 480 | −47.220 | −71.966 | 88.028 | 1.00 | 195.98 | C |
| ATOM | 3193 | CG1 | ILE | A | 480 | −45.821 | −72.448 | 87.648 | 1.00 | 195.76 | C |
| ATOM | 3194 | CG2 | ILE | A | 480 | −48.254 | −72.899 | 87.416 | 1.00 | 195.85 | C |
| ATOM | 3195 | CD1 | ILE | A | 480 | −44.881 | −72.539 | 88.808 | 1.00 | 195.74 | C |
| ATOM | 3196 | N | THR | A | 481 | −49.860 | −70.397 | 87.510 | 1.00 | 195.67 | N |
| ATOM | 3197 | CA | THR | A | 481 | −51.184 | −69.958 | 87.889 | 1.00 | 195.79 | C |
| ATOM | 3198 | C | THR | A | 481 | −52.009 | −71.109 | 88.462 | 1.00 | 195.94 | C |
| ATOM | 3199 | O | THR | A | 481 | −52.951 | −70.883 | 89.211 | 1.00 | 196.11 | O |
| ATOM | 3200 | CB | THR | A | 481 | −51.923 | −69.322 | 86.692 | 1.00 | 195.83 | C |
| ATOM | 3201 | OG1 | THR | A | 481 | −51.974 | −70.259 | 85.607 | 1.00 | 195.85 | O |
| ATOM | 3202 | CG2 | THR | A | 481 | −51.229 | −68.015 | 86.237 | 1.00 | 195.52 | C |
| ATOM | 3203 | N | ASP | A | 482 | −51.654 | −72.342 | 88.118 | 1.00 | 196.15 | N |
| ATOM | 3204 | CA | ASP | A | 482 | −52.374 | −73.521 | 88.625 | 1.00 | 196.32 | C |
| ATOM | 3205 | C | ASP | A | 482 | −51.708 | −74.071 | 89.897 | 1.00 | 196.18 | C |
| ATOM | 3206 | O | ASP | A | 482 | −51.084 | −75.140 | 89.885 | 1.00 | 196.25 | O |
| ATOM | 3207 | CB | ASP | A | 482 | −52.474 | −74.604 | 87.528 | 1.00 | 196.55 | C |
| ATOM | 3208 | CG | ASP | A | 482 | −53.440 | −75.755 | 87.881 | 1.00 | 196.73 | C |
| ATOM | 3209 | OD1 | ASP | A | 482 | −54.040 | −76.321 | 86.938 | 1.00 | 196.70 | O |
| ATOM | 3210 | OD2 | ASP | A | 482 | −53.594 | −76.110 | 89.074 | 1.00 | 197.00 | O |
| ATOM | 3211 | N | VAL | A | 483 | −51.849 | −73.338 | 90.997 | 1.00 | 195.89 | N |
| ATOM | 3212 | CA | VAL | A | 483 | −51.258 | −73.753 | 92.274 | 1.00 | 195.66 | C |
| ATOM | 3213 | C | VAL | A | 483 | −52.322 | −74.085 | 93.326 | 1.00 | 195.52 | C |
| ATOM | 3214 | O | VAL | A | 483 | −52.986 | −73.199 | 93.885 | 1.00 | 195.62 | O |
| ATOM | 3215 | CB | VAL | A | 483 | −50.234 | −72.722 | 92.786 | 1.00 | 195.67 | C |
| ATOM | 3216 | CG1 | VAL | A | 483 | −50.636 | −71.334 | 92.355 | 1.00 | 195.48 | C |
| ATOM | 3217 | CG2 | VAL | A | 483 | −50.062 | −72.819 | 94.302 | 1.00 | 195.62 | C |
| ATOM | 3218 | N | ARG | A | 484 | −52.461 | −75.382 | 93.581 | 1.00 | 195.08 | N |
| ATOM | 3219 | CA | ARG | A | 484 | −53.549 | −75.925 | 94.379 | 1.00 | 194.56 | C |
| ATOM | 3220 | C | ARG | A | 484 | −52.965 | −76.969 | 95.331 | 1.00 | 194.29 | C |
| ATOM | 3221 | O | ARG | A | 484 | −52.032 | −77.666 | 94.961 | 1.00 | 194.40 | O |
| ATOM | 3222 | CB | ARG | A | 484 | −54.623 | −76.498 | 93.428 | 1.00 | 194.49 | C |
| ATOM | 3223 | CG | ARG | A | 484 | −55.188 | −77.865 | 93.763 | 1.00 | 194.25 | C |
| ATOM | 3224 | CD | ARG | A | 484 | −54.477 | −78.991 | 93.006 | 1.00 | 193.72 | C |
| ATOM | 3225 | NE | ARG | A | 484 | −55.292 | −79.502 | 91.906 | 1.00 | 193.27 | N |
| ATOM | 3226 | CZ | ARG | A | 484 | −55.363 | −80.782 | 91.527 | 1.00 | 192.74 | C |
| ATOM | 3227 | NH1 | ARG | A | 484 | −54.675 | −81.737 | 92.150 | 1.00 | 191.65 | N |
| ATOM | 3228 | NH2 | ARG | A | 484 | −56.150 | −81.110 | 90.515 | 1.00 | 192.70 | N |
| ATOM | 3229 | N | PRO | A | 485 | −53.493 | −77.065 | 96.566 | 1.00 | 193.98 | N |
| ATOM | 3230 | CA | PRO | A | 485 | −52.993 | −78.045 | 97.524 | 1.00 | 193.89 | C |
| ATOM | 3231 | C | PRO | A | 485 | −53.084 | −79.447 | 96.954 | 1.00 | 193.98 | C |
| ATOM | 3232 | O | PRO | A | 485 | −54.070 | −79.776 | 96.302 | 1.00 | 193.94 | O |
| ATOM | 3233 | CB | PRO | A | 485 | −53.952 | −77.902 | 98.711 | 1.00 | 193.90 | C |
| ATOM | 3234 | CG | PRO | A | 485 | −55.131 | −77.175 | 98.192 | 1.00 | 193.80 | C |
| ATOM | 3235 | CD | PRO | A | 485 | −54.596 | −76.275 | 97.132 | 1.00 | 194.02 | C |
| ATOM | 3236 | N | LEU | A | 486 | −52.068 | −80.265 | 97.216 | 1.00 | 194.21 | N |
| ATOM | 3237 | CA | LEU | A | 486 | −51.882 | −81.541 | 96.521 | 1.00 | 194.47 | C |
| ATOM | 3238 | C | LEU | A | 486 | −53.171 | −82.275 | 96.188 | 1.00 | 194.88 | C |
| ATOM | 3239 | O | LEU | A | 486 | −53.556 | −82.361 | 95.024 | 1.00 | 195.08 | O |
| ATOM | 3240 | CB | LEU | A | 486 | −50.937 | −82.468 | 97.293 | 1.00 | 194.23 | C |
| ATOM | 3241 | CG | LEU | A | 486 | −49.968 | −83.369 | 96.511 | 1.00 | 193.83 | C |
| ATOM | 3242 | CD1 | LEU | A | 486 | −50.672 | −84.366 | 95.596 | 1.00 | 193.52 | C |
| ATOM | 3243 | CD2 | LEU | A | 486 | −48.963 | −82.542 | 95.721 | 1.00 | 193.73 | C |
| ATOM | 3244 | N | TYR | A | 487 | −53.844 | −82.793 | 97.202 | 1.00 | 195.37 | N |
| ATOM | 3245 | CA | TYR | A | 487 | −54.880 | −83.769 | 96.947 | 1.00 | 196.05 | C |
| ATOM | 3246 | C | TYR | A | 487 | −56.258 | −83.221 | 96.611 | 1.00 | 196.72 | C |
| ATOM | 3247 | O | TYR | A | 487 | −56.991 | −83.833 | 95.839 | 1.00 | 196.68 | O |
| ATOM | 3248 | CB | TYR | A | 487 | −54.962 | −84.732 | 98.105 | 1.00 | 196.00 | C |
| ATOM | 3249 | CG | TYR | A | 487 | −53.809 | −85.688 | 98.147 | 1.00 | 195.88 | C |
| ATOM | 3250 | CD1 | TYR | A | 487 | −53.822 | −86.848 | 97.385 | 1.00 | 196.12 | C |
| ATOM | 3251 | CD2 | TYR | A | 487 | −52.706 | −85.442 | 98.954 | 1.00 | 195.97 | C |
| ATOM | 3252 | CE1 | TYR | A | 487 | −52.756 | −87.755 | 97.427 | 1.00 | 197.02 | C |
| ATOM | 3253 | CE2 | TYR | A | 487 | −51.630 | −86.338 | 99.011 | 1.00 | 196.47 | C |
| ATOM | 3254 | CZ | TYR | A | 487 | −51.658 | −87.497 | 98.243 | 1.00 | 196.84 | C |
| ATOM | 3255 | OH | TYR | A | 487 | −50.603 | −88.399 | 98.289 | 1.00 | 196.89 | O |
| ATOM | 3256 | N | SER | A | 488 | −56.620 | −82.082 | 97.185 | 1.00 | 197.73 | N |
| ATOM | 3257 | CA | SER | A | 488 | −57.952 | −81.532 | 96.956 | 1.00 | 198.78 | C |
| ATOM | 3258 | C | SER | A | 488 | −57.898 | −80.180 | 96.269 | 1.00 | 199.59 | C |
| ATOM | 3259 | O | SER | A | 488 | −56.835 | −79.589 | 96.107 | 1.00 | 199.54 | O |
| ATOM | 3260 | CB | SER | A | 488 | −58.732 | −81.420 | 98.276 | 1.00 | 198.78 | C |
| ATOM | 3261 | OG | SER | A | 488 | −60.063 | −80.962 | 98.066 | 1.00 | 198.64 | O |
| ATOM | 3262 | N | ARG | A | 489 | −59.066 | −79.710 | 95.857 | 1.00 | 200.78 | N |
| ATOM | 3263 | CA | ARG | A | 489 | −59.224 | −78.353 | 95.395 | 1.00 | 202.08 | C |
| ATOM | 3264 | C | ARG | A | 489 | −59.933 | −77.559 | 96.529 | 1.00 | 203.26 | C |
| ATOM | 3265 | O | ARG | A | 489 | −60.799 | −76.720 | 96.282 | 1.00 | 203.59 | O |
| ATOM | 3266 | CB | ARG | A | 489 | −59.987 | −78.354 | 94.057 | 1.00 | 201.96 | C |
| ATOM | 3267 | CG | ARG | A | 489 | −59.470 | −77.366 | 92.997 | 1.00 | 201.96 | C |
| ATOM | 3268 | CD | ARG | A | 489 | −59.736 | −77.837 | 91.548 | 1.00 | 201.88 | C |
| ATOM | 3269 | NE | ARG | A | 489 | −61.144 | −77.815 | 91.128 | 1.00 | 200.66 | N |
| ATOM | 3270 | CZ | ARG | A | 489 | −61.927 | −78.892 | 91.021 | 1.00 | 200.14 | C |
| ATOM | 3271 | NH1 | ARG | A | 489 | −61.468 | −80.106 | 91.310 | 1.00 | 199.37 | N |
| ATOM | 3272 | NH2 | ARG | A | 489 | −63.183 | −78.756 | 90.624 | 1.00 | 199.89 | N |
| ATOM | 3273 | N | ARG | A | 490 | −59.549 | −77.836 | 97.779 | 1.00 | 204.55 | N |
| ATOM | 3274 | CA | ARG | A | 490 | −60.069 | −77.120 | 98.957 | 1.00 | 205.92 | C |
| ATOM | 3275 | C | ARG | A | 490 | −59.480 | −75.715 | 99.089 | 1.00 | 206.16 | C |
| ATOM | 3276 | O | ARG | A | 490 | −58.633 | −75.299 | 98.305 | 1.00 | 206.28 | O |
| ATOM | 3277 | CB | ARG | A | 490 | −59.773 | −77.912 | 100.253 | 1.00 | 206.00 | C |
| ATOM | 3278 | CG | ARG | A | 490 | −58.310 | −77.787 | 100.817 | 1.00 | 207.10 | C |
| ATOM | 3279 | CD | ARG | A | 490 | −58.220 | −77.918 | 102.372 | 1.00 | 207.30 | C |
| ATOM | 3280 | NE | ARG | A | 490 | −57.900 | −79.277 | 102.845 | 1.00 | 210.60 | N |
| ATOM | 3281 | CZ | ARG | A | 490 | −58.151 | −79.748 | 104.075 | 1.00 | 211.75 | C |
| ATOM | 3282 | NH1 | ARG | A | 490 | −58.747 | −78.985 | 104.993 | 1.00 | 212.65 | N |
| ATOM | 3283 | NH2 | ARG | A | 490 | −57.817 | −80.997 | 104.392 | 1.00 | 211.74 | N |
| ATOM | 3284 | N | LEU | A | 491 | −59.928 | −74.986 | 100.098 | 1.00 | 206.85 | N |
| ATOM | 3285 | CA | LEU | A | 491 | −59.196 | −73.818 | 100.551 | 1.00 | 207.58 | C |
| ATOM | 3286 | C | LEU | A | 491 | −59.086 | −73.937 | 102.068 | 1.00 | 208.00 | C |
| ATOM | 3287 | O | LEU | A | 491 | −60.115 | −74.061 | 102.741 | 1.00 | 207.93 | O |
| ATOM | 3288 | CB | LEU | A | 491 | −59.904 | −72.529 | 100.138 | 1.00 | 207.74 | C |
| ATOM | 3289 | CG | LEU | A | 491 | −60.091 | −72.261 | 98.641 | 1.00 | 208.14 | C |
| ATOM | 3290 | CD1 | LEU | A | 491 | −61.426 | −72.819 | 98.119 | 1.00 | 208.51 | C |
| ATOM | 3291 | CD2 | LEU | A | 491 | −60.001 | −70.773 | 98.391 | 1.00 | 208.32 | C |
| ATOM | 3292 | N | PRO | A | 492 | −57.845 | −73.905 | 102.611 | 1.00 | 208.42 | N |
| ATOM | 3293 | CA | PRO | A | 492 | −57.596 | −74.256 | 104.006 | 1.00 | 208.60 | C |
| ATOM | 3294 | C | PRO | A | 492 | −58.748 | −73.820 | 104.885 | 1.00 | 208.66 | C |
| ATOM | 3295 | O | PRO | A | 492 | −58.726 | −72.714 | 105.432 | 1.00 | 208.77 | O |
| ATOM | 3296 | CB | PRO | A | 492 | −56.320 | −73.469 | 104.345 | 1.00 | 208.60 | C |
| ATOM | 3297 | CG | PRO | A | 492 | −55.567 | −73.455 | 103.094 | 1.00 | 208.77 | C |
| ATOM | 3298 | CD | PRO | A | 492 | −56.595 | −73.495 | 101.944 | 1.00 | 208.65 | C |
| ATOM | 3299 | N | LYS | A | 493 | −59.762 | −74.680 | 104.966 | 1.00 | 208.65 | N |
| ATOM | 3300 | CA | LYS | A | 493 | −60.947 | −74.467 | 105.803 | 1.00 | 208.73 | C |
| ATOM | 3301 | C | LYS | A | 493 | −61.578 | −73.051 | 105.778 | 1.00 | 208.57 | C |
| ATOM | 3302 | O | LYS | A | 493 | −62.780 | −72.918 | 106.006 | 1.00 | 208.57 | O |
| ATOM | 3303 | CB | LYS | A | 493 | −60.670 | −74.917 | 107.257 | 1.00 | 208.88 | C |
| ATOM | 3304 | CG | LYS | A | 493 | −60.249 | −76.401 | 107.444 | 1.00 | 208.99 | C |
| ATOM | 3305 | CD | LYS | A | 493 | −61.412 | −77.411 | 107.283 | 1.00 | 208.97 | C |
| ATOM | 3306 | CE | LYS | A | 493 | −62.413 | −77.380 | 108.451 | 1.00 | 208.65 | C |
| ATOM | 3307 | NZ | LYS | A | 493 | −61.865 | −77.884 | 109.746 | 1.00 | 208.06 | N |
| ATOM | 3308 | N | GLY | A | 494 | −60.792 | −72.007 | 105.502 | 1.00 | 208.37 | N |
| ATOM | 3309 | CA | GLY | A | 494 | −61.263 | −70.638 | 105.714 | 1.00 | 208.22 | C |
| ATOM | 3310 | C | GLY | A | 494 | −60.824 | −69.526 | 104.777 | 1.00 | 208.20 | C |
| ATOM | 3311 | O | GLY | A | 494 | −61.606 | −68.614 | 104.506 | 1.00 | 208.22 | O |
| ATOM | 3312 | N | VAL | A | 495 | −59.589 | −69.587 | 104.279 | 1.00 | 208.18 | N |
| ATOM | 3313 | CA | VAL | A | 495 | −58.991 | −68.450 | 103.537 | 1.00 | 208.12 | C |
| ATOM | 3314 | C | VAL | A | 495 | −59.292 | −68.363 | 102.023 | 1.00 | 208.12 | C |
| ATOM | 3315 | O | VAL | A | 495 | −59.591 | −69.372 | 101.384 | 1.00 | 208.15 | O |
| ATOM | 3316 | CB | VAL | A | 495 | −57.459 | −68.326 | 103.791 | 1.00 | 208.12 | C |
| ATOM | 3317 | CG1 | VAL | A | 495 | −57.200 | −67.707 | 105.165 | 1.00 | 207.97 | C |
| ATOM | 3318 | CG2 | VAL | A | 495 | −56.738 | −69.677 | 103.617 | 1.00 | 207.85 | C |
| ATOM | 3319 | N | LYS | A | 496 | −59.191 | −67.153 | 101.465 | 1.00 | 208.06 | N |
| ATOM | 3320 | CA | LYS | A | 496 | −59.552 | −66.893 | 100.055 | 1.00 | 208.10 | C |
| ATOM | 3321 | C | LYS | A | 496 | −58.598 | −67.446 | 98.970 | 1.00 | 207.71 | C |
| ATOM | 3322 | O | LYS | A | 496 | −59.040 | −67.686 | 97.844 | 1.00 | 207.55 | O |
| ATOM | 3323 | CB | LYS | A | 496 | −59.868 | −65.402 | 99.816 | 1.00 | 208.44 | C |
| ATOM | 3324 | CG | LYS | A | 496 | −61.301 | −64.936 | 100.234 | 1.00 | 209.38 | C |
| ATOM | 3325 | CD | LYS | A | 496 | −62.449 | −65.585 | 99.409 | 1.00 | 210.31 | C |
| ATOM | 3326 | CE | LYS | A | 496 | −62.635 | −64.960 | 98.015 | 1.00 | 210.43 | C |
| ATOM | 3327 | NZ | LYS | A | 496 | −63.681 | −63.894 | 97.981 | 1.00 | 210.65 | N |
| ATOM | 3328 | N | HIS | A | 497 | −57.306 | −67.591 | 99.290 | 1.00 | 207.43 | N |
| ATOM | 3329 | CA | HIS | A | 497 | −56.354 | −68.401 | 98.478 | 1.00 | 207.29 | C |
| ATOM | 3330 | C | HIS | A | 497 | −54.894 | −68.503 | 99.011 | 1.00 | 206.91 | C |
| ATOM | 3331 | O | HIS | A | 497 | −54.315 | −67.528 | 99.489 | 1.00 | 206.75 | O |
| ATOM | 3332 | CB | HIS | A | 497 | −56.448 | −68.096 | 96.957 | 1.00 | 207.46 | C |
| ATOM | 3333 | CG | HIS | A | 497 | −55.332 | −67.261 | 96.407 | 1.00 | 208.03 | C |
| ATOM | 3334 | ND1 | HIS | A | 497 | −54.104 | −67.790 | 96.067 | 1.00 | 208.50 | N |
| ATOM | 3335 | CD2 | HIS | A | 497 | −55.275 | −65.947 | 96.084 | 1.00 | 208.56 | C |
| ATOM | 3336 | CE1 | HIS | A | 497 | −53.330 | −66.832 | 95.587 | 1.00 | 208.47 | C |
| ATOM | 3337 | NE2 | HIS | A | 497 | −54.016 | −65.703 | 95.587 | 1.00 | 208.70 | N |
| ATOM | 3338 | N | LEU | A | 498 | −54.323 | −69.701 | 98.875 | 1.00 | 206.47 | N |
| ATOM | 3339 | CA | LEU | A | 498 | −53.107 | −70.164 | 99.577 | 1.00 | 206.07 | C |
| ATOM | 3340 | C | LEU | A | 498 | −51.998 | −69.209 | 99.960 | 1.00 | 206.10 | C |
| ATOM | 3341 | O | LEU | A | 498 | −51.357 | −69.408 | 101.000 | 1.00 | 206.16 | O |
| ATOM | 3342 | CB | LEU | A | 498 | −52.480 | −71.317 | 98.825 | 1.00 | 205.74 | C |
| ATOM | 3343 | CG | LEU | A | 498 | −53.277 | −72.583 | 99.023 | 1.00 | 205.48 | C |
| ATOM | 3344 | CD1 | LEU | A | 498 | −54.384 | −72.693 | 97.975 | 1.00 | 205.07 | C |
| ATOM | 3345 | CD2 | LEU | A | 498 | −52.309 | −73.728 | 98.941 | 1.00 | 206.00 | C |
| ATOM | 3346 | N | LYS | A | 499 | −51.740 | −68.204 | 99.124 | 1.00 | 206.06 | N |
| ATOM | 3347 | CA | LYS | A | 499 | −50.764 | −67.184 | 99.491 | 1.00 | 206.21 | C |
| ATOM | 3348 | C | LYS | A | 499 | −51.345 | −66.286 | 100.581 | 1.00 | 206.31 | C |
| ATOM | 3349 | O | LYS | A | 499 | −50.944 | −65.140 | 100.757 | 1.00 | 206.14 | O |
| ATOM | 3350 | CB | LYS | A | 499 | −50.189 | −66.421 | 98.268 | 1.00 | 206.24 | C |
| ATOM | 3351 | CG | LYS | A | 499 | −51.096 | −65.459 | 97.477 | 1.00 | 206.38 | C |
| ATOM | 3352 | CD | LYS | A | 499 | −50.246 | −64.566 | 96.521 | 1.00 | 206.30 | C |
| ATOM | 3353 | CE | LYS | A | 499 | −51.061 | −63.860 | 95.409 | 1.00 | 206.77 | C |
| ATOM | 3354 | NZ | LYS | A | 499 | −51.473 | −62.435 | 95.684 | 1.00 | 207.20 | N |
| ATOM | 3355 | N | ASP | A | 500 | −52.273 | −66.858 | 101.342 | 1.00 | 206.71 | N |
| ATOM | 3356 | CA | ASP | A | 500 | −53.021 | −66.109 | 102.339 | 1.00 | 207.27 | C |
| ATOM | 3357 | C | ASP | A | 500 | −52.490 | −66.134 | 103.766 | 1.00 | 207.04 | C |
| ATOM | 3358 | O | ASP | A | 500 | −51.946 | −65.129 | 104.238 | 1.00 | 207.31 | O |
| ATOM | 3359 | CB | ASP | A | 500 | −54.499 | −66.498 | 102.332 | 1.00 | 207.73 | C |
| ATOM | 3360 | CG | ASP | A | 500 | −55.370 | −65.422 | 101.707 | 1.00 | 209.34 | C |
| ATOM | 3361 | OD1 | ASP | A | 500 | −56.374 | −65.002 | 102.346 | 1.00 | 211.00 | O |
| ATOM | 3362 | OD2 | ASP | A | 500 | −55.024 | −64.978 | 100.585 | 1.00 | 210.71 | O |
| ATOM | 3363 | N | PHE | A | 501 | −52.679 | −67.264 | 104.451 | 1.00 | 206.49 | N |
| ATOM | 3364 | CA | PHE | A | 501 | −52.408 | −67.365 | 105.899 | 1.00 | 205.82 | C |
| ATOM | 3365 | C | PHE | A | 501 | −51.723 | −68.699 | 106.320 | 1.00 | 204.51 | C |
| ATOM | 3366 | O | PHE | A | 501 | −51.103 | −68.757 | 107.400 | 1.00 | 204.23 | O |
| ATOM | 3367 | CB | PHE | A | 501 | −53.726 | −67.200 | 106.716 | 1.00 | 206.51 | C |
| ATOM | 3368 | CG | PHE | A | 501 | −54.140 | −65.749 | 107.041 | 1.00 | 207.26 | C |
| ATOM | 3369 | CD1 | PHE | A | 501 | −53.929 | −65.219 | 108.328 | 1.00 | 208.19 | C |
| ATOM | 3370 | CD2 | PHE | A | 501 | −54.819 | −64.958 | 106.099 | 1.00 | 207.49 | C |
| ATOM | 3371 | CE1 | PHE | A | 501 | −54.338 | −63.910 | 108.653 | 1.00 | 208.46 | C |
| ATOM | 3372 | CE2 | PHE | A | 501 | −55.228 | −63.648 | 106.413 | 1.00 | 207.87 | C |
| ATOM | 3373 | CZ | PHE | A | 501 | −54.987 | −63.125 | 107.692 | 1.00 | 207.97 | C |
| ATOM | 3374 | N | PRO | A | 502 | −51.833 | −69.765 | 105.481 | 1.00 | 203.22 | N |
| ATOM | 3375 | CA | PRO | A | 502 | −51.579 | −71.100 | 106.039 | 1.00 | 202.16 | C |
| ATOM | 3376 | C | PRO | A | 502 | −50.142 | −71.364 | 106.598 | 1.00 | 201.87 | C |
| ATOM | 3377 | O | PRO | A | 502 | −49.271 | −70.479 | 106.547 | 1.00 | 201.79 | O |
| ATOM | 3378 | CB | PRO | A | 502 | −51.963 | −72.042 | 104.873 | 1.00 | 202.31 | C |
| ATOM | 3379 | CG | PRO | A | 502 | −52.782 | −71.200 | 103.941 | 1.00 | 202.19 | C |
| ATOM | 3380 | CD | PRO | A | 502 | −52.177 | −69.848 | 104.048 | 1.00 | 202.79 | C |
| ATOM | 3381 | N | ILE | A | 503 | −49.952 | −72.562 | 107.172 | 1.00 | 201.01 | N |
| ATOM | 3382 | CA | ILE | A | 503 | −48.675 | −73.087 | 107.747 | 1.00 | 200.11 | C |
| ATOM | 3383 | C | ILE | A | 503 | −48.082 | −72.393 | 108.989 | 1.00 | 199.16 | C |
| ATOM | 3384 | O | ILE | A | 503 | −47.454 | −71.332 | 108.895 | 1.00 | 199.21 | O |
| ATOM | 3385 | CB | ILE | A | 503 | −47.606 | −73.353 | 106.674 | 1.00 | 199.68 | C |
| ATOM | 3386 | CG1 | ILE | A | 503 | −48.142 | −74.386 | 105.705 | 1.00 | 199.39 | C |
| ATOM | 3387 | CG2 | ILE | A | 503 | −46.318 | −73.866 | 107.294 | 1.00 | 199.19 | C |
| ATOM | 3388 | CD1 | ILE | A | 503 | −48.154 | −73.890 | 104.312 | 1.00 | 200.40 | C |
| ATOM | 3389 | N | LEU | A | 504 | −48.297 | −73.038 | 110.138 | 1.00 | 197.91 | N |
| ATOM | 3390 | CA | LEU | A | 504 | −47.787 | −72.630 | 111.439 | 1.00 | 196.68 | C |
| ATOM | 3391 | C | LEU | A | 504 | −46.440 | −73.288 | 111.664 | 1.00 | 196.07 | C |
| ATOM | 3392 | O | LEU | A | 504 | −46.015 | −74.094 | 110.845 | 1.00 | 195.94 | O |
| ATOM | 3393 | CB | LEU | A | 504 | −48.748 | −73.098 | 112.534 | 1.00 | 196.52 | C |
| ATOM | 3394 | CG | LEU | A | 504 | −50.071 | −72.366 | 112.748 | 1.00 | 195.95 | C |
| ATOM | 3395 | CD1 | LEU | A | 504 | −51.065 | −73.313 | 113.347 | 1.00 | 195.62 | C |
| ATOM | 3396 | CD2 | LEU | A | 504 | −49.897 | −71.152 | 113.642 | 1.00 | 195.49 | C |
| ATOM | 3397 | N | PRO | A | 505 | −45.763 | −72.956 | 112.780 | 1.00 | 195.54 | N |
| ATOM | 3398 | CA | PRO | A | 505 | −44.583 | −73.716 | 113.172 | 1.00 | 195.09 | C |
| ATOM | 3399 | C | PRO | A | 505 | −44.917 | −75.186 | 113.236 | 1.00 | 194.64 | C |
| ATOM | 3400 | O | PRO | A | 505 | −46.002 | −75.544 | 113.677 | 1.00 | 194.55 | O |
| ATOM | 3401 | CB | PRO | A | 505 | −44.290 | −73.209 | 114.589 | 1.00 | 195.16 | C |
| ATOM | 3402 | CG | PRO | A | 505 | −45.527 | −72.453 | 115.013 | 1.00 | 195.44 | C |
| ATOM | 3403 | CD | PRO | A | 505 | −46.041 | −71.876 | 113.739 | 1.00 | 195.60 | C |
| ATOM | 3404 | N | GLY | A | 506 | −44.000 | −76.025 | 112.776 | 1.00 | 194.37 | N |
| ATOM | 3405 | CA | GLY | A | 506 | −44.169 | −77.466 | 112.876 | 1.00 | 194.28 | C |
| ATOM | 3406 | C | GLY | A | 506 | −44.872 | −78.143 | 111.714 | 1.00 | 194.22 | C |
| ATOM | 3407 | O | GLY | A | 506 | −44.295 | −79.033 | 111.089 | 1.00 | 194.36 | O |
| ATOM | 3408 | N | GLU | A | 507 | −46.111 | −77.731 | 111.432 | 1.00 | 193.98 | N |
| ATOM | 3409 | CA | GLU | A | 507 | −46.964 | −78.394 | 110.428 | 1.00 | 193.96 | C |
| ATOM | 3410 | C | GLU | A | 507 | −46.405 | −78.337 | 108.993 | 1.00 | 193.22 | C |
| ATOM | 3411 | O | GLU | A | 507 | −45.718 | −77.387 | 108.623 | 1.00 | 193.08 | O |
| ATOM | 3412 | CB | GLU | A | 507 | −48.408 | −77.859 | 110.487 | 1.00 | 193.96 | C |
| ATOM | 3413 | CG | GLU | A | 507 | −48.528 | −76.311 | 110.424 | 1.00 | 195.28 | C |
| ATOM | 3414 | CD | GLU | A | 507 | −49.899 | −75.774 | 109.919 | 1.00 | 195.34 | C |
| ATOM | 3415 | OE1 | GLU | A | 507 | −50.579 | −76.445 | 109.099 | 1.00 | 197.24 | O |
| ATOM | 3416 | OE2 | GLU | A | 507 | −50.288 | −74.650 | 110.328 | 1.00 | 196.04 | O |
| ATOM | 3417 | N | ILE | A | 508 | −46.698 | −79.371 | 108.205 | 1.00 | 192.77 | N |
| ATOM | 3418 | CA | ILE | A | 508 | −46.237 | −79.495 | 106.810 | 1.00 | 192.39 | C |
| ATOM | 3419 | C | ILE | A | 508 | −47.427 | −79.502 | 105.848 | 1.00 | 192.06 | C |
| ATOM | 3420 | O | ILE | A | 508 | −48.490 | −80.051 | 106.177 | 1.00 | 192.21 | O |
| ATOM | 3421 | CB | ILE | A | 508 | −45.421 | −80.804 | 106.571 | 1.00 | 192.37 | C |
| ATOM | 3422 | CG1 | ILE | A | 508 | −46.293 | −82.057 | 106.806 | 1.00 | 192.79 | C |
| ATOM | 3423 | CG2 | ILE | A | 508 | −44.193 | −80.843 | 107.452 | 1.00 | 192.42 | C |
| ATOM | 3424 | CD1 | ILE | A | 508 | −45.613 | −83.411 | 106.525 | 1.00 | 192.67 | C |
| ATOM | 3425 | N | PHE | A | 509 | −47.247 | −78.906 | 104.665 | 1.00 | 191.51 | N |
| ATOM | 3426 | CA | PHE | A | 509 | −48.310 | −78.821 | 103.644 | 1.00 | 190.90 | C |
| ATOM | 3427 | C | PHE | A | 509 | −47.822 | −79.325 | 102.289 | 1.00 | 190.57 | C |
| ATOM | 3428 | O | PHE | A | 509 | −46.753 | −78.919 | 101.814 | 1.00 | 190.77 | O |
| ATOM | 3429 | CB | PHE | A | 509 | −48.783 | −77.372 | 103.484 | 1.00 | 190.85 | C |
| ATOM | 3430 | CG | PHE | A | 509 | −50.200 | −77.227 | 102.979 | 1.00 | 190.61 | C |
| ATOM | 3431 | CD1 | PHE | A | 509 | −50.836 | −78.256 | 102.289 | 1.00 | 190.39 | C |
| ATOM | 3432 | CD2 | PHE | A | 509 | −50.893 | −76.034 | 103.187 | 1.00 | 190.59 | C |
| ATOM | 3433 | CE1 | PHE | A | 509 | −52.135 | −78.103 | 101.834 | 1.00 | 190.83 | C |
| ATOM | 3434 | CE2 | PHE | A | 509 | −52.195 | −75.867 | 102.737 | 1.00 | 190.38 | C |
| ATOM | 3435 | CZ | PHE | A | 509 | −52.819 | −76.902 | 102.060 | 1.00 | 190.76 | C |
| ATOM | 3436 | N | LYS | A | 510 | −48.611 | −80.194 | 101.661 | 1.00 | 189.82 | N |
| ATOM | 3437 | CA | LYS | A | 510 | −48.270 | −80.691 | 100.337 | 1.00 | 189.17 | C |
| ATOM | 3438 | C | LYS | A | 510 | −48.950 | −79.831 | 99.260 | 1.00 | 188.79 | C |
| ATOM | 3439 | O | LYS | A | 510 | −50.173 | −79.848 | 99.129 | 1.00 | 188.93 | O |
| ATOM | 3440 | CB | LYS | A | 510 | −48.646 | −82.168 | 100.211 | 1.00 | 189.07 | C |
| ATOM | 3441 | CG | LYS | A | 510 | −48.064 | −83.075 | 101.303 | 1.00 | 188.98 | C |
| ATOM | 3442 | CD | LYS | A | 510 | −48.403 | −84.543 | 101.036 | 1.00 | 189.31 | C |
| ATOM | 3443 | CE | LYS | A | 510 | −47.884 | −85.491 | 102.111 | 1.00 | 189.49 | C |
| ATOM | 3444 | NZ | LYS | A | 510 | −47.821 | −86.907 | 101.611 | 1.00 | 189.76 | N |
| ATOM | 3445 | N | TYR | A | 511 | −48.150 | −79.063 | 98.517 | 1.00 | 188.10 | N |
| ATOM | 3446 | CA | TYR | A | 511 | −48.645 | −78.160 | 97.483 | 1.00 | 187.47 | C |
| ATOM | 3447 | C | TYR | A | 511 | −48.475 | −78.796 | 96.106 | 1.00 | 187.47 | C |
| ATOM | 3448 | O | TYR | A | 511 | −47.536 | −79.556 | 95.897 | 1.00 | 187.29 | O |
| ATOM | 3449 | CB | TYR | A | 511 | −47.818 | −76.885 | 97.491 | 1.00 | 187.15 | C |
| ATOM | 3450 | CG | TYR | A | 511 | −48.107 | −75.834 | 98.548 | 1.00 | 186.97 | C |
| ATOM | 3451 | CD1 | TYR | A | 511 | −48.091 | −76.127 | 99.913 | 1.00 | 186.45 | C |
| ATOM | 3452 | CD2 | TYR | A | 511 | −48.311 | −74.511 | 98.172 | 1.00 | 187.17 | C |
| ATOM | 3453 | CE1 | TYR | A | 511 | −48.319 | −75.128 | 100.866 | 1.00 | 185.95 | C |
| ATOM | 3454 | CE2 | TYR | A | 511 | −48.538 | −73.516 | 99.115 | 1.00 | 187.09 | C |
| ATOM | 3455 | CZ | TYR | A | 511 | −48.541 | −73.824 | 100.454 | 1.00 | 186.48 | C |
| ATOM | 3456 | OH | TYR | A | 511 | −48.771 | −72.803 | 101.351 | 1.00 | 186.51 | O |
| ATOM | 3457 | N | LYS | A | 512 | −49.369 | −78.476 | 95.166 | 1.00 | 187.72 | N |
| ATOM | 3458 | CA | LYS | A | 512 | −49.214 | −78.869 | 93.745 | 1.00 | 187.85 | C |
| ATOM | 3459 | C | LYS | A | 512 | −49.146 | −77.662 | 92.808 | 1.00 | 187.96 | C |
| ATOM | 3460 | O | LYS | A | 512 | −50.084 | −76.861 | 92.705 | 1.00 | 187.59 | O |
| ATOM | 3461 | CB | LYS | A | 512 | −50.314 | −79.839 | 93.275 | 1.00 | 187.86 | C |
| ATOM | 3462 | CG | LYS | A | 512 | −50.225 | −80.268 | 91.795 | 1.00 | 187.69 | C |
| ATOM | 3463 | CD | LYS | A | 512 | −51.462 | −81.077 | 91.364 | 1.00 | 188.10 | C |
| ATOM | 3464 | CE | LYS | A | 512 | −51.430 | −81.551 | 89.886 | 1.00 | 188.72 | C |
| ATOM | 3465 | NZ | LYS | A | 512 | −50.954 | −82.965 | 89.633 | 1.00 | 189.26 | N |
| ATOM | 3466 | N | TRP | A | 513 | −48.003 | −77.561 | 92.136 | 1.00 | 188.45 | N |
| ATOM | 3467 | CA | TRP | A | 513 | −47.763 | −76.589 | 91.075 | 1.00 | 188.92 | C |
| ATOM | 3468 | C | TRP | A | 513 | −47.767 | −77.292 | 89.697 | 1.00 | 189.25 | C |
| ATOM | 3469 | O | TRP | A | 513 | −46.845 | −78.060 | 89.375 | 1.00 | 189.49 | O |
| ATOM | 3470 | CB | TRP | A | 513 | −46.429 | −75.851 | 91.301 | 1.00 | 188.75 | C |
| ATOM | 3471 | CG | TRP | A | 513 | −46.350 | −75.045 | 92.577 | 1.00 | 188.70 | C |
| ATOM | 3472 | CD1 | TRP | A | 513 | −47.389 | −74.650 | 93.371 | 1.00 | 188.80 | C |
| ATOM | 3473 | CD2 | TRP | A | 513 | −45.166 | −74.512 | 93.180 | 1.00 | 188.43 | C |
| ATOM | 3474 | NE1 | TRP | A | 513 | −46.923 | −73.915 | 94.438 | 1.00 | 188.76 | N |
| ATOM | 3475 | CE2 | TRP | A | 513 | −45.562 | −73.819 | 94.343 | 1.00 | 188.47 | C |
| ATOM | 3476 | CE3 | TRP | A | 513 | −43.810 | −74.561 | 92.855 | 1.00 | 188.46 | C |
| ATOM | 3477 | CZ2 | TRP | A | 513 | −44.653 | −73.179 | 95.173 | 1.00 | 188.82 | C |
| ATOM | 3478 | CZ3 | TRP | A | 513 | −42.911 | −73.921 | 93.676 | 1.00 | 188.57 | C |
| ATOM | 3479 | CH2 | TRP | A | 513 | −43.333 | −73.236 | 94.819 | 1.00 | 188.92 | C |
| ATOM | 3480 | N | THR | A | 514 | −48.814 | −77.036 | 88.904 | 1.00 | 189.18 | N |
| ATOM | 3481 | CA | THR | A | 514 | −48.930 | −77.561 | 87.546 | 1.00 | 188.91 | C |
| ATOM | 3482 | C | THR | A | 514 | −48.650 | −76.439 | 86.548 | 1.00 | 188.87 | C |
| ATOM | 3483 | O | THR | A | 514 | −49.243 | −75.363 | 86.628 | 1.00 | 188.65 | O |
| ATOM | 3484 | CB | THR | A | 514 | −50.320 | −78.150 | 87.294 | 1.00 | 188.88 | C |
| ATOM | 3485 | OG1 | THR | A | 514 | −50.639 | −79.074 | 88.338 | 1.00 | 188.85 | O |
| ATOM | 3486 | CG2 | THR | A | 514 | −50.357 | −78.882 | 85.963 | 1.00 | 189.31 | C |
| ATOM | 3487 | N | VAL | A | 515 | −47.746 | −76.705 | 85.607 | 1.00 | 188.90 | N |
| ATOM | 3488 | CA | VAL | A | 515 | −47.263 | −75.680 | 84.694 | 1.00 | 188.85 | C |
| ATOM | 3489 | C | VAL | A | 515 | −47.740 | −75.922 | 83.294 | 1.00 | 188.83 | C |
| ATOM | 3490 | O | VAL | A | 515 | −47.540 | −76.999 | 82.741 | 1.00 | 188.69 | O |
| ATOM | 3491 | CB | VAL | A | 515 | −45.753 | −75.662 | 84.612 | 1.00 | 188.90 | C |
| ATOM | 3492 | CG1 | VAL | A | 515 | −45.273 | −74.241 | 84.743 | 1.00 | 189.22 | C |
| ATOM | 3493 | CG2 | VAL | A | 515 | −45.138 | −76.540 | 85.688 | 1.00 | 189.09 | C |
| ATOM | 3494 | N | THR | A | 516 | −48.340 | −74.894 | 82.715 | 1.00 | 189.07 | N |
| ATOM | 3495 | CA | THR | A | 516 | −48.970 | −75.004 | 81.408 | 1.00 | 189.64 | C |
| ATOM | 3496 | C | THR | A | 516 | −48.399 | −73.986 | 80.443 | 1.00 | 189.79 | C |
| ATOM | 3497 | O | THR | A | 516 | −47.681 | −73.083 | 80.854 | 1.00 | 189.82 | O |
| ATOM | 3498 | CB | THR | A | 516 | −50.485 | −74.832 | 81.509 | 1.00 | 189.69 | C |
| ATOM | 3499 | OG1 | THR | A | 516 | −50.786 | −73.734 | 82.380 | 1.00 | 190.01 | O |
| ATOM | 3500 | CG2 | THR | A | 516 | −51.125 | −76.099 | 82.065 | 1.00 | 190.38 | C |
| ATOM | 3501 | N | VAL | A | 517 | −48.728 | −74.121 | 79.160 | 1.00 | 190.11 | N |
| ATOM | 3502 | CA | VAL | A | 517 | −48.012 | −73.380 | 78.112 | 1.00 | 190.58 | C |
| ATOM | 3503 | C | VAL | A | 517 | −48.064 | −71.876 | 78.314 | 1.00 | 190.86 | C |
| ATOM | 3504 | O | VAL | A | 517 | −47.161 | −71.146 | 77.894 | 1.00 | 191.14 | O |
| ATOM | 3505 | CB | VAL | A | 517 | −48.494 | −73.749 | 76.687 | 1.00 | 190.51 | C |
| ATOM | 3506 | CG1 | VAL | A | 517 | −48.926 | −75.197 | 76.647 | 1.00 | 190.94 | C |
| ATOM | 3507 | CG2 | VAL | A | 517 | −49.632 | −72.836 | 76.207 | 1.00 | 190.74 | C |
| ATOM | 3508 | N | GLU | A | 518 | −49.119 | −71.433 | 78.985 | 1.00 | 191.03 | N |
| ATOM | 3509 | CA | GLU | A | 518 | −49.423 | −70.023 | 79.117 | 1.00 | 191.26 | C |
| ATOM | 3510 | C | GLU | A | 518 | −48.417 | −69.294 | 79.990 | 1.00 | 190.81 | C |
| ATOM | 3511 | O | GLU | A | 518 | −48.125 | −68.132 | 79.729 | 1.00 | 190.73 | O |
| ATOM | 3512 | CB | GLU | A | 518 | −50.851 | −69.832 | 79.637 | 1.00 | 191.65 | C |
| ATOM | 3513 | CG | GLU | A | 518 | −51.362 | −70.951 | 80.574 | 1.00 | 193.76 | C |
| ATOM | 3514 | CD | GLU | A | 518 | −51.956 | −72.177 | 79.839 | 1.00 | 196.20 | C |
| ATOM | 3515 | OE1 | GLU | A | 518 | −52.684 | −72.962 | 80.500 | 1.00 | 197.01 | O |
| ATOM | 3516 | OE2 | GLU | A | 518 | −51.704 | −72.359 | 78.619 | 1.00 | 196.67 | O |
| ATOM | 3517 | N | ASP | A | 519 | −47.884 | −69.982 | 81.003 | 1.00 | 190.42 | N |
| ATOM | 3518 | CA | ASP | A | 519 | −46.921 | −69.385 | 81.949 | 1.00 | 190.28 | C |
| ATOM | 3519 | C | ASP | A | 519 | −45.462 | −69.694 | 81.642 | 1.00 | 189.58 | C |
| ATOM | 3520 | O | ASP | A | 519 | −44.559 | −69.328 | 82.394 | 1.00 | 189.31 | O |
| ATOM | 3521 | CB | ASP | A | 519 | −47.243 | −69.725 | 83.417 | 1.00 | 190.73 | C |
| ATOM | 3522 | CG | ASP | A | 519 | −48.330 | −70.786 | 83.572 | 1.00 | 192.39 | C |
| ATOM | 3523 | OD1 | ASP | A | 519 | −49.272 | −70.549 | 84.372 | 1.00 | 194.05 | O |
| ATOM | 3524 | OD2 | ASP | A | 519 | −48.245 | −71.854 | 82.918 | 1.00 | 194.21 | O |
| ATOM | 3525 | N | GLY | A | 520 | −45.247 | −70.365 | 80.523 | 1.00 | 189.11 | N |
| ATOM | 3526 | CA | GLY | A | 520 | −43.908 | −70.628 | 80.040 | 1.00 | 188.66 | C |
| ATOM | 3527 | C | GLY | A | 520 | −43.575 | −69.632 | 78.792 | 1.00 | 188.33 | C |
| ATOM | 3528 | O | GLY | A | 520 | −44.428 | −69.108 | 78.258 | 1.00 | 188.41 | O |
| ATOM | 3529 | N | PRO | A | 521 | −42.339 | −69.990 | 78.293 | 1.00 | 187.99 | N |
| ATOM | 3530 | CA | PRO | A | 521 | −41.863 | −69.152 | 77.219 | 1.00 | 187.72 | C |
| ATOM | 3531 | C | PRO | A | 521 | −42.645 | −69.417 | 75.962 | 1.00 | 187.39 | C |
| ATOM | 3532 | O | PRO | A | 521 | −43.759 | −69.935 | 76.015 | 1.00 | 187.09 | O |
| ATOM | 3533 | CB | PRO | A | 521 | −40.413 | −69.609 | 77.045 | 1.00 | 187.94 | C |
| ATOM | 3534 | CG | PRO | A | 521 | −40.409 | −71.016 | 77.518 | 1.00 | 188.00 | C |
| ATOM | 3535 | CD | PRO | A | 521 | −41.333 | −70.994 | 78.683 | 1.00 | 188.03 | C |
| ATOM | 3536 | N | THR | A | 522 | −42.051 | −69.069 | 74.832 | 1.00 | 187.33 | N |
| ATOM | 3537 | CA | THR | A | 522 | −42.732 | −69.200 | 73.572 | 1.00 | 187.29 | C |
| ATOM | 3538 | C | THR | A | 522 | −41.767 | −69.297 | 72.408 | 1.00 | 187.54 | C |
| ATOM | 3539 | O | THR | A | 522 | −40.548 | −69.429 | 72.586 | 1.00 | 187.57 | O |
| ATOM | 3540 | CB | THR | A | 522 | −43.593 | −67.999 | 73.348 | 1.00 | 187.13 | C |
| ATOM | 3541 | OG1 | THR | A | 522 | −42.745 | −66.856 | 73.195 | 1.00 | 186.60 | O |
| ATOM | 3542 | CG2 | THR | A | 522 | −44.539 | −67.802 | 74.535 | 1.00 | 186.82 | C |
| ATOM | 3543 | N | LYS | A | 523 | −42.329 | −69.216 | 71.208 | 1.00 | 187.76 | N |
| ATOM | 3544 | CA | LYS | A | 523 | −41.531 | −69.318 | 70.012 | 1.00 | 188.11 | C |
| ATOM | 3545 | C | LYS | A | 523 | −40.733 | −68.035 | 69.825 | 1.00 | 188.39 | C |
| ATOM | 3546 | O | LYS | A | 523 | −40.038 | −67.870 | 68.830 | 1.00 | 188.65 | O |
| ATOM | 3547 | CB | LYS | A | 523 | −42.388 | −69.693 | 68.785 | 1.00 | 188.02 | C |
| ATOM | 3548 | CG | LYS | A | 523 | −43.492 | −68.710 | 68.389 | 1.00 | 188.07 | C |
| ATOM | 3549 | CD | LYS | A | 523 | −44.271 | −69.214 | 67.163 | 1.00 | 188.17 | C |
| ATOM | 3550 | CE | LYS | A | 523 | −44.896 | −68.073 | 66.338 | 1.00 | 188.28 | C |
| ATOM | 3551 | NZ | LYS | A | 523 | −43.986 | −67.531 | 65.276 | 1.00 | 187.54 | N |
| ATOM | 3552 | N | SER | A | 524 | −40.805 | −67.140 | 70.802 | 1.00 | 188.70 | N |
| ATOM | 3553 | CA | SER | A | 524 | −40.050 | −65.901 | 70.713 | 1.00 | 189.36 | C |
| ATOM | 3554 | C | SER | A | 524 | −39.021 | −65.705 | 71.810 | 1.00 | 189.71 | C |
| ATOM | 3555 | O | SER | A | 524 | −37.873 | −65.374 | 71.527 | 1.00 | 189.79 | O |
| ATOM | 3556 | CB | SER | A | 524 | −40.984 | −64.697 | 70.652 | 1.00 | 189.42 | C |
| ATOM | 3557 | OG | SER | A | 524 | −41.007 | −64.152 | 69.342 | 1.00 | 190.15 | O |
| ATOM | 3558 | N | ASP | A | 525 | −39.440 | −65.916 | 73.054 | 1.00 | 190.28 | N |
| ATOM | 3559 | CA | ASP | A | 525 | −38.653 | −65.602 | 74.254 | 1.00 | 190.65 | C |
| ATOM | 3560 | C | ASP | A | 525 | −37.271 | −66.241 | 74.276 | 1.00 | 190.87 | C |
| ATOM | 3561 | O | ASP | A | 525 | −36.705 | −66.572 | 73.240 | 1.00 | 190.95 | O |
| ATOM | 3562 | CB | ASP | A | 525 | −39.424 | −66.073 | 75.492 | 1.00 | 190.62 | C |
| ATOM | 3563 | CG | ASP | A | 525 | −40.865 | −65.631 | 75.483 | 1.00 | 191.14 | C |
| ATOM | 3564 | OD1 | ASP | A | 525 | −41.106 | −64.405 | 75.381 | 1.00 | 192.14 | O |
| ATOM | 3565 | OD2 | ASP | A | 525 | −41.751 | −66.507 | 75.574 | 1.00 | 190.66 | O |
| ATOM | 3566 | N | PRO | A | 526 | −36.703 | −66.389 | 75.471 | 1.00 | 191.18 | N |
| ATOM | 3567 | CA | PRO | A | 526 | −35.700 | −67.418 | 75.612 | 1.00 | 191.65 | C |
| ATOM | 3568 | C | PRO | A | 526 | −36.444 | −68.729 | 75.673 | 1.00 | 192.18 | C |
| ATOM | 3569 | O | PRO | A | 526 | −37.611 | −68.794 | 75.285 | 1.00 | 192.09 | O |
| ATOM | 3570 | CB | PRO | A | 526 | −35.099 | −67.122 | 76.974 | 1.00 | 191.64 | C |
| ATOM | 3571 | CG | PRO | A | 526 | −36.200 | −66.478 | 77.716 | 1.00 | 191.56 | C |
| ATOM | 3572 | CD | PRO | A | 526 | −36.900 | −65.635 | 76.717 | 1.00 | 191.15 | C |
| ATOM | 3573 | N | ARG | A | 527 | −35.789 | −69.769 | 76.163 | 1.00 | 192.95 | N |
| ATOM | 3574 | CA | ARG | A | 527 | −36.486 | −71.022 | 76.336 | 1.00 | 193.84 | C |
| ATOM | 3575 | C | ARG | A | 527 | −36.738 | −71.249 | 77.812 | 1.00 | 193.81 | C |
| ATOM | 3576 | O | ARG | A | 527 | −37.431 | −72.184 | 78.194 | 1.00 | 193.90 | O |
| ATOM | 3577 | CB | ARG | A | 527 | −35.758 | −72.185 | 75.639 | 1.00 | 194.20 | C |
| ATOM | 3578 | CG | ARG | A | 527 | −35.704 | −72.010 | 74.094 | 1.00 | 196.14 | C |
| ATOM | 3579 | CD | ARG | A | 527 | −36.131 | −73.254 | 73.292 | 1.00 | 198.99 | C |
| ATOM | 3580 | NE | ARG | A | 527 | −35.183 | −74.373 | 73.391 | 1.00 | 201.47 | N |
| ATOM | 3581 | CZ | ARG | A | 527 | −34.306 | −74.731 | 72.446 | 1.00 | 202.58 | C |
| ATOM | 3582 | NH1 | ARG | A | 527 | −34.226 | −74.069 | 71.293 | 1.00 | 202.82 | N |
| ATOM | 3583 | NH2 | ARG | A | 527 | −33.494 | −75.764 | 72.660 | 1.00 | 203.06 | N |
| ATOM | 3584 | N | CYS | A | 528 | −36.224 | −70.346 | 78.636 | 1.00 | 193.93 | N |
| ATOM | 3585 | CA | CYS | A | 528 | −36.380 | −70.481 | 80.071 | 1.00 | 194.46 | C |
| ATOM | 3586 | C | CYS | A | 528 | −36.580 | −69.164 | 80.774 | 1.00 | 193.91 | C |
| ATOM | 3587 | O | CYS | A | 528 | −35.600 | −68.551 | 81.196 | 1.00 | 194.03 | O |
| ATOM | 3588 | CB | CYS | A | 528 | −35.136 | −71.133 | 80.652 | 1.00 | 194.92 | C |
| ATOM | 3589 | SG | CYS | A | 528 | −35.155 | −72.912 | 80.572 | 1.00 | 198.51 | S |
| ATOM | 3590 | N | LEU | A | 529 | −37.827 | −68.726 | 80.934 | 1.00 | 193.35 | N |
| ATOM | 3591 | CA | LEU | A | 529 | −38.056 | −67.432 | 81.588 | 1.00 | 192.87 | C |
| ATOM | 3592 | C | LEU | A | 529 | −37.739 | −67.474 | 83.077 | 1.00 | 193.10 | C |
| ATOM | 3593 | O | LEU | A | 529 | −37.824 | −68.537 | 83.712 | 1.00 | 193.13 | O |
| ATOM | 3594 | CB | LEU | A | 529 | −39.445 | −66.855 | 81.312 | 1.00 | 192.34 | C |
| ATOM | 3595 | CG | LEU | A | 529 | −40.480 | −67.681 | 80.569 | 1.00 | 191.19 | C |
| ATOM | 3596 | CD1 | LEU | A | 529 | −41.101 | −68.679 | 81.487 | 1.00 | 190.67 | C |
| ATOM | 3597 | CD2 | LEU | A | 529 | −41.529 | −66.747 | 80.042 | 1.00 | 190.82 | C |
| ATOM | 3598 | N | THR | A | 530 | −37.342 | −66.325 | 83.619 | 1.00 | 193.16 | N |
| ATOM | 3599 | CA | THR | A | 530 | −36.908 | −66.269 | 85.009 | 1.00 | 193.07 | C |
| ATOM | 3600 | C | THR | A | 530 | −37.981 | −65.762 | 85.944 | 1.00 | 192.95 | C |
| ATOM | 3601 | O | THR | A | 530 | −38.657 | −64.774 | 85.680 | 1.00 | 192.61 | O |
| ATOM | 3602 | CB | THR | A | 530 | −35.588 | −65.506 | 85.170 | 1.00 | 193.15 | C |
| ATOM | 3603 | OG1 | THR | A | 530 | −34.514 | −66.386 | 84.821 | 1.00 | 193.25 | O |
| ATOM | 3604 | CG2 | THR | A | 530 | −35.389 | −65.036 | 86.609 | 1.00 | 193.02 | C |
| ATOM | 3605 | N | ARG | A | 531 | −38.126 | −66.491 | 87.035 | 1.00 | 193.18 | N |
| ATOM | 3606 | CA | ARG | A | 531 | −39.144 | −66.234 | 88.024 | 1.00 | 193.84 | C |
| ATOM | 3607 | C | ARG | A | 531 | −38.600 | −66.687 | 89.382 | 1.00 | 194.30 | C |
| ATOM | 3608 | O | ARG | A | 531 | −37.431 | −67.056 | 89.481 | 1.00 | 194.41 | O |
| ATOM | 3609 | CB | ARG | A | 531 | −40.427 | −67.010 | 87.687 | 1.00 | 193.99 | C |
| ATOM | 3610 | CG | ARG | A | 531 | −41.072 | −66.712 | 86.349 | 1.00 | 193.38 | C |
| ATOM | 3611 | CD | ARG | A | 531 | −41.762 | −65.396 | 86.368 | 1.00 | 193.41 | C |
| ATOM | 3612 | NE | ARG | A | 531 | −41.486 | −64.687 | 85.131 | 1.00 | 194.84 | N |
| ATOM | 3613 | CZ | ARG | A | 531 | −42.267 | −64.715 | 84.054 | 1.00 | 195.58 | C |
| ATOM | 3614 | NH1 | ARG | A | 531 | −43.401 | −65.420 | 84.049 | 1.00 | 195.05 | N |
| ATOM | 3615 | NH2 | ARG | A | 531 | −41.908 | −64.024 | 82.977 | 1.00 | 196.06 | N |
| ATOM | 3616 | N | TYR | A | 532 | −39.450 | −66.688 | 90.411 | 1.00 | 194.71 | N |
| ATOM | 3617 | CA | TYR | A | 532 | −39.012 | −66.907 | 91.780 | 1.00 | 195.11 | C |
| ATOM | 3618 | C | TYR | A | 532 | −40.171 | −67.273 | 92.670 | 1.00 | 195.51 | C |
| ATOM | 3619 | O | TYR | A | 532 | −41.329 | −67.049 | 92.293 | 1.00 | 195.45 | O |
| ATOM | 3620 | CB | TYR | A | 532 | −38.411 | −65.620 | 92.333 | 1.00 | 195.25 | C |
| ATOM | 3621 | CG | TYR | A | 532 | −39.420 | −64.512 | 92.528 | 1.00 | 194.95 | C |
| ATOM | 3622 | CD1 | TYR | A | 532 | −40.032 | −64.305 | 93.758 | 1.00 | 194.86 | C |
| ATOM | 3623 | CD2 | TYR | A | 532 | −39.754 | −63.672 | 91.483 | 1.00 | 194.81 | C |
| ATOM | 3624 | CE1 | TYR | A | 532 | −40.949 | −63.289 | 93.936 | 1.00 | 195.38 | C |
| ATOM | 3625 | CE2 | TYR | A | 532 | −40.665 | −62.653 | 91.645 | 1.00 | 195.34 | C |
| ATOM | 3626 | CZ | TYR | A | 532 | −41.261 | −62.462 | 92.869 | 1.00 | 195.72 | C |
| ATOM | 3627 | OH | TYR | A | 532 | −42.175 | −61.436 | 93.009 | 1.00 | 196.45 | O |
| ATOM | 3628 | N | TYR | A | 533 | −39.840 | −67.800 | 93.858 | 1.00 | 195.97 | N |
| ATOM | 3629 | CA | TYR | A | 533 | −40.798 | −67.957 | 94.965 | 1.00 | 196.42 | C |
| ATOM | 3630 | C | TYR | A | 533 | −40.386 | −67.183 | 96.224 | 1.00 | 197.13 | C |
| ATOM | 3631 | O | TYR | A | 533 | −39.243 | −67.252 | 96.674 | 1.00 | 196.95 | O |
| ATOM | 3632 | CB | TYR | A | 533 | −41.097 | −69.437 | 95.280 | 1.00 | 195.79 | C |
| ATOM | 3633 | CG | TYR | A | 533 | −39.883 | −70.280 | 95.546 | 1.00 | 195.18 | C |
| ATOM | 3634 | CD1 | TYR | A | 533 | −39.328 | −70.353 | 96.811 | 1.00 | 195.72 | C |
| ATOM | 3635 | CD2 | TYR | A | 533 | −39.293 | −71.019 | 94.539 | 1.00 | 194.90 | C |
| ATOM | 3636 | CE1 | TYR | A | 533 | −38.200 | −71.135 | 97.065 | 1.00 | 195.50 | C |
| ATOM | 3637 | CE2 | TYR | A | 533 | −38.163 | −71.802 | 94.780 | 1.00 | 194.82 | C |
| ATOM | 3638 | CZ | TYR | A | 533 | −37.625 | −71.857 | 96.044 | 1.00 | 194.95 | C |
| ATOM | 3639 | OH | TYR | A | 533 | −36.514 | −72.624 | 96.300 | 1.00 | 194.63 | O |
| ATOM | 3640 | N | SER | A | 534 | −41.332 | −66.406 | 96.745 | 1.00 | 198.28 | N |
| ATOM | 3641 | CA | SER | A | 534 | −41.208 | −65.735 | 98.031 | 1.00 | 199.52 | C |
| ATOM | 3642 | C | SER | A | 534 | −42.060 | −66.568 | 98.983 | 1.00 | 200.38 | C |
| ATOM | 3643 | O | SER | A | 534 | −42.621 | −67.591 | 98.571 | 1.00 | 200.52 | O |
| ATOM | 3644 | CB | SER | A | 534 | −41.749 | −64.294 | 97.922 | 1.00 | 199.52 | C |
| ATOM | 3645 | OG | SER | A | 534 | −41.617 | −63.559 | 99.135 | 1.00 | 199.50 | O |
| ATOM | 3646 | N | SER | A | 535 | −42.143 | −66.156 | 100.248 | 1.00 | 201.40 | N |
| ATOM | 3647 | CA | SER | A | 535 | −43.286 | −66.553 | 101.071 | 1.00 | 202.35 | C |
| ATOM | 3648 | C | SER | A | 535 | −44.214 | −65.372 | 101.235 | 1.00 | 203.23 | C |
| ATOM | 3649 | O | SER | A | 535 | −43.795 | −64.224 | 101.069 | 1.00 | 203.42 | O |
| ATOM | 3650 | CB | SER | A | 535 | −42.894 | −67.063 | 102.439 | 1.00 | 202.24 | C |
| ATOM | 3651 | OG | SER | A | 535 | −44.082 | −67.399 | 103.137 | 1.00 | 201.75 | O |
| ATOM | 3652 | N | PHE | A | 536 | −45.470 | −65.654 | 101.578 | 1.00 | 204.32 | N |
| ATOM | 3653 | CA | PHE | A | 536 | −46.528 | −64.635 | 101.501 | 1.00 | 205.38 | C |
| ATOM | 3654 | C | PHE | A | 536 | −47.434 | −64.447 | 102.742 | 1.00 | 205.94 | C |
| ATOM | 3655 | O | PHE | A | 536 | −48.515 | −63.843 | 102.631 | 1.00 | 206.03 | O |
| ATOM | 3656 | CB | PHE | A | 536 | −47.389 | −64.867 | 100.247 | 1.00 | 205.52 | C |
| ATOM | 3657 | CG | PHE | A | 536 | −46.789 | −64.325 | 98.987 | 1.00 | 205.45 | C |
| ATOM | 3658 | CD1 | PHE | A | 536 | −47.339 | −63.209 | 98.378 | 1.00 | 205.58 | C |
| ATOM | 3659 | CD2 | PHE | A | 536 | −45.681 | −64.937 | 98.404 | 1.00 | 205.97 | C |
| ATOM | 3660 | CE1 | PHE | A | 536 | −46.796 | −62.701 | 97.210 | 1.00 | 206.71 | C |
| ATOM | 3661 | CE2 | PHE | A | 536 | −45.121 | −64.440 | 97.240 | 1.00 | 206.66 | C |
| ATOM | 3662 | CZ | PHE | A | 536 | −45.677 | −63.316 | 96.637 | 1.00 | 206.99 | C |
| ATOM | 3663 | N | VAL | A | 537 | −47.009 | −64.945 | 103.908 | 1.00 | 206.43 | N |
| ATOM | 3664 | CA | VAL | A | 537 | −47.735 | −64.642 | 105.147 | 1.00 | 206.75 | C |
| ATOM | 3665 | C | VAL | A | 537 | −47.642 | −63.121 | 105.406 | 1.00 | 207.08 | C |
| ATOM | 3666 | O | VAL | A | 537 | −48.671 | −62.461 | 105.586 | 1.00 | 207.27 | O |
| ATOM | 3667 | CB | VAL | A | 537 | −47.322 | −65.565 | 106.350 | 1.00 | 206.64 | C |
| ATOM | 3668 | CG1 | VAL | A | 537 | −45.922 | −65.276 | 106.814 | 1.00 | 206.74 | C |
| ATOM | 3669 | CG2 | VAL | A | 537 | −48.315 | −65.456 | 107.517 | 1.00 | 206.75 | C |
| ATOM | 3670 | N | ASN | A | 538 | −46.425 | −62.568 | 105.374 | 1.00 | 207.38 | N |
| ATOM | 3671 | CA | ASN | A | 538 | −46.223 | −61.114 | 105.231 | 1.00 | 207.75 | C |
| ATOM | 3672 | C | ASN | A | 538 | −45.455 | −60.864 | 103.935 | 1.00 | 207.61 | C |
| ATOM | 3673 | O | ASN | A | 538 | −44.872 | −61.800 | 103.390 | 1.00 | 207.66 | O |
| ATOM | 3674 | CB | ASN | A | 538 | −45.492 | −60.510 | 106.442 | 1.00 | 207.98 | C |
| ATOM | 3675 | CG | ASN | A | 538 | −45.837 | −59.024 | 106.670 | 1.00 | 208.80 | C |
| ATOM | 3676 | OD1 | ASN | A | 538 | −45.695 | −58.186 | 105.769 | 1.00 | 209.97 | O |
| ATOM | 3677 | ND2 | ASN | A | 538 | −46.271 | −58.697 | 107.889 | 1.00 | 209.05 | N |
| ATOM | 3678 | N | MET | A | 539 | −45.456 | −59.634 | 103.418 | 1.00 | 207.42 | N |
| ATOM | 3679 | CA | MET | A | 539 | −44.827 | −59.436 | 102.123 | 1.00 | 207.21 | C |
| ATOM | 3680 | C | MET | A | 539 | −43.300 | −59.456 | 102.155 | 1.00 | 206.97 | C |
| ATOM | 3681 | O | MET | A | 539 | −42.679 | −60.366 | 101.607 | 1.00 | 206.96 | O |
| ATOM | 3682 | CB | MET | A | 539 | −45.382 | −58.246 | 101.345 | 1.00 | 207.28 | C |
| ATOM | 3683 | CG | MET | A | 539 | −45.082 | −58.417 | 99.849 | 1.00 | 208.04 | C |
| ATOM | 3684 | SD | MET | A | 539 | −45.006 | −60.183 | 99.352 | 1.00 | 209.24 | S |
| ATOM | 3685 | CE | MET | A | 539 | −43.838 | −60.173 | 97.979 | 1.00 | 208.32 | C |
| ATOM | 3686 | N | GLU | A | 540 | −42.709 | −58.446 | 102.780 | 1.00 | 206.76 | N |
| ATOM | 3687 | CA | GLU | A | 540 | −41.251 | −58.346 | 102.902 | 1.00 | 206.60 | C |
| ATOM | 3688 | C | GLU | A | 540 | −40.880 | −58.410 | 104.379 | 1.00 | 206.07 | C |
| ATOM | 3689 | O | GLU | A | 540 | −39.784 | −58.839 | 104.750 | 1.00 | 206.07 | O |
| ATOM | 3690 | CB | GLU | A | 540 | −40.718 | −57.063 | 102.232 | 1.00 | 206.84 | C |
| ATOM | 3691 | CG | GLU | A | 540 | −40.887 | −55.757 | 103.037 | 1.00 | 207.73 | C |
| ATOM | 3692 | CD | GLU | A | 540 | −42.349 | −55.299 | 103.227 | 1.00 | 208.93 | C |
| ATOM | 3693 | OE1 | GLU | A | 540 | −43.284 | −56.141 | 103.311 | 1.00 | 209.36 | O |
| ATOM | 3694 | OE2 | GLU | A | 540 | −42.555 | −54.068 | 103.310 | 1.00 | 209.22 | O |
| ATOM | 3695 | N | ARG | A | 541 | −41.819 | −57.982 | 105.216 | 1.00 | 205.35 | N |
| ATOM | 3696 | CA | ARG | A | 541 | −41.765 | −58.250 | 106.633 | 1.00 | 204.75 | C |
| ATOM | 3697 | C | ARG | A | 541 | −41.655 | −59.788 | 106.757 | 1.00 | 204.24 | C |
| ATOM | 3698 | O | ARG | A | 541 | −41.484 | −60.345 | 107.851 | 1.00 | 204.43 | O |
| ATOM | 3699 | CB | ARG | A | 541 | −43.007 | −57.644 | 107.296 | 1.00 | 204.77 | C |
| ATOM | 3700 | CG | ARG | A | 541 | −43.114 | −57.797 | 108.793 | 1.00 | 205.32 | C |
| ATOM | 3701 | CD | ARG | A | 541 | −43.674 | −56.558 | 109.436 | 1.00 | 206.22 | C |
| ATOM | 3702 | NE | ARG | A | 541 | −42.611 | −55.570 | 109.611 | 1.00 | 208.12 | N |
| ATOM | 3703 | CZ | ARG | A | 541 | −41.931 | −55.375 | 110.741 | 1.00 | 209.09 | C |
| ATOM | 3704 | NH1 | ARG | A | 541 | −42.206 | −56.089 | 111.830 | 1.00 | 209.37 | N |
| ATOM | 3705 | NH2 | ARG | A | 541 | −40.976 | −54.449 | 110.787 | 1.00 | 209.42 | N |
| ATOM | 3706 | N | ASP | A | 542 | −41.734 | −60.449 | 105.599 | 1.00 | 203.28 | N |
| ATOM | 3707 | CA | ASP | A | 542 | −41.325 | −61.836 | 105.420 | 1.00 | 202.39 | C |
| ATOM | 3708 | C | ASP | A | 542 | −40.032 | −61.908 | 104.615 | 1.00 | 201.99 | C |
| ATOM | 3709 | O | ASP | A | 542 | −38.945 | −62.047 | 105.177 | 1.00 | 201.70 | O |
| ATOM | 3710 | CB | ASP | A | 542 | −42.409 | −62.611 | 104.684 | 1.00 | 202.26 | C |
| ATOM | 3711 | CG | ASP | A | 542 | −43.174 | −63.537 | 105.582 | 1.00 | 201.56 | C |
| ATOM | 3712 | OD1 | ASP | A | 542 | −43.299 | −63.242 | 106.785 | 1.00 | 200.88 | O |
| ATOM | 3713 | OD2 | ASP | A | 542 | −43.646 | −64.572 | 105.074 | 1.00 | 200.81 | O |
| ATOM | 3714 | N | LEU | A | 543 | −40.177 | −61.804 | 103.292 | 1.00 | 201.69 | N |
| ATOM | 3715 | CA | LEU | A | 543 | −39.062 | −61.838 | 102.333 | 1.00 | 201.33 | C |
| ATOM | 3716 | C | LEU | A | 543 | −37.762 | −61.284 | 102.917 | 1.00 | 201.16 | C |
| ATOM | 3717 | O | LEU | A | 543 | −36.779 | −62.018 | 103.033 | 1.00 | 201.31 | O |
| ATOM | 3718 | CB | LEU | A | 543 | −39.429 | −61.099 | 101.026 | 1.00 | 201.22 | C |
| ATOM | 3719 | CG | LEU | A | 543 | −38.293 | −60.544 | 100.153 | 1.00 | 200.47 | C |
| ATOM | 3720 | CD1 | LEU | A | 543 | −37.910 | −61.526 | 99.088 | 1.00 | 199.99 | C |
| ATOM | 3721 | CD2 | LEU | A | 543 | −38.684 | −59.225 | 99.527 | 1.00 | 200.29 | C |
| ATOM | 3722 | N | ALA | A | 544 | −37.771 | −60.004 | 103.302 | 1.00 | 200.71 | N |
| ATOM | 3723 | CA | ALA | A | 544 | −36.564 | −59.322 | 103.772 | 1.00 | 200.07 | C |
| ATOM | 3724 | C | ALA | A | 544 | −35.788 | −60.161 | 104.789 | 1.00 | 199.54 | C |
| ATOM | 3725 | O | ALA | A | 544 | −34.567 | −60.063 | 104.871 | 1.00 | 199.79 | O |
| ATOM | 3726 | CB | ALA | A | 544 | −36.895 | −57.933 | 104.332 | 1.00 | 200.06 | C |
| ATOM | 3727 | N | SER | A | 545 | −36.490 | −61.020 | 105.522 | 1.00 | 198.53 | N |
| ATOM | 3728 | CA | SER | A | 545 | −35.849 | −61.838 | 106.533 | 1.00 | 197.60 | C |
| ATOM | 3729 | C | SER | A | 545 | −35.263 | −63.144 | 105.999 | 1.00 | 197.04 | C |
| ATOM | 3730 | O | SER | A | 545 | −34.663 | −63.897 | 106.755 | 1.00 | 196.80 | O |
| ATOM | 3731 | CB | SER | A | 545 | −36.823 | −62.092 | 107.659 | 1.00 | 197.65 | C |
| ATOM | 3732 | OG | SER | A | 545 | −37.656 | −60.959 | 107.799 | 1.00 | 197.61 | O |
| ATOM | 3733 | N | GLY | A | 546 | −35.428 | −63.403 | 104.702 | 1.00 | 196.61 | N |
| ATOM | 3734 | CA | GLY | A | 546 | −34.719 | −64.508 | 104.024 | 1.00 | 196.22 | C |
| ATOM | 3735 | C | GLY | A | 546 | −35.483 | −65.436 | 103.075 | 1.00 | 195.79 | C |
| ATOM | 3736 | O | GLY | A | 546 | −34.911 | −66.365 | 102.482 | 1.00 | 195.48 | O |
| ATOM | 3737 | N | LEU | A | 547 | −36.774 | −65.176 | 102.915 | 1.00 | 195.56 | N |
| ATOM | 3738 | CA | LEU | A | 547 | −37.664 | −66.080 | 102.186 | 1.00 | 195.32 | C |
| ATOM | 3739 | C | LEU | A | 547 | −37.606 | −65.925 | 100.657 | 1.00 | 194.90 | C |
| ATOM | 3740 | O | LEU | A | 547 | −38.564 | −65.448 | 100.020 | 1.00 | 194.92 | O |
| ATOM | 3741 | CB | LEU | A | 547 | −39.101 | −65.939 | 102.711 | 1.00 | 195.47 | C |
| ATOM | 3742 | CG | LEU | A | 547 | −39.237 | −66.063 | 104.233 | 1.00 | 195.62 | C |
| ATOM | 3743 | CD1 | LEU | A | 547 | −40.567 | −65.503 | 104.673 | 1.00 | 195.63 | C |
| ATOM | 3744 | CD2 | LEU | A | 547 | −39.033 | −67.497 | 104.744 | 1.00 | 195.51 | C |
| ATOM | 3745 | N | ILE | A | 548 | −36.484 | −66.350 | 100.077 | 1.00 | 194.08 | N |
| ATOM | 3746 | CA | ILE | A | 548 | −36.280 | −66.233 | 98.641 | 1.00 | 193.09 | C |
| ATOM | 3747 | C | ILE | A | 548 | −35.686 | −67.503 | 98.024 | 1.00 | 192.88 | C |
| ATOM | 3748 | O | ILE | A | 548 | −34.841 | −68.167 | 98.632 | 1.00 | 192.57 | O |
| ATOM | 3749 | CB | ILE | A | 548 | −35.434 | −64.985 | 98.304 | 1.00 | 192.81 | C |
| ATOM | 3750 | CG1 | ILE | A | 548 | −35.506 | −64.663 | 96.819 | 1.00 | 192.06 | C |
| ATOM | 3751 | CG2 | ILE | A | 548 | −34.005 | −65.157 | 98.763 | 1.00 | 192.87 | C |
| ATOM | 3752 | CD1 | ILE | A | 548 | −36.900 | −64.408 | 96.342 | 1.00 | 191.84 | C |
| ATOM | 3753 | N | GLY | A | 549 | −36.165 | −67.825 | 96.819 | 1.00 | 192.73 | N |
| ATOM | 3754 | CA | GLY | A | 549 | −35.701 | −68.976 | 96.021 | 1.00 | 192.27 | C |
| ATOM | 3755 | C | GLY | A | 549 | −36.144 | −68.937 | 94.554 | 1.00 | 191.81 | C |
| ATOM | 3756 | O | GLY | A | 549 | −37.291 | −68.569 | 94.260 | 1.00 | 191.55 | O |
| ATOM | 3757 | N | PRO | A | 550 | −35.241 | −69.342 | 93.628 | 1.00 | 191.49 | N |
| ATOM | 3758 | CA | PRO | A | 550 | −35.427 | −69.157 | 92.188 | 1.00 | 191.25 | C |
| ATOM | 3759 | C | PRO | A | 550 | −36.267 | −70.245 | 91.497 | 1.00 | 191.03 | C |
| ATOM | 3760 | O | PRO | A | 550 | −35.950 | −71.449 | 91.583 | 1.00 | 190.93 | O |
| ATOM | 3761 | CB | PRO | A | 550 | −33.988 | −69.153 | 91.656 | 1.00 | 191.17 | C |
| ATOM | 3762 | CG | PRO | A | 550 | −33.252 | −70.052 | 92.579 | 1.00 | 191.26 | C |
| ATOM | 3763 | CD | PRO | A | 550 | −33.967 | −70.027 | 93.917 | 1.00 | 191.45 | C |
| ATOM | 3764 | N | LEU | A | 551 | −37.325 | −69.799 | 90.816 | 1.00 | 190.64 | N |
| ATOM | 3765 | CA | LEU | A | 551 | −38.216 | −70.678 | 90.084 | 1.00 | 190.41 | C |
| ATOM | 3766 | C | LEU | A | 551 | −37.979 | −70.501 | 88.609 | 1.00 | 190.96 | C |
| ATOM | 3767 | O | LEU | A | 551 | −37.928 | −69.385 | 88.109 | 1.00 | 190.93 | O |
| ATOM | 3768 | CB | LEU | A | 551 | −39.661 | −70.363 | 90.426 | 1.00 | 189.94 | C |
| ATOM | 3769 | CG | LEU | A | 551 | −40.771 | −71.360 | 90.087 | 1.00 | 188.89 | C |
| ATOM | 3770 | CD1 | LEU | A | 551 | −41.635 | −70.768 | 89.030 | 1.00 | 188.65 | C |
| ATOM | 3771 | CD2 | LEU | A | 551 | −40.301 | −72.766 | 89.697 | 1.00 | 187.48 | C |
| ATOM | 3772 | N | LEU | A | 552 | −37.829 | −71.617 | 87.915 | 1.00 | 191.81 | N |
| ATOM | 3773 | CA | LEU | A | 552 | −37.459 | −71.595 | 86.518 | 1.00 | 192.70 | C |
| ATOM | 3774 | C | LEU | A | 552 | −38.546 | −72.295 | 85.723 | 1.00 | 193.88 | C |
| ATOM | 3775 | O | LEU | A | 552 | −38.782 | −73.487 | 85.924 | 1.00 | 194.05 | O |
| ATOM | 3776 | CB | LEU | A | 552 | −36.122 | −72.318 | 86.338 | 1.00 | 192.12 | C |
| ATOM | 3777 | CG | LEU | A | 552 | −35.084 | −71.704 | 85.407 | 1.00 | 191.07 | C |
| ATOM | 3778 | CD1 | LEU | A | 552 | −34.260 | −70.648 | 86.107 | 1.00 | 189.44 | C |
| ATOM | 3779 | CD2 | LEU | A | 552 | −34.191 | −72.795 | 84.908 | 1.00 | 190.55 | C |
| ATOM | 3780 | N | ILE | A | 553 | −39.217 | −71.553 | 84.842 | 1.00 | 195.30 | N |
| ATOM | 3781 | CA | ILE | A | 553 | −40.192 | −72.148 | 83.926 | 1.00 | 196.99 | C |
| ATOM | 3782 | C | ILE | A | 553 | −39.626 | −72.227 | 82.498 | 1.00 | 198.56 | C |
| ATOM | 3783 | O | ILE | A | 553 | −39.067 | −71.241 | 81.993 | 1.00 | 198.73 | O |
| ATOM | 3784 | CB | ILE | A | 553 | −41.508 | −71.378 | 83.919 | 1.00 | 196.69 | C |
| ATOM | 3785 | CG1 | ILE | A | 553 | −41.906 | −70.984 | 85.336 | 1.00 | 196.84 | C |
| ATOM | 3786 | CG2 | ILE | A | 553 | −42.591 | −72.217 | 83.298 | 1.00 | 196.60 | C |
| ATOM | 3787 | CD1 | ILE | A | 553 | −43.226 | −70.204 | 85.434 | 1.00 | 197.09 | C |
| ATOM | 3788 | N | CYS | A | 554 | −39.779 | −73.392 | 81.851 | 1.00 | 200.46 | N |
| ATOM | 3789 | CA | CYS | A | 554 | −39.080 | −73.680 | 80.590 | 1.00 | 202.37 | C |
| ATOM | 3790 | C | CYS | A | 554 | −39.832 | −74.443 | 79.507 | 1.00 | 204.39 | C |
| ATOM | 3791 | O | CYS | A | 554 | −40.743 | −75.216 | 79.782 | 1.00 | 204.49 | O |
| ATOM | 3792 | CB | CYS | A | 554 | −37.780 | −74.418 | 80.881 | 1.00 | 201.93 | C |
| ATOM | 3793 | SG | CYS | A | 554 | −36.631 | −73.458 | 81.872 | 1.00 | 201.16 | S |
| ATOM | 3794 | N | TYR | A | 555 | −39.392 | −74.207 | 78.273 | 1.00 | 207.19 | N |
| ATOM | 3795 | CA | TYR | A | 555 | −39.762 | −74.925 | 77.048 | 1.00 | 210.07 | C |
| ATOM | 3796 | C | TYR | A | 555 | −39.631 | −76.452 | 77.160 | 1.00 | 211.83 | C |
| ATOM | 3797 | O | TYR | A | 555 | −38.891 | −76.960 | 78.003 | 1.00 | 212.00 | O |
| ATOM | 3798 | CB | TYR | A | 555 | −38.813 | −74.427 | 75.964 | 1.00 | 210.46 | C |
| ATOM | 3799 | CG | TYR | A | 555 | −39.236 | −74.621 | 74.536 | 1.00 | 211.55 | C |
| ATOM | 3800 | CD1 | TYR | A | 555 | −40.102 | −73.712 | 73.913 | 1.00 | 212.42 | C |
| ATOM | 3801 | CD2 | TYR | A | 555 | −38.721 | −75.679 | 73.778 | 1.00 | 212.37 | C |
| ATOM | 3802 | CE1 | TYR | A | 555 | −40.477 | −73.874 | 72.573 | 1.00 | 212.77 | C |
| ATOM | 3803 | CE2 | TYR | A | 555 | −39.084 | −75.852 | 72.437 | 1.00 | 212.71 | C |
| ATOM | 3804 | CZ | TYR | A | 555 | −39.962 | −74.948 | 71.842 | 1.00 | 212.37 | C |
| ATOM | 3805 | OH | TYR | A | 555 | −40.321 | −75.120 | 70.522 | 1.00 | 211.75 | O |
| ATOM | 3806 | N | LYS | A | 556 | −40.334 | −77.178 | 76.296 | 1.00 | 214.04 | N |
| ATOM | 3807 | CA | LYS | A | 556 | −40.355 | −78.638 | 76.350 | 1.00 | 216.86 | C |
| ATOM | 3808 | C | LYS | A | 556 | −39.085 | −79.260 | 75.741 | 1.00 | 217.74 | C |
| ATOM | 3809 | O | LYS | A | 556 | −38.603 | −78.755 | 74.728 | 1.00 | 218.02 | O |
| ATOM | 3810 | CB | LYS | A | 556 | −41.611 | −79.135 | 75.630 | 1.00 | 216.13 | C |
| ATOM | 3811 | CG | LYS | A | 556 | −41.974 | −80.582 | 75.897 | 1.00 | 216.10 | C |
| ATOM | 3812 | CD | LYS | A | 556 | −42.105 | −80.872 | 77.381 | 1.00 | 214.92 | C |
| ATOM | 3813 | CE | LYS | A | 556 | −42.288 | −82.352 | 77.622 | 1.00 | 214.33 | C |
| ATOM | 3814 | NZ | LYS | A | 556 | −42.033 | −82.670 | 79.041 | 1.00 | 213.83 | N |
| ATOM | 3815 | N | GLU | A | 557 | −38.545 | −80.326 | 76.364 | 1.00 | 219.53 | N |
| ATOM | 3816 | CA | GLU | A | 557 | −37.347 | −81.095 | 75.858 | 1.00 | 221.09 | C |
| ATOM | 3817 | C | GLU | A | 557 | −36.826 | −82.270 | 76.742 | 1.00 | 222.25 | C |
| ATOM | 3818 | O | GLU | A | 557 | −36.703 | −82.109 | 77.961 | 1.00 | 222.53 | O |
| ATOM | 3819 | CB | GLU | A | 557 | −36.166 | −80.156 | 75.512 | 1.00 | 221.26 | C |
| ATOM | 3820 | CG | GLU | A | 557 | −35.637 | −79.283 | 76.668 | 1.00 | 221.96 | C |
| ATOM | 3821 | CD | GLU | A | 557 | −35.150 | −77.909 | 76.209 | 1.00 | 222.89 | C |
| ATOM | 3822 | OE1 | GLU | A | 557 | −35.103 | −77.663 | 74.978 | 1.00 | 223.14 | O |
| ATOM | 3823 | OE2 | GLU | A | 557 | −34.819 | −77.073 | 77.087 | 1.00 | 223.04 | O |
| ATOM | 3824 | N | SER | A | 558 | −36.521 | −83.427 | 76.120 | 1.00 | 223.64 | N |
| ATOM | 3825 | CA | SER | A | 558 | −35.807 | −84.591 | 76.768 | 1.00 | 224.53 | C |
| ATOM | 3826 | C | SER | A | 558 | −35.316 | −85.676 | 75.768 | 1.00 | 226.42 | C |
| ATOM | 3827 | O | SER | A | 558 | −36.128 | −86.460 | 75.243 | 1.00 | 226.55 | O |
| ATOM | 3828 | CB | SER | A | 558 | −36.627 | −85.241 | 77.911 | 1.00 | 224.77 | C |
| ATOM | 3829 | OG | SER | A | 558 | −37.265 | −86.456 | 77.531 | 1.00 | 224.24 | O |
| ATOM | 3830 | N | VAL | A | 559 | −33.991 | −85.731 | 75.549 | 1.00 | 228.24 | N |
| ATOM | 3831 | CA | VAL | A | 559 | −33.350 | −86.477 | 74.420 | 1.00 | 230.02 | C |
| ATOM | 3832 | C | VAL | A | 559 | −34.165 | −86.330 | 73.100 | 1.00 | 231.36 | C |
| ATOM | 3833 | O | VAL | A | 559 | −34.513 | −87.327 | 72.433 | 1.00 | 231.58 | O |
| ATOM | 3834 | CB | VAL | A | 559 | −32.977 | −87.968 | 74.771 | 1.00 | 229.90 | C |
| ATOM | 3835 | CG1 | VAL | A | 559 | −31.954 | −88.524 | 73.774 | 1.00 | 229.78 | C |
| ATOM | 3836 | CG2 | VAL | A | 559 | −32.409 | −88.069 | 76.183 | 1.00 | 229.93 | C |
| ATOM | 3837 | N | ASP | A | 560 | −34.448 | −85.057 | 72.768 | 1.00 | 232.89 | N |
| ATOM | 3838 | CA | ASP | A | 560 | −35.320 | −84.587 | 71.657 | 1.00 | 234.25 | C |
| ATOM | 3839 | C | ASP | A | 560 | −35.536 | −83.047 | 71.804 | 1.00 | 234.71 | C |
| ATOM | 3840 | O | ASP | A | 560 | −35.215 | −82.469 | 72.861 | 1.00 | 234.88 | O |
| ATOM | 3841 | CB | ASP | A | 560 | −36.674 | −85.336 | 71.650 | 1.00 | 234.05 | C |
| ATOM | 3842 | CG | ASP | A | 560 | −37.257 | −85.521 | 70.244 | 1.00 | 233.88 | C |
| ATOM | 3843 | OD1 | ASP | A | 560 | −38.445 | −85.949 | 70.176 | 1.00 | 233.48 | O |
| ATOM | 3844 | OD2 | ASP | A | 560 | −36.544 | −85.249 | 69.221 | 1.00 | 233.30 | O |
| ATOM | 3845 | N | GLN | A | 561 | −36.068 | −82.402 | 70.753 | 1.00 | 235.32 | N |
| ATOM | 3846 | CA | GLN | A | 561 | −36.428 | −80.947 | 70.731 | 1.00 | 235.58 | C |
| ATOM | 3847 | C | GLN | A | 561 | −35.301 | −80.011 | 70.215 | 1.00 | 236.18 | C |
| ATOM | 3848 | O | GLN | A | 561 | −35.239 | −79.744 | 69.010 | 1.00 | 236.21 | O |
| ATOM | 3849 | CB | GLN | A | 561 | −37.047 | −80.460 | 72.065 | 1.00 | 235.73 | C |
| ATOM | 3850 | CG | GLN | A | 561 | −38.285 | −81.261 | 72.546 | 1.00 | 235.51 | C |
| ATOM | 3851 | CD | GLN | A | 561 | −39.614 | −80.545 | 72.337 | 1.00 | 235.49 | C |
| ATOM | 3852 | OE1 | GLN | A | 561 | −39.658 | −79.362 | 72.001 | 1.00 | 235.68 | O |
| ATOM | 3853 | NE2 | GLN | A | 561 | −40.709 | −81.266 | 72.556 | 1.00 | 235.37 | N |
| ATOM | 3854 | N | ARG | A | 562 | −34.439 | −79.509 | 71.109 | 1.00 | 236.70 | N |
| ATOM | 3855 | CA | ARG | A | 562 | −33.203 | −78.806 | 70.700 | 1.00 | 237.26 | C |
| ATOM | 3856 | C | ARG | A | 562 | −32.092 | −78.751 | 71.752 | 1.00 | 237.53 | C |
| ATOM | 3857 | O | ARG | A | 562 | −32.327 | −78.367 | 72.903 | 1.00 | 237.49 | O |
| ATOM | 3858 | CB | ARG | A | 562 | −33.487 | −77.408 | 70.135 | 1.00 | 237.33 | C |
| ATOM | 3859 | CG | ARG | A | 562 | −33.022 | −77.250 | 68.700 | 1.00 | 237.80 | C |
| ATOM | 3860 | CD | ARG | A | 562 | −33.679 | −76.078 | 68.004 | 1.00 | 238.74 | C |
| ATOM | 3861 | NE | ARG | A | 562 | −33.200 | −75.952 | 66.628 | 1.00 | 239.61 | N |
| ATOM | 3862 | CZ | ARG | A | 562 | −33.820 | −75.277 | 65.661 | 1.00 | 240.09 | C |
| ATOM | 3863 | NH1 | ARG | A | 562 | −34.969 | −74.655 | 65.903 | 1.00 | 240.32 | N |
| ATOM | 3864 | NH2 | ARG | A | 562 | −33.289 | −75.229 | 64.442 | 1.00 | 240.17 | N |
| ATOM | 3865 | N | GLY | A | 563 | −30.886 | −79.138 | 71.325 | 1.00 | 237.89 | N |
| ATOM | 3866 | CA | GLY | A | 563 | −29.680 | −79.125 | 72.159 | 1.00 | 238.31 | C |
| ATOM | 3867 | C | GLY | A | 563 | −29.662 | −80.149 | 73.282 | 1.00 | 238.58 | C |
| ATOM | 3868 | O | GLY | A | 563 | −29.568 | −79.781 | 74.460 | 1.00 | 238.72 | O |
| ATOM | 3869 | N | ASN | A | 564 | −29.751 | −81.430 | 72.912 | 1.00 | 238.72 | N |
| ATOM | 3870 | CA | ASN | A | 564 | −29.762 | −82.555 | 73.866 | 1.00 | 238.81 | C |
| ATOM | 3871 | C | ASN | A | 564 | −29.065 | −83.797 | 73.276 | 1.00 | 238.84 | C |
| ATOM | 3872 | O | ASN | A | 564 | −29.658 | −84.886 | 73.215 | 1.00 | 238.81 | O |
| ATOM | 3873 | CB | ASN | A | 564 | −31.207 | −82.898 | 74.315 | 1.00 | 238.82 | C |
| ATOM | 3874 | CG | ASN | A | 564 | −31.602 | −82.256 | 75.662 | 1.00 | 238.65 | C |
| ATOM | 3875 | OD1 | ASN | A | 564 | −31.061 | −82.601 | 76.717 | 1.00 | 238.45 | O |
| ATOM | 3876 | ND2 | ASN | A | 564 | −32.581 | −81.352 | 75.623 | 1.00 | 238.05 | N |
| ATOM | 3877 | N | GLN | A | 565 | −27.808 | −83.625 | 72.851 | 1.00 | 238.90 | N |
| ATOM | 3878 | CA | GLN | A | 565 | −27.041 | −84.709 | 72.198 | 1.00 | 238.98 | C |
| ATOM | 3879 | C | GLN | A | 565 | −25.731 | −85.148 | 72.902 | 1.00 | 239.10 | C |
| ATOM | 3880 | O | GLN | A | 565 | −25.327 | −86.307 | 72.752 | 1.00 | 239.20 | O |
| ATOM | 3881 | CB | GLN | A | 565 | −26.782 | −84.389 | 70.714 | 1.00 | 238.92 | C |
| ATOM | 3882 | CG | GLN | A | 565 | −26.697 | −85.619 | 69.789 | 1.00 | 238.54 | C |
| ATOM | 3883 | CD | GLN | A | 565 | −25.337 | −86.325 | 69.800 | 1.00 | 238.24 | C |
| ATOM | 3884 | OE1 | GLN | A | 565 | −24.294 | −85.713 | 70.046 | 1.00 | 238.05 | O |
| ATOM | 3885 | NE2 | GLN | A | 565 | −25.351 | −87.622 | 69.521 | 1.00 | 238.16 | N |
| ATOM | 3886 | N | ILE | A | 566 | −25.065 | −84.243 | 73.637 | 1.00 | 239.13 | N |
| ATOM | 3887 | CA | ILE | A | 566 | −23.883 | −84.605 | 74.481 | 1.00 | 238.97 | C |
| ATOM | 3888 | C | ILE | A | 566 | −23.964 | −84.113 | 75.968 | 1.00 | 239.01 | C |
| ATOM | 3889 | O | ILE | A | 566 | −23.523 | −84.834 | 76.875 | 1.00 | 239.03 | O |
| ATOM | 3890 | CB | ILE | A | 566 | −22.479 | −84.319 | 73.772 | 1.00 | 239.02 | C |
| ATOM | 3891 | CG1 | ILE | A | 566 | −22.218 | −85.315 | 72.620 | 1.00 | 238.82 | C |
| ATOM | 3892 | CG2 | ILE | A | 566 | −21.311 | −84.358 | 74.775 | 1.00 | 239.05 | C |
| ATOM | 3893 | CD1 | ILE | A | 566 | −21.028 | −84.987 | 71.709 | 1.00 | 238.66 | C |
| ATOM | 3894 | N | MET | A | 567 | −24.524 | −82.910 | 76.193 | 1.00 | 238.87 | N |
| ATOM | 3895 | CA | MET | A | 567 | −24.914 | −82.372 | 77.538 | 1.00 | 238.69 | C |
| ATOM | 3896 | C | MET | A | 567 | −25.568 | −80.979 | 77.473 | 1.00 | 238.15 | C |
| ATOM | 3897 | O | MET | A | 567 | −25.205 | −80.158 | 76.627 | 1.00 | 238.12 | O |
| ATOM | 3898 | CB | MET | A | 567 | −23.730 | −82.318 | 78.532 | 1.00 | 238.85 | C |
| ATOM | 3899 | CG | MET | A | 567 | −23.857 | −83.230 | 79.787 | 1.00 | 239.51 | C |
| ATOM | 3900 | SD | MET | A | 567 | −24.731 | −82.564 | 81.248 | 1.00 | 240.49 | S |
| ATOM | 3901 | CE | MET | A | 567 | −23.614 | −81.269 | 81.805 | 1.00 | 240.09 | C |
| ATOM | 3902 | N | SER | A | 568 | −26.525 | −80.719 | 78.368 | 1.00 | 237.57 | N |
| ATOM | 3903 | CA | SER | A | 568 | −26.999 | −79.345 | 78.609 | 1.00 | 236.99 | C |
| ATOM | 3904 | C | SER | A | 568 | −26.355 | −78.755 | 79.890 | 1.00 | 236.47 | C |
| ATOM | 3905 | O | SER | A | 568 | −26.834 | −78.991 | 81.015 | 1.00 | 236.51 | O |
| ATOM | 3906 | CB | SER | A | 568 | −28.542 | −79.246 | 78.611 | 1.00 | 237.03 | C |
| ATOM | 3907 | OG | SER | A | 568 | −29.118 | −79.661 | 79.840 | 1.00 | 237.01 | O |
| ATOM | 3908 | N | ASP | A | 569 | −25.260 | −78.002 | 79.682 | 1.00 | 235.61 | N |
| ATOM | 3909 | CA | ASP | A | 569 | −24.389 | −77.415 | 80.742 | 1.00 | 234.46 | C |
| ATOM | 3910 | C | ASP | A | 569 | −25.174 | −76.615 | 81.826 | 1.00 | 233.35 | C |
| ATOM | 3911 | O | ASP | A | 569 | −26.008 | −75.761 | 81.489 | 1.00 | 233.36 | O |
| ATOM | 3912 | CB | ASP | A | 569 | −23.255 | −76.545 | 80.114 | 1.00 | 234.68 | C |
| ATOM | 3913 | CG | ASP | A | 569 | −22.288 | −77.344 | 79.178 | 1.00 | 234.63 | C |
| ATOM | 3914 | OD1 | ASP | A | 569 | −21.772 | −78.415 | 79.577 | 1.00 | 234.70 | O |
| ATOM | 3915 | OD2 | ASP | A | 569 | −22.010 | −76.866 | 78.048 | 1.00 | 233.96 | O |
| ATOM | 3916 | N | LYS | A | 570 | −24.863 | −76.883 | 83.106 | 1.00 | 231.71 | N |
| ATOM | 3917 | CA | LYS | A | 570 | −25.709 | −76.538 | 84.294 | 1.00 | 229.88 | C |
| ATOM | 3918 | C | LYS | A | 570 | −26.360 | −75.141 | 84.419 | 1.00 | 228.61 | C |
| ATOM | 3919 | O | LYS | A | 570 | −26.067 | −74.202 | 83.658 | 1.00 | 228.53 | O |
| ATOM | 3920 | CB | LYS | A | 570 | −24.986 | −76.884 | 85.625 | 1.00 | 229.87 | C |
| ATOM | 3921 | CG | LYS | A | 570 | −24.614 | −78.361 | 85.823 | 1.00 | 229.29 | C |
| ATOM | 3922 | CD | LYS | A | 570 | −25.799 | −79.281 | 85.628 | 1.00 | 228.02 | C |
| ATOM | 3923 | CE | LYS | A | 570 | −25.407 | −80.455 | 84.781 | 1.00 | 227.27 | C |
| ATOM | 3924 | NZ | LYS | A | 570 | −26.405 | −80.624 | 83.702 | 1.00 | 226.80 | N |
| ATOM | 3925 | N | ARG | A | 571 | −27.236 | −75.032 | 85.421 | 1.00 | 226.77 | N |
| ATOM | 3926 | CA | ARG | A | 571 | −28.006 | −73.818 | 85.699 | 1.00 | 224.85 | C |
| ATOM | 3927 | C | ARG | A | 571 | −27.433 | −73.055 | 86.914 | 1.00 | 222.81 | C |
| ATOM | 3928 | O | ARG | A | 571 | −27.016 | −73.672 | 87.914 | 1.00 | 222.46 | O |
| ATOM | 3929 | CB | ARG | A | 571 | −29.502 | −74.140 | 85.899 | 1.00 | 225.35 | C |
| ATOM | 3930 | CG | ARG | A | 571 | −30.008 | −75.478 | 85.312 | 1.00 | 226.93 | C |
| ATOM | 3931 | CD | ARG | A | 571 | −30.365 | −76.485 | 86.437 | 1.00 | 230.67 | C |
| ATOM | 3932 | NE | ARG | A | 571 | −29.302 | −77.449 | 86.792 | 1.00 | 233.69 | N |
| ATOM | 3933 | CZ | ARG | A | 571 | −28.401 | −77.307 | 87.777 | 1.00 | 234.84 | C |
| ATOM | 3934 | NH1 | ARG | A | 571 | −28.377 | −76.215 | 88.540 | 1.00 | 235.33 | N |
| ATOM | 3935 | NH2 | ARG | A | 571 | −27.503 | −78.267 | 87.997 | 1.00 | 234.98 | N |
| ATOM | 3936 | N | ASN | A | 572 | −27.435 | −71.719 | 86.806 | 1.00 | 220.19 | N |
| ATOM | 3937 | CA | ASN | A | 572 | −26.714 | −70.815 | 87.720 | 1.00 | 217.53 | C |
| ATOM | 3938 | C | ASN | A | 572 | −27.408 | −69.437 | 87.893 | 1.00 | 215.36 | C |
| ATOM | 3939 | O | ASN | A | 572 | −27.671 | −68.748 | 86.913 | 1.00 | 214.96 | O |
| ATOM | 3940 | CB | ASN | A | 572 | −25.281 | −70.645 | 87.197 | 1.00 | 217.69 | C |
| ATOM | 3941 | CG | ASN | A | 572 | −24.217 | −70.734 | 88.298 | 1.00 | 218.12 | C |
| ATOM | 3942 | OD1 | ASN | A | 572 | −24.444 | −70.349 | 89.449 | 1.00 | 218.30 | O |
| ATOM | 3943 | ND2 | ASN | A | 572 | −23.034 | −71.231 | 87.929 | 1.00 | 218.62 | N |
| ATOM | 3944 | N | VAL | A | 573 | −27.689 | −69.043 | 89.137 | 1.00 | 212.83 | N |
| ATOM | 3945 | CA | VAL | A | 573 | −28.538 | −67.871 | 89.431 | 1.00 | 210.38 | C |
| ATOM | 3946 | C | VAL | A | 573 | −27.908 | −66.758 | 90.248 | 1.00 | 208.61 | C |
| ATOM | 3947 | O | VAL | A | 573 | −27.354 | −66.992 | 91.318 | 1.00 | 208.23 | O |
| ATOM | 3948 | CB | VAL | A | 573 | −29.817 | −68.258 | 90.202 | 1.00 | 210.53 | C |
| ATOM | 3949 | CG1 | VAL | A | 573 | −30.961 | −68.514 | 89.252 | 1.00 | 210.63 | C |
| ATOM | 3950 | CG2 | VAL | A | 573 | −29.566 | −69.451 | 91.133 | 1.00 | 210.62 | C |
| ATOM | 3951 | N | ILE | A | 574 | −28.042 | −65.539 | 89.745 | 1.00 | 206.58 | N |
| ATOM | 3952 | CA | ILE | A | 574 | −27.658 | −64.348 | 90.478 | 1.00 | 204.79 | C |
| ATOM | 3953 | C | ILE | A | 574 | −28.947 | −63.651 | 90.917 | 1.00 | 203.90 | C |
| ATOM | 3954 | O | ILE | A | 574 | −29.891 | −63.543 | 90.141 | 1.00 | 203.61 | O |
| ATOM | 3955 | CB | ILE | A | 574 | −26.749 | −63.399 | 89.623 | 1.00 | 204.68 | C |
| ATOM | 3956 | CG1 | ILE | A | 574 | −25.449 | −64.089 | 89.201 | 1.00 | 204.48 | C |
| ATOM | 3957 | CG2 | ILE | A | 574 | −26.385 | −62.126 | 90.365 | 1.00 | 204.14 | C |
| ATOM | 3958 | CD1 | ILE | A | 574 | −25.317 | −64.343 | 87.712 | 1.00 | 204.38 | C |
| ATOM | 3959 | N | LEU | A | 575 | −28.988 | −63.209 | 92.172 | 1.00 | 202.88 | N |
| ATOM | 3960 | CA | LEU | A | 575 | −30.111 | −62.442 | 92.705 | 1.00 | 201.81 | C |
| ATOM | 3961 | C | LEU | A | 575 | −29.663 | −61.116 | 93.289 | 1.00 | 201.42 | C |
| ATOM | 3962 | O | LEU | A | 575 | −28.967 | −61.079 | 94.300 | 1.00 | 201.01 | O |
| ATOM | 3963 | CB | LEU | A | 575 | −30.849 | −63.236 | 93.774 | 1.00 | 201.62 | C |
| ATOM | 3964 | CG | LEU | A | 575 | −31.590 | −62.419 | 94.830 | 1.00 | 201.04 | C |
| ATOM | 3965 | CD1 | LEU | A | 575 | −32.582 | −61.461 | 94.199 | 1.00 | 200.63 | C |
| ATOM | 3966 | CD2 | LEU | A | 575 | −32.283 | −63.341 | 95.794 | 1.00 | 200.65 | C |
| ATOM | 3967 | N | PHE | A | 576 | −30.089 | −60.035 | 92.645 | 1.00 | 201.27 | N |
| ATOM | 3968 | CA | PHE | A | 576 | −29.794 | −58.685 | 93.104 | 1.00 | 201.35 | C |
| ATOM | 3969 | C | PHE | A | 576 | −30.800 | −58.282 | 94.166 | 1.00 | 201.49 | C |
| ATOM | 3970 | O | PHE | A | 576 | −31.980 | −58.045 | 93.864 | 1.00 | 201.78 | O |
| ATOM | 3971 | CB | PHE | A | 576 | −29.793 | −57.693 | 91.932 | 1.00 | 201.20 | C |
| ATOM | 3972 | CG | PHE | A | 576 | −28.540 | −57.741 | 91.112 | 1.00 | 201.20 | C |
| ATOM | 3973 | CD1 | PHE | A | 576 | −28.442 | −58.588 | 90.014 | 1.00 | 201.05 | C |
| ATOM | 3974 | CD2 | PHE | A | 576 | −27.439 | −56.960 | 91.451 | 1.00 | 201.47 | C |
| ATOM | 3975 | CE1 | PHE | A | 576 | −27.263 | −58.654 | 89.261 | 1.00 | 201.04 | C |
| ATOM | 3976 | CE2 | PHE | A | 576 | −26.252 | −57.020 | 90.700 | 1.00 | 201.44 | C |
| ATOM | 3977 | CZ | PHE | A | 576 | −26.168 | −57.870 | 89.606 | 1.00 | 201.13 | C |
| ATOM | 3978 | N | SER | A | 577 | −30.333 | −58.213 | 95.414 | 1.00 | 201.42 | N |
| ATOM | 3979 | CA | SER | A | 577 | −31.232 | −57.975 | 96.548 | 1.00 | 201.04 | C |
| ATOM | 3980 | C | SER | A | 577 | −30.715 | −57.099 | 97.692 | 1.00 | 200.56 | C |
| ATOM | 3981 | O | SER | A | 577 | −29.688 | −57.382 | 98.327 | 1.00 | 200.25 | O |
| ATOM | 3982 | CB | SER | A | 577 | −31.755 | −59.301 | 97.108 | 1.00 | 201.17 | C |
| ATOM | 3983 | OG | SER | A | 577 | −32.847 | −59.073 | 97.982 | 1.00 | 201.40 | O |
| ATOM | 3984 | N | VAL | A | 578 | −31.471 | −56.034 | 97.935 | 1.00 | 200.06 | N |
| ATOM | 3985 | CA | VAL | A | 578 | −31.351 | −55.240 | 99.141 | 1.00 | 199.63 | C |
| ATOM | 3986 | C | VAL | A | 578 | −32.288 | −55.837 | 100.179 | 1.00 | 199.30 | C |
| ATOM | 3987 | O | VAL | A | 578 | −33.513 | −55.850 | 100.004 | 1.00 | 199.06 | O |
| ATOM | 3988 | CB | VAL | A | 578 | −31.683 | −53.748 | 98.896 | 1.00 | 199.61 | C |
| ATOM | 3989 | CG1 | VAL | A | 578 | −31.754 | −52.974 | 100.188 | 1.00 | 199.52 | C |
| ATOM | 3990 | CG2 | VAL | A | 578 | −30.645 | −53.133 | 98.014 | 1.00 | 199.93 | C |
| ATOM | 3991 | N | PHE | A | 579 | −31.680 | −56.350 | 101.245 | 1.00 | 198.97 | N |
| ATOM | 3992 | CA | PHE | A | 579 | −32.392 | −56.821 | 102.416 | 1.00 | 198.67 | C |
| ATOM | 3993 | C | PHE | A | 579 | −32.448 | −55.714 | 103.460 | 1.00 | 198.75 | C |
| ATOM | 3994 | O | PHE | A | 579 | −31.405 | −55.216 | 103.903 | 1.00 | 198.75 | O |
| ATOM | 3995 | CB | PHE | A | 579 | −31.684 | −58.037 | 102.985 | 1.00 | 198.42 | C |
| ATOM | 3996 | CG | PHE | A | 579 | −31.714 | −59.215 | 102.084 | 1.00 | 197.92 | C |
| ATOM | 3997 | CD1 | PHE | A | 579 | −32.787 | −60.098 | 102.121 | 1.00 | 197.17 | C |
| ATOM | 3998 | CD2 | PHE | A | 579 | −30.673 | −59.444 | 101.192 | 1.00 | 197.84 | C |
| ATOM | 3999 | CE1 | PHE | A | 579 | −32.824 | −61.193 | 101.292 | 1.00 | 197.14 | C |
| ATOM | 4000 | CE2 | PHE | A | 579 | −30.696 | −60.541 | 100.352 | 1.00 | 197.90 | C |
| ATOM | 4001 | CZ | PHE | A | 579 | −31.776 | −61.420 | 100.401 | 1.00 | 197.84 | C |
| ATOM | 4002 | N | ASP | A | 580 | −33.664 | −55.330 | 103.847 | 1.00 | 198.77 | N |
| ATOM | 4003 | CA | ASP | A | 580 | −33.857 | −54.217 | 104.774 | 1.00 | 198.78 | C |
| ATOM | 4004 | C | ASP | A | 580 | −34.122 | −54.716 | 106.181 | 1.00 | 198.66 | C |
| ATOM | 4005 | O | ASP | A | 580 | −35.265 | −54.695 | 106.654 | 1.00 | 198.58 | O |
| ATOM | 4006 | CB | ASP | A | 580 | −34.995 | −53.305 | 104.309 | 1.00 | 198.90 | C |
| ATOM | 4007 | CG | ASP | A | 580 | −34.870 | −51.890 | 104.856 | 1.00 | 199.17 | C |
| ATOM | 4008 | OD1 | ASP | A | 580 | −33.728 | −51.389 | 104.973 | 1.00 | 198.90 | O |
| ATOM | 4009 | OD2 | ASP | A | 580 | −35.918 | −51.275 | 105.157 | 1.00 | 199.82 | O |
| ATOM | 4010 | N | GLU | A | 581 | −33.051 | −55.148 | 106.843 | 1.00 | 198.53 | N |
| ATOM | 4011 | CA | GLU | A | 581 | −33.129 | −55.720 | 108.177 | 1.00 | 198.53 | C |
| ATOM | 4012 | C | GLU | A | 581 | −34.095 | −54.945 | 109.051 | 1.00 | 198.81 | C |
| ATOM | 4013 | O | GLU | A | 581 | −34.768 | −55.519 | 109.908 | 1.00 | 198.82 | O |
| ATOM | 4014 | CB | GLU | A | 581 | −31.748 | −55.773 | 108.825 | 1.00 | 198.36 | C |
| ATOM | 4015 | CG | GLU | A | 581 | −30.855 | −56.886 | 108.305 | 1.00 | 197.96 | C |
| ATOM | 4016 | CD | GLU | A | 581 | −31.358 | −58.291 | 108.639 | 1.00 | 197.69 | C |
| ATOM | 4017 | OE1 | GLU | A | 581 | −32.123 | −58.466 | 109.618 | 1.00 | 197.57 | O |
| ATOM | 4018 | OE2 | GLU | A | 581 | −30.971 | −59.230 | 107.910 | 1.00 | 197.11 | O |
| ATOM | 4019 | N | ASN | A | 582 | −34.179 | −53.642 | 108.796 | 1.00 | 199.19 | N |
| ATOM | 4020 | CA | ASN | A | 582 | −35.067 | −52.740 | 109.526 | 1.00 | 199.78 | C |
| ATOM | 4021 | C | ASN | A | 582 | −36.539 | −53.149 | 109.538 | 1.00 | 199.99 | C |
| ATOM | 4022 | O | ASN | A | 582 | −37.353 | −52.558 | 110.258 | 1.00 | 200.27 | O |
| ATOM | 4023 | CB | ASN | A | 582 | −34.944 | −51.322 | 108.968 | 1.00 | 199.88 | C |
| ATOM | 4024 | CG | ASN | A | 582 | −33.510 | −50.855 | 108.871 | 1.00 | 200.19 | C |
| ATOM | 4025 | OD1 | ASN | A | 582 | −32.624 | −51.351 | 109.575 | 1.00 | 199.90 | O |
| ATOM | 4026 | ND2 | ASN | A | 582 | −33.271 | −49.890 | 107.990 | 1.00 | 200.93 | N |
| ATOM | 4027 | N | ARG | A | 583 | −36.879 | −54.157 | 108.748 | 1.00 | 200.04 | N |
| ATOM | 4028 | CA | ARG | A | 583 | −38.249 | −54.604 | 108.660 | 1.00 | 200.20 | C |
| ATOM | 4029 | C | ARG | A | 583 | −38.358 | −56.076 | 109.023 | 1.00 | 199.97 | C |
| ATOM | 4030 | O | ARG | A | 583 | −39.465 | −56.593 | 109.182 | 1.00 | 199.91 | O |
| ATOM | 4031 | CB | ARG | A | 583 | −38.763 | −54.377 | 107.241 | 1.00 | 200.42 | C |
| ATOM | 4032 | CG | ARG | A | 583 | −40.200 | −53.878 | 107.153 | 1.00 | 201.79 | C |
| ATOM | 4033 | CD | ARG | A | 583 | −40.251 | −52.354 | 107.087 | 1.00 | 204.00 | C |
| ATOM | 4034 | NE | ARG | A | 583 | −41.152 | −51.854 | 106.038 | 1.00 | 206.00 | N |
| ATOM | 4035 | CZ | ARG | A | 583 | −40.808 | −51.652 | 104.760 | 1.00 | 207.03 | C |
| ATOM | 4036 | NH1 | ARG | A | 583 | −41.705 | −51.186 | 103.894 | 1.00 | 207.21 | N |
| ATOM | 4037 | NH2 | ARG | A | 583 | −39.573 | −51.916 | 104.334 | 1.00 | 207.50 | N |
| ATOM | 4038 | N | SER | A | 584 | −37.205 | −56.738 | 109.167 | 1.00 | 199.82 | N |
| ATOM | 4039 | CA | SER | A | 584 | −37.140 | −58.210 | 109.253 | 1.00 | 199.55 | C |
| ATOM | 4040 | C | SER | A | 584 | −37.823 | −58.771 | 110.505 | 1.00 | 199.27 | C |
| ATOM | 4041 | O | SER | A | 584 | −37.719 | −58.202 | 111.595 | 1.00 | 199.25 | O |
| ATOM | 4042 | CB | SER | A | 584 | −35.696 | −58.756 | 109.059 | 1.00 | 199.57 | C |
| ATOM | 4043 | OG | SER | A | 584 | −35.058 | −59.172 | 110.259 | 1.00 | 199.37 | O |
| ATOM | 4044 | N | TRP | A | 585 | −38.536 | −59.881 | 110.320 | 1.00 | 198.75 | N |
| ATOM | 4045 | CA | TRP | A | 585 | −39.297 | −60.517 | 111.385 | 1.00 | 198.21 | C |
| ATOM | 4046 | C | TRP | A | 585 | −38.415 | −61.041 | 112.533 | 1.00 | 198.06 | C |
| ATOM | 4047 | O | TRP | A | 585 | −38.858 | −61.823 | 113.376 | 1.00 | 198.06 | O |
| ATOM | 4048 | CB | TRP | A | 585 | −40.247 | −61.571 | 110.790 | 1.00 | 198.06 | C |
| ATOM | 4049 | CG | TRP | A | 585 | −41.667 | −61.121 | 110.880 | 1.00 | 197.88 | C |
| ATOM | 4050 | CD1 | TRP | A | 585 | −42.092 | −59.866 | 111.191 | 1.00 | 198.23 | C |
| ATOM | 4051 | CD2 | TRP | A | 585 | −42.854 | −61.903 | 110.676 | 1.00 | 198.00 | C |
| ATOM | 4052 | NE1 | TRP | A | 585 | −43.463 | −59.811 | 111.204 | 1.00 | 198.46 | N |
| ATOM | 4053 | CE2 | TRP | A | 585 | −43.960 | −61.047 | 110.892 | 1.00 | 198.35 | C |
| ATOM | 4054 | CE3 | TRP | A | 585 | −43.094 | −63.236 | 110.331 | 1.00 | 198.31 | C |
| ATOM | 4055 | CZ2 | TRP | A | 585 | −45.293 | −61.482 | 110.776 | 1.00 | 198.40 | C |
| ATOM | 4056 | CZ3 | TRP | A | 585 | −44.429 | −63.676 | 110.220 | 1.00 | 198.48 | C |
| ATOM | 4057 | CH2 | TRP | A | 585 | −45.506 | −62.796 | 110.444 | 1.00 | 198.43 | C |
| ATOM | 4058 | N | TYR | A | 586 | −37.171 | −60.563 | 112.554 | 1.00 | 197.90 | N |
| ATOM | 4059 | CA | TYR | A | 586 | −36.164 | −60.892 | 113.557 | 1.00 | 197.86 | C |
| ATOM | 4060 | C | TYR | A | 586 | −35.523 | −59.575 | 113.974 | 1.00 | 197.91 | C |
| ATOM | 4061 | O | TYR | A | 586 | −34.330 | −59.511 | 114.274 | 1.00 | 197.78 | O |
| ATOM | 4062 | CB | TYR | A | 586 | −35.115 | −61.835 | 112.954 | 1.00 | 197.84 | C |
| ATOM | 4063 | CG | TYR | A | 586 | −35.657 | −63.205 | 112.598 | 1.00 | 197.90 | C |
| ATOM | 4064 | CD1 | TYR | A | 586 | −36.775 | −63.342 | 111.771 | 1.00 | 198.11 | C |
| ATOM | 4065 | CD2 | TYR | A | 586 | −35.052 | −64.366 | 113.074 | 1.00 | 197.88 | C |
| ATOM | 4066 | CE1 | TYR | A | 586 | −37.297 | −64.584 | 111.446 | 1.00 | 197.83 | C |
| ATOM | 4067 | CE2 | TYR | A | 586 | −35.565 | −65.631 | 112.741 | 1.00 | 197.95 | C |
| ATOM | 4068 | CZ | TYR | A | 586 | −36.691 | −65.724 | 111.923 | 1.00 | 197.78 | C |
| ATOM | 4069 | OH | TYR | A | 586 | −37.223 | −66.943 | 111.580 | 1.00 | 197.65 | O |
| ATOM | 4070 | N | LEU | A | 587 | −36.353 | −58.533 | 113.993 | 1.00 | 198.17 | N |
| ATOM | 4071 | CA | LEU | A | 587 | −35.932 | −57.133 | 114.124 | 1.00 | 198.48 | C |
| ATOM | 4072 | C | LEU | A | 587 | −34.967 | −56.893 | 115.272 | 1.00 | 198.71 | C |
| ATOM | 4073 | O | LEU | A | 587 | −33.751 | −56.862 | 115.070 | 1.00 | 198.68 | O |
| ATOM | 4074 | CB | LEU | A | 587 | −37.164 | −56.225 | 114.292 | 1.00 | 198.49 | C |
| ATOM | 4075 | CG | LEU | A | 587 | −37.055 | −54.722 | 114.016 | 1.00 | 198.11 | C |
| ATOM | 4076 | CD1 | LEU | A | 587 | −37.313 | −54.470 | 112.552 | 1.00 | 197.83 | C |
| ATOM | 4077 | CD2 | LEU | A | 587 | −38.054 | −53.946 | 114.857 | 1.00 | 197.56 | C |
| ATOM | 4078 | N | THR | A | 588 | −35.533 | −56.722 | 116.468 | 1.00 | 199.01 | N |
| ATOM | 4079 | CA | THR | A | 588 | −34.781 | −56.419 | 117.694 | 1.00 | 199.25 | C |
| ATOM | 4080 | C | THR | A | 588 | −34.368 | −57.695 | 118.419 | 1.00 | 199.02 | C |
| ATOM | 4081 | O | THR | A | 588 | −33.627 | −57.660 | 119.410 | 1.00 | 198.83 | O |
| ATOM | 4082 | CB | THR | A | 588 | −35.573 | −55.485 | 118.647 | 1.00 | 199.43 | C |
| ATOM | 4083 | OG1 | THR | A | 588 | −36.979 | −55.739 | 118.508 | 1.00 | 200.11 | O |
| ATOM | 4084 | CG2 | THR | A | 588 | −35.288 | −54.005 | 118.333 | 1.00 | 199.55 | C |
| ATOM | 4085 | N | GLU | A | 589 | −34.867 | −58.818 | 117.915 | 1.00 | 198.89 | N |
| ATOM | 4086 | CA | GLU | A | 589 | −34.262 | −60.096 | 118.196 | 1.00 | 198.95 | C |
| ATOM | 4087 | C | GLU | A | 589 | −32.758 | −59.931 | 117.971 | 1.00 | 198.82 | C |
| ATOM | 4088 | O | GLU | A | 589 | −31.970 | −60.115 | 118.895 | 1.00 | 198.83 | O |
| ATOM | 4089 | CB | GLU | A | 589 | −34.861 | −61.170 | 117.281 | 1.00 | 199.06 | C |
| ATOM | 4090 | CG | GLU | A | 589 | −33.994 | −62.422 | 117.061 | 1.00 | 199.84 | C |
| ATOM | 4091 | CD | GLU | A | 589 | −33.880 | −63.335 | 118.284 | 1.00 | 200.62 | C |
| ATOM | 4092 | OE1 | GLU | A | 589 | −33.252 | −64.414 | 118.163 | 1.00 | 200.71 | O |
| ATOM | 4093 | OE2 | GLU | A | 589 | −34.411 | −62.985 | 119.361 | 1.00 | 201.26 | O |
| ATOM | 4094 | N | ASN | A | 590 | −32.385 | −59.517 | 116.759 | 1.00 | 198.70 | N |
| ATOM | 4095 | CA | ASN | A | 590 | −30.988 | −59.353 | 116.358 | 1.00 | 198.55 | C |
| ATOM | 4096 | C | ASN | A | 590 | −30.159 | −58.323 | 117.130 | 1.00 | 198.67 | C |
| ATOM | 4097 | O | ASN | A | 590 | −28.933 | −58.342 | 117.017 | 1.00 | 198.57 | O |
| ATOM | 4098 | CB | ASN | A | 590 | −30.904 | −59.045 | 114.864 | 1.00 | 198.44 | C |
| ATOM | 4099 | CG | ASN | A | 590 | −31.011 | −60.282 | 114.005 | 1.00 | 198.24 | C |
| ATOM | 4100 | OD1 | ASN | A | 590 | −30.078 | −60.626 | 113.290 | 1.00 | 197.90 | O |
| ATOM | 4101 | ND2 | ASN | A | 590 | −32.149 | −60.954 | 114.065 | 1.00 | 198.25 | N |
| ATOM | 4102 | N | ILE | A | 591 | −30.810 | −57.433 | 117.893 | 1.00 | 198.94 | N |
| ATOM | 4103 | CA | ILE | A | 591 | −30.092 | −56.386 | 118.665 | 1.00 | 199.21 | C |
| ATOM | 4104 | C | ILE | A | 591 | −29.644 | −56.824 | 120.063 | 1.00 | 199.41 | C |
| ATOM | 4105 | O | ILE | A | 591 | −28.639 | −56.325 | 120.582 | 1.00 | 199.42 | O |
| ATOM | 4106 | CB | ILE | A | 591 | −30.843 | −54.998 | 118.756 | 1.00 | 199.16 | C |
| ATOM | 4107 | CG1 | ILE | A | 591 | −31.941 | −55.004 | 119.832 | 1.00 | 199.20 | C |
| ATOM | 4108 | CG2 | ILE | A | 591 | −31.359 | −54.550 | 117.388 | 1.00 | 199.07 | C |
| ATOM | 4109 | CD1 | ILE | A | 591 | −32.197 | −53.641 | 120.480 | 1.00 | 199.21 | C |
| ATOM | 4110 | N | GLN | A | 592 | −30.395 | −57.741 | 120.672 | 1.00 | 199.64 | N |
| ATOM | 4111 | CA | GLN | A | 592 | −29.947 | −58.399 | 121.895 | 1.00 | 199.91 | C |
| ATOM | 4112 | C | GLN | A | 592 | −28.808 | −59.354 | 121.558 | 1.00 | 200.02 | C |
| ATOM | 4113 | O | GLN | A | 592 | −27.939 | −59.622 | 122.384 | 1.00 | 200.11 | O |
| ATOM | 4114 | CB | GLN | A | 592 | −31.095 | −59.147 | 122.581 | 1.00 | 199.91 | C |
| ATOM | 4115 | CG | GLN | A | 592 | −31.810 | −58.344 | 123.667 | 1.00 | 200.05 | C |
| ATOM | 4116 | CD | GLN | A | 592 | −32.693 | −57.245 | 123.104 | 1.00 | 200.20 | C |
| ATOM | 4117 | OE1 | GLN | A | 592 | −33.643 | −57.514 | 122.370 | 1.00 | 200.11 | O |
| ATOM | 4118 | NE2 | GLN | A | 592 | −32.387 | −55.998 | 123.454 | 1.00 | 200.26 | N |
| ATOM | 4119 | N | ARG | A | 593 | −28.820 | −59.842 | 120.324 | 1.00 | 200.21 | N |
| ATOM | 4120 | CA | ARG | A | 593 | −27.816 | −60.766 | 119.822 | 1.00 | 200.47 | C |
| ATOM | 4121 | C | ARG | A | 593 | −26.473 | −60.066 | 119.601 | 1.00 | 200.29 | C |
| ATOM | 4122 | O | ARG | A | 593 | −25.755 | −59.756 | 120.555 | 1.00 | 200.17 | O |
| ATOM | 4123 | CB | ARG | A | 593 | −28.299 | −61.399 | 118.503 | 1.00 | 200.80 | C |
| ATOM | 4124 | CG | ARG | A | 593 | −29.672 | −62.083 | 118.566 | 1.00 | 201.85 | C |
| ATOM | 4125 | CD | ARG | A | 593 | −29.616 | −63.616 | 118.563 | 1.00 | 203.46 | C |
| ATOM | 4126 | NE | ARG | A | 593 | −28.516 | −64.156 | 119.360 | 1.00 | 204.72 | N |
| ATOM | 4127 | CZ | ARG | A | 593 | −27.541 | −64.924 | 118.882 | 1.00 | 205.38 | C |
| ATOM | 4128 | NH1 | ARG | A | 593 | −27.521 | −65.279 | 117.596 | 1.00 | 205.27 | N |
| ATOM | 4129 | NH2 | ARG | A | 593 | −26.584 | −65.347 | 119.702 | 1.00 | 205.73 | N |
| ATOM | 4130 | N | PHE | A | 594 | −26.167 | −59.805 | 118.331 | 1.00 | 200.21 | N |
| ATOM | 4131 | CA | PHE | A | 594 | −24.843 | −59.373 | 117.882 | 1.00 | 200.10 | C |
| ATOM | 4132 | C | PHE | A | 594 | −24.415 | −58.027 | 118.496 | 1.00 | 199.44 | C |
| ATOM | 4133 | O | PHE | A | 594 | −23.268 | −57.877 | 118.931 | 1.00 | 199.33 | O |
| ATOM | 4134 | CB | PHE | A | 594 | −24.795 | −59.364 | 116.333 | 1.00 | 200.63 | C |
| ATOM | 4135 | CG | PHE | A | 594 | −23.443 | −59.757 | 115.743 | 1.00 | 201.91 | C |
| ATOM | 4136 | CD1 | PHE | A | 594 | −22.750 | −58.878 | 114.893 | 1.00 | 202.89 | C |
| ATOM | 4137 | CD2 | PHE | A | 594 | −22.865 | −61.003 | 116.029 | 1.00 | 202.76 | C |
| ATOM | 4138 | CE1 | PHE | A | 594 | −21.499 | −59.227 | 114.343 | 1.00 | 202.97 | C |
| ATOM | 4139 | CE2 | PHE | A | 594 | −21.614 | −61.362 | 115.486 | 1.00 | 203.05 | C |
| ATOM | 4140 | CZ | PHE | A | 594 | −20.932 | −60.468 | 114.639 | 1.00 | 202.63 | C |
| ATOM | 4141 | N | LEU | A | 595 | −25.340 | −57.066 | 118.542 | 1.00 | 198.68 | N |
| ATOM | 4142 | CA | LEU | A | 595 | −25.093 | −55.778 | 119.183 | 1.00 | 197.95 | C |
| ATOM | 4143 | C | LEU | A | 595 | −24.913 | −56.050 | 120.665 | 1.00 | 197.74 | C |
| ATOM | 4144 | O | LEU | A | 595 | −25.766 | −56.705 | 121.274 | 1.00 | 197.81 | O |
| ATOM | 4145 | CB | LEU | A | 595 | −26.263 | −54.814 | 118.955 | 1.00 | 197.72 | C |
| ATOM | 4146 | CG | LEU | A | 595 | −26.088 | −53.298 | 119.119 | 1.00 | 197.15 | C |
| ATOM | 4147 | CD1 | LEU | A | 595 | −26.026 | −52.853 | 120.583 | 1.00 | 196.96 | C |
| ATOM | 4148 | CD2 | LEU | A | 595 | −24.884 | −52.787 | 118.338 | 1.00 | 196.64 | C |
| ATOM | 4149 | N | PRO | A | 596 | −23.793 | −55.568 | 121.246 | 1.00 | 197.38 | N |
| ATOM | 4150 | CA | PRO | A | 596 | −23.433 | −55.848 | 122.632 | 1.00 | 197.00 | C |
| ATOM | 4151 | C | PRO | A | 596 | −24.508 | −55.415 | 123.620 | 1.00 | 196.63 | C |
| ATOM | 4152 | O | PRO | A | 596 | −25.346 | −56.229 | 124.018 | 1.00 | 196.55 | O |
| ATOM | 4153 | CB | PRO | A | 596 | −22.152 | −55.027 | 122.830 | 1.00 | 197.02 | C |
| ATOM | 4154 | CG | PRO | A | 596 | −21.585 | −54.904 | 121.475 | 1.00 | 197.11 | C |
| ATOM | 4155 | CD | PRO | A | 596 | −22.777 | −54.720 | 120.596 | 1.00 | 197.36 | C |
| ATOM | 4156 | N | ASN | A | 597 | −24.489 | −54.142 | 123.998 | 1.00 | 196.22 | N |
| ATOM | 4157 | CA | ASN | A | 597 | −25.405 | −53.646 | 125.015 | 1.00 | 195.81 | C |
| ATOM | 4158 | C | ASN | A | 597 | −26.563 | −52.811 | 124.473 | 1.00 | 195.44 | C |
| ATOM | 4159 | O | ASN | A | 597 | −26.348 | −51.801 | 123.796 | 1.00 | 195.42 | O |
| ATOM | 4160 | CB | ASN | A | 597 | −24.666 | −52.919 | 126.163 | 1.00 | 195.79 | C |
| ATOM | 4161 | CG | ASN | A | 597 | −23.230 | −52.532 | 125.809 | 1.00 | 195.66 | C |
| ATOM | 4162 | OD1 | ASN | A | 597 | −22.911 | −52.205 | 124.663 | 1.00 | 196.05 | O |
| ATOM | 4163 | ND2 | ASN | A | 597 | −22.359 | −52.562 | 126.807 | 1.00 | 195.13 | N |
| ATOM | 4164 | N | PRO | A | 598 | −27.800 | −53.270 | 124.732 | 1.00 | 195.06 | N |
| ATOM | 4165 | CA | PRO | A | 598 | −28.972 | −52.417 | 124.656 | 1.00 | 194.81 | C |
| ATOM | 4166 | C | PRO | A | 598 | −29.144 | −51.586 | 125.938 | 1.00 | 194.64 | C |
| ATOM | 4167 | O | PRO | A | 598 | −30.210 | −51.005 | 126.138 | 1.00 | 194.58 | O |
| ATOM | 4168 | CB | PRO | A | 598 | −30.129 | −53.420 | 124.492 | 1.00 | 194.74 | C |
| ATOM | 4169 | CG | PRO | A | 598 | −29.493 | −54.784 | 124.395 | 1.00 | 194.74 | C |
| ATOM | 4170 | CD | PRO | A | 598 | −28.172 | −54.657 | 125.051 | 1.00 | 194.98 | C |
| ATOM | 4171 | N | ALA | A | 599 | −28.103 | −51.535 | 126.786 | 1.00 | 194.50 | N |
| ATOM | 4172 | CA | ALA | A | 599 | −28.076 | −50.707 | 128.021 | 1.00 | 194.26 | C |
| ATOM | 4173 | C | ALA | A | 599 | −28.391 | −49.243 | 127.703 | 1.00 | 194.08 | C |
| ATOM | 4174 | O | ALA | A | 599 | −28.643 | −48.415 | 128.599 | 1.00 | 193.91 | O |
| ATOM | 4175 | CB | ALA | A | 599 | −26.729 | −50.831 | 128.743 | 1.00 | 194.22 | C |
| ATOM | 4176 | N | GLY | A | 600 | −28.320 | −48.960 | 126.402 | 1.00 | 193.92 | N |
| ATOM | 4177 | CA | GLY | A | 600 | −28.965 | −47.831 | 125.752 | 1.00 | 193.61 | C |
| ATOM | 4178 | C | GLY | A | 600 | −29.698 | −48.428 | 124.565 | 1.00 | 193.28 | C |
| ATOM | 4179 | O | GLY | A | 600 | −29.139 | −48.528 | 123.473 | 1.00 | 193.12 | O |
| ATOM | 4180 | N | VAL | A | 601 | −30.944 | −48.842 | 124.799 | 1.00 | 193.13 | N |
| ATOM | 4181 | CA | VAL | A | 601 | −31.756 | −49.595 | 123.827 | 1.00 | 193.09 | C |
| ATOM | 4182 | C | VAL | A | 601 | −32.204 | −48.836 | 122.557 | 1.00 | 193.27 | C |
| ATOM | 4183 | O | VAL | A | 601 | −33.217 | −49.206 | 121.948 | 1.00 | 193.32 | O |
| ATOM | 4184 | CB | VAL | A | 601 | −33.015 | −50.240 | 124.500 | 1.00 | 193.00 | C |
| ATOM | 4185 | CG1 | VAL | A | 601 | −32.959 | −51.757 | 124.429 | 1.00 | 192.76 | C |
| ATOM | 4186 | CG2 | VAL | A | 601 | −33.211 | −49.743 | 125.933 | 1.00 | 192.57 | C |
| ATOM | 4187 | N | GLN | A | 602 | −31.441 | −47.810 | 122.158 | 1.00 | 193.31 | N |
| ATOM | 4188 | CA | GLN | A | 602 | −31.754 | −46.920 | 121.008 | 1.00 | 193.25 | C |
| ATOM | 4189 | C | GLN | A | 602 | −32.307 | −47.625 | 119.758 | 1.00 | 193.25 | C |
| ATOM | 4190 | O | GLN | A | 602 | −31.870 | −48.724 | 119.436 | 1.00 | 193.26 | O |
| ATOM | 4191 | CB | GLN | A | 602 | −30.519 | −46.080 | 120.624 | 1.00 | 193.24 | C |
| ATOM | 4192 | CG | GLN | A | 602 | −29.186 | −46.850 | 120.659 | 1.00 | 193.06 | C |
| ATOM | 4193 | CD | GLN | A | 602 | −28.370 | −46.705 | 119.357 | 1.00 | 192.70 | C |
| ATOM | 4194 | OE1 | GLN | A | 602 | −28.947 | −46.531 | 118.166 | 1.00 | 192.32 | O |
| ATOM | 4195 | NE2 | GLN | A | 602 | −26.925 | −46.842 | 119.660 | 1.00 | 192.41 | N |
| ATOM | 4196 | N | LEU | A | 603 | −33.263 | −46.989 | 119.071 | 1.00 | 193.32 | N |
| ATOM | 4197 | CA | LEU | A | 603 | −33.847 | −47.513 | 117.809 | 1.00 | 193.48 | C |
| ATOM | 4198 | C | LEU | A | 603 | −33.992 | −46.407 | 116.741 | 1.00 | 193.51 | C |
| ATOM | 4199 | O | LEU | A | 603 | −33.229 | −45.442 | 116.775 | 1.00 | 193.48 | O |
| ATOM | 4200 | CB | LEU | A | 603 | −35.199 | −48.213 | 118.066 | 1.00 | 193.49 | C |
| ATOM | 4201 | CG | LEU | A | 603 | −35.364 | −49.747 | 118.155 | 1.00 | 193.45 | C |
| ATOM | 4202 | CD1 | LEU | A | 603 | −34.696 | −50.473 | 116.984 | 1.00 | 193.16 | C |
| ATOM | 4203 | CD2 | LEU | A | 603 | −34.909 | −50.343 | 119.497 | 1.00 | 193.42 | C |
| ATOM | 4204 | N | GLU | A | 604 | −34.929 | −46.577 | 115.792 | 1.00 | 193.54 | N |
| ATOM | 4205 | CA | GLU | A | 604 | −35.395 | −45.510 | 114.864 | 1.00 | 193.64 | C |
| ATOM | 4206 | C | GLU | A | 604 | −34.422 | −44.318 | 114.744 | 1.00 | 193.68 | C |
| ATOM | 4207 | O | GLU | A | 604 | −34.435 | −43.411 | 115.581 | 1.00 | 193.79 | O |
| ATOM | 4208 | CB | GLU | A | 604 | −36.811 | −45.043 | 115.284 | 1.00 | 193.69 | C |
| ATOM | 4209 | CG | GLU | A | 604 | −37.286 | −43.659 | 114.762 | 1.00 | 193.82 | C |
| ATOM | 4210 | CD | GLU | A | 604 | −37.867 | −42.735 | 115.865 | 1.00 | 193.74 | C |
| ATOM | 4211 | OE1 | GLU | A | 604 | −38.443 | −43.235 | 116.859 | 1.00 | 193.68 | O |
| ATOM | 4212 | OE2 | GLU | A | 604 | −37.750 | −41.494 | 115.731 | 1.00 | 193.30 | O |
| ATOM | 4213 | N | ASP | A | 605 | −33.589 | −44.312 | 113.705 | 1.00 | 193.63 | N |
| ATOM | 4214 | CA | ASP | A | 605 | −32.442 | −43.395 | 113.663 | 1.00 | 193.60 | C |
| ATOM | 4215 | C | ASP | A | 605 | −32.045 | −43.002 | 112.234 | 1.00 | 193.75 | C |
| ATOM | 4216 | O | ASP | A | 605 | −32.327 | −43.741 | 111.300 | 1.00 | 193.70 | O |
| ATOM | 4217 | CB | ASP | A | 605 | −31.269 | −44.043 | 114.422 | 1.00 | 193.52 | C |
| ATOM | 4218 | CG | ASP | A | 605 | −29.976 | −43.267 | 114.304 | 1.00 | 193.35 | C |
| ATOM | 4219 | OD1 | ASP | A | 605 | −29.239 | −43.497 | 113.330 | 1.00 | 193.12 | O |
| ATOM | 4220 | OD2 | ASP | A | 605 | −29.683 | −42.441 | 115.192 | 1.00 | 193.62 | O |
| ATOM | 4221 | N | PRO | A | 606 | −31.433 | −41.811 | 112.052 | 1.00 | 194.02 | N |
| ATOM | 4222 | CA | PRO | A | 606 | −30.821 | −41.479 | 110.754 | 1.00 | 194.23 | C |
| ATOM | 4223 | C | PRO | A | 606 | −29.844 | −42.535 | 110.186 | 1.00 | 194.41 | C |
| ATOM | 4224 | O | PRO | A | 606 | −30.125 | −43.125 | 109.137 | 1.00 | 194.40 | O |
| ATOM | 4225 | CB | PRO | A | 606 | −30.099 | −40.151 | 111.036 | 1.00 | 194.22 | C |
| ATOM | 4226 | CG | PRO | A | 606 | −30.893 | −39.527 | 112.141 | 1.00 | 194.19 | C |
| ATOM | 4227 | CD | PRO | A | 606 | −31.351 | −40.677 | 113.001 | 1.00 | 194.10 | C |
| ATOM | 4228 | N | GLU | A | 607 | −28.727 | −42.770 | 110.877 | 1.00 | 194.65 | N |
| ATOM | 4229 | CA | GLU | A | 607 | −27.633 | −43.611 | 110.360 | 1.00 | 194.96 | C |
| ATOM | 4230 | C | GLU | A | 607 | −27.769 | −45.111 | 110.630 | 1.00 | 195.04 | C |
| ATOM | 4231 | O | GLU | A | 607 | −27.250 | −45.928 | 109.875 | 1.00 | 195.13 | O |
| ATOM | 4232 | CB | GLU | A | 607 | −26.267 | −43.114 | 110.862 | 1.00 | 195.01 | C |
| ATOM | 4233 | CG | GLU | A | 607 | −26.120 | −42.999 | 112.388 | 1.00 | 195.70 | C |
| ATOM | 4234 | CD | GLU | A | 607 | −26.501 | −41.622 | 112.945 | 1.00 | 196.55 | C |
| ATOM | 4235 | OE1 | GLU | A | 607 | −26.589 | −41.488 | 114.189 | 1.00 | 196.62 | O |
| ATOM | 4236 | OE2 | GLU | A | 607 | −26.708 | −40.675 | 112.152 | 1.00 | 196.86 | O |
| ATOM | 4237 | N | PHE | A | 608 | −28.453 | −45.461 | 111.712 | 1.00 | 195.24 | N |
| ATOM | 4238 | CA | PHE | A | 608 | −28.661 | −46.855 | 112.112 | 1.00 | 195.37 | C |
| ATOM | 4239 | C | PHE | A | 608 | −29.646 | −47.556 | 111.178 | 1.00 | 195.44 | C |
| ATOM | 4240 | O | PHE | A | 608 | −29.443 | −48.714 | 110.817 | 1.00 | 195.22 | O |
| ATOM | 4241 | CB | PHE | A | 608 | −29.111 | −46.891 | 113.583 | 1.00 | 195.46 | C |
| ATOM | 4242 | CG | PHE | A | 608 | −29.721 | −48.201 | 114.039 | 1.00 | 195.41 | C |
| ATOM | 4243 | CD1 | PHE | A | 608 | −28.933 | −49.337 | 114.222 | 1.00 | 195.19 | C |
| ATOM | 4244 | CD2 | PHE | A | 608 | −31.084 | −48.270 | 114.353 | 1.00 | 195.20 | C |
| ATOM | 4245 | CE1 | PHE | A | 608 | −29.502 | −50.533 | 114.672 | 1.00 | 194.96 | C |
| ATOM | 4246 | CE2 | PHE | A | 608 | −31.661 | −49.456 | 114.804 | 1.00 | 194.69 | C |
| ATOM | 4247 | CZ | PHE | A | 608 | −30.869 | −50.590 | 114.964 | 1.00 | 194.89 | C |
| ATOM | 4248 | N | GLN | A | 609 | −30.700 | −46.844 | 110.782 | 1.00 | 195.78 | N |
| ATOM | 4249 | CA | GLN | A | 609 | −31.635 | −47.347 | 109.783 | 1.00 | 196.24 | C |
| ATOM | 4250 | C | GLN | A | 609 | −30.871 | −47.608 | 108.489 | 1.00 | 196.75 | C |
| ATOM | 4251 | O | GLN | A | 609 | −31.037 | −48.658 | 107.872 | 1.00 | 196.96 | O |
| ATOM | 4252 | CB | GLN | A | 609 | −32.796 | −46.373 | 109.536 | 1.00 | 196.11 | C |
| ATOM | 4253 | CG | GLN | A | 609 | −33.709 | −46.107 | 110.739 | 1.00 | 195.84 | C |
| ATOM | 4254 | CD | GLN | A | 609 | −34.450 | −47.334 | 111.233 | 1.00 | 195.58 | C |
| ATOM | 4255 | OE1 | GLN | A | 609 | −34.917 | −48.155 | 110.444 | 1.00 | 195.51 | O |
| ATOM | 4256 | NE2 | GLN | A | 609 | −34.571 | −47.459 | 112.550 | 1.00 | 195.39 | N |
| ATOM | 4257 | N | ALA | A | 610 | −30.012 | −46.668 | 108.096 | 1.00 | 197.26 | N |
| ATOM | 4258 | CA | ALA | A | 610 | −29.140 | −46.856 | 106.929 | 1.00 | 197.71 | C |
| ATOM | 4259 | C | ALA | A | 610 | −28.074 | −47.957 | 107.147 | 1.00 | 197.97 | C |
| ATOM | 4260 | O | ALA | A | 610 | −27.492 | −48.481 | 106.189 | 1.00 | 197.87 | O |
| ATOM | 4261 | CB | ALA | A | 610 | −28.491 | −45.519 | 106.521 | 1.00 | 197.65 | C |
| ATOM | 4262 | N | SER | A | 611 | −27.854 | −48.318 | 108.409 | 1.00 | 198.32 | N |
| ATOM | 4263 | CA | SER | A | 611 | −26.786 | −49.233 | 108.787 | 1.00 | 198.75 | C |
| ATOM | 4264 | C | SER | A | 611 | −27.133 | −50.692 | 108.547 | 1.00 | 199.13 | C |
| ATOM | 4265 | O | SER | A | 611 | −26.300 | −51.576 | 108.747 | 1.00 | 198.96 | O |
| ATOM | 4266 | CB | SER | A | 611 | −26.432 | −49.024 | 110.256 | 1.00 | 198.72 | C |
| ATOM | 4267 | OG | SER | A | 611 | −25.463 | −49.956 | 110.682 | 1.00 | 199.01 | O |
| ATOM | 4268 | N | ASN | A | 612 | −28.364 | −50.945 | 108.120 | 1.00 | 199.92 | N |
| ATOM | 4269 | CA | ASN | A | 612 | −28.841 | −52.319 | 107.931 | 1.00 | 200.82 | C |
| ATOM | 4270 | C | ASN | A | 612 | −29.507 | −52.576 | 106.572 | 1.00 | 201.44 | C |
| ATOM | 4271 | O | ASN | A | 612 | −29.888 | −53.714 | 106.258 | 1.00 | 201.55 | O |
| ATOM | 4272 | CB | ASN | A | 612 | −29.780 | −52.719 | 109.072 | 1.00 | 200.73 | C |
| ATOM | 4273 | CG | ASN | A | 612 | −29.201 | −52.413 | 110.430 | 1.00 | 200.43 | C |
| ATOM | 4274 | OD1 | ASN | A | 612 | −28.275 | −53.081 | 110.890 | 1.00 | 200.14 | O |
| ATOM | 4275 | ND2 | ASN | A | 612 | −29.739 | −51.391 | 111.079 | 1.00 | 200.14 | N |
| ATOM | 4276 | N | ILE | A | 613 | −29.655 | −51.511 | 105.783 | 1.00 | 202.05 | N |
| ATOM | 4277 | CA | ILE | A | 613 | −30.092 | −51.625 | 104.399 | 1.00 | 202.64 | C |
| ATOM | 4278 | C | ILE | A | 613 | −28.965 | −52.345 | 103.657 | 1.00 | 202.86 | C |
| ATOM | 4279 | O | ILE | A | 613 | −27.962 | −51.736 | 103.279 | 1.00 | 202.95 | O |
| ATOM | 4280 | CB | ILE | A | 613 | −30.373 | −50.226 | 103.771 | 1.00 | 202.73 | C |
| ATOM | 4281 | CG1 | ILE | A | 613 | −31.292 | −49.386 | 104.678 | 1.00 | 202.88 | C |
| ATOM | 4282 | CG2 | ILE | A | 613 | −30.952 | −50.366 | 102.357 | 1.00 | 203.04 | C |
| ATOM | 4283 | CD1 | ILE | A | 613 | −31.464 | −47.907 | 104.254 | 1.00 | 202.85 | C |
| ATOM | 4284 | N | MET | A | 614 | −29.125 | −53.655 | 103.483 | 1.00 | 203.17 | N |
| ATOM | 4285 | CA | MET | A | 614 | −28.044 | −54.502 | 102.979 | 1.00 | 203.54 | C |
| ATOM | 4286 | C | MET | A | 614 | −28.121 | −54.798 | 101.479 | 1.00 | 203.72 | C |
| ATOM | 4287 | O | MET | A | 614 | −28.776 | −55.756 | 101.049 | 1.00 | 203.89 | O |
| ATOM | 4288 | CB | MET | A | 614 | −27.953 | −55.788 | 103.801 | 1.00 | 203.61 | C |
| ATOM | 4289 | CG | MET | A | 614 | −27.468 | −55.548 | 105.217 | 1.00 | 203.98 | C |
| ATOM | 4290 | SD | MET | A | 614 | −25.784 | −54.905 | 105.249 | 1.00 | 205.28 | S |
| ATOM | 4291 | CE | MET | A | 614 | −25.594 | −54.406 | 106.968 | 1.00 | 204.40 | C |
| ATOM | 4292 | N | HIS | A | 615 | −27.437 | −53.968 | 100.696 | 1.00 | 203.74 | N |
| ATOM | 4293 | CA | HIS | A | 615 | −27.405 | −54.105 | 99.253 | 1.00 | 203.80 | C |
| ATOM | 4294 | C | HIS | A | 615 | −26.437 | −55.229 | 98.907 | 1.00 | 203.90 | C |
| ATOM | 4295 | O | HIS | A | 615 | −25.229 | −55.065 | 99.074 | 1.00 | 203.90 | O |
| ATOM | 4296 | CB | HIS | A | 615 | −26.951 | −52.787 | 98.622 | 1.00 | 203.82 | C |
| ATOM | 4297 | CG | HIS | A | 615 | −27.641 | −51.571 | 99.174 | 1.00 | 204.36 | C |
| ATOM | 4298 | ND1 | HIS | A | 615 | −27.263 | −50.971 | 100.355 | 1.00 | 204.98 | N |
| ATOM | 4299 | CD2 | HIS | A | 615 | −28.666 | −50.827 | 98.691 | 1.00 | 204.89 | C |
| ATOM | 4300 | CE1 | HIS | A | 615 | −28.031 | −49.919 | 100.582 | 1.00 | 205.08 | C |
| ATOM | 4301 | NE2 | HIS | A | 615 | −28.895 | −49.812 | 99.589 | 1.00 | 204.89 | N |
| ATOM | 4302 | N | SER | A | 616 | −26.967 | −56.371 | 98.447 | 1.00 | 204.06 | N |
| ATOM | 4303 | CA | SER | A | 616 | −26.151 | −57.581 | 98.187 | 1.00 | 204.15 | C |
| ATOM | 4304 | C | SER | A | 616 | −26.632 | −58.450 | 97.026 | 1.00 | 204.32 | C |
| ATOM | 4305 | O | SER | A | 616 | −27.800 | −58.390 | 96.638 | 1.00 | 204.39 | O |
| ATOM | 4306 | CB | SER | A | 616 | −26.133 | −58.483 | 99.415 | 1.00 | 204.12 | C |
| ATOM | 4307 | OG | SER | A | 616 | −27.103 | −59.513 | 99.272 | 1.00 | 203.65 | O |
| ATOM | 4308 | N | ILE | A | 617 | −25.728 | −59.292 | 96.517 | 1.00 | 204.46 | N |
| ATOM | 4309 | CA | ILE | A | 617 | −26.072 | −60.335 | 95.534 | 1.00 | 204.57 | C |
| ATOM | 4310 | C | ILE | A | 617 | −25.939 | −61.743 | 96.108 | 1.00 | 204.52 | C |
| ATOM | 4311 | O | ILE | A | 617 | −24.832 | −62.272 | 96.242 | 1.00 | 204.46 | O |
| ATOM | 4312 | CB | ILE | A | 617 | −25.268 | −60.238 | 94.204 | 1.00 | 204.59 | C |
| ATOM | 4313 | CG1 | ILE | A | 617 | −23.797 | −59.839 | 94.436 | 1.00 | 204.72 | C |
| ATOM | 4314 | CG2 | ILE | A | 617 | −25.970 | −59.314 | 93.241 | 1.00 | 204.81 | C |
| ATOM | 4315 | CD1 | ILE | A | 617 | −23.473 | −58.368 | 94.205 | 1.00 | 204.99 | C |
| ATOM | 4316 | N | ASN | A | 618 | −27.085 | −62.339 | 96.426 | 1.00 | 204.58 | N |
| ATOM | 4317 | CA | ASN | A | 618 | −27.166 | −63.619 | 97.146 | 1.00 | 204.69 | C |
| ATOM | 4318 | C | ASN | A | 618 | −26.662 | −63.576 | 98.599 | 1.00 | 204.88 | C |
| ATOM | 4319 | O | ASN | A | 618 | −26.016 | −64.522 | 99.083 | 1.00 | 205.07 | O |
| ATOM | 4320 | CB | ASN | A | 618 | −26.485 | −64.743 | 96.362 | 1.00 | 204.51 | C |
| ATOM | 4321 | CG | ASN | A | 618 | −27.049 | −64.895 | 94.985 | 1.00 | 204.21 | C |
| ATOM | 4322 | OD1 | ASN | A | 618 | −27.732 | −63.997 | 94.484 | 1.00 | 203.79 | O |
| ATOM | 4323 | ND2 | ASN | A | 618 | −26.776 | −66.037 | 94.354 | 1.00 | 203.95 | N |
| ATOM | 4324 | N | GLY | A | 619 | −26.976 | −62.478 | 99.291 | 1.00 | 204.82 | N |
| ATOM | 4325 | CA | GLY | A | 619 | −26.570 | −62.300 | 100.681 | 1.00 | 204.60 | C |
| ATOM | 4326 | C | GLY | A | 619 | −25.115 | −61.899 | 100.792 | 1.00 | 204.42 | C |
| ATOM | 4327 | O | GLY | A | 619 | −24.734 | −61.178 | 101.704 | 1.00 | 204.51 | O |
| ATOM | 4328 | N | TYR | A | 620 | −24.294 | −62.374 | 99.863 | 1.00 | 204.20 | N |
| ATOM | 4329 | CA | TYR | A | 620 | −22.897 | −61.986 | 99.818 | 1.00 | 204.01 | C |
| ATOM | 4330 | C | TYR | A | 620 | −22.803 | −60.534 | 99.343 | 1.00 | 203.96 | C |
| ATOM | 4331 | O | TYR | A | 620 | −23.587 | −60.100 | 98.494 | 1.00 | 203.85 | O |
| ATOM | 4332 | CB | TYR | A | 620 | −22.093 | −62.948 | 98.934 | 1.00 | 203.96 | C |
| ATOM | 4333 | CG | TYR | A | 620 | −22.084 | −64.409 | 99.403 | 1.00 | 203.80 | C |
| ATOM | 4334 | CD1 | TYR | A | 620 | −22.314 | −64.754 | 100.739 | 1.00 | 203.44 | C |
| ATOM | 4335 | CD2 | TYR | A | 620 | −21.807 | −65.442 | 98.510 | 1.00 | 203.82 | C |
| ATOM | 4336 | CE1 | TYR | A | 620 | −22.286 | −66.086 | 101.157 | 1.00 | 203.17 | C |
| ATOM | 4337 | CE2 | TYR | A | 620 | −21.778 | −66.779 | 98.928 | 1.00 | 203.49 | C |
| ATOM | 4338 | CZ | TYR | A | 620 | −22.019 | −67.092 | 100.248 | 1.00 | 203.37 | C |
| ATOM | 4339 | OH | TYR | A | 620 | −21.982 | −68.411 | 100.653 | 1.00 | 203.50 | O |
| ATOM | 4340 | N | VAL | A | 621 | −21.866 | −59.786 | 99.923 | 1.00 | 203.94 | N |
| ATOM | 4341 | CA | VAL | A | 621 | −21.754 | −58.339 | 99.703 | 1.00 | 203.92 | C |
| ATOM | 4342 | C | VAL | A | 621 | −20.281 | −57.916 | 99.706 | 1.00 | 203.97 | C |
| ATOM | 4343 | O | VAL | A | 621 | −19.420 | −58.684 | 100.121 | 1.00 | 203.93 | O |
| ATOM | 4344 | CB | VAL | A | 621 | −22.570 | −57.538 | 100.777 | 1.00 | 203.88 | C |
| ATOM | 4345 | CG1 | VAL | A | 621 | −21.857 | −57.527 | 102.127 | 1.00 | 203.72 | C |
| ATOM | 4346 | CG2 | VAL | A | 621 | −22.847 | −56.120 | 100.316 | 1.00 | 203.64 | C |
| ATOM | 4347 | N | PHE | A | 622 | −20.001 | −56.700 | 99.237 | 1.00 | 204.01 | N |
| ATOM | 4348 | CA | PHE | A | 622 | −18.647 | −56.123 | 99.266 | 1.00 | 203.98 | C |
| ATOM | 4349 | C | PHE | A | 622 | −17.555 | −57.082 | 98.781 | 1.00 | 204.04 | C |
| ATOM | 4350 | O | PHE | A | 622 | −16.583 | −57.359 | 99.491 | 1.00 | 203.87 | O |
| ATOM | 4351 | CB | PHE | A | 622 | −18.314 | −55.576 | 100.663 | 1.00 | 203.85 | C |
| ATOM | 4352 | CG | PHE | A | 622 | −18.899 | −54.220 | 100.942 | 1.00 | 203.47 | C |
| ATOM | 4353 | CD1 | PHE | A | 622 | −19.947 | −54.075 | 101.838 | 1.00 | 202.91 | C |
| ATOM | 4354 | CD2 | PHE | A | 622 | −18.397 | −53.087 | 100.305 | 1.00 | 203.25 | C |
| ATOM | 4355 | CE1 | PHE | A | 622 | −20.485 | −52.826 | 102.099 | 1.00 | 202.91 | C |
| ATOM | 4356 | CE2 | PHE | A | 622 | −18.931 | −51.835 | 100.556 | 1.00 | 203.02 | C |
| ATOM | 4357 | CZ | PHE | A | 622 | −19.976 | −51.702 | 101.458 | 1.00 | 203.14 | C |
| ATOM | 4358 | N | ASP | A | 623 | −17.735 | −57.575 | 97.559 | 1.00 | 204.28 | N |
| ATOM | 4359 | CA | ASP | A | 623 | −16.838 | −58.554 | 96.958 | 1.00 | 204.59 | C |
| ATOM | 4360 | C | ASP | A | 623 | −16.712 | −59.788 | 97.842 | 1.00 | 204.62 | C |
| ATOM | 4361 | O | ASP | A | 623 | −15.665 | −60.440 | 97.866 | 1.00 | 204.70 | O |
| ATOM | 4362 | CB | ASP | A | 623 | −15.464 | −57.936 | 96.649 | 1.00 | 204.70 | C |
| ATOM | 4363 | CG | ASP | A | 623 | −15.541 | −56.822 | 95.606 | 1.00 | 205.43 | C |
| ATOM | 4364 | OD1 | ASP | A | 623 | −14.784 | −56.878 | 94.614 | 1.00 | 206.11 | O |
| ATOM | 4365 | OD2 | ASP | A | 623 | −16.360 | −55.888 | 95.768 | 1.00 | 206.16 | O |
| ATOM | 4366 | N | SER | A | 624 | −17.780 | −60.095 | 98.578 | 1.00 | 204.72 | N |
| ATOM | 4367 | CA | SER | A | 624 | −17.829 | −61.337 | 99.335 | 1.00 | 204.91 | C |
| ATOM | 4368 | C | SER | A | 624 | −18.267 | −62.432 | 98.413 | 1.00 | 204.99 | C |
| ATOM | 4369 | O | SER | A | 624 | −19.012 | −62.182 | 97.453 | 1.00 | 204.77 | O |
| ATOM | 4370 | CB | SER | A | 624 | −18.780 | −61.283 | 100.520 | 1.00 | 204.97 | C |
| ATOM | 4371 | OG | SER | A | 624 | −18.743 | −62.525 | 101.207 | 1.00 | 205.03 | O |
| ATOM | 4372 | N | LEU | A | 625 | −17.842 | −63.649 | 98.759 | 1.00 | 205.21 | N |
| ATOM | 4373 | CA | LEU | A | 625 | −17.757 | −64.757 | 97.804 | 1.00 | 205.31 | C |
| ATOM | 4374 | C | LEU | A | 625 | −18.343 | −64.394 | 96.403 | 1.00 | 205.53 | C |
| ATOM | 4375 | O | LEU | A | 625 | −19.544 | −64.093 | 96.208 | 1.00 | 205.18 | O |
| ATOM | 4376 | CB | LEU | A | 625 | −18.180 | −66.140 | 98.402 | 1.00 | 205.13 | C |
| ATOM | 4377 | CG | LEU | A | 625 | −17.309 | −66.923 | 99.431 | 1.00 | 204.01 | C |
| ATOM | 4378 | CD1 | LEU | A | 625 | −17.971 | −68.237 | 99.830 | 1.00 | 203.00 | C |
| ATOM | 4379 | CD2 | LEU | A | 625 | −15.888 | −67.219 | 98.972 | 1.00 | 202.64 | C |
| ATOM | 4380 | N | GLN | A | 626 | −17.400 | −64.369 | 95.465 | 1.00 | 205.83 | N |
| ATOM | 4381 | CA | GLN | A | 626 | −17.583 | −64.061 | 94.060 | 1.00 | 206.15 | C |
| ATOM | 4382 | C | GLN | A | 626 | −18.247 | −65.239 | 93.312 | 1.00 | 206.26 | C |
| ATOM | 4383 | O | GLN | A | 626 | −19.345 | −65.666 | 93.696 | 1.00 | 206.59 | O |
| ATOM | 4384 | CB | GLN | A | 626 | −16.205 | −63.694 | 93.477 | 1.00 | 206.20 | C |
| ATOM | 4385 | CG | GLN | A | 626 | −15.253 | −64.859 | 93.089 | 1.00 | 206.94 | C |
| ATOM | 4386 | CD | GLN | A | 626 | −15.113 | −65.957 | 94.148 | 1.00 | 207.83 | C |
| ATOM | 4387 | OE1 | GLN | A | 626 | −14.743 | −65.693 | 95.299 | 1.00 | 208.57 | O |
| ATOM | 4388 | NE2 | GLN | A | 626 | −15.399 | −67.200 | 93.751 | 1.00 | 207.68 | N |
| ATOM | 4389 | N | LEU | A | 627 | −17.601 | −65.741 | 92.251 | 1.00 | 206.07 | N |
| ATOM | 4390 | CA | LEU | A | 627 | −18.011 | −66.975 | 91.572 | 1.00 | 205.77 | C |
| ATOM | 4391 | C | LEU | A | 627 | −16.875 | −67.563 | 90.743 | 1.00 | 205.36 | C |
| ATOM | 4392 | O | LEU | A | 627 | −16.177 | −66.843 | 90.018 | 1.00 | 205.22 | O |
| ATOM | 4393 | CB | LEU | A | 627 | −19.228 | −66.735 | 90.671 | 1.00 | 205.96 | C |
| ATOM | 4394 | CG | LEU | A | 627 | −20.419 | −67.692 | 90.832 | 1.00 | 206.51 | C |
| ATOM | 4395 | CD1 | LEU | A | 627 | −21.637 | −67.128 | 90.086 | 1.00 | 206.56 | C |
| ATOM | 4396 | CD2 | LEU | A | 627 | −20.109 | −69.152 | 90.410 | 1.00 | 207.21 | C |
| ATOM | 4397 | N | SER | A | 628 | −16.711 | −68.879 | 90.852 | 1.00 | 204.90 | N |
| ATOM | 4398 | CA | SER | A | 628 | −15.754 | −69.608 | 90.031 | 1.00 | 204.40 | C |
| ATOM | 4399 | C | SER | A | 628 | −16.381 | −70.073 | 88.713 | 1.00 | 203.92 | C |
| ATOM | 4400 | O | SER | A | 628 | −17.436 | −70.726 | 88.705 | 1.00 | 203.90 | O |
| ATOM | 4401 | CB | SER | A | 628 | −15.181 | −70.802 | 90.799 | 1.00 | 204.49 | C |
| ATOM | 4402 | OG | SER | A | 628 | −13.989 | −71.271 | 90.183 | 1.00 | 204.82 | O |
| ATOM | 4403 | N | VAL | A | 629 | −15.724 | −69.696 | 87.614 | 1.00 | 203.20 | N |
| ATOM | 4404 | CA | VAL | A | 629 | −16.010 | −70.190 | 86.257 | 1.00 | 202.47 | C |
| ATOM | 4405 | C | VAL | A | 629 | −14.696 | −70.300 | 85.474 | 1.00 | 201.87 | C |
| ATOM | 4406 | O | VAL | A | 629 | −13.623 | −70.004 | 86.014 | 1.00 | 201.86 | O |
| ATOM | 4407 | CB | VAL | A | 629 | −17.025 | −69.295 | 85.480 | 1.00 | 202.49 | C |
| ATOM | 4408 | CG1 | VAL | A | 629 | −18.457 | −69.766 | 85.707 | 1.00 | 202.66 | C |
| ATOM | 4409 | CG2 | VAL | A | 629 | −16.869 | −67.825 | 85.849 | 1.00 | 202.52 | C |
| ATOM | 4410 | N | CYS | A | 630 | −14.774 | −70.743 | 84.217 | 1.00 | 201.07 | N |
| ATOM | 4411 | CA | CYS | A | 630 | −13.581 | −70.828 | 83.353 | 1.00 | 200.30 | C |
| ATOM | 4412 | C | CYS | A | 630 | −13.849 | −70.506 | 81.874 | 1.00 | 199.57 | C |
| ATOM | 4413 | O | CYS | A | 630 | −15.003 | −70.429 | 81.453 | 1.00 | 199.64 | O |
| ATOM | 4414 | CB | CYS | A | 630 | −12.831 | −72.160 | 83.538 | 1.00 | 200.40 | C |
| ATOM | 4415 | SG | CYS | A | 630 | −13.507 | −73.671 | 82.799 | 1.00 | 200.62 | S |
| ATOM | 4416 | N | LEU | A | 631 | −12.784 | −70.302 | 81.098 | 1.00 | 198.49 | N |
| ATOM | 4417 | CA | LEU | A | 631 | −12.931 | −69.764 | 79.745 | 1.00 | 197.39 | C |
| ATOM | 4418 | C | LEU | A | 631 | −13.227 | −70.856 | 78.731 | 1.00 | 196.91 | C |
| ATOM | 4419 | O | LEU | A | 631 | −12.826 | −71.996 | 78.917 | 1.00 | 196.75 | O |
| ATOM | 4420 | CB | LEU | A | 631 | −11.721 | −68.912 | 79.358 | 1.00 | 197.13 | C |
| ATOM | 4421 | CG | LEU | A | 631 | −10.662 | −69.434 | 78.412 | 1.00 | 196.21 | C |
| ATOM | 4422 | CD1 | LEU | A | 631 | −10.856 | −68.749 | 77.090 | 1.00 | 195.32 | C |
| ATOM | 4423 | CD2 | LEU | A | 631 | −9.321 | −69.092 | 78.991 | 1.00 | 195.99 | C |
| ATOM | 4424 | N | HIS | A | 632 | −13.936 | −70.481 | 77.667 | 1.00 | 196.45 | N |
| ATOM | 4425 | CA | HIS | A | 632 | −14.561 | −71.412 | 76.716 | 1.00 | 196.12 | C |
| ATOM | 4426 | C | HIS | A | 632 | −15.784 | −72.142 | 77.283 | 1.00 | 195.72 | C |
| ATOM | 4427 | O | HIS | A | 632 | −16.551 | −72.755 | 76.532 | 1.00 | 195.61 | O |
| ATOM | 4428 | CB | HIS | A | 632 | −13.555 | −72.404 | 76.124 | 1.00 | 196.35 | C |
| ATOM | 4429 | CG | HIS | A | 632 | −12.527 | −71.772 | 75.241 | 1.00 | 196.80 | C |
| ATOM | 4430 | ND1 | HIS | A | 632 | −12.634 | −70.478 | 74.777 | 1.00 | 197.40 | N |
| ATOM | 4431 | CD2 | HIS | A | 632 | −11.386 | −72.272 | 74.708 | 1.00 | 197.06 | C |
| ATOM | 4432 | CE1 | HIS | A | 632 | −11.593 | −70.201 | 74.012 | 1.00 | 197.81 | C |
| ATOM | 4433 | NE2 | HIS | A | 632 | −10.822 | −71.273 | 73.952 | 1.00 | 197.56 | N |
| ATOM | 4434 | N | GLU | A | 633 | −15.947 | −72.091 | 78.605 | 1.00 | 195.28 | N |
| ATOM | 4435 | CA | GLU | A | 633 | −17.187 | −72.511 | 79.261 | 1.00 | 194.99 | C |
| ATOM | 4436 | C | GLU | A | 633 | −18.329 | −71.641 | 78.763 | 1.00 | 194.45 | C |
| ATOM | 4437 | O | GLU | A | 633 | −18.149 | −70.446 | 78.520 | 1.00 | 194.46 | O |
| ATOM | 4438 | CB | GLU | A | 633 | −17.089 | −72.324 | 80.775 | 1.00 | 195.19 | C |
| ATOM | 4439 | CG | GLU | A | 633 | −17.025 | −73.587 | 81.631 | 1.00 | 195.40 | C |
| ATOM | 4440 | CD | GLU | A | 633 | −17.115 | −73.267 | 83.129 | 1.00 | 195.21 | C |
| ATOM | 4441 | OE1 | GLU | A | 633 | −17.573 | −72.154 | 83.492 | 1.00 | 194.95 | O |
| ATOM | 4442 | OE2 | GLU | A | 633 | −16.727 | −74.133 | 83.945 | 1.00 | 195.39 | O |
| ATOM | 4443 | N | VAL | A | 634 | −19.512 | −72.225 | 78.638 | 1.00 | 193.71 | N |
| ATOM | 4444 | CA | VAL | A | 634 | −20.633 | −71.481 | 78.095 | 1.00 | 192.98 | C |
| ATOM | 4445 | C | VAL | A | 634 | −21.954 | −71.992 | 78.677 | 1.00 | 192.57 | C |
| ATOM | 4446 | O | VAL | A | 634 | −22.257 | −73.186 | 78.572 | 1.00 | 192.75 | O |
| ATOM | 4447 | CB | VAL | A | 634 | −20.591 | −71.505 | 76.528 | 1.00 | 192.93 | C |
| ATOM | 4448 | CG1 | VAL | A | 634 | −20.719 | −72.931 | 75.972 | 1.00 | 192.91 | C |
| ATOM | 4449 | CG2 | VAL | A | 634 | −21.623 | −70.569 | 75.931 | 1.00 | 192.72 | C |
| ATOM | 4450 | N | ALA | A | 635 | −22.703 | −71.099 | 79.334 | 1.00 | 191.78 | N |
| ATOM | 4451 | CA | ALA | A | 635 | −24.072 | −71.394 | 79.804 | 1.00 | 191.01 | C |
| ATOM | 4452 | C | ALA | A | 635 | −24.862 | −70.139 | 80.152 | 1.00 | 190.53 | C |
| ATOM | 4453 | O | ALA | A | 635 | −24.316 | −69.034 | 80.183 | 1.00 | 190.26 | O |
| ATOM | 4454 | CB | ALA | A | 635 | −24.079 | −72.382 | 80.986 | 1.00 | 191.01 | C |
| ATOM | 4455 | N | TYR | A | 636 | −26.152 | −70.343 | 80.412 | 1.00 | 190.13 | N |
| ATOM | 4456 | CA | TYR | A | 636 | −27.113 | −69.285 | 80.699 | 1.00 | 189.95 | C |
| ATOM | 4457 | C | TYR | A | 636 | −26.933 | −68.725 | 82.095 | 1.00 | 190.12 | C |
| ATOM | 4458 | O | TYR | A | 636 | −26.682 | −69.494 | 83.026 | 1.00 | 190.47 | O |
| ATOM | 4459 | CB | TYR | A | 636 | −28.509 | −69.889 | 80.685 | 1.00 | 189.67 | C |
| ATOM | 4460 | CG | TYR | A | 636 | −29.165 | −70.080 | 79.346 | 1.00 | 189.07 | C |
| ATOM | 4461 | CD1 | TYR | A | 636 | −30.546 | −70.181 | 79.263 | 1.00 | 189.36 | C |
| ATOM | 4462 | CD2 | TYR | A | 636 | −28.432 | −70.164 | 78.173 | 1.00 | 188.22 | C |
| ATOM | 4463 | CE1 | TYR | A | 636 | −31.188 | −70.366 | 78.048 | 1.00 | 189.62 | C |
| ATOM | 4464 | CE2 | TYR | A | 636 | −29.063 | −70.346 | 76.943 | 1.00 | 188.59 | C |
| ATOM | 4465 | CZ | TYR | A | 636 | −30.447 | −70.446 | 76.888 | 1.00 | 189.18 | C |
| ATOM | 4466 | OH | TYR | A | 636 | −31.111 | −70.625 | 75.689 | 1.00 | 189.28 | O |
| ATOM | 4467 | N | TRP | A | 637 | −27.091 | −67.410 | 82.255 | 1.00 | 190.14 | N |
| ATOM | 4468 | CA | TRP | A | 637 | −27.340 | −66.835 | 83.584 | 1.00 | 190.39 | C |
| ATOM | 4469 | C | TRP | A | 637 | −28.822 | −66.484 | 83.758 | 1.00 | 189.97 | C |
| ATOM | 4470 | O | TRP | A | 637 | −29.455 | −65.988 | 82.828 | 1.00 | 190.22 | O |
| ATOM | 4471 | CB | TRP | A | 637 | −26.506 | −65.576 | 83.849 | 1.00 | 191.21 | C |
| ATOM | 4472 | CG | TRP | A | 637 | −25.018 | −65.762 | 84.074 | 1.00 | 192.38 | C |
| ATOM | 4473 | CD1 | TRP | A | 637 | −24.026 | −64.976 | 83.571 | 1.00 | 193.56 | C |
| ATOM | 4474 | CD2 | TRP | A | 637 | −24.359 | −66.767 | 84.871 | 1.00 | 193.47 | C |
| ATOM | 4475 | NE1 | TRP | A | 637 | −22.792 | −65.428 | 83.986 | 1.00 | 193.82 | N |
| ATOM | 4476 | CE2 | TRP | A | 637 | −22.965 | −66.524 | 84.783 | 1.00 | 193.49 | C |
| ATOM | 4477 | CE3 | TRP | A | 637 | −24.805 | −67.854 | 85.635 | 1.00 | 193.84 | C |
| ATOM | 4478 | CZ2 | TRP | A | 637 | −22.018 | −67.325 | 85.426 | 1.00 | 193.08 | C |
| ATOM | 4479 | CZ3 | TRP | A | 637 | −23.863 | −68.650 | 86.271 | 1.00 | 193.29 | C |
| ATOM | 4480 | CH2 | TRP | A | 637 | −22.486 | −68.381 | 86.166 | 1.00 | 193.30 | C |
| ATOM | 4481 | N | TYR | A | 638 | −29.375 | −66.739 | 84.942 | 1.00 | 189.20 | N |
| ATOM | 4482 | CA | TYR | A | 638 | −30.715 | −66.267 | 85.267 | 1.00 | 188.49 | C |
| ATOM | 4483 | C | TYR | A | 638 | −30.602 | −65.278 | 86.399 | 1.00 | 188.49 | C |
| ATOM | 4484 | O | TYR | A | 638 | −30.047 | −65.602 | 87.447 | 1.00 | 188.83 | O |
| ATOM | 4485 | CB | TYR | A | 638 | −31.623 | −67.416 | 85.673 | 1.00 | 188.01 | C |
| ATOM | 4486 | CG | TYR | A | 638 | −31.534 | −68.590 | 84.754 | 1.00 | 187.42 | C |
| ATOM | 4487 | CD1 | TYR | A | 638 | −32.020 | −68.524 | 83.460 | 1.00 | 187.25 | C |
| ATOM | 4488 | CD2 | TYR | A | 638 | −30.952 | −69.765 | 85.173 | 1.00 | 187.38 | C |
| ATOM | 4489 | CE1 | TYR | A | 638 | −31.927 | −69.609 | 82.605 | 1.00 | 187.11 | C |
| ATOM | 4490 | CE2 | TYR | A | 638 | −30.857 | −70.855 | 84.332 | 1.00 | 187.43 | C |
| ATOM | 4491 | CZ | TYR | A | 638 | −31.345 | −70.771 | 83.053 | 1.00 | 187.24 | C |
| ATOM | 4492 | OH | TYR | A | 638 | −31.250 | −71.864 | 82.234 | 1.00 | 187.35 | O |
| ATOM | 4493 | N | ILE | A | 639 | −31.124 | −64.073 | 86.184 | 1.00 | 188.32 | N |
| ATOM | 4494 | CA | ILE | A | 639 | −30.985 | −62.976 | 87.149 | 1.00 | 188.04 | C |
| ATOM | 4495 | C | ILE | A | 639 | −32.284 | −62.205 | 87.408 | 1.00 | 188.02 | C |
| ATOM | 4496 | O | ILE | A | 639 | −33.175 | −62.148 | 86.555 | 1.00 | 187.97 | O |
| ATOM | 4497 | CB | ILE | A | 639 | −29.825 | −62.021 | 86.766 | 1.00 | 187.88 | C |
| ATOM | 4498 | CG1 | ILE | A | 639 | −29.825 | −61.724 | 85.270 | 1.00 | 187.58 | C |
| ATOM | 4499 | CG2 | ILE | A | 639 | −28.505 | −62.652 | 87.103 | 1.00 | 187.94 | C |
| ATOM | 4500 | CD1 | ILE | A | 639 | −28.443 | −61.460 | 84.705 | 1.00 | 186.94 | C |
| ATOM | 4501 | N | LEU | A | 640 | −32.381 | −61.627 | 88.602 | 1.00 | 188.04 | N |
| ATOM | 4502 | CA | LEU | A | 640 | −33.598 | −60.938 | 89.047 | 1.00 | 188.19 | C |
| ATOM | 4503 | C | LEU | A | 640 | −33.340 | −59.828 | 90.061 | 1.00 | 188.18 | C |
| ATOM | 4504 | O | LEU | A | 640 | −32.323 | −59.838 | 90.757 | 1.00 | 188.32 | O |
| ATOM | 4505 | CB | LEU | A | 640 | −34.637 | −61.927 | 89.615 | 1.00 | 188.24 | C |
| ATOM | 4506 | CG | LEU | A | 640 | −34.412 | −63.419 | 89.942 | 1.00 | 188.62 | C |
| ATOM | 4507 | CD1 | LEU | A | 640 | −33.074 | −63.742 | 90.612 | 1.00 | 189.40 | C |
| ATOM | 4508 | CD2 | LEU | A | 640 | −35.559 | −63.940 | 90.802 | 1.00 | 188.22 | C |
| ATOM | 4509 | N | SER | A | 641 | −34.251 | −58.864 | 90.136 | 1.00 | 188.10 | N |
| ATOM | 4510 | CA | SER | A | 641 | −34.196 | −57.902 | 91.216 | 1.00 | 188.34 | C |
| ATOM | 4511 | C | SER | A | 641 | −35.349 | −58.144 | 92.142 | 1.00 | 188.81 | C |
| ATOM | 4512 | O | SER | A | 641 | −36.498 | −58.232 | 91.709 | 1.00 | 188.73 | O |
| ATOM | 4513 | CB | SER | A | 641 | −34.236 | −56.467 | 90.725 | 1.00 | 188.23 | C |
| ATOM | 4514 | OG | SER | A | 641 | −34.448 | −55.595 | 91.824 | 1.00 | 187.83 | O |
| ATOM | 4515 | N | ILE | A | 642 | −35.015 | −58.246 | 93.424 | 1.00 | 189.56 | N |
| ATOM | 4516 | CA | ILE | A | 642 | −35.970 | −58.549 | 94.481 | 1.00 | 190.23 | C |
| ATOM | 4517 | C | ILE | A | 642 | −35.574 | −57.761 | 95.740 | 1.00 | 191.15 | C |
| ATOM | 4518 | O | ILE | A | 642 | −34.377 | −57.599 | 96.014 | 1.00 | 190.97 | O |
| ATOM | 4519 | CB | ILE | A | 642 | −36.014 | −60.078 | 94.764 | 1.00 | 189.90 | C |
| ATOM | 4520 | CG1 | ILE | A | 642 | −37.448 | −60.605 | 94.810 | 1.00 | 188.82 | C |
| ATOM | 4521 | CG2 | ILE | A | 642 | −35.256 | −60.436 | 96.022 | 1.00 | 190.10 | C |
| ATOM | 4522 | CD1 | ILE | A | 642 | −37.830 | −61.365 | 93.586 | 1.00 | 187.00 | C |
| ATOM | 4523 | N | GLY | A | 643 | −36.578 | −57.252 | 96.469 | 1.00 | 192.29 | N |
| ATOM | 4524 | CA | GLY | A | 643 | −36.393 | −56.522 | 97.751 | 1.00 | 193.60 | C |
| ATOM | 4525 | C | GLY | A | 643 | −35.791 | −55.121 | 97.694 | 1.00 | 194.39 | C |
| ATOM | 4526 | O | GLY | A | 643 | −36.213 | −54.226 | 98.420 | 1.00 | 194.02 | O |
| ATOM | 4527 | N | ALA | A | 644 | −34.763 | −54.970 | 96.859 | 1.00 | 195.45 | N |
| ATOM | 4528 | CA | ALA | A | 644 | −34.190 | −53.686 | 96.470 | 1.00 | 196.31 | C |
| ATOM | 4529 | C | ALA | A | 644 | −35.163 | −53.011 | 95.544 | 1.00 | 196.93 | C |
| ATOM | 4530 | O | ALA | A | 644 | −35.089 | −53.182 | 94.327 | 1.00 | 196.94 | O |
| ATOM | 4531 | CB | ALA | A | 644 | −32.893 | −53.921 | 95.731 | 1.00 | 196.29 | C |
| ATOM | 4532 | N | GLN | A | 645 | −36.091 | −52.261 | 96.105 | 1.00 | 197.85 | N |
| ATOM | 4533 | CA | GLN | A | 645 | −37.130 | −51.670 | 95.276 | 1.00 | 199.11 | C |
| ATOM | 4534 | C | GLN | A | 645 | −37.065 | −50.150 | 95.294 | 1.00 | 199.13 | C |
| ATOM | 4535 | O | GLN | A | 645 | −35.993 | −49.588 | 95.532 | 1.00 | 199.07 | O |
| ATOM | 4536 | CB | GLN | A | 645 | −38.524 | −52.219 | 95.635 | 1.00 | 199.61 | C |
| ATOM | 4537 | CG | GLN | A | 645 | −38.623 | −52.925 | 96.994 | 1.00 | 202.09 | C |
| ATOM | 4538 | CD | GLN | A | 645 | −38.194 | −52.036 | 98.162 | 1.00 | 205.38 | C |
| ATOM | 4539 | OE1 | GLN | A | 645 | −38.622 | −50.876 | 98.280 | 1.00 | 207.23 | O |
| ATOM | 4540 | NE2 | GLN | A | 645 | −37.341 | −52.579 | 99.029 | 1.00 | 205.78 | N |
| ATOM | 4541 | N | THR | A | 646 | −38.199 | −49.498 | 95.023 | 1.00 | 199.43 | N |
| ATOM | 4542 | CA | THR | A | 646 | −38.264 | −48.043 | 94.856 | 1.00 | 199.76 | C |
| ATOM | 4543 | C | THR | A | 646 | −37.508 | −47.595 | 93.584 | 1.00 | 199.95 | C |
| ATOM | 4544 | O | THR | A | 646 | −37.806 | −46.522 | 93.024 | 1.00 | 200.29 | O |
| ATOM | 4545 | CB | THR | A | 646 | −37.773 | −47.274 | 96.143 | 1.00 | 199.87 | C |
| ATOM | 4546 | OG1 | THR | A | 646 | −38.514 | −47.722 | 97.289 | 1.00 | 199.89 | O |
| ATOM | 4547 | CG2 | THR | A | 646 | −37.910 | −45.734 | 96.002 | 1.00 | 199.71 | C |
| ATOM | 4548 | N | ASP | A | 647 | −36.563 | −48.431 | 93.125 | 1.00 | 199.81 | N |
| ATOM | 4549 | CA | ASP | A | 647 | −35.671 | −48.109 | 91.991 | 1.00 | 199.67 | C |
| ATOM | 4550 | C | ASP | A | 647 | −35.446 | −49.282 | 91.032 | 1.00 | 198.91 | C |
| ATOM | 4551 | O | ASP | A | 647 | −35.929 | −50.395 | 91.267 | 1.00 | 199.02 | O |
| ATOM | 4552 | CB | ASP | A | 647 | −34.309 | −47.616 | 92.510 | 1.00 | 200.20 | C |
| ATOM | 4553 | CG | ASP | A | 647 | −33.655 | −46.564 | 91.591 | 1.00 | 201.85 | C |
| ATOM | 4554 | OD1 | ASP | A | 647 | −34.355 | −45.980 | 90.717 | 1.00 | 203.83 | O |
| ATOM | 4555 | OD2 | ASP | A | 647 | −32.434 | −46.312 | 91.764 | 1.00 | 203.19 | O |
| ATOM | 4556 | N | PHE | A | 648 | −34.713 | −49.021 | 89.949 | 1.00 | 197.97 | N |
| ATOM | 4557 | CA | PHE | A | 648 | −34.303 | −50.076 | 89.028 | 1.00 | 197.00 | C |
| ATOM | 4558 | C | PHE | A | 648 | −32.794 | −50.322 | 89.035 | 1.00 | 196.49 | C |
| ATOM | 4559 | O | PHE | A | 648 | −32.024 | −49.577 | 89.643 | 1.00 | 196.35 | O |
| ATOM | 4560 | CB | PHE | A | 648 | −34.825 | −49.818 | 87.605 | 1.00 | 196.83 | C |
| ATOM | 4561 | CG | PHE | A | 648 | −34.222 | −48.619 | 86.925 | 1.00 | 196.21 | C |
| ATOM | 4562 | CD1 | PHE | A | 648 | −32.896 | −48.625 | 86.504 | 1.00 | 195.73 | C |
| ATOM | 4563 | CD2 | PHE | A | 648 | −34.997 | −47.502 | 86.664 | 1.00 | 195.81 | C |
| ATOM | 4564 | CE1 | PHE | A | 648 | −32.351 | −47.531 | 85.864 | 1.00 | 195.66 | C |
| ATOM | 4565 | CE2 | PHE | A | 648 | −34.458 | −46.402 | 86.025 | 1.00 | 195.79 | C |
| ATOM | 4566 | CZ | PHE | A | 648 | −33.134 | −46.415 | 85.625 | 1.00 | 195.92 | C |
| ATOM | 4567 | N | LEU | A | 649 | −32.385 | −51.373 | 88.340 | 1.00 | 195.85 | N |
| ATOM | 4568 | CA | LEU | A | 649 | −30.994 | −51.750 | 88.288 | 1.00 | 195.40 | C |
| ATOM | 4569 | C | LEU | A | 649 | −30.493 | −51.999 | 86.875 | 1.00 | 194.99 | C |
| ATOM | 4570 | O | LEU | A | 649 | −30.840 | −53.012 | 86.265 | 1.00 | 195.19 | O |
| ATOM | 4571 | CB | LEU | A | 649 | −30.799 | −53.021 | 89.094 | 1.00 | 195.47 | C |
| ATOM | 4572 | CG | LEU | A | 649 | −30.726 | −52.860 | 90.598 | 1.00 | 195.92 | C |
| ATOM | 4573 | CD1 | LEU | A | 649 | −30.885 | −54.228 | 91.242 | 1.00 | 196.75 | C |
| ATOM | 4574 | CD2 | LEU | A | 649 | −29.407 | −52.189 | 90.996 | 1.00 | 196.27 | C |
| ATOM | 4575 | N | SER | A | 650 | −29.680 | −51.090 | 86.347 | 1.00 | 194.21 | N |
| ATOM | 4576 | CA | SER | A | 650 | −28.873 | −51.434 | 85.183 | 1.00 | 193.46 | C |
| ATOM | 4577 | C | SER | A | 650 | −27.721 | −52.270 | 85.732 | 1.00 | 193.04 | C |
| ATOM | 4578 | O | SER | A | 650 | −27.109 | −51.873 | 86.720 | 1.00 | 192.87 | O |
| ATOM | 4579 | CB | SER | A | 650 | −28.407 | −50.187 | 84.420 | 1.00 | 193.41 | C |
| ATOM | 4580 | OG | SER | A | 650 | −28.420 | −49.028 | 85.232 | 1.00 | 193.09 | O |
| ATOM | 4581 | N | VAL | A | 651 | −27.468 | −53.443 | 85.138 | 1.00 | 192.61 | N |
| ATOM | 4582 | CA | VAL | A | 651 | −26.526 | −54.426 | 85.723 | 1.00 | 192.28 | C |
| ATOM | 4583 | C | VAL | A | 651 | −25.274 | −54.726 | 84.891 | 1.00 | 192.25 | C |
| ATOM | 4584 | O | VAL | A | 651 | −25.328 | −54.768 | 83.661 | 1.00 | 192.25 | O |
| ATOM | 4585 | CB | VAL | A | 651 | −27.208 | −55.745 | 86.135 | 1.00 | 192.12 | C |
| ATOM | 4586 | CG1 | VAL | A | 651 | −28.344 | −55.476 | 87.104 | 1.00 | 191.95 | C |
| ATOM | 4587 | CG2 | VAL | A | 651 | −27.699 | −56.495 | 84.928 | 1.00 | 192.09 | C |
| ATOM | 4588 | N | PHE | A | 652 | −24.168 | −54.974 | 85.592 | 1.00 | 192.20 | N |
| ATOM | 4589 | CA | PHE | A | 652 | −22.829 | −54.768 | 85.046 | 1.00 | 192.25 | C |
| ATOM | 4590 | C | PHE | A | 652 | −22.253 | −55.970 | 84.286 | 1.00 | 192.08 | C |
| ATOM | 4591 | O | PHE | A | 652 | −22.765 | −56.311 | 83.227 | 1.00 | 191.99 | O |
| ATOM | 4592 | CB | PHE | A | 652 | −21.888 | −54.239 | 86.148 | 1.00 | 192.49 | C |
| ATOM | 4593 | CG | PHE | A | 652 | −20.821 | −53.285 | 85.655 | 1.00 | 193.15 | C |
| ATOM | 4594 | CD1 | PHE | A | 652 | −19.641 | −53.108 | 86.387 | 1.00 | 193.69 | C |
| ATOM | 4595 | CD2 | PHE | A | 652 | −20.982 | −52.566 | 84.463 | 1.00 | 194.09 | C |
| ATOM | 4596 | CE1 | PHE | A | 652 | −18.627 | −52.224 | 85.942 | 1.00 | 194.27 | C |
| ATOM | 4597 | CE2 | PHE | A | 652 | −19.974 | −51.679 | 83.999 | 1.00 | 194.65 | C |
| ATOM | 4598 | CZ | PHE | A | 652 | −18.794 | −51.508 | 84.744 | 1.00 | 194.18 | C |
| ATOM | 4599 | N | PHE | A | 653 | −21.216 | −56.604 | 84.841 | 1.00 | 192.04 | N |
| ATOM | 4600 | CA | PHE | A | 653 | −20.343 | −57.589 | 84.159 | 1.00 | 191.98 | C |
| ATOM | 4601 | C | PHE | A | 653 | −19.226 | −56.877 | 83.417 | 1.00 | 191.72 | C |
| ATOM | 4602 | O | PHE | A | 653 | −19.441 | −56.317 | 82.346 | 1.00 | 191.55 | O |
| ATOM | 4603 | CB | PHE | A | 653 | −21.082 | −58.517 | 83.176 | 1.00 | 192.25 | C |
| ATOM | 4604 | CG | PHE | A | 653 | −21.986 | −59.548 | 83.828 | 1.00 | 192.87 | C |
| ATOM | 4605 | CD1 | PHE | A | 653 | −21.631 | −60.182 | 85.016 | 1.00 | 193.32 | C |
| ATOM | 4606 | CD2 | PHE | A | 653 | −23.187 | −59.920 | 83.209 | 1.00 | 193.24 | C |
| ATOM | 4607 | CE1 | PHE | A | 653 | −22.481 | −61.144 | 85.596 | 1.00 | 193.57 | C |
| ATOM | 4608 | CE2 | PHE | A | 653 | −24.039 | −60.884 | 83.777 | 1.00 | 193.01 | C |
| ATOM | 4609 | CZ | PHE | A | 653 | −23.687 | −61.494 | 84.970 | 1.00 | 193.06 | C |
| ATOM | 4610 | N | SER | A | 654 | −18.030 | −56.910 | 83.989 | 1.00 | 191.71 | N |
| ATOM | 4611 | CA | SER | A | 654 | −16.883 | −56.225 | 83.404 | 1.00 | 191.86 | C |
| ATOM | 4612 | C | SER | A | 654 | −16.386 | −56.965 | 82.166 | 1.00 | 191.85 | C |
| ATOM | 4613 | O | SER | A | 654 | −16.168 | −58.178 | 82.216 | 1.00 | 191.88 | O |
| ATOM | 4614 | CB | SER | A | 654 | −15.761 | −56.084 | 84.440 | 1.00 | 191.94 | C |
| ATOM | 4615 | OG | SER | A | 654 | −14.761 | −55.163 | 84.025 | 1.00 | 191.97 | O |
| ATOM | 4616 | N | GLY | A | 655 | −16.238 | −56.230 | 81.059 | 1.00 | 191.80 | N |
| ATOM | 4617 | CA | GLY | A | 655 | −15.716 | −56.763 | 79.785 | 1.00 | 191.66 | C |
| ATOM | 4618 | C | GLY | A | 655 | −16.548 | −57.840 | 79.088 | 1.00 | 191.46 | C |
| ATOM | 4619 | O | GLY | A | 655 | −16.276 | −58.232 | 77.937 | 1.00 | 191.45 | O |
| ATOM | 4620 | N | TYR | A | 656 | −17.564 | −58.327 | 79.787 | 1.00 | 191.05 | N |
| ATOM | 4621 | CA | TYR | A | 656 | −18.386 | −59.370 | 79.250 | 1.00 | 190.60 | C |
| ATOM | 4622 | C | TYR | A | 656 | −19.658 | −58.841 | 78.650 | 1.00 | 190.32 | C |
| ATOM | 4623 | O | TYR | A | 656 | −20.142 | −57.766 | 79.006 | 1.00 | 190.16 | O |
| ATOM | 4624 | CB | TYR | A | 656 | −18.681 | −60.400 | 80.312 | 1.00 | 190.78 | C |
| ATOM | 4625 | CG | TYR | A | 656 | −17.713 | −61.537 | 80.261 | 1.00 | 191.12 | C |
| ATOM | 4626 | CD1 | TYR | A | 656 | −17.531 | −62.265 | 79.088 | 1.00 | 191.52 | C |
| ATOM | 4627 | CD2 | TYR | A | 656 | −16.975 | −61.894 | 81.384 | 1.00 | 191.71 | C |
| ATOM | 4628 | CE1 | TYR | A | 656 | −16.637 | −63.330 | 79.036 | 1.00 | 192.25 | C |
| ATOM | 4629 | CE2 | TYR | A | 656 | −16.074 | −62.958 | 81.349 | 1.00 | 192.09 | C |
| ATOM | 4630 | CZ | TYR | A | 656 | −15.910 | −63.675 | 80.172 | 1.00 | 192.09 | C |
| ATOM | 4631 | OH | TYR | A | 656 | −15.020 | −64.731 | 80.133 | 1.00 | 191.81 | O |
| ATOM | 4632 | N | THR | A | 657 | −20.189 | −59.621 | 77.723 | 1.00 | 190.03 | N |
| ATOM | 4633 | CA | THR | A | 657 | −21.333 | −59.219 | 76.944 | 1.00 | 189.76 | C |
| ATOM | 4634 | C | THR | A | 657 | −22.261 | −60.407 | 76.807 | 1.00 | 189.53 | C |
| ATOM | 4635 | O | THR | A | 657 | −21.804 | −61.549 | 76.698 | 1.00 | 189.24 | O |
| ATOM | 4636 | CB | THR | A | 657 | −20.887 | −58.747 | 75.551 | 1.00 | 189.87 | C |
| ATOM | 4637 | OG1 | THR | A | 657 | −19.782 | −57.842 | 75.682 | 1.00 | 189.74 | O |
| ATOM | 4638 | CG2 | THR | A | 657 | −22.023 | −58.056 | 74.829 | 1.00 | 189.94 | C |
| ATOM | 4639 | N | PHE | A | 658 | −23.561 | −60.131 | 76.801 | 1.00 | 189.49 | N |
| ATOM | 4640 | CA | PHE | A | 658 | −24.553 | −61.191 | 76.721 | 1.00 | 189.69 | C |
| ATOM | 4641 | C | PHE | A | 658 | −25.594 | −61.062 | 75.611 | 1.00 | 189.78 | C |
| ATOM | 4642 | O | PHE | A | 658 | −25.778 | −59.988 | 75.025 | 1.00 | 189.71 | O |
| ATOM | 4643 | CB | PHE | A | 658 | −25.251 | −61.364 | 78.060 | 1.00 | 189.78 | C |
| ATOM | 4644 | CG | PHE | A | 658 | −25.712 | −60.091 | 78.663 | 1.00 | 189.88 | C |
| ATOM | 4645 | CD1 | PHE | A | 658 | −24.971 | −59.482 | 79.662 | 1.00 | 190.05 | C |
| ATOM | 4646 | CD2 | PHE | A | 658 | −26.896 | −59.500 | 78.245 | 1.00 | 190.56 | C |
| ATOM | 4647 | CE1 | PHE | A | 658 | −25.400 | −58.294 | 80.242 | 1.00 | 190.66 | C |
| ATOM | 4648 | CE2 | PHE | A | 658 | −27.339 | −58.308 | 78.815 | 1.00 | 191.17 | C |
| ATOM | 4649 | CZ | PHE | A | 658 | −26.589 | −57.703 | 79.818 | 1.00 | 190.84 | C |
| ATOM | 4650 | N | LYS | A | 659 | −26.255 | −62.195 | 75.345 | 1.00 | 189.92 | N |
| ATOM | 4651 | CA | LYS | A | 659 | −27.300 | −62.335 | 74.329 | 1.00 | 190.12 | C |
| ATOM | 4652 | C | LYS | A | 659 | −28.646 | −62.541 | 75.035 | 1.00 | 190.47 | C |
| ATOM | 4653 | O | LYS | A | 659 | −29.150 | −63.659 | 75.184 | 1.00 | 190.60 | O |
| ATOM | 4654 | CB | LYS | A | 659 | −26.975 | −63.481 | 73.337 | 1.00 | 190.11 | C |
| ATOM | 4655 | CG | LYS | A | 659 | −27.968 | −63.704 | 72.161 | 1.00 | 189.80 | C |
| ATOM | 4656 | CD | LYS | A | 659 | −27.645 | −64.991 | 71.386 | 1.00 | 189.62 | C |
| ATOM | 4657 | CE | LYS | A | 659 | −28.834 | −65.515 | 70.592 | 1.00 | 188.89 | C |
| ATOM | 4658 | NZ | LYS | A | 659 | −29.004 | −64.776 | 69.318 | 1.00 | 188.81 | N |
| ATOM | 4659 | N | HIS | A | 660 | −29.203 | −61.430 | 75.489 | 1.00 | 190.71 | N |
| ATOM | 4660 | CA | HIS | A | 660 | −30.514 | −61.402 | 76.087 | 1.00 | 190.97 | C |
| ATOM | 4661 | C | HIS | A | 660 | −31.464 | −60.910 | 75.013 | 1.00 | 191.27 | C |
| ATOM | 4662 | O | HIS | A | 660 | −31.150 | −59.936 | 74.329 | 1.00 | 191.16 | O |
| ATOM | 4663 | CB | HIS | A | 660 | −30.457 | −60.475 | 77.297 | 1.00 | 190.93 | C |
| ATOM | 4664 | CG | HIS | A | 660 | −31.661 | −59.614 | 77.473 | 1.00 | 191.09 | C |
| ATOM | 4665 | ND1 | HIS | A | 660 | −32.776 | −60.030 | 78.166 | 1.00 | 191.14 | N |
| ATOM | 4666 | CD2 | HIS | A | 660 | −31.914 | −58.347 | 77.071 | 1.00 | 191.63 | C |
| ATOM | 4667 | CE1 | HIS | A | 660 | −33.674 | −59.062 | 78.169 | 1.00 | 191.76 | C |
| ATOM | 4668 | NE2 | HIS | A | 660 | −33.176 | −58.028 | 77.513 | 1.00 | 192.23 | N |
| ATOM | 4669 | N | LYS | A | 661 | −32.597 | −61.599 | 74.850 | 1.00 | 191.85 | N |
| ATOM | 4670 | CA | LYS | A | 661 | −33.634 | −61.255 | 73.853 | 1.00 | 192.68 | C |
| ATOM | 4671 | C | LYS | A | 661 | −33.157 | −61.462 | 72.401 | 1.00 | 192.88 | C |
| ATOM | 4672 | O | LYS | A | 661 | −33.620 | −60.790 | 71.467 | 1.00 | 192.83 | O |
| ATOM | 4673 | CB | LYS | A | 661 | −34.239 | −59.836 | 74.097 | 1.00 | 192.90 | C |
| ATOM | 4674 | CG | LYS | A | 661 | −33.368 | −58.584 | 73.696 | 1.00 | 193.40 | C |
| ATOM | 4675 | CD | LYS | A | 661 | −33.955 | −57.227 | 74.192 | 1.00 | 193.26 | C |
| ATOM | 4676 | CE | LYS | A | 661 | −32.953 | −56.048 | 74.072 | 1.00 | 193.54 | C |
| ATOM | 4677 | NZ | LYS | A | 661 | −33.485 | −54.740 | 74.607 | 1.00 | 193.49 | N |
| ATOM | 4678 | N | MET | A | 662 | −32.249 | −62.422 | 72.230 | 1.00 | 193.23 | N |
| ATOM | 4679 | CA | MET | A | 662 | −31.486 | −62.639 | 70.973 | 1.00 | 193.57 | C |
| ATOM | 4680 | C | MET | A | 662 | −31.007 | −61.371 | 70.213 | 1.00 | 193.10 | C |
| ATOM | 4681 | O | MET | A | 662 | −31.068 | −61.284 | 68.989 | 1.00 | 192.96 | O |
| ATOM | 4682 | CB | MET | A | 662 | −32.130 | −63.713 | 70.059 | 1.00 | 193.98 | C |
| ATOM | 4683 | CG | MET | A | 662 | −33.623 | −63.560 | 69.769 | 1.00 | 195.82 | C |
| ATOM | 4684 | SD | MET | A | 662 | −33.994 | −62.549 | 68.309 | 1.00 | 199.65 | S |
| ATOM | 4685 | CE | MET | A | 662 | −34.108 | −63.787 | 66.999 | 1.00 | 199.17 | C |
| ATOM | 4686 | N | VAL | A | 663 | −30.533 | −60.398 | 70.980 | 1.00 | 192.79 | N |
| ATOM | 4687 | CA | VAL | A | 663 | −29.714 | −59.302 | 70.489 | 1.00 | 192.63 | C |
| ATOM | 4688 | C | VAL | A | 663 | −28.437 | −59.453 | 71.313 | 1.00 | 192.54 | C |
| ATOM | 4689 | O | VAL | A | 663 | −28.344 | −60.383 | 72.099 | 1.00 | 192.48 | O |
| ATOM | 4690 | CB | VAL | A | 663 | −30.422 | −57.945 | 70.733 | 1.00 | 192.66 | C |
| ATOM | 4691 | CG1 | VAL | A | 663 | −29.496 | −56.755 | 70.493 | 1.00 | 192.73 | C |
| ATOM | 4692 | CG2 | VAL | A | 663 | −31.638 | −57.831 | 69.842 | 1.00 | 192.86 | C |
| ATOM | 4693 | N | TYR | A | 664 | −27.437 | −58.598 | 71.128 | 1.00 | 192.56 | N |
| ATOM | 4694 | CA | TYR | A | 664 | −26.309 | −58.583 | 72.060 | 1.00 | 192.61 | C |
| ATOM | 4695 | C | TYR | A | 664 | −26.197 | −57.192 | 72.689 | 1.00 | 192.13 | C |
| ATOM | 4696 | O | TYR | A | 664 | −25.977 | −56.206 | 71.974 | 1.00 | 192.27 | O |
| ATOM | 4697 | CB | TYR | A | 664 | −25.004 | −58.997 | 71.368 | 1.00 | 193.31 | C |
| ATOM | 4698 | CG | TYR | A | 664 | −24.844 | −60.493 | 71.095 | 1.00 | 194.69 | C |
| ATOM | 4699 | CD1 | TYR | A | 664 | −25.759 | −61.185 | 70.288 | 1.00 | 196.55 | C |
| ATOM | 4700 | CD2 | TYR | A | 664 | −23.753 | −61.215 | 71.607 | 1.00 | 195.83 | C |
| ATOM | 4701 | CE1 | TYR | A | 664 | −25.613 | −62.573 | 70.008 | 1.00 | 196.80 | C |
| ATOM | 4702 | CE2 | TYR | A | 664 | −23.597 | −62.610 | 71.335 | 1.00 | 196.33 | C |
| ATOM | 4703 | CZ | TYR | A | 664 | −24.539 | −63.279 | 70.534 | 1.00 | 195.83 | C |
| ATOM | 4704 | OH | TYR | A | 664 | −24.424 | −64.631 | 70.248 | 1.00 | 194.57 | O |
| ATOM | 4705 | N | GLU | A | 665 | −26.402 | −57.118 | 74.009 | 1.00 | 191.29 | N |
| ATOM | 4706 | CA | GLU | A | 665 | −26.278 | −55.880 | 74.782 | 1.00 | 190.51 | C |
| ATOM | 4707 | C | GLU | A | 665 | −25.225 | −56.141 | 75.819 | 1.00 | 190.15 | C |
| ATOM | 4708 | O | GLU | A | 665 | −24.879 | −57.294 | 76.050 | 1.00 | 189.91 | O |
| ATOM | 4709 | CB | GLU | A | 665 | −27.588 | −55.515 | 75.490 | 1.00 | 190.39 | C |
| ATOM | 4710 | CG | GLU | A | 665 | −28.746 | −55.073 | 74.581 | 1.00 | 190.66 | C |
| ATOM | 4711 | CD | GLU | A | 665 | −28.486 | −53.771 | 73.804 | 1.00 | 191.12 | C |
| ATOM | 4712 | OE1 | GLU | A | 665 | −29.275 | −53.474 | 72.876 | 1.00 | 191.16 | O |
| ATOM | 4713 | OE2 | GLU | A | 665 | −27.508 | −53.046 | 74.108 | 1.00 | 191.01 | O |
| ATOM | 4714 | N | ASP | A | 666 | −24.714 | −55.081 | 76.442 | 1.00 | 190.11 | N |
| ATOM | 4715 | CA | ASP | A | 666 | −23.717 | −55.225 | 77.520 | 1.00 | 190.19 | C |
| ATOM | 4716 | C | ASP | A | 666 | −24.242 | −54.869 | 78.920 | 1.00 | 190.05 | C |
| ATOM | 4717 | O | ASP | A | 666 | −23.531 | −55.049 | 79.916 | 1.00 | 190.00 | O |
| ATOM | 4718 | CB | ASP | A | 666 | −22.387 | −54.493 | 77.197 | 1.00 | 190.23 | C |
| ATOM | 4719 | CG | ASP | A | 666 | −22.312 | −53.054 | 77.754 | 1.00 | 190.73 | C |
| ATOM | 4720 | OD1 | ASP | A | 666 | −21.331 | −52.340 | 77.440 | 1.00 | 190.85 | O |
| ATOM | 4721 | OD2 | ASP | A | 666 | −23.210 | −52.618 | 78.504 | 1.00 | 191.71 | O |
| ATOM | 4722 | N | THR | A | 667 | −25.472 | −54.359 | 78.991 | 1.00 | 189.84 | N |
| ATOM | 4723 | CA | THR | A | 667 | −26.077 | −54.013 | 80.269 | 1.00 | 189.69 | C |
| ATOM | 4724 | C | THR | A | 667 | −27.545 | −54.303 | 80.291 | 1.00 | 189.84 | C |
| ATOM | 4725 | O | THR | A | 667 | −28.280 | −53.964 | 79.370 | 1.00 | 189.75 | O |
| ATOM | 4726 | CB | THR | A | 667 | −25.878 | −52.550 | 80.620 | 1.00 | 189.63 | C |
| ATOM | 4727 | OG1 | THR | A | 667 | −24.485 | −52.315 | 80.833 | 1.00 | 189.89 | O |
| ATOM | 4728 | CG2 | THR | A | 667 | −26.637 | −52.202 | 81.891 | 1.00 | 189.46 | C |
| ATOM | 4729 | N | LEU | A | 668 | −27.958 | −54.921 | 81.383 | 1.00 | 190.30 | N |
| ATOM | 4730 | CA | LEU | A | 668 | −29.320 | −55.358 | 81.551 | 1.00 | 190.97 | C |
| ATOM | 4731 | C | LEU | A | 668 | −30.034 | −54.400 | 82.489 | 1.00 | 191.51 | C |
| ATOM | 4732 | O | LEU | A | 668 | −29.608 | −54.214 | 83.626 | 1.00 | 191.64 | O |
| ATOM | 4733 | CB | LEU | A | 668 | −29.325 | −56.771 | 82.138 | 1.00 | 190.75 | C |
| ATOM | 4734 | CG | LEU | A | 668 | −30.441 | −57.769 | 81.843 | 1.00 | 190.49 | C |
| ATOM | 4735 | CD1 | LEU | A | 668 | −30.291 | −58.937 | 82.783 | 1.00 | 189.90 | C |
| ATOM | 4736 | CD2 | LEU | A | 668 | −31.834 | −57.156 | 81.970 | 1.00 | 190.61 | C |
| ATOM | 4737 | N | THR | A | 669 | −31.110 | −53.786 | 82.007 | 1.00 | 192.17 | N |
| ATOM | 4738 | CA | THR | A | 669 | −31.936 | −52.932 | 82.855 | 1.00 | 192.84 | C |
| ATOM | 4739 | C | THR | A | 669 | −33.007 | −53.772 | 83.543 | 1.00 | 193.24 | C |
| ATOM | 4740 | O | THR | A | 669 | −33.744 | −54.509 | 82.891 | 1.00 | 193.25 | O |
| ATOM | 4741 | CB | THR | A | 669 | −32.543 | −51.741 | 82.078 | 1.00 | 192.85 | C |
| ATOM | 4742 | OG1 | THR | A | 669 | −32.949 | −52.169 | 80.770 | 1.00 | 193.47 | O |
| ATOM | 4743 | CG2 | THR | A | 669 | −31.513 | −50.621 | 81.935 | 1.00 | 192.93 | C |
| ATOM | 4744 | N | LEU | A | 670 | −33.064 | −53.665 | 84.868 | 1.00 | 193.97 | N |
| ATOM | 4745 | CA | LEU | A | 670 | −33.914 | −54.523 | 85.704 | 1.00 | 194.67 | C |
| ATOM | 4746 | C | LEU | A | 670 | −34.730 | −53.781 | 86.763 | 1.00 | 195.12 | C |
| ATOM | 4747 | O | LEU | A | 670 | −34.174 | −53.141 | 87.674 | 1.00 | 195.23 | O |
| ATOM | 4748 | CB | LEU | A | 670 | −33.077 | −55.595 | 86.411 | 1.00 | 194.63 | C |
| ATOM | 4749 | CG | LEU | A | 670 | −32.680 | −56.888 | 85.703 | 1.00 | 195.02 | C |
| ATOM | 4750 | CD1 | LEU | A | 670 | −32.126 | −57.856 | 86.726 | 1.00 | 195.61 | C |
| ATOM | 4751 | CD2 | LEU | A | 670 | −33.843 | −57.534 | 84.973 | 1.00 | 195.81 | C |
| ATOM | 4752 | N | PHE | A | 671 | −36.049 | −53.926 | 86.655 | 1.00 | 195.47 | N |
| ATOM | 4753 | CA | PHE | A | 671 | −37.026 | −53.335 | 87.578 | 1.00 | 195.92 | C |
| ATOM | 4754 | C | PHE | A | 671 | −37.031 | −53.885 | 89.061 | 1.00 | 196.28 | C |
| ATOM | 4755 | O | PHE | A | 671 | −35.962 | −53.981 | 89.674 | 1.00 | 196.41 | O |
| ATOM | 4756 | CB | PHE | A | 671 | −38.392 | −53.350 | 86.881 | 1.00 | 195.26 | C |
| ATOM | 4757 | CG | PHE | A | 671 | −38.645 | −52.136 | 86.014 | 1.00 | 194.25 | C |
| ATOM | 4758 | CD1 | PHE | A | 671 | −39.543 | −52.188 | 84.965 | 1.00 | 192.96 | C |
| ATOM | 4759 | CD2 | PHE | A | 671 | −37.997 | −50.932 | 86.265 | 1.00 | 193.15 | C |
| ATOM | 4760 | CE1 | PHE | A | 671 | −39.797 | −51.059 | 84.191 | 1.00 | 191.98 | C |
| ATOM | 4761 | CE2 | PHE | A | 671 | −38.244 | −49.810 | 85.490 | 1.00 | 192.46 | C |
| ATOM | 4762 | CZ | PHE | A | 671 | −39.146 | −49.877 | 84.454 | 1.00 | 192.65 | C |
| ATOM | 4763 | N | PRO | A | 672 | −38.209 | −54.112 | 89.690 | 1.00 | 196.71 | N |
| ATOM | 4764 | CA | PRO | A | 672 | −38.304 | −54.938 | 90.903 | 1.00 | 197.00 | C |
| ATOM | 4765 | C | PRO | A | 672 | −39.141 | −56.242 | 90.790 | 1.00 | 197.59 | C |
| ATOM | 4766 | O | PRO | A | 672 | −39.214 | −57.031 | 91.741 | 1.00 | 197.40 | O |
| ATOM | 4767 | CB | PRO | A | 672 | −38.987 | −53.975 | 91.873 | 1.00 | 197.23 | C |
| ATOM | 4768 | CG | PRO | A | 672 | −39.760 | −52.946 | 90.915 | 1.00 | 197.09 | C |
| ATOM | 4769 | CD | PRO | A | 672 | −39.474 | −53.394 | 89.500 | 1.00 | 196.71 | C |
| ATOM | 4770 | N | PHE | A | 673 | −39.791 | −56.437 | 89.650 | 1.00 | 198.26 | N |
| ATOM | 4771 | CA | PHE | A | 673 | −40.380 | −57.725 | 89.304 | 1.00 | 199.11 | C |
| ATOM | 4772 | C | PHE | A | 673 | −39.263 | −58.604 | 88.820 | 1.00 | 198.72 | C |
| ATOM | 4773 | O | PHE | A | 673 | −39.369 | −59.833 | 88.794 | 1.00 | 198.47 | O |
| ATOM | 4774 | CB | PHE | A | 673 | −41.433 | −57.561 | 88.165 | 1.00 | 200.31 | C |
| ATOM | 4775 | CG | PHE | A | 673 | −40.861 | −57.522 | 86.686 | 1.00 | 202.03 | C |
| ATOM | 4776 | CD1 | PHE | A | 673 | −40.533 | −58.720 | 85.979 | 1.00 | 203.13 | C |
| ATOM | 4777 | CD2 | PHE | A | 673 | −40.750 | −56.295 | 85.978 | 1.00 | 202.52 | C |
| ATOM | 4778 | CE1 | PHE | A | 673 | −40.044 | −58.684 | 84.621 | 1.00 | 201.99 | C |
| ATOM | 4779 | CE2 | PHE | A | 673 | −40.273 | −56.255 | 84.626 | 1.00 | 201.55 | C |
| ATOM | 4780 | CZ | PHE | A | 673 | −39.920 | −57.451 | 83.953 | 1.00 | 201.55 | C |
| ATOM | 4781 | N | SER | A | 674 | −38.175 | −57.920 | 88.484 | 1.00 | 198.43 | N |
| ATOM | 4782 | CA | SER | A | 674 | −37.411 | −58.193 | 87.271 | 1.00 | 198.17 | C |
| ATOM | 4783 | C | SER | A | 674 | −36.849 | −59.585 | 87.074 | 1.00 | 197.76 | C |
| ATOM | 4784 | O | SER | A | 674 | −35.793 | −59.929 | 87.602 | 1.00 | 197.86 | O |
| ATOM | 4785 | CB | SER | A | 674 | −36.355 | −57.107 | 87.023 | 1.00 | 198.33 | C |
| ATOM | 4786 | OG | SER | A | 674 | −36.815 | −56.149 | 86.070 | 1.00 | 198.32 | O |
| ATOM | 4787 | N | GLY | A | 675 | −37.574 | −60.369 | 86.287 | 1.00 | 197.11 | N |
| ATOM | 4788 | CA | GLY | A | 675 | −37.092 | −61.663 | 85.866 | 1.00 | 196.49 | C |
| ATOM | 4789 | C | GLY | A | 675 | −36.608 | −61.543 | 84.448 | 1.00 | 195.85 | C |
| ATOM | 4790 | O | GLY | A | 675 | −37.403 | −61.268 | 83.552 | 1.00 | 195.98 | O |
| ATOM | 4791 | N | GLU | A | 676 | −35.304 | −61.713 | 84.252 | 1.00 | 195.11 | N |
| ATOM | 4792 | CA | GLU | A | 676 | −34.746 | −61.782 | 82.912 | 1.00 | 194.69 | C |
| ATOM | 4793 | C | GLU | A | 676 | −33.620 | −62.797 | 82.793 | 1.00 | 193.77 | C |
| ATOM | 4794 | O | GLU | A | 676 | −32.924 | −63.063 | 83.766 | 1.00 | 193.82 | O |
| ATOM | 4795 | CB | GLU | A | 676 | −34.309 | −60.406 | 82.414 | 1.00 | 195.02 | C |
| ATOM | 4796 | CG | GLU | A | 676 | −35.467 | −59.521 | 81.903 | 1.00 | 197.27 | C |
| ATOM | 4797 | CD | GLU | A | 676 | −36.212 | −60.065 | 80.655 | 1.00 | 200.28 | C |
| ATOM | 4798 | OE1 | GLU | A | 676 | −36.198 | −61.299 | 80.375 | 1.00 | 200.93 | O |
| ATOM | 4799 | OE2 | GLU | A | 676 | −36.838 | −59.231 | 79.954 | 1.00 | 201.78 | O |
| ATOM | 4800 | N | THR | A | 677 | −33.440 | −63.321 | 81.579 | 1.00 | 192.71 | N |
| ATOM | 4801 | CA | THR | A | 677 | −32.642 | −64.524 | 81.304 | 1.00 | 191.40 | C |
| ATOM | 4802 | C | THR | A | 677 | −31.584 | −64.318 | 80.209 | 1.00 | 190.68 | C |
| ATOM | 4803 | O | THR | A | 677 | −31.915 | −64.315 | 79.028 | 1.00 | 190.57 | O |
| ATOM | 4804 | CB | THR | A | 677 | −33.584 | −65.658 | 80.838 | 1.00 | 191.36 | C |
| ATOM | 4805 | OG1 | THR | A | 677 | −34.681 | −65.786 | 81.756 | 1.00 | 191.17 | O |
| ATOM | 4806 | CG2 | THR | A | 677 | −32.838 | −66.974 | 80.699 | 1.00 | 190.70 | C |
| ATOM | 4807 | N | VAL | A | 678 | −30.317 | −64.171 | 80.584 | 1.00 | 189.80 | N |
| ATOM | 4808 | CA | VAL | A | 678 | −29.263 | −63.911 | 79.587 | 1.00 | 189.13 | C |
| ATOM | 4809 | C | VAL | A | 678 | −28.497 | −65.154 | 79.158 | 1.00 | 188.62 | C |
| ATOM | 4810 | O | VAL | A | 678 | −28.698 | −66.236 | 79.703 | 1.00 | 188.71 | O |
| ATOM | 4811 | CB | VAL | A | 678 | −28.242 | −62.848 | 80.055 | 1.00 | 189.13 | C |
| ATOM | 4812 | CG1 | VAL | A | 678 | −28.957 | −61.609 | 80.558 | 1.00 | 189.54 | C |
| ATOM | 4813 | CG2 | VAL | A | 678 | −27.308 | −63.409 | 81.120 | 1.00 | 189.06 | C |
| ATOM | 4814 | N | PHE | A | 679 | −27.614 | −64.983 | 78.178 | 1.00 | 187.94 | N |
| ATOM | 4815 | CA | PHE | A | 679 | −26.739 | −66.058 | 77.727 | 1.00 | 187.44 | C |
| ATOM | 4816 | C | PHE | A | 679 | −25.375 | −65.475 | 77.405 | 1.00 | 187.14 | C |
| ATOM | 4817 | O | PHE | A | 679 | −25.283 | −64.335 | 76.969 | 1.00 | 187.05 | O |
| ATOM | 4818 | CB | PHE | A | 679 | −27.356 | −66.781 | 76.526 | 1.00 | 187.41 | C |
| ATOM | 4819 | CG | PHE | A | 679 | −26.503 | −67.893 | 75.950 | 1.00 | 187.14 | C |
| ATOM | 4820 | CD1 | PHE | A | 679 | −26.590 | −68.207 | 74.597 | 1.00 | 187.06 | C |
| ATOM | 4821 | CD2 | PHE | A | 679 | −25.630 | −68.626 | 76.747 | 1.00 | 187.03 | C |
| ATOM | 4822 | CE1 | PHE | A | 679 | −25.823 | −69.223 | 74.048 | 1.00 | 187.12 | C |
| ATOM | 4823 | CE2 | PHE | A | 679 | −24.854 | −69.640 | 76.205 | 1.00 | 186.97 | C |
| ATOM | 4824 | CZ | PHE | A | 679 | −24.952 | −69.942 | 74.856 | 1.00 | 187.09 | C |
| ATOM | 4825 | N | MET | A | 680 | −24.326 | −66.270 | 77.616 | 1.00 | 186.90 | N |
| ATOM | 4826 | CA | MET | A | 680 | −22.970 | −65.754 | 77.685 | 1.00 | 186.63 | C |
| ATOM | 4827 | C | MET | A | 680 | −21.882 | −66.740 | 77.255 | 1.00 | 187.01 | C |
| ATOM | 4828 | O | MET | A | 680 | −21.796 | −67.863 | 77.766 | 1.00 | 187.03 | O |
| ATOM | 4829 | CB | MET | A | 680 | −22.703 | −65.321 | 79.115 | 1.00 | 186.52 | C |
| ATOM | 4830 | CG | MET | A | 680 | −21.644 | −64.284 | 79.254 | 1.00 | 186.07 | C |
| ATOM | 4831 | SD | MET | A | 680 | −21.693 | −63.661 | 80.925 | 1.00 | 185.74 | S |
| ATOM | 4832 | CE | MET | A | 680 | −23.291 | −62.855 | 80.946 | 1.00 | 185.24 | C |
| ATOM | 4833 | N | SER | A | 681 | −21.041 | −66.289 | 76.326 | 1.00 | 187.33 | N |
| ATOM | 4834 | CA | SER | A | 681 | −19.847 | −67.021 | 75.909 | 1.00 | 187.64 | C |
| ATOM | 4835 | C | SER | A | 681 | −18.668 | −66.590 | 76.795 | 1.00 | 187.91 | C |
| ATOM | 4836 | O | SER | A | 681 | −18.092 | −65.519 | 76.593 | 1.00 | 188.02 | O |
| ATOM | 4837 | CB | SER | A | 681 | −19.566 | −66.758 | 74.415 | 1.00 | 187.63 | C |
| ATOM | 4838 | OG | SER | A | 681 | −18.370 | −67.376 | 73.954 | 1.00 | 187.33 | O |
| ATOM | 4839 | N | MET | A | 682 | −18.330 | −67.412 | 77.788 | 1.00 | 188.14 | N |
| ATOM | 4840 | CA | MET | A | 682 | −17.248 | −67.096 | 78.723 | 1.00 | 188.54 | C |
| ATOM | 4841 | C | MET | A | 682 | −15.884 | −67.338 | 78.099 | 1.00 | 188.84 | C |
| ATOM | 4842 | O | MET | A | 682 | −15.412 | −68.471 | 78.076 | 1.00 | 188.94 | O |
| ATOM | 4843 | CB | MET | A | 682 | −17.365 | −67.946 | 79.985 | 1.00 | 188.51 | C |
| ATOM | 4844 | CG | MET | A | 682 | −18.703 | −67.866 | 80.686 | 1.00 | 189.17 | C |
| ATOM | 4845 | SD | MET | A | 682 | −19.010 | −66.221 | 81.342 | 1.00 | 189.81 | S |
| ATOM | 4846 | CE | MET | A | 682 | −17.595 | −66.025 | 82.419 | 1.00 | 190.14 | C |
| ATOM | 4847 | N | GLU | A | 683 | −15.254 | −66.282 | 77.589 | 1.00 | 189.31 | N |
| ATOM | 4848 | CA | GLU | A | 683 | −13.897 | −66.389 | 77.020 | 1.00 | 189.84 | C |
| ATOM | 4849 | C | GLU | A | 683 | −13.038 | −65.113 | 77.150 | 1.00 | 189.80 | C |
| ATOM | 4850 | O | GLU | A | 683 | −12.218 | −64.809 | 76.277 | 1.00 | 189.89 | O |
| ATOM | 4851 | CB | GLU | A | 683 | −13.908 | −66.934 | 75.566 | 1.00 | 190.04 | C |
| ATOM | 4852 | CG | GLU | A | 683 | −15.169 | −66.661 | 74.726 | 1.00 | 190.98 | C |
| ATOM | 4853 | CD | GLU | A | 683 | −15.400 | −65.182 | 74.435 | 1.00 | 192.40 | C |
| ATOM | 4854 | OE1 | GLU | A | 683 | −15.768 | −64.433 | 75.367 | 1.00 | 192.68 | O |
| ATOM | 4855 | OE2 | GLU | A | 683 | −15.231 | −64.768 | 73.265 | 1.00 | 193.41 | O |
| ATOM | 4856 | N | ASN | A | 684 | −13.221 | −64.388 | 78.252 | 1.00 | 189.75 | N |
| ATOM | 4857 | CA | ASN | A | 684 | −12.392 | −63.231 | 78.566 | 1.00 | 189.80 | C |
| ATOM | 4858 | C | ASN | A | 684 | −11.708 | −63.431 | 79.920 | 1.00 | 189.98 | C |
| ATOM | 4859 | O | ASN | A | 684 | −12.263 | −63.067 | 80.951 | 1.00 | 190.01 | O |
| ATOM | 4860 | CB | ASN | A | 684 | −13.226 | −61.944 | 78.549 | 1.00 | 189.71 | C |
| ATOM | 4861 | CG | ASN | A | 684 | −12.475 | −60.760 | 77.953 | 1.00 | 189.79 | C |
| ATOM | 4862 | OD1 | ASN | A | 684 | −11.348 | −60.893 | 77.474 | 1.00 | 190.04 | O |
| ATOM | 4863 | ND2 | ASN | A | 684 | −13.110 | −59.594 | 77.968 | 1.00 | 189.86 | N |
| ATOM | 4864 | N | PRO | A | 685 | −10.504 | −64.029 | 79.915 | 1.00 | 190.20 | N |
| ATOM | 4865 | CA | PRO | A | 685 | −9.731 | −64.402 | 81.101 | 1.00 | 190.48 | C |
| ATOM | 4866 | C | PRO | A | 685 | −9.300 | −63.253 | 82.007 | 1.00 | 190.89 | C |
| ATOM | 4867 | O | PRO | A | 685 | −8.572 | −62.357 | 81.573 | 1.00 | 190.71 | O |
| ATOM | 4868 | CB | PRO | A | 685 | −8.490 | −65.070 | 80.505 | 1.00 | 190.51 | C |
| ATOM | 4869 | CG | PRO | A | 685 | −8.388 | −64.521 | 79.131 | 1.00 | 190.44 | C |
| ATOM | 4870 | CD | PRO | A | 685 | −9.800 | −64.409 | 78.680 | 1.00 | 190.25 | C |
| ATOM | 4871 | N | GLY | A | 686 | −9.741 | −63.307 | 83.264 | 1.00 | 191.61 | N |
| ATOM | 4872 | CA | GLY | A | 686 | −9.332 | −62.346 | 84.298 | 1.00 | 192.51 | C |
| ATOM | 4873 | C | GLY | A | 686 | −10.345 | −62.027 | 85.394 | 1.00 | 193.03 | C |
| ATOM | 4874 | O | GLY | A | 686 | −11.459 | −62.570 | 85.416 | 1.00 | 193.00 | O |
| ATOM | 4875 | N | LEU | A | 687 | −9.941 | −61.154 | 86.320 | 1.00 | 193.54 | N |
| ATOM | 4876 | CA | LEU | A | 687 | −10.857 | −60.602 | 87.316 | 1.00 | 193.93 | C |
| ATOM | 4877 | C | LEU | A | 687 | −11.821 | −59.716 | 86.558 | 1.00 | 193.92 | C |
| ATOM | 4878 | O | LEU | A | 687 | −11.405 | −58.955 | 85.675 | 1.00 | 193.98 | O |
| ATOM | 4879 | CB | LEU | A | 687 | −10.107 | −59.801 | 88.411 | 1.00 | 194.15 | C |
| ATOM | 4880 | CG | LEU | A | 687 | −9.669 | −58.318 | 88.306 | 1.00 | 194.48 | C |
| ATOM | 4881 | CD1 | LEU | A | 687 | −10.780 | −57.341 | 88.735 | 1.00 | 194.50 | C |
| ATOM | 4882 | CD2 | LEU | A | 687 | −8.407 | −58.045 | 89.128 | 1.00 | 194.08 | C |
| ATOM | 4883 | N | TRP | A | 688 | −13.105 | −59.844 | 86.864 | 1.00 | 193.88 | N |
| ATOM | 4884 | CA | TRP | A | 688 | −14.088 | −58.922 | 86.320 | 1.00 | 193.97 | C |
| ATOM | 4885 | C | TRP | A | 688 | −15.026 | −58.509 | 87.428 | 1.00 | 194.14 | C |
| ATOM | 4886 | O | TRP | A | 688 | −15.585 | −59.354 | 88.121 | 1.00 | 194.05 | O |
| ATOM | 4887 | CB | TRP | A | 688 | −14.847 | −59.518 | 85.129 | 1.00 | 193.78 | C |
| ATOM | 4888 | CG | TRP | A | 688 | −13.966 | −59.900 | 83.958 | 1.00 | 193.64 | C |
| ATOM | 4889 | CD1 | TRP | A | 688 | −13.637 | −61.166 | 83.569 | 1.00 | 193.43 | C |
| ATOM | 4890 | CD2 | TRP | A | 688 | −13.303 | −59.014 | 83.035 | 1.00 | 193.54 | C |
| ATOM | 4891 | NE1 | TRP | A | 688 | −12.818 | −61.126 | 82.467 | 1.00 | 193.56 | N |
| ATOM | 4892 | CE2 | TRP | A | 688 | −12.596 | −59.820 | 82.119 | 1.00 | 193.55 | C |
| ATOM | 4893 | CE3 | TRP | A | 688 | −13.235 | −57.620 | 82.897 | 1.00 | 193.38 | C |
| ATOM | 4894 | CZ2 | TRP | A | 688 | −11.833 | −59.279 | 81.075 | 1.00 | 193.43 | C |
| ATOM | 4895 | CZ3 | TRP | A | 688 | −12.481 | −57.084 | 81.855 | 1.00 | 193.41 | C |
| ATOM | 4896 | CH2 | TRP | A | 688 | −11.792 | −57.913 | 80.960 | 1.00 | 193.37 | C |
| ATOM | 4897 | N | ILE | A | 689 | −15.161 | −57.198 | 87.598 | 1.00 | 194.55 | N |
| ATOM | 4898 | CA | ILE | A | 689 | −15.964 | −56.617 | 88.671 | 1.00 | 195.02 | C |
| ATOM | 4899 | C | ILE | A | 689 | −17.451 | −56.559 | 88.300 | 1.00 | 195.04 | C |
| ATOM | 4900 | O | ILE | A | 689 | −17.976 | −55.523 | 87.895 | 1.00 | 194.93 | O |
| ATOM | 4901 | CB | ILE | A | 689 | −15.375 | −55.230 | 89.185 | 1.00 | 195.18 | C |
| ATOM | 4902 | CG1 | ILE | A | 689 | −16.213 | −54.655 | 90.354 | 1.00 | 195.52 | C |
| ATOM | 4903 | CG2 | ILE | A | 689 | −15.157 | −54.222 | 88.016 | 1.00 | 195.30 | C |
| ATOM | 4904 | CD1 | ILE | A | 689 | −15.466 | −53.692 | 91.313 | 1.00 | 195.18 | C |
| ATOM | 4905 | N | LEU | A | 690 | −18.117 | −57.701 | 88.425 | 1.00 | 195.42 | N |
| ATOM | 4906 | CA | LEU | A | 690 | −19.566 | −57.745 | 88.316 | 1.00 | 195.92 | C |
| ATOM | 4907 | C | LEU | A | 690 | −20.144 | −56.870 | 89.400 | 1.00 | 196.44 | C |
| ATOM | 4908 | O | LEU | A | 690 | −19.759 | −56.964 | 90.564 | 1.00 | 196.58 | O |
| ATOM | 4909 | CB | LEU | A | 690 | −20.105 | −59.178 | 88.451 | 1.00 | 195.81 | C |
| ATOM | 4910 | CG | LEU | A | 690 | −21.561 | −59.523 | 88.843 | 1.00 | 195.48 | C |
| ATOM | 4911 | CD1 | LEU | A | 690 | −21.760 | −59.532 | 90.358 | 1.00 | 195.52 | C |
| ATOM | 4912 | CD2 | LEU | A | 690 | −22.635 | −58.659 | 88.163 | 1.00 | 195.26 | C |
| ATOM | 4913 | N | GLY | A | 691 | −21.069 | −56.015 | 89.000 | 1.00 | 197.05 | N |
| ATOM | 4914 | CA | GLY | A | 691 | −21.819 | −55.212 | 89.934 | 1.00 | 197.86 | C |
| ATOM | 4915 | C | GLY | A | 691 | −23.030 | −54.731 | 89.195 | 1.00 | 198.39 | C |
| ATOM | 4916 | O | GLY | A | 691 | −23.714 | −55.512 | 88.541 | 1.00 | 198.25 | O |
| ATOM | 4917 | N | CYS | A | 692 | −23.280 | −53.436 | 89.285 | 1.00 | 199.21 | N |
| ATOM | 4918 | CA | CYS | A | 692 | −24.329 | −52.839 | 88.505 | 1.00 | 200.20 | C |
| ATOM | 4919 | C | CYS | A | 692 | −23.909 | −51.472 | 88.002 | 1.00 | 201.25 | C |
| ATOM | 4920 | O | CYS | A | 692 | −23.142 | −50.756 | 88.649 | 1.00 | 201.12 | O |
| ATOM | 4921 | CB | CYS | A | 692 | −25.635 | −52.781 | 89.289 | 1.00 | 199.92 | C |
| ATOM | 4922 | SG | CYS | A | 692 | −25.684 | −51.507 | 90.504 | 1.00 | 199.43 | S |
| ATOM | 4923 | N | HIS | A | 693 | −24.441 | −51.144 | 86.827 | 1.00 | 202.81 | N |
| ATOM | 4924 | CA | HIS | A | 693 | −24.155 | −49.928 | 86.057 | 1.00 | 204.22 | C |
| ATOM | 4925 | C | HIS | A | 693 | −24.519 | −48.647 | 86.818 | 1.00 | 204.73 | C |
| ATOM | 4926 | O | HIS | A | 693 | −24.290 | −47.529 | 86.332 | 1.00 | 204.78 | O |
| ATOM | 4927 | CB | HIS | A | 693 | −24.942 | −49.990 | 84.740 | 1.00 | 204.54 | C |
| ATOM | 4928 | CG | HIS | A | 693 | −24.206 | −49.453 | 83.550 | 1.00 | 206.09 | C |
| ATOM | 4929 | ND1 | HIS | A | 693 | −24.267 | −48.128 | 83.169 | 1.00 | 207.51 | N |
| ATOM | 4930 | CD2 | HIS | A | 693 | −23.415 | −50.069 | 82.637 | 1.00 | 207.48 | C |
| ATOM | 4931 | CE1 | HIS | A | 693 | −23.536 | −47.949 | 82.081 | 1.00 | 208.18 | C |
| ATOM | 4932 | NE2 | HIS | A | 693 | −23.009 | −49.112 | 81.736 | 1.00 | 208.42 | N |
| ATOM | 4933 | N | ASN | A | 694 | −25.103 | −48.823 | 88.001 | 1.00 | 205.44 | N |
| ATOM | 4934 | CA | ASN | A | 694 | −25.403 | −47.716 | 88.894 | 1.00 | 206.08 | C |
| ATOM | 4935 | C | ASN | A | 694 | −24.289 | −47.646 | 89.926 | 1.00 | 206.13 | C |
| ATOM | 4936 | O | ASN | A | 694 | −24.051 | −48.613 | 90.646 | 1.00 | 206.23 | O |
| ATOM | 4937 | CB | ASN | A | 694 | −26.771 | −47.911 | 89.566 | 1.00 | 206.32 | C |
| ATOM | 4938 | CG | ASN | A | 694 | −27.879 | −48.294 | 88.569 | 1.00 | 207.34 | C |
| ATOM | 4939 | OD1 | ASN | A | 694 | −28.138 | −47.578 | 87.588 | 1.00 | 208.16 | O |
| ATOM | 4940 | ND2 | ASN | A | 694 | −28.541 | −49.428 | 88.828 | 1.00 | 208.17 | N |
| ATOM | 4941 | N | SER | A | 695 | −23.586 | −46.516 | 89.965 | 1.00 | 206.21 | N |
| ATOM | 4942 | CA | SER | A | 695 | −22.475 | −46.334 | 90.892 | 1.00 | 206.22 | C |
| ATOM | 4943 | C | SER | A | 695 | −22.895 | −45.523 | 92.119 | 1.00 | 206.27 | C |
| ATOM | 4944 | O | SER | A | 695 | −22.311 | −44.477 | 92.418 | 1.00 | 206.29 | O |
| ATOM | 4945 | CB | SER | A | 695 | −21.265 | −45.707 | 90.189 | 1.00 | 206.23 | C |
| ATOM | 4946 | OG | SER | A | 695 | −20.446 | −46.685 | 89.570 | 1.00 | 206.02 | O |
| ATOM | 4947 | N | ASP | A | 696 | −23.924 | −46.018 | 92.811 | 1.00 | 206.30 | N |
| ATOM | 4948 | CA | ASP | A | 696 | −24.366 | −45.478 | 94.107 | 1.00 | 206.28 | C |
| ATOM | 4949 | C | ASP | A | 696 | −24.858 | −46.580 | 95.046 | 1.00 | 205.81 | C |
| ATOM | 4950 | O | ASP | A | 696 | −25.273 | −46.302 | 96.171 | 1.00 | 205.70 | O |
| ATOM | 4951 | CB | ASP | A | 696 | −25.418 | −44.360 | 93.950 | 1.00 | 206.65 | C |
| ATOM | 4952 | CG | ASP | A | 696 | −26.408 | −44.623 | 92.815 | 1.00 | 207.59 | C |
| ATOM | 4953 | OD1 | ASP | A | 696 | −26.567 | −43.729 | 91.948 | 1.00 | 208.42 | O |
| ATOM | 4954 | OD2 | ASP | A | 696 | −27.021 | −45.716 | 92.787 | 1.00 | 208.34 | O |
| ATOM | 4955 | N | PHE | A | 697 | −24.807 | −47.823 | 94.563 | 1.00 | 205.37 | N |
| ATOM | 4956 | CA | PHE | A | 697 | −24.994 | −49.007 | 95.397 | 1.00 | 205.07 | C |
| ATOM | 4957 | C | PHE | A | 697 | −23.721 | −49.816 | 95.380 | 1.00 | 204.69 | C |
| ATOM | 4958 | O | PHE | A | 697 | −23.319 | −50.350 | 96.403 | 1.00 | 204.55 | O |
| ATOM | 4959 | CB | PHE | A | 697 | −26.135 | −49.910 | 94.910 | 1.00 | 205.31 | C |
| ATOM | 4960 | CG | PHE | A | 697 | −27.286 | −49.176 | 94.313 | 1.00 | 205.87 | C |
| ATOM | 4961 | CD1 | PHE | A | 697 | −28.044 | −48.292 | 95.078 | 1.00 | 206.64 | C |
| ATOM | 4962 | CD2 | PHE | A | 697 | −27.627 | −49.377 | 92.980 | 1.00 | 206.30 | C |
| ATOM | 4963 | CE1 | PHE | A | 697 | −29.116 | −47.601 | 94.516 | 1.00 | 207.10 | C |
| ATOM | 4964 | CE2 | PHE | A | 697 | −28.698 | −48.699 | 92.406 | 1.00 | 206.57 | C |
| ATOM | 4965 | CZ | PHE | A | 697 | −29.446 | −47.809 | 93.175 | 1.00 | 206.67 | C |
| ATOM | 4966 | N | ARG | A | 698 | −23.092 | −49.921 | 94.212 | 1.00 | 204.46 | N |
| ATOM | 4967 | CA | ARG | A | 698 | −21.838 | −50.675 | 94.082 | 1.00 | 204.33 | C |
| ATOM | 4968 | C | ARG | A | 698 | −20.708 | −50.086 | 94.940 | 1.00 | 204.27 | C |
| ATOM | 4969 | O | ARG | A | 698 | −19.554 | −50.510 | 94.846 | 1.00 | 204.52 | O |
| ATOM | 4970 | CB | ARG | A | 698 | −21.404 | −50.881 | 92.612 | 1.00 | 204.31 | C |
| ATOM | 4971 | CG | ARG | A | 698 | −21.674 | −49.734 | 91.658 | 1.00 | 204.30 | C |
| ATOM | 4972 | CD | ARG | A | 698 | −20.895 | −49.868 | 90.361 | 1.00 | 204.21 | C |
| ATOM | 4973 | NE | ARG | A | 698 | −19.733 | −48.988 | 90.359 | 1.00 | 204.58 | N |
| ATOM | 4974 | CZ | ARG | A | 698 | −18.532 | −49.313 | 90.832 | 1.00 | 205.10 | C |
| ATOM | 4975 | NH1 | ARG | A | 698 | −17.551 | −48.421 | 90.788 | 1.00 | 205.24 | N |
| ATOM | 4976 | NH2 | ARG | A | 698 | −18.304 | −50.520 | 91.348 | 1.00 | 205.04 | N |
| ATOM | 4977 | N | ASN | A | 699 | −21.063 | −49.111 | 95.775 | 1.00 | 203.96 | N |
| ATOM | 4978 | CA | ASN | A | 699 | −20.187 | −48.576 | 96.818 | 1.00 | 203.55 | C |
| ATOM | 4979 | C | ASN | A | 699 | −20.798 | −48.844 | 98.194 | 1.00 | 203.15 | C |
| ATOM | 4980 | O | ASN | A | 699 | −20.078 | −48.992 | 99.183 | 1.00 | 203.10 | O |
| ATOM | 4981 | CB | ASN | A | 699 | −19.939 | −47.078 | 96.609 | 1.00 | 203.55 | C |
| ATOM | 4982 | CG | ASN | A | 699 | −21.206 | −46.317 | 96.229 | 1.00 | 203.86 | C |
| ATOM | 4983 | OD1 | ASN | A | 699 | −22.315 | −46.857 | 96.276 | 1.00 | 204.22 | O |
| ATOM | 4984 | ND2 | ASN | A | 699 | −21.042 | −45.056 | 95.848 | 1.00 | 204.11 | N |
| ATOM | 4985 | N | ARG | A | 700 | −22.133 | −48.902 | 98.230 | 1.00 | 202.64 | N |
| ATOM | 4986 | CA | ARG | A | 700 | −22.906 | −49.334 | 99.403 | 1.00 | 202.08 | C |
| ATOM | 4987 | C | ARG | A | 700 | −22.884 | −50.879 | 99.507 | 1.00 | 201.87 | C |
| ATOM | 4988 | O | ARG | A | 700 | −23.770 | −51.494 | 100.126 | 1.00 | 201.82 | O |
| ATOM | 4989 | CB | ARG | A | 700 | −24.362 | −48.834 | 99.303 | 1.00 | 201.97 | C |
| ATOM | 4990 | CG | ARG | A | 700 | −24.561 | −47.315 | 99.244 | 1.00 | 201.33 | C |
| ATOM | 4991 | CD | ARG | A | 700 | −25.133 | −46.751 | 100.537 | 1.00 | 200.27 | C |
| ATOM | 4992 | NE | ARG | A | 700 | −25.558 | −45.357 | 100.404 | 1.00 | 200.23 | N |
| ATOM | 4993 | CZ | ARG | A | 700 | −24.754 | −44.295 | 100.508 | 1.00 | 200.45 | C |
| ATOM | 4994 | NH1 | ARG | A | 700 | −25.249 | −43.073 | 100.373 | 1.00 | 200.51 | N |
| ATOM | 4995 | NH2 | ARG | A | 700 | −23.456 | −44.437 | 100.744 | 1.00 | 200.51 | N |
| ATOM | 4996 | N | GLY | A | 701 | −21.861 | −51.489 | 98.904 | 1.00 | 201.48 | N |
| ATOM | 4997 | CA | GLY | A | 701 | −21.758 | −52.944 | 98.784 | 1.00 | 200.93 | C |
| ATOM | 4998 | C | GLY | A | 701 | −22.067 | −53.332 | 97.357 | 1.00 | 200.47 | C |
| ATOM | 4999 | O | GLY | A | 701 | −21.437 | −52.821 | 96.433 | 1.00 | 200.53 | O |
| ATOM | 5000 | N | MET | A | 702 | −23.028 | −54.243 | 97.188 | 1.00 | 199.87 | N |
| ATOM | 5001 | CA | MET | A | 702 | −23.681 | −54.496 | 95.897 | 1.00 | 199.18 | C |
| ATOM | 5002 | C | MET | A | 702 | −22.724 | −54.867 | 94.751 | 1.00 | 198.87 | C |
| ATOM | 5003 | O | MET | A | 702 | −23.033 | −54.623 | 93.588 | 1.00 | 198.95 | O |
| ATOM | 5004 | CB | MET | A | 702 | −24.559 | −53.286 | 95.520 | 1.00 | 199.10 | C |
| ATOM | 5005 | CG | MET | A | 702 | −25.596 | −53.500 | 94.422 | 1.00 | 198.97 | C |
| ATOM | 5006 | SD | MET | A | 702 | −26.859 | −54.714 | 94.821 | 1.00 | 199.01 | S |
| ATOM | 5007 | CE | MET | A | 702 | −28.204 | −54.156 | 93.787 | 1.00 | 198.60 | C |
| ATOM | 5008 | N | THR | A | 703 | −21.563 | −55.440 | 95.072 | 1.00 | 198.40 | N |
| ATOM | 5009 | CA | THR | A | 703 | −20.661 | −55.969 | 94.040 | 1.00 | 197.90 | C |
| ATOM | 5010 | C | THR | A | 703 | −20.118 | −57.315 | 94.424 | 1.00 | 197.45 | C |
| ATOM | 5011 | O | THR | A | 703 | −19.903 | −57.602 | 95.597 | 1.00 | 197.28 | O |
| ATOM | 5012 | CB | THR | A | 703 | −19.428 | −55.073 | 93.749 | 1.00 | 198.02 | C |
| ATOM | 5013 | OG1 | THR | A | 703 | −18.768 | −54.731 | 94.976 | 1.00 | 198.46 | O |
| ATOM | 5014 | CG2 | THR | A | 703 | −19.803 | −53.814 | 92.962 | 1.00 | 197.84 | C |
| ATOM | 5015 | N | ALA | A | 704 | −19.892 | −58.135 | 93.415 | 1.00 | 197.15 | N |
| ATOM | 5016 | CA | ALA | A | 704 | −19.154 | −59.357 | 93.592 | 1.00 | 197.23 | C |
| ATOM | 5017 | C | ALA | A | 704 | −18.029 | −59.371 | 92.562 | 1.00 | 197.27 | C |
| ATOM | 5018 | O | ALA | A | 704 | −17.848 | −58.391 | 91.846 | 1.00 | 197.30 | O |
| ATOM | 5019 | CB | ALA | A | 704 | −20.071 | −60.536 | 93.425 | 1.00 | 197.30 | C |
| ATOM | 5020 | N | LEU | A | 705 | −17.265 | −60.463 | 92.509 | 1.00 | 197.35 | N |
| ATOM | 5021 | CA | LEU | A | 705 | −16.226 | −60.658 | 91.488 | 1.00 | 197.42 | C |
| ATOM | 5022 | C | LEU | A | 705 | −16.487 | −61.902 | 90.644 | 1.00 | 197.41 | C |
| ATOM | 5023 | O | LEU | A | 705 | −17.263 | −62.765 | 91.032 | 1.00 | 197.46 | O |
| ATOM | 5024 | CB | LEU | A | 705 | −14.856 | −60.789 | 92.147 | 1.00 | 197.40 | C |
| ATOM | 5025 | CG | LEU | A | 705 | −13.938 | −59.574 | 92.114 | 1.00 | 197.76 | C |
| ATOM | 5026 | CD1 | LEU | A | 705 | −12.953 | −59.683 | 93.263 | 1.00 | 198.23 | C |
| ATOM | 5027 | CD2 | LEU | A | 705 | −13.220 | −59.443 | 90.755 | 1.00 | 197.83 | C |
| ATOM | 5028 | N | LEU | A | 706 | −15.848 | −62.003 | 89.485 | 1.00 | 197.42 | N |
| ATOM | 5029 | CA | LEU | A | 706 | −15.865 | −63.271 | 88.766 | 1.00 | 197.53 | C |
| ATOM | 5030 | C | LEU | A | 706 | −14.479 | −63.600 | 88.232 | 1.00 | 197.81 | C |
| ATOM | 5031 | O | LEU | A | 706 | −13.712 | −62.701 | 87.844 | 1.00 | 197.89 | O |
| ATOM | 5032 | CB | LEU | A | 706 | −16.938 | −63.301 | 87.672 | 1.00 | 197.33 | C |
| ATOM | 5033 | CG | LEU | A | 706 | −16.732 | −62.487 | 86.397 | 1.00 | 197.22 | C |
| ATOM | 5034 | CD1 | LEU | A | 706 | −16.203 | −63.379 | 85.286 | 1.00 | 197.23 | C |
| ATOM | 5035 | CD2 | LEU | A | 706 | −18.030 | −61.839 | 85.969 | 1.00 | 196.79 | C |
| ATOM | 5036 | N | LYS | A | 707 | −14.161 | −64.892 | 88.248 | 1.00 | 198.01 | N |
| ATOM | 5037 | CA | LYS | A | 707 | −12.851 | −65.375 | 87.823 | 1.00 | 198.18 | C |
| ATOM | 5038 | C | LYS | A | 707 | −12.968 | −66.317 | 86.633 | 1.00 | 198.11 | C |
| ATOM | 5039 | O | LYS | A | 707 | −13.954 | −67.047 | 86.499 | 1.00 | 198.21 | O |
| ATOM | 5040 | CB | LYS | A | 707 | −12.078 | −66.028 | 88.986 | 1.00 | 198.24 | C |
| ATOM | 5041 | CG | LYS | A | 707 | −12.862 | −67.023 | 89.870 | 1.00 | 198.39 | C |
| ATOM | 5042 | CD | LYS | A | 707 | −11.966 | −67.568 | 91.002 | 1.00 | 198.37 | C |
| ATOM | 5043 | CE | LYS | A | 707 | −12.738 | −68.406 | 92.026 | 1.00 | 198.18 | C |
| ATOM | 5044 | NZ | LYS | A | 707 | −11.854 | −69.002 | 93.075 | 1.00 | 197.47 | N |
| ATOM | 5045 | N | VAL | A | 708 | −11.963 | −66.277 | 85.763 | 1.00 | 197.91 | N |
| ATOM | 5046 | CA | VAL | A | 708 | −11.970 | −67.062 | 84.542 | 1.00 | 197.70 | C |
| ATOM | 5047 | C | VAL | A | 708 | −10.547 | −67.495 | 84.229 | 1.00 | 197.71 | C |
| ATOM | 5048 | O | VAL | A | 708 | −9.631 | −66.676 | 84.270 | 1.00 | 197.71 | O |
| ATOM | 5049 | CB | VAL | A | 708 | −12.513 | −66.245 | 83.342 | 1.00 | 197.69 | C |
| ATOM | 5050 | CG1 | VAL | A | 708 | −13.431 | −67.099 | 82.509 | 1.00 | 197.74 | C |
| ATOM | 5051 | CG2 | VAL | A | 708 | −13.256 | −64.989 | 83.797 | 1.00 | 197.50 | C |
| ATOM | 5052 | N | SER | A | 709 | −10.372 | −68.783 | 83.929 | 1.00 | 197.82 | N |
| ATOM | 5053 | CA | SER | A | 709 | −9.081 | −69.367 | 83.505 | 1.00 | 197.96 | C |
| ATOM | 5054 | C | SER | A | 709 | −9.313 | −70.781 | 82.981 | 1.00 | 197.95 | C |
| ATOM | 5055 | O | SER | A | 709 | −10.236 | −71.447 | 83.426 | 1.00 | 197.99 | O |
| ATOM | 5056 | CB | SER | A | 709 | −8.086 | −69.411 | 84.668 | 1.00 | 198.00 | C |
| ATOM | 5057 | OG | SER | A | 709 | −8.692 | −69.949 | 85.835 | 1.00 | 198.21 | O |
| ATOM | 5058 | N | SER | A | 710 | −8.475 | −71.248 | 82.058 | 1.00 | 197.97 | N |
| ATOM | 5059 | CA | SER | A | 710 | −8.672 | −72.568 | 81.424 | 1.00 | 198.08 | C |
| ATOM | 5060 | C | SER | A | 710 | −8.928 | −73.750 | 82.408 | 1.00 | 198.26 | C |
| ATOM | 5061 | O | SER | A | 710 | −8.518 | −73.709 | 83.573 | 1.00 | 198.13 | O |
| ATOM | 5062 | CB | SER | A | 710 | −7.515 | −72.876 | 80.455 | 1.00 | 198.11 | C |
| ATOM | 5063 | OG | SER | A | 710 | −7.320 | −71.829 | 79.511 | 1.00 | 197.53 | O |
| ATOM | 5064 | N | CYS | A | 711 | −9.608 | −74.793 | 81.921 | 1.00 | 198.52 | N |
| ATOM | 5065 | CA | CYS | A | 711 | −10.077 | −75.914 | 82.754 | 1.00 | 198.91 | C |
| ATOM | 5066 | C | CYS | A | 711 | −9.394 | −77.240 | 82.429 | 1.00 | 198.88 | C |
| ATOM | 5067 | O | CYS | A | 711 | −8.167 | −77.297 | 82.373 | 1.00 | 198.87 | O |
| ATOM | 5068 | CB | CYS | A | 711 | −11.593 | −76.067 | 82.617 | 1.00 | 199.07 | C |
| ATOM | 5069 | SG | CYS | A | 711 | −12.552 | −75.148 | 83.844 | 1.00 | 200.34 | S |
| ATOM | 5070 | N | ASP | A | 712 | −10.192 | −78.300 | 82.242 | 1.00 | 199.02 | N |
| ATOM | 5071 | CA | ASP | A | 712 | −9.691 | −79.616 | 81.781 | 1.00 | 199.19 | C |
| ATOM | 5072 | C | ASP | A | 712 | −10.717 | −80.619 | 81.201 | 1.00 | 199.30 | C |
| ATOM | 5073 | O | ASP | A | 712 | −10.625 | −80.961 | 80.018 | 1.00 | 199.46 | O |
| ATOM | 5074 | CB | ASP | A | 712 | −8.808 | −80.301 | 82.839 | 1.00 | 199.18 | C |
| ATOM | 5075 | CG | ASP | A | 712 | −7.335 | −80.287 | 82.463 | 1.00 | 198.90 | C |
| ATOM | 5076 | OD1 | ASP | A | 712 | −6.527 | −79.766 | 83.256 | 1.00 | 198.74 | O |
| ATOM | 5077 | OD2 | ASP | A | 712 | −6.989 | −80.785 | 81.369 | 1.00 | 198.36 | O |
| ATOM | 5078 | N | LYS | A | 713 | −11.658 | −81.099 | 82.025 | 1.00 | 199.26 | N |
| ATOM | 5079 | CA | LYS | A | 713 | −12.614 | −82.181 | 81.656 | 1.00 | 199.04 | C |
| ATOM | 5080 | C | LYS | A | 713 | −11.972 | −83.562 | 81.394 | 1.00 | 198.97 | C |
| ATOM | 5081 | O | LYS | A | 713 | −10.787 | −83.795 | 81.652 | 1.00 | 198.72 | O |
| ATOM | 5082 | CB | LYS | A | 713 | −13.510 | −81.791 | 80.459 | 1.00 | 198.97 | C |
| ATOM | 5083 | CG | LYS | A | 713 | −14.523 | −80.684 | 80.707 | 1.00 | 198.37 | C |
| ATOM | 5084 | CD | LYS | A | 713 | −15.815 | −81.202 | 81.304 | 1.00 | 197.74 | C |
| ATOM | 5085 | CE | LYS | A | 713 | −15.790 | −81.122 | 82.815 | 1.00 | 197.50 | C |
| ATOM | 5086 | NZ | LYS | A | 713 | −17.135 | −80.760 | 83.319 | 1.00 | 197.88 | N |
| TER | 5087 | | LYS | A | 713 | | | | | | |
| ATOM | 5088 | N | PHE | B | 1691 | −11.473 | −60.640 | 49.225 | 1.00 | 219.51 | N |
| ATOM | 5089 | CA | PHE | B | 1691 | −11.829 | −59.653 | 50.290 | 1.00 | 219.65 | C |
| ATOM | 5090 | C | PHE | B | 1691 | −10.936 | −59.830 | 51.517 | 1.00 | 219.45 | C |
| ATOM | 5091 | O | PHE | B | 1691 | −10.571 | −60.953 | 51.863 | 1.00 | 219.42 | O |
| ATOM | 5092 | CB | PHE | B | 1691 | −13.316 | −59.757 | 50.672 | 1.00 | 219.86 | C |
| ATOM | 5093 | CG | PHE | B | 1691 | −14.261 | −59.123 | 49.661 | 1.00 | 220.52 | C |
| ATOM | 5094 | CD1 | PHE | B | 1691 | −14.810 | −59.882 | 48.619 | 1.00 | 220.83 | C |
| ATOM | 5095 | CD2 | PHE | B | 1691 | −14.608 | −57.771 | 49.756 | 1.00 | 220.82 | C |
| ATOM | 5096 | CE1 | PHE | B | 1691 | −15.687 | −59.302 | 47.683 | 1.00 | 220.51 | C |
| ATOM | 5097 | CE2 | PHE | B | 1691 | −15.481 | −57.181 | 48.823 | 1.00 | 220.58 | C |
| ATOM | 5098 | CZ | PHE | B | 1691 | −16.021 | −57.950 | 47.789 | 1.00 | 220.38 | C |
| ATOM | 5099 | N | GLN | B | 1692 | −10.610 | −58.717 | 52.173 | 1.00 | 219.27 | N |
| ATOM | 5100 | CA | GLN | B | 1692 | −9.568 | −58.669 | 53.212 | 1.00 | 219.14 | C |
| ATOM | 5101 | C | GLN | B | 1692 | −10.059 | −58.364 | 54.647 | 1.00 | 219.08 | C |
| ATOM | 5102 | O | GLN | B | 1692 | −11.229 | −58.017 | 54.851 | 1.00 | 219.27 | O |
| ATOM | 5103 | CB | GLN | B | 1692 | −8.510 | −57.633 | 52.812 | 1.00 | 219.10 | C |
| ATOM | 5104 | CG | GLN | B | 1692 | −7.641 | −58.038 | 51.640 | 1.00 | 219.27 | C |
| ATOM | 5105 | CD | GLN | B | 1692 | −6.750 | −59.221 | 51.965 | 1.00 | 219.82 | C |
| ATOM | 5106 | OE1 | GLN | B | 1692 | −6.623 | −59.624 | 53.128 | 1.00 | 219.51 | O |
| ATOM | 5107 | NE2 | GLN | B | 1692 | −6.127 | −59.789 | 50.933 | 1.00 | 220.34 | N |
| ATOM | 5108 | N | LYS | B | 1693 | −9.156 | −58.512 | 55.629 | 1.00 | 218.78 | N |
| ATOM | 5109 | CA | LYS | B | 1693 | −9.356 | −58.020 | 57.016 | 1.00 | 218.33 | C |
| ATOM | 5110 | C | LYS | B | 1693 | −8.476 | −56.776 | 57.235 | 1.00 | 217.89 | C |
| ATOM | 5111 | O | LYS | B | 1693 | −7.408 | −56.663 | 56.619 | 1.00 | 217.98 | O |
| ATOM | 5112 | CB | LYS | B | 1693 | −8.962 | −59.075 | 58.065 | 1.00 | 218.40 | C |
| ATOM | 5113 | CG | LYS | B | 1693 | −9.712 | −60.407 | 58.038 | 1.00 | 218.51 | C |
| ATOM | 5114 | CD | LYS | B | 1693 | −9.193 | −61.335 | 59.149 | 1.00 | 218.40 | C |
| ATOM | 5115 | CE | LYS | B | 1693 | −9.705 | −62.767 | 58.999 | 1.00 | 218.31 | C |
| ATOM | 5116 | NZ | LYS | B | 1693 | −9.525 | −63.565 | 60.251 | 1.00 | 217.97 | N |
| ATOM | 5117 | N | LYS | B | 1694 | −8.894 | −55.860 | 58.113 | 1.00 | 217.20 | N |
| ATOM | 5118 | CA | LYS | B | 1694 | −8.069 | −54.674 | 58.403 | 1.00 | 216.45 | C |
| ATOM | 5119 | C | LYS | B | 1694 | −7.768 | −54.433 | 59.888 | 1.00 | 215.95 | C |
| ATOM | 5120 | O | LYS | B | 1694 | −8.631 | −54.583 | 60.754 | 1.00 | 215.91 | O |
| ATOM | 5121 | CB | LYS | B | 1694 | −8.631 | −53.400 | 57.744 | 1.00 | 216.43 | C |
| ATOM | 5122 | CG | LYS | B | 1694 | −7.537 | −52.410 | 57.329 | 1.00 | 216.33 | C |
| ATOM | 5123 | CD | LYS | B | 1694 | −8.064 | −51.044 | 56.918 | 1.00 | 216.37 | C |
| ATOM | 5124 | CE | LYS | B | 1694 | −6.908 | −50.050 | 56.781 | 1.00 | 216.47 | C |
| ATOM | 5125 | NZ | LYS | B | 1694 | −7.356 | −48.649 | 56.551 | 1.00 | 216.35 | N |
| ATOM | 5126 | N | THR | B | 1695 | −6.517 | −54.072 | 60.153 | 1.00 | 215.29 | N |
| ATOM | 5127 | CA | THR | B | 1695 | −6.075 | −53.641 | 61.464 | 1.00 | 214.61 | C |
| ATOM | 5128 | C | THR | B | 1695 | −6.099 | −52.107 | 61.510 | 1.00 | 214.30 | C |
| ATOM | 5129 | O | THR | B | 1695 | −5.165 | −51.445 | 61.059 | 1.00 | 214.24 | O |
| ATOM | 5130 | CB | THR | B | 1695 | −4.693 | −54.262 | 61.826 | 1.00 | 214.56 | C |
| ATOM | 5131 | OG1 | THR | B | 1695 | −4.080 | −53.503 | 62.869 | 1.00 | 214.52 | O |
| ATOM | 5132 | CG2 | THR | B | 1695 | −3.749 | −54.338 | 60.609 | 1.00 | 214.50 | C |
| ATOM | 5133 | N | ARG | B | 1696 | −7.196 | −51.559 | 62.034 | 1.00 | 213.96 | N |
| ATOM | 5134 | CA | ARG | B | 1696 | −7.476 | −50.118 | 62.006 | 1.00 | 213.69 | C |
| ATOM | 5135 | C | ARG | B | 1696 | −7.156 | −49.454 | 63.327 | 1.00 | 213.54 | C |
| ATOM | 5136 | O | ARG | B | 1696 | −7.942 | −49.549 | 64.259 | 1.00 | 213.43 | O |
| ATOM | 5137 | CB | ARG | B | 1696 | −8.956 | −49.882 | 61.700 | 1.00 | 213.59 | C |
| ATOM | 5138 | CG | ARG | B | 1696 | −9.327 | −50.109 | 60.261 | 1.00 | 213.84 | C |
| ATOM | 5139 | CD | ARG | B | 1696 | −10.755 | −50.607 | 60.100 | 1.00 | 213.82 | C |
| ATOM | 5140 | NE | ARG | B | 1696 | −11.091 | −50.779 | 58.683 | 1.00 | 214.04 | N |
| ATOM | 5141 | CZ | ARG | B | 1696 | −12.183 | −51.388 | 58.224 | 1.00 | 214.00 | C |
| ATOM | 5142 | NH1 | ARG | B | 1696 | −13.065 | −51.905 | 59.070 | 1.00 | 214.35 | N |
| ATOM | 5143 | NH2 | ARG | B | 1696 | −12.390 | −51.486 | 56.914 | 1.00 | 213.43 | N |
| ATOM | 5144 | N | HIS | B | 1697 | −6.016 | −48.776 | 63.418 | 1.00 | 213.57 | N |
| ATOM | 5145 | CA | HIS | B | 1697 | −5.670 | −48.087 | 64.660 | 1.00 | 213.73 | C |
| ATOM | 5146 | C | HIS | B | 1697 | −6.400 | −46.762 | 64.709 | 1.00 | 213.59 | C |
| ATOM | 5147 | O | HIS | B | 1697 | −6.859 | −46.263 | 63.685 | 1.00 | 213.64 | O |
| ATOM | 5148 | CB | HIS | B | 1697 | −4.162 | −47.838 | 64.833 | 1.00 | 213.85 | C |
| ATOM | 5149 | CG | HIS | B | 1697 | −3.303 | −48.463 | 63.779 | 1.00 | 215.03 | C |
| ATOM | 5150 | ND1 | HIS | B | 1697 | −2.555 | −47.714 | 62.896 | 1.00 | 216.25 | N |
| ATOM | 5151 | CD2 | HIS | B | 1697 | −3.062 | −49.760 | 63.470 | 1.00 | 216.00 | C |
| ATOM | 5152 | CE1 | HIS | B | 1697 | −1.895 | −48.522 | 62.083 | 1.00 | 216.51 | C |
| ATOM | 5153 | NE2 | HIS | B | 1697 | −2.185 | −49.769 | 62.409 | 1.00 | 216.49 | N |
| ATOM | 5154 | N | TYR | B | 1698 | −6.514 | −46.218 | 65.918 | 1.00 | 213.56 | N |
| ATOM | 5155 | CA | TYR | B | 1698 | −7.056 | −44.886 | 66.169 | 1.00 | 213.42 | C |
| ATOM | 5156 | C | TYR | B | 1698 | −6.149 | −44.205 | 67.199 | 1.00 | 213.47 | C |
| ATOM | 5157 | O | TYR | B | 1698 | −6.115 | −44.596 | 68.372 | 1.00 | 213.57 | O |
| ATOM | 5158 | CB | TYR | B | 1698 | −8.492 | −44.968 | 66.703 | 1.00 | 213.22 | C |
| ATOM | 5159 | CG | TYR | B | 1698 | −9.550 | −45.329 | 65.678 | 1.00 | 212.97 | C |
| ATOM | 5160 | CD1 | TYR | B | 1698 | −9.744 | −46.649 | 65.278 | 1.00 | 212.82 | C |
| ATOM | 5161 | CD2 | TYR | B | 1698 | −10.378 | −44.351 | 65.128 | 1.00 | 213.18 | C |
| ATOM | 5162 | CE1 | TYR | B | 1698 | −10.726 | −46.986 | 64.341 | 1.00 | 212.83 | C |
| ATOM | 5163 | CE2 | TYR | B | 1698 | −11.367 | −44.679 | 64.191 | 1.00 | 213.17 | C |
| ATOM | 5164 | CZ | TYR | B | 1698 | −11.535 | −46.000 | 63.806 | 1.00 | 212.86 | C |
| ATOM | 5165 | OH | TYR | B | 1698 | −12.500 | −46.334 | 62.882 | 1.00 | 212.56 | O |
| ATOM | 5166 | N | PHE | B | 1699 | −5.400 | −43.204 | 66.754 | 1.00 | 213.36 | N |
| ATOM | 5167 | CA | PHE | B | 1699 | −4.497 | −42.486 | 67.637 | 1.00 | 213.28 | C |
| ATOM | 5168 | C | PHE | B | 1699 | −5.295 | −41.420 | 68.362 | 1.00 | 213.29 | C |
| ATOM | 5169 | O | PHE | B | 1699 | −5.699 | −40.418 | 67.769 | 1.00 | 213.29 | O |
| ATOM | 5170 | CB | PHE | B | 1699 | −3.340 | −41.903 | 66.840 | 1.00 | 213.28 | C |
| ATOM | 5171 | CG | PHE | B | 1699 | −2.481 | −42.946 | 66.190 | 1.00 | 213.43 | C |
| ATOM | 5172 | CD1 | PHE | B | 1699 | −3.019 | −43.830 | 65.256 | 1.00 | 213.69 | C |
| ATOM | 5173 | CD2 | PHE | B | 1699 | −1.137 | −43.054 | 66.515 | 1.00 | 213.69 | C |
| ATOM | 5174 | CE1 | PHE | B | 1699 | −2.228 | −44.803 | 64.654 | 1.00 | 213.95 | C |
| ATOM | 5175 | CE2 | PHE | B | 1699 | −0.328 | −44.016 | 65.912 | 1.00 | 213.94 | C |
| ATOM | 5176 | CZ | PHE | B | 1699 | −0.876 | −44.896 | 64.982 | 1.00 | 213.93 | C |
| ATOM | 5177 | N | ILE | B | 1700 | −5.524 | −41.665 | 69.651 | 1.00 | 213.29 | N |
| ATOM | 5178 | CA | ILE | B | 1700 | −6.520 | −40.942 | 70.444 | 1.00 | 213.21 | C |
| ATOM | 5179 | C | ILE | B | 1700 | −5.982 | −40.573 | 71.835 | 1.00 | 213.54 | C |
| ATOM | 5180 | O | ILE | B | 1700 | −5.097 | −41.247 | 72.364 | 1.00 | 213.54 | O |
| ATOM | 5181 | CB | ILE | B | 1700 | −7.838 | −41.768 | 70.533 | 1.00 | 212.92 | C |
| ATOM | 5182 | CG1 | ILE | B | 1700 | −8.873 | −41.088 | 71.429 | 1.00 | 212.49 | C |
| ATOM | 5183 | CG2 | ILE | B | 1700 | −7.556 | −43.187 | 70.991 | 1.00 | 212.57 | C |
| ATOM | 5184 | CD1 | ILE | B | 1700 | −10.252 | −41.676 | 71.298 | 1.00 | 212.15 | C |
| ATOM | 5185 | N | ALA | B | 1701 | −6.511 | −39.492 | 72.409 | 1.00 | 213.94 | N |
| ATOM | 5186 | CA | ALA | B | 1701 | −6.048 | −38.985 | 73.705 | 1.00 | 214.28 | C |
| ATOM | 5187 | C | ALA | B | 1701 | −7.181 | −38.375 | 74.532 | 1.00 | 214.50 | C |
| ATOM | 5188 | O | ALA | B | 1701 | −8.354 | −38.467 | 74.154 | 1.00 | 214.37 | O |
| ATOM | 5189 | CB | ALA | B | 1701 | −4.921 | −37.970 | 73.504 | 1.00 | 214.30 | C |
| ATOM | 5190 | N | ALA | B | 1702 | −6.817 | −37.763 | 75.660 | 1.00 | 214.86 | N |
| ATOM | 5191 | CA | ALA | B | 1702 | −7.779 | −37.109 | 76.538 | 1.00 | 215.38 | C |
| ATOM | 5192 | C | ALA | B | 1702 | −7.275 | −35.739 | 76.971 | 1.00 | 215.83 | C |
| ATOM | 5193 | O | ALA | B | 1702 | −6.196 | −35.640 | 77.553 | 1.00 | 215.80 | O |
| ATOM | 5194 | CB | ALA | B | 1702 | −8.059 | −37.977 | 77.745 | 1.00 | 215.32 | C |
| ATOM | 5195 | N | VAL | B | 1703 | −8.056 | −34.694 | 76.675 | 1.00 | 216.52 | N |
| ATOM | 5196 | CA | VAL | B | 1703 | −7.682 | −33.287 | 76.950 | 1.00 | 217.42 | C |
| ATOM | 5197 | C | VAL | B | 1703 | −8.881 | −32.480 | 77.523 | 1.00 | 217.58 | C |
| ATOM | 5198 | O | VAL | B | 1703 | −9.902 | −33.077 | 77.872 | 1.00 | 217.47 | O |
| ATOM | 5199 | CB | VAL | B | 1703 | −7.058 | −32.561 | 75.686 | 1.00 | 217.42 | C |
| ATOM | 5200 | CG1 | VAL | B | 1703 | −5.971 | −31.559 | 76.102 | 1.00 | 217.45 | C |
| ATOM | 5201 | CG2 | VAL | B | 1703 | −6.477 | −33.552 | 74.670 | 1.00 | 217.56 | C |
| ATOM | 5202 | N | GLU | B | 1704 | −8.747 | −31.145 | 77.632 | 1.00 | 218.08 | N |
| ATOM | 5203 | CA | GLU | B | 1704 | −9.806 | −30.254 | 78.178 | 1.00 | 218.38 | C |
| ATOM | 5204 | C | GLU | B | 1704 | −9.856 | −28.817 | 77.598 | 1.00 | 218.96 | C |
| ATOM | 5205 | O | GLU | B | 1704 | −8.867 | −28.313 | 77.052 | 1.00 | 218.86 | O |
| ATOM | 5206 | CB | GLU | B | 1704 | −9.722 | −30.145 | 79.710 | 1.00 | 218.48 | C |
| ATOM | 5207 | CG | GLU | B | 1704 | −8.832 | −31.153 | 80.429 | 1.00 | 218.59 | C |
| ATOM | 5208 | CD | GLU | B | 1704 | −7.402 | −30.662 | 80.581 | 1.00 | 218.80 | C |
| ATOM | 5209 | OE1 | GLU | B | 1704 | −6.752 | −30.385 | 79.547 | 1.00 | 218.79 | O |
| ATOM | 5210 | OE2 | GLU | B | 1704 | −6.928 | −30.554 | 81.736 | 1.00 | 218.80 | O |
| ATOM | 5211 | N | ARG | B | 1705 | −11.021 | −28.175 | 77.756 | 1.00 | 219.65 | N |
| ATOM | 5212 | CA | ARG | B | 1705 | −11.301 | −26.781 | 77.352 | 1.00 | 220.47 | C |
| ATOM | 5213 | C | ARG | B | 1705 | −12.656 | −26.375 | 77.945 | 1.00 | 220.89 | C |
| ATOM | 5214 | O | ARG | B | 1705 | −12.722 | −25.654 | 78.939 | 1.00 | 220.70 | O |
| ATOM | 5215 | CB | ARG | B | 1705 | −11.321 | −26.631 | 75.821 | 1.00 | 220.57 | C |
| ATOM | 5216 | CG | ARG | B | 1705 | −11.911 | −27.823 | 75.047 | 1.00 | 221.08 | C |
| ATOM | 5217 | CD | ARG | B | 1705 | −12.769 | −27.360 | 73.879 | 1.00 | 222.34 | C |
| ATOM | 5218 | NE | ARG | B | 1705 | −12.962 | −28.394 | 72.854 | 1.00 | 223.71 | N |
| ATOM | 5219 | CZ | ARG | B | 1705 | −13.944 | −29.302 | 72.844 | 1.00 | 224.27 | C |
| ATOM | 5220 | NH1 | ARG | B | 1705 | −14.847 | −29.337 | 73.822 | 1.00 | 224.26 | N |
| ATOM | 5221 | NH2 | ARG | B | 1705 | −14.021 | −30.185 | 71.847 | 1.00 | 224.09 | N |
| ATOM | 5222 | N | LEU | B | 1706 | −13.714 | −26.780 | 77.239 | 1.00 | 221.84 | N |
| ATOM | 5223 | CA | LEU | B | 1706 | −15.017 | −27.257 | 77.775 | 1.00 | 222.53 | C |
| ATOM | 5224 | C | LEU | B | 1706 | −16.347 | −26.726 | 77.227 | 1.00 | 223.01 | C |
| ATOM | 5225 | O | LEU | B | 1706 | −16.818 | −27.167 | 76.172 | 1.00 | 223.00 | O |
| ATOM | 5226 | CB | LEU | B | 1706 | −15.080 | −27.402 | 79.311 | 1.00 | 222.60 | C |
| ATOM | 5227 | CG | LEU | B | 1706 | −16.342 | −28.178 | 79.773 | 1.00 | 222.49 | C |
| ATOM | 5228 | CD1 | LEU | B | 1706 | −16.357 | −29.659 | 79.381 | 1.00 | 223.04 | C |
| ATOM | 5229 | CD2 | LEU | B | 1706 | −16.615 | −28.048 | 81.244 | 1.00 | 223.33 | C |
| ATOM | 5230 | N | TRP | B | 1707 | −16.981 | −25.836 | 77.976 | 1.00 | 223.61 | N |
| ATOM | 5231 | CA | TRP | B | 1707 | −18.401 | −25.676 | 77.801 | 1.00 | 224.36 | C |
| ATOM | 5232 | C | TRP | B | 1707 | −18.796 | −24.257 | 77.529 | 1.00 | 224.98 | C |
| ATOM | 5233 | O | TRP | B | 1707 | −18.728 | −23.399 | 78.405 | 1.00 | 224.93 | O |
| ATOM | 5234 | CB | TRP | B | 1707 | −19.139 | −26.201 | 79.018 | 1.00 | 224.30 | C |
| ATOM | 5235 | CG | TRP | B | 1707 | −20.456 | −26.759 | 78.680 | 1.00 | 224.26 | C |
| ATOM | 5236 | CD1 | TRP | B | 1707 | −20.774 | −28.075 | 78.552 | 1.00 | 224.14 | C |
| ATOM | 5237 | CD2 | TRP | B | 1707 | −21.650 | −26.023 | 78.408 | 1.00 | 224.20 | C |
| ATOM | 5238 | NE1 | TRP | B | 1707 | −22.102 | −28.210 | 78.232 | 1.00 | 224.09 | N |
| ATOM | 5239 | CE2 | TRP | B | 1707 | −22.664 | −26.965 | 78.134 | 1.00 | 224.19 | C |
| ATOM | 5240 | CE3 | TRP | B | 1707 | −21.966 | −24.657 | 78.380 | 1.00 | 224.02 | C |
| ATOM | 5241 | CZ2 | TRP | B | 1707 | −23.975 | −26.588 | 77.827 | 1.00 | 224.23 | C |
| ATOM | 5242 | CZ3 | TRP | B | 1707 | −23.264 | −24.280 | 78.071 | 1.00 | 224.11 | C |
| ATOM | 5243 | CH2 | TRP | B | 1707 | −24.254 | −25.244 | 77.798 | 1.00 | 224.37 | C |
| ATOM | 5244 | N | ASP | B | 1708 | −19.221 | −24.035 | 76.294 | 1.00 | 225.98 | N |
| ATOM | 5245 | CA | ASP | B | 1708 | −19.655 | −22.730 | 75.838 | 1.00 | 226.97 | C |
| ATOM | 5246 | C | ASP | B | 1708 | −21.180 | −22.696 | 75.806 | 1.00 | 227.55 | C |
| ATOM | 5247 | O | ASP | B | 1708 | −21.834 | −23.735 | 75.669 | 1.00 | 227.46 | O |
| ATOM | 5248 | CB | ASP | B | 1708 | −19.091 | −22.430 | 74.436 | 1.00 | 227.06 | C |
| ATOM | 5249 | CG | ASP | B | 1708 | −17.569 | −22.589 | 74.350 | 1.00 | 227.32 | C |
| ATOM | 5250 | CD1 | ASP | B | 1708 | −17.105 | −23.458 | 73.575 | 1.00 | 227.58 | O |
| ATOM | 5251 | CD2 | ASP | B | 1708 | −16.837 | −21.843 | 75.042 | 1.00 | 227.38 | O |
| ATOM | 5252 | N | TYR | B | 1709 | −21.736 | −21.497 | 75.938 | 1.00 | 228.43 | N |
| ATOM | 5253 | CA | TYR | B | 1709 | −23.167 | −21.291 | 75.760 | 1.00 | 229.27 | C |
| ATOM | 5254 | C | TYR | B | 1709 | −23.452 | −20.289 | 74.624 | 1.00 | 229.55 | C |
| ATOM | 5255 | O | TYR | B | 1709 | −24.082 | −20.660 | 73.625 | 1.00 | 229.65 | O |
| ATOM | 5256 | CB | TYR | B | 1709 | −23.822 | −20.875 | 77.082 | 1.00 | 229.59 | C |
| ATOM | 5257 | CG | TYR | B | 1709 | −25.312 | −20.585 | 77.005 | 1.00 | 230.27 | C |
| ATOM | 5258 | CD1 | TYR | B | 1709 | −26.261 | −21.583 | 77.266 | 1.00 | 230.09 | C |
| ATOM | 5259 | CD2 | TYR | B | 1709 | −25.775 | −19.296 | 76.696 | 1.00 | 231.17 | C |
| ATOM | 5260 | CE1 | TYR | B | 1709 | −27.641 | −21.304 | 77.205 | 1.00 | 230.35 | C |
| ATOM | 5261 | CE2 | TYR | B | 1709 | −27.147 | −19.008 | 76.630 | 1.00 | 231.25 | C |
| ATOM | 5262 | CZ | TYR | B | 1709 | −28.072 | −20.012 | 76.884 | 1.00 | 230.73 | C |
| ATOM | 5263 | OH | TYR | B | 1709 | −29.415 | −19.705 | 76.817 | 1.00 | 230.55 | O |
| ATOM | 5264 | N | GLY | B | 1710 | −22.981 | −19.046 | 74.775 | 1.00 | 229.71 | N |
| ATOM | 5265 | CA | GLY | B | 1710 | −23.213 | −17.967 | 73.801 | 1.00 | 230.00 | C |
| ATOM | 5266 | C | GLY | B | 1710 | −23.953 | −18.326 | 72.513 | 1.00 | 230.31 | C |
| ATOM | 5267 | O | GLY | B | 1710 | −23.347 | −18.842 | 71.567 | 1.00 | 230.38 | O |
| ATOM | 5268 | N | MET | B | 1711 | −25.265 | −18.065 | 72.490 | 1.00 | 230.53 | N |
| ATOM | 5269 | CA | MET | B | 1711 | −26.115 | −18.227 | 71.291 | 1.00 | 230.68 | C |
| ATOM | 5270 | C | MET | B | 1711 | −26.983 | −16.968 | 71.082 | 1.00 | 230.74 | C |
| ATOM | 5271 | O | MET | B | 1711 | −26.754 | −16.185 | 70.147 | 1.00 | 230.77 | O |
| ATOM | 5272 | CB | MET | B | 1711 | −26.993 | −19.488 | 71.403 | 1.00 | 230.66 | C |
| ATOM | 5273 | CG | MET | B | 1711 | −27.409 | −20.133 | 70.062 | 1.00 | 230.70 | C |
| ATOM | 5274 | SD | MET | B | 1711 | −29.037 | −19.720 | 69.356 | 1.00 | 230.60 | S |
| ATOM | 5275 | CE | MET | B | 1711 | −30.197 | −20.390 | 70.554 | 1.00 | 230.21 | C |
| ATOM | 5276 | N | SER | B | 1712 | −27.972 | −16.791 | 71.963 | 1.00 | 230.69 | N |
| ATOM | 5277 | CA | SER | B | 1712 | −28.812 | −15.585 | 72.022 | 1.00 | 230.55 | C |
| ATOM | 5278 | C | SER | B | 1712 | −29.528 | −15.494 | 73.381 | 1.00 | 230.43 | C |
| ATOM | 5279 | O | SER | B | 1712 | −29.691 | −14.401 | 73.948 | 1.00 | 230.41 | O |
| ATOM | 5280 | CB | SER | B | 1712 | −29.828 | −15.555 | 70.867 | 1.00 | 230.54 | C |
| ATOM | 5281 | OG | SER | B | 1712 | −30.600 | −16.744 | 70.821 | 1.00 | 230.40 | O |
| ATOM | 5282 | N | SER | B | 1713 | −29.922 | −16.661 | 73.894 | 1.00 | 230.12 | N |
| ATOM | 5283 | CA | SER | B | 1713 | −30.700 | −16.794 | 75.127 | 1.00 | 229.76 | C |
| ATOM | 5284 | C | SER | B | 1713 | −29.838 | −16.737 | 76.392 | 1.00 | 229.75 | C |
| ATOM | 5285 | O | SER | B | 1713 | −30.334 | −16.897 | 77.513 | 1.00 | 229.60 | O |
| ATOM | 5286 | CB | SER | B | 1713 | −31.483 | −18.101 | 75.074 | 1.00 | 229.58 | C |
| ATOM | 5287 | OG | SER | B | 1713 | −30.735 | −19.083 | 74.381 | 1.00 | 228.91 | O |
| ATOM | 5288 | N | GLY | B | 1725 | −22.161 | −9.815 | 79.352 | 1.00 | 193.00 | N |
| ATOM | 5289 | CA | GLY | B | 1725 | −22.448 | −11.272 | 79.286 | 1.00 | 193.22 | C |
| ATOM | 5290 | C | GLY | B | 1725 | −21.739 | −12.069 | 80.373 | 1.00 | 193.23 | C |
| ATOM | 5291 | O | GLY | B | 1725 | −20.508 | −12.179 | 80.367 | 1.00 | 193.36 | O |
| ATOM | 5292 | N | SER | B | 1726 | −22.522 | −12.649 | 81.286 | 1.00 | 193.14 | N |
| ATOM | 5293 | CA | SER | B | 1726 | −21.997 | −13.260 | 82.522 | 1.00 | 192.82 | C |
| ATOM | 5294 | C | SER | B | 1726 | −21.571 | −14.738 | 82.418 | 1.00 | 192.60 | C |
| ATOM | 5295 | O | SER | B | 1726 | −21.107 | −15.307 | 83.408 | 1.00 | 192.49 | O |
| ATOM | 5296 | CB | SER | B | 1726 | −22.987 | −13.056 | 83.692 | 1.00 | 192.86 | C |
| ATOM | 5297 | OG | SER | B | 1726 | −22.986 | −11.711 | 84.172 | 1.00 | 192.44 | O |
| ATOM | 5298 | N | VAL | B | 1727 | −21.711 | −15.337 | 81.229 | 1.00 | 192.39 | N |
| ATOM | 5299 | CA | VAL | B | 1727 | −21.405 | −16.772 | 80.973 | 1.00 | 192.21 | C |
| ATOM | 5300 | C | VAL | B | 1727 | −20.201 | −17.357 | 81.755 | 1.00 | 192.12 | C |
| ATOM | 5301 | O | VAL | B | 1727 | −19.165 | −16.684 | 81.868 | 1.00 | 192.28 | O |
| ATOM | 5302 | CB | VAL | B | 1727 | −21.211 | −17.063 | 79.448 | 1.00 | 192.17 | C |
| ATOM | 5303 | CG1 | VAL | B | 1727 | −22.546 | −17.374 | 78.772 | 1.00 | 191.88 | C |
| ATOM | 5304 | CG2 | VAL | B | 1727 | −20.473 | −15.911 | 78.749 | 1.00 | 192.11 | C |
| ATOM | 5305 | N | PRO | B | 1728 | −20.339 | −18.599 | 82.308 | 1.00 | 191.80 | N |
| ATOM | 5306 | CA | PRO | B | 1728 | −19.239 | −19.221 | 83.059 | 1.00 | 191.48 | C |
| ATOM | 5307 | C | PRO | B | 1728 | −18.291 | −20.086 | 82.224 | 1.00 | 191.21 | C |
| ATOM | 5308 | O | PRO | B | 1728 | −18.700 | −20.727 | 81.257 | 1.00 | 191.13 | O |
| ATOM | 5309 | CB | PRO | B | 1728 | −19.967 | −20.094 | 84.092 | 1.00 | 191.38 | C |
| ATOM | 5310 | CG | PRO | B | 1728 | −21.274 | −20.440 | 83.458 | 1.00 | 191.40 | C |
| ATOM | 5311 | CD | PRO | B | 1728 | −21.528 | −19.474 | 82.303 | 1.00 | 191.71 | C |
| ATOM | 5312 | N | GLN | B | 1729 | −17.021 | −20.070 | 82.612 | 1.00 | 191.00 | N |
| ATOM | 5313 | CA | GLN | B | 1729 | −16.022 | −21.012 | 82.121 | 1.00 | 190.65 | C |
| ATOM | 5314 | C | GLN | B | 1729 | −16.276 | −22.335 | 82.837 | 1.00 | 190.31 | C |
| ATOM | 5315 | O | GLN | B | 1729 | −16.724 | −22.349 | 83.988 | 1.00 | 190.36 | O |
| ATOM | 5316 | CB | GLN | B | 1729 | −14.612 | −20.482 | 82.447 | 1.00 | 190.82 | C |
| ATOM | 5317 | CG | GLN | B | 1729 | −13.439 | −21.474 | 82.302 | 1.00 | 190.66 | C |
| ATOM | 5318 | CD | GLN | B | 1729 | −12.300 | −21.224 | 83.302 | 1.00 | 190.55 | C |
| ATOM | 5319 | OE1 | GLN | B | 1729 | −11.568 | −22.148 | 83.657 | 1.00 | 190.02 | O |
| ATOM | 5320 | NE2 | GLN | B | 1729 | −12.154 | −19.977 | 83.759 | 1.00 | 190.31 | N |
| ATOM | 5321 | N | PHE | B | 1730 | −16.015 | −23.437 | 82.145 | 1.00 | 189.72 | N |
| ATOM | 5322 | CA | PHE | B | 1730 | −15.983 | −24.752 | 82.767 | 1.00 | 189.11 | C |
| ATOM | 5323 | C | PHE | B | 1730 | −14.736 | −25.473 | 82.249 | 1.00 | 188.93 | C |
| ATOM | 5324 | O | PHE | B | 1730 | −14.267 | −25.172 | 81.144 | 1.00 | 189.09 | O |
| ATOM | 5325 | CB | PHE | B | 1730 | −17.245 | −25.528 | 82.413 | 1.00 | 188.91 | C |
| ATOM | 5326 | CG | PHE | B | 1730 | −18.379 | −25.362 | 83.390 | 1.00 | 188.37 | C |
| ATOM | 5327 | CD1 | PHE | B | 1730 | −18.690 | −26.383 | 84.284 | 1.00 | 187.80 | C |
| ATOM | 5328 | CD2 | PHE | B | 1730 | −19.166 | −24.212 | 83.386 | 1.00 | 187.94 | C |
| ATOM | 5329 | CE1 | PHE | B | 1730 | −19.750 | −26.250 | 85.172 | 1.00 | 187.62 | C |
| ATOM | 5330 | CE2 | PHE | B | 1730 | −20.230 | −24.069 | 84.277 | 1.00 | 187.70 | C |
| ATOM | 5331 | CZ | PHE | B | 1730 | −20.523 | −25.091 | 85.168 | 1.00 | 187.79 | C |
| ATOM | 5332 | N | LYS | B | 1731 | −14.202 | −26.418 | 83.027 | 1.00 | 188.47 | N |
| ATOM | 5333 | CA | LYS | B | 1731 | −12.930 | −27.063 | 82.679 | 1.00 | 188.06 | C |
| ATOM | 5334 | C | LYS | B | 1731 | −12.923 | −28.594 | 82.816 | 1.00 | 188.06 | C |
| ATOM | 5335 | O | LYS | B | 1731 | −11.978 | −29.167 | 83.364 | 1.00 | 187.98 | O |
| ATOM | 5336 | CB | LYS | B | 1731 | −11.790 | −26.445 | 83.495 | 1.00 | 187.86 | C |
| ATOM | 5337 | CG | LYS | B | 1731 | −10.436 | −26.510 | 82.816 | 1.00 | 187.11 | C |
| ATOM | 5338 | CD | LYS | B | 1731 | −9.307 | −26.418 | 83.826 | 1.00 | 186.15 | C |
| ATOM | 5339 | CE | LYS | B | 1731 | −9.182 | −27.693 | 84.638 | 1.00 | 185.61 | C |
| ATOM | 5340 | NZ | LYS | B | 1731 | −9.043 | −28.876 | 83.753 | 1.00 | 184.87 | N |
| ATOM | 5341 | N | LYS | B | 1732 | −13.972 | −29.242 | 82.299 | 1.00 | 188.11 | N |
| ATOM | 5342 | CA | LYS | B | 1732 | −14.093 | −30.713 | 82.265 | 1.00 | 188.11 | C |
| ATOM | 5343 | C | LYS | B | 1732 | −13.149 | −31.352 | 81.238 | 1.00 | 188.59 | C |
| ATOM | 5344 | O | LYS | B | 1732 | −12.535 | −30.647 | 80.434 | 1.00 | 188.47 | O |
| ATOM | 5345 | CB | LYS | B | 1732 | −15.538 | −31.139 | 81.974 | 1.00 | 187.70 | C |
| ATOM | 5346 | CG | LYS | B | 1732 | −16.441 | −31.238 | 83.185 | 1.00 | 186.42 | C |
| ATOM | 5347 | CD | LYS | B | 1732 | −17.879 | −31.447 | 82.765 | 1.00 | 184.01 | C |
| ATOM | 5348 | CE | LYS | B | 1732 | −18.668 | −30.159 | 82.817 | 1.00 | 182.50 | C |
| ATOM | 5349 | NZ | LYS | B | 1732 | −19.856 | −30.243 | 81.954 | 1.00 | 180.84 | N |
| ATOM | 5350 | N | VAL | B | 1733 | −13.047 | −32.685 | 81.275 | 1.00 | 189.24 | N |
| ATOM | 5351 | CA | VAL | B | 1733 | −12.142 | −33.454 | 80.400 | 1.00 | 189.80 | C |
| ATOM | 5352 | C | VAL | B | 1733 | −12.897 | −34.032 | 79.206 | 1.00 | 190.32 | C |
| ATOM | 5353 | O | VAL | B | 1733 | −14.083 | −34.319 | 79.319 | 1.00 | 190.42 | O |
| ATOM | 5354 | CB | VAL | B | 1733 | −11.487 | −34.653 | 81.135 | 1.00 | 189.67 | C |
| ATOM | 5355 | CG1 | VAL | B | 1733 | −10.027 | −34.768 | 80.740 | 1.00 | 189.73 | C |
| ATOM | 5356 | CG2 | VAL | B | 1733 | −11.623 | −34.533 | 82.644 | 1.00 | 189.41 | C |
| ATOM | 5357 | N | VAL | B | 1734 | −12.212 | −34.191 | 78.070 | 1.00 | 190.91 | N |
| ATOM | 5358 | CA | VAL | B | 1734 | −12.785 | −34.849 | 76.884 | 1.00 | 191.46 | C |
| ATOM | 5359 | C | VAL | B | 1734 | −11.775 | −35.768 | 76.214 | 1.00 | 192.01 | C |
| ATOM | 5360 | O | VAL | B | 1734 | −10.565 | −35.589 | 76.382 | 1.00 | 192.08 | O |
| ATOM | 5361 | CB | VAL | B | 1734 | −13.292 | −33.846 | 75.802 | 1.00 | 191.38 | C |
| ATOM | 5362 | CG1 | VAL | B | 1734 | −14.687 | −33.329 | 76.129 | 1.00 | 191.04 | C |
| ATOM | 5363 | CG2 | VAL | B | 1734 | −12.288 | −32.711 | 75.573 | 1.00 | 191.38 | C |
| ATOM | 5364 | N | PHE | B | 1735 | −12.284 | −36.744 | 75.462 | 1.00 | 192.58 | N |
| ATOM | 5365 | CA | PHE | B | 1735 | −11.475 | −37.499 | 74.510 | 1.00 | 193.25 | C |
| ATOM | 5366 | C | PHE | B | 1735 | −11.199 | −36.658 | 73.263 | 1.00 | 193.80 | C |
| ATOM | 5367 | O | PHE | B | 1735 | −11.989 | −35.773 | 72.927 | 1.00 | 193.95 | O |
| ATOM | 5368 | CB | PHE | B | 1735 | −12.213 | −38.753 | 74.088 | 1.00 | 193.19 | C |
| ATOM | 5369 | CG | PHE | B | 1735 | −12.101 | −39.878 | 75.056 | 1.00 | 193.45 | C |
| ATOM | 5370 | CD1 | PHE | B | 1735 | −10.896 | −40.551 | 75.222 | 1.00 | 193.69 | C |
| ATOM | 5371 | CD2 | PHE | B | 1735 | −13.209 | −40.297 | 75.780 | 1.00 | 193.73 | C |
| ATOM | 5372 | CE1 | PHE | B | 1735 | −10.790 | −41.622 | 76.116 | 1.00 | 193.79 | C |
| ATOM | 5373 | CE2 | PHE | B | 1735 | −13.114 | −41.372 | 76.669 | 1.00 | 193.94 | C |
| ATOM | 5374 | CZ | PHE | B | 1735 | −11.902 | −42.032 | 76.841 | 1.00 | 193.66 | C |
| ATOM | 5375 | N | GLN | B | 1736 | −10.091 | −36.931 | 72.573 | 1.00 | 194.41 | N |
| ATOM | 5376 | CA | GLN | B | 1736 | −9.748 | −36.197 | 71.351 | 1.00 | 195.12 | C |
| ATOM | 5377 | C | GLN | B | 1736 | −8.930 | −37.069 | 70.429 | 1.00 | 195.59 | C |
| ATOM | 5378 | O | GLN | B | 1736 | −7.974 | −37.705 | 70.863 | 1.00 | 195.52 | O |
| ATOM | 5379 | CB | GLN | B | 1736 | −8.963 | −34.923 | 71.679 | 1.00 | 195.20 | C |
| ATOM | 5380 | CG | GLN | B | 1736 | −8.612 | −34.043 | 70.469 | 1.00 | 195.86 | C |
| ATOM | 5381 | CD | GLN | B | 1736 | −9.615 | −32.921 | 70.218 | 1.00 | 196.66 | C |
| ATOM | 5382 | OE1 | GLN | B | 1736 | −9.945 | −32.604 | 69.067 | 1.00 | 196.53 | O |
| ATOM | 5383 | NE2 | GLN | B | 1736 | −10.095 | −32.306 | 71.296 | 1.00 | 197.06 | N |
| ATOM | 5384 | N | GLU | B | 1737 | −9.311 | −37.089 | 69.156 | 1.00 | 196.43 | N |
| ATOM | 5385 | CA | GLU | B | 1737 | −8.596 | −37.854 | 68.135 | 1.00 | 197.55 | C |
| ATOM | 5386 | C | GLU | B | 1737 | −7.395 | −37.072 | 67.609 | 1.00 | 197.79 | C |
| ATOM | 5387 | O | GLU | B | 1737 | −7.528 | −35.912 | 67.230 | 1.00 | 198.07 | O |
| ATOM | 5388 | CB | GLU | B | 1737 | −9.540 | −38.202 | 66.979 | 1.00 | 197.51 | C |
| ATOM | 5389 | CG | GLU | B | 1737 | −8.947 | −39.121 | 65.902 | 1.00 | 198.26 | C |
| ATOM | 5390 | CD | GLU | B | 1737 | −10.010 | −39.757 | 64.997 | 1.00 | 198.58 | C |
| ATOM | 5391 | OE1 | GLU | B | 1737 | −9.634 | −40.338 | 63.950 | 1.00 | 199.65 | O |
| ATOM | 5392 | OE2 | GLU | B | 1737 | −11.219 | −39.685 | 65.331 | 1.00 | 200.06 | O |
| ATOM | 5393 | N | PHE | B | 1738 | −6.225 | −37.705 | 67.589 | 1.00 | 198.19 | N |
| ATOM | 5394 | CA | PHE | B | 1738 | −5.019 | −37.074 | 67.036 | 1.00 | 198.49 | C |
| ATOM | 5395 | C | PHE | B | 1738 | −4.626 | −37.688 | 65.685 | 1.00 | 198.41 | C |
| ATOM | 5396 | O | PHE | B | 1738 | −5.392 | −38.472 | 65.118 | 1.00 | 198.36 | O |
| ATOM | 5397 | CB | PHE | B | 1738 | −3.860 | −37.098 | 68.047 | 1.00 | 198.76 | C |
| ATOM | 5398 | CG | PHE | B | 1738 | −3.808 | −35.884 | 68.944 | 1.00 | 199.20 | C |
| ATOM | 5399 | CD1 | PHE | B | 1738 | −2.927 | −34.838 | 68.669 | 1.00 | 199.52 | C |
| ATOM | 5400 | CD2 | PHE | B | 1738 | −4.643 | −35.784 | 70.059 | 1.00 | 199.72 | C |
| ATOM | 5401 | CE1 | PHE | B | 1738 | −2.871 | −33.711 | 69.494 | 1.00 | 199.84 | C |
| ATOM | 5402 | CE2 | PHE | B | 1738 | −4.599 | −34.658 | 70.892 | 1.00 | 200.11 | C |
| ATOM | 5403 | CZ | PHE | B | 1738 | −3.709 | −33.619 | 70.608 | 1.00 | 199.95 | C |
| ATOM | 5404 | N | THR | B | 1739 | −3.443 | −37.330 | 65.181 | 1.00 | 198.26 | N |
| ATOM | 5405 | CA | THR | B | 1739 | −3.050 | −37.676 | 63.816 | 1.00 | 198.06 | C |
| ATOM | 5406 | C | THR | B | 1739 | −2.087 | −38.863 | 63.702 | 1.00 | 198.00 | C |
| ATOM | 5407 | O | THR | B | 1739 | −2.138 | −39.605 | 62.725 | 1.00 | 197.91 | O |
| ATOM | 5408 | CB | THR | B | 1739 | −2.476 | −36.455 | 63.078 | 1.00 | 198.01 | C |
| ATOM | 5409 | OG1 | THR | B | 1739 | −2.701 | −36.591 | 61.672 | 1.00 | 198.15 | O |
| ATOM | 5410 | CG2 | THR | B | 1739 | −0.991 | −36.301 | 63.342 | 1.00 | 197.84 | C |
| ATOM | 5411 | N | ASP | B | 1740 | −1.206 | −39.022 | 64.685 | 1.00 | 198.05 | N |
| ATOM | 5412 | CA | ASP | B | 1740 | −0.259 | −40.144 | 64.717 | 1.00 | 198.24 | C |
| ATOM | 5413 | C | ASP | B | 1740 | 0.318 | −40.348 | 66.124 | 1.00 | 198.11 | C |
| ATOM | 5414 | O | ASP | B | 1740 | −0.251 | −39.855 | 67.103 | 1.00 | 198.21 | O |
| ATOM | 5415 | CB | ASP | B | 1740 | 0.853 | −39.997 | 63.649 | 1.00 | 198.44 | C |
| ATOM | 5416 | CG | ASP | B | 1740 | 1.503 | −38.602 | 63.626 | 1.00 | 199.14 | C |
| ATOM | 5417 | OD1 | ASP | B | 1740 | 1.382 | −37.846 | 64.618 | 1.00 | 200.07 | O |
| ATOM | 5418 | OD2 | ASP | B | 1740 | 2.148 | −38.263 | 62.603 | 1.00 | 199.46 | O |
| ATOM | 5419 | N | GLY | B | 1741 | 1.426 | −41.088 | 66.222 | 1.00 | 197.91 | N |
| ATOM | 5420 | CA | GLY | B | 1741 | 2.111 | −41.326 | 67.501 | 1.00 | 197.55 | C |
| ATOM | 5421 | C | GLY | B | 1741 | 2.730 | −40.082 | 68.126 | 1.00 | 197.28 | C |
| ATOM | 5422 | O | GLY | B | 1741 | 2.815 | −39.973 | 69.351 | 1.00 | 197.21 | O |
| ATOM | 5423 | N | SER | B | 1742 | 3.153 | −39.144 | 67.278 | 1.00 | 197.00 | N |
| ATOM | 5424 | CA | SER | B | 1742 | 3.731 | −37.865 | 67.707 | 1.00 | 196.67 | C |
| ATOM | 5425 | C | SER | B | 1742 | 2.678 | −36.890 | 68.257 | 1.00 | 196.40 | C |
| ATOM | 5426 | O | SER | B | 1742 | 2.917 | −35.677 | 68.328 | 1.00 | 196.26 | O |
| ATOM | 5427 | CB | SER | B | 1742 | 4.502 | −37.225 | 66.542 | 1.00 | 196.71 | C |
| ATOM | 5428 | OG | SER | B | 1742 | 5.099 | −35.995 | 66.920 | 1.00 | 196.79 | O |
| ATOM | 5429 | N | PHE | B | 1743 | 1.523 | −37.437 | 68.644 | 1.00 | 196.11 | N |
| ATOM | 5430 | CA | PHE | B | 1743 | 0.402 | −36.678 | 69.202 | 1.00 | 195.88 | C |
| ATOM | 5431 | C | PHE | B | 1743 | 0.521 | −35.167 | 68.976 | 1.00 | 195.85 | C |
| ATOM | 5432 | O | PHE | B | 1743 | 0.677 | −34.396 | 69.924 | 1.00 | 195.89 | O |
| ATOM | 5433 | CB | PHE | B | 1743 | 0.235 | −36.989 | 70.695 | 1.00 | 195.74 | C |
| ATOM | 5434 | CG | PHE | B | 1743 | −0.383 | −38.333 | 70.981 | 1.00 | 195.51 | C |
| ATOM | 5435 | CD1 | PHE | B | 1743 | −1.764 | −38.465 | 71.120 | 1.00 | 195.29 | C |
| ATOM | 5436 | CD2 | PHE | B | 1743 | 0.412 | −39.463 | 71.134 | 1.00 | 195.18 | C |
| ATOM | 5437 | CE1 | PHE | B | 1743 | −2.342 | −39.705 | 71.396 | 1.00 | 194.90 | C |
| ATOM | 5438 | CE2 | PHE | B | 1743 | −0.156 | −40.705 | 71.409 | 1.00 | 195.04 | C |
| ATOM | 5439 | CZ | PHE | B | 1743 | −1.536 | −40.825 | 71.541 | 1.00 | 195.04 | C |
| ATOM | 5440 | N | THR | B | 1744 | 0.461 | −34.755 | 67.712 | 1.00 | 195.77 | N |
| ATOM | 5441 | CA | THR | B | 1744 | 0.520 | −33.336 | 67.372 | 1.00 | 195.58 | C |
| ATOM | 5442 | C | THR | B | 1744 | −0.755 | −32.804 | 66.699 | 1.00 | 195.86 | C |
| ATOM | 5443 | O | THR | B | 1744 | −1.476 | −32.029 | 67.326 | 1.00 | 195.98 | O |
| ATOM | 5444 | CB | THR | B | 1744 | 1.792 | −32.968 | 66.591 | 1.00 | 195.34 | C |
| ATOM | 5445 | OG1 | THR | B | 1744 | 2.295 | −34.132 | 65.933 | 1.00 | 194.85 | O |
| ATOM | 5446 | CG2 | THR | B | 1744 | 2.847 | −32.466 | 67.546 | 1.00 | 194.98 | C |
| ATOM | 5447 | N | GLN | B | 1745 | −1.052 | −33.222 | 65.462 | 1.00 | 196.00 | N |
| ATOM | 5448 | CA | GLN | B | 1745 | −2.208 | −32.665 | 64.719 | 1.00 | 196.11 | C |
| ATOM | 5449 | C | GLN | B | 1745 | −3.523 | −32.900 | 65.450 | 1.00 | 195.81 | C |
| ATOM | 5450 | O | GLN | B | 1745 | −3.877 | −34.042 | 65.733 | 1.00 | 195.54 | O |
| ATOM | 5451 | CB | GLN | B | 1745 | −2.309 | −33.190 | 63.277 | 1.00 | 196.28 | C |
| ATOM | 5452 | CG | GLN | B | 1745 | −0.999 | −33.171 | 62.461 | 1.00 | 197.61 | C |
| ATOM | 5453 | CD | GLN | B | 1745 | −0.695 | −31.841 | 61.772 | 1.00 | 199.22 | C |
| ATOM | 5454 | OE1 | GLN | B | 1745 | −1.260 | −30.794 | 62.110 | 1.00 | 200.22 | O |
| ATOM | 5455 | NE2 | GLN | B | 1745 | 0.217 | −31.881 | 60.798 | 1.00 | 199.45 | N |
| ATOM | 5456 | N | PRO | B | 1746 | −4.263 | −31.813 | 65.735 | 1.00 | 195.82 | N |
| ATOM | 5457 | CA | PRO | B | 1746 | −5.422 | −31.953 | 66.599 | 1.00 | 195.84 | C |
| ATOM | 5458 | C | PRO | B | 1746 | −6.512 | −32.704 | 65.862 | 1.00 | 195.90 | C |
| ATOM | 5459 | O | PRO | B | 1746 | −7.468 | −33.150 | 66.489 | 1.00 | 196.00 | O |
| ATOM | 5460 | CB | PRO | B | 1746 | −5.844 | −30.503 | 66.865 | 1.00 | 195.85 | C |
| ATOM | 5461 | CG | PRO | B | 1746 | −5.376 | −29.748 | 65.670 | 1.00 | 195.86 | C |
| ATOM | 5462 | CD | PRO | B | 1746 | −4.103 | −30.430 | 65.240 | 1.00 | 195.91 | C |
| ATOM | 5463 | N | LEU | B | 1747 | −6.350 | −32.830 | 64.540 | 1.00 | 195.92 | N |
| ATOM | 5464 | CA | LEU | B | 1747 | −7.260 | −33.578 | 63.661 | 1.00 | 195.84 | C |
| ATOM | 5465 | C | LEU | B | 1747 | −8.637 | −32.895 | 63.515 | 1.00 | 195.91 | C |
| ATOM | 5466 | O | LEU | B | 1747 | −9.670 | −33.540 | 63.703 | 1.00 | 195.92 | O |
| ATOM | 5467 | CB | LEU | B | 1747 | −7.390 | −35.036 | 64.156 | 1.00 | 195.68 | C |
| ATOM | 5468 | CG | LEU | B | 1747 | −7.487 | −36.279 | 63.257 | 1.00 | 195.09 | C |
| ATOM | 5469 | CD1 | LEU | B | 1747 | −8.913 | −36.545 | 62.768 | 1.00 | 194.15 | C |
| ATOM | 5470 | CD2 | LEU | B | 1747 | −6.477 | −36.249 | 62.098 | 1.00 | 194.65 | C |
| ATOM | 5471 | N | TYR | B | 1748 | −8.633 | −31.598 | 63.180 | 1.00 | 195.97 | N |
| ATOM | 5472 | CA | TYR | B | 1748 | −9.854 | −30.777 | 63.017 | 1.00 | 196.07 | C |
| ATOM | 5473 | C | TYR | B | 1748 | −11.134 | −31.603 | 62.814 | 1.00 | 195.71 | C |
| ATOM | 5474 | O | TYR | B | 1748 | −11.222 | −32.408 | 61.879 | 1.00 | 195.69 | O |
| ATOM | 5475 | CB | TYR | B | 1748 | −9.705 | −29.823 | 61.822 | 1.00 | 196.64 | C |
| ATOM | 5476 | CG | TYR | B | 1748 | −9.175 | −28.415 | 62.090 | 1.00 | 197.57 | C |
| ATOM | 5477 | CD1 | TYR | B | 1748 | −7.804 | −28.124 | 61.980 | 1.00 | 198.23 | C |
| ATOM | 5478 | CD2 | TYR | B | 1748 | −10.053 | −27.357 | 62.382 | 1.00 | 198.42 | C |
| ATOM | 5479 | CE1 | TYR | B | 1748 | −7.312 | −26.818 | 62.192 | 1.00 | 198.23 | C |
| ATOM | 5480 | CE2 | TYR | B | 1748 | −9.572 | −26.045 | 62.598 | 1.00 | 198.60 | C |
| ATOM | 5481 | CZ | TYR | B | 1748 | −8.200 | −25.785 | 62.501 | 1.00 | 198.09 | C |
| ATOM | 5482 | OH | TYR | B | 1748 | −7.720 | −24.504 | 62.711 | 1.00 | 197.38 | O |
| ATOM | 5483 | N | ARG | B | 1749 | −12.122 | −31.391 | 63.683 | 1.00 | 195.24 | N |
| ATOM | 5484 | CA | ARG | B | 1749 | −13.412 | −32.079 | 63.598 | 1.00 | 194.66 | C |
| ATOM | 5485 | C | ARG | B | 1749 | −14.219 | −31.624 | 62.387 | 1.00 | 194.62 | C |
| ATOM | 5486 | O | ARG | B | 1749 | −14.679 | −30.480 | 62.329 | 1.00 | 194.69 | O |
| ATOM | 5487 | CB | ARG | B | 1749 | −14.207 | −31.856 | 64.880 | 1.00 | 194.41 | C |
| ATOM | 5488 | CG | ARG | B | 1749 | −13.740 | −32.713 | 66.017 | 1.00 | 193.81 | C |
| ATOM | 5489 | CD | ARG | B | 1749 | −14.009 | −34.174 | 65.695 | 1.00 | 192.94 | C |
| ATOM | 5490 | NE | ARG | B | 1749 | −14.416 | −34.943 | 66.870 | 1.00 | 192.19 | N |
| ATOM | 5491 | CZ | ARG | B | 1749 | −15.580 | −34.799 | 67.507 | 1.00 | 191.38 | C |
| ATOM | 5492 | NH1 | ARG | B | 1749 | −16.477 | −33.895 | 67.107 | 1.00 | 190.59 | N |
| ATOM | 5493 | NH2 | ARG | B | 1749 | −15.840 | −35.557 | 68.566 | 1.00 | 190.68 | N |
| ATOM | 5494 | N | GLY | B | 1750 | −14.389 | −32.525 | 61.423 | 1.00 | 194.44 | N |
| ATOM | 5495 | CA | GLY | B | 1750 | −14.995 | −32.169 | 60.141 | 1.00 | 194.29 | C |
| ATOM | 5496 | C | GLY | B | 1750 | −16.484 | −31.901 | 60.202 | 1.00 | 194.19 | C |
| ATOM | 5497 | O | GLY | B | 1750 | −17.186 | −32.505 | 61.007 | 1.00 | 194.11 | O |
| ATOM | 5498 | N | GLU | B | 1751 | −16.953 | −31.003 | 59.335 | 1.00 | 194.17 | N |
| ATOM | 5499 | CA | GLU | B | 1751 | −18.379 | −30.649 | 59.188 | 1.00 | 194.40 | C |
| ATOM | 5500 | C | GLU | B | 1751 | −19.331 | −31.827 | 59.225 | 1.00 | 194.30 | C |
| ATOM | 5501 | O | GLU | B | 1751 | −20.517 | −31.672 | 59.540 | 1.00 | 194.19 | O |
| ATOM | 5502 | CB | GLU | B | 1751 | −18.616 | −29.943 | 57.855 | 1.00 | 194.47 | C |
| ATOM | 5503 | CG | GLU | B | 1751 | −18.234 | −28.487 | 57.827 | 1.00 | 195.94 | C |
| ATOM | 5504 | CD | GLU | B | 1751 | −16.755 | −28.256 | 57.571 | 1.00 | 198.14 | C |
| ATOM | 5505 | OE1 | GLU | B | 1751 | −16.007 | −29.255 | 57.441 | 1.00 | 199.20 | O |
| ATOM | 5506 | OE2 | GLU | B | 1751 | −16.345 | −27.069 | 57.496 | 1.00 | 198.90 | O |
| ATOM | 5507 | N | LEU | B | 1752 | −18.806 | −32.991 | 58.853 | 1.00 | 194.39 | N |
| ATOM | 5508 | CA | LEU | B | 1752 | −19.587 | −34.215 | 58.786 | 1.00 | 194.56 | C |
| ATOM | 5509 | C | LEU | B | 1752 | −19.776 | −34.856 | 60.149 | 1.00 | 194.65 | C |
| ATOM | 5510 | O | LEU | B | 1752 | −20.754 | −35.580 | 60.344 | 1.00 | 195.01 | O |
| ATOM | 5511 | CB | LEU | B | 1752 | −19.009 | −35.227 | 57.779 | 1.00 | 194.48 | C |
| ATOM | 5512 | CG | LEU | B | 1752 | −17.514 | −35.472 | 57.500 | 1.00 | 194.94 | C |
| ATOM | 5513 | CD1 | LEU | B | 1752 | −16.938 | −34.403 | 56.572 | 1.00 | 195.63 | C |
| ATOM | 5514 | CD2 | LEU | B | 1752 | −16.642 | −35.634 | 58.753 | 1.00 | 195.19 | C |
| ATOM | 5515 | N | ASN | B | 1753 | −18.852 | −34.601 | 61.083 | 1.00 | 194.51 | N |
| ATOM | 5516 | CA | ASN | B | 1753 | −19.016 | −35.066 | 62.470 | 1.00 | 194.34 | C |
| ATOM | 5517 | C | ASN | B | 1753 | −18.638 | −34.058 | 63.546 | 1.00 | 194.07 | C |
| ATOM | 5518 | O | ASN | B | 1753 | −17.691 | −34.258 | 64.311 | 1.00 | 194.09 | O |
| ATOM | 5519 | CB | ASN | B | 1753 | −18.354 | −36.437 | 62.718 | 1.00 | 194.47 | C |
| ATOM | 5520 | CG | ASN | B | 1753 | −16.922 | −36.510 | 62.214 | 1.00 | 195.09 | C |
| ATOM | 5521 | OD1 | ASN | B | 1753 | −16.300 | −35.486 | 61.889 | 1.00 | 195.68 | O |
| ATOM | 5522 | ND2 | ASN | B | 1753 | −16.385 | −37.734 | 62.149 | 1.00 | 195.22 | N |
| ATOM | 5523 | N | GLU | B | 1754 | −19.398 | −32.968 | 63.596 | 1.00 | 193.78 | N |
| ATOM | 5524 | CA | GLU | B | 1754 | −19.199 | −31.940 | 64.609 | 1.00 | 193.50 | C |
| ATOM | 5525 | C | GLU | B | 1754 | −20.342 | −31.952 | 65.592 | 1.00 | 192.80 | C |
| ATOM | 5526 | O | GLU | B | 1754 | −20.267 | −31.328 | 66.645 | 1.00 | 192.85 | O |
| ATOM | 5527 | CB | GLU | B | 1754 | −19.049 | −30.563 | 63.977 | 1.00 | 193.82 | C |
| ATOM | 5528 | CG | GLU | B | 1754 | −20.330 | −29.931 | 63.472 | 1.00 | 195.26 | C |
| ATOM | 5529 | CD | GLU | B | 1754 | −20.128 | −28.463 | 63.111 | 1.00 | 197.97 | C |
| ATOM | 5530 | OE1 | GLU | B | 1754 | −19.081 | −28.130 | 62.494 | 1.00 | 198.74 | O |
| ATOM | 5531 | OE2 | GLU | B | 1754 | −21.012 | −27.638 | 63.452 | 1.00 | 199.38 | O |
| ATOM | 5532 | N | HIS | B | 1755 | −21.404 | −32.661 | 65.223 | 1.00 | 192.02 | N |
| ATOM | 5533 | CA | HIS | B | 1755 | −22.457 | −33.052 | 66.157 | 1.00 | 191.30 | C |
| ATOM | 5534 | C | HIS | B | 1755 | −21.866 | −33.753 | 67.387 | 1.00 | 190.60 | C |
| ATOM | 5535 | O | HIS | B | 1755 | −22.297 | −33.535 | 68.520 | 1.00 | 190.32 | O |
| ATOM | 5536 | CB | HIS | B | 1755 | −23.503 | −33.943 | 65.450 | 1.00 | 191.44 | C |
| ATOM | 5537 | CG | HIS | B | 1755 | −22.964 | −35.239 | 64.903 | 1.00 | 191.38 | C |
| ATOM | 5538 | ND1 | HIS | B | 1755 | −21.880 | −35.309 | 64.053 | 1.00 | 191.34 | N |
| ATOM | 5539 | CD2 | HIS | B | 1755 | −23.395 | −36.514 | 65.060 | 1.00 | 191.32 | C |
| ATOM | 5540 | CE1 | HIS | B | 1755 | −21.653 | −36.572 | 63.731 | 1.00 | 191.10 | C |
| ATOM | 5541 | NE2 | HIS | B | 1755 | −22.561 | −37.323 | 64.325 | 1.00 | 191.03 | N |
| ATOM | 5542 | N | LEU | B | 1756 | −20.859 | −34.578 | 67.129 | 1.00 | 189.88 | N |
| ATOM | 5543 | CA | LEU | B | 1756 | −20.117 | −35.297 | 68.139 | 1.00 | 189.20 | C |
| ATOM | 5544 | C | LEU | B | 1756 | −19.653 | −34.324 | 69.216 | 1.00 | 188.69 | C |
| ATOM | 5545 | O | LEU | B | 1756 | −19.806 | −34.598 | 70.397 | 1.00 | 188.38 | O |
| ATOM | 5546 | CB | LEU | B | 1756 | −18.931 | −35.998 | 67.468 | 1.00 | 189.42 | C |
| ATOM | 5547 | CG | LEU | B | 1756 | −18.598 | −37.482 | 67.688 | 1.00 | 189.38 | C |
| ATOM | 5548 | CD1 | LEU | B | 1756 | −19.828 | −38.366 | 67.614 | 1.00 | 189.44 | C |
| ATOM | 5549 | CD2 | LEU | B | 1756 | −17.554 | −37.946 | 66.687 | 1.00 | 189.17 | C |
| ATOM | 5550 | N | GLY | B | 1757 | −19.083 | −33.197 | 68.792 | 1.00 | 188.32 | N |
| ATOM | 5551 | CA | GLY | B | 1757 | −18.872 | −32.018 | 69.654 | 1.00 | 188.08 | C |
| ATOM | 5552 | C | GLY | B | 1757 | −18.112 | −32.103 | 70.982 | 1.00 | 187.76 | C |
| ATOM | 5553 | O | GLY | B | 1757 | −16.880 | −31.880 | 71.011 | 1.00 | 188.03 | O |
| ATOM | 5554 | N | LEU | B | 1758 | −18.853 | −32.378 | 72.075 | 1.00 | 186.99 | N |
| ATOM | 5555 | CA | LEU | B | 1758 | −18.325 | −32.421 | 73.480 | 1.00 | 185.76 | C |
| ATOM | 5556 | C | LEU | B | 1758 | −18.033 | −33.839 | 74.010 | 1.00 | 184.72 | C |
| ATOM | 5557 | O | LEU | B | 1758 | −17.810 | −34.024 | 75.209 | 1.00 | 184.46 | O |
| ATOM | 5558 | CB | LEU | B | 1758 | −19.231 | −31.630 | 74.478 | 1.00 | 185.77 | C |
| ATOM | 5559 | CG | LEU | B | 1758 | −18.880 | −30.328 | 75.259 | 1.00 | 185.16 | C |
| ATOM | 5560 | CD1 | LEU | B | 1758 | −18.031 | −30.630 | 76.468 | 1.00 | 184.73 | C |
| ATOM | 5561 | CD2 | LEU | B | 1758 | −18.262 | −29.179 | 74.444 | 1.00 | 184.12 | C |
| ATOM | 5562 | N | LEU | B | 1759 | −18.040 | −34.821 | 73.108 | 1.00 | 183.42 | N |
| ATOM | 5563 | CA | LEU | B | 1759 | −17.555 | −36.156 | 73.412 | 1.00 | 182.36 | C |
| ATOM | 5564 | C | LEU | B | 1759 | −16.523 | −36.607 | 72.381 | 1.00 | 182.44 | C |
| ATOM | 5565 | O | LEU | B | 1759 | −16.077 | −35.806 | 71.560 | 1.00 | 182.39 | O |
| ATOM | 5566 | CB | LEU | B | 1759 | −18.716 | −37.144 | 73.538 | 1.00 | 181.85 | C |
| ATOM | 5567 | CG | LEU | B | 1759 | −19.486 | −37.771 | 72.382 | 1.00 | 180.00 | C |
| ATOM | 5568 | CD1 | LEU | B | 1759 | −19.204 | −37.064 | 71.123 | 1.00 | 179.10 | C |
| ATOM | 5569 | CD2 | LEU | B | 1759 | −19.156 | −39.226 | 72.226 | 1.00 | 178.06 | C |
| ATOM | 5570 | N | GLY | B | 1760 | −16.162 | −37.890 | 72.426 | 1.00 | 182.46 | N |
| ATOM | 5571 | CA | GLY | B | 1760 | −15.036 | −38.439 | 71.661 | 1.00 | 182.29 | C |
| ATOM | 5572 | C | GLY | B | 1760 | −15.319 | −38.874 | 70.239 | 1.00 | 182.16 | C |
| ATOM | 5573 | O | GLY | B | 1760 | −16.473 | −38.938 | 69.832 | 1.00 | 182.01 | O |
| ATOM | 5574 | N | PRO | B | 1761 | −14.258 | −39.186 | 69.473 | 1.00 | 182.26 | N |
| ATOM | 5575 | CA | PRO | B | 1761 | −14.419 | −39.506 | 68.069 | 1.00 | 182.59 | C |
| ATOM | 5576 | C | PRO | B | 1761 | −14.937 | −40.920 | 67.852 | 1.00 | 182.88 | C |
| ATOM | 5577 | O | PRO | B | 1761 | −14.861 | −41.763 | 68.752 | 1.00 | 182.85 | O |
| ATOM | 5578 | CB | PRO | B | 1761 | −13.001 | −39.372 | 67.522 | 1.00 | 182.40 | C |
| ATOM | 5579 | CG | PRO | B | 1761 | −12.159 | −39.776 | 68.630 | 1.00 | 182.17 | C |
| ATOM | 5580 | CD | PRO | B | 1761 | −12.843 | −39.262 | 69.872 | 1.00 | 182.30 | C |
| ATOM | 5581 | N | TYR | B | 1762 | −15.466 | −41.149 | 66.653 | 1.00 | 183.17 | N |
| ATOM | 5582 | CA | TYR | B | 1762 | −16.006 | −42.430 | 66.263 | 1.00 | 183.32 | C |
| ATOM | 5583 | C | TYR | B | 1762 | −14.867 | −43.414 | 66.094 | 1.00 | 183.51 | C |
| ATOM | 5584 | O | TYR | B | 1762 | −13.877 | −43.103 | 65.426 | 1.00 | 183.37 | O |
| ATOM | 5585 | CB | TYR | B | 1762 | −16.766 | −42.292 | 64.944 | 1.00 | 183.36 | C |
| ATOM | 5586 | CG | TYR | B | 1762 | −18.155 | −41.683 | 65.052 | 1.00 | 183.67 | C |
| ATOM | 5587 | CD1 | TYR | B | 1762 | −19.208 | −42.400 | 65.629 | 1.00 | 183.91 | C |
| ATOM | 5588 | CD2 | TYR | B | 1762 | −18.431 | −40.405 | 64.541 | 1.00 | 183.96 | C |
| ATOM | 5589 | CE1 | TYR | B | 1762 | −20.497 | −41.858 | 65.720 | 1.00 | 183.79 | C |
| ATOM | 5590 | CE2 | TYR | B | 1762 | −19.723 | −39.853 | 64.621 | 1.00 | 183.81 | C |
| ATOM | 5591 | CZ | TYR | B | 1762 | −20.749 | −40.590 | 65.217 | 1.00 | 183.74 | C |
| ATOM | 5592 | OH | TYR | B | 1762 | −22.026 | −40.077 | 65.323 | 1.00 | 183.29 | O |
| ATOM | 5593 | N | ILE | B | 1763 | −15.007 | −44.577 | 66.739 | 1.00 | 183.97 | N |
| ATOM | 5594 | CA | ILE | B | 1763 | −14.133 | −45.752 | 66.533 | 1.00 | 184.33 | C |
| ATOM | 5595 | C | ILE | B | 1763 | −14.967 | −46.885 | 65.904 | 1.00 | 184.60 | C |
| ATOM | 5596 | O | ILE | B | 1763 | −16.025 | −47.261 | 66.432 | 1.00 | 184.41 | O |
| ATOM | 5597 | CB | ILE | B | 1763 | −13.432 | −46.246 | 67.846 | 1.00 | 184.35 | C |
| ATOM | 5598 | CG1 | ILE | B | 1763 | −12.865 | −45.072 | 68.663 | 1.00 | 184.45 | C |
| ATOM | 5599 | CG2 | ILE | B | 1763 | −12.327 | −47.249 | 67.518 | 1.00 | 184.01 | C |
| ATOM | 5600 | CD1 | ILE | B | 1763 | −12.152 | −45.468 | 69.962 | 1.00 | 184.23 | C |
| ATOM | 5601 | N | ARG | B | 1764 | −14.477 | −47.417 | 64.780 | 1.00 | 184.92 | N |
| ATOM | 5602 | CA | ARG | B | 1764 | −15.303 | −48.196 | 63.847 | 1.00 | 185.43 | C |
| ATOM | 5603 | C | ARG | B | 1764 | −14.626 | −49.471 | 63.349 | 1.00 | 184.94 | C |
| ATOM | 5604 | O | ARG | B | 1764 | −13.439 | −49.461 | 63.031 | 1.00 | 185.02 | O |
| ATOM | 5605 | CB | ARG | B | 1764 | −15.668 | −47.332 | 62.628 | 1.00 | 185.52 | C |
| ATOM | 5606 | CG | ARG | B | 1764 | −15.845 | −45.830 | 62.916 | 1.00 | 186.62 | C |
| ATOM | 5607 | CD | ARG | B | 1764 | −15.955 | −44.990 | 61.642 | 1.00 | 186.79 | C |
| ATOM | 5608 | NE | ARG | B | 1764 | −17.270 | −45.144 | 61.019 | 1.00 | 189.51 | N |
| ATOM | 5609 | CZ | ARG | B | 1764 | −18.298 | −44.318 | 61.194 | 1.00 | 190.00 | C |
| ATOM | 5610 | NH1 | ARG | B | 1764 | −18.181 | −43.249 | 61.972 | 1.00 | 190.42 | N |
| ATOM | 5611 | NH2 | ARG | B | 1764 | −19.446 | −44.565 | 60.579 | 1.00 | 190.54 | N |
| ATOM | 5612 | N | ALA | B | 1765 | −15.394 | −50.554 | 63.246 | 1.00 | 184.63 | N |
| ATOM | 5613 | CA | ALA | B | 1765 | −14.857 | −51.834 | 62.792 | 1.00 | 184.45 | C |
| ATOM | 5614 | C | ALA | B | 1765 | −15.883 | −52.740 | 62.126 | 1.00 | 184.44 | C |
| ATOM | 5615 | O | ALA | B | 1765 | −17.078 | −52.644 | 62.395 | 1.00 | 184.35 | O |
| ATOM | 5616 | CB | ALA | B | 1765 | −14.230 | −52.559 | 63.950 | 1.00 | 184.53 | C |
| ATOM | 5617 | N | GLU | B | 1766 | −15.395 | −53.632 | 61.266 | 1.00 | 184.49 | N |
| ATOM | 5618 | CA | GLU | B | 1766 | −16.213 | −54.704 | 60.703 | 1.00 | 184.82 | C |
| ATOM | 5619 | C | GLU | B | 1766 | −16.156 | −55.967 | 61.580 | 1.00 | 184.76 | C |
| ATOM | 5620 | O | GLU | B | 1766 | −15.577 | −55.932 | 62.669 | 1.00 | 184.87 | O |
| ATOM | 5621 | CB | GLU | B | 1766 | −15.791 | −55.003 | 59.269 | 1.00 | 184.69 | C |
| ATOM | 5622 | CG | GLU | B | 1766 | −16.333 | −54.006 | 58.256 | 1.00 | 185.18 | C |
| ATOM | 5623 | CD | GLU | B | 1766 | −15.835 | −54.272 | 56.839 | 1.00 | 185.62 | C |
| ATOM | 5624 | OE1 | GLU | B | 1766 | −15.273 | −53.334 | 56.226 | 1.00 | 187.00 | O |
| ATOM | 5625 | OE2 | GLU | B | 1766 | −15.993 | −55.413 | 56.338 | 1.00 | 186.58 | O |
| ATOM | 5626 | N | VAL | B | 1767 | −16.767 | −57.066 | 61.119 | 1.00 | 184.70 | N |
| ATOM | 5627 | CA | VAL | B | 1767 | −16.868 | −58.299 | 61.920 | 1.00 | 184.58 | C |
| ATOM | 5628 | C | VAL | B | 1767 | −15.483 | −58.882 | 62.193 | 1.00 | 184.50 | C |
| ATOM | 5629 | O | VAL | B | 1767 | −14.932 | −58.678 | 63.268 | 1.00 | 184.48 | O |
| ATOM | 5630 | CB | VAL | B | 1767 | −17.854 | −59.364 | 61.309 | 1.00 | 184.64 | C |
| ATOM | 5631 | CG1 | VAL | B | 1767 | −17.830 | −60.689 | 62.095 | 1.00 | 184.83 | C |
| ATOM | 5632 | CG2 | VAL | B | 1767 | −19.278 | −58.830 | 61.274 | 1.00 | 184.49 | C |
| ATOM | 5633 | N | GLU | B | 1768 | −14.905 | −59.570 | 61.218 | 1.00 | 184.45 | N |
| ATOM | 5634 | CA | GLU | B | 1768 | −13.649 | −60.263 | 61.440 | 1.00 | 184.59 | C |
| ATOM | 5635 | C | GLU | B | 1768 | −12.467 | −59.287 | 61.392 | 1.00 | 184.51 | C |
| ATOM | 5636 | O | GLU | B | 1768 | −11.582 | −59.408 | 60.547 | 1.00 | 184.53 | O |
| ATOM | 5637 | CB | GLU | B | 1768 | −13.498 | −61.370 | 60.399 | 1.00 | 184.82 | C |
| ATOM | 5638 | CG | GLU | B | 1768 | −12.783 | −62.615 | 60.909 | 1.00 | 185.47 | C |
| ATOM | 5639 | CD | GLU | B | 1768 | −13.718 | −63.636 | 61.533 | 1.00 | 185.86 | C |
| ATOM | 5640 | OE1 | GLU | B | 1768 | −14.953 | −63.394 | 61.550 | 1.00 | 185.68 | O |
| ATOM | 5641 | OE2 | GLU | B | 1768 | −13.205 | −64.685 | 61.998 | 1.00 | 185.90 | O |
| ATOM | 5642 | N | ASP | B | 1769 | −12.451 | −58.331 | 62.318 | 1.00 | 184.54 | N |
| ATOM | 5643 | CA | ASP | B | 1769 | −11.554 | −57.166 | 62.232 | 1.00 | 184.61 | C |
| ATOM | 5644 | C | ASP | B | 1769 | −10.475 | −57.036 | 63.319 | 1.00 | 184.74 | C |
| ATOM | 5645 | O | ASP | B | 1769 | −10.268 | −57.932 | 64.142 | 1.00 | 184.80 | O |
| ATOM | 5646 | CB | ASP | B | 1769 | −12.379 | −55.868 | 62.191 | 1.00 | 184.51 | C |
| ATOM | 5647 | CG | ASP | B | 1769 | −12.446 | −55.243 | 60.809 | 1.00 | 184.13 | C |
| ATOM | 5648 | OD1 | ASP | B | 1769 | −12.417 | −55.979 | 59.803 | 1.00 | 184.46 | O |
| ATOM | 5649 | OD2 | ASP | B | 1769 | −12.541 | −54.002 | 60.730 | 1.00 | 183.48 | O |
| ATOM | 5650 | N | ASN | B | 1770 | −9.796 | −55.891 | 63.286 | 1.00 | 184.84 | N |
| ATOM | 5651 | CA | ASN | B | 1770 | −8.785 | −55.517 | 64.256 | 1.00 | 185.01 | C |
| ATOM | 5652 | C | ASN | B | 1770 | −8.979 | −54.090 | 64.722 | 1.00 | 185.21 | C |
| ATOM | 5653 | O | ASN | B | 1770 | −9.012 | −53.155 | 63.920 | 1.00 | 185.36 | O |
| ATOM | 5654 | CB | ASN | B | 1770 | −7.400 | −55.625 | 63.640 | 1.00 | 184.93 | C |
| ATOM | 5655 | CG | ASN | B | 1770 | −6.647 | −56.827 | 64.110 | 1.00 | 184.86 | C |
| ATOM | 5656 | OD1 | ASN | B | 1770 | −7.065 | −57.524 | 65.035 | 1.00 | 184.66 | O |
| ATOM | 5657 | ND2 | ASN | B | 1770 | −5.513 | −57.080 | 63.479 | 1.00 | 185.17 | N |
| ATOM | 5658 | N | ILE | B | 1771 | −9.093 | −53.925 | 66.027 | 1.00 | 185.40 | N |
| ATOM | 5659 | CA | ILE | B | 1771 | −9.220 | −52.607 | 66.609 | 1.00 | 185.74 | C |
| ATOM | 5660 | C | ILE | B | 1771 | −7.966 | −52.340 | 67.422 | 1.00 | 186.01 | C |
| ATOM | 5661 | O | ILE | B | 1771 | −7.484 | −53.225 | 68.122 | 1.00 | 186.16 | O |
| ATOM | 5662 | CB | ILE | B | 1771 | −10.495 | −52.513 | 67.484 | 1.00 | 185.84 | C |
| ATOM | 5663 | CG1 | ILE | B | 1771 | −11.749 | −52.601 | 66.601 | 1.00 | 185.82 | C |
| ATOM | 5664 | CG2 | ILE | B | 1771 | −10.511 | −51.232 | 68.317 | 1.00 | 186.10 | C |
| ATOM | 5665 | CD1 | ILE | B | 1771 | −13.071 | −52.600 | 67.358 | 1.00 | 185.61 | C |
| ATOM | 5666 | N | MET | B | 1772 | −7.432 | −51.127 | 67.314 | 1.00 | 186.38 | N |
| ATOM | 5667 | CA | MET | B | 1772 | −6.244 | −50.735 | 68.069 | 1.00 | 186.89 | C |
| ATOM | 5668 | C | MET | B | 1772 | −6.339 | −49.316 | 68.602 | 1.00 | 187.12 | C |
| ATOM | 5669 | O | MET | B | 1772 | −6.798 | −48.418 | 67.903 | 1.00 | 187.36 | O |
| ATOM | 5670 | CB | MET | B | 1772 | −5.013 | −50.849 | 67.189 | 1.00 | 186.77 | C |
| ATOM | 5671 | CG | MET | B | 1772 | −3.762 | −50.311 | 67.816 | 1.00 | 186.79 | C |
| ATOM | 5672 | SD | MET | B | 1772 | −2.429 | −50.524 | 66.657 | 1.00 | 187.35 | S |
| ATOM | 5673 | CE | MET | B | 1772 | −2.230 | −52.313 | 66.689 | 1.00 | 187.53 | C |
| ATOM | 5674 | N | VAL | B | 1773 | −5.891 | −49.113 | 69.836 | 1.00 | 187.42 | N |
| ATOM | 5675 | CA | VAL | B | 1773 | −5.875 | −47.776 | 70.418 | 1.00 | 187.75 | C |
| ATOM | 5676 | C | VAL | B | 1773 | −4.535 | −47.490 | 71.061 | 1.00 | 187.89 | C |
| ATOM | 5677 | O | VAL | B | 1773 | −4.151 | −48.123 | 72.051 | 1.00 | 188.10 | O |
| ATOM | 5678 | CB | VAL | B | 1773 | −7.041 | −47.558 | 71.425 | 1.00 | 187.82 | C |
| ATOM | 5679 | CG1 | VAL | B | 1773 | −6.660 | −46.570 | 72.526 | 1.00 | 187.81 | C |
| ATOM | 5680 | CG2 | VAL | B | 1773 | −8.298 | −47.094 | 70.691 | 1.00 | 188.03 | C |
| ATOM | 5681 | N | THR | B | 1774 | −3.820 | −46.542 | 70.477 | 1.00 | 187.91 | N |
| ATOM | 5682 | CA | THR | B | 1774 | −2.590 | −46.082 | 71.069 | 1.00 | 188.12 | C |
| ATOM | 5683 | C | THR | B | 1774 | −2.933 | −44.761 | 71.754 | 1.00 | 188.31 | C |
| ATOM | 5684 | O | THR | B | 1774 | −3.219 | −43.760 | 71.089 | 1.00 | 188.40 | O |
| ATOM | 5685 | CB | THR | B | 1774 | −1.479 | −45.975 | 70.018 | 1.00 | 188.13 | C |
| ATOM | 5686 | OG1 | THR | B | 1774 | −1.642 | −47.029 | 69.060 | 1.00 | 188.06 | O |
| ATOM | 5687 | CG2 | THR | B | 1774 | −0.097 | −46.102 | 70.670 | 1.00 | 188.22 | C |
| ATOM | 5688 | N | PHE | B | 1775 | −2.918 | −44.793 | 73.090 | 1.00 | 188.52 | N |
| ATOM | 5689 | CA | PHE | B | 1775 | −3.554 | −43.780 | 73.954 | 1.00 | 188.62 | C |
| ATOM | 5690 | C | PHE | B | 1775 | −2.558 | −43.019 | 74.833 | 1.00 | 188.85 | C |
| ATOM | 5691 | O | PHE | B | 1775 | −1.692 | −43.620 | 75.476 | 1.00 | 188.88 | O |
| ATOM | 5692 | CB | PHE | B | 1775 | −4.632 | −44.476 | 74.818 | 1.00 | 188.39 | C |
| ATOM | 5693 | CG | PHE | B | 1775 | −5.339 | −43.582 | 75.834 | 1.00 | 187.88 | C |
| ATOM | 5694 | CD1 | PHE | B | 1775 | −5.751 | −44.117 | 77.057 | 1.00 | 187.15 | C |
| ATOM | 5695 | CD2 | PHE | B | 1775 | −5.627 | −42.243 | 75.570 | 1.00 | 187.57 | C |
| ATOM | 5696 | CE1 | PHE | B | 1775 | −6.418 | −43.341 | 78.001 | 1.00 | 186.65 | C |
| ATOM | 5697 | CE2 | PHE | B | 1775 | −6.289 | −41.456 | 76.519 | 1.00 | 187.40 | C |
| ATOM | 5698 | CZ | PHE | B | 1775 | −6.686 | −42.012 | 77.733 | 1.00 | 187.16 | C |
| ATOM | 5699 | N | ARG | B | 1776 | −2.693 | −41.693 | 74.844 | 1.00 | 189.10 | N |
| ATOM | 5700 | CA | ARG | B | 1776 | −1.969 | −40.849 | 75.785 | 1.00 | 189.37 | C |
| ATOM | 5701 | C | ARG | B | 1776 | −2.901 | −40.008 | 76.647 | 1.00 | 189.67 | C |
| ATOM | 5702 | O | ARG | B | 1776 | −3.838 | −39.376 | 76.147 | 1.00 | 189.57 | O |
| ATOM | 5703 | CB | ARG | B | 1776 | −0.975 | −39.945 | 75.063 | 1.00 | 189.29 | C |
| ATOM | 5704 | CG | ARG | B | 1776 | 0.426 | −40.487 | 75.041 | 1.00 | 188.94 | C |
| ATOM | 5705 | CD | ARG | B | 1776 | 1.373 | −39.431 | 74.562 | 1.00 | 188.55 | C |
| ATOM | 5706 | NE | ARG | B | 1776 | 2.690 | −39.981 | 74.273 | 1.00 | 188.68 | N |
| ATOM | 5707 | CZ | ARG | B | 1776 | 3.627 | −39.350 | 73.571 | 1.00 | 188.97 | C |
| ATOM | 5708 | NH1 | ARG | B | 1776 | 3.397 | −38.141 | 73.070 | 1.00 | 189.08 | N |
| ATOM | 5709 | NH2 | ARG | B | 1776 | 4.801 | −39.931 | 73.364 | 1.00 | 189.11 | N |
| ATOM | 5710 | N | ASN | B | 1777 | −2.635 | −40.018 | 77.950 | 1.00 | 190.10 | N |
| ATOM | 5711 | CA | ASN | B | 1777 | −3.330 | −39.141 | 78.886 | 1.00 | 190.53 | C |
| ATOM | 5712 | C | ASN | B | 1777 | −2.573 | −37.829 | 79.053 | 1.00 | 190.94 | C |
| ATOM | 5713 | O | ASN | B | 1777 | −1.341 | −37.808 | 79.181 | 1.00 | 191.09 | O |
| ATOM | 5714 | CB | ASN | B | 1777 | −3.529 | −39.826 | 80.248 | 1.00 | 190.43 | C |
| ATOM | 5715 | CG | ASN | B | 1777 | −4.251 | −38.935 | 81.270 | 1.00 | 189.99 | C |
| ATOM | 5716 | OD1 | ASN | B | 1777 | −4.914 | −37.951 | 80.916 | 1.00 | 188.90 | O |
| ATOM | 5717 | ND2 | ASN | B | 1777 | −4.122 | −39.290 | 82.547 | 1.00 | 189.48 | N |
| ATOM | 5718 | N | GLN | B | 1778 | −3.326 | −36.735 | 79.031 | 1.00 | 191.29 | N |
| ATOM | 5719 | CA | GLN | B | 1778 | −2.788 | −35.412 | 79.304 | 1.00 | 191.60 | C |
| ATOM | 5720 | C | GLN | B | 1778 | −3.791 | −34.761 | 80.255 | 1.00 | 191.71 | C |
| ATOM | 5721 | O | GLN | B | 1778 | −4.799 | −34.213 | 79.811 | 1.00 | 191.91 | O |
| ATOM | 5722 | CB | GLN | B | 1778 | −2.623 | −34.612 | 77.995 | 1.00 | 191.63 | C |
| ATOM | 5723 | CG | GLN | B | 1778 | −1.549 | −35.168 | 77.021 | 1.00 | 191.79 | C |
| ATOM | 5724 | CD | GLN | B | 1778 | −1.894 | −35.011 | 75.524 | 1.00 | 191.66 | C |
| ATOM | 5725 | OE1 | GLN | B | 1778 | −1.068 | −35.313 | 74.655 | 1.00 | 191.29 | O |
| ATOM | 5726 | NE2 | GLN | B | 1778 | −3.110 | −34.551 | 75.226 | 1.00 | 191.59 | N |
| ATOM | 5727 | N | ALA | B | 1779 | −3.535 | −34.865 | 81.562 | 1.00 | 191.75 | N |
| ATOM | 5728 | CA | ALA | B | 1779 | −4.453 | −34.354 | 82.597 | 1.00 | 191.77 | C |
| ATOM | 5729 | C | ALA | B | 1779 | −3.909 | −34.512 | 84.027 | 1.00 | 191.82 | C |
| ATOM | 5730 | O | ALA | B | 1779 | −2.754 | −34.905 | 84.219 | 1.00 | 191.93 | O |
| ATOM | 5731 | CB | ALA | B | 1779 | −5.825 | −35.029 | 82.475 | 1.00 | 191.75 | C |
| ATOM | 5732 | N | SER | B | 1780 | −4.746 | −34.191 | 85.019 | 1.00 | 191.77 | N |
| ATOM | 5733 | CA | SER | B | 1780 | −4.435 | −34.416 | 86.438 | 1.00 | 191.65 | C |
| ATOM | 5734 | C | SER | B | 1780 | −4.681 | −35.880 | 86.815 | 1.00 | 191.55 | C |
| ATOM | 5735 | O | SER | B | 1780 | −3.810 | −36.737 | 86.617 | 1.00 | 191.48 | O |
| ATOM | 5736 | CB | SER | B | 1780 | −5.265 | −33.488 | 87.341 | 1.00 | 191.64 | C |
| ATOM | 5737 | OG | SER | B | 1780 | −4.687 | −32.201 | 87.461 | 1.00 | 191.57 | O |
| ATOM | 5738 | N | ARG | B | 1781 | −5.871 | −36.147 | 87.362 | 1.00 | 191.49 | N |
| ATOM | 5739 | CA | ARG | B | 1781 | −6.315 | −37.497 | 87.714 | 1.00 | 191.41 | C |
| ATOM | 5740 | C | ARG | B | 1781 | −6.101 | −38.407 | 86.493 | 1.00 | 191.90 | C |
| ATOM | 5741 | O | ARG | B | 1781 | −6.673 | −38.153 | 85.432 | 1.00 | 192.08 | O |
| ATOM | 5742 | CB | ARG | B | 1781 | −7.796 | −37.474 | 88.158 | 1.00 | 191.02 | C |
| ATOM | 5743 | CG | ARG | B | 1781 | −8.285 | −38.694 | 88.947 | 1.00 | 189.00 | C |
| ATOM | 5744 | CD | ARG | B | 1781 | −9.785 | −38.870 | 88.789 | 1.00 | 185.54 | C |
| ATOM | 5745 | NE | ARG | B | 1781 | −10.557 | −38.027 | 89.696 | 1.00 | 182.92 | N |
| ATOM | 5746 | CZ | ARG | B | 1781 | −11.723 | −37.461 | 89.397 | 1.00 | 181.04 | C |
| ATOM | 5747 | NH1 | ARG | B | 1781 | −12.264 | −37.606 | 88.200 | 1.00 | 179.66 | N |
| ATOM | 5748 | NH2 | ARG | B | 1781 | −12.346 | −36.729 | 90.301 | 1.00 | 180.13 | N |
| ATOM | 5749 | N | PRO | B | 1782 | −5.229 | −39.432 | 86.619 | 1.00 | 192.19 | N |
| ATOM | 5750 | CA | PRO | B | 1782 | −5.027 | −40.400 | 85.533 | 1.00 | 192.36 | C |
| ATOM | 5751 | C | PRO | B | 1782 | −6.331 | −41.113 | 85.137 | 1.00 | 192.50 | C |
| ATOM | 5752 | O | PRO | B | 1782 | −7.112 | −41.496 | 86.014 | 1.00 | 192.58 | O |
| ATOM | 5753 | CB | PRO | B | 1782 | −4.041 | −41.401 | 86.141 | 1.00 | 192.42 | C |
| ATOM | 5754 | CG | PRO | B | 1782 | −3.345 | −40.644 | 87.226 | 1.00 | 192.33 | C |
| ATOM | 5755 | CD | PRO | B | 1782 | −4.372 | −39.725 | 87.784 | 1.00 | 192.23 | C |
| ATOM | 5756 | N | TYR | B | 1783 | −6.563 | −41.271 | 83.832 | 1.00 | 192.57 | N |
| ATOM | 5757 | CA | TYR | B | 1783 | −7.778 | −41.928 | 83.322 | 1.00 | 192.61 | C |
| ATOM | 5758 | C | TYR | B | 1783 | −7.437 | −43.148 | 82.450 | 1.00 | 192.69 | C |
| ATOM | 5759 | O | TYR | B | 1783 | −6.289 | −43.594 | 82.443 | 1.00 | 192.65 | O |
| ATOM | 5760 | CB | TYR | B | 1783 | −8.666 | −40.932 | 82.555 | 1.00 | 192.58 | C |
| ATOM | 5761 | CG | TYR | B | 1783 | −9.003 | −39.637 | 83.283 | 1.00 | 192.40 | C |
| ATOM | 5762 | CD1 | TYR | B | 1783 | −9.524 | −39.644 | 84.580 | 1.00 | 192.49 | C |
| ATOM | 5763 | CD2 | TYR | B | 1783 | −8.830 | −38.402 | 82.654 | 1.00 | 192.23 | C |
| ATOM | 5764 | CE1 | TYR | B | 1783 | −9.840 | −38.444 | 85.241 | 1.00 | 192.78 | C |
| ATOM | 5765 | CE2 | TYR | B | 1783 | −9.142 | −37.202 | 83.301 | 1.00 | 192.37 | C |
| ATOM | 5766 | CZ | TYR | B | 1783 | −9.646 | −37.226 | 84.592 | 1.00 | 192.56 | C |
| ATOM | 5767 | OH | TYR | B | 1783 | −9.955 | −36.037 | 85.230 | 1.00 | 192.36 | O |
| ATOM | 5768 | N | SER | B | 1784 | −8.427 | −43.684 | 81.727 | 1.00 | 192.87 | N |
| ATOM | 5769 | CA | SER | B | 1784 | −8.231 | −44.888 | 80.898 | 1.00 | 193.19 | C |
| ATOM | 5770 | C | SER | B | 1784 | −9.284 | −45.113 | 79.803 | 1.00 | 193.36 | C |
| ATOM | 5771 | O | SER | B | 1784 | −10.384 | −44.563 | 79.867 | 1.00 | 193.43 | O |
| ATOM | 5772 | CB | SER | B | 1784 | −8.162 | −46.138 | 81.787 | 1.00 | 193.24 | C |
| ATOM | 5773 | OG | SER | B | 1784 | −8.281 | −47.326 | 81.018 | 1.00 | 193.21 | O |
| ATOM | 5774 | N | PHE | B | 1785 | −8.920 | −45.929 | 78.806 | 1.00 | 193.49 | N |
| ATOM | 5775 | CA | PHE | B | 1785 | −9.860 | −46.477 | 77.811 | 1.00 | 193.56 | C |
| ATOM | 5776 | C | PHE | B | 1785 | −10.501 | −47.731 | 78.379 | 1.00 | 193.31 | C |
| ATOM | 5777 | O | PHE | B | 1785 | −9.841 | −48.504 | 79.077 | 1.00 | 193.20 | O |
| ATOM | 5778 | CB | PHE | B | 1785 | −9.155 | −46.820 | 76.481 | 1.00 | 193.73 | C |
| ATOM | 5779 | CG | PHE | B | 1785 | −8.052 | −47.870 | 76.609 | 1.00 | 194.47 | C |
| ATOM | 5780 | CD1 | PHE | B | 1785 | −6.850 | −47.584 | 77.273 | 1.00 | 194.83 | C |
| ATOM | 5781 | CD2 | PHE | B | 1785 | −8.208 | −49.137 | 76.049 | 1.00 | 194.85 | C |
| ATOM | 5782 | CE1 | PHE | B | 1785 | −5.830 | −48.547 | 77.386 | 1.00 | 194.51 | C |
| ATOM | 5783 | CE2 | PHE | B | 1785 | −7.190 | −50.105 | 76.159 | 1.00 | 194.34 | C |
| ATOM | 5784 | CZ | PHE | B | 1785 | −6.003 | −49.803 | 76.828 | 1.00 | 194.17 | C |
| ATOM | 5785 | N | TYR | B | 1786 | −11.787 | −47.912 | 78.093 | 1.00 | 193.10 | N |
| ATOM | 5786 | CA | TYR | B | 1786 | −12.514 | −49.127 | 78.461 | 1.00 | 192.98 | C |
| ATOM | 5787 | C | TYR | B | 1786 | −13.831 | −49.201 | 77.724 | 1.00 | 193.10 | C |
| ATOM | 5788 | O | TYR | B | 1786 | −14.569 | −48.218 | 77.661 | 1.00 | 193.03 | O |
| ATOM | 5789 | CB | TYR | B | 1786 | −12.771 | −49.230 | 79.982 | 1.00 | 192.76 | C |
| ATOM | 5790 | CG | TYR | B | 1786 | −13.877 | −50.208 | 80.356 | 1.00 | 192.26 | C |
| ATOM | 5791 | CD1 | TYR | B | 1786 | −15.186 | −49.765 | 80.528 | 1.00 | 192.08 | C |
| ATOM | 5792 | CD2 | TYR | B | 1786 | −13.621 | −51.572 | 80.515 | 1.00 | 191.87 | C |
| ATOM | 5793 | CE1 | TYR | B | 1786 | −16.214 | −50.645 | 80.853 | 1.00 | 192.31 | C |
| ATOM | 5794 | CE2 | TYR | B | 1786 | −14.649 | −52.463 | 80.842 | 1.00 | 192.06 | C |
| ATOM | 5795 | CZ | TYR | B | 1786 | −15.945 | −51.986 | 81.005 | 1.00 | 192.27 | C |
| ATOM | 5796 | OH | TYR | B | 1786 | −16.987 | −52.827 | 81.321 | 1.00 | 192.23 | O |
| ATOM | 5797 | N | SER | B | 1787 | −14.109 | −50.375 | 77.165 | 1.00 | 193.35 | N |
| ATOM | 5798 | CA | SER | B | 1787 | −15.456 | −50.735 | 76.751 | 1.00 | 193.69 | C |
| ATOM | 5799 | C | SER | B | 1787 | −15.690 | −52.209 | 77.056 | 1.00 | 194.07 | C |
| ATOM | 5800 | O | SER | B | 1787 | −14.951 | −52.825 | 77.826 | 1.00 | 194.21 | O |
| ATOM | 5801 | CB | SER | B | 1787 | −15.684 | −50.451 | 75.270 | 1.00 | 193.45 | C |
| ATOM | 5802 | OG | SER | B | 1787 | −15.918 | −51.649 | 74.558 | 1.00 | 193.32 | O |
| ATOM | 5803 | N | SER | B | 1788 | −16.716 | −52.773 | 76.439 | 1.00 | 194.53 | N |
| ATOM | 5804 | CA | SER | B | 1788 | −17.059 | −54.160 | 76.668 | 1.00 | 195.07 | C |
| ATOM | 5805 | C | SER | B | 1788 | −16.057 | −55.138 | 76.030 | 1.00 | 195.42 | C |
| ATOM | 5806 | O | SER | B | 1788 | −15.202 | −55.653 | 76.755 | 1.00 | 195.60 | O |
| ATOM | 5807 | CB | SER | B | 1788 | −18.504 | −54.399 | 76.268 | 1.00 | 195.13 | C |
| ATOM | 5808 | OG | SER | B | 1788 | −19.301 | −53.391 | 76.869 | 1.00 | 195.23 | O |
| ATOM | 5809 | N | LEU | B | 1789 | −16.120 | −55.394 | 74.716 | 1.00 | 195.69 | N |
| ATOM | 5810 | CA | LEU | B | 1789 | −15.061 | −56.213 | 74.091 | 1.00 | 196.02 | C |
| ATOM | 5811 | C | LEU | B | 1789 | −13.754 | −55.467 | 74.236 | 1.00 | 196.28 | C |
| ATOM | 5812 | O | LEU | B | 1789 | −13.394 | −54.617 | 73.433 | 1.00 | 196.25 | O |
| ATOM | 5813 | CB | LEU | B | 1789 | −15.306 | −56.594 | 72.612 | 1.00 | 195.92 | C |
| ATOM | 5814 | CG | LEU | B | 1789 | −15.724 | −58.005 | 72.127 | 1.00 | 195.77 | C |
| ATOM | 5815 | CD1 | LEU | B | 1789 | −15.491 | −58.174 | 70.610 | 1.00 | 194.04 | C |
| ATOM | 5816 | CD2 | LEU | B | 1789 | −15.052 | −59.147 | 72.899 | 1.00 | 195.86 | C |
| ATOM | 5817 | N | ILE | B | 1790 | −13.075 | −55.767 | 75.322 | 1.00 | 196.73 | N |
| ATOM | 5818 | CA | ILE | B | 1790 | −11.755 | −55.268 | 75.532 | 1.00 | 197.24 | C |
| ATOM | 5819 | C | ILE | B | 1790 | −10.938 | −56.536 | 75.763 | 1.00 | 197.62 | C |
| ATOM | 5820 | O | ILE | B | 1790 | −10.079 | −56.602 | 76.638 | 1.00 | 197.77 | O |
| ATOM | 5821 | CB | ILE | B | 1790 | −11.739 | −54.217 | 76.679 | 1.00 | 197.25 | C |
| ATOM | 5822 | CG1 | ILE | B | 1790 | −10.414 | −53.450 | 76.677 | 1.00 | 197.55 | C |
| ATOM | 5823 | CG2 | ILE | B | 1790 | −12.108 | −54.843 | 78.051 | 1.00 | 197.12 | C |
| ATOM | 5824 | CD1 | ILE | B | 1790 | −10.517 | −52.035 | 77.178 | 1.00 | 198.01 | C |
| ATOM | 5825 | N | SER | B | 1791 | −11.236 | −57.540 | 74.936 | 1.00 | 198.02 | N |
| ATOM | 5826 | CA | SER | B | 1791 | −10.723 | −58.902 | 75.076 | 1.00 | 198.54 | C |
| ATOM | 5827 | C | SER | B | 1791 | −9.242 | −58.998 | 75.419 | 1.00 | 198.95 | C |
| ATOM | 5828 | O | SER | B | 1791 | −8.400 | −59.148 | 74.532 | 1.00 | 199.07 | O |
| ATOM | 5829 | CB | SER | B | 1791 | −11.006 | −59.699 | 73.804 | 1.00 | 198.50 | C |
| ATOM | 5830 | OG | SER | B | 1791 | −12.397 | −59.801 | 73.584 | 1.00 | 198.58 | O |
| ATOM | 5831 | N | TYR | B | 1792 | −8.934 | −58.916 | 76.713 | 1.00 | 199.49 | N |
| ATOM | 5832 | CA | TYR | B | 1792 | −7.564 | −59.083 | 77.193 | 1.00 | 200.02 | C |
| ATOM | 5833 | C | TYR | B | 1792 | −7.161 | −60.544 | 76.986 | 1.00 | 200.37 | C |
| ATOM | 5834 | O | TYR | B | 1792 | −7.955 | −61.347 | 76.483 | 1.00 | 200.19 | O |
| ATOM | 5835 | CB | TYR | B | 1792 | −7.409 | −58.670 | 78.671 | 1.00 | 200.05 | C |
| ATOM | 5836 | CG | TYR | B | 1792 | −7.835 | −57.243 | 79.051 | 1.00 | 200.23 | C |
| ATOM | 5837 | CD1 | TYR | B | 1792 | −8.575 | −57.012 | 80.220 | 1.00 | 200.46 | C |
| ATOM | 5838 | CD2 | TYR | B | 1792 | −7.482 | −56.130 | 78.269 | 1.00 | 199.94 | C |
| ATOM | 5839 | CE1 | TYR | B | 1792 | −8.967 | −55.722 | 80.595 | 1.00 | 200.29 | C |
| ATOM | 5840 | CE2 | TYR | B | 1792 | −7.873 | −54.831 | 78.638 | 1.00 | 199.66 | C |
| ATOM | 5841 | CZ | TYR | B | 1792 | −8.617 | −54.641 | 79.801 | 1.00 | 199.99 | C |
| ATOM | 5842 | OH | TYR | B | 1792 | −9.016 | −53.380 | 80.184 | 1.00 | 199.89 | O |
| ATOM | 5843 | N | GLU | B | 1793 | −5.939 | −60.896 | 77.375 | 1.00 | 201.01 | N |
| ATOM | 5844 | CA | GLU | B | 1793 | −5.399 | −62.181 | 76.961 | 1.00 | 201.76 | C |
| ATOM | 5845 | C | GLU | B | 1793 | −4.656 | −63.006 | 78.008 | 1.00 | 202.40 | C |
| ATOM | 5846 | O | GLU | B | 1793 | −3.911 | −62.466 | 78.828 | 1.00 | 202.56 | O |
| ATOM | 5847 | CB | GLU | B | 1793 | −4.531 | −62.000 | 75.709 | 1.00 | 201.65 | C |
| ATOM | 5848 | CG | GLU | B | 1793 | −4.724 | −63.106 | 74.710 | 1.00 | 201.71 | C |
| ATOM | 5849 | CD | GLU | B | 1793 | −6.093 | −63.738 | 74.856 | 1.00 | 202.29 | C |
| ATOM | 5850 | OE1 | GLU | B | 1793 | −7.098 | −63.092 | 74.482 | 1.00 | 202.73 | O |
| ATOM | 5851 | OE2 | GLU | B | 1793 | −6.164 | −64.873 | 75.373 | 1.00 | 202.48 | O |
| ATOM | 5852 | N | GLU | B | 1794 | −4.892 | −64.321 | 77.964 | 1.00 | 203.14 | N |
| ATOM | 5853 | CA | GLU | B | 1794 | −4.058 | −65.336 | 78.627 | 1.00 | 203.95 | C |
| ATOM | 5854 | C | GLU | B | 1794 | −3.987 | −65.230 | 80.149 | 1.00 | 204.43 | C |
| ATOM | 5855 | O | GLU | B | 1794 | −5.012 | −65.155 | 80.822 | 1.00 | 204.44 | O |
| ATOM | 5856 | CB | GLU | B | 1794 | −2.635 | −65.323 | 78.048 | 1.00 | 203.95 | C |
| ATOM | 5857 | CG | GLU | B | 1794 | −2.543 | −65.488 | 76.531 | 1.00 | 204.36 | C |
| ATOM | 5858 | CD | GLU | B | 1794 | −1.209 | −65.012 | 75.958 | 1.00 | 204.23 | C |
| ATOM | 5859 | OE1 | GLU | B | 1794 | −0.340 | −64.563 | 76.741 | 1.00 | 204.20 | O |
| ATOM | 5860 | OE2 | GLU | B | 1794 | −1.033 | −65.083 | 74.719 | 1.00 | 204.57 | O |
| ATOM | 5861 | N | ASP | B | 1795 | −2.755 | −65.242 | 80.665 | 1.00 | 205.25 | N |
| ATOM | 5862 | CA | ASP | B | 1795 | −2.448 | −65.152 | 82.098 | 1.00 | 206.08 | C |
| ATOM | 5863 | C | ASP | B | 1795 | −0.991 | −64.702 | 82.314 | 1.00 | 206.69 | C |
| ATOM | 5864 | O | ASP | B | 1795 | −0.287 | −64.401 | 81.342 | 1.00 | 206.82 | O |
| ATOM | 5865 | CB | ASP | B | 1795 | −2.709 | −66.498 | 82.798 | 1.00 | 206.05 | C |
| ATOM | 5866 | CG | ASP | B | 1795 | −1.655 | −67.554 | 82.473 | 1.00 | 205.79 | C |
| ATOM | 5867 | OD1 | ASP | B | 1795 | −1.284 | −67.701 | 81.288 | 1.00 | 205.33 | O |
| ATOM | 5868 | OD2 | ASP | B | 1795 | −1.208 | −68.247 | 83.414 | 1.00 | 205.55 | O |
| ATOM | 5869 | N | GLN | B | 1796 | −0.544 | −64.667 | 83.576 | 1.00 | 207.40 | N |
| ATOM | 5870 | CA | GLN | B | 1796 | 0.831 | −64.254 | 83.930 | 1.00 | 208.04 | C |
| ATOM | 5871 | C | GLN | B | 1796 | 1.307 | −64.745 | 85.321 | 1.00 | 208.33 | C |
| ATOM | 5872 | O | GLN | B | 1796 | 0.491 | −65.100 | 86.180 | 1.00 | 208.45 | O |
| ATOM | 5873 | CB | GLN | B | 1796 | 0.964 | −62.721 | 83.841 | 1.00 | 208.11 | C |
| ATOM | 5874 | CG | GLN | B | 1796 | 2.332 | −62.220 | 83.360 | 1.00 | 208.41 | C |
| ATOM | 5875 | CD | GLN | B | 1796 | 2.516 | −62.344 | 81.851 | 1.00 | 208.59 | C |
| ATOM | 5876 | OE1 | GLN | B | 1796 | 1.728 | −61.804 | 81.069 | 1.00 | 208.66 | O |
| ATOM | 5877 | NE2 | GLN | B | 1796 | 3.567 | −63.049 | 81.438 | 1.00 | 208.51 | N |
| ATOM | 5878 | N | ARG | B | 1797 | 2.630 | −64.775 | 85.521 | 1.00 | 208.58 | N |
| ATOM | 5879 | CA | ARG | B | 1797 | 3.235 | −64.943 | 86.845 | 1.00 | 208.74 | C |
| ATOM | 5880 | C | ARG | B | 1797 | 2.847 | −63.712 | 87.672 | 1.00 | 208.86 | C |
| ATOM | 5881 | O | ARG | B | 1797 | 3.521 | −62.679 | 87.632 | 1.00 | 208.87 | O |
| ATOM | 5882 | CB | ARG | B | 1797 | 4.758 | −65.097 | 86.715 | 1.00 | 208.73 | C |
| ATOM | 5883 | CG | ARG | B | 1797 | 5.556 | −65.103 | 88.022 | 1.00 | 209.07 | C |
| ATOM | 5884 | CD | ARG | B | 1797 | 5.593 | −66.471 | 88.691 | 1.00 | 209.62 | C |
| ATOM | 5885 | NE | ARG | B | 1797 | 4.556 | −66.613 | 89.712 | 1.00 | 210.01 | N |
| ATOM | 5886 | CZ | ARG | B | 1797 | 3.389 | −67.228 | 89.532 | 1.00 | 210.22 | C |
| ATOM | 5887 | NH1 | ARG | B | 1797 | 3.085 | −67.775 | 88.359 | 1.00 | 210.40 | N |
| ATOM | 5888 | NH2 | ARG | B | 1797 | 2.520 | −67.298 | 90.533 | 1.00 | 210.16 | N |
| ATOM | 5889 | N | GLN | B | 1798 | 1.739 | −63.840 | 88.401 | 1.00 | 209.04 | N |
| ATOM | 5890 | CA | GLN | B | 1798 | 1.034 | −62.693 | 88.980 | 1.00 | 209.22 | C |
| ATOM | 5891 | C | GLN | B | 1798 | 0.640 | −62.940 | 90.446 | 1.00 | 209.55 | C |
| ATOM | 5892 | O | GLN | B | 1798 | 0.923 | −64.007 | 90.996 | 1.00 | 209.62 | O |
| ATOM | 5893 | CB | GLN | B | 1798 | −0.207 | −62.387 | 88.130 | 1.00 | 209.03 | C |
| ATOM | 5894 | CG | GLN | B | 1798 | −0.480 | −60.913 | 87.894 | 1.00 | 208.53 | C |
| ATOM | 5895 | CD | GLN | B | 1798 | 0.306 | −60.330 | 86.736 | 1.00 | 208.12 | C |
| ATOM | 5896 | OE1 | GLN | B | 1798 | 1.537 | −60.292 | 86.756 | 1.00 | 207.82 | O |
| ATOM | 5897 | NE2 | GLN | B | 1798 | −0.408 | −59.853 | 85.724 | 1.00 | 208.06 | N |
| ATOM | 5898 | N | GLY | B | 1799 | −0.003 | −61.950 | 91.071 | 1.00 | 209.90 | N |
| ATOM | 5899 | CA | GLY | B | 1799 | −0.460 | −62.053 | 92.461 | 1.00 | 210.35 | C |
| ATOM | 5900 | C | GLY | B | 1799 | −1.965 | −61.884 | 92.597 | 1.00 | 210.71 | C |
| ATOM | 5901 | O | GLY | B | 1799 | −2.437 | −60.867 | 93.114 | 1.00 | 210.75 | O |
| ATOM | 5902 | N | ALA | B | 1800 | −2.702 | −62.900 | 92.138 | 1.00 | 211.03 | N |
| ATOM | 5903 | CA | ALA | B | 1800 | −4.179 | −62.916 | 92.063 | 1.00 | 211.35 | C |
| ATOM | 5904 | C | ALA | B | 1800 | −4.769 | −62.140 | 90.858 | 1.00 | 211.62 | C |
| ATOM | 5905 | O | ALA | B | 1800 | −5.934 | −61.730 | 90.880 | 1.00 | 211.71 | O |
| ATOM | 5906 | CB | ALA | B | 1800 | −4.826 | −62.490 | 93.407 | 1.00 | 211.26 | C |
| ATOM | 5907 | N | GLU | B | 1801 | −3.954 | −61.968 | 89.812 | 1.00 | 211.92 | N |
| ATOM | 5908 | CA | GLU | B | 1801 | −4.344 | −61.356 | 88.511 | 1.00 | 212.14 | C |
| ATOM | 5909 | C | GLU | B | 1801 | −4.702 | −59.844 | 88.513 | 1.00 | 212.19 | C |
| ATOM | 5910 | O | GLU | B | 1801 | −5.864 | −59.476 | 88.271 | 1.00 | 212.14 | O |
| ATOM | 5911 | CB | GLU | B | 1801 | −5.436 | −62.188 | 87.792 | 1.00 | 212.22 | C |
| ATOM | 5912 | CG | GLU | B | 1801 | −5.531 | −61.979 | 86.262 | 1.00 | 212.17 | C |
| ATOM | 5913 | CD | GLU | B | 1801 | −4.657 | −62.943 | 85.461 | 1.00 | 211.93 | C |
| ATOM | 5914 | OE1 | GLU | B | 1801 | −4.800 | −64.175 | 85.637 | 1.00 | 211.69 | O |
| ATOM | 5915 | OE2 | GLU | B | 1801 | −3.838 | −62.469 | 84.645 | 1.00 | 211.64 | O |
| ATOM | 5916 | N | PRO | B | 1802 | −3.705 | −58.967 | 88.792 | 1.00 | 212.20 | N |
| ATOM | 5917 | CA | PRO | B | 1802 | −3.842 | −57.544 | 88.462 | 1.00 | 212.12 | C |
| ATOM | 5918 | C | PRO | B | 1802 | −4.169 | −57.319 | 86.980 | 1.00 | 211.97 | C |
| ATOM | 5919 | O | PRO | B | 1802 | −3.269 | −57.205 | 86.140 | 1.00 | 211.85 | O |
| ATOM | 5920 | CB | PRO | B | 1802 | −2.461 | −56.971 | 88.811 | 1.00 | 212.20 | C |
| ATOM | 5921 | CG | PRO | B | 1802 | −1.940 | −57.873 | 89.870 | 1.00 | 212.12 | C |
| ATOM | 5922 | CD | PRO | B | 1802 | −2.431 | −59.238 | 89.489 | 1.00 | 212.16 | C |
| ATOM | 5923 | N | ARG | B | 1803 | −5.462 | −57.279 | 86.678 | 1.00 | 211.85 | N |
| ATOM | 5924 | CA | ARG | B | 1803 | −5.950 | −56.992 | 85.336 | 1.00 | 211.81 | C |
| ATOM | 5925 | C | ARG | B | 1803 | −6.374 | −55.516 | 85.252 | 1.00 | 211.69 | C |
| ATOM | 5926 | O | ARG | B | 1803 | −7.200 | −55.124 | 84.410 | 1.00 | 211.67 | O |
| ATOM | 5927 | CB | ARG | B | 1803 | −7.107 | −57.940 | 84.995 | 1.00 | 211.84 | C |
| ATOM | 5928 | CG | ARG | B | 1803 | −7.481 | −57.968 | 83.522 | 1.00 | 211.93 | C |
| ATOM | 5929 | CD | ARG | B | 1803 | −7.785 | −59.377 | 83.057 | 1.00 | 212.02 | C |
| ATOM | 5930 | NE | ARG | B | 1803 | −6.646 | −60.279 | 83.238 | 1.00 | 211.87 | N |
| ATOM | 5931 | CZ | ARG | B | 1803 | −5.654 | −60.435 | 82.364 | 1.00 | 211.79 | C |
| ATOM | 5932 | NH1 | ARG | B | 1803 | −5.635 | −59.750 | 81.227 | 1.00 | 211.54 | N |
| ATOM | 5933 | NH2 | ARG | B | 1803 | −4.670 | −61.282 | 82.629 | 1.00 | 211.75 | N |
| ATOM | 5934 | N | LYS | B | 1804 | −5.778 | −54.704 | 86.129 | 1.00 | 211.42 | N |
| ATOM | 5935 | CA | LYS | B | 1804 | −6.171 | −53.310 | 86.313 | 1.00 | 211.14 | C |
| ATOM | 5936 | C | LYS | B | 1804 | −5.649 | −52.410 | 85.191 | 1.00 | 211.03 | C |
| ATOM | 5937 | O | LYS | B | 1804 | −4.442 | −52.353 | 84.930 | 1.00 | 210.94 | O |
| ATOM | 5938 | CB | LYS | B | 1804 | −5.720 | −52.791 | 87.691 | 1.00 | 211.02 | C |
| ATOM | 5939 | CG | LYS | B | 1804 | −6.155 | −53.646 | 88.894 | 1.00 | 210.85 | C |
| ATOM | 5940 | CD | LYS | B | 1804 | −7.640 | −53.498 | 89.238 | 1.00 | 210.53 | C |
| ATOM | 5941 | CE | LYS | B | 1804 | −8.048 | −54.505 | 90.308 | 1.00 | 210.46 | C |
| ATOM | 5942 | NZ | LYS | B | 1804 | −9.522 | −54.596 | 90.479 | 1.00 | 210.02 | N |
| ATOM | 5943 | N | ASN | B | 1805 | −6.574 | −51.722 | 84.523 | 1.00 | 210.90 | N |
| ATOM | 5944 | CA | ASN | B | 1805 | −6.220 | −50.751 | 83.497 | 1.00 | 210.80 | C |
| ATOM | 5945 | C | ASN | B | 1805 | −6.123 | −49.339 | 84.067 | 1.00 | 210.57 | C |
| ATOM | 5946 | O | ASN | B | 1805 | −7.133 | −48.728 | 84.441 | 1.00 | 210.41 | O |
| ATOM | 5947 | CB | ASN | B | 1805 | −7.205 | −50.797 | 82.327 | 1.00 | 210.99 | C |
| ATOM | 5948 | CG | ASN | B | 1805 | −6.590 | −50.292 | 81.021 | 1.00 | 211.63 | C |
| ATOM | 5949 | OD1 | ASN | B | 1805 | −5.387 | −50.451 | 80.773 | 1.00 | 212.28 | O |
| ATOM | 5950 | ND2 | ASN | B | 1805 | −7.421 | −49.689 | 80.174 | 1.00 | 212.05 | N |
| ATOM | 5951 | N | PHE | B | 1806 | −4.889 | −48.840 | 84.119 | 1.00 | 210.37 | N |
| ATOM | 5952 | CA | PHE | B | 1806 | −4.560 | −47.543 | 84.709 | 1.00 | 210.10 | C |
| ATOM | 5953 | C | PHE | B | 1806 | −3.582 | −46.781 | 83.795 | 1.00 | 209.86 | C |
| ATOM | 5954 | O | PHE | B | 1806 | −2.462 | −47.246 | 83.550 | 1.00 | 209.98 | O |
| ATOM | 5955 | CB | PHE | B | 1806 | −3.952 | −47.764 | 86.101 | 1.00 | 210.09 | C |
| ATOM | 5956 | CG | PHE | B | 1806 | −4.041 | −46.571 | 87.005 | 1.00 | 210.14 | C |
| ATOM | 5957 | CD1 | PHE | B | 1806 | −2.886 | −45.949 | 87.468 | 1.00 | 210.28 | C |
| ATOM | 5958 | CD2 | PHE | B | 1806 | −5.281 | −46.071 | 87.402 | 1.00 | 210.25 | C |
| ATOM | 5959 | CE1 | PHE | B | 1806 | −2.961 | −44.840 | 88.312 | 1.00 | 210.49 | C |
| ATOM | 5960 | CE2 | PHE | B | 1806 | −5.370 | −44.962 | 88.243 | 1.00 | 210.38 | C |
| ATOM | 5961 | CZ | PHE | B | 1806 | −4.208 | −44.344 | 88.700 | 1.00 | 210.41 | C |
| ATOM | 5962 | N | VAL | B | 1807 | −4.011 | −45.626 | 83.279 | 1.00 | 209.36 | N |
| ATOM | 5963 | CA | VAL | B | 1807 | −3.184 | −44.848 | 82.346 | 1.00 | 208.86 | C |
| ATOM | 5964 | C | VAL | B | 1807 | −2.764 | −43.500 | 82.933 | 1.00 | 208.67 | C |
| ATOM | 5965 | O | VAL | B | 1807 | −3.515 | −42.518 | 82.882 | 1.00 | 208.50 | O |
| ATOM | 5966 | CB | VAL | B | 1807 | −3.876 | −44.644 | 80.980 | 1.00 | 208.79 | C |
| ATOM | 5967 | CG1 | VAL | B | 1807 | −2.934 | −43.967 | 79.999 | 1.00 | 208.75 | C |
| ATOM | 5968 | CG2 | VAL | B | 1807 | −4.357 | −45.971 | 80.422 | 1.00 | 208.69 | C |
| ATOM | 5969 | N | LYS | B | 1808 | −1.549 | −43.476 | 83.482 | 1.00 | 208.51 | N |
| ATOM | 5970 | CA | LYS | B | 1808 | −0.981 | −42.296 | 84.139 | 1.00 | 208.29 | C |
| ATOM | 5971 | C | LYS | B | 1808 | −0.643 | −41.197 | 83.134 | 1.00 | 208.28 | C |
| ATOM | 5972 | O | LYS | B | 1808 | −0.267 | −41.496 | 82.001 | 1.00 | 208.29 | O |
| ATOM | 5973 | CB | LYS | B | 1808 | 0.266 | −42.681 | 84.948 | 1.00 | 208.30 | C |
| ATOM | 5974 | CG | LYS | B | 1808 | −0.026 | −43.299 | 86.319 | 1.00 | 208.10 | C |
| ATOM | 5975 | CD | LYS | B | 1808 | 1.254 | −43.516 | 87.115 | 1.00 | 207.97 | C |
| ATOM | 5976 | CE | LYS | B | 1808 | 0.964 | −44.013 | 88.516 | 1.00 | 207.27 | C |
| ATOM | 5977 | NZ | LYS | B | 1808 | 2.227 | −44.200 | 89.276 | 1.00 | 207.11 | N |
| ATOM | 5978 | N | PRO | B | 1809 | −0.781 | −39.919 | 83.543 | 1.00 | 208.25 | N |
| ATOM | 5979 | CA | PRO | B | 1809 | −0.525 | −38.788 | 82.652 | 1.00 | 208.38 | C |
| ATOM | 5980 | C | PRO | B | 1809 | 0.816 | −38.875 | 81.917 | 1.00 | 208.60 | C |
| ATOM | 5981 | O | PRO | B | 1809 | 1.810 | −39.334 | 82.484 | 1.00 | 208.58 | O |
| ATOM | 5982 | CB | PRO | B | 1809 | −0.554 | −37.593 | 83.603 | 1.00 | 208.30 | C |
| ATOM | 5983 | CG | PRO | B | 1809 | −1.496 | −38.016 | 84.667 | 1.00 | 208.14 | C |
| ATOM | 5984 | CD | PRO | B | 1809 | −1.201 | −39.461 | 84.880 | 1.00 | 208.16 | C |
| ATOM | 5985 | N | ASN | B | 1810 | 0.811 | −38.443 | 80.656 | 1.00 | 208.87 | N |
| ATOM | 5986 | CA | ASN | B | 1810 | 1.960 | −38.533 | 79.738 | 1.00 | 209.21 | C |
| ATOM | 5987 | C | ASN | B | 1810 | 2.474 | −39.939 | 79.377 | 1.00 | 208.54 | C |
| ATOM | 5988 | O | ASN | B | 1810 | 3.232 | −40.084 | 78.414 | 1.00 | 208.62 | O |
| ATOM | 5989 | CB | ASN | B | 1810 | 3.110 | −37.601 | 80.158 | 1.00 | 209.77 | C |
| ATOM | 5990 | CG | ASN | B | 1810 | 3.029 | −36.236 | 79.486 | 1.00 | 212.63 | C |
| ATOM | 5991 | OD1 | ASN | B | 1810 | 2.383 | −36.078 | 78.441 | 1.00 | 213.00 | O |
| ATOM | 5992 | ND2 | ASN | B | 1810 | 3.708 | −35.242 | 80.076 | 1.00 | 217.71 | N |
| ATOM | 5993 | N | GLU | B | 1811 | 2.064 | −40.966 | 80.124 | 1.00 | 207.70 | N |
| ATOM | 5994 | CA | GLU | B | 1811 | 2.414 | −42.343 | 79.756 | 1.00 | 206.72 | C |
| ATOM | 5995 | C | GLU | B | 1811 | 1.510 | −42.812 | 78.616 | 1.00 | 206.10 | C |
| ATOM | 5996 | O | GLU | B | 1811 | 0.451 | −42.226 | 78.367 | 1.00 | 205.91 | O |
| ATOM | 5997 | CB | GLU | B | 1811 | 2.406 | −43.303 | 80.971 | 1.00 | 206.81 | C |
| ATOM | 5998 | CG | GLU | B | 1811 | 1.136 | −44.156 | 81.190 | 1.00 | 206.67 | C |
| ATOM | 5999 | CD | GLU | B | 1811 | 1.351 | −45.350 | 82.133 | 1.00 | 206.49 | C |
| ATOM | 6000 | OE1 | GLU | B | 1811 | 2.355 | −45.369 | 82.876 | 1.00 | 206.29 | O |
| ATOM | 6001 | OE2 | GLU | B | 1811 | 0.509 | −46.275 | 82.131 | 1.00 | 205.81 | O |
| ATOM | 6002 | N | THR | B | 1812 | 1.949 | −43.852 | 77.915 | 1.00 | 205.33 | N |
| ATOM | 6003 | CA | THR | B | 1812 | 1.220 | −44.370 | 76.766 | 1.00 | 204.60 | C |
| ATOM | 6004 | C | THR | B | 1812 | 0.799 | −45.824 | 77.010 | 1.00 | 204.02 | C |
| ATOM | 6005 | O | THR | B | 1812 | 1.640 | −46.724 | 77.074 | 1.00 | 203.91 | O |
| ATOM | 6006 | CB | THR | B | 1812 | 2.061 | −44.226 | 75.463 | 1.00 | 204.69 | C |
| ATOM | 6007 | OG1 | THR | B | 1812 | 2.546 | −42.881 | 75.348 | 1.00 | 204.57 | O |
| ATOM | 6008 | CG2 | THR | B | 1812 | 1.242 | −44.568 | 74.223 | 1.00 | 204.69 | C |
| ATOM | 6009 | N | LYS | B | 1813 | −0.503 | −46.038 | 77.178 | 1.00 | 203.35 | N |
| ATOM | 6010 | CA | LYS | B | 1813 | −1.047 | −47.393 | 77.275 | 1.00 | 202.80 | C |
| ATOM | 6011 | C | LYS | B | 1813 | −1.778 | −47.775 | 75.999 | 1.00 | 202.33 | C |
| ATOM | 6012 | O | LYS | B | 1813 | −2.846 | −47.249 | 75.679 | 1.00 | 202.16 | O |
| ATOM | 6013 | CB | LYS | B | 1813 | −1.936 | −47.590 | 78.511 | 1.00 | 202.86 | C |
| ATOM | 6014 | CG | LYS | B | 1813 | −1.171 | −48.023 | 79.760 | 1.00 | 202.79 | C |
| ATOM | 6015 | CD | LYS | B | 1813 | −1.980 | −48.989 | 80.613 | 1.00 | 202.44 | C |
| ATOM | 6016 | CE | LYS | B | 1813 | −1.094 | −49.691 | 81.637 | 1.00 | 202.20 | C |
| ATOM | 6017 | NZ | LYS | B | 1813 | −1.797 | −50.826 | 82.305 | 1.00 | 201.89 | N |
| ATOM | 6018 | N | THR | B | 1814 | −1.174 | −48.705 | 75.278 | 1.00 | 201.81 | N |
| ATOM | 6019 | CA | THR | B | 1814 | −1.638 | −49.096 | 73.968 | 1.00 | 201.31 | C |
| ATOM | 6020 | C | THR | B | 1814 | −2.324 | −50.455 | 74.035 | 1.00 | 201.12 | C |
| ATOM | 6021 | O | THR | B | 1814 | −1.689 | −51.441 | 74.433 | 1.00 | 201.09 | O |
| ATOM | 6022 | CB | THR | B | 1814 | −0.455 | −49.130 | 72.972 | 1.00 | 201.29 | C |
| ATOM | 6023 | OG1 | THR | B | 1814 | −0.798 | −49.936 | 71.840 | 1.00 | 201.11 | O |
| ATOM | 6024 | CG2 | THR | B | 1814 | 0.825 | −49.679 | 73.639 | 1.00 | 201.01 | C |
| ATOM | 6025 | N | TYR | B | 1815 | −3.613 | −50.508 | 73.670 | 1.00 | 200.80 | N |
| ATOM | 6026 | CA | TYR | B | 1815 | −4.318 | −51.807 | 73.581 | 1.00 | 200.44 | C |
| ATOM | 6027 | C | TYR | B | 1815 | −5.158 | −52.094 | 72.325 | 1.00 | 199.62 | C |
| ATOM | 6028 | O | TYR | B | 1815 | −5.761 | −51.198 | 71.740 | 1.00 | 199.26 | O |
| ATOM | 6029 | CB | TYR | B | 1815 | −5.084 | −52.155 | 74.865 | 1.00 | 200.96 | C |
| ATOM | 6030 | CG | TYR | B | 1815 | −4.726 | −53.544 | 75.378 | 1.00 | 201.91 | C |
| ATOM | 6031 | CD1 | TYR | B | 1815 | −5.504 | −54.660 | 75.047 | 1.00 | 202.48 | C |
| ATOM | 6032 | CD2 | TYR | B | 1815 | −3.579 | −53.747 | 76.162 | 1.00 | 202.41 | C |
| ATOM | 6033 | CE1 | TYR | B | 1815 | −5.165 | −55.940 | 75.497 | 1.00 | 202.38 | C |
| ATOM | 6034 | CE2 | TYR | B | 1815 | −3.232 | −55.019 | 76.617 | 1.00 | 202.43 | C |
| ATOM | 6035 | CZ | TYR | B | 1815 | −4.030 | −56.109 | 76.281 | 1.00 | 202.28 | C |
| ATOM | 6036 | OH | TYR | B | 1815 | −3.696 | −57.368 | 76.732 | 1.00 | 202.30 | O |
| ATOM | 6037 | N | PHE | B | 1816 | −5.182 | −53.380 | 71.959 | 1.00 | 198.87 | N |
| ATOM | 6038 | CA | PHE | B | 1816 | −5.597 | −53.865 | 70.644 | 1.00 | 198.17 | C |
| ATOM | 6039 | C | PHE | B | 1816 | −5.931 | −55.366 | 70.666 | 1.00 | 197.98 | C |
| ATOM | 6040 | O | PHE | B | 1816 | −5.219 | −56.163 | 71.273 | 1.00 | 197.83 | O |
| ATOM | 6041 | CB | PHE | B | 1816 | −4.519 | −53.505 | 69.612 | 1.00 | 198.02 | C |
| ATOM | 6042 | CG | PHE | B | 1816 | −4.069 | −54.640 | 68.754 | 1.00 | 197.37 | C |
| ATOM | 6043 | CD1 | PHE | B | 1816 | −2.862 | −55.265 | 69.009 | 1.00 | 197.16 | C |
| ATOM | 6044 | CD2 | PHE | B | 1816 | −4.824 | −55.056 | 67.670 | 1.00 | 197.01 | C |
| ATOM | 6045 | CE1 | PHE | B | 1816 | −2.424 | −56.305 | 68.216 | 1.00 | 197.28 | C |
| ATOM | 6046 | CE2 | PHE | B | 1816 | −4.398 | −56.095 | 66.874 | 1.00 | 197.04 | C |
| ATOM | 6047 | CZ | PHE | B | 1816 | −3.193 | −56.721 | 67.145 | 1.00 | 197.46 | C |
| ATOM | 6048 | N | TRP | B | 1817 | −7.016 | −55.726 | 69.981 | 1.00 | 197.98 | N |
| ATOM | 6049 | CA | TRP | B | 1817 | −7.739 | −56.997 | 70.190 | 1.00 | 198.05 | C |
| ATOM | 6050 | C | TRP | B | 1817 | −8.625 | −57.379 | 69.002 | 1.00 | 197.76 | C |
| ATOM | 6051 | O | TRP | B | 1817 | −9.194 | −56.507 | 68.338 | 1.00 | 197.77 | O |
| ATOM | 6052 | CB | TRP | B | 1817 | −8.641 | −56.882 | 71.430 | 1.00 | 198.32 | C |
| ATOM | 6053 | CG | TRP | B | 1817 | −9.231 | −55.480 | 71.620 | 1.00 | 198.83 | C |
| ATOM | 6054 | CD1 | TRP | B | 1817 | −8.625 | −54.413 | 72.230 | 1.00 | 199.31 | C |
| ATOM | 6055 | CD2 | TRP | B | 1817 | −10.517 | −55.007 | 71.191 | 1.00 | 198.74 | C |
| ATOM | 6056 | NE1 | TRP | B | 1817 | −9.447 | −53.312 | 72.208 | 1.00 | 198.99 | N |
| ATOM | 6057 | CE2 | TRP | B | 1817 | −10.615 | −53.644 | 71.579 | 1.00 | 198.36 | C |
| ATOM | 6058 | CE3 | TRP | B | 1817 | −11.592 | −55.595 | 70.521 | 1.00 | 198.82 | C |
| ATOM | 6059 | CZ2 | TRP | B | 1817 | −11.736 | −52.865 | 71.320 | 1.00 | 197.90 | C |
| ATOM | 6060 | CZ3 | TRP | B | 1817 | −12.713 | −54.814 | 70.264 | 1.00 | 199.16 | C |
| ATOM | 6061 | CH2 | TRP | B | 1817 | −12.772 | −53.459 | 70.664 | 1.00 | 198.67 | C |
| ATOM | 6062 | N | LYS | B | 1818 | −8.768 | −58.677 | 68.746 | 1.00 | 197.35 | N |
| ATOM | 6063 | CA | LYS | B | 1818 | −9.698 | −59.126 | 67.719 | 1.00 | 196.94 | C |
| ATOM | 6064 | C | LYS | B | 1818 | −11.089 | −58.756 | 68.184 | 1.00 | 196.73 | C |
| ATOM | 6065 | O | LYS | B | 1818 | −11.418 | −58.938 | 69.352 | 1.00 | 196.71 | O |
| ATOM | 6066 | CB | LYS | B | 1818 | −9.601 | −60.636 | 67.505 | 1.00 | 196.90 | C |
| ATOM | 6067 | CG | LYS | B | 1818 | −10.314 | −61.155 | 66.249 | 1.00 | 196.89 | C |
| ATOM | 6068 | CD | LYS | B | 1818 | −9.396 | −61.160 | 65.016 | 1.00 | 197.01 | C |
| ATOM | 6069 | CE | LYS | B | 1818 | −10.072 | −61.776 | 63.776 | 1.00 | 196.86 | C |
| ATOM | 6070 | NZ | LYS | B | 1818 | −10.063 | −63.279 | 63.737 | 1.00 | 196.43 | N |
| ATOM | 6071 | N | VAL | B | 1819 | −11.888 | −58.195 | 67.285 | 1.00 | 196.58 | N |
| ATOM | 6072 | CA | VAL | B | 1819 | −13.286 | −57.910 | 67.595 | 1.00 | 196.52 | C |
| ATOM | 6073 | C | VAL | B | 1819 | −14.172 | −59.036 | 67.091 | 1.00 | 196.51 | C |
| ATOM | 6074 | O | VAL | B | 1819 | −14.694 | −58.993 | 65.980 | 1.00 | 196.51 | O |
| ATOM | 6075 | CB | VAL | B | 1819 | −13.771 | −56.529 | 67.069 | 1.00 | 196.53 | C |
| ATOM | 6076 | CG1 | VAL | B | 1819 | −13.385 | −56.304 | 65.611 | 1.00 | 196.27 | C |
| ATOM | 6077 | CG2 | VAL | B | 1819 | −15.279 | −56.383 | 67.269 | 1.00 | 196.58 | C |
| ATOM | 6078 | N | GLN | B | 1820 | −14.328 | −60.049 | 67.931 | 1.00 | 196.55 | N |
| ATOM | 6079 | CA | GLN | B | 1820 | −15.114 | −61.235 | 67.608 | 1.00 | 196.52 | C |
| ATOM | 6080 | C | GLN | B | 1820 | −16.580 | −60.888 | 67.282 | 1.00 | 196.35 | C |
| ATOM | 6081 | O | GLN | B | 1820 | −17.014 | −59.740 | 67.453 | 1.00 | 196.35 | O |
| ATOM | 6082 | CB | GLN | B | 1820 | −15.003 | −62.237 | 68.774 | 1.00 | 196.60 | C |
| ATOM | 6083 | CG | GLN | B | 1820 | −15.792 | −63.537 | 68.628 | 1.00 | 196.91 | C |
| ATOM | 6084 | CD | GLN | B | 1820 | −15.332 | −64.398 | 67.462 | 1.00 | 197.16 | C |
| ATOM | 6085 | OE1 | GLN | B | 1820 | −14.139 | −64.637 | 67.279 | 1.00 | 197.10 | O |
| ATOM | 6086 | NE2 | GLN | B | 1820 | −16.288 | −64.878 | 66.675 | 1.00 | 197.48 | N |
| ATOM | 6087 | N | HIS | B | 1821 | −17.314 | −61.888 | 66.793 | 1.00 | 196.09 | N |
| ATOM | 6088 | CA | HIS | B | 1821 | −18.757 | −61.826 | 66.577 | 1.00 | 196.11 | C |
| ATOM | 6089 | C | HIS | B | 1821 | −19.562 | −61.590 | 67.894 | 1.00 | 195.57 | C |
| ATOM | 6090 | O | HIS | B | 1821 | −20.764 | −61.261 | 67.874 | 1.00 | 195.50 | O |
| ATOM | 6091 | CB | HIS | B | 1821 | −19.188 | −63.096 | 65.843 | 1.00 | 196.53 | C |
| ATOM | 6092 | CG | HIS | B | 1821 | −20.651 | −63.162 | 65.552 | 1.00 | 198.60 | C |
| ATOM | 6093 | ND1 | HIS | B | 1821 | −21.485 | −64.087 | 66.145 | 1.00 | 200.17 | N |
| ATOM | 6094 | CD2 | HIS | B | 1821 | −21.435 | −62.406 | 64.746 | 1.00 | 200.21 | C |
| ATOM | 6095 | CE1 | HIS | B | 1821 | −22.721 | −63.902 | 65.714 | 1.00 | 200.86 | C |
| ATOM | 6096 | NE2 | HIS | B | 1821 | −22.718 | −62.888 | 64.864 | 1.00 | 201.27 | N |
| ATOM | 6097 | N | HIS | B | 1822 | −18.877 | −61.776 | 69.025 | 1.00 | 194.84 | N |
| ATOM | 6098 | CA | HIS | B | 1822 | −19.244 | −61.215 | 70.335 | 1.00 | 193.93 | C |
| ATOM | 6099 | C | HIS | B | 1822 | −19.878 | −59.814 | 70.169 | 1.00 | 193.06 | C |
| ATOM | 6100 | O | HIS | B | 1822 | −21.079 | −59.657 | 70.397 | 1.00 | 192.63 | O |
| ATOM | 6101 | CB | HIS | B | 1822 | −17.962 | −61.155 | 71.194 | 1.00 | 194.16 | C |
| ATOM | 6102 | CG | HIS | B | 1822 | −18.178 | −61.306 | 72.671 | 1.00 | 194.78 | C |
| ATOM | 6103 | ND1 | HIS | B | 1822 | −19.332 | −61.826 | 73.218 | 1.00 | 195.61 | N |
| ATOM | 6104 | CD2 | HIS | B | 1822 | −17.355 | −61.041 | 73.715 | 1.00 | 195.28 | C |
| ATOM | 6105 | CE1 | HIS | B | 1822 | −19.221 | −61.849 | 74.537 | 1.00 | 195.80 | C |
| ATOM | 6106 | NE2 | HIS | B | 1822 | −18.031 | −61.376 | 74.864 | 1.00 | 195.77 | N |
| ATOM | 6107 | N | MET | B | 1823 | −19.072 | −58.836 | 69.721 | 1.00 | 192.13 | N |
| ATOM | 6108 | CA | MET | B | 1823 | −19.487 | −57.429 | 69.508 | 1.00 | 191.43 | C |
| ATOM | 6109 | C | MET | B | 1823 | −20.305 | −57.147 | 68.222 | 1.00 | 190.57 | C |
| ATOM | 6110 | O | MET | B | 1823 | −20.755 | −56.028 | 67.993 | 1.00 | 190.10 | O |
| ATOM | 6111 | CB | MET | B | 1823 | −18.258 | −56.483 | 69.537 | 1.00 | 191.49 | C |
| ATOM | 6112 | CG | MET | B | 1823 | −17.713 | −55.955 | 70.949 | 1.00 | 192.23 | C |
| ATOM | 6113 | SD | MET | B | 1823 | −18.126 | −54.324 | 71.774 | 1.00 | 191.97 | S |
| ATOM | 6114 | CE | MET | B | 1823 | −16.525 | −53.597 | 72.121 | 1.00 | 190.96 | C |
| ATOM | 6115 | N | ALA | B | 1824 | −20.504 | −58.148 | 67.383 | 1.00 | 190.08 | N |
| ATOM | 6116 | CA | ALA | B | 1824 | −21.144 | −57.913 | 66.091 | 1.00 | 189.63 | C |
| ATOM | 6117 | C | ALA | B | 1824 | −22.662 | −57.920 | 66.163 | 1.00 | 189.26 | C |
| ATOM | 6118 | O | ALA | B | 1824 | −23.240 | −58.674 | 66.953 | 1.00 | 189.36 | O |
| ATOM | 6119 | CB | ALA | B | 1824 | −20.668 | −58.938 | 65.069 | 1.00 | 189.81 | C |
| ATOM | 6120 | N | PRO | B | 1825 | −23.310 | −57.062 | 65.353 | 1.00 | 188.73 | N |
| ATOM | 6121 | CA | PRO | B | 1825 | −24.732 | −57.130 | 65.012 | 1.00 | 188.61 | C |
| ATOM | 6122 | C | PRO | B | 1825 | −25.048 | −58.314 | 64.097 | 1.00 | 188.72 | C |
| ATOM | 6123 | O | PRO | B | 1825 | −24.141 | −58.851 | 63.470 | 1.00 | 188.66 | O |
| ATOM | 6124 | CB | PRO | B | 1825 | −24.974 | −55.816 | 64.280 | 1.00 | 188.37 | C |
| ATOM | 6125 | CG | PRO | B | 1825 | −23.673 | −55.455 | 63.747 | 1.00 | 188.27 | C |
| ATOM | 6126 | CD | PRO | B | 1825 | −22.673 | −55.885 | 64.752 | 1.00 | 188.44 | C |
| ATOM | 6127 | N | THR | B | 1826 | −26.325 | −58.698 | 64.017 | 1.00 | 189.08 | N |
| ATOM | 6128 | CA | THR | B | 1826 | −26.747 | −59.966 | 63.392 | 1.00 | 189.53 | C |
| ATOM | 6129 | C | THR | B | 1826 | −27.646 | −59.815 | 62.159 | 1.00 | 190.06 | C |
| ATOM | 6130 | O | THR | B | 1826 | −28.049 | −58.709 | 61.812 | 1.00 | 189.92 | O |
| ATOM | 6131 | CB | THR | B | 1826 | −27.500 | −60.868 | 64.403 | 1.00 | 189.46 | C |
| ATOM | 6132 | OG1 | THR | B | 1826 | −28.646 | −60.176 | 64.907 | 1.00 | 189.10 | O |
| ATOM | 6133 | CG2 | THR | B | 1826 | −26.603 | −61.283 | 65.562 | 1.00 | 189.48 | C |
| ATOM | 6134 | N | LYS | B | 1827 | −27.967 | −60.947 | 61.523 | 1.00 | 191.02 | N |
| ATOM | 6135 | CA | LYS | B | 1827 | −28.852 | −61.005 | 60.335 | 1.00 | 192.08 | C |
| ATOM | 6136 | C | LYS | B | 1827 | −30.244 | −60.451 | 60.600 | 1.00 | 192.81 | C |
| ATOM | 6137 | O | LYS | B | 1827 | −30.920 | −59.977 | 59.679 | 1.00 | 192.88 | O |
| ATOM | 6138 | CB | LYS | B | 1827 | −28.954 | −62.441 | 59.746 | 1.00 | 192.13 | C |
| ATOM | 6139 | CG | LYS | B | 1827 | −29.965 | −63.457 | 60.403 | 1.00 | 191.92 | C |
| ATOM | 6140 | CD | LYS | B | 1827 | −31.413 | −63.388 | 59.845 | 1.00 | 191.21 | C |
| ATOM | 6141 | CE | LYS | B | 1827 | −31.549 | −63.870 | 58.394 | 1.00 | 190.86 | C |
| ATOM | 6142 | NZ | LYS | B | 1827 | −31.738 | −65.336 | 58.269 | 1.00 | 190.46 | N |
| ATOM | 6143 | N | ASP | B | 1828 | −30.667 | −60.563 | 61.859 | 1.00 | 193.73 | N |
| ATOM | 6144 | CA | ASP | B | 1828 | −31.933 | −60.024 | 62.321 | 1.00 | 194.60 | C |
| ATOM | 6145 | C | ASP | B | 1828 | −31.785 | −58.500 | 62.475 | 1.00 | 194.84 | C |
| ATOM | 6146 | O | ASP | B | 1828 | −32.749 | −57.752 | 62.263 | 1.00 | 195.06 | O |
| ATOM | 6147 | CB | ASP | B | 1828 | −32.353 | −60.676 | 63.660 | 1.00 | 194.87 | C |
| ATOM | 6148 | CG | ASP | B | 1828 | −32.694 | −62.191 | 63.538 | 1.00 | 196.15 | C |
| ATOM | 6149 | OD1 | ASP | B | 1828 | −33.840 | −62.571 | 63.889 | 1.00 | 197.40 | O |
| ATOM | 6150 | OD2 | ASP | B | 1828 | −31.825 | −63.007 | 63.130 | 1.00 | 197.06 | O |
| ATOM | 6151 | N | GLU | B | 1829 | −30.565 | −58.050 | 62.790 | 1.00 | 195.01 | N |
| ATOM | 6152 | CA | GLU | B | 1829 | −30.318 | −56.668 | 63.236 | 1.00 | 195.36 | C |
| ATOM | 6153 | C | GLU | B | 1829 | −30.159 | −55.596 | 62.137 | 1.00 | 195.11 | C |
| ATOM | 6154 | O | GLU | B | 1829 | −30.805 | −55.681 | 61.089 | 1.00 | 194.92 | O |
| ATOM | 6155 | CB | GLU | B | 1829 | −29.169 | −56.628 | 64.267 | 1.00 | 195.38 | C |
| ATOM | 6156 | CG | GLU | B | 1829 | −29.632 | −56.875 | 65.724 | 1.00 | 196.05 | C |
| ATOM | 6157 | CD | GLU | B | 1829 | −28.491 | −56.913 | 66.745 | 1.00 | 196.36 | C |
| ATOM | 6158 | OE1 | GLU | B | 1829 | −28.507 | −56.090 | 67.689 | 1.00 | 197.43 | O |
| ATOM | 6159 | OE2 | GLU | B | 1829 | −27.583 | −57.769 | 66.614 | 1.00 | 198.14 | O |
| ATOM | 6160 | N | PHE | B | 1830 | −29.345 | −54.571 | 62.422 | 1.00 | 195.19 | N |
| ATOM | 6161 | CA | PHE | B | 1830 | −29.056 | −53.463 | 61.493 | 1.00 | 195.28 | C |
| ATOM | 6162 | C | PHE | B | 1830 | −27.667 | −53.547 | 60.896 | 1.00 | 195.66 | C |
| ATOM | 6163 | O | PHE | B | 1830 | −26.855 | −54.363 | 61.319 | 1.00 | 196.05 | O |
| ATOM | 6164 | CB | PHE | B | 1830 | −29.215 | −52.104 | 62.171 | 1.00 | 194.83 | C |
| ATOM | 6165 | CG | PHE | B | 1830 | −30.611 | −51.584 | 62.139 | 1.00 | 194.63 | C |
| ATOM | 6166 | CD1 | PHE | B | 1830 | −31.617 | −52.283 | 61.466 | 1.00 | 194.64 | C |
| ATOM | 6167 | CD2 | PHE | B | 1830 | −30.930 | −50.391 | 62.759 | 1.00 | 194.41 | C |
| ATOM | 6168 | CE1 | PHE | B | 1830 | −32.921 | −51.807 | 61.424 | 1.00 | 194.13 | C |
| ATOM | 6169 | CE2 | PHE | B | 1830 | −32.236 | −49.905 | 62.722 | 1.00 | 194.49 | C |
| ATOM | 6170 | CZ | PHE | B | 1830 | −33.231 | −50.615 | 62.048 | 1.00 | 194.26 | C |
| ATOM | 6171 | N | ASP | B | 1831 | −27.385 | −52.687 | 59.923 | 1.00 | 195.93 | N |
| ATOM | 6172 | CA | ASP | B | 1831 | −26.082 | −52.695 | 59.262 | 1.00 | 196.23 | C |
| ATOM | 6173 | C | ASP | B | 1831 | −24.944 | −52.403 | 60.230 | 1.00 | 195.98 | C |
| ATOM | 6174 | O | ASP | B | 1831 | −23.789 | −52.684 | 59.915 | 1.00 | 195.98 | O |
| ATOM | 6175 | CB | ASP | B | 1831 | −26.053 | −51.697 | 58.109 | 1.00 | 196.57 | C |
| ATOM | 6176 | CG | ASP | B | 1831 | −27.137 | −51.963 | 57.084 | 1.00 | 197.68 | C |
| ATOM | 6177 | OD1 | ASP | B | 1831 | −28.118 | −51.170 | 57.050 | 1.00 | 198.39 | O |
| ATOM | 6178 | OD2 | ASP | B | 1831 | −27.010 | −52.971 | 56.335 | 1.00 | 198.64 | O |
| ATOM | 6179 | N | CYS | B | 1832 | −25.277 | −51.845 | 61.398 | 1.00 | 195.83 | N |
| ATOM | 6180 | CA | CYS | B | 1832 | −24.295 | −51.596 | 62.452 | 1.00 | 195.42 | C |
| ATOM | 6181 | C | CYS | B | 1832 | −24.913 | −51.409 | 63.846 | 1.00 | 195.00 | C |
| ATOM | 6182 | O | CYS | B | 1832 | −25.928 | −50.725 | 63.987 | 1.00 | 195.13 | O |
| ATOM | 6183 | CB | CYS | B | 1832 | −23.436 | −50.386 | 62.090 | 1.00 | 195.49 | C |
| ATOM | 6184 | SG | CYS | B | 1832 | −21.662 | −50.676 | 62.358 | 1.00 | 196.71 | S |
| ATOM | 6185 | N | LYS | B | 1833 | −24.293 | −52.023 | 64.859 | 1.00 | 194.40 | N |
| ATOM | 6186 | CA | LYS | B | 1833 | −24.697 | −51.879 | 66.266 | 1.00 | 193.76 | C |
| ATOM | 6187 | C | LYS | B | 1833 | −23.869 | −50.765 | 66.939 | 1.00 | 193.43 | C |
| ATOM | 6188 | O | LYS | B | 1833 | −22.865 | −50.308 | 66.384 | 1.00 | 193.24 | O |
| ATOM | 6189 | CB | LYS | B | 1833 | −24.552 | −53.223 | 67.008 | 1.00 | 193.73 | C |
| ATOM | 6190 | CG | LYS | B | 1833 | −25.675 | −53.560 | 68.016 | 1.00 | 193.71 | C |
| ATOM | 6191 | CD | LYS | B | 1833 | −25.409 | −53.014 | 69.438 | 1.00 | 194.43 | C |
| ATOM | 6192 | CE | LYS | B | 1833 | −26.695 | −52.808 | 70.278 | 1.00 | 194.07 | C |
| ATOM | 6193 | NZ | LYS | B | 1833 | −26.462 | −52.057 | 71.563 | 1.00 | 193.00 | N |
| ATOM | 6194 | N | ALA | B | 1834 | −24.295 | −50.327 | 68.122 | 1.00 | 193.21 | N |
| ATOM | 6195 | CA | ALA | B | 1834 | −23.642 | −49.214 | 68.827 | 1.00 | 193.07 | C |
| ATOM | 6196 | C | ALA | B | 1834 | −23.219 | −49.523 | 70.265 | 1.00 | 192.86 | C |
| ATOM | 6197 | O | ALA | B | 1834 | −23.999 | −50.059 | 71.060 | 1.00 | 192.75 | O |
| ATOM | 6198 | CB | ALA | B | 1834 | −24.529 | −47.992 | 68.809 | 1.00 | 193.35 | C |
| ATOM | 6199 | N | TRP | B | 1835 | −21.984 | −49.143 | 70.587 | 1.00 | 192.52 | N |
| ATOM | 6200 | CA | TRP | B | 1835 | −21.340 | −49.503 | 71.843 | 1.00 | 192.27 | C |
| ATOM | 6201 | C | TRP | B | 1835 | −20.635 | −48.318 | 72.452 | 1.00 | 192.41 | C |
| ATOM | 6202 | O | TRP | B | 1835 | −20.220 | −47.408 | 71.744 | 1.00 | 192.55 | O |
| ATOM | 6203 | CB | TRP | B | 1835 | −20.313 | −50.604 | 71.598 | 1.00 | 191.96 | C |
| ATOM | 6204 | CG | TRP | B | 1835 | −20.936 | −51.871 | 71.149 | 1.00 | 191.52 | C |
| ATOM | 6205 | CD1 | TRP | B | 1835 | −21.290 | −52.204 | 69.878 | 1.00 | 191.66 | C |
| ATOM | 6206 | CD2 | TRP | B | 1835 | −21.306 | −52.973 | 71.968 | 1.00 | 190.64 | C |
| ATOM | 6207 | NE1 | TRP | B | 1835 | −21.856 | −53.455 | 69.852 | 1.00 | 191.16 | N |
| ATOM | 6208 | CE2 | TRP | B | 1835 | −21.876 | −53.947 | 71.125 | 1.00 | 190.40 | C |
| ATOM | 6209 | CE3 | TRP | B | 1835 | −21.210 | −53.235 | 73.333 | 1.00 | 191.02 | C |
| ATOM | 6210 | CZ2 | TRP | B | 1835 | −22.344 | −55.154 | 71.599 | 1.00 | 190.99 | C |
| ATOM | 6211 | CZ3 | TRP | B | 1835 | −21.675 | −54.436 | 73.804 | 1.00 | 191.53 | C |
| ATOM | 6212 | CH2 | TRP | B | 1835 | −22.236 | −55.382 | 72.940 | 1.00 | 191.59 | C |
| ATOM | 6213 | N | ALA | B | 1836 | −20.469 | −48.347 | 73.765 | 1.00 | 192.61 | N |
| ATOM | 6214 | CA | ALA | B | 1836 | −19.832 | −47.248 | 74.464 | 1.00 | 192.99 | C |
| ATOM | 6215 | C | ALA | B | 1836 | −18.403 | −47.568 | 74.879 | 1.00 | 193.29 | C |
| ATOM | 6216 | O | ALA | B | 1836 | −18.086 | −48.713 | 75.193 | 1.00 | 193.29 | O |
| ATOM | 6217 | CB | ALA | B | 1836 | −20.652 | −46.865 | 75.672 | 1.00 | 193.02 | C |
| ATOM | 6218 | N | TYR | B | 1837 | −17.547 | −46.549 | 74.845 | 1.00 | 193.72 | N |
| ATOM | 6219 | CA | TYR | B | 1837 | −16.269 | −46.577 | 75.550 | 1.00 | 194.27 | C |
| ATOM | 6220 | C | TYR | B | 1837 | −16.199 | −45.390 | 76.485 | 1.00 | 194.93 | C |
| ATOM | 6221 | O | TYR | B | 1837 | −16.749 | −44.334 | 76.170 | 1.00 | 195.07 | O |
| ATOM | 6222 | CB | TYR | B | 1837 | −15.060 | −46.591 | 74.600 | 1.00 | 194.05 | C |
| ATOM | 6223 | CG | TYR | B | 1837 | −14.774 | −45.328 | 73.784 | 1.00 | 193.92 | C |
| ATOM | 6224 | CD1 | TYR | B | 1837 | −14.128 | −44.222 | 74.345 | 1.00 | 193.66 | C |
| ATOM | 6225 | CD2 | TYR | B | 1837 | −15.087 | −45.275 | 72.426 | 1.00 | 193.98 | C |
| ATOM | 6226 | CE1 | TYR | B | 1837 | −13.849 | −43.082 | 73.577 | 1.00 | 193.37 | C |
| ATOM | 6227 | CE2 | TYR | B | 1837 | −14.808 | −44.146 | 71.658 | 1.00 | 193.52 | C |
| ATOM | 6228 | CZ | TYR | B | 1837 | −14.193 | −43.059 | 72.233 | 1.00 | 193.41 | C |
| ATOM | 6229 | OH | TYR | B | 1837 | −13.939 | −41.959 | 71.450 | 1.00 | 193.43 | O |
| ATOM | 6230 | N | PHE | B | 1838 | −15.537 | −45.571 | 77.631 | 1.00 | 195.79 | N |
| ATOM | 6231 | CA | PHE | B | 1838 | −15.315 | −44.485 | 78.614 | 1.00 | 196.54 | C |
| ATOM | 6232 | C | PHE | B | 1838 | −14.152 | −44.763 | 79.570 | 1.00 | 196.79 | C |
| ATOM | 6233 | O | PHE | B | 1838 | −13.535 | −45.830 | 79.502 | 1.00 | 197.03 | O |
| ATOM | 6234 | CB | PHE | B | 1838 | −16.597 | −44.162 | 79.402 | 1.00 | 196.74 | C |
| ATOM | 6235 | CG | PHE | B | 1838 | −17.172 | −45.328 | 80.156 | 1.00 | 197.01 | C |
| ATOM | 6236 | CD1 | PHE | B | 1838 | −17.772 | −46.393 | 79.477 | 1.00 | 196.82 | C |
| ATOM | 6237 | CD2 | PHE | B | 1838 | −17.149 | −45.342 | 81.551 | 1.00 | 197.57 | C |
| ATOM | 6238 | CE1 | PHE | B | 1838 | −18.319 | −47.464 | 80.169 | 1.00 | 197.40 | C |
| ATOM | 6239 | CE2 | PHE | B | 1838 | −17.698 | −46.411 | 82.261 | 1.00 | 198.00 | C |
| ATOM | 6240 | CZ | PHE | B | 1838 | −18.284 | −47.476 | 81.569 | 1.00 | 197.84 | C |
| ATOM | 6241 | N | SER | B | 1839 | −13.846 | −43.811 | 80.450 | 1.00 | 196.94 | N |
| ATOM | 6242 | CA | SER | B | 1839 | −12.820 | −44.057 | 81.453 | 1.00 | 197.14 | C |
| ATOM | 6243 | C | SER | B | 1839 | −13.358 | −44.848 | 82.633 | 1.00 | 197.43 | C |
| ATOM | 6244 | O | SER | B | 1839 | −14.350 | −44.463 | 83.260 | 1.00 | 197.21 | O |
| ATOM | 6245 | CB | SER | B | 1839 | −12.171 | −42.771 | 81.930 | 1.00 | 197.11 | C |
| ATOM | 6246 | OG | SER | B | 1839 | −11.044 | −43.090 | 82.723 | 1.00 | 196.96 | O |
| ATOM | 6247 | N | ASP | B | 1840 | −12.689 | −45.961 | 82.918 | 1.00 | 198.03 | N |
| ATOM | 6248 | CA | ASP | B | 1840 | −13.097 | −46.868 | 83.991 | 1.00 | 198.84 | C |
| ATOM | 6249 | C | ASP | B | 1840 | −12.295 | −46.670 | 85.280 | 1.00 | 199.10 | C |
| ATOM | 6250 | O | ASP | B | 1840 | −12.492 | −47.398 | 86.263 | 1.00 | 199.19 | O |
| ATOM | 6251 | CB | ASP | B | 1840 | −13.094 | −48.347 | 83.525 | 1.00 | 199.09 | C |
| ATOM | 6252 | CG | ASP | B | 1840 | −11.708 | −49.028 | 83.612 | 1.00 | 199.98 | C |
| ATOM | 6253 | OD1 | ASP | B | 1840 | −11.678 | −50.269 | 83.779 | 1.00 | 200.88 | O |
| ATOM | 6254 | OD2 | ASP | B | 1840 | −10.657 | −48.354 | 83.504 | 1.00 | 201.07 | O |
| ATOM | 6255 | N | VAL | B | 1841 | −11.394 | −45.685 | 85.267 | 1.00 | 199.40 | N |
| ATOM | 6256 | CA | VAL | B | 1841 | −10.683 | −45.268 | 86.475 | 1.00 | 199.56 | C |
| ATOM | 6257 | C | VAL | B | 1841 | −11.721 | −44.902 | 87.531 | 1.00 | 199.76 | C |
| ATOM | 6258 | O | VAL | B | 1841 | −11.677 | −45.419 | 88.644 | 1.00 | 199.97 | O |
| ATOM | 6259 | CB | VAL | B | 1841 | −9.665 | −44.130 | 86.194 | 1.00 | 199.52 | C |
| ATOM | 6260 | CG1 | VAL | B | 1841 | −9.545 | −43.164 | 87.376 | 1.00 | 199.47 | C |
| ATOM | 6261 | CG2 | VAL | B | 1841 | −8.310 | −44.730 | 85.840 | 1.00 | 199.51 | C |
| ATOM | 6262 | N | ASP | B | 1842 | −12.674 | −44.048 | 87.169 | 1.00 | 199.91 | N |
| ATOM | 6263 | CA | ASP | B | 1842 | −13.857 | −43.864 | 87.998 | 1.00 | 200.07 | C |
| ATOM | 6264 | C | ASP | B | 1842 | −15.123 | −43.694 | 87.166 | 1.00 | 200.04 | C |
| ATOM | 6265 | O | ASP | B | 1842 | −15.677 | −42.597 | 87.067 | 1.00 | 200.07 | O |
| ATOM | 6266 | CB | ASP | B | 1842 | −13.686 | −42.709 | 88.985 | 1.00 | 200.20 | C |
| ATOM | 6267 | CG | ASP | B | 1842 | −14.603 | −42.841 | 90.186 | 1.00 | 200.81 | C |
| ATOM | 6268 | OD1 | ASP | B | 1842 | −14.553 | −41.954 | 91.068 | 1.00 | 201.47 | O |
| ATOM | 6269 | OD2 | ASP | B | 1842 | −15.368 | −43.837 | 90.250 | 1.00 | 201.43 | O |
| ATOM | 6270 | N | LEU | B | 1843 | −15.565 | −44.806 | 86.581 | 1.00 | 200.02 | N |
| ATOM | 6271 | CA | LEU | B | 1843 | −16.815 | −44.909 | 85.805 | 1.00 | 199.82 | C |
| ATOM | 6272 | C | LEU | B | 1843 | −17.961 | −44.015 | 86.312 | 1.00 | 199.49 | C |
| ATOM | 6273 | O | LEU | B | 1843 | −18.795 | −43.559 | 85.521 | 1.00 | 199.50 | O |
| ATOM | 6274 | CB | LEU | B | 1843 | −17.262 | −46.386 | 85.709 | 1.00 | 199.96 | C |
| ATOM | 6275 | CG | LEU | B | 1843 | −16.644 | −47.439 | 86.658 | 1.00 | 200.07 | C |
| ATOM | 6276 | CD1 | LEU | B | 1843 | −17.189 | −47.322 | 88.096 | 1.00 | 199.54 | C |
| ATOM | 6277 | CD2 | LEU | B | 1843 | −16.762 | −48.880 | 86.100 | 1.00 | 199.58 | C |
| ATOM | 6278 | N | GLU | B | 1844 | −17.978 | −43.770 | 87.624 | 1.00 | 198.95 | N |
| ATOM | 6279 | CA | GLU | B | 1844 | −18.934 | −42.867 | 88.267 | 1.00 | 198.38 | C |
| ATOM | 6280 | C | GLU | B | 1844 | −18.654 | −41.398 | 87.920 | 1.00 | 197.80 | C |
| ATOM | 6281 | O | GLU | B | 1844 | −19.502 | −40.720 | 87.339 | 1.00 | 197.61 | O |
| ATOM | 6282 | CB | GLU | B | 1844 | −18.917 | −43.089 | 89.789 | 1.00 | 198.50 | C |
| ATOM | 6283 | CG | GLU | B | 1844 | −20.106 | −42.494 | 90.561 | 1.00 | 198.92 | C |
| ATOM | 6284 | CD | GLU | B | 1844 | −19.734 | −41.308 | 91.451 | 1.00 | 199.77 | C |
| ATOM | 6285 | OE1 | GLU | B | 1844 | −18.596 | −41.269 | 91.976 | 1.00 | 200.13 | O |
| ATOM | 6286 | OE2 | GLU | B | 1844 | −20.593 | −40.417 | 91.645 | 1.00 | 200.06 | O |
| ATOM | 6287 | N | LYS | B | 1845 | −17.465 | −40.919 | 88.274 | 1.00 | 197.24 | N |
| ATOM | 6288 | CA | LYS | B | 1845 | −17.102 | −39.520 | 88.054 | 1.00 | 196.80 | C |
| ATOM | 6289 | C | LYS | B | 1845 | −16.684 | −39.248 | 86.609 | 1.00 | 196.78 | C |
| ATOM | 6290 | O | LYS | B | 1845 | −17.134 | −38.264 | 86.016 | 1.00 | 196.75 | O |
| ATOM | 6291 | CB | LYS | B | 1845 | −16.009 | −39.058 | 89.037 | 1.00 | 196.69 | C |
| ATOM | 6292 | CG | LYS | B | 1845 | −16.505 | −38.831 | 90.456 | 1.00 | 195.56 | C |
| ATOM | 6293 | CD | LYS | B | 1845 | −15.509 | −38.090 | 91.312 | 1.00 | 193.69 | C |
| ATOM | 6294 | CE | LYS | B | 1845 | −16.131 | −37.817 | 92.663 | 1.00 | 193.05 | C |
| ATOM | 6295 | NZ | LYS | B | 1845 | −15.203 | −37.146 | 93.600 | 1.00 | 192.76 | N |
| ATOM | 6296 | N | ASP | B | 1846 | −15.844 | −40.128 | 86.050 | 1.00 | 196.69 | N |
| ATOM | 6297 | CA | ASP | B | 1846 | −15.230 | −39.962 | 84.710 | 1.00 | 196.50 | C |
| ATOM | 6298 | C | ASP | B | 1846 | −16.237 | −40.049 | 83.546 | 1.00 | 196.20 | C |
| ATOM | 6299 | O | ASP | B | 1846 | −15.862 | −40.275 | 82.390 | 1.00 | 196.10 | O |
| ATOM | 6300 | CB | ASP | B | 1846 | −14.066 | −40.956 | 84.508 | 1.00 | 196.60 | C |
| ATOM | 6301 | CG | ASP | B | 1846 | −12.930 | −40.781 | 85.537 | 1.00 | 197.02 | C |
| ATOM | 6302 | OD1 | ASP | B | 1846 | −12.927 | −39.793 | 86.309 | 1.00 | 197.69 | O |
| ATOM | 6303 | OD2 | ASP | B | 1846 | −12.026 | −41.647 | 85.573 | 1.00 | 197.34 | O |
| ATOM | 6304 | N | VAL | B | 1847 | −17.514 | −39.870 | 83.887 | 1.00 | 195.90 | N |
| ATOM | 6305 | CA | VAL | B | 1847 | −18.638 | −39.846 | 82.945 | 1.00 | 195.41 | C |
| ATOM | 6306 | C | VAL | B | 1847 | −19.333 | −38.455 | 83.004 | 1.00 | 194.98 | C |
| ATOM | 6307 | O | VAL | B | 1847 | −19.781 | −37.922 | 81.984 | 1.00 | 194.84 | O |
| ATOM | 6308 | CB | VAL | B | 1847 | −19.615 | −41.082 | 83.179 | 1.00 | 195.44 | C |
| ATOM | 6309 | CG1 | VAL | B | 1847 | −20.757 | −40.779 | 84.171 | 1.00 | 195.43 | C |
| ATOM | 6310 | CG2 | VAL | B | 1847 | −20.174 | −41.597 | 81.869 | 1.00 | 195.41 | C |
| ATOM | 6311 | N | HIS | B | 1848 | −19.396 | −37.872 | 84.205 | 1.00 | 194.42 | N |
| ATOM | 6312 | CA | HIS | B | 1848 | −19.819 | −36.484 | 84.386 | 1.00 | 193.57 | C |
| ATOM | 6313 | C | HIS | B | 1848 | −18.622 | −35.594 | 84.053 | 1.00 | 193.30 | C |
| ATOM | 6314 | O | HIS | B | 1848 | −18.779 | −34.447 | 83.634 | 1.00 | 193.29 | O |
| ATOM | 6315 | CB | HIS | B | 1848 | −20.282 | −36.229 | 85.828 | 1.00 | 193.34 | C |
| ATOM | 6316 | CG | HIS | B | 1848 | −21.352 | −37.161 | 86.308 | 1.00 | 192.16 | C |
| ATOM | 6317 | ND1 | HIS | B | 1848 | −22.686 | −36.976 | 86.019 | 1.00 | 191.23 | N |
| ATOM | 6318 | CD2 | HIS | B | 1848 | −21.284 | −38.276 | 87.074 | 1.00 | 191.17 | C |
| ATOM | 6319 | CE1 | HIS | B | 1848 | −23.393 | −37.943 | 86.577 | 1.00 | 190.93 | C |
| ATOM | 6320 | NE2 | HIS | B | 1848 | −22.566 | −38.745 | 87.223 | 1.00 | 190.66 | N |
| ATOM | 6321 | N | SER | B | 1849 | −17.428 | −36.149 | 84.246 | 1.00 | 192.85 | N |
| ATOM | 6322 | CA | SER | B | 1849 | −16.178 | −35.483 | 83.934 | 1.00 | 192.56 | C |
| ATOM | 6323 | C | SER | B | 1849 | −16.091 | −35.179 | 82.462 | 1.00 | 192.43 | C |
| ATOM | 6324 | O | SER | B | 1849 | −15.824 | −34.047 | 82.078 | 1.00 | 192.46 | O |
| ATOM | 6325 | CB | SER | B | 1849 | −15.016 | −36.380 | 84.308 | 1.00 | 192.60 | C |
| ATOM | 6326 | OG | SER | B | 1849 | −15.157 | −36.834 | 85.638 | 1.00 | 192.64 | O |
| ATOM | 6327 | N | GLY | B | 1850 | −16.299 | −36.199 | 81.637 | 1.00 | 192.21 | N |
| ATOM | 6328 | CA | GLY | B | 1850 | −16.371 | −35.990 | 80.202 | 1.00 | 192.15 | C |
| ATOM | 6329 | C | GLY | B | 1850 | −15.552 | −36.934 | 79.351 | 1.00 | 192.08 | C |
| ATOM | 6330 | O | GLY | B | 1850 | −14.810 | −36.517 | 78.462 | 1.00 | 192.08 | O |
| ATOM | 6331 | N | LEU | B | 1851 | −15.692 | −38.222 | 79.604 | 1.00 | 192.08 | N |
| ATOM | 6332 | CA | LEU | B | 1851 | −15.039 | −39.194 | 78.754 | 1.00 | 192.00 | C |
| ATOM | 6333 | C | LEU | B | 1851 | −16.062 | −40.192 | 78.205 | 1.00 | 191.87 | C |
| ATOM | 6334 | O | LEU | B | 1851 | −15.891 | −41.401 | 78.342 | 1.00 | 191.91 | O |
| ATOM | 6335 | CB | LEU | B | 1851 | −13.868 | −39.863 | 79.494 | 1.00 | 192.09 | C |
| ATOM | 6336 | CG | LEU | B | 1851 | −12.851 | −38.875 | 80.093 | 1.00 | 192.16 | C |
| ATOM | 6337 | CD1 | LEU | B | 1851 | −12.993 | −38.813 | 81.607 | 1.00 | 192.09 | C |
| ATOM | 6338 | CD2 | LEU | B | 1851 | −11.417 | −39.211 | 79.712 | 1.00 | 192.08 | C |
| ATOM | 6339 | N | ILE | B | 1852 | −17.140 | −39.660 | 77.618 | 1.00 | 191.62 | N |
| ATOM | 6340 | CA | ILE | B | 1852 | −18.123 | −40.446 | 76.864 | 1.00 | 191.35 | C |
| ATOM | 6341 | C | ILE | B | 1852 | −17.483 | −40.688 | 75.499 | 1.00 | 191.52 | C |
| ATOM | 6342 | O | ILE | B | 1852 | −16.800 | −39.808 | 74.969 | 1.00 | 191.54 | O |
| ATOM | 6343 | CB | ILE | B | 1852 | −19.493 | −39.681 | 76.664 | 1.00 | 191.40 | C |
| ATOM | 6344 | CG1 | ILE | B | 1852 | −20.520 | −39.936 | 77.789 | 1.00 | 191.14 | C |
| ATOM | 6345 | CG2 | ILE | B | 1852 | −20.145 | −40.086 | 75.362 | 1.00 | 191.89 | C |
| ATOM | 6346 | CD1 | ILE | B | 1852 | −21.921 | −39.200 | 77.611 | 1.00 | 190.46 | C |
| ATOM | 6347 | N | GLY | B | 1853 | −17.695 | −41.879 | 74.939 | 1.00 | 191.63 | N |
| ATOM | 6348 | CA | GLY | B | 1853 | −17.190 | −42.211 | 73.605 | 1.00 | 191.81 | C |
| ATOM | 6349 | C | GLY | B | 1853 | −17.989 | −43.274 | 72.854 | 1.00 | 191.95 | C |
| ATOM | 6350 | O | GLY | B | 1853 | −18.436 | −44.257 | 73.464 | 1.00 | 191.98 | O |
| ATOM | 6351 | N | PRO | B | 1854 | −18.161 | −43.090 | 71.518 | 1.00 | 191.93 | N |
| ATOM | 6352 | CA | PRO | B | 1854 | −18.935 | −43.975 | 70.638 | 1.00 | 191.63 | C |
| ATOM | 6353 | C | PRO | B | 1854 | −18.096 | −45.019 | 69.888 | 1.00 | 191.28 | C |
| ATOM | 6354 | O | PRO | B | 1854 | −16.966 | −44.752 | 69.476 | 1.00 | 191.37 | O |
| ATOM | 6355 | CB | PRO | B | 1854 | −19.547 | −42.993 | 69.638 | 1.00 | 191.67 | C |
| ATOM | 6356 | CG | PRO | B | 1854 | −18.526 | −41.856 | 69.542 | 1.00 | 191.99 | C |
| ATOM | 6357 | CD | PRO | B | 1854 | −17.604 | −41.954 | 70.755 | 1.00 | 192.08 | C |
| ATOM | 6358 | N | LEU | B | 1855 | −18.664 | −46.198 | 69.692 | 1.00 | 190.80 | N |
| ATOM | 6359 | CA | LEU | B | 1855 | −17.946 | −47.264 | 69.047 | 1.00 | 190.41 | C |
| ATOM | 6360 | C | LEU | B | 1855 | −18.906 | −48.041 | 68.169 | 1.00 | 190.56 | C |
| ATOM | 6361 | O | LEU | B | 1855 | −19.976 | −48.454 | 68.624 | 1.00 | 190.67 | O |
| ATOM | 6362 | CB | LEU | B | 1855 | −17.352 | −48.161 | 70.107 | 1.00 | 190.17 | C |
| ATOM | 6363 | CG | LEU | B | 1855 | −16.005 | −48.760 | 69.779 | 1.00 | 189.81 | C |
| ATOM | 6364 | CD1 | LEU | B | 1855 | −15.508 | −49.428 | 71.018 | 1.00 | 189.75 | C |
| ATOM | 6365 | CD2 | LEU | B | 1855 | −16.134 | −49.771 | 68.676 | 1.00 | 190.37 | C |
| ATOM | 6366 | N | LEU | B | 1856 | −18.527 | −48.234 | 66.909 | 1.00 | 190.61 | N |
| ATOM | 6367 | CA | LEU | B | 1856 | −19.423 | −48.831 | 65.916 | 1.00 | 190.53 | C |
| ATOM | 6368 | C | LEU | B | 1856 | −18.871 | −50.130 | 65.344 | 1.00 | 190.54 | C |
| ATOM | 6369 | O | LEU | B | 1856 | −17.719 | −50.204 | 64.941 | 1.00 | 190.38 | O |
| ATOM | 6370 | CB | LEU | B | 1856 | −19.722 | −47.824 | 64.797 | 1.00 | 190.56 | C |
| ATOM | 6371 | CG | LEU | B | 1856 | −20.912 | −46.853 | 64.914 | 1.00 | 190.28 | C |
| ATOM | 6372 | CD1 | LEU | B | 1856 | −21.036 | −46.186 | 66.277 | 1.00 | 189.62 | C |
| ATOM | 6373 | CD2 | LEU | B | 1856 | −20.844 | −45.795 | 63.819 | 1.00 | 190.30 | C |
| ATOM | 6374 | N | VAL | B | 1857 | −19.714 | −51.149 | 65.308 | 1.00 | 190.80 | N |
| ATOM | 6375 | CA | VAL | B | 1857 | −19.290 | −52.479 | 64.915 | 1.00 | 191.47 | C |
| ATOM | 6376 | C | VAL | B | 1857 | −20.156 | −52.954 | 63.765 | 1.00 | 192.12 | C |
| ATOM | 6377 | O | VAL | B | 1857 | −21.357 | −53.126 | 63.940 | 1.00 | 192.32 | O |
| ATOM | 6378 | CB | VAL | B | 1857 | −19.415 | −53.446 | 66.106 | 1.00 | 191.43 | C |
| ATOM | 6379 | CG1 | VAL | B | 1857 | −19.262 | −54.903 | 65.665 | 1.00 | 191.30 | C |
| ATOM | 6380 | CG2 | VAL | B | 1857 | −18.399 | −53.083 | 67.177 | 1.00 | 191.12 | C |
| ATOM | 6381 | N | CYS | B | 1858 | −19.552 | −53.177 | 62.599 | 1.00 | 192.94 | N |
| ATOM | 6382 | CA | CYS | B | 1858 | −20.311 | −53.361 | 61.353 | 1.00 | 194.08 | C |
| ATOM | 6383 | C | CYS | B | 1858 | −20.202 | −54.729 | 60.674 | 1.00 | 194.48 | C |
| ATOM | 6384 | O | CYS | B | 1858 | −19.223 | −55.437 | 60.870 | 1.00 | 194.61 | O |
| ATOM | 6385 | CB | CYS | B | 1858 | −19.911 | −52.279 | 60.357 | 1.00 | 194.18 | C |
| ATOM | 6386 | SG | CYS | B | 1858 | −20.842 | −50.715 | 60.475 | 1.00 | 196.29 | S |
| ATOM | 6387 | N | HIS | B | 1859 | −21.214 | −55.083 | 59.873 | 1.00 | 195.19 | N |
| ATOM | 6388 | CA | HIS | B | 1859 | −21.179 | −56.277 | 59.013 | 1.00 | 196.03 | C |
| ATOM | 6389 | C | HIS | B | 1859 | −20.016 | −56.147 | 58.057 | 1.00 | 196.25 | C |
| ATOM | 6390 | O | HIS | B | 1859 | −19.582 | −55.031 | 57.768 | 1.00 | 196.46 | O |
| ATOM | 6391 | CB | HIS | B | 1859 | −22.424 | −56.357 | 58.139 | 1.00 | 196.41 | C |
| ATOM | 6392 | CG | HIS | B | 1859 | −23.647 | −56.863 | 58.835 | 1.00 | 197.82 | C |
| ATOM | 6393 | ND1 | HIS | B | 1859 | −24.693 | −56.036 | 59.193 | 1.00 | 198.69 | N |
| ATOM | 6394 | CD2 | HIS | B | 1859 | −24.016 | −58.117 | 59.191 | 1.00 | 198.86 | C |
| ATOM | 6395 | CE1 | HIS | B | 1859 | −25.643 | −56.756 | 59.762 | 1.00 | 199.27 | C |
| ATOM | 6396 | NE2 | HIS | B | 1859 | −25.257 | −58.021 | 59.776 | 1.00 | 199.63 | N |
| ATOM | 6397 | N | THR | B | 1860 | −19.528 | −57.271 | 57.537 | 1.00 | 196.53 | N |
| ATOM | 6398 | CA | THR | B | 1860 | −18.438 | −57.217 | 56.564 | 1.00 | 196.65 | C |
| ATOM | 6399 | C | THR | B | 1860 | −18.939 | −56.624 | 55.252 | 1.00 | 197.03 | C |
| ATOM | 6400 | O | THR | B | 1860 | −20.100 | −56.814 | 54.880 | 1.00 | 196.81 | O |
| ATOM | 6401 | CB | THR | B | 1860 | −17.706 | −58.567 | 56.373 | 1.00 | 196.52 | C |
| ATOM | 6402 | OG1 | THR | B | 1860 | −18.613 | −59.648 | 56.614 | 1.00 | 196.44 | O |
| ATOM | 6403 | CG2 | THR | B | 1860 | −16.525 | −58.680 | 57.343 | 1.00 | 195.99 | C |
| ATOM | 6404 | N | ASN | B | 1861 | −18.056 | −55.875 | 54.593 | 1.00 | 197.70 | N |
| ATOM | 6405 | CA | ASN | B | 1861 | −18.371 | −55.134 | 53.375 | 1.00 | 198.53 | C |
| ATOM | 6406 | C | ASN | B | 1861 | −19.447 | −54.067 | 53.600 | 1.00 | 198.82 | C |
| ATOM | 6407 | O | ASN | B | 1861 | −20.460 | −54.040 | 52.903 | 1.00 | 198.80 | O |
| ATOM | 6408 | CB | ASN | B | 1861 | −18.761 | −56.098 | 52.240 | 1.00 | 198.83 | C |
| ATOM | 6409 | CG | ASN | B | 1861 | −18.481 | −55.530 | 50.846 | 1.00 | 199.86 | C |
| ATOM | 6410 | OD1 | ASN | B | 1861 | −18.295 | −56.284 | 49.882 | 1.00 | 200.67 | O |
| ATOM | 6411 | ND2 | ASN | B | 1861 | −18.453 | −54.203 | 50.732 | 1.00 | 201.03 | N |
| ATOM | 6412 | N | THR | B | 1862 | −19.220 | −53.196 | 54.581 | 1.00 | 199.44 | N |
| ATOM | 6413 | CA | THR | B | 1862 | −20.140 | −52.087 | 54.875 | 1.00 | 200.21 | C |
| ATOM | 6414 | C | THR | B | 1862 | −19.435 | −50.736 | 54.981 | 1.00 | 200.89 | C |
| ATOM | 6415 | O | THR | B | 1862 | −20.070 | −49.684 | 54.836 | 1.00 | 200.92 | O |
| ATOM | 6416 | CB | THR | B | 1862 | −20.933 | −52.310 | 56.174 | 1.00 | 200.08 | C |
| ATOM | 6417 | OG1 | THR | B | 1862 | −20.047 | −52.755 | 57.204 | 1.00 | 200.00 | O |
| ATOM | 6418 | CG2 | THR | B | 1862 | −22.037 | −53.326 | 55.967 | 1.00 | 200.22 | C |
| ATOM | 6419 | N | LEU | B | 1863 | −18.131 | −50.771 | 55.250 | 1.00 | 201.78 | N |
| ATOM | 6420 | CA | LEU | B | 1863 | −17.307 | −49.561 | 55.284 | 1.00 | 202.65 | C |
| ATOM | 6421 | C | LEU | B | 1863 | −16.707 | −49.258 | 53.896 | 1.00 | 203.55 | C |
| ATOM | 6422 | O | LEU | B | 1863 | −17.200 | −49.758 | 52.883 | 1.00 | 203.74 | O |
| ATOM | 6423 | CB | LEU | B | 1863 | −16.232 | −49.665 | 56.378 | 1.00 | 202.43 | C |
| ATOM | 6424 | CG | LEU | B | 1863 | −16.659 | −50.009 | 57.817 | 1.00 | 201.92 | C |
| ATOM | 6425 | CD1 | LEU | B | 1863 | −15.512 | −49.748 | 58.775 | 1.00 | 201.45 | C |
| ATOM | 6426 | CD2 | LEU | B | 1863 | −17.898 | −49.246 | 58.277 | 1.00 | 201.25 | C |
| ATOM | 6427 | N | ASN | B | 1864 | −15.659 | −48.443 | 53.836 | 1.00 | 204.65 | N |
| ATOM | 6428 | CA | ASN | B | 1864 | −15.175 | −47.950 | 52.552 | 1.00 | 205.82 | C |
| ATOM | 6429 | C | ASN | B | 1864 | −13.691 | −47.675 | 52.511 | 1.00 | 206.64 | C |
| ATOM | 6430 | O | ASN | B | 1864 | −13.007 | −47.806 | 53.521 | 1.00 | 206.78 | O |
| ATOM | 6431 | CB | ASN | B | 1864 | −15.911 | −46.656 | 52.195 | 1.00 | 205.88 | C |
| ATOM | 6432 | CG | ASN | B | 1864 | −17.098 | −46.893 | 51.294 | 1.00 | 206.60 | C |
| ATOM | 6433 | OD1 | ASN | B | 1864 | −18.209 | −46.419 | 51.567 | 1.00 | 207.04 | O |
| ATOM | 6434 | ND2 | ASN | B | 1864 | −16.876 | −47.632 | 50.205 | 1.00 | 207.17 | N |
| ATOM | 6435 | N | PRO | B | 1865 | −13.177 | −47.320 | 51.325 | 1.00 | 207.52 | N |
| ATOM | 6436 | CA | PRO | B | 1865 | −11.951 | −46.528 | 51.307 | 1.00 | 208.25 | C |
| ATOM | 6437 | C | PRO | B | 1865 | −12.169 | −45.116 | 51.884 | 1.00 | 209.04 | C |
| ATOM | 6438 | O | PRO | B | 1865 | −11.261 | −44.574 | 52.518 | 1.00 | 208.92 | O |
| ATOM | 6439 | CB | PRO | B | 1865 | −11.593 | −46.460 | 49.816 | 1.00 | 208.24 | C |
| ATOM | 6440 | CG | PRO | B | 1865 | −12.871 | −46.742 | 49.092 | 1.00 | 208.08 | C |
| ATOM | 6441 | CD | PRO | B | 1865 | −13.638 | −47.674 | 49.970 | 1.00 | 207.57 | C |
| ATOM | 6442 | N | ALA | B | 1866 | −13.365 | −44.547 | 51.689 | 1.00 | 210.01 | N |
| ATOM | 6443 | CA | ALA | B | 1866 | −13.656 | −43.154 | 52.072 | 1.00 | 211.23 | C |
| ATOM | 6444 | C | ALA | B | 1866 | −13.761 | −42.908 | 53.585 | 1.00 | 211.65 | C |
| ATOM | 6445 | O | ALA | B | 1866 | −14.172 | −41.817 | 53.985 | 1.00 | 211.85 | O |
| ATOM | 6446 | CB | ALA | B | 1866 | −14.915 | −42.620 | 51.337 | 1.00 | 210.55 | C |
| ATOM | 6447 | N | HIS | B | 1867 | −13.357 | −43.903 | 54.396 | 1.00 | 212.73 | N |
| ATOM | 6448 | CA | HIS | B | 1867 | −13.471 | −43.935 | 55.896 | 1.00 | 212.88 | C |
| ATOM | 6449 | C | HIS | B | 1867 | −14.924 | −43.933 | 56.411 | 1.00 | 213.23 | C |
| ATOM | 6450 | O | HIS | B | 1867 | −15.759 | −43.144 | 55.964 | 1.00 | 213.43 | O |
| ATOM | 6451 | CB | HIS | B | 1867 | −12.611 | −42.836 | 56.602 | 1.00 | 213.59 | C |
| ATOM | 6452 | CG | HIS | B | 1867 | −12.478 | −42.987 | 58.111 | 1.00 | 214.35 | C |
| ATOM | 6453 | ND1 | HIS | B | 1867 | −11.256 | −42.964 | 58.759 | 1.00 | 216.57 | N |
| ATOM | 6454 | CD2 | HIS | B | 1867 | −13.411 | −43.123 | 59.093 | 1.00 | 216.21 | C |
| ATOM | 6455 | CE1 | HIS | B | 1867 | −11.440 | −43.094 | 60.065 | 1.00 | 216.57 | C |
| ATOM | 6456 | NE2 | HIS | B | 1867 | −12.739 | −43.194 | 60.294 | 1.00 | 216.31 | N |
| ATOM | 6457 | N | GLY | B | 1868 | −15.216 | −44.807 | 57.369 | 1.00 | 213.26 | N |
| ATOM | 6458 | CA | GLY | B | 1868 | −16.575 | −44.945 | 57.871 | 1.00 | 213.58 | C |
| ATOM | 6459 | C | GLY | B | 1868 | −17.410 | −45.607 | 56.798 | 1.00 | 213.89 | C |
| ATOM | 6460 | O | GLY | B | 1868 | −16.868 | −46.243 | 55.890 | 1.00 | 214.01 | O |
| ATOM | 6461 | N | ARG | B | 1869 | −18.725 | −45.463 | 56.889 | 1.00 | 214.13 | N |
| ATOM | 6462 | CA | ARG | B | 1869 | −19.611 | −45.990 | 55.853 | 1.00 | 214.47 | C |
| ATOM | 6463 | C | ARG | B | 1869 | −20.432 | −44.859 | 55.262 | 1.00 | 214.41 | C |
| ATOM | 6464 | O | ARG | B | 1869 | −20.281 | −43.704 | 55.683 | 1.00 | 214.59 | O |
| ATOM | 6465 | CB | ARG | B | 1869 | −20.519 | −47.080 | 56.410 | 1.00 | 214.56 | C |
| ATOM | 6466 | CG | ARG | B | 1869 | −21.362 | −46.657 | 57.597 | 1.00 | 215.67 | C |
| ATOM | 6467 | CD | ARG | B | 1869 | −21.958 | −47.872 | 58.280 | 1.00 | 217.79 | C |
| ATOM | 6468 | NE | ARG | B | 1869 | −23.296 | −48.224 | 57.794 | 1.00 | 219.19 | N |
| ATOM | 6469 | CZ | ARG | B | 1869 | −23.567 | −48.919 | 56.688 | 1.00 | 219.89 | C |
| ATOM | 6470 | NH1 | ARG | B | 1869 | −24.831 | −49.171 | 56.377 | 1.00 | 220.30 | N |
| ATOM | 6471 | NH2 | ARG | B | 1869 | −22.599 | −49.348 | 55.881 | 1.00 | 219.85 | N |
| ATOM | 6472 | N | GLN | B | 1870 | −21.288 | −45.184 | 54.290 | 1.00 | 214.16 | N |
| ATOM | 6473 | CA | GLN | B | 1870 | −22.160 | −44.178 | 53.689 | 1.00 | 213.82 | C |
| ATOM | 6474 | C | GLN | B | 1870 | −23.171 | −44.716 | 52.674 | 1.00 | 213.48 | C |
| ATOM | 6475 | O | GLN | B | 1870 | −23.968 | −43.945 | 52.152 | 1.00 | 213.15 | O |
| ATOM | 6476 | CB | GLN | B | 1870 | −21.327 | −43.038 | 53.107 | 1.00 | 213.84 | C |
| ATOM | 6477 | CG | GLN | B | 1870 | −20.492 | −43.410 | 51.914 | 1.00 | 214.49 | C |
| ATOM | 6478 | CD | GLN | B | 1870 | −20.932 | −42.648 | 50.689 | 1.00 | 216.24 | C |
| ATOM | 6479 | OE1 | GLN | B | 1870 | −22.099 | −42.250 | 50.579 | 1.00 | 217.13 | O |
| ATOM | 6480 | NE2 | GLN | B | 1870 | −19.998 | −42.411 | 49.764 | 1.00 | 216.97 | N |
| ATOM | 6481 | N | VAL | B | 1871 | −23.149 | −46.030 | 52.427 | 1.00 | 213.43 | N |
| ATOM | 6482 | CA | VAL | B | 1871 | −24.083 | −46.695 | 51.489 | 1.00 | 213.41 | C |
| ATOM | 6483 | C | VAL | B | 1871 | −25.532 | −46.385 | 51.871 | 1.00 | 213.20 | C |
| ATOM | 6484 | O | VAL | B | 1871 | −26.199 | −47.167 | 52.561 | 1.00 | 213.02 | O |
| ATOM | 6485 | CB | VAL | B | 1871 | −23.816 | −48.252 | 51.334 | 1.00 | 213.61 | C |
| ATOM | 6486 | CG1 | VAL | B | 1871 | −24.983 | −48.976 | 50.619 | 1.00 | 213.45 | C |
| ATOM | 6487 | CG2 | VAL | B | 1871 | −22.481 | −48.527 | 50.595 | 1.00 | 213.79 | C |
| ATOM | 6488 | N | THR | B | 1872 | −25.991 | −45.229 | 51.382 | 1.00 | 213.18 | N |
| ATOM | 6489 | CA | THR | B | 1872 | −27.236 | −44.544 | 51.821 | 1.00 | 213.07 | C |
| ATOM | 6490 | C | THR | B | 1872 | −27.311 | −44.449 | 53.374 | 1.00 | 212.25 | C |
| ATOM | 6491 | O | THR | B | 1872 | −28.320 | −44.817 | 54.000 | 1.00 | 212.34 | O |
| ATOM | 6492 | CB | THR | B | 1872 | −28.571 | −45.099 | 51.111 | 1.00 | 213.43 | C |
| ATOM | 6493 | OG1 | THR | B | 1872 | −28.325 | −45.393 | 49.721 | 1.00 | 214.05 | O |
| ATOM | 6494 | CG2 | THR | B | 1872 | −29.743 | −44.079 | 51.204 | 1.00 | 213.28 | C |
| ATOM | 6495 | N | VAL | B | 1873 | −26.231 | −43.951 | 53.983 | 1.00 | 210.96 | N |
| ATOM | 6496 | CA | VAL | B | 1873 | −26.128 | −43.965 | 55.443 | 1.00 | 209.56 | C |
| ATOM | 6497 | C | VAL | B | 1873 | −26.357 | −42.594 | 56.083 | 1.00 | 208.46 | C |
| ATOM | 6498 | O | VAL | B | 1873 | −27.505 | −42.181 | 56.241 | 1.00 | 208.32 | O |
| ATOM | 6499 | CB | VAL | B | 1873 | −24.826 | −44.658 | 55.948 | 1.00 | 209.66 | C |
| ATOM | 6500 | CG1 | VAL | B | 1873 | −24.952 | −45.001 | 57.407 | 1.00 | 209.80 | C |
| ATOM | 6501 | CG2 | VAL | B | 1873 | −24.577 | −45.941 | 55.198 | 1.00 | 209.62 | C |
| ATOM | 6502 | N | GLN | B | 1874 | −25.277 | −41.894 | 56.437 | 1.00 | 207.04 | N |
| ATOM | 6503 | CA | GLN | B | 1874 | −25.358 | −40.654 | 57.207 | 1.00 | 205.63 | C |
| ATOM | 6504 | C | GLN | B | 1874 | −25.782 | −40.977 | 58.628 | 1.00 | 204.53 | C |
| ATOM | 6505 | O | GLN | B | 1874 | −26.970 | −41.134 | 58.932 | 1.00 | 204.17 | O |
| ATOM | 6506 | CB | GLN | B | 1874 | −26.334 | −39.680 | 56.566 | 1.00 | 205.81 | C |
| ATOM | 6507 | CG | GLN | B | 1874 | −26.023 | −38.228 | 56.797 | 1.00 | 206.39 | C |
| ATOM | 6508 | CD | GLN | B | 1874 | −26.459 | −37.371 | 55.620 | 1.00 | 207.76 | C |
| ATOM | 6509 | OE1 | GLN | B | 1874 | −25.730 | −36.471 | 55.197 | 1.00 | 208.75 | O |
| ATOM | 6510 | NE2 | GLN | B | 1874 | −27.644 | −37.660 | 55.068 | 1.00 | 207.73 | N |
| ATOM | 6511 | N | GLU | B | 1875 | −24.784 | −41.091 | 59.490 | 1.00 | 203.28 | N |
| ATOM | 6512 | CA | GLU | B | 1875 | −24.997 | −41.491 | 60.865 | 1.00 | 202.20 | C |
| ATOM | 6513 | C | GLU | B | 1875 | −24.836 | −40.300 | 61.805 | 1.00 | 201.43 | C |
| ATOM | 6514 | O | GLU | B | 1875 | −24.073 | −39.376 | 61.519 | 1.00 | 201.49 | O |
| ATOM | 6515 | CB | GLU | B | 1875 | −23.958 | −42.535 | 61.260 | 1.00 | 202.31 | C |
| ATOM | 6516 | CG | GLU | B | 1875 | −23.962 | −43.817 | 60.472 | 1.00 | 202.49 | C |
| ATOM | 6517 | CD | GLU | B | 1875 | −22.552 | −44.346 | 60.220 | 1.00 | 202.73 | C |
| ATOM | 6518 | OE1 | GLU | B | 1875 | −21.843 | −43.755 | 59.373 | 1.00 | 202.62 | O |
| ATOM | 6519 | OE2 | GLU | B | 1875 | −22.156 | −45.351 | 60.859 | 1.00 | 202.51 | O |
| ATOM | 6520 | N | PHE | B | 1876 | −25.538 | −40.350 | 62.937 | 1.00 | 200.37 | N |
| ATOM | 6521 | CA | PHE | B | 1876 | −25.314 | −39.438 | 64.067 | 1.00 | 199.21 | C |
| ATOM | 6522 | C | PHE | B | 1876 | −25.444 | −40.136 | 65.439 | 1.00 | 198.42 | C |
| ATOM | 6523 | O | PHE | B | 1876 | −26.336 | −40.961 | 65.656 | 1.00 | 198.26 | O |
| ATOM | 6524 | CB | PHE | B | 1876 | −26.277 | −38.251 | 63.998 | 1.00 | 199.34 | C |
| ATOM | 6525 | CG | PHE | B | 1876 | −26.330 | −37.593 | 62.661 | 1.00 | 199.11 | C |
| ATOM | 6526 | CD1 | PHE | B | 1876 | −25.365 | −36.675 | 62.287 | 1.00 | 199.77 | C |
| ATOM | 6527 | CD2 | PHE | B | 1876 | −27.347 | −37.893 | 61.772 | 1.00 | 199.30 | C |
| ATOM | 6528 | CE1 | PHE | B | 1876 | −25.414 | −36.066 | 61.030 | 1.00 | 200.76 | C |
| ATOM | 6529 | CE2 | PHE | B | 1876 | −27.407 | −37.294 | 60.519 | 1.00 | 199.86 | C |
| ATOM | 6530 | CZ | PHE | B | 1876 | −26.442 | −36.380 | 60.146 | 1.00 | 200.25 | C |
| ATOM | 6531 | N | ALA | B | 1877 | −24.556 | −39.794 | 66.363 | 1.00 | 197.33 | N |
| ATOM | 6532 | CA | ALA | B | 1877 | −24.678 | −40.258 | 67.734 | 1.00 | 196.53 | C |
| ATOM | 6533 | C | ALA | B | 1877 | −25.357 | −39.212 | 68.662 | 1.00 | 196.07 | C |
| ATOM | 6534 | O | ALA | B | 1877 | −25.208 | −38.009 | 68.474 | 1.00 | 195.91 | O |
| ATOM | 6535 | CB | ALA | B | 1877 | −23.313 | −40.656 | 68.255 | 1.00 | 196.42 | C |
| ATOM | 6536 | N | LEU | B | 1878 | −26.120 | −39.676 | 69.649 | 1.00 | 195.54 | N |
| ATOM | 6537 | CA | LEU | B | 1878 | −26.712 | −38.791 | 70.659 | 1.00 | 195.03 | C |
| ATOM | 6538 | C | LEU | B | 1878 | −26.578 | −39.338 | 72.125 | 1.00 | 195.42 | C |
| ATOM | 6539 | O | LEU | B | 1878 | −26.920 | −40.497 | 72.411 | 1.00 | 195.46 | O |
| ATOM | 6540 | CB | LEU | B | 1878 | −28.146 | −38.424 | 70.268 | 1.00 | 194.75 | C |
| ATOM | 6541 | CG | LEU | B | 1878 | −28.387 | −37.103 | 69.534 | 1.00 | 193.82 | C |
| ATOM | 6542 | CD1 | LEU | B | 1878 | −27.707 | −37.040 | 68.212 | 1.00 | 192.82 | C |
| ATOM | 6543 | CD2 | LEU | B | 1878 | −29.871 | −36.895 | 69.337 | 1.00 | 194.44 | C |
| ATOM | 6544 | N | PHE | B | 1879 | −26.121 | −38.468 | 73.039 | 1.00 | 195.52 | N |
| ATOM | 6545 | CA | PHE | B | 1879 | −25.316 | −38.841 | 74.226 | 1.00 | 195.32 | C |
| ATOM | 6546 | C | PHE | B | 1879 | −25.919 | −38.407 | 75.543 | 1.00 | 194.83 | C |
| ATOM | 6547 | O | PHE | B | 1879 | −25.196 | −37.906 | 76.415 | 1.00 | 194.65 | O |
| ATOM | 6548 | CB | PHE | B | 1879 | −23.943 | −38.157 | 74.119 | 1.00 | 195.75 | C |
| ATOM | 6549 | CG | PHE | B | 1879 | −23.617 | −37.633 | 72.724 | 1.00 | 196.46 | C |
| ATOM | 6550 | CD1 | PHE | B | 1879 | −24.364 | −36.595 | 72.154 | 1.00 | 197.02 | C |
| ATOM | 6551 | CD2 | PHE | B | 1879 | −22.567 | −38.176 | 71.986 | 1.00 | 196.87 | C |
| ATOM | 6552 | CE1 | PHE | B | 1879 | −24.083 | −36.116 | 70.870 | 1.00 | 197.27 | C |
| ATOM | 6553 | CE2 | PHE | B | 1879 | −22.271 | −37.698 | 70.704 | 1.00 | 197.41 | C |
| ATOM | 6554 | CZ | PHE | B | 1879 | −23.025 | −36.660 | 70.150 | 1.00 | 197.24 | C |
| ATOM | 6555 | N | LEU | B | 1880 | −27.219 | −38.645 | 75.690 | 1.00 | 194.41 | N |
| ATOM | 6556 | CA | LEU | B | 1880 | −28.054 | −37.981 | 76.703 | 1.00 | 194.17 | C |
| ATOM | 6557 | C | LEU | B | 1880 | −27.776 | −38.317 | 78.207 | 1.00 | 194.74 | C |
| ATOM | 6558 | O | LEU | B | 1880 | −28.178 | −39.380 | 78.731 | 1.00 | 194.78 | O |
| ATOM | 6559 | CB | LEU | B | 1880 | −29.546 | −38.134 | 76.349 | 1.00 | 193.57 | C |
| ATOM | 6560 | CG | LEU | B | 1880 | −29.993 | −38.216 | 74.890 | 1.00 | 191.44 | C |
| ATOM | 6561 | CD1 | LEU | B | 1880 | −31.471 | −38.001 | 74.789 | 1.00 | 189.39 | C |
| ATOM | 6562 | CD2 | LEU | B | 1880 | −29.300 | −37.194 | 74.073 | 1.00 | 190.03 | C |
| ATOM | 6563 | N | THR | B | 1881 | −27.084 | −37.387 | 78.878 | 1.00 | 194.96 | N |
| ATOM | 6564 | CA | THR | B | 1881 | −26.757 | −37.468 | 80.306 | 1.00 | 194.91 | C |
| ATOM | 6565 | C | THR | B | 1881 | −27.290 | −36.181 | 80.924 | 1.00 | 194.62 | C |
| ATOM | 6566 | O | THR | B | 1881 | −27.791 | −35.315 | 80.209 | 1.00 | 194.31 | O |
| ATOM | 6567 | CB | THR | B | 1881 | −25.206 | −37.547 | 80.557 | 1.00 | 195.10 | C |
| ATOM | 6568 | OG1 | THR | B | 1881 | −24.563 | −38.360 | 79.558 | 1.00 | 196.15 | O |
| ATOM | 6569 | CG2 | THR | B | 1881 | −24.879 | −38.098 | 81.951 | 1.00 | 194.76 | C |
| ATOM | 6570 | N | ILE | B | 1882 | −27.214 | −36.086 | 82.251 | 1.00 | 194.68 | N |
| ATOM | 6571 | CA | ILE | B | 1882 | −27.413 | −34.831 | 82.986 | 1.00 | 194.68 | C |
| ATOM | 6572 | C | ILE | B | 1882 | −26.161 | −34.599 | 83.829 | 1.00 | 194.79 | C |
| ATOM | 6573 | O | ILE | B | 1882 | −26.078 | −35.047 | 84.970 | 1.00 | 194.63 | O |
| ATOM | 6574 | CB | ILE | B | 1882 | −28.667 | −34.847 | 83.904 | 1.00 | 194.57 | C |
| ATOM | 6575 | CG1 | ILE | B | 1882 | −29.770 | −35.746 | 83.342 | 1.00 | 194.63 | C |
| ATOM | 6576 | CG2 | ILE | B | 1882 | −29.194 | −33.444 | 84.088 | 1.00 | 194.27 | C |
| ATOM | 6577 | CD1 | ILE | B | 1882 | −30.923 | −35.987 | 84.294 | 1.00 | 194.60 | C |
| ATOM | 6578 | N | PHE | B | 1883 | −25.185 | −33.912 | 83.247 | 1.00 | 195.09 | N |
| ATOM | 6579 | CA | PHE | B | 1883 | −23.864 | −33.759 | 83.843 | 1.00 | 195.50 | C |
| ATOM | 6580 | C | PHE | B | 1883 | −23.981 | −33.083 | 85.194 | 1.00 | 195.76 | C |
| ATOM | 6581 | O | PHE | B | 1883 | −24.920 | −32.317 | 85.407 | 1.00 | 195.75 | O |
| ATOM | 6582 | CB | PHE | B | 1883 | −22.980 | −32.915 | 82.925 | 1.00 | 195.61 | C |
| ATOM | 6583 | CG | PHE | B | 1883 | −22.460 | −33.648 | 81.722 | 1.00 | 195.92 | C |
| ATOM | 6584 | CD1 | PHE | B | 1883 | −23.298 | −34.454 | 80.948 | 1.00 | 196.46 | C |
| ATOM | 6585 | CD2 | PHE | B | 1883 | −21.131 | −33.508 | 81.342 | 1.00 | 196.20 | C |
| ATOM | 6586 | CE1 | PHE | B | 1883 | −22.814 | −35.130 | 79.823 | 1.00 | 196.55 | C |
| ATOM | 6587 | CE2 | PHE | B | 1883 | −20.636 | −34.174 | 80.218 | 1.00 | 196.53 | C |
| ATOM | 6588 | CZ | PHE | B | 1883 | −21.485 | −34.988 | 79.456 | 1.00 | 196.46 | C |
| ATOM | 6589 | N | ASP | B | 1884 | −23.036 | −33.375 | 86.096 | 1.00 | 196.19 | N |
| ATOM | 6590 | CA | ASP | B | 1884 | −22.970 | −32.750 | 87.434 | 1.00 | 196.61 | C |
| ATOM | 6591 | C | ASP | B | 1884 | −21.542 | −32.610 | 87.960 | 1.00 | 196.93 | C |
| ATOM | 6592 | O | ASP | B | 1884 | −20.729 | −33.513 | 87.796 | 1.00 | 197.11 | O |
| ATOM | 6593 | CB | ASP | B | 1884 | −23.803 | −33.540 | 88.450 | 1.00 | 196.61 | C |
| ATOM | 6594 | CG | ASP | B | 1884 | −24.016 | −32.783 | 89.754 | 1.00 | 196.41 | C |
| ATOM | 6595 | OD1 | ASP | B | 1884 | −25.188 | −32.479 | 90.073 | 1.00 | 196.00 | O |
| ATOM | 6596 | OD2 | ASP | B | 1884 | −23.020 | −32.489 | 90.454 | 1.00 | 196.15 | O |
| ATOM | 6597 | N | GLU | B | 1885 | −21.247 | −31.495 | 88.620 | 1.00 | 197.28 | N |
| ATOM | 6598 | CA | GLU | B | 1885 | −19.917 | −31.297 | 89.188 | 1.00 | 197.66 | C |
| ATOM | 6599 | C | GLU | B | 1885 | −19.900 | −31.307 | 90.712 | 1.00 | 198.08 | C |
| ATOM | 6600 | O | GLU | B | 1885 | −19.673 | −30.285 | 91.357 | 1.00 | 197.98 | O |
| ATOM | 6601 | CB | GLU | B | 1885 | −19.253 | −30.033 | 88.636 | 1.00 | 197.60 | C |
| ATOM | 6602 | CG | GLU | B | 1885 | −18.987 | −30.077 | 87.143 | 1.00 | 197.23 | C |
| ATOM | 6603 | CD | GLU | B | 1885 | −17.535 | −30.225 | 86.785 | 1.00 | 197.12 | C |
| ATOM | 6604 | OE1 | GLU | B | 1885 | −16.813 | −30.983 | 87.467 | 1.00 | 197.02 | O |
| ATOM | 6605 | OE2 | GLU | B | 1885 | −17.122 | −29.579 | 85.799 | 1.00 | 197.02 | O |
| ATOM | 6606 | N | THR | B | 1886 | −20.195 | −32.473 | 91.272 | 1.00 | 198.73 | N |
| ATOM | 6607 | CA | THR | B | 1886 | −19.631 | −32.876 | 92.556 | 1.00 | 199.53 | C |
| ATOM | 6608 | C | THR | B | 1886 | −18.588 | −33.918 | 92.144 | 1.00 | 199.94 | C |
| ATOM | 6609 | O | THR | B | 1886 | −17.983 | −34.604 | 92.983 | 1.00 | 200.17 | O |
| ATOM | 6610 | CB | THR | B | 1886 | −20.684 | −33.488 | 93.529 | 1.00 | 199.59 | C |
| ATOM | 6611 | OG1 | THR | B | 1886 | −21.904 | −32.743 | 93.456 | 1.00 | 200.01 | O |
| ATOM | 6612 | CG2 | THR | B | 1886 | −20.178 | −33.479 | 94.989 | 1.00 | 199.43 | C |
| ATOM | 6613 | N | LYS | B | 1887 | −18.378 | −34.000 | 90.827 | 1.00 | 200.25 | N |
| ATOM | 6614 | CA | LYS | B | 1887 | −17.590 | −35.051 | 90.201 | 1.00 | 200.51 | C |
| ATOM | 6615 | C | LYS | B | 1887 | −16.438 | −34.459 | 89.369 | 1.00 | 200.95 | C |
| ATOM | 6616 | O | LYS | B | 1887 | −16.590 | −33.395 | 88.762 | 1.00 | 200.81 | O |
| ATOM | 6617 | CB | LYS | B | 1887 | −18.504 | −35.940 | 89.342 | 1.00 | 200.22 | C |
| ATOM | 6618 | CG | LYS | B | 1887 | −19.711 | −36.581 | 90.082 | 1.00 | 199.91 | C |
| ATOM | 6619 | CD | LYS | B | 1887 | −20.951 | −35.667 | 90.122 | 1.00 | 198.93 | C |
| ATOM | 6620 | CE | LYS | B | 1887 | −22.243 | −36.425 | 90.429 | 1.00 | 198.69 | C |
| ATOM | 6621 | NZ | LYS | B | 1887 | −22.398 | −36.785 | 91.860 | 1.00 | 197.55 | N |
| ATOM | 6622 | N | SER | B | 1888 | −15.297 | −35.157 | 89.373 | 1.00 | 201.64 | N |
| ATOM | 6623 | CA | SER | B | 1888 | −14.056 | −34.773 | 88.664 | 1.00 | 202.28 | C |
| ATOM | 6624 | C | SER | B | 1888 | −13.517 | −33.377 | 88.940 | 1.00 | 202.76 | C |
| ATOM | 6625 | O | SER | B | 1888 | −14.094 | −32.381 | 88.503 | 1.00 | 202.68 | O |
| ATOM | 6626 | CB | SER | B | 1888 | −14.194 | −34.952 | 87.161 | 1.00 | 202.28 | C |
| ATOM | 6627 | OG | SER | B | 1888 | −15.005 | −33.931 | 86.603 | 1.00 | 202.55 | O |
| ATOM | 6628 | N | TRP | B | 1889 | −12.392 | −33.342 | 89.650 | 1.00 | 203.55 | N |
| ATOM | 6629 | CA | TRP | B | 1889 | −11.613 | −32.128 | 89.980 | 1.00 | 204.36 | C |
| ATOM | 6630 | C | TRP | B | 1889 | −12.308 | −30.743 | 90.040 | 1.00 | 204.68 | C |
| ATOM | 6631 | O | TRP | B | 1889 | −11.618 | −29.722 | 90.081 | 1.00 | 204.86 | O |
| ATOM | 6632 | CB | TRP | B | 1889 | −10.361 | −32.042 | 89.082 | 1.00 | 204.52 | C |
| ATOM | 6633 | CG | TRP | B | 1889 | −10.648 | −31.755 | 87.618 | 1.00 | 204.90 | C |
| ATOM | 6634 | CD1 | TRP | B | 1889 | −11.672 | −31.002 | 87.104 | 1.00 | 204.97 | C |
| ATOM | 6635 | CD2 | TRP | B | 1889 | −9.875 | −32.190 | 86.492 | 1.00 | 205.25 | C |
| ATOM | 6636 | NE1 | TRP | B | 1889 | −11.593 | −30.960 | 85.734 | 1.00 | 204.94 | N |
| ATOM | 6637 | CE2 | TRP | B | 1889 | −10.499 | −31.679 | 85.332 | 1.00 | 205.06 | C |
| ATOM | 6638 | CE3 | TRP | B | 1889 | −8.720 | −32.970 | 86.350 | 1.00 | 205.47 | C |
| ATOM | 6639 | CZ2 | TRP | B | 1889 | −10.005 | −31.922 | 84.048 | 1.00 | 204.91 | C |
| ATOM | 6640 | CZ3 | TRP | B | 1889 | −8.231 | −33.212 | 85.068 | 1.00 | 205.03 | C |
| ATOM | 6641 | CH2 | TRP | B | 1889 | −8.874 | −32.689 | 83.936 | 1.00 | 204.87 | C |
| ATOM | 6642 | N | TYR | B | 1890 | −13.641 | −30.699 | 90.075 | 1.00 | 204.94 | N |
| ATOM | 6643 | CA | TYR | B | 1890 | −14.386 | −29.429 | 89.964 | 1.00 | 205.11 | C |
| ATOM | 6644 | C | TYR | B | 1890 | −13.944 | −28.318 | 90.928 | 1.00 | 205.06 | C |
| ATOM | 6645 | O | TYR | B | 1890 | −14.204 | −27.139 | 90.676 | 1.00 | 205.03 | O |
| ATOM | 6646 | CB | TYR | B | 1890 | −15.898 | −29.665 | 90.061 | 1.00 | 205.38 | C |
| ATOM | 6647 | CG | TYR | B | 1890 | −16.413 | −29.835 | 91.467 | 1.00 | 205.82 | C |
| ATOM | 6648 | CD1 | TYR | B | 1890 | −16.165 | −31.007 | 92.181 | 1.00 | 206.22 | C |
| ATOM | 6649 | CD2 | TYR | B | 1890 | −17.151 | −28.824 | 92.087 | 1.00 | 205.84 | C |
| ATOM | 6650 | CE1 | TYR | B | 1890 | −16.631 | −31.170 | 93.477 | 1.00 | 206.35 | C |
| ATOM | 6651 | CE2 | TYR | B | 1890 | −17.627 | −28.978 | 93.384 | 1.00 | 205.97 | C |
| ATOM | 6652 | CZ | TYR | B | 1890 | −17.364 | −30.156 | 94.073 | 1.00 | 206.20 | C |
| ATOM | 6653 | OH | TYR | B | 1890 | −17.826 | −30.330 | 95.361 | 1.00 | 206.36 | O |
| ATOM | 6654 | N | PHE | B | 1891 | −13.289 | −28.706 | 92.025 | 1.00 | 204.98 | N |
| ATOM | 6655 | CA | PHE | B | 1891 | −12.623 | −27.769 | 92.931 | 1.00 | 204.93 | C |
| ATOM | 6656 | C | PHE | B | 1891 | −11.830 | −26.710 | 92.148 | 1.00 | 205.04 | C |
| ATOM | 6657 | O | PHE | B | 1891 | −11.966 | −25.510 | 92.405 | 1.00 | 205.01 | O |
| ATOM | 6658 | CB | PHE | B | 1891 | −11.726 | −28.531 | 93.923 | 1.00 | 204.78 | C |
| ATOM | 6659 | CG | PHE | B | 1891 | −12.316 | −28.673 | 95.316 | 1.00 | 204.72 | C |
| ATOM | 6660 | CD1 | PHE | B | 1891 | −13.678 | −28.911 | 95.507 | 1.00 | 204.50 | C |
| ATOM | 6661 | CD2 | PHE | B | 1891 | −11.492 | −28.586 | 96.443 | 1.00 | 204.73 | C |
| ATOM | 6662 | CE1 | PHE | B | 1891 | −14.214 | −29.041 | 96.803 | 1.00 | 204.37 | C |
| ATOM | 6663 | CE2 | PHE | B | 1891 | −12.017 | −28.719 | 97.742 | 1.00 | 204.47 | C |
| ATOM | 6664 | CZ | PHE | B | 1891 | −13.379 | −28.947 | 97.919 | 1.00 | 204.43 | C |
| ATOM | 6665 | N | THR | B | 1892 | −11.038 | −27.156 | 91.170 | 1.00 | 205.20 | N |
| ATOM | 6666 | CA | THR | B | 1892 | −10.340 | −26.249 | 90.244 | 1.00 | 205.34 | C |
| ATOM | 6667 | C | THR | B | 1892 | −11.304 | −25.556 | 89.260 | 1.00 | 205.47 | C |
| ATOM | 6668 | O | THR | B | 1892 | −10.996 | −25.385 | 88.079 | 1.00 | 205.46 | O |
| ATOM | 6669 | CB | THR | B | 1892 | −9.137 | −26.947 | 89.498 | 1.00 | 205.32 | C |
| ATOM | 6670 | OG1 | THR | B | 1892 | −8.469 | −26.003 | 88.653 | 1.00 | 205.32 | O |
| ATOM | 6671 | CG2 | THR | B | 1892 | −9.584 | −28.132 | 88.643 | 1.00 | 205.24 | C |
| ATOM | 6672 | N | GLU | B | 1893 | −12.472 | −25.166 | 89.765 | 1.00 | 205.62 | N |
| ATOM | 6673 | CA | GLU | B | 1893 | −13.450 | −24.414 | 88.993 | 1.00 | 205.86 | C |
| ATOM | 6674 | C | GLU | B | 1893 | −14.211 | −23.468 | 89.897 | 1.00 | 206.05 | C |
| ATOM | 6675 | O | GLU | B | 1893 | −14.434 | −22.319 | 89.539 | 1.00 | 206.08 | O |
| ATOM | 6676 | CB | GLU | B | 1893 | −14.422 | −25.347 | 88.287 | 1.00 | 205.78 | C |
| ATOM | 6677 | CG | GLU | B | 1893 | −13.874 | −25.952 | 87.012 | 1.00 | 206.42 | C |
| ATOM | 6678 | CD | GLU | B | 1893 | −14.601 | −27.220 | 86.604 | 1.00 | 207.42 | C |
| ATOM | 6679 | OE1 | GLU | B | 1893 | −14.850 | −28.065 | 87.488 | 1.00 | 207.65 | O |
| ATOM | 6680 | OE2 | GLU | B | 1893 | −14.919 | −27.379 | 85.402 | 1.00 | 207.68 | O |
| ATOM | 6681 | N | ASN | B | 1894 | −14.593 | −23.954 | 91.073 | 1.00 | 206.37 | N |
| ATOM | 6682 | CA | ASN | B | 1894 | −15.357 | −23.160 | 92.033 | 1.00 | 206.79 | C |
| ATOM | 6683 | C | ASN | B | 1894 | −14.601 | −21.939 | 92.539 | 1.00 | 207.17 | C |
| ATOM | 6684 | O | ASN | B | 1894 | −15.162 | −20.846 | 92.640 | 1.00 | 207.21 | O |
| ATOM | 6685 | CB | ASN | B | 1894 | −15.784 | −24.024 | 93.216 | 1.00 | 206.71 | C |
| ATOM | 6686 | CG | ASN | B | 1894 | −16.581 | −25.233 | 92.792 | 1.00 | 206.65 | C |
| ATOM | 6687 | OD1 | ASN | B | 1894 | −16.531 | −25.653 | 91.636 | 1.00 | 206.67 | O |
| ATOM | 6688 | ND2 | ASN | B | 1894 | −17.322 | −25.805 | 93.731 | 1.00 | 206.50 | N |
| ATOM | 6689 | N | MET | B | 1895 | −13.329 | −22.131 | 92.866 | 1.00 | 207.63 | N |
| ATOM | 6690 | CA | MET | B | 1895 | −12.491 | −21.023 | 93.295 | 1.00 | 208.19 | C |
| ATOM | 6691 | C | MET | B | 1895 | −11.391 | −20.717 | 92.275 | 1.00 | 208.48 | C |
| ATOM | 6692 | O | MET | B | 1895 | −10.457 | −19.963 | 92.560 | 1.00 | 208.52 | O |
| ATOM | 6693 | CB | MET | B | 1895 | −11.938 | −21.264 | 94.708 | 1.00 | 208.17 | C |
| ATOM | 6694 | CG | MET | B | 1895 | −12.855 | −20.726 | 95.822 | 1.00 | 208.35 | C |
| ATOM | 6695 | SD | MET | B | 1895 | −12.205 | −20.882 | 97.508 | 1.00 | 208.48 | S |
| ATOM | 6696 | CE | MET | B | 1895 | −12.999 | −19.511 | 98.366 | 1.00 | 208.27 | C |
| ATOM | 6697 | N | GLU | B | 1896 | −11.521 | −21.303 | 91.085 | 1.00 | 208.92 | N |
| ATOM | 6698 | CA | GLU | B | 1896 | −10.684 | −20.947 | 89.936 | 1.00 | 209.37 | C |
| ATOM | 6699 | C | GLU | B | 1896 | −11.329 | −19.785 | 89.185 | 1.00 | 209.45 | C |
| ATOM | 6700 | O | GLU | B | 1896 | −10.660 | −18.824 | 88.793 | 1.00 | 209.51 | O |
| ATOM | 6701 | CB | GLU | B | 1896 | −10.516 | −22.143 | 88.992 | 1.00 | 209.48 | C |
| ATOM | 6702 | CG | GLU | B | 1896 | −9.695 | −21.846 | 87.728 | 1.00 | 210.02 | C |
| ATOM | 6703 | CD | GLU | B | 1896 | −10.186 | −22.597 | 86.493 | 1.00 | 210.67 | C |
| ATOM | 6704 | OE1 | GLU | B | 1896 | −11.374 | −22.993 | 86.444 | 1.00 | 210.92 | O |
| ATOM | 6705 | OE2 | GLU | B | 1896 | −9.376 | −22.778 | 85.558 | 1.00 | 210.89 | O |
| ATOM | 6706 | N | ARG | B | 1897 | −12.635 | −19.899 | 88.983 | 1.00 | 209.54 | N |
| ATOM | 6707 | CA | ARG | B | 1897 | −13.422 | −18.876 | 88.331 | 1.00 | 209.70 | C |
| ATOM | 6708 | C | ARG | B | 1897 | −14.684 | −18.692 | 89.179 | 1.00 | 209.98 | C |
| ATOM | 6709 | O | ARG | B | 1897 | −14.997 | −19.547 | 90.006 | 1.00 | 209.97 | O |
| ATOM | 6710 | CB | ARG | B | 1897 | −13.684 | −19.297 | 86.875 | 1.00 | 209.56 | C |
| ATOM | 6711 | CG | ARG | B | 1897 | −14.827 | −18.607 | 86.146 | 1.00 | 209.35 | C |
| ATOM | 6712 | CD | ARG | B | 1897 | −15.997 | −19.560 | 85.887 | 1.00 | 208.58 | C |
| ATOM | 6713 | NE | ARG | B | 1897 | −16.637 | −20.031 | 87.114 | 1.00 | 207.95 | N |
| ATOM | 6714 | CZ | ARG | B | 1897 | −16.335 | −21.167 | 87.736 | 1.00 | 207.54 | C |
| ATOM | 6715 | NH1 | ARG | B | 1897 | −15.389 | −21.966 | 87.260 | 1.00 | 207.31 | N |
| ATOM | 6716 | NH2 | ARG | B | 1897 | −16.970 | −21.500 | 88.850 | 1.00 | 207.40 | N |
| ATOM | 6717 | N | ASN | B | 1898 | −15.372 | −17.564 | 89.000 | 1.00 | 210.39 | N |
| ATOM | 6718 | CA | ASN | B | 1898 | −16.559 | −17.192 | 89.794 | 1.00 | 210.77 | C |
| ATOM | 6719 | C | ASN | B | 1898 | −16.267 | −16.593 | 91.170 | 1.00 | 211.30 | C |
| ATOM | 6720 | O | ASN | B | 1898 | −17.186 | −16.147 | 91.863 | 1.00 | 211.31 | O |
| ATOM | 6721 | CB | ASN | B | 1898 | −17.547 | −18.359 | 89.933 | 1.00 | 210.55 | C |
| ATOM | 6722 | CG | ASN | B | 1898 | −18.704 | −18.269 | 88.963 | 1.00 | 210.03 | C |
| ATOM | 6723 | OD1 | ASN | B | 1898 | −19.734 | −18.908 | 89.160 | 1.00 | 209.28 | O |
| ATOM | 6724 | ND2 | ASN | B | 1898 | −18.545 | −17.471 | 87.913 | 1.00 | 209.60 | N |
| ATOM | 6725 | N | CYS | B | 1899 | −14.994 | −16.567 | 91.556 | 1.00 | 211.99 | N |
| ATOM | 6726 | CA | CYS | B | 1899 | −14.625 | −16.104 | 92.889 | 1.00 | 212.77 | C |
| ATOM | 6727 | C | CYS | B | 1899 | −13.257 | −15.429 | 92.984 | 1.00 | 212.93 | C |
| ATOM | 6728 | O | CYS | B | 1899 | −12.270 | −15.907 | 92.418 | 1.00 | 212.92 | O |
| ATOM | 6729 | CB | CYS | B | 1899 | −14.712 | −17.263 | 93.885 | 1.00 | 212.97 | C |
| ATOM | 6730 | SG | CYS | B | 1899 | −16.282 | −17.359 | 94.786 | 1.00 | 214.24 | S |
| ATOM | 6731 | N | ARG | B | 1900 | −13.229 | −14.304 | 93.701 | 1.00 | 213.25 | N |
| ATOM | 6732 | CA | ARG | B | 1900 | −12.002 | −13.571 | 94.029 | 1.00 | 213.61 | C |
| ATOM | 6733 | C | ARG | B | 1900 | −12.148 | −12.993 | 95.447 | 1.00 | 213.94 | C |
| ATOM | 6734 | O | ARG | B | 1900 | −12.163 | −13.745 | 96.429 | 1.00 | 213.99 | O |
| ATOM | 6735 | CB | ARG | B | 1900 | −11.723 | −12.442 | 93.017 | 1.00 | 213.55 | C |
| ATOM | 6736 | CG | ARG | B | 1900 | −11.635 | −12.852 | 91.550 | 1.00 | 213.36 | C |
| ATOM | 6737 | CD | ARG | B | 1900 | −12.997 | −12.814 | 90.875 | 1.00 | 213.00 | C |
| ATOM | 6738 | NE | ARG | B | 1900 | −13.051 | −13.706 | 89.721 | 1.00 | 212.83 | N |
| ATOM | 6739 | CZ | ARG | B | 1900 | −14.172 | −14.191 | 89.196 | 1.00 | 212.66 | C |
| ATOM | 6740 | NH1 | ARG | B | 1900 | −15.349 | −13.879 | 89.724 | 1.00 | 212.70 | N |
| ATOM | 6741 | NH2 | ARG | B | 1900 | −14.115 | −14.994 | 88.141 | 1.00 | 212.37 | N |
| ATOM | 6742 | N | ALA | B | 1901 | −12.254 | −11.662 | 95.535 | 1.00 | 214.32 | N |
| ATOM | 6743 | CA | ALA | B | 1901 | −12.630 | −10.948 | 96.769 | 1.00 | 214.63 | C |
| ATOM | 6744 | C | ALA | B | 1901 | −14.160 | −10.771 | 96.951 | 1.00 | 214.90 | C |
| ATOM | 6745 | O | ALA | B | 1901 | −14.598 | −10.434 | 98.060 | 1.00 | 214.89 | O |
| ATOM | 6746 | CB | ALA | B | 1901 | −11.907 | −9.595 | 96.864 | 1.00 | 214.49 | C |
| ATOM | 6747 | N | PRO | B | 1902 | −14.967 | −10.947 | 95.862 | 1.00 | 215.15 | N |
| ATOM | 6748 | CA | PRO | B | 1902 | −16.414 | −11.190 | 95.978 | 1.00 | 215.26 | C |
| ATOM | 6749 | C | PRO | B | 1902 | −16.792 | −12.686 | 95.930 | 1.00 | 215.34 | C |
| ATOM | 6750 | O | PRO | B | 1902 | −17.277 | −13.186 | 94.903 | 1.00 | 215.33 | O |
| ATOM | 6751 | CB | PRO | B | 1902 | −16.981 | −10.457 | 94.758 | 1.00 | 215.26 | C |
| ATOM | 6752 | CG | PRO | B | 1902 | −15.885 | −10.530 | 93.724 | 1.00 | 215.22 | C |
| ATOM | 6753 | CD | PRO | B | 1902 | −14.579 | −10.830 | 94.440 | 1.00 | 215.20 | C |
| ATOM | 6754 | N | CYS | B | 1903 | −16.580 | −13.377 | 97.048 | 1.00 | 215.46 | N |
| ATOM | 6755 | CA | CYS | B | 1903 | −16.840 | −14.812 | 97.145 | 1.00 | 215.51 | C |
| ATOM | 6756 | C | CYS | B | 1903 | −17.688 | −15.165 | 98.380 | 1.00 | 215.58 | C |
| ATOM | 6757 | O | CYS | B | 1903 | −18.503 | −14.350 | 98.825 | 1.00 | 215.70 | O |
| ATOM | 6758 | CB | CYS | B | 1903 | −15.517 | −15.582 | 97.138 | 1.00 | 215.42 | C |
| ATOM | 6759 | SG | CYS | B | 1903 | −15.703 | −17.331 | 96.747 | 1.00 | 215.56 | S |
| ATOM | 6760 | N | ASN | B | 1904 | −17.492 | −16.374 | 98.917 | 1.00 | 215.57 | N |
| ATOM | 6761 | CA | ASN | B | 1904 | −18.255 | −16.899 | 100.066 | 1.00 | 215.51 | C |
| ATOM | 6762 | C | ASN | B | 1904 | −19.783 | −16.740 | 99.927 | 1.00 | 215.48 | C |
| ATOM | 6763 | O | ASN | B | 1904 | −20.460 | −16.206 | 100.817 | 1.00 | 215.51 | O |
| ATOM | 6764 | CB | ASN | B | 1904 | −17.735 | −16.316 | 101.394 | 1.00 | 215.47 | C |
| ATOM | 6765 | CG | ASN | B | 1904 | −16.276 | −16.697 | 101.675 | 1.00 | 215.35 | C |
| ATOM | 6766 | OD1 | ASN | B | 1904 | −15.374 | −17.019 | 100.588 | 1.00 | 214.94 | O |
| ATOM | 6767 | ND2 | ASN | B | 1904 | −16.045 | −16.624 | 103.188 | 1.00 | 215.37 | N |
| ATOM | 6768 | N | ILE | B | 1905 | −20.302 | −17.211 | 98.793 | 1.00 | 215.36 | N |
| ATOM | 6769 | CA | ILE | B | 1905 | −21.732 | −17.171 | 98.485 | 1.00 | 215.19 | C |
| ATOM | 6770 | C | ILE | B | 1905 | −22.207 | −18.539 | 97.969 | 1.00 | 215.17 | C |
| ATOM | 6771 | O | ILE | B | 1905 | −21.800 | −18.979 | 96.888 | 1.00 | 215.16 | O |
| ATOM | 6772 | CB | ILE | B | 1905 | −22.094 | −16.013 | 97.488 | 1.00 | 215.21 | C |
| ATOM | 6773 | CG1 | ILE | B | 1905 | −20.995 | −15.820 | 96.422 | 1.00 | 215.15 | C |
| ATOM | 6774 | CG2 | ILE | B | 1905 | −22.354 | −14.715 | 98.255 | 1.00 | 214.96 | C |
| ATOM | 6775 | CD1 | ILE | B | 1905 | −21.372 | −14.916 | 95.239 | 1.00 | 215.05 | C |
| ATOM | 6776 | N | GLN | B | 1906 | −23.054 | −19.200 | 98.767 | 1.00 | 215.08 | N |
| ATOM | 6777 | CA | GLN | B | 1906 | −23.610 | −20.550 | 98.483 | 1.00 | 214.99 | C |
| ATOM | 6778 | C | GLN | B | 1906 | −22.563 | −21.682 | 98.434 | 1.00 | 214.75 | C |
| ATOM | 6779 | O | GLN | B | 1906 | −21.886 | −21.868 | 97.419 | 1.00 | 214.78 | O |
| ATOM | 6780 | CB | GLN | B | 1906 | −24.490 | −20.568 | 97.210 | 1.00 | 215.07 | C |
| ATOM | 6781 | CG | GLN | B | 1906 | −25.783 | −19.731 | 97.270 | 1.00 | 215.31 | C |
| ATOM | 6782 | CD | GLN | B | 1906 | −26.955 | −20.442 | 97.951 | 1.00 | 215.44 | C |
| ATOM | 6783 | OE1 | GLN | B | 1906 | −26.943 | −20.678 | 99.161 | 1.00 | 215.66 | O |
| ATOM | 6784 | NE2 | GLN | B | 1906 | −27.984 | −20.761 | 97.170 | 1.00 | 215.15 | N |
| ATOM | 6785 | N | MET | B | 1907 | −22.455 | −22.439 | 99.529 | 1.00 | 214.41 | N |
| ATOM | 6786 | CA | MET | B | 1907 | −21.515 | −23.568 | 99.625 | 1.00 | 214.06 | C |
| ATOM | 6787 | C | MET | B | 1907 | −21.990 | −24.829 | 98.862 | 1.00 | 213.90 | C |
| ATOM | 6788 | O | MET | B | 1907 | −21.308 | −25.306 | 97.945 | 1.00 | 213.82 | O |
| ATOM | 6789 | CB | MET | B | 1907 | −21.153 | −23.879 | 101.095 | 1.00 | 213.96 | C |
| ATOM | 6790 | CG | MET | B | 1907 | −22.334 | −24.021 | 102.074 | 1.00 | 213.88 | C |
| ATOM | 6791 | SD | MET | B | 1907 | −22.728 | −22.543 | 103.038 | 1.00 | 213.89 | S |
| ATOM | 6792 | CE | MET | B | 1907 | −24.202 | −23.075 | 103.910 | 1.00 | 213.65 | C |
| ATOM | 6793 | N | GLU | B | 1908 | −23.147 | −25.363 | 99.258 | 1.00 | 213.67 | N |
| ATOM | 6794 | CA | GLU | B | 1908 | −23.818 | −26.463 | 98.546 | 1.00 | 213.38 | C |
| ATOM | 6795 | C | GLU | B | 1908 | −25.350 | −26.326 | 98.671 | 1.00 | 213.09 | C |
| ATOM | 6796 | O | GLU | B | 1908 | −26.095 | −27.197 | 98.211 | 1.00 | 213.09 | O |
| ATOM | 6797 | CB | GLU | B | 1908 | −23.363 | −27.854 | 99.037 | 1.00 | 213.41 | C |
| ATOM | 6798 | CG | GLU | B | 1908 | −22.104 | −27.902 | 99.919 | 1.00 | 213.79 | C |
| ATOM | 6799 | CD | GLU | B | 1908 | −22.398 | −27.685 | 101.413 | 1.00 | 214.44 | C |
| ATOM | 6800 | OE1 | GLU | B | 1908 | −23.473 | −28.106 | 101.892 | 1.00 | 214.65 | O |
| ATOM | 6801 | OE2 | GLU | B | 1908 | −21.544 | −27.104 | 102.121 | 1.00 | 214.62 | O |
| ATOM | 6802 | N | ASP | B | 1909 | −25.795 | −25.235 | 99.308 | 1.00 | 212.66 | N |
| ATOM | 6803 | CA | ASP | B | 1909 | −27.217 | −24.850 | 99.430 | 1.00 | 212.21 | C |
| ATOM | 6804 | C | ASP | B | 1909 | −27.705 | −24.176 | 98.140 | 1.00 | 211.77 | C |
| ATOM | 6805 | O | ASP | B | 1909 | −26.925 | −23.466 | 97.507 | 1.00 | 211.87 | O |
| ATOM | 6806 | CB | ASP | B | 1909 | −27.406 | −23.893 | 100.618 | 1.00 | 212.33 | C |
| ATOM | 6807 | CG | ASP | B | 1909 | −27.788 | −24.613 | 101.904 | 1.00 | 212.59 | C |
| ATOM | 6808 | OD1 | ASP | B | 1909 | −28.457 | −25.669 | 101.817 | 1.00 | 212.91 | O |
| ATOM | 6809 | OD2 | ASP | B | 1909 | −27.435 | −24.111 | 103.001 | 1.00 | 212.52 | O |
| ATOM | 6810 | N | PRO | B | 1910 | −29.011 | −24.315 | 97.795 | 1.00 | 211.21 | N |
| ATOM | 6811 | CA | PRO | B | 1910 | −29.618 | −24.210 | 96.455 | 1.00 | 210.66 | C |
| ATOM | 6812 | C | PRO | B | 1910 | −28.742 | −23.763 | 95.256 | 1.00 | 210.09 | C |
| ATOM | 6813 | O | PRO | B | 1910 | −29.234 | −23.090 | 94.340 | 1.00 | 209.89 | O |
| ATOM | 6814 | CB | PRO | B | 1910 | −30.799 | −23.263 | 96.708 | 1.00 | 210.72 | C |
| ATOM | 6815 | CG | PRO | B | 1910 | −31.150 | −23.498 | 98.225 | 1.00 | 210.90 | C |
| ATOM | 6816 | CD | PRO | B | 1910 | −30.087 | −24.435 | 98.793 | 1.00 | 211.14 | C |
| ATOM | 6817 | N | THR | B | 1911 | −27.475 | −24.181 | 95.255 | 1.00 | 209.49 | N |
| ATOM | 6818 | CA | THR | B | 1911 | −26.484 | −23.739 | 94.269 | 1.00 | 208.83 | C |
| ATOM | 6819 | C | THR | B | 1911 | −26.454 | −24.633 | 93.038 | 1.00 | 208.39 | C |
| ATOM | 6820 | O | THR | B | 1911 | −26.308 | −24.129 | 91.928 | 1.00 | 208.50 | O |
| ATOM | 6821 | CB | THR | B | 1911 | −25.047 | −23.592 | 94.887 | 1.00 | 208.86 | C |
| ATOM | 6822 | OG1 | THR | B | 1911 | −24.246 | −22.739 | 94.062 | 1.00 | 208.74 | O |
| ATOM | 6823 | CG2 | THR | B | 1911 | −24.341 | −24.949 | 95.063 | 1.00 | 208.69 | C |
| ATOM | 6824 | N | PHE | B | 1912 | −26.583 | −25.947 | 93.254 | 1.00 | 207.69 | N |
| ATOM | 6825 | CA | PHE | B | 1912 | −26.641 | −26.969 | 92.190 | 1.00 | 206.96 | C |
| ATOM | 6826 | C | PHE | B | 1912 | −26.225 | −26.475 | 90.788 | 1.00 | 206.29 | C |
| ATOM | 6827 | O | PHE | B | 1912 | −27.078 | −26.139 | 89.981 | 1.00 | 206.12 | O |
| ATOM | 6828 | CB | PHE | B | 1912 | −28.036 | −27.644 | 92.159 | 1.00 | 207.11 | C |
| ATOM | 6829 | CG | PHE | B | 1912 | −29.190 | −26.738 | 92.582 | 1.00 | 207.58 | C |
| ATOM | 6830 | CD1 | PHE | B | 1912 | −29.670 | −25.734 | 91.738 | 1.00 | 208.00 | C |
| ATOM | 6831 | CD2 | PHE | B | 1912 | −29.810 | −26.909 | 93.822 | 1.00 | 207.92 | C |
| ATOM | 6832 | CE1 | PHE | B | 1912 | −30.737 | −24.902 | 92.127 | 1.00 | 207.97 | C |
| ATOM | 6833 | CE2 | PHE | B | 1912 | −30.883 | −26.086 | 94.216 | 1.00 | 207.79 | C |
| ATOM | 6834 | CZ | PHE | B | 1912 | −31.342 | −25.081 | 93.367 | 1.00 | 207.62 | C |
| ATOM | 6835 | N | LYS | B | 1913 | −24.922 | −26.440 | 90.504 | 1.00 | 205.44 | N |
| ATOM | 6836 | CA | LYS | B | 1913 | −24.408 | −25.748 | 89.310 | 1.00 | 204.66 | C |
| ATOM | 6837 | C | LYS | B | 1913 | −24.597 | −26.451 | 87.966 | 1.00 | 204.47 | C |
| ATOM | 6838 | O | LYS | B | 1913 | −24.387 | −25.835 | 86.920 | 1.00 | 204.57 | O |
| ATOM | 6839 | CB | LYS | B | 1913 | −22.946 | −25.350 | 89.492 | 1.00 | 204.46 | C |
| ATOM | 6840 | CG | LYS | B | 1913 | −22.014 | −26.508 | 89.715 | 1.00 | 203.98 | C |
| ATOM | 6841 | CD | LYS | B | 1913 | −20.849 | −26.076 | 90.558 | 1.00 | 204.15 | C |
| ATOM | 6842 | CE | LYS | B | 1913 | −21.323 | −25.635 | 91.934 | 1.00 | 204.69 | C |
| ATOM | 6843 | NZ | LYS | B | 1913 | −20.247 | −24.984 | 92.726 | 1.00 | 205.35 | N |
| ATOM | 6844 | N | GLU | B | 1914 | −24.979 | −27.730 | 87.998 | 1.00 | 204.14 | N |
| ATOM | 6845 | CA | GLU | B | 1914 | −25.311 | −28.514 | 86.787 | 1.00 | 203.66 | C |
| ATOM | 6846 | C | GLU | B | 1914 | −26.772 | −28.950 | 86.768 | 1.00 | 203.31 | C |
| ATOM | 6847 | O | GLU | B | 1914 | −27.342 | −29.186 | 85.706 | 1.00 | 203.00 | O |
| ATOM | 6848 | CB | GLU | B | 1914 | −24.425 | −29.743 | 86.670 | 1.00 | 203.67 | C |
| ATOM | 6849 | CG | GLU | B | 1914 | −23.216 | −29.558 | 85.784 | 1.00 | 203.83 | C |
| ATOM | 6850 | CD | GLU | B | 1914 | −21.961 | −29.194 | 86.535 | 1.00 | 203.81 | C |
| ATOM | 6851 | OE1 | GLU | B | 1914 | −22.011 | −29.078 | 87.777 | 1.00 | 203.94 | O |
| ATOM | 6852 | OE2 | GLU | B | 1914 | −20.912 | −29.035 | 85.875 | 1.00 | 203.70 | O |
| ATOM | 6853 | N | ASN | B | 1915 | −27.340 | −29.108 | 87.964 | 1.00 | 203.15 | N |
| ATOM | 6854 | CA | ASN | B | 1915 | −28.783 | −28.992 | 88.186 | 1.00 | 202.96 | C |
| ATOM | 6855 | C | ASN | B | 1915 | −29.129 | −27.485 | 88.386 | 1.00 | 203.37 | C |
| ATOM | 6856 | O | ASN | B | 1915 | −30.098 | −27.113 | 89.070 | 1.00 | 203.41 | O |
| ATOM | 6857 | CB | ASN | B | 1915 | −29.238 | −29.875 | 89.364 | 1.00 | 202.54 | C |
| ATOM | 6858 | CG | ASN | B | 1915 | −29.359 | −31.350 | 88.990 | 1.00 | 200.79 | C |
| ATOM | 6859 | OD1 | ASN | B | 1915 | −28.452 | −32.143 | 89.234 | 1.00 | 198.56 | O |
| ATOM | 6860 | ND2 | ASN | B | 1915 | −30.489 | −31.720 | 88.404 | 1.00 | 198.89 | N |
| ATOM | 6861 | N | TYR | B | 1916 | −28.268 | −26.644 | 87.802 | 1.00 | 203.63 | N |
| ATOM | 6862 | CA | TYR | B | 1916 | −28.497 | −25.223 | 87.519 | 1.00 | 203.80 | C |
| ATOM | 6863 | C | TYR | B | 1916 | −28.549 | −25.111 | 85.996 | 1.00 | 204.10 | C |
| ATOM | 6864 | O | TYR | B | 1916 | −29.030 | −24.111 | 85.442 | 1.00 | 204.27 | O |
| ATOM | 6865 | CB | TYR | B | 1916 | −27.299 | −24.408 | 87.995 | 1.00 | 203.59 | C |
| ATOM | 6866 | CG | TYR | B | 1916 | −27.632 | −23.112 | 88.666 | 1.00 | 203.39 | C |
| ATOM | 6867 | CD1 | TYR | B | 1916 | −28.062 | −23.096 | 89.990 | 1.00 | 203.22 | C |
| ATOM | 6868 | CD2 | TYR | B | 1916 | −27.495 | −21.902 | 87.995 | 1.00 | 202.98 | C |
| ATOM | 6869 | CE1 | TYR | B | 1916 | −28.359 | −21.922 | 90.631 | 1.00 | 202.90 | C |
| ATOM | 6870 | CE2 | TYR | B | 1916 | −27.795 | −20.713 | 88.624 | 1.00 | 203.03 | C |
| ATOM | 6871 | CZ | TYR | B | 1916 | −28.227 | −20.734 | 89.946 | 1.00 | 203.27 | C |
| ATOM | 6872 | OH | TYR | B | 1916 | −28.532 | −19.567 | 90.600 | 1.00 | 203.74 | O |
| ATOM | 6873 | N | ARG | B | 1917 | −28.028 | −26.155 | 85.339 | 1.00 | 204.21 | N |
| ATOM | 6874 | CA | ARG | B | 1917 | −27.817 | −26.201 | 83.899 | 1.00 | 204.10 | C |
| ATOM | 6875 | C | ARG | B | 1917 | −27.943 | −27.619 | 83.366 | 1.00 | 203.84 | C |
| ATOM | 6876 | O | ARG | B | 1917 | −29.043 | −28.077 | 83.044 | 1.00 | 203.79 | O |
| ATOM | 6877 | CB | ARG | B | 1917 | −26.412 | −25.695 | 83.552 | 1.00 | 204.24 | C |
| ATOM | 6878 | CG | ARG | B | 1917 | −26.203 | −24.200 | 83.667 | 1.00 | 205.12 | C |
| ATOM | 6879 | CD | ARG | B | 1917 | −25.031 | −23.759 | 82.800 | 1.00 | 206.71 | C |
| ATOM | 6880 | NE | ARG | B | 1917 | −25.037 | −22.313 | 82.573 | 1.00 | 207.61 | N |
| ATOM | 6881 | CZ | ARG | B | 1917 | −24.502 | −21.701 | 81.516 | 1.00 | 207.90 | C |
| ATOM | 6882 | NH1 | ARG | B | 1917 | −23.910 | −22.397 | 80.548 | 1.00 | 207.74 | N |
| ATOM | 6883 | NH2 | ARG | B | 1917 | −24.572 | −20.378 | 81.423 | 1.00 | 208.33 | N |
| ATOM | 6884 | N | PHE | B | 1918 | −26.798 | −28.308 | 83.367 | 1.00 | 203.58 | N |
| ATOM | 6885 | CA | PHE | B | 1918 | −26.451 | −29.423 | 82.458 | 1.00 | 203.37 | C |
| ATOM | 6886 | C | PHE | B | 1918 | −27.438 | −30.577 | 82.240 | 1.00 | 203.27 | C |
| ATOM | 6887 | O | PHE | B | 1918 | −27.567 | −31.471 | 83.080 | 1.00 | 203.07 | O |
| ATOM | 6888 | CB | PHE | B | 1918 | −25.066 | −29.976 | 82.822 | 1.00 | 203.12 | C |
| ATOM | 6889 | CG | PHE | B | 1918 | −23.920 | −29.033 | 82.510 | 1.00 | 202.67 | C |
| ATOM | 6890 | CD1 | PHE | B | 1918 | −22.910 | −29.419 | 81.632 | 1.00 | 201.79 | C |
| ATOM | 6891 | CD2 | PHE | B | 1918 | −23.842 | −27.773 | 83.107 | 1.00 | 201.66 | C |
| ATOM | 6892 | CE1 | PHE | B | 1918 | −21.854 | −28.571 | 81.354 | 1.00 | 200.87 | C |
| ATOM | 6893 | CE2 | PHE | B | 1918 | −22.788 | −26.923 | 82.828 | 1.00 | 201.19 | C |
| ATOM | 6894 | CZ | PHE | B | 1918 | −21.791 | −27.327 | 81.947 | 1.00 | 201.42 | C |
| ATOM | 6895 | N | HIS | B | 1919 | −28.086 | −30.549 | 81.073 | 1.00 | 203.23 | N |
| ATOM | 6896 | CA | HIS | B | 1919 | −28.990 | −31.600 | 80.598 | 1.00 | 203.18 | C |
| ATOM | 6897 | C | HIS | B | 1919 | −28.566 | −32.054 | 79.199 | 1.00 | 203.30 | C |
| ATOM | 6898 | O | HIS | B | 1919 | −29.290 | −31.875 | 78.215 | 1.00 | 202.93 | O |
| ATOM | 6899 | CB | HIS | B | 1919 | −30.427 | −31.096 | 80.552 | 1.00 | 203.15 | C |
| ATOM | 6900 | CG | HIS | B | 1919 | −31.126 | −31.114 | 81.873 | 1.00 | 202.48 | C |
| ATOM | 6901 | ND1 | HIS | B | 1919 | −31.168 | −30.018 | 82.705 | 1.00 | 202.06 | N |
| ATOM | 6902 | CD2 | HIS | B | 1919 | −31.839 | −32.085 | 82.488 | 1.00 | 201.92 | C |
| ATOM | 6903 | CE1 | HIS | B | 1919 | −31.866 | −30.318 | 83.784 | 1.00 | 202.25 | C |
| ATOM | 6904 | NE2 | HIS | B | 1919 | −32.284 | −31.567 | 83.677 | 1.00 | 201.93 | N |
| ATOM | 6905 | N | ALA | B | 1920 | −27.394 | −32.675 | 79.150 | 1.00 | 203.68 | N |
| ATOM | 6906 | CA | ALA | B | 1920 | −26.687 | −32.999 | 77.914 | 1.00 | 204.07 | C |
| ATOM | 6907 | C | ALA | B | 1920 | −27.447 | −33.760 | 76.812 | 1.00 | 204.23 | C |
| ATOM | 6908 | O | ALA | B | 1920 | −28.291 | −34.624 | 77.092 | 1.00 | 204.55 | O |
| ATOM | 6909 | CB | ALA | B | 1920 | −25.381 | −33.716 | 78.242 | 1.00 | 204.12 | C |
| ATOM | 6910 | N | ILE | B | 1921 | −27.132 | −33.379 | 75.569 | 1.00 | 204.01 | N |
| ATOM | 6911 | CA | ILE | B | 1921 | −27.330 | −34.148 | 74.339 | 1.00 | 203.62 | C |
| ATOM | 6912 | C | ILE | B | 1921 | −25.941 | −34.106 | 73.707 | 1.00 | 203.48 | C |
| ATOM | 6913 | O | ILE | B | 1921 | −25.796 | −34.099 | 72.489 | 1.00 | 203.29 | O |
| ATOM | 6914 | CB | ILE | B | 1921 | −28.364 | −33.468 | 73.388 | 1.00 | 203.52 | C |
| ATOM | 6915 | CG1 | ILE | B | 1921 | −29.763 | −33.464 | 74.006 | 1.00 | 203.54 | C |
| ATOM | 6916 | CG2 | ILE | B | 1921 | −28.391 | −34.133 | 72.008 | 1.00 | 203.42 | C |
| ATOM | 6917 | CD1 | ILE | B | 1921 | −30.812 | −32.775 | 73.167 | 1.00 | 203.54 | C |
| ATOM | 6918 | N | ASN | B | 1922 | −24.926 | −34.044 | 74.569 | 1.00 | 203.54 | N |
| ATOM | 6919 | CA | ASN | B | 1922 | −23.519 | −33.941 | 74.190 | 1.00 | 203.78 | C |
| ATOM | 6920 | C | ASN | B | 1922 | −22.735 | −33.066 | 75.145 | 1.00 | 203.83 | C |
| ATOM | 6921 | O | ASN | B | 1922 | −21.659 | −33.437 | 75.617 | 1.00 | 203.75 | O |
| ATOM | 6922 | CB | ASN | B | 1922 | −23.358 | −33.362 | 72.788 | 1.00 | 203.96 | C |
| ATOM | 6923 | CG | ASN | B | 1922 | −21.984 | −33.637 | 72.187 | 1.00 | 204.66 | C |
| ATOM | 6924 | OD1 | ASN | B | 1922 | −21.139 | −34.322 | 72.782 | 1.00 | 204.96 | O |
| ATOM | 6925 | ND2 | ASN | B | 1922 | −21.760 | −33.107 | 70.988 | 1.00 | 205.18 | N |
| ATOM | 6926 | N | GLY | B | 1923 | −23.278 | −31.890 | 75.414 | 1.00 | 203.92 | N |
| ATOM | 6927 | CA | GLY | B | 1923 | −22.540 | −30.874 | 76.126 | 1.00 | 204.40 | C |
| ATOM | 6928 | C | GLY | B | 1923 | −22.382 | −29.640 | 75.262 | 1.00 | 204.78 | C |
| ATOM | 6929 | O | GLY | B | 1923 | −21.615 | −28.731 | 75.618 | 1.00 | 205.10 | O |
| ATOM | 6930 | N | TYR | B | 1924 | −23.063 | −29.641 | 74.103 | 1.00 | 204.84 | N |
| ATOM | 6931 | CA | TYR | B | 1924 | −23.447 | −28.407 | 73.363 | 1.00 | 204.61 | C |
| ATOM | 6932 | C | TYR | B | 1924 | −24.914 | −28.123 | 73.670 | 1.00 | 204.38 | C |
| ATOM | 6933 | O | TYR | B | 1924 | −25.647 | −27.518 | 72.868 | 1.00 | 204.36 | O |
| ATOM | 6934 | CB | TYR | B | 1924 | −23.254 | −28.551 | 71.856 | 1.00 | 204.49 | C |
| ATOM | 6935 | CG | TYR | B | 1924 | −21.938 | −28.037 | 71.397 | 1.00 | 204.29 | C |
| ATOM | 6936 | CD1 | TYR | B | 1924 | −21.701 | −26.673 | 71.319 | 1.00 | 204.39 | C |
| ATOM | 6937 | CD2 | TYR | B | 1924 | −20.916 | −28.913 | 71.059 | 1.00 | 204.26 | C |
| ATOM | 6938 | CE1 | TYR | B | 1924 | −20.470 | −26.185 | 70.901 | 1.00 | 205.48 | C |
| ATOM | 6939 | CE2 | TYR | B | 1924 | −19.680 | −28.447 | 70.634 | 1.00 | 204.99 | C |
| ATOM | 6940 | CZ | TYR | B | 1924 | −19.459 | −27.079 | 70.555 | 1.00 | 205.50 | C |
| ATOM | 6941 | OH | TYR | B | 1924 | −18.230 | −26.608 | 70.130 | 1.00 | 205.54 | O |
| ATOM | 6942 | N | ILE | B | 1925 | −25.306 | −28.569 | 74.864 | 1.00 | 203.87 | N |
| ATOM | 6943 | CA | ILE | B | 1925 | −26.690 | −28.754 | 75.227 | 1.00 | 203.18 | C |
| ATOM | 6944 | C | ILE | B | 1925 | −27.120 | −27.679 | 76.181 | 1.00 | 202.81 | C |
| ATOM | 6945 | O | ILE | B | 1925 | −27.340 | −27.841 | 77.387 | 1.00 | 202.56 | O |
| ATOM | 6946 | CB | ILE | B | 1925 | −26.954 | −30.154 | 75.744 | 1.00 | 203.24 | C |
| ATOM | 6947 | CG1 | ILE | B | 1925 | −25.974 | −31.123 | 75.084 | 1.00 | 202.37 | C |
| ATOM | 6948 | CG2 | ILE | B | 1925 | −28.413 | −30.524 | 75.490 | 1.00 | 203.36 | C |
| ATOM | 6949 | CD1 | ILE | B | 1925 | −25.990 | −31.109 | 73.570 | 1.00 | 202.14 | C |
| ATOM | 6950 | N | MET | B | 1926 | −27.162 | −26.531 | 75.565 | 1.00 | 202.54 | N |
| ATOM | 6951 | CA | MET | B | 1926 | −28.034 | −25.504 | 75.956 | 1.00 | 202.48 | C |
| ATOM | 6952 | C | MET | B | 1926 | −29.389 | −26.060 | 75.538 | 1.00 | 201.81 | C |
| ATOM | 6953 | O | MET | B | 1926 | −29.814 | −27.127 | 75.982 | 1.00 | 201.49 | O |
| ATOM | 6954 | CB | MET | B | 1926 | −27.656 | −24.238 | 75.158 | 1.00 | 203.11 | C |
| ATOM | 6955 | CG | MET | B | 1926 | −26.705 | −24.458 | 73.918 | 1.00 | 203.59 | C |
| ATOM | 6956 | SD | MET | B | 1926 | −24.922 | −24.099 | 74.134 | 1.00 | 203.80 | S |
| ATOM | 6957 | CE | MET | B | 1926 | −24.301 | −24.559 | 72.516 | 1.00 | 203.77 | C |
| ATOM | 6958 | N | ASP | B | 1927 | −30.045 | −25.322 | 74.662 | 1.00 | 201.23 | N |
| ATOM | 6959 | CA | ASP | B | 1927 | −31.257 | −25.743 | 74.025 | 1.00 | 200.56 | C |
| ATOM | 6960 | C | ASP | B | 1927 | −30.935 | −25.587 | 72.538 | 1.00 | 200.16 | C |
| ATOM | 6961 | O | ASP | B | 1927 | −31.741 | −25.066 | 71.756 | 1.00 | 199.97 | O |
| ATOM | 6962 | CB | ASP | B | 1927 | −32.413 | −24.845 | 74.496 | 1.00 | 200.55 | C |
| ATOM | 6963 | CG | ASP | B | 1927 | −32.521 | −24.764 | 76.043 | 1.00 | 200.07 | C |
| ATOM | 6964 | OD1 | ASP | B | 1927 | −31.539 | −24.377 | 76.718 | 1.00 | 198.89 | O |
| ATOM | 6965 | OD2 | ASP | B | 1927 | −33.603 | −25.073 | 76.588 | 1.00 | 199.65 | O |
| ATOM | 6966 | N | THR | B | 1928 | −29.716 | −26.028 | 72.187 | 1.00 | 199.68 | N |
| ATOM | 6967 | CA | THR | B | 1928 | −29.166 | −25.979 | 70.815 | 1.00 | 199.27 | C |
| ATOM | 6968 | C | THR | B | 1928 | −27.691 | −26.462 | 70.630 | 1.00 | 198.87 | C |
| ATOM | 6969 | O | THR | B | 1928 | −26.742 | −25.793 | 71.055 | 1.00 | 198.55 | O |
| ATOM | 6970 | CB | THR | B | 1928 | −29.348 | −24.584 | 70.181 | 1.00 | 199.35 | C |
| ATOM | 6971 | OG1 | THR | B | 1928 | −28.621 | −24.518 | 68.947 | 1.00 | 199.81 | O |
| ATOM | 6972 | CG2 | THR | B | 1928 | −28.868 | −23.489 | 71.131 | 1.00 | 199.29 | C |
| ATOM | 6973 | N | LEU | B | 1929 | −27.525 | −27.615 | 69.972 | 1.00 | 198.48 | N |
| ATOM | 6974 | CA | LEU | B | 1929 | −26.215 | −28.154 | 69.569 | 1.00 | 197.96 | C |
| ATOM | 6975 | C | LEU | B | 1929 | −26.150 | −28.151 | 68.049 | 1.00 | 197.97 | C |
| ATOM | 6976 | O | LEU | B | 1929 | −27.197 | −28.205 | 67.398 | 1.00 | 197.94 | O |
| ATOM | 6977 | CB | LEU | B | 1929 | −26.006 | −29.586 | 70.084 | 1.00 | 197.71 | C |
| ATOM | 6978 | CG | LEU | B | 1929 | −26.061 | −30.777 | 69.112 | 1.00 | 196.80 | C |
| ATOM | 6979 | CD1 | LEU | B | 1929 | −25.175 | −31.921 | 69.574 | 1.00 | 195.79 | C |
| ATOM | 6980 | CD2 | LEU | B | 1929 | −27.480 | −31.252 | 68.865 | 1.00 | 196.24 | C |
| ATOM | 6981 | N | PRO | B | 1930 | −24.928 | −28.134 | 67.478 | 1.00 | 197.94 | N |
| ATOM | 6982 | CA | PRO | B | 1930 | −24.724 | −27.825 | 66.057 | 1.00 | 197.99 | C |
| ATOM | 6983 | C | PRO | B | 1930 | −25.122 | −28.877 | 65.001 | 1.00 | 198.08 | C |
| ATOM | 6984 | O | PRO | B | 1930 | −26.257 | −28.860 | 64.496 | 1.00 | 198.19 | O |
| ATOM | 6985 | CB | PRO | B | 1930 | −23.217 | −27.532 | 65.981 | 1.00 | 197.89 | C |
| ATOM | 6986 | CG | PRO | B | 1930 | −22.631 | −28.358 | 67.045 | 1.00 | 197.88 | C |
| ATOM | 6987 | CD | PRO | B | 1930 | −23.646 | −28.394 | 68.161 | 1.00 | 198.00 | C |
| ATOM | 6988 | N | GLY | B | 1931 | −24.189 | −29.778 | 64.688 | 1.00 | 198.08 | N |
| ATOM | 6989 | CA | GLY | B | 1931 | −24.164 | −30.494 | 63.410 | 1.00 | 197.97 | C |
| ATOM | 6990 | C | GLY | B | 1931 | −25.210 | −31.536 | 63.079 | 1.00 | 197.81 | C |
| ATOM | 6991 | O | GLY | B | 1931 | −24.883 | −32.703 | 62.925 | 1.00 | 197.78 | O |
| ATOM | 6992 | N | LEU | B | 1932 | −26.460 | −31.124 | 62.924 | 1.00 | 197.79 | N |
| ATOM | 6993 | CA | LEU | B | 1932 | −27.478 | −32.073 | 62.515 | 1.00 | 198.06 | C |
| ATOM | 6994 | C | LEU | B | 1932 | −27.944 | −31.833 | 61.080 | 1.00 | 198.36 | C |
| ATOM | 6995 | O | LEU | B | 1932 | −29.107 | −31.531 | 60.818 | 1.00 | 198.54 | O |
| ATOM | 6996 | CB | LEU | B | 1932 | −28.620 | −32.111 | 63.525 | 1.00 | 197.95 | C |
| ATOM | 6997 | CG | LEU | B | 1932 | −28.183 | −32.580 | 64.920 | 1.00 | 198.18 | C |
| ATOM | 6998 | CD1 | LEU | B | 1932 | −29.368 | −32.739 | 65.858 | 1.00 | 198.60 | C |
| ATOM | 6999 | CD2 | LEU | B | 1932 | −27.363 | −33.876 | 64.883 | 1.00 | 197.63 | C |
| ATOM | 7000 | N | VAL | B | 1933 | −26.999 | −31.976 | 60.157 | 1.00 | 198.72 | N |
| ATOM | 7001 | CA | VAL | B | 1933 | −27.241 | −31.774 | 58.731 | 1.00 | 199.16 | C |
| ATOM | 7002 | C | VAL | B | 1933 | −27.653 | −33.085 | 58.080 | 1.00 | 199.35 | C |
| ATOM | 7003 | O | VAL | B | 1933 | −26.921 | −34.070 | 58.197 | 1.00 | 199.56 | O |
| ATOM | 7004 | CB | VAL | B | 1933 | −25.957 | −31.238 | 57.993 | 1.00 | 199.34 | C |
| ATOM | 7005 | CG1 | VAL | B | 1933 | −25.625 | −29.800 | 58.424 | 1.00 | 199.66 | C |
| ATOM | 7006 | CG2 | VAL | B | 1933 | −24.723 | −32.187 | 58.158 | 1.00 | 199.08 | C |
| ATOM | 7007 | N | MET | B | 1934 | −28.805 | −33.118 | 57.404 | 1.00 | 199.49 | N |
| ATOM | 7008 | CA | MET | B | 1934 | −29.131 | −34.293 | 56.562 | 1.00 | 199.70 | C |
| ATOM | 7009 | C | MET | B | 1934 | −30.133 | −34.125 | 55.395 | 1.00 | 199.81 | C |
| ATOM | 7010 | O | MET | B | 1934 | −31.105 | −33.356 | 55.462 | 1.00 | 199.80 | O |
| ATOM | 7011 | CB | MET | B | 1934 | −29.448 | −35.552 | 57.402 | 1.00 | 199.69 | C |
| ATOM | 7012 | CG | MET | B | 1934 | −30.905 | −35.760 | 57.759 | 1.00 | 199.69 | C |
| ATOM | 7013 | SD | MET | B | 1934 | −31.309 | −35.147 | 59.385 | 1.00 | 199.37 | S |
| ATOM | 7014 | CE | MET | B | 1934 | −30.631 | −36.436 | 60.408 | 1.00 | 198.79 | C |
| ATOM | 7015 | N | ALA | B | 1935 | −29.858 | −34.893 | 54.339 | 1.00 | 199.87 | N |
| ATOM | 7016 | CA | ALA | B | 1935 | −30.641 | −34.924 | 53.109 | 1.00 | 199.95 | C |
| ATOM | 7017 | C | ALA | B | 1935 | −31.975 | −35.593 | 53.329 | 1.00 | 200.00 | C |
| ATOM | 7018 | O | ALA | B | 1935 | −32.021 | −36.755 | 53.729 | 1.00 | 200.12 | O |
| ATOM | 7019 | CB | ALA | B | 1935 | −29.878 | −35.672 | 52.043 | 1.00 | 200.03 | C |
| ATOM | 7020 | N | GLN | B | 1936 | −33.050 | −34.874 | 53.024 | 1.00 | 200.03 | N |
| ATOM | 7021 | CA | GLN | B | 1936 | −34.402 | −35.340 | 53.309 | 1.00 | 200.42 | C |
| ATOM | 7022 | C | GLN | B | 1936 | −34.772 | −36.716 | 52.718 | 1.00 | 200.50 | C |
| ATOM | 7023 | O | GLN | B | 1936 | −35.922 | −37.155 | 52.808 | 1.00 | 200.39 | O |
| ATOM | 7024 | CB | GLN | B | 1936 | −35.418 | −34.259 | 52.937 | 1.00 | 200.53 | C |
| ATOM | 7025 | CG | GLN | B | 1936 | −35.738 | −34.156 | 51.465 | 1.00 | 201.49 | C |
| ATOM | 7026 | CD | GLN | B | 1936 | −36.901 | −35.037 | 51.080 | 1.00 | 202.98 | C |
| ATOM | 7027 | OE1 | GLN | B | 1936 | −37.880 | −35.158 | 51.822 | 1.00 | 203.12 | O |
| ATOM | 7028 | NE2 | GLN | B | 1936 | −36.798 | −35.671 | 49.918 | 1.00 | 204.15 | N |
| ATOM | 7029 | N | ASP | B | 1937 | −33.793 | −37.396 | 52.131 | 1.00 | 200.82 | N |
| ATOM | 7030 | CA | ASP | B | 1937 | −33.957 | −38.803 | 51.777 | 1.00 | 201.09 | C |
| ATOM | 7031 | C | ASP | B | 1937 | −32.886 | −39.703 | 52.363 | 1.00 | 200.91 | C |
| ATOM | 7032 | O | ASP | B | 1937 | −33.194 | −40.436 | 53.300 | 1.00 | 200.69 | O |
| ATOM | 7033 | CB | ASP | B | 1937 | −34.083 | −38.993 | 50.274 | 1.00 | 201.47 | C |
| ATOM | 7034 | CG | ASP | B | 1937 | −35.467 | −38.669 | 49.778 | 1.00 | 202.30 | C |
| ATOM | 7035 | OD1 | ASP | B | 1937 | −35.564 | −38.128 | 48.649 | 1.00 | 203.91 | O |
| ATOM | 7036 | OD2 | ASP | B | 1937 | −36.447 | −38.940 | 50.524 | 1.00 | 202.13 | O |
| ATOM | 7037 | N | GLN | B | 1938 | −31.657 | −39.649 | 51.820 | 1.00 | 200.75 | N |
| ATOM | 7038 | CA | GLN | B | 1938 | −30.534 | −40.456 | 52.318 | 1.00 | 200.53 | C |
| ATOM | 7039 | C | GLN | B | 1938 | −30.844 | −40.778 | 53.761 | 1.00 | 200.38 | C |
| ATOM | 7040 | O | GLN | B | 1938 | −30.566 | −39.974 | 54.647 | 1.00 | 200.34 | O |
| ATOM | 7041 | CB | GLN | B | 1938 | −29.201 | −39.710 | 52.189 | 1.00 | 200.46 | C |
| ATOM | 7042 | CG | GLN | B | 1938 | −28.298 | −40.218 | 51.053 | 1.00 | 201.21 | C |
| ATOM | 7043 | CD | GLN | B | 1938 | −27.683 | −39.095 | 50.167 | 1.00 | 202.25 | C |
| ATOM | 7044 | OE1 | GLN | B | 1938 | −26.455 | −38.890 | 50.140 | 1.00 | 201.21 | O |
| ATOM | 7045 | NE2 | GLN | B | 1938 | −28.546 | −38.384 | 49.426 | 1.00 | 202.42 | N |
| ATOM | 7046 | N | ARG | B | 1939 | −31.475 | −41.942 | 53.961 | 1.00 | 200.24 | N |
| ATOM | 7047 | CA | ARG | B | 1939 | −32.117 | −42.354 | 55.228 | 1.00 | 200.07 | C |
| ATOM | 7048 | C | ARG | B | 1939 | −31.124 | −42.534 | 56.352 | 1.00 | 199.28 | C |
| ATOM | 7049 | O | ARG | B | 1939 | −30.151 | −43.281 | 56.224 | 1.00 | 199.31 | O |
| ATOM | 7050 | CB | ARG | B | 1939 | −32.959 | −43.634 | 55.043 | 1.00 | 200.01 | C |
| ATOM | 7051 | CG | ARG | B | 1939 | −32.519 | −44.440 | 53.752 | 1.00 | 201.29 | C |
| ATOM | 7052 | CD | ARG | B | 1939 | −33.158 | −45.831 | 53.736 | 1.00 | 201.40 | C |
| ATOM | 7053 | NE | ARG | B | 1939 | −32.495 | −46.755 | 54.665 | 1.00 | 202.93 | N |
| ATOM | 7054 | CZ | ARG | B | 1939 | −33.058 | −47.293 | 55.745 | 1.00 | 202.33 | C |
| ATOM | 7055 | NH1 | ARG | B | 1939 | −32.348 | −48.123 | 56.503 | 1.00 | 202.12 | N |
| ATOM | 7056 | NH2 | ARG | B | 1939 | −34.323 | −47.018 | 56.061 | 1.00 | 202.00 | N |
| ATOM | 7057 | N | ILE | B | 1940 | −31.410 | −41.859 | 57.461 | 1.00 | 198.43 | N |
| ATOM | 7058 | CA | ILE | B | 1940 | −30.418 | −41.554 | 58.477 | 1.00 | 197.45 | C |
| ATOM | 7059 | C | ILE | B | 1940 | −30.502 | −42.514 | 59.651 | 1.00 | 196.75 | C |
| ATOM | 7060 | O | ILE | B | 1940 | −31.588 | −42.795 | 60.166 | 1.00 | 196.45 | O |
| ATOM | 7061 | CB | ILE | B | 1940 | −30.584 | −40.097 | 58.965 | 1.00 | 197.57 | C |
| ATOM | 7062 | CG1 | ILE | B | 1940 | −31.099 | −40.045 | 60.397 | 1.00 | 198.18 | C |
| ATOM | 7063 | CG2 | ILE | B | 1940 | −31.524 | −39.308 | 58.052 | 1.00 | 197.33 | C |
| ATOM | 7064 | CD1 | ILE | B | 1940 | −29.998 | −40.090 | 61.422 | 1.00 | 199.67 | C |
| ATOM | 7065 | N | ARG | B | 1941 | −29.348 | −43.017 | 60.072 | 1.00 | 196.15 | N |
| ATOM | 7066 | CA | ARG | B | 1941 | −29.296 | −43.904 | 61.223 | 1.00 | 195.76 | C |
| ATOM | 7067 | C | ARG | B | 1941 | −28.843 | −43.115 | 62.436 | 1.00 | 195.63 | C |
| ATOM | 7068 | O | ARG | B | 1941 | −27.831 | −42.412 | 62.370 | 1.00 | 195.87 | O |
| ATOM | 7069 | CB | ARG | B | 1941 | −28.354 | −45.080 | 60.983 | 1.00 | 195.60 | C |
| ATOM | 7070 | CG | ARG | B | 1941 | −28.239 | −46.001 | 62.173 | 1.00 | 195.29 | C |
| ATOM | 7071 | CD | ARG | B | 1941 | −27.946 | −47.400 | 61.749 | 1.00 | 195.96 | C |
| ATOM | 7072 | NE | ARG | B | 1941 | −26.531 | −47.635 | 61.456 | 1.00 | 197.07 | N |
| ATOM | 7073 | CZ | ARG | B | 1941 | −25.985 | −47.701 | 60.236 | 1.00 | 196.99 | C |
| ATOM | 7074 | NH1 | ARG | B | 1941 | −26.714 | −47.525 | 59.134 | 1.00 | 196.33 | N |
| ATOM | 7075 | NH2 | ARG | B | 1941 | −24.685 | −47.945 | 60.122 | 1.00 | 196.49 | N |
| ATOM | 7076 | N | TRP | B | 1942 | −29.606 | −43.246 | 63.526 | 1.00 | 195.10 | N |
| ATOM | 7077 | CA | TRP | B | 1942 | −29.370 | −42.577 | 64.803 | 1.00 | 194.39 | C |
| ATOM | 7078 | C | TRP | B | 1942 | −28.778 | −43.528 | 65.824 | 1.00 | 194.23 | C |
| ATOM | 7079 | O | TRP | B | 1942 | −29.306 | −44.621 | 66.019 | 1.00 | 194.33 | O |
| ATOM | 7080 | CB | TRP | B | 1942 | −30.697 | −42.142 | 65.370 | 1.00 | 194.23 | C |
| ATOM | 7081 | CG | TRP | B | 1942 | −31.317 | −41.008 | 64.684 | 1.00 | 194.28 | C |
| ATOM | 7082 | CD1 | TRP | B | 1942 | −32.431 | −41.035 | 63.894 | 1.00 | 194.44 | C |
| ATOM | 7083 | CD2 | TRP | B | 1942 | −30.898 | −39.647 | 64.748 | 1.00 | 194.15 | C |
| ATOM | 7084 | NE1 | TRP | B | 1942 | −32.729 | −39.767 | 63.455 | 1.00 | 194.30 | N |
| ATOM | 7085 | CE2 | TRP | B | 1942 | −31.803 | −38.896 | 63.966 | 1.00 | 194.09 | C |
| ATOM | 7086 | CE3 | TRP | B | 1942 | −29.845 | −38.986 | 65.389 | 1.00 | 194.03 | C |
| ATOM | 7087 | CZ2 | TRP | B | 1942 | −31.682 | −37.522 | 63.804 | 1.00 | 194.07 | C |
| ATOM | 7088 | CZ3 | TRP | B | 1942 | −29.730 | −37.621 | 65.232 | 1.00 | 194.27 | C |
| ATOM | 7089 | CH2 | TRP | B | 1942 | −30.642 | −36.903 | 64.443 | 1.00 | 194.35 | C |
| ATOM | 7090 | N | TYR | B | 1943 | −27.720 | −43.095 | 66.508 | 1.00 | 194.00 | N |
| ATOM | 7091 | CA | TYR | B | 1943 | −27.087 | −43.900 | 67.556 | 1.00 | 193.88 | C |
| ATOM | 7092 | C | TYR | B | 1943 | −27.348 | −43.341 | 68.958 | 1.00 | 193.97 | C |
| ATOM | 7093 | O | TYR | B | 1943 | −26.537 | −42.585 | 69.484 | 1.00 | 194.11 | O |
| ATOM | 7094 | CB | TYR | B | 1943 | −25.586 | −43.987 | 67.315 | 1.00 | 193.69 | C |
| ATOM | 7095 | CG | TYR | B | 1943 | −25.203 | −44.839 | 66.144 | 1.00 | 193.51 | C |
| ATOM | 7096 | CD1 | TYR | B | 1943 | −25.393 | −44.393 | 64.843 | 1.00 | 193.63 | C |
| ATOM | 7097 | CD2 | TYR | B | 1943 | −24.632 | −46.085 | 66.331 | 1.00 | 193.59 | C |
| ATOM | 7098 | CE1 | TYR | B | 1943 | −25.034 | −45.172 | 63.752 | 1.00 | 193.73 | C |
| ATOM | 7099 | CE2 | TYR | B | 1943 | −24.269 | −46.876 | 65.254 | 1.00 | 194.09 | C |
| ATOM | 7100 | CZ | TYR | B | 1943 | −24.469 | −46.410 | 63.964 | 1.00 | 194.05 | C |
| ATOM | 7101 | OH | TYR | B | 1943 | −24.110 | −47.182 | 62.884 | 1.00 | 194.13 | O |
| ATOM | 7102 | N | LEU | B | 1944 | −28.468 | −43.723 | 69.567 | 1.00 | 193.90 | N |
| ATOM | 7103 | CA | LEU | B | 1944 | −28.842 | −43.196 | 70.873 | 1.00 | 194.01 | C |
| ATOM | 7104 | C | LEU | B | 1944 | −28.267 | −44.027 | 72.026 | 1.00 | 194.62 | C |
| ATOM | 7105 | O | LEU | B | 1944 | −28.614 | −45.212 | 72.166 | 1.00 | 194.86 | O |
| ATOM | 7106 | CB | LEU | B | 1944 | −30.357 | −43.140 | 70.983 | 1.00 | 193.67 | C |
| ATOM | 7107 | CG | LEU | B | 1944 | −31.078 | −42.477 | 69.817 | 1.00 | 193.45 | C |
| ATOM | 7108 | CD1 | LEU | B | 1944 | −32.526 | −42.886 | 69.797 | 1.00 | 193.92 | C |
| ATOM | 7109 | CD2 | LEU | B | 1944 | −30.962 | −40.977 | 69.887 | 1.00 | 193.60 | C |
| ATOM | 7110 | N | LEU | B | 1945 | −27.381 | −43.414 | 72.832 | 1.00 | 195.07 | N |
| ATOM | 7111 | CA | LEU | B | 1945 | −26.837 | −44.034 | 74.079 | 1.00 | 195.09 | C |
| ATOM | 7112 | C | LEU | B | 1945 | −26.943 | −43.076 | 75.304 | 1.00 | 195.46 | C |
| ATOM | 7113 | O | LEU | B | 1945 | −26.202 | −42.090 | 75.418 | 1.00 | 195.20 | O |
| ATOM | 7114 | CB | LEU | B | 1945 | −25.392 | −44.656 | 73.920 | 1.00 | 194.75 | C |
| ATOM | 7115 | CG | LEU | B | 1945 | −24.538 | −45.087 | 72.675 | 1.00 | 192.82 | C |
| ATOM | 7116 | CD1 | LEU | B | 1945 | −23.382 | −45.980 | 73.068 | 1.00 | 190.80 | C |
| ATOM | 7117 | CD2 | LEU | B | 1945 | −25.260 | −45.739 | 71.528 | 1.00 | 190.55 | C |
| ATOM | 7118 | N | SER | B | 1946 | −27.903 | −43.368 | 76.186 | 1.00 | 196.14 | N |
| ATOM | 7119 | CA | SER | B | 1946 | −28.053 | −42.686 | 77.481 | 1.00 | 197.01 | C |
| ATOM | 7120 | C | SER | B | 1946 | −27.061 | −43.235 | 78.504 | 1.00 | 197.77 | C |
| ATOM | 7121 | O | SER | B | 1946 | −26.735 | −44.432 | 78.493 | 1.00 | 197.95 | O |
| ATOM | 7122 | CB | SER | B | 1946 | −29.474 | −42.844 | 78.034 | 1.00 | 196.85 | C |
| ATOM | 7123 | OG | SER | B | 1946 | −29.553 | −42.492 | 79.410 | 1.00 | 196.44 | O |
| ATOM | 7124 | N | MET | B | 1947 | −26.607 | −42.354 | 79.397 | 1.00 | 198.40 | N |
| ATOM | 7125 | CA | MET | B | 1947 | −25.593 | −42.705 | 80.386 | 1.00 | 198.89 | C |
| ATOM | 7126 | C | MET | B | 1947 | −25.950 | −42.223 | 81.789 | 1.00 | 199.14 | C |
| ATOM | 7127 | O | MET | B | 1947 | −27.086 | −42.409 | 82.226 | 1.00 | 199.34 | O |
| ATOM | 7128 | CB | MET | B | 1947 | −24.217 | −42.197 | 79.940 | 1.00 | 199.01 | C |
| ATOM | 7129 | CG | MET | B | 1947 | −23.398 | −43.270 | 79.249 | 1.00 | 199.44 | C |
| ATOM | 7130 | SD | MET | B | 1947 | −23.183 | −44.701 | 80.342 | 1.00 | 200.41 | S |
| ATOM | 7131 | CE | MET | B | 1947 | −22.852 | −46.026 | 79.172 | 1.00 | 200.00 | C |
| ATOM | 7132 | N | GLY | B | 1948 | −24.967 | −41.653 | 82.494 | 1.00 | 199.34 | N |
| ATOM | 7133 | CA | GLY | B | 1948 | −25.174 | −40.960 | 83.778 | 1.00 | 199.34 | C |
| ATOM | 7134 | C | GLY | B | 1948 | −25.436 | −41.761 | 85.049 | 1.00 | 199.28 | C |
| ATOM | 7135 | O | GLY | B | 1948 | −24.673 | −42.678 | 85.396 | 1.00 | 199.23 | O |
| ATOM | 7136 | N | SER | B | 1949 | −26.521 | −41.383 | 85.738 | 1.00 | 199.14 | N |
| ATOM | 7137 | CA | SER | B | 1949 | −26.889 | −41.905 | 87.067 | 1.00 | 198.91 | C |
| ATOM | 7138 | C | SER | B | 1949 | −28.140 | −42.788 | 87.030 | 1.00 | 198.50 | C |
| ATOM | 7139 | O | SER | B | 1949 | −28.402 | −43.468 | 86.035 | 1.00 | 198.46 | O |
| ATOM | 7140 | CB | SER | B | 1949 | −27.094 | −40.751 | 88.065 | 1.00 | 199.04 | C |
| ATOM | 7141 | OG | SER | B | 1949 | −25.925 | −39.955 | 88.209 | 1.00 | 199.37 | O |
| ATOM | 7142 | N | ASN | B | 1950 | −28.911 | −42.772 | 88.115 | 1.00 | 197.95 | N |
| ATOM | 7143 | CA | ASN | B | 1950 | −30.076 | −43.634 | 88.206 | 1.00 | 197.58 | C |
| ATOM | 7144 | C | ASN | B | 1950 | −31.417 | −42.917 | 88.064 | 1.00 | 197.32 | C |
| ATOM | 7145 | O | ASN | B | 1950 | −32.483 | −43.502 | 88.284 | 1.00 | 197.28 | O |
| ATOM | 7146 | CB | ASN | B | 1950 | −30.029 | −44.471 | 89.476 | 1.00 | 197.65 | C |
| ATOM | 7147 | CG | ASN | B | 1950 | −30.552 | −45.875 | 89.258 | 1.00 | 197.88 | C |
| ATOM | 7148 | OD1 | ASN | B | 1950 | −31.152 | −46.172 | 88.221 | 1.00 | 197.92 | O |
| ATOM | 7149 | ND2 | ASN | B | 1950 | −30.321 | −46.754 | 90.231 | 1.00 | 198.04 | N |
| ATOM | 7150 | N | GLU | B | 1951 | −31.357 | −41.646 | 87.692 | 1.00 | 196.98 | N |
| ATOM | 7151 | CA | GLU | B | 1951 | −32.551 | −40.931 | 87.271 | 1.00 | 196.64 | C |
| ATOM | 7152 | C | GLU | B | 1951 | −32.424 | −40.666 | 85.779 | 1.00 | 195.91 | C |
| ATOM | 7153 | O | GLU | B | 1951 | −33.326 | −40.130 | 85.147 | 1.00 | 195.76 | O |
| ATOM | 7154 | CB | GLU | B | 1951 | −32.728 | −39.630 | 88.077 | 1.00 | 196.97 | C |
| ATOM | 7155 | CG | GLU | B | 1951 | −32.459 | −38.298 | 87.335 | 1.00 | 197.97 | C |
| ATOM | 7156 | CD | GLU | B | 1951 | −30.986 | −37.917 | 87.258 | 1.00 | 199.33 | C |
| ATOM | 7157 | OE1 | GLU | B | 1951 | −30.654 | −36.761 | 87.620 | 1.00 | 199.24 | O |
| ATOM | 7158 | OE2 | GLU | B | 1951 | −30.165 | −38.760 | 86.825 | 1.00 | 200.30 | O |
| ATOM | 7159 | N | ASN | B | 1952 | −31.292 | −41.071 | 85.223 | 1.00 | 195.22 | N |
| ATOM | 7160 | CA | ASN | B | 1952 | −30.963 | −40.763 | 83.845 | 1.00 | 194.74 | C |
| ATOM | 7161 | C | ASN | B | 1952 | −31.688 | −41.575 | 82.763 | 1.00 | 194.35 | C |
| ATOM | 7162 | O | ASN | B | 1952 | −31.112 | −41.888 | 81.707 | 1.00 | 194.42 | O |
| ATOM | 7163 | CB | ASN | B | 1952 | −29.453 | −40.804 | 83.654 | 1.00 | 194.86 | C |
| ATOM | 7164 | CG | ASN | B | 1952 | −28.856 | −39.429 | 83.511 | 1.00 | 195.06 | C |
| ATOM | 7165 | OD1 | ASN | B | 1952 | −28.769 | −38.891 | 82.405 | 1.00 | 195.51 | O |
| ATOM | 7166 | ND2 | ASN | B | 1952 | −28.430 | −38.850 | 84.626 | 1.00 | 195.10 | N |
| ATOM | 7167 | N | ILE | B | 1953 | −32.947 | −41.921 | 83.030 | 1.00 | 193.63 | N |
| ATOM | 7168 | CA | ILE | B | 1953 | −33.851 | −42.363 | 81.974 | 1.00 | 192.83 | C |
| ATOM | 7169 | C | ILE | B | 1953 | −34.114 | −41.128 | 81.146 | 1.00 | 192.50 | C |
| ATOM | 7170 | O | ILE | B | 1953 | −34.326 | −40.045 | 81.686 | 1.00 | 192.70 | O |
| ATOM | 7171 | CB | ILE | B | 1953 | −35.214 | −42.823 | 82.502 | 1.00 | 192.66 | C |
| ATOM | 7172 | CG1 | ILE | B | 1953 | −35.058 | −43.579 | 83.821 | 1.00 | 192.83 | C |
| ATOM | 7173 | CG2 | ILE | B | 1953 | −35.937 | −43.644 | 81.433 | 1.00 | 192.34 | C |
| ATOM | 7174 | CD1 | ILE | B | 1953 | −36.246 | −43.444 | 84.760 | 1.00 | 193.36 | C |
| ATOM | 7175 | N | HIS | B | 1954 | −34.078 | −41.272 | 79.834 | 1.00 | 191.90 | N |
| ATOM | 7176 | CA | HIS | B | 1954 | −34.475 | −40.178 | 78.976 | 1.00 | 191.19 | C |
| ATOM | 7177 | C | HIS | B | 1954 | −35.323 | −40.739 | 77.853 | 1.00 | 190.80 | C |
| ATOM | 7178 | O | HIS | B | 1954 | −34.971 | −41.736 | 77.220 | 1.00 | 190.63 | O |
| ATOM | 7179 | CB | HIS | B | 1954 | −33.260 | −39.409 | 78.460 | 1.00 | 191.22 | C |
| ATOM | 7180 | CG | HIS | B | 1954 | −32.474 | −38.719 | 79.536 | 1.00 | 191.02 | C |
| ATOM | 7181 | ND1 | HIS | B | 1954 | −32.634 | −37.382 | 79.834 | 1.00 | 191.01 | N |
| ATOM | 7182 | CD2 | HIS | B | 1954 | −31.518 | −39.179 | 80.376 | 1.00 | 190.80 | C |
| ATOM | 7183 | CE1 | HIS | B | 1954 | −31.810 | −37.049 | 80.810 | 1.00 | 191.01 | C |
| ATOM | 7184 | NE2 | HIS | B | 1954 | −31.126 | −38.123 | 81.162 | 1.00 | 191.06 | N |
| ATOM | 7185 | N | SER | B | 1955 | −36.470 | −40.120 | 77.641 | 1.00 | 190.37 | N |
| ATOM | 7186 | CA | SER | B | 1955 | −37.387 | −40.602 | 76.637 | 1.00 | 190.05 | C |
| ATOM | 7187 | C | SER | B | 1955 | −37.448 | −39.568 | 75.511 | 1.00 | 189.83 | C |
| ATOM | 7188 | O | SER | B | 1955 | −38.136 | −38.551 | 75.627 | 1.00 | 189.64 | O |
| ATOM | 7189 | CB | SER | B | 1955 | −38.750 | −40.913 | 77.269 | 1.00 | 190.13 | C |
| ATOM | 7190 | OG | SER | B | 1955 | −38.660 | −41.964 | 78.232 | 1.00 | 189.62 | O |
| ATOM | 7191 | N | ILE | B | 1956 | −36.683 | −39.846 | 74.445 | 1.00 | 189.67 | N |
| ATOM | 7192 | CA | ILE | B | 1956 | −36.453 | −38.933 | 73.310 | 1.00 | 189.36 | C |
| ATOM | 7193 | C | ILE | B | 1956 | −37.352 | −39.273 | 72.150 | 1.00 | 189.40 | C |
| ATOM | 7194 | O | ILE | B | 1956 | −37.651 | −40.454 | 71.906 | 1.00 | 189.09 | O |
| ATOM | 7195 | CB | ILE | B | 1956 | −35.027 | −39.061 | 72.682 | 1.00 | 189.20 | C |
| ATOM | 7196 | CG1 | ILE | B | 1956 | −34.125 | −39.991 | 73.487 | 1.00 | 189.00 | C |
| ATOM | 7197 | CG2 | ILE | B | 1956 | −34.411 | −37.687 | 72.410 | 1.00 | 188.90 | C |
| ATOM | 7198 | CD1 | ILE | B | 1956 | −33.391 | −40.966 | 72.633 | 1.00 | 187.97 | C |
| ATOM | 7199 | N | HIS | B | 1957 | −37.748 | −38.220 | 71.427 | 1.00 | 189.55 | N |
| ATOM | 7200 | CA | HIS | B | 1957 | −38.355 | −38.350 | 70.101 | 1.00 | 189.69 | C |
| ATOM | 7201 | C | HIS | B | 1957 | −38.018 | −37.182 | 69.186 | 1.00 | 189.56 | C |
| ATOM | 7202 | O | HIS | B | 1957 | −37.465 | −36.162 | 69.623 | 1.00 | 189.53 | O |
| ATOM | 7203 | CB | HIS | B | 1957 | −39.872 | −38.575 | 70.177 | 1.00 | 189.77 | C |
| ATOM | 7204 | CG | HIS | B | 1957 | −40.697 | −37.353 | 69.921 | 1.00 | 190.02 | C |
| ATOM | 7205 | ND1 | HIS | B | 1957 | −40.608 | −36.215 | 70.695 | 1.00 | 190.17 | N |
| ATOM | 7206 | CD2 | HIS | B | 1957 | −41.649 | −37.106 | 68.991 | 1.00 | 190.44 | C |
| ATOM | 7207 | CE1 | HIS | B | 1957 | −41.464 | −35.315 | 70.244 | 1.00 | 190.78 | C |
| ATOM | 7208 | NE2 | HIS | B | 1957 | −42.109 | −35.831 | 69.212 | 1.00 | 191.05 | N |
| ATOM | 7209 | N | PHE | B | 1958 | −38.370 | −37.362 | 67.916 | 1.00 | 189.32 | N |
| ATOM | 7210 | CA | PHE | B | 1958 | −38.111 | −36.399 | 66.870 | 1.00 | 189.09 | C |
| ATOM | 7211 | C | PHE | B | 1958 | −39.453 | −35.976 | 66.320 | 1.00 | 189.35 | C |
| ATOM | 7212 | O | PHE | B | 1958 | −40.154 | −36.789 | 65.697 | 1.00 | 189.42 | O |
| ATOM | 7213 | CB | PHE | B | 1958 | −37.310 | −37.079 | 65.772 | 1.00 | 188.71 | C |
| ATOM | 7214 | CG | PHE | B | 1958 | −36.236 | −37.979 | 66.283 | 1.00 | 187.50 | C |
| ATOM | 7215 | CD1 | PHE | B | 1958 | −34.919 | −37.547 | 66.329 | 1.00 | 186.57 | C |
| ATOM | 7216 | CD2 | PHE | B | 1958 | −36.537 | −39.257 | 66.713 | 1.00 | 186.18 | C |
| ATOM | 7217 | CE1 | PHE | B | 1958 | −33.916 | −38.370 | 66.800 | 1.00 | 186.19 | C |
| ATOM | 7218 | CE2 | PHE | B | 1958 | −35.539 | −40.085 | 67.187 | 1.00 | 186.37 | C |
| ATOM | 7219 | CZ | PHE | B | 1958 | −34.224 | −39.641 | 67.234 | 1.00 | 186.59 | C |
| ATOM | 7220 | N | SER | B | 1959 | −39.820 | −34.717 | 66.553 | 1.00 | 189.43 | N |
| ATOM | 7221 | CA | SER | B | 1959 | −41.155 | −34.261 | 66.192 | 1.00 | 189.81 | C |
| ATOM | 7222 | C | SER | B | 1959 | −41.455 | −34.569 | 64.736 | 1.00 | 190.39 | C |
| ATOM | 7223 | O | SER | B | 1959 | −40.563 | −34.502 | 63.886 | 1.00 | 190.46 | O |
| ATOM | 7224 | CB | SER | B | 1959 | −41.322 | −32.773 | 66.461 | 1.00 | 189.57 | C |
| ATOM | 7225 | OG | SER | B | 1959 | −42.502 | −32.267 | 65.862 | 1.00 | 189.24 | O |
| ATOM | 7226 | N | GLY | B | 1960 | −42.705 | −34.940 | 64.464 | 1.00 | 191.04 | N |
| ATOM | 7227 | CA | GLY | B | 1960 | −43.187 | −35.139 | 63.092 | 1.00 | 191.81 | C |
| ATOM | 7228 | C | GLY | B | 1960 | −42.317 | −36.046 | 62.242 | 1.00 | 192.18 | C |
| ATOM | 7229 | O | GLY | B | 1960 | −42.146 | −35.829 | 61.026 | 1.00 | 192.54 | O |
| ATOM | 7230 | N | HIS | B | 1961 | −41.762 | −37.053 | 62.906 | 1.00 | 192.14 | N |
| ATOM | 7231 | CA | HIS | B | 1961 | −40.918 | −38.042 | 62.287 | 1.00 | 192.23 | C |
| ATOM | 7232 | C | HIS | B | 1961 | −41.197 | −39.330 | 63.033 | 1.00 | 192.31 | C |
| ATOM | 7233 | O | HIS | B | 1961 | −41.661 | −39.290 | 64.178 | 1.00 | 192.54 | O |
| ATOM | 7234 | CB | HIS | B | 1961 | −39.441 | −37.659 | 62.439 | 1.00 | 192.20 | C |
| ATOM | 7235 | CG | HIS | B | 1961 | −38.918 | −36.759 | 61.357 | 1.00 | 192.23 | C |
| ATOM | 7236 | ND1 | HIS | B | 1961 | −37.944 | −35.810 | 61.586 | 1.00 | 191.37 | N |
| ATOM | 7237 | CD2 | HIS | B | 1961 | −39.222 | −36.672 | 60.037 | 1.00 | 192.66 | C |
| ATOM | 7238 | CE1 | HIS | B | 1961 | −37.672 | −35.179 | 60.458 | 1.00 | 191.73 | C |
| ATOM | 7239 | NE2 | HIS | B | 1961 | −38.436 | −35.679 | 59.503 | 1.00 | 192.36 | N |
| ATOM | 7240 | N | VAL | B | 1962 | −40.925 | −40.461 | 62.383 | 1.00 | 192.23 | N |
| ATOM | 7241 | CA | VAL | B | 1962 | −41.059 | −41.785 | 62.992 | 1.00 | 191.90 | C |
| ATOM | 7242 | C | VAL | B | 1962 | −39.924 | −42.651 | 62.524 | 1.00 | 191.92 | C |
| ATOM | 7243 | O | VAL | B | 1962 | −39.487 | −42.550 | 61.369 | 1.00 | 192.03 | O |
| ATOM | 7244 | CB | VAL | B | 1962 | −42.379 | −42.461 | 62.589 | 1.00 | 191.88 | C |
| ATOM | 7245 | CG1 | VAL | B | 1962 | −42.286 | −43.980 | 62.668 | 1.00 | 191.30 | C |
| ATOM | 7246 | CG2 | VAL | B | 1962 | −43.503 | −41.956 | 63.452 | 1.00 | 192.29 | C |
| ATOM | 7247 | N | PHE | B | 1963 | −39.471 | −43.523 | 63.413 | 1.00 | 191.99 | N |
| ATOM | 7248 | CA | PHE | B | 1963 | −38.339 | −44.397 | 63.124 | 1.00 | 192.40 | C |
| ATOM | 7249 | C | PHE | B | 1963 | −38.613 | −45.869 | 63.402 | 1.00 | 192.50 | C |
| ATOM | 7250 | O | PHE | B | 1963 | −39.550 | −46.224 | 64.116 | 1.00 | 192.65 | O |
| ATOM | 7251 | CB | PHE | B | 1963 | −37.133 | −43.946 | 63.927 | 1.00 | 192.50 | C |
| ATOM | 7252 | CG | PHE | B | 1963 | −37.410 | −43.796 | 65.382 | 1.00 | 192.65 | C |
| ATOM | 7253 | CD1 | PHE | B | 1963 | −38.355 | −42.885 | 65.829 | 1.00 | 193.06 | C |
| ATOM | 7254 | CD2 | PHE | B | 1963 | −36.720 | −44.564 | 66.311 | 1.00 | 192.60 | C |
| ATOM | 7255 | CE1 | PHE | B | 1963 | −38.607 | −42.753 | 67.179 | 1.00 | 193.83 | C |
| ATOM | 7256 | CE2 | PHE | B | 1963 | −36.956 | −44.442 | 67.662 | 1.00 | 192.48 | C |
| ATOM | 7257 | CZ | PHE | B | 1963 | −37.903 | −43.537 | 68.103 | 1.00 | 193.26 | C |
| ATOM | 7258 | N | THR | B | 1964 | −37.781 | −46.727 | 62.834 | 1.00 | 192.54 | N |
| ATOM | 7259 | CA | THR | B | 1964 | −37.996 | −48.149 | 62.950 | 1.00 | 192.82 | C |
| ATOM | 7260 | C | THR | B | 1964 | −36.864 | −48.725 | 63.797 | 1.00 | 192.95 | C |
| ATOM | 7261 | O | THR | B | 1964 | −35.727 | −48.277 | 63.667 | 1.00 | 192.80 | O |
| ATOM | 7262 | CB | THR | B | 1964 | −38.150 | −48.804 | 61.529 | 1.00 | 192.96 | C |
| ATOM | 7263 | OG1 | THR | B | 1964 | −38.267 | −50.223 | 61.645 | 1.00 | 193.41 | O |
| ATOM | 7264 | CG2 | THR | B | 1964 | −36.991 | −48.456 | 60.566 | 1.00 | 193.14 | C |
| ATOM | 7265 | N | VAL | B | 1965 | −37.177 | −49.653 | 64.710 | 1.00 | 193.35 | N |
| ATOM | 7266 | CA | VAL | B | 1965 | −36.129 | −50.373 | 65.461 | 1.00 | 193.70 | C |
| ATOM | 7267 | C | VAL | B | 1965 | −36.193 | −51.860 | 65.221 | 1.00 | 194.34 | C |
| ATOM | 7268 | O | VAL | B | 1965 | −37.274 | −52.439 | 65.121 | 1.00 | 194.10 | O |
| ATOM | 7269 | CB | VAL | B | 1965 | −36.165 | −50.207 | 66.996 | 1.00 | 193.44 | C |
| ATOM | 7270 | CG1 | VAL | B | 1965 | −34.748 | −50.070 | 67.514 | 1.00 | 193.05 | C |
| ATOM | 7271 | CG2 | VAL | B | 1965 | −37.005 | −49.037 | 67.433 | 1.00 | 193.70 | C |
| ATOM | 7272 | N | ARG | B | 1966 | −35.007 | −52.457 | 65.159 | 1.00 | 195.42 | N |
| ATOM | 7273 | CA | ARG | B | 1966 | −34.815 | −53.886 | 64.978 | 1.00 | 196.73 | C |
| ATOM | 7274 | C | ARG | B | 1966 | −34.511 | −54.545 | 66.324 | 1.00 | 196.84 | C |
| ATOM | 7275 | O | ARG | B | 1966 | −34.124 | −53.879 | 67.283 | 1.00 | 197.08 | O |
| ATOM | 7276 | CB | ARG | B | 1966 | −33.657 | −54.133 | 64.008 | 1.00 | 197.19 | C |
| ATOM | 7277 | CG | ARG | B | 1966 | −32.268 | −54.246 | 64.681 | 1.00 | 200.59 | C |
| ATOM | 7278 | CD | ARG | B | 1966 | −31.659 | −52.904 | 65.186 | 1.00 | 205.80 | C |
| ATOM | 7279 | NE | ARG | B | 1966 | −30.854 | −53.067 | 66.410 | 1.00 | 209.16 | N |
| ATOM | 7280 | CZ | ARG | B | 1966 | −29.539 | −53.314 | 66.463 | 1.00 | 210.70 | C |
| ATOM | 7281 | NH1 | ARG | B | 1966 | −28.801 | −53.433 | 65.354 | 1.00 | 210.70 | N |
| ATOM | 7282 | NH2 | ARG | B | 1966 | −28.955 | −53.439 | 67.653 | 1.00 | 211.85 | N |
| ATOM | 7283 | N | LYS | B | 1967 | −34.662 | −55.857 | 66.384 | 1.00 | 197.11 | N |
| ATOM | 7284 | CA | LYS | B | 1967 | −34.533 | −56.582 | 67.625 | 1.00 | 197.67 | C |
| ATOM | 7285 | C | LYS | B | 1967 | −34.696 | −58.012 | 67.154 | 1.00 | 197.79 | C |
| ATOM | 7286 | O | LYS | B | 1967 | −33.866 | −58.530 | 66.413 | 1.00 | 197.67 | O |
| ATOM | 7287 | CB | LYS | B | 1967 | −35.676 | −56.173 | 68.589 | 1.00 | 197.98 | C |
| ATOM | 7288 | CG | LYS | B | 1967 | −35.413 | −56.245 | 70.133 | 1.00 | 198.93 | C |
| ATOM | 7289 | CD | LYS | B | 1967 | −35.754 | −57.640 | 70.787 | 1.00 | 199.97 | C |
| ATOM | 7290 | CE | LYS | B | 1967 | −37.268 | −57.909 | 71.038 | 1.00 | 199.70 | C |
| ATOM | 7291 | NZ | LYS | B | 1967 | −37.709 | −57.825 | 72.471 | 1.00 | 199.05 | N |
| ATOM | 7292 | N | LYS | B | 1968 | −35.786 | −58.634 | 67.577 | 1.00 | 198.27 | N |
| ATOM | 7293 | CA | LYS | B | 1968 | −36.267 | −59.855 | 66.976 | 1.00 | 198.72 | C |
| ATOM | 7294 | C | LYS | B | 1968 | −36.498 | −59.522 | 65.499 | 1.00 | 198.69 | C |
| ATOM | 7295 | O | LYS | B | 1968 | −35.787 | −60.037 | 64.630 | 1.00 | 198.66 | O |
| ATOM | 7296 | CB | LYS | B | 1968 | −37.567 | −60.307 | 67.679 | 1.00 | 199.07 | C |
| ATOM | 7297 | CG | LYS | B | 1968 | −37.416 | −61.380 | 68.787 | 1.00 | 199.31 | C |
| ATOM | 7298 | CD | LYS | B | 1968 | −37.569 | −62.786 | 68.182 | 1.00 | 200.00 | C |
| ATOM | 7299 | CE | LYS | B | 1968 | −37.202 | −63.893 | 69.150 | 1.00 | 199.89 | C |
| ATOM | 7300 | NZ | LYS | B | 1968 | −36.981 | −65.193 | 68.452 | 1.00 | 199.76 | N |
| ATOM | 7301 | N | GLU | B | 1969 | −37.467 | −58.634 | 65.244 | 1.00 | 198.66 | N |
| ATOM | 7302 | CA | GLU | B | 1969 | −37.752 | −58.095 | 63.911 | 1.00 | 198.67 | C |
| ATOM | 7303 | C | GLU | B | 1969 | −37.485 | −56.610 | 63.874 | 1.00 | 198.61 | C |
| ATOM | 7304 | O | GLU | B | 1969 | −36.592 | −56.108 | 64.551 | 1.00 | 198.56 | O |
| ATOM | 7305 | CB | GLU | B | 1969 | −39.211 | −58.323 | 63.519 | 1.00 | 198.57 | C |
| ATOM | 7306 | CG | GLU | B | 1969 | −39.455 | −59.626 | 62.813 | 1.00 | 199.12 | C |
| ATOM | 7307 | CD | GLU | B | 1969 | −39.382 | −60.803 | 63.758 | 1.00 | 199.81 | C |
| ATOM | 7308 | OE1 | GLU | B | 1969 | −38.513 | −61.678 | 63.545 | 1.00 | 200.56 | O |
| ATOM | 7309 | OE2 | GLU | B | 1969 | −40.180 | −60.843 | 64.725 | 1.00 | 199.88 | O |
| ATOM | 7310 | N | GLU | B | 1970 | −38.269 | −55.918 | 63.058 | 1.00 | 198.61 | N |
| ATOM | 7311 | CA | GLU | B | 1970 | −38.268 | −54.475 | 63.024 | 1.00 | 198.85 | C |
| ATOM | 7312 | C | GLU | B | 1970 | −39.693 | −54.046 | 63.295 | 1.00 | 198.63 | C |
| ATOM | 7313 | O | GLU | B | 1970 | −40.613 | −54.439 | 62.574 | 1.00 | 198.34 | O |
| ATOM | 7314 | CB | GLU | B | 1970 | −37.756 | −53.943 | 61.670 | 1.00 | 199.05 | C |
| ATOM | 7315 | CG | GLU | B | 1970 | −36.201 | −54.013 | 61.499 | 1.00 | 199.82 | C |
| ATOM | 7316 | CD | GLU | B | 1970 | −35.666 | −53.624 | 60.097 | 1.00 | 199.43 | C |
| ATOM | 7317 | OE1 | GLU | B | 1970 | −34.502 | −54.019 | 59.799 | 1.00 | 198.77 | O |
| ATOM | 7318 | OE2 | GLU | B | 1970 | −36.392 | −52.937 | 59.318 | 1.00 | 199.55 | O |
| ATOM | 7319 | N | TYR | B | 1971 | −39.882 | −53.291 | 64.374 | 1.00 | 198.73 | N |
| ATOM | 7320 | CA | TYR | B | 1971 | −41.182 | −52.681 | 64.628 | 1.00 | 198.88 | C |
| ATOM | 7321 | C | TYR | B | 1971 | −41.134 | −51.182 | 64.899 | 1.00 | 198.54 | C |
| ATOM | 7322 | O | TYR | B | 1971 | −40.158 | −50.675 | 65.451 | 1.00 | 198.31 | O |
| ATOM | 7323 | CB | TYR | B | 1971 | −42.112 | −53.517 | 65.556 | 1.00 | 199.49 | C |
| ATOM | 7324 | CG | TYR | B | 1971 | −41.797 | −53.798 | 67.045 | 1.00 | 200.26 | C |
| ATOM | 7325 | CD1 | TYR | B | 1971 | −40.542 | −54.248 | 67.491 | 1.00 | 200.41 | C |
| ATOM | 7326 | CD2 | TYR | B | 1971 | −42.826 | −53.707 | 68.001 | 1.00 | 201.48 | C |
| ATOM | 7327 | CE1 | TYR | B | 1971 | −40.315 | −54.542 | 68.875 | 1.00 | 200.19 | C |
| ATOM | 7328 | CE2 | TYR | B | 1971 | −42.611 | −53.993 | 69.364 | 1.00 | 201.28 | C |
| ATOM | 7329 | CZ | TYR | B | 1971 | −41.363 | −54.406 | 69.798 | 1.00 | 200.65 | C |
| ATOM | 7330 | OH | TYR | B | 1971 | −41.197 | −54.666 | 71.147 | 1.00 | 200.14 | O |
| ATOM | 7331 | N | LYS | B | 1972 | −42.186 | −50.495 | 64.449 | 1.00 | 198.30 | N |
| ATOM | 7332 | CA | LYS | B | 1972 | −42.182 | −49.043 | 64.253 | 1.00 | 198.08 | C |
| ATOM | 7333 | C | LYS | B | 1972 | −42.552 | −48.298 | 65.511 | 1.00 | 197.79 | C |
| ATOM | 7334 | O | LYS | B | 1972 | −43.523 | −48.648 | 66.173 | 1.00 | 197.73 | O |
| ATOM | 7335 | CB | LYS | B | 1972 | −43.172 | −48.661 | 63.156 | 1.00 | 198.20 | C |
| ATOM | 7336 | CG | LYS | B | 1972 | −42.724 | −47.509 | 62.266 | 1.00 | 198.56 | C |
| ATOM | 7337 | CD | LYS | B | 1972 | −41.786 | −48.008 | 61.165 | 1.00 | 199.57 | C |
| ATOM | 7338 | CE | LYS | B | 1972 | −41.951 | −47.242 | 59.859 | 1.00 | 200.42 | C |
| ATOM | 7339 | NZ | LYS | B | 1972 | −43.382 | −47.152 | 59.421 | 1.00 | 200.85 | N |
| ATOM | 7340 | N | MET | B | 1973 | −41.787 | −47.251 | 65.813 | 1.00 | 197.48 | N |
| ATOM | 7341 | CA | MET | B | 1973 | −41.893 | −46.537 | 67.088 | 1.00 | 197.37 | C |
| ATOM | 7342 | C | MET | B | 1973 | −41.704 | −45.013 | 66.963 | 1.00 | 197.00 | C |
| ATOM | 7343 | O | MET | B | 1973 | −41.020 | −44.538 | 66.053 | 1.00 | 197.04 | O |
| ATOM | 7344 | CB | MET | B | 1973 | −40.911 | −47.145 | 68.103 | 1.00 | 197.39 | C |
| ATOM | 7345 | CG | MET | B | 1973 | −41.393 | −48.483 | 68.703 | 1.00 | 197.80 | C |
| ATOM | 7346 | SD | MET | B | 1973 | −40.183 | −49.394 | 69.696 | 1.00 | 197.97 | S |
| ATOM | 7347 | CE | MET | B | 1973 | −41.273 | −50.450 | 70.681 | 1.00 | 197.62 | C |
| ATOM | 7348 | N | ALA | B | 1974 | −42.316 | −44.257 | 67.876 | 1.00 | 196.61 | N |
| ATOM | 7349 | CA | ALA | B | 1974 | −42.319 | −42.792 | 67.805 | 1.00 | 196.30 | C |
| ATOM | 7350 | C | ALA | B | 1974 | −41.372 | −42.106 | 68.787 | 1.00 | 196.30 | C |
| ATOM | 7351 | O | ALA | B | 1974 | −40.950 | −40.973 | 68.535 | 1.00 | 196.20 | O |
| ATOM | 7352 | CB | ALA | B | 1974 | −43.708 | −42.270 | 67.987 | 1.00 | 196.19 | C |
| ATOM | 7353 | N | LEU | B | 1975 | −41.064 | −42.780 | 69.901 | 1.00 | 196.35 | N |
| ATOM | 7354 | CA | LEU | B | 1975 | −40.070 | −42.309 | 70.883 | 1.00 | 196.49 | C |
| ATOM | 7355 | C | LEU | B | 1975 | −39.532 | −43.430 | 71.764 | 1.00 | 196.68 | C |
| ATOM | 7356 | O | LEU | B | 1975 | −40.232 | −44.413 | 72.023 | 1.00 | 196.53 | O |
| ATOM | 7357 | CB | LEU | B | 1975 | −40.635 | −41.184 | 71.749 | 1.00 | 196.53 | C |
| ATOM | 7358 | CG | LEU | B | 1975 | −41.508 | −41.368 | 72.997 | 1.00 | 196.84 | C |
| ATOM | 7359 | CD1 | LEU | B | 1975 | −42.626 | −42.391 | 72.795 | 1.00 | 197.10 | C |
| ATOM | 7360 | CD2 | LEU | B | 1975 | −40.647 | −41.699 | 74.212 | 1.00 | 197.20 | C |
| ATOM | 7361 | N | TYR | B | 1976 | −38.306 | −43.262 | 72.255 | 1.00 | 197.13 | N |
| ATOM | 7362 | CA | TYR | B | 1976 | −37.588 | −44.373 | 72.892 | 1.00 | 197.76 | C |
| ATOM | 7363 | C | TYR | B | 1976 | −36.975 | −44.048 | 74.256 | 1.00 | 198.08 | C |
| ATOM | 7364 | O | TYR | B | 1976 | −35.987 | −43.308 | 74.356 | 1.00 | 198.26 | O |
| ATOM | 7365 | CB | TYR | B | 1976 | −36.491 | −44.889 | 71.954 | 1.00 | 197.90 | C |
| ATOM | 7366 | CG | TYR | B | 1976 | −36.157 | −46.367 | 72.073 | 1.00 | 197.92 | C |
| ATOM | 7367 | CD1 | TYR | B | 1976 | −35.281 | −46.833 | 73.058 | 1.00 | 197.73 | C |
| ATOM | 7368 | CD2 | TYR | B | 1976 | −36.691 | −47.292 | 71.169 | 1.00 | 197.76 | C |
| ATOM | 7369 | CE1 | TYR | B | 1976 | −34.971 | −48.180 | 73.154 | 1.00 | 197.87 | C |
| ATOM | 7370 | CE2 | TYR | B | 1976 | −36.381 | −48.639 | 71.252 | 1.00 | 197.74 | C |
| ATOM | 7371 | CZ | TYR | B | 1976 | −35.521 | −49.077 | 72.245 | 1.00 | 197.97 | C |
| ATOM | 7372 | OH | TYR | B | 1976 | −35.222 | −50.417 | 72.328 | 1.00 | 198.49 | O |
| ATOM | 7373 | N | ASN | B | 1977 | −37.560 | −44.632 | 75.299 | 1.00 | 198.29 | N |
| ATOM | 7374 | CA | ASN | B | 1977 | −37.029 | −44.546 | 76.645 | 1.00 | 198.41 | C |
| ATOM | 7375 | C | ASN | B | 1977 | −35.638 | −45.116 | 76.634 | 1.00 | 198.45 | C |
| ATOM | 7376 | O | ASN | B | 1977 | −35.436 | −46.316 | 76.827 | 1.00 | 198.51 | O |
| ATOM | 7377 | CB | ASN | B | 1977 | −37.892 | −45.345 | 77.609 | 1.00 | 198.52 | C |
| ATOM | 7378 | CG | ASN | B | 1977 | −39.336 | −45.392 | 77.179 | 1.00 | 199.02 | C |
| ATOM | 7379 | OD1 | ASN | B | 1977 | −39.717 | −46.169 | 76.273 | 1.00 | 199.10 | O |
| ATOM | 7380 | ND2 | ASN | B | 1977 | −40.157 | −44.552 | 77.824 | 1.00 | 199.44 | N |
| ATOM | 7381 | N | LEU | B | 1978 | −34.680 | −44.252 | 76.343 | 1.00 | 198.54 | N |
| ATOM | 7382 | CA | LEU | B | 1978 | −33.296 | −44.594 | 76.515 | 1.00 | 198.80 | C |
| ATOM | 7383 | C | LEU | B | 1978 | −33.083 | −44.960 | 77.971 | 1.00 | 198.83 | C |
| ATOM | 7384 | O | LEU | B | 1978 | −33.609 | −44.299 | 78.872 | 1.00 | 198.99 | O |
| ATOM | 7385 | CB | LEU | B | 1978 | −32.420 | −43.401 | 76.154 | 1.00 | 198.90 | C |
| ATOM | 7386 | CG | LEU | B | 1978 | −31.551 | −43.508 | 74.909 | 1.00 | 199.46 | C |
| ATOM | 7387 | CD1 | LEU | B | 1978 | −30.650 | −42.277 | 74.752 | 1.00 | 199.49 | C |
| ATOM | 7388 | CD2 | LEU | B | 1978 | −30.718 | −44.784 | 74.984 | 1.00 | 200.59 | C |
| ATOM | 7389 | N | TYR | B | 1979 | −32.337 | −46.025 | 78.211 | 1.00 | 198.71 | N |
| ATOM | 7390 | CA | TYR | B | 1979 | −31.968 | −46.339 | 79.571 | 1.00 | 198.77 | C |
| ATOM | 7391 | C | TYR | B | 1979 | −30.484 | −46.118 | 79.758 | 1.00 | 199.09 | C |
| ATOM | 7392 | O | TYR | B | 1979 | −29.760 | −46.024 | 78.774 | 1.00 | 199.21 | O |
| ATOM | 7393 | CB | TYR | B | 1979 | −32.421 | −47.749 | 79.933 | 1.00 | 198.49 | C |
| ATOM | 7394 | CG | TYR | B | 1979 | −33.821 | −47.723 | 80.493 | 1.00 | 198.51 | C |
| ATOM | 7395 | CD1 | TYR | B | 1979 | −34.928 | −47.564 | 79.656 | 1.00 | 198.79 | C |
| ATOM | 7396 | CD2 | TYR | B | 1979 | −34.043 | −47.802 | 81.867 | 1.00 | 198.49 | C |
| ATOM | 7397 | CE1 | TYR | B | 1979 | −36.221 | −47.509 | 80.174 | 1.00 | 198.26 | C |
| ATOM | 7398 | CE2 | TYR | B | 1979 | −35.332 | −47.746 | 82.392 | 1.00 | 197.96 | C |
| ATOM | 7399 | CZ | TYR | B | 1979 | −36.408 | −47.601 | 81.538 | 1.00 | 197.85 | C |
| ATOM | 7400 | OH | TYR | B | 1979 | −37.668 | −47.549 | 82.055 | 1.00 | 197.40 | O |
| ATOM | 7401 | N | PRO | B | 1980 | −30.028 | −45.960 | 81.012 | 1.00 | 199.42 | N |
| ATOM | 7402 | CA | PRO | B | 1980 | −28.587 | −45.939 | 81.188 | 1.00 | 199.76 | C |
| ATOM | 7403 | C | PRO | B | 1980 | −28.013 | −47.338 | 80.935 | 1.00 | 200.19 | C |
| ATOM | 7404 | O | PRO | B | 1980 | −28.409 | −48.311 | 81.605 | 1.00 | 199.99 | O |
| ATOM | 7405 | CB | PRO | B | 1980 | −28.414 | −45.508 | 82.646 | 1.00 | 199.71 | C |
| ATOM | 7406 | CG | PRO | B | 1980 | −29.658 | −45.924 | 83.310 | 1.00 | 199.56 | C |
| ATOM | 7407 | CD | PRO | B | 1980 | −30.740 | −45.773 | 82.286 | 1.00 | 199.44 | C |
| ATOM | 7408 | N | GLY | B | 1981 | −27.126 | −47.417 | 79.935 | 1.00 | 200.69 | N |
| ATOM | 7409 | CA | GLY | B | 1981 | −26.434 | −48.653 | 79.540 | 1.00 | 201.18 | C |
| ATOM | 7410 | C | GLY | B | 1981 | −26.999 | −49.377 | 78.326 | 1.00 | 201.56 | C |
| ATOM | 7411 | O | GLY | B | 1981 | −26.499 | −50.433 | 77.946 | 1.00 | 201.38 | O |
| ATOM | 7412 | N | VAL | B | 1982 | −28.047 | −48.813 | 77.728 | 1.00 | 202.13 | N |
| ATOM | 7413 | CA | VAL | B | 1982 | −28.732 | −49.419 | 76.585 | 1.00 | 202.71 | C |
| ATOM | 7414 | C | VAL | B | 1982 | −28.459 | −48.584 | 75.344 | 1.00 | 203.05 | C |
| ATOM | 7415 | O | VAL | B | 1982 | −28.927 | −47.455 | 75.195 | 1.00 | 202.83 | O |
| ATOM | 7416 | CB | VAL | B | 1982 | −30.255 | −49.619 | 76.847 | 1.00 | 202.81 | C |
| ATOM | 7417 | CG1 | VAL | B | 1982 | −31.049 | −49.630 | 75.549 | 1.00 | 203.18 | C |
| ATOM | 7418 | CG2 | VAL | B | 1982 | −30.502 | −50.910 | 77.642 | 1.00 | 202.93 | C |
| ATOM | 7419 | N | PHE | B | 1983 | −27.679 | −49.175 | 74.455 | 1.00 | 203.86 | N |
| ATOM | 7420 | CA | PHE | B | 1983 | −27.047 | −48.449 | 73.366 | 1.00 | 204.55 | C |
| ATOM | 7421 | C | PHE | B | 1983 | −27.833 | −48.701 | 72.094 | 1.00 | 203.91 | C |
| ATOM | 7422 | O | PHE | B | 1983 | −27.634 | −49.701 | 71.382 | 1.00 | 203.90 | O |
| ATOM | 7423 | CB | PHE | B | 1983 | −25.568 | −48.860 | 73.239 | 1.00 | 205.59 | C |
| ATOM | 7424 | CG | PHE | B | 1983 | −24.908 | −49.219 | 74.574 | 1.00 | 208.09 | C |
| ATOM | 7425 | CD1 | PHE | B | 1983 | −24.682 | −48.232 | 75.567 | 1.00 | 210.07 | C |
| ATOM | 7426 | CD2 | PHE | B | 1983 | −24.525 | −50.545 | 74.849 | 1.00 | 209.26 | C |
| ATOM | 7427 | CE1 | PHE | B | 1983 | −24.076 | −48.564 | 76.815 | 1.00 | 209.96 | C |
| ATOM | 7428 | CE2 | PHE | B | 1983 | −23.920 | −50.883 | 76.084 | 1.00 | 209.42 | C |
| ATOM | 7429 | CZ | PHE | B | 1983 | −23.694 | −49.888 | 77.069 | 1.00 | 208.81 | C |
| ATOM | 7430 | N | GLU | B | 1984 | −28.752 | −47.780 | 71.839 | 1.00 | 203.02 | N |
| ATOM | 7431 | CA | GLU | B | 1984 | −29.709 | −47.935 | 70.768 | 1.00 | 202.23 | C |
| ATOM | 7432 | C | GLU | B | 1984 | −29.190 | −47.595 | 69.383 | 1.00 | 201.71 | C |
| ATOM | 7433 | O | GLU | B | 1984 | −28.488 | −46.600 | 69.202 | 1.00 | 201.78 | O |
| ATOM | 7434 | CB | GLU | B | 1984 | −30.975 | −47.139 | 71.075 | 1.00 | 202.13 | C |
| ATOM | 7435 | CG | GLU | B | 1984 | −31.917 | −47.891 | 71.963 | 1.00 | 202.01 | C |
| ATOM | 7436 | CD | GLU | B | 1984 | −32.030 | −49.363 | 71.581 | 1.00 | 202.79 | C |
| ATOM | 7437 | OE1 | GLU | B | 1984 | −31.529 | −49.784 | 70.509 | 1.00 | 203.00 | O |
| ATOM | 7438 | OE2 | GLU | B | 1984 | −32.618 | −50.120 | 72.369 | 1.00 | 203.78 | O |
| ATOM | 7439 | N | THR | B | 1985 | −29.529 | −48.437 | 68.411 | 1.00 | 200.88 | N |
| ATOM | 7440 | CA | THR | B | 1985 | −29.483 | −48.023 | 67.013 | 1.00 | 200.00 | C |
| ATOM | 7441 | C | THR | B | 1985 | −30.891 | −48.056 | 66.426 | 1.00 | 199.40 | C |
| ATOM | 7442 | O | THR | B | 1985 | −31.630 | −49.044 | 66.572 | 1.00 | 199.21 | O |
| ATOM | 7443 | CB | THR | B | 1985 | −28.535 | −48.865 | 66.151 | 1.00 | 199.96 | C |
| ATOM | 7444 | OG1 | THR | B | 1985 | −28.857 | −50.246 | 66.313 | 1.00 | 200.30 | O |
| ATOM | 7445 | CG2 | THR | B | 1985 | −27.101 | −48.633 | 66.552 | 1.00 | 199.50 | C |
| ATOM | 7446 | N | VAL | B | 1986 | −31.251 | −46.951 | 65.780 | 1.00 | 198.60 | N |
| ATOM | 7447 | CA | VAL | B | 1986 | −32.539 | −46.807 | 65.132 | 1.00 | 197.84 | C |
| ATOM | 7448 | C | VAL | B | 1986 | −32.423 | −45.943 | 63.864 | 1.00 | 197.76 | C |
| ATOM | 7449 | O | VAL | B | 1986 | −31.665 | −44.972 | 63.847 | 1.00 | 197.90 | O |
| ATOM | 7450 | CB | VAL | B | 1986 | −33.574 | −46.246 | 66.109 | 1.00 | 197.60 | C |
| ATOM | 7451 | CG1 | VAL | B | 1986 | −33.186 | −44.856 | 66.587 | 1.00 | 196.93 | C |
| ATOM | 7452 | CG2 | VAL | B | 1986 | −34.909 | −46.239 | 65.463 | 1.00 | 197.39 | C |
| ATOM | 7453 | N | GLU | B | 1987 | −33.165 | −46.314 | 62.814 | 1.00 | 197.37 | N |
| ATOM | 7454 | CA | GLU | B | 1987 | −33.122 | −45.647 | 61.499 | 1.00 | 196.84 | C |
| ATOM | 7455 | C | GLU | B | 1987 | −34.479 | −45.039 | 61.092 | 1.00 | 196.42 | C |
| ATOM | 7456 | O | GLU | B | 1987 | −35.545 | −45.552 | 61.463 | 1.00 | 196.26 | O |
| ATOM | 7457 | CB | GLU | B | 1987 | −32.691 | −46.638 | 60.411 | 1.00 | 196.74 | C |
| ATOM | 7458 | CG | GLU | B | 1987 | −31.255 | −47.166 | 60.466 | 1.00 | 196.78 | C |
| ATOM | 7459 | CD | GLU | B | 1987 | −31.040 | −48.371 | 59.526 | 1.00 | 197.29 | C |
| ATOM | 7460 | OE1 | GLU | B | 1987 | −29.963 | −48.509 | 58.893 | 1.00 | 197.46 | O |
| ATOM | 7461 | OE2 | GLU | B | 1987 | −31.971 | −49.194 | 59.410 | 1.00 | 198.58 | O |
| ATOM | 7462 | N | MET | B | 1988 | −34.424 | −43.958 | 60.310 | 1.00 | 196.03 | N |
| ATOM | 7463 | CA | MET | B | 1988 | −35.627 | −43.284 | 59.795 | 1.00 | 195.63 | C |
| ATOM | 7464 | C | MET | B | 1988 | −35.423 | −42.574 | 58.455 | 1.00 | 195.73 | C |
| ATOM | 7465 | O | MET | B | 1988 | −34.298 | −42.231 | 58.080 | 1.00 | 195.78 | O |
| ATOM | 7466 | CB | MET | B | 1988 | −36.133 | −42.267 | 60.798 | 1.00 | 195.67 | C |
| ATOM | 7467 | CG | MET | B | 1988 | −35.060 | −41.346 | 61.308 | 1.00 | 195.03 | C |
| ATOM | 7468 | SD | MET | B | 1988 | −35.794 | −39.824 | 61.876 | 1.00 | 195.04 | S |
| ATOM | 7469 | CE | MET | B | 1988 | −36.762 | −40.367 | 63.282 | 1.00 | 195.19 | C |
| ATOM | 7470 | N | LEU | B | 1989 | −36.535 | −42.330 | 57.765 | 1.00 | 195.62 | N |
| ATOM | 7471 | CA | LEU | B | 1989 | −36.534 | −41.776 | 56.415 | 1.00 | 195.44 | C |
| ATOM | 7472 | C | LEU | B | 1989 | −37.271 | −40.433 | 56.442 | 1.00 | 195.60 | C |
| ATOM | 7473 | O | LEU | B | 1989 | −38.464 | −40.382 | 56.144 | 1.00 | 195.81 | O |
| ATOM | 7474 | CB | LEU | B | 1989 | −37.227 | −42.767 | 55.480 | 1.00 | 195.11 | C |
| ATOM | 7475 | CG | LEU | B | 1989 | −36.914 | −42.789 | 53.995 | 1.00 | 194.80 | C |
| ATOM | 7476 | CD1 | LEU | B | 1989 | −37.007 | −44.219 | 53.483 | 1.00 | 194.71 | C |
| ATOM | 7477 | CD2 | LEU | B | 1989 | −37.860 | −41.884 | 53.242 | 1.00 | 194.81 | C |
| ATOM | 7478 | N | PRO | B | 1990 | −36.557 | −39.338 | 56.785 | 1.00 | 195.60 | N |
| ATOM | 7479 | CA | PRO | B | 1990 | −37.148 | −38.055 | 57.191 | 1.00 | 195.69 | C |
| ATOM | 7480 | C | PRO | B | 1990 | −37.870 | −37.295 | 56.089 | 1.00 | 196.01 | C |
| ATOM | 7481 | O | PRO | B | 1990 | −37.654 | −36.094 | 55.943 | 1.00 | 195.91 | O |
| ATOM | 7482 | CB | PRO | B | 1990 | −35.932 | −37.250 | 57.648 | 1.00 | 195.59 | C |
| ATOM | 7483 | CG | PRO | B | 1990 | −34.808 | −37.811 | 56.883 | 1.00 | 195.37 | C |
| ATOM | 7484 | CD | PRO | B | 1990 | −35.085 | −39.271 | 56.774 | 1.00 | 195.56 | C |
| ATOM | 7485 | N | SER | B | 1991 | −38.754 | −37.982 | 55.365 | 1.00 | 196.66 | N |
| ATOM | 7486 | CA | SER | B | 1991 | −39.416 | −37.450 | 54.158 | 1.00 | 197.36 | C |
| ATOM | 7487 | C | SER | B | 1991 | −40.298 | −36.182 | 54.337 | 1.00 | 197.79 | C |
| ATOM | 7488 | O | SER | B | 1991 | −41.139 | −35.868 | 53.478 | 1.00 | 198.13 | O |
| ATOM | 7489 | CB | SER | B | 1991 | −40.189 | −38.568 | 53.418 | 1.00 | 197.37 | C |
| ATOM | 7490 | OG | SER | B | 1991 | −39.502 | −39.004 | 52.246 | 1.00 | 197.23 | O |
| ATOM | 7491 | N | LYS | B | 1992 | −40.102 | −35.456 | 55.438 | 1.00 | 197.97 | N |
| ATOM | 7492 | CA | LYS | B | 1992 | −40.722 | −34.141 | 55.606 | 1.00 | 197.87 | C |
| ATOM | 7493 | C | LYS | B | 1992 | −39.614 | −33.090 | 55.654 | 1.00 | 197.53 | C |
| ATOM | 7494 | O | LYS | B | 1992 | −38.778 | −33.079 | 56.559 | 1.00 | 197.28 | O |
| ATOM | 7495 | CB | LYS | B | 1992 | −41.652 | −34.103 | 56.842 | 1.00 | 198.17 | C |
| ATOM | 7496 | CG | LYS | B | 1992 | −43.064 | −34.727 | 56.620 | 1.00 | 198.50 | C |
| ATOM | 7497 | CD | LYS | B | 1992 | −44.029 | −33.762 | 55.876 | 1.00 | 198.58 | C |
| ATOM | 7498 | CE | LYS | B | 1992 | −44.871 | −34.458 | 54.784 | 1.00 | 197.95 | C |
| ATOM | 7499 | NZ | LYS | B | 1992 | −45.946 | −35.367 | 55.279 | 1.00 | 196.75 | N |
| ATOM | 7500 | N | ALA | B | 1993 | −39.595 | −32.238 | 54.638 | 1.00 | 197.33 | N |
| ATOM | 7501 | CA | ALA | B | 1993 | −38.548 | −31.236 | 54.493 | 1.00 | 197.21 | C |
| ATOM | 7502 | C | ALA | B | 1993 | −38.786 | −30.065 | 55.430 | 1.00 | 197.09 | C |
| ATOM | 7503 | O | ALA | B | 1993 | −39.894 | −29.880 | 55.936 | 1.00 | 197.03 | O |
| ATOM | 7504 | CB | ALA | B | 1993 | −38.474 | −30.761 | 53.054 | 1.00 | 197.26 | C |
| ATOM | 7505 | N | GLY | B | 1994 | −37.743 | −29.273 | 55.649 | 1.00 | 196.95 | N |
| ATOM | 7506 | CA | GLY | B | 1994 | −37.812 | −28.170 | 56.597 | 1.00 | 196.99 | C |
| ATOM | 7507 | C | GLY | B | 1994 | −36.920 | −28.410 | 57.804 | 1.00 | 196.94 | C |
| ATOM | 7508 | O | GLY | B | 1994 | −35.980 | −29.203 | 57.738 | 1.00 | 197.07 | O |
| ATOM | 7509 | N | ILE | B | 1995 | −37.237 | −27.743 | 58.915 | 1.00 | 196.71 | N |
| ATOM | 7510 | CA | ILE | B | 1995 | −36.331 | −27.615 | 60.068 | 1.00 | 196.25 | C |
| ATOM | 7511 | C | ILE | B | 1995 | −36.978 | −28.078 | 61.375 | 1.00 | 196.01 | C |
| ATOM | 7512 | O | ILE | B | 1995 | −37.948 | −27.473 | 61.832 | 1.00 | 196.16 | O |
| ATOM | 7513 | CB | ILE | B | 1995 | −35.793 | −26.133 | 60.181 | 1.00 | 196.26 | C |
| ATOM | 7514 | CG1 | ILE | B | 1995 | −35.248 | −25.817 | 61.573 | 1.00 | 196.06 | C |
| ATOM | 7515 | CG2 | ILE | B | 1995 | −36.847 | −25.107 | 59.756 | 1.00 | 196.06 | C |
| ATOM | 7516 | CD1 | ILE | B | 1995 | −33.806 | −26.221 | 61.745 | 1.00 | 196.58 | C |
| ATOM | 7517 | N | TRP | B | 1996 | −36.447 | −29.139 | 61.980 | 1.00 | 195.56 | N |
| ATOM | 7518 | CA | TRP | B | 1996 | −37.052 | −29.676 | 63.208 | 1.00 | 195.36 | C |
| ATOM | 7519 | C | TRP | B | 1996 | −36.137 | −29.690 | 64.468 | 1.00 | 195.40 | C |
| ATOM | 7520 | O | TRP | B | 1996 | −35.241 | −28.848 | 64.596 | 1.00 | 195.39 | O |
| ATOM | 7521 | CB | TRP | B | 1996 | −37.610 | −31.076 | 62.966 | 1.00 | 195.23 | C |
| ATOM | 7522 | CG | TRP | B | 1996 | −38.487 | −31.296 | 61.770 | 1.00 | 194.83 | C |
| ATOM | 7523 | CD1 | TRP | B | 1996 | −38.171 | −32.018 | 60.673 | 1.00 | 194.96 | C |
| ATOM | 7524 | CD2 | TRP | B | 1996 | −39.842 | −30.866 | 61.587 | 1.00 | 194.73 | C |
| ATOM | 7525 | NE1 | TRP | B | 1996 | −39.223 | −32.053 | 59.802 | 1.00 | 195.04 | N |
| ATOM | 7526 | CE2 | TRP | B | 1996 | −40.266 | −31.352 | 60.338 | 1.00 | 194.58 | C |
| ATOM | 7527 | CE3 | TRP | B | 1996 | −40.730 | −30.101 | 62.347 | 1.00 | 195.50 | C |
| ATOM | 7528 | CZ2 | TRP | B | 1996 | −41.536 | −31.102 | 59.825 | 1.00 | 194.81 | C |
| ATOM | 7529 | CZ3 | TRP | B | 1996 | −41.997 | −29.848 | 61.833 | 1.00 | 195.37 | C |
| ATOM | 7530 | CH2 | TRP | B | 1996 | −42.384 | −30.349 | 60.583 | 1.00 | 195.06 | C |
| ATOM | 7531 | N | ARG | B | 1997 | −36.391 | −30.638 | 65.389 | 1.00 | 195.36 | N |
| ATOM | 7532 | CA | ARG | B | 1997 | −35.632 | −30.812 | 66.667 | 1.00 | 195.26 | C |
| ATOM | 7533 | C | ARG | B | 1997 | −35.823 | −32.170 | 67.419 | 1.00 | 194.92 | C |
| ATOM | 7534 | O | ARG | B | 1997 | −36.804 | −32.902 | 67.195 | 1.00 | 194.53 | O |
| ATOM | 7535 | CB | ARG | B | 1997 | −35.835 | −29.577 | 67.592 | 1.00 | 195.48 | C |
| ATOM | 7536 | CG | ARG | B | 1997 | −36.253 | −29.797 | 69.080 | 1.00 | 195.88 | C |
| ATOM | 7537 | CD | ARG | B | 1997 | −37.772 | −29.909 | 69.281 | 1.00 | 196.37 | C |
| ATOM | 7538 | NE | ARG | B | 1997 | −38.528 | −29.539 | 68.080 | 1.00 | 197.67 | N |
| ATOM | 7539 | CZ | ARG | B | 1997 | −38.986 | −30.397 | 67.161 | 1.00 | 198.09 | C |
| ATOM | 7540 | NH1 | ARG | B | 1997 | −39.646 | −29.935 | 66.104 | 1.00 | 197.80 | N |
| ATOM | 7541 | NH2 | ARG | B | 1997 | −38.788 | −31.712 | 67.280 | 1.00 | 197.80 | N |
| ATOM | 7542 | N | VAL | B | 1998 | −34.849 | −32.501 | 68.276 | 1.00 | 194.67 | N |
| ATOM | 7543 | CA | VAL | B | 1998 | −34.936 | −33.624 | 69.224 | 1.00 | 194.54 | C |
| ATOM | 7544 | C | VAL | B | 1998 | −35.127 | −33.086 | 70.640 | 1.00 | 194.64 | C |
| ATOM | 7545 | O | VAL | B | 1998 | −34.502 | −32.082 | 71.007 | 1.00 | 194.70 | O |
| ATOM | 7546 | CB | VAL | B | 1998 | −33.669 | −34.513 | 69.229 | 1.00 | 194.45 | C |
| ATOM | 7547 | CG1 | VAL | B | 1998 | −33.746 | −35.537 | 68.154 | 1.00 | 194.20 | C |
| ATOM | 7548 | CG2 | VAL | B | 1998 | −32.392 | −33.677 | 69.095 | 1.00 | 194.38 | C |
| ATOM | 7549 | N | GLU | B | 1999 | −35.980 | −33.760 | 71.425 | 1.00 | 194.47 | N |
| ATOM | 7550 | CA | GLU | B | 1999 | −36.300 | −33.355 | 72.806 | 1.00 | 194.07 | C |
| ATOM | 7551 | C | GLU | B | 1999 | −36.596 | −34.559 | 73.660 | 1.00 | 193.55 | C |
| ATOM | 7552 | O | GLU | B | 1999 | −37.360 | −35.433 | 73.242 | 1.00 | 193.42 | O |
| ATOM | 7553 | CB | GLU | B | 1999 | −37.500 | −32.387 | 72.851 | 1.00 | 194.13 | C |
| ATOM | 7554 | CG | GLU | B | 1999 | −38.884 | −32.934 | 72.396 | 1.00 | 194.39 | C |
| ATOM | 7555 | CD | GLU | B | 1999 | −39.915 | −31.818 | 72.059 | 1.00 | 194.68 | C |
| ATOM | 7556 | OE1 | GLU | B | 1999 | −41.132 | −32.120 | 71.969 | 1.00 | 195.02 | O |
| ATOM | 7557 | OE2 | GLU | B | 1999 | −39.516 | −30.639 | 71.871 | 1.00 | 195.53 | O |
| ATOM | 7558 | N | CYS | B | 2000 | −35.985 | −34.609 | 74.844 | 1.00 | 193.07 | N |
| ATOM | 7559 | CA | CYS | B | 2000 | −36.352 | −35.622 | 75.843 | 1.00 | 192.81 | C |
| ATOM | 7560 | C | CYS | B | 2000 | −37.702 | −35.248 | 76.410 | 1.00 | 192.39 | C |
| ATOM | 7561 | O | CYS | B | 2000 | −37.815 | −34.295 | 77.180 | 1.00 | 192.56 | O |
| ATOM | 7562 | CB | CYS | B | 2000 | −35.340 | −35.720 | 76.986 | 1.00 | 192.96 | C |
| ATOM | 7563 | SG | CYS | B | 2000 | −35.952 | −36.698 | 78.399 | 1.00 | 192.72 | S |
| ATOM | 7564 | N | LEU | B | 2001 | −38.723 | −36.009 | 76.045 | 1.00 | 191.67 | N |
| ATOM | 7565 | CA | LEU | B | 2001 | −40.078 | −35.574 | 76.281 | 1.00 | 190.99 | C |
| ATOM | 7566 | C | LEU | B | 2001 | −40.403 | −35.437 | 77.764 | 1.00 | 191.18 | C |
| ATOM | 7567 | O | LEU | B | 2001 | −41.470 | −34.946 | 78.117 | 1.00 | 191.46 | O |
| ATOM | 7568 | CB | LEU | B | 2001 | −41.066 | −36.496 | 75.569 | 1.00 | 190.59 | C |
| ATOM | 7569 | CG | LEU | B | 2001 | −42.224 | −35.777 | 74.877 | 1.00 | 189.27 | C |
| ATOM | 7570 | CD1 | LEU | B | 2001 | −41.714 | −34.619 | 74.064 | 1.00 | 188.75 | C |
| ATOM | 7571 | CD2 | LEU | B | 2001 | −42.995 | −36.723 | 73.992 | 1.00 | 187.88 | C |
| ATOM | 7572 | N | ILE | B | 2002 | −39.470 | −35.820 | 78.631 | 1.00 | 191.18 | N |
| ATOM | 7573 | CA | ILE | B | 2002 | −39.735 | −35.843 | 80.075 | 1.00 | 191.25 | C |
| ATOM | 7574 | C | ILE | B | 2002 | −39.860 | −34.457 | 80.710 | 1.00 | 190.99 | C |
| ATOM | 7575 | O | ILE | B | 2002 | −38.880 | −33.879 | 81.182 | 1.00 | 190.64 | O |
| ATOM | 7576 | CB | ILE | B | 2002 | −38.709 | −36.718 | 80.824 | 1.00 | 191.47 | C |
| ATOM | 7577 | CG1 | ILE | B | 2002 | −38.511 | −38.049 | 80.080 | 1.00 | 192.02 | C |
| ATOM | 7578 | CG2 | ILE | B | 2002 | −39.171 | −36.965 | 82.263 | 1.00 | 191.70 | C |
| ATOM | 7579 | CD1 | ILE | B | 2002 | −37.325 | −38.870 | 80.560 | 1.00 | 192.77 | C |
| ATOM | 7580 | N | GLY | B | 2003 | −41.092 | −33.952 | 80.717 | 1.00 | 191.02 | N |
| ATOM | 7581 | CA | GLY | B | 2003 | −41.428 | −32.610 | 81.191 | 1.00 | 191.41 | C |
| ATOM | 7582 | C | GLY | B | 2003 | −40.304 | −31.731 | 81.710 | 1.00 | 191.76 | C |
| ATOM | 7583 | O | GLY | B | 2003 | −39.918 | −30.754 | 81.065 | 1.00 | 191.73 | O |
| ATOM | 7584 | N | GLU | B | 2004 | −39.785 | −32.087 | 82.882 | 1.00 | 192.17 | N |
| ATOM | 7585 | CA | GLU | B | 2004 | −38.827 | −31.254 | 83.611 | 1.00 | 192.75 | C |
| ATOM | 7586 | C | GLU | B | 2004 | −37.453 | −31.255 | 82.958 | 1.00 | 192.60 | C |
| ATOM | 7587 | O | GLU | B | 2004 | −36.657 | −30.344 | 83.186 | 1.00 | 192.71 | O |
| ATOM | 7588 | CB | GLU | B | 2004 | −38.733 | −31.684 | 85.089 | 1.00 | 192.81 | C |
| ATOM | 7589 | CG | GLU | B | 2004 | −39.880 | −31.168 | 86.004 | 1.00 | 193.46 | C |
| ATOM | 7590 | CD | GLU | B | 2004 | −40.078 | −31.977 | 87.315 | 1.00 | 193.66 | C |
| ATOM | 7591 | OE1 | GLU | B | 2004 | −39.416 | −33.026 | 87.517 | 1.00 | 194.88 | O |
| ATOM | 7592 | OE2 | GLU | B | 2004 | −40.916 | −31.559 | 88.153 | 1.00 | 194.79 | O |
| ATOM | 7593 | N | HIS | B | 2005 | −37.184 | −32.273 | 82.146 | 1.00 | 192.55 | N |
| ATOM | 7594 | CA | HIS | B | 2005 | −35.937 | −32.335 | 81.389 | 1.00 | 192.65 | C |
| ATOM | 7595 | C | HIS | B | 2005 | −35.936 | −31.378 | 80.200 | 1.00 | 192.23 | C |
| ATOM | 7596 | O | HIS | B | 2005 | −34.953 | −30.660 | 79.978 | 1.00 | 192.12 | O |
| ATOM | 7597 | CB | HIS | B | 2005 | −35.635 | −33.767 | 80.935 | 1.00 | 193.01 | C |
| ATOM | 7598 | CG | HIS | B | 2005 | −35.221 | −34.681 | 82.049 | 1.00 | 194.26 | C |
| ATOM | 7599 | ND1 | HIS | B | 2005 | −35.261 | −36.056 | 81.939 | 1.00 | 195.10 | N |
| ATOM | 7600 | CD2 | HIS | B | 2005 | −34.766 | −34.418 | 83.300 | 1.00 | 195.13 | C |
| ATOM | 7601 | CE1 | HIS | B | 2005 | −34.843 | −36.599 | 83.069 | 1.00 | 195.40 | C |
| ATOM | 7602 | NE2 | HIS | B | 2005 | −34.537 | −35.627 | 83.912 | 1.00 | 195.58 | N |
| ATOM | 7603 | N | LEU | B | 2006 | −37.037 | −31.369 | 79.447 | 1.00 | 191.78 | N |
| ATOM | 7604 | CA | LEU | B | 2006 | −37.199 | −30.454 | 78.322 | 1.00 | 191.37 | C |
| ATOM | 7605 | C | LEU | B | 2006 | −37.024 | −29.020 | 78.788 | 1.00 | 191.39 | C |
| ATOM | 7606 | O | LEU | B | 2006 | −36.340 | −28.238 | 78.128 | 1.00 | 191.82 | O |
| ATOM | 7607 | CB | LEU | B | 2006 | −38.566 | −30.622 | 77.640 | 1.00 | 191.14 | C |
| ATOM | 7608 | CG | LEU | B | 2006 | −38.801 | −30.044 | 76.232 | 1.00 | 190.60 | C |
| ATOM | 7609 | CD1 | LEU | B | 2006 | −39.494 | −28.682 | 76.214 | 1.00 | 190.49 | C |
| ATOM | 7610 | CD2 | LEU | B | 2006 | −37.505 | −29.999 | 75.449 | 1.00 | 190.39 | C |
| ATOM | 7611 | N | HIS | B | 2007 | −37.612 | −28.682 | 79.934 | 1.00 | 191.01 | N |
| ATOM | 7612 | CA | HIS | B | 2007 | −37.627 | −27.296 | 80.390 | 1.00 | 190.64 | C |
| ATOM | 7613 | C | HIS | B | 2007 | −36.347 | −26.770 | 81.018 | 1.00 | 190.34 | C |
| ATOM | 7614 | O | HIS | B | 2007 | −36.329 | −25.680 | 81.591 | 1.00 | 190.51 | O |
| ATOM | 7615 | CB | HIS | B | 2007 | −38.846 | −27.024 | 81.254 | 1.00 | 190.74 | C |
| ATOM | 7616 | CG | HIS | B | 2007 | −40.014 | −26.544 | 80.461 | 1.00 | 191.17 | C |
| ATOM | 7617 | ND1 | HIS | B | 2007 | −40.564 | −27.283 | 79.435 | 1.00 | 191.24 | N |
| ATOM | 7618 | CD2 | HIS | B | 2007 | −40.710 | −25.384 | 80.509 | 1.00 | 191.52 | C |
| ATOM | 7619 | CE1 | HIS | B | 2007 | −41.562 | −26.606 | 78.896 | 1.00 | 191.32 | C |
| ATOM | 7620 | NE2 | HIS | B | 2007 | −41.672 | −25.452 | 79.531 | 1.00 | 191.69 | N |
| ATOM | 7621 | N | ALA | B | 2008 | −35.282 | −27.555 | 80.895 | 1.00 | 189.94 | N |
| ATOM | 7622 | CA | ALA | B | 2008 | −33.932 | −27.100 | 81.184 | 1.00 | 189.53 | C |
| ATOM | 7623 | C | ALA | B | 2008 | −32.969 | −27.837 | 80.277 | 1.00 | 189.19 | C |
| ATOM | 7624 | O | ALA | B | 2008 | −32.054 | −28.497 | 80.747 | 1.00 | 189.10 | O |
| ATOM | 7625 | CB | ALA | B | 2008 | −33.566 | −27.306 | 82.663 | 1.00 | 189.68 | C |
| ATOM | 7626 | N | GLY | B | 2009 | −33.212 | −27.746 | 78.974 | 1.00 | 188.90 | N |
| ATOM | 7627 | CA | GLY | B | 2009 | −32.215 | −28.108 | 77.973 | 1.00 | 188.66 | C |
| ATOM | 7628 | C | GLY | B | 2009 | −32.296 | −29.458 | 77.288 | 1.00 | 188.50 | C |
| ATOM | 7629 | O | GLY | B | 2009 | −31.499 | −30.350 | 77.578 | 1.00 | 188.50 | O |
| ATOM | 7630 | N | MET | B | 2010 | −33.255 | −29.606 | 76.376 | 1.00 | 188.34 | N |
| ATOM | 7631 | CA | MET | B | 2010 | −33.228 | −30.681 | 75.364 | 1.00 | 188.20 | C |
| ATOM | 7632 | C | MET | B | 2010 | −33.695 | −30.201 | 73.961 | 1.00 | 188.40 | C |
| ATOM | 7633 | O | MET | B | 2010 | −33.353 | −30.842 | 72.964 | 1.00 | 188.37 | O |
| ATOM | 7634 | CB | MET | B | 2010 | −33.950 | −31.978 | 75.827 | 1.00 | 187.53 | C |
| ATOM | 7635 | CG | MET | B | 2010 | −33.022 | −33.211 | 76.115 | 1.00 | 186.58 | C |
| ATOM | 7636 | SD | MET | B | 2010 | −32.344 | −33.431 | 77.800 | 1.00 | 183.97 | S |
| ATOM | 7637 | CE | MET | B | 2010 | −31.537 | −35.022 | 77.741 | 1.00 | 184.77 | C |
| ATOM | 7638 | N | SER | B | 2011 | −34.426 | −29.069 | 73.892 | 1.00 | 188.67 | N |
| ATOM | 7639 | CA | SER | B | 2011 | −35.036 | −28.539 | 72.624 | 1.00 | 188.56 | C |
| ATOM | 7640 | C | SER | B | 2011 | −33.975 | −28.119 | 71.591 | 1.00 | 188.74 | C |
| ATOM | 7641 | O | SER | B | 2011 | −33.803 | −26.922 | 71.278 | 1.00 | 188.65 | O |
| ATOM | 7642 | CB | SER | B | 2011 | −36.079 | −27.420 | 72.887 | 1.00 | 188.48 | C |
| ATOM | 7643 | OG | SER | B | 2011 | −36.963 | −27.257 | 71.783 | 1.00 | 187.50 | O |
| ATOM | 7644 | N | THR | B | 2012 | −33.312 | −29.147 | 71.047 | 1.00 | 188.84 | N |
| ATOM | 7645 | CA | THR | B | 2012 | −32.049 | −29.034 | 70.305 | 1.00 | 188.84 | C |
| ATOM | 7646 | C | THR | B | 2012 | −32.126 | −29.412 | 68.803 | 1.00 | 188.73 | C |
| ATOM | 7647 | O | THR | B | 2012 | −31.989 | −30.584 | 68.425 | 1.00 | 188.47 | O |
| ATOM | 7648 | CB | THR | B | 2012 | −30.953 | −29.849 | 71.019 | 1.00 | 188.86 | C |
| ATOM | 7649 | OG1 | THR | B | 2012 | −30.779 | −29.347 | 72.359 | 1.00 | 188.37 | O |
| ATOM | 7650 | CG2 | THR | B | 2012 | −29.651 | −29.774 | 70.249 | 1.00 | 188.86 | C |
| ATOM | 7651 | N | LEU | B | 2013 | −32.298 | −28.377 | 67.978 | 1.00 | 188.55 | N |
| ATOM | 7652 | CA | LEU | B | 2013 | −32.644 | −28.467 | 66.555 | 1.00 | 188.43 | C |
| ATOM | 7653 | C | LEU | B | 2013 | −31.863 | −29.451 | 65.670 | 1.00 | 187.91 | C |
| ATOM | 7654 | O | LEU | B | 2013 | −30.674 | −29.731 | 65.906 | 1.00 | 187.77 | O |
| ATOM | 7655 | CB | LEU | B | 2013 | −32.567 | −27.070 | 65.926 | 1.00 | 188.87 | C |
| ATOM | 7656 | CG | LEU | B | 2013 | −31.203 | −26.378 | 65.618 | 1.00 | 190.98 | C |
| ATOM | 7657 | CD1 | LEU | B | 2013 | −30.225 | −26.403 | 66.826 | 1.00 | 192.54 | C |
| ATOM | 7658 | CD2 | LEU | B | 2013 | −30.468 | −26.818 | 64.275 | 1.00 | 191.76 | C |
| ATOM | 7659 | N | PHE | B | 2014 | −32.573 | −29.946 | 64.646 | 1.00 | 187.27 | N |
| ATOM | 7660 | CA | PHE | B | 2014 | −31.999 | −30.612 | 63.458 | 1.00 | 186.43 | C |
| ATOM | 7661 | C | PHE | B | 2014 | −32.740 | −30.215 | 62.167 | 1.00 | 186.54 | C |
| ATOM | 7662 | O | PHE | B | 2014 | −33.964 | −30.005 | 62.160 | 1.00 | 186.44 | O |
| ATOM | 7663 | CB | PHE | B | 2014 | −31.908 | −32.140 | 63.614 | 1.00 | 185.59 | C |
| ATOM | 7664 | CG | PHE | B | 2014 | −33.169 | −32.882 | 63.284 | 1.00 | 184.05 | C |
| ATOM | 7665 | CD1 | PHE | B | 2014 | −33.166 | −33.869 | 62.321 | 1.00 | 183.50 | C |
| ATOM | 7666 | CD2 | PHE | B | 2014 | −34.347 | −32.626 | 63.953 | 1.00 | 182.79 | C |
| ATOM | 7667 | CE1 | PHE | B | 2014 | −34.329 | −34.583 | 62.020 | 1.00 | 183.10 | C |
| ATOM | 7668 | CE2 | PHE | B | 2014 | −35.499 | −33.330 | 63.658 | 1.00 | 182.70 | C |
| ATOM | 7669 | CZ | PHE | B | 2014 | −35.490 | −34.308 | 62.690 | 1.00 | 183.06 | C |
| ATOM | 7670 | N | LEU | B | 2015 | −31.974 | −30.127 | 61.083 | 1.00 | 186.52 | N |
| ATOM | 7671 | CA | LEU | B | 2015 | −32.453 | −29.602 | 59.818 | 1.00 | 186.32 | C |
| ATOM | 7672 | C | LEU | B | 2015 | −32.442 | −30.643 | 58.692 | 1.00 | 186.56 | C |
| ATOM | 7673 | O | LEU | B | 2015 | −31.464 | −31.381 | 58.529 | 1.00 | 186.51 | O |
| ATOM | 7674 | CB | LEU | B | 2015 | −31.593 | −28.409 | 59.438 | 1.00 | 185.95 | C |
| ATOM | 7675 | CG | LEU | B | 2015 | −31.298 | −28.294 | 57.953 | 1.00 | 185.57 | C |
| ATOM | 7676 | CD1 | LEU | B | 2015 | −32.322 | −27.430 | 57.215 | 1.00 | 184.72 | C |
| ATOM | 7677 | CD2 | LEU | B | 2015 | −29.891 | −27.788 | 57.776 | 1.00 | 185.83 | C |
| ATOM | 7678 | N | VAL | B | 2016 | −33.541 | −30.691 | 57.931 | 1.00 | 186.95 | N |
| ATOM | 7679 | CA | VAL | B | 2016 | −33.609 | −31.455 | 56.677 | 1.00 | 187.23 | C |
| ATOM | 7680 | C | VAL | B | 2016 | −33.889 | −30.562 | 55.466 | 1.00 | 187.81 | C |
| ATOM | 7681 | O | VAL | B | 2016 | −34.866 | −29.801 | 55.421 | 1.00 | 187.58 | O |
| ATOM | 7682 | CB | VAL | B | 2016 | −34.597 | −32.655 | 56.715 | 1.00 | 187.07 | C |
| ATOM | 7683 | CG1 | VAL | B | 2016 | −34.037 | −33.769 | 57.558 | 1.00 | 186.55 | C |
| ATOM | 7684 | CG2 | VAL | B | 2016 | −35.985 | −32.241 | 57.202 | 1.00 | 186.87 | C |
| ATOM | 7685 | N | TYR | B | 2017 | −32.987 | −30.663 | 54.496 | 1.00 | 188.66 | N |
| ATOM | 7686 | CA | TYR | B | 2017 | −33.074 | −29.938 | 53.237 | 1.00 | 189.61 | C |
| ATOM | 7687 | C | TYR | B | 2017 | −33.476 | −30.914 | 52.158 | 1.00 | 190.51 | C |
| ATOM | 7688 | O | TYR | B | 2017 | −33.288 | −32.127 | 52.319 | 1.00 | 190.67 | O |
| ATOM | 7689 | CB | TYR | B | 2017 | −31.720 | −29.307 | 52.863 | 1.00 | 189.27 | C |
| ATOM | 7690 | CG | TYR | B | 2017 | −30.532 | −30.269 | 52.751 | 1.00 | 188.93 | C |
| ATOM | 7691 | CD1 | TYR | B | 2017 | −29.991 | −30.615 | 51.508 | 1.00 | 188.90 | C |
| ATOM | 7692 | CD2 | TYR | B | 2017 | −29.932 | −30.811 | 53.893 | 1.00 | 188.50 | C |
| ATOM | 7693 | CE1 | TYR | B | 2017 | −28.886 | −31.494 | 51.412 | 1.00 | 188.73 | C |
| ATOM | 7694 | CE2 | TYR | B | 2017 | −28.836 | −31.679 | 53.808 | 1.00 | 188.17 | C |
| ATOM | 7695 | CZ | TYR | B | 2017 | −28.319 | −32.019 | 52.574 | 1.00 | 188.54 | C |
| ATOM | 7696 | OH | TYR | B | 2017 | −27.238 | −32.876 | 52.521 | 1.00 | 188.38 | O |
| ATOM | 7697 | N | SER | B | 2018 | −34.053 | −30.384 | 51.080 | 1.00 | 191.53 | N |
| ATOM | 7698 | CA | SER | B | 2018 | −34.149 | −31.119 | 49.825 | 1.00 | 192.51 | C |
| ATOM | 7699 | C | SER | B | 2018 | −32.878 | −30.767 | 49.085 | 1.00 | 193.26 | C |
| ATOM | 7700 | O | SER | B | 2018 | −32.293 | −29.711 | 49.328 | 1.00 | 193.45 | O |
| ATOM | 7701 | CB | SER | B | 2018 | −35.377 | −30.700 | 49.012 | 1.00 | 192.42 | C |
| ATOM | 7702 | OG | SER | B | 2018 | −36.522 | −31.453 | 49.381 | 1.00 | 192.42 | O |
| ATOM | 7703 | N | ASN | B | 2019 | −32.418 | −31.668 | 48.230 | 1.00 | 194.14 | N |
| ATOM | 7704 | CA | ASN | B | 2019 | −31.286 | −31.377 | 47.378 | 1.00 | 195.15 | C |
| ATOM | 7705 | C | ASN | B | 2019 | −31.814 | −31.297 | 45.961 | 1.00 | 195.83 | C |
| ATOM | 7706 | O | ASN | B | 2019 | −31.044 | −31.169 | 45.007 | 1.00 | 196.11 | O |
| ATOM | 7707 | CB | ASN | B | 2019 | −30.203 | −32.437 | 47.525 | 1.00 | 195.06 | C |
| ATOM | 7708 | CG | ASN | B | 2019 | −30.767 | −33.843 | 47.489 | 1.00 | 196.25 | C |
| ATOM | 7709 | OD1 | ASN | B | 2019 | −31.981 | −34.043 | 47.322 | 1.00 | 197.19 | O |
| ATOM | 7710 | ND2 | ASN | B | 2019 | −29.893 | −34.834 | 47.654 | 1.00 | 197.57 | N |
| ATOM | 7711 | N | LYS | B | 2020 | −33.141 | −31.376 | 45.838 | 1.00 | 196.65 | N |
| ATOM | 7712 | CA | LYS | B | 2020 | −33.830 | −30.995 | 44.606 | 1.00 | 197.65 | C |
| ATOM | 7713 | C | LYS | B | 2020 | −34.023 | −29.472 | 44.569 | 1.00 | 198.39 | C |
| ATOM | 7714 | O | LYS | B | 2020 | −34.550 | −28.915 | 43.606 | 1.00 | 198.72 | O |
| ATOM | 7715 | CB | LYS | B | 2020 | −35.158 | −31.745 | 44.445 | 1.00 | 197.47 | C |
| ATOM | 7716 | CG | LYS | B | 2020 | −34.994 | −33.194 | 43.991 | 1.00 | 197.40 | C |
| ATOM | 7717 | CD | LYS | B | 2020 | −36.199 | −33.683 | 43.198 | 1.00 | 197.14 | C |
| ATOM | 7718 | CE | LYS | B | 2020 | −35.890 | −34.993 | 42.477 | 1.00 | 197.01 | C |
| ATOM | 7719 | NZ | LYS | B | 2020 | −37.010 | −35.453 | 41.597 | 1.00 | 196.88 | N |
| ATOM | 7720 | N | CYS | B | 2021 | −33.575 | −28.815 | 45.634 | 1.00 | 199.60 | N |
| ATOM | 7721 | CA | CYS | B | 2021 | −33.498 | −27.366 | 45.721 | 1.00 | 199.69 | C |
| ATOM | 7722 | C | CYS | B | 2021 | −32.071 | −26.984 | 45.402 | 1.00 | 199.31 | C |
| ATOM | 7723 | O | CYS | B | 2021 | −31.141 | −27.369 | 46.110 | 1.00 | 198.93 | O |
| ATOM | 7724 | CB | CYS | B | 2021 | −33.833 | −26.920 | 47.158 | 1.00 | 200.46 | C |
| ATOM | 7725 | SG | CYS | B | 2021 | −34.040 | −25.092 | 47.557 | 1.00 | 202.26 | S |
| ATOM | 7726 | N | GLN | B | 2022 | −31.888 | −26.231 | 44.334 | 1.00 | 199.21 | N |
| ATOM | 7727 | CA | GLN | B | 2022 | −30.569 | −25.708 | 44.038 | 1.00 | 199.56 | C |
| ATOM | 7728 | C | GLN | B | 2022 | −30.677 | −24.671 | 42.925 | 1.00 | 199.39 | C |
| ATOM | 7729 | O | GLN | B | 2022 | −30.700 | −25.020 | 41.744 | 1.00 | 199.84 | O |
| ATOM | 7730 | CB | GLN | B | 2022 | −29.605 | −26.859 | 43.670 | 1.00 | 199.63 | C |
| ATOM | 7731 | CG | GLN | B | 2022 | −28.098 | −26.530 | 43.792 | 1.00 | 200.18 | C |
| ATOM | 7732 | CD | GLN | B | 2022 | −27.174 | −27.762 | 43.688 | 1.00 | 200.06 | C |
| ATOM | 7733 | OE1 | GLN | B | 2022 | −27.459 | −28.823 | 44.259 | 1.00 | 200.43 | O |
| ATOM | 7734 | NE2 | GLN | B | 2022 | −26.051 | −27.607 | 42.973 | 1.00 | 200.19 | N |
| ATOM | 7735 | N | THR | B | 2023 | −30.778 | −23.397 | 43.292 | 1.00 | 198.89 | N |
| ATOM | 7736 | CA | THR | B | 2023 | −30.784 | −22.349 | 42.282 | 1.00 | 198.43 | C |
| ATOM | 7737 | C | THR | B | 2023 | −29.524 | −21.512 | 42.447 | 1.00 | 198.00 | C |
| ATOM | 7738 | O | THR | B | 2023 | −28.945 | −21.473 | 43.528 | 1.00 | 197.83 | O |
| ATOM | 7739 | CB | THR | B | 2023 | −32.113 | −21.506 | 42.297 | 1.00 | 198.48 | C |
| ATOM | 7740 | OG1 | THR | B | 2023 | −32.392 | −20.972 | 40.991 | 1.00 | 198.52 | O |
| ATOM | 7741 | CG2 | THR | B | 2023 | −32.059 | −20.378 | 43.308 | 1.00 | 198.56 | C |
| ATOM | 7742 | N | PRO | B | 2024 | −29.052 | −20.896 | 41.359 | 1.00 | 197.78 | N |
| ATOM | 7743 | CA | PRO | B | 2024 | −28.048 | −19.865 | 41.514 | 1.00 | 197.81 | C |
| ATOM | 7744 | C | PRO | B | 2024 | −28.460 | −18.904 | 42.615 | 1.00 | 197.87 | C |
| ATOM | 7745 | O | PRO | B | 2024 | −29.574 | −18.383 | 42.591 | 1.00 | 197.93 | O |
| ATOM | 7746 | CB | PRO | B | 2024 | −28.081 | −19.150 | 40.162 | 1.00 | 197.93 | C |
| ATOM | 7747 | CG | PRO | B | 2024 | −28.494 | −20.184 | 39.199 | 1.00 | 197.93 | C |
| ATOM | 7748 | CD | PRO | B | 2024 | −29.376 | −21.147 | 39.944 | 1.00 | 197.78 | C |
| ATOM | 7749 | N | LEU | B | 2025 | −27.568 | −18.672 | 43.569 | 1.00 | 197.99 | N |
| ATOM | 7750 | CA | LEU | B | 2025 | −27.853 | −17.776 | 44.692 | 1.00 | 198.39 | C |
| ATOM | 7751 | C | LEU | B | 2025 | −27.916 | −16.284 | 44.356 | 1.00 | 198.75 | C |
| ATOM | 7752 | O | LEU | B | 2025 | −28.045 | −15.449 | 45.260 | 1.00 | 198.83 | O |
| ATOM | 7753 | CB | LEU | B | 2025 | −26.840 | −18.002 | 45.804 | 1.00 | 198.30 | C |
| ATOM | 7754 | CG | LEU | B | 2025 | −27.114 | −19.250 | 46.628 | 1.00 | 198.67 | C |
| ATOM | 7755 | CD1 | LEU | B | 2025 | −25.845 | −19.692 | 47.293 | 1.00 | 199.48 | C |
| ATOM | 7756 | CD2 | LEU | B | 2025 | −28.200 | −18.984 | 47.665 | 1.00 | 199.22 | C |
| ATOM | 7757 | N | GLY | B | 2026 | −27.823 | −15.961 | 43.064 | 1.00 | 199.20 | N |
| ATOM | 7758 | CA | GLY | B | 2026 | −27.839 | −14.578 | 42.570 | 1.00 | 199.47 | C |
| ATOM | 7759 | C | GLY | B | 2026 | −26.479 | −13.896 | 42.440 | 1.00 | 199.64 | C |
| ATOM | 7760 | O | GLY | B | 2026 | −26.027 | −13.214 | 43.371 | 1.00 | 199.57 | O |
| ATOM | 7761 | N | MET | B | 2027 | −25.823 | −14.084 | 41.293 | 1.00 | 199.71 | N |
| ATOM | 7762 | CA | MET | B | 2027 | −24.625 | −13.310 | 40.969 | 1.00 | 199.75 | C |
| ATOM | 7763 | C | MET | B | 2027 | −24.832 | −12.593 | 39.658 | 1.00 | 200.07 | C |
| ATOM | 7764 | O | MET | B | 2027 | −24.165 | −11.614 | 39.382 | 1.00 | 200.02 | O |
| ATOM | 7765 | CB | MET | B | 2027 | −23.354 | −14.162 | 40.949 | 1.00 | 199.60 | C |
| ATOM | 7766 | CG | MET | B | 2027 | −22.897 | −14.681 | 42.334 | 1.00 | 198.77 | C |
| ATOM | 7767 | SD | MET | B | 2027 | −21.545 | −13.798 | 43.158 | 1.00 | 196.36 | S |
| ATOM | 7768 | CE | MET | B | 2027 | −22.430 | −12.530 | 44.056 | 1.00 | 196.41 | C |
| ATOM | 7769 | N | ALA | B | 2028 | −25.761 | −13.078 | 38.849 | 1.00 | 200.63 | N |
| ATOM | 7770 | CA | ALA | B | 2028 | −26.324 | −12.224 | 37.829 | 1.00 | 201.46 | C |
| ATOM | 7771 | C | ALA | B | 2028 | −27.496 | −11.559 | 38.502 | 1.00 | 202.15 | C |
| ATOM | 7772 | O | ALA | B | 2028 | −27.570 | −10.342 | 38.573 | 1.00 | 201.87 | O |
| ATOM | 7773 | CB | ALA | B | 2028 | −26.776 | −13.019 | 36.648 | 1.00 | 201.43 | C |
| ATOM | 7774 | N | SER | B | 2029 | −28.382 | −12.387 | 39.043 | 1.00 | 203.55 | N |
| ATOM | 7775 | CA | SER | B | 2029 | −29.619 | −11.929 | 39.693 | 1.00 | 205.11 | C |
| ATOM | 7776 | C | SER | B | 2029 | −29.481 | −11.718 | 41.225 | 1.00 | 205.89 | C |
| ATOM | 7777 | O | SER | B | 2029 | −30.229 | −12.296 | 42.043 | 1.00 | 206.14 | O |
| ATOM | 7778 | CB | SER | B | 2029 | −30.780 | −12.894 | 39.368 | 1.00 | 205.22 | C |
| ATOM | 7779 | OG | SER | B | 2029 | −31.434 | −12.558 | 38.149 | 1.00 | 205.45 | O |
| ATOM | 7780 | N | GLY | B | 2030 | −28.535 | −10.874 | 41.615 | 1.00 | 206.50 | N |
| ATOM | 7781 | CA | GLY | B | 2030 | −28.237 | −10.752 | 43.021 | 1.00 | 207.06 | C |
| ATOM | 7782 | C | GLY | B | 2030 | −28.361 | −9.369 | 43.590 | 1.00 | 207.53 | C |
| ATOM | 7783 | O | GLY | B | 2030 | −29.395 | −9.016 | 44.157 | 1.00 | 207.55 | O |
| ATOM | 7784 | N | HIS | B | 2031 | −27.297 | −8.591 | 43.408 | 1.00 | 207.99 | N |
| ATOM | 7785 | CA | HIS | B | 2031 | −26.960 | −7.496 | 44.304 | 1.00 | 208.59 | C |
| ATOM | 7786 | C | HIS | B | 2031 | −25.851 | −6.662 | 43.695 | 1.00 | 209.19 | C |
| ATOM | 7787 | O | HIS | B | 2031 | −24.678 | −7.037 | 43.798 | 1.00 | 209.04 | O |
| ATOM | 7788 | CB | HIS | B | 2031 | −26.467 | −8.091 | 45.616 | 1.00 | 208.47 | C |
| ATOM | 7789 | CG | HIS | B | 2031 | −26.127 | −9.548 | 45.515 | 1.00 | 208.37 | C |
| ATOM | 7790 | ND1 | HIS | B | 2031 | −26.817 | −10.522 | 46.206 | 1.00 | 208.32 | N |
| ATOM | 7791 | CD2 | HIS | B | 2031 | −25.212 | −10.203 | 44.759 | 1.00 | 207.95 | C |
| ATOM | 7792 | CE1 | HIS | B | 2031 | −26.323 | −11.709 | 45.904 | 1.00 | 207.71 | C |
| ATOM | 7793 | NE2 | HIS | B | 2031 | −25.350 | −11.544 | 45.026 | 1.00 | 207.37 | N |
| ATOM | 7794 | N | ILE | B | 2032 | −26.244 | −5.520 | 43.106 | 1.00 | 210.19 | N |
| ATOM | 7795 | CA | ILE | B | 2032 | −25.427 | −4.665 | 42.177 | 1.00 | 210.81 | C |
| ATOM | 7796 | C | ILE | B | 2032 | −23.914 | −4.501 | 42.490 | 1.00 | 211.28 | C |
| ATOM | 7797 | O | ILE | B | 2032 | −23.060 | −4.660 | 41.597 | 1.00 | 211.28 | O |
| ATOM | 7798 | CB | ILE | B | 2032 | −26.123 | −3.271 | 41.874 | 1.00 | 210.74 | C |
| ATOM | 7799 | CG1 | ILE | B | 2032 | −27.504 | −3.477 | 41.222 | 1.00 | 210.69 | C |
| ATOM | 7800 | CG2 | ILE | B | 2032 | −25.242 | −2.387 | 40.981 | 1.00 | 210.49 | C |
| ATOM | 7801 | CD1 | ILE | B | 2032 | −28.175 | −2.198 | 40.697 | 1.00 | 210.79 | C |
| ATOM | 7802 | N | ARG | B | 2033 | −23.603 | −4.150 | 43.738 | 1.00 | 211.72 | N |
| ATOM | 7803 | CA | ARG | B | 2033 | −22.244 | −4.229 | 44.276 | 1.00 | 212.03 | C |
| ATOM | 7804 | C | ARG | B | 2033 | −22.384 | −4.595 | 45.752 | 1.00 | 212.20 | C |
| ATOM | 7805 | O | ARG | B | 2033 | −22.352 | −3.739 | 46.638 | 1.00 | 212.14 | O |
| ATOM | 7806 | CB | ARG | B | 2033 | −21.458 | −2.929 | 44.033 | 1.00 | 212.04 | C |
| ATOM | 7807 | CG | ARG | B | 2033 | −20.831 | −2.847 | 42.624 | 1.00 | 212.18 | C |
| ATOM | 7808 | CD | ARG | B | 2033 | −20.400 | −1.430 | 42.187 | 1.00 | 212.23 | C |
| ATOM | 7809 | NE | ARG | B | 2033 | −19.847 | −1.420 | 40.821 | 1.00 | 212.04 | N |
| ATOM | 7810 | CZ | ARG | B | 2033 | −19.316 | −0.358 | 40.208 | 1.00 | 211.67 | C |
| ATOM | 7811 | NH1 | ARG | B | 2033 | −19.247 | 0.821 | 40.817 | 1.00 | 211.61 | N |
| ATOM | 7812 | NH2 | ARG | B | 2033 | −18.847 | −0.477 | 38.973 | 1.00 | 211.43 | N |
| ATOM | 7813 | N | ASP | B | 2034 | −22.587 | −5.888 | 45.987 | 1.00 | 212.52 | N |
| ATOM | 7814 | CA | ASP | B | 2034 | −22.935 | −6.405 | 47.305 | 1.00 | 213.01 | C |
| ATOM | 7815 | C | ASP | B | 2034 | −21.791 | −6.201 | 48.305 | 1.00 | 213.35 | C |
| ATOM | 7816 | O | ASP | B | 2034 | −21.716 | −5.147 | 48.970 | 1.00 | 213.23 | O |
| ATOM | 7817 | CB | ASP | B | 2034 | −23.341 | −7.891 | 47.196 | 1.00 | 212.96 | C |
| ATOM | 7818 | CG | ASP | B | 2034 | −23.956 | −8.455 | 48.497 | 1.00 | 212.72 | C |
| ATOM | 7819 | OD1 | ASP | B | 2034 | −23.195 | −8.832 | 49.417 | 1.00 | 211.73 | O |
| ATOM | 7820 | OD2 | ASP | B | 2034 | −25.201 | −8.565 | 48.584 | 1.00 | 212.22 | O |
| ATOM | 7821 | N | PHE | B | 2035 | −20.921 | −7.216 | 48.388 | 1.00 | 213.66 | N |
| ATOM | 7822 | CA | PHE | B | 2035 | −19.820 | −7.310 | 49.354 | 1.00 | 213.85 | C |
| ATOM | 7823 | C | PHE | B | 2035 | −18.588 | −7.853 | 48.620 | 1.00 | 213.36 | C |
| ATOM | 7824 | O | PHE | B | 2035 | −18.721 | −8.516 | 47.584 | 1.00 | 213.01 | O |
| ATOM | 7825 | CB | PHE | B | 2035 | −20.205 | −8.226 | 50.547 | 1.00 | 214.47 | C |
| ATOM | 7826 | CG | PHE | B | 2035 | −20.854 | −7.493 | 51.752 | 1.00 | 215.64 | C |
| ATOM | 7827 | CD1 | PHE | B | 2035 | −21.955 | −6.625 | 51.590 | 1.00 | 216.36 | C |
| ATOM | 7828 | CD2 | PHE | B | 2035 | −20.382 | −7.714 | 53.064 | 1.00 | 216.40 | C |
| ATOM | 7829 | CE1 | PHE | B | 2035 | −22.551 | −5.970 | 52.706 | 1.00 | 215.68 | C |
| ATOM | 7830 | CE2 | PHE | B | 2035 | −20.977 | −7.063 | 54.190 | 1.00 | 215.48 | C |
| ATOM | 7831 | CZ | PHE | B | 2035 | −22.060 | −6.196 | 54.005 | 1.00 | 215.32 | C |
| ATOM | 7832 | N | GLN | B | 2036 | −17.401 | −7.578 | 49.163 | 1.00 | 213.01 | N |
| ATOM | 7833 | CA | GLN | B | 2036 | −16.141 | −7.863 | 48.464 | 1.00 | 212.86 | C |
| ATOM | 7834 | C | GLN | B | 2036 | −14.983 | −8.374 | 49.347 | 1.00 | 212.47 | C |
| ATOM | 7835 | O | GLN | B | 2036 | −14.726 | −9.571 | 49.402 | 1.00 | 212.20 | O |
| ATOM | 7836 | CB | GLN | B | 2036 | −15.698 | −6.640 | 47.626 | 1.00 | 213.03 | C |
| ATOM | 7837 | CG | GLN | B | 2036 | −15.899 | −5.241 | 48.289 | 1.00 | 213.34 | C |
| ATOM | 7838 | CD | GLN | B | 2036 | −14.937 | −4.149 | 47.766 | 1.00 | 213.19 | C |
| ATOM | 7839 | OE1 | GLN | B | 2036 | −14.479 | −4.192 | 46.617 | 1.00 | 213.41 | O |
| ATOM | 7840 | NE2 | GLN | B | 2036 | −14.636 | −3.167 | 48.622 | 1.00 | 213.00 | N |
| ATOM | 7841 | N | ILE | B | 2037 | −14.270 | −7.443 | 49.983 | 1.00 | 212.38 | N |
| ATOM | 7842 | CA | ILE | B | 2037 | −13.158 | −7.706 | 50.919 | 1.00 | 212.16 | C |
| ATOM | 7843 | C | ILE | B | 2037 | −12.991 | −6.523 | 51.926 | 1.00 | 212.56 | C |
| ATOM | 7844 | O | ILE | B | 2037 | −13.991 | −5.942 | 52.386 | 1.00 | 212.68 | O |
| ATOM | 7845 | CB | ILE | B | 2037 | −11.793 | −8.053 | 50.195 | 1.00 | 211.76 | C |
| ATOM | 7846 | CG1 | ILE | B | 2037 | −10.992 | −6.798 | 49.773 | 1.00 | 211.15 | C |
| ATOM | 7847 | CG2 | ILE | B | 2037 | −12.003 | −9.014 | 49.065 | 1.00 | 210.96 | C |
| ATOM | 7848 | CD1 | ILE | B | 2037 | −11.522 | −5.985 | 48.598 | 1.00 | 210.57 | C |
| ATOM | 7849 | N | THR | B | 2038 | −11.735 | −6.192 | 52.267 | 1.00 | 212.62 | N |
| ATOM | 7850 | CA | THR | B | 2038 | −11.368 | −5.046 | 53.135 | 1.00 | 212.45 | C |
| ATOM | 7851 | C | THR | B | 2038 | −9.871 | −4.630 | 53.011 | 1.00 | 212.43 | C |
| ATOM | 7852 | O | THR | B | 2038 | −9.428 | −3.663 | 53.648 | 1.00 | 212.55 | O |
| ATOM | 7853 | CB | THR | B | 2038 | −11.789 | −5.260 | 54.632 | 1.00 | 212.39 | C |
| ATOM | 7854 | OG1 | THR | B | 2038 | −11.253 | −4.208 | 55.444 | 1.00 | 212.36 | O |
| ATOM | 7855 | CG2 | THR | B | 2038 | −11.311 | −6.607 | 55.172 | 1.00 | 212.34 | C |
| ATOM | 7856 | N | ALA | B | 2039 | −9.114 | −5.376 | 52.195 | 1.00 | 212.21 | N |
| ATOM | 7857 | CA | ALA | B | 2039 | −7.726 | −5.052 | 51.778 | 1.00 | 211.80 | C |
| ATOM | 7858 | C | ALA | B | 2039 | −7.240 | −6.086 | 50.745 | 1.00 | 211.43 | C |
| ATOM | 7859 | O | ALA | B | 2039 | −6.542 | −5.748 | 49.781 | 1.00 | 211.52 | O |
| ATOM | 7860 | CB | ALA | B | 2039 | −6.750 | −4.961 | 52.986 | 1.00 | 211.69 | C |
| ATOM | 7861 | N | SER | B | 2040 | −7.626 | −7.344 | 50.959 | 1.00 | 210.80 | N |
| ATOM | 7862 | CA | SER | B | 2040 | −7.322 | −8.429 | 50.053 | 1.00 | 210.11 | C |
| ATOM | 7863 | C | SER | B | 2040 | −8.090 | −8.148 | 48.797 | 1.00 | 210.01 | C |
| ATOM | 7864 | O | SER | B | 2040 | −9.288 | −8.389 | 48.756 | 1.00 | 209.96 | O |
| ATOM | 7865 | CB | SER | B | 2040 | −7.832 | −9.743 | 50.631 | 1.00 | 210.05 | C |
| ATOM | 7866 | OG | SER | B | 2040 | −9.250 | −9.723 | 50.725 | 1.00 | 209.08 | O |
| ATOM | 7867 | N | GLY | B | 2041 | −7.418 | −7.612 | 47.784 | 1.00 | 209.84 | N |
| ATOM | 7868 | CA | GLY | B | 2041 | −8.074 | −7.370 | 46.501 | 1.00 | 209.48 | C |
| ATOM | 7869 | C | GLY | B | 2041 | −7.922 | −5.965 | 45.970 | 1.00 | 209.11 | C |
| ATOM | 7870 | O | GLY | B | 2041 | −8.475 | −5.017 | 46.528 | 1.00 | 209.12 | O |
| ATOM | 7871 | N | GLN | B | 2042 | −7.175 | −5.849 | 44.879 | 1.00 | 208.75 | N |
| ATOM | 7872 | CA | GLN | B | 2042 | −6.891 | −4.574 | 44.251 | 1.00 | 208.53 | C |
| ATOM | 7873 | C | GLN | B | 2042 | −6.161 | −4.784 | 42.933 | 1.00 | 208.33 | C |
| ATOM | 7874 | O | GLN | B | 2042 | −5.235 | −5.601 | 42.839 | 1.00 | 208.31 | O |
| ATOM | 7875 | CB | GLN | B | 2042 | −6.075 | −3.671 | 45.197 | 1.00 | 208.61 | C |
| ATOM | 7876 | CG | GLN | B | 2042 | −5.147 | −2.622 | 44.532 | 1.00 | 208.93 | C |
| ATOM | 7877 | CD | GLN | B | 2042 | −5.871 | −1.401 | 43.963 | 1.00 | 209.03 | C |
| ATOM | 7878 | OE1 | GLN | B | 2042 | −6.791 | −0.861 | 44.582 | 1.00 | 209.08 | O |
| ATOM | 7879 | NE2 | GLN | B | 2042 | −5.434 | −0.950 | 42.785 | 1.00 | 208.73 | N |
| ATOM | 7880 | N | TYR | B | 2043 | −6.608 | −4.047 | 41.919 | 1.00 | 208.01 | N |
| ATOM | 7881 | CA | TYR | B | 2043 | −5.879 | −3.883 | 40.675 | 1.00 | 207.68 | C |
| ATOM | 7882 | C | TYR | B | 2043 | −6.705 | −2.984 | 39.773 | 1.00 | 207.64 | C |
| ATOM | 7883 | O | TYR | B | 2043 | −7.280 | −3.470 | 38.814 | 1.00 | 207.60 | O |
| ATOM | 7884 | CB | TYR | B | 2043 | −5.645 | −5.240 | 40.000 | 1.00 | 207.48 | C |
| ATOM | 7885 | CG | TYR | B | 2043 | −4.430 | −5.312 | 39.098 | 1.00 | 207.52 | C |
| ATOM | 7886 | CD1 | TYR | B | 2043 | −4.437 | −4.720 | 37.828 | 1.00 | 207.88 | C |
| ATOM | 7887 | CD2 | TYR | B | 2043 | −3.276 | −5.993 | 39.498 | 1.00 | 207.49 | C |
| ATOM | 7888 | CE1 | TYR | B | 2043 | −3.315 | −4.784 | 36.977 | 1.00 | 207.64 | C |
| ATOM | 7889 | CE2 | TYR | B | 2043 | −2.148 | −6.071 | 38.652 | 1.00 | 207.62 | C |
| ATOM | 7890 | CZ | TYR | B | 2043 | −2.178 | −5.461 | 37.393 | 1.00 | 207.54 | C |
| ATOM | 7891 | OH | TYR | B | 2043 | −1.085 | −5.524 | 36.551 | 1.00 | 207.29 | O |
| ATOM | 7892 | N | GLY | B | 2044 | −6.774 | −1.686 | 40.093 | 1.00 | 207.69 | N |
| ATOM | 7893 | CA | GLY | B | 2044 | −7.499 | −0.685 | 39.283 | 1.00 | 207.81 | C |
| ATOM | 7894 | C | GLY | B | 2044 | −8.882 | −1.133 | 38.838 | 1.00 | 207.96 | C |
| ATOM | 7895 | O | GLY | B | 2044 | −9.889 | −0.657 | 39.356 | 1.00 | 207.98 | O |
| ATOM | 7896 | N | GLN | B | 2045 | −8.907 | −2.020 | 37.837 | 1.00 | 208.16 | N |
| ATOM | 7897 | CA | GLN | B | 2045 | −10.043 | −2.900 | 37.510 | 1.00 | 208.19 | C |
| ATOM | 7898 | C | GLN | B | 2045 | −10.260 | −3.808 | 38.716 | 1.00 | 208.46 | C |
| ATOM | 7899 | O | GLN | B | 2045 | −9.653 | −4.875 | 38.842 | 1.00 | 208.33 | O |
| ATOM | 7900 | CB | GLN | B | 2045 | −9.710 | −3.731 | 36.254 | 1.00 | 208.38 | C |
| ATOM | 7901 | CG | GLN | B | 2045 | −10.372 | −5.118 | 36.163 | 1.00 | 208.01 | C |
| ATOM | 7902 | CD | GLN | B | 2045 | −9.541 | −6.134 | 35.387 | 1.00 | 207.40 | C |
| ATOM | 7903 | OE1 | GLN | B | 2045 | −8.490 | −6.596 | 35.844 | 1.00 | 205.21 | O |
| ATOM | 7904 | NE2 | GLN | B | 2045 | −10.027 | −6.501 | 34.214 | 1.00 | 206.32 | N |
| ATOM | 7905 | N | TRP | B | 2046 | −11.129 | −3.371 | 39.611 | 1.00 | 208.86 | N |
| ATOM | 7906 | CA | TRP | B | 2046 | −11.058 | −3.835 | 40.987 | 1.00 | 209.18 | C |
| ATOM | 7907 | C | TRP | B | 2046 | −11.566 | −5.247 | 41.216 | 1.00 | 208.72 | C |
| ATOM | 7908 | O | TRP | B | 2046 | −11.755 | −6.046 | 40.284 | 1.00 | 208.26 | O |
| ATOM | 7909 | CB | TRP | B | 2046 | −11.754 | −2.832 | 41.940 | 1.00 | 209.91 | C |
| ATOM | 7910 | CG | TRP | B | 2046 | −11.062 | −2.635 | 43.299 | 1.00 | 210.84 | C |
| ATOM | 7911 | CD1 | TRP | B | 2046 | −9.733 | −2.820 | 43.576 | 1.00 | 211.30 | C |
| ATOM | 7912 | CD2 | TRP | B | 2046 | −11.667 | −2.181 | 44.539 | 1.00 | 212.11 | C |
| ATOM | 7913 | NE1 | TRP | B | 2046 | −9.477 | −2.532 | 44.900 | 1.00 | 212.06 | N |
| ATOM | 7914 | CE2 | TRP | B | 2046 | −10.639 | −2.135 | 45.515 | 1.00 | 212.29 | C |
| ATOM | 7915 | CE3 | TRP | B | 2046 | −12.975 | −1.815 | 44.919 | 1.00 | 212.15 | C |
| ATOM | 7916 | CZ2 | TRP | B | 2046 | −10.877 | −1.736 | 46.854 | 1.00 | 211.45 | C |
| ATOM | 7917 | CZ3 | TRP | B | 2046 | −13.207 | −1.413 | 46.255 | 1.00 | 211.35 | C |
| ATOM | 7918 | CH2 | TRP | B | 2046 | −12.161 | −1.384 | 47.198 | 1.00 | 211.00 | C |
| ATOM | 7919 | N | ALA | B | 2047 | −11.744 | −5.512 | 42.506 | 1.00 | 208.37 | N |
| ATOM | 7920 | CA | ALA | B | 2047 | −12.257 | −6.735 | 43.076 | 1.00 | 208.02 | C |
| ATOM | 7921 | C | ALA | B | 2047 | −13.646 | −7.064 | 42.543 | 1.00 | 207.62 | C |
| ATOM | 7922 | O | ALA | B | 2047 | −14.037 | −6.582 | 41.481 | 1.00 | 207.67 | O |
| ATOM | 7923 | CB | ALA | B | 2047 | −12.294 | −6.572 | 44.608 | 1.00 | 208.01 | C |
| ATOM | 7924 | N | PRO | B | 2048 | −14.384 | −7.929 | 43.257 | 1.00 | 207.15 | N |
| ATOM | 7925 | CA | PRO | B | 2048 | −15.811 | −7.841 | 43.160 | 1.00 | 206.67 | C |
| ATOM | 7926 | C | PRO | B | 2048 | −16.199 | −6.403 | 43.355 | 1.00 | 206.29 | C |
| ATOM | 7927 | O | PRO | B | 2048 | −15.416 | −5.599 | 43.882 | 1.00 | 206.18 | O |
| ATOM | 7928 | CB | PRO | B | 2048 | −16.284 | −8.655 | 44.350 | 1.00 | 206.83 | C |
| ATOM | 7929 | CG | PRO | B | 2048 | −15.265 | −9.694 | 44.488 | 1.00 | 207.33 | C |
| ATOM | 7930 | CD | PRO | B | 2048 | −13.970 | −9.044 | 44.123 | 1.00 | 207.27 | C |
| ATOM | 7931 | N | LYS | B | 2049 | −17.442 | −6.127 | 42.994 | 1.00 | 205.91 | N |
| ATOM | 7932 | CA | LYS | B | 2049 | −17.893 | −4.848 | 42.478 | 1.00 | 205.53 | C |
| ATOM | 7933 | C | LYS | B | 2049 | −18.446 | −5.381 | 41.168 | 1.00 | 204.96 | C |
| ATOM | 7934 | O | LYS | B | 2049 | −19.510 | −4.952 | 40.695 | 1.00 | 204.89 | O |
| ATOM | 7935 | CB | LYS | B | 2049 | −16.711 | −3.882 | 42.217 | 1.00 | 205.66 | C |
| ATOM | 7936 | CG | LYS | B | 2049 | −17.057 | −2.392 | 42.044 | 1.00 | 205.66 | C |
| ATOM | 7937 | CD | LYS | B | 2049 | −15.818 | −1.551 | 41.668 | 1.00 | 205.84 | C |
| ATOM | 7938 | CE | LYS | B | 2049 | −16.060 | −0.011 | 41.713 | 1.00 | 206.18 | C |
| ATOM | 7939 | NZ | LYS | B | 2049 | −16.139 | 0.715 | 40.388 | 1.00 | 205.55 | N |
| ATOM | 7940 | N | LEU | B | 2050 | −17.725 | −6.387 | 40.647 | 1.00 | 204.16 | N |
| ATOM | 7941 | CA | LEU | B | 2050 | −17.861 | −6.884 | 39.278 | 1.00 | 203.24 | C |
| ATOM | 7942 | C | LEU | B | 2050 | −17.183 | −8.245 | 39.048 | 1.00 | 202.39 | C |
| ATOM | 7943 | O | LEU | B | 2050 | −16.704 | −8.510 | 37.947 | 1.00 | 202.48 | O |
| ATOM | 7944 | CB | LEU | B | 2050 | −17.360 | −5.817 | 38.257 | 1.00 | 203.34 | C |
| ATOM | 7945 | CG | LEU | B | 2050 | −15.925 | −5.447 | 37.792 | 1.00 | 203.29 | C |
| ATOM | 7946 | CD1 | LEU | B | 2050 | −15.889 | −4.003 | 37.254 | 1.00 | 203.33 | C |
| ATOM | 7947 | CD2 | LEU | B | 2050 | −14.801 | −5.647 | 38.814 | 1.00 | 203.12 | C |
| ATOM | 7948 | N | ALA | B | 2051 | −17.157 | −9.112 | 40.059 | 1.00 | 201.23 | N |
| ATOM | 7949 | CA | ALA | B | 2051 | −16.478 | −10.413 | 39.904 | 1.00 | 200.41 | C |
| ATOM | 7950 | C | ALA | B | 2051 | −17.399 | −11.616 | 39.665 | 1.00 | 199.75 | C |
| ATOM | 7951 | O | ALA | B | 2051 | −17.119 | −12.729 | 40.109 | 1.00 | 199.29 | O |
| ATOM | 7952 | CB | ALA | B | 2051 | −15.550 | −10.675 | 41.072 | 1.00 | 200.49 | C |
| ATOM | 7953 | N | ARG | B | 2052 | −18.481 | −11.386 | 38.937 | 1.00 | 199.25 | N |
| ATOM | 7954 | CA | ARG | B | 2052 | −19.519 | −12.389 | 38.757 | 1.00 | 198.91 | C |
| ATOM | 7955 | C | ARG | B | 2052 | −19.214 | −13.284 | 37.589 | 1.00 | 199.04 | C |
| ATOM | 7956 | O | ARG | B | 2052 | −18.371 | −12.966 | 36.753 | 1.00 | 199.14 | O |
| ATOM | 7957 | CB | ARG | B | 2052 | −20.868 | −11.723 | 38.498 | 1.00 | 198.73 | C |
| ATOM | 7958 | CG | ARG | B | 2052 | −21.287 | −10.709 | 39.537 | 1.00 | 197.85 | C |
| ATOM | 7959 | CD | ARG | B | 2052 | −20.540 | −9.386 | 39.398 | 1.00 | 195.69 | C |
| ATOM | 7960 | NE | ARG | B | 2052 | −21.085 | −8.304 | 40.220 | 1.00 | 194.54 | N |
| ATOM | 7961 | CZ | ARG | B | 2052 | −22.255 | −8.320 | 40.861 | 1.00 | 194.27 | C |
| ATOM | 7962 | NH1 | ARG | B | 2052 | −23.070 | −9.371 | 40.819 | 1.00 | 193.69 | N |
| ATOM | 7963 | NH2 | ARG | B | 2052 | −22.613 | −7.258 | 41.559 | 1.00 | 194.93 | N |
| ATOM | 7964 | N | LEU | B | 2053 | −19.939 | −14.394 | 37.524 | 1.00 | 199.14 | N |
| ATOM | 7965 | CA | LEU | B | 2053 | −19.839 | −15.327 | 36.413 | 1.00 | 199.31 | C |
| ATOM | 7966 | C | LEU | B | 2053 | −20.364 | −14.686 | 35.133 | 1.00 | 199.46 | C |
| ATOM | 7967 | O | LEU | B | 2053 | −21.335 | −13.925 | 35.164 | 1.00 | 199.47 | O |
| ATOM | 7968 | CB | LEU | B | 2053 | −20.627 | −16.603 | 36.736 | 1.00 | 199.33 | C |
| ATOM | 7969 | CG | LEU | B | 2053 | −20.452 | −17.887 | 35.914 | 1.00 | 199.32 | C |
| ATOM | 7970 | CD1 | LEU | B | 2053 | −19.058 | −18.467 | 36.071 | 1.00 | 199.34 | C |
| ATOM | 7971 | CD2 | LEU | B | 2053 | −21.513 | −18.925 | 36.293 | 1.00 | 199.26 | C |
| ATOM | 7972 | N | HIS | B | 2054 | −19.688 | −14.982 | 34.023 | 1.00 | 199.70 | N |
| ATOM | 7973 | CA | HIS | B | 2054 | −20.111 | −14.600 | 32.661 | 1.00 | 199.99 | C |
| ATOM | 7974 | C | HIS | B | 2054 | −20.012 | −13.121 | 32.319 | 1.00 | 199.94 | C |
| ATOM | 7975 | O | HIS | B | 2054 | −20.291 | −12.731 | 31.176 | 1.00 | 199.88 | O |
| ATOM | 7976 | CB | HIS | B | 2054 | −21.531 | −15.095 | 32.348 | 1.00 | 200.21 | C |
| ATOM | 7977 | CG | HIS | B | 2054 | −21.693 | −16.578 | 32.448 | 1.00 | 200.91 | C |
| ATOM | 7978 | ND1 | HIS | B | 2054 | −20.929 | −17.463 | 31.716 | 1.00 | 201.42 | N |
| ATOM | 7979 | CD2 | HIS | B | 2054 | −22.545 | −17.332 | 33.182 | 1.00 | 201.41 | C |
| ATOM | 7980 | CE1 | HIS | B | 2054 | −21.294 | −18.699 | 32.008 | 1.00 | 201.92 | C |
| ATOM | 7981 | NE2 | HIS | B | 2054 | −22.273 | −18.647 | 32.895 | 1.00 | 201.94 | N |
| ATOM | 7982 | N | TYR | B | 2055 | −19.616 | −12.306 | 33.298 | 1.00 | 199.94 | N |
| ATOM | 7983 | CA | TYR | B | 2055 | −19.545 | −10.852 | 33.119 | 1.00 | 199.93 | C |
| ATOM | 7984 | C | TYR | B | 2055 | −18.630 | −10.448 | 31.935 | 1.00 | 199.99 | C |
| ATOM | 7985 | O | TYR | B | 2055 | −17.400 | −10.557 | 32.005 | 1.00 | 200.07 | O |
| ATOM | 7986 | CB | TYR | B | 2055 | −19.180 | −10.149 | 34.448 | 1.00 | 199.69 | C |
| ATOM | 7987 | CG | TYR | B | 2055 | −19.228 | −8.625 | 34.417 | 1.00 | 199.36 | C |
| ATOM | 7988 | CD1 | TYR | B | 2055 | −20.260 | −7.935 | 33.762 | 1.00 | 198.89 | C |
| ATOM | 7989 | CD2 | TYR | B | 2055 | −18.246 | −7.876 | 35.062 | 1.00 | 198.80 | C |
| ATOM | 7990 | CE1 | TYR | B | 2055 | −20.290 | −6.547 | 33.739 | 1.00 | 198.74 | C |
| ATOM | 7991 | CE2 | TYR | B | 2055 | −18.270 | −6.494 | 35.046 | 1.00 | 198.58 | C |
| ATOM | 7992 | CZ | TYR | B | 2055 | −19.287 | −5.835 | 34.388 | 1.00 | 199.05 | C |
| ATOM | 7993 | OH | TYR | B | 2055 | −19.283 | −4.459 | 34.389 | 1.00 | 199.69 | O |
| ATOM | 7994 | N | SER | B | 2056 | −19.266 | −10.013 | 30.845 | 1.00 | 199.87 | N |
| ATOM | 7995 | CA | SER | B | 2056 | −18.576 | −9.617 | 29.615 | 1.00 | 199.66 | C |
| ATOM | 7996 | C | SER | B | 2056 | −17.889 | −8.262 | 29.746 | 1.00 | 199.46 | C |
| ATOM | 7997 | O | SER | B | 2056 | −18.274 | −7.433 | 30.576 | 1.00 | 199.48 | O |
| ATOM | 7998 | CB | SER | B | 2056 | −19.564 | −9.591 | 28.438 | 1.00 | 199.79 | C |
| ATOM | 7999 | OG | SER | B | 2056 | −19.077 | −8.827 | 27.337 | 1.00 | 199.82 | O |
| ATOM | 8000 | N | GLY | B | 2057 | −16.876 | −8.053 | 28.906 | 1.00 | 199.19 | N |
| ATOM | 8001 | CA | GLY | B | 2057 | −16.142 | −6.791 | 28.836 | 1.00 | 198.75 | C |
| ATOM | 8002 | C | GLY | B | 2057 | −14.645 | −6.982 | 28.973 | 1.00 | 198.36 | C |
| ATOM | 8003 | O | GLY | B | 2057 | −14.158 | −8.109 | 29.067 | 1.00 | 198.43 | O |
| ATOM | 8004 | N | SER | B | 2058 | −13.915 | −5.874 | 28.974 | 1.00 | 197.90 | N |
| ATOM | 8005 | CA | SER | B | 2058 | −12.486 | −5.902 | 29.243 | 1.00 | 197.47 | C |
| ATOM | 8006 | C | SER | B | 2058 | −12.190 | −5.654 | 30.737 | 1.00 | 197.31 | C |
| ATOM | 8007 | O | SER | B | 2058 | −11.281 | −6.269 | 31.295 | 1.00 | 197.40 | O |
| ATOM | 8008 | CB | SER | B | 2058 | −11.772 | −4.897 | 28.347 | 1.00 | 197.41 | C |
| ATOM | 8009 | OG | SER | B | 2058 | −12.631 | −3.808 | 28.052 | 1.00 | 197.29 | O |
| ATOM | 8010 | N | ILE | B | 2059 | −12.975 | −4.774 | 31.373 | 1.00 | 196.89 | N |
| ATOM | 8011 | CA | ILE | B | 2059 | −12.851 | −4.432 | 32.806 | 1.00 | 196.19 | C |
| ATOM | 8012 | C | ILE | B | 2059 | −13.581 | −5.443 | 33.708 | 1.00 | 195.91 | C |
| ATOM | 8013 | O | ILE | B | 2059 | −14.337 | −5.032 | 34.587 | 1.00 | 196.02 | O |
| ATOM | 8014 | CB | ILE | B | 2059 | −13.424 | −2.991 | 33.116 | 1.00 | 196.16 | C |
| ATOM | 8015 | CG1 | ILE | B | 2059 | −14.946 | −2.909 | 32.843 | 1.00 | 195.80 | C |
| ATOM | 8016 | CG2 | ILE | B | 2059 | −12.657 | −1.909 | 32.339 | 1.00 | 195.88 | C |
| ATOM | 8017 | CD1 | ILE | B | 2059 | −15.764 | −2.155 | 33.905 | 1.00 | 194.55 | C |
| ATOM | 8018 | N | ASN | B | 2060 | −13.349 | −6.746 | 33.522 | 1.00 | 195.46 | N |
| ATOM | 8019 | CA | ASN | B | 2060 | −14.288 | −7.761 | 34.055 | 1.00 | 195.10 | C |
| ATOM | 8020 | C | ASN | B | 2060 | −13.790 | −8.879 | 34.994 | 1.00 | 194.98 | C |
| ATOM | 8021 | O | ASN | B | 2060 | −13.156 | −9.831 | 34.548 | 1.00 | 194.98 | O |
| ATOM | 8022 | CB | ASN | B | 2060 | −15.076 | −8.385 | 32.896 | 1.00 | 194.97 | C |
| ATOM | 8023 | CG | ASN | B | 2060 | −14.208 | −9.218 | 31.980 | 1.00 | 194.37 | C |
| ATOM | 8024 | OD1 | ASN | B | 2060 | −13.051 | −8.889 | 31.727 | 1.00 | 193.91 | O |
| ATOM | 8025 | ND2 | ASN | B | 2060 | −14.764 | −10.307 | 31.479 | 1.00 | 193.94 | N |
| ATOM | 8026 | N | ALA | B | 2061 | −14.130 | −8.763 | 36.281 | 1.00 | 194.85 | N |
| ATOM | 8027 | CA | ALA | B | 2061 | −13.863 | −9.782 | 37.326 | 1.00 | 194.88 | C |
| ATOM | 8028 | C | ALA | B | 2061 | −12.867 | −9.346 | 38.427 | 1.00 | 195.02 | C |
| ATOM | 8029 | O | ALA | B | 2061 | −12.497 | −8.158 | 38.512 | 1.00 | 195.12 | O |
| ATOM | 8030 | CB | ALA | B | 2061 | −13.480 | −11.140 | 36.725 | 1.00 | 194.67 | C |
| ATOM | 8031 | N | TRP | B | 2062 | −12.467 | −10.294 | 39.285 | 1.00 | 194.92 | N |
| ATOM | 8032 | CA | TRP | B | 2062 | −11.539 | −9.991 | 40.385 | 1.00 | 194.72 | C |
| ATOM | 8033 | C | TRP | B | 2062 | −10.088 | −10.245 | 39.976 | 1.00 | 194.98 | C |
| ATOM | 8034 | O | TRP | B | 2062 | −9.808 | −11.079 | 39.111 | 1.00 | 194.68 | O |
| ATOM | 8035 | CB | TRP | B | 2062 | −11.918 | −10.714 | 41.696 | 1.00 | 194.24 | C |
| ATOM | 8036 | CG | TRP | B | 2062 | −11.045 | −10.324 | 42.878 | 1.00 | 193.59 | C |
| ATOM | 8037 | CD1 | TRP | B | 2062 | −10.680 | −9.064 | 43.250 | 1.00 | 193.21 | C |
| ATOM | 8038 | CD2 | TRP | B | 2062 | −10.426 | −11.204 | 43.814 | 1.00 | 192.81 | C |
| ATOM | 8039 | NE1 | TRP | B | 2062 | −9.878 | −9.099 | 44.358 | 1.00 | 192.39 | N |
| ATOM | 8040 | CE2 | TRP | B | 2062 | −9.700 | −10.404 | 44.724 | 1.00 | 192.38 | C |
| ATOM | 8041 | CE3 | TRP | B | 2062 | −10.407 | −12.592 | 43.970 | 1.00 | 192.96 | C |
| ATOM | 8042 | CZ2 | TRP | B | 2062 | −8.969 | −10.942 | 45.772 | 1.00 | 192.98 | C |
| ATOM | 8043 | CZ3 | TRP | B | 2062 | −9.676 | −13.129 | 45.011 | 1.00 | 193.37 | C |
| ATOM | 8044 | CH2 | TRP | B | 2062 | −8.967 | −12.304 | 45.903 | 1.00 | 193.51 | C |
| ATOM | 8045 | N | SER | B | 2063 | −9.178 | −9.523 | 40.627 | 1.00 | 195.57 | N |
| ATOM | 8046 | CA | SER | B | 2063 | −7.856 | −9.244 | 40.085 | 1.00 | 196.26 | C |
| ATOM | 8047 | C | SER | B | 2063 | −6.839 | −8.767 | 41.141 | 1.00 | 196.59 | C |
| ATOM | 8048 | O | SER | B | 2063 | −6.792 | −7.573 | 41.453 | 1.00 | 196.79 | O |
| ATOM | 8049 | CB | SER | B | 2063 | −8.020 | −8.159 | 39.005 | 1.00 | 196.49 | C |
| ATOM | 8050 | OG | SER | B | 2063 | −9.094 | −7.264 | 39.320 | 1.00 | 196.56 | O |
| ATOM | 8051 | N | THR | B | 2064 | −6.026 | −9.674 | 41.689 | 1.00 | 196.87 | N |
| ATOM | 8052 | CA | THR | B | 2064 | −5.005 | −9.263 | 42.670 | 1.00 | 197.28 | C |
| ATOM | 8053 | C | THR | B | 2064 | −3.755 | −10.110 | 42.638 | 1.00 | 197.33 | C |
| ATOM | 8054 | O | THR | B | 2064 | −3.814 | −11.311 | 42.908 | 1.00 | 197.31 | O |
| ATOM | 8055 | CB | THR | B | 2064 | −5.511 | −9.310 | 44.134 | 1.00 | 197.42 | C |
| ATOM | 8056 | OG1 | THR | B | 2064 | −6.935 | −9.150 | 44.179 | 1.00 | 197.78 | O |
| ATOM | 8057 | CG2 | THR | B | 2064 | −4.813 | −8.225 | 44.981 | 1.00 | 197.50 | C |
| ATOM | 8058 | N | LYS | B | 2065 | −2.620 | −9.481 | 42.344 | 1.00 | 197.47 | N |
| ATOM | 8059 | CA | LYS | B | 2065 | −1.345 | −10.173 | 42.456 | 1.00 | 197.70 | C |
| ATOM | 8060 | C | LYS | B | 2065 | −0.945 | −10.148 | 43.912 | 1.00 | 197.93 | C |
| ATOM | 8061 | O | LYS | B | 2065 | −0.123 | −9.341 | 44.332 | 1.00 | 198.02 | O |
| ATOM | 8062 | CB | LYS | B | 2065 | −0.270 | −9.559 | 41.561 | 1.00 | 197.62 | C |
| ATOM | 8063 | CG | LYS | B | 2065 | −0.283 | −8.059 | 41.497 | 1.00 | 197.64 | C |
| ATOM | 8064 | CD | LYS | B | 2065 | 0.737 | −7.591 | 40.498 | 1.00 | 198.05 | C |
| ATOM | 8065 | CE | LYS | B | 2065 | 0.966 | −6.102 | 40.631 | 1.00 | 198.47 | C |
| ATOM | 8066 | NZ | LYS | B | 2065 | 1.852 | −5.583 | 39.554 | 1.00 | 198.81 | N |
| ATOM | 8067 | N | GLU | B | 2066 | −1.558 | −11.037 | 44.682 | 1.00 | 198.32 | N |
| ATOM | 8068 | CA | GLU | B | 2066 | −1.396 | −11.042 | 46.126 | 1.00 | 198.77 | C |
| ATOM | 8069 | C | GLU | B | 2066 | −1.276 | −12.469 | 46.682 | 1.00 | 198.87 | C |
| ATOM | 8070 | O | GLU | B | 2066 | −2.255 | −13.231 | 46.666 | 1.00 | 199.06 | O |
| ATOM | 8071 | CB | GLU | B | 2066 | −2.553 | −10.274 | 46.781 | 1.00 | 198.82 | C |
| ATOM | 8072 | CG | GLU | B | 2066 | −2.241 | −9.700 | 48.145 | 1.00 | 199.72 | C |
| ATOM | 8073 | CD | GLU | B | 2066 | −0.887 | −9.007 | 48.191 | 1.00 | 201.15 | C |
| ATOM | 8074 | OE1 | GLU | B | 2066 | −0.660 | −8.064 | 47.400 | 1.00 | 201.58 | O |
| ATOM | 8075 | OE2 | GLU | B | 2066 | −0.045 | −9.409 | 49.021 | 1.00 | 201.99 | O |
| ATOM | 8076 | N | PRO | B | 2067 | −0.066 | −12.842 | 47.159 | 1.00 | 198.88 | N |
| ATOM | 8077 | CA | PRO | B | 2067 | 0.188 | −14.171 | 47.739 | 1.00 | 198.81 | C |
| ATOM | 8078 | C | PRO | B | 2067 | −0.782 | −14.518 | 48.863 | 1.00 | 198.74 | C |
| ATOM | 8079 | O | PRO | B | 2067 | −1.087 | −15.689 | 49.076 | 1.00 | 198.61 | O |
| ATOM | 8080 | CB | PRO | B | 2067 | 1.613 | −14.046 | 48.283 | 1.00 | 198.81 | C |
| ATOM | 8081 | CG | PRO | B | 2067 | 2.250 | −13.005 | 47.416 | 1.00 | 198.77 | C |
| ATOM | 8082 | CD | PRO | B | 2067 | 1.156 | −12.011 | 47.149 | 1.00 | 198.82 | C |
| ATOM | 8083 | N | PHE | B | 2068 | −1.254 | −13.485 | 49.557 | 1.00 | 198.78 | N |
| ATOM | 8084 | CA | PHE | B | 2068 | −2.239 | −13.607 | 50.624 | 1.00 | 198.93 | C |
| ATOM | 8085 | C | PHE | B | 2068 | −3.528 | −12.880 | 50.221 | 1.00 | 198.71 | C |
| ATOM | 8086 | O | PHE | B | 2068 | −3.630 | −11.659 | 50.366 | 1.00 | 198.80 | O |
| ATOM | 8087 | CB | PHE | B | 2068 | −1.671 | −13.004 | 51.916 | 1.00 | 199.20 | C |
| ATOM | 8088 | CG | PHE | B | 2068 | −0.813 | −13.955 | 52.727 | 1.00 | 199.82 | C |
| ATOM | 8089 | CD1 | PHE | B | 2068 | 0.105 | −14.814 | 52.111 | 1.00 | 200.13 | C |
| ATOM | 8090 | CD2 | PHE | B | 2068 | −0.907 | −13.967 | 54.124 | 1.00 | 200.32 | C |
| ATOM | 8091 | CE1 | PHE | B | 2068 | 0.896 | −15.684 | 52.872 | 1.00 | 200.34 | C |
| ATOM | 8092 | CE2 | PHE | B | 2068 | −0.118 | −14.829 | 54.896 | 1.00 | 200.37 | C |
| ATOM | 8093 | CZ | PHE | B | 2068 | 0.786 | −15.688 | 54.268 | 1.00 | 200.23 | C |
| ATOM | 8094 | N | SER | B | 2069 | −4.508 | −13.622 | 49.716 | 1.00 | 198.32 | N |
| ATOM | 8095 | CA | SER | B | 2069 | −5.705 | −12.997 | 49.159 | 1.00 | 198.06 | C |
| ATOM | 8096 | C | SER | B | 2069 | −6.982 | −13.593 | 49.702 | 1.00 | 197.78 | C |
| ATOM | 8097 | O | SER | B | 2069 | −6.925 | −14.572 | 50.438 | 1.00 | 197.81 | O |
| ATOM | 8098 | CB | SER | B | 2069 | −5.699 | −13.133 | 47.644 | 1.00 | 198.11 | C |
| ATOM | 8099 | OG | SER | B | 2069 | −4.706 | −12.310 | 47.073 | 1.00 | 198.64 | O |
| ATOM | 8100 | N | TRP | B | 2070 | −8.121 | −12.980 | 49.344 | 1.00 | 197.52 | N |
| ATOM | 8101 | CA | TRP | B | 2070 | −9.477 | −13.529 | 49.580 | 1.00 | 197.24 | C |
| ATOM | 8102 | C | TRP | B | 2070 | −10.641 | −12.585 | 49.245 | 1.00 | 196.96 | C |
| ATOM | 8103 | O | TRP | B | 2070 | −10.517 | −11.363 | 49.383 | 1.00 | 196.87 | O |
| ATOM | 8104 | CB | TRP | B | 2070 | −9.644 | −13.999 | 51.023 | 1.00 | 197.49 | C |
| ATOM | 8105 | CG | TRP | B | 2070 | −9.427 | −12.923 | 52.020 | 1.00 | 197.62 | C |
| ATOM | 8106 | CD1 | TRP | B | 2070 | −8.263 | −12.628 | 52.652 | 1.00 | 197.82 | C |
| ATOM | 8107 | CD2 | TRP | B | 2070 | −10.399 | −11.991 | 52.507 | 1.00 | 197.76 | C |
| ATOM | 8108 | NE1 | TRP | B | 2070 | −8.445 | −11.573 | 53.505 | 1.00 | 198.12 | N |
| ATOM | 8109 | CE2 | TRP | B | 2070 | −9.749 | −11.162 | 53.436 | 1.00 | 197.96 | C |
| ATOM | 8110 | CE3 | TRP | B | 2070 | −11.758 | −11.778 | 52.248 | 1.00 | 198.14 | C |
| ATOM | 8111 | CZ2 | TRP | B | 2070 | −10.410 | −10.133 | 54.112 | 1.00 | 198.12 | C |
| ATOM | 8112 | CZ3 | TRP | B | 2070 | −12.413 | −10.756 | 52.916 | 1.00 | 198.00 | C |
| ATOM | 8113 | CH2 | TRP | B | 2070 | −11.740 | −9.949 | 53.840 | 1.00 | 198.02 | C |
| ATOM | 8114 | N | ILE | B | 2071 | −11.777 | −13.169 | 48.841 | 1.00 | 196.63 | N |
| ATOM | 8115 | CA | ILE | B | 2071 | −13.049 | −12.424 | 48.686 | 1.00 | 196.44 | C |
| ATOM | 8116 | C | ILE | B | 2071 | −14.229 | −12.969 | 49.510 | 1.00 | 196.19 | C |
| ATOM | 8117 | O | ILE | B | 2071 | −14.254 | −14.132 | 49.914 | 1.00 | 196.12 | O |
| ATOM | 8118 | CB | ILE | B | 2071 | −13.475 | −12.240 | 47.217 | 1.00 | 196.37 | C |
| ATOM | 8119 | CG1 | ILE | B | 2071 | −13.480 | −13.566 | 46.478 | 1.00 | 196.48 | C |
| ATOM | 8120 | CG2 | ILE | B | 2071 | −12.536 | −11.316 | 46.516 | 1.00 | 196.86 | C |
| ATOM | 8121 | CD1 | ILE | B | 2071 | −14.758 | −14.309 | 46.613 | 1.00 | 197.19 | C |
| ATOM | 8122 | N | LYS | B | 2072 | −15.215 | −12.107 | 49.727 | 1.00 | 195.92 | N |
| ATOM | 8123 | CA | LYS | B | 2072 | −16.192 | −12.295 | 50.790 | 1.00 | 195.59 | C |
| ATOM | 8124 | C | LYS | B | 2072 | −17.558 | −11.861 | 50.280 | 1.00 | 195.43 | C |
| ATOM | 8125 | O | LYS | B | 2072 | −17.716 | −10.766 | 49.725 | 1.00 | 195.67 | O |
| ATOM | 8126 | CB | LYS | B | 2072 | −15.771 | −11.462 | 52.022 | 1.00 | 195.78 | C |
| ATOM | 8127 | CG | LYS | B | 2072 | −16.442 | −11.795 | 53.359 | 1.00 | 195.51 | C |
| ATOM | 8128 | CD | LYS | B | 2072 | −15.576 | −11.393 | 54.575 | 1.00 | 195.20 | C |
| ATOM | 8129 | CE | LYS | B | 2072 | −15.861 | −9.987 | 55.092 | 1.00 | 194.29 | C |
| ATOM | 8130 | NZ | LYS | B | 2072 | −15.187 | −8.927 | 54.295 | 1.00 | 193.56 | N |
| ATOM | 8131 | N | VAL | B | 2073 | −18.541 | −12.731 | 50.460 | 1.00 | 194.92 | N |
| ATOM | 8132 | CA | VAL | B | 2073 | −19.905 | −12.448 | 50.042 | 1.00 | 194.46 | C |
| ATOM | 8133 | C | VAL | B | 2073 | −20.856 | −12.483 | 51.220 | 1.00 | 194.41 | C |
| ATOM | 8134 | O | VAL | B | 2073 | −21.097 | −13.561 | 51.796 | 1.00 | 194.68 | O |
| ATOM | 8135 | CB | VAL | B | 2073 | −20.418 | −13.488 | 49.028 | 1.00 | 194.26 | C |
| ATOM | 8136 | CG1 | VAL | B | 2073 | −20.592 | −12.870 | 47.659 | 1.00 | 194.34 | C |
| ATOM | 8137 | CG2 | VAL | B | 2073 | −19.507 | −14.695 | 48.994 | 1.00 | 193.67 | C |
| ATOM | 8138 | N | ASP | B | 2074 | −21.412 | −11.324 | 51.574 | 1.00 | 193.89 | N |
| ATOM | 8139 | CA | ASP | B | 2074 | −22.492 | −11.303 | 52.568 | 1.00 | 193.35 | C |
| ATOM | 8140 | C | ASP | B | 2074 | −23.808 | −11.757 | 51.931 | 1.00 | 192.84 | C |
| ATOM | 8141 | O | ASP | B | 2074 | −24.447 | −11.001 | 51.187 | 1.00 | 192.88 | O |
| ATOM | 8142 | CB | ASP | B | 2074 | −22.652 | −9.917 | 53.194 | 1.00 | 193.34 | C |
| ATOM | 8143 | CG | ASP | B | 2074 | −23.265 | −9.957 | 54.586 | 1.00 | 192.93 | C |
| ATOM | 8144 | OD1 | ASP | B | 2074 | −24.052 | −10.885 | 54.897 | 1.00 | 192.07 | O |
| ATOM | 8145 | OD2 | ASP | B | 2074 | −22.953 | −9.030 | 55.364 | 1.00 | 192.59 | O |
| ATOM | 8146 | N | LEU | B | 2075 | −24.197 | −12.996 | 52.213 | 1.00 | 191.95 | N |
| ATOM | 8147 | CA | LEU | B | 2075 | −25.463 | −13.511 | 51.736 | 1.00 | 191.23 | C |
| ATOM | 8148 | C | LEU | B | 2075 | −26.652 | −12.897 | 52.479 | 1.00 | 191.16 | C |
| ATOM | 8149 | O | LEU | B | 2075 | −27.799 | −13.271 | 52.226 | 1.00 | 191.33 | O |
| ATOM | 8150 | CB | LEU | B | 2075 | −25.489 | −15.031 | 51.832 | 1.00 | 190.96 | C |
| ATOM | 8151 | CG | LEU | B | 2075 | −25.488 | −15.773 | 50.506 | 1.00 | 190.34 | C |
| ATOM | 8152 | CD1 | LEU | B | 2075 | −25.198 | −17.240 | 50.727 | 1.00 | 189.81 | C |
| ATOM | 8153 | CD2 | LEU | B | 2075 | −26.823 | −15.591 | 49.798 | 1.00 | 190.14 | C |
| ATOM | 8154 | N | LEU | B | 2076 | −26.366 | −11.960 | 53.389 | 1.00 | 190.77 | N |
| ATOM | 8155 | CA | LEU | B | 2076 | −27.381 | −11.200 | 54.142 | 1.00 | 190.47 | C |
| ATOM | 8156 | C | LEU | B | 2076 | −28.425 | −12.045 | 54.876 | 1.00 | 190.39 | C |
| ATOM | 8157 | O | LEU | B | 2076 | −29.517 | −11.552 | 55.168 | 1.00 | 190.50 | O |
| ATOM | 8158 | CB | LEU | B | 2076 | −28.109 | −10.184 | 53.246 | 1.00 | 190.33 | C |
| ATOM | 8159 | CG | LEU | B | 2076 | −27.387 | −9.129 | 52.402 | 1.00 | 190.51 | C |
| ATOM | 8160 | CD1 | LEU | B | 2076 | −26.189 | −8.531 | 53.140 | 1.00 | 190.74 | C |
| ATOM | 8161 | CD2 | LEU | B | 2076 | −26.989 | −9.667 | 51.013 | 1.00 | 190.36 | C |
| ATOM | 8162 | N | ALA | B | 2077 | −28.090 | −13.302 | 55.170 | 1.00 | 190.17 | N |
| ATOM | 8163 | CA | ALA | B | 2077 | −28.985 | −14.237 | 55.860 | 1.00 | 189.97 | C |
| ATOM | 8164 | C | ALA | B | 2077 | −28.340 | −15.621 | 55.915 | 1.00 | 189.90 | C |
| ATOM | 8165 | O | ALA | B | 2077 | −27.655 | −16.005 | 54.972 | 1.00 | 189.89 | O |
| ATOM | 8166 | CB | ALA | B | 2077 | −30.337 | −14.317 | 55.146 | 1.00 | 189.90 | C |
| ATOM | 8167 | N | PRO | B | 2078 | −28.536 | −16.369 | 57.024 | 1.00 | 189.85 | N |
| ATOM | 8168 | CA | PRO | B | 2078 | −28.105 | −17.771 | 57.037 | 1.00 | 189.67 | C |
| ATOM | 8169 | C | PRO | B | 2078 | −28.733 | −18.573 | 55.885 | 1.00 | 189.65 | C |
| ATOM | 8170 | O | PRO | B | 2078 | −29.953 | −18.669 | 55.792 | 1.00 | 189.54 | O |
| ATOM | 8171 | CB | PRO | B | 2078 | −28.584 | −18.283 | 58.404 | 1.00 | 189.57 | C |
| ATOM | 8172 | CG | PRO | B | 2078 | −29.545 | −17.257 | 58.920 | 1.00 | 189.81 | C |
| ATOM | 8173 | CD | PRO | B | 2078 | −29.121 | −15.959 | 58.317 | 1.00 | 190.00 | C |
| ATOM | 8174 | N | MET | B | 2079 | −27.882 | −19.134 | 55.025 | 1.00 | 189.82 | N |
| ATOM | 8175 | CA | MET | B | 2079 | −28.279 | −19.772 | 53.764 | 1.00 | 189.89 | C |
| ATOM | 8176 | C | MET | B | 2079 | −27.636 | −21.140 | 53.595 | 1.00 | 189.95 | C |
| ATOM | 8177 | O | MET | B | 2079 | −26.559 | −21.381 | 54.140 | 1.00 | 189.75 | O |
| ATOM | 8178 | CB | MET | B | 2079 | −27.801 | −18.906 | 52.609 | 1.00 | 190.06 | C |
| ATOM | 8179 | CG | MET | B | 2079 | −28.675 | −17.740 | 52.290 | 1.00 | 190.33 | C |
| ATOM | 8180 | SD | MET | B | 2079 | −30.006 | −18.318 | 51.249 | 1.00 | 192.21 | S |
| ATOM | 8181 | CE | MET | B | 2079 | −30.382 | −16.843 | 50.285 | 1.00 | 191.88 | C |
| ATOM | 8182 | N | ILE | B | 2080 | −28.275 | −22.026 | 52.825 | 1.00 | 190.22 | N |
| ATOM | 8183 | CA | ILE | B | 2080 | −27.643 | −23.302 | 52.441 | 1.00 | 190.73 | C |
| ATOM | 8184 | C | ILE | B | 2080 | −26.856 | −23.128 | 51.151 | 1.00 | 190.98 | C |
| ATOM | 8185 | O | ILE | B | 2080 | −27.423 | −22.737 | 50.129 | 1.00 | 191.18 | O |
| ATOM | 8186 | CB | ILE | B | 2080 | −28.647 | −24.474 | 52.189 | 1.00 | 190.78 | C |
| ATOM | 8187 | CG1 | ILE | B | 2080 | −29.691 | −24.603 | 53.301 | 1.00 | 191.07 | C |
| ATOM | 8188 | CG2 | ILE | B | 2080 | −27.883 | −25.805 | 51.985 | 1.00 | 190.82 | C |
| ATOM | 8189 | CD1 | ILE | B | 2080 | −30.599 | −25.826 | 53.154 | 1.00 | 190.75 | C |
| ATOM | 8190 | N | ILE | B | 2081 | −25.562 | −23.436 | 51.183 | 1.00 | 191.20 | N |
| ATOM | 8191 | CA | ILE | B | 2081 | −24.737 | −23.325 | 49.979 | 1.00 | 191.47 | C |
| ATOM | 8192 | C | ILE | B | 2081 | −24.213 | −24.705 | 49.528 | 1.00 | 191.59 | C |
| ATOM | 8193 | O | ILE | B | 2081 | −23.326 | −25.281 | 50.171 | 1.00 | 191.74 | O |
| ATOM | 8194 | CB | ILE | B | 2081 | −23.617 | −22.222 | 50.108 | 1.00 | 191.46 | C |
| ATOM | 8195 | CG1 | ILE | B | 2081 | −22.567 | −22.588 | 51.157 | 1.00 | 191.66 | C |
| ATOM | 8196 | CG2 | ILE | B | 2081 | −24.220 | −20.844 | 50.448 | 1.00 | 191.10 | C |
| ATOM | 8197 | CD1 | ILE | B | 2081 | −21.318 | −21.710 | 51.119 | 1.00 | 191.77 | C |
| ATOM | 8198 | N | HIS | B | 2082 | −24.800 | −25.226 | 48.439 | 1.00 | 191.62 | N |
| ATOM | 8199 | CA | HIS | B | 2082 | −24.525 | −26.589 | 47.909 | 1.00 | 191.35 | C |
| ATOM | 8200 | C | HIS | B | 2082 | −23.285 | −26.691 | 47.044 | 1.00 | 191.13 | C |
| ATOM | 8201 | O | HIS | B | 2082 | −22.782 | −27.802 | 46.822 | 1.00 | 190.98 | O |
| ATOM | 8202 | CB | HIS | B | 2082 | −25.676 | −27.080 | 47.039 | 1.00 | 191.32 | C |
| ATOM | 8203 | CG | HIS | B | 2082 | −26.819 | −27.648 | 47.805 | 1.00 | 191.25 | C |
| ATOM | 8204 | ND1 | HIS | B | 2082 | −27.807 | −26.861 | 48.355 | 1.00 | 191.05 | N |
| ATOM | 8205 | CD2 | HIS | B | 2082 | −27.152 | −28.927 | 48.085 | 1.00 | 191.74 | C |
| ATOM | 8206 | CE1 | HIS | B | 2082 | −28.695 | −27.632 | 48.954 | 1.00 | 191.41 | C |
| ATOM | 8207 | NE2 | HIS | B | 2082 | −28.321 | −28.890 | 48.805 | 1.00 | 192.02 | N |
| ATOM | 8208 | N | GLY | B | 2083 | −22.839 | −25.538 | 46.529 | 1.00 | 190.82 | N |
| ATOM | 8209 | CA | GLY | B | 2083 | −21.717 | −25.454 | 45.594 | 1.00 | 190.37 | C |
| ATOM | 8210 | C | GLY | B | 2083 | −21.411 | −24.065 | 45.054 | 1.00 | 190.04 | C |
| ATOM | 8211 | O | GLY | B | 2083 | −22.092 | −23.085 | 45.383 | 1.00 | 189.95 | O |
| ATOM | 8212 | N | ILE | B | 2084 | −20.378 | −23.994 | 44.211 | 1.00 | 189.73 | N |
| ATOM | 8213 | CA | ILE | B | 2084 | −19.853 | −22.729 | 43.674 | 1.00 | 189.35 | C |
| ATOM | 8214 | C | ILE | B | 2084 | −19.250 | −22.926 | 42.278 | 1.00 | 189.36 | C |
| ATOM | 8215 | O | ILE | B | 2084 | −18.235 | −23.620 | 42.113 | 1.00 | 189.58 | O |
| ATOM | 8216 | CB | ILE | B | 2084 | −18.791 | −22.106 | 44.627 | 1.00 | 189.22 | C |
| ATOM | 8217 | CG1 | ILE | B | 2084 | −18.287 | −20.768 | 44.109 | 1.00 | 188.26 | C |
| ATOM | 8218 | CG2 | ILE | B | 2084 | −17.610 | −23.057 | 44.854 | 1.00 | 189.35 | C |
| ATOM | 8219 | CD1 | ILE | B | 2084 | −17.344 | −20.103 | 45.074 | 1.00 | 186.93 | C |
| ATOM | 8220 | N | LYS | B | 2085 | −19.897 | −22.335 | 41.276 | 1.00 | 189.04 | N |
| ATOM | 8221 | CA | LYS | B | 2085 | −19.355 | −22.311 | 39.927 | 1.00 | 188.42 | C |
| ATOM | 8222 | C | LYS | B | 2085 | −18.297 | −21.224 | 39.907 | 1.00 | 188.44 | C |
| ATOM | 8223 | O | LYS | B | 2085 | −18.528 | −20.061 | 40.267 | 1.00 | 187.96 | O |
| ATOM | 8224 | CB | LYS | B | 2085 | −20.441 | −22.061 | 38.884 | 1.00 | 188.19 | C |
| ATOM | 8225 | CG | LYS | B | 2085 | −21.402 | −23.201 | 38.711 | 1.00 | 187.37 | C |
| ATOM | 8226 | CD | LYS | B | 2085 | −22.819 | −22.690 | 38.706 | 1.00 | 187.60 | C |
| ATOM | 8227 | CE | LYS | B | 2085 | −23.820 | −23.774 | 38.323 | 1.00 | 188.94 | C |
| ATOM | 8228 | NZ | LYS | B | 2085 | −24.442 | −23.600 | 36.964 | 1.00 | 190.31 | N |
| ATOM | 8229 | N | THR | B | 2086 | −17.112 | −21.647 | 39.518 | 1.00 | 188.64 | N |
| ATOM | 8230 | CA | THR | B | 2086 | −15.954 | −20.794 | 39.530 | 1.00 | 188.99 | C |
| ATOM | 8231 | C | THR | B | 2086 | −15.406 | −20.748 | 38.106 | 1.00 | 188.99 | C |
| ATOM | 8232 | O | THR | B | 2086 | −15.478 | −21.737 | 37.375 | 1.00 | 188.86 | O |
| ATOM | 8233 | CB | THR | B | 2086 | −14.909 | −21.236 | 40.647 | 1.00 | 189.17 | C |
| ATOM | 8234 | OG1 | THR | B | 2086 | −13.570 | −21.180 | 40.145 | 1.00 | 189.75 | O |
| ATOM | 8235 | CG2 | THR | B | 2O86 | −15.174 | −22.658 | 41.194 | 1.00 | 189.24 | C |
| ATOM | 8236 | N | GLN | B | 2087 | −14.903 | −19.582 | 37.712 | 1.00 | 189.20 | N |
| ATOM | 8237 | CA | GLN | B | 2087 | −14.408 | −19.350 | 36.363 | 1.00 | 189.70 | C |
| ATOM | 8238 | C | GLN | B | 2087 | −13.278 | −18.317 | 36.377 | 1.00 | 190.00 | C |
| ATOM | 8239 | O | GLN | B | 2087 | −13.053 | −17.661 | 37.396 | 1.00 | 190.15 | O |
| ATOM | 8240 | CB | GLN | B | 2087 | −15.552 | −18.864 | 35.491 | 1.00 | 189.72 | C |
| ATOM | 8241 | CG | GLN | B | 2087 | −15.283 | −18.914 | 34.019 | 1.00 | 190.36 | C |
| ATOM | 8242 | CD | GLN | B | 2087 | −16.340 | −18.185 | 33.240 | 1.00 | 191.47 | C |
| ATOM | 8243 | OE1 | GLN | B | 2087 | −16.390 | −16.953 | 33.231 | 1.00 | 191.27 | O |
| ATOM | 8244 | NE2 | GLN | B | 2087 | −17.204 | −18.943 | 32.575 | 1.00 | 192.32 | N |
| ATOM | 8245 | N | GLY | B | 2088 | −12.570 | −18.174 | 35.252 | 1.00 | 190.39 | N |
| ATOM | 8246 | CA | GLY | B | 2088 | −11.434 | −17.234 | 35.143 | 1.00 | 190.72 | C |
| ATOM | 8247 | C | GLY | B | 2088 | −11.570 | −16.144 | 34.087 | 1.00 | 190.80 | C |
| ATOM | 8248 | O | GLY | B | 2088 | −12.496 | −16.158 | 33.271 | 1.00 | 190.82 | O |
| ATOM | 8249 | N | ALA | B | 2089 | −10.644 | −15.193 | 34.096 | 1.00 | 190.83 | N |
| ATOM | 8250 | CA | ALA | B | 2089 | −10.709 | −14.105 | 33.132 | 1.00 | 191.22 | C |
| ATOM | 8251 | C | ALA | B | 2089 | −9.639 | −14.230 | 32.032 | 1.00 | 191.55 | C |
| ATOM | 8252 | O | ALA | B | 2089 | −8.882 | −15.211 | 32.020 | 1.00 | 191.77 | O |
| ATOM | 8253 | CB | ALA | B | 2089 | −10.621 | −12.779 | 33.840 | 1.00 | 191.25 | C |
| ATOM | 8254 | N | ARG | B | 2090 | −9.591 | −13.239 | 31.124 | 1.00 | 191.65 | N |
| ATOM | 8255 | CA | ARG | B | 2090 | −8.697 | −13.222 | 29.938 | 1.00 | 191.52 | C |
| ATOM | 8256 | C | ARG | B | 2090 | −8.086 | −11.829 | 29.716 | 1.00 | 191.27 | C |
| ATOM | 8257 | O | ARG | B | 2090 | −8.824 | −10.846 | 29.604 | 1.00 | 191.25 | O |
| ATOM | 8258 | CB | ARG | B | 2090 | −9.480 | −13.669 | 28.692 | 1.00 | 191.64 | C |
| ATOM | 8259 | CG | ARG | B | 2090 | −8.651 | −13.953 | 27.449 | 1.00 | 192.08 | C |
| ATOM | 8260 | CD | ARG | B | 2090 | −8.865 | −12.885 | 26.392 | 1.00 | 193.59 | C |
| ATOM | 8261 | NE | ARG | B | 2090 | −7.799 | −12.863 | 25.386 | 1.00 | 195.54 | N |
| ATOM | 8262 | CZ | ARG | B | 2090 | −7.908 | −13.320 | 24.137 | 1.00 | 196.42 | C |
| ATOM | 8263 | NH1 | ARG | B | 2090 | −9.050 | −13.851 | 23.711 | 1.00 | 197.24 | N |
| ATOM | 8264 | NH2 | ARG | B | 2090 | −6.871 | −13.244 | 23.304 | 1.00 | 196.28 | N |
| ATOM | 8265 | N | GLN | B | 2091 | −6.752 | −11.754 | 29.642 | 1.00 | 190.99 | N |
| ATOM | 8266 | CA | GLN | B | 2091 | −6.027 | −10.465 | 29.593 | 1.00 | 190.79 | C |
| ATOM | 8267 | C | GLN | B | 2091 | −5.594 | −10.027 | 28.210 | 1.00 | 190.72 | C |
| ATOM | 8268 | O | GLN | B | 2091 | −6.292 | −10.261 | 27.230 | 1.00 | 190.71 | O |
| ATOM | 8269 | CB | GLN | B | 2091 | −4.795 | −10.484 | 30.505 | 1.00 | 190.83 | C |
| ATOM | 8270 | CG | GLN | B | 2091 | −4.869 | −9.561 | 31.740 | 1.00 | 190.67 | C |
| ATOM | 8271 | CD | GLN | B | 2091 | −4.374 | −8.138 | 31.484 | 1.00 | 189.97 | C |
| ATOM | 8272 | OE1 | GLN | B | 2091 | −4.992 | −7.376 | 30.734 | 1.00 | 189.40 | O |
| ATOM | 8273 | NE2 | GLN | B | 2091 | −3.264 | −7.768 | 32.134 | 1.00 | 189.72 | N |
| ATOM | 8274 | N | LYS | B | 2092 | −4.436 | −9.370 | 28.159 | 1.00 | 190.79 | N |
| ATOM | 8275 | CA | LYS | B | 2092 | −3.847 | −8.855 | 26.923 | 1.00 | 190.97 | C |
| ATOM | 8276 | C | LYS | B | 2092 | −3.809 | −9.988 | 25.886 | 1.00 | 191.15 | C |
| ATOM | 8277 | O | LYS | B | 2092 | −4.524 | −9.945 | 24.878 | 1.00 | 191.25 | O |
| ATOM | 8278 | CB | LYS | B | 2092 | −2.457 | −8.232 | 27.216 | 1.00 | 190.84 | C |
| ATOM | 8279 | CG | LYS | B | 2092 | −1.647 | −7.688 | 26.027 | 1.00 | 190.30 | C |
| ATOM | 8280 | CD | LYS | B | 2092 | −2.307 | −6.515 | 25.304 | 1.00 | 189.68 | C |
| ATOM | 8281 | CE | LYS | B | 2092 | −2.890 | −6.954 | 23.964 | 1.00 | 189.09 | C |
| ATOM | 8282 | NZ | LYS | B | 2092 | −3.102 | −5.830 | 23.014 | 1.00 | 188.71 | N |
| ATOM | 8283 | N | PHE | B | 2093 | −2.997 | −11.005 | 26.164 | 1.00 | 191.22 | N |
| ATOM | 8284 | CA | PHE | B | 2093 | −3.006 | −12.266 | 25.425 | 1.00 | 191.17 | C |
| ATOM | 8285 | C | PHE | B | 2093 | −2.742 | −13.368 | 26.443 | 1.00 | 191.05 | C |
| ATOM | 8286 | O | PHE | B | 2093 | −2.315 | −14.472 | 26.101 | 1.00 | 190.90 | O |
| ATOM | 8287 | CB | PHE | B | 2093 | −1.955 | −12.267 | 24.298 | 1.00 | 191.34 | C |
| ATOM | 8288 | CG | PHE | B | 2093 | −2.406 | −11.556 | 23.039 | 1.00 | 191.51 | C |
| ATOM | 8289 | CD1 | PHE | B | 2093 | −1.927 | −10.281 | 22.733 | 1.00 | 191.52 | C |
| ATOM | 8290 | CD2 | PHE | B | 2093 | −3.315 | −12.161 | 22.164 | 1.00 | 191.52 | C |
| ATOM | 8291 | CE1 | PHE | B | 2093 | −2.348 | −9.618 | 21.578 | 1.00 | 191.44 | C |
| ATOM | 8292 | CE2 | PHE | B | 2093 | −3.743 | −11.509 | 21.011 | 1.00 | 191.37 | C |
| ATOM | 8293 | CZ | PHE | B | 2093 | −3.257 | −10.233 | 20.717 | 1.00 | 191.49 | C |
| ATOM | 8294 | N | SER | B | 2094 | −3.023 | −13.043 | 27.703 | 1.00 | 191.04 | N |
| ATOM | 8295 | CA | SER | B | 2094 | −2.672 | −13.885 | 28.837 | 1.00 | 191.02 | C |
| ATOM | 8296 | C | SER | B | 2094 | −3.868 | −14.673 | 29.374 | 1.00 | 191.12 | C |
| ATOM | 8297 | O | SER | B | 2094 | −4.955 | −14.118 | 29.575 | 1.00 | 191.11 | O |
| ATOM | 8298 | CB | SER | B | 2094 | −2.082 | −13.023 | 29.952 | 1.00 | 190.91 | C |
| ATOM | 8299 | OG | SER | B | 2094 | −1.103 | −12.129 | 29.464 | 1.00 | 190.50 | O |
| ATOM | 8300 | N | SER | B | 2095 | −3.658 | −15.966 | 29.601 | 1.00 | 191.13 | N |
| ATOM | 8301 | CA | SER | B | 2095 | −4.662 | −16.813 | 30.225 | 1.00 | 191.28 | C |
| ATOM | 8302 | C | SER | B | 2095 | −4.422 | −16.729 | 31.731 | 1.00 | 191.42 | C |
| ATOM | 8303 | O | SER | B | 2095 | −3.388 | −17.207 | 32.198 | 1.00 | 191.69 | O |
| ATOM | 8304 | CB | SER | B | 2095 | −4.501 | −18.258 | 29.730 | 1.00 | 191.27 | C |
| ATOM | 8305 | OG | SER | B | 2095 | −5.717 | −18.990 | 29.785 | 1.00 | 191.27 | O |
| ATOM | 8306 | N | LEU | B | 2096 | −5.338 | −16.107 | 32.487 | 1.00 | 191.42 | N |
| ATOM | 8307 | CA | LEU | B | 2096 | −5.189 | −16.022 | 33.960 | 1.00 | 191.32 | C |
| ATOM | 8308 | C | LEU | B | 2096 | −6.445 | −16.300 | 34.800 | 1.00 | 191.69 | C |
| ATOM | 8309 | O | LEU | B | 2096 | −7.538 | −15.775 | 34.551 | 1.00 | 191.66 | O |
| ATOM | 8310 | CB | LEU | B | 2096 | −4.504 | −14.723 | 34.399 | 1.00 | 190.87 | C |
| ATOM | 8311 | CG | LEU | B | 2096 | −5.321 | −13.451 | 34.558 | 1.00 | 190.02 | C |
| ATOM | 8312 | CD1 | LEU | B | 2096 | −4.441 | −12.402 | 35.158 | 1.00 | 189.81 | C |
| ATOM | 8313 | CD2 | LEU | B | 2096 | −5.880 | −12.977 | 33.240 | 1.00 | 189.03 | C |
| ATOM | 8314 | N | TYR | B | 2097 | −6.237 | −17.132 | 35.818 | 1.00 | 192.15 | N |
| ATOM | 8315 | CA | TYR | B | 2097 | −7.301 | −17.775 | 36.597 | 1.00 | 192.58 | C |
| ATOM | 8316 | C | TYR | B | 2097 | −6.729 | −18.210 | 37.970 | 1.00 | 192.76 | C |
| ATOM | 8317 | O | TYR | B | 2097 | −5.545 | −17.969 | 38.244 | 1.00 | 193.00 | O |
| ATOM | 8318 | CB | TYR | B | 2097 | −7.880 | −18.976 | 35.808 | 1.00 | 192.64 | C |
| ATOM | 8319 | CG | TYR | B | 2097 | −6.835 | −19.928 | 35.222 | 1.00 | 192.51 | C |
| ATOM | 8320 | CD1 | TYR | B | 2097 | −6.486 | −21.105 | 35.883 | 1.00 | 192.37 | C |
| ATOM | 8321 | CD2 | TYR | B | 2097 | −6.203 | −19.650 | 34.012 | 1.00 | 192.30 | C |
| ATOM | 8322 | CE1 | TYR | B | 2097 | −5.530 | −21.973 | 35.361 | 1.00 | 192.25 | C |
| ATOM | 8323 | CE2 | TYR | B | 2097 | −5.240 | −20.511 | 33.487 | 1.00 | 192.52 | C |
| ATOM | 8324 | CZ | TYR | B | 2097 | −4.912 | −21.669 | 34.168 | 1.00 | 192.53 | C |
| ATOM | 8325 | OH | TYR | B | 2097 | −3.966 | −22.527 | 33.652 | 1.00 | 192.96 | O |
| ATOM | 8326 | N | ILE | B | 2098 | −7.555 | −18.810 | 38.835 | 1.00 | 192.64 | N |
| ATOM | 8327 | CA | ILE | B | 2098 | −7.062 | −19.423 | 40.083 | 1.00 | 192.42 | C |
| ATOM | 8328 | C | ILE | B | 2098 | −7.171 | −20.920 | 39.950 | 1.00 | 192.39 | C |
| ATOM | 8329 | O | ILE | B | 2098 | −8.192 | −21.436 | 39.494 | 1.00 | 192.10 | O |
| ATOM | 8330 | CB | ILE | B | 2098 | −7.816 | −18.955 | 41.343 | 1.00 | 192.37 | C |
| ATOM | 8331 | CG1 | ILE | B | 2098 | −9.178 | −18.366 | 40.976 | 1.00 | 192.84 | C |
| ATOM | 8332 | CG2 | ILE | B | 2098 | −7.024 | −17.897 | 42.078 | 1.00 | 192.01 | C |
| ATOM | 8333 | CD1 | ILE | B | 2098 | −10.244 | −19.390 | 40.640 | 1.00 | 193.31 | C |
| ATOM | 8334 | N | SER | B | 2099 | −6.108 | −21.614 | 40.333 | 1.00 | 192.67 | N |
| ATOM | 8335 | CA | SER | B | 2099 | −5.986 | −23.048 | 40.051 | 1.00 | 193.13 | C |
| ATOM | 8336 | C | SER | B | 2099 | −6.392 | −23.924 | 41.228 | 1.00 | 193.28 | C |
| ATOM | 8337 | O | SER | B | 2099 | −6.741 | −25.106 | 41.060 | 1.00 | 193.35 | O |
| ATOM | 8338 | CB | SER | B | 2099 | −4.557 | −23.394 | 39.610 | 1.00 | 193.16 | C |
| ATOM | 8339 | OG | SER | B | 2099 | −3.601 | −22.910 | 40.530 | 1.00 | 192.95 | O |
| ATOM | 8340 | N | GLN | B | 2100 | −6.334 | −23.332 | 42.415 | 1.00 | 193.29 | N |
| ATOM | 8341 | CA | GLN | B | 2100 | −6.612 | −24.040 | 43.646 | 1.00 | 193.21 | C |
| ATOM | 8342 | C | GLN | B | 2100 | −7.131 | −23.096 | 44.708 | 1.00 | 192.89 | C |
| ATOM | 8343 | O | GLN | B | 2100 | −6.712 | −21.937 | 44.775 | 1.00 | 192.79 | O |
| ATOM | 8344 | CB | GLN | B | 2100 | −5.352 | −24.716 | 44.147 | 1.00 | 193.35 | C |
| ATOM | 8345 | CG | GLN | B | 2100 | −5.139 | −26.077 | 43.576 | 1.00 | 194.48 | C |
| ATOM | 8346 | CD | GLN | B | 2100 | −4.670 | −27.044 | 44.627 | 1.00 | 196.82 | C |
| ATOM | 8347 | OE1 | GLN | B | 2100 | −4.068 | −26.651 | 45.637 | 1.00 | 197.94 | O |
| ATOM | 8348 | NE2 | GLN | B | 2100 | −4.949 | −28.324 | 44.410 | 1.00 | 197.99 | N |
| ATOM | 8349 | N | PHE | B | 2101 | −8.048 | −23.597 | 45.535 | 1.00 | 192.60 | N |
| ATOM | 8350 | CA | PHE | B | 2101 | −8.644 | −22.786 | 46.599 | 1.00 | 192.27 | C |
| ATOM | 8351 | C | PHE | B | 2101 | −9.256 | −23.560 | 47.761 | 1.00 | 192.12 | C |
| ATOM | 8352 | O | PHE | B | 2101 | −9.758 | −24.673 | 47.571 | 1.00 | 192.29 | O |
| ATOM | 8353 | CB | PHE | B | 2101 | −9.646 | −21.753 | 46.038 | 1.00 | 192.21 | C |
| ATOM | 8354 | CG | PHE | B | 2101 | −10.886 | −22.338 | 45.403 | 1.00 | 191.55 | C |
| ATOM | 8355 | CD1 | PHE | B | 2101 | −12.126 | −22.089 | 45.950 | 1.00 | 190.68 | C |
| ATOM | 8356 | CD2 | PHE | B | 2101 | −10.819 | −23.078 | 44.229 | 1.00 | 191.88 | C |
| ATOM | 8357 | CE1 | PHE | B | 2101 | −13.273 | −22.581 | 45.355 | 1.00 | 191.29 | C |
| ATOM | 8358 | CE2 | PHE | B | 2101 | −11.971 | −23.582 | 43.628 | 1.00 | 191.93 | C |
| ATOM | 8359 | CZ | PHE | B | 2101 | −13.196 | −23.332 | 44.190 | 1.00 | 191.74 | C |
| ATOM | 8360 | N | ILE | B | 2102 | −9.183 | −22.971 | 48.961 | 1.00 | 191.57 | N |
| ATOM | 8361 | CA | ILE | B | 2102 | −9.924 | −23.461 | 50.121 | 1.00 | 190.96 | C |
| ATOM | 8362 | C | ILE | B | 2102 | −11.062 | −22.525 | 50.482 | 1.00 | 191.10 | C |
| ATOM | 8363 | O | ILE | B | 2102 | −11.028 | −21.325 | 50.193 | 1.00 | 190.87 | O |
| ATOM | 8364 | CB | ILE | B | 2102 | −9.051 | −23.659 | 51.341 | 1.00 | 190.57 | C |
| ATOM | 8365 | CG1 | ILE | B | 2102 | −8.269 | −22.388 | 51.631 | 1.00 | 190.15 | C |
| ATOM | 8366 | CG2 | ILE | B | 2102 | −8.144 | −24.846 | 51.137 | 1.00 | 190.42 | C |
| ATOM | 8367 | CD1 | ILE | B | 2102 | −7.992 | −22.181 | 53.090 | 1.00 | 190.79 | C |
| ATOM | 8368 | N | ILE | B | 2103 | −12.074 | −23.093 | 51.122 | 1.00 | 191.36 | N |
| ATOM | 8369 | CA | ILE | B | 2103 | −13.285 | −22.362 | 51.398 | 1.00 | 191.79 | C |
| ATOM | 8370 | C | ILE | B | 2103 | −13.611 | −22.329 | 52.878 | 1.00 | 192.20 | C |
| ATOM | 8371 | O | ILE | B | 2103 | −13.891 | −23.367 | 53.471 | 1.00 | 192.32 | O |
| ATOM | 8372 | CB | ILE | B | 2103 | −14.434 | −22.946 | 50.606 | 1.00 | 191.68 | C |
| ATOM | 8373 | CG1 | ILE | B | 2103 | −14.361 | −22.387 | 49.193 | 1.00 | 192.33 | C |
| ATOM | 8374 | CG2 | ILE | B | 2103 | −15.772 | −22.605 | 51.242 | 1.00 | 191.76 | C |
| ATOM | 8375 | CD1 | ILE | B | 2103 | −15.500 | −22.806 | 48.304 | 1.00 | 193.80 | C |
| ATOM | 8376 | N | MET | B | 2104 | −13.552 | −21.125 | 53.454 | 1.00 | 192.63 | N |
| ATOM | 8377 | CA | MET | B | 2104 | −13.958 | −20.855 | 54.835 | 1.00 | 192.85 | C |
| ATOM | 8378 | C | MET | B | 2104 | −15.318 | −20.168 | 54.861 | 1.00 | 193.29 | C |
| ATOM | 8379 | O | MET | B | 2104 | −15.698 | −19.489 | 53.899 | 1.00 | 193.19 | O |
| ATOM | 8380 | CB | MET | B | 2104 | −12.963 | −19.930 | 55.509 | 1.00 | 192.69 | C |
| ATOM | 8381 | CG | MET | B | 2104 | −11.676 | −20.550 | 55.904 | 1.00 | 192.26 | C |
| ATOM | 8382 | SD | MET | B | 2104 | −10.627 | −19.148 | 56.224 | 1.00 | 192.87 | S |
| ATOM | 8383 | CE | MET | B | 2104 | −9.173 | −19.944 | 56.905 | 1.00 | 193.30 | C |
| ATOM | 8384 | N | TYR | B | 2105 | −16.039 | −20.327 | 55.971 | 1.00 | 193.90 | N |
| ATOM | 8385 | CA | TYR | B | 2105 | −17.335 | −19.674 | 56.132 | 1.00 | 194.39 | C |
| ATOM | 8386 | C | TYR | B | 2105 | −17.654 | −19.280 | 57.565 | 1.00 | 194.62 | C |
| ATOM | 8387 | O | TYR | B | 2105 | −17.175 | −19.890 | 58.533 | 1.00 | 194.25 | O |
| ATOM | 8388 | CB | TYR | B | 2105 | −18.461 | −20.537 | 55.558 | 1.00 | 194.57 | C |
| ATOM | 8389 | CG | TYR | B | 2105 | −18.567 | −21.885 | 56.207 | 1.00 | 194.87 | C |
| ATOM | 8390 | CD1 | TYR | B | 2105 | −19.350 | −22.064 | 57.338 | 1.00 | 195.45 | C |
| ATOM | 8391 | CD2 | TYR | B | 2105 | −17.878 | −22.985 | 55.695 | 1.00 | 194.73 | C |
| ATOM | 8392 | CE1 | TYR | B | 2105 | −19.452 | −23.296 | 57.945 | 1.00 | 195.58 | C |
| ATOM | 8393 | CE2 | TYR | B | 2105 | −17.970 | −24.223 | 56.294 | 1.00 | 194.78 | C |
| ATOM | 8394 | CZ | TYR | B | 2105 | −18.761 | −24.366 | 57.418 | 1.00 | 195.29 | C |
| ATOM | 8395 | OH | TYR | B | 2105 | −18.872 | −25.577 | 58.038 | 1.00 | 195.61 | O |
| ATOM | 8396 | N | SER | B | 2106 | −18.462 | −18.232 | 57.661 | 1.00 | 195.20 | N |
| ATOM | 8397 | CA | SER | B | 2106 | −19.011 | −17.763 | 58.918 | 1.00 | 196.00 | C |
| ATOM | 8398 | C | SER | B | 2106 | −20.527 | −17.681 | 58.840 | 1.00 | 196.29 | C |
| ATOM | 8399 | O | SER | B | 2106 | −21.091 | −17.247 | 57.826 | 1.00 | 196.31 | O |
| ATOM | 8400 | CB | SER | B | 2106 | −18.435 | −16.398 | 59.305 | 1.00 | 196.11 | C |
| ATOM | 8401 | OG | SER | B | 2106 | −17.471 | −16.529 | 60.338 | 1.00 | 196.58 | O |
| ATOM | 8402 | N | LEU | B | 2107 | −21.167 | −18.117 | 59.924 | 1.00 | 196.63 | N |
| ATOM | 8403 | CA | LEU | B | 2107 | −22.614 | −18.077 | 60.065 | 1.00 | 196.85 | C |
| ATOM | 8404 | C | LEU | B | 2107 | −23.009 | −16.815 | 60.799 | 1.00 | 197.28 | C |
| ATOM | 8405 | O | LEU | B | 2107 | −24.196 | −16.544 | 60.967 | 1.00 | 197.50 | O |
| ATOM | 8406 | CB | LEU | B | 2107 | −23.133 | −19.317 | 60.815 | 1.00 | 196.62 | C |
| ATOM | 8407 | CG | LEU | B | 2107 | −22.207 | −20.190 | 61.680 | 1.00 | 196.44 | C |
| ATOM | 8408 | CD1 | LEU | B | 2107 | −21.770 | −19.521 | 62.972 | 1.00 | 195.90 | C |
| ATOM | 8409 | CD2 | LEU | B | 2107 | −22.881 | −21.525 | 61.986 | 1.00 | 196.80 | C |
| ATOM | 8410 | N | ASP | B | 2108 | −22.007 | −16.023 | 61.186 | 1.00 | 197.73 | N |
| ATOM | 8411 | CA | ASP | B | 2108 | −22.174 | −14.996 | 62.215 | 1.00 | 198.22 | C |
| ATOM | 8412 | C | ASP | B | 2108 | −21.365 | −13.725 | 62.005 | 1.00 | 198.24 | C |
| ATOM | 8413 | O | ASP | B | 2108 | −21.811 | −12.640 | 62.382 | 1.00 | 198.09 | O |
| ATOM | 8414 | CB | ASP | B | 2108 | −21.791 | −15.587 | 63.572 | 1.00 | 198.51 | C |
| ATOM | 8415 | CG | ASP | B | 2108 | −20.490 | −16.394 | 63.519 | 1.00 | 199.27 | C |
| ATOM | 8416 | OD1 | ASP | B | 2108 | −19.821 | −16.476 | 64.572 | 1.00 | 200.89 | O |
| ATOM | 8417 | OD2 | ASP | B | 2108 | −20.141 | −16.947 | 62.444 | 1.00 | 198.94 | O |
| ATOM | 8418 | N | GLY | B | 2109 | −20.169 | −13.875 | 61.440 | 1.00 | 198.44 | N |
| ATOM | 8419 | CA | GLY | B | 2109 | −19.252 | −12.754 | 61.244 | 1.00 | 198.84 | C |
| ATOM | 8420 | C | GLY | B | 2109 | −18.033 | −12.782 | 62.151 | 1.00 | 199.11 | C |
| ATOM | 8421 | O | GLY | B | 2109 | −16.913 | −12.500 | 61.705 | 1.00 | 199.11 | O |
| ATOM | 8422 | N | LYS | B | 2110 | −18.259 | −13.108 | 63.426 | 1.00 | 199.40 | N |
| ATOM | 8423 | CA | LYS | B | 2110 | −17.183 | −13.238 | 64.418 | 1.00 | 199.61 | C |
| ATOM | 8424 | C | LYS | B | 2110 | −16.252 | −14.438 | 64.097 | 1.00 | 199.82 | C |
| ATOM | 8425 | O | LYS | B | 2110 | −15.402 | −14.335 | 63.204 | 1.00 | 199.89 | O |
| ATOM | 8426 | CB | LYS | B | 2110 | −17.748 | −13.326 | 65.852 | 1.00 | 199.60 | C |
| ATOM | 8427 | CG | LYS | B | 2110 | −18.570 | −12.126 | 66.335 | 1.00 | 199.47 | C |
| ATOM | 8428 | CD | LYS | B | 2110 | −18.982 | −12.334 | 67.796 | 1.00 | 199.45 | C |
| ATOM | 8429 | CE | LYS | B | 2110 | −20.272 | −11.607 | 68.158 | 1.00 | 198.95 | C |
| ATOM | 8430 | NZ | LYS | B | 2110 | −20.086 | −10.152 | 68.377 | 1.00 | 198.70 | N |
| ATOM | 8431 | N | LYS | B | 2111 | −16.421 | −15.562 | 64.805 | 1.00 | 199.93 | N |
| ATOM | 8432 | CA | LYS | B | 2111 | −15.515 | −16.723 | 64.688 | 1.00 | 199.89 | C |
| ATOM | 8433 | C | LYS | B | 2111 | −15.747 | −17.547 | 63.425 | 1.00 | 199.85 | C |
| ATOM | 8434 | O | LYS | B | 2111 | −16.835 | −18.074 | 63.194 | 1.00 | 199.70 | O |
| ATOM | 8435 | CB | LYS | B | 2111 | −15.535 | −17.609 | 65.956 | 1.00 | 199.92 | C |
| ATOM | 8436 | CG | LYS | B | 2111 | −16.826 | −18.413 | 66.224 | 1.00 | 200.09 | C |
| ATOM | 8437 | CD | LYS | B | 2111 | −17.873 | −17.651 | 67.063 | 1.00 | 199.96 | C |
| ATOM | 8438 | CE | LYS | B | 2111 | −17.781 | −17.971 | 68.553 | 1.00 | 199.64 | C |
| ATOM | 8439 | NZ | LYS | B | 2111 | −16.683 | −17.233 | 69.234 | 1.00 | 199.52 | N |
| ATOM | 8440 | N | TRP | B | 2112 | −14.706 | −17.636 | 62.608 | 1.00 | 200.05 | N |
| ATOM | 8441 | CA | TRP | B | 2112 | −14.756 | −18.384 | 61.356 | 1.00 | 200.34 | C |
| ATOM | 8442 | C | TRP | B | 2112 | −14.172 | −19.773 | 61.576 | 1.00 | 200.66 | C |
| ATOM | 8443 | O | TRP | B | 2112 | −13.818 | −20.132 | 62.708 | 1.00 | 200.80 | O |
| ATOM | 8444 | CB | TRP | B | 2112 | −13.920 | −17.676 | 60.277 | 1.00 | 200.08 | C |
| ATOM | 8445 | CG | TRP | B | 2112 | −14.399 | −16.313 | 59.861 | 1.00 | 199.79 | C |
| ATOM | 8446 | CD1 | TRP | B | 2112 | −14.286 | −15.146 | 60.568 | 1.00 | 199.64 | C |
| ATOM | 8447 | CD2 | TRP | B | 2112 | −15.031 | −15.972 | 58.625 | 1.00 | 199.03 | C |
| ATOM | 8448 | NE1 | TRP | B | 2112 | −14.833 | −14.106 | 59.855 | 1.00 | 199.70 | N |
| ATOM | 8449 | CE2 | TRP | B | 2112 | −15.297 | −14.587 | 58.659 | 1.00 | 199.42 | C |
| ATOM | 8450 | CE3 | TRP | B | 2112 | −15.415 | −16.703 | 57.501 | 1.00 | 198.44 | C |
| ATOM | 8451 | CZ2 | TRP | B | 2112 | −15.924 | −13.923 | 57.607 | 1.00 | 199.43 | C |
| ATOM | 8452 | CZ3 | TRP | B | 2112 | −16.038 | −16.046 | 56.467 | 1.00 | 198.98 | C |
| ATOM | 8453 | CH2 | TRP | B | 2112 | −16.283 | −14.670 | 56.523 | 1.00 | 199.18 | C |
| ATOM | 8454 | N | GLN | B | 2113 | −14.102 | −20.550 | 60.491 | 1.00 | 200.96 | N |
| ATOM | 8455 | CA | GLN | B | 2113 | −13.141 | −21.663 | 60.351 | 1.00 | 201.29 | C |
| ATOM | 8456 | C | GLN | B | 2113 | −13.094 | −22.200 | 58.906 | 1.00 | 201.21 | C |
| ATOM | 8457 | O | GLN | B | 2113 | −13.937 | −21.845 | 58.072 | 1.00 | 201.19 | O |
| ATOM | 8458 | CB | GLN | B | 2113 | −13.363 | −22.793 | 61.393 | 1.00 | 201.42 | C |
| ATOM | 8459 | CG | GLN | B | 2113 | −14.376 | −23.871 | 61.019 | 1.00 | 201.93 | C |
| ATOM | 8460 | CD | GLN | B | 2113 | −15.788 | −23.337 | 60.925 | 1.00 | 202.79 | C |
| ATOM | 8461 | OE1 | GLN | B | 2113 | −16.098 | −22.483 | 60.080 | 1.00 | 202.86 | O |
| ATOM | 8462 | NE2 | GLN | B | 2113 | −16.660 | −23.840 | 61.795 | 1.00 | 203.17 | N |
| ATOM | 8463 | N | THR | B | 2114 | −12.093 | −23.042 | 58.637 | 1.00 | 201.07 | N |
| ATOM | 8464 | CA | THR | B | 2114 | −11.923 | −23.740 | 57.358 | 1.00 | 200.76 | C |
| ATOM | 8465 | C | THR | B | 2114 | −13.051 | −24.748 | 57.047 | 1.00 | 200.51 | C |
| ATOM | 8466 | O | THR | B | 2114 | −14.095 | −24.771 | 57.714 | 1.00 | 200.40 | O |
| ATOM | 8467 | CB | THR | B | 2114 | −10.538 | −24.458 | 57.287 | 1.00 | 200.81 | C |
| ATOM | 8468 | OG1 | THR | B | 2114 | −10.389 | −25.350 | 58.404 | 1.00 | 201.17 | O |
| ATOM | 8469 | CG2 | THR | B | 2114 | −9.394 | −23.452 | 57.290 | 1.00 | 200.66 | C |
| ATOM | 8470 | N | TYR | B | 2115 | −12.822 | −25.578 | 56.031 | 1.00 | 200.22 | N |
| ATOM | 8471 | CA | TYR | B | 2115 | −13.814 | −26.525 | 55.534 | 1.00 | 199.96 | C |
| ATOM | 8472 | C | TYR | B | 2115 | −13.206 | −27.892 | 55.285 | 1.00 | 200.05 | C |
| ATOM | 8473 | O | TYR | B | 2115 | −12.021 | −28.012 | 54.978 | 1.00 | 199.93 | O |
| ATOM | 8474 | CB | TYR | B | 2115 | −14.443 | −25.992 | 54.252 | 1.00 | 199.79 | C |
| ATOM | 8475 | CG | TYR | B | 2115 | −15.438 | −26.906 | 53.596 | 1.00 | 199.48 | C |
| ATOM | 8476 | CD1 | TYR | B | 2115 | −16.529 | −27.399 | 54.293 | 1.00 | 199.55 | C |
| ATOM | 8477 | CD2 | TYR | B | 2115 | −15.307 | −27.250 | 52.266 | 1.00 | 199.48 | C |
| ATOM | 8478 | CE1 | TYR | B | 2115 | −17.447 | −28.236 | 53.685 | 1.00 | 199.63 | C |
| ATOM | 8479 | CE2 | TYR | B | 2115 | −16.226 | −28.082 | 51.646 | 1.00 | 199.39 | C |
| ATOM | 8480 | CZ | TYR | B | 2115 | −17.291 | −28.569 | 52.358 | 1.00 | 199.29 | C |
| ATOM | 8481 | OH | TYR | B | 2115 | −18.197 | −29.397 | 51.742 | 1.00 | 199.28 | O |
| ATOM | 8482 | N | ARG | B | 2116 | −14.032 | −28.920 | 55.430 | 1.00 | 200.38 | N |
| ATOM | 8483 | CA | ARG | B | 2116 | −13.598 | −30.299 | 55.282 | 1.00 | 200.91 | C |
| ATOM | 8484 | C | ARG | B | 2116 | −14.681 | −31.150 | 54.638 | 1.00 | 201.08 | C |
| ATOM | 8485 | O | ARG | B | 2116 | −15.211 | −32.075 | 55.258 | 1.00 | 200.90 | O |
| ATOM | 8486 | CB | ARG | B | 2116 | −13.167 | −30.899 | 56.631 | 1.00 | 201.06 | C |
| ATOM | 8487 | CG | ARG | B | 2116 | −11.694 | −30.670 | 56.994 | 1.00 | 201.59 | C |
| ATOM | 8488 | CD | ARG | B | 2116 | −11.166 | −31.764 | 57.924 | 1.00 | 202.35 | C |
| ATOM | 8489 | NE | ARG | B | 2116 | −11.257 | −33.101 | 57.329 | 1.00 | 202.37 | N |
| ATOM | 8490 | CZ | ARG | B | 2116 | −11.924 | −34.129 | 57.852 | 1.00 | 202.05 | C |
| ATOM | 8491 | NH1 | ARG | B | 2116 | −12.568 | −34.020 | 59.009 | 1.00 | 201.60 | N |
| ATOM | 8492 | NH2 | ARG | B | 2116 | −11.932 | −35.285 | 57.215 | 1.00 | 202.23 | N |
| ATOM | 8493 | N | GLY | B | 2117 | −14.998 | −30.822 | 53.386 | 1.00 | 201.49 | N |
| ATOM | 8494 | CA | GLY | B | 2117 | −15.830 | −31.671 | 52.534 | 1.00 | 202.08 | C |
| ATOM | 8495 | C | GLY | B | 2117 | −15.576 | −33.135 | 52.830 | 1.00 | 202.50 | C |
| ATOM | 8496 | O | GLY | B | 2117 | −14.457 | −33.518 | 53.204 | 1.00 | 202.46 | O |
| ATOM | 8497 | N | ASN | B | 2118 | −16.605 | −33.959 | 52.653 | 1.00 | 202.94 | N |
| ATOM | 8498 | CA | ASN | B | 2118 | −16.589 | −35.301 | 53.231 | 1.00 | 203.37 | C |
| ATOM | 8499 | C | ASN | B | 2118 | −15.202 | −35.951 | 53.307 | 1.00 | 203.53 | C |
| ATOM | 8500 | O | ASN | B | 2118 | −14.416 | −35.923 | 52.343 | 1.00 | 203.38 | O |
| ATOM | 8501 | CB | ASN | B | 2118 | −17.722 | −36.250 | 52.723 | 1.00 | 203.47 | C |
| ATOM | 8502 | CG | ASN | B | 2118 | −17.778 | −36.402 | 51.198 | 1.00 | 203.85 | C |
| ATOM | 8503 | OD1 | ASN | B | 2118 | −16.752 | −36.578 | 50.539 | 1.00 | 204.01 | O |
| ATOM | 8504 | ND2 | ASN | B | 2118 | −18.995 | −36.314 | 50.634 | 1.00 | 204.64 | N |
| ATOM | 8505 | N | SER | B | 2119 | −14.915 | −36.436 | 54.520 | 1.00 | 203.82 | N |
| ATOM | 8506 | CA | SER | B | 2119 | −13.762 | −37.292 | 54.889 | 1.00 | 203.88 | C |
| ATOM | 8507 | C | SER | B | 2119 | −12.427 | −37.049 | 54.129 | 1.00 | 203.95 | C |
| ATOM | 8508 | O | SER | B | 2119 | −11.621 | −37.977 | 53.948 | 1.00 | 204.08 | O |
| ATOM | 8509 | CB | SER | B | 2119 | −14.169 | −38.797 | 54.884 | 1.00 | 203.81 | C |
| ATOM | 8510 | OG | SER | B | 2119 | −15.169 | −39.113 | 55.851 | 1.00 | 202.70 | O |
| ATOM | 8511 | N | THR | B | 2120 | −12.185 | −35.803 | 53.724 | 1.00 | 203.68 | N |
| ATOM | 8512 | CA | THR | B | 2120 | −11.021 | −35.491 | 52.903 | 1.00 | 203.60 | C |
| ATOM | 8513 | C | THR | B | 2120 | −9.678 | −35.783 | 53.638 | 1.00 | 203.44 | C |
| ATOM | 8514 | O | THR | B | 2120 | −9.116 | −36.878 | 53.513 | 1.00 | 203.21 | O |
| ATOM | 8515 | CB | THR | B | 2120 | −11.148 | −34.060 | 52.288 | 1.00 | 203.64 | C |
| ATOM | 8516 | OG1 | THR | B | 2120 | −10.020 | −33.765 | 51.448 | 1.00 | 203.91 | O |
| ATOM | 8517 | CG2 | THR | B | 2120 | −11.298 | −33.004 | 53.379 | 1.00 | 203.75 | C |
| ATOM | 8518 | N | GLY | B | 2121 | −9.192 | −34.821 | 54.415 | 1.00 | 203.34 | N |
| ATOM | 8519 | CA | GLY | B | 2121 | −7.939 | −34.965 | 55.143 | 1.00 | 203.13 | C |
| ATOM | 8520 | C | GLY | B | 2121 | −7.969 | −34.026 | 56.327 | 1.00 | 203.05 | C |
| ATOM | 8521 | O | GLY | B | 2121 | −8.594 | −34.332 | 57.341 | 1.00 | 203.09 | O |
| ATOM | 8522 | N | THR | B | 2122 | −7.313 | −32.875 | 56.184 | 1.00 | 202.87 | N |
| ATOM | 8523 | CA | THR | B | 2122 | −7.241 | −31.862 | 57.248 | 1.00 | 202.63 | C |
| ATOM | 8524 | C | THR | B | 2122 | −8.002 | −30.605 | 56.857 | 1.00 | 202.48 | C |
| ATOM | 8525 | O | THR | B | 2122 | −8.453 | −29.822 | 57.702 | 1.00 | 202.15 | O |
| ATOM | 8526 | CB | THR | B | 2122 | −5.767 | −31.468 | 57.564 | 1.00 | 202.66 | C |
| ATOM | 8527 | OG1 | THR | B | 2122 | −5.745 | −30.257 | 58.331 | 1.00 | 202.57 | O |
| ATOM | 8528 | CG2 | THR | B | 2122 | −4.948 | −31.262 | 56.279 | 1.00 | 202.35 | C |
| ATOM | 8529 | N | LEU | B | 2123 | −8.146 | −30.450 | 55.551 | 1.00 | 202.54 | N |
| ATOM | 8530 | CA | LEU | B | 2123 | −8.451 | −29.180 | 54.932 | 1.00 | 202.70 | C |
| ATOM | 8531 | C | LEU | B | 2123 | −8.924 | −29.508 | 53.533 | 1.00 | 202.80 | C |
| ATOM | 8532 | O | LEU | B | 2123 | −8.302 | −30.333 | 52.845 | 1.00 | 202.93 | O |
| ATOM | 8533 | CB | LEU | B | 2123 | −7.167 | −28.344 | 54.863 | 1.00 | 202.84 | C |
| ATOM | 8534 | CG | LEU | B | 2123 | −7.058 | −27.029 | 54.089 | 1.00 | 202.59 | C |
| ATOM | 8535 | CD1 | LEU | B | 2123 | −7.857 | −25.934 | 54.779 | 1.00 | 202.50 | C |
| ATOM | 8536 | CD2 | LEU | B | 2123 | −5.586 | −26.625 | 53.962 | 1.00 | 202.55 | C |
| ATOM | 8537 | N | MET | B | 2124 | −10.019 | −28.871 | 53.112 | 1.00 | 202.70 | N |
| ATOM | 8538 | CA | MET | B | 2124 | −10.654 | −29.208 | 51.831 | 1.00 | 202.51 | C |
| ATOM | 8539 | C | MET | B | 2124 | −10.139 | −28.372 | 50.669 | 1.00 | 202.41 | C |
| ATOM | 8540 | O | MET | B | 2124 | −10.554 | −27.223 | 50.468 | 1.00 | 202.39 | O |
| ATOM | 8541 | CB | MET | B | 2124 | −12.184 | −29.166 | 51.919 | 1.00 | 202.40 | C |
| ATOM | 8542 | CG | MET | B | 2124 | −12.901 | −29.604 | 50.643 | 1.00 | 202.16 | C |
| ATOM | 8543 | SD | MET | B | 2124 | −12.408 | −31.198 | 49.963 | 1.00 | 201.40 | S |
| ATOM | 8544 | CE | MET | B | 2124 | −13.557 | −31.317 | 48.583 | 1.00 | 201.95 | C |
| ATOM | 8545 | N | VAL | B | 2125 | −9.232 | −28.980 | 49.911 | 1.00 | 202.25 | N |
| ATOM | 8546 | CA | VAL | B | 2125 | −8.575 | −28.330 | 48.791 | 1.00 | 202.00 | C |
| ATOM | 8547 | C | VAL | B | 2125 | −9.434 | −28.446 | 47.530 | 1.00 | 201.89 | C |
| ATOM | 8548 | O | VAL | B | 2125 | −9.816 | −29.549 | 47.127 | 1.00 | 201.95 | O |
| ATOM | 8549 | CB | VAL | B | 2125 | −7.140 | −28.889 | 48.586 | 1.00 | 201.95 | C |
| ATOM | 8550 | CG1 | VAL | B | 2125 | −7.155 | −30.380 | 48.191 | 1.00 | 201.99 | C |
| ATOM | 8551 | CG2 | VAL | B | 2125 | −6.375 | −28.042 | 47.588 | 1.00 | 201.86 | C |
| ATOM | 8552 | N | PHE | B | 2126 | −9.745 | −27.301 | 46.928 | 1.00 | 201.69 | N |
| ATOM | 8553 | CA | PHE | B | 2126 | −10.638 | −27.250 | 45.775 | 1.00 | 201.65 | C |
| ATOM | 8554 | C | PHE | B | 2126 | −9.908 | −26.919 | 44.482 | 1.00 | 201.82 | C |
| ATOM | 8555 | O | PHE | B | 2126 | −9.050 | −26.037 | 44.462 | 1.00 | 201.86 | O |
| ATOM | 8556 | CB | PHE | B | 2126 | −11.746 | −26.230 | 46.010 | 1.00 | 201.45 | C |
| ATOM | 8557 | CG | PHE | B | 2126 | −12.870 | −26.737 | 46.862 | 1.00 | 201.14 | C |
| ATOM | 8558 | CD1 | PHE | B | 2126 | −13.746 | −27.710 | 46.377 | 1.00 | 201.36 | C |
| ATOM | 8559 | CD2 | PHE | B | 2126 | −13.074 | −26.226 | 48.134 | 1.00 | 200.42 | C |
| ATOM | 8560 | CE1 | PHE | B | 2126 | −14.799 | −28.178 | 47.153 | 1.00 | 201.04 | C |
| ATOM | 8561 | CE2 | PHE | B | 2126 | −14.122 | −26.679 | 48.911 | 1.00 | 200.57 | C |
| ATOM | 8562 | CZ | PHE | B | 2126 | −14.990 | −27.660 | 48.417 | 1.00 | 200.94 | C |
| ATOM | 8563 | N | PHE | B | 2127 | −10.268 | −27.621 | 43.406 | 1.00 | 201.99 | N |
| ATOM | 8564 | CA | PHE | B | 2127 | −9.625 | −27.438 | 42.103 | 1.00 | 202.21 | C |
| ATOM | 8565 | C | PHE | B | 2127 | −10.264 | −26.345 | 41.257 | 1.00 | 202.39 | C |
| ATOM | 8566 | O | PHE | B | 2127 | −11.465 | −26.403 | 40.970 | 1.00 | 202.75 | O |
| ATOM | 8567 | CB | PHE | B | 2127 | −9.585 | −28.753 | 41.339 | 1.00 | 202.16 | C |
| ATOM | 8568 | CG | PHE | B | 2127 | −8.394 | −29.580 | 41.665 | 1.00 | 202.24 | C |
| ATOM | 8569 | CD1 | PHE | B | 2127 | −7.204 | −29.405 | 40.961 | 1.00 | 202.35 | C |
| ATOM | 8570 | CD2 | PHE | B | 2127 | −8.442 | −30.512 | 42.698 | 1.00 | 202.40 | C |
| ATOM | 8571 | CE1 | PHE | B | 2127 | −6.078 | −30.166 | 41.263 | 1.00 | 202.65 | C |
| ATOM | 8572 | CE2 | PHE | B | 2127 | −7.325 | −31.280 | 43.016 | 1.00 | 202.77 | C |
| ATOM | 8573 | CZ | PHE | B | 2127 | −6.137 | −31.107 | 42.294 | 1.00 | 202.86 | C |
| ATOM | 8574 | N | GLY | B | 2128 | −9.441 | −25.378 | 40.840 | 1.00 | 202.32 | N |
| ATOM | 8575 | CA | GLY | B | 2128 | −9.905 | −24.128 | 40.212 | 1.00 | 202.20 | C |
| ATOM | 8576 | C | GLY | B | 2128 | −10.467 | −24.184 | 38.798 | 1.00 | 202.13 | C |
| ATOM | 8577 | O | GLY | B | 2128 | −11.450 | −24.889 | 38.529 | 1.00 | 202.32 | O |
| ATOM | 8578 | N | ASN | B | 2129 | −9.872 | −23.398 | 37.903 | 1.00 | 201.82 | N |
| ATOM | 8579 | CA | ASN | B | 2129 | −10.201 | −23.452 | 36.477 | 1.00 | 201.50 | C |
| ATOM | 8580 | C | ASN | B | 2129 | −8.966 | −23.897 | 35.730 | 1.00 | 201.31 | C |
| ATOM | 8581 | O | ASN | B | 2129 | −7.961 | −24.268 | 36.345 | 1.00 | 201.29 | O |
| ATOM | 8582 | CB | ASN | B | 2129 | −10.634 | −22.082 | 35.958 | 1.00 | 201.44 | C |
| ATOM | 8583 | CG | ASN | B | 2129 | −11.346 | −21.267 | 37.002 | 1.00 | 201.22 | C |
| ATOM | 8584 | OD1 | ASN | B | 2129 | −12.444 | −21.617 | 37.425 | 1.00 | 201.00 | O |
| ATOM | 8585 | ND2 | ASN | B | 2129 | −10.719 | −20.176 | 37.437 | 1.00 | 200.78 | N |
| ATOM | 8586 | N | VAL | B | 2130 | −9.030 | −23.870 | 34.406 | 1.00 | 200.96 | N |
| ATOM | 8587 | CA | VAL | B | 2130 | −7.856 | −24.202 | 33.617 | 1.00 | 200.64 | C |
| ATOM | 8588 | C | VAL | B | 2130 | −7.624 | −23.156 | 32.544 | 1.00 | 200.27 | C |
| ATOM | 8589 | O | VAL | B | 2130 | −6.584 | −23.157 | 31.881 | 1.00 | 200.29 | O |
| ATOM | 8590 | CB | VAL | B | 2130 | −7.968 | −25.593 | 32.970 | 1.00 | 200.79 | C |
| ATOM | 8591 | CG1 | VAL | B | 2130 | −6.572 | −26.243 | 32.871 | 1.00 | 200.81 | C |
| ATOM | 8592 | CG2 | VAL | B | 2130 | −8.961 | −26.491 | 33.750 | 1.00 | 200.99 | C |
| ATOM | 8593 | N | ASP | B | 2131 | −8.598 | −22.262 | 32.384 | 1.00 | 199.85 | N |
| ATOM | 8594 | CA | ASP | B | 2131 | −8.535 | −21.232 | 31.352 | 1.00 | 199.46 | C |
| ATOM | 8595 | C | ASP | B | 2131 | −9.386 | −20.004 | 31.676 | 1.00 | 198.91 | C |
| ATOM | 8596 | O | ASP | B | 2131 | −9.687 | −19.726 | 32.836 | 1.00 | 198.77 | O |
| ATOM | 8597 | CB | ASP | B | 2131 | −8.905 | −21.818 | 29.972 | 1.00 | 199.73 | C |
| ATOM | 8598 | CG | ASP | B | 2131 | −10.322 | −22.407 | 29.925 | 1.00 | 200.23 | C |
| ATOM | 8599 | OD1 | ASP | B | 2131 | −11.269 | −21.731 | 30.369 | 1.00 | 200.87 | O |
| ATOM | 8600 | OD2 | ASP | B | 2131 | −10.494 | −23.539 | 29.418 | 1.00 | 200.66 | O |
| ATOM | 8601 | N | SER | B | 2132 | −9.776 | −19.291 | 30.626 | 1.00 | 198.27 | N |
| ATOM | 8602 | CA | SER | B | 2132 | −10.502 | −18.048 | 30.739 | 1.00 | 197.78 | C |
| ATOM | 8603 | C | SER | B | 2132 | −11.993 | −18.258 | 30.536 | 1.00 | 197.66 | C |
| ATOM | 8604 | O | SER | B | 2132 | −12.729 | −17.301 | 30.321 | 1.00 | 197.46 | O |
| ATOM | 8605 | CB | SER | B | 2132 | −9.972 | −17.088 | 29.691 | 1.00 | 197.73 | C |
| ATOM | 8606 | OG | SER | B | 2132 | −8.590 | −17.318 | 29.475 | 1.00 | 197.63 | O |
| ATOM | 8607 | N | SER | B | 2133 | −12.425 | −19.518 | 30.597 | 1.00 | 197.76 | N |
| ATOM | 8608 | CA | SER | B | 2133 | −13.841 | −19.909 | 30.486 | 1.00 | 197.98 | C |
| ATOM | 8609 | C | SER | B | 2133 | −14.111 | −21.257 | 31.157 | 1.00 | 198.21 | C |
| ATOM | 8610 | O | SER | B | 2133 | −15.085 | −21.954 | 30.835 | 1.00 | 198.13 | O |
| ATOM | 8611 | CB | SER | B | 2133 | −14.260 | −19.976 | 29.026 | 1.00 | 197.85 | C |
| ATOM | 8612 | OG | SER | B | 2133 | −14.189 | −18.692 | 28.455 | 1.00 | 197.88 | O |
| ATOM | 8613 | N | GLY | B | 2134 | −13.226 | −21.615 | 32.087 | 1.00 | 198.56 | N |
| ATOM | 8614 | CA | GLY | B | 2134 | −13.308 | −22.872 | 32.817 | 1.00 | 198.90 | C |
| ATOM | 8615 | C | GLY | B | 2134 | −14.295 | −22.763 | 33.961 | 1.00 | 199.12 | C |
| ATOM | 8616 | O | GLY | B | 2134 | −13.897 | −22.580 | 35.118 | 1.00 | 199.45 | O |
| ATOM | 8617 | N | ILE | B | 2135 | −15.582 | −22.846 | 33.615 | 1.00 | 198.93 | N |
| ATOM | 8618 | CA | ILE | B | 2135 | −16.703 | −22.954 | 34.561 | 1.00 | 198.51 | C |
| ATOM | 8619 | C | ILE | B | 2135 | −16.674 | −24.270 | 35.361 | 1.00 | 198.51 | C |
| ATOM | 8620 | O | ILE | B | 2135 | −17.383 | −25.221 | 34.997 | 1.00 | 198.80 | O |
| ATOM | 8621 | CB | ILE | B | 2135 | −18.056 | −22.901 | 33.799 | 1.00 | 198.39 | C |
| ATOM | 8622 | CG1 | ILE | B | 2135 | −17.857 | −23.113 | 32.274 | 1.00 | 198.42 | C |
| ATOM | 8623 | CG2 | ILE | B | 2135 | −18.768 | −21.593 | 34.083 | 1.00 | 197.74 | C |
| ATOM | 8624 | CD1 | ILE | B | 2135 | −17.452 | −24.548 | 31.790 | 1.00 | 197.24 | C |
| ATOM | 8625 | N | LYS | B | 2136 | −15.871 | −24.347 | 36.431 | 1.00 | 198.08 | N |
| ATOM | 8626 | CA | LYS | B | 2136 | −15.709 | −25.634 | 37.132 | 1.00 | 197.68 | C |
| ATOM | 8627 | C | LYS | B | 2136 | −16.574 | −25.699 | 38.357 | 1.00 | 197.39 | C |
| ATOM | 8628 | O | LYS | B | 2136 | −16.101 | −25.554 | 39.477 | 1.00 | 197.53 | O |
| ATOM | 8629 | CB | LYS | B | 2136 | −14.247 | −25.977 | 37.447 | 1.00 | 197.61 | C |
| ATOM | 8630 | CG | LYS | B | 2136 | −13.857 | −27.463 | 37.183 | 1.00 | 197.91 | C |
| ATOM | 8631 | CD | LYS | B | 2136 | −14.254 | −28.052 | 35.751 | 1.00 | 198.26 | C |
| ATOM | 8632 | CE | LYS | B | 2136 | −13.836 | −27.229 | 34.455 | 1.00 | 196.85 | C |
| ATOM | 8633 | NZ | LYS | B | 2136 | −12.412 | −27.281 | 33.974 | 1.00 | 194.58 | N |
| ATOM | 8634 | N | HIS | B | 2137 | −17.861 | −25.893 | 38.089 | 1.00 | 197.16 | N |
| ATOM | 8635 | CA | HIS | B | 2137 | −18.925 | −26.065 | 39.072 | 1.00 | 196.77 | C |
| ATOM | 8636 | C | HIS | B | 2137 | −18.500 | −27.043 | 40.163 | 1.00 | 196.20 | C |
| ATOM | 8637 | O | HIS | B | 2137 | −18.605 | −28.261 | 39.996 | 1.00 | 196.39 | O |
| ATOM | 8638 | CB | HIS | B | 2137 | −20.199 | −26.553 | 38.341 | 1.00 | 197.05 | C |
| ATOM | 8639 | CG | HIS | B | 2137 | −21.392 | −26.766 | 39.227 | 1.00 | 197.50 | C |
| ATOM | 8640 | ND1 | HIS | B | 2137 | −22.524 | −27.422 | 38.789 | 1.00 | 197.98 | N |
| ATOM | 8641 | CD2 | HIS | B | 2137 | −21.631 | −26.422 | 40.517 | 1.00 | 197.76 | C |
| ATOM | 8642 | CE1 | HIS | B | 2137 | −23.411 | −27.466 | 39.769 | 1.00 | 198.49 | C |
| ATOM | 8643 | NE2 | HIS | B | 2137 | −22.892 | −26.871 | 40.831 | 1.00 | 198.02 | N |
| ATOM | 8644 | N | ASN | B | 2138 | −17.986 | −26.496 | 41.262 | 1.00 | 195.23 | N |
| ATOM | 8645 | CA | ASN | B | 2138 | −17.653 | −27.285 | 42.437 | 1.00 | 194.17 | C |
| ATOM | 8646 | C | ASN | B | 2138 | −18.897 | −27.472 | 43.286 | 1.00 | 194.13 | C |
| ATOM | 8647 | O | ASN | B | 2138 | −19.655 | −26.506 | 43.467 | 1.00 | 194.09 | O |
| ATOM | 8648 | CB | ASN | B | 2138 | −16.581 | −26.574 | 43.253 | 1.00 | 193.62 | C |
| ATOM | 8649 | CG | ASN | B | 2138 | −15.256 | −27.262 | 43.183 | 1.00 | 192.13 | C |
| ATOM | 8650 | OD1 | ASN | B | 2138 | −15.164 | −28.483 | 43.326 | 1.00 | 190.24 | O |
| ATOM | 8651 | ND2 | ASN | B | 2138 | −14.208 | −26.485 | 42.976 | 1.00 | 190.85 | N |
| ATOM | 8652 | N | ILE | B | 2139 | −19.130 | −28.701 | 43.777 | 1.00 | 193.76 | N |
| ATOM | 8653 | CA | ILE | B | 2139 | −20.186 | −28.938 | 44.801 | 1.00 | 193.12 | C |
| ATOM | 8654 | C | ILE | B | 2139 | −19.663 | −29.325 | 46.182 | 1.00 | 192.38 | C |
| ATOM | 8655 | O | ILE | B | 2139 | −18.673 | −30.047 | 46.323 | 1.00 | 191.87 | O |
| ATOM | 8656 | CB | ILE | B | 2139 | −21.414 | −29.861 | 44.358 | 1.00 | 193.30 | C |
| ATOM | 8657 | CG1 | ILE | B | 2139 | −20.974 | −31.236 | 43.825 | 1.00 | 193.28 | C |
| ATOM | 8658 | CG2 | ILE | B | 2139 | −22.374 | −29.103 | 43.398 | 1.00 | 193.33 | C |
| ATOM | 8659 | CD1 | ILE | B | 2139 | −22.122 | −32.278 | 43.715 | 1.00 | 193.05 | C |
| ATOM | 8660 | N | PHE | B | 2140 | −20.350 | −28.794 | 47.186 | 1.00 | 192.00 | N |
| ATOM | 8661 | CA | PHE | B | 2140 | −20.010 | −29.007 | 48.583 | 1.00 | 191.90 | C |
| ATOM | 8662 | C | PHE | B | 2140 | −20.635 | −30.329 | 49.060 | 1.00 | 191.67 | C |
| ATOM | 8663 | O | PHE | B | 2140 | −21.858 | −30.418 | 49.280 | 1.00 | 191.67 | O |
| ATOM | 8664 | CB | PHE | B | 2140 | −20.432 | −27.799 | 49.464 | 1.00 | 191.85 | C |
| ATOM | 8665 | CG | PHE | B | 2140 | −19.813 | −26.460 | 49.047 | 1.00 | 191.46 | C |
| ATOM | 8666 | CD1 | PHE | B | 2140 | −18.491 | −26.377 | 48.604 | 1.00 | 190.76 | C |
| ATOM | 8667 | CD2 | PHE | B | 2140 | −20.555 | −25.283 | 49.130 | 1.00 | 190.43 | C |
| ATOM | 8668 | CE1 | PHE | B | 2140 | −17.943 | −25.155 | 48.233 | 1.00 | 189.85 | C |
| ATOM | 8669 | CE2 | PHE | B | 2140 | −20.006 | −24.062 | 48.764 | 1.00 | 189.76 | C |
| ATOM | 8670 | CZ | PHE | B | 2140 | −18.702 | −23.997 | 48.320 | 1.00 | 189.91 | C |
| ATOM | 8671 | N | ASN | B | 2141 | −19.759 | −31.334 | 49.213 | 1.00 | 191.16 | N |
| ATOM | 8672 | CA | ASN | B | 2141 | −20.106 | −32.761 | 49.392 | 1.00 | 190.38 | C |
| ATOM | 8673 | C | ASN | B | 2141 | −21.091 | −33.028 | 50.536 | 1.00 | 189.32 | C |
| ATOM | 8674 | O | ASN | B | 2141 | −22.140 | −33.661 | 50.328 | 1.00 | 188.75 | O |
| ATOM | 8675 | CB | ASN | B | 2141 | −18.815 | −33.605 | 49.532 | 1.00 | 190.78 | C |
| ATOM | 8676 | CG | ASN | B | 2141 | −18.302 | −34.178 | 48.187 | 1.00 | 191.63 | C |
| ATOM | 8677 | OD1 | ASN | B | 2141 | −17.798 | −35.313 | 48.133 | 1.00 | 192.23 | O |
| ATOM | 8678 | ND2 | ASN | B | 2141 | −18.424 | −33.396 | 47.110 | 1.00 | 192.58 | N |
| ATOM | 8679 | N | PRO | B | 2142 | −20.725 | −32.587 | 51.751 | 1.00 | 188.62 | N |
| ATOM | 8680 | CA | PRO | B | 2142 | −21.730 | −32.171 | 52.691 | 1.00 | 188.21 | C |
| ATOM | 8681 | C | PRO | B | 2142 | −21.937 | −30.669 | 52.461 | 1.00 | 187.64 | C |
| ATOM | 8682 | O | PRO | B | 2142 | −20.997 | −29.974 | 52.063 | 1.00 | 187.24 | O |
| ATOM | 8683 | CB | PRO | B | 2142 | −21.078 | −32.451 | 54.054 | 1.00 | 188.26 | C |
| ATOM | 8684 | CG | PRO | B | 2142 | −19.651 | −32.897 | 53.762 | 1.00 | 188.28 | C |
| ATOM | 8685 | CD | PRO | B | 2142 | −19.386 | −32.499 | 52.355 | 1.00 | 188.61 | C |
| ATOM | 8686 | N | PRO | B | 2143 | −23.154 | −30.161 | 52.719 | 1.00 | 187.28 | N |
| ATOM | 8687 | CA | PRO | B | 2143 | −23.470 | −28.815 | 52.245 | 1.00 | 187.10 | C |
| ATOM | 8688 | C | PRO | B | 2143 | −22.815 | −27.809 | 53.171 | 1.00 | 187.06 | C |
| ATOM | 8689 | O | PRO | B | 2143 | −22.056 | −28.218 | 54.039 | 1.00 | 187.22 | O |
| ATOM | 8690 | CB | PRO | B | 2143 | −24.997 | −28.750 | 52.356 | 1.00 | 187.05 | C |
| ATOM | 8691 | CG | PRO | B | 2143 | −25.441 | −30.088 | 52.944 | 1.00 | 186.97 | C |
| ATOM | 8692 | CD | PRO | B | 2143 | −24.252 | −30.753 | 53.506 | 1.00 | 187.11 | C |
| ATOM | 8693 | N | ILE | B | 2144 | −23.068 | −26.516 | 52.996 | 1.00 | 186.94 | N |
| ATOM | 8694 | CA | ILE | B | 2144 | −22.625 | −25.541 | 53.994 | 1.00 | 186.89 | C |
| ATOM | 8695 | C | ILE | B | 2144 | −23.757 | −24.600 | 54.341 | 1.00 | 186.89 | C |
| ATOM | 8696 | O | ILE | B | 2144 | −24.450 | −24.116 | 53.453 | 1.00 | 186.86 | O |
| ATOM | 8697 | CB | ILE | B | 2144 | −21.370 | −24.729 | 53.550 | 1.00 | 186.93 | C |
| ATOM | 8698 | CG1 | ILE | B | 2144 | −20.132 | −25.632 | 53.447 | 1.00 | 187.08 | C |
| ATOM | 8699 | CG2 | ILE | B | 2144 | −21.101 | −23.575 | 54.521 | 1.00 | 186.66 | C |
| ATOM | 8700 | CD1 | ILE | B | 2144 | −18.915 | −24.987 | 52.802 | 1.00 | 186.93 | C |
| ATOM | 8701 | N | ILE | B | 2145 | −23.951 | −24.366 | 55.635 | 1.00 | 187.01 | N |
| ATOM | 8702 | CA | ILE | B | 2145 | −24.850 | −23.319 | 56.106 | 1.00 | 187.29 | C |
| ATOM | 8703 | C | ILE | B | 2145 | −24.011 | −22.147 | 56.594 | 1.00 | 187.42 | C |
| ATOM | 8704 | O | ILE | B | 2145 | −23.263 | −22.284 | 57.561 | 1.00 | 187.38 | O |
| ATOM | 8705 | CB | ILE | B | 2145 | −25.797 | −23.813 | 57.229 | 1.00 | 187.30 | C |
| ATOM | 8706 | CG1 | ILE | B | 2145 | −25.048 | −24.697 | 58.234 | 1.00 | 188.23 | C |
| ATOM | 8707 | CG2 | ILE | B | 2145 | −26.978 | −24.582 | 56.655 | 1.00 | 186.98 | C |
| ATOM | 8708 | CD1 | ILE | B | 2145 | −24.494 | −23.950 | 59.474 | 1.00 | 189.58 | C |
| ATOM | 8709 | N | ALA | B | 2146 | −24.116 | −21.010 | 55.908 | 1.00 | 187.74 | N |
| ATOM | 8710 | CA | ALA | B | 2146 | −23.351 | −19.803 | 56.265 | 1.00 | 188.12 | C |
| ATOM | 8711 | C | ALA | B | 2146 | −24.108 | −18.521 | 55.933 | 1.00 | 188.40 | C |
| ATOM | 8712 | O | ALA | B | 2146 | −25.202 | −18.570 | 55.377 | 1.00 | 188.51 | O |
| ATOM | 8713 | CB | ALA | B | 2146 | −21.979 | −19.805 | 55.576 | 1.00 | 187.86 | C |
| ATOM | 8714 | N | ARG | B | 2147 | −23.531 | −17.381 | 56.305 | 1.00 | 188.83 | N |
| ATOM | 8715 | CA | ARG | B | 2147 | −23.965 | −16.082 | 55.792 | 1.00 | 189.30 | C |
| ATOM | 8716 | C | ARG | B | 2147 | −22.829 | −15.485 | 54.954 | 1.00 | 189.48 | C |
| ATOM | 8717 | O | ARG | B | 2147 | −23.053 | −14.777 | 53.962 | 1.00 | 189.53 | O |
| ATOM | 8718 | CB | ARG | B | 2147 | −24.349 | −15.141 | 56.935 | 1.00 | 189.31 | C |
| ATOM | 8719 | CG | ARG | B | 2147 | −24.859 | −13.782 | 56.483 | 1.00 | 189.91 | C |
| ATOM | 8720 | CD | ARG | B | 2147 | −25.083 | −12.901 | 57.679 | 1.00 | 191.71 | C |
| ATOM | 8721 | NE | ARG | B | 2147 | −25.321 | −11.499 | 57.337 | 1.00 | 193.35 | N |
| ATOM | 8722 | CZ | ARG | B | 2147 | −26.410 | −10.813 | 57.688 | 1.00 | 194.49 | C |
| ATOM | 8723 | NH1 | ARG | B | 2147 | −27.382 | −11.400 | 58.388 | 1.00 | 195.26 | N |
| ATOM | 8724 | NH2 | ARG | B | 2147 | −26.531 | −9.532 | 57.345 | 1.00 | 194.64 | N |
| ATOM | 8725 | N | TYR | B | 2148 | −21.606 | −15.810 | 55.355 | 1.00 | 189.63 | N |
| ATOM | 8726 | CA | TYR | B | 2148 | −20.411 | −15.312 | 54.707 | 1.00 | 189.64 | C |
| ATOM | 8727 | C | TYR | B | 2148 | −19.607 | −16.474 | 54.150 | 1.00 | 189.66 | C |
| ATOM | 8728 | O | TYR | B | 2148 | −19.468 | −17.507 | 54.821 | 1.00 | 189.84 | O |
| ATOM | 8729 | CB | TYR | B | 2148 | −19.556 | −14.570 | 55.728 | 1.00 | 189.75 | C |
| ATOM | 8730 | CG | TYR | B | 2148 | −20.247 | −13.417 | 56.402 | 1.00 | 189.74 | C |
| ATOM | 8731 | CD1 | TYR | B | 2148 | −21.112 | −13.627 | 57.472 | 1.00 | 189.38 | C |
| ATOM | 8732 | CD2 | TYR | B | 2148 | −20.019 | −12.109 | 55.975 | 1.00 | 190.08 | C |
| ATOM | 8733 | CE1 | TYR | B | 2148 | −21.740 | −12.570 | 58.090 | 1.00 | 189.97 | C |
| ATOM | 8734 | CE2 | TYR | B | 2148 | −20.639 | −11.038 | 56.586 | 1.00 | 190.37 | C |
| ATOM | 8735 | CZ | TYR | B | 2148 | −21.503 | −11.273 | 57.644 | 1.00 | 190.38 | C |
| ATOM | 8736 | OH | TYR | B | 2148 | −22.122 | −10.204 | 58.260 | 1.00 | 190.46 | O |
| ATOM | 8737 | N | ILE | B | 2149 | −19.092 | −16.309 | 52.928 | 1.00 | 189.48 | N |
| ATOM | 8738 | CA | ILE | B | 2149 | −18.071 | −17.214 | 52.383 | 1.00 | 189.28 | C |
| ATOM | 8739 | C | ILE | B | 2149 | −16.829 | −16.437 | 51.942 | 1.00 | 189.70 | C |
| ATOM | 8740 | O | ILE | B | 2149 | −16.912 | −15.368 | 51.320 | 1.00 | 189.54 | O |
| ATOM | 8741 | CB | ILE | B | 2149 | −18.561 | −18.103 | 51.208 | 1.00 | 188.91 | C |
| ATOM | 8742 | CG1 | ILE | B | 2149 | −20.069 | −18.308 | 51.234 | 1.00 | 188.86 | C |
| ATOM | 8743 | CG2 | ILE | B | 2149 | −17.882 | −19.456 | 51.248 | 1.00 | 188.04 | C |
| ATOM | 8744 | CD1 | ILE | B | 2149 | −20.876 | −17.093 | 50.835 | 1.00 | 189.24 | C |
| ATOM | 8745 | N | ARG | B | 2150 | −15.677 | −16.983 | 52.312 | 1.00 | 190.22 | N |
| ATOM | 8746 | CA | ARG | B | 2150 | −14.398 | −16.501 | 51.842 | 1.00 | 190.73 | C |
| ATOM | 8747 | C | ARG | B | 2150 | −13.876 | −17.502 | 50.833 | 1.00 | 191.03 | C |
| ATOM | 8748 | O | ARG | B | 2150 | −14.030 | −18.719 | 50.991 | 1.00 | 191.04 | O |
| ATOM | 8749 | CB | ARG | B | 2150 | −13.401 | −16.409 | 52.988 | 1.00 | 190.77 | C |
| ATOM | 8750 | CG | ARG | B | 2150 | −13.421 | −15.128 | 53.773 | 1.00 | 191.40 | C |
| ATOM | 8751 | CD | ARG | B | 2150 | −12.696 | −15.357 | 55.090 | 1.00 | 192.61 | C |
| ATOM | 8752 | NE | ARG | B | 2150 | −12.397 | −14.113 | 55.792 | 1.00 | 193.62 | N |
| ATOM | 8753 | CZ | ARG | B | 2150 | −12.037 | −14.047 | 57.069 | 1.00 | 194.23 | C |
| ATOM | 8754 | NH1 | ARG | B | 2150 | −11.941 | −15.161 | 57.788 | 1.00 | 194.30 | N |
| ATOM | 8755 | NH2 | ARG | B | 2150 | −11.780 | −12.866 | 57.631 | 1.00 | 194.56 | N |
| ATOM | 8756 | N | LEU | B | 2151 | −13.261 | −16.984 | 49.786 | 1.00 | 191.39 | N |
| ATOM | 8757 | CA | LEU | B | 2151 | −12.513 | −17.822 | 48.892 | 1.00 | 191.73 | C |
| ATOM | 8758 | C | LEU | B | 2151 | −11.069 | −17.372 | 48.984 | 1.00 | 191.80 | C |
| ATOM | 8759 | O | LEU | B | 2151 | −10.781 | −16.180 | 48.874 | 1.00 | 191.79 | O |
| ATOM | 8760 | CB | LEU | B | 2151 | −13.032 | −17.670 | 47.474 | 1.00 | 191.84 | C |
| ATOM | 8761 | CG | LEU | B | 2151 | −12.433 | −18.663 | 46.479 | 1.00 | 192.98 | C |
| ATOM | 8762 | CD1 | LEU | B | 2151 | −13.382 | −18.846 | 45.310 | 1.00 | 194.42 | C |
| ATOM | 8763 | CD2 | LEU | B | 2151 | −11.035 | −18.247 | 45.983 | 1.00 | 194.03 | C |
| ATOM | 8764 | N | HIS | B | 2152 | −10.172 | −18.333 | 49.192 | 1.00 | 191.91 | N |
| ATOM | 8765 | CA | HIS | B | 2152 | −8.741 | −18.059 | 49.307 | 1.00 | 191.99 | C |
| ATOM | 8766 | C | HIS | B | 2152 | −7.966 | −18.830 | 48.217 | 1.00 | 192.05 | C |
| ATOM | 8767 | O | HIS | B | 2152 | −8.054 | −20.051 | 48.161 | 1.00 | 192.14 | O |
| ATOM | 8768 | CB | HIS | B | 2152 | −8.223 | −18.478 | 50.699 | 1.00 | 191.97 | C |
| ATOM | 8769 | CG | HIS | B | 2152 | −8.921 | −17.826 | 51.861 | 1.00 | 191.65 | C |
| ATOM | 8770 | ND1 | HIS | B | 2152 | −8.249 | −17.078 | 52.805 | 1.00 | 191.37 | N |
| ATOM | 8771 | CD2 | HIS | B | 2152 | −10.216 | −17.849 | 52.259 | 1.00 | 191.60 | C |
| ATOM | 8772 | CE1 | HIS | B | 2152 | −9.101 | −16.650 | 53.718 | 1.00 | 191.38 | C |
| ATOM | 8773 | NE2 | HIS | B | 2152 | −10.302 | −17.104 | 53.410 | 1.00 | 191.44 | N |
| ATOM | 8774 | N | PRO | B | 2153 | −7.210 | −18.132 | 47.351 | 1.00 | 192.08 | N |
| ATOM | 8775 | CA | PRO | B | 2153 | −6.380 | −18.831 | 46.360 | 1.00 | 192.33 | C |
| ATOM | 8776 | C | PRO | B | 2153 | −5.255 | −19.716 | 46.954 | 1.00 | 192.61 | C |
| ATOM | 8777 | O | PRO | B | 2153 | −4.644 | −19.329 | 47.950 | 1.00 | 192.61 | O |
| ATOM | 8778 | CB | PRO | B | 2153 | −5.765 | −17.675 | 45.565 | 1.00 | 192.40 | C |
| ATOM | 8779 | CG | PRO | B | 2153 | −5.807 | −16.514 | 46.491 | 1.00 | 192.18 | C |
| ATOM | 8780 | CD | PRO | B | 2153 | −7.094 | −16.671 | 47.215 | 1.00 | 191.98 | C |
| ATOM | 8781 | N | THR | B | 2154 | −4.996 | −20.881 | 46.340 | 1.00 | 192.99 | N |
| ATOM | 8782 | CA | THR | B | 2154 | −3.842 | −21.771 | 46.677 | 1.00 | 193.41 | C |
| ATOM | 8783 | C | THR | B | 2154 | −2.870 | −21.950 | 45.479 | 1.00 | 193.56 | C |
| ATOM | 8784 | O | THR | B | 2154 | −1.770 | −22.509 | 45.606 | 1.00 | 193.34 | O |
| ATOM | 8785 | CB | THR | B | 2154 | −4.290 | −23.174 | 47.217 | 1.00 | 193.52 | C |
| ATOM | 8786 | OG1 | THR | B | 2154 | −5.624 | −23.101 | 47.734 | 1.00 | 193.59 | O |
| ATOM | 8787 | CG2 | THR | B | 2154 | −3.323 | −23.716 | 48.311 | 1.00 | 193.26 | C |
| ATOM | 8788 | N | HIS | B | 2155 | −3.315 | −21.509 | 44.308 | 1.00 | 193.88 | N |
| ATOM | 8789 | CA | HIS | B | 2155 | −2.414 | −21.150 | 43.225 | 1.00 | 194.33 | C |
| ATOM | 8790 | C | HIS | B | 2155 | −3.127 | −20.455 | 42.054 | 1.00 | 194.22 | C |
| ATOM | 8791 | O | HIS | B | 2155 | −4.296 | −20.715 | 41.759 | 1.00 | 194.01 | O |
| ATOM | 8792 | CB | HIS | B | 2155 | −1.521 | −22.312 | 42.778 | 1.00 | 194.61 | C |
| ATOM | 8793 | CG | HIS | B | 2155 | −0.193 | −21.864 | 42.240 | 1.00 | 196.38 | C |
| ATOM | 8794 | ND1 | HIS | B | 2155 | 0.833 | −21.428 | 43.054 | 1.00 | 197.56 | N |
| ATOM | 8795 | CD2 | HIS | B | 2155 | 0.270 | −21.762 | 40.968 | 1.00 | 197.74 | C |
| ATOM | 8796 | CE1 | HIS | B | 2155 | 1.872 | −21.088 | 42.311 | 1.00 | 197.83 | C |
| ATOM | 8797 | NE2 | HIS | B | 2155 | 1.558 | −21.281 | 41.041 | 1.00 | 198.12 | N |
| ATOM | 8798 | N | TYR | B | 2156 | −2.388 | −19.572 | 41.401 | 1.00 | 194.32 | N |
| ATOM | 8799 | CA | TYR | B | 2156 | −2.920 | −18.646 | 40.421 | 1.00 | 194.57 | C |
| ATOM | 8800 | C | TYR | B | 2156 | −2.150 | −18.777 | 39.116 | 1.00 | 194.58 | C |
| ATOM | 8801 | O | TYR | B | 2156 | −1.198 | −19.558 | 39.022 | 1.00 | 194.53 | O |
| ATOM | 8802 | CB | TYR | B | 2156 | −2.781 | −17.222 | 40.961 | 1.00 | 194.85 | C |
| ATOM | 8803 | CG | TYR | B | 2156 | −1.590 | −17.073 | 41.875 | 1.00 | 195.42 | C |
| ATOM | 8804 | CD1 | TYR | B | 2156 | −0.288 | −17.164 | 41.371 | 1.00 | 195.90 | C |
| ATOM | 8805 | CD2 | TYR | B | 2156 | −1.756 | −16.877 | 43.250 | 1.00 | 196.26 | C |
| ATOM | 8806 | CE1 | TYR | B | 2156 | 0.828 | −17.052 | 42.204 | 1.00 | 196.27 | C |
| ATOM | 8807 | CE2 | TYR | B | 2156 | −0.644 | −16.758 | 44.102 | 1.00 | 196.91 | C |
| ATOM | 8808 | CZ | TYR | B | 2156 | 0.652 | −16.847 | 43.565 | 1.00 | 196.36 | C |
| ATOM | 8809 | OH | TYR | B | 2156 | 1.771 | −16.735 | 44.371 | 1.00 | 195.82 | O |
| ATOM | 8810 | N | SER | B | 2157 | −2.569 | −18.005 | 38.115 | 1.00 | 194.66 | N |
| ATOM | 8811 | CA | SER | B | 2157 | −1.920 | −17.984 | 36.809 | 1.00 | 194.64 | C |
| ATOM | 8812 | C | SER | B | 2157 | −0.957 | −16.825 | 36.756 | 1.00 | 194.69 | C |
| ATOM | 8813 | O | SER | B | 2157 | 0.232 | −16.972 | 37.017 | 1.00 | 194.62 | O |
| ATOM | 8814 | CB | SER | B | 2157 | −2.951 | −17.804 | 35.700 | 1.00 | 194.57 | C |
| ATOM | 8815 | OG | SER | B | 2157 | −3.970 | −18.775 | 35.775 | 1.00 | 194.43 | O |
| ATOM | 8816 | N | ILE | B | 2158 | −1.489 | −15.666 | 36.408 | 1.00 | 194.90 | N |
| ATOM | 8817 | CA | ILE | B | 2158 | −0.704 | −14.458 | 36.363 | 1.00 | 195.43 | C |
| ATOM | 8818 | C | ILE | B | 2158 | −1.097 | −13.614 | 37.568 | 1.00 | 195.57 | C |
| ATOM | 8819 | O | ILE | B | 2158 | −0.249 | −13.008 | 38.222 | 1.00 | 195.78 | O |
| ATOM | 8820 | CB | ILE | B | 2158 | −0.918 | −13.739 | 35.024 | 1.00 | 195.56 | C |
| ATOM | 8821 | CG1 | ILE | B | 2158 | −0.516 | −14.690 | 33.891 | 1.00 | 196.15 | C |
| ATOM | 8822 | CG2 | ILE | B | 2158 | −0.109 | −12.424 | 34.952 | 1.00 | 195.96 | C |
| ATOM | 8823 | CD1 | ILE | B | 2158 | −1.307 | −14.555 | 32.619 | 1.00 | 196.98 | C |
| ATOM | 8824 | N | ARG | B | 2159 | −2.390 | −13.601 | 37.862 | 1.00 | 195.70 | N |
| ATOM | 8825 | CA | ARG | B | 2159 | −2.910 | −13.009 | 39.080 | 1.00 | 195.70 | C |
| ATOM | 8826 | C | ARG | B | 2159 | −3.970 | −13.943 | 39.619 | 1.00 | 195.97 | C |
| ATOM | 8827 | O | ARG | B | 2159 | −4.643 | −14.635 | 38.846 | 1.00 | 196.02 | O |
| ATOM | 8828 | CB | ARG | B | 2159 | −3.520 | −11.643 | 38.790 | 1.00 | 195.44 | C |
| ATOM | 8829 | CG | ARG | B | 2159 | −2.500 | −10.534 | 38.735 | 1.00 | 195.40 | C |
| ATOM | 8830 | CD | ARG | B | 2159 | −2.636 | −9.716 | 37.475 | 1.00 | 195.49 | C |
| ATOM | 8831 | NE | ARG | B | 2159 | −3.975 | −9.150 | 37.322 | 1.00 | 195.83 | N |
| ATOM | 8832 | CZ | ARG | B | 2159 | −4.400 | −8.511 | 36.234 | 1.00 | 196.08 | C |
| ATOM | 8833 | NH1 | ARG | B | 2159 | −3.585 | −8.357 | 35.189 | 1.00 | 196.37 | N |
| ATOM | 8834 | NH2 | ARG | B | 2159 | −5.642 | −8.026 | 36.186 | 1.00 | 195.42 | N |
| ATOM | 8835 | N | SER | B | 2160 | −4.096 | −13.982 | 40.945 | 1.00 | 196.27 | N |
| ATOM | 8836 | CA | SER | B | 2160 | −5.269 | −14.567 | 41.595 | 1.00 | 196.44 | C |
| ATOM | 8837 | C | SER | B | 2160 | −6.506 | −13.873 | 40.988 | 1.00 | 196.52 | C |
| ATOM | 8838 | O | SER | B | 2160 | −6.804 | −12.717 | 41.332 | 1.00 | 196.85 | O |
| ATOM | 8839 | CB | SER | B | 2160 | −5.195 | −14.367 | 43.120 | 1.00 | 196.45 | C |
| ATOM | 8840 | OG | SER | B | 2160 | −3.931 | −14.758 | 43.648 | 1.00 | 196.09 | O |
| ATOM | 8841 | N | THR | B | 2161 | −7.187 | −14.564 | 40.061 | 1.00 | 196.20 | N |
| ATOM | 8842 | CA | THR | B | 2161 | −8.214 | −13.949 | 39.188 | 1.00 | 195.72 | C |
| ATOM | 8843 | C | THR | B | 2161 | −9.503 | −14.781 | 39.047 | 1.00 | 195.44 | C |
| ATOM | 8844 | O | THR | B | 2161 | −9.481 | −15.936 | 38.606 | 1.00 | 195.37 | O |
| ATOM | 8845 | CB | THR | B | 2161 | −7.625 | −13.607 | 37.793 | 1.00 | 195.71 | C |
| ATOM | 8846 | OG1 | THR | B | 2161 | −6.584 | −12.633 | 37.941 | 1.00 | 195.39 | O |
| ATOM | 8847 | CG2 | THR | B | 2161 | −8.694 | −13.062 | 36.862 | 1.00 | 195.62 | C |
| ATOM | 8848 | N | LEU | B | 2162 | −10.630 | −14.169 | 39.395 | 1.00 | 195.01 | N |
| ATOM | 8849 | CA | LEU | B | 2162 | −11.848 | −14.927 | 39.579 | 1.00 | 194.60 | C |
| ATOM | 8850 | C | LEU | B | 2162 | −13.126 | −14.208 | 39.193 | 1.00 | 194.78 | C |
| ATOM | 8851 | O | LEU | B | 2162 | −13.243 | −12.988 | 39.270 | 1.00 | 194.54 | O |
| ATOM | 8852 | CB | LEU | B | 2162 | −11.935 | −15.384 | 41.030 | 1.00 | 194.37 | C |
| ATOM | 8853 | CG | LEU | B | 2162 | −13.286 | −15.833 | 41.570 | 1.00 | 193.67 | C |
| ATOM | 8854 | CD1 | LEU | B | 2162 | −13.484 | −17.296 | 41.329 | 1.00 | 193.10 | C |
| ATOM | 8855 | CD2 | LEU | B | 2162 | −13.345 | −15.547 | 43.037 | 1.00 | 193.66 | C |
| ATOM | 8856 | N | ARG | B | 2163 | −14.073 | −15.022 | 38.762 | 1.00 | 195.27 | N |
| ATOM | 8857 | CA | ARG | B | 2163 | −15.454 | −14.646 | 38.562 | 1.00 | 196.12 | C |
| ATOM | 8858 | C | ARG | B | 2163 | −16.237 | −15.925 | 38.896 | 1.00 | 196.63 | C |
| ATOM | 8859 | O | ARG | B | 2163 | −15.807 | −17.032 | 38.525 | 1.00 | 196.88 | O |
| ATOM | 8860 | CB | ARG | B | 2163 | −15.685 | −14.191 | 37.116 | 1.00 | 196.23 | C |
| ATOM | 8861 | CG | ARG | B | 2163 | −15.780 | −15.328 | 36.083 | 1.00 | 196.60 | C |
| ATOM | 8862 | CD | ARG | B | 2163 | −14.915 | −15.079 | 34.857 | 1.00 | 196.84 | C |
| ATOM | 8863 | NE | ARG | B | 2163 | −15.694 | −14.652 | 33.705 | 1.00 | 196.72 | N |
| ATOM | 8864 | CZ | ARG | B | 2163 | −15.971 | −13.388 | 33.408 | 1.00 | 197.50 | C |
| ATOM | 8865 | NH1 | ARG | B | 2163 | −15.537 | −12.390 | 34.176 | 1.00 | 197.49 | N |
| ATOM | 8866 | NH2 | ARG | B | 2163 | −16.691 | −13.124 | 32.331 | 1.00 | 198.23 | N |
| ATOM | 8867 | N | MET | B | 2164 | −17.366 | −15.787 | 39.601 | 1.00 | 197.00 | N |
| ATOM | 8868 | CA | MET | B | 2164 | −18.036 | −16.946 | 40.238 | 1.00 | 196.84 | C |
| ATOM | 8869 | C | MET | B | 2164 | −19.522 | −16.758 | 40.518 | 1.00 | 196.85 | C |
| ATOM | 8870 | O | MET | B | 2164 | −20.005 | −15.630 | 40.659 | 1.00 | 196.91 | O |
| ATOM | 8871 | CB | MET | B | 2164 | −17.346 | −17.287 | 41.568 | 1.00 | 197.01 | C |
| ATOM | 8872 | CG | MET | B | 2164 | −16.970 | −16.048 | 42.422 | 1.00 | 197.01 | C |
| ATOM | 8873 | SD | MET | B | 2164 | −17.402 | −16.126 | 44.176 | 1.00 | 196.25 | S |
| ATOM | 8874 | CE | MET | B | 2164 | −19.151 | −15.736 | 44.103 | 1.00 | 195.76 | C |
| ATOM | 8875 | N | GLU | B | 2165 | −20.225 | −17.881 | 40.625 | 1.00 | 196.77 | N |
| ATOM | 8876 | CA | GLU | B | 2165 | −21.622 | −17.909 | 41.060 | 1.00 | 196.90 | C |
| ATOM | 8877 | C | GLU | B | 2165 | −21.706 | −18.839 | 42.279 | 1.00 | 197.41 | C |
| ATOM | 8878 | O | GLU | B | 2165 | −20.750 | −19.575 | 42.572 | 1.00 | 197.54 | O |
| ATOM | 8879 | CB | GLU | B | 2165 | −22.524 | −18.374 | 39.896 | 1.00 | 196.71 | C |
| ATOM | 8880 | CG | GLU | B | 2165 | −24.030 | −18.514 | 40.164 | 1.00 | 195.35 | C |
| ATOM | 8881 | CD | GLU | B | 2165 | −24.728 | −17.201 | 40.409 | 1.00 | 193.88 | C |
| ATOM | 8882 | OE1 | GLU | B | 2165 | −25.214 | −16.557 | 39.445 | 1.00 | 193.39 | O |
| ATOM | 8883 | OE2 | GLU | B | 2165 | −24.806 | −16.829 | 41.590 | 1.00 | 193.05 | O |
| ATOM | 8884 | N | LEU | B | 2166 | −22.825 | −18.798 | 42.999 | 1.00 | 197.78 | N |
| ATOM | 8885 | CA | LEU | B | 2166 | −23.018 | −19.672 | 44.160 | 1.00 | 197.98 | C |
| ATOM | 8886 | C | LEU | B | 2166 | −24.294 | −20.505 | 44.089 | 1.00 | 198.60 | C |
| ATOM | 8887 | O | LEU | B | 2166 | −25.368 | −20.014 | 43.714 | 1.00 | 198.63 | O |
| ATOM | 8888 | CB | LEU | B | 2166 | −22.984 | −18.860 | 45.450 | 1.00 | 197.55 | C |
| ATOM | 8889 | CG | LEU | B | 2166 | −21.642 | −18.833 | 46.159 | 1.00 | 196.23 | C |
| ATOM | 8890 | CD1 | LEU | B | 2166 | −21.395 | −17.461 | 46.690 | 1.00 | 195.43 | C |
| ATOM | 8891 | CD2 | LEU | B | 2166 | −21.644 | −19.834 | 47.273 | 1.00 | 195.44 | C |
| ATOM | 8892 | N | MET | B | 2167 | −24.159 | −21.774 | 44.458 | 1.00 | 199.26 | N |
| ATOM | 8893 | CA | MET | B | 2167 | −25.275 | −22.712 | 44.420 | 1.00 | 200.00 | C |
| ATOM | 8894 | C | MET | B | 2167 | −25.927 | −22.887 | 45.782 | 1.00 | 200.11 | C |
| ATOM | 8895 | O | MET | B | 2167 | −25.244 | −22.984 | 46.798 | 1.00 | 199.94 | O |
| ATOM | 8896 | CB | MET | B | 2167 | −24.823 | −24.083 | 43.885 | 1.00 | 200.29 | C |
| ATOM | 8897 | CG | MET | B | 2167 | −24.553 | −24.161 | 42.377 | 1.00 | 200.98 | C |
| ATOM | 8898 | SD | MET | B | 2167 | −25.910 | −23.556 | 41.350 | 1.00 | 202.25 | S |
| ATOM | 8899 | CE | MET | B | 2167 | −25.342 | −21.888 | 41.041 | 1.00 | 201.96 | C |
| ATOM | 8900 | N | GLY | B | 2168 | −27.253 | −22.934 | 45.791 | 1.00 | 200.45 | N |
| ATOM | 8901 | CA | GLY | B | 2168 | −27.983 | −23.235 | 47.004 | 1.00 | 201.05 | C |
| ATOM | 8902 | C | GLY | B | 2168 | −29.342 | −22.583 | 47.068 | 1.00 | 201.60 | C |
| ATOM | 8903 | O | GLY | B | 2168 | −29.945 | −22.277 | 46.035 | 1.00 | 201.58 | O |
| ATOM | 8904 | N | CYS | B | 2169 | −29.808 | −22.381 | 48.301 | 1.00 | 202.18 | N |
| ATOM | 8905 | CA | CYS | B | 2169 | −31.130 | −21.838 | 48.604 | 1.00 | 202.86 | C |
| ATOM | 8906 | C | CYS | B | 2169 | −31.380 | −21.822 | 50.108 | 1.00 | 203.21 | C |
| ATOM | 8907 | O | CYS | B | 2169 | −30.560 | −22.301 | 50.896 | 1.00 | 203.07 | O |
| ATOM | 8908 | CB | CYS | B | 2169 | −32.222 | −22.666 | 47.934 | 1.00 | 202.96 | C |
| ATOM | 8909 | SG | CYS | B | 2169 | −32.247 | −24.451 | 48.376 | 1.00 | 203.87 | S |
| ATOM | 8910 | N | ASP | B | 2170 | −32.536 | −21.292 | 50.497 | 1.00 | 203.74 | N |
| ATOM | 8911 | CA | ASP | B | 2170 | −32.842 | −21.095 | 51.902 | 1.00 | 204.22 | C |
| ATOM | 8912 | C | ASP | B | 2170 | −33.188 | −22.390 | 52.592 | 1.00 | 204.04 | C |
| ATOM | 8913 | O | ASP | B | 2170 | −33.616 | −23.336 | 51.943 | 1.00 | 203.94 | O |
| ATOM | 8914 | CB | ASP | B | 2170 | −33.978 | −20.098 | 52.068 | 1.00 | 204.60 | C |
| ATOM | 8915 | CG | ASP | B | 2170 | −33.943 | −19.402 | 53.425 | 1.00 | 206.47 | C |
| ATOM | 8916 | OD1 | ASP | B | 2170 | −33.765 | −18.157 | 53.437 | 1.00 | 208.30 | O |
| ATOM | 8917 | OD2 | ASP | B | 2170 | −34.078 | −20.094 | 54.474 | 1.00 | 208.18 | O |
| ATOM | 8918 | N | LEU | B | 2171 | −33.007 | −22.400 | 53.913 | 1.00 | 204.15 | N |
| ATOM | 8919 | CA | LEU | B | 2171 | −33.176 | −23.575 | 54.775 | 1.00 | 204.36 | C |
| ATOM | 8920 | C | LEU | B | 2171 | −34.253 | −24.543 | 54.275 | 1.00 | 204.93 | C |
| ATOM | 8921 | O | LEU | B | 2171 | −34.003 | −25.747 | 54.135 | 1.00 | 205.30 | O |
| ATOM | 8922 | CB | LEU | B | 2171 | −33.382 | −23.140 | 56.223 | 1.00 | 203.98 | C |
| ATOM | 8923 | CG | LEU | B | 2171 | −32.204 | −22.309 | 56.751 | 1.00 | 202.99 | C |
| ATOM | 8924 | CD1 | LEU | B | 2171 | −32.483 | −21.884 | 58.163 | 1.00 | 203.20 | C |
| ATOM | 8925 | CD2 | LEU | B | 2171 | −30.865 | −23.040 | 56.681 | 1.00 | 201.26 | C |
| ATOM | 8926 | N | ASN | B | 2172 | −35.450 | −24.017 | 54.043 | 1.00 | 205.28 | N |
| ATOM | 8927 | CA | ASN | B | 2172 | −36.243 | −24.394 | 52.863 | 1.00 | 205.48 | C |
| ATOM | 8928 | C | ASN | B | 2172 | −37.454 | −23.492 | 52.733 | 1.00 | 206.22 | C |
| ATOM | 8929 | O | ASN | B | 2172 | −38.598 | −23.877 | 53.030 | 1.00 | 206.37 | O |
| ATOM | 8930 | CB | ASN | B | 2172 | −36.550 | −25.893 | 52.694 | 1.00 | 205.00 | C |
| ATOM | 8931 | CG | ASN | B | 2172 | −36.271 | −26.384 | 51.266 | 1.00 | 203.13 | C |
| ATOM | 8932 | OD1 | ASN | B | 2172 | −36.835 | −25.882 | 50.291 | 1.00 | 200.24 | O |
| ATOM | 8933 | ND2 | ASN | B | 2172 | −35.386 | −27.364 | 51.147 | 1.00 | 201.25 | N |
| ATOM | 8934 | N | SER | B | 2173 | −37.128 | −22.254 | 52.346 | 1.00 | 206.87 | N |
| ATOM | 8935 | CA | SER | B | 2173 | −38.055 | −21.278 | 51.770 | 1.00 | 207.25 | C |
| ATOM | 8936 | C | SER | B | 2173 | −38.094 | −21.598 | 50.276 | 1.00 | 207.37 | C |
| ATOM | 8937 | O | SER | B | 2173 | −38.328 | −20.717 | 49.433 | 1.00 | 207.50 | O |
| ATOM | 8938 | CB | SER | B | 2173 | −37.568 | −19.832 | 52.023 | 1.00 | 207.30 | C |
| ATOM | 8939 | OG | SER | B | 2173 | −38.556 | −18.847 | 51.738 | 1.00 | 207.06 | O |
| ATOM | 8940 | N | CYS | B | 2174 | −37.833 | −22.875 | 49.972 | 1.00 | 207.33 | N |
| ATOM | 8941 | CA | CYS | B | 2174 | −37.997 | −23.449 | 48.640 | 1.00 | 207.17 | C |
| ATOM | 8942 | C | CYS | B | 2174 | −37.757 | −22.420 | 47.520 | 1.00 | 206.71 | C |
| ATOM | 8943 | O | CYS | B | 2174 | −38.660 | −22.078 | 46.743 | 1.00 | 206.67 | O |
| ATOM | 8944 | CB | CYS | B | 2174 | −39.365 | −24.175 | 48.524 | 1.00 | 207.46 | C |
| ATOM | 8945 | SG | CYS | B | 2174 | −40.878 | −23.258 | 49.108 | 1.00 | 207.69 | S |
| ATOM | 8946 | N | SER | B | 2175 | −36.533 | −21.905 | 47.470 | 1.00 | 205.99 | N |
| ATOM | 8947 | CA | SER | B | 2175 | −36.148 | −21.019 | 46.391 | 1.00 | 205.50 | C |
| ATOM | 8948 | C | SER | B | 2175 | −36.000 | −21.834 | 45.100 | 1.00 | 205.18 | C |
| ATOM | 8949 | O | SER | B | 2175 | −34.914 | −21.911 | 44.521 | 1.00 | 205.44 | O |
| ATOM | 8950 | CB | SER | B | 2175 | −34.862 | −20.261 | 46.742 | 1.00 | 205.50 | C |
| ATOM | 8951 | OG | SER | B | 2175 | −35.147 | −18.936 | 47.158 | 1.00 | 205.66 | O |
| ATOM | 8952 | N | MET | B | 2176 | −37.096 | −22.452 | 44.664 | 1.00 | 204.47 | N |
| ATOM | 8953 | CA | MET | B | 2176 | −37.104 | −23.236 | 43.436 | 1.00 | 203.77 | C |
| ATOM | 8954 | C | MET | B | 2176 | −38.369 | −22.896 | 42.649 | 1.00 | 203.62 | C |
| ATOM | 8955 | O | MET | B | 2176 | −39.449 | −22.856 | 43.226 | 1.00 | 203.58 | O |
| ATOM | 8956 | CB | MET | B | 2176 | −37.004 | −24.737 | 43.747 | 1.00 | 203.42 | C |
| ATOM | 8957 | CG | MET | B | 2176 | −36.250 | −25.550 | 42.685 | 1.00 | 202.93 | C |
| ATOM | 8958 | SD | MET | B | 2176 | −34.450 | −25.336 | 42.664 | 1.00 | 201.17 | S |
| ATOM | 8959 | CE | MET | B | 2176 | −34.140 | −25.152 | 40.902 | 1.00 | 201.96 | C |
| ATOM | 8960 | N | PRO | B | 2177 | −38.223 | −22.599 | 41.340 | 1.00 | 203.61 | N |
| ATOM | 8961 | CA | PRO | B | 2177 | −39.279 | −22.237 | 40.374 | 1.00 | 203.61 | C |
| ATOM | 8962 | C | PRO | B | 2177 | −40.524 | −23.108 | 40.443 | 1.00 | 203.51 | C |
| ATOM | 8963 | O | PRO | B | 2177 | −40.446 | −24.313 | 40.228 | 1.00 | 203.55 | O |
| ATOM | 8964 | CB | PRO | B | 2177 | −38.600 | −22.422 | 39.005 | 1.00 | 203.58 | C |
| ATOM | 8965 | CG | PRO | B | 2177 | −37.204 | −22.900 | 39.283 | 1.00 | 203.95 | C |
| ATOM | 8966 | CD | PRO | B | 2177 | −36.897 | −22.563 | 40.705 | 1.00 | 203.79 | C |
| ATOM | 8967 | N | LEU | B | 2178 | −41.666 | −22.481 | 40.699 | 1.00 | 203.49 | N |
| ATOM | 8968 | CA | LEU | B | 2178 | −42.885 | −23.196 | 41.052 | 1.00 | 203.51 | C |
| ATOM | 8969 | C | LEU | B | 2178 | −43.528 | −23.978 | 39.904 | 1.00 | 203.59 | C |
| ATOM | 8970 | O | LEU | B | 2178 | −44.250 | −24.949 | 40.141 | 1.00 | 203.77 | O |
| ATOM | 8971 | CB | LEU | B | 2178 | −43.888 | −22.227 | 41.664 | 1.00 | 203.48 | C |
| ATOM | 8972 | CG | LEU | B | 2178 | −44.342 | −22.564 | 43.084 | 1.00 | 203.84 | C |
| ATOM | 8973 | CD1 | LEU | B | 2178 | −44.992 | −21.348 | 43.715 | 1.00 | 204.53 | C |
| ATOM | 8974 | CD2 | LEU | B | 2178 | −45.283 | −23.767 | 43.133 | 1.00 | 204.18 | C |
| ATOM | 8975 | N | GLY | B | 2179 | −43.269 | −23.561 | 38.668 | 1.00 | 203.70 | N |
| ATOM | 8976 | CA | GLY | B | 2179 | −43.777 | −24.281 | 37.494 | 1.00 | 203.74 | C |
| ATOM | 8977 | C | GLY | B | 2179 | −44.218 | −23.423 | 36.318 | 1.00 | 203.73 | C |
| ATOM | 8978 | O | GLY | B | 2179 | −44.890 | −23.921 | 35.408 | 1.00 | 203.76 | O |
| ATOM | 8979 | N | MET | B | 2180 | −43.849 | −22.140 | 36.337 | 1.00 | 203.60 | N |
| ATOM | 8980 | CA | MET | B | 2180 | −44.157 | −21.224 | 35.244 | 1.00 | 203.43 | C |
| ATOM | 8981 | C | MET | B | 2180 | −43.246 | −21.529 | 34.073 | 1.00 | 203.35 | C |
| ATOM | 8982 | O | MET | B | 2180 | −43.674 | −21.500 | 32.922 | 1.00 | 203.26 | O |
| ATOM | 8983 | CB | MET | B | 2180 | −43.986 | −19.764 | 35.675 | 1.00 | 203.39 | C |
| ATOM | 8984 | CG | MET | B | 2180 | −45.013 | −19.265 | 36.689 | 1.00 | 203.99 | C |
| ATOM | 8985 | SD | MET | B | 2180 | −46.700 | −18.977 | 36.070 | 1.00 | 205.39 | S |
| ATOM | 8986 | CE | MET | B | 2180 | −46.633 | −17.248 | 35.621 | 1.00 | 204.81 | C |
| ATOM | 8987 | N | GLU | B | 2181 | −41.989 | −21.835 | 34.383 | 1.00 | 203.45 | N |
| ATOM | 8988 | CA | GLU | B | 2181 | −40.992 | −22.126 | 33.366 | 1.00 | 203.68 | C |
| ATOM | 8989 | C | GLU | B | 2181 | −41.185 | −23.507 | 32.804 | 1.00 | 203.57 | C |
| ATOM | 8990 | O | GLU | B | 2181 | −41.487 | −23.650 | 31.627 | 1.00 | 203.62 | O |
| ATOM | 8991 | CB | GLU | B | 2181 | −39.569 | −21.966 | 33.906 | 1.00 | 203.89 | C |
| ATOM | 8992 | CG | GLU | B | 2181 | −38.931 | −20.621 | 33.558 | 1.00 | 204.89 | C |
| ATOM | 8993 | CD | GLU | B | 2181 | −38.586 | −20.489 | 32.075 | 1.00 | 206.07 | C |
| ATOM | 8994 | OE1 | GLU | B | 2181 | −37.532 | −21.030 | 31.667 | 1.00 | 206.58 | O |
| ATOM | 8995 | OE2 | GLU | B | 2181 | −39.357 | −19.838 | 31.325 | 1.00 | 206.41 | O |
| ATOM | 8996 | N | SER | B | 2182 | −41.027 | −24.522 | 33.646 | 1.00 | 203.58 | N |
| ATOM | 8997 | CA | SER | B | 2182 | −41.219 | −25.901 | 33.210 | 1.00 | 203.75 | C |
| ATOM | 8998 | C | SER | B | 2182 | −42.623 | −26.138 | 32.634 | 1.00 | 203.65 | C |
| ATOM | 8999 | O | SER | B | 2182 | −42.857 | −27.147 | 31.967 | 1.00 | 203.75 | O |
| ATOM | 9000 | CB | SER | B | 2182 | −40.904 | −26.889 | 34.347 | 1.00 | 203.94 | C |
| ATOM | 9001 | OG | SER | B | 2182 | −41.691 | −26.649 | 35.505 | 1.00 | 204.41 | O |
| ATOM | 9002 | N | LYS | B | 2183 | −43.529 | −25.183 | 32.874 | 1.00 | 203.53 | N |
| ATOM | 9003 | CA | LYS | B | 2183 | −44.948 | −25.253 | 32.475 | 1.00 | 203.40 | C |
| ATOM | 9004 | C | LYS | B | 2183 | −45.664 | −26.411 | 33.164 | 1.00 | 203.09 | C |
| ATOM | 9005 | O | LYS | B | 2183 | −46.464 | −27.119 | 32.553 | 1.00 | 203.15 | O |
| ATOM | 9006 | CB | LYS | B | 2183 | −45.128 | −25.328 | 30.951 | 1.00 | 203.53 | C |
| ATOM | 9007 | CG | LYS | B | 2183 | −44.445 | −24.219 | 30.153 | 1.00 | 204.49 | C |
| ATOM | 9008 | CD | LYS | B | 2183 | −44.225 | −24.658 | 28.701 | 1.00 | 206.02 | C |
| ATOM | 9009 | CE | LYS | B | 2183 | −42.987 | −24.008 | 28.066 | 1.00 | 206.58 | C |
| ATOM | 9010 | NZ | LYS | B | 2183 | −42.513 | −24.792 | 26.863 | 1.00 | 206.84 | N |
| ATOM | 9011 | N | ALA | B | 2184 | −45.354 | −26.604 | 34.441 | 1.00 | 202.81 | N |
| ATOM | 9012 | CA | ALA | B | 2184 | −46.068 | −27.568 | 35.271 | 1.00 | 202.50 | C |
| ATOM | 9013 | C | ALA | B | 2184 | −47.448 | −26.991 | 35.566 | 1.00 | 202.13 | C |
| ATOM | 9014 | O | ALA | B | 2184 | −48.375 | −27.717 | 35.954 | 1.00 | 202.16 | O |
| ATOM | 9015 | CB | ALA | B | 2184 | −45.302 | −27.832 | 36.563 | 1.00 | 202.68 | C |
| ATOM | 9016 | N | ILE | B | 2185 | −47.553 | −25.675 | 35.377 | 1.00 | 201.49 | N |
| ATOM | 9017 | CA | ILE | B | 2185 | −48.810 | −24.947 | 35.445 | 1.00 | 200.89 | C |
| ATOM | 9018 | C | ILE | B | 2185 | −49.333 | −24.790 | 34.022 | 1.00 | 200.69 | C |
| ATOM | 9019 | O | ILE | B | 2185 | −48.698 | −24.140 | 33.185 | 1.00 | 200.51 | O |
| ATOM | 9020 | CB | ILE | B | 2185 | −48.620 | −23.576 | 36.115 | 1.00 | 200.78 | C |
| ATOM | 9021 | CG1 | ILE | B | 2185 | −47.955 | −23.751 | 37.486 | 1.00 | 200.31 | C |
| ATOM | 9022 | CG2 | ILE | B | 2185 | −49.956 | −22.850 | 36.228 | 1.00 | 200.65 | C |
| ATOM | 9023 | CD1 | ILE | B | 2185 | −47.320 | −22.502 | 38.042 | 1.00 | 199.32 | C |
| ATOM | 9024 | N | SER | B | 2186 | −50.485 | −25.408 | 33.759 | 1.00 | 200.52 | N |
| ATOM | 9025 | CA | SER | B | 2186 | −51.042 | −25.523 | 32.409 | 1.00 | 200.32 | C |
| ATOM | 9026 | C | SER | B | 2186 | −51.408 | −24.153 | 31.858 | 1.00 | 200.35 | C |
| ATOM | 9027 | O | SER | B | 2186 | −51.587 | −23.202 | 32.625 | 1.00 | 200.33 | O |
| ATOM | 9028 | CB | SER | B | 2186 | −52.263 | −26.453 | 32.404 | 1.00 | 200.19 | C |
| ATOM | 9029 | OG | SER | B | 2186 | −52.683 | −26.733 | 31.083 | 1.00 | 199.72 | O |
| ATOM | 9030 | N | ASP | B | 2187 | −51.497 | −24.050 | 30.531 | 1.00 | 200.39 | N |
| ATOM | 9031 | CA | ASP | B | 2187 | −51.962 | −22.817 | 29.879 | 1.00 | 200.33 | C |
| ATOM | 9032 | C | ASP | B | 2187 | −53.379 | −22.458 | 30.366 | 1.00 | 200.09 | C |
| ATOM | 9033 | O | ASP | B | 2187 | −53.708 | −21.281 | 30.546 | 1.00 | 200.10 | O |
| ATOM | 9034 | CB | ASP | B | 2187 | −51.899 | −22.932 | 28.339 | 1.00 | 200.48 | C |
| ATOM | 9035 | CG | ASP | B | 2187 | −50.611 | −22.327 | 27.735 | 1.00 | 200.59 | C |
| ATOM | 9036 | OD1 | ASP | B | 2187 | −50.571 | −22.119 | 26.501 | 1.00 | 200.52 | O |
| ATOM | 9037 | OD2 | ASP | B | 2187 | −49.642 | −22.052 | 28.479 | 1.00 | 200.62 | O |
| ATOM | 9038 | N | ALA | B | 2188 | −54.192 | −23.489 | 30.603 | 1.00 | 199.69 | N |
| ATOM | 9039 | CA | ALA | B | 2188 | −55.520 | −23.347 | 31.200 | 1.00 | 199.19 | C |
| ATOM | 9040 | C | ALA | B | 2188 | −55.492 | −22.705 | 32.601 | 1.00 | 198.77 | C |
| ATOM | 9041 | O | ALA | B | 2188 | −56.426 | −21.999 | 32.992 | 1.00 | 198.71 | O |
| ATOM | 9042 | CB | ALA | B | 2188 | −56.214 | −24.719 | 31.255 | 1.00 | 199.20 | C |
| ATOM | 9043 | N | GLN | B | 2189 | −54.405 | −22.934 | 33.332 | 1.00 | 198.23 | N |
| ATOM | 9044 | CA | GLN | B | 2189 | −54.343 | −22.644 | 34.764 | 1.00 | 197.77 | C |
| ATOM | 9045 | C | GLN | B | 2189 | −54.143 | −21.170 | 35.116 | 1.00 | 197.41 | C |
| ATOM | 9046 | O | GLN | B | 2189 | −54.122 | −20.817 | 36.295 | 1.00 | 197.46 | O |
| ATOM | 9047 | CB | GLN | B | 2189 | −53.249 | −23.498 | 35.411 | 1.00 | 197.73 | C |
| ATOM | 9048 | CG | GLN | B | 2189 | −53.597 | −24.037 | 36.787 | 1.00 | 197.83 | C |
| ATOM | 9049 | CD | GLN | B | 2189 | −52.648 | −25.131 | 37.250 | 1.00 | 197.95 | C |
| ATOM | 9050 | OE1 | GLN | B | 2189 | −52.176 | −25.938 | 36.451 | 1.00 | 198.51 | O |
| ATOM | 9051 | NE2 | GLN | B | 2189 | −52.375 | −25.170 | 38.551 | 1.00 | 198.38 | N |
| ATOM | 9052 | N | ILE | B | 2190 | −53.997 | −20.313 | 34.105 | 1.00 | 196.96 | N |
| ATOM | 9053 | CA | ILE | B | 2190 | −53.827 | −18.870 | 34.320 | 1.00 | 196.38 | C |
| ATOM | 9054 | C | ILE | B | 2190 | −54.814 | −18.073 | 33.477 | 1.00 | 196.46 | C |
| ATOM | 9055 | O | ILE | B | 2190 | −54.892 | −18.263 | 32.263 | 1.00 | 196.48 | O |
| ATOM | 9056 | CB | ILE | B | 2190 | −52.406 | −18.388 | 33.934 | 1.00 | 196.28 | C |
| ATOM | 9057 | CG1 | ILE | B | 2190 | −51.322 | −19.134 | 34.718 | 1.00 | 195.98 | C |
| ATOM | 9058 | CG2 | ILE | B | 2190 | −52.285 | −16.895 | 34.148 | 1.00 | 196.08 | C |
| ATOM | 9059 | CD1 | ILE | B | 2190 | −49.932 | −19.046 | 34.107 | 1.00 | 195.85 | C |
| ATOM | 9060 | N | THR | B | 2191 | −55.576 | −17.192 | 34.112 | 1.00 | 196.47 | N |
| ATOM | 9061 | CA | THR | B | 2191 | −56.272 | −16.157 | 33.356 | 1.00 | 196.72 | C |
| ATOM | 9062 | C | THR | B | 2191 | −55.748 | −14.800 | 33.769 | 1.00 | 197.00 | C |
| ATOM | 9063 | O | THR | B | 2191 | −54.966 | −14.680 | 34.715 | 1.00 | 196.99 | O |
| ATOM | 9064 | CB | THR | B | 2191 | −57.815 | −16.178 | 33.499 | 1.00 | 196.62 | C |
| ATOM | 9065 | OG1 | THR | B | 2191 | −58.176 | −16.598 | 34.819 | 1.00 | 196.57 | O |
| ATOM | 9066 | CG2 | THR | B | 2191 | −58.450 | −17.090 | 32.453 | 1.00 | 196.47 | C |
| ATOM | 9067 | N | ALA | B | 2192 | −56.177 | −13.784 | 33.030 | 1.00 | 197.32 | N |
| ATOM | 9068 | CA | ALA | B | 2192 | −55.817 | −12.410 | 33.307 | 1.00 | 197.45 | C |
| ATOM | 9069 | C | ALA | B | 2192 | −57.079 | −11.578 | 33.337 | 1.00 | 197.53 | C |
| ATOM | 9070 | O | ALA | B | 2192 | −58.179 | −12.086 | 33.103 | 1.00 | 197.47 | O |
| ATOM | 9071 | CB | ALA | B | 2192 | −54.874 | −11.889 | 32.244 | 1.00 | 197.48 | C |
| ATOM | 9072 | N | SER | B | 2193 | −56.897 | −10.296 | 33.627 | 1.00 | 197.69 | N |
| ATOM | 9073 | CA | SER | B | 2193 | −57.972 | −9.322 | 33.647 | 1.00 | 197.93 | C |
| ATOM | 9074 | C | SER | B | 2193 | −58.549 | −9.101 | 32.246 | 1.00 | 198.02 | C |
| ATOM | 9075 | O | SER | B | 2193 | −59.737 | −8.793 | 32.088 | 1.00 | 197.96 | O |
| ATOM | 9076 | CB | SER | B | 2193 | −57.441 | −8.018 | 34.229 | 1.00 | 197.88 | C |
| ATOM | 9077 | OG | SER | B | 2193 | −58.370 | −6.967 | 34.078 | 1.00 | 198.44 | O |
| ATOM | 9078 | N | SER | B | 2194 | −57.688 | −9.268 | 31.245 | 1.00 | 198.24 | N |
| ATOM | 9079 | CA | SER | B | 2194 | −58.049 | −9.179 | 29.835 | 1.00 | 198.54 | C |
| ATOM | 9080 | C | SER | B | 2194 | −56.798 | −9.412 | 28.997 | 1.00 | 198.75 | C |
| ATOM | 9081 | O | SER | B | 2194 | −55.680 | −9.208 | 29.474 | 1.00 | 198.74 | O |
| ATOM | 9082 | CB | SER | B | 2194 | −58.648 | −7.808 | 29.507 | 1.00 | 198.58 | C |
| ATOM | 9083 | OG | SER | B | 2194 | −57.736 | −6.764 | 29.808 | 1.00 | 198.45 | O |
| ATOM | 9084 | N | TYR | B | 2195 | −56.985 | −9.843 | 27.754 | 1.00 | 199.01 | N |
| ATOM | 9085 | CA | TYR | B | 2195 | −55.856 | −10.059 | 26.861 | 1.00 | 199.22 | C |
| ATOM | 9086 | C | TYR | B | 2195 | −56.018 | −9.352 | 25.531 | 1.00 | 199.56 | C |
| ATOM | 9087 | O | TYR | B | 2195 | −57.101 | −9.359 | 24.943 | 1.00 | 199.71 | O |
| ATOM | 9088 | CB | TYR | B | 2195 | −55.593 | −11.548 | 26.649 | 1.00 | 199.10 | C |
| ATOM | 9089 | CG | TYR | B | 2195 | −56.738 | −12.336 | 26.067 | 1.00 | 198.82 | C |
| ATOM | 9090 | CD1 | TYR | B | 2195 | −56.733 | −12.710 | 24.728 | 1.00 | 198.69 | C |
| ATOM | 9091 | CD2 | TYR | B | 2195 | −57.812 | −12.734 | 26.861 | 1.00 | 198.83 | C |
| ATOM | 9092 | CE1 | TYR | B | 2195 | −57.775 | −13.448 | 24.188 | 1.00 | 198.97 | C |
| ATOM | 9093 | CE2 | TYR | B | 2195 | −58.860 | −13.471 | 26.332 | 1.00 | 198.97 | C |
| ATOM | 9094 | CZ | TYR | B | 2195 | −58.835 | −13.826 | 24.995 | 1.00 | 199.03 | C |
| ATOM | 9095 | OH | TYR | B | 2195 | −59.870 | −14.557 | 24.463 | 1.00 | 199.03 | O |
| ATOM | 9096 | N | PHE | B | 2196 | −54.925 | −8.744 | 25.077 | 1.00 | 199.92 | N |
| ATOM | 9097 | CA | PHE | B | 2196 | −54.851 | −8.110 | 23.764 | 1.00 | 200.36 | C |
| ATOM | 9098 | C | PHE | B | 2196 | −54.903 | −9.182 | 22.682 | 1.00 | 200.83 | C |
| ATOM | 9099 | O | PHE | B | 2196 | −54.212 | −10.201 | 22.782 | 1.00 | 200.96 | O |
| ATOM | 9100 | CB | PHE | B | 2196 | −53.548 | −7.315 | 23.656 | 1.00 | 200.22 | C |
| ATOM | 9101 | CG | PHE | B | 2196 | −53.421 | −6.509 | 22.394 | 1.00 | 199.93 | C |
| ATOM | 9102 | CD1 | PHE | B | 2196 | −53.973 | −5.236 | 22.308 | 1.00 | 199.81 | C |
| ATOM | 9103 | CD2 | PHE | B | 2196 | −52.727 | −7.014 | 21.298 | 1.00 | 199.65 | C |
| ATOM | 9104 | CE1 | PHE | B | 2196 | −53.853 | −4.482 | 21.144 | 1.00 | 199.82 | C |
| ATOM | 9105 | CE2 | PHE | B | 2196 | −52.598 | −6.272 | 20.130 | 1.00 | 199.59 | C |
| ATOM | 9106 | CZ | PHE | B | 2196 | −53.162 | −5.002 | 20.052 | 1.00 | 199.81 | C |
| ATOM | 9107 | N | THR | B | 2197 | −55.722 | −8.955 | 21.655 | 1.00 | 201.39 | N |
| ATOM | 9108 | CA | THR | B | 2197 | −55.923 | −9.953 | 20.595 | 1.00 | 202.05 | C |
| ATOM | 9109 | C | THR | B | 2197 | −56.484 | −9.351 | 19.283 | 1.00 | 202.45 | C |
| ATOM | 9110 | O | THR | B | 2197 | −57.394 | −8.512 | 19.319 | 1.00 | 202.54 | O |
| ATOM | 9111 | CB | THR | B | 2197 | −56.768 | −11.179 | 21.118 | 1.00 | 202.05 | C |
| ATOM | 9112 | OG1 | THR | B | 2197 | −56.686 | −12.271 | 20.195 | 1.00 | 202.19 | O |
| ATOM | 9113 | CG2 | THR | B | 2197 | −58.242 | −10.812 | 21.383 | 1.00 | 202.19 | C |
| ATOM | 9114 | N | ASN | B | 2198 | −55.922 | −9.773 | 18.141 | 1.00 | 202.94 | N |
| ATOM | 9115 | CA | ASN | B | 2198 | −56.322 | −9.276 | 16.800 | 1.00 | 203.35 | C |
| ATOM | 9116 | C | ASN | B | 2198 | −56.307 | −10.328 | 15.660 | 1.00 | 203.60 | C |
| ATOM | 9117 | O | ASN | B | 2198 | −57.312 | −11.006 | 15.411 | 1.00 | 203.54 | O |
| ATOM | 9118 | CB | ASN | B | 2198 | −55.517 | −8.013 | 16.410 | 1.00 | 203.37 | C |
| ATOM | 9119 | CG | ASN | B | 2198 | −53.996 | −8.216 | 16.482 | 1.00 | 203.44 | C |
| ATOM | 9120 | OD1 | ASN | B | 2198 | −53.508 | −9.298 | 16.812 | 1.00 | 203.41 | O |
| ATOM | 9121 | ND2 | ASN | B | 2198 | −53.246 | −7.164 | 16.162 | 1.00 | 203.49 | N |
| ATOM | 9122 | N | MET | B | 2199 | −55.177 | −10.424 | 14.958 | 1.00 | 203.96 | N |
| ATOM | 9123 | CA | MET | B | 2199 | −54.924 | −11.467 | 13.965 | 1.00 | 204.33 | C |
| ATOM | 9124 | C | MET | B | 2199 | −53.433 | −11.819 | 13.968 | 1.00 | 204.59 | C |
| ATOM | 9125 | O | MET | B | 2199 | −53.072 | −12.995 | 14.035 | 1.00 | 204.59 | O |
| ATOM | 9126 | CB | MET | B | 2199 | −55.387 | −11.050 | 12.556 | 1.00 | 204.30 | C |
| ATOM | 9127 | CG | MET | B | 2199 | −54.464 | −10.052 | 11.830 | 1.00 | 204.37 | C |
| ATOM | 9128 | SD | MET | B | 2199 | −54.199 | −10.350 | 10.057 | 1.00 | 204.39 | S |
| ATOM | 9129 | CE | MET | B | 2199 | −53.355 | −11.937 | 10.038 | 1.00 | 204.32 | C |
| ATOM | 9130 | N | PHE | B | 2200 | −52.582 | −10.791 | 13.904 | 1.00 | 204.97 | N |
| ATOM | 9131 | CA | PHE | B | 2200 | −51.125 | −10.950 | 13.923 | 1.00 | 205.41 | C |
| ATOM | 9132 | C | PHE | B | 2200 | −50.588 | −11.144 | 15.354 | 1.00 | 205.59 | C |
| ATOM | 9133 | O | PHE | B | 2200 | −49.365 | −11.137 | 15.556 | 1.00 | 205.85 | O |
| ATOM | 9134 | CB | PHE | B | 2200 | −50.425 | −9.746 | 13.246 | 1.00 | 205.52 | C |
| ATOM | 9135 | CG | PHE | B | 2200 | −49.976 | −10.000 | 11.813 | 1.00 | 206.01 | C |
| ATOM | 9136 | CD1 | PHE | B | 2200 | −50.758 | −9.570 | 10.733 | 1.00 | 206.35 | C |
| ATOM | 9137 | CD2 | PHE | B | 2200 | −48.755 | −10.637 | 11.543 | 1.00 | 206.39 | C |
| ATOM | 9138 | CE1 | PHE | B | 2200 | −50.346 | −9.794 | 9.402 | 1.00 | 206.28 | C |
| ATOM | 9139 | CE2 | PHE | B | 2200 | −48.334 | −10.864 | 10.215 | 1.00 | 206.50 | C |
| ATOM | 9140 | CZ | PHE | B | 2200 | −49.133 | −10.440 | 9.147 | 1.00 | 206.17 | C |
| ATOM | 9141 | N | ALA | B | 2201 | −51.492 | −11.319 | 16.333 | 1.00 | 205.60 | N |
| ATOM | 9142 | CA | ALA | B | 2201 | −51.123 | −11.484 | 17.760 | 1.00 | 205.44 | C |
| ATOM | 9143 | C | ALA | B | 2201 | −52.241 | −12.042 | 18.660 | 1.00 | 205.28 | C |
| ATOM | 9144 | O | ALA | B | 2201 | −53.427 | −11.859 | 18.382 | 1.00 | 205.20 | O |
| ATOM | 9145 | CB | ALA | B | 2201 | −50.609 | −10.155 | 18.333 | 1.00 | 205.43 | C |
| ATOM | 9146 | N | THR | B | 2202 | −51.844 | −12.728 | 19.732 | 1.00 | 205.15 | N |
| ATOM | 9147 | CA | THR | B | 2202 | −52.748 | −13.109 | 20.822 | 1.00 | 205.09 | C |
| ATOM | 9148 | C | THR | B | 2202 | −51.942 | −13.148 | 22.107 | 1.00 | 205.12 | C |
| ATOM | 9149 | O | THR | B | 2202 | −51.501 | −14.214 | 22.543 | 1.00 | 205.22 | O |
| ATOM | 9150 | CB | THR | B | 2202 | −53.386 | −14.493 | 20.621 | 1.00 | 205.05 | C |
| ATOM | 9151 | OG1 | THR | B | 2202 | −53.821 | −14.632 | 19.267 | 1.00 | 205.22 | O |
| ATOM | 9152 | CG2 | THR | B | 2202 | −54.574 | −14.680 | 21.566 | 1.00 | 204.92 | C |
| ATOM | 9153 | N | TRP | B | 2203 | −51.748 | −11.983 | 22.711 | 1.00 | 205.12 | N |
| ATOM | 9154 | CA | TRP | B | 2203 | −50.916 | −11.863 | 23.904 | 1.00 | 205.08 | C |
| ATOM | 9155 | C | TRP | B | 2203 | −51.657 | −12.327 | 25.161 | 1.00 | 204.82 | C |
| ATOM | 9156 | O | TRP | B | 2203 | −51.754 | −11.599 | 26.155 | 1.00 | 204.75 | O |
| ATOM | 9157 | CB | TRP | B | 2203 | −50.421 | −10.429 | 24.060 | 1.00 | 205.40 | C |
| ATOM | 9158 | CG | TRP | B | 2203 | −49.642 | −9.921 | 22.882 | 1.00 | 205.75 | C |
| ATOM | 9159 | CD1 | TRP | B | 2203 | −50.149 | −9.359 | 21.744 | 1.00 | 206.15 | C |
| ATOM | 9160 | CD2 | TRP | B | 2203 | −48.218 | −9.911 | 22.733 | 1.00 | 206.02 | C |
| ATOM | 9161 | NE1 | TRP | B | 2203 | −49.130 | −9.001 | 20.895 | 1.00 | 206.15 | N |
| ATOM | 9162 | CE2 | TRP | B | 2203 | −47.934 | −9.328 | 21.476 | 1.00 | 206.16 | C |
| ATOM | 9163 | CE3 | TRP | B | 2203 | −47.154 | −10.338 | 23.538 | 1.00 | 206.15 | C |
| ATOM | 9164 | CZ2 | TRP | B | 2203 | −46.627 | −9.159 | 21.004 | 1.00 | 206.11 | C |
| ATOM | 9165 | CZ3 | TRP | B | 2203 | −45.857 | −10.173 | 23.071 | 1.00 | 206.21 | C |
| ATOM | 9166 | CH2 | TRP | B | 2203 | −45.605 | −9.587 | 21.812 | 1.00 | 206.15 | C |
| ATOM | 9167 | N | SER | B | 2204 | −52.157 | −13.557 | 25.092 | 1.00 | 204.47 | N |
| ATOM | 9168 | CA | SER | B | 2204 | −52.942 | −14.187 | 26.147 | 1.00 | 204.22 | C |
| ATOM | 9169 | C | SER | B | 2204 | −52.144 | −14.422 | 27.436 | 1.00 | 203.91 | C |
| ATOM | 9170 | O | SER | B | 2204 | −50.933 | −14.634 | 27.377 | 1.00 | 203.81 | O |
| ATOM | 9171 | CB | SER | B | 2204 | −53.527 | −15.505 | 25.628 | 1.00 | 204.37 | C |
| ATOM | 9172 | OG | SER | B | 2204 | −52.620 | −16.169 | 24.762 | 1.00 | 204.70 | O |
| ATOM | 9173 | N | PRO | B | 2205 | −52.827 | −14.397 | 28.606 | 1.00 | 203.68 | N |
| ATOM | 9174 | CA | PRO | B | 2205 | −52.173 | −14.526 | 29.914 | 1.00 | 203.44 | C |
| ATOM | 9175 | C | PRO | B | 2205 | −51.316 | −15.769 | 29.978 | 1.00 | 203.17 | C |
| ATOM | 9176 | O | PRO | B | 2205 | −50.277 | −15.795 | 30.642 | 1.00 | 202.98 | O |
| ATOM | 9177 | CB | PRO | B | 2205 | −53.353 | −14.666 | 30.887 | 1.00 | 203.44 | C |
| ATOM | 9178 | CG | PRO | B | 2205 | −54.549 | −14.964 | 30.039 | 1.00 | 203.51 | C |
| ATOM | 9179 | CD | PRO | B | 2205 | −54.288 | −14.261 | 28.759 | 1.00 | 203.69 | C |
| ATOM | 9180 | N | SER | B | 2206 | −51.767 | −16.778 | 29.249 | 1.00 | 202.98 | N |
| ATOM | 9181 | CA | SER | B | 2206 | −51.148 | −18.080 | 29.208 | 1.00 | 202.94 | C |
| ATOM | 9182 | C | SER | B | 2206 | −49.753 | −18.019 | 28.586 | 1.00 | 202.86 | C |
| ATOM | 9183 | O | SER | B | 2206 | −49.152 | −19.057 | 28.292 | 1.00 | 203.07 | O |
| ATOM | 9184 | CB | SER | B | 2206 | −52.052 | −19.021 | 28.413 | 1.00 | 202.97 | C |
| ATOM | 9185 | OG | SER | B | 2206 | −53.414 | −18.641 | 28.558 | 1.00 | 202.97 | O |
| ATOM | 9186 | N | LYS | B | 2207 | −49.236 | −16.808 | 28.395 | 1.00 | 202.52 | N |
| ATOM | 9187 | CA | LYS | B | 2207 | −47.918 | −16.646 | 27.805 | 1.00 | 202.31 | C |
| ATOM | 9188 | C | LYS | B | 2207 | −46.883 | −16.120 | 28.794 | 1.00 | 202.29 | C |
| ATOM | 9189 | O | LYS | B | 2207 | −45.692 | −16.264 | 28.553 | 1.00 | 202.45 | O |
| ATOM | 9190 | CB | LYS | B | 2207 | −47.978 | −15.760 | 26.553 | 1.00 | 202.23 | C |
| ATOM | 9191 | CG | LYS | B | 2207 | −48.819 | −16.318 | 25.392 | 1.00 | 202.23 | C |
| ATOM | 9192 | CD | LYS | B | 2207 | −48.035 | −17.280 | 24.493 | 1.00 | 201.92 | C |
| ATOM | 9193 | CE | LYS | B | 2207 | −48.895 | −17.837 | 23.357 | 1.00 | 201.59 | C |
| ATOM | 9194 | NZ | LYS | B | 2207 | −49.160 | −16.836 | 22.291 | 1.00 | 200.97 | N |
| ATOM | 9195 | N | ALA | B | 2208 | −47.330 | −15.536 | 29.906 | 1.00 | 202.26 | N |
| ATOM | 9196 | CA | ALA | B | 2208 | −46.431 | −14.892 | 30.883 | 1.00 | 202.24 | C |
| ATOM | 9197 | C | ALA | B | 2208 | −45.383 | −15.834 | 31.477 | 1.00 | 202.23 | C |
| ATOM | 9198 | O | ALA | B | 2208 | −45.527 | −16.311 | 32.595 | 1.00 | 202.11 | O |
| ATOM | 9199 | CB | ALA | B | 2208 | −47.244 | −14.238 | 31.991 | 1.00 | 202.24 | C |
| ATOM | 9200 | N | ARG | B | 2209 | −44.319 | −16.088 | 30.726 | 1.00 | 202.47 | N |
| ATOM | 9201 | CA | ARG | B | 2209 | −43.369 | −17.130 | 31.089 | 1.00 | 202.87 | C |
| ATOM | 9202 | C | ARG | B | 2209 | −41.930 | −16.674 | 30.971 | 1.00 | 203.44 | C |
| ATOM | 9203 | O | ARG | B | 2209 | −41.451 | −16.371 | 29.878 | 1.00 | 203.42 | O |
| ATOM | 9204 | CB | ARG | B | 2209 | −43.580 | −18.379 | 30.236 | 1.00 | 202.82 | C |
| ATOM | 9205 | CG | ARG | B | 2209 | −44.964 | −18.974 | 30.345 | 1.00 | 202.08 | C |
| ATOM | 9206 | CD | ARG | B | 2209 | −44.943 | −20.458 | 30.064 | 1.00 | 200.69 | C |
| ATOM | 9207 | NE | AEG | B | 2209 | −46.278 | −21.031 | 30.163 | 1.00 | 199.72 | N |
| ATOM | 9208 | CZ | ARG | B | 2209 | −46.977 | −21.117 | 31.289 | 1.00 | 199.40 | C |
| ATOM | 9209 | NH1 | ARG | B | 2209 | −46.476 | −20.656 | 32.428 | 1.00 | 198.94 | N |
| ATOM | 9210 | NH2 | ARG | B | 2209 | −48.187 | −21.659 | 31.274 | 1.00 | 199.56 | N |
| ATOM | 9211 | N | LEU | B | 2210 | −41.249 | −16.695 | 32.114 | 1.00 | 204.20 | N |
| ATOM | 9212 | CA | LEU | B | 2210 | −39.926 | −16.091 | 32.339 | 1.00 | 205.06 | C |
| ATOM | 9213 | C | LEU | B | 2210 | −38.968 | −15.968 | 31.146 | 1.00 | 205.32 | C |
| ATOM | 9214 | O | LEU | B | 2210 | −38.219 | −14.998 | 31.053 | 1.00 | 205.53 | O |
| ATOM | 9215 | CB | LEU | B | 2210 | −39.227 | −16.780 | 33.519 | 1.00 | 205.10 | C |
| ATOM | 9216 | CG | LEU | B | 2210 | −38.181 | −15.966 | 34.292 | 1.00 | 205.62 | C |
| ATOM | 9217 | CD1 | LEU | B | 2210 | −38.759 | −14.611 | 34.693 | 1.00 | 205.82 | C |
| ATOM | 9218 | CD2 | LEU | B | 2210 | −37.637 | −16.743 | 35.514 | 1.00 | 205.47 | C |
| ATOM | 9219 | N | HIS | B | 2211 | −38.966 | −16.956 | 30.262 | 1.00 | 205.60 | N |
| ATOM | 9220 | CA | HIS | B | 2211 | −38.327 | −16.799 | 28.970 | 1.00 | 205.98 | C |
| ATOM | 9221 | C | HIS | B | 2211 | −39.219 | −17.527 | 27.998 | 1.00 | 206.37 | C |
| ATOM | 9222 | O | HIS | B | 2211 | −39.429 | −18.733 | 28.115 | 1.00 | 206.54 | O |
| ATOM | 9223 | CB | HIS | B | 2211 | −36.913 | −17.376 | 28.965 | 1.00 | 205.96 | C |
| ATOM | 9224 | CG | HIS | B | 2211 | −36.057 | −16.887 | 30.091 | 1.00 | 205.89 | C |
| ATOM | 9225 | ND1 | HIS | B | 2211 | −36.019 | −17.508 | 31.320 | 1.00 | 205.47 | N |
| ATOM | 9226 | CD2 | HIS | B | 2211 | −35.212 | −15.832 | 30.177 | 1.00 | 206.05 | C |
| ATOM | 9227 | CE1 | HIS | B | 2211 | −35.185 | −16.863 | 32.115 | 1.00 | 205.35 | C |
| ATOM | 9228 | NE2 | HIS | B | 2211 | −34.684 | −15.841 | 31.446 | 1.00 | 205.93 | N |
| ATOM | 9229 | N | LEU | B | 2212 | −39.787 | −16.775 | 27.067 | 1.00 | 206.81 | N |
| ATOM | 9230 | CA | LEU | B | 2212 | −40.715 | −17.329 | 26.101 | 1.00 | 207.23 | C |
| ATOM | 9231 | C | LEU | B | 2212 | −40.538 | −16.603 | 24.785 | 1.00 | 207.85 | C |
| ATOM | 9232 | O | LEU | B | 2212 | −40.734 | −15.393 | 24.711 | 1.00 | 207.94 | O |
| ATOM | 9233 | CB | LEU | B | 2212 | −42.155 | −17.169 | 26.590 | 1.00 | 206.89 | C |
| ATOM | 9234 | CG | LEU | B | 2212 | −43.196 | −18.006 | 25.856 | 1.00 | 206.26 | C |
| ATOM | 9235 | CD1 | LEU | B | 2212 | −43.488 | −19.251 | 26.651 | 1.00 | 206.13 | C |
| ATOM | 9236 | CD2 | LEU | B | 2212 | −44.459 | −17.223 | 25.656 | 1.00 | 205.70 | C |
| ATOM | 9237 | N | GLN | B | 2213 | −40.156 | −17.339 | 23.748 | 1.00 | 208.67 | N |
| ATOM | 9238 | CA | GLN | B | 2213 | −40.046 | −16.747 | 22.411 | 1.00 | 209.49 | C |
| ATOM | 9239 | C | GLN | B | 2213 | −41.350 | −16.910 | 21.593 | 1.00 | 209.67 | C |
| ATOM | 9240 | O | GLN | B | 2213 | −42.234 | −17.707 | 21.954 | 1.00 | 209.82 | O |
| ATOM | 9241 | CB | GLN | B | 2213 | −38.804 | −17.275 | 21.651 | 1.00 | 209.70 | C |
| ATOM | 9242 | CG | GLN | B | 2213 | −37.450 | −17.025 | 22.347 | 1.00 | 210.30 | C |
| ATOM | 9243 | CD | GLN | B | 2213 | −37.413 | −15.712 | 23.128 | 1.00 | 211.33 | C |
| ATOM | 9244 | OE1 | GLN | B | 2213 | −37.582 | −14.624 | 22.566 | 1.00 | 211.32 | O |
| ATOM | 9245 | NE2 | GLN | B | 2213 | −37.204 | −15.817 | 24.437 | 1.00 | 211.89 | N |
| ATOM | 9246 | N | GLY | B | 2214 | −41.471 | −16.134 | 20.514 | 1.00 | 209.71 | N |
| ATOM | 9247 | CA | GLY | B | 2214 | −42.635 | −16.208 | 19.626 | 1.00 | 209.49 | C |
| ATOM | 9248 | C | GLY | B | 2214 | −42.928 | −14.899 | 18.919 | 1.00 | 209.33 | C |
| ATOM | 9249 | O | GLY | B | 2214 | −42.103 | −13.971 | 18.927 | 1.00 | 209.36 | O |
| ATOM | 9250 | N | ARG | B | 2215 | −44.098 | −14.838 | 18.283 | 1.00 | 209.10 | N |
| ATOM | 9251 | CA | ARG | B | 2215 | −44.627 | −13.577 | 17.753 | 1.00 | 208.82 | C |
| ATOM | 9252 | C | ARG | B | 2215 | −45.368 | −12.848 | 18.875 | 1.00 | 208.26 | C |
| ATOM | 9253 | O | ARG | B | 2215 | −45.130 | −11.654 | 19.103 | 1.00 | 208.49 | O |
| ATOM | 9254 | CB | ARG | B | 2215 | −45.559 | −13.799 | 16.552 | 1.00 | 208.97 | C |
| ATOM | 9255 | CG | ARG | B | 2215 | −44.868 | −14.158 | 15.240 | 1.00 | 209.17 | C |
| ATOM | 9256 | CD | ARG | B | 2215 | −45.837 | −14.034 | 14.068 | 1.00 | 209.17 | C |
| ATOM | 9257 | NE | ARG | B | 2215 | −45.707 | −12.751 | 13.381 | 1.00 | 210.04 | N |
| ATOM | 9258 | CZ | ARG | B | 2215 | −45.370 | −12.611 | 12.100 | 1.00 | 210.44 | C |
| ATOM | 9259 | NH1 | ARG | B | 2215 | −45.273 | −11.395 | 11.568 | 1.00 | 210.44 | N |
| ATOM | 9260 | NH2 | ARG | B | 2215 | −45.139 | −13.682 | 11.345 | 1.00 | 210.58 | N |
| ATOM | 9261 | N | SER | B | 2216 | −46.264 | −13.575 | 19.557 | 1.00 | 207.24 | N |
| ATOM | 9262 | CA | SER | B | 2216 | −46.913 | −13.112 | 20.789 | 1.00 | 206.05 | C |
| ATOM | 9263 | C | SER | B | 2216 | −46.333 | −13.913 | 21.941 | 1.00 | 205.18 | C |
| ATOM | 9264 | O | SER | B | 2216 | −46.814 | −14.999 | 22.265 | 1.00 | 205.01 | O |
| ATOM | 9265 | CB | SER | B | 2216 | −48.435 | −13.262 | 20.710 | 1.00 | 206.08 | C |
| ATOM | 9266 | OG | SER | B | 2216 | −48.797 | −14.468 | 20.064 | 1.00 | 206.11 | O |
| ATOM | 9267 | N | ASN | B | 2217 | −45.278 | −13.368 | 22.538 | 1.00 | 204.17 | N |
| ATOM | 9268 | CA | ASN | B | 2217 | −44.458 | −14.115 | 23.475 | 1.00 | 203.33 | C |
| ATOM | 9269 | C | ASN | B | 2217 | −44.555 | −13.646 | 24.915 | 1.00 | 202.70 | C |
| ATOM | 9270 | O | ASN | B | 2217 | −43.627 | −13.832 | 25.696 | 1.00 | 202.55 | O |
| ATOM | 9271 | CB | ASN | B | 2217 | −42.996 | −14.201 | 22.988 | 1.00 | 203.36 | C |
| ATOM | 9272 | CG | ASN | B | 2217 | −42.156 | −12.962 | 23.328 | 1.00 | 203.45 | C |
| ATOM | 9273 | OD1 | ASN | B | 2217 | −42.599 | −12.049 | 24.033 | 1.00 | 203.45 | O |
| ATOM | 9274 | ND2 | ASN | B | 2217 | −40.917 | −12.945 | 22.827 | 1.00 | 203.35 | N |
| ATOM | 9275 | N | ALA | B | 2218 | −45.686 | −13.044 | 25.262 | 1.00 | 202.09 | N |
| ATOM | 9276 | CA | ALA | B | 2218 | −45.933 | −12.622 | 26.636 | 1.00 | 201.74 | C |
| ATOM | 9277 | C | ALA | B | 2218 | −47.383 | −12.233 | 26.845 | 1.00 | 201.52 | C |
| ATOM | 9278 | O | ALA | B | 2218 | −48.149 | −12.179 | 25.887 | 1.00 | 201.68 | O |
| ATOM | 9279 | CB | ALA | B | 2218 | −45.049 | −11.475 | 26.985 | 1.00 | 201.80 | C |
| ATOM | 9280 | N | TRP | B | 2219 | −47.761 | −11.963 | 28.094 | 1.00 | 201.13 | N |
| ATOM | 9281 | CA | TRP | B | 2219 | −49.108 | −11.485 | 28.369 | 1.00 | 200.79 | C |
| ATOM | 9282 | C | TRP | B | 2219 | −49.186 | −9.984 | 28.245 | 1.00 | 200.80 | C |
| ATOM | 9283 | O | TRP | B | 2219 | −48.298 | −9.269 | 28.715 | 1.00 | 200.65 | O |
| ATOM | 9284 | CB | TRP | B | 2219 | −49.593 | −11.880 | 29.760 | 1.00 | 200.63 | C |
| ATOM | 9285 | CG | TRP | B | 2219 | −50.843 | −11.122 | 30.145 | 1.00 | 200.39 | C |
| ATOM | 9286 | CD1 | TRP | B | 2219 | −52.085 | −11.267 | 29.603 | 1.00 | 200.30 | C |
| ATOM | 9287 | CD2 | TRP | B | 2219 | −50.955 | −10.081 | 31.122 | 1.00 | 200.11 | C |
| ATOM | 9288 | NE1 | TRP | B | 2219 | −52.967 | −10.395 | 30.189 | 1.00 | 200.07 | N |
| ATOM | 9289 | CE2 | TRP | B | 2219 | −52.300 | −9.655 | 31.126 | 1.00 | 199.96 | C |
| ATOM | 9290 | CE3 | TRP | B | 2219 | −50.052 | −9.472 | 32.000 | 1.00 | 200.10 | C |
| ATOM | 9291 | CZ2 | TRP | B | 2219 | −52.765 | −8.650 | 31.972 | 1.00 | 200.17 | C |
| ATOM | 9292 | CZ3 | TRP | B | 2219 | −50.516 | −8.470 | 32.843 | 1.00 | 200.16 | C |
| ATOM | 9293 | CH2 | TRP | B | 2219 | −51.860 | −8.070 | 32.821 | 1.00 | 200.27 | C |
| ATOM | 9294 | N | ARG | B | 2220 | −50.266 | −9.524 | 27.617 | 1.00 | 200.95 | N |
| ATOM | 9295 | CA | ARG | B | 2220 | −50.619 | −8.109 | 27.582 | 1.00 | 201.16 | C |
| ATOM | 9296 | C | ARG | B | 2220 | −52.120 | −7.896 | 27.801 | 1.00 | 201.41 | C |
| ATOM | 9297 | O | ARG | B | 2220 | −52.942 | −8.639 | 27.245 | 1.00 | 201.28 | O |
| ATOM | 9298 | CB | ARG | B | 2220 | −50.186 | −7.467 | 26.266 | 1.00 | 201.09 | C |
| ATOM | 9299 | CG | ARG | B | 2220 | −48.707 | −7.140 | 26.191 | 1.00 | 200.81 | C |
| ATOM | 9300 | CD | ARG | B | 2220 | −48.321 | −6.638 | 24.813 | 1.00 | 200.02 | C |
| ATOM | 9301 | NE | ARG | B | 2220 | −49.086 | −5.449 | 24.449 | 1.00 | 199.29 | N |
| ATOM | 9302 | CZ | ARG | B | 2220 | −49.063 | −4.879 | 23.252 | 1.00 | 198.56 | C |
| ATOM | 9303 | NH1 | ARG | B | 2220 | −48.309 | −5.377 | 22.279 | 1.00 | 197.99 | N |
| ATOM | 9304 | NH2 | ARG | B | 2220 | −49.801 | −3.806 | 23.032 | 1.00 | 198.41 | N |
| ATOM | 9305 | N | PRO | B | 2221 | −52.478 | −6.871 | 28.609 | 1.00 | 201.67 | N |
| ATOM | 9306 | CA | PRO | B | 2221 | −53.873 | −6.498 | 28.880 | 1.00 | 201.86 | C |
| ATOM | 9307 | C | PRO | B | 2221 | −54.501 | −5.860 | 27.645 | 1.00 | 202.03 | C |
| ATOM | 9308 | O | PRO | B | 2221 | −53.765 | −5.355 | 26.795 | 1.00 | 202.28 | O |
| ATOM | 9309 | CB | PRO | B | 2221 | −53.738 | −5.457 | 29.996 | 1.00 | 201.84 | C |
| ATOM | 9310 | CG | PRO | B | 2221 | −52.384 | −4.861 | 29.800 | 1.00 | 201.58 | C |
| ATOM | 9311 | CD | PRO | B | 2221 | −51.526 | −5.982 | 29.309 | 1.00 | 201.60 | C |
| ATOM | 9312 | N | GLN | B | 2222 | −55.828 | −5.863 | 27.529 | 1.00 | 202.01 | N |
| ATOM | 9313 | CA | GLN | B | 2222 | −56.438 | −5.268 | 26.338 | 1.00 | 202.20 | C |
| ATOM | 9314 | C | GLN | B | 2222 | −56.098 | −3.776 | 26.201 | 1.00 | 202.18 | C |
| ATOM | 9315 | O | GLN | B | 2222 | −55.732 | −3.319 | 25.117 | 1.00 | 202.25 | O |
| ATOM | 9316 | CB | GLN | B | 2222 | −57.942 | −5.563 | 26.223 | 1.00 | 202.22 | C |
| ATOM | 9317 | CG | GLN | B | 2222 | −58.850 | −4.866 | 27.227 | 1.00 | 202.56 | C |
| ATOM | 9318 | CD | GLN | B | 2222 | −60.323 | −4.930 | 26.834 | 1.00 | 202.47 | C |
| ATOM | 9319 | OE1 | GLN | B | 2222 | −61.169 | −5.354 | 27.625 | 1.00 | 202.58 | O |
| ATOM | 9320 | NE2 | GLN | B | 2222 | −60.634 | −4.509 | 25.606 | 1.00 | 202.62 | N |
| ATOM | 9321 | N | VAL | B | 2223 | −56.188 | −3.034 | 27.301 | 1.00 | 202.22 | N |
| ATOM | 9322 | CA | VAL | B | 2223 | −55.690 | −1.654 | 27.352 | 1.00 | 202.37 | C |
| ATOM | 9323 | C | VAL | B | 2223 | −54.869 | −1.426 | 28.602 | 1.00 | 202.41 | C |
| ATOM | 9324 | O | VAL | B | 2223 | −54.647 | −2.352 | 29.379 | 1.00 | 202.41 | O |
| ATOM | 9325 | CB | VAL | B | 2223 | −56.806 | −0.592 | 27.285 | 1.00 | 202.41 | C |
| ATOM | 9326 | CG1 | VAL | B | 2223 | −56.803 | 0.105 | 25.921 | 1.00 | 202.82 | C |
| ATOM | 9327 | CG2 | VAL | B | 2223 | −58.169 | −1.192 | 27.631 | 1.00 | 202.40 | C |
| ATOM | 9328 | N | ASN | B | 2224 | −54.422 | −0.194 | 28.805 | 1.00 | 202.54 | N |
| ATOM | 9329 | CA | ASN | B | 2224 | −53.502 | 0.062 | 29.896 | 1.00 | 202.87 | C |
| ATOM | 9330 | C | ASN | B | 2224 | −54.116 | 0.783 | 31.097 | 1.00 | 203.11 | C |
| ATOM | 9331 | O | ASN | B | 2224 | −54.378 | 1.996 | 31.041 | 1.00 | 203.19 | O |
| ATOM | 9332 | CB | ASN | B | 2224 | −52.227 | 0.735 | 29.383 | 1.00 | 202.88 | C |
| ATOM | 9333 | CG | ASN | B | 2224 | −51.410 | −0.182 | 28.469 | 1.00 | 203.04 | C |
| ATOM | 9334 | OD1 | ASN | B | 2224 | −50.263 | −0.524 | 28.768 | 1.00 | 202.86 | O |
| ATOM | 9335 | ND2 | ASN | B | 2224 | −52.005 | −0.585 | 27.349 | 1.00 | 203.31 | N |
| ATOM | 9336 | N | ASN | B | 2225 | −54.348 | −0.005 | 32.160 | 1.00 | 203.26 | N |
| ATOM | 9337 | CA | ASN | B | 2225 | −54.890 | 0.431 | 33.469 | 1.00 | 203.30 | C |
| ATOM | 9338 | C | ASN | B | 2225 | −54.060 | −0.106 | 34.646 | 1.00 | 203.10 | C |
| ATOM | 9339 | O | ASN | B | 2225 | −53.593 | −1.241 | 34.593 | 1.00 | 203.24 | O |
| ATOM | 9340 | CB | ASN | B | 2225 | −56.322 | −0.082 | 33.665 | 1.00 | 203.42 | C |
| ATOM | 9341 | CG | ASN | B | 2225 | −57.318 | 0.557 | 32.722 | 1.00 | 203.97 | C |
| ATOM | 9342 | OD1 | ASN | B | 2225 | −57.485 | 1.780 | 32.696 | 1.00 | 204.39 | O |
| ATOM | 9343 | ND2 | ASN | B | 2225 | −58.013 | −0.278 | 31.957 | 1.00 | 204.78 | N |
| ATOM | 9344 | N | PRO | B | 2226 | −53.879 | 0.699 | 35.716 | 1.00 | 202.85 | N |
| ATOM | 9345 | CA | PRO | B | 2226 | −53.224 | 0.211 | 36.941 | 1.00 | 202.58 | C |
| ATOM | 9346 | C | PRO | B | 2226 | −54.066 | −0.812 | 37.706 | 1.00 | 202.27 | C |
| ATOM | 9347 | O | PRO | B | 2226 | −53.526 | −1.781 | 38.249 | 1.00 | 202.08 | O |
| ATOM | 9348 | CB | PRO | B | 2226 | −53.040 | 1.484 | 37.775 | 1.00 | 202.68 | C |
| ATOM | 9349 | CG | PRO | B | 2226 | −53.208 | 2.627 | 36.800 | 1.00 | 202.87 | C |
| ATOM | 9350 | CD | PRO | B | 2226 | −54.222 | 2.128 | 35.825 | 1.00 | 202.90 | C |
| ATOM | 9351 | N | LYS | B | 2227 | −55.379 | −0.598 | 37.736 | 1.00 | 202.02 | N |
| ATOM | 9352 | CA | LYS | B | 2227 | −56.300 | −1.571 | 38.306 | 1.00 | 201.86 | C |
| ATOM | 9353 | C | LYS | B | 2227 | −56.432 | −2.816 | 37.410 | 1.00 | 201.87 | C |
| ATOM | 9354 | O | LYS | B | 2227 | −57.397 | −3.571 | 37.503 | 1.00 | 201.82 | O |
| ATOM | 9355 | CB | LYS | B | 2227 | −57.659 | −0.922 | 38.594 | 1.00 | 201.83 | C |
| ATOM | 9356 | CG | LYS | B | 2227 | −57.731 | −0.231 | 39.951 | 1.00 | 201.64 | C |
| ATOM | 9357 | CD | LYS | B | 2227 | −59.149 | 0.209 | 40.290 | 1.00 | 201.58 | C |
| ATOM | 9358 | CE | LYS | B | 2227 | −59.223 | 0.843 | 41.677 | 1.00 | 201.20 | C |
| ATOM | 9359 | NZ | LYS | B | 2227 | −60.481 | 1.625 | 41.898 | 1.00 | 200.82 | N |
| ATOM | 9360 | N | GLU | B | 2228 | −55.440 | −3.024 | 36.551 | 1.00 | 201.83 | N |
| ATOM | 9361 | CA | GLU | B | 2228 | −55.352 | −4.218 | 35.723 | 1.00 | 201.86 | C |
| ATOM | 9362 | C | GLU | B | 2228 | −54.510 | −5.277 | 36.406 | 1.00 | 201.64 | C |
| ATOM | 9363 | O | GLU | B | 2228 | −53.621 | −4.942 | 37.191 | 1.00 | 201.66 | O |
| ATOM | 9364 | CB | GLU | B | 2228 | −54.728 | −3.871 | 34.383 | 1.00 | 202.00 | C |
| ATOM | 9365 | CG | GLU | B | 2228 | −55.730 | −3.702 | 33.275 | 1.00 | 203.14 | C |
| ATOM | 9366 | CD | GLU | B | 2228 | −56.250 | −5.035 | 32.777 | 1.00 | 204.95 | C |
| ATOM | 9367 | OE1 | GLU | B | 2228 | −55.705 | −6.084 | 33.201 | 1.00 | 205.35 | O |
| ATOM | 9368 | OE2 | GLU | B | 2228 | −57.204 | −5.034 | 31.962 | 1.00 | 205.84 | O |
| ATOM | 9369 | N | TRP | B | 2229 | −54.774 | −6.548 | 36.102 | 1.00 | 201.39 | N |
| ATOM | 9370 | CA | TRP | B | 2229 | −54.100 | −7.637 | 36.810 | 1.00 | 201.26 | C |
| ATOM | 9371 | C | TRP | B | 2229 | −53.866 | −8.910 | 36.016 | 1.00 | 200.94 | C |
| ATOM | 9372 | O | TRP | B | 2229 | −54.428 | −9.096 | 34.938 | 1.00 | 200.83 | O |
| ATOM | 9373 | CB | TRP | B | 2229 | −54.846 | −7.986 | 38.103 | 1.00 | 201.64 | C |
| ATOM | 9374 | CG | TRP | B | 2229 | −56.326 | −8.286 | 37.940 | 1.00 | 202.32 | C |
| ATOM | 9375 | CD1 | TRP | B | 2229 | −57.370 | −7.463 | 38.273 | 1.00 | 202.57 | C |
| ATOM | 9376 | CD2 | TRP | B | 2229 | −56.921 | −9.498 | 37.434 | 1.00 | 202.89 | C |
| ATOM | 9377 | NE1 | TRP | B | 2229 | −58.570 | −8.079 | 37.999 | 1.00 | 202.64 | N |
| ATOM | 9378 | CE2 | TRP | B | 2229 | −58.325 | −9.325 | 37.483 | 1.00 | 202.69 | C |
| ATOM | 9379 | CE3 | TRP | B | 2229 | −56.404 | −10.707 | 36.938 | 1.00 | 202.89 | C |
| ATOM | 9380 | CZ2 | TRP | B | 2229 | −59.218 | −10.315 | 37.053 | 1.00 | 202.25 | C |
| ATOM | 9381 | CZ3 | TRP | B | 2229 | −57.294 | −11.691 | 36.517 | 1.00 | 202.36 | C |
| ATOM | 9382 | CH2 | TRP | B | 2229 | −58.685 | −11.486 | 36.576 | 1.00 | 202.32 | C |
| ATOM | 9383 | N | LEU | B | 2230 | −53.032 | −9.781 | 36.586 | 1.00 | 200.69 | N |
| ATOM | 9384 | CA | LEU | B | 2230 | −52.801 | −11.138 | 36.086 | 1.00 | 200.58 | C |
| ATOM | 9385 | C | LEU | B | 2230 | −52.904 | −12.161 | 37.219 | 1.00 | 200.36 | C |
| ATOM | 9386 | O | LEU | B | 2230 | −52.361 | −11.946 | 38.302 | 1.00 | 200.33 | O |
| ATOM | 9387 | CB | LEU | B | 2230 | −51.425 | −11.241 | 35.427 | 1.00 | 200.63 | C |
| ATOM | 9388 | CG | LEU | B | 2230 | −50.929 | −12.647 | 35.065 | 1.00 | 200.81 | C |
| ATOM | 9389 | CD1 | LEU | B | 2230 | −51.776 | −13.260 | 33.959 | 1.00 | 201.21 | C |
| ATOM | 9390 | CD2 | LEU | B | 2230 | −49.463 | −12.623 | 34.663 | 1.00 | 200.72 | C |
| ATOM | 9391 | N | GLN | B | 2231 | −53.578 | −13.280 | 36.957 | 1.00 | 200.16 | N |
| ATOM | 9392 | CA | GLN | B | 2231 | −53.891 | −14.256 | 38.010 | 1.00 | 199.89 | C |
| ATOM | 9393 | C | GLN | B | 2231 | −53.405 | −15.671 | 37.700 | 1.00 | 199.77 | C |
| ATOM | 9394 | O | GLN | B | 2231 | −53.808 | −16.291 | 36.705 | 1.00 | 199.77 | O |
| ATOM | 9395 | CB | GLN | B | 2231 | −55.408 | −14.259 | 38.313 | 1.00 | 200.05 | C |
| ATOM | 9396 | CG | GLN | B | 2231 | −55.871 | −15.159 | 39.478 | 1.00 | 199.48 | C |
| ATOM | 9397 | CD | GLN | B | 2231 | −57.391 | −15.191 | 39.662 | 1.00 | 199.45 | C |
| ATOM | 9398 | OE1 | GLN | B | 2231 | −57.890 | −15.753 | 40.631 | 1.00 | 199.17 | O |
| ATOM | 9399 | NE2 | GLN | B | 2231 | −58.124 | −14.595 | 38.733 | 1.00 | 198.99 | N |
| ATOM | 9400 | N | VAL | B | 2232 | −52.534 | −16.170 | 38.570 | 1.00 | 199.41 | N |
| ATOM | 9401 | CA | VAL | B | 2232 | −52.204 | −17.589 | 38.600 | 1.00 | 199.27 | C |
| ATOM | 9402 | C | VAL | B | 2232 | −53.213 | −18.299 | 39.502 | 1.00 | 198.90 | C |
| ATOM | 9403 | O | VAL | B | 2232 | −53.649 | −17.749 | 40.509 | 1.00 | 199.04 | O |
| ATOM | 9404 | CB | VAL | B | 2232 | −50.732 | −17.841 | 39.080 | 1.00 | 199.40 | C |
| ATOM | 9405 | CG1 | VAL | B | 2232 | −50.550 | −19.238 | 39.698 | 1.00 | 199.56 | C |
| ATOM | 9406 | CG2 | VAL | B | 2232 | −49.742 | −17.640 | 37.930 | 1.00 | 199.59 | C |
| ATOM | 9407 | N | ASP | B | 2233 | −53.599 | −19.507 | 39.114 | 1.00 | 198.38 | N |
| ATOM | 9408 | CA | ASP | B | 2233 | −54.405 | −20.369 | 39.954 | 1.00 | 197.93 | C |
| ATOM | 9409 | C | ASP | B | 2233 | −53.559 | −21.611 | 40.167 | 1.00 | 197.58 | C |
| ATOM | 9410 | O | ASP | B | 2233 | −53.180 | −22.269 | 39.204 | 1.00 | 197.60 | O |
| ATOM | 9411 | CB | ASP | B | 2233 | −55.736 | −20.686 | 39.239 | 1.00 | 198.05 | C |
| ATOM | 9412 | CG | ASP | B | 2233 | −56.587 | −21.752 | 39.948 | 1.00 | 198.04 | C |
| ATOM | 9413 | OD1 | ASP | B | 2233 | −57.444 | −22.348 | 39.256 | 1.00 | 197.76 | O |
| ATOM | 9414 | OD2 | ASP | B | 2233 | −56.424 | −21.995 | 41.166 | 1.00 | 198.15 | O |
| ATOM | 9415 | N | PHE | B | 2234 | −53.206 | −21.896 | 41.416 | 1.00 | 197.13 | N |
| ATOM | 9416 | CA | PHE | B | 2234 | −52.647 | −23.201 | 41.744 | 1.00 | 196.73 | C |
| ATOM | 9417 | C | PHE | B | 2234 | −53.827 | −24.118 | 42.021 | 1.00 | 196.68 | C |
| ATOM | 9418 | O | PHE | B | 2234 | −54.755 | −23.763 | 42.752 | 1.00 | 196.50 | O |
| ATOM | 9419 | CB | PHE | B | 2234 | −51.746 | −23.150 | 42.973 | 1.00 | 196.50 | C |
| ATOM | 9420 | CG | PHE | B | 2234 | −50.700 | −22.076 | 42.935 | 1.00 | 196.02 | C |
| ATOM | 9421 | CD1 | PHE | B | 2234 | −51.028 | −20.750 | 43.173 | 1.00 | 196.09 | C |
| ATOM | 9422 | CD2 | PHE | B | 2234 | −49.376 | −22.397 | 42.711 | 1.00 | 195.66 | C |
| ATOM | 9423 | CE1 | PHE | B | 2234 | −50.052 | −19.758 | 43.161 | 1.00 | 196.21 | C |
| ATOM | 9424 | CE2 | PHE | B | 2234 | −48.394 | −21.413 | 42.704 | 1.00 | 195.68 | C |
| ATOM | 9425 | CZ | PHE | B | 2234 | −48.732 | −20.093 | 42.922 | 1.00 | 195.93 | C |
| ATOM | 9426 | N | GLN | B | 2235 | −53.807 | −25.298 | 41.430 | 1.00 | 196.69 | N |
| ATOM | 9427 | CA | GLN | B | 2235 | −54.884 | −26.233 | 41.674 | 1.00 | 196.92 | C |
| ATOM | 9428 | C | GLN | B | 2235 | −54.752 | −26.844 | 43.085 | 1.00 | 196.95 | C |
| ATOM | 9429 | O | GLN | B | 2235 | −55.501 | −27.765 | 43.442 | 1.00 | 197.23 | O |
| ATOM | 9430 | CB | GLN | B | 2235 | −54.944 | −27.300 | 40.571 | 1.00 | 196.98 | C |
| ATOM | 9431 | CG | GLN | B | 2235 | −55.271 | −26.755 | 39.165 | 1.00 | 197.15 | C |
| ATOM | 9432 | CD | GLN | B | 2235 | −55.268 | −27.837 | 38.076 | 1.00 | 197.23 | C |
| ATOM | 9433 | OE1 | GLN | B | 2235 | −54.402 | −27.848 | 37.194 | 1.00 | 197.24 | O |
| ATOM | 9434 | NE2 | GLN | B | 2235 | −56.240 | −28.750 | 38.138 | 1.00 | 197.72 | N |
| ATOM | 9435 | N | LYS | B | 2236 | −53.801 | −26.324 | 43.876 | 1.00 | 196.78 | N |
| ATOM | 9436 | CA | LYS | B | 2236 | −53.645 | −26.681 | 45.309 | 1.00 | 196.46 | C |
| ATOM | 9437 | C | LYS | B | 2236 | −53.035 | −25.548 | 46.152 | 1.00 | 196.36 | C |
| ATOM | 9438 | O | LYS | B | 2236 | −52.680 | −24.495 | 45.617 | 1.00 | 196.35 | O |
| ATOM | 9439 | CB | LYS | B | 2236 | −52.863 | −27.996 | 45.500 | 1.00 | 196.48 | C |
| ATOM | 9440 | CG | LYS | B | 2236 | −51.402 | −27.968 | 45.070 | 1.00 | 196.19 | C |
| ATOM | 9441 | CD | LYS | B | 2236 | −50.683 | −29.213 | 45.547 | 1.00 | 196.14 | C |
| ATOM | 9442 | CE | LYS | B | 2236 | −49.475 | −29.520 | 44.684 | 1.00 | 195.41 | C |
| ATOM | 9443 | NZ | LYS | B | 2236 | −48.795 | −30.757 | 45.149 | 1.00 | 195.00 | N |
| ATOM | 9444 | N | THR | B | 2237 | −52.917 | −25.765 | 47.464 | 1.00 | 196.05 | N |
| ATOM | 9445 | CA | THR | B | 2237 | −52.445 | −24.718 | 48.385 | 1.00 | 195.71 | C |
| ATOM | 9446 | C | THR | B | 2237 | −50.924 | −24.633 | 48.481 | 1.00 | 195.21 | C |
| ATOM | 9447 | O | THR | B | 2237 | −50.263 | −25.605 | 48.863 | 1.00 | 195.08 | O |
| ATOM | 9448 | CB | THR | B | 2237 | −53.077 | −24.863 | 49.787 | 1.00 | 195.83 | C |
| ATOM | 9449 | OG1 | THR | B | 2237 | −54.406 | −24.326 | 49.757 | 1.00 | 195.99 | O |
| ATOM | 9450 | CG2 | THR | B | 2237 | −52.262 | −24.112 | 50.850 | 1.00 | 196.04 | C |
| ATOM | 9451 | N | MET | B | 2238 | −50.400 | −23.449 | 48.158 | 1.00 | 194.64 | N |
| ATOM | 9452 | CA | MET | B | 2238 | −48.959 | −23.230 | 47.997 | 1.00 | 194.24 | C |
| ATOM | 9453 | C | MET | B | 2238 | −48.352 | −22.220 | 48.977 | 1.00 | 193.37 | C |
| ATOM | 9454 | O | MET | B | 2238 | −49.062 | −21.436 | 49.605 | 1.00 | 193.42 | O |
| ATOM | 9455 | CB | MET | B | 2238 | −48.639 | −22.799 | 46.557 | 1.00 | 194.74 | C |
| ATOM | 9456 | CG | MET | B | 2238 | −49.066 | −23.797 | 45.468 | 1.00 | 196.07 | C |
| ATOM | 9457 | SD | MET | B | 2238 | −48.166 | −25.380 | 45.321 | 1.00 | 198.93 | 5 |
| ATOM | 9458 | CE | MET | B | 2238 | −48.691 | −25.950 | 43.686 | 1.00 | 197.32 | C |
| ATOM | 9459 | N | LYS | B | 2239 | −47.025 | −22.272 | 49.093 | 1.00 | 192.21 | N |
| ATOM | 9460 | CA | LYS | B | 2239 | −46.227 | −21.330 | 49.866 | 1.00 | 191.08 | C |
| ATOM | 9461 | C | LYS | B | 2239 | −45.355 | −20.585 | 48.855 | 1.00 | 190.84 | C |
| ATOM | 9462 | O | LYS | B | 2239 | −44.839 | −21.205 | 47.935 | 1.00 | 190.98 | O |
| ATOM | 9463 | CB | LYS | B | 2239 | −45.351 | −22.097 | 50.861 | 1.00 | 190.71 | C |
| ATOM | 9464 | CG | LYS | B | 2239 | −44.823 | −21.266 | 52.019 | 1.00 | 189.32 | C |
| ATOM | 9465 | CD | LYS | B | 2239 | −43.746 | −22.003 | 52.797 | 1.00 | 186.57 | C |
| ATOM | 9466 | CE | LYS | B | 2239 | −43.099 | −21.088 | 53.818 | 1.00 | 184.89 | C |
| ATOM | 9467 | NZ | LYS | B | 2239 | −41.791 | −21.632 | 54.253 | 1.00 | 183.80 | N |
| ATOM | 9468 | N | VAL | B | 2240 | −45.182 | −19.272 | 49.012 | 1.00 | 190.40 | N |
| ATOM | 9469 | CA | VAL | B | 2240 | −44.462 | −18.457 | 48.011 | 1.00 | 189.95 | C |
| ATOM | 9470 | C | VAL | B | 2240 | −43.255 | −17.699 | 48.588 | 1.00 | 189.91 | C |
| ATOM | 9471 | O | VAL | B | 2240 | −43.369 | −17.058 | 49.626 | 1.00 | 189.91 | O |
| ATOM | 9472 | CB | VAL | B | 2240 | −45.426 | −17.436 | 47.338 | 1.00 | 189.84 | C |
| ATOM | 9473 | CG1 | VAL | B | 2240 | −44.696 | −16.573 | 46.327 | 1.00 | 189.51 | C |
| ATOM | 9474 | CG2 | VAL | B | 2240 | −46.597 | −18.150 | 46.681 | 1.00 | 189.55 | C |
| ATOM | 9475 | N | THR | B | 2241 | −42.106 | −17.769 | 47.918 | 1.00 | 189.92 | N |
| ATOM | 9476 | CA | THR | B | 2241 | −40.964 | −16.902 | 48.258 | 1.00 | 190.11 | C |
| ATOM | 9477 | C | THR | B | 2241 | −41.103 | −15.537 | 47.591 | 1.00 | 190.26 | C |
| ATOM | 9478 | O | THR | B | 2241 | −41.053 | −14.500 | 48.260 | 1.00 | 190.17 | O |
| ATOM | 9479 | CB | THR | B | 2241 | −39.589 | −17.549 | 47.898 | 1.00 | 190.21 | C |
| ATOM | 9480 | OG1 | THR | B | 2241 | −39.257 | −18.516 | 48.898 | 1.00 | 190.25 | O |
| ATOM | 9481 | CG2 | THR | B | 2241 | −38.436 | −16.498 | 47.792 | 1.00 | 189.68 | C |
| ATOM | 9482 | N | GLY | B | 2242 | −41.280 | −15.550 | 46.271 | 1.00 | 190.37 | N |
| ATOM | 9483 | CA | GLY | B | 2242 | −41.391 | −14.324 | 45.500 | 1.00 | 190.40 | C |
| ATOM | 9484 | C | GLY | B | 2242 | −41.566 | −14.566 | 44.020 | 1.00 | 190.43 | C |
| ATOM | 9485 | O | GLY | B | 2242 | −41.292 | −15.663 | 43.526 | 1.00 | 190.21 | O |
| ATOM | 9486 | N | VAL | B | 2243 | −42.018 | −13.520 | 43.328 | 1.00 | 190.71 | N |
| ATOM | 9487 | CA | VAL | B | 2243 | −42.311 | −13.558 | 41.893 | 1.00 | 191.24 | C |
| ATOM | 9488 | C | VAL | B | 2243 | −41.196 | −12.924 | 41.059 | 1.00 | 191.79 | C |
| ATOM | 9489 | O | VAL | B | 2243 | −40.800 | −11.779 | 41.320 | 1.00 | 191.97 | O |
| ATOM | 9490 | CB | VAL | B | 2243 | −43.636 | −12.837 | 41.556 | 1.00 | 191.10 | C |
| ATOM | 9491 | CG1 | VAL | B | 2243 | −44.842 | −13.702 | 41.914 | 1.00 | 191.09 | C |
| ATOM | 9492 | CG2 | VAL | B | 2243 | −43.702 | −11.487 | 42.248 | 1.00 | 190.95 | C |
| ATOM | 9493 | N | THR | B | 2244 | −40.705 | −13.674 | 40.062 | 1.00 | 192.31 | N |
| ATOM | 9494 | CA | THR | B | 2244 | −39.618 | −13.240 | 39.166 | 1.00 | 192.63 | C |
| ATOM | 9495 | C | THR | B | 2244 | −40.232 | −12.600 | 37.933 | 1.00 | 192.95 | C |
| ATOM | 9496 | O | THR | B | 2244 | −40.903 | −13.280 | 37.151 | 1.00 | 193.04 | O |
| ATOM | 9497 | CB | THR | B | 2244 | −38.705 | −14.428 | 38.736 | 1.00 | 192.49 | C |
| ATOM | 9498 | OG1 | THR | B | 2244 | −37.936 | −14.890 | 39.850 | 1.00 | 192.44 | O |
| ATOM | 9499 | CG2 | THR | B | 2244 | −37.741 | −13.993 | 37.686 | 1.00 | 192.57 | C |
| ATOM | 9500 | N | THR | B | 2245 | −40.018 | −11.295 | 37.771 | 1.00 | 193.29 | N |
| ATOM | 9501 | CA | THR | B | 2245 | −40.672 | −10.562 | 36.689 | 1.00 | 193.81 | C |
| ATOM | 9502 | C | THR | B | 2245 | −39.702 | −10.178 | 35.594 | 1.00 | 194.29 | C |
| ATOM | 9503 | O | THR | B | 2245 | −38.485 | −10.148 | 35.804 | 1.00 | 194.22 | O |
| ATOM | 9504 | CB | THR | B | 2245 | −41.456 | −9.311 | 37.167 | 1.00 | 193.76 | C |
| ATOM | 9505 | OG1 | THR | B | 2245 | −40.710 | −8.615 | 38.172 | 1.00 | 194.09 | O |
| ATOM | 9506 | CG2 | THR | B | 2245 | −42.829 | −9.698 | 37.708 | 1.00 | 193.41 | C |
| ATOM | 9507 | N | GLN | B | 2246 | −40.280 | −9.866 | 34.435 | 1.00 | 194.97 | N |
| ATOM | 9508 | CA | GLN | B | 2246 | −39.557 | −9.640 | 33.197 | 1.00 | 195.54 | C |
| ATOM | 9509 | C | GLN | B | 2246 | −40.504 | −9.015 | 32.161 | 1.00 | 196.00 | C |
| ATOM | 9510 | O | GLN | B | 2246 | −41.730 | −9.117 | 32.274 | 1.00 | 195.87 | O |
| ATOM | 9511 | CB | GLN | B | 2246 | −39.022 | −10.977 | 32.700 | 1.00 | 195.48 | C |
| ATOM | 9512 | CG | GLN | B | 2246 | −37.955 | −10.896 | 31.657 | 1.00 | 195.68 | C |
| ATOM | 9513 | CD | GLN | B | 2246 | −38.166 | −11.941 | 30.594 | 1.00 | 196.17 | C |
| ATOM | 9514 | OE1 | GLN | B | 2246 | −39.293 | −12.372 | 30.356 | 1.00 | 195.77 | O |
| ATOM | 9515 | NE2 | GLN | B | 2246 | −37.085 | −12.359 | 29.945 | 1.00 | 197.14 | N |
| ATOM | 9516 | N | GLY | B | 2247 | −39.927 | −8.356 | 31.161 | 1.00 | 196.72 | N |
| ATOM | 9517 | CA | GLY | B | 2247 | −40.697 | −7.772 | 30.059 | 1.00 | 197.51 | C |
| ATOM | 9518 | C | GLY | B | 2247 | −40.574 | −8.508 | 28.730 | 1.00 | 197.97 | C |
| ATOM | 9519 | O | GLY | B | 2247 | −40.037 | −9.617 | 28.666 | 1.00 | 198.18 | O |
| ATOM | 9520 | N | VAL | B | 2248 | −41.077 | −7.901 | 27.660 | 1.00 | 198.26 | N |
| ATOM | 9521 | CA | VAL | B | 2248 | −41.067 | −8.560 | 26.366 | 1.00 | 198.59 | C |
| ATOM | 9522 | C | VAL | B | 2248 | −40.279 | −7.783 | 25.347 | 1.00 | 199.12 | C |
| ATOM | 9523 | O | VAL | B | 2248 | −40.240 | −6.543 | 25.362 | 1.00 | 199.12 | O |
| ATOM | 9524 | CB | VAL | B | 2248 | −42.478 | −8.816 | 25.814 | 1.00 | 198.42 | C |
| ATOM | 9525 | CG1 | VAL | B | 2248 | −43.357 | −9.330 | 26.894 | 1.00 | 198.57 | C |
| ATOM | 9526 | CG2 | VAL | B | 2248 | −43.080 | −7.552 | 25.239 | 1.00 | 198.38 | C |
| ATOM | 9527 | N | LYS | B | 2249 | −39.653 | −8.548 | 24.463 | 1.00 | 199.81 | N |
| ATOM | 9528 | CA | LYS | B | 2249 | −38.931 | −8.032 | 23.302 | 1.00 | 200.34 | C |
| ATOM | 9529 | C | LYS | B | 2249 | −39.867 | −8.139 | 22.082 | 1.00 | 200.37 | C |
| ATOM | 9530 | O | LYS | B | 2249 | −39.931 | −9.192 | 21.423 | 1.00 | 200.75 | O |
| ATOM | 9531 | CB | LYS | B | 2249 | −37.620 | −8.835 | 23.101 | 1.00 | 200.59 | C |
| ATOM | 9532 | CG | LYS | B | 2249 | −37.631 | −10.303 | 23.654 | 1.00 | 200.51 | C |
| ATOM | 9533 | CD | LYS | B | 2249 | −36.842 | −11.273 | 22.775 | 1.00 | 200.36 | C |
| ATOM | 9534 | CE | LYS | B | 2249 | −37.623 | −11.621 | 21.503 | 1.00 | 200.43 | C |
| ATOM | 9535 | NZ | LYS | B | 2249 | −36.797 | −11.504 | 20.258 | 1.00 | 200.36 | N |
| ATOM | 9536 | N | SER | B | 2250 | −40.608 | −7.065 | 21.790 | 1.00 | 200.00 | N |
| ATOM | 9537 | CA | SER | B | 2250 | −41.703 | −7.156 | 20.806 | 1.00 | 199.57 | C |
| ATOM | 9538 | C | SER | B | 2250 | −41.292 | −6.934 | 19.346 | 1.00 | 199.54 | C |
| ATOM | 9539 | O | SER | B | 2250 | −42.167 | −6.745 | 18.485 | 1.00 | 199.62 | O |
| ATOM | 9540 | CB | SER | B | 2250 | −42.854 | −6.220 | 21.168 | 1.00 | 199.43 | C |
| ATOM | 9541 | OG | SER | B | 2250 | −43.972 | −6.464 | 20.333 | 1.00 | 198.74 | O |
| ATOM | 9542 | N | LEU | B | 2251 | −39.977 | −6.974 | 19.080 | 1.00 | 199.21 | N |
| ATOM | 9543 | CA | LEU | B | 2251 | −39.364 | −6.603 | 17.774 | 1.00 | 198.56 | C |
| ATOM | 9544 | C | LEU | B | 2251 | −39.794 | −5.181 | 17.290 | 1.00 | 198.23 | C |
| ATOM | 9545 | O | LEU | B | 2251 | −39.048 | −4.489 | 16.576 | 1.00 | 198.30 | O |
| ATOM | 9546 | CB | LEU | B | 2251 | −39.533 | −7.714 | 16.699 | 1.00 | 198.41 | C |
| ATOM | 9547 | CG | LEU | B | 2251 | −38.890 | −9.116 | 16.865 | 1.00 | 197.57 | C |
| ATOM | 9548 | CD1 | LEU | B | 2251 | −37.538 | −9.075 | 17.592 | 1.00 | 196.67 | C |
| ATOM | 9549 | CD2 | LEU | B | 2251 | −39.817 | −10.138 | 17.535 | 1.00 | 196.33 | C |
| ATOM | 9550 | N | LEU | B | 2252 | −40.994 | −4.770 | 17.712 | 1.00 | 197.45 | N |
| ATOM | 9551 | CA | LEU | B | 2252 | −41.473 | −3.394 | 17.649 | 1.00 | 196.77 | C |
| ATOM | 9552 | C | LEU | B | 2252 | −40.558 | −2.518 | 18.525 | 1.00 | 196.34 | C |
| ATOM | 9553 | O | LEU | B | 2252 | −39.536 | −1.995 | 18.037 | 1.00 | 196.04 | O |
| ATOM | 9554 | CB | LEU | B | 2252 | −42.944 | −3.360 | 18.121 | 1.00 | 196.80 | C |
| ATOM | 9555 | CG | LEU | B | 2252 | −43.862 | −2.123 | 18.128 | 1.00 | 197.09 | C |
| ATOM | 9556 | CD1 | LEU | B | 2252 | −44.182 | −1.590 | 16.719 | 1.00 | 196.32 | C |
| ATOM | 9557 | CD2 | LEU | B | 2252 | −45.157 | −2.406 | 18.918 | 1.00 | 196.53 | C |
| ATOM | 9558 | N | THR | B | 2253 | −40.922 | −2.397 | 19.809 | 1.00 | 195.89 | N |
| ATOM | 9559 | CA | THR | B | 2253 | −40.164 | −1.657 | 20.830 | 1.00 | 195.58 | C |
| ATOM | 9560 | C | THR | B | 2253 | −39.970 | −2.514 | 22.074 | 1.00 | 195.50 | C |
| ATOM | 9561 | O | THR | B | 2253 | −40.418 | −3.665 | 22.130 | 1.00 | 195.62 | O |
| ATOM | 9562 | CB | THR | B | 2253 | −40.923 | −0.416 | 21.330 | 1.00 | 195.53 | C |
| ATOM | 9563 | OG1 | THR | B | 2253 | −42.290 | −0.771 | 21.565 | 1.00 | 195.11 | O |
| ATOM | 9564 | CG2 | THR | B | 2253 | −40.826 | 0.773 | 20.346 | 1.00 | 195.77 | C |
| ATOM | 9565 | N | SER | B | 2254 | −39.313 | −1.925 | 23.075 | 1.00 | 195.26 | N |
| ATOM | 9566 | CA | SER | B | 2254 | −39.213 | −2.501 | 24.417 | 1.00 | 195.03 | C |
| ATOM | 9567 | C | SER | B | 2254 | −40.581 | −2.510 | 25.099 | 1.00 | 194.74 | C |
| ATOM | 9568 | O | SER | B | 2254 | −41.419 | −1.670 | 24.791 | 1.00 | 194.80 | O |
| ATOM | 9569 | CB | SER | B | 2254 | −38.231 | −1.688 | 25.269 | 1.00 | 195.00 | C |
| ATOM | 9570 | OG | SER | B | 2254 | −36.902 | −2.165 | 25.144 | 1.00 | 195.23 | O |
| ATOM | 9571 | N | MET | B | 2255 | −40.801 | −3.460 | 26.010 | 1.00 | 194.37 | N |
| ATOM | 9572 | CA | MET | B | 2255 | −41.992 | −3.478 | 26.877 | 1.00 | 194.00 | C |
| ATOM | 9573 | C | MET | B | 2255 | −41.738 | −4.232 | 28.174 | 1.00 | 194.00 | C |
| ATOM | 9574 | O | MET | B | 2255 | −41.300 | −5.381 | 28.129 | 1.00 | 194.18 | O |
| ATOM | 9575 | CB | MET | B | 2255 | −43.159 | −4.122 | 26.162 | 1.00 | 193.88 | C |
| ATOM | 9576 | CG | MET | B | 2255 | −44.235 | −3.166 | 25.774 | 1.00 | 193.75 | C |
| ATOM | 9577 | SD | MET | B | 2255 | −45.301 | −3.935 | 24.552 | 1.00 | 193.81 | S |
| ATOM | 9578 | CE | MET | B | 2255 | −44.291 | −3.789 | 23.077 | 1.00 | 194.50 | C |
| ATOM | 9579 | N | TYR | B | 2256 | −42.012 | −3.586 | 29.314 | 1.00 | 193.78 | N |
| ATOM | 9580 | CA | TYR | B | 2256 | −41.781 | −4.161 | 30.661 | 1.00 | 193.66 | C |
| ATOM | 9581 | C | TYR | B | 2256 | −42.553 | −3.407 | 31.745 | 1.00 | 193.63 | C |
| ATOM | 9582 | O | TYR | B | 2256 | −43.038 | −2.303 | 31.490 | 1.00 | 193.81 | O |
| ATOM | 9583 | CB | TYR | B | 2256 | −40.282 | −4.213 | 31.023 | 1.00 | 193.58 | C |
| ATOM | 9584 | CG | TYR | B | 2256 | −39.545 | −2.885 | 30.979 | 1.00 | 193.43 | C |
| ATOM | 9585 | CD1 | TYR | B | 2256 | −39.605 | −1.987 | 32.041 | 1.00 | 193.22 | C |
| ATOM | 9586 | CD2 | TYR | B | 2256 | −38.765 | −2.541 | 29.881 | 1.00 | 193.71 | C |
| ATOM | 9587 | CE1 | TYR | B | 2256 | −38.921 | −0.771 | 31.999 | 1.00 | 193.41 | C |
| ATOM | 9588 | CE2 | TYR | B | 2256 | −38.074 | −1.328 | 29.827 | 1.00 | 193.70 | C |
| ATOM | 9589 | CZ | TYR | B | 2256 | −38.153 | −0.449 | 30.889 | 1.00 | 193.59 | C |
| ATOM | 9590 | OH | TYR | B | 2256 | −37.472 | 0.753 | 30.830 | 1.00 | 193.80 | O |
| ATOM | 9591 | N | VAL | B | 2257 | −42.658 | −3.990 | 32.945 | 1.00 | 193.40 | N |
| ATOM | 9592 | CA | VAL | B | 2257 | −43.367 | −3.340 | 34.056 | 1.00 | 193.13 | C |
| ATOM | 9593 | C | VAL | B | 2257 | −42.414 | −2.891 | 35.160 | 1.00 | 193.58 | C |
| ATOM | 9594 | O | VAL | B | 2257 | −41.648 | −3.696 | 35.697 | 1.00 | 193.51 | O |
| ATOM | 9595 | CB | VAL | B | 2257 | −44.456 | −4.225 | 34.648 | 1.00 | 192.76 | C |
| ATOM | 9596 | CG1 | VAL | B | 2257 | −45.305 | −3.422 | 35.580 | 1.00 | 192.34 | C |
| ATOM | 9597 | CG2 | VAL | B | 2257 | −45.318 | −4.792 | 33.557 | 1.00 | 192.53 | C |
| ATOM | 9598 | N | LYS | B | 2258 | −42.495 | −1.601 | 35.493 | 1.00 | 194.12 | N |
| ATOM | 9599 | CA | LYS | B | 2258 | −41.541 | −0.910 | 36.374 | 1.00 | 194.65 | C |
| ATOM | 9600 | C | LYS | B | 2258 | −41.806 | −1.102 | 37.874 | 1.00 | 194.88 | C |
| ATOM | 9601 | O | LYS | B | 2258 | −40.861 | −1.286 | 38.642 | 1.00 | 194.93 | O |
| ATOM | 9602 | CB | LYS | B | 2258 | −41.510 | 0.588 | 36.030 | 1.00 | 194.66 | C |
| ATOM | 9603 | CG | LYS | B | 2258 | −40.444 | 1.415 | 36.748 | 1.00 | 195.52 | C |
| ATOM | 9604 | CD | LYS | B | 2258 | −39.037 | 0.886 | 36.476 | 1.00 | 196.92 | C |
| ATOM | 9605 | CE | LYS | B | 2258 | −37.986 | 1.997 | 36.525 | 1.00 | 197.61 | C |
| ATOM | 9606 | NZ | LYS | B | 2258 | −37.565 | 2.330 | 37.913 | 1.00 | 197.90 | N |
| ATOM | 9607 | N | GLU | B | 2259 | −43.082 | −1.029 | 38.272 | 1.00 | 195.15 | N |
| ATOM | 9608 | CA | GLU | B | 2259 | −43.532 | −1.226 | 39.663 | 1.00 | 195.23 | C |
| ATOM | 9609 | C | GLU | B | 2259 | −44.840 | −1.991 | 39.719 | 1.00 | 195.34 | C |
| ATOM | 9610 | O | GLU | B | 2259 | −45.714 | −1.785 | 38.882 | 1.00 | 195.46 | O |
| ATOM | 9611 | CB | GLU | B | 2259 | −43.784 | 0.107 | 40.345 | 1.00 | 195.08 | C |
| ATOM | 9612 | CG | GLU | B | 2259 | −42.565 | 0.823 | 40.799 | 1.00 | 195.57 | C |
| ATOM | 9613 | CD | GLU | B | 2259 | −42.818 | 2.299 | 40.892 | 1.00 | 196.70 | C |
| ATOM | 9614 | OE1 | GLU | B | 2259 | −43.581 | 2.716 | 41.792 | 1.00 | 197.22 | O |
| ATOM | 9615 | OE2 | GLU | B | 2259 | −42.264 | 3.044 | 40.052 | 1.00 | 197.52 | O |
| ATOM | 9616 | N | PHE | B | 2260 | −45.002 | −2.837 | 40.730 | 1.00 | 195.55 | N |
| ATOM | 9617 | CA | PHE | B | 2260 | −46.262 | −3.562 | 40.892 | 1.00 | 195.79 | C |
| ATOM | 9618 | C | PHE | B | 2260 | −46.676 | −3.894 | 42.337 | 1.00 | 196.08 | C |
| ATOM | 9619 | O | PHE | B | 2260 | −45.928 | −3.662 | 43.295 | 1.00 | 196.01 | O |
| ATOM | 9620 | CB | PHE | B | 2260 | −46.265 | −4.824 | 40.024 | 1.00 | 195.65 | C |
| ATOM | 9621 | CG | PHE | B | 2260 | −45.308 | −5.884 | 40.481 | 1.00 | 195.68 | C |
| ATOM | 9622 | CD1 | PHE | B | 2260 | −45.782 | −7.082 | 40.991 | 1.00 | 195.46 | C |
| ATOM | 9623 | CD2 | PHE | B | 2260 | −43.930 | −5.695 | 40.387 | 1.00 | 195.90 | C |
| ATOM | 9624 | CE1 | PHE | B | 2260 | −44.899 | −8.073 | 41.404 | 1.00 | 195.60 | C |
| ATOM | 9625 | CE2 | PHE | B | 2260 | −43.040 | −6.684 | 40.801 | 1.00 | 195.79 | C |
| ATOM | 9626 | CZ | PHE | B | 2260 | −43.525 | −7.872 | 41.310 | 1.00 | 195.47 | C |
| ATOM | 9627 | N | LEU | B | 2261 | −47.896 | −4.414 | 42.461 | 1.00 | 196.41 | N |
| ATOM | 9628 | CA | LEU | B | 2261 | −48.459 | −4.878 | 43.719 | 1.00 | 196.73 | C |
| ATOM | 9629 | C | LEU | B | 2261 | −48.789 | −6.352 | 43.579 | 1.00 | 197.29 | C |
| ATOM | 9630 | O | LEU | B | 2261 | −48.794 | −6.886 | 42.466 | 1.00 | 197.30 | O |
| ATOM | 9631 | CB | LEU | B | 2261 | −49.746 | −4.125 | 44.031 | 1.00 | 196.43 | C |
| ATOM | 9632 | CG | LEU | B | 2261 | −49.729 | −2.617 | 43.848 | 1.00 | 195.96 | C |
| ATOM | 9633 | CD1 | LEU | B | 2261 | −51.123 | −2.059 | 44.003 | 1.00 | 195.60 | C |
| ATOM | 9634 | CD2 | LEU | B | 2261 | −48.771 | −1.979 | 44.835 | 1.00 | 195.91 | C |
| ATOM | 9635 | N | ILE | B | 2262 | −49.061 | −7.009 | 44.705 | 1.00 | 198.00 | N |
| ATOM | 9636 | CA | ILE | B | 2262 | −49.578 | −8.379 | 44.673 | 1.00 | 198.66 | C |
| ATOM | 9637 | C | ILE | B | 2262 | −50.794 | −8.552 | 45.580 | 1.00 | 199.37 | C |
| ATOM | 9638 | O | ILE | B | 2262 | −50.945 | −7.840 | 46.577 | 1.00 | 199.38 | O |
| ATOM | 9639 | CB | ILE | B | 2262 | −48.516 | −9.435 | 45.055 | 1.00 | 198.48 | C |
| ATOM | 9640 | CG1 | ILE | B | 2262 | −47.110 | −8.852 | 45.015 | 1.00 | 198.00 | C |
| ATOM | 9641 | CG2 | ILE | B | 2262 | −48.623 | −10.654 | 44.140 | 1.00 | 198.36 | C |
| ATOM | 9642 | CD1 | ILE | B | 2262 | −46.040 | −9.870 | 45.266 | 1.00 | 197.46 | C |
| ATOM | 9643 | N | SER | B | 2263 | −51.655 | −9.501 | 45.215 | 1.00 | 200.32 | N |
| ATOM | 9644 | CA | SER | B | 2263 | −52.790 | −9.891 | 46.053 | 1.00 | 201.48 | C |
| ATOM | 9645 | C | SER | B | 2263 | −52.896 | −11.417 | 46.265 | 1.00 | 202.09 | C |
| ATOM | 9646 | O | SER | B | 2263 | −52.542 | −12.209 | 45.381 | 1.00 | 202.28 | O |
| ATOM | 9647 | CB | SER | B | 2263 | −54.107 | −9.298 | 45.515 | 1.00 | 201.56 | C |
| ATOM | 9648 | OG | SER | B | 2263 | −54.449 | −9.787 | 44.226 | 1.00 | 202.02 | O |
| ATOM | 9649 | N | SER | B | 2264 | −53.374 | −11.818 | 47.444 | 1.00 | 202.73 | N |
| ATOM | 9650 | CA | SER | B | 2264 | −53.528 | −13.232 | 47.781 | 1.00 | 203.30 | C |
| ATOM | 9651 | C | SER | B | 2264 | −54.980 | −13.602 | 48.074 | 1.00 | 203.68 | C |
| ATOM | 9652 | O | SER | B | 2264 | −55.792 | −12.746 | 48.453 | 1.00 | 203.89 | O |
| ATOM | 9653 | CB | SER | B | 2264 | −52.651 | −13.593 | 48.981 | 1.00 | 203.34 | C |
| ATOM | 9654 | OG | SER | B | 2264 | −52.918 | −12.751 | 50.094 | 1.00 | 203.67 | O |
| ATOM | 9655 | N | SER | B | 2265 | −55.293 | −14.884 | 47.902 | 1.00 | 204.01 | N |
| ATOM | 9656 | CA | SER | B | 2265 | −56.619 | −15.411 | 48.213 | 1.00 | 204.28 | C |
| ATOM | 9657 | C | SER | B | 2265 | −56.577 | −16.894 | 48.589 | 1.00 | 204.43 | C |
| ATOM | 9658 | O | SER | B | 2265 | −55.662 | −17.624 | 48.195 | 1.00 | 204.55 | O |
| ATOM | 9659 | CB | SER | B | 2265 | −57.563 | −15.202 | 47.028 | 1.00 | 204.21 | C |
| ATOM | 9660 | OG | SER | B | 2265 | −58.895 | −15.531 | 47.368 | 1.00 | 204.40 | O |
| ATOM | 9661 | N | GLN | B | 2266 | −57.569 | −17.325 | 49.363 | 1.00 | 204.51 | N |
| ATOM | 9662 | CA | GLN | B | 2266 | −57.776 | −18.742 | 49.634 | 1.00 | 204.49 | C |
| ATOM | 9663 | C | GLN | B | 2266 | −59.003 | −19.219 | 48.885 | 1.00 | 204.63 | C |
| ATOM | 9664 | O | GLN | B | 2266 | −59.031 | −20.341 | 48.375 | 1.00 | 204.64 | O |
| ATOM | 9665 | CB | GLN | B | 2266 | −57.920 | −19.005 | 51.134 | 1.00 | 204.41 | C |
| ATOM | 9666 | CG | GLN | B | 2266 | −56.673 | −18.681 | 51.947 | 1.00 | 204.16 | C |
| ATOM | 9667 | CD | GLN | B | 2266 | −55.377 | −18.922 | 51.179 | 1.00 | 203.81 | C |
| ATOM | 9668 | OE1 | GLN | B | 2266 | −55.131 | −20.018 | 50.659 | 1.00 | 203.20 | O |
| ATOM | 9669 | NE2 | GLN | B | 2266 | −54.542 | −17.888 | 51.104 | 1.00 | 203.78 | N |
| ATOM | 9670 | N | ASP | B | 2267 | −60.005 | −18.344 | 48.816 | 1.00 | 204.84 | N |
| ATOM | 9671 | CA | ASP | B | 2267 | −61.257 | −18.609 | 48.103 | 1.00 | 205.11 | C |
| ATOM | 9672 | C | ASP | B | 2267 | −61.130 | −18.500 | 46.574 | 1.00 | 205.18 | C |
| ATOM | 9673 | O | ASP | B | 2267 | −61.759 | −19.264 | 45.835 | 1.00 | 205.24 | O |
| ATOM | 9674 | CB | ASP | B | 2267 | −62.405 | −17.725 | 48.639 | 1.00 | 205.15 | C |
| ATOM | 9675 | CG | ASP | B | 2267 | −62.025 | −16.244 | 48.760 | 1.00 | 205.23 | C |
| ATOM | 9676 | OD1 | ASP | B | 2267 | −62.885 | −15.386 | 48.464 | 1.00 | 205.07 | O |
| ATOM | 9677 | OD2 | ASP | B | 2267 | −60.885 | −15.932 | 49.165 | 1.00 | 205.55 | O |
| ATOM | 9678 | N | GLY | B | 2268 | −60.318 | −17.554 | 46.107 | 1.00 | 205.26 | N |
| ATOM | 9679 | CA | GLY | B | 2268 | −60.093 | −17.365 | 44.672 | 1.00 | 205.35 | C |
| ATOM | 9680 | C | GLY | B | 2268 | −61.096 | −16.453 | 43.990 | 1.00 | 205.38 | C |
| ATOM | 9681 | O | GLY | B | 2268 | −61.135 | −16.380 | 42.753 | 1.00 | 205.41 | O |
| ATOM | 9682 | N | HIS | B | 2269 | −61.901 | −15.764 | 44.806 | 1.00 | 205.32 | N |
| ATOM | 9683 | CA | HIS | B | 2269 | −62.953 | −14.853 | 44.332 | 1.00 | 205.19 | C |
| ATOM | 9684 | C | HIS | B | 2269 | −62.987 | −13.537 | 45.133 | 1.00 | 204.75 | C |
| ATOM | 9685 | O | HIS | B | 2269 | −63.585 | −12.555 | 44.692 | 1.00 | 204.77 | O |
| ATOM | 9686 | CB | HIS | B | 2269 | −64.331 | −15.545 | 44.348 | 1.00 | 205.40 | C |
| ATOM | 9687 | CG | HIS | B | 2269 | −64.307 | −16.977 | 43.886 | 1.00 | 206.32 | C |
| ATOM | 9688 | ND1 | HIS | B | 2269 | −64.590 | −18.037 | 44.725 | 1.00 | 206.98 | N |
| ATOM | 9689 | CD2 | HIS | B | 2269 | −64.027 | −17.523 | 42.677 | 1.00 | 206.98 | C |
| ATOM | 9690 | CE1 | HIS | B | 2269 | −64.489 | −19.171 | 44.053 | 1.00 | 206.95 | C |
| ATOM | 9691 | NE2 | HIS | B | 2269 | −64.145 | −18.887 | 42.809 | 1.00 | 207.16 | N |
| ATOM | 9692 | N | GLN | B | 2270 | −62.344 | −13.530 | 46.302 | 1.00 | 204.23 | N |
| ATOM | 9693 | CA | GLN | B | 2270 | −62.176 | −12.319 | 47.116 | 1.00 | 203.80 | C |
| ATOM | 9694 | C | GLN | B | 2270 | −60.751 | −12.239 | 47.673 | 1.00 | 203.34 | C |
| ATOM | 9695 | O | GLN | B | 2270 | −60.174 | −13.259 | 48.047 | 1.00 | 203.42 | O |
| ATOM | 9696 | CB | GLN | B | 2270 | −63.209 | −12.271 | 48.238 | 1.00 | 203.92 | C |
| ATOM | 9697 | CG | GLN | B | 2270 | −63.412 | −10.883 | 48.805 | 1.00 | 204.37 | C |
| ATOM | 9698 | CD | GLN | B | 2270 | −64.866 | −10.467 | 48.803 | 1.00 | 204.78 | C |
| ATOM | 9699 | OE1 | GLN | B | 2270 | −65.729 | −11.173 | 49.330 | 1.00 | 204.81 | O |
| ATOM | 9700 | NE2 | GLN | B | 2270 | −65.149 | −9.311 | 48.205 | 1.00 | 204.84 | N |
| ATOM | 9701 | N | TRP | B | 2271 | −60.194 | −11.032 | 47.749 | 1.00 | 202.67 | N |
| ATOM | 9702 | CA | TRP | B | 2271 | −58.735 | −10.889 | 47.773 | 1.00 | 202.14 | C |
| ATOM | 9703 | C | TRP | B | 2271 | −58.127 | −10.047 | 48.900 | 1.00 | 201.93 | C |
| ATOM | 9704 | O | TRP | B | 2271 | −58.381 | −8.851 | 48.995 | 1.00 | 201.86 | O |
| ATOM | 9705 | CB | TRP | B | 2271 | −58.271 | −10.363 | 46.407 | 1.00 | 201.97 | C |
| ATOM | 9706 | CG | TRP | B | 2271 | −58.845 | −11.151 | 45.262 | 1.00 | 201.61 | C |
| ATOM | 9707 | CD1 | TRP | B | 2271 | −60.068 | −10.978 | 44.667 | 1.00 | 201.39 | C |
| ATOM | 9708 | CD2 | TRP | B | 2271 | −58.233 | −12.254 | 44.600 | 1.00 | 201.11 | C |
| ATOM | 9709 | NE1 | TRP | B | 2271 | −60.247 | −11.906 | 43.671 | 1.00 | 201.02 | N |
| ATOM | 9710 | CE2 | TRP | B | 2271 | −59.134 | −12.700 | 43.608 | 1.00 | 201.10 | C |
| ATOM | 9711 | CE3 | TRP | B | 2271 | −57.004 | −12.906 | 44.744 | 1.00 | 200.79 | C |
| ATOM | 9712 | CZ2 | TRP | B | 2271 | −58.844 | −13.769 | 42.768 | 1.00 | 201.27 | C |
| ATOM | 9713 | CZ3 | TRP | B | 2271 | −56.717 | −13.962 | 43.913 | 1.00 | 201.18 | C |
| ATOM | 9714 | CH2 | TRP | B | 2271 | −57.632 | −14.386 | 42.937 | 1.00 | 201.41 | C |
| ATOM | 9715 | N | THR | B | 2272 | −57.311 | −10.678 | 49.739 | 0.50 | 201.82 | N |
| ATOM | 9716 | CA | THR | B | 2272 | −56.538 | −9.958 | 50.746 | 0.50 | 201.78 | C |
| ATOM | 9717 | C | THR | B | 2272 | −55.158 | −9.699 | 50.160 | 0.50 | 201.79 | C |
| ATOM | 9718 | O | THR | B | 2272 | −54.384 | −10.631 | 49.965 | 0.50 | 201.67 | O |
| ATOM | 9719 | CB | THR | B | 2272 | −56.414 | −10.760 | 52.063 | 0.50 | 201.80 | C |
| ATOM | 9720 | OG1 | THR | B | 2272 | −57.692 | −11.295 | 52.428 | 0.50 | 201.85 | O |
| ATOM | 9721 | CG2 | THR | B | 2272 | −55.900 | −9.876 | 53.196 | 0.50 | 201.70 | C |
| ATOM | 9722 | N | LEU | B | 2273 | −54.853 | −8.438 | 49.871 | 1.00 | 201.95 | N |
| ATOM | 9723 | CA | LEU | B | 2273 | −53.607 | −8.106 | 49.153 | 1.00 | 202.29 | C |
| ATOM | 9724 | C | LEU | B | 2273 | −52.357 | −8.026 | 50.052 | 1.00 | 202.64 | C |
| ATOM | 9725 | O | LEU | B | 2273 | −52.455 | −8.200 | 51.267 | 1.00 | 202.67 | O |
| ATOM | 9726 | CB | LEU | B | 2273 | −53.786 | −6.872 | 48.234 | 1.00 | 202.17 | C |
| ATOM | 9727 | CG | LEU | B | 2273 | −53.412 | −5.419 | 48.546 | 1.00 | 201.45 | C |
| ATOM | 9728 | CD1 | LEU | B | 2273 | −52.079 | −5.068 | 47.921 | 1.00 | 200.36 | C |
| ATOM | 9729 | CD2 | LEU | B | 2273 | −54.482 | −4.511 | 47.990 | 1.00 | 200.62 | C |
| ATOM | 9730 | N | PHE | B | 2274 | −51.193 | −7.783 | 49.441 | 1.00 | 203.05 | N |
| ATOM | 9731 | CA | PHE | B | 2274 | −49.906 | −7.789 | 50.146 | 1.00 | 203.50 | C |
| ATOM | 9732 | C | PHE | B | 2274 | −49.551 | −6.451 | 50.790 | 1.00 | 204.00 | C |
| ATOM | 9733 | O | PHE | B | 2274 | −49.823 | −5.385 | 50.222 | 1.00 | 204.22 | O |
| ATOM | 9734 | CB | PHE | B | 2274 | −48.770 | −8.211 | 49.207 | 1.00 | 203.30 | C |
| ATOM | 9735 | CG | PHE | B | 2274 | −47.519 | −8.656 | 49.927 | 1.00 | 203.14 | C |
| ATOM | 9736 | CD1 | PHE | B | 2274 | −46.500 | −7.754 | 50.211 | 1.00 | 203.20 | C |
| ATOM | 9737 | CD2 | PHE | B | 2274 | −47.361 | −9.981 | 50.323 | 1.00 | 202.75 | C |
| ATOM | 9738 | CE1 | PHE | B | 2274 | −45.345 | −8.169 | 50.882 | 1.00 | 203.11 | C |
| ATOM | 9739 | CE2 | PHE | B | 2274 | −46.206 | −10.400 | 50.988 | 1.00 | 202.40 | C |
| ATOM | 9740 | CZ | PHE | B | 2274 | −45.200 | −9.493 | 51.265 | 1.00 | 202.54 | C |
| ATOM | 9741 | N | PHE | B | 2275 | −48.914 | −6.531 | 51.962 | 1.00 | 204.48 | N |
| ATOM | 9742 | CA | PHE | B | 2275 | −48.517 | −5.359 | 52.751 | 1.00 | 204.77 | C |
| ATOM | 9743 | C | PHE | B | 2275 | −47.077 | −5.387 | 53.268 | 1.00 | 204.70 | C |
| ATOM | 9744 | O | PHE | B | 2275 | −46.458 | −6.445 | 53.408 | 1.00 | 204.49 | O |
| ATOM | 9745 | CB | PHE | B | 2275 | −49.467 | −5.169 | 53.934 | 1.00 | 205.09 | C |
| ATOM | 9746 | CG | PHE | B | 2275 | −50.714 | −4.405 | 53.596 | 1.00 | 205.78 | C |
| ATOM | 9747 | CD1 | PHE | B | 2275 | −51.818 | −5.051 | 53.041 | 1.00 | 206.42 | C |
| ATOM | 9748 | CD2 | PHE | B | 2275 | −50.790 | −3.036 | 53.841 | 1.00 | 206.55 | C |
| ATOM | 9749 | CE1 | PHE | B | 2275 | −52.982 | −4.340 | 52.729 | 1.00 | 206.83 | C |
| ATOM | 9750 | CE2 | PHE | B | 2275 | −51.950 | −2.310 | 53.537 | 1.00 | 206.95 | C |
| ATOM | 9751 | CZ | PHE | B | 2275 | −53.048 | −2.964 | 52.980 | 1.00 | 206.63 | C |
| ATOM | 9752 | N | GLN | B | 2276 | −46.569 | −4.195 | 53.559 | 1.00 | 204.79 | N |
| ATOM | 9753 | CA | GLN | B | 2276 | −45.251 | −4.013 | 54.141 | 1.00 | 204.98 | C |
| ATOM | 9754 | C | GLN | B | 2276 | −45.309 | −4.244 | 55.639 | 1.00 | 204.79 | C |
| ATOM | 9755 | O | GLN | B | 2276 | −45.803 | −5.269 | 56.115 | 1.00 | 204.92 | O |
| ATOM | 9756 | CB | GLN | B | 2276 | −44.772 | −2.576 | 53.892 | 1.00 | 205.17 | C |
| ATOM | 9757 | CG | GLN | B | 2276 | −44.023 | −2.354 | 52.589 | 1.00 | 206.22 | C |
| ATOM | 9758 | CD | GLN | B | 2276 | −42.557 | −2.762 | 52.664 | 1.00 | 207.22 | C |
| ATOM | 9759 | OE1 | GLN | B | 2276 | −42.159 | −3.569 | 53.510 | 1.00 | 207.55 | O |
| ATOM | 9760 | NE2 | GLN | B | 2276 | −41.747 | −2.208 | 51.765 | 1.00 | 207.61 | N |
| ATOM | 9761 | N | ASN | B | 2277 | −44.771 | −3.264 | 56.359 | 1.00 | 204.50 | N |
| ATOM | 9762 | CA | ASN | B | 2277 | −44.916 | −3.101 | 57.800 | 1.00 | 204.12 | C |
| ATOM | 9763 | C | ASN | B | 2277 | −45.854 | −1.917 | 58.041 | 1.00 | 203.67 | C |
| ATOM | 9764 | O | ASN | B | 2277 | −46.213 | −1.601 | 59.180 | 1.00 | 203.57 | O |
| ATOM | 9765 | CB | ASN | B | 2277 | −43.542 | −2.846 | 58.429 | 1.00 | 204.26 | C |
| ATOM | 9766 | CG | ASN | B | 2277 | −42.515 | −2.305 | 57.419 | 1.00 | 204.51 | C |
| ATOM | 9767 | OD1 | ASN | B | 2277 | −42.849 | −1.535 | 56.508 | 1.00 | 204.52 | O |
| ATOM | 9768 | ND2 | ASN | B | 2277 | −41.260 | −2.718 | 57.581 | 1.00 | 204.71 | N |
| ATOM | 9769 | N | GLY | B | 2278 | −46.230 | −1.274 | 56.933 | 1.00 | 203.15 | N |
| ATOM | 9770 | CA | GLY | B | 2278 | −47.219 | −0.199 | 56.898 | 1.00 | 202.51 | C |
| ATOM | 9771 | C | GLY | B | 2278 | −48.188 | −0.417 | 55.744 | 1.00 | 201.98 | C |
| ATOM | 9772 | O | GLY | B | 2278 | −48.912 | −1.414 | 55.729 | 1.00 | 202.07 | O |
| ATOM | 9773 | N | LYS | B | 2279 | −48.193 | 0.501 | 54.771 | 1.00 | 201.29 | N |
| ATOM | 9774 | CA | LYS | B | 2279 | −49.095 | 0.418 | 53.609 | 1.00 | 200.38 | C |
| ATOM | 9775 | C | LYS | B | 2279 | −48.656 | −0.677 | 52.632 | 1.00 | 199.83 | C |
| ATOM | 9776 | O | LYS | B | 2279 | −47.785 | −1.490 | 52.947 | 1.00 | 199.59 | O |
| ATOM | 9777 | CB | LYS | B | 2279 | −49.225 | 1.773 | 52.883 | 1.00 | 200.32 | C |
| ATOM | 9778 | CG | LYS | B | 2279 | −49.522 | 3.010 | 53.751 | 1.00 | 199.96 | C |
| ATOM | 9779 | CD | LYS | B | 2279 | −50.852 | 2.947 | 54.498 | 1.00 | 199.61 | C |
| ATOM | 9780 | CE | LYS | B | 2279 | −50.635 | 2.666 | 55.980 | 1.00 | 199.56 | C |
| ATOM | 9781 | NZ | LYS | B | 2279 | −51.868 | 2.840 | 56.790 | 1.00 | 199.52 | N |
| ATOM | 9782 | N | VAL | B | 2280 | −49.273 | −0.694 | 51.453 | 1.00 | 199.24 | N |
| ATOM | 9783 | CA | VAL | B | 2280 | −49.032 | −1.735 | 50.454 | 1.00 | 198.75 | C |
| ATOM | 9784 | C | VAL | B | 2280 | −47.591 | −1.749 | 49.980 | 1.00 | 198.58 | C |
| ATOM | 9785 | O | VAL | B | 2280 | −46.871 | −0.757 | 50.128 | 1.00 | 198.48 | O |
| ATOM | 9786 | CB | VAL | B | 2280 | −49.952 | −1.587 | 49.231 | 1.00 | 198.67 | C |
| ATOM | 9787 | CG1 | VAL | B | 2280 | −51.340 | −2.095 | 49.551 | 1.00 | 198.52 | C |
| ATOM | 9788 | CG2 | VAL | B | 2280 | −49.991 | −0.140 | 48.758 | 1.00 | 198.59 | C |
| ATOM | 9789 | N | LYS | B | 2281 | −47.179 | −2.879 | 49.409 | 1.00 | 198.36 | N |
| ATOM | 9790 | CA | LYS | B | 2281 | −45.812 | −3.029 | 48.952 | 1.00 | 198.21 | C |
| ATOM | 9791 | C | LYS | B | 2281 | −45.628 | −2.534 | 47.522 | 1.00 | 198.61 | C |
| ATOM | 9792 | O | LYS | B | 2281 | −46.111 | −3.142 | 46.564 | 1.00 | 198.67 | O |
| ATOM | 9793 | CB | LYS | B | 2281 | −45.340 | −4.469 | 49.098 | 1.00 | 197.81 | C |
| ATOM | 9794 | CG | LYS | B | 2281 | −43.912 | −4.678 | 48.632 | 1.00 | 196.92 | C |
| ATOM | 9795 | CD | LYS | B | 2281 | −42.884 | −4.231 | 49.633 | 1.00 | 195.40 | C |
| ATOM | 9796 | CE | LYS | B | 2281 | −41.505 | −4.724 | 49.249 | 1.00 | 194.69 | C |
| ATOM | 9797 | NZ | LYS | B | 2281 | −40.579 | −4.744 | 50.410 | 1.00 | 193.93 | N |
| ATOM | 9798 | N | VAL | B | 2282 | −44.929 | −1.409 | 47.404 | 1.00 | 199.06 | N |
| ATOM | 9799 | CA | VAL | B | 2282 | −44.531 | −0.846 | 46.124 | 1.00 | 199.38 | C |
| ATOM | 9800 | C | VAL | B | 2282 | −43.360 | −1.682 | 45.645 | 1.00 | 199.62 | C |
| ATOM | 9801 | O | VAL | B | 2282 | −42.247 | −1.545 | 46.159 | 1.00 | 199.65 | O |
| ATOM | 9802 | CB | VAL | B | 2282 | −44.072 | 0.632 | 46.280 | 1.00 | 199.43 | C |
| ATOM | 9803 | CG1 | VAL | B | 2282 | −43.699 | 1.228 | 44.929 | 1.00 | 199.67 | C |
| ATOM | 9804 | CG2 | VAL | B | 2282 | −45.141 | 1.485 | 46.979 | 1.00 | 199.30 | C |
| ATOM | 9805 | N | PHE | B | 2283 | −43.608 | −2.564 | 44.681 | 1.00 | 199.97 | N |
| ATOM | 9806 | CA | PHE | B | 2283 | −42.585 | −3.533 | 44.275 | 1.00 | 200.49 | C |
| ATOM | 9807 | C | PHE | B | 2283 | −41.579 | −3.033 | 43.241 | 1.00 | 200.72 | C |
| ATOM | 9808 | O | PHE | B | 2283 | −41.952 | −2.437 | 42.228 | 1.00 | 200.77 | O |
| ATOM | 9809 | CB | PHE | B | 2283 | −43.209 | −4.856 | 43.832 | 1.00 | 200.51 | C |
| ATOM | 9810 | CG | PHE | B | 2283 | −43.465 | −5.805 | 44.965 | 1.00 | 200.95 | C |
| ATOM | 9811 | CD1 | PHE | B | 2283 | −44.763 | −6.102 | 45.361 | 1.00 | 201.66 | C |
| ATOM | 9812 | CD2 | PHE | B | 2283 | −42.404 | −6.391 | 45.655 | 1.00 | 201.13 | C |
| ATOM | 9813 | CE1 | PHE | B | 2283 | −44.999 | −6.979 | 46.427 | 1.00 | 201.70 | C |
| ATOM | 9814 | CE2 | PHE | B | 2283 | −42.631 | −7.271 | 46.721 | 1.00 | 200.90 | C |
| ATOM | 9815 | CZ | PHE | B | 2283 | −43.928 | −7.568 | 47.102 | 1.00 | 201.03 | C |
| ATOM | 9816 | N | GLN | B | 2284 | −40.301 | −3.293 | 43.515 | 1.00 | 200.99 | N |
| ATOM | 9817 | CA | GLN | B | 2284 | −39.223 | −2.855 | 42.637 | 1.00 | 201.27 | C |
| ATOM | 9818 | C | GLN | B | 2284 | −39.084 | −3.780 | 41.450 | 1.00 | 201.41 | C |
| ATOM | 9819 | O | GLN | B | 2284 | −38.492 | −4.850 | 41.561 | 1.00 | 201.53 | O |
| ATOM | 9820 | CB | GLN | B | 2284 | −37.896 | −2.732 | 43.393 | 1.00 | 201.29 | C |
| ATOM | 9821 | CG | GLN | B | 2284 | −37.770 | −1.451 | 44.227 | 1.00 | 201.48 | C |
| ATOM | 9822 | CD | GLN | B | 2284 | −38.111 | −0.155 | 43.462 | 1.00 | 201.14 | C |
| ATOM | 9823 | OE1 | GLN | B | 2284 | −38.359 | −0.161 | 42.252 | 1.00 | 200.87 | O |
| ATOM | 9824 | NE2 | GLN | B | 2284 | −38.121 | 0.962 | 44.183 | 1.00 | 200.97 | N |
| ATOM | 9825 | N | GLY | B | 2285 | −39.631 | −3.351 | 40.314 | 1.00 | 201.62 | N |
| ATOM | 9826 | CA | GLY | B | 2285 | −39.721 | −4.193 | 39.116 | 1.00 | 201.80 | C |
| ATOM | 9827 | C | GLY | B | 2285 | −38.655 | −4.006 | 38.044 | 1.00 | 201.78 | C |
| ATOM | 9828 | O | GLY | B | 2285 | −37.544 | −3.528 | 38.317 | 1.00 | 202.10 | O |
| ATOM | 9829 | N | ASN | B | 2286 | −39.014 | −4.387 | 36.819 | 1.00 | 201.37 | N |
| ATOM | 9830 | CA | ASN | B | 2286 | −38.089 | −4.445 | 35.694 | 1.00 | 200.93 | C |
| ATOM | 9831 | C | ASN | B | 2286 | −37.491 | −3.116 | 35.244 | 1.00 | 200.67 | C |
| ATOM | 9832 | O | ASN | B | 2286 | −37.773 | −2.052 | 35.812 | 1.00 | 200.56 | O |
| ATOM | 9833 | CB | ASN | B | 2286 | −38.803 | −5.051 | 34.492 | 1.00 | 200.97 | C |
| ATOM | 9834 | CG | ASN | B | 2286 | −39.085 | −6.502 | 34.661 | 1.00 | 200.69 | C |
| ATOM | 9835 | OD1 | ASN | B | 2286 | −38.169 | −7.318 | 34.792 | 1.00 | 200.52 | O |
| ATOM | 9836 | ND2 | ASN | B | 2286 | −40.363 | −6.849 | 34.632 | 1.00 | 200.38 | N |
| ATOM | 9837 | N | GLN | B | 2287 | −36.668 | −3.215 | 34.198 | 1.00 | 200.27 | N |
| ATOM | 9838 | CA | GLN | B | 2287 | −36.191 | −2.070 | 33.432 | 1.00 | 199.79 | C |
| ATOM | 9839 | C | GLN | B | 2287 | −35.808 | −2.407 | 31.975 | 1.00 | 199.38 | C |
| ATOM | 9840 | O | GLN | B | 2287 | −35.260 | −1.555 | 31.271 | 1.00 | 199.41 | O |
| ATOM | 9841 | CB | GLN | B | 2287 | −35.030 | −1.393 | 34.152 | 1.00 | 199.84 | C |
| ATOM | 9842 | CG | GLN | B | 2287 | −33.823 | −2.275 | 34.348 | 1.00 | 200.46 | C |
| ATOM | 9843 | CD | GLN | B | 2287 | −32.889 | −1.707 | 35.377 | 1.00 | 201.40 | C |
| ATOM | 9844 | OE1 | GLN | B | 2287 | −33.256 | −1.573 | 36.547 | 1.00 | 202.15 | O |
| ATOM | 9845 | NE2 | GLN | B | 2287 | −31.673 | −1.357 | 34.955 | 1.00 | 201.43 | N |
| ATOM | 9846 | N | ASP | B | 2288 | −36.105 | −3.629 | 31.521 | 1.00 | 198.88 | N |
| ATOM | 9847 | CA | ASP | B | 2288 | −35.783 | −4.045 | 30.141 | 1.00 | 198.35 | C |
| ATOM | 9848 | C | ASP | B | 2288 | −36.647 | −5.161 | 29.544 | 1.00 | 197.93 | C |
| ATOM | 9849 | O | ASP | B | 2288 | −37.349 | −5.880 | 30.253 | 1.00 | 197.58 | O |
| ATOM | 9850 | CB | ASP | B | 2288 | −34.285 | −4.373 | 29.979 | 1.00 | 198.42 | C |
| ATOM | 9851 | CG | ASP | B | 2288 | −33.715 | −5.154 | 31.153 | 1.00 | 198.43 | C |
| ATOM | 9852 | OD1 | ASP | B | 2288 | −32.480 | −5.355 | 31.182 | 1.00 | 198.10 | O |
| ATOM | 9853 | OD2 | ASP | B | 2288 | −34.492 | −5.560 | 32.047 | 1.00 | 198.71 | O |
| ATOM | 9854 | N | SER | B | 2289 | −36.579 | −5.270 | 28.218 | 1.00 | 197.72 | N |
| ATOM | 9855 | CA | SER | B | 2289 | −37.192 | −6.348 | 27.443 | 1.00 | 197.47 | C |
| ATOM | 9856 | C | SER | B | 2289 | −36.835 | −7.708 | 28.024 | 1.00 | 197.04 | C |
| ATOM | 9857 | O | SER | B | 2289 | −37.661 | −8.607 | 28.057 | 1.00 | 196.86 | O |
| ATOM | 9858 | CB | SER | B | 2289 | −36.705 | −6.299 | 25.980 | 1.00 | 197.74 | C |
| ATOM | 9859 | OG | SER | B | 2289 | −37.165 | −5.150 | 25.277 | 1.00 | 198.12 | O |
| ATOM | 9860 | N | PHE | B | 2290 | −35.584 | −7.842 | 28.455 | 1.00 | 196.68 | N |
| ATOM | 9861 | CA | PHE | B | 2290 | −35.071 | −9.066 | 29.066 | 1.00 | 196.39 | C |
| ATOM | 9862 | C | PHE | B | 2290 | −34.855 | −8.872 | 30.580 | 1.00 | 196.17 | C |
| ATOM | 9863 | O | PHE | B | 2290 | −35.605 | −8.114 | 31.216 | 1.00 | 196.38 | O |
| ATOM | 9864 | CB | PHE | B | 2290 | −33.791 | −9.533 | 28.353 | 1.00 | 196.35 | C |
| ATOM | 9865 | CG | PHE | B | 2290 | −32.815 | −8.419 | 28.036 | 1.00 | 196.45 | C |
| ATOM | 9866 | CD1 | PHE | B | 2290 | −31.662 | −8.249 | 28.799 | 1.00 | 196.27 | C |
| ATOM | 9867 | CD2 | PHE | B | 2290 | −33.039 | −7.553 | 26.960 | 1.00 | 196.74 | C |
| ATOM | 9868 | CE1 | PHE | B | 2290 | −30.750 | −7.232 | 28.506 | 1.00 | 196.21 | C |
| ATOM | 9869 | CE2 | PHE | B | 2290 | −32.134 | −6.529 | 26.660 | 1.00 | 196.63 | C |
| ATOM | 9870 | CZ | PHE | B | 2290 | −30.986 | −6.373 | 27.433 | 1.00 | 196.46 | C |
| ATOM | 9871 | N | THR | B | 2291 | −33.854 | −9.562 | 31.142 | 1.00 | 195.54 | N |
| ATOM | 9872 | CA | THR | B | 2291 | −33.468 | −9.478 | 32.568 | 1.00 | 194.75 | C |
| ATOM | 9873 | C | THR | B | 2291 | −34.614 | −9.690 | 33.558 | 1.00 | 194.20 | C |
| ATOM | 9874 | O | THR | B | 2291 | −35.597 | −8.951 | 33.575 | 1.00 | 194.02 | O |
| ATOM | 9875 | CB | THR | B | 2291 | −32.686 | −8.173 | 32.909 | 1.00 | 194.82 | C |
| ATOM | 9876 | OG1 | THR | B | 2291 | −31.636 | −7.972 | 31.956 | 1.00 | 194.92 | O |
| ATOM | 9877 | CG2 | THR | B | 2291 | −32.076 | −8.235 | 34.306 | 1.00 | 194.73 | C |
| ATOM | 9878 | N | PRO | B | 2292 | −34.476 | −10.712 | 34.394 | 1.00 | 193.84 | N |
| ATOM | 9879 | CA | PRO | B | 2292 | −35.478 | −11.034 | 35.374 | 1.00 | 193.88 | C |
| ATOM | 9880 | C | PRO | B | 2292 | −35.226 | −10.357 | 36.734 | 1.00 | 193.96 | C |
| ATOM | 9881 | O | PRO | B | 2292 | −34.175 | −9.742 | 36.932 | 1.00 | 193.79 | O |
| ATOM | 9882 | CB | PRO | B | 2292 | −35.357 | −12.553 | 35.473 | 1.00 | 193.89 | C |
| ATOM | 9883 | CG | PRO | B | 2292 | −33.947 | −12.868 | 35.077 | 1.00 | 193.82 | C |
| ATOM | 9884 | CD | PRO | B | 2292 | −33.347 | −11.651 | 34.445 | 1.00 | 193.80 | C |
| ATOM | 9885 | N | VAL | B | 2293 | −36.190 | −10.473 | 37.655 | 1.00 | 194.19 | N |
| ATOM | 9886 | CA | VAL | B | 2293 | −36.117 | −9.795 | 38.969 | 1.00 | 194.34 | C |
| ATOM | 9887 | C | VAL | B | 2293 | −36.291 | −10.681 | 40.261 | 1.00 | 194.43 | C |
| ATOM | 9888 | O | VAL | B | 2293 | −37.407 | −11.130 | 40.582 | 1.00 | 194.09 | O |
| ATOM | 9889 | CB | VAL | B | 2293 | −37.074 | −8.552 | 38.995 | 1.00 | 194.30 | C |
| ATOM | 9890 | CG1 | VAL | B | 2293 | −36.958 | −7.802 | 40.307 | 1.00 | 194.40 | C |
| ATOM | 9891 | CG2 | VAL | B | 2293 | −36.776 | −7.602 | 37.827 | 1.00 | 194.10 | C |
| ATOM | 9892 | N | VAL | B | 2294 | −35.173 | −10.926 | 40.975 | 1.00 | 194.53 | N |
| ATOM | 9893 | CA | VAL | B | 2294 | −35.169 | −11.508 | 42.344 | 1.00 | 194.33 | C |
| ATOM | 9894 | C | VAL | B | 2294 | −36.044 | −10.643 | 43.260 | 1.00 | 194.40 | C |
| ATOM | 9895 | O | VAL | B | 2294 | −35.534 | −9.901 | 44.115 | 1.00 | 194.65 | O |
| ATOM | 9896 | CB | VAL | B | 2294 | −33.709 | −11.637 | 43.018 | 1.00 | 194.29 | C |
| ATOM | 9897 | CG1 | VAL | B | 2294 | −33.134 | −13.041 | 42.903 | 1.00 | 193.91 | C |
| ATOM | 9898 | CG2 | VAL | B | 2294 | −32.709 | −10.560 | 42.538 | 1.00 | 194.15 | C |
| ATOM | 9899 | N | ASN | B | 2295 | −37.358 | −10.723 | 43.075 | 1.00 | 194.14 | N |
| ATOM | 9900 | CA | ASN | B | 2295 | −38.278 | −9.929 | 43.879 | 1.00 | 194.09 | C |
| ATOM | 9901 | C | ASN | B | 2295 | −39.060 | −10.856 | 44.819 | 1.00 | 194.05 | C |
| ATOM | 9902 | O | ASN | B | 2295 | −40.006 | −11.522 | 44.383 | 1.00 | 194.12 | O |
| ATOM | 9903 | CB | ASN | B | 2295 | −39.215 | −9.127 | 42.962 | 1.00 | 194.08 | C |
| ATOM | 9904 | CG | ASN | B | 2295 | −39.390 | −7.667 | 43.402 | 1.00 | 194.26 | C |
| ATOM | 9905 | OD1 | ASN | B | 2295 | −38.567 | −7.109 | 44.134 | 1.00 | 194.63 | O |
| ATOM | 9906 | ND2 | ASN | B | 2295 | −40.466 | −7.040 | 42.934 | 1.00 | 194.40 | N |
| ATOM | 9907 | N | SER | B | 2296 | −38.652 | −10.918 | 46.093 | 1.00 | 193.93 | N |
| ATOM | 9908 | CA | SER | B | 2296 | −39.255 | −11.862 | 47.060 | 1.00 | 193.84 | C |
| ATOM | 9909 | C | SER | B | 2296 | −40.011 | −11.198 | 48.215 | 1.00 | 193.73 | C |
| ATOM | 9910 | O | SER | B | 2296 | −39.997 | −9.972 | 48.356 | 1.00 | 193.80 | O |
| ATOM | 9911 | CB | SER | B | 2296 | −38.231 | −12.899 | 47.574 | 1.00 | 193.87 | C |
| ATOM | 9912 | OG | SER | B | 2296 | −37.273 | −12.328 | 48.448 | 1.00 | 193.98 | O |
| ATOM | 9913 | N | LEU | B | 2297 | −40.663 | −12.013 | 49.041 | 1.00 | 193.56 | N |
| ATOM | 9914 | CA | LEU | B | 2297 | −41.625 | −11.503 | 50.018 | 1.00 | 193.51 | C |
| ATOM | 9915 | C | LEU | B | 2297 | −41.179 | −11.633 | 51.484 | 1.00 | 193.52 | C |
| ATOM | 9916 | O | LEU | B | 2297 | −40.655 | −12.682 | 51.891 | 1.00 | 193.47 | O |
| ATOM | 9917 | CB | LEU | B | 2297 | −42.973 | −12.194 | 49.800 | 1.00 | 193.53 | C |
| ATOM | 9918 | CG | LEU | B | 2297 | −43.399 | −12.408 | 48.344 | 1.00 | 193.05 | C |
| ATOM | 9919 | CD1 | LEU | B | 2297 | −44.582 | −13.366 | 48.241 | 1.00 | 192.85 | C |
| ATOM | 9920 | CD2 | LEU | B | 2297 | −43.715 | −11.077 | 47.703 | 1.00 | 192.84 | C |
| ATOM | 9921 | N | ASP | B | 2298 | −41.399 | −10.570 | 52.268 | 1.00 | 193.42 | N |
| ATOM | 9922 | CA | ASP | B | 2298 | −40.980 | −10.534 | 53.684 | 1.00 | 193.37 | C |
| ATOM | 9923 | C | ASP | B | 2298 | −41.713 | −11.589 | 54.516 | 1.00 | 193.30 | C |
| ATOM | 9924 | O | ASP | B | 2298 | −41.065 | −12.468 | 55.093 | 1.00 | 193.44 | O |
| ATOM | 9925 | CB | ASP | B | 2298 | −41.111 | −9.122 | 54.302 | 1.00 | 193.36 | C |
| ATOM | 9926 | CG | ASP | B | 2298 | −40.473 | −9.014 | 55.703 | 1.00 | 193.14 | C |
| ATOM | 9927 | OD1 | ASP | B | 2298 | −41.225 | −8.939 | 56.697 | 1.00 | 193.08 | O |
| ATOM | 9928 | OD2 | ASP | B | 2298 | −39.226 | −8.998 | 55.818 | 1.00 | 192.65 | O |
| ATOM | 9929 | N | PRO | B | 2299 | −43.058 | −11.516 | 54.589 | 1.00 | 193.15 | N |
| ATOM | 9930 | CA | PRO | B | 2299 | −43.697 | −12.697 | 55.145 | 1.00 | 192.98 | C |
| ATOM | 9931 | C | PRO | B | 2299 | −43.992 | −13.679 | 54.010 | 1.00 | 192.64 | C |
| ATOM | 9932 | O | PRO | B | 2299 | −44.800 | −13.380 | 53.126 | 1.00 | 192.65 | O |
| ATOM | 9933 | CB | PRO | B | 2299 | −44.985 | −12.147 | 55.778 | 1.00 | 193.08 | C |
| ATOM | 9934 | CG | PRO | B | 2299 | −45.253 | −10.828 | 55.068 | 1.00 | 193.26 | C |
| ATOM | 9935 | CD | PRO | B | 2299 | −44.041 | −10.476 | 54.229 | 1.00 | 193.12 | C |
| ATOM | 9936 | N | PRO | B | 2300 | −43.320 | −14.840 | 54.014 | 1.00 | 192.29 | N |
| ATOM | 9937 | CA | PRO | B | 2300 | −43.610 | −15.818 | 52.978 | 1.00 | 191.99 | C |
| ATOM | 9938 | C | PRO | B | 2300 | −45.125 | −16.019 | 52.878 | 1.00 | 191.68 | C |
| ATOM | 9939 | O | PRO | B | 2300 | −45.762 | −16.406 | 53.853 | 1.00 | 191.93 | O |
| ATOM | 9940 | CB | PRO | B | 2300 | −42.924 | −17.080 | 53.502 | 1.00 | 192.14 | C |
| ATOM | 9941 | CG | PRO | B | 2300 | −41.792 | −16.571 | 54.344 | 1.00 | 192.32 | C |
| ATOM | 9942 | CD | PRO | B | 2300 | −42.301 | −15.315 | 54.972 | 1.00 | 192.34 | C |
| ATOM | 9943 | N | LEU | B | 2301 | −45.703 | −15.722 | 51.722 | 1.00 | 191.21 | N |
| ATOM | 9944 | CA | LEU | B | 2301 | −47.154 | −15.729 | 51.578 | 1.00 | 190.91 | C |
| ATOM | 9945 | C | LEU | B | 2301 | −47.720 | −17.136 | 51.360 | 1.00 | 190.65 | C |
| ATOM | 9946 | O | LEU | B | 2301 | −47.400 | −17.790 | 50.367 | 1.00 | 190.71 | O |
| ATOM | 9947 | CB | LEU | B | 2301 | −47.546 | −14.835 | 50.405 | 1.00 | 190.97 | C |
| ATOM | 9948 | CG | LEU | B | 2301 | −48.781 | −13.940 | 50.501 | 1.00 | 191.29 | C |
| ATOM | 9949 | CD1 | LEU | B | 2301 | −49.180 | −13.548 | 49.086 | 1.00 | 190.94 | C |
| ATOM | 9950 | CD2 | LEU | B | 2301 | −49.959 | −14.581 | 51.262 | 1.00 | 191.84 | C |
| ATOM | 9951 | N | LEU | B | 2302 | −48.564 | −17.601 | 52.277 | 1.00 | 190.29 | N |
| ATOM | 9952 | CA | LEU | B | 2302 | −49.234 | −18.887 | 52.086 | 1.00 | 190.03 | C |
| ATOM | 9953 | C | LEU | B | 2302 | −50.596 | −18.682 | 51.432 | 1.00 | 189.97 | C |
| ATOM | 9954 | O | LEU | B | 2302 | −51.508 | −18.106 | 52.036 | 1.00 | 190.03 | O |
| ATOM | 9955 | CB | LEU | B | 2302 | −49.352 | −19.670 | 53.397 | 1.00 | 189.95 | C |
| ATOM | 9956 | CG | LEU | B | 2302 | −48.137 | −20.490 | 53.853 | 1.00 | 189.87 | C |
| ATOM | 9957 | CD1 | LEU | B | 2302 | −47.171 | −19.672 | 54.704 | 1.00 | 189.53 | C |
| ATOM | 9958 | CD2 | LEU | B | 2302 | −48.583 | −21.726 | 54.622 | 1.00 | 189.83 | C |
| ATOM | 9959 | N | THR | B | 2303 | −50.711 | −19.150 | 50.187 | 1.00 | 189.88 | N |
| ATOM | 9960 | CA | THR | B | 2303 | −51.882 | −18.891 | 49.333 | 1.00 | 189.60 | C |
| ATOM | 9961 | C | THR | B | 2303 | −52.086 | −19.948 | 48.219 | 1.00 | 189.59 | C |
| ATOM | 9962 | O | THR | B | 2303 | −51.374 | −20.955 | 48.152 | 1.00 | 189.54 | O |
| ATOM | 9963 | CB | THR | B | 2303 | −51.854 | −17.431 | 48.761 | 1.00 | 189.42 | C |
| ATOM | 9964 | OG1 | THR | B | 2303 | −52.984 | −17.205 | 47.913 | 1.00 | 189.06 | O |
| ATOM | 9965 | CG2 | THR | B | 2303 | −50.585 | −17.174 | 47.980 | 1.00 | 189.20 | C |
| ATOM | 9966 | N | ARG | B | 2304 | −53.081 | −19.713 | 47.368 | 1.00 | 189.50 | N |
| ATOM | 9967 | CA | ARG | B | 2304 | −53.427 | −20.613 | 46.270 | 1.00 | 189.44 | C |
| ATOM | 9968 | C | ARG | B | 2304 | −53.867 | −19.772 | 45.071 | 1.00 | 189.11 | C |
| ATOM | 9969 | O | ARG | B | 2304 | −53.906 | −20.230 | 43.930 | 1.00 | 189.05 | O |
| ATOM | 9970 | CB | ARG | B | 2304 | −54.538 | −21.581 | 46.704 | 1.00 | 189.47 | C |
| ATOM | 9971 | CG | ARG | B | 2304 | −55.036 | −22.499 | 45.598 | 1.00 | 189.68 | C |
| ATOM | 9972 | CD | ARG | B | 2304 | −56.269 | −23.271 | 45.990 | 1.00 | 189.89 | C |
| ATOM | 9973 | NE | ARG | B | 2304 | −57.070 | −23.615 | 44.816 | 1.00 | 191.10 | N |
| ATOM | 9974 | CZ | ARG | B | 2304 | −58.108 | −24.450 | 44.824 | 1.00 | 192.09 | C |
| ATOM | 9975 | NH1 | ARG | B | 2304 | −58.489 | −25.060 | 45.947 | 1.00 | 192.33 | N |
| ATOM | 9976 | NH2 | ARG | B | 2304 | −58.768 | −24.686 | 43.697 | 1.00 | 192.48 | N |
| ATOM | 9977 | N | TYR | B | 2305 | −54.208 | −18.528 | 45.342 | 1.00 | 188.87 | N |
| ATOM | 9978 | CA | TYR | B | 2305 | −54.572 | −17.635 | 44.280 | 1.00 | 188.82 | C |
| ATOM | 9979 | C | TYR | B | 2305 | −53.712 | −16.388 | 44.371 | 1.00 | 189.06 | C |
| ATOM | 9980 | O | TYR | B | 2305 | −53.875 | −15.548 | 45.270 | 1.00 | 189.14 | O |
| ATOM | 9981 | CB | TYR | B | 2305 | −56.061 | −17.327 | 44.323 | 1.00 | 188.61 | C |
| ATOM | 9982 | CG | TYR | B | 2305 | −56.927 | −18.529 | 44.061 | 1.00 | 188.30 | C |
| ATOM | 9983 | CD1 | TYR | B | 2305 | −57.241 | −19.417 | 45.085 | 1.00 | 188.39 | C |
| ATOM | 9984 | CD2 | TYR | B | 2305 | −57.445 | −18.777 | 42.794 | 1.00 | 188.09 | C |
| ATOM | 9985 | CE1 | TYR | B | 2305 | −58.043 | −20.517 | 44.856 | 1.00 | 188.27 | C |
| ATOM | 9986 | CE2 | TYR | B | 2305 | −58.254 | −19.878 | 42.555 | 1.00 | 188.08 | C |
| ATOM | 9987 | CZ | TYR | B | 2305 | −58.541 | −20.745 | 43.593 | 1.00 | 188.20 | C |
| ATOM | 9988 | OH | TYR | B | 2305 | −59.332 | −21.842 | 43.381 | 1.00 | 188.27 | O |
| ATOM | 9989 | N | LEU | B | 2306 | −52.779 | −16.303 | 43.427 | 1.00 | 189.18 | N |
| ATOM | 9990 | CA | LEU | B | 2306 | −51.815 | −15.223 | 43.339 | 1.00 | 189.19 | C |
| ATOM | 9991 | C | LEU | B | 2306 | −52.100 | −14.301 | 42.143 | 1.00 | 189.43 | C |
| ATOM | 9992 | O | LEU | B | 2306 | −52.200 | −14.753 | 40.999 | 1.00 | 189.32 | O |
| ATOM | 9993 | CB | LEU | B | 2306 | −50.417 | −15.824 | 43.254 | 1.00 | 188.93 | C |
| ATOM | 9994 | CG | LEU | B | 2306 | −49.221 | −14.941 | 43.589 | 1.00 | 189.08 | C |
| ATOM | 9995 | CD1 | LEU | B | 2306 | −49.492 | −14.052 | 44.785 | 1.00 | 189.62 | C |
| ATOM | 9996 | CD2 | LEU | B | 2306 | −48.018 | −15.824 | 43.850 | 1.00 | 189.58 | C |
| ATOM | 9997 | N | ARG | B | 2307 | −52.240 | −13.008 | 42.424 | 1.00 | 189.82 | N |
| ATOM | 9998 | CA | ARG | B | 2307 | −52.549 | −12.015 | 41.407 | 1.00 | 190.30 | C |
| ATOM | 9999 | C | ARG | B | 2307 | −51.484 | −10.945 | 41.337 | 1.00 | 190.67 | C |
| ATOM | 10000 | O | ARG | B | 2307 | −51.123 | −10.362 | 42.356 | 1.00 | 190.61 | O |
| ATOM | 10001 | CB | ARG | B | 2307 | −53.855 | −11.325 | 41.731 | 1.00 | 190.28 | C |
| ATOM | 10002 | CG | ARG | B | 2307 | −55.073 | −12.038 | 41.304 | 1.00 | 191.06 | C |
| ATOM | 10003 | CD | ARG | B | 2307 | −56.204 | −11.058 | 41.331 | 1.00 | 193.10 | C |
| ATOM | 10004 | NE | ARG | B | 2307 | −57.415 | −11.625 | 40.756 | 1.00 | 195.24 | N |
| ATOM | 10005 | CZ | ARG | B | 2307 | −58.509 | −10.925 | 40.470 | 1.00 | 196.15 | C |
| ATOM | 10006 | NH1 | ARG | B | 2307 | −58.545 | −9.613 | 40.693 | 1.00 | 196.26 | N |
| ATOM | 10007 | NH2 | ARG | B | 2307 | −59.568 | −11.541 | 39.958 | 1.00 | 196.81 | N |
| ATOM | 10008 | N | ILE | B | 2308 | −51.007 | −10.674 | 40.126 | 1.00 | 191.35 | N |
| ATOM | 10009 | CA | ILE | B | 2308 | −50.034 | −9.610 | 39.879 | 1.00 | 192.07 | C |
| ATOM | 10010 | C | ILE | B | 2308 | −50.768 | −8.304 | 39.623 | 1.00 | 192.51 | C |
| ATOM | 10011 | O | ILE | B | 2308 | −51.711 | −8.266 | 38.827 | 1.00 | 192.45 | O |
| ATOM | 10012 | CB | ILE | B | 2308 | −49.149 | −9.914 | 38.642 | 1.00 | 192.08 | C |
| ATOM | 10013 | CG1 | ILE | B | 2308 | −48.175 | −11.072 | 38.903 | 1.00 | 192.16 | C |
| ATOM | 10014 | CG2 | ILE | B | 2308 | −48.380 | −8.676 | 38.214 | 1.00 | 192.65 | C |
| ATOM | 10015 | CD1 | ILE | B | 2308 | −46.877 | −10.693 | 39.611 | 1.00 | 192.22 | C |
| ATOM | 10016 | N | HIS | B | 2309 | −50.321 | −7.236 | 40.279 | 1.00 | 193.19 | N |
| ATOM | 10017 | CA | HIS | B | 2309 | −50.939 | −5.923 | 40.105 | 1.00 | 194.02 | C |
| ATOM | 10018 | C | HIS | B | 2309 | −49.962 | −4.848 | 39.630 | 1.00 | 194.12 | C |
| ATOM | 10019 | O | HIS | B | 2309 | −49.349 | −4.159 | 40.450 | 1.00 | 194.36 | O |
| ATOM | 10020 | CB | HIS | B | 2309 | −51.660 | −5.501 | 41.380 | 1.00 | 194.20 | C |
| ATOM | 10021 | CG | HIS | B | 2309 | −52.798 | −6.401 | 41.731 | 1.00 | 196.00 | C |
| ATOM | 10022 | ND1 | HIS | B | 2309 | −54.101 | −6.122 | 41.378 | 1.00 | 197.84 | N |
| ATOM | 10023 | CD2 | HIS | B | 2309 | −52.827 | −7.600 | 42.362 | 1.00 | 197.60 | C |
| ATOM | 10024 | CE1 | HIS | B | 2309 | −54.888 | −7.099 | 41.797 | 1.00 | 198.63 | C |
| ATOM | 10025 | NE2 | HIS | B | 2309 | −54.139 | −8.010 | 42.396 | 1.00 | 198.49 | N |
| ATOM | 10026 | N | PRO | B | 2310 | −49.823 | −4.702 | 38.295 | 1.00 | 194.08 | N |
| ATOM | 10027 | CA | PRO | B | 2310 | −48.962 | −3.696 | 37.683 | 1.00 | 194.00 | C |
| ATOM | 10028 | C | PRO | B | 2310 | −49.350 | −2.267 | 38.056 | 1.00 | 194.04 | C |
| ATOM | 10029 | O | PRO | B | 2310 | −50.521 | −1.892 | 37.989 | 1.00 | 193.89 | O |
| ATOM | 10030 | CB | PRO | B | 2310 | −49.164 | −3.932 | 36.182 | 1.00 | 193.98 | C |
| ATOM | 10031 | CG | PRO | B | 2310 | −50.446 | −4.673 | 36.068 | 1.00 | 193.85 | C |
| ATOM | 10032 | CD | PRO | B | 2310 | −50.495 | −5.528 | 37.276 | 1.00 | 194.01 | C |
| ATOM | 10033 | N | GLN | B | 2311 | −48.359 | −1.484 | 38.453 | 1.00 | 194.21 | N |
| ATOM | 10034 | CA | GLN | B | 2311 | −48.590 | −0.103 | 38.793 | 1.00 | 194.68 | C |
| ATOM | 10035 | C | GLN | B | 2311 | −48.004 | 0.829 | 37.756 | 1.00 | 194.84 | C |
| ATOM | 10036 | O | GLN | B | 2311 | −48.732 | 1.552 | 37.088 | 1.00 | 194.83 | O |
| ATOM | 10037 | CB | GLN | B | 2311 | −48.015 | 0.200 | 40.168 | 1.00 | 194.80 | C |
| ATOM | 10038 | CG | GLN | B | 2311 | −48.885 | −0.281 | 41.311 | 1.00 | 195.96 | C |
| ATOM | 10039 | CD | GLN | B | 2311 | −50.224 | 0.449 | 41.394 | 1.00 | 197.55 | C |
| ATOM | 10040 | OE1 | GLN | B | 2311 | −50.442 | 1.292 | 42.277 | 1.00 | 197.90 | O |
| ATOM | 10041 | NE2 | GLN | B | 2311 | −51.128 | 0.128 | 40.469 | 1.00 | 198.37 | N |
| ATOM | 10042 | N | SER | B | 2312 | −46.684 | 0.818 | 37.633 | 1.00 | 195.21 | N |
| ATOM | 10043 | CA | SER | B | 2312 | −46.009 | 1.652 | 36.659 | 1.00 | 195.72 | C |
| ATOM | 10044 | C | SER | B | 2312 | −45.241 | 0.802 | 35.693 | 1.00 | 195.85 | C |
| ATOM | 10045 | O | SER | B | 2312 | −44.713 | −0.242 | 36.053 | 1.00 | 195.85 | O |
| ATOM | 10046 | CB | SER | B | 2312 | −45.064 | 2.634 | 37.339 | 1.00 | 195.91 | C |
| ATOM | 10047 | OG | SER | B | 2312 | −45.715 | 3.876 | 37.560 | 1.00 | 196.95 | O |
| ATOM | 10048 | N | TRP | B | 2313 | −45.172 | 1.267 | 34.457 | 1.00 | 196.27 | N |
| ATOM | 10049 | CA | TRP | B | 2313 | −44.576 | 0.484 | 33.390 | 1.00 | 196.64 | C |
| ATOM | 10050 | C | TRP | B | 2313 | −43.750 | 1.352 | 32.435 | 1.00 | 196.66 | C |
| ATOM | 10051 | O | TRP | B | 2313 | −43.503 | 2.537 | 32.722 | 1.00 | 196.94 | O |
| ATOM | 10052 | CB | TRP | B | 2313 | −45.674 | −0.281 | 32.645 | 1.00 | 196.92 | C |
| ATOM | 10053 | CG | TRP | B | 2313 | −46.809 | 0.583 | 32.140 | 1.00 | 197.21 | C |
| ATOM | 10054 | CD1 | TRP | B | 2313 | −46.856 | 1.248 | 30.955 | 1.00 | 197.41 | C |
| ATOM | 10055 | CD2 | TRP | B | 2313 | −48.057 | 0.853 | 32.800 | 1.00 | 197.49 | C |
| ATOM | 10056 | NE1 | TRP | B | 2313 | −48.046 | 1.919 | 30.832 | 1.00 | 197.63 | N |
| ATOM | 10057 | CE2 | TRP | B | 2313 | −48.805 | 1.693 | 31.947 | 1.00 | 197.37 | C |
| ATOM | 10058 | CE3 | TRP | B | 2313 | −48.617 | 0.463 | 34.023 | 1.00 | 197.70 | C |
| ATOM | 10059 | CZ2 | TRP | B | 2313 | −50.081 | 2.154 | 32.275 | 1.00 | 197.09 | C |
| ATOM | 10060 | CZ3 | TRP | B | 2313 | −49.890 | 0.919 | 34.346 | 1.00 | 197.49 | C |
| ATOM | 10061 | CH2 | TRP | B | 2313 | −50.604 | 1.759 | 33.474 | 1.00 | 197.28 | C |
| ATOM | 10062 | N | VAL | B | 2314 | −43.330 | 0.764 | 31.309 | 1.00 | 196.38 | N |
| ATOM | 10063 | CA | VAL | B | 2314 | −42.490 | 1.469 | 30.335 | 1.00 | 196.07 | C |
| ATOM | 10064 | C | VAL | B | 2314 | −43.226 | 2.034 | 29.103 | 1.00 | 195.94 | C |
| ATOM | 10065 | O | VAL | B | 2314 | −43.413 | 3.249 | 29.010 | 1.00 | 196.21 | O |
| ATOM | 10066 | CB | VAL | B | 2314 | −41.278 | 0.638 | 29.907 | 1.00 | 196.02 | C |
| ATOM | 10067 | CG1 | VAL | B | 2314 | −41.708 | −0.533 | 29.030 | 1.00 | 195.95 | C |
| ATOM | 10068 | CG2 | VAL | B | 2314 | −40.262 | 1.534 | 29.202 | 1.00 | 195.88 | C |
| ATOM | 10069 | N | HIS | B | 2315 | −43.623 | 1.169 | 28.168 | 1.00 | 195.52 | N |
| ATOM | 10070 | CA | HIS | B | 2315 | −44.302 | 1.613 | 26.953 | 1.00 | 195.25 | C |
| ATOM | 10071 | C | HIS | B | 2315 | −45.724 | 1.078 | 26.901 | 1.00 | 195.20 | C |
| ATOM | 10072 | O | HIS | B | 2315 | −46.665 | 1.810 | 26.578 | 1.00 | 195.18 | O |
| ATOM | 10073 | CB | HIS | B | 2315 | −43.528 | 1.182 | 25.714 | 1.00 | 195.24 | C |
| ATOM | 10074 | CG | HIS | B | 2315 | −42.193 | 1.849 | 25.566 | 1.00 | 195.30 | C |
| ATOM | 10075 | ND1 | HIS | B | 2315 | −42.057 | 3.193 | 25.290 | 1.00 | 195.16 | N |
| ATOM | 10076 | CD2 | HIS | B | 2315 | −40.935 | 1.349 | 25.627 | 1.00 | 195.42 | C |
| ATOM | 10077 | CE1 | HIS | B | 2315 | −40.773 | 3.495 | 25.201 | 1.00 | 195.21 | C |
| ATOM | 10078 | NE2 | HIS | B | 2315 | −40.071 | 2.393 | 25.400 | 1.00 | 195.38 | N |
| ATOM | 10079 | N | GLN | B | 2316 | −45.855 | −0.212 | 27.198 | 1.00 | 195.19 | N |
| ATOM | 10080 | CA | GLN | B | 2316 | −47.137 | −0.861 | 27.446 | 1.00 | 195.26 | C |
| ATOM | 10081 | C | GLN | B | 2316 | −46.901 | −1.783 | 28.619 | 1.00 | 195.04 | C |
| ATOM | 10082 | O | GLN | B | 2316 | −45.749 | −2.073 | 28.964 | 1.00 | 195.23 | O |
| ATOM | 10083 | CB | GLN | B | 2316 | −47.577 | −1.719 | 26.259 | 1.00 | 195.36 | C |
| ATOM | 10084 | CG | GLN | B | 2316 | −47.287 | −1.136 | 24.889 | 1.00 | 196.52 | C |
| ATOM | 10085 | CD | GLN | B | 2316 | −48.433 | −0.313 | 24.342 | 1.00 | 198.13 | C |
| ATOM | 10086 | OE1 | GLN | B | 2316 | −49.605 | −0.652 | 24.545 | 1.00 | 198.65 | O |
| ATOM | 10087 | NE2 | GLN | B | 2316 | −48.103 | 0.771 | 23.628 | 1.00 | 198.43 | N |
| ATOM | 10088 | N | ILE | B | 2317 | −47.981 | −2.268 | 29.218 | 1.00 | 194.67 | N |
| ATOM | 10089 | CA | ILE | B | 2317 | −47.870 | −3.275 | 30.263 | 1.00 | 194.25 | C |
| ATOM | 10090 | C | ILE | B | 2317 | −47.612 | −4.631 | 29.604 | 1.00 | 194.11 | C |
| ATOM | 10091 | O | ILE | B | 2317 | −48.417 | −5.095 | 28.786 | 1.00 | 194.01 | O |
| ATOM | 10092 | CB | ILE | B | 2317 | −49.126 | −3.308 | 31.144 | 1.00 | 194.22 | C |
| ATOM | 10093 | CG1 | ILE | B | 2317 | −49.536 | −1.880 | 31.510 | 1.00 | 194.20 | C |
| ATOM | 10094 | CG2 | ILE | B | 2317 | −48.865 | −4.111 | 32.400 | 1.00 | 194.02 | C |
| ATOM | 10095 | CD1 | ILE | B | 2317 | −50.993 | −1.700 | 31.857 | 1.00 | 194.25 | C |
| ATOM | 10096 | N | ALA | B | 2318 | −46.478 | −5.241 | 29.956 | 1.00 | 193.95 | N |
| ATOM | 10097 | CA | ALA | B | 2318 | −45.998 | −6.473 | 29.316 | 1.00 | 193.92 | C |
| ATOM | 10098 | C | ALA | B | 2318 | −45.330 | −7.433 | 30.296 | 1.00 | 193.91 | C |
| ATOM | 10099 | O | ALA | B | 2318 | −44.355 | −7.076 | 30.957 | 1.00 | 193.91 | O |
| ATOM | 10100 | CB | ALA | B | 2318 | −45.032 | −6.125 | 28.213 | 1.00 | 194.00 | C |
| ATOM | 10101 | N | LEU | B | 2319 | −45.823 | −8.663 | 30.376 | 1.00 | 193.92 | N |
| ATOM | 10102 | CA | LEU | B | 2319 | −45.307 | −9.545 | 31.404 | 1.00 | 194.14 | C |
| ATOM | 10103 | C | LEU | B | 2319 | −44.993 | −10.973 | 30.993 | 1.00 | 194.30 | C |
| ATOM | 10104 | O | LEU | B | 2319 | −45.865 | −11.718 | 30.551 | 1.00 | 194.18 | O |
| ATOM | 10105 | CB | LEU | B | 2319 | −46.222 | −9.521 | 32.631 | 1.00 | 194.16 | C |
| ATOM | 10106 | CG | LEU | B | 2319 | −45.591 | −9.950 | 33.961 | 1.00 | 194.51 | C |
| ATOM | 10107 | CD1 | LEU | B | 2319 | −44.362 | −9.119 | 34.292 | 1.00 | 194.95 | C |
| ATOM | 10108 | CD2 | LEU | B | 2319 | −46.597 | −9.877 | 35.099 | 1.00 | 194.24 | C |
| ATOM | 10109 | N | ARG | B | 2320 | −43.713 | −11.310 | 31.126 | 1.00 | 194.66 | N |
| ATOM | 10110 | CA | ARG | B | 2320 | −43.224 | −12.677 | 31.199 | 1.00 | 195.06 | C |
| ATOM | 10111 | C | ARG | B | 2320 | −42.781 | −12.819 | 32.637 | 1.00 | 195.56 | C |
| ATOM | 10112 | O | ARG | B | 2320 | −42.058 | −11.958 | 33.140 | 1.00 | 195.55 | O |
| ATOM | 10113 | CB | ARG | B | 2320 | −41.995 | −12.858 | 30.314 | 1.00 | 194.89 | C |
| ATOM | 10114 | CG | ARG | B | 2320 | −42.263 | −13.216 | 28.877 | 1.00 | 194.85 | C |
| ATOM | 10115 | CD | ARG | B | 2320 | −40.983 | −13.647 | 28.180 | 1.00 | 194.86 | C |
| ATOM | 10116 | NE | ARG | B | 2320 | −40.326 | −12.546 | 27.482 | 1.00 | 195.53 | N |
| ATOM | 10117 | CZ | ARG | B | 2320 | −39.084 | −12.585 | 27.001 | 1.00 | 196.37 | C |
| ATOM | 10118 | NH1 | ARG | B | 2320 | −38.325 | −13.672 | 27.153 | 1.00 | 196.36 | N |
| ATOM | 10119 | NH2 | ARG | B | 2320 | −38.592 | −11.524 | 26.369 | 1.00 | 196.70 | N |
| ATOM | 10120 | N | MET | B | 2321 | −43.197 | −13.890 | 33.308 | 1.00 | 196.24 | N |
| ATOM | 10121 | CA | MET | B | 2321 | −42.901 | −14.032 | 34.747 | 1.00 | 196.84 | C |
| ATOM | 10122 | C | MET | B | 2321 | −42.719 | −15.464 | 35.268 | 1.00 | 196.87 | C |
| ATOM | 10123 | O | MET | B | 2321 | −42.685 | −16.420 | 34.493 | 1.00 | 196.95 | O |
| ATOM | 10124 | CB | MET | B | 2321 | −43.960 | −13.304 | 35.581 | 1.00 | 196.84 | C |
| ATOM | 10125 | CG | MET | B | 2321 | −45.364 | −13.876 | 35.422 | 1.00 | 197.31 | C |
| ATOM | 10126 | SD | MET | B | 2321 | −46.377 | −13.697 | 36.901 | 1.00 | 197.43 | S |
| ATOM | 10127 | CE | MET | B | 2321 | −45.555 | −14.812 | 38.041 | 1.00 | 197.35 | C |
| ATOM | 10128 | N | GLU | B | 2322 | −42.609 | −15.587 | 36.591 | 1.00 | 196.99 | N |
| ATOM | 10129 | CA | GLU | B | 2322 | −42.380 | −16.858 | 37.268 | 1.00 | 197.25 | C |
| ATOM | 10130 | C | GLU | B | 2322 | −42.582 | −16.661 | 38.771 | 1.00 | 197.52 | C |
| ATOM | 10131 | O | GLU | B | 2322 | −42.312 | −15.574 | 39.283 | 1.00 | 197.40 | O |
| ATOM | 10132 | CB | GLU | B | 2322 | −40.957 | −17.336 | 36.974 | 1.00 | 197.29 | C |
| ATOM | 10133 | CG | GLU | B | 2322 | −40.380 | −18.376 | 37.928 | 1.00 | 197.20 | C |
| ATOM | 10134 | CD | GLU | B | 2322 | −40.190 | −19.731 | 37.291 | 1.00 | 196.46 | C |
| ATOM | 10135 | OE1 | GLU | B | 2322 | −39.032 | −20.047 | 36.902 | 1.00 | 195.68 | O |
| ATOM | 10136 | OE2 | GLU | B | 2322 | −41.199 | −20.465 | 37.189 | 1.00 | 195.91 | O |
| ATOM | 10137 | N | VAL | B | 2323 | −43.069 | −17.703 | 39.459 | 1.00 | 197.88 | N |
| ATOM | 10138 | CA | VAL | B | 2323 | −43.214 | −17.725 | 40.935 | 1.00 | 198.10 | C |
| ATOM | 10139 | C | VAL | B | 2323 | −42.182 | −18.689 | 41.536 | 1.00 | 198.54 | C |
| ATOM | 10140 | O | VAL | B | 2323 | −41.854 | −19.706 | 40.931 | 1.00 | 198.61 | O |
| ATOM | 10141 | CB | VAL | B | 2323 | −44.645 | −18.176 | 41.412 | 1.00 | 197.86 | C |
| ATOM | 10142 | CG1 | VAL | B | 2323 | −44.871 | −17.817 | 42.859 | 1.00 | 197.48 | C |
| ATOM | 10143 | CG2 | VAL | B | 2323 | −45.743 | −17.555 | 40.582 | 1.00 | 197.57 | C |
| ATOM | 10144 | N | LEU | B | 2324 | −41.670 | −18.361 | 42.717 | 1.00 | 199.09 | N |
| ATOM | 10145 | CA | LEU | B | 2324 | −40.798 | −19.267 | 43.450 | 1.00 | 199.76 | C |
| ATOM | 10146 | C | LEU | B | 2324 | −41.539 | −19.883 | 44.630 | 1.00 | 200.49 | C |
| ATOM | 10147 | O | LEU | B | 2324 | −42.282 | −19.188 | 45.324 | 1.00 | 200.57 | O |
| ATOM | 10148 | CB | LEU | B | 2324 | −39.584 | −18.513 | 43.975 | 1.00 | 199.61 | C |
| ATOM | 10149 | CG | LEU | B | 2324 | −38.425 | −18.180 | 43.049 | 1.00 | 199.24 | C |
| ATOM | 10150 | CD1 | LEU | B | 2324 | −37.344 | −17.491 | 43.865 | 1.00 | 199.65 | C |
| ATOM | 10151 | CD2 | LEU | B | 2324 | −37.879 | −19.430 | 42.410 | 1.00 | 198.42 | C |
| ATOM | 10152 | N | GLY | B | 2325 | −41.334 | −21.178 | 44.869 | 1.00 | 201.32 | N |
| ATOM | 10153 | CA | GLY | B | 2325 | −41.910 | −21.812 | 46.054 | 1.00 | 202.52 | C |
| ATOM | 10154 | C | GLY | B | 2325 | −42.211 | −23.303 | 46.036 | 1.00 | 203.50 | C |
| ATOM | 10155 | O | GLY | B | 2325 | −41.620 | −24.069 | 45.272 | 1.00 | 203.51 | O |
| ATOM | 10156 | N | CYS | B | 2326 | −43.142 | −23.697 | 46.906 | 1.00 | 204.58 | N |
| ATOM | 10157 | CA | CYS | B | 2326 | −43.498 | −25.093 | 47.173 | 1.00 | 205.82 | C |
| ATOM | 10158 | C | CYS | B | 2326 | −44.913 | −25.135 | 47.769 | 1.00 | 206.48 | C |
| ATOM | 10159 | O | CYS | B | 2326 | −45.796 | −24.452 | 47.259 | 1.00 | 206.64 | O |
| ATOM | 10160 | CB | CYS | B | 2326 | −42.481 | −25.700 | 48.133 | 1.00 | 205.90 | C |
| ATOM | 10161 | SG | CYS | B | 2326 | −42.228 | −24.696 | 49.627 | 1.00 | 207.06 | S |
| ATOM | 10162 | N | GLU | B | 2327 | −45.134 | −25.922 | 48.831 | 1.00 | 207.44 | N |
| ATOM | 10163 | CA | GLU | B | 2327 | −46.451 | −25.973 | 49.526 | 1.00 | 208.34 | C |
| ATOM | 10164 | C | GLU | B | 2327 | −46.433 | −26.502 | 50.968 | 1.00 | 208.90 | C |
| ATOM | 10165 | O | GLU | B | 2327 | −45.736 | −27.465 | 51.280 | 1.00 | 208.92 | O |
| ATOM | 10166 | CB | GLU | B | 2327 | −47.500 | −26.763 | 48.712 | 1.00 | 208.34 | C |
| ATOM | 10167 | CG | GLU | B | 2327 | −47.212 | −28.249 | 48.518 | 1.00 | 208.19 | C |
| ATOM | 10168 | CD | GLU | B | 2327 | −45.905 | −28.491 | 47.788 | 1.00 | 207.88 | C |
| ATOM | 10169 | OE1 | GLU | B | 2327 | −45.101 | −29.307 | 48.271 | 1.00 | 207.88 | O |
| ATOM | 10170 | OE2 | GLU | B | 2327 | −45.667 | −27.843 | 46.748 | 1.00 | 207.55 | O |
| ATOM | 10171 | N | ALA | B | 2328 | −47.219 | −25.870 | 51.837 | 1.00 | 209.65 | N |
| ATOM | 10172 | CA | ALA | B | 2328 | −47.504 | −26.433 | 53.149 | 1.00 | 210.38 | C |
| ATOM | 10173 | C | ALA | B | 2328 | −48.683 | −27.385 | 52.973 | 1.00 | 210.95 | C |
| ATOM | 10174 | O | ALA | B | 2328 | −48.877 | −27.923 | 51.876 | 1.00 | 210.91 | O |
| ATOM | 10175 | CB | ALA | B | 2328 | −47.818 | −25.338 | 54.152 | 1.00 | 210.33 | C |
| ATOM | 10176 | N | GLN | B | 2329 | −49.461 | −27.587 | 54.041 | 1.00 | 211.75 | N |
| ATOM | 10177 | CA | GLN | B | 2329 | −50.684 | −28.415 | 54.020 | 1.00 | 212.52 | C |
| ATOM | 10178 | C | GLN | B | 2329 | −51.576 | −28.098 | 52.818 | 1.00 | 213.09 | C |
| ATOM | 10179 | O | GLN | B | 2329 | −51.414 | −27.045 | 52.177 | 1.00 | 213.13 | O |
| ATOM | 10180 | CB | GLN | B | 2329 | −51.480 | −28.237 | 55.320 | 1.00 | 212.41 | C |
| ATOM | 10181 | CG | GLN | B | 2329 | −51.632 | −26.783 | 55.787 | 1.00 | 212.85 | C |
| ATOM | 10182 | CD | GLN | B | 2329 | −52.108 | −25.834 | 54.684 | 1.00 | 213.22 | C |
| ATOM | 10183 | OE1 | GLN | B | 2329 | −53.265 | −25.882 | 54.261 | 1.00 | 213.44 | O |
| ATOM | 10184 | NE2 | GLN | B | 2329 | −51.207 | −24.977 | 54.206 | 1.00 | 213.04 | N |
| ATOM | 10185 | N | ASP | B | 2330 | −52.519 | −28.991 | 52.510 | 1.00 | 213.77 | N |
| ATOM | 10186 | CA | ASP | B | 2330 | −53.374 | −28.764 | 51.338 | 1.00 | 214.46 | C |
| ATOM | 10187 | C | ASP | B | 2330 | −54.762 | −28.209 | 51.685 | 1.00 | 214.55 | C |
| ATOM | 10188 | O | ASP | B | 2330 | −55.674 | −28.946 | 52.083 | 1.00 | 214.49 | O |
| ATOM | 10189 | CB | ASP | B | 2330 | −53.427 | −29.981 | 50.390 | 1.00 | 214.68 | C |
| ATOM | 10190 | CG | ASP | B | 2330 | −53.166 | −29.595 | 48.911 | 1.00 | 215.35 | C |
| ATOM | 10191 | OD1 | ASP | B | 2330 | −52.219 | −28.811 | 48.627 | 1.00 | 215.51 | O |
| ATOM | 10192 | OD2 | ASP | B | 2330 | −53.906 | −30.091 | 48.029 | 1.00 | 216.05 | O |
| ATOM | 10193 | N | LEU | B | 2331 | −54.865 | −26.887 | 51.535 | 1.00 | 214.74 | N |
| ATOM | 10194 | CA | LEU | B | 2331 | −56.084 | −26.083 | 51.719 | 1.00 | 214.86 | C |
| ATOM | 10195 | C | LEU | B | 2331 | −56.533 | −25.862 | 53.174 | 1.00 | 214.88 | C |
| ATOM | 10196 | O | LEU | B | 2331 | −56.850 | −24.734 | 53.546 | 1.00 | 214.80 | O |
| ATOM | 10197 | CB | LEU | B | 2331 | −57.238 | −26.550 | 50.803 | 1.00 | 214.91 | C |
| ATOM | 10198 | CG | LEU | B | 2331 | −57.404 | −26.092 | 49.333 | 1.00 | 214.87 | C |
| ATOM | 10199 | CD1 | LEU | B | 2331 | −57.498 | −24.564 | 49.181 | 1.00 | 214.56 | C |
| ATOM | 10200 | CD2 | LEU | B | 2331 | −56.345 | −26.693 | 48.388 | 1.00 | 214.41 | C |
| ATOM | 10201 | N | TYR | B | 2332 | −56.546 | −26.919 | 53.985 | 1.00 | 215.02 | N |
| ATOM | 10202 | CA | TYR | B | 2332 | −57.030 | −26.843 | 55.372 | 1.00 | 215.21 | C |
| ATOM | 10203 | C | TYR | B | 2332 | −56.430 | −27.948 | 56.224 | 1.00 | 215.15 | C |
| ATOM | 10204 | O | TYR | B | 2332 | −56.786 | −29.119 | 56.073 | 1.00 | 215.13 | O |
| ATOM | 10205 | CB | TYR | B | 2332 | −58.556 | −26.941 | 55.415 | 1.00 | 215.51 | C |
| ATOM | 10206 | CG | TYR | B | 2332 | −59.113 | −27.736 | 54.260 | 1.00 | 216.02 | C |
| ATOM | 10207 | CD1 | TYR | B | 2332 | −59.230 | −29.129 | 54.332 | 1.00 | 216.47 | C |
| ATOM | 10208 | CD2 | TYR | B | 2332 | −59.497 | −27.096 | 53.077 | 1.00 | 216.16 | C |
| ATOM | 10209 | CE1 | TYR | B | 2332 | −59.727 | −29.862 | 53.256 | 1.00 | 216.92 | C |
| ATOM | 10210 | CE2 | TYR | B | 2332 | −59.991 | −27.811 | 51.996 | 1.00 | 216.48 | C |
| ATOM | 10211 | CZ | TYR | B | 2332 | −60.104 | −29.192 | 52.088 | 1.00 | 216.82 | C |
| ATOM | 10212 | OH | TYR | B | 2332 | −60.600 | −29.896 | 51.012 | 1.00 | 216.85 | O |
| TER | 10213 | | TYR | B | 2332 | | | | | | |
| HETATM | 10214 | C1 | NAG | A | 755 | −55.568 | −42.329 | 100.234 | 1.00 | 210.57 | C |
| HETATM | 10215 | C2 | NAG | A | 755 | −54.707 | −43.247 | 101.113 | 1.00 | 215.64 | C |
| HETATM | 10216 | C3 | NAG | A | 755 | −54.463 | −42.683 | 102.523 | 1.00 | 216.97 | C |
| HETATM | 10217 | C4 | NAG | A | 755 | −54.245 | −41.165 | 102.597 | 1.00 | 218.43 | C |
| HETATM | 10218 | C5 | NAG | A | 755 | −55.186 | −40.431 | 101.622 | 1.00 | 216.42 | C |
| HETATM | 10219 | C6 | NAG | A | 755 | −54.844 | −38.951 | 101.474 | 1.00 | 216.54 | C |
| HETATM | 10220 | C7 | NAG | A | 755 | −54.671 | −45.752 | 101.102 | 1.00 | 217.94 | C |
| HETATM | 10221 | C8 | NAG | A | 755 | −54.744 | −46.488 | 99.761 | 1.00 | 217.82 | C |
| HETATM | 10222 | N2 | NAG | A | 755 | −55.338 | −44.565 | 101.204 | 1.00 | 216.83 | N |
| HETATM | 10223 | O3 | NAG | A | 755 | −53.331 | −43.328 | 103.072 | 1.00 | 217.19 | O |
| HETATM | 10224 | O4 | NAG | A | 755 | −54.494 | −40.718 | 103.931 | 1.00 | 222.89 | O |
| HETATM | 10225 | O5 | NAG | A | 755 | −55.142 | −40.986 | 100.319 | 1.00 | 213.35 | O |
| HETATM | 10226 | O6 | NAG | A | 755 | −54.851 | −38.312 | 102.731 | 1.00 | 216.78 | O |
| HETATM | 10227 | O7 | NAG | A | 755 | −54.028 | −46.273 | 102.055 | 1.00 | 218.73 | O |
| HETATM | 10228 | C1 | NAG | A | 756 | −53.362 | −40.511 | 104.851 | 1.00 | 227.13 | C |
| HETATM | 10229 | C2 | NAG | A | 756 | −53.485 | −41.394 | 106.130 | 1.00 | 229.01 | C |
| HETATM | 10230 | C3 | NAG | A | 756 | −52.149 | −41.848 | 106.798 | 1.00 | 229.51 | C |
| HETATM | 10231 | C4 | NAG | A | 756 | −50.823 | −41.255 | 106.275 | 1.00 | 229.51 | C |
| HETATM | 10232 | C5 | NAG | A | 756 | −51.031 | −40.130 | 105.248 | 1.00 | 229.44 | C |
| HETATM | 10233 | C6 | NAG | A | 756 | −49.720 | −39.827 | 104.509 | 1.00 | 229.62 | C |
| HETATM | 10234 | C7 | NAG | A | 756 | −55.743 | −40.701 | 107.080 | 1.00 | 230.74 | C |
| HETATM | 10235 | C8 | NAG | A | 756 | −56.384 | −39.656 | 107.705 | 1.00 | 230.47 | C |
| HETATM | 10236 | N2 | NAG | A | 756 | −54.385 | −40.779 | 107.142 | 1.00 | 230.13 | N |
| HETATM | 10237 | O3 | NAG | A | 756 | −52.019 | −43.265 | 106.771 | 1.00 | 229.69 | O |
| HETATM | 10238 | O4 | NAG | A | 756 | −50.035 | −40.810 | 107.368 | 1.00 | 229.34 | O |
| HETATM | 10239 | O5 | NAG | A | 756 | −52.037 | −40.504 | 104.306 | 1.00 | 228.64 | O |
| HETATM | 10240 | O6 | NAG | A | 756 | −49.079 | −38.719 | 105.153 | 1.00 | 230.03 | O |
| HETATM | 10241 | O7 | NAG | A | 756 | −56.499 | −41.761 | 106.542 | 1.00 | 231.39 | O |
| HETATM | 10242 | C1 | NAG | B | 2333 | 2.726 | −34.155 | 80.541 | 1.00 | 210.90 | C |
| HETATM | 10243 | C2 | NAG | B | 2333 | 3.257 | −32.784 | 80.958 | 1.00 | 213.08 | C |
| HETATM | 10244 | C3 | NAG | B | 2333 | 3.122 | −32.649 | 82.482 | 1.00 | 213.30 | C |
| HETATM | 10245 | C4 | NAG | B | 2333 | 1.774 | −33.154 | 83.046 | 1.00 | 213.28 | C |
| HETATM | 10246 | C5 | NAG | B | 2333 | 0.810 | −33.910 | 82.081 | 1.00 | 213.01 | C |
| HETATM | 10247 | C6 | NAG | B | 2333 | −0.509 | −33.116 | 81.857 | 1.00 | 213.18 | C |
| HETATM | 10248 | C7 | NAG | B | 2333 | 5.189 | −31.487 | 80.006 | 1.00 | 214.04 | C |
| HETATM | 10249 | C8 | NAG | B | 2333 | 4.389 | −30.615 | 79.063 | 1.00 | 213.82 | C |
| HETATM | 10250 | N2 | NAG | B | 2333 | 4.634 | −32.620 | 80.483 | 1.00 | 213.60 | N |
| HETATM | 10251 | O3 | NAG | B | 2333 | 3.282 | −31.292 | 82.858 | 1.00 | 213.71 | O |
| HETATM | 10252 | O4 | NAG | B | 2333 | 2.025 | −33.955 | 84.198 | 1.00 | 212.99 | O |
| HETATM | 10253 | O5 | NAG | B | 2333 | 1.324 | −34.237 | 80.773 | 1.00 | 212.04 | O |
| HETATM | 10254 | O6 | NAG | B | 2333 | −1.061 | −32.660 | 83.111 | 1.00 | 213.25 | O |
| HETATM | 10255 | O7 | NAG | B | 2333 | 6.341 | −31.152 | 80.308 | 1.00 | 214.50 | O |
| HETATM | 10256 | C1 | NAG | B | 2334 | −19.386 | −37.495 | 50.517 | 1.00 | 191.44 | C |
| HETATM | 10257 | C2 | NAG | B | 2334 | −19.663 | −38.201 | 49.192 | 1.00 | 193.94 | C |
| HETATM | 10258 | C3 | NAG | B | 2334 | −21.135 | −38.064 | 48.799 | 1.00 | 194.11 | C |
| HETATM | 10259 | C4 | NAG | B | 2334 | −22.064 | −38.401 | 49.975 | 1.00 | 194.11 | C |
| HETATM | 10260 | C5 | NAG | B | 2334 | −21.661 | −37.680 | 51.272 | 1.00 | 193.74 | C |
| HETATM | 10261 | C6 | NAG | B | 2334 | −22.422 | −38.184 | 52.505 | 1.00 | 194.37 | C |
| HETATM | 10262 | C7 | NAG | B | 2334 | −18.246 | −38.258 | 47.103 | 1.00 | 196.59 | C |
| HETATM | 10263 | C8 | NAG | B | 2334 | −17.331 | −39.472 | 47.326 | 1.00 | 196.47 | C |
| HETATM | 10264 | N2 | NAG | B | 2334 | −18.762 | −37.623 | 48.201 | 1.00 | 195.42 | N |
| HETATM | 10265 | O3 | NAG | B | 2334 | −21.401 | −38.962 | 47.734 | 1.00 | 194.69 | O |
| HETATM | 10266 | O4 | NAG | B | 2334 | −23.417 | −38.138 | 49.636 | 1.00 | 194.38 | O |
| HETATM | 10267 | O5 | NAG | B | 2334 | −20.283 | −37.871 | 51.543 | 1.00 | 192.21 | O |
| HETATM | 10268 | O6 | NAG | B | 2334 | −23.685 | −38.748 | 52.175 | 1.00 | 195.26 | O |
| HETATM | 10269 | O7 | NAG | B | 2334 | −18.487 | −37.845 | 45.932 | 1.00 | 197.38 | O |
| HETATM | 10270 | C1 | NAG | B | 2335 | −24.483 | −40.157 | 47.480 | 1.00 | 257.33 | C |
| HETATM | 10271 | C2 | NAG | B | 2335 | −23.770 | −40.981 | 46.393 | 1.00 | 257.64 | C |
| HETATM | 10272 | C3 | NAG | B | 2335 | −24.236 | −42.461 | 46.251 | 1.00 | 257.77 | C |
| HETATM | 10273 | C4 | NAG | B | 2335 | −24.954 | −43.045 | 47.486 | 1.00 | 257.77 | C |
| HETATM | 10274 | C5 | NAG | B | 2335 | −25.732 | −41.943 | 48.218 | 1.00 | 257.46 | C |
| HETATM | 10275 | C6 | NAG | B | 2335 | −26.483 | −42.495 | 49.418 | 1.00 | 257.44 | C |
| HETATM | 10276 | C7 | NAG | B | 2335 | −24.877 | −40.349 | 44.189 | 1.00 | 257.76 | C |
| HETATM | 10277 | C8 | NAG | B | 2335 | −24.502 | −40.754 | 42.783 | 1.00 | 257.62 | C |
| HETATM | 10278 | N2 | NAG | B | 2335 | −23.865 | −40.251 | 45.105 | 1.00 | 257.86 | N |
| HETATM | 10279 | O3 | NAG | B | 2335 | −23.095 | −43.269 | 45.984 | 1.00 | 257.68 | O |
| HETATM | 10280 | O4 | NAG | B | 2335 | −25.791 | −44.202 | 47.263 | 1.00 | 258.43 | O |
| HETATM | 10281 | O5 | NAG | B | 2335 | −24.824 | −40.935 | 48.608 | 1.00 | 257.05 | O |
| HETATM | 10282 | O6 | NAG | B | 2335 | −27.755 | −42.928 | 48.979 | 1.00 | 257.18 | O |
| HETATM | 10283 | O7 | NAG | B | 2335 | −26.067 | −40.116 | 44.431 | 1.00 | 257.55 | O |
| HETATM | 10284 | C1 | MAN | B | 2336 | −26.450 | −44.361 | 45.976 | 1.00 | 259.29 | C |
| HETATM | 10285 | C2 | MAN | B | 2336 | −25.922 | −45.617 | 45.266 | 1.00 | 259.88 | C |
| HETATM | 10286 | C3 | MAN | B | 2336 | −26.495 | −45.661 | 43.845 | 1.00 | 260.42 | C |
| HETATM | 10287 | C4 | MAN | B | 2336 | −28.039 | −45.611 | 43.867 | 1.00 | 260.08 | C |
| HETATM | 10288 | C5 | MAN | B | 2336 | −28.640 | −44.638 | 44.911 | 1.00 | 259.37 | C |
| HETATM | 10289 | C6 | MAN | B | 2336 | −30.013 | −45.167 | 45.332 | 1.00 | 258.41 | C |
| HETATM | 10290 | O2 | MAN | B | 2336 | −26.247 | −46.798 | 45.980 | 1.00 | 259.57 | O |
| HETATM | 10291 | O3 | MAN | B | 2336 | −25.966 | −46.760 | 43.092 | 1.00 | 261.16 | O |
| HETATM | 10292 | O4 | MAN | B | 2336 | −28.544 | −45.279 | 42.583 | 1.00 | 260.25 | O |
| HETATM | 10293 | O5 | MAN | B | 2336 | −27.865 | −44.450 | 46.100 | 1.00 | 259.24 | O |
| HETATM | 10294 | O6 | MAN | B | 2336 | −30.926 | −44.124 | 45.613 | 1.00 | 257.34 | O |
| HETATM | 10295 | C1 | MAN | B | 2337 | −25.059 | −46.294 | 42.038 | 1.00 | 261.47 | C |
| HETATM | 10296 | C2 | MAN | B | 2337 | −25.795 | −46.163 | 40.692 | 1.00 | 261.59 | C |
| HETATM | 10297 | C3 | MAN | B | 2337 | −25.949 | −47.551 | 40.045 | 1.00 | 261.51 | C |
| HETATM | 10298 | C4 | MAN | B | 2337 | −24.625 | −48.341 | 39.958 | 1.00 | 261.09 | C |
| HETATM | 10299 | C5 | MAN | B | 2337 | −23.737 | −48.251 | 41.216 | 1.00 | 260.60 | C |
| HETATM | 10300 | C6 | MAN | B | 2337 | −22.271 | −48.517 | 40.839 | 1.00 | 259.52 | C |
| HETATM | 10301 | O2 | MAN | B | 2337 | −25.147 | −45.241 | 39.827 | 1.00 | 261.32 | O |
| HETATM | 10302 | O3 | MAN | B | 2337 | −26.534 | −47.408 | 38.764 | 1.00 | 261.88 | O |
| HETATM | 10303 | O4 | MAN | B | 2337 | −24.921 | −49.706 | 39.705 | 1.00 | 260.85 | O |
| HETATM | 10304 | O5 | MAN | B | 2337 | −23.809 | −46.999 | 41.909 | 1.00 | 261.19 | O |
| HETATM | 10305 | O6 | MAN | B | 2337 | −21.892 | −49.848 | 41.194 | 1.00 | 258.27 | O |
| HETATM | 10306 | C1 | MAN | B | 2338 | −31.613 | −44.399 | 46.860 | 1.00 | 256.65 | C |
| HETATM | 10307 | C2 | MAN | B | 2338 | −32.496 | −43.209 | 47.272 | 1.00 | 256.41 | C |
| HETATM | 10308 | C3 | MAN | B | 2338 | −33.655 | −43.574 | 48.228 | 1.00 | 256.15 | C |
| HETATM | 10309 | C4 | MAN | B | 2338 | −33.645 | −44.986 | 48.852 | 1.00 | 255.98 | C |
| HETATM | 10310 | C5 | MAN | B | 2338 | −32.722 | −46.010 | 48.174 | 1.00 | 255.84 | C |
| HETATM | 10311 | C6 | MAN | B | 2338 | −33.380 | −47.387 | 48.117 | 1.00 | 255.28 | C |
| HETATM | 10312 | O2 | MAN | B | 2338 | −32.990 | −42.538 | 46.123 | 1.00 | 256.25 | O |
| HETATM | 10313 | O3 | MAN | B | 2338 | −34.898 | −43.379 | 47.580 | 1.00 | 256.10 | O |
| HETATM | 10314 | O4 | MAN | B | 2338 | −33.316 | −44.899 | 50.226 | 1.00 | 255.61 | O |
| HETATM | 10315 | O5 | MAN | B | 2338 | −32.372 | −45.596 | 46.865 | 1.00 | 256.21 | O |
| HETATM | 10316 | O6 | MAN | B | 2338 | −32.847 | −48.217 | 49.125 | 1.00 | 254.72 | O |
| HETATM | 10317 | CU | CU | B | 1 | −34.550 | −36.792 | 79.967 | 1.00 | 174.41 | CU |
| HETATM | 10318 | CU | CU | A | 757 | −45.781 | −46.777 | 90.870 | 1.00 | 171.28 | CU |
| HETATM | 10319 | CA | CA | A | 758 | −44.908 | −24.759 | 63.651 | 1.00 | 172.22 | CA |

| CONECT | 647 | 10319 | | | | | | | | | | |
|---|---|---|---|---|---|---|---|---|---|---|---|---|
| CONECT | 764 | 10319 | | | | | | | | | | |
| CONECT | 794 | 10319 | | | | | | | | | | |
| CONECT | 802 | 10319 | | | | | | | | | | |
| CONECT | 803 | 10319 | | | | | | | | | | |
| CONECT | 1010 | 1206 | | | | | | | | | | |
| CONECT | 1206 | 1010 | | | | | | | | | | |
| CONECT | 1589 | 10214 | | | | | | | | | | |
| CONECT | 1651 | 2302 | | | | | | | | | | |
| CONECT | 1805 | 10318 | | | | | | | | | | |
| CONECT | 2302 | 1651 | | | | | | | | | | |
| CONECT | 3589 | 3793 | | | | | | | | | | |
| CONECT | 3793 | 3589 | | | | | | | | | | |
| CONECT | 4415 | 5069 | | | | | | | | | | |
| CONECT | 5069 | 4415 | | | | | | | | | | |
| CONECT | 5992 | 10242 | | | | | | | | | | |
| CONECT | 6184 | 6386 | | | | | | | | | | |
| CONECT | 6386 | 6184 | | | | | | | | | | |
| CONECT | 6730 | 6759 | | | | | | | | | | |
| CONECT | 6759 | 6730 | | | | | | | | | | |
| CONECT | 7181 | 10317 | | | | | | | | | | |
| CONECT | 7599 | 10317 | | | | | | | | | | |
| CONECT | 7725 | 8909 | | | | | | | | | | |
| CONECT | 8504 | 10256 | | | | | | | | | | |
| CONECT | 8909 | 7725 | | | | | | | | | | |
| CONECT | 8945 | 10161 | | | | | | | | | | |
| CONECT | 10161 | 8945 | | | | | | | | | | |
| CONECT | 10214 | 15891021510225 | | | | | | | | | | |
| CONECT | 10215 | 102141021610222 | | | | | | | | | | |
| CONECT | 10216 | 102151021710223 | | | | | | | | | | |
| CONECT | 10217 | 102161021810224 | | | | | | | | | | |
| CONECT | 10218 | 102171021910225 | | | | | | | | | | |
| CONECT | 10219 | 1021810226 | | | | | | | | | | |
| CONECT | 10220 | 102211022210227 | | | | | | | | | | |
| CONECT | 10221 | 10220 | | | | | | | | | | |
| CONECT | 10222 | 1021510220 | | | | | | | | | | |
| CONECT | 10223 | 10216 | | | | | | | | | | |
| CONECT | 10224 | 1021710228 | | | | | | | | | | |
| CONECT | 10225 | 1021410218 | | | | | | | | | | |
| CONECT | 10226 | 10219 | | | | | | | | | | |
| CONECT | 10227 | 10220 | | | | | | | | | | |
| CONECT | 10228 | 102241022910239 | | | | | | | | | | |
| CONECT | 10229 | 102281023010236 | | | | | | | | | | |
| CONECT | 10230 | 102291023110237 | | | | | | | | | | |
| CONECT | 10231 | 102301023210238 | | | | | | | | | | |
| CONECT | 10232 | 102311023310239 | | | | | | | | | | |
| CONECT | 10233 | 1023210240 | | | | | | | | | | |
| CONECT | 10234 | 102351023610241 | | | | | | | | | | |
| CONECT | 10235 | 10234 | | | | | | | | | | |
| CONECT | 10236 | 1022910234 | | | | | | | | | | |
| CONECT | 10237 | 10230 | | | | | | | | | | |
| CONECT | 10238 | 10231 | | | | | | | | | | |
| CONECT | 10239 | 1022810232 | | | | | | | | | | |
| CONECT | 10240 | 10233 | | | | | | | | | | |
| CONECT | 10241 | 10234 | | | | | | | | | | |
| CONECT | 10242 | 59921024310253 | | | | | | | | | | |
| CONECT | 10243 | 102421024410250 | | | | | | | | | | |
| CONECT | 10244 | 102431024510251 | | | | | | | | | | |
| CONECT | 10245 | 102441024610252 | | | | | | | | | | |
| CONECT | 10246 | 102451024710253 | | | | | | | | | | |
| CONECT | 10247 | 1024610254 | | | | | | | | | | |
| CONECT | 10248 | 102491025010255 | | | | | | | | | | |
| CONECT | 10249 | 10248 | | | | | | | | | | |
| CONECT | 10250 | 1024310248 | | | | | | | | | | |
| CONECT | 10251 | 10244 | | | | | | | | | | |
| CONECT | 10252 | 10245 | | | | | | | | | | |
| CONECT | 10253 | 1024210246 | | | | | | | | | | |
| CONECT | 10254 | 10247 | | | | | | | | | | |
| CONECT | 10255 | 10248 | | | | | | | | | | |
| CONECT | 10256 | 85041025710267 | | | | | | | | | | |
| CONECT | 10257 | 102561025810264 | | | | | | | | | | |
| CONECT | 10258 | 102571025910265 | | | | | | | | | | |
| CONECT | 10259 | 102581026010266 | | | | | | | | | | |
| CONECT | 10260 | 102591026110267 | | | | | | | | | | |
| CONECT | 10261 | 1026010268 | | | | | | | | | | |
| CONECT | 10262 | 102631026410269 | | | | | | | | | | |
| CONECT | 10263 | 10262 | | | | | | | | | | |
| CONECT | 10264 | 1025710262 | | | | | | | | | | |
| CONECT | 10265 | 10258 | | | | | | | | | | |
| CONECT | 10266 | 10259 | | | | | | | | | | |
| CONECT | 10267 | 1025610260 | | | | | | | | | | |
| CONECT | 10268 | 10261 | | | | | | | | | | |
| CONECT | 10269 | 10262 | | | | | | | | | | |
| CONECT | 10270 | 1027110281 | | | | | | | | | | |
| CONECT | 10271 | 102701027210278 | | | | | | | | | | |
| CONECT | 10272 | 102711027310279 | | | | | | | | | | |
| CONECT | 10273 | 102721027410280 | | | | | | | | | | |
| CONECT | 10274 | 102731027510281 | | | | | | | | | | |
| CONECT | 10275 | 1027410282 | | | | | | | | | | |
| CONECT | 10276 | 102771027810283 | | | | | | | | | | |
| CONECT | 10277 | 10276 | | | | | | | | | | |
| CONECT | 10278 | 1027110276 | | | | | | | | | | |
| CONECT | 10279 | 10272 | | | | | | | | | | |
| CONECT | 10280 | 1027310284 | | | | | | | | | | |
| CONECT | 10281 | 1027010274 | | | | | | | | | | |
| CONECT | 10282 | 10275 | | | | | | | | | | |
| CONECT | 10283 | 10276 | | | | | | | | | | |
| CONECT | 10284 | 102801028510293 | | | | | | | | | | |
| CONECT | 10285 | 102841028610290 | | | | | | | | | | |
| CONECT | 10286 | 102851028710291 | | | | | | | | | | |
| CONECT | 10287 | 102861028810292 | | | | | | | | | | |
| CONECT | 10288 | 102871028910293 | | | | | | | | | | |
| CONECT | 10289 | 1028810294 | | | | | | | | | | |
| CONECT | 10290 | 10285 | | | | | | | | | | |
| CONECT | 10291 | 1028610295 | | | | | | | | | | |
| CONECT | 10292 | 10287 | | | | | | | | | | |
| CONECT | 10293 | 1028410288 | | | | | | | | | | |
| CONECT | 10294 | 1028910306 | | | | | | | | | | |
| CONECT | 10295 | 102911029610304 | | | | | | | | | | |
| CONECT | 10296 | 102951029710301 | | | | | | | | | | |
| CONECT | 10297 | 102961029810302 | | | | | | | | | | |
| CONECT | 10298 | 102971029910303 | | | | | | | | | | |
| CONECT | 10299 | 102981030010304 | | | | | | | | | | |
| CONECT | 10300 | 1029910305 | | | | | | | | | | |
| CONECT | 10301 | 10296 | | | | | | | | | | |
| CONECT | 10302 | 10297 | | | | | | | | | | |
| CONECT | 10303 | 10298 | | | | | | | | | | |
| CONECT | 10304 | 1029510299 | | | | | | | | | | |
| CONECT | 10305 | 10300 | | | | | | | | | | |
| CONECT | 10306 | 102941030710315 | | | | | | | | | | |
| CONECT | 10307 | 103061030810312 | | | | | | | | | | |
| CONECT | 10308 | 103071030910313 | | | | | | | | | | |
| CONECT | 10309 | 103081031010314 | | | | | | | | | | |
| CONECT | 10310 | 103091031110315 | | | | | | | | | | |
| CONECT | 10311 | 1031010316 | | | | | | | | | | |
| CONECT | 10312 | 10307 | | | | | | | | | | |
| CONECT | 10313 | 10308 | | | | | | | | | | |
| CONECT | 10314 | 10309 | | | | | | | | | | |
| CONECT | 10315 | 1030610310 | | | | | | | | | | |
| CONECT | 10316 | 10311 | | | | | | | | | | |
| CONECT | 10317 | 7181 | 7599 | | | | | | | | | |
| CONECT | 10318 | 1805 | | | | | | | | | | |
| CONECT | 10319 | 647 | 764 | 794 | 802 | | | | | | | |
| CONECT | 10319 | 803 | | | | | | | | | | |
| MASTER | | 926 | 0 | 11 | 15 | 72 | 0 | 11 | 610317 | 2 | 134 | 111 |
| END | | | | | | | | | | | | |

### INCORPORATION OF REFERENCES

All publications and patent documents cited in this application are incorporated by reference in their entirety to the same extent as if the contents of each individual publication or patent document were incorporated herein.

We claim:

1. A composition comprising a human Factor VIII, in a crystalline form, wherein the human Factor VIII lacks at least a portion of B-domain.

2-7. (canceled)

8. A computer-readable medium comprising computer-readable data defined by structural coordinates of atoms of human Factor VIII according to Table 2, +/−a root mean square deviation for alpha carbon atoms of less than 2 Angstroms, or selected coordinates thereof.

9-13. (canceled)

14. A method for constructing a three-dimensional structural representation of a Factor VIIIa-Factor IXa complex, the method comprising the steps of:
(a) providing a three-dimensional structural representation of human Factor VIII, or a region thereof,
(b) providing a three-dimensional structural representation of Factor IXa, or a region thereof, and
(c) fitting the three-dimensional structural representation from step (a) to the three-dimensional structural representation from step (b).

15-19. (canceled)

20. A method for modifying human Factor VIII to alter its interaction with Factor IXa, the method comprising the steps of:
(a) providing a structural representation of human Factor VIII, or a region thereof,
(b) fitting the structural representation of step (a) to a three-dimensional structural representation of Factor IXa, or a region thereof, and
(d) computationally modifying the structural representation of step (a) to increase or decrease its interaction with the three-dimensional structural representation of Factor IXa, or a region thereof.

21. The method of claim 20, wherein the structural representation of step (a) is defined by structural coordinates of atoms of human Factor VIII according to Table 2, +/−a root mean square deviation for alpha carbon atoms of less than 2 Angstroms, or selected coordinates thereof.

22. The method of claim 20, wherein the structural representation of step (a) is defined by structural coordinates of one or more atoms selected from the group consisting of atoms of residues 558-565, 707-712 and 1811-1819 according to Table 2.

23. The method of claim 20, wherein the three-dimensional structural representation of Factor IXa, or a region thereof, is defined at least by the Gla domain of Factor IXa.

24. The method of claim 20, wherein the three-dimensional structural representation of Factor IXa, or a region thereof, is defined at least by the light chain of Factor IXa.

25. The method of claim 20, wherein the structural representation of step (a) is modified to increase its interaction with the three-dimensional structural representation of Factor IXa, or a region thereof.

26. A modified human Factor VIII with increased interaction with Factor IXa according to the method of claim 25.

27. A method for evaluating the activity of a modified human Factor VIII, the method comprising the steps of:
(a) providing a structural representation of human Factor VIII, or a region thereof,
(b) computationally modifying the structural representation of step (a) to introduce one or more amino acid modifications; and
(c) evaluating the activity of the modified human Factor VIII based on the modified structural representation from step (b).

28. The method of claim 27, wherein the structural representation of step (a) is defined by coordinates of atoms of human Factor VIII according to Table 2, +/−a root mean square deviation for alpha carbon atoms of less than 2 Angstroms, or selected coordinates thereof.

29. The method of claim 27, wherein the evaluation of the activity of the modified human Factor VIII is based on an evaluation of its interaction with Factor IXa.

30. The method of claim 27, wherein the evaluation of the activity of the modified human Factor VIII is based on an evaluation of its interaction with a phospholipid membrane.

31. The method of claim 27, wherein the evaluation of the activity of the modified human Factor VIII is based on an evaluation of its interaction with von Willebrand Factor (vWF).

32. The method of claim 27, wherein the method further comprises a step of identifying a modified Factor VIII with an improved property.

33. (canceled)

34. A method of predicting a three dimensional structure of a human Factor VIII homologue or analogue of unknown structure, the method comprising the steps of:
(a) aligning an amino acid sequence of a target human Factor VIII homologue or analogue of unknown structure with the amino acid sequence of human Factor VIII defined by coordinates according to Table 2, +/−a root mean square deviation for alpha carbon atoms of less than 2 Angstroms, or selected coordinates thereof, to match one or more homologous regions;
(b) modeling the structure of the matched one or more homologous regions of the target human Factor VIII homologue or analogue of unknown structure on the corresponding regions of the human Factor VIII as defined by coordinates according to Table 2, +/−a root mean square deviation for alpha carbon atoms of less than 2 Angstroms, or selected coordinates thereof, and
(c) determining a structural conformation for said target human Factor VIII homologue or analogue of unknown structure which substantially preserves the structure of said matched one or more homologous regions.

35. (canceled)

36. A method for designing a mimetic compound of human Factor VIII, the method comprising the steps of:
(a) providing a selected human Factor VIII structure that is associated with a biological activity of human Factor VIII;
(b) superimposing a three-dimensional structure of a compound on the selected human Factor VIII structure;
(c) modifying the three-dimensional structure of the compound such that the modified three-dimensional structure comprises a structural confirmation substantially mimicking the selected human Factor VIII structure.

37-38. (canceled)

39. A mimetic antibody of human Factor VIII designed by the method of claim 37.

40. A method for rational drug design, the method comprising the steps of:
(a) providing selected coordinates of a human Factor VIII structure;
(b) providing a plurality of moieties;
(c) fitting the structure of each of the plurality of moieties to the selected coordinates;
(d) selecting one or more moieties that fit into the selected coordinates; and
(e) assembling the one or more moieties selected from step (d) into a single molecule to form a candidate modulator molecule.

41-43. (canceled)

44. A method for producing a computer readable database comprising a structural representation of at least one compound capable of binding human Factor VIII, the method comprising the steps of:
(a) introducing into a computer program selected coordinates of a human Factor VIII structure;
(b) fitting a three-dimensional model of at least one binding test compound into the selected coordinates;
(d) assessing whether said test compound model fits spatially into the selected coordinates; and
(e) storing a structural representation of a compound that fits into the selected coordinates.

45. A computer readable database produced by the method of claim 44.


SEQUENCE LISTING (3 sequences)

SEQ ID NO: 1 - protein, 740 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPR

SEQ ID NO: 2 - protein, 684 aa, Homo sapiens
EITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFLTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 14 aa, Artificial Sequence
  Linker Sequence
SFSQNPPVLKRHQR